(19) United States
(12) Patent Application Publication
(10) Pub. No.: US 2006/0177446 A1
(43) Pub. Date: Aug. 10, 2006

(54) Polypeptides that bind an anti-tissue factor antibody and uses thereof

(75) Inventors: Charles Eigenbrot, Burlingame, CA (US); Yu-Ju G. Meng, Albany, CA (US); Daniel K. Kirchhofer, Los Altos, CA (US); Leonard G. Presta, San Francisco, CA (US)

Correspondence Address: MERCHANT & GOULD PC, P.O. BOX 2903, MINNEAPOLIS, MN, 55402-0903, US

(73) Assignee: GENENTECH, INC., SOUTH SAN FRANCISCO, CA (US)

(21) Appl. No.: 11/290,770

(22) Filed: Nov. 29, 2005

Related U.S. Application Data
(63) Continuation of application No. PCT/US04/16783, filed on May 26, 2004.
(60) Provisional application No. 60/474,534, filed on May 30, 2003.
(60) Provisional application No. 60/476,025, filed on Jun. 4, 2003.

(51) Int. Cl. A61K 39/395; C07K 16/22

(52) U.S. Cl. 424/145.1; 530/388.25

(57) ABSTRACT

The present invention relates to antibodies and polypeptides useful in blocking the binding of antibodies to anti-tissue factor, decreasing the activity of the anti-tissue factor antibodies and/or neutralizing the activity of an anti-tissue factor antibody. The present invention also relates to nucleic acids encoding the antibodies and polypeptides and compositions comprising the antibodies or polypeptides or nucleic acid molecules. The present invention provides methods of treating a hypercoagulable state or an inflammatory disease using the antibodies and polypeptides of this invention and other methods of using said antibodies and polypeptides.

This application claims benefit from U.S. Provisional Application No. 60/474,534, filed May 30, 2003 and 60/476,025, filed Jun. 4, 2003.

### FIELD OF THE INVENTION

The present invention relates to polypeptides and antibodies that bind an-anti-tissue factor antibody, compositions comprising the same and methods for using the polypeptides and the compositions.

### BACKGROUND AND INTRODUCTION OF THE INVENTION

Tissue factor (TF), the cell surface-expressed cofactor for the serine protease factor VIIa (FVIIa), triggers blood coagulation by combining with F.VIIa to activate the substrates factors IX, X and F.VII. The ensuing coagulation reactions result in the formation of a polymerized fibrin meshwork and platelet aggregates, which together form a hemostatic plug. The activity of TF/FVIIa has been implicated in numerous diseases. For instance, TF/F.VIIa plays an important role in cardiovascular diseases in which blood clot formation may become life-threatening. Atherosclerotic plaques contain TF (e.g., Wilcox, J. N. et al. Proc. Natl. Acad. Sci. U.S.A. 86, 2839-2843 (1989)), which upon plaque rupture may trigger the formation of an occlusive thrombus leading to myocardial infarction (e.g., Badimon, J. J. Circulation 99, 1780-1787 (1999); Ardissino, D. Blood 98, 2726-2729 (2001)). The cellular sources of TF in plaques appears to be macrophages and smooth muscle cells (e.g., Kaikita, K. et al., Arterioscler. Thromb. Vasc. Biol. 17, 2232-2237 (1997); Moreno, P. R. et al., Circulation 94, 2090-3097 (1996); Toschi, V. et al. Circulation 95, 594-599 (1997)), but TF is also found in the acellular, highly thrombogenic lipid-rich core (e.g., Moreno, P. R. et al., Circulation 94, 2090-3097 (1996); Toschi, V. et al. Circulation 95, 594-599 (1997); Marmur J. D. et al., Circulation 94, 1226-1232 (1996)). For patients with unstable angina the predisposition to thrombotic episodes may not only derive from TF in atherosclerotic plaques (e.g., Moreno, P. R. et al., Circulation 94, 2090-3097 (1996); Annex, B. H. et al. Circulation 91, 619-622 (1995)) but also from elevated levels of circulating TF (e.g., Leatham, E. W. et al. Br. Heart J. 73, 10-13 (1995); Santucci, R. A. et al., Thromb. Haemost. 83, 445454 (2000); Falciani, M. et al., Thromb. Haemost 79, 495-499 (1998); Jude, B. et al. Circulation 90, 1662-1668 (1994); Soejima, H. et al. Circulation 99, 2908-2913 (1999)). It was recently demonstrated that circulating TF can incorporate into growing platelet thrombi and may thus sustain further thrombus growth. Furthermore, it has been suggested that TF plays a role in cancer metastasis, tumor angiogenesis and in inflammatory diseases, such as sepsis, rheumatoid arthritis (e.g., Zacharski, L. R. et al. Clin. Immunol. Immunopathol. 63, 155-162 (1992)) and sickle cell anemia (e.g., Key, N. S. et al. Blood 91, 4216-4223 (1998)). In some of these diseases, the effects of the TF/FVIIa complex may include the activation of intracellular signaling pathways mediated by protease activated receptors (PAR), a family of G-protein coupled receptors. It has been demonstrated that certain members of the PAR family can be activated by the TF/FVIIa complex itself as well as by coagulation enzymes generated by TF/FVIIa activity, such as F.Xa and thrombin (e.g., Riewald, M. et al. Trends Cardiovasc Med 12, 149-154 (2002); Petersen, L. C. et al. Trends Cardiovasc Med 10, 47-52 (2000); Wiger, M. T. et al. Trends Cardiovasc Med 10, 360-365 (2000)). Therefore, interference with TF/V.VIIa function can be beneficial for treatment of a wide range of diseases.

A fully humanized anti-TF antibody D3H44 has been produced (Presta, L. et al. Thromb. Haemost. 85, 379-79 (2001)). In agreement with the excellent potency of the murine D3 antibody from which it was derived (Kirchhofer, D. et al. Thromb. Haemost. 84, 1072-1081 (2000)), D3H44 is a potent anticoagulant both in vitro (Presta, L. et al., (2001) supra) and in vivo (Bullens, S. et al. Thromb. Haemost. (Suppl) Abstract #P1388 (2001)). The recent elucidation of the 1.85 Å resolution crystal structure of the complex of D3H44-Fab with TF provided detailed insights into the antigen combining site and the TF residues engaged in antibody binding (Faelber, K. et al. J. Mol. Biol. 313, 83-97 (2001)). The TF epitope, located in the C-terminal fibronection type HIII domain, lies outside the TF-FVIIa interface and largely overlaps with the substrate interaction region of TF centered at Lys165 and Lys166 (Kirchhofer, D. et al. Biochemistry 39, 7380-7387 (2000); Roy S. et al. J. Biol. Chem. 266, 22063-22066 (1991); Ruf W. et al. J. Biol. Chem. 267, 6375-6381 (1992)). The substrate recognition region of TF region is also important for the interaction of F.Xa with the TF/FVII complex and D3H44 was shown to completely inhibit F.VII conversion by F.Xa (Kirchhofer D. et al. Biochemistry 40, 675-682 (2001)). This TF region presumably interacts with the gamma-carboxyglutamic acid-rich (Gla) (Ruf W. et al. Biochemistry 38, 1957-1966 (1999); Huang Q. et al. J. Biol. Chem. 271, 21752-21757 (1996); Martin D. M. A. et al. Biochemistry 32, 13949-13955 (1993)) and EGF-I domains (Zhong D. et al. J. Biol. Chem. 277, 3622-3631 (2002)) of substrates and activators. Such an arrangement seems key to providing optimal substrate/active-site interactions taking place -70-80Å above the membrane surface (Banner D. W. et al. Nature 380, 4146 (1996); McCallum C. et al. J. Biol. Chem. 271, 28168-28175 (1996)).

In vivo studies with specific inhibitors of TF suggested that an anti-TF antibody like D3H44 might be safer than the currently used anticoagulants (Harker L. A. et al. Haemostasis 26 (suppl. 1), 76-82, (1996); Himber J. et al. Thromb. Haemost. 78, 1142-1149 (1997)). Nevertheless, it was observed that high doses of D3H44 in chimpanzees can interfere with normal hemostasis (Bullens S. et al. (2001), supra). It is an object of this invention to develop a better and/or alternative agent for facilitating the restoration of normal hemostasis after disruption of hemostasis by an antibody against tissue factor. It is an object of this invention that the agent is not a procoagulant. It is also an object of this invention to develop compositions and methods for inhibiting the binding of an anti-tissue factor antibody to tissue factor. It is also an object of this invention to develop alternative and/or better methods of controlling the anticoagulant activity of an anti-tissue factor antibody. It is an object of this invention to provide alternative and/or better methods for treating a hypercoagulable condition in a mammal. It is an object of this invention to provide alternative and/or better methods for treating an inflammatory disease in a mammal. It is a further object of the present invention to provide an anti-idiotypic antibody, which recognizes complementarity-determining regions (CDR's) of an antibody against a tissue factor, and, preferably, competitively and specifically inhibits the binding of the anti-tissue factor antibody to a tissue factor.

### SUMMARY OF THE INVENTION

The present invention provides polypeptides and antibodies that can neutralize the anti-coagulant activity of an anti-tissue factor antibody. The present invention provides polypeptides and antibodies that can specifically bind to the variable region of an antibody that binds to an extracellular domain sequence of a tissue factor. In a further embodiment, the anti-tissue factor antibody binds to a region of tissue factor that binds Factor X. In one embodiment, the antibodies of this invention inhibit the anticoagulant activity of an anti-tissue factor antibody. According to one embodiment, the anti-tissue factor antibody comprises the variable region of D3H44. According to another embodiment, the antibody of this invention binds to one or more specified regions within an antibody comprising the variable region of D3H44. According to one embodiment, the antibody of this invention binds to the anti-tissue factor antibody with an affinity of 1 μM or less, 500 nM or less, 100 nM or less, 50 nM or less, 10 nM or less, 5 nM or less, 1 nM or less. According to a further embodiment, the antibody of this invention inhibits binding of an anti-tissue factor antibody to tissue factor. The present invention also provides specific amino acid sequences of antibodies and polypeptides useful according to this invention. According to another aspect, the antibodies and polypeptides are in crystalline form.

According to one embodiment, an antibody of this invention is a monoclonal antibody. According to another aspect, the antibody of this invention can additionally be any one or a combination of a humanized antibody and an antibody fragment. In another embodiment, the antibody of this invention is a bispecific antibody. In one embodiment, an antibody of this invention has a framework region derived from one or more human antibodies.

Antibodies of this invention include antibodies that can bind essentially the same epitope and/or have the biological characteristics as the 6A6 antibody produced by a deposited hybridoma cell line designated 6A6.3E11.2E12.

According to one aspect, antibodies or polypeptides of this invention can be conjugated to an agent selected from the group consisting of a growth inhibitory agent, a cytotoxic agent, a detection agent, an agent that improves the bioavailability of the antibody or polypeptide and an agent that improves the half-life of the antibody or polypeptide to facilitate the treatment or diagnosis of diseases or illnesses. According a further embodiment, the cytotoxic agent can be selected from the group consisting of a toxin, an antibiotic and a radioactive isotope.

The present invention provides nucleic acid molecules encoding the antibodies or the polypeptides of this invention. The present invention also provides vectors comprising the nucleic acid molecule of this invention, wherein the vectors optionally contain sequences to cause the expression of the polypeptides and antibodies of this invention from the vector. The present invention further provides host cells comprising the vectors of this invention.

The present invention includes compositions comprising the polypeptides, antibodies or nucleic acid molecules of this invention. The compositions of this invention can additionally include a second therapeutic agent, such as a procoagulant. The compositions of this invention can optionally include a pharmaceutically acceptable carrier.

The present invention includes articles of manufacture comprising:

(a) a composition of matter selected from the group consisting of an antibody of this invention, a polypeptide of this invention, a nucleic acid molecule of this invention and an inactive tissue factor (iTF) of this invention;

(b) a container containing said composition; and

(c) a label affixed to said container, or a package insert included in said container referring to the use of the antibody, the polypeptide, or iTF in the treatment of a hypercoagulable state, an inflammatory disease or as a diagnostic agent, optionally including an anti-tissue factor antibody that binds to the antibody, polypeptide or iTF. The present invention also includes kits for performing macromolecular substrate assays, prothrombin assays or whole blood clotting assay using the antibodies or polypeptides or iTF of this invention, optionally including an anti-tissue factor antibody that binds to the antibody, polypeptide or iTF. An iTF according to this invention is a tissue factor that has been mutated so that it has substantially less procoagulant activity compared to a soluble tissue factor, but it can still bind to an anti-tissue factor antibody (ATF) and decrease the ATF anticoagulant activity. In one preferred embodiment, iTF is a soluble tissue factor that has been mutated at its Factor VIIa binding site so that Factor VIIa binding is decreased. In another preferred embodiment, the iTF binds to an anti-tissue factor antibody with an affinity that is similar to a soluble tissue factor.

The present invention provides a method for producing an antibody or a polypeptide according to this invention.

The present invention also provides methods for treating a hypercoagulable state in a mammal in need of treatment therefor comprising the step of administering a therapeutically effective amount of an anti-tissue factor antibody and subsequently a therapeutically effective amount of an antagonist agent selected from the group consisting of an antibody of this invention, a polypeptide of this invention, and a iTF of this invention. The present invention also provides methods for treating an inflammatory disease in a mammal in need of treatment therefor comprising the step of administering a therapeutically effective amount of an anti-tissue factor antibody and subsequently a therapeutically effective amount of an antagonist agent selected from the group consisting of an antibody of this invention, a polypeptide of this invention, and a iTF of this invention. According to one preferred embodiment, the mammal is suffering from an illness selected from the group consisting of atherosclerosis, sepsis, acute coronary syndrome, disseminated intravascular coagulation (DIC), sickle cell disease, venous thromboembolism, myocardial infarction, deep vein thrombosis (DVT), an inflammatory disease and cancer.

The present invention provides methods for inhibiting the anticoagulant activity of an ATF antibody in a mammal in need of treatment therefor comprising the step of administering a therapeutically effective amount of an agent selected from the group consisting of an antibody of this invention, a polypeptide of this invention, and a iTF of this invention. The present invention provides uses for antibodies and polypeptides of the invention during extracorporeal circulation of a patient. In one embodiment, one or more antibodies or polypeptides of the invention is administered to the mammal being treated in an amount sufficient to decrease the anticoagulant activity of an anti-tissue factor antibody prior to or during extracorporeal circulation as needed during, e.g., a cardiopulmonary bypass surgery, organ transplant surgery or other prolonged surgeries.

The present invention provides the use of the antibodies and polypeptides of this invention in diagnostic methods. In one embodiment, the antibodies and polypeptides of this invention are used in vitro assays to detect anti-tissue factor antibodies in a biological sample including a body fluid (e.g., plasma or serum) or tissue (e.g., a biopsy sample) of a patient being treated with anti-tissue factor antibodies. Antibodies and polpeptides of this invention can also be used for purifying anti-tissue factor antibodies.

### BRIEF DESCRIPTION OF THE DRAWINGS

FIG. 1 Binding of anti-TF antibodies to 6A6 coated on an ELISA plate. All antibodies analyzed were antibody Fabs except the Herceptin® antibody which is a full length human antibody specific for the HER2 receptor. D3Ch is a chimeric antibody consisting of the D3H44 constant domains and murine D3 variable domains. D3H13 and D3H18 are variants of the D3H44 antibody. The two chimeric antibodies huV_{L}muV_{H} and muV_{L}huV_{H} are composed of the same D3H44 constant domains, but they combine a human and a murine variable domain.

FIG. 2 Neutralization of D3H44 by 6A6 in TF-FVIIa-mediated activation of F.X. 10 nM D3H44 (Fab, F(ab′)₂ and IgG4b) was incubated with mTF(1-263), F.VIIa (0.02 nM) and increasing concentrations of 6A6 antibody for 25 min in HBSA buffer. F.X (200 nM) was added and at various time points reaction aliquots were withdrawn and analyzed for F.Xa in a second stage chromogenic substrate assay. The linear rates of initial F.Xa formation were determined and are expressed as fractional activities (vi/vo) open circles, D3H44-F(ab′)₂; triangles, D3H44-IgG4b; filled circles, D3H44-Fab; squares, 5G6 antibody.

FIG. 3 Neutralization of D3H44 anticoagulant activity by 6A6 antibody in whole blood and plasma. (a) Endotoxin-mediated whole blood clotting assay. Endotoxin exposure for 3 hours reduced clotting time by about 50%. Addition of 10 nM D3H44 (F(ab′)₂, Fab or IgG4b) prolonged clotting time to control values. 6A6 antibody (200 nM) alone neither prolonged nor reduced clotting times, but effectively neutralized D3H44. Unpaired 2-tailed t-test: *p<0.05; **p<0.01. (b) Prothrombin time (PT). Addition of 300 nM D3H44-F(ab′)₂ 300 nM D3H44-IgG4b, or 600 nM D3H44-Fab resulted in prolongation of the PT in citrated human plasma. 6A6 antibody at a concentration of 1.7 μM fully restored normal PT values. Dark bars indicate no 6A6, light bars indicate that 6A6 was present.

FIG. 4 Representative electron density from the 6A6-Fab/D3H44-Fab crystal structure. Shown is section of CDR-H1 from the final model with 2Fo-Fc density contoured at 1.0 rmsd.

FIG. 5 Relationship between 6A6-Fab and D3H44-Fab in the complex is head-to-head with a 90° twist. 6A6-Fab is the upper structure and D3H44-Fab is the lower structure, with the darker colors of each representing their respective heavy chains. See also, FIG. 5 of J. Mol. Bol. (2003) 331:433-446, 438 for color reproduction.

FIG. 6 Open book views of the solvent accessible surfaces buried in the (a) 6A6-Fab/D3H44-Fab and (b) TF/D3H44-Fab complexes. The contact zones are shaded darker. See also, FIG. 6 of J. Mol. Bol. (2003) 331:433-446, 439 for color reproduction wherein the contact zones are colored in blue for positive electrostatic potential and red for negative electrostatic potential.

FIG. 7 6A6 side chains used in binding D3H44. The globular structure is D3H44 is represented in (a) and (b), with side-chain atoms of 6A6-Fab drawn in (a) and side-chain atoms of tissue factor drawn in (b). See also, FIG. 7 of J. Mol. Bol. (2003) 331:433-446, 439 for color reproduction wherein a solvent accessible surface of D3H44-Fab is colored by CDR, with yellow shades in V_{H} (lightest shade CDR-H1 to darkest shade CDR-H3) and red shades in V_{L} (pinkest shade for CDR-L1 and most red shade for CDR-L3), and otherwise V_{H} is dark grey and V_{L} is light grey. Only side chain atoms of the binding partner are shown, with (a) carbon atoms green for 6A6-Fab and (b) carbon atoms pink for TF.

FIG. 8 The interactions of D3H44-Fab CDRs in complex with 6A6-Fab (left side of (a)-(f) view) and TF (right side of (a)-(f) view). Dashed lines indicate hydrogen bonds; large spheres indicate water molecules; small spheres indicate oxygen atoms (some solvent atoms have been removed for clarity). Protein backbone atoms are depicted as tubes. See also, FIG. 8 of J. Mol. Bol. (2003) 331:433-446, 440 for color reproduction where the interactions of D3H44-Fab CDRs (grey) in complex with 6A6-Fab (green) and TF (pink) is depicted and the colored circles on the tubes denote carbonyl oxygen atoms (red) or amide nitrogen atoms (blue).

FIG. 9 Orientation of beta-sheets from 6A6-Fab and TF (both depicted as ribbon structures) with respect to D3H44-Fab (globular surface). See also, FIG. 9 of J. Mol. Bol. (2003) 331:433-446, 441 for color reproduction wherein 6A6-Fab is depicted in green, tissue factor is depicted in pink, and D3H44-Fab is the grey globular structure.

FIG. 10 An amino acid and nucleic acid sequence encoding a 6A6Ch-Fab depicted in (A) and continued in (B).

FIG. 11 (A) A nucleic acid sequence encoding a heavy chain variable, region of a 6A6 antibody. (B) An amino acid sequence of a heavy chain variable region of a 6A6 antibody. The amino acids are numbered according to the Kabat numbering system.

FIG. 12 (A) A nucleic acid sequence encoding a light chain variable region of a 6A6 antibody. (B) An amino acid sequence of a light chain variable region of a 6A6 antibody. The amino acids are numbered according to the Kabat numbering system.

FIG. 13 An amino acid sequence coding for (A) a variable heavy chain region and (B) a variable light chain region of a D3H44 antibody. The amino acids are numbered according to the Kabat numbering system.

FIG. 14 Detection of anti-D3H44 antibodies in chimpanzees. Chimpanzees were treated with D3H44-F(ab′)₂ antibody. Plasma (citrated) samples were prepared from blood drawn before treatment with D3H44-F(ab′)₂ antibody (“pre-treatment”), on day 21 and on day 43 post-treatment and analyzed for anti-D3H44 antibodies by competitive ELISA assay. 6A6 antibody or D3 antibody (control) were incubated with chimpanzee plasma and then the mixture was added to plates coated with D3H44-F(ab′)₂ antibody. Bound chimpanzee antibodies were detected using anti-human Fc antibodies conjugated to horse radish peroxidase (HRP).

### DETAILED DESCRIPTION

The terms “tissue factor” or “tissue factor protein” and “mammalian tissue factor protein” are used to refer to a polypeptide having an amino acid sequence corresponding to a naturally occurring mammalian tissue factor (e.g., U.S. Pat. No. 6,274,142; Fisher et al., [1987] Thromb. Res. 48:89-99; Morrissey et al., [1987] Cell 50:129-135). Naturally occurring TF includes human species as well as other animal species such as rabbit, rat, porcine, non-human primate, equine, murine, and ovine tissue factor (see, for example, Hartzell et al., (1989) Mol. Cell. Biol., 9:2567-2573; Andrews et al., (1991) Gene, 98:265-269; and Takayenik et al., (1991) Biochem. Biophys. Res. Comm., 181:1145-1150). The amino acid sequence of other mammalian tissue factor proteins are generally known or obtainable through conventional techniques.

The term “soluble tissue factor” or “sTF” refers to the amino acid sequence encoding an extracellular domain of tissue factor without the transmembrane and cytoplasmic domainsl. The amino acid sequence of the extracellular domains of tissue factor proteins are generally known or obtainable through conventional techniques. In one one embodiment, a soluble form of human tissue factor is residues 1-219 of human tissue factor.

The term “inactive tissue factor” or “iTF” according to this invention is a tissue factor that has been mutated so that it has substantially less procoagulant activity compared to a soluble tissue factor, but it can still bind to an anti-tissue factor antibody. In one embodiment, iTF is a soluble tissue factor that has been mutated at its Factor VIIa binding site so that Factor VIIa binding is decreased. This can be achieved by mutating one or more residues of interaction between tissue factor and Factor VIIa. See, for example, Kelley et al., Biochemistry (1995) 34:10383-10392; Banner et al., Nature (1996) 380:41-46 for residues of interaction between Factor VIIa and tissue factor. In another embodiment, the iTF is a soluble human TF having a mutation selected from the group consisting of K20, D44, Q37, W45, D58, D58, Y94, F76, E91, Y94 and R74. In one embodiment, the iTF binds to the anti-tissue factor antibody with an affinity that is similar to a soluble tissue factor.

The region of tissue factor that interacts with Factor X is known and includes a C-terminal portion of the extracellular domain of tissue factor. According to one embodiment, the region comprises at least residues numbered 150-204 of human tissue factor.

The term “anti-tissue factor antibody” or “ATF” as used here refers to an antibody that specifically binds to a tissue factor.

The term “anti- anti-tissue factor antibody,” “AATF,” “AATF antibody,” or “AATF polypeptide” as used herein refers to an antibody or polypeptide that specifically binds to a variable region of an antibody that specifically binds to the extracellular domain of a tissue factor.

The term “antibody” (Ab) as used herein includes monoclonal antibodies, polyclonal antibodies, multispecific antibodies (e.g. bispecific antibodies), and antibody fragments, so long as they exhibit the desired biological activity The term “immunoglobulin” (Ig) is used interchangeably with “antibody” herein.

The term “antibody” is used in the broadest sense and specifically covers, for example, single monoclonal antibodies, multispecific antibodies (such as bispecific antibodies), antibody compositions with polyepitopic specificity, polyclonal antibodies, single chain antibodies, and fragments of antibodies (see below) as long as they specifically bind a native polypeptide and/or exhibit a biological activity or immnunological activity of this invention. The phrase “functional fragment or analog” of an antibody is a compound having a qualitative biological activity in common with an antibody to which it is being referred. For example, a functional fragment or analog of an antibody of this invention can be one which can specifically bind to a variable region of a anti-tissue factor antibody. In one embodiment, the antibody can prevent or substantially reduce the ability of the anti-tissue factor antibody to inhibit blood coagulation. The term “immunoglobulin” (Ig) is used interchangeably with “antibody” herein.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35 (H1), 50-65 (H2) and 95-102 (H3) in the V_{H}; Kabat et al., Sequences of Proteins of Immunological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they can be synthesized uncontaminated by other antibodies. The modifier “monoclonal” is not to be construed as requiring production of the antibody by any particular method. For example, the monoclonal antibodies useful in the present invention can be prepared by the hybridoma methodology first described by Kohler et al., Nature, 256:495 (1975), or can be made using recombinant DNA methods in bacterial, eukaryotic animal or plant cells (see, e.g., U.S. Pat. No. 4,816,567). The “monoclonal antibodies” can also be isolated from phage antibody libraries using the techniques described in Clackson et al., Nature, 352:624-628 (1991), Marks et al., J. Mol. Biol., 222:581-597 (1991), and the Examples below, for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C_{L} and at least heavy chain constant domains, C_{H}1, C_{H}2 and C_{H}3. The constant domains can be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

The expression “linear antibodies” generally refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an-antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRUB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fe), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

A “variant” means a sequence having at least about 80% amino acid sequence identity with a full-length sequence to which it is being compared. Such variants include, for instance, the polypeptides or antibodies of this invention wherein one or more amino acid residues are added, or deleted, at the N— or C-terminus of the full-length native amino acid sequence. Ordinarily, a variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity; to a the sequence against which it is being compared. Ordinarily, variants are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430,440, 450, 460, 470, 480, 490,500, 510, 520, 530, 540, 550, 560, 570, 580, 590, 600 amino acids in length, or more. Optionally, a variant will have no more than one conservative amino acid substitution as compared to the sequence against which is being compared, alternatively no more than 2, 3, 4, 5, 6, 7, 8, 9, or 10 conservative amino acid substitution as compared to the sequence against which is being compared. Preferably, a variant of an AATF antibody of this invention will specifically bind to the variable region of an ATF antibody.

“Percent (%) amino acid sequence identity” with respect to the polypeptide sequences identified herein is defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Gehentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 1 and 2 demonstrate how to calculate the % amino acid sequence identity of the amino acid sequence designated “Comparison Protein” to the amino acid sequence designated “A”, wherein “A” represents the amino acid sequence of a hypothetical polypeptide of interest, “Comparison Protein” represents the amino acid sequence of a polypeptide against which the “A” polypeptide of interest is being compared, and “X, “Y” and “Z” each represent different hypothetical amino acid residues. Unless specifically stated otherwise, all % amino acid sequence identity values used herein are obtained as described in the immediately preceding paragraph using the ALIGN-2 computer program.

TABLE 1

| | | |
|---|---|---|
| “A” | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of a polypeptide or antibody of this invention) = 5 divided by 15 = 33.3%

TABLE 2

| | | |
|---|---|---|
| “A” | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the “A” polypeptide) = 5 divided by 10 = 50%

In other embodiments, polynucleotides that are included in this invention are nucleic acid molecules that encode an antibody or polypeptide which are capable of hybridizing, preferably under stringent hybridization and wash conditions, to nucleotide sequences encoding an antibody or polypeptide of this invention as disclosed herein.

“Isolated,” when used to describe the polypeptides or antibodies of this invention, means a polypeptide or antibody that has been identified and separated and/or recovered from a component of its natural environment. Contaminant components of its natural environment are materials that would typically interfere with diagnostic or therapeutic uses for the polypeptide or antibody and can include enzymes, hormones, and other proteinaceous or non-proteinaceous solutes. In one embodiment, the polypeptides or antibodies can be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptides or antibodies include polypeptides and antibodies in situ within recombinant cells, since at least one component of the polypeptide or antibody natural environment will not be present. Ordinarily, however, an isolated polypeptide or antibody will be prepared by at least one purification step.

An “isolated” nucleic acid molecule of this invention is a nucleic acid molecule that is identified and separated from at least one contaminant nucleic acid molecule with which it is ordinarily associated within a cell in which it can be replicated. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 C; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42 C, with a 10 minute wash at 42C in 0.2×SSC (sodium chloride/sodium citrate) followed by a 10 minute high-stringency wash consisting of 0.1×SSC containing EDTA at 55 C.

“Moderately stringent conditions” can be identified as described by Sambrook et al., Molecular Cloning: A Laboratory Manual, New York: Cold Spring Harbor Press, 1989, and-include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50C. The skilled artisan will recognize how to adjust the temperature, ionic strength, etc. as necessary to accommodate factors such as probe length and the like.

A “procoagulant” is an agent that causes, stimulates, accelerates or facilitates blood coagulation. For example, procoagulants according to this invention include clotting factors (e.g., VIII, IX, X, prothrombin, tissue factor), plasminogen activator inhibitors (e.g., PAI-1), plasmin inhibitors (e.g., aprotinin), vitamin K, plasma fractions, desmopressin acetate, prothrombin concentrates, and platelet concentrates. Many procoagulants are known in the art and are commercially available. According to one embodiment of this invention, the procoagulant is a recombinant factor VIIa (e.g., NovoSeven®, Novo Nordisk A/S, Denmark).

The term “anticoagulant” activity is used to refer to the ability of a substance to prevent, inhibit or prolong blood coagulation in an in vitro or in vivo assay of blood coagulation.

Blood coagulation assays are known in the art and include, for example, prothrombin time assays such as those described in the examples herein, the human ex vivo thrombosis model described by Kirchhofer et al., Arterioscler. Thromb. Vasc. Biol. 15, 1098-1106 (1995); and Kirchhofer et al., J. Clin. Invest. 93, 2073-2083 (1994), and assays based on the measurement of Factor X activation in human plasma, and in the examples as described in the present application.

The term “epitope” is used to refer to binding sites for (monoclonal or polyclonal) antibodies on protein antigens.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in a number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term amino acid or amino acid residue, as used herein, refers to naturally occurring L amino acids or to D amino acids as described further below with respect to variants. The commonly used one- and three-letter abbreviations for amino acids are used herein (Bruce Alberts et al., Molecular Biology of the Cell, Garland Publishing, Inc., New York (3d ed. 1994)).

By “biological characteristics” is meant the in vitro and/or in vivo activities of the antibody selected from the group consisting of the ability to specifically bind to a variable region of an anti-tissue factor antibody; to specifically bind to a CDR of an anti-tissue factor antibody and to inhibit the anticoagulant activity of an anti-tissue factor antibody (observed by, e.g., macromolecular substrate activation assays, the whole blood clotting assays or the prothrombin assays known in the art and as described in the Examples). According to one embodiment, the antibody preferably binds to substantially the same epitope as the deposited 6A6 antibody. In one embodiment, the antibody has about the same, or greater, binding affinity than the 6A6 antibody produced the deposited hybridoma cell disclosed herein. In another embodiment, the antibody of this invention has substantially the same, or greater, binding anticoagulant activity than the 6A6 antibody produced the deposited hybridoma cell disclosed herein. To determine whether a monoclonal antibody binds essentially the same epitope as the 6A6 antibodies specifically disclosed (e.g., the antibody produced by the hybridoma cell line having the ATCC Deposit No. PTA-5066), one can, for example, use a competitive ELISA binding assay as described below.

A “hypercoagulable state” is one in which due to an inherited or acquired disorder there is an increased propensity for thrombosis. This state is manifested clinically by either an increase in number of thrombotic events or episodes, thrombosis at an early age, a familial tendency toward thrombosis, and thrombosis at unusual sites. Patients that are susceptible to developing a hypercoagulable state include those having the following history: (1) thrombosis at a young age (age under 50 years); (2) family history of thrombosis; (3) recurrent thrombosis; (4) thrombosis in an unusual site; and (5) pregnancies complicated by frequent miscarriage.

Hypercoagulable states or diseases can be passed onto in family members that inherit particular diseases or abnormalities (e.g., Factor V Leiden Deficiency, Homocystinuria or Hyperhomocysteinemia, Antithrombin III deficiency, Protein C Deficiency, Protein S Deficiency, increased Factor VIII, Fibrinolysis, and Dysfibrinogenemia). Hypercoagulable states can be acquired as a result of other conditions (e.g., pregnancy, estrogen consumption (oral contraceptives, estrogen replacement therapy, tamoxifen), surgery, trauma, infection, bites of poisonous snakes, acute liver disease, sepsis, malignancy (cancer in idiopathic hypercoagulability), myeloproliferative disorder, hyperlipideria, homocystinuria, systemic lupus erythematosus, burns, renal disease, eclampsia, heat stroke, antiphospholipid antibodies, nephrotic syndrome, neoplasms). Manipulation of body fluids can also result in an undesirable thrombus, particularly in blood transfusions or fluid sampling, as well as procedures involving extracorporeal circulation (e.g., cardiopulmonary bypass surgery) and dialysis.

The terms “cell proliferative disorder” and “proliferative disorder” refer to disorders that are associated with some degree of abnormal cell proliferation. In one embodiment, the cell proliferative disorder is cancer.

The terms “cancer” and “cancerous” refer to or describe the pathological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

Inflammatory diseases according to this invention include, for example, joint inflammation, including arthritis, rheumatoid arthritis and other arthritic conditions such as rheumatoid spondylitis, gouty arthritis, traumatic arthritis, rubella arthritis, psoriatic arthritis, osteoarthritis. acute synovitis, autoimmune diabetes, autoimmune encephalomyelitis, collitis, atherosclerosis, peripheral vascular disease, cardiovascular disease, multiple sclerosis, asthma, psoriasis restenosis, myocarditis, inflammatory bowel disease, pelvic inflammatory disease, scleroderma and systemis lupus erythematosus.

“Mammal” for purposes of the treatment of, alleviating the symptoms of or diagnosis of a hypercoagulable state, illness or disease according to this invention refers to any animal classified as a mammal (aka “patient”), including humans, domestic and farm animals, and zoo, sports, or pet animals, such as dogs, cats, cattle, horses, sheep, pigs, goats, rabbits, chimps, baboons, monkeys, etc. Preferably, the mammal is human.

The methods of this invention are suited to treat, e.g., thrombosis. Thrombotic conditions associated with hypercoagulable states include acute disseminated intravascular coagulation, septic shock, coronary thrombosis, organ transplant rejection, and deep vein thrombosis. Samples can be suitably obtained from a mammal suffering from or suspected of suffering from thrombosis, preferably restenosis, associated with, e.g., an invasive medical procedure such as cardiopulmonary bypass surgery; a heart ailment such as myocardial infarction, cardiomyopathy, valvular heart disease, unstable angina, or artrial fibrillation associated with embolization; a coagulopathy including disseminated intravascular coagulation, pulmonary embolism (e.g., atrial fibrillation with embolization), deployment of an implementation such as a stent or catheter; shock (e.g., septic shock syndrome), vascular trauma, liver disease, heat stroke, malignancies (e.g., pancreatic, ovarian, or small lung cell carcinoma), lupus, eclampsia, perivascular occlusive disease, and renal disease. Exogenous administration of tissue factor can lead to disseminated intravascular coagulation (DIC). Prentice, C. R., Clin. Haematol. 14(2), 413442 (1985).

Various tag polypeptides and their respective antibodies are well known in the art. Tagged polypeptides and antibodies of this invention are contemplated. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. The FLAG-peptide [Hopp et al., BioTechnology, 6:1204-1210 (1988)] is recognized by an anti-FLAG M2 monoclonal antibody (Eastman Kodak Co., New Haven, Conn.). Purification of a protein containing the FLAG peptide can be performed by immunoaffinity chromatography using an affinity matrix comprising the anti-FLAG M2 monoclonal antibody covalently attached to agarose (Eastman Kodak Co., New Haven, Conn.). Other tag polypeptides include the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin; or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days, later the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

(ii) Monoclonal Antibodies

Monoclonal antibodies may be made using the hybridoma method first described by Kohler et al., Nature, 256:495 (1975), or may be made by recombinant DNA methods (U.S. Pat. No 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Types of useful fusion partner myeloma cells include are those that fuse efficiently, support stable high-level production of antibody by the selected antibody-producing cells, and are sensitive to a selective medium that selects against the unfused parental cells. In one embodiment, myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications,. pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal e.g, by i.p injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as one source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified, for example, by substituting human heavy chain and light chain constant domain (C_{H} and C_{L}) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide. The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunuol., 151:2623 (1993)).

It is further important that antibodies be humanized with retention of high binding affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et. al., Year in Immuno., 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 f19901) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

(v) Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However; these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Fab and F(ab′)₂ with increased in vivo half-life is described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No.5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the anti-tissue factor antibody. Other such antibodies may combine an anti-tissue factor antibody binding site with a binding site for another protein. Alternatively, an anti-tissue factor antibody arm may be combined with an arm which binds to a triggering molecule on a leukocyte such as a T-cell receptor molecule (e.g. CD3), or Fc receptors for IgG (FçγR), such as FçγRI (CD64), FcγRII (CD32) and FcγRIII (CD 16), so as to focus and localize cellular defense mechanisms to the -expressing cell. Bispecific antibodies can also be used to localize cytotoxic agents to cells which express tissue factor that have been bound with an ATF antibody. These antibodies possess an anti-tissue factor antibody-binding arm and an arm which binds the cytotoxic agent (e.g. saporin, anti-interferon-, vinca alkaloid, ricin A chain, methotrexate or radioactive isotope hapten). Bispecific antibodies can be prepared as full length antibodies or antibody fragments (e.g. F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FçγRI antibody. A bispecific anti-ErbB2/Fç antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is one embodiment of this invention to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. In one embodiment, the interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 0308936). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med, 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (199 1)

(vii) Other Amino Acid Sequence Modifications

Amino acid sequence modification(s) of the antibodies and polypeptides of this invention are contemplated. For example, it may be desirable to improve the binding affinity and/or other biological properties of the antibody. Amino acid sequence variants of the antibodies and polypeptides of this invention are prepared by introducing appropriate nucleotide changes into the nucleic acid encoding them, or by peptide synthesis. Such modifications include, for example, deletions from, and/or insertions into and/or substitutions of, residues within the amino acid sequences of the antibodies and polypeptides of this invention. Any combination of deletion, insertion, and substitution is made to arrive at the final construct, provided that the final construct possesses the desired characteristics. The amino acid changes also may alter post-translational processes of the antibodies and polypeptides of this invention, such as changing the number or position of glycosylation sites.

A useful method for identification of certain residues or regions of the antibodies and polypeptides of this invention that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen (e.g., anti-tissue factor antibody). Those amino acid locations demonstrating functional sensitivity to the substitutions then are refined by introducing further or other variants at, or for, the sites of substitution. Thus, while the site for introducing-an amino acid sequence variation is predetermined, the nature of the mutation per se need not be predetermined. For example, to analyze the performance of a mutation at a given site, ala scanning or random mutagenesis is conducted at the target codon or region and the expressed antibody or polypeptide variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody or polypeptide with an N-terminal methionyl residue or the antibody fused to a cytotoxic polypeptide. Other insertional variants of the anti-AATF antibody polypeptide include the fusion to the N— or C-terminus of the anti-AATF antibody to an enzyme (e.g. for ADEPT) or a polypeptide which increases the serum half-life of the antibody.

Another type of variant is an amino acid substitution variant. These variants have at least one amino acid residue in the antibody or polypeptide molecule replaced by a different residue. The sites of greatest interest for substitutional mutagenesis include the hypervariable regions, but FR alterations are also contemplated. Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened.

TABLE 3

Amino Acid Substitutions

| | | |
|---|---|---|
| Original | Exemplary | Preferred |

| | | |
|---|---|---|
| Residue | Substitutions | Substitutions |

| | | |
|---|---|---|
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; | ile |
| | ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Substantial modifications in the biological properties of the polypeptide and antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the antibody or polypeptide also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and anti-tissue factor antibody. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the antibodies and polypeptides of this invention are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of an antibody or polypeptide of this invention.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(viii) Screening for Antibodies with the Desired Properties

Techniques for generating antibodies have been described above. One may further select antibodies with certain biological characteristics, as desired. For example, antibody binding studies can be carried out using known assay methods, such as competitive binding assays, direct and indirect sandwich assays, and immunoprecipitation assays. Zola, Monoclonal Antibodies: A Manual of Techniques (CRC Press, Inc., 1987), pp. 147-158.

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies can conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody can itself be labeled with a detectable moiety (direct sandwich assays) or can be measured using an anti-immunoglobulin antibody that is labeled with a detectable moiety (indirect sandwich assay). For example, one type of sandwich assay is an ELISA assay, in which case the detectable moiety is an enzyme.

Competitive ELISA assays can be performed to screen polypeptides, antibodies or antagonists for those that specifically bind to an anti-tissue factor antibody, the binding of which can be inhibited by an antibody or polypeptide of this invention. For example, a competitive ELISA assay can be used to determine whether the binding to an anti-tissue factor antibody by the antibody or polypeptide to be tested can be inhibited by the deposited 6A6 antibody or another antibody described herein. First, 96-well Nunc Maxisorp plates can be coated with full length anti-tissue factor antibody (e.g., an antibody containing the variable domain of D3H44) (2 μg/ml in 50 mM carbonate buffer, pH 9.6, 100 μl/well) at 4° C. overnight or at room temperature for 2 hours. The wells can be blocked by adding 150 μl 0.05% BSA in PBS for 1 hour. Next, the wells can be washed with PBS −0.05% Tween20 5 times. Afterwards, varying amounts of the deposited 6A6 antibody or variants with lower ATF binding in ELISA buffer (PBS —0.05% BSA and 0.05% Tween20) and test antibody or polypeptide (labeled, if necessary) are pre-mixed and then added to the wells for 2 hours at room temperature. Then, the wells can be washed with PBS −0.05% Tween20 10 times.

Binding can be quantified by determining the amount of test antibody that bound to the anti-tissue factor antibody. An antibody that does not share essentially the same epitope with 6A6 antibody should not experience decreased binding to tissue factor with increasing concentrations of 6A6 antibody present.

Alternatively or additionally, the antibodies and polypeptides of this invention can be assayed for the desired activity using the macromolecular substrate activation assays, the whole blood clotting assays or the prothrombin assays known in the art and described in the Examples.

(ix) Immunoconjugates

The invention also pertains to therapy with immunoconjugates comprising an antibody conjugated to a cytotoxic agent (cytotoxic agent described above).

Chemotherapeutic agents useful in the generation of such immunoconjugates have been described above.

Conjugates of an antibody and one or more small molecule toxins, such as a calicheamicin, a maytansine (U.S. Pat. No. 5,208,020), a trichothene, and CC1065 are also contemplated herein.

(x) Other Antibody or Polypeptide Modifications

Other modifications of the antibody or polypeptides of this invention are contemplated herein. For example, the antibody or polypeptide can be linked to one of a variety of nonproteinaceous polymers, e.g., polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The antibodies and polypeptides of this invention can also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody or polypeptide can be prepared by methods known in the art, such as described in Epstein et al., Proc. Natl. Acad. Sci. USA, 82:3688(1985); Hwang et al., Proc. Natl Acad. Sci. USA, 77:4030(1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257:286-288 (1982) via a disulfide interchange reaction. A chemotherapeutic agent is optionally contained within the liposome. See Gabizon et al. J. National Cancer Inst. 81(19)1484 (1989).

III. Pharmaceutical Formulations

Therapeutic formulations of the antibodies, polypeptides and nucleic acid molecules used in accordance with the present invention are prepared for storage by mixing those that have the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g. Zn-protein complexes); and/or non-ionic surfactants such as TWEEN™, PLURONICS™ or polyethylene glycol (PEG). The formulation preferably comprises the antibody, polypeptide or nucleic acid molecule at a concentration of between 5-200 mg/ml, preferably between 10-100 mg/ml.

The formulation herein may also contain more than one active compound (therapeutic agent) as necessary for the particular indication being treated, preferably those with complementary activities that do not adversely affect each other. Alternatively, or additionally, the composition may further comprise a chemotherapeutic agent, procoagulant, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed.(1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid.

The formulations to be used for in vivo administration must be sterile. This is readily accomplished by filtration through sterile filtration membranes.

IV. Treatment with the Antibodies, Polypeptides and Nucleic Acid Molecules

For the prevention or treatment of disease, the appropriate dosage of compositions of this invention will depend on the type of disease to be treated, as defined above, the severity and course of the disease, whether the antibody or polypeptide is administered for preventive or therapeutic purposes, previous therapy, the patient's clinical history and response to the antibody of polypeptide, and the discretion of the attending physician. The antibody or polypeptide is suitably administered to the patient at one time or over a series of treatments. Depending on the type and severity of the disease, about 1 μg/kg to 15 mg/kg (e.g. 0.1-10 mg/kg) of antibody, polypeptide or-nucleic acid molecule is an initial candidate dosage for administration to the patient, whether, for example, by one or more separate administrations, or by continuous infusion. A typical daily dosage might range from about 1 μg/kg to 100 mg/kg or more,.depending on the factors mentioned above. For repeated administrations over several days or longer, depending on the condition, the treatment is sustained until a desired suppression of disease symptoms occurs. The disease symptoms and parameters for assessing improvement and the progress of this therapy can be readily monitored by conventional methods and assays known to-the physician or other persons of skill in the art.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The current in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:44294432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

V. Articles of Manufacture

In another embodiment of the invention, an article of manufacture containing materials useful for the treatment of a hypercoagulable state is provided. The article of manufacture comprises a (1) container and a (2) label or package insert comprising instructions on how to use the antibodies and polypeptides of this invention. Another embodiment of the invention is an article of manufacture containing materials useful for the treatment of cancer. Suitable containers include, for example, bottles, vials, syringes, etc. The containers can be formed from a variety of materials such as glass or plastic. The container holds a composition which is effective for treating the condition and can have a sterile access port (for example the container may be an intravenous solution bag or a vial having a stopper pierceable by a hypodermic injection needle). At least one “active agent” in the composition is an antibody, polypeptide or nucleic acid molecule of this invention. The label or package insert indicates that the composition is used for treating a hypercoagulabe state. The label or package insert will further comprise instructions for administering the antibody composition to the patient.

Moreover, the article of manufacture can comprise (a) a first container with a composition contained therein, wherein the composition comprises an ATF antibody which binds tissue factor and inhibits the coagulant activity of tissue factor; and (b) a second container with a composition contained therein, wherein the composition comprises a second antibody or polypeptide of this invention (ant AATF antibody or polypeptide) that binds the ATF antibody and inhibits the ATF antibody activity. The article of manufacture in this embodiment of the invention can further-comprises a package insert indicating that the first and second compositions can be used to treat a hypercoagulable state or an inflammatory disease. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for purification or immunoprecipitation of anti-tissue factor antibodies from cell culture, for performing macromolecular substrate activation assays, the whole blood clotting assays or the prothrombin assays. For isolation and purification of anti-tissue factor antibodies, the kit can contain an antibody or polypeptide of this invention coupled to beads (e.g., sepharose beads). Kits can be provided which contain the antibodies for detection and quantitation of anti-tissue factor antibodies in vitro, e.g. in an ELISA or a Western blot. As with the article of manufacture, the kit comprises a container and a label or package insert on or associated with the container. The container holds a composition comprising at least one antibody, polypeptide or nucleic acid molecule of the invention. Additional containers may be included that contain, e.g., diluents and buffers, control antibodies. The label or package insert may provide a description of the composition as well as instructions for the intended in vitro or diagnostic use.

VI. Deposit of Materials

The hybridoma cell line, 6A6.3E11.2E12 (deposit number PTA-5066), that produces a 6A6 antibody was deposited under the terms of the Budapest Treaty with the American Type Culture Collection (ATCC), 10801 University Blvd., Manassas, Va. 20110-2209, USA on Mar. 20, 2003.

VII. Non-Therapeutic Utility

The antibodies and polypeptides of the invention also have non-therapeutic applications, for example, for diagnostic purposes to help determine whether a patient receiving treatments of an anti-tissue factor antibody has developed antibodies can block the therapeutic use of the anti-tissue factor antibody. For example, the antibodies or polypeptides of this invention can be useful in a competitive ELISA assay to examine such antibodies in the sera or plasma of a treated patient. The anti-tissue factor antibody being administered as a therapeutic to the patient can be used as the substrate having the epitope for which the antibodies of this invention and the antibodies of the patient compete. See, e.g., Example 13, below. Additionally, the antibodies or polypeptides of this invention can be used in another functional assay such as a macromolecular substrate assay, prothrombin assay or whole blood clotting assay in combination with an anti-tissue factor antibody to which it binds. Articles of manufacture or kits comprising the antibodies or polypeptides of this invention, optionally including an anti-tissue factor antibody to which the AATF antibodies or polypeptides bind, for use in determining the presence and/or amount of AATF antibody in a mammal being treated with ATF is contemplated.

The antibodies and polypeptides of this invention can also be used to purify ATF antibodies. For example, the antibodies and polypeptides of this invention can be bound to a solid support for affinity chromatography purification.

All publications (including patents and patent applications) cited herein are hereby incorporated in their entirety by reference, including U.S. Provisional Application Nos. 60/474,534, filed May 30, 2003 and 60/476,025, filed Jun. 4, 2003 and Eigenbrot, C., et al., (2003) J. Mol. Biol. 331:433-446.

The deposit herein was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit The deposit will be made available by ATCC under the terms of the Budapest Treaty, and subject to an agreement between Genentech, Inc. and ATCC, which assures permanent and unrestricted availability of the progeny of the culture of the deposits to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. 122 and the Commissioner's rules pursuant to thereto (including 37 C.F.R. 1.14 with particular reference to 886 OG 638).

The assignee of the present application has agreed that if a culture of the materials on deposits should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, Va. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Materials

Fatty acid-free BSA was purchased from Calbiochem (La Jolla, Calif.). Human recombinant F.VIIa was a gift from Mark O'Connell (Genentech, Inc., South San Francisco, Calif.). F.X and F.IX were obtained from Haematologic Technologies Inc. (Essex Junction, Vt.). F.Xa chromogenic substrate S2765 from Diapharma Group Inc. (Columbus, Ohio) and F.IXa chromogenic substrate #299 from American Diagnostica (Greenwich, Conn.). Truncated transmembrane tissue factor comprising residues 1-243 (TF₁₋₂₄₃) was produced and relipidated (relTF₁₋₂₄₃) as described (Presta, L. et al. (2001), supra). Soluble TF comprising residues 1-219 (sTF₁₋₂₁₉) was prepared as described (Kelley, R. F. et al. Biochemistry 34, 10383-103892 (1995)). Antibodies that recognize tissue factor, 5G6, D3H44-Fab, D3H44-F(ab′)₂, D3H44-IgG4b, were produced as described recently (Presta, L. et al.(2001), supra; Kirchhofer, et al., (2000) Thromb. Haemost. 84:1072-81). A humanized anti-HER2 antibody (the Herceptin® antibody) was obtained from Genentech.

### Example 2

### Generation of 6A6 Murine Monoclonal Antibody

6A6 monoclonal antibody was generated by injecting 5 μg of D3H44 Fab in the footpads of BALB/c mice (Charles River Laboratories, Wilmington, Del.) eleven times. Lymph nodes from mice with high antibody titers to immobilized D3H44-Fab were fused with mouse myeloma cells (X63.Ag8.653; American Type Culture Collection, Rockville, Md.) as described previously (Hongo J.-A. S. et al. Hybridoma, 14, 253-260 (1995)). Hybridoma cells were analysed for binding to D3H44-Fab and the Herceptin® antibody (Carter P. et al., Proc. Natl. Acad. Sci. U.S.A. 89, 4285-4289 (1992)) in ELISA-type assays. The Herceptin® antibody has the same constant region as D3H44 and only differs in the variable domains. Cells producing antibodies specific to D3H44 were then cloned by limiting dilution. This produced clone 6A6 which specifically bound D3H44-Fab. A hybridoma cell line, 6A6.3E11.2E12 (deposit number PTA-5066), that produces a 6A6 antibody was deposited under the terms of the Budapest Treaty with the American Type Culture Collection (ATCC), 10801 University Blvd., Manassas, Va. 20110-2209, USA on Mar. 20, 2003.

### Example 3

### Cloning of 6A6 Variable Domainand Construction of Chimeric muV/huC Fab (6A6Ch-Fab)

Monoclonal antibody 6A6 was purified from ascites (for heavy chain) or hybridoma supernatant (for light chain). The N-terminal sequences for the heavy chain, EVQLQQSGPELVKPGASVKISCKAS (SEQ ID NO:8), and for the light chain, QAVVTQESALTSMPG (SEQ ID NO:9), were determined and used for primer design. Total RNA was isolated from the 6A6 hybridoma cell line using the RNeasy kit protocol for animal cells (Qiagen). cDNA for the heavy chain was made and amplified using the Qiagen One Step RT-PCR kit (Qiagen) with a single specific heavy chain forward primer, 5′-TCACOCGTACGCTGAGGTYCAGCTGCARCA-3′(SEQ ID NO:10), and three different heavy chain reverse primers, 5′-ATGGGCCCGTCGTTTTGGCTGAGGAGACDGTGASMRDRGT-3′(SEQ ID NO: 11), 5′-ATGGGCCCGTCGTTTTGGCTGCAGAGACDGTGA-3′(SEQ ID NO: 12), and 5′-ATGGGCCCGTCGTTTTGGCTGAGGAGACDGTGA-3′(SEQ ID NO:13). Using a Perkin Elmer 9600 Thermocycler, reverse transcription took place at 50° C. for 30 minutes followed by: HotStarTaq DNA Polymerase activation at 95° C. for 15 minutes; 35 cycles of (94° C. for 1 minute, 52° C. for 1 minute 72° C. for 1 minute), 72° C. for 10 minutes, and then held at 4° C. PCR products from all three heavy chain reverse primer reactions were combined. Following purification with QIAquick spin columns (Qiagen), the heavy chain PCR product and a previously described F(ab) chimeric expression plasmid [Presta L. G. et al., Cancer Res. 57, 4593-4599(1997)] were digested sequentially with ApaI and MluI (New England BioLabs). The vegf4chim plasmid encodes a human C_{L} region. Thus, the 6A6 mouse V_{L} sequence is ligated in frame with a human C_{L} sequence. Products were separated on a 1.2% agarose gel and appropriate fragments were isolated and purified with the QIAquick Gel Extraction Kit (Qiagen). The 6A6 murine heavy chain variable region fragment was cloned into the Fab chimeric expression plasmid using T4 DNA Ligase (New England BioLabs), and cells (Max Efficiency DH5α Competent Cells, Gibco BRL) were transformed with the resulting plasmid. After-sequencing, clones with the proper heavy chain variable region were amplified (Qiagen midiprep) for subsequent light chain variable region insertion.

As with the heavy chain, 6A6 light chain cDNA was made and amplified using a specific light chain forward primer, 5′-CAAATGCATACGCTCAGGCTGTTGTGACTCAG-3′(SEQ ID NO:14), and light chain reverse primer, 5′-GCCACGGTCCGTAGGACAGTSASTFTGGTTCC-3′(SEQ ID NO:15). The 6A6 light chain variable region was then cloned into the Fab chimeric expression plasmid containing 6A6 heavy chain variable region using RsrII, NsiI, and T4 DNA Ligase (New England BioLabs). The vegf4chim plasmid encodes a human C_{H}1 region. Thus, the 6A6 mouse V_{H} sequence is ligated in frame with a human C_{H}1 sequence. The final product, plasmid pxD3 (FIG. 2), was used to transform cells, and clones were sequenced to verify the presence of 6A6 heavy and light chain variable regions.

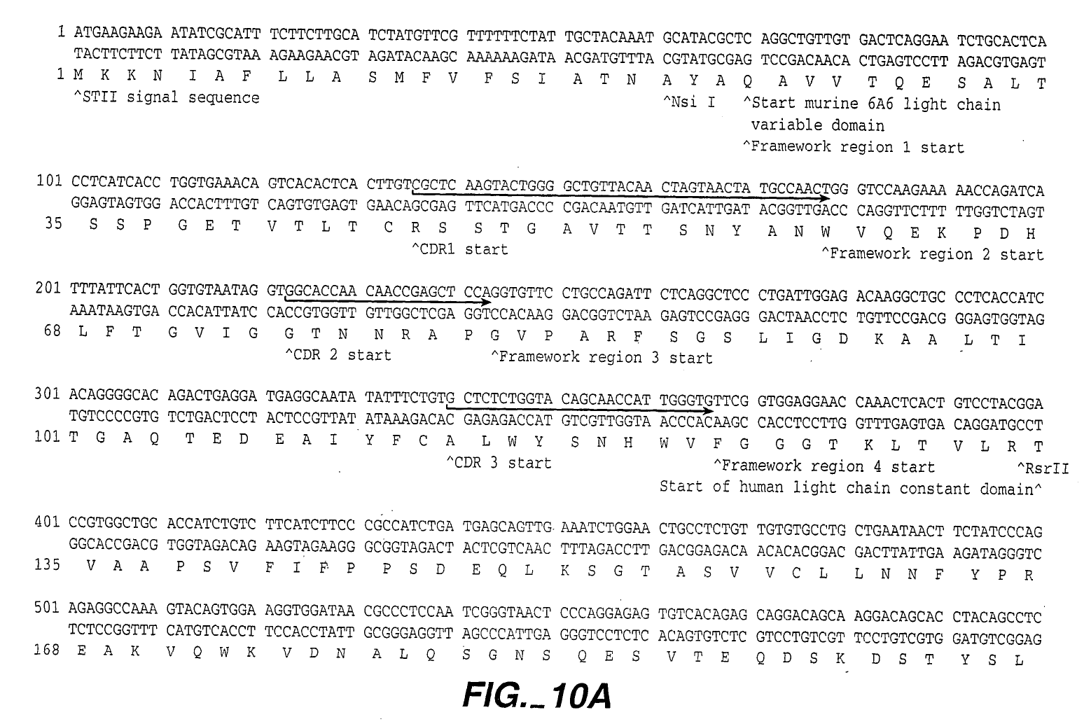

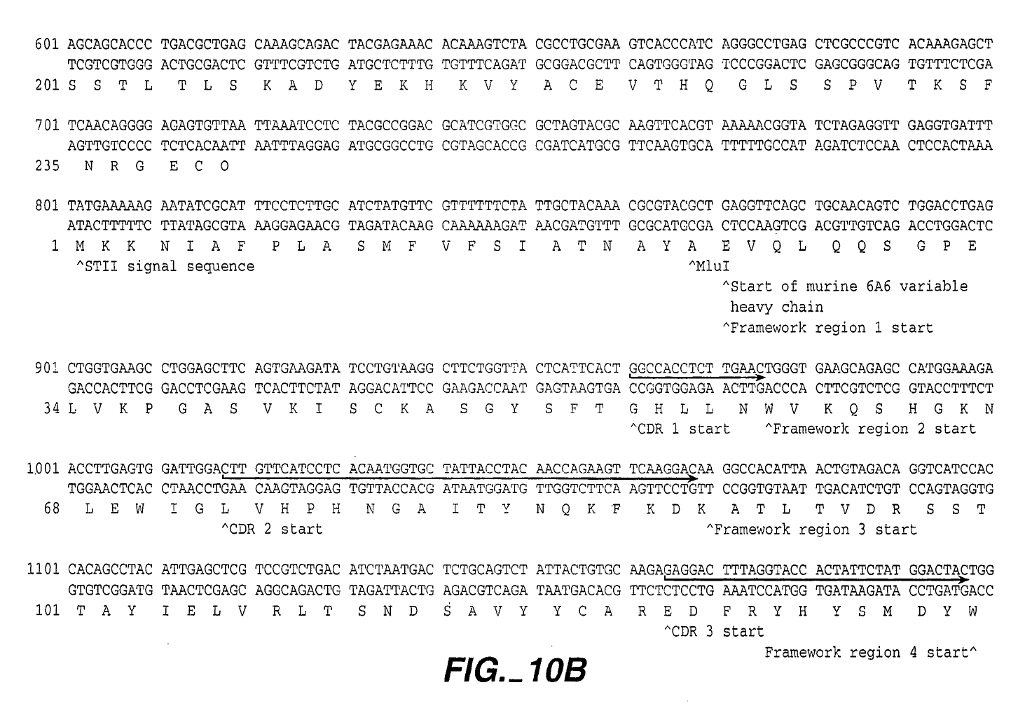

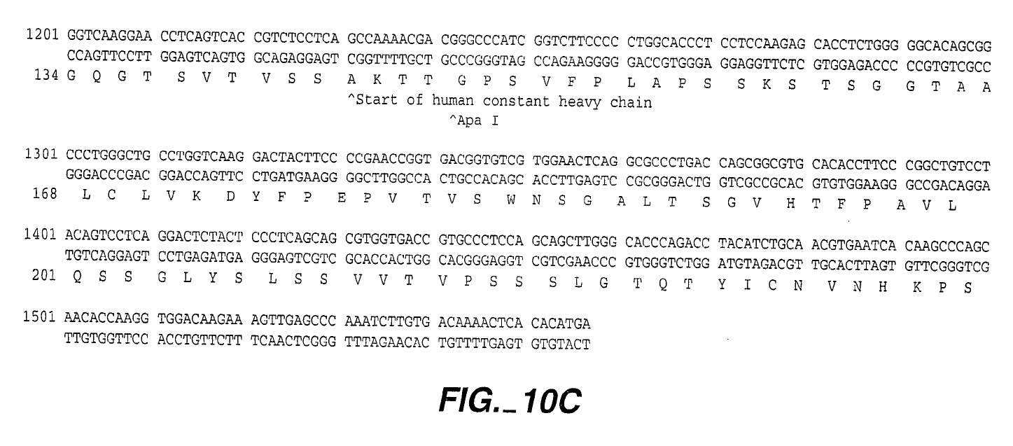

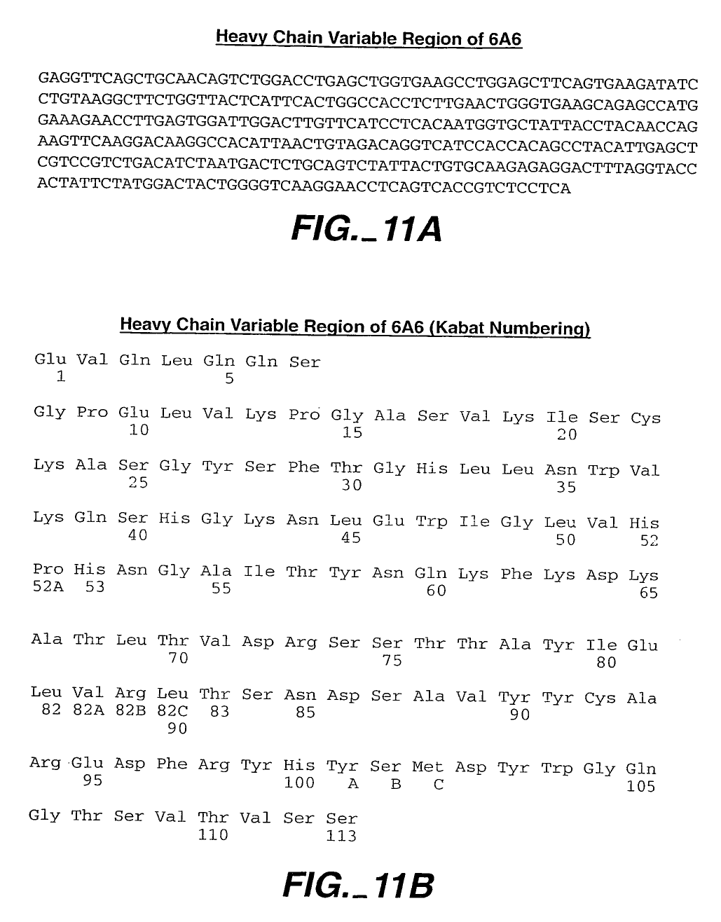

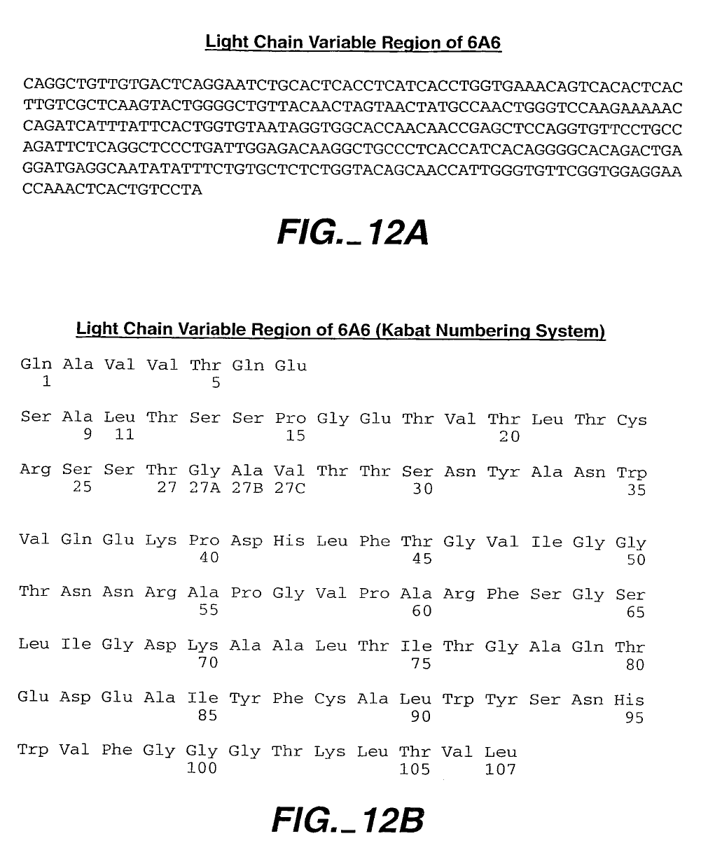

A nucleic acid sequence encoding the heavy chain variable region and the light chain variable region of a 6A6 antibody is shown in FIG. 10 and FIG. 11, respectively. An amino acid sequence and nucleic acid sequence encoding a 6A6Ch-Fab is shown In FIG. 12A-C.

### Example 4

### Expression and Purification of Chimeric muV/huC Fab (6A6Ch-Fab)

Transfection of E. coli with 6A6Ch-Fab plasmid (pxD3) and E. coli fermentation was carried out by methods described previously (Simmons, et al. J. Immul. Methods 263 (2002), 133-147). Fermentation paste was diluted in PBS containing protease inhibitor (Complete protease inhibitor, Roche Diagnostics, Mannhein, Germany) and then lysed using a Microfluidizer (Model 110F, Microfluidics Corp., Newton, Mass.). The lysate was clarified by centrifugation at 4300×g for 30 min and then adjusted to pH 3.8. After centrifugation to remove the precipitate, the supernatant was collected and filtered (0.45 micron filter). After a 10-fold concentration step (Centriprep YM-30, Millipore Corp. Bedford, Mass.) the concentrated lysate was applied onto a D3H44-IgG4b antibody affinity column (Presta, L. et al. Thromb. Haemost (2001), supra), which was prepared by use of the AminoLink Plus Kit (Pierce, Rockford Ill.). The coupling efficiency was about 90% resulting in approximately 7 mg of D3H44-IgG4/ml resin. After loading the lysate, the column was washed with PBS and the bound 6A6Ch was eluted with 0.1M glycine, pH 2.8. The eluted fractions were immediately neutralized with 2 M Tris-HCl, pH 9.0. The 6A6Ch-containing fractions were identified by SDS-PAGE, pooled, dialyzed against PBS and concentrated (Centiprep YM-30). The protein concentration was determined by quantitative amino acid analysis.

### Example 5

### D3H44-Fab Variants and Mouse-Human Variable Domain Swap

To assess the contributions of the variable light (D3H44-V_{L}) and variable heavy chain (D3H44-V_{H}) to the epitope, two chimeric Fabs were constructed. Both contain the D3H44-Fab constant regions, but they combine a human and a murine variable domain (huV_{L}muV_{H} and muV_{L}huV_{H}). The production in E. coli and purification of D3 antibody Fabs was carried out as described (Presta, L. et al. (2001), supra). In addition, two variable domain swab Fabs were made. The plasmids were constructed by restriction digestion of the parental plasmids for murine D3 and D3H44 and then ligating together the appropriate pieces encoding the light and heavy chains. The combination of the murine D3 variable light domain (muV_{L}) with the D3H44 variable heavy domain (huV_{H}) plus the D3H44 constant domains yielded the Fab variant muV_{L}huV_{H}. The combination of the murine D3 variable heavy domain (muV_{H}) and the D3H44 variable light domain (huV_{L}) plus the D3H44 constant domains yielded the Fab variant huV_{L}muV_{H}. These domain swap Fabs were expressed in E. coli and purified according to methods previously described (Presta, L. et al. (2001), supra).

### Example 6

### 6A6 monoclonal antibody binds to D3H44 variable region

MaxiSorp 96-well microwell plates (Nunc, Roskilde, Denmark) were coated overnight at 4° C. with 1 ug/ml 6A6 in 50 mM carbonate buffer, pH 9.6. Alternatively, the plates were coated overnight at 4° C. with 1 ug/ml 6A6Ch in 50 mM carbonate buffer, pH 9.6 when antibody 5G6 was tested (data not shown). Plates were blocked with 0.5% bovine serum albumin, 0.001% Proclin 300 (Supelco, Bellefonte, Pa.) in phosphate buffered saline (PBS). Two-fold serially diluted antibodies in PBS (e.g., 5G6, D3Ch, D3H13, the Herceptin® antibody, D3H18, D3H44, huV_{L}muV_{H}, or muV_{L}huV_{H}) containing 0.5% bovine serum albumin and 0.05% polysorbate 20 were added to the plates. After a 2 hour incubation, antibodies that bound to the plates were detected by adding peroxidase conjugated goat anti-human F(ab′)₂ antibody (Jackson ImmunoResearch, West Grove, Pa.) or goat anti-mouse FC (Jackson ImmunoResearch, West Grove, Pa.) as necessary, followed by 3,3′,5,5′-tetramethyl benzidine (Kirkegaard & Perry Laboratories, Gaithersburg, Md.) as the substrate. Plates were washed between steps and all incubations were performed at room temperature on an orbital shaker. Absorbance was read at 450 nm on a kinetic microplate reader (Molecular Devices, Sunnyvale, Calif.). The titration curve was fitted using a four-parameter nonlinear regression curve-fitting program (KaleidaGraph, Synergy software, Reading, Pa.).

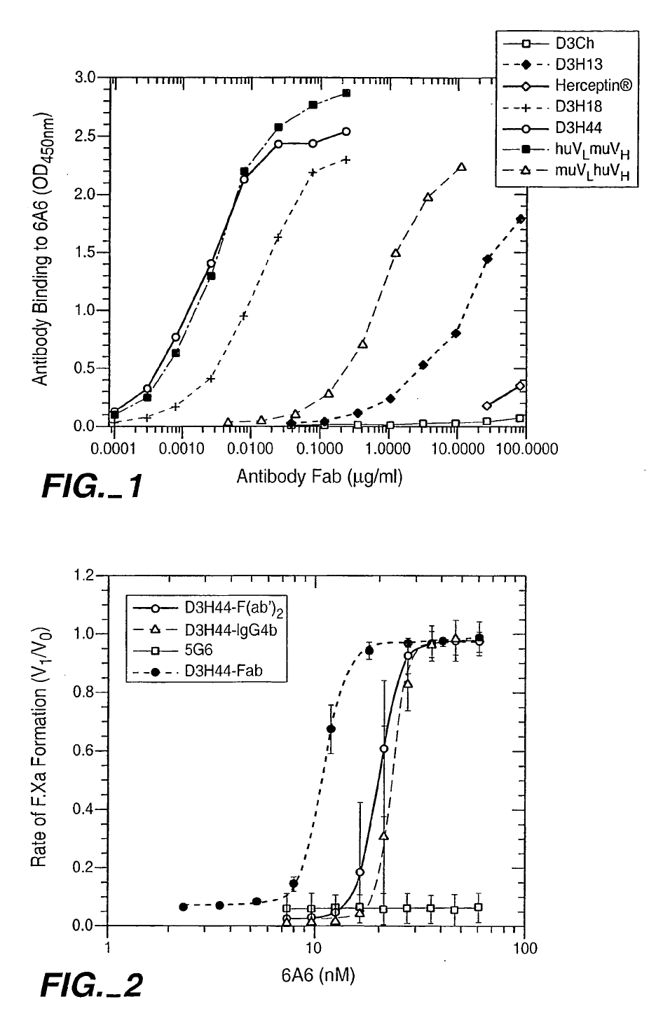

FIG. 1 shows that a 6A6 antibody did not bind to the Herceptin® antibody (an anti-p185^{HER2} antibody) (Carter, P. et al., (1992) supra) or to the chimeric D3Ch antibody (murine D3 antibody variable domain/D3H44 constant domain), both of which have constant regions identical to D3H44 but differ in their variable regions. 6A6 antibody did not bind to another antibody that binds tissue factor in a similar region, 5G6 (Kirchhofer, D. et al., (2000), supra) (data not shown). Together, these observations indicate that 6A6 binds to the variable domains of D3H44-Fab.

To assess the contributions of the variable light (D3H44-VL) and variable heavy (D3H44-VH) domains to the epitope, two chimeric Fabs were constructed. BOth contain the D3H44-Fab constant domains, but they combine a human with a murine variable domain (huV_{L}muV_{H} or muV_{L}huV_{H}). FIG. 1 also shows that 6A6 bound equally well to huV_{L}muV_{H} and D3H44-Fab, but much less well to muV_{L}huV_{H}, suggesting that D3H44-V_{L} makes more important contributions to the idiotope than D3H⁴⁴-V_{H}. D3H18-Fab, which differs from D3H13-Fab only by two amino acid changes in the V_{L} domain (Ser L34→Asn and Leu L46→Thr), bound to 6A6 with a more than 1000-fold higher affinity than D3H13-Fab, suggesting that light chain residues Asn L34 and Thr LA6 are part of the 6A6 epitope.

### Example 7

### 6A6 Neutralizes D3H44 Activity

The ability of 6A6 to neutralize D3H44 was examined in macromolecular substrate activation assays. The assays were performed essentially as described (Presta, L. et al., Thromb. Haemost (2001) ). Briefly, 293 cells were made to express full length TF (TF₁₋₂₆₃), then membrane fractions of the 293 cells (“mTF₁₋₂₆₃”) were prepared. D3H44-antibodies were incubated at room temperature with increasing concentrations of 6A6 or with buffer (control), F.VIIa and mTF₁₋₂₆₃ in HBSA (20 mM Hepes, pH 7.5 containing 150 mM NaCl, 0.5 mg/ml BSA and 5 mM CaCl₂) buffer. After a 25 min incubation period, the reaction-was started by addition of F.X or F.IX. The concentrations of reactants in this mixture were: 10 nM D3H44-antibodies, 50 μg/ml mTF₁₋₂₆₃ (membrane protein concentration), 0.02 nM F.VIIa (2 nM for F.IX assay), 200 nM F.X or 400 nM F.IX. At different time points, reaction aliquots were taken and the concentrations of generated F.Xa or F.IXa were determined using specific chromogenic substrates [Presta, L. et al., (2001) supra]. The inital rates of substrate activation were calculated by linear regression analysis and expressed as fractional activities (vᵢ/vₒ). A similar protocol was used for F.X activation assays with relipidated truncated TF comprising residues 1-243 (relTF₁₋₂₄₃) [Presta, et al., supra]. The reactant concentrations in the mixture were: 1 nM D3H44-antibodies, 0.1 nM relTF₁₋₂₄₃, 0.02 nM F.Vila and 200 nM F.X.

FIG. 2 and Table 4 (below) show that 6A6 restored the activation of F.X and F.IX in a concentration-dependent fashion, independent of whether D3H44-Fab, D3H44-F(ab′)2 or D3H44-IgG4b were used as inhibitors. At saturating concentrations of 6A6, the rates of F.Xa and of F.IXa formation were similar to control experiments in the absence of any D3H44 inhibitor (92-98% of control activity; Table 4). 6A6 by itself had no effect on macromolecular substrate activation (data not shown). The 6A6 concentration that restored 50% of mTF_{1-263/}FVIIa activity (effective concentration, EC₅₀) was similar for both F.X and F.IX (Table 4) and ranged from 10 nM to about 20 nM. These EC50 values were consistent with the expected 2-fold higher concentration of 6A6 required to neutralize the bivalent inhibitors (D3H44-F(ab′)₂ and D3H44-IgG4b) compared to D3H44-Fab. The specificity of 6A6 was exemplified by lack of binding to 5G6 (FIG. 2), an antibody that binds to a similar or identical TF epitope as D3H44 [see Kirchhofer, D., et al., Thromb. Haemost. 84, 1072-1081 (2000)]. Additional F.X activation assays with relipidated TF₁₋₂₄₃ yielded qualitatively similar results to the mTF assay (Table 4), but the lower D3H44 concentrations used gave EC₅₀ values (1.5-3.6 nM 6A6) that were 5-7-fold lower than in the mTF₁₋₂₆₃ assay. This demonstrates that a competitive inhibitor, which agreed with the finding that 6A6 recognized the D3H44-VL (FIG. 1) domain known to participate in TF binding (Faelber, K. et al., (2001), supra). The results also agreed with ELISA-type competition assays, which showed that 6A6 competed with D3H44 for binding to immobilized TF (data not shown).

TABLE 4
6A6 antibody neutralizes inhibition of TF/FVIIa
complex by D3H44 antibody

| | mTF₁₋₂₆₃ | | rel.TF₁₋₂₄₃ |
|---|---|---|---|
| Inhibitory | F.X | F.IX | F.X |
| antibody | ^{a}EC₅₀ (nM) ± SD | ^{a}EC₅₀ (nM) ± SD | ^{a}EC₅₀ (nM) ± SD |
| D3H44-Fab | 10.9 ± 0.6 (98%) | 9.8 ± 0.5 (98%) | 1.5 ± 0.2 (96%) |
| D3H44-F(ab′)₂ | 19.7 ± 2.9 (97%) | 17.1 ± 0.7 (92%) | 3.6 ± 0.6 (98%) |
| D3H44-IgG4b | 23.0 ± 3.1 (98%) | 19.7 ± 0.1 (97%) | 3.5 ± 0.2 (96%) |
| 5G6 | ^{b}N.E. | ^{b}N.E. | ^{b}N.E. |

^{a}EC₅₀, 50% effective concentration (6A6 concentration that restored 50% of TF/FVIIa activity). In parenthesis are the maximal enzyme activities (in percent of control activity in the absence of 6A6) obtained at saturating concentrations of 6A6.
^{b}N.E., no effect

### Example 8

### Restoration of TF-Dependent Blood Coagulation by 6A6 Antibody

The effects of 6A6 were examined in a whole blood clotting system described by Santucci et al. (Santucci, R. A. et al. (2000), supra). Briefly, whole blood was collected on sodium citrate anticoagulant (0.38% final concentration) from three healthy human volunteers who had not taken any medication known to affect coagulation or platelet function for at least two weeks prior to donation. The whole blood (950 μl) was aliquoted into pairs of sterile plastic tubes (kindly provided by CDI, Inc, Bethesda Md.), one of each pair containing 10 μg lyophilized LPS (E. coli 055:B5 Westphal; Difco; Detroit Mich.), the other empty and serving as control for endotoxin exposure. The blood±endotoxin was incubated in a 37° C. waterbath for 3 hours. In separate tubes, anti-tissue-factor antibody D3H44 F(ab′)₂, or D3H44-F(ab), or full-length D3H44 IgG4b, or anti-TF antibody 5G6 were incubated at 37° C. for 10 minutes with either antibody 6A6 or phosphate buffered saline.

Following the incubation periods, the antibody cocktails were added to the endotoxin-stimulated or non-stimulated whole blood aliquots, so that the final concentrations of the anti-TF antibodies in the blood were 10 nM, and the 6A6 concentration was 20-fold greater. The blood was allowed to incubate at 37° C. for an additional 15 minutes, then each sample (300 μl) was transferred to cuvettes, placed in Sonoclot (Sienco, Inc, Wheat Ridge, Colo.) coaguloviscometers and recalcified (120 μM CaCl₂ final concentration in blood). Fibrin formation was measured by increased drag on a vibrating probe inserted into the blood sample. The clotting time was set as the time when the impedence rose 6 units above baseline (custom algorithm for CDI, Inc, provided by Sienco, Inc, Wheat Ridge, Colo.). The effect of the anti-TF antibodies on the endotoxin-mediated decrease in clotting time was compared to that with 6A6 antibody, and to saline controls. Statistical significance was calculated by unpaired 2-tailed t-test using InStat GraphPad software (San Diego, Calif.).

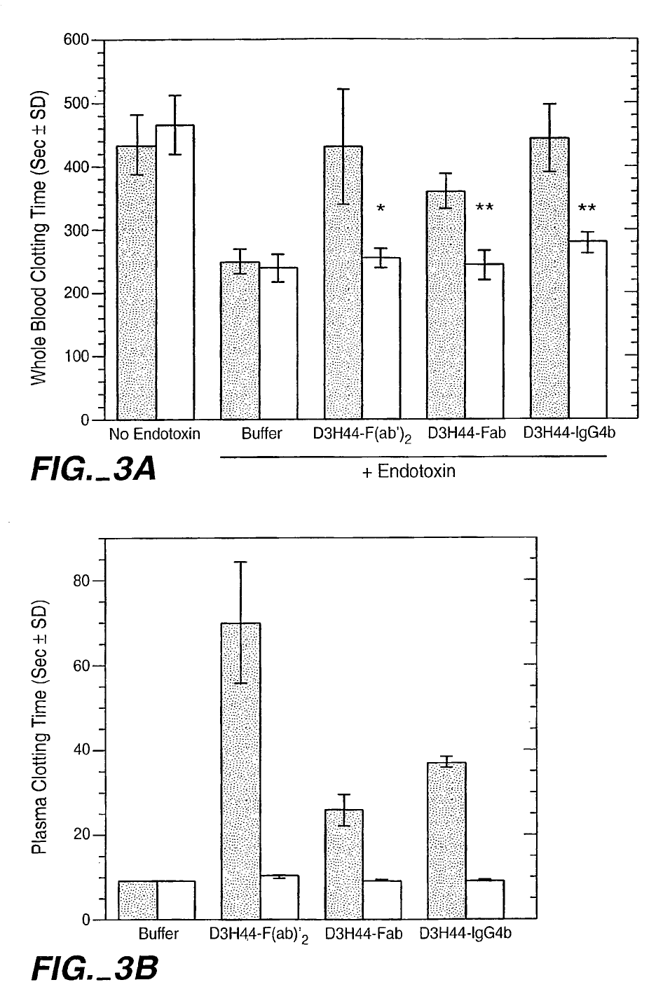

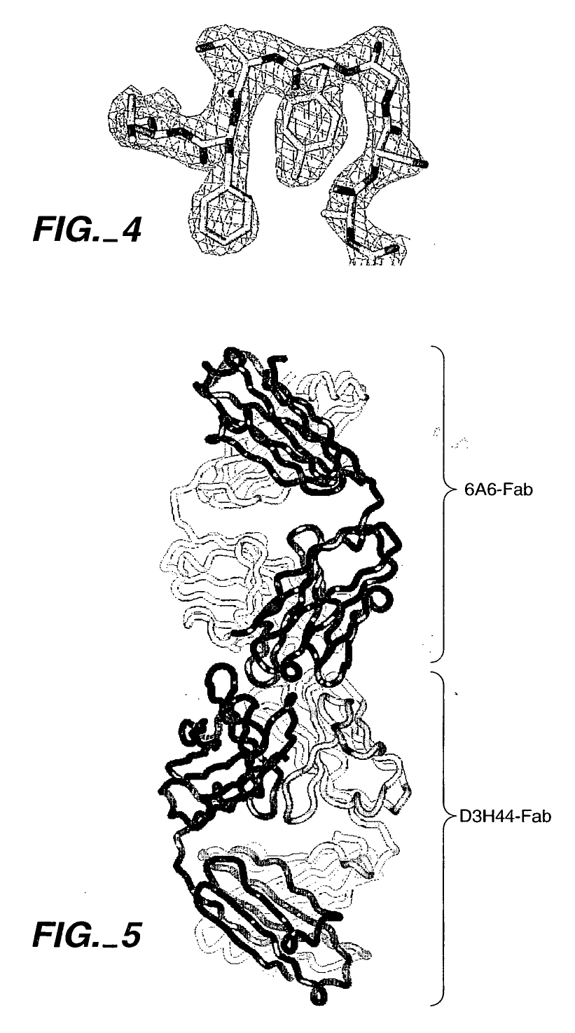

The exposure of citrated human whole blood to endotoxin for 3 hrs shortened the recalcification time by about 50% (FIG. 3a), due to the induction of TF expression on monocytes (Santucci, R. A. et al.(2000), supra). FIG. 3a shows that D3H44 (Fab, F(ab′)₂ or IgG4b) inhibited coagulation resulting in clotting times that were similar to normal recalcification times without endotoxin. FIG. 3a also shows that 6A6 was able to completely neutralize the inhibitory activity of D3H44 resulting in clotting times similar to those of the controls that contained endotoxin only. 6A6 by itself neither affected the normal recalcification time nor the shortened clotting time after endotoxin treatment Moreover, although the D3H44-related antibody 5G6 prolonged clotting time to a similar extent as D3H44, its inhibitory effect was not neutralized by 6A6, confirming the specificity of the 6A6-D3H44 interaction (data not shown).

### Example 9

### Chimeric Mouse/Human 6A6-Fab (6A6Ch-Fab)

A mouse/human chimeric Fab, 6A6Ch-Fab was constructed, expressed and purified as described above (Examples 3 and 4). F.X activation assays with relTF1-243 were performed as described in Example 7 and Presta et-al. (2001), supra. Table 5 (below) shows that the EC50 values of 6A6Ch-Fab were 4.5 nM and 7.4 nM for neutralizing 1 nM of D3H44-Fab and 1 nM of D3H44-F(ab)′₂, respectively, in F.X activation assays with relTF₁₋₂₄₃. As observed with murine full length 6A6 antibody, the EC₅₀ values of 6A6Ch-Fab were about 6- to 7-fold higher in mTF₁₋₂₆₃-based assays (Table 5). In both assay formats saturating concentrations of 6A6Ch-Fab were higher (2 to 3-fold) than for the bivalent 6A6, as expected (compare Tables 4 and 5). Moreover, in PT assays 6A6Ch-Fab at a 13-fold molar excess was able to completely neutralize D3H44-F(ab)′2 (150 nM) and reducing the clotting time from 26.5±0.3 sec (n=3) to the normal value of 10.3±0.3 sec (n=3).

TABLE 5
6A6Ch-Fab chimera neutralizes inhibition
of TF/FVIIa complex by D3H44 antibody

| | mTF₁₋₂₆₃ | rel.TF₁₋₂₄₃ |
|---|---|---|
| | rate of F.Xa formation | rate of F.Xa formation |
| | ^{a}EC₅₀ (nM) ± SD | ^{a}EC₅₀ (nM) ± SD |
| D3H44-Fab | 31.5 ± 0.5 (104.0%) | 4.5 ± 0.6 (96.3%) |
| D3H44-F(ab′)₂ | 42.4 ± 1.2 (95.0%) | 7.4 ± 0.4 (96.5%) |

^{a}EC₅₀, 50% effective concentration (6A6 concentration that restored 50% of TF/FVIIa activity). In parenthesis are the maximal enzyme activities (in percent of control activity in the absence of 6A6) obtained at saturating concentrations of 6A6.

### Example 10

### Neutralization of D3H44 Anticoagulant Activity By 6A6 in Plasma

Prothrombin time (PT) assays were performed by use of an ACL6000 coagulometer (Coulter, Miami, Fla.). Human citrated platelet poor plasma (Stanford Medical School Blood Center, Palo Alto, Calif.) was incubated with 6A6 antibody and D3H44 antibodies for 10 min at room temperature. The plasma concentrations were 1.7 μM for 6A6, 0.3 μM for D3H44-F(ab′)₂ and D3H44-IgG4b, 0.6 μM for D3H44-Fab. Clotting was initated by the addition of human TF (Innovin®, Dade Behring Inc., Newark, Del.) and clotting times were recorded.

FIG. 3b shows that the addition of D3H44 (Fab, F(ab′)₂ or IgG4b) resulted in a 3- to 7-fold prolongation of TF-induced (Innovin®) clotting. Similar to the whole blood clotting assays, the addition of a 3- to 6-fold molar excess of 6A6 (1.7 μM) completely neutralized the anticoagulant activity of D3H44 (FIG. 3b). 6A6 by itself had no effect on the PT. Furthermore, 6A6Ch-Fab at a 13-fold molar excess was able to completely neutralize D3H44-F(ab′)₂ (150 nM) and reduce the clotting time from 26.5±0.3 sec (n=3) to the normal value of 10.2±0.3 sec (n=3) in PT assays.

### Example 11

### Crystallography

Binding and enzymatic assays suggested that 6A6 competes with TF by binding to the antigen combining site of D3H44. Thus, 6A6 fulfilled the criteria of an anti-idiotypic antibody (anti-Id). Some representatives of this antibody class were shown to have a resemblance to the primary antigen, i.e. to carry its ‘internal image’ (Pan, Y. et al., (1995) FASEB J. 9, 4349). The relative affinities of three variants of D3 (D3Ch, D3H13, D3H18) for TF and 6A6 vary in parallel. This could mean that 6A6 recapitulated some of the molecular interactions of TF with D3H44 and, thus, may carry some aspects of an ‘internal image’ of TF. To examine this hypothesis, the structure of the complex of 6A6-Fab with D3H44-Fab was determined using X-ray crystallography.

Fab fragments of the murine 6A6 antibody were produced following standard protocols using papain digestion (Harlow, E. et al., (1988) Cold Spring Harbor Laboratory, 628-629). The 6A6-Fab was purified by Protein A affinity chromatography and used for co-crystallization experiments with D3H44-Fab. A solution containing an excess molar ratio of D3H44-Fab with 6A6-Fab was purified by gel filtration, concentrated to 18 mg/mL in 25 mM Tris-HCl, pH 8.0 and distributed into hanging drops in a sparse matrix of precipitant conditions. Crystals grew from a 1:1 mixture of protein and reservoir containing 20% PEG 3350, 100 nM TRIS, pH 7.0 held at 4° C. Data extending to 2.5Å resolution were collected on a crystal preserved at 100 K in space group P1 at beamline 19-ID of the Structural Biology Center at the Advanced Photon Source and reduced using Denzo/Scalepack (Otwinowski, Z. et al., (1996) Methods in Enzymol 276, 307-326). The cell parameters (a=78.9 Å, b=85.8 Å, c=92.4 Å, α=77.5 Å, β=75.9 Å, γ=63.3 Å) and calculated molecular weight led to a Matthews coefficient Vm=2.91 Å³/Dalton, assuming 2 complexes per asymmetric unit.

The structure was solved by molecular replacement with Amore (Navaza, J. et al., (1994) Acta Crystallogr. A50, 157-163) using Fab search probes optimized by a scan of the rotation function maximum versus elbow angle. Complete placement of two D3H44-Fab molecules (Faelber, K. et al., (2001), supra) led to a correlation coefficient on structure factor amplitudes (cc) of 0.302 (data 12-4 Å). The search probe for the 6A6Ch-Fab (Protein Data Bank (“PDB”) entry 1GIG; http://www.rcsb.org/pdb/) (Bizebard, T., et al., (1991) Acta Crystallogr. B47, 549-555) was chosen from the PDB using a sequence homology criterion (64% identical heavy chain, 92% identical light chain). Placement of two 1GIG Fabs increased the cc to 0.379. Inspection of the complete solution confirmed close contacts between the D3H44-Fab and 1GIG (˜=6A6-Fab) antigen binding regions in both of two highly similar complexes.

Inspection of electron density maps and model modification were performed using XtalView (McRee, D. E., Practical Protein Crystallography, 2 Ed., Academic Press, San Diego, 1999)) and refinement was performed using XPLOR98 (Accekrys, San Diego) utilizing individual restrained isotropic thermal factors, non-crystallographic symmetry (NCS) restraints, and a bulk solvent correction. The final model converged to an R-value of 21.9% and an R-free of 26.7%, and includes 274 water atoms. Data collection and reduction, and refinement parameters appear in Table 6 (below). Because the sequences of the 6A6 V_{L} and V_{H} domains, the 6A6 C_{L} and C_{H1} domains are expected to have high homology with the corresponding domains from 1GIG. However, changes to the starting 1GIG C_{L} and C_{H1} sequences were sometimes made (15 changes and 11 changes, respectively)-in response to persistent and convincing features in electron density maps. The resulting constant region sequences should be regarded as tentative.

TABLE 6
Structure Statistics for 6A6-Fab/D3H44-Fab
Data

| Resolution(Å) | Nmeas¹ | Nref² | Complete³ | I/o | Rmerge⁴ | Rwork⁵ | Rfree⁶ |
|---|---|---|---|---|---|---|---|
| 50.0-5.38 | 31649 | 7279 | 100 | 25 | 0.064 | 0.232 | 0.260 |
| 5.38-4.27 | 31966 | 7276 | 100 | 24 | 0.071 | 0.154 | 0.194 |
| 4.27-3.73 | 31942 | 7241 | 99 | 19 | 0.087 | 0.198 | 0.243 |
| 3.73-3.39 | 32130 | 7255 | 99 | 15 | 0.115 | 0.217 | 0.280 |
| 3.39-3.15 | 32148 | 7240 | 99 | 11 | 0.158 | 0.225 | 0.299 |
| 3.15-2.96 | 31890 | 7185 | 99 | 7.7 | 0.220 | 0.225 | 0.282 |
| 2.96-2.82 | 31585 | 7192 | 99 | 5.7 | 0.297 | 0.246 | 0.289 |
| 2.82-2.69 | 31053 | 7232 | 99 | 4.4 | 0.364 | 0.261 | 0.328 |
| 2.69-2.59 | 29550 | 7191 | 99 | 3.4 | 0.418 | 0.288 | 0.358 |
| 2.59-2.50 | 26775 | 7157 | 98 | 2.5 | 0.477 | 0.341 | 0.411 |
| 50.0-2.50 | 310288 | 72248 | 99 | 12 | 0.129 | 0.219 | 0.267 |

Final Model

| contents of model | | | r.m.s deviations | | | |
|---|---|---|---|---|---|---|
| residues | atoms⁷ | waters | bonds | angles | B-factor | NCS⁸ |
| 1717 | 13321(39) | 274 | 0.009 Å | 1.6° | 4.9 Å² | <0.1 Å |

¹Nmeas is the total number of observations measured.
²Nref is the number of unique reflections measured at least once.
³Complete is the percentage of possible reflections actually measured at least once.
⁴Rmerge = Σ||I| − |<I>||/Σ|<I>|, where I is the intensity of a single observation and <I> the average intensity for symmetry equivalent observations.
⁵Rwork = Σ|Fo − Fc|/Σ|Fo|, where Fo and Fc are observed and calculated structure factor amplitudes, respectively.
⁶Rfree = Rwork for 2193 reflections (3%) sequestered from refinement, selected at random from 99 resolution shells. R for all reflections is 0.220.
⁷Number in parenthesis is number of atoms assigned zero occupancy.
⁸A large majority of residues were restrained to structural conformity in pairs of 100% homologous immunoglobulin domains relating the two complete complexes in the asymmetric unit.

The coordinates of the crystals have been deposited at the Protein Data Bank (Berman, H M et al., (2000) The Protein Data Bank. Nucl. Acid Res. 235-242; http://www.rcsb.org/pdb/) and are presented below in Table 7 (below). The two 6A6-Fab/DeH44-Fab complexes in the crystallographic asymmetric unit are highly similar and will be considered identical. For instance, comparison of the two 6A6-Fab/D3H44-Fab complexes allows 855 Cα pairs. The 6A6 and D3H44 antigen binding regions combine across an interface of 1125 Å² (Broger, C (2000) xsae version 1.5. F. Hoffmann-La Roche, Basel Switzerland; Smith et al., (1985)

A Computer Program For The Calculation of Molecular Volume and Surface Area of Proteins, Merck, Sharpe and Dohme, Research Lab, QCPE; http://qcpe.chem.indiana.edu/cgi-bin/view_catalog.pl?section=10.B).

The two antibody fragments both use their “top” surfaces (i.e., antigen binding regions) in the interaction, with a pseudo-elbow angle relating the 6A6 and D3H44 variable regions of 170 degrees, and related by a rotation of 90 degrees around the long axis with respect to each other (FIG. 5). Calculation of the shape complementarity (Lawrence, et al., (1993) J. Mol. Biol. 234:946-950) between 6A6-Fab and D3H44-Fab yields sc=0.68, which is typical antibody/antigen complexes and which is near the sc=0.71 that was calculated for the TF/D3H44-Fab complex. The 6A6-Fab chain traces are complete for residues 1 to 206 (light chains) and 1 to 213 (heavy chains) (Kabat numbering is used throughout) (Kabat et al., (1991) Sequences of Proteins of Immunological Interest. National Institutes of Health, Bethesda, Md.). The D3H44-Fab light chains are complete for residues 1 to 213, and the D3H44-Fab heavy chains are complete for residues 1 to 213 except for residues 129 to 132, which cannot be traced in weak electron density. The overall average refined temperature factor is 32 Å².

Eighty percent of the main chain torsion angles falls into the most favored region of a Ramachandran diagram (Laskowski, et al., (1993) J. Appl. Cryst. 26:283-291), 10% into the allowed region, 1% (12 residues) in the generously allowed region, and 0.5% (8 residues) in the disallowed region. The 6A6 variable domains are closely homologous with PDB entry 1GIG (Bizebard, T. et al., (1993) Acta Crystallogr. D50, 768-777). The CDRs adopt conformations defining L1₃, L2, L3₁ₐ, H1₁, and H2₂ according to the canonical forms of Al-Lazikani et al (Al-Lazikani, B. et al., (1997) J. Mol. Biol. 273,927-948). The 6A6-Fab elbow angle is 164°. The number of idiotype/anti-idiotype crystal structures (or Ab1/Ab2) reported in the literature is still relatively small (Evans, S. V. et al., (1994) J. Mol. Biol. 241, 691-705; Bentley, G. A., et al., (1990) Nature (London) 348, 254-257; Ban, N. et al., (1994) Proc. Natl. Acad. Sci. USA 91, 1604-1608; Braden, B. C., et al., (1996) J. Mol. Biol. 264, 137-151). The 6A6-Fab/D3H44-Fab complex shares with all others the head-to-head and 90° rotation arrangement of the opposing variable regions. Consistent with the close approach this allows, all 6 CDRs from both variable regions are usually involved in the interaction, even if only via water mediation.

Table 7 (below) shows points of contacts between 6A6-Fab and D3H44-Fab in distances less than 3.6 Å. In this study, two copies of 6A6-Fab/D3H44-Fab complex are present They are almost identical. Both are listed for completeness. Each polypeptide chain has a single letter code. There are eight polypeptide chains in total. The 6A6-Fab light and heavy chains referred to as “W” and “X,” respectively, are complexed with the D3H44-Fab light and heavy chains referred to as “L” and “H,” respectively. The 6A6-Fab light and heavy chains referred to as “Y” and “Z,” respectively, are complexed with the D3H44-Fab light and heavy chains referred to as “M” and “I,” respectively.

The headers “A,” “B,” “C” and “D” refer to the polypeptide chain (i.e., W, X, L, H, Y, Z, M or I) in which the atom resides, the residue number of the polypeptide chain in which the atom resides (Kabat numbering system), the type of amino acid in which the atom resides and name of the atom (e.g., CE1=carbon epsilon 1). “Distance” refers to the distance between the atom 1 and atom 2.

TABLE 7

| ATOM 1 | | | | ATOM 2 | | | | Distance |
|---|---|---|---|---|---|---|---|---|
| A | B | C | D | A | B | C | D | (\acute{Å}) |
| X | 32 | HIS | CE1 | H | 53 | GLU | OE1 | 3.5147 |
| X | 53 | HIS | NE2 | H | 96 | THR | CG2 | 3.4518 |
| X | 94 | ARG | CZ | H | 53 | GLU | OE2 | 3.5422 |
| X | 94 | ARG | NH1 | H | 53 | GLU | OE2 | 3.3256 |
| X | 94 | ARG | NH1 | H | 53 | GLU | OE1 | 3.4103 |
| X | 94 | ARG | NH2 | H | 53 | GLU | OE2 | 2.8707 |
| X | 98 | ARG | CD | H | 95 | ASP | OD2 | 3.5481 |
| X | 98 | ARG | CD | H | 33 | TYR | CD2 | 3.5714 |
| X | 98 | ARG | CD | H | 33 | TYR | CE2 | 3.5931 |
| X | 98 | ARG | NE | H | 95 | ASP | OD2 | 2.6766 |
| X | 98 | ARG | NE | H | 33 | TYR | CD1 | 3.4587 |
| X | 98 | ARG | NE | H | 33 | TYR | CG | 3.4779 |
| X | 98 | ARG | NE | H | 95 | ASP | CG | 3.5098 |
| X | 98 | ARG | CZ | H | 33 | TYR | CD1 | 3.2963 |
| X | 98 | ARG | CZ | H | 33 | TYR | CE1 | 3.3158 |
| X | 98 | ARG | CZ | H | 95 | ASP | OD2 | 3.5473 |
| X | 98 | ARG | CZ | H | 33 | TYR | CE1 | 3.2230 |
| X | 98 | ARG | NH1 | H | 33 | TYR | CZ | 3.5220 |
| X | 98 | ARG | NH2 | H | 95 | ASP | OD1 | 2.9828 |
| X | 98 | ARG | NH2 | H | 33 | TYR | CD1 | 3.4505 |
| X | 98 | ARG | NH2 | H | 95 | ASP | OD2 | 3.5648 |
| X | 98 | ARG | NH2 | H | 98 | ALA | N | 3.5964 |
| X | 99 | TYR | CE1 | H | 52 | ASP | OD2 | 3.5269 |
| X | 99 | TYR | OH | H | 56 | ASN | ND2 | 2.6546 |
| X | 99 | TYR | OH | H | 52 | ASP | OD2 | 2.9094 |
| X | 99 | TYR | OH | H | 56 | ASN | CB | 3.3682 |
| X | 99 | TYR | OH | H | 56 | ASN | CG | 3.4505 |
| X | 99 | TYR | OH | H | 54 | GLN | OE1 | 3.5570 |
| W | 29 | THR | OG1 | L | 27 | ARG | CD | 3.0294 |
| W | 29 | THR | OG1 | L | 27 | ARG | NE | 3.4131 |
| W | 30 | SER | OG | L | 27 | ARG | CB | 3.3626 |
| W | 30 | SER | OG | L | 28 | ASP | N | 3.4246 |
| W | 32 | TYR | CE1 | L | 28 | ASP | O | 3.4487 |
| W | 32 | TYR | CE2 | L | 92 | GLY | O | 3.3388 |
| W | 32 | TYR | CZ | L | 92 | GLY | O | 3.3865 |
| W | 32 | TYR | OH | L | 92 | GLY | O | 2.6830 |
| W | 32 | TYR | OH | L | 92 | GLY | CA | 3.2186 |
| W | 32 | TYR | OH | L | 92 | GLY | C | 3.2871 |
| W | 32 | TYR | OH | L | 28 | ASP | O | 3.5805 |
| W | 32 | TYR | OH | L | 27 | ARG | NH1 | 3.5846 |
| W | 91 | TRP | NE1 | L | 30 | LYS | CG | 3.3893 |
| W | 91 | TRP | NE1 | L | 28 | ASP | OD2 | 3.5377 |
| W | 93 | SER | CA | L | 28 | ASP | OD2 | 3.3660 |
| W | 96 | TRP | CZ2 | L | 30 | LYS | NZ | 3.4990 |
| X | 50 | LEU | CD1 | L | 50 | TYR | OH | 3.3812 |
| X | 52 | HIS | CD2 | L | 50 | TYR | CG | 3.3871 |
| X | 53 | HIS | ND1 | L | 49 | TYR | OH | 2.7392 |
| X | 53 | HIS | ND1 | L | 49 | TYR | CZ | 3.2889 |
| X | 53 | HIS | ND1 | L | 49 | TYR | CE2 | 3.5238 |
| X | 53 | HIS | CE1 | L | 49 | TYR | OH | 3.5379 |
| X | 54 | ASN | CB | L | 49 | TYR | OH | 3.5142 |
| X | 54 | ASN | CG | L | 53 | SER | OG | 3.5433 |
| X | 54 | ASN | OD1 | L | 53 | SER | OG | 2.7345 |
| X | 54 | ASN | OD1 | L | 53 | SER | CB | 3.5578 |
| X | 95 | GLU | CD | L | 30 | LYS | NZ | 3.5194 |
| X | 95 | GLU | OE1 | L | 30 | LYS | NZ | 3.4782 |
| X | 95 | GLU | OE2 | L | 30 | LYS | NZ | 2.7977 |
| X | 97 | PHE | CB | L | 32 | TYR | CZ | 3.2540 |
| X | 97 | PHE | CB | L | 32 | TYR | CE2 | 3.5034 |
| X | 97 | PHE | CB | L | 32 | TYR | OH | 3.5520 |
| X | 97 | PHE | CB | L | 32 | TYR | CE1 | 3.5666 |
| X | 97 | PHE | O | L | 91 | HIS | ND1 | 2.9184 |
| X | 97 | PHE | O | L | 91 | HIS | O | 3.2583 |
| X | 98 | ARG | O | L | 96 | TRP | NE1 | 2.9097 |
| X | 100A | TYR | CE2 | L | 91 | HIS | O | 3.1963 |
| X | 100A | TYR | CE2 | L | 92 | GLY | O | 3.3689 |
| X | 100A | TYR | CE2 | L | 92 | GLY | C | 3.5406 |
| X | 100A | TYR | CZ | L | 92 | GLY | O | 3.3283 |
| X | 100A | TYR | CZ | L | 91 | HIS | O | 3.3889 |
| X | 100A | TYR | CZ | L | 92 | GLY | C | 3.4456 |
| X | 100A | TYR | CZ | L | 93 | GLU | N | 3.5264 |
| X | 100A | TYR | CZ | L | 93 | GLU | CA | 3.5862 |
| X | 100A | TYR | OH | L | 91 | HIS | O | 2.7135 |
| X | 100A | TYR | OH | L | 93 | GLU | N | 2.9285 |
| X | 100A | TYR | OH | L | 93 | GLU | CA | 3.0104 |
| X | 100A | TYR | OH | L | 93 | GLU | C | 3.1628 |
| X | 100A | TYR | OH | L | 92 | GLY | C | 3.3008 |
| X | 100A | TYR | OH | L | 94 | SER | N | 3.4360 |
| Z | 27 | TYR | CB | I | 53 | GLU | OE2 | 3.4311 |
| Z | 32 | HIS | CE1 | I | 53 | GLU | OE2 | 3.3008 |
| Z | 53 | HIS | CE1 | I | 99 | TYR | OH | 3.1186 |
| Z | 53 | HIS | NE2 | I | 96 | THR | CG2 | 3.4202 |
| Z | 53 | HIS | NE2 | I | 99 | TYR | OH | 3.5809 |
| Z | 94 | ARG | CZ | I | 53 | GLU | OE1 | 3.5174 |
| Z | 94 | ARG | CZ | I | 53 | GLU | OE2 | 3.5216 |
| Z | 94 | ARG | NH1 | I | 53 | GLU | OE1 | 3.2759 |
| Z | 94 | ARG | NH2 | I | 53 | GLU | OE2 | 2.5318 |
| Z | 94 | ARG | NH2 | I | 53 | GLU | OE1 | 2.9139 |
| Z | 94 | ARG | NH2 | I | 53 | GLU | CD | 3.0312 |
| Z | 97 | PHE | CE1 | I | 98 | ALA | CB | 3.5834 |
| Z | 98 | ARG | CD | I | 33 | TYR | CD2 | 3.4711 |
| Z | 98 | ARG | CD | I | 95 | ASP | OD2 | 3.5183 |
| Z | 98 | ARG | CD | I | 33 | TYR | CE2 | 3.5449 |
| Z | 98 | ARG | NE | I | 95 | ASP | OD2 | 2.7666 |
| Z | 98 | ARG | NE | I | 33 | TYR | CG | 3.4802 |
| Z | 98 | ARG | NE | I | 95 | ASP | CG | 3.5461 |
| Z | 98 | ARG | NE | I | 33 | TYR | CD2 | 3.5922 |
| Z | 98 | ARG | CZ | I | 33 | TYR | CD1 | 3.4504 |
| Z | 98 | ARG | CZ | I | 33 | TYR | CE1 | 3.4623 |
| Z | 98 | ARG | NH1 | I | 33 | TYR | CE1 | 3.4205 |
| Z | 98 | ARG | NH1 | I | 33 | TYR | CZ | 3.5084 |
| Z | 98 | ARG | NH2 | I | 95 | ASP | OD1 | 3.1670 |
| Z | 98 | ARG | NH2 | I | 33 | TYR | CD1 | 3.5319 |
| Z | 99 | TYR | CE1 | I | 52 | ASP | OD2 | 3.4768 |
| Z | 99 | TYR | CZ | I | 52 | ASP | OD2 | 3.5093 |
| Z | 99 | TYR | OH | I | 52 | ASP | OD2 | 2.6987 |
| Z | 99 | TYR | OH | I | 56 | ASN | ND2 | 3.0892 |
| Z | 99 | TYR | OH | I | 56 | ASN | CB | 3.4860 |
| Y | 29 | THR | OG1 | M | 27 | ARG | NH1 | 3.5840 |
| Y | 29 | THR | CG2 | M | 27 | ARG | CD | 3.2161 |
| Y | 30 | SER | OG | M | 27 | ARG | CB | 3.4329 |
| Y | 30 | SER | OG | M | 28 | ASP | N | 3.5030 |
| Y | 32 | TYR | CE1 | M | 27 | ARG | NH2 | 3.3027 |
| Y | 32 | TYR | CE1 | M | 28 | ASP | O | 3.5858 |
| Y | 32 | TYR | CE2 | M | 92 | GLY | O | 3.4123 |
| Y | 32 | TYR | CZ | M | 27 | ARG | NH2 | 3.1150 |
| Y | 32 | TYR | CZ | M | 92 | GLY | O | 3.4435 |
| Y | 32 | TYR | OH | M | 92 | GLY | O | 2.6743 |
| Y | 32 | TYR | OH | M | 27 | ARG | NH2 | 2.9573 |
| Y | 32 | TYR | OH | M | 92 | GLY | CA | 3.2781 |
| Y | 32 | TYR | OH | M | 92 | GLY | C | 3.3109 |
| Y | 91 | TRP | NE1 | M | 30 | LYS | CG | 3.3586 |
| Y | 93 | SER | CA | M | 28 | ASP | OD2 | 3.3710 |
| Z | 52 | HIS | CD2 | M | 50 | TYR | CG | 3.5147 |
| Z | 53 | HIS | ND1 | M | 49 | TYR | OH | 2.7051 |
| Z | 53 | HIS | ND1 | M | 49 | TYR | CZ | 3.2473 |
| Z | 53 | HIS | ND1 | M | 49 | TYR | CE2 | 3.5726 |
| Z | 53 | HIS | CE1 | M | 49 | TYR | OH | 3.5485 |
| Z | 54 | ASN | CB | M | 49 | TYR | OH | 3.5518 |
| Z | 54 | ASN | CG | M | 49 | TYR | OH | 3.4906 |
| Z | 54 | ASN | ND2 | M | 53 | SER | OG | 2.6568 |
| Z | 54 | ASN | ND2 | M | 49 | TYR | OH | 2.7650 |
| Z | 54 | ASN | ND2 | M | 53 | SER | CB | 3.0369 |
| Z | 54 | ASN | ND2 | M | 49 | TYR | CZ | 3.2681 |
| Z | 54 | ASN | ND2 | M | 49 | TYR | CE2 | 3.5205 |
| Z | 95 | GLU | OE2 | M | 30 | LYS | NZ | 3.1391 |
| Z | 95 | GLU | OE2 | M | 32 | TYR | OH | 3.5774 |
| Z | 97 | PHE | CB | M | 32 | TYR | CZ | 3.4497 |
| Z | 97 | PHE | CB | M | 32 | TYR | CE2 | 3.5767 |
| Z | 97 | PHE | O | M | 91 | HIS | ND1 | 2.6618 |
| Z | 97 | PHE | O | M | 91 | HIS | O | 3.2120 |
| Z | 97 | PHE | O | M | 91 | HIS | CE1 | 3.3502 |
| Z | 98 | ARG | O | M | 96 | TRP | NE1 | 2.8374 |
| Z | 100A | TYR | CE2 | M | 91 | HIS | O | 3.1930 |
| Z | 100A | TYR | CE2 | M | 92 | GLY | O | 3.5458 |
| Z | 100A | TYR | CZ | M | 91 | HIS | O | 3.2724 |
| Z | 100A | TYR | CZ | M | 92 | GLY | O | 3.4174 |
| Z | 100A | TYR | CZ | M | 92 | GLY | C | 3.5417 |
| Z | 100A | TYR | OH | M | 91 | HIS | O | 2.5975 |
| Z | 100A | TYR | OH | M | 93 | GLU | N | 3.1825 |
| Z | 100A | TYR | OH | M | 93 | GLU | CA | 3.2805 |
| Z | 100A | TYR | OH | M | 93 | GLU | C | 3.3850 |
| Z | 100A | TYR | OH | M | 92 | GLY | C | 3.5109 |
| Z | 100A | TYR | OH | M | 91 | HIS | C | 3.5632 |

Comparison between D3H44-Fab in Complex with 6A6-Fab and in Prior Structures of D3H44

Faelber et al. have reported structures of D3H44-Fab alone and in complex with TF (Faelber, K et al., (2000), supra ). D3H44-Fab was found to be highly similar in these two environments, even to the extent of displaying elbow angles different by only 3°. The present structures are mostly unchanged from this earlier work, except for a large change in the elbow angle. The elbow angles of the prior D3H44-Fab structures were 132° and 135°, but the present structures' elbow angles are 175°. As a result, superposition of intact D3H44-Fab from the TF and 6A6 complexes yields a large rmsd of 3.4 Å for 356Cα pairs. However, variable regions superpose with rms deviations of 0.50 Å and 0.77 Å against free- and TF-bound D3H44-Fab, respectively. The most relevant changes in the present structure arise in CDR-H3 and are associated with 6A6 interactions, discussed in the next section. This area also held the most significant differences between the free- and TF-bound D3H44-Fab characterized by Faelber et al. ((Faelber, K. et al., (2001), supra).

(b) Comparison of TF and 6A6 as Binding Partners of D3H44

The hallmark of antibody-antigen interactions is that they are of both high affinity and high specificity. The immune network hypothesis (Jerne, N. K., Ann. Immunol. (Paris) 125C, 373-389 (1974)) suggests that a series of antibodies may arise in vivo in which the primary antibody (Ab 1) binds antigen, a secondary antibody (Ab2) binds the primary antibody, a tertiary antibody (Ab3) binds the secondary antibody, and so on. When considering such an idea, it is useful to acknowledge that the principal differences among the antibodies in such a series will occur within their respective antigen binding regions. Based on this simple model, the hypothesis leads to the result that Ab2 binds Ab1 in a way that mimics the original antigen, and analogous mimicry is a property of other Abs in the series. Based on the biochemical characterization herein, 6A6 clearly blocks the binding of TF to D3H44 and can thereby serve as an effective functional mimic of TF insofar as it binds D3H44.

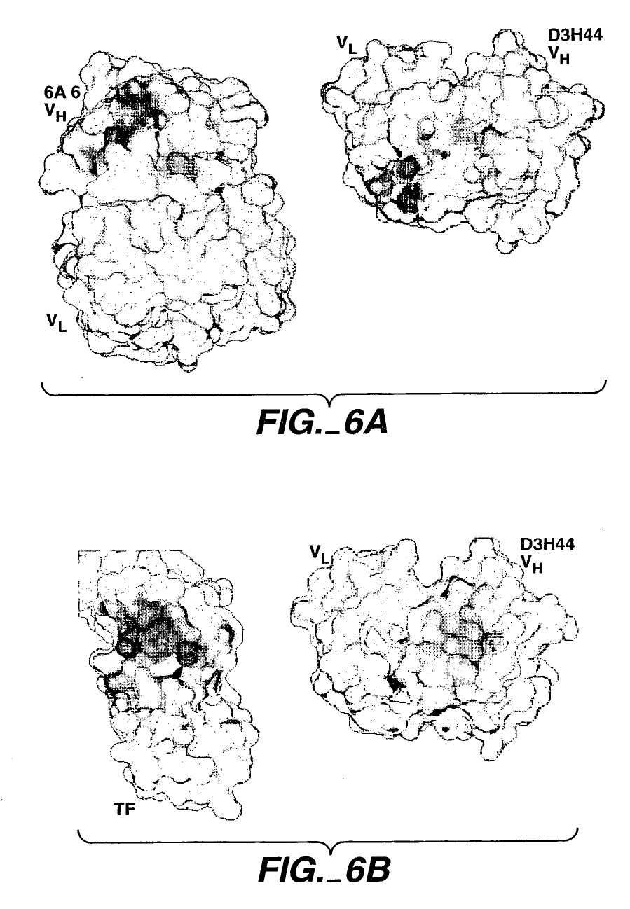

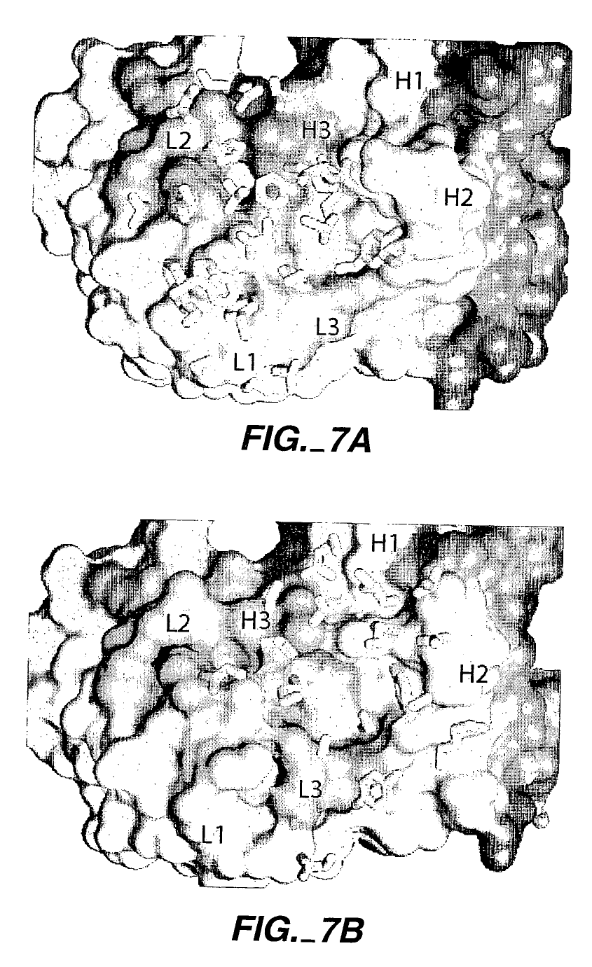

The structures of 6A6 and TF were compared. The degree of structural mimicry between 6A6 and TF is low. As seen above, D3H44-Fab itself is generally quite similar in complex with the two different binding partners TF and 6A6-Fab, and this similarity extends to the distribution of charged side chains in the D3H44-fab combining surface. Both 6A6-Fab and TF offer complimentary arrangements of charged residues to D3H44-Fab (FIG. 6). However, 6A6-Fab contacts significantly more of the D3H44-FabV_{L} and less of the D3H44-FabV_{H} than does TF (FIG. 6). The D3H44-Fab light chain loses about 670 Å² of solvent accessible surface area to 6A6, but only about 280 Å² to TF. Conversely, D3H44-Fab V_{H} loses about 440 Å² to 6A6-Fab, but about 700 Å² to TF. Additionally, the poor correspondence between the types of side chains (acidic, basic, hydrophobic, hydrophilic) presented to D3H44-Fab by the two antigens where their D3H44-Fab contact zones intersect is apparent in FIG. 7.

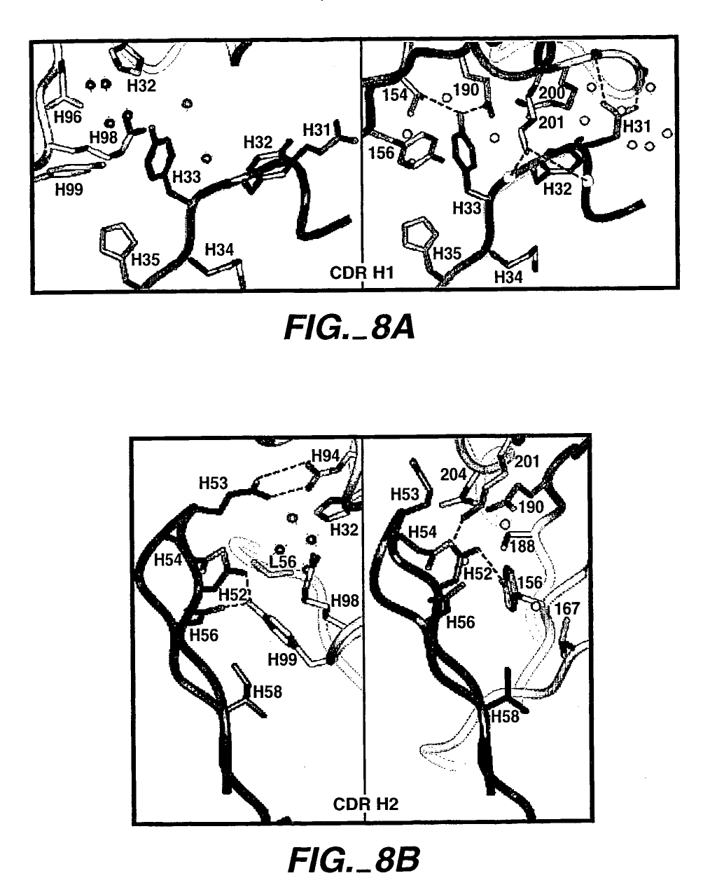

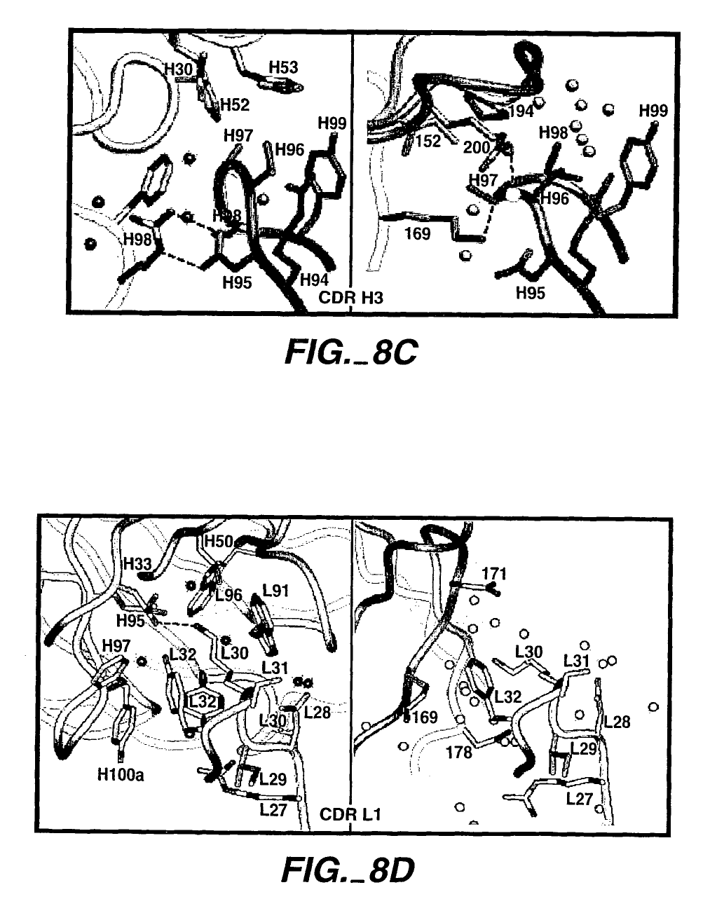

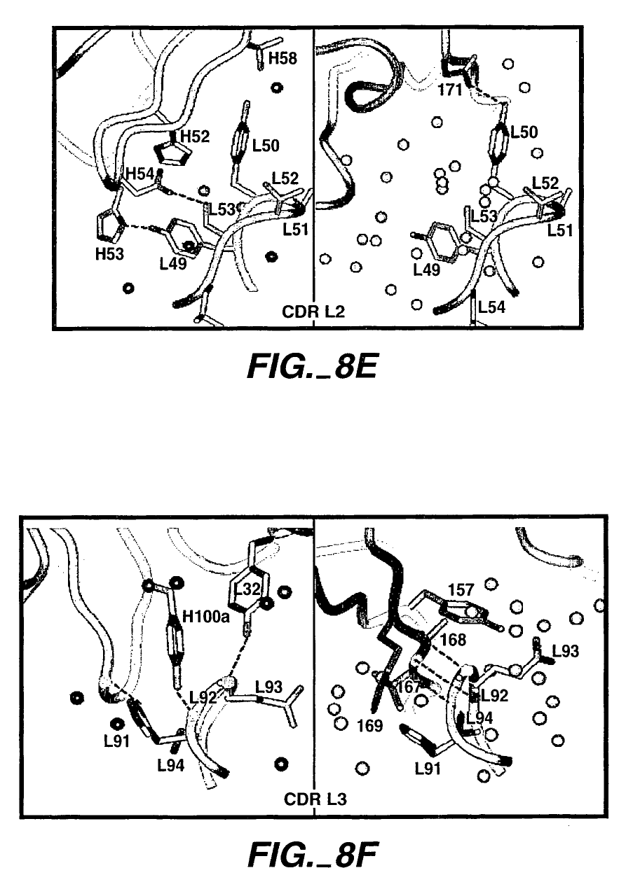

Interactions between D3H44-Fab and both 6A6-Fab and TF are illustrated in FIG. 8, where it is apparent that the D3H44-Fab CDR conformations are very similar in the two complexes, except for CDR-H3 (FIG. 8C). Close correspondence breaks down between some side chain conformations, for instance between residues Glu H53 at the tip of CDR-H2 (FIG. 8b). Such side chain differences combined with subtle changes in the relationships between CDRs to present similar, but not identical, surfaces (FIG. 7). As can be seen in FIGS. 6 and 7, there are clearly a greater number of intimate contacts for CDR-L1 (FIG. 8D) and CDR-L2 (FIG. 8E), and fewer-for CDR-H1 (FIG. 8A), in the 6A6-Fab complex. Despite the rough structural analogy between Arg H102 from 6A6-Fab and Lys169 from TF, the D3H44-Fab CDR-H3 interacts with them in different ways, using the side chain of Asp H99 with the former and the main chain carbonyl oxygen of Ala H101 with the latter (FIG. 8C). At CDR-L3, both 6A6-Fab and TF share three H-bonds with D3H44-Fab, but only one of the atoms from D3H44-Fab is shared in common. There is, however, an isolated structural correspondence between 6A6 and TF where they interact with the D3H44-Fab CDR-H2, as they both present a tyrosine hydroxyl hydrogen atom to the D3H44-Fab Asp52 side chain (FIG. 8B).

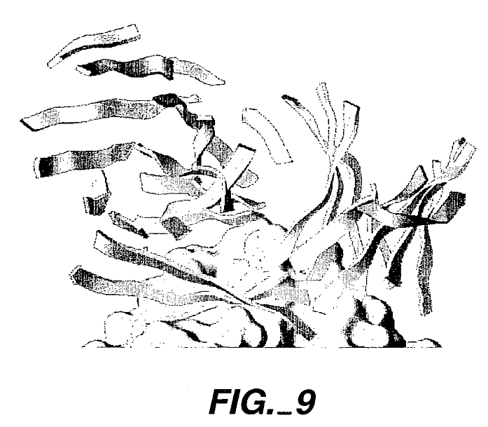

Despite the fact that both TF and 6A6 share a similar overall tertiary structure of β-sandwiches, there is poor correspondence between the ways these elements are presented to D3H44-Fab. The epitope presented by TF is along the edges of its β-sheet(s), but 6A6-Fab presents a binding surface formed from loops connecting the β-strands. Thus, there is a large angle relating the planes of the TF and 6A6 β-sheets, and overall structural mimicry is likely to be poor (FIG. 9). The two proteins are independent binding partners of D3H44 with significant overlap of their binding regions. On this basis, 6A6 is characterized as an anti-idiotypic antibody of type Ab2γ(McRee, (1999), supra).

The substantial overlap of idiotope and antigen combining site also provided the structural explanation why 6A6 effectively reversed the anticoagulant activity of D3H44. In both enzymatic and clotting assays, the restoration of TF/F.VIIa activity by 6A6 was complete, suggesting that 6A6 can be useful as a specific D3H44-directed antidote. Anticoagulants always carry a certain risk of adverse hemorrhagic events and therefore the antidote concept has attracted significant attention. Recent efforts aimed at generating agents that can rapidly and specifically inactivate the anticoagulant component and restore normal hemostasis, as exemplified by the heparin neutralizing agents Neutralase™ (Heres, E. K. et al. Anesth. Analg. 93 (2001)), heparin-binding peptides (Hulin, M. S., et al. J. Vasc. Surg. 26, 1043-1048 (1997); Schick, B. P., et al. Thromb. Haemost. 85, 482-487 (2001)) and recombinant platelet factor 4 (Dehmer, G. J. et al. Circulation 91,-2188-2194 (1995)), and by oligonuclotide based F.IXa inhibitor antidotes (Rusconi, C. P. et al. (2002), supra). Because 6A6 is a full length murine antibody likely to generate an immune response in humans, 6A6 was modified to a mouse/human chimeric antibody Fab fragment (6A6Ch-Fab). This Fab is composed of the murine 6A6 variable and human constant regions, similar to the clinically used anti-GPIIb/IIIa antibody Fab 7E3 (ReoProm™) (Coller, B. S. J. Clin. Invest. 100 (1997)). In-vitro studies demonstrated that 6A6Ch-Fab effectively neutralized the D3H44-F(ab′)² inhibitory activity in plasma clotting assays when present at about a 13-fold molar excess. In vivo, this might translate into relatively low doses of 6A6Ch-Fab required to neutralize D3H44, which is a very potent TF inhibitor and exerts antithrombotic activity at low doses (Presta L. et al. (2001), supra; Bullens, S. et al. (2001), supra).

The 6A6 system includes the additional component of a shift away from the D3H33 V_{H} and towards the D3H44 V_{L}, relative to the TF binding site on D3H44. The relatively large contact with D3H44's V_{L} explains the results with chimeric D3H44 variable regions where 6A6 binding is dependent on the correct version of V_{L} but not of V_{H}. The fact that two changes in the D3H44 V_{L} sequence (Ser L34→Asn and Leu L46 →Thr) have a dramatic effect on 6A6 binding is apparently consistent with this. However, these amino acids do not contact 6A6-Fab, so the effect of their mutation must be indirect. Because they are both in the V_{H}/V_{L} interface, indirect effects may be manifested in one or both variable domains, perhaps via a shift in their overall relationship.

The structural coordinates of the 6A6-Fab and D3H44 antibody are described in Table 8 below and at Protein Data Bank accession number 1PG7.

TABLE 8

| | | | | | | | | | | | | |
|---|---|---|---|---|---|---|---|---|---|---|---|---|
| ATOM | 1 | N | GLU | H | 1 | 11.606 | 27.810 | 9.967 | 1.00 | 49.61 | H | N |
| ATOM | 2 | CA | GLU | H | 1 | 11.179 | 26.420 | 9.830 | 1.00 | 46.81 | H | C |
| ATOM | 3 | CB | GLU | H | 1 | 10.457 | 25.960 | 11.096 | 1.00 | 51.86 | H | C |
| ATOM | 4 | CG | GLU | H | 1 | 9.878 | 24.548 | 11.026 | 1.00 | 54.87 | H | C |
| ATOM | 5 | CD | GLU | H | 1 | 8.561 | 24.395 | 11.803 | 1.00 | 56.65 | H | C |
| ATOM | 6 | OE1 | GLU | H | 1 | 7.817 | 25.399 | 11.941 | 1.00 | 56.76 | H | O |
| ATOM | 7 | OE2 | GLU | H | 1 | 8.264 | 23.265 | 12.260 | 1.00 | 50.62 | H | O |
| ATOM | 8 | C | GLU | H | 1 | 10.242 | 26.291 | 8.641 | 1.00 | 43.95 | H | C |
| ATOM | 9 | O | GLU | H | 1 | 9.159 | 26.870 | 8.618 | 1.00 | 42.10 | H | O |
| ATOM | 10 | N | VAL | H | 2 | 10.675 | 25.524 | 7.653 | 1.00 | 41.43 | H | N |
| ATOM | 11 | CA | VAL | H | 2 | 9.889 | 25.299 | 6.451 | 1.00 | 37.22 | H | C |
| ATOM | 12 | CB | VAL | H | 2 | 10.773 | 24.737 | 5.308 | 1.00 | 37.24 | H | C |
| ATOM | 13 | CG1 | VAL | H | 2 | 9.914 | 24.356 | 4.105 | 1.00 | 38.38 | H | C |
| ATOM | 14 | CG2 | VAL | H | 2 | 11.808 | 25.765 | 4.896 | 1.00 | 34.47 | H | C |
| ATOM | 15 | C | VAL | H | 2 | 8.744 | 24.329 | 6.704 | 1.00 | 34.46 | H | C |
| ATOM | 16 | O | VAL | H | 2 | 8.913 | 23.304 | 7.366 | 1.00 | 33.47 | H | O |
| ATOM | 17 | N | GLN | H | 3 | 7.577 | 24.662 | 6.170 | 1.00 | 32.98 | H | N |
| ATOM | 18 | CA | GLN | H | 3 | 6.414 | 23.799 | 6.311 | 1.00 | 35.58 | H | C |
| ATOM | 19 | CB | GLN | H | 3 | 5.628 | 24.158 | 7.573 | 1.00 | 34.45 | H | C |
| ATOM | 20 | CG | GLN | H | 3 | 4.795 | 23.012 | 8.127 | 1.00 | 44.91 | H | C |
| ATOM | 21 | CD | GLN | H | 3 | 4.260 | 23.314 | 9.513 | 1.00 | 50.81 | H | C |
| ATOM | 22 | OE1 | GLN | H | 3 | 3.069 | 23.599 | 9.695 | 1.00 | 51.35 | H | O |
| ATOM | 23 | NE2 | GLN | H | 3 | 5.144 | 23.264 | 10.502 | 1.00 | 47.43 | H | N |
| ATOM | 24 | C | GLN | H | 3 | 5.524 | 23.870 | 5.061 | 1.00 | 32.04 | H | C |
| ATOM | 25 | O | GLN | H | 3 | 5.421 | 24.912 | 4.404 | 1.00 | 32.99 | H | O |
| ATOM | 26 | N | LEU | H | 4 | 4.940 | 22.730 | 4.706 | 1.00 | 27.35 | H | N |
| ATOM | 27 | CA | LEU | H | 4 | 4.059 | 22.630 | 3.548 | 1.00 | 23.54 | H | C |
| ATOM | 28 | CB | LEU | H | 4 | 4.750 | 21.861 | 2.400 | 1.00 | 16.89 | H | C |
| ATOM | 29 | CG | LEU | H | 4 | 5.881 | 22.567 | 1.632 | 1.00 | 18.84 | H | C |
| ATOM | 30 | CD1 | LEU | H | 4 | 6.596 | 21.618 | 0.657 | 1.00 | 16.50 | H | C |
| ATOM | 31 | CD2 | LEU | H | 4 | 5.321 | 23.780 | 0.871 | 1.00 | 21.43 | H | C |
| ATOM | 32 | C | LEU | H | 4 | 2.807 | 21.897 | 3.995 | 1.00 | 21.70 | H | C |
| ATOM | 33 | O | LEU | H | 4 | 2.887 | 20.773 | 4.495 | 1.00 | 20.33 | H | O |
| ATOM | 34 | N | VAL | H | 5 | 1.658 | 22.558 | 3.876 | 1.00 | 23.34 | H | N |
| ATOM | 35 | CA | VAL | H | 5 | 0.388 | 21.956 | 4.265 | 1.00 | 24.37 | H | C |
| ATOM | 36 | CB | VAL | H | 5 | −0.339 | 22.748 | 5.378 | 1.00 | 27.89 | H | C |
| ATOM | 37 | CG1 | VAL | H | 5 | −1.678 | 22.071 | 5.693 | 1.00 | 24.79 | H | C |
| ATOM | 38 | CG2 | VAL | H | 5 | 0.519 | 22.839 | 6.636 | 1.00 | 23.59 | H | C |
| ATOM | 39 | C | VAL | H | 5 | −0.551 | 21.924 | 3.073 | 1.00 | 32.73 | H | C |
| ATOM | 40 | O | VAL | H | 5 | −0.891 | 22.967 | 2.514 | 1.00 | 36.44 | H | O |
| ATOM | 41 | N | GLU | H | 6 | −0.988 | 20.728 | 2.702 | 1.00 | 29.43 | H | N |
| ATOM | 42 | CA | GLU | H | 6 | −1.904 | 20.593 | 1.589 | 1.00 | 27.91 | H | C |
| ATOM | 43 | CB | GLU | H | 6 | −1.443 | 19.493 | 0.655 | 1.00 | 34.02 | H | C |
| ATOM | 44 | CG | GLU | H | 6 | 0.027 | 19.472 | 0.460 | 1.00 | 39.79 | H | C |
| ATOM | 45 | CD | GLU | H | 6 | 0.517 | 18.073 | 0.286 | 1.00 | 46.66 | H | C |
| ATOM | 46 | OE1 | GLU | H | 6 | −0.008 | 17.374 | −0.600 | 1.00 | 56.86 | H | O |
| ATOM | 47 | OE2 | GLU | H | 6 | 1.404 | 17.656 | 1.043 | 1.00 | 43.67 | H | O |
| ATOM | 48 | C | GLU | H | 6 | −3.337 | 20.305 | 2.030 | 1.00 | 26.07 | H | C |
| ATOM | 49 | O | GLU | H | 6 | −3.597 | 19.740 | 3.096 | 1.00 | 23.68 | H | O |
| ATOM | 50 | N | SER | H | 7 | −4.270 | 20.732 | 1.197 | 1.00 | 20.70 | H | N |
| ATOM | 51 | CA | SER | H | 7 | −5.675 | 20.517 | 1.454 | 1.00 | 28.21 | H | C |
| ATOM | 52 | CB | SER | H | 7 | −6.251 | 21.675 | 2.272 | 1.00 | 29.28 | H | C |
| ATOM | 53 | OG | SER | H | 7 | −5.996 | 22.909 | 1.628 | 1.00 | 33.25 | H | O |
| ATOM | 54 | C | SER | H | 7 | −6.351 | 20.426 | 0.105 | 1.00 | 25.22 | H | C |
| ATOM | 55 | O | SER | H | 7 | −5.748 | 20.725 | −0.918 | 1.00 | 20.28 | H | O |
| ATOM | 56 | N | GLY | H | 8 | −7.609 | 20.021 | 0.111 | 1.00 | 30.34 | H | N |
| ATOM | 57 | CA | GLY | H | 8 | −8.352 | 19.896 | −1.125 | 1.00 | 34.53 | H | C |
| ATOM | 58 | C | GLY | H | 8 | −8.644 | 18.451 | −1.466 | 1.00 | 34.42 | H | C |
| ATOM | 59 | O | GLY | H | 8 | −9.408 | 18.179 | −2.388 | 1.00 | 45.05 | H | O |
| ATOM | 60 | N | GLY | H | 9 | −8.014 | 17.524 | −0.751 | 1.00 | 33.46 | H | N |
| ATOM | 61 | CA | GLY | H | 9 | −8.243 | 16.116 | −1.008 | 1.00 | 28.55 | H | C |
| ATOM | 62 | C | GLY | H | 9 | −9.662 | 15.713 | −0.645 | 1.00 | 35.87 | H | C |
| ATOM | 63 | O | GLY | H | 9 | −10.342 | 16.424 | 0.095 | 1.00 | 35.57 | H | O |
| ATOM | 64 | N | GLY | H | 10 | −10.105 | 14.580 | −1.179 | 1.00 | 33.92 | H | N |
| ATOM | 65 | CA | GLY | H | 10 | −11.441 | 14.090 | −0.911 | 1.00 | 34.01 | H | C |
| ATOM | 66 | C | GLY | H | 10 | −12.010 | 13.249 | −2.040 | 1.00 | 36.19 | H | C |
| ATOM | 67 | O | GLY | H | 10 | −11.343 | 12.986 | −3.037 | 1.00 | 43.14 | H | O |
| ATOM | 68 | N | LEU | H | 11 | −13.263 | 12.842 | −1.876 | 1.00 | 37.33 | H | N |
| ATOM | 69 | CA | LEU | H | 11 | −13.993 | 12.020 | −2.846 | 1.00 | 34.94 | H | C |
| ATOM | 70 | CB | LEU | H | 11 | −15.234 | 11.436 | −2.145 | 1.00 | 36.05 | H | C |
| ATOM | 71 | CG | LEU | H | 11 | −16.213 | 10.469 | −2.819 | 1.00 | 39.03 | H | C |
| ATOM | 72 | CD1 | LEU | H | 11 | −15.466 | 9.275 | −3.417 | 1.00 | 41.52 | H | C |
| ATOM | 73 | CD2 | LEU | H | 11 | −17.250 | 9.989 | −1.801 | 1.00 | 38.02 | H | C |
| ATOM | 74 | C | LEU | H | 11 | −14.399 | 12.782 | −4.126 | 1.00 | 33.35 | H | C |
| ATOM | 75 | O | LEU | H | 11 | −14.988 | 13.868 | −4.056 | 1.00 | 32.80 | H | O |
| ATOM | 76 | N | VAL | H | 12 | −14.058 | 12.226 | −5.289 | 1.00 | 26.97 | H | N |
| ATOM | 77 | CA | VAL | H | 12 | −14.405 | 12.847 | −6.574 | 1.00 | 29.19 | H | C |
| ATOM | 78 | CB | VAL | H | 12 | −13.189 | 13.557 | −7.241 | 1.00 | 31.15 | H | C |
| ATOM | 79 | CG1 | VAL | H | 12 | −13.668 | 14.400 | −8.402 | 1.00 | 35.06 | H | C |
| ATOM | 80 | CG2 | VAL | H | 12 | −12.447 | 14.438 | −6.257 | 1.00 | 33.42 | H | C |
| ATOM | 81 | C | VAL | H | 12 | −14.955 | 11.812 | −7.575 | 1.00 | 29.98 | H | C |
| ATOM | 82 | O | VAL | H | 12 | −14.433 | 10.689 | −7.689 | 1.00 | 27.88 | H | O |
| ATOM | 83 | N | GLN | H | 13 | −16.004 | 12.197 | −8.302 | 1.00 | 25.95 | H | N |
| ATOM | 84 | CA | GLN | H | 13 | −16.624 | 11.322 | −9.301 | 1.00 | 26.18 | H | C |
| ATOM | 85 | CB | GLN | H | 13 | −17.943 | 11.913 | −9.785 | 1.00 | 28.45 | H | C |
| ATOM | 86 | CG | GLN | H | 13 | −18.861 | 12.448 | −8.714 | 1.00 | 38.23 | H | C |
| ATOM | 87 | CD | GLN | H | 13 | −19.699 | 11.383 | −8.055 | 1.00 | 36.25 | H | C |
| ATOM | 88 | OE1 | GLN | H | 13 | −20.706 | 11.691 | −7.427 | 1.00 | 49.21 | H | O |
| ATOM | 89 | NE2 | GLN | H | 13 | −19.310 | 10.125 | −8.207 | 1.00 | 39.35 | H | N |
| ATOM | 90 | C | GLN | H | 13 | −15.726 | 11.207 | −10.537 | 1.00 | 31.62 | H | C |
| ATOM | 91 | O | GLN | H | 13 | −15.029 | 12.163 | −10.901 | 1.00 | 30.61 | H | O |
| ATOM | 92 | N | PRO | H | 14 | −15.736 | 10.042 | −11.213 | 1.00 | 24.57 | H | N |
| ATOM | 93 | CD | PRO | H | 14 | −16.374 | 8.761 | −10.880 | 1.00 | 26.39 | H | C |
| ATOM | 94 | CA | PRO | H | 14 | −14.902 | 9.902 | −12.409 | 1.00 | 22.79 | H | C |
| ATOM | 95 | CB | PRO | H | 14 | −15.211 | 8.488 | −12.872 | 1.00 | 17.75 | H | C |
| ATOM | 96 | CG | PRO | H | 14 | −15.472 | 7.781 | −11.596 | 1.00 | 24.49 | H | C |
| ATOM | 97 | C | PRO | H | 14 | −15.348 | 10.937 | −13.444 | 1.00 | 22.05 | H | C |
| ATOM | 98 | O | PRO | H | 14 | −16.545 | 11.148 | −13.666 | 1.00 | 25.13 | H | O |
| ATOM | 99 | N | GLY | H | 15 | −14.381 | 11.646 | −14.006 | 1.00 | 26.46 | H | N |
| ATOM | 100 | CA | GLY | H | 15 | −14.698 | 12.666 | −14.981 | 1.00 | 20.98 | H | C |
| ATOM | 101 | C | GLY | H | 15 | −14.679 | 14.028 | −14.323 | 1.00 | 28.48 | H | C |
| ATOM | 102 | O | GLY | H | 15 | −14.642 | 15.054 | −15.016 | 1.00 | 23.38 | H | O |
| ATOM | 103 | N | GLY | H | 16 | −14.704 | 14.036 | −12.986 | 1.00 | 29.42 | H | N |
| ATOM | 104 | CA | GLY | H | 16 | −14.679 | 15.285 | −12.237 | 1.00 | 28.94 | H | C |
| ATOM | 105 | C | GLY | H | 16 | −13.299 | 15.928 | −12.190 | 1.00 | 35.39 | H | C |
| ATOM | 106 | O | GLY | H | 16 | −12.376 | 15.516 | −12.904 | 1.00 | 33.25 | H | O |
| ATOM | 107 | N | SER | H | 17 | −13.148 | 16.928 | −11.325 | 1.00 | 40.44 | H | N |
| ATOM | 108 | CA | SER | H | 17 | −11.881 | 17.639 | −11.175 | 1.00 | 43.15 | H | C |
| ATOM | 109 | CB | SER | H | 17 | −11.998 | 19.062 | −11.722 | 1.00 | 38.77 | H | C |
| ATOM | 110 | OG | SER | H | 17 | −12.411 | 19.072 | −13.071 | 1.00 | 51.60 | H | O |
| ATOM | 111 | C | SER | H | 17 | −11.444 | 17.735 | −9.720 | 1.00 | 46.51 | H | C |
| ATOM | 112 | O | SER | H | 17 | −12.266 | 17.675 | −8.807 | 1.00 | 50.48 | H | O |
| ATOM | 113 | N | LEU | H | 18 | −10.140 | 17.895 | −9.517 | 1.00 | 48.42 | H | N |
| ATOM | 114 | CA | LEU | H | 18 | −9.583 | 18.055 | −8.182 | 1.00 | 47.09 | H | C |
| ATOM | 115 | CB | LEU | H | 18 | −8.712 | 16.845 | −7.782 | 1.00 | 47.40 | H | C |
| ATOM | 116 | CG | LEU | H | 18 | −7.500 | 16.364 | −8.601 | 1.00 | 48.99 | H | C |
| ATOM | 117 | CD1 | LEU | H | 18 | −6.287 | 17.276 | −8.463 | 1.00 | 44.44 | H | C |
| ATOM | 118 | CD2 | LEU | H | 18 | −7.125 | 14.982 | −8.126 | 1.00 | 50.30 | H | C |
| ATOM | 119 | C | LEU | H | 18 | −8.793 | 19.365 | −8.132 | 1.00 | 44.72 | H | C |
| ATOM | 120 | O | LEU | H | 18 | −8.150 | 19.757 | −9.108 | 1.00 | 44.47 | H | O |
| ATOM | 121 | N | ARG | H | 19 | −8.898 | 20.066 | −7.012 | 1.00 | 42.36 | H | N |
| ATOM | 122 | CA | ARG | H | 19 | −8.194 | 21.328 | −6.833 | 1.00 | 43.25 | H | C |
| ATOM | 123 | CB | ARG | H | 19 | −9.196 | 22.491 | −6.840 | 1.00 | 46.67 | H | C |
| ATOM | 124 | CG | ARG | H | 19 | −8.638 | 23.819 | −7.372 | 1.00 | 52.85 | H | C |
| ATOM | 125 | CD | ARG | H | 19 | −7.760 | 24.557 | −6.365 | 1.00 | 53.92 | H | C |
| ATOM | 126 | NE | ARG | H | 19 | −7.219 | 25.793 | −6.931 | 1.00 | 56.44 | H | N |
| ATOM | 127 | CZ | ARG | H | 19 | −7.543 | 27.019 | −6.524 | 1.00 | 58.68 | H | C |
| ATOM | 128 | NH1 | ARG | H | 19 | −8.417 | 27.191 | −5.538 | 1.00 | 58.58 | H | N |
| ATOM | 129 | NH2 | ARG | H | 19 | −6.967 | 28.076 | −7.085 | 1.00 | 54.16 | H | N |
| ATOM | 130 | C | ARG | H | 19 | −7.481 | 21.240 | −5.486 | 1.00 | 39.82 | H | C |
| ATOM | 131 | O | ARG | H | 19 | −8.107 | 21.366 | −4.439 | 1.00 | 37.19 | H | O |
| ATOM | 132 | N | LEU | H | 20 | −6.186 | 20.951 | −5.520 | 1.00 | 32.65 | H | N |
| ATOM | 133 | CA | LEU | H | 20 | −5.410 | 20.840 | −4.297 | 1.00 | 24.82 | H | C |
| ATOM | 134 | CB | LEU | H | 20 | −4.477 | 19.628 | −4.377 | 1.00 | 17.90 | H | C |
| ATOM | 135 | CG | LEU | H | 20 | −5.088 | 18.316 | −4.879 | 1.00 | 17.29 | H | C |
| ATOM | 136 | CD1 | LEU | H | 20 | −4.057 | 17.220 | −4.922 | 1.00 | 20.29 | H | C |
| ATOM | 137 | CD2 | LEU | H | 20 | −6.203 | 17.897 | −3.993 | 1.00 | 25.45 | H | C |
| ATOM | 138 | C | LEU | H | 20 | −4.610 | 22.122 | −4.056 | 1.00 | 22.55 | H | C |
| ATOM | 139 | O | LEU | H | 20 | −4.223 | 22.806 | −4.994 | 1.00 | 28.56 | H | O |
| ATOM | 140 | N | SER | H | 21 | −4.398 | 22.470 | −2.796 | 1.00 | 26.18 | H | N |
| ATOM | 141 | CA | SER | H | 21 | −3.620 | 23.657 | −2.473 | 1.00 | 30.60 | H | C |
| ATOM | 142 | CB | SER | H | 21 | −4.503 | 24.758 | −1.862 | 1.00 | 30.85 | H | C |
| ATOM | 143 | OG | SER | H | 21 | −5.100 | 24.321 | −0.666 | 1.00 | 39.93 | H | O |
| ATOM | 144 | C | SER | H | 21 | −2.476 | 23.273 | −1.540 | 1.00 | 30.19 | H | C |
| ATOM | 145 | O | SER | H | 21 | −2.543 | 22.273 | −0.826 | 1.00 | 33.54 | H | O |
| ATOM | 146 | N | CYS | H | 22 | −1.405 | 24.048 | −1.585 | 1.00 | 30.04 | H | N |
| ATOM | 147 | CA | CYS | H | 22 | −0.241 | 23.785 | −0.755 | 1.00 | 31.84 | H | C |
| ATOM | 148 | C | CYS | H | 22 | 0.219 | 25.082 | −0.102 | 1.00 | 29.04 | H | C |
| ATOM | 149 | O | CYS | H | 22 | 0.853 | 25.926 | −0.741 | 1.00 | 33.45 | H | O |
| ATOM | 150 | CB | CYS | H | 22 | 0.866 | 23.219 | −1.620 | 1.00 | 33.70 | H | C |
| ATOM | 151 | SG | CYS | H | 22 | 2.335 | 22.687 | −0.706 | 1.00 | 47.29 | H | S |
| ATOM | 152 | N | ALA | H | 23 | −0.148 | 25.257 | 1.162 | 1.00 | 29.78 | H | N |
| ATOM | 153 | CA | ALA | H | 23 | 0.199 | 26.464 | 1.908 | 1.00 | 30.83 | H | C |
| ATOM | 154 | CB | ALA | H | 23 | −0.814 | 26.697 | 3.053 | 1.00 | 31.10 | H | C |
| ATOM | 155 | C | ALA | H | 23 | 1.611 | 26.347 | 2.451 | 1.00 | 25.06 | H | C |
| ATOM | 156 | O | ALA | H | 23 | 1.915 | 25.423 | 3.199 | 1.00 | 31.50 | H | O |
| ATOM | 157 | N | ALA | H | 24 | 2.486 | 27.239 | 2.001 | 1.00 | 22.33 | H | N |
| ATOM | 158 | CA | ALA | H | 24 | 3.873 | 27.245 | 2.439 | 1.00 | 21.70 | H | C |
| ATOM | 159 | CB | ALA | H | 24 | 4.796 | 27.578 | 1.265 | 1.00 | 19.47 | H | C |
| ATOM | 160 | C | ALA | H | 24 | 4.113 | 28.241 | 3.573 | 1.00 | 25.09 | H | C |
| ATOM | 161 | O | ALA | H | 24 | 3.393 | 29.235 | 3.715 | 1.00 | 24.97 | H | O |
| ATOM | 162 | N | SER | H | 25 | 5.115 | 27.947 | 4.394 | 1.00 | 25.40 | H | N |
| ATOM | 163 | CA | SER | H | 25 | 5.514 | 28.847 | 5.465 | 1.00 | 29.60 | H | C |
| ATOM | 164 | CB | SER | H | 25 | 4.673 | 28.663 | 6.738 | 1.00 | 29.69 | H | C |
| ATOM | 165 | OG | SER | H | 25 | 4.861 | 27.389 | 7.318 | 1.00 | 38.14 | H | O |
| ATOM | 166 | C | SER | H | 25 | 6.991 | 28.619 | 5.730 | 1.00 | 30.42 | H | C |
| ATOM | 167 | O | SER | H | 25 | 7.509 | 27.513 | 5.512 | 1.00 | 28.35 | H | O |
| ATOM | 168 | N | GLY | H | 26 | 7.675 | 29.693 | 6.119 | 1.00 | 30.50 | H | N |
| ATOM | 169 | CA | GLY | H | 26 | 9.091 | 29.615 | 6.422 | 1.00 | 27.68 | H | C |
| ATOM | 170 | C | GLY | H | 26 | 9.990 | 29.883 | 5.233 | 1.00 | 32.77 | H | C |
| ATOM | 171 | O | GLY | H | 26 | 11.214 | 29.738 | 5.323 | 1.00 | 37.91 | H | O |
| ATOM | 172 | N | PHE | H | 27 | 9.383 | 30.269 | 4.118 | 1.00 | 26.14 | H | N |
| ATOM | 173 | CA | PHE | H | 27 | 10.105 | 30.567 | 2.891 | 1.00 | 26.89 | H | C |
| ATOM | 174 | CB | PHE | H | 27 | 10.786 | 29.302 | 2.338 | 1.00 | 33.92 | H | C |
| ATOM | 175 | CG | PHE | H | 27 | 9.833 | 28.292 | 1.735 | 1.00 | 33.27 | H | C |
| ATOM | 176 | CD1 | PHE | H | 27 | 9.084 | 27.442 | 2.545 | 1.00 | 32.52 | H | C |
| ATOM | 177 | CD2 | PHE | H | 27 | 9.696 | 28.188 | 0.356 | 1.00 | 30.62 | H | C |
| ATOM | 178 | CE1 | PHE | H | 27 | 8.219 | 26.505 | 1.986 | 1.00 | 32.77 | H | C |
| ATOM | 179 | CE2 | PHE | H | 27 | 8.833 | 27.255 | −0.208 | 1.00 | 30.18 | H | C |
| ATOM | 180 | CZ | PHE | H | 27 | 8.095 | 26.413 | 0.607 | 1.00 | 31.13 | H | C |
| ATOM | 181 | C | PHE | H | 27 | 9.089 | 31.120 | 1.887 | 1.00 | 31.72 | H | C |
| ATOM | 182 | O | PHE | H | 27 | 7.880 | 31.114 | 2.153 | 1.00 | 23.62 | H | O |
| ATOM | 183 | N | ASN | H | 28 | 9.574 | 31.625 | 0.755 | 1.00 | 31.15 | H | N |
| ATOM | 184 | CA | ASN | H | 28 | 8.688 | 32.175 | −0.260 | 1.00 | 32.05 | H | C |
| ATOM | 185 | CB | ASN | H | 28 | 9.144 | 33.569 | −0.670 | 1.00 | 35.27 | H | C |
| ATOM | 186 | CG | ASN | H | 28 | 9.297 | 34.487 | 0.490 | 1.00 | 37.57 | H | C |
| ATOM | 187 | OD1 | ASN | H | 28 | 10.415 | 34.885 | 0.837 | 1.00 | 42.23 | H | O |
| ATOM | 188 | ND2 | ASN | H | 28 | 8.177 | 34.840 | 1.112 | 1.00 | 39.17 | H | N |
| ATOM | 189 | C | ASN | H | 28 | 8.722 | 31.288 | −1.483 | 1.00 | 36.18 | H | C |
| ATOM | 190 | O | ASN | H | 28 | 9.781 | 31.136 | −2.106 | 1.00 | 36.73 | H | O |
| ATOM | 191 | N | ILE | H | 29 | 7.571 | 30.725 | −1.849 | 1.00 | 39.29 | H | N |
| ATOM | 192 | CA | ILE | H | 29 | 7.501 | 29.854 | −3.026 | 1.00 | 38.99 | H | C |
| ATOM | 193 | CB | ILE | H | 29 | 6.078 | 29.291 | −3.275 | 1.00 | 42.16 | H | C |
| ATOM | 194 | CG2 | ILE | H | 29 | 5.661 | 28.343 | −2.157 | 1.00 | 43.19 | H | C |
| ATOM | 195 | CG1 | ILE | H | 29 | 5.076 | 30.424 | −3.446 | 1.00 | 44.42 | H | C |
| ATOM | 196 | CD1 | ILE | H | 29 | 3.708 | 29.932 | −3.814 | 1.00 | 50.32 | H | C |
| ATOM | 197 | C | ILE | H | 29 | 7.966 | 30.590 | −4.279 | 1.00 | 35.83 | H | C |
| ATOM | 198 | O | ILE | H | 29 | 8.472 | 29.977 | −5.212 | 1.00 | 39.95 | H | O |
| ATOM | 199 | N | LYS | H | 30 | 7.824 | 31.910 | −4.261 | 1.00 | 33.16 | H | N |
| ATOM | 200 | CA | LYS | H | 30 | 8.212 | 32.784 | −5.359 | 1.00 | 39.19 | H | C |
| ATOM | 201 | CB | LYS | H | 30 | 8.033 | 34.242 | −4.902 | 1.00 | 45.04 | H | C |
| ATOM | 202 | CG | LYS | H | 30 | 8.445 | 35.371 | −5.865 | 1.00 | 47.55 | H | C |
| ATOM | 203 | CD | LYS | H | 30 | 8.076 | 36.720 | −5.221 | 1.00 | 51.76 | H | C |
| ATOM | 204 | CE | LYS | H | 30 | 8.781 | 37.921 | −5.847 | 1.00 | 56.77 | H | C |
| ATOM | 205 | NZ | LYS | H | 30 | 8.159 | 38.397 | −7.108 | 1.00 | 60.70 | H | N |
| ATOM | 206 | C | LYS | H | 30 | 9.654 | 32.531 | −5.801 | 1.00 | 41.79 | H | C |
| ATOM | 207 | O | LYS | H | 30 | 9.967 | 32.620 | −6.989 | 1.00 | 37.41 | H | O |
| ATOM | 208 | N | GLU | H | 31 | 10.518 | 32.179 | −4.850 | 1.00 | 40.38 | H | N |
| ATOM | 209 | CA | GLU | H | 31 | 11.915 | 31.939 | −5.166 | 1.00 | 38.28 | H | C |
| ATOM | 210 | CB | GLU | H | 31 | 12.832 | 32.818 | −4.296 | 1.00 | 44.91 | H | C |
| ATOM | 211 | CG | GLU | H | 31 | 12.769 | 32.596 | −2.795 | 1.00 | 52.43 | H | C |
| ATOM | 212 | CD | GLU | H | 31 | 13.505 | 33.691 | −2.012 | 1.00 | 59.06 | H | C |
| ATOM | 213 | OE1 | GLU | H | 31 | 12.880 | 34.746 | −1.745 | 1.00 | 60.97 | H | O |
| ATOM | 214 | OE2 | GLU | H | 31 | 14.700 | 33.499 | −1.666 | 1.00 | 57.74 | H | O |
| ATOM | 215 | C | GLU | H | 31 | 12.391 | 30.492 | −5.188 | 1.00 | 36.70 | H | C |
| ATOM | 216 | O | GLU | H | 31 | 13.593 | 30.246 | −5.177 | 1.00 | 38.90 | H | O |
| ATOM | 217 | N | TYR | H | 32 | 11.465 | 29.537 | −5.274 | 1.00 | 31.50 | H | N |
| ATOM | 218 | CA | TYR | H | 32 | 11.839 | 28.120 | −5.314 | 1.00 | 27.53 | H | C |
| ATOM | 219 | CB | TYR | H | 32 | 11.589 | 27.460 | −3.952 | 1.00 | 27.01 | H | C |
| ATOM | 220 | CG | TYR | H | 32 | 12.662 | 27.646 | −2.895 | 1.00 | 33.63 | H | C |
| ATOM | 221 | CD1 | TYR | H | 32 | 12.668 | 28.773 | −2.058 | 1.00 | 29.31 | H | C |
| ATOM | 222 | CE1 | TYR | H | 32 | 13.625 | 28.919 | −1.061 | 1.00 | 27.44 | H | C |
| ATOM | 223 | CD2 | TYR | H | 32 | 13.648 | 26.671 | −2.696 | 1.00 | 29.30 | H | C |
| ATOM | 224 | CE2 | TYR | H | 32 | 14.609 | 26.812 | −1.698 | 1.00 | 27.60 | H | C |
| ATOM | 225 | CZ | TYR | H | 32 | 14.593 | 27.939 | −0.884 | 1.00 | 30.40 | H | C |
| ATOM | 226 | OH | TYR | H | 32 | 15.549 | 28.084 | 0.102 | 1.00 | 31.46 | H | O |
| ATOM | 227 | C | TYR | H | 32 | 11.061 | 27.316 | −6.359 | 1.00 | 27.29 | H | C |
| ATOM | 228 | O | TYR | H | 32 | 9.937 | 27.662 | −6.716 | 1.00 | 25.89 | H | O |
| ATOM | 229 | N | TYR | H | 33 | 11.663 | 26.234 | −6.839 | 1.00 | 24.13 | H | N |
| ATOM | 230 | CA | TYR | H | 33 | 10.980 | 25.349 | −7.772 | 1.00 | 25.93 | H | C |
| ATOM | 231 | CB | TYR | H | 33 | 11.969 | 24.424 | −8.487 | 1.00 | 29.85 | H | C |
| ATOM | 232 | CG | TYR | H | 33 | 12.736 | 25.066 | −9.604 | 1.00 | 37.28 | H | C |
| ATOM | 233 | CD1 | TYR | H | 33 | 13.939 | 25.727 | −9.359 | 1.00 | 37.59 | H | C |
| ATOM | 234 | CE1 | TYR | H | 33 | 14.628 | 26.355 | −10.387 | 1.00 | 39.94 | H | C |
| ATOM | 235 | CD2 | TYR | H | 33 | 12.248 | 25.038 | −10.908 | 1.00 | 38.14 | H | C |
| ATOM | 236 | CE2 | TYR | H | 33 | 12.928 | 25.654 | −11.934 | 1.00 | 39.93 | H | C |
| ATOM | 237 | CZ | TYR | H | 33 | 14.116 | 26.319 | −11.671 | 1.00 | 42.88 | H | C |
| ATOM | 238 | OH | TYR | H | 33 | 14.772 | 26.991 | −12.684 | 1.00 | 44.05 | H | O |
| ATOM | 239 | C | TYR | H | 33 | 10.065 | 24.481 | −6.906 | 1.00 | 27.14 | H | C |
| ATOM | 240 | O | TYR | H | 33 | 10.541 | 23.832 | −5.965 | 1.00 | 24.91 | H | O |
| ATOM | 241 | N | MET | H | 34 | 8.767 | 24.475 | −7.226 | 1.00 | 27.94 | H | N |
| ATOM | 242 | CA | MET | H | 34 | 7.760 | 23.678 | −6.508 | 1.00 | 29.71 | H | C |
| ATOM | 243 | CB | MET | H | 34 | 6.576 | 24.558 | −6.085 | 1.00 | 29.76 | H | C |
| ATOM | 244 | CG | MET | H | 34 | 6.897 | 25.694 | −5.125 | 1.00 | 27.92 | H | C |
| ATOM | 245 | SD | MET | H | 34 | 7.571 | 25.113 | −3.564 | 1.00 | 35.47 | H | S |
| ATOM | 246 | CE | MET | H | 34 | 6.101 | 24.512 | −2.748 | 1.00 | 17.54 | H | C |
| ATOM | 247 | C | MET | H | 34 | 7.236 | 22.551 | −7.422 | 1.00 | 32.62 | H | C |
| ATOM | 248 | O | MET | H | 34 | 6.925 | 22.786 | −8.596 | 1.00 | 29.75 | H | O |
| ATOM | 249 | N | HIS | H | 35 | 7.140 | 21.335 | −6.887 | 1.00 | 26.97 | H | N |
| ATOM | 250 | CA | HIS | H | 35 | 6.659 | 20.189 | −7.659 | 1.00 | 20.25 | H | C |
| ATOM | 251 | CB | HIS | H | 35 | 7.732 | 19.100 | −7.752 | 1.00 | 23.96 | H | C |
| ATOM | 252 | CG | HIS | H | 35 | 9.031 | 19.533 | −8.353 | 1.00 | 24.70 | H | C |
| ATOM | 253 | CD2 | HIS | H | 35 | 9.791 | 20.634 | −8.156 | 1.00 | 29.43 | H | C |
| ATOM | 254 | ND1 | HIS | H | 35 | 9.743 | 18.726 | −9.213 | 1.00 | 32.47 | H | N |
| ATOM | 255 | CE1 | HIS | H | 35 | 10.889 | 19.308 | −9.515 | 1.00 | 30.72 | H | C |
| ATOM | 256 | NE2 | HIS | H | 35 | 10.944 | 20.466 | −8.886 | 1.00 | 35.05 | H | N |
| ATOM | 257 | C | HIS | H | 35 | 5.444 | 19.519 | −7.002 | 1.00 | 29.82 | H | C |
| ATOM | 258 | O | HIS | H | 35 | 5.092 | 19.792 | −5.844 | 1.00 | 30.96 | H | O |
| ATOM | 259 | N | TRP | H | 36 | 4.820 | 18.618 | −7.754 | 1.00 | 27.11 | H | N |
| ATOM | 260 | CA | TRP | H | 36 | 3.703 | 17.830 | −7.256 | 1.00 | 22.46 | H | C |
| ATOM | 261 | CB | TRP | H | 36 | 2.389 | 18.231 | −7.928 | 1.00 | 19.62 | H | C |
| ATOM | 262 | CG | TRP | H | 36 | 1.783 | 19.483 | −7.342 | 1.00 | 16.78 | H | C |
| ATOM | 263 | CD2 | TRP | H | 36 | 0.951 | 19.573 | −6.181 | 1.00 | 13.61 | H | C |
| ATOM | 264 | CE2 | TRP | H | 36 | 0.595 | 20.937 | −6.023 | 1.00 | 13.15 | H | C |
| ATOM | 265 | CE3 | TRP | H | 36 | 0.459 | 18.635 | −5.267 | 1.00 | 12.58 | H | C |
| ATOM | 266 | CD1 | TRP | H | 36 | 1.902 | 20.753 | −7.822 | 1.00 | 21.40 | H | C |
| ATOM | 267 | NE1 | TRP | H | 36 | 1.188 | 21.635 | −7.041 | 1.00 | 19.32 | H | N |
| ATOM | 268 | CZ2 | TRP | H | 36 | −0.224 | 21.390 | −4.982 | 1.00 | 14.62 | H | C |
| ATOM | 269 | CZ3 | TRP | H | 36 | −0.363 | 19.084 | −4.223 | 1.00 | 17.76 | H | C |
| ATOM | 270 | CH2 | TRP | H | 36 | −0.696 | 20.453 | −4.094 | 1.00 | 16.07 | H | C |
| ATOM | 271 | C | TRP | H | 36 | 4.074 | 16.381 | −7.569 | 1.00 | 24.85 | H | C |
| ATOM | 272 | O | TRP | H | 36 | 4.511 | 16.074 | −8.681 | 1.00 | 22.73 | H | O |
| ATOM | 273 | N | VAL | H | 37 | 3.994 | 15.526 | −6.549 | 1.00 | 23.69 | H | N |
| ATOM | 274 | CA | VAL | H | 37 | 4.315 | 14.111 | −6.669 | 1.00 | 23.60 | H | C |
| ATOM | 275 | CB | VAL | H | 37 | 5.579 | 13.797 | −5.844 | 1.00 | 23.23 | H | C |
| ATOM | 276 | CG1 | VAL | H | 37 | 5.856 | 12.305 | −5.812 | 1.00 | 16.38 | H | C |
| ATOM | 277 | CG2 | VAL | H | 37 | 6.786 | 14.544 | −6.438 | 1.00 | 19.63 | H | C |
| ATOM | 278 | C | VAL | H | 37 | 3.122 | 13.335 | −6.116 | 1.00 | 25.66 | H | C |
| ATOM | 279 | O | VAL | H | 37 | 2.419 | 13.852 | −5.270 | 1.00 | 30.18 | H | O |
| ATOM | 280 | N | ARG | H | 38 | 2.874 | 12.118 | −6.593 | 1.00 | 22.55 | H | N |
| ATOM | 281 | CA | ARG | H | 38 | 1.737 | 11.348 | −6.084 | 1.00 | 23.72 | H | C |
| ATOM | 282 | CB | ARG | H | 38 | 0.480 | 11.533 | −6.965 | 1.00 | 24.90 | H | C |
| ATOM | 283 | CG | ARG | H | 38 | 0.548 | 10.734 | −8.252 | 1.00 | 30.15 | H | C |
| ATOM | 284 | CD | ARG | H | 38 | −0.601 | 10.976 | −9.168 | 1.00 | 26.42 | H | C |
| ATOM | 285 | NE | ARG | H | 38 | −0.514 | 10.108 | −10.340 | 1.00 | 28.11 | H | N |
| ATOM | 286 | CZ | ARG | H | 38 | −1.450 | 10.033 | −11.284 | 1.00 | 31.88 | H | C |
| ATOM | 287 | NH1 | ARG | H | 38 | −2.545 | 10.779 | −11.196 | 1.00 | 27.00 | H | N |
| ATOM | 288 | NH2 | ARG | H | 38 | −1.300 | 9.204 | −12.308 | 1.00 | 19.82 | H | N |
| ATOM | 289 | C | ARG | H | 38 | 2.063 | 9.865 | −5.948 | 1.00 | 22.32 | H | C |
| ATOM | 290 | O | ARG | H | 38 | 3.027 | 9.359 | −6.522 | 1.00 | 25.72 | H | O |
| ATOM | 291 | N | GLN | H | 39 | 1.221 | 9.171 | −5.203 | 1.00 | 17.06 | H | N |
| ATOM | 292 | CA | GLN | H | 39 | 1.426 | 7.775 | −4.968 | 1.00 | 21.19 | H | C |
| ATOM | 293 | CB | GLN | H | 39 | 2.281 | 7.612 | −3.713 | 1.00 | 18.17 | H | C |
| ATOM | 294 | CG | GLN | H | 39 | 2.805 | 6.213 | −3.488 | 1.00 | 20.79 | H | C |
| ATOM | 295 | CD | GLN | H | 39 | 3.787 | 6.148 | −2.326 | 1.00 | 20.76 | H | C |
| ATOM | 296 | OE1 | GLN | H | 39 | 3.778 | 7.008 | −1.450 | 1.00 | 19.15 | H | O |
| ATOM | 297 | NE2 | GLN | H | 39 | 4.623 | 5.111 | −2.306 | 1.00 | 13.09 | H | N |
| ATOM | 298 | C | GLN | H | 39 | 0.083 | 7.087 | −4.788 | 1.00 | 22.69 | H | C |
| ATOM | 299 | O | GLN | H | 39 | −0.642 | 7.369 | −3.844 | 1.00 | 20.89 | H | O |
| ATOM | 300 | N | ALA | H | 40 | −0.262 | 6.221 | −5.733 | 1.00 | 24.19 | H | N |
| ATOM | 301 | CA | ALA | H | 40 | −1.509 | 5.461 | −5.665 | 1.00 | 28.99 | H | C |
| ATOM | 302 | CB | ALA | H | 40 | −1.812 | 4.802 | −7.014 | 1.00 | 17.63 | H | C |
| ATOM | 303 | C | ALA | H | 40 | −1.354 | 4.399 | −4.581 | 1.00 | 28.97 | H | C |
| ATOM | 304 | O | ALA | H | 40 | −0.242 | 3.961 | −4.283 | 1.00 | 30.11 | H | O |
| ATOM | 305 | N | PRO | H | 41 | −2.470 | 3.964 | −3.985 | 1.00 | 32.96 | H | N |
| ATOM | 306 | CD | PRO | H | 41 | −3.847 | 4.375 | −4.317 | 1.00 | 34.33 | H | C |
| ATOM | 307 | CA | PRO | H | 41 | −2.468 | 2.946 | −2.928 | 1.00 | 33.97 | H | C |
| ATOM | 308 | CB | PRO | H | 41 | −3.945 | 2.568 | −2.828 | 1.00 | 33.27 | H | C |
| ATOM | 309 | CG | PRO | H | 41 | −4.631 | 3.866 | −3.128 | 1.00 | 31.77 | H | C |
| ATOM | 310 | C | PRO | H | 41 | −1.607 | 1.728 | −3.262 | 1.00 | 33.23 | H | C |
| ATOM | 311 | O | PRO | H | 41 | −1.811 | 1.079 | −4.296 | 1.00 | 36.61 | H | O |
| ATOM | 312 | N | GLY | H | 42 | −0.643 | 1.439 | −2.385 | 1.00 | 30.53 | H | N |
| ATOM | 313 | CA | GLY | H | 42 | 0.260 | 0.309 | −2.573 | 1.00 | 26.98 | H | C |
| ATOM | 314 | C | GLY | H | 42 | 1.188 | 0.346 | −3.786 | 1.00 | 30.00 | H | C |
| ATOM | 315 | O | GLY | H | 42 | 1.775 | −0.677 | −4.142 | 1.00 | 30.75 | H | O |
| ATOM | 316 | N | LYS | H | 43 | 1.345 | 1.508 | −4.416 | 1.00 | 25.95 | H | N |
| ATOM | 317 | CA | LYS | H | 43 | 2.196 | 1.598 | −5.596 | 1.00 | 31.72 | H | C |
| ATOM | 318 | CB | LYS | H | 43 | 1.360 | 2.035 | −6.806 | 1.00 | 35.81 | H | C |
| ATOM | 319 | CG | LYS | H | 43 | 0.208 | 1.056 | −7.103 | 1.00 | 41.81 | H | C |
| ATOM | 320 | CD | LYS | H | 43 | −0.383 | 1.218 | −8.502 | 1.00 | 51.42 | H | C |
| ATOM | 321 | CE | LYS | H | 43 | −1.596 | 0.280 | −8.735 | 1.00 | 59.39 | H | C |
| ATOM | 322 | NZ | LYS | H | 43 | −2.823 | 0.631 | −7.926 | 1.00 | 60.24 | H | N |
| ATOM | 323 | C | LYS | H | 43 | 3.416 | 2.490 | −5.400 | 1.00 | 31.30 | H | C |
| ATOM | 324 | O | LYS | H | 43 | 3.657 | 2.975 | −4.294 | 1.00 | 34.34 | H | O |
| ATOM | 325 | N | GLY | H | 44 | 4.206 | 2.659 | −6.459 | 1.00 | 30.88 | H | N |
| ATOM | 326 | CA | GLY | H | 44 | 5.398 | 3.489 | −6.393 | 1.00 | 21.93 | H | C |
| ATOM | 327 | C | GLY | H | 44 | 5.087 | 4.966 | −6.527 | 1.00 | 26.72 | H | C |
| ATOM | 328 | O | GLY | H | 44 | 3.914 | 5.345 | −6.596 | 1.00 | 24.90 | H | O |
| ATOM | 329 | N | LEU | H | 45 | 6.137 | 5.796 | −6.561 | 1.00 | 22.12 | H | N |
| ATOM | 330 | CA | LEU | H | 45 | 6.005 | 7.253 | −6.673 | 1.00 | 19.30 | H | C |
| ATOM | 331 | CB | LEU | H | 45 | 7.199 | 7.933 | −5.997 | 1.00 | 18.98 | H | C |
| ATOM | 332 | CG | LEU | H | 45 | 7.263 | 7.709 | −4.501 | 1.00 | 19.41 | H | C |
| ATOM | 333 | CD1 | LEU | H | 45 | 8.630 | 8.099 | −3.972 | 1.00 | 18.33 | H | C |
| ATOM | 334 | CD2 | LEU | H | 45 | 6.157 | 8.528 | −3.864 | 1.00 | 12.34 | H | C |
| ATOM | 335 | C | LEU | H | 45 | 5.946 | 7.688 | −8.132 | 1.00 | 19.70 | H | C |
| ATOM | 336 | O | LEU | H | 45 | 6.556 | 7.044 | −8.981 | 1.00 | 15.40 | H | O |
| ATOM | 337 | N | GLU | H | 46 | 5.230 | 8.781 | −8.406 | 1.00 | 19.98 | H | N |
| ATOM | 338 | CA | GLU | H | 46 | 5.078 | 9.330 | −9.760 | 1.00 | 24.09 | H | C |
| ATOM | 339 | CB | GLU | H | 46 | 3.661 | 9.126 | −10.316 | 1.00 | 30.26 | H | C |
| ATOM | 340 | CG | GLU | H | 46 | 3.073 | 7.719 | −10.388 | 1.00 | 41.56 | H | C |
| ATOM | 341 | CD | GLU | H | 46 | 1.646 | 7.722 | −10.983 | 1.00 | 42.39 | H | C |
| ATOM | 342 | OE1 | GLU | H | 46 | 1.442 | 8.337 | −12.060 | 1.00 | 38.70 | H | O |
| ATOM | 343 | OE2 | GLU | H | 46 | 0.726 | 7.139 | −10.359 | 1.00 | 43.61 | H | O |
| ATOM | 344 | C | GLU | H | 46 | 5.254 | 10.839 | −9.705 | 1.00 | 25.71 | H | C |
| ATOM | 345 | O | GLU | H | 46 | 4.705 | 11.500 | −8.817 | 1.00 | 29.69 | H | O |
| ATOM | 346 | N | TRP | H | 47 | 5.941 | 11.387 | −10.702 | 1.00 | 23.36 | H | N |
| ATOM | 347 | CA | TRP | H | 47 | 6.157 | 12.828 | −10.800 | 1.00 | 19.28 | H | C |
| ATOM | 348 | CB | TRP | H | 47 | 7.515 | 13.097 | −11.460 | 1.00 | 22.36 | H | C |
| ATOM | 349 | CG | TRP | H | 47 | 7.908 | 14.556 | −11.544 | 1.00 | 25.90 | H | C |
| ATOM | 350 | CD2 | TRP | H | 47 | 7.898 | 15.380 | −12.717 | 1.00 | 27.35 | H | C |
| ATOM | 351 | CE2 | TRP | H | 47 | 8.315 | 16.665 | −12.324 | 1.00 | 20.54 | H | C |
| ATOM | 352 | CE3 | TRP | H | 47 | 7.567 | 15.154 | −14.061 | 1.00 | 28.50 | H | C |
| ATOM | 353 | CD1 | TRP | H | 47 | 8.321 | 15.353 | −10.524 | 1.00 | 21.72 | H | C |
| ATOM | 354 | NE1 | TRP | H | 47 | 8.566 | 16.619 | −10.982 | 1.00 | 18.83 | H | N |
| ATOM | 355 | CZ2 | TRP | H | 47 | 8.415 | 17.731 | −13.223 | 1.00 | 29.94 | H | C |
| ATOM | 356 | CZ3 | TRP | H | 47 | 7.663 | 16.214 | −14.960 | 1.00 | 27.86 | H | C |
| ATOM | 357 | CH2 | TRP | H | 47 | 8.083 | 17.488 | −14.534 | 1.00 | 32.13 | H | C |
| ATOM | 358 | C | TRP | H | 47 | 5.021 | 13.395 | −11.661 | 1.00 | 22.06 | H | C |
| ATOM | 359 | O | TRP | H | 47 | 4.692 | 12.831 | −12.699 | 1.00 | 20.40 | H | O |
| ATOM | 360 | N | VAL | H | 48 | 4.382 | 14.464 | −11.191 | 1.00 | 23.82 | H | N |
| ATOM | 361 | CA | VAL | H | 48 | 3.295 | 15.095 | −11.933 | 1.00 | 20.20 | H | C |
| ATOM | 362 | CB | VAL | H | 48 | 2.183 | 15.644 | −11.003 | 1.00 | 20.68 | H | C |
| ATOM | 363 | CG1 | VAL | H | 48 | 1.051 | 16.286 | −11.832 | 1.00 | 18.72 | H | C |
| ATOM | 364 | CG2 | VAL | H | 48 | 1.617 | 14.552 | −10.173 | 1.00 | 11.45 | H | C |
| ATOM | 365 | C | VAL | H | 48 | 3.846 | 16.263 | −12.742 | 1.00 | 30.77 | H | C |
| ATOM | 366 | O | VAL | H | 48 | 3.613 | 16.354 | −13.960 | 1.00 | 29.53 | H | O |
| ATOM | 367 | N | GLY | H | 49 | 4.544 | 17.166 | −12.050 | 1.00 | 25.61 | H | N |
| ATOM | 368 | CA | GLY | H | 49 | 5.131 | 18.332 | −12.697 | 1.00 | 35.99 | H | C |
| ATOM | 369 | C | GLY | H | 49 | 5.766 | 19.361 | −11.761 | 1.00 | 35.83 | H | C |
| ATOM | 370 | O | GLY | H | 49 | 5.817 | 19.164 | −10.546 | 1.00 | 42.22 | H | O |
| ATOM | 371 | N | LEU | H | 50 | 6.241 | 20.465 | −12.332 | 1.00 | 32.64 | H | N |
| ATOM | 372 | CA | LEU | H | 50 | 6.861 | 21.529 | −11.550 | 1.00 | 30.79 | H | C |
| ATOM | 373 | CB | LEU | H | 50 | 8.405 | 21.370 | −11.548 | 1.00 | 27.15 | H | C |
| ATOM | 374 | CG | LEU | H | 50 | 9.151 | 21.567 | −12.882 | 1.00 | 25.12 | H | C |
| ATOM | 375 | CD1 | LEU | H | 50 | 9.377 | 23.031 | −13.138 | 1.00 | 18.87 | H | C |
| ATOM | 376 | CD2 | LEU | H | 50 | 10.473 | 20.829 | −12.902 | 1.00 | 16.10 | H | C |
| ATOM | 377 | C | LEU | H | 50 | 6.486 | 22.909 | −12.084 | 1.00 | 31.66 | H | C |
| ATOM | 378 | O | LEU | H | 50 | 5.904 | 23.045 | −13.163 | 1.00 | 32.24 | H | O |
| ATOM | 379 | N | ILE | H | 51 | 6.820 | 23.927 | −11.299 | 1.00 | 33.53 | H | N |
| ATOM | 380 | CA | ILE | H | 51 | 6.599 | 25.317 | −11.666 | 1.00 | 33.53 | H | C |
| ATOM | 381 | CB | ILE | H | 51 | 5.450 | 25.967 | −10.877 | 1.00 | 28.06 | H | C |
| ATOM | 382 | CG2 | ILE | H | 51 | 5.671 | 25.851 | −9.386 | 1.00 | 34.04 | H | C |
| ATOM | 383 | CG1 | ILE | H | 51 | 5.325 | 27.433 | −11.287 | 1.00 | 37.77 | H | C |
| ATOM | 384 | CD1 | ILE | H | 51 | 3.920 | 27.991 | −11.175 | 1.00 | 26.11 | H | C |
| ATOM | 385 | C | ILE | H | 51 | 7.918 | 26.030 | −11.402 | 1.00 | 33.69 | H | C |
| ATOM | 386 | O | ILE | H | 51 | 8.439 | 25.992 | −10.292 | 1.00 | 40.30 | H | O |
| ATOM | 387 | N | ASP | H | 52 | 8.493 | 26.594 | −12.459 | 1.00 | 36.83 | H | N |
| ATOM | 388 | CA | ASP | H | 52 | 9.778 | 27.304 | −12.420 | 1.00 | 36.82 | H | C |
| ATOM | 389 | CB | ASP | H | 52 | 10.382 | 27.305 | −13.836 | 1.00 | 41.27 | H | C |
| ATOM | 390 | CG | ASP | H | 52 | 11.825 | 27.792 | −13.888 | 1.00 | 42.22 | H | C |
| ATOM | 391 | OD1 | ASP | H | 52 | 12.232 | 28.626 | −13.064 | 1.00 | 47.36 | H | O |
| ATOM | 392 | OD2 | ASP | H | 52 | 12.556 | 27.351 | −14.797 | 1.00 | 46.22 | H | O |
| ATOM | 393 | C | ASP | H | 52 | 9.598 | 28.737 | −11.917 | 1.00 | 36.96 | H | C |
| ATOM | 394 | O | ASP | H | 52 | 8.722 | 29.460 | −12.385 | 1.00 | 37.60 | H | O |
| ATOM | 395 | N | PRO | H | 52A | 10.414 | 29.149 | −10.932 | 1.00 | 37.27 | H | N |
| ATOM | 396 | CD | PRO | H | 52A | 11.354 | 28.277 | −10.204 | 1.00 | 36.53 | H | C |
| ATOM | 397 | CA | PRO | H | 52A | 10.390 | 30.487 | −10.325 | 1.00 | 41.08 | H | C |
| ATOM | 398 | CB | PRO | H | 52A | 11.248 | 30.299 | −9.077 | 1.00 | 38.49 | H | C |
| ATOM | 399 | CG | PRO | H | 52A | 12.260 | 29.285 | −9.530 | 1.00 | 34.86 | H | C |
| ATOM | 400 | C | PRO | H | 52A | 10.987 | 31.600 | −11.190 | 1.00 | 45.13 | H | C |
| ATOM | 401 | O | PRO | H | 52A | 10.644 | 32.768 | −11.015 | 1.00 | 49.07 | H | O |
| ATOM | 402 | N | GLU | H | 53 | 11.893 | 31.223 | −12.094 | 1.00 | 45.80 | H | N |
| ATOM | 403 | CA | GLU | H | 53 | 12.603 | 32.148 | −12.994 | 1.00 | 49.26 | H | C |
| ATOM | 404 | CB | GLU | H | 53 | 13.728 | 31.387 | −13.732 | 1.00 | 51.31 | H | C |
| ATOM | 405 | CG | GLU | H | 53 | 15.128 | 32.043 | −13.714 | 1.00 | 55.92 | H | C |
| ATOM | 406 | CD | GLU | H | 53 | 16.254 | 31.071 | −14.138 | 1.00 | 54.87 | H | C |
| ATOM | 407 | OE1 | GLU | H | 53 | 16.670 | 30.226 | −13.307 | 1.00 | 56.96 | H | O |
| ATOM | 408 | OE2 | GLU | H | 53 | 16.731 | 31.157 | −15.294 | 1.00 | 51.97 | H | O |
| ATOM | 409 | C | GLU | H | 53 | 11.715 | 32.883 | −14.009 | 1.00 | 48.33 | H | C |
| ATOM | 410 | O | GLU | H | 53 | 11.894 | 34.083 | −14.235 | 1.00 | 51.41 | H | O |
| ATOM | 411 | N | GLN | H | 54 | 10.768 | 32.168 | −14.618 | 1.00 | 44.64 | H | N |
| ATOM | 412 | CA | GLN | H | 54 | 9.874 | 32.772 | −15.608 | 1.00 | 45.13 | H | C |
| ATOM | 413 | CB | GLN | H | 54 | 10.437 | 32.604 | −17.028 | 1.00 | 41.31 | H | C |
| ATOM | 414 | CG | GLN | H | 54 | 10.374 | 31.203 | −17.600 | 1.00 | 39.75 | H | C |
| ATOM | 415 | CD | GLN | H | 54 | 11.158 | 30.192 | −16.798 | 1.00 | 40.79 | H | C |
| ATOM | 416 | OE1 | GLN | H | 54 | 10.582 | 29.423 | −16.040 | 1.00 | 39.71 | H | O |
| ATOM | 417 | NE2 | GLN | H | 54 | 12.475 | 30.174 | −16.975 | 1.00 | 43.41 | H | N |
| ATOM | 418 | C | GLN | H | 54 | 8.416 | 32.319 | −15.570 | 1.00 | 41.83 | H | C |
| ATOM | 419 | O | GLN | H | 54 | 7.587 | 32.851 | −16.305 | 1.00 | 50.71 | H | O |
| ATOM | 420 | N | GLY | H | 55 | 8.098 | 31.350 | −14.720 | 1.00 | 40.77 | H | N |
| ATOM | 421 | CA | GLY | H | 55 | 6.724 | 30.891 | −14.616 | 1.00 | 38.45 | H | C |
| ATOM | 422 | C | GLY | H | 55 | 6.386 | 29.657 | −15.420 | 1.00 | 36.63 | H | C |
| ATOM | 423 | O | GLY | H | 55 | 5.228 | 29.254 | −15.457 | 1.00 | 37.44 | H | O |
| ATOM | 424 | N | ASN | H | 56 | 7.388 | 29.058 | −16.060 | 1.00 | 38.00 | H | N |
| ATOM | 425 | CA | ASN | H | 56 | 7.180 | 27.854 | −16.861 | 1.00 | 36.27 | H | C |
| ATOM | 426 | CB | ASN | H | 56 | 8.500 | 27.317 | −17.417 | 1.00 | 31.99 | H | C |
| ATOM | 427 | CG | ASN | H | 56 | 8.991 | 28.098 | −18.593 | 1.00 | 31.55 | H | C |
| ATOM | 428 | OD1 | ASN | H | 56 | 8.306 | 28.999 | −19.083 | 1.00 | 32.92 | H | O |
| ATOM | 429 | ND2 | ASN | H | 56 | 10.193 | 27.779 | −19.051 | 1.00 | 28.48 | H | N |
| ATOM | 430 | C | ASN | H | 56 | 6.560 | 26.757 | −16.037 | 1.00 | 36.61 | H | C |
| ATOM | 431 | O | ASN | H | 56 | 6.810 | 26.655 | −14.844 | 1.00 | 42.45 | H | O |
| ATOM | 432 | N | THR | H | 57 | 5.745 | 25.941 | −16.687 | 1.00 | 35.15 | H | N |
| ATOM | 433 | CA | THR | H | 57 | 5.107 | 24.816 | −16.044 | 1.00 | 32.72 | H | C |
| ATOM | 434 | CB | THR | H | 57 | 3.590 | 25.048 | −15.875 | 1.00 | 37.18 | H | C |
| ATOM | 435 | OG1 | THR | H | 57 | 3.072 | 25.718 | −17.031 | 1.00 | 42.76 | H | O |
| ATOM | 436 | CG2 | THR | H | 57 | 3.313 | 25.894 | −14.642 | 1.00 | 32.58 | H | C |
| ATOM | 437 | C | THR | H | 57 | 5.396 | 23.624 | −16.949 | 1.00 | 32.18 | H | C |
| ATOM | 438 | O | THR | H | 57 | 5.192 | 23.698 | −18.155 | 1.00 | 39.45 | H | O |
| ATOM | 439 | N | ILE | H | 58 | 6.000 | 22.589 | −16.377 | 1.00 | 26.81 | H | N |
| ATOM | 440 | CA | ILE | H | 58 | 6.355 | 21.386 | −17.093 | 1.00 | 23.81 | H | C |
| ATOM | 441 | CB | ILE | H | 58 | 7.895 | 21.180 | −17.079 | 1.00 | 30.26 | H | C |
| ATOM | 442 | CG2 | ILE | H | 58 | 8.270 | 19.862 | −17.723 | 1.00 | 31.44 | H | C |
| ATOM | 443 | CG1 | ILE | H | 58 | 8.587 | 22.264 | −17.906 | 1.00 | 34.94 | H | C |
| ATOM | 444 | CD1 | ILE | H | 58 | 8.614 | 23.606 | −17.272 | 1.00 | 35.53 | H | C |
| ATOM | 445 | C | ILE | H | 58 | 5.641 | 20.195 | −16.443 | 1.00 | 31.05 | H | C |
| ATOM | 446 | O | ILE | H | 58 | 5.604 | 20.067 | −15.214 | 1.00 | 34.04 | H | O |
| ATOM | 447 | N | TYR | H | 59 | 5.033 | 19.345 | −17.262 | 1.00 | 29.03 | H | N |
| ATOM | 448 | CA | TYR | H | 59 | 4.320 | 18.194 | −16.731 | 1.00 | 31.13 | H | C |
| ATOM | 449 | CB | TYR | H | 59 | 2.813 | 18.361 | −16.906 | 1.00 | 24.95 | H | C |
| ATOM | 450 | CG | TYR | H | 59 | 2.288 | 19.721 | −16.581 | 1.00 | 22.93 | H | C |
| ATOM | 451 | CD1 | TYR | H | 59 | 2.274 | 20.724 | −17.542 | 1.00 | 22.67 | H | C |
| ATOM | 452 | CE1 | TYR | H | 59 | 1.764 | 21.992 | −17.257 | 1.00 | 23.33 | H | C |
| ATOM | 453 | CD2 | TYR | H | 59 | 1.781 | 20.011 | −15.317 | 1.00 | 24.18 | H | C |
| ATOM | 454 | CE2 | TYR | H | 59 | 1.269 | 21.288 | −15.021 | 1.00 | 25.18 | H | C |
| ATOM | 455 | CZ | TYR | H | 59 | 1.267 | 22.268 | −15.998 | 1.00 | 21.50 | H | C |
| ATOM | 456 | OH | TYR | H | 59 | 0.790 | 23.531 | −15.712 | 1.00 | 26.89 | H | O |
| ATOM | 457 | C | TYR | H | 59 | 4.715 | 16.926 | −17.441 | 1.00 | 26.64 | H | C |
| ATOM | 458 | O | TYR | H | 59 | 5.334 | 16.968 | −18.497 | 1.00 | 30.58 | H | O |
| ATOM | 459 | N | ASP | H | 60 | 4.380 | 15.799 | −16.829 | 1.00 | 27.15 | H | N |
| ATOM | 460 | CA | ASP | H | 60 | 4.627 | 14.504 | −17.447 | 1.00 | 33.22 | H | C |
| ATOM | 461 | CB | ASP | H | 60 | 4.313 | 13.373 | −16.465 | 1.00 | 25.54 | H | C |
| ATOM | 462 | CG | ASP | H | 60 | 4.576 | 11.993 | −17.053 | 1.00 | 31.16 | H | C |
| ATOM | 463 | OD1 | ASP | H | 60 | 4.495 | 11.826 | −18.289 | 1.00 | 28.89 | H | O |
| ATOM | 464 | OD2 | ASP | H | 60 | 4.876 | 11.063 | −16.270 | 1.00 | 33.69 | H | O |
| ATOM | 465 | C | ASP | H | 60 | 3.600 | 14.465 | −18.578 | 1.00 | 33.15 | H | C |
| ATOM | 466 | O | ASP | H | 60 | 2.430 | 14.795 | −18.358 | 1.00 | 28.28 | H | O |
| ATOM | 467 | N | PRO | H | 61 | 4.040 | 14.152 | −19.809 | 1.00 | 36.92 | H | N |
| ATOM | 468 | CD | PRO | H | 61 | 5.450 | 14.052 | −20.226 | 1.00 | 41.13 | H | C |
| ATOM | 469 | CA | PRO | H | 61 | 3.154 | 14.072 | −20.977 | 1.00 | 40.09 | H | C |
| ATOM | 470 | CB | PRO | H | 61 | 4.078 | 13.511 | −22.053 | 1.00 | 37.89 | H | C |
| ATOM | 471 | CG | PRO | H | 61 | 5.357 | 14.204 | −21.744 | 1.00 | 41.98 | H | C |
| ATOM | 472 | C | PRO | H | 61 | 1.956 | 13.153 | −20.720 | 1.00 | 40.40 | H | C |
| ATOM | 473 | O | PRO | H | 61 | 0.866 | 13.403 | −21.229 | 1.00 | 42.36 | H | O |
| ATOM | 474 | N | LYS | H | 62 | 2.166 | 12.127 | −19.894 | 1.00 | 39.60 | H | N |
| ATOM | 475 | CA | LYS | H | 62 | 1.130 | 11.161 | −19.516 | 1.00 | 42.07 | H | C |
| ATOM | 476 | CB | LYS | H | 62 | 1.560 | 10.374 | −18.277 | 1.00 | 41.85 | H | C |
| ATOM | 477 | CG | LYS | H | 62 | 2.469 | 9.193 | −18.517 | 1.00 | 51.28 | H | C |
| ATOM | 478 | CD | LYS | H | 62 | 3.130 | 8.730 | −17.199 | 1.00 | 51.15 | H | C |
| ATOM | 479 | CE | LYS | H | 62 | 2.211 | 8.906 | −15.983 | 1.00 | 48.93 | H | C |
| ATOM | 480 | NZ | LYS | H | 62 | 2.835 | 8.392 | −14.731 | 1.00 | 48.06 | H | N |
| ATOM | 481 | C | LYS | H | 62 | −0.184 | 11.837 | −19.163 | 1.00 | 44.26 | H | C |
| ATOM | 482 | O | LYS | H | 62 | −1.264 | 11.297 | −19.433 | 1.00 | 45.09 | H | O |
| ATOM | 483 | N | PHE | H | 63 | −0.082 | 13.001 | −18.525 | 1.00 | 43.09 | H | N |
| ATOM | 484 | CA | PHE | H | 63 | −1.261 | 13.738 | −18.091 | 1.00 | 46.97 | H | C |
| ATOM | 485 | CB | PHE | H | 63 | −0.917 | 14.638 | −16.894 | 1.00 | 41.28 | H | C |
| ATOM | 486 | CG | PHE | H | 63 | −0.502 | 13.862 | −15.662 | 1.00 | 38.95 | H | C |
| ATOM | 487 | CD1 | PHE | H | 63 | −1.420 | 13.056 | −14.988 | 1.00 | 34.76 | H | C |
| ATOM | 488 | CD2 | PHE | H | 63 | 0.819 | 13.870 | −15.227 | 1.00 | 35.21 | H | C |
| ATOM | 489 | CE1 | PHE | H | 63 | −1.025 | 12.272 | −13.920 | 1.00 | 31.44 | H | C |
| ATOM | 490 | CE2 | PHE | H | 63 | 1.221 | 13.086 | −14.157 | 1.00 | 29.89 | H | C |
| ATOM | 491 | CZ | PHE | H | 63 | 0.297 | 12.284 | −13.504 | 1.00 | 34.19 | H | C |
| ATOM | 492 | C | PHE | H | 63 | −1.981 | 14.516 | −19.180 | 1.00 | 52.49 | H | C |
| ATOM | 493 | O | PHE | H | 63 | −3.050 | 15.078 | −18.931 | 1.00 | 59.01 | H | O |
| ATOM | 494 | N | GLN | H | 64 | −1.424 | 14.490 | −20.392 | 1.00 | 54.79 | H | N |
| ATOM | 495 | CA | GLN | H | 64 | −1.970 | 15.185 | −21.568 | 1.00 | 55.90 | H | C |
| ATOM | 496 | CB | GLN | H | 64 | −2.578 | 14.180 | −22.578 | 1.00 | 58.51 | H | C |
| ATOM | 497 | CG | GLN | H | 64 | −3.655 | 13.220 | −22.050 | 1.00 | 60.39 | H | C |
| ATOM | 498 | CD | GLN | H | 64 | −3.939 | 12.049 | −23.009 | 1.00 | 66.37 | H | C |
| ATOM | 499 | OE1 | GLN | H | 64 | −3.510 | 12.058 | −24.169 | 1.00 | 64.85 | H | O |
| ATOM | 500 | NE2 | GLN | H | 64 | −4.659 | 11.035 | −22.516 | 1.00 | 63.03 | H | N |
| ATOM | 501 | C | GLN | H | 64 | −2.902 | 16.396 | −21.326 | 1.00 | 55.28 | H | C |
| ATOM | 502 | O | GLN | H | 64 | −4.123 | 16.317 | −21.508 | 1.00 | 56.99 | H | O |
| ATOM | 503 | N | ASP | H | 65 | −2.302 | 17.502 | −20.888 | 1.00 | 48.58 | H | N |
| ATOM | 504 | CA | ASP | H | 65 | −2.998 | 18.758 | −20.614 | 1.00 | 46.29 | H | C |
| ATOM | 505 | CB | ASP | H | 65 | −3.497 | 19.414 | −21.922 | 1.00 | 46.17 | H | C |
| ATOM | 506 | CG | ASP | H | 65 | −4.876 | 18.920 | −22.366 | 0.00 | 43.30 | H | C |
| ATOM | 507 | OD1 | ASP | H | 65 | −5.896 | 19.396 | −21.820 | 0.00 | 41.91 | H | O |
| ATOM | 508 | OD2 | ASP | H | 65 | −4.942 | 18.083 | −23.291 | 0.00 | 42.00 | H | O |
| ATOM | 509 | C | ASP | H | 65 | −4.091 | 18.770 | −19.534 | 1.00 | 45.86 | H | C |
| ATOM | 510 | O | ASP | H | 65 | −4.757 | 19.789 | −19.349 | 1.00 | 49.49 | H | O |
| ATOM | 511 | N | ARG | H | 66 | −4.277 | 17.664 | −18.818 | 1.00 | 38.82 | H | N |
| ATOM | 512 | CA | ARG | H | 66 | −5.281 | 17.632 | −17.752 | 1.00 | 38.96 | H | C |
| ATOM | 513 | CB | ARG | H | 66 | −5.679 | 16.201 | −17.410 | 1.00 | 38.82 | H | C |
| ATOM | 514 | CG | ARG | H | 66 | −6.703 | 15.591 | −18.330 | 1.00 | 35.93 | H | C |
| ATOM | 515 | CD | ARG | H | 66 | −6.442 | 14.111 | −18.443 | 1.00 | 33.93 | H | C |
| ATOM | 516 | NE | ARG | H | 66 | −6.355 | 13.455 | −17.147 | 1.00 | 32.12 | H | N |
| ATOM | 517 | CZ | ARG | H | 66 | −5.580 | 12.410 | −16.903 | 1.00 | 33.55 | H | C |
| ATOM | 518 | NH1 | ARG | H | 66 | −4.817 | 11.910 | −17.868 | 1.00 | 44.62 | H | N |
| ATOM | 519 | NH2 | ARG | H | 66 | −5.601 | 11.833 | −15.715 | 1.00 | 37.01 | H | N |
| ATOM | 520 | C | ARG | H | 66 | −4.785 | 18.307 | −16.472 | 1.00 | 39.96 | H | C |
| ATOM | 521 | O | ARG | H | 66 | −5.587 | 18.770 | −15.665 | 1.00 | 40.88 | H | O |
| ATOM | 522 | N | ALA | H | 67 | −3.468 | 18.336 | −16.278 | 1.00 | 38.78 | H | N |
| ATOM | 523 | CA | ALA | H | 67 | −2.881 | 18.943 | −15.083 | 1.00 | 40.42 | H | C |
| ATOM | 524 | CB | ALA | H | 67 | −1.729 | 18.105 | −14.573 | 1.00 | 38.25 | H | C |
| ATOM | 525 | C | ALA | H | 67 | −2.418 | 20.375 | −15.268 | 1.00 | 39.76 | H | C |
| ATOM | 526 | O | ALA | H | 67 | −1.718 | 20.702 | −16.225 | 1.00 | 44.29 | H | O |
| ATOM | 527 | N | THR | H | 68 | −2.795 | 21.223 | −14.322 | 1.00 | 42.39 | H | N |
| ATOM | 528 | CA | THR | H | 68 | −2.420 | 22.622 | −14.348 | 1.00 | 37.35 | H | C |
| ATOM | 529 | CB | THR | H | 68 | −3.633 | 23.523 | −14.686 | 1.00 | 35.37 | H | C |
| ATOM | 530 | OG1 | THR | H | 68 | −4.125 | 23.179 | −15.982 | 1.00 | 43.56 | H | O |
| ATOM | 531 | CG2 | THR | H | 68 | −3.239 | 24.989 | −14.702 | 1.00 | 36.01 | H | C |
| ATOM | 532 | C | THR | H | 68 | −1.866 | 22.986 | −12.977 | 1.00 | 38.63 | H | C |
| ATOM | 533 | O | THR | H | 68 | −2.568 | 22.906 | −11.970 | 1.00 | 42.52 | H | O |
| ATOM | 534 | N | ILE | H | 69 | −0.580 | 23.315 | −12.942 | 1.00 | 36.69 | H | N |
| ATOM | 535 | CA | ILE | H | 69 | 0.088 | 23.716 | −11.715 | 1.00 | 29.96 | H | C |
| ATOM | 536 | CB | ILE | H | 69 | 1.490 | 23.075 | −11.620 | 1.00 | 29.42 | H | C |
| ATOM | 537 | CG2 | ILE | H | 69 | 2.276 | 23.672 | −10.462 | 1.00 | 26.98 | H | C |
| ATOM | 538 | CG1 | ILE | H | 69 | 1.366 | 21.561 | −11.450 | 1.00 | 24.17 | H | C |
| ATOM | 539 | CD1 | ILE | H | 69 | 2.689 | 20.834 | −11.520 | 1.00 | 25.22 | H | C |
| ATOM | 540 | C | ILE | H | 69 | 0.208 | 25.243 | −11.737 | 1.00 | 36.74 | H | C |
| ATOM | 541 | O | ILE | H | 69 | 0.596 | 25.833 | −12.754 | 1.00 | 36.24 | H | O |
| ATOM | 542 | N | SER | H | 70 | −0.156 | 25.878 | −10.625 | 1.00 | 37.01 | H | N |
| ATOM | 543 | CA | SER | H | 70 | −0.084 | 27.326 | −10.511 | 1.00 | 37.01 | H | C |
| ATOM | 544 | CB | SER | H | 70 | −1.435 | 27.954 | −10.879 | 1.00 | 38.50 | H | C |
| ATOM | 545 | OG | SER | H | 70 | −2.444 | 27.646 | −9.929 | 1.00 | 42.59 | H | O |
| ATOM | 546 | C | SER | H | 70 | 0.348 | 27.741 | −9.096 | 1.00 | 42.97 | H | C |
| ATOM | 547 | O | SER | H | 70 | 0.604 | 26.892 | −8.236 | 1.00 | 41.20 | H | O |
| ATOM | 548 | N | ALA | H | 71 | 0.422 | 29.049 | −8.859 | 1.00 | 41.73 | H | N |
| ATOM | 549 | CA | ALA | H | 71 | 0.836 | 29.556 | −7.562 | 1.00 | 43.49 | H | C |
| ATOM | 550 | CB | ALA | H | 71 | 2.352 | 29.431 | −7.416 | 1.00 | 40.68 | H | C |
| ATOM | 551 | C | ALA | H | 71 | 0.416 | 30.997 | −7.344 | 1.00 | 40.46 | H | C |
| ATOM | 552 | O | ALA | H | 71 | 0.334 | 31.783 | −8.280 | 1.00 | 42.94 | H | O |
| ATOM | 553 | N | ASP | H | 72 | 0.142 | 31.328 | −6.092 | 1.00 | 44.63 | H | N |
| ATOM | 554 | CA | ASP | H | 72 | −0.237 | 32.677 | −5.708 | 1.00 | 45.26 | H | C |
| ATOM | 555 | CB | ASP | H | 72 | −1.627 | 32.694 | −5.076 | 1.00 | 47.24 | H | C |
| ATOM | 556 | CG | ASP | H | 72 | −2.100 | 34.103 | −4.724 | 1.00 | 52.45 | H | C |
| ATOM | 557 | OD1 | ASP | H | 72 | −1.280 | 35.048 | −4.756 | 1.00 | 51.58 | H | O |
| ATOM | 558 | OD2 | ASP | H | 72 | −3.307 | 34.264 | −4.424 | 1.00 | 55.92 | H | O |
| ATOM | 559 | C | ASP | H | 72 | 0.782 | 33.120 | −4.681 | 1.00 | 45.15 | H | C |
| ATOM | 560 | O | ASP | H | 72 | 0.762 | 32.653 | −3.541 | 1.00 | 48.90 | H | O |
| ATOM | 561 | N | ASN | H | 73 | 1.674 | 34.013 | −5.085 | 1.00 | 42.40 | H | N |
| ATOM | 562 | CA | ASN | H | 73 | 2.695 | 34.503 | −4.178 | 1.00 | 42.62 | H | C |
| ATOM | 563 | CB | ASN | H | 73 | 3.738 | 35.314 | −4.941 | 1.00 | 43.44 | H | C |
| ATOM | 564 | CG | ASN | H | 73 | 4.579 | 34.449 | −5.876 | 1.00 | 46.76 | H | C |
| ATOM | 565 | OD1 | ASN | H | 73 | 5.370 | 34.961 | −6.659 | 1.00 | 50.55 | H | O |
| ATOM | 566 | ND2 | ASN | H | 73 | 4.418 | 33.133 | −5.785 | 1.00 | 51.83 | H | N |
| ATOM | 567 | C | ASN | H | 73 | 2.133 | 35.284 | −2.989 | 1.00 | 44.68 | H | C |
| ATOM | 568 | O | ASN | H | 73 | 2.694 | 35.215 | −1.895 | 1.00 | 49.02 | H | O |
| ATOM | 569 | N | SER | H | 74 | 1.004 | 35.971 | −3.177 | 1.00 | 42.21 | H | N |
| ATOM | 570 | CA | SER | H | 74 | 0.408 | 36.740 | −2.086 | 1.00 | 46.06 | H | C |
| ATOM | 571 | CB | SER | H | 74 | −0.669 | 37.706 | −2.617 | 1.00 | 46.86 | H | C |
| ATOM | 572 | OG | SER | H | 74 | −1.805 | 37.015 | −3.109 | 1.00 | 47.41 | H | O |
| ATOM | 573 | C | SER | H | 74 | −0.138 | 35.815 | −0.979 | 1.00 | 44.93 | H | C |
| ATOM | 574 | O | SER | H | 74 | −0.258 | 36.216 | 0.184 | 1.00 | 47.58 | H | O |
| ATOM | 575 | N | LYS | H | 75 | −0.456 | 34.578 | −1.344 | 1.00 | 39.52 | H | N |
| ATOM | 576 | CA | LYS | H | 75 | −0.949 | 33.608 | −0.379 | 1.00 | 35.66 | H | C |
| ATOM | 577 | CB | LYS | H | 75 | −2.202 | 32.900 | −0.902 | 1.00 | 35.95 | H | C |
| ATOM | 578 | CG | LYS | H | 75 | −3.436 | 33.773 | −0.978 | 1.00 | 40.83 | H | C |
| ATOM | 579 | CD | LYS | H | 75 | −4.681 | 32.930 | −1.179 | 1.00 | 44.55 | H | C |
| ATOM | 580 | CE | LYS | H | 75 | −5.941 | 33.730 | −0.866 | 1.00 | 54.16 | H | C |
| ATOM | 581 | NZ | LYS | H | 75 | −7.089 | 32.847 | −0.474 | 1.00 | 54.55 | H | N |
| ATOM | 582 | C | LYS | H | 75 | 0.146 | 32.589 | −0.060 | 1.00 | 32.68 | H | C |
| ATOM | 583 | O | LYS | H | 75 | −0.019 | 31.746 | 0.816 | 1.00 | 34.82 | H | O |
| ATOM | 584 | N | ASN | H | 76 | 1.273 | 32.699 | −0.760 | 1.00 | 31.95 | H | N |
| ATOM | 585 | CA | ASN | H | 76 | 2.426 | 31.808 | −0.586 | 1.00 | 30.15 | H | C |
| ATOM | 586 | CB | ASN | H | 76 | 3.130 | 32.076 | 0.751 | 1.00 | 19.47 | H | C |
| ATOM | 587 | CG | ASN | H | 76 | 4.610 | 31.771 | 0.690 | 1.00 | 19.32 | H | C |
| ATOM | 588 | OD1 | ASN | H | 76 | 5.287 | 32.162 | −0.247 | 1.00 | 32.55 | H | O |
| ATOM | 589 | ND2 | ASN | H | 76 | 5.114 | 31.051 | 1.675 | 1.00 | 26.13 | H | N |
| ATOM | 590 | C | ASN | H | 76 | 1.950 | 30.359 | −0.698 | 1.00 | 31.95 | H | C |
| ATOM | 591 | O | ASN | H | 76 | 2.392 | 29.460 | 0.035 | 1.00 | 24.15 | H | O |
| ATOM | 592 | N | THR | H | 77 | 1.045 | 30.164 | −1.658 | 1.00 | 33.40 | H | N |
| ATOM | 593 | CA | THR | H | 77 | 0.420 | 28.879 | −1.928 | 1.00 | 33.47 | H | C |
| ATOM | 594 | CB | THR | H | 77 | −1.067 | 28.978 | −1.695 | 1.00 | 29.30 | H | C |
| ATOM | 595 | OG1 | THR | H | 77 | −1.283 | 29.450 | −0.368 | 1.00 | 33.70 | H | O |
| ATOM | 596 | CG2 | THR | H | 77 | −1.748 | 27.615 | −1.885 | 1.00 | 31.42 | H | C |
| ATOM | 597 | C | THR | H | 77 | 0.611 | 28.416 | −3.361 | 1.00 | 34.78 | H | C |
| ATOM | 598 | O | THR | H | 77 | 0.565 | 29.222 | −4.295 | 1.00 | 31.82 | H | O |
| ATOM | 599 | N | ALA | H | 78 | 0.824 | 27.113 | −3.519 | 1.00 | 28.70 | H | N |
| ATOM | 600 | CA | ALA | H | 78 | 0.992 | 26.503 | −4.829 | 1.00 | 27.50 | H | C |
| ATOM | 601 | CB | ALA | H | 78 | 2.242 | 25.617 | −4.848 | 1.00 | 16.57 | H | C |
| ATOM | 602 | C | ALA | H | 78 | −0.266 | 25.668 | −5.047 | 1.00 | 27.02 | H | C |
| ATOM | 603 | O | ALA | H | 78 | −0.880 | 25.208 | −4.085 | 1.00 | 28.36 | H | O |
| ATOM | 604 | N | TYR | H | 79 | −0.673 | 25.496 | −6.298 | 1.00 | 27.15 | H | N |
| ATOM | 605 | CA | TYR | H | 79 | −1.874 | 24.717 | −6.581 | 1.00 | 28.41 | H | C |
| ATOM | 606 | CB | TYR | H | 79 | −2.998 | 25.636 | −7.034 | 1.00 | 29.49 | H | C |
| ATOM | 607 | CG | TYR | H | 79 | −3.355 | 26.741 | −6.075 | 1.00 | 31.42 | H | C |
| ATOM | 608 | CD1 | TYR | H | 79 | −4.221 | 26.509 | −5.014 | 1.00 | 31.81 | H | C |
| ATOM | 609 | CE1 | TYR | H | 79 | −4.590 | 27.527 | −4.160 | 1.00 | 31.06 | H | C |
| ATOM | 610 | CD2 | TYR | H | 79 | −2.865 | 28.033 | −6.254 | 1.00 | 32.79 | H | C |
| ATOM | 611 | CE2 | TYR | H | 79 | −3.235 | 29.067 | −5.398 | 1.00 | 27.86 | H | C |
| ATOM | 612 | CZ | TYR | H | 79 | −4.096 | 28.805 | −4.354 | 1.00 | 30.47 | H | C |
| ATOM | 613 | OH | TYR | H | 79 | −4.469 | 29.811 | −3.488 | 1.00 | 35.93 | H | O |
| ATOM | 614 | C | TYR | H | 79 | −1.695 | 23.639 | −7.643 | 1.00 | 29.30 | H | C |
| ATOM | 615 | O | TYR | H | 79 | −0.827 | 23.731 | −8.509 | 1.00 | 25.63 | H | O |
| ATOM | 616 | N | LEU | H | 80 | −2.500 | 22.589 | −7.533 | 1.00 | 26.65 | H | N |
| ATOM | 617 | CA | LEU | H | 80 | −2.492 | 21.509 | −8.511 | 1.00 | 29.23 | H | C |
| ATOM | 618 | CB | LEU | H | 80 | −1.889 | 20.207 | −7.967 | 1.00 | 24.06 | H | C |
| ATOM | 619 | CG | LEU | H | 80 | −2.044 | 19.003 | −8.924 | 1.00 | 23.73 | H | C |
| ATOM | 620 | CD1 | LEU | H | 80 | −1.538 | 19.322 | −10.328 | 1.00 | 16.18 | H | C |
| ATOM | 621 | CD2 | LEU | H | 80 | −1.331 | 17.772 | −8.379 | 1.00 | 16.28 | H | C |
| ATOM | 622 | C | LEU | H | 80 | −3.943 | 21.263 | −8.886 | 1.00 | 32.32 | H | C |
| ATOM | 623 | O | LEU | H | 80 | −4.790 | 21.000 | −8.021 | 1.00 | 24.53 | H | O |
| ATOM | 624 | N | GLN | H | 81 | −4.231 | 21.404 | −10.172 | 1.00 | 33.30 | H | N |
| ATOM | 625 | CA | GLN | H | 81 | −5.567 | 21.174 | −10.675 | 1.00 | 36.11 | H | C |
| ATOM | 626 | CB | GLN | H | 81 | −6.117 | 22.447 | −11.312 | 1.00 | 40.41 | H | C |
| ATOM | 627 | CG | GLN | H | 81 | −7.371 | 22.215 | −12.132 | 1.00 | 51.19 | H | C |
| ATOM | 628 | CD | GLN | H | 81 | −8.509 | 23.112 | −11.717 | 1.00 | 57.56 | H | C |
| ATOM | 629 | OE1 | GLN | H | 81 | −8.353 | 23.975 | −10.840 | 1.00 | 58.53 | H | O |
| ATOM | 630 | NE2 | GLN | H | 81 | −9.673 | 22.916 | −12.343 | 1.00 | 55.98 | H | N |
| ATOM | 631 | C | GLN | H | 81 | −5.508 | 20.046 | −11.695 | 1.00 | 33.32 | H | C |
| ATOM | 632 | O | GLN | H | 81 | −4.576 | 19.977 | −12.492 | 1.00 | 31.89 | H | O |
| ATOM | 633 | N | MET | H | 82 | −6.486 | 19.150 | −11.647 | 1.00 | 31.48 | H | N |
| ATOM | 634 | CA | MET | H | 82 | −6.540 | 18.040 | −12.587 | 1.00 | 36.23 | H | C |
| ATOM | 635 | CB | MET | H | 82 | −5.873 | 16.799 | −12.003 | 1.00 | 38.58 | H | C |
| ATOM | 636 | CG | MET | H | 82 | −5.301 | 15.895 | −13.077 | 1.00 | 44.48 | H | C |
| ATOM | 637 | SD | MET | H | 82 | −4.377 | 14.495 | −12.455 | 1.00 | 49.16 | H | S |
| ATOM | 638 | CE | MET | H | 82 | −3.087 | 15.374 | −11.546 | 1.00 | 47.75 | H | C |
| ATOM | 639 | C | MET | H | 82 | −7.984 | 17.742 | −12.980 | 1.00 | 39.16 | H | C |
| ATOM | 640 | O | MET | H | 82 | −8.851 | 17.603 | −12.123 | 1.00 | 42.60 | H | O |
| ATOM | 641 | N | ASN | H | 82A | −8.233 | 17.669 | −14.287 | 1.00 | 39.87 | H | N |
| ATOM | 642 | CA | ASN | H | 82A | −9.567 | 17.417 | −14.826 | 1.00 | 35.46 | H | C |
| ATOM | 643 | CB | ASN | H | 82A | −9.851 | 18.412 | −15.944 | 1.00 | 38.03 | H | C |
| ATOM | 644 | CG | ASN | H | 82A | −9.538 | 19.843 | −15.543 | 1.00 | 41.17 | H | C |
| ATOM | 645 | OD1 | ASN | H | 82A | −9.953 | 20.318 | −14.478 | 1.00 | 41.38 | H | O |
| ATOM | 646 | ND2 | ASN | H | 82A | −8.798 | 20.540 | −16.398 | 1.00 | 41.71 | H | N |
| ATOM | 647 | C | ASN | H | 82A | −9.714 | 15.998 | −15.363 | 1.00 | 36.24 | H | C |
| ATOM | 648 | O | ASN | H | 82A | −8.743 | 15.243 | −15.414 | 1.00 | 34.17 | H | O |
| ATOM | 649 | N | SER | H | 82B | −10.938 | 15.638 | −15.749 | 1.00 | 35.08 | H | N |
| ATOM | 650 | CA | SER | H | 82B | −11.252 | 14.308 | −16.300 | 1.00 | 31.13 | H | C |
| ATOM | 651 | CB | SER | H | 82B | −10.852 | 14.237 | −17.780 | 1.00 | 28.26 | H | C |
| ATOM | 652 | OG | SER | H | 82B | −11.042 | 15.484 | −18.437 | 1.00 | 30.52 | H | O |
| ATOM | 653 | C | SER | H | 82B | −10.580 | 13.170 | −15.515 | 1.00 | 30.16 | H | C |
| ATOM | 654 | O | SER | H | 82B | −9.904 | 12.309 | −16.089 | 1.00 | 33.77 | H | O |
| ATOM | 655 | N | LEU | H | 82C | −10.783 | 13.172 | −14.202 | 1.00 | 25.98 | H | N |
| ATOM | 656 | CA | LEU | H | 82C | −10.191 | 12.178 | −13.326 | 1.00 | 24.56 | H | C |
| ATOM | 657 | CB | LEU | H | 82C | −10.467 | 12.526 | −11.866 | 1.00 | 27.87 | H | C |
| ATOM | 658 | CG | LEU | H | 82C | −9.717 | 13.786 | −11.445 | 1.00 | 27.77 | H | C |
| ATOM | 659 | CD1 | LEU | H | 82C | −9.922 | 14.060 | −9.984 | 1.00 | 31.28 | H | C |
| ATOM | 660 | CD2 | LEU | H | 82C | −8.266 | 13.591 | −11.729 | 1.00 | 17.27 | H | C |
| ATOM | 661 | C | LEU | H | 82C | −10.648 | 10.774 | −13.614 | 1.00 | 26.18 | H | C |
| ATOM | 662 | O | LEU | H | 82C | −11.804 | 10.556 | −13.962 | 1.00 | 32.85 | H | O |
| ATOM | 663 | N | ARG | H | 83 | −9.719 | 9.831 | −13.484 | 1.00 | 25.94 | H | N |
| ATOM | 664 | CA | ARG | H | 83 | −9.980 | 8.417 | −13.720 | 1.00 | 25.14 | H | C |
| ATOM | 665 | CB | ARG | H | 83 | −9.181 | 7.907 | −14.928 | 1.00 | 28.82 | H | C |
| ATOM | 666 | CG | ARG | H | 83 | −9.108 | 8.860 | −16.110 | 1.00 | 39.70 | H | C |
| ATOM | 667 | CD | ARG | H | 83 | −8.082 | 8.386 | −17.156 | 1.00 | 47.02 | H | C |
| ATOM | 668 | NE | ARG | H | 83 | −7.910 | 9.356 | −18.248 | 1.00 | 50.40 | H | N |
| ATOM | 669 | CZ | ARG | H | 83 | −6.737 | 9.666 | −18.803 | 1.00 | 50.33 | H | C |
| ATOM | 670 | NH1 | ARG | H | 83 | −5.622 | 9.083 | −18.374 | 1.00 | 52.47 | H | N |
| ATOM | 671 | NH2 | ARG | H | 83 | −6.670 | 10.568 | −19.777 | 1.00 | 53.22 | H | N |
| ATOM | 672 | C | ARG | H | 83 | −9.505 | 7.670 | −12.483 | 1.00 | 30.52 | H | C |
| ATOM | 673 | O | ARG | H | 83 | −8.747 | 8.216 | −11.682 | 1.00 | 32.39 | H | O |
| ATOM | 674 | N | ALA | H | 84 | −9.902 | 6.405 | −12.372 | 1.00 | 30.37 | H | N |
| ATOM | 675 | CA | ALA | H | 84 | −9.535 | 5.546 | −11.253 | 1.00 | 30.44 | H | C |
| ATOM | 676 | CB | ALA | H | 84 | −10.005 | 4.121 | −11.526 | 1.00 | 30.99 | H | C |
| ATOM | 677 | C | ALA | H | 84 | −8.033 | 5.545 | −10.937 | 1.00 | 34.19 | H | C |
| ATOM | 678 | O | ALA | H | 84 | −7.640 | 5.475 | −9.773 | 1.00 | 28.68 | H | O |
| ATOM | 679 | N | GLU | H | 85 | −7.200 | 5.578 | −11.973 | 1.00 | 38.24 | H | N |
| ATOM | 680 | CA | GLU | H | 85 | −5.747 | 5.575 | −11.793 | 1.00 | 46.59 | H | C |
| ATOM | 681 | CB | GLU | H | 85 | −5.040 | 5.303 | −13.127 | 1.00 | 49.06 | H | C |
| ATOM | 682 | CG | GLU | H | 85 | −5.377 | 6.325 | −14.207 | 1.00 | 61.42 | H | C |
| ATOM | 683 | CD | GLU | H | 85 | −4.568 | 6.169 | −15.497 | 1.00 | 65.28 | H | C |
| ATOM | 684 | OE1 | GLU | H | 85 | −3.399 | 5.712 | −15.440 | 1.00 | 65.33 | H | O |
| ATOM | 685 | OE2 | GLU | H | 85 | −5.108 | 6.541 | −16.570 | 1.00 | 68.14 | H | O |
| ATOM | 686 | C | GLU | H | 85 | −5.218 | 6.878 | −11.171 | 1.00 | 42.68 | H | C |
| ATOM | 687 | O | GLU | H | 85 | −4.036 | 6.985 | −10.870 | 1.00 | 40.40 | H | O |
| ATOM | 688 | N | ASP | H | 86 | −6.091 | 7.870 | −11.017 | 1.00 | 38.06 | H | N |
| ATOM | 689 | CA | ASP | H | 86 | −5.711 | 9.137 | −10.421 | 1.00 | 31.49 | H | C |
| ATOM | 690 | CB | ASP | H | 86 | −6.472 | 10.304 | −11.058 | 1.00 | 35.67 | H | C |
| ATOM | 691 | CG | ASP | H | 86 | −6.014 | 10.600 | −12.483 | 1.00 | 36.11 | H | C |
| ATOM | 692 | OD1 | ASP | H | 86 | −4.867 | 10.251 | −12.836 | 1.00 | 37.08 | H | O |
| ATOM | 693 | OD2 | ASP | H | 86 | −6.800 | 11.191 | −13.255 | 1.00 | 36.83 | H | O |
| ATOM | 694 | C | ASP | H | 86 | −5.950 | 9.109 | −8.923 | 1.00 | 31.23 | H | C |
| ATOM | 695 | O | ASP | H | 86 | −5.660 | 10.081 | −8.232 | 1.00 | 32.22 | H | O |
| ATOM | 696 | N | THR | H | 87 | −6.496 | 8.004 | −8.422 | 1.00 | 27.59 | H | N |
| ATOM | 697 | CA | THR | H | 87 | −6.726 | 7.867 | −6.986 | 1.00 | 26.23 | H | C |
| ATOM | 698 | CB | THR | H | 87 | −7.522 | 6.614 | −6.638 | 1.00 | 23.36 | H | C |
| ATOM | 699 | OG1 | THR | H | 87 | −8.906 | 6.829 | −6.951 | 1.00 | 28.58 | H | O |
| ATOM | 700 | CG2 | THR | H | 87 | −7.370 | 6.287 | −5.147 | 1.00 | 22.60 | H | C |
| ATOM | 701 | C | THR | H | 87 | −5.359 | 7.745 | −6.359 | 1.00 | 25.63 | H | C |
| ATOM | 702 | O | THR | H | 87 | −4.650 | 6.787 | −6.630 | 1.00 | 27.89 | H | O |
| ATOM | 703 | N | ALA | H | 88 | −5.009 | 8.691 | −5.489 | 1.00 | 28.25 | H | N |
| ATOM | 704 | CA | ALA | H | 88 | −3.689 | 8.690 | −4.870 | 1.00 | 23.97 | H | C |
| ATOM | 705 | CB | ALA | H | 88 | −2.623 | 8.908 | −5.958 | 1.00 | 20.47 | H | C |
| ATOM | 706 | C | ALA | H | 88 | −3.475 | 9.736 | −3.791 | 1.00 | 26.18 | H | C |
| ATOM | 707 | O | ALA | H | 88 | −4.287 | 10.648 | −3.597 | 1.00 | 26.71 | H | O |
| ATOM | 708 | N | VAL | H | 89 | −2.361 | 9.574 | −3.078 | 1.00 | 26.63 | H | N |
| ATOM | 709 | CA | VAL | H | 89 | −1.958 | 10.536 | −2.072 | 1.00 | 21.73 | H | C |
| ATOM | 710 | CB | VAL | H | 89 | −1.089 | 9.907 | −0.946 | 1.00 | 24.95 | H | C |
| ATOM | 711 | CG1 | VAL | H | 89 | −0.550 | 10.983 | −0.012 | 1.00 | 19.07 | H | C |
| ATOM | 712 | CG2 | VAL | H | 89 | −1.913 | 8.924 | −0.135 | 1.00 | 25.15 | H | C |
| ATOM | 713 | C | VAL | H | 89 | −1.112 | 11.463 | −2.926 | 1.00 | 21.94 | H | C |
| ATOM | 714 | O | VAL | H | 89 | −0.253 | 10.995 | −3.675 | 1.00 | 14.71 | H | O |
| ATOM | 715 | N | TYR | H | 90 | −1.472 | 12.746 | −2.919 | 1.00 | 17.23 | H | N |
| ATOM | 716 | CA | TYR | H | 90 | −0.772 | 13.778 | −3.676 | 1.00 | 17.05 | H | C |
| ATOM | 717 | CB | TYR | H | 90 | −1.772 | 14.710 | −4.387 | 1.00 | 20.05 | H | C |
| ATOM | 718 | CG | TYR | H | 90 | −2.394 | 14.115 | −5.613 | 1.00 | 16.40 | H | C |
| ATOM | 719 | CD1 | TYR | H | 90 | −3.471 | 13.251 | −5.513 | 1.00 | 18.69 | H | C |
| ATOM | 720 | CE1 | TYR | H | 90 | −3.985 | 12.629 | −6.622 | 1.00 | 25.14 | H | C |
| ATOM | 721 | CD2 | TYR | H | 90 | −1.853 | 14.351 | −6.865 | 1.00 | 24.33 | H | C |
| ATOM | 722 | CE2 | TYR | H | 90 | −2.362 | 13.734 | −7.994 | 1.00 | 30.22 | H | C |
| ATOM | 723 | CZ | TYR | H | 90 | −3.428 | 12.869 | −7.864 | 1.00 | 28.72 | H | C |
| ATOM | 724 | OH | TYR | H | 90 | −3.929 | 12.223 | −8.969 | 1.00 | 33.55 | H | O |
| ATOM | 725 | C | TYR | H | 90 | 0.093 | 14.614 | −2.739 | 1.00 | 20.59 | H | C |
| ATOM | 726 | O | TYR | H | 90 | −0.380 | 15.136 | −1.725 | 1.00 | 17.97 | H | O |
| ATOM | 727 | N | TYR | H | 91 | 1.366 | 14.737 | −3.089 | 1.00 | 27.56 | H | N |
| ATOM | 728 | CA | TYR | H | 91 | 2.313 | 15.519 | −2.307 | 1.00 | 23.72 | H | C |
| ATOM | 729 | CB | TYR | H | 91 | 3.562 | 14.733 | −1.950 | 1.00 | 14.99 | H | C |
| ATOM | 730 | CG | TYR | H | 91 | 3.328 | 13.547 | −1.088 | 1.00 | 15.84 | H | C |
| ATOM | 731 | CD1 | TYR | H | 91 | 3.123 | 13.679 | 0.281 | 1.00 | 16.38 | H | C |
| ATOM | 732 | CE1 | TYR | H | 91 | 2.946 | 12.550 | 1.089 | 1.00 | 17.72 | H | C |
| ATOM | 733 | CD2 | TYR | H | 91 | 3.348 | 12.278 | −1.633 | 1.00 | 17.68 | H | C |
| ATOM | 734 | CE2 | TYR | H | 91 | 3.170 | 11.153 | −0.843 | 1.00 | 18.44 | H | C |
| ATOM | 735 | CZ | TYR | H | 91 | 2.970 | 11.288 | 0.510 | 1.00 | 15.13 | H | C |
| ATOM | 736 | OH | TYR | H | 91 | 2.774 | 10.145 | 1.252 | 1.00 | 19.28 | H | O |
| ATOM | 737 | C | TYR | H | 91 | 2.768 | 16.720 | −3.067 | 1.00 | 25.61 | H | C |
| ATOM | 738 | O | TYR | H | 91 | 2.903 | 16.712 | −4.296 | 1.00 | 28.60 | H | O |
| ATOM | 739 | N | CYS | H | 92 | 3.095 | 17.724 | −2.281 | 1.00 | 33.43 | H | N |
| ATOM | 740 | CA | CYS | H | 92 | 3.596 | 18.995 | −2.746 | 1.00 | 31.78 | H | C |
| ATOM | 741 | C | CYS | H | 92 | 5.034 | 18.982 | −2.238 | 1.00 | 29.44 | H | C |
| ATOM | 742 | O | CYS | H | 92 | 5.272 | 18.643 | −1.078 | 1.00 | 29.01 | H | O |
| ATOM | 743 | CB | CYS | H | 92 | 2.785 | 20.071 | −2.048 | 1.00 | 39.04 | H | C |
| ATOM | 744 | SG | CYS | H | 92 | 3.388 | 21.766 | −2.180 | 1.00 | 54.60 | H | S |
| ATOM | 745 | N | ALA | H | 93 | 5.999 | 19.282 | −3.102 | 1.00 | 27.60 | H | N |
| ATOM | 746 | CA | ALA | H | 93 | 7.391 | 19.281 | −2.661 | 1.00 | 24.86 | H | C |
| ATOM | 747 | CB | ALA | H | 93 | 8.070 | 17.997 | −3.060 | 1.00 | 25.21 | H | C |
| ATOM | 748 | C | ALA | H | 93 | 8.213 | 20.478 | −3.128 | 1.00 | 25.92 | H | C |
| ATOM | 749 | O | ALA | H | 93 | 7.993 | 21.022 | −4.210 | 1.00 | 22.59 | H | O |
| ATOM | 750 | N | ARG | H | 94 | 9.156 | 20.882 | −2.280 | 1.00 | 21.72 | H | N |
| ATOM | 751 | CA | ARG | H | 94 | 10.047 | 22.003 | −2.557 | 1.00 | 25.31 | H | C |
| ATOM | 752 | CB | ARG | H | 94 | 10.355 | 22.807 | −1.278 | 1.00 | 25.10 | H | C |
| ATOM | 753 | CG | ARG | H | 94 | 10.861 | 24.233 | −1.544 | 1.00 | 28.37 | H | C |
| ATOM | 754 | CD | ARG | H | 94 | 11.244 | 24.987 | −0.274 | 1.00 | 23.82 | H | C |
| ATOM | 755 | NE | ARG | H | 94 | 12.465 | 24.461 | 0.327 | 1.00 | 26.22 | H | N |
| ATOM | 756 | CZ | ARG | H | 94 | 13.325 | 25.194 | 1.027 | 1.00 | 28.91 | H | C |
| ATOM | 757 | NH1 | ARG | H | 94 | 13.086 | 26.480 | 1.223 | 1.00 | 30.05 | H | N |
| ATOM | 758 | NH2 | ARG | H | 94 | 14.460 | 24.670 | 1.467 | 1.00 | 23.26 | H | N |
| ATOM | 759 | C | ARG | H | 94 | 11.331 | 21.434 | −3.124 | 1.00 | 19.66 | H | C |
| ATOM | 760 | O | ARG | H | 94 | 11.850 | 20.423 | −2.642 | 1.00 | 22.57 | H | O |
| ATOM | 761 | N | ASP | H | 95 | 11.846 | 22.095 | −4.147 | 1.00 | 23.96 | H | N |
| ATOM | 762 | CA | ASP | H | 95 | 13.067 | 21.656 | −4.808 | 1.00 | 24.45 | H | C |
| ATOM | 763 | CB | ASP | H | 95 | 12.752 | 21.523 | −6.320 | 1.00 | 24.66 | H | C |
| ATOM | 764 | CG | ASP | H | 95 | 13.999 | 21.566 | −7.269 | 1.00 | 31.75 | H | C |
| ATOM | 765 | OD1 | ASP | H | 95 | 15.172 | 21.806 | −6.892 | 1.00 | 27.05 | H | O |
| ATOM | 766 | OD2 | ASP | H | 95 | 13.762 | 21.372 | −8.480 | 1.00 | 31.00 | H | O |
| ATOM | 767 | C | ASP | H | 95 | 14.238 | 22.601 | −4.528 | 1.00 | 20.25 | H | C |
| ATOM | 768 | O | ASP | H | 95 | 14.094 | 23.824 | −4.552 | 1.00 | 17.50 | H | O |
| ATOM | 769 | N | THR | H | 96 | 15.385 | 21.995 | −4.242 | 1.00 | 15.84 | H | N |
| ATOM | 770 | CA | THR | H | 96 | 16.663 | 22.690 | −4.049 | 1.00 | 19.21 | H | C |
| ATOM | 771 | CB | THR | H | 96 | 17.124 | 22.751 | −2.553 | 1.00 | 18.08 | H | C |
| ATOM | 772 | OG1 | THR | H | 96 | 16.334 | 23.705 | −1.840 | 1.00 | 22.10 | H | O |
| ATOM | 773 | CG2 | THR | H | 96 | 18.571 | 23.170 | −2.451 | 1.00 | 13.81 | H | C |
| ATOM | 774 | C | THR | H | 96 | 17.603 | 21.767 | −4.820 | 1.00 | 18.97 | H | C |
| ATOM | 775 | O | THR | H | 96 | 17.638 | 20.559 | −4.562 | 1.00 | 21.26 | H | O |
| ATOM | 776 | N | ALA | H | 97 | 18.287 | 22.296 | −5.831 | 1.00 | 21.98 | H | N |
| ATOM | 777 | CA | ALA | H | 97 | 19.219 | 21.477 | −6.611 | 1.00 | 15.99 | H | C |
| ATOM | 778 | CB | ALA | H | 97 | 20.421 | 21.105 | −5.758 | 1.00 | 21.88 | H | C |
| ATOM | 779 | C | ALA | H | 97 | 18.602 | 20.208 | −7.209 | 1.00 | 19.21 | H | C |
| ATOM | 780 | O | ALA | H | 97 | 19.272 | 19.179 | −7.342 | 1.00 | 15.70 | H | O |
| ATOM | 781 | N | ALA | H | 98 | 17.322 | 20.284 | −7.560 | 1.00 | 18.14 | H | N |
| ATOM | 782 | CA | ALA | H | 98 | 16.588 | 19.178 | −8.172 | 1.00 | 14.57 | H | C |
| ATOM | 783 | CB | ALA | H | 98 | 17.277 | 18.695 | −9.456 | 1.00 | 15.03 | H | C |
| ATOM | 784 | C | ALA | H | 98 | 16.262 | 18.000 | −7.273 | 1.00 | 18.63 | H | C |
| ATOM | 785 | O | ALA | H | 98 | 15.918 | 16.928 | −7.761 | 1.00 | 24.92 | H | O |
| ATOM | 786 | N | TYR | H | 99 | 16.460 | 18.152 | −5.972 | 1.00 | 20.96 | H | N |
| ATOM | 787 | CA | TYR | H | 99 | 16.089 | 17.093 | −5.050 | 1.00 | 15.14 | H | C |
| ATOM | 788 | CB | TYR | H | 99 | 17.291 | 16.530 | −4.289 | 1.00 | 23.83 | H | C |
| ATOM | 789 | CG | TYR | H | 99 | 17.905 | 17.428 | −3.241 | 1.00 | 30.03 | H | C |
| ATOM | 790 | CD1 | TYR | H | 99 | 17.322 | 17.572 | −1.982 | 1.00 | 28.39 | H | C |
| ATOM | 791 | CE1 | TYR | H | 99 | 17.912 | 18.357 | −1.007 | 1.00 | 28.92 | H | C |
| ATOM | 792 | CD2 | TYR | H | 99 | 19.097 | 18.092 | −3.490 | 1.00 | 29.70 | H | C |
| ATOM | 793 | CE2 | TYR | H | 99 | 19.697 | 18.875 | −2.522 | 1.00 | 32.43 | H | C |
| ATOM | 794 | CZ | TYR | H | 99 | 19.101 | 19.009 | −1.285 | 1.00 | 30.07 | H | C |
| ATOM | 795 | OH | TYR | H | 99 | 19.683 | 19.826 | −0.345 | 1.00 | 29.43 | H | O |
| ATOM | 796 | C | TYR | H | 99 | 15.048 | 17.734 | −4.145 | 1.00 | 18.19 | H | C |
| ATOM | 797 | O | TYR | H | 99 | 15.016 | 18.953 | −4.011 | 1.00 | 17.15 | H | O |
| ATOM | 798 | N | PHE | H | 100 | 14.122 | 16.939 | −3.623 | 1.00 | 20.72 | H | N |
| ATOM | 799 | CA | PHE | H | 100 | 13.066 | 17.484 | −2.782 | 1.00 | 16.97 | H | C |
| ATOM | 800 | CB | PHE | H | 100 | 11.806 | 16.627 | −2.905 | 1.00 | 22.78 | H | C |
| ATOM | 801 | CG | PHE | H | 100 | 11.336 | 16.448 | −4.317 | 1.00 | 16.70 | H | C |
| ATOM | 802 | CD1 | PHE | H | 100 | 10.998 | 17.541 | −5.090 | 1.00 | 22.29 | H | C |
| ATOM | 803 | CD2 | PHE | H | 100 | 11.286 | 15.188 | −4.892 | 1.00 | 19.02 | H | C |
| ATOM | 804 | CE1 | PHE | H | 100 | 10.622 | 17.386 | −6.421 | 1.00 | 21.59 | H | C |
| ATOM | 805 | CE2 | PHE | H | 100 | 10.914 | 15.023 | −6.219 | 1.00 | 12.81 | H | C |
| ATOM | 806 | CZ | PHE | H | 100 | 10.584 | 16.124 | −6.984 | 1.00 | 20.35 | H | C |
| ATOM | 807 | C | PHE | H | 100 | 13.504 | 17.598 | −1.333 | 1.00 | 18.56 | H | C |
| ATOM | 808 | O | PHE | H | 100 | 13.707 | 16.599 | −0.656 | 1.00 | 17.98 | H | O |
| ATOM | 809 | N | ASP | H | 101 | 13.653 | 18.822 | −0.854 | 1.00 | 21.54 | H | N |
| ATOM | 810 | CA | ASP | H | 101 | 14.079 | 19.019 | 0.522 | 1.00 | 24.23 | H | C |
| ATOM | 811 | CB | ASP | H | 101 | 15.067 | 20.198 | 0.628 | 1.00 | 22.46 | H | C |
| ATOM | 812 | CG | ASP | H | 101 | 14.495 | 21.528 | 0.113 | 1.00 | 26.26 | H | C |
| ATOM | 813 | OD1 | ASP | H | 101 | 13.294 | 21.621 | −0.212 | 1.00 | 30.16 | H | O |
| ATOM | 814 | OD2 | ASP | H | 101 | 15.265 | 22.508 | 0.064 | 1.00 | 25.86 | H | O |
| ATOM | 815 | C | ASP | H | 101 | 12.947 | 19.103 | 1.560 | 1.00 | 26.86 | H | C |
| ATOM | 816 | O | ASP | H | 101 | 13.171 | 18.856 | 2.752 | 1.00 | 33.09 | H | O |
| ATOM | 817 | N | TYR | H | 102 | 11.745 | 19.475 | 1.130 | 1.00 | 24.68 | H | N |
| ATOM | 818 | CA | TYR | H | 102 | 10.616 | 19.537 | 2.054 | 1.00 | 25.04 | H | C |
| ATOM | 819 | CB | TYR | H | 102 | 10.420 | 20.965 | 2.590 | 1.00 | 27.92 | H | C |
| ATOM | 820 | CG | TYR | H | 102 | 11.498 | 21.365 | 3.579 | 1.00 | 31.84 | H | C |
| ATOM | 821 | CD1 | TYR | H | 102 | 11.357 | 21.109 | 4.948 | 1.00 | 28.83 | H | C |
| ATOM | 822 | CE1 | TYR | H | 102 | 12.383 | 21.397 | 5.839 | 1.00 | 26.09 | H | C |
| ATOM | 823 | CD2 | TYR | H | 102 | 12.687 | 21.926 | 3.140 | 1.00 | 29.04 | H | C |
| ATOM | 824 | CE2 | TYR | H | 102 | 13.711 | 22.213 | 4.018 | 1.00 | 32.38 | H | C |
| ATOM | 825 | CZ | TYR | H | 102 | 13.561 | 21.944 | 5.362 | 1.00 | 31.83 | H | C |
| ATOM | 826 | OH | TYR | H | 102 | 14.623 | 22.176 | 6.202 | 1.00 | 31.18 | H | O |
| ATOM | 827 | C | TYR | H | 102 | 9.357 | 19.018 | 1.402 | 1.00 | 22.75 | H | C |
| ATOM | 828 | O | TYR | H | 102 | 9.143 | 19.224 | 0.219 | 1.00 | 25.56 | H | O |
| ATOM | 829 | N | TRP | H | 103 | 8.508 | 18.362 | 2.185 | 1.00 | 27.00 | H | N |
| ATOM | 830 | CA | TRP | H | 103 | 7.268 | 17.799 | 1.667 | 1.00 | 17.07 | H | C |
| ATOM | 831 | CB | TRP | H | 103 | 7.352 | 16.266 | 1.646 | 1.00 | 17.02 | H | C |
| ATOM | 832 | CG | TRP | H | 103 | 8.427 | 15.678 | 0.775 | 1.00 | 23.67 | H | C |
| ATOM | 833 | CD2 | TRP | H | 103 | 8.242 | 14.892 | −0.415 | 1.00 | 24.32 | H | C |
| ATOM | 834 | CE2 | TRP | H | 103 | 9.524 | 14.506 | −0.865 | 1.00 | 28.29 | H | C |
| ATOM | 835 | CE3 | TRP | H | 103 | 7.115 | 14.468 | −1.138 | 1.00 | 26.61 | H | C |
| ATOM | 836 | CD1 | TRP | H | 103 | 9.775 | 15.738 | 0.983 | 1.00 | 26.53 | H | C |
| ATOM | 837 | NE1 | TRP | H | 103 | 10.442 | 15.036 | 0.004 | 1.00 | 23.88 | H | N |
| ATOM | 838 | CZ2 | TRP | H | 103 | 9.712 | 13.719 | −2.009 | 1.00 | 28.87 | H | C |
| ATOM | 839 | CZ3 | TRP | H | 103 | 7.300 | 13.685 | −2.274 | 1.00 | 21.44 | H | C |
| ATOM | 840 | CH2 | TRP | H | 103 | 8.587 | 13.317 | −2.696 | 1.00 | 31.95 | H | C |
| ATOM | 841 | C | TRP | H | 103 | 6.100 | 18.162 | 2.542 | 1.00 | 22.51 | H | C |
| ATOM | 842 | O | TRP | H | 103 | 6.273 | 18.415 | 3.728 | 1.00 | 29.08 | H | O |
| ATOM | 843 | N | GLY | H | 104 | 4.902 | 18.168 | 1.959 | 1.00 | 25.10 | H | N |
| ATOM | 844 | CA | GLY | H | 104 | 3.700 | 18.407 | 2.749 | 1.00 | 20.27 | H | C |
| ATOM | 845 | C | GLY | H | 104 | 3.277 | 17.068 | 3.366 | 1.00 | 20.58 | H | C |
| ATOM | 846 | O | GLY | H | 104 | 3.904 | 16.032 | 3.121 | 1.00 | 15.95 | H | O |
| ATOM | 847 | N | GLN | H | 105 | 2.200 | 17.045 | 4.141 | 1.00 | 21.55 | H | N |
| ATOM | 848 | CA | GLN | H | 105 | 1.790 | 15.789 | 4.754 | 1.00 | 20.11 | H | C |
| ATOM | 849 | CB | GLN | H | 105 | 0.832 | 16.044 | 5.908 | 1.00 | 23.24 | H | C |
| ATOM | 850 | CG | GLN | H | 105 | −0.608 | 16.287 | 5.488 | 1.00 | 23.62 | H | C |
| ATOM | 851 | CD | GLN | H | 105 | −0.864 | 17.701 | 5.035 | 1.00 | 22.81 | H | C |
| ATOM | 852 | OE1 | GLN | H | 105 | 0.059 | 18.505 | 4.905 | 1.00 | 25.95 | H | O |
| ATOM | 853 | NE2 | GLN | H | 105 | −2.130 | 18.027 | 4.828 | 1.00 | 21.91 | H | N |
| ATOM | 854 | C | GLN | H | 105 | 1.152 | 14.816 | 3.758 | 1.00 | 24.47 | H | C |
| ATOM | 855 | O | GLN | H | 105 | 1.223 | 13.609 | 3.930 | 1.00 | 23.93 | H | O |
| ATOM | 856 | N | GLY | H | 106 | 0.544 | 15.352 | 2.709 | 1.00 | 24.25 | H | N |
| ATOM | 857 | CA | GLY | H | 106 | −0.093 | 14.511 | 1.713 | 1.00 | 33.53 | H | C |
| ATOM | 858 | C | GLY | H | 106 | −1.612 | 14.553 | 1.781 | 1.00 | 30.26 | H | C |
| ATOM | 859 | O | GLY | H | 106 | −2.198 | 14.493 | 2.859 | 1.00 | 34.41 | H | O |
| ATOM | 860 | N | THR | H | 107 | −2.248 | 14.752 | 0.637 | 1.00 | 28.46 | H | N |
| ATOM | 861 | CA | THR | H | 107 | −3.707 | 14.785 | 0.585 | 1.00 | 29.64 | H | C |
| ATOM | 862 | CB | THR | H | 107 | −4.243 | 16.171 | 0.085 | 1.00 | 24.04 | H | C |
| ATOM | 863 | OG1 | THR | H | 107 | −5.603 | 16.341 | 0.492 | 1.00 | 30.40 | H | O |
| ATOM | 864 | CG2 | THR | H | 107 | −4.187 | 16.285 | −1.412 | 1.00 | 29.68 | H | C |
| ATOM | 865 | C | THR | H | 107 | −4.167 | 13.621 | −0.300 | 1.00 | 25.01 | H | C |
| ATOM | 866 | O | THR | H | 107 | −3.601 | 13.377 | −1.369 | 1.00 | 22.65 | H | O |
| ATOM | 867 | N | LEU | H | 108 | −5.095 | 12.829 | 0.223 | 1.00 | 25.10 | H | N |
| ATOM | 868 | CA | LEU | H | 108 | −5.632 | 11.676 | −0.498 | 1.00 | 26.38 | H | C |
| ATOM | 869 | CB | LEU | H | 108 | −6.085 | 10.595 | 0.488 | 1.00 | 20.59 | H | C |
| ATOM | 870 | CG | LEU | H | 108 | −6.923 | 9.445 | −0.086 | 1.00 | 26.76 | H | C |
| ATOM | 871 | CD1 | LEU | H | 108 | −6.127 | 8.601 | −1.077 | 1.00 | 17.51 | H | C |
| ATOM | 872 | CD2 | LEU | H | 108 | −7.451 | 8.584 | 1.033 | 1.00 | 15.91 | H | C |
| ATOM | 873 | C | LEU | H | 108 | −6.811 | 12.064 | −1.381 | 1.00 | 26.17 | H | C |
| ATOM | 874 | O | LEU | H | 108 | −7.751 | 12.689 | −0.903 | 1.00 | 26.42 | H | O |
| ATOM | 875 | N | VAL | H | 109 | −6.719 | 11.736 | −2.673 | 1.00 | 26.36 | H | N |
| ATOM | 876 | CA | VAL | H | 109 | −7.800 | 11.986 | −3.619 | 1.00 | 26.09 | H | C |
| ATOM | 877 | CB | VAL | H | 109 | −7.338 | 12.799 | −4.838 | 1.00 | 23.68 | H | C |
| ATOM | 878 | CG1 | VAL | H | 109 | −8.411 | 12.777 | −5.917 | 1.00 | 21.47 | H | C |
| ATOM | 879 | CG2 | VAL | H | 109 | −7.076 | 14.235 | −4.446 | 1.00 | 24.28 | H | C |
| ATOM | 880 | C | VAL | H | 109 | −8.314 | 10.615 | −4.076 | 1.00 | 28.04 | H | C |
| ATOM | 881 | O | VAL | H | 109 | −7.563 | 9.802 | −4.608 | 1.00 | 32.49 | H | O |
| ATOM | 882 | N | THR | H | 110 | −9.578 | 10.340 | −3.790 | 1.00 | 30.71 | H | N |
| ATOM | 883 | CA | THR | H | 110 | −10.205 | 9.075 | −4.156 | 1.00 | 32.43 | H | C |
| ATOM | 884 | CB | THR | H | 110 | −10.998 | 8.514 | −2.964 | 1.00 | 31.56 | H | C |
| ATOM | 885 | OG1 | THR | H | 110 | −10.111 | 8.339 | −1.856 | 1.00 | 39.39 | H | O |
| ATOM | 886 | CG2 | THR | H | 110 | −11.609 | 7.175 | −3.297 | 1.00 | 36.86 | H | C |
| ATOM | 887 | C | THR | H | 110 | −11.148 | 9.334 | −5.323 | 1.00 | 31.61 | H | C |
| ATOM | 888 | O | THR | H | 110 | −12.008 | 10.212 | −5.235 | 1.00 | 30.05 | H | O |
| ATOM | 889 | N | VAL | H | 111 | −10.933 | 8.629 | −6.433 | 1.00 | 26.60 | H | N |
| ATOM | 890 | CA | VAL | H | 111 | −11.775 | 8.778 | −7.620 | 1.00 | 30.20 | H | C |
| ATOM | 891 | CB | VAL | H | 111 | −10.925 | 8.891 | −8.884 | 1.00 | 28.59 | H | C |
| ATOM | 892 | CG1 | VAL | H | 111 | −11.824 | 8.927 | −10.108 | 1.00 | 27.11 | H | C |
| ATOM | 893 | CG2 | VAL | H | 111 | −10.057 | 10.137 | −8.816 | 1.00 | 27.19 | H | C |
| ATOM | 894 | C | VAL | H | 111 | −12.684 | 7.563 | −7.748 | 1.00 | 29.58 | H | C |
| ATOM | 895 | O | VAL | H | 111 | −12.220 | 6.477 | −8.102 | 1.00 | 37.97 | H | O |
| ATOM | 896 | N | SER | H | 112 | −13.982 | 7.756 | −7.534 | 1.00 | 27.20 | H | N |
| ATOM | 897 | CA | SER | H | 112 | −14.920 | 6.632 | −7.576 | 1.00 | 30.49 | H | C |
| ATOM | 898 | CB | SER | H | 112 | −14.715 | 5.789 | −6.306 | 1.00 | 27.84 | H | C |
| ATOM | 899 | OG | SER | H | 112 | −15.687 | 4.772 | −6.152 | 1.00 | 39.41 | H | O |
| ATOM | 900 | C | SER | H | 112 | −16.377 | 7.074 | −7.670 | 1.00 | 28.47 | H | C |
| ATOM | 901 | O | SER | H | 112 | −16.738 | 8.146 | −7.207 | 1.00 | 31.53 | H | O |
| ATOM | 902 | N | SER | H | 113 | −17.213 | 6.246 | −8.278 | 1.00 | 30.70 | H | N |
| ATOM | 903 | CA | SER | H | 113 | −18.632 | 6.565 | −8.399 | 1.00 | 30.01 | H | C |
| ATOM | 904 | CB | SER | H | 113 | −19.148 | 6.281 | −9.826 | 1.00 | 34.63 | H | C |
| ATOM | 905 | OG | SER | H | 113 | −19.019 | 4.915 | −10.195 | 1.00 | 27.18 | H | O |
| ATOM | 906 | C | SER | H | 113 | −19.446 | 5.805 | −7.345 | 1.00 | 26.77 | H | C |
| ATOM | 907 | O | SER | H | 113 | −20.677 | 5.873 | −7.325 | 1.00 | 33.34 | H | O |
| ATOM | 908 | N | ALA | H | 114 | −18.744 | 5.119 | −6.446 | 1.00 | 24.66 | H | N |
| ATOM | 909 | CA | ALA | H | 114 | −19.374 | 4.365 | −5.355 | 1.00 | 21.45 | H | C |
| ATOM | 910 | CB | ALA | H | 114 | −18.363 | 3.507 | −4.649 | 1.00 | 15.84 | H | C |
| ATOM | 911 | C | ALA | H | 114 | −20.055 | 5.263 | −4.334 | 1.00 | 26.90 | H | C |
| ATOM | 912 | O | ALA | H | 114 | −19.652 | 6.421 | −4.099 | 1.00 | 26.15 | H | O |
| ATOM | 913 | N | SER | H | 115 | −21.073 | 4.693 | −3.701 | 1.00 | 29.70 | H | N |
| ATOM | 914 | CA | SER | H | 115 | −21.856 | 5.381 | −2.692 | 1.00 | 25.59 | H | C |
| ATOM | 915 | CB | SER | H | 115 | −23.352 | 5.140 | −2.965 | 1.00 | 25.47 | H | C |
| ATOM | 916 | OG | SER | H | 115 | −23.668 | 5.536 | −4.291 | 1.00 | 34.27 | H | O |
| ATOM | 917 | C | SER | H | 115 | −21.469 | 4.830 | −1.328 | 1.00 | 24.44 | H | C |
| ATOM | 918 | O | SER | H | 115 | −21.063 | 3.668 | −1.219 | 1.00 | 16.03 | H | O |
| ATOM | 919 | N | THR | H | 116 | −21.593 | 5.668 | −0.297 | 1.00 | 26.95 | H | N |
| ATOM | 920 | CA | THR | H | 116 | −21.280 | 5.279 | 1.075 | 1.00 | 23.95 | H | C |
| ATOM | 921 | CB | THR | H | 116 | −21.627 | 6.401 | 2.042 | 1.00 | 29.55 | H | C |
| ATOM | 922 | OG1 | THR | H | 116 | −20.960 | 7.595 | 1.632 | 1.00 | 24.12 | H | O |
| ATOM | 923 | CG2 | THR | H | 116 | −21.218 | 6.035 | 3.458 | 1.00 | 25.30 | H | C |
| ATOM | 924 | C | THR | H | 116 | −22.128 | 4.076 | 1.452 | 1.00 | 26.42 | H | C |
| ATOM | 925 | O | THR | H | 116 | −23.338 | 4.063 | 1.222 | 1.00 | 30.46 | H | O |
| ATOM | 926 | N | LYS | H | 117 | −21.480 | 3.066 | 2.014 | 1.00 | 28.22 | H | N |
| ATOM | 927 | CA | LYS | H | 117 | −22.153 | 1.846 | 2.424 | 1.00 | 27.56 | H | C |
| ATOM | 928 | CB | LYS | H | 117 | −22.260 | 0.884 | 1.233 | 1.00 | 26.70 | H | C |
| ATOM | 929 | CG | LYS | H | 117 | −22.913 | −0.457 | 1.566 | 1.00 | 25.19 | H | C |
| ATOM | 930 | CD | LYS | H | 117 | −22.842 | −1.383 | 0.362 | 1.00 | 28.36 | H | C |
| ATOM | 931 | CE | LYS | H | 117 | −23.446 | −2.758 | 0.650 | 1.00 | 35.03 | H | C |
| ATOM | 932 | NZ | LYS | H | 117 | −22.711 | −3.531 | 1.705 | 1.00 | 32.59 | H | N |
| ATOM | 933 | C | LYS | H | 117 | −21.406 | 1.171 | 3.580 | 1.00 | 29.56 | H | C |
| ATOM | 934 | O | LYS | H | 117 | −20.188 | 0.999 | 3.531 | 1.00 | 30.21 | H | O |
| ATOM | 935 | N | GLY | H | 118 | −22.150 | 0.825 | 4.626 | 1.00 | 31.93 | H | N |
| ATOM | 936 | CA | GLY | H | 118 | −21.577 | 0.152 | 5.774 | 1.00 | 31.31 | H | C |
| ATOM | 937 | C | GLY | H | 118 | −21.330 | −1.294 | 5.414 | 1.00 | 34.22 | H | C |
| ATOM | 938 | O | GLY | H | 118 | −22.001 | −1.830 | 4.530 | 1.00 | 40.61 | H | O |
| ATOM | 939 | N | PRO | H | 119 | −20.396 | −1.970 | 6.093 | 1.00 | 29.50 | H | N |
| ATOM | 940 | CD | PRO | H | 119 | −19.458 | −1.479 | 7.112 | 1.00 | 26.47 | H | C |
| ATOM | 941 | CA | PRO | H | 119 | −20.116 | −3.365 | 5.771 | 1.00 | 27.83 | H | C |
| ATOM | 942 | CB | PRO | H | 119 | −18.725 | −3.552 | 6.345 | 1.00 | 28.01 | H | C |
| ATOM | 943 | CG | PRO | H | 119 | −18.815 | −2.764 | 7.607 | 1.00 | 22.59 | H | C |
| ATOM | 944 | C | PRO | H | 119 | −21.050 | −4.380 | 6.396 | 1.00 | 30.00 | H | C |
| ATOM | 945 | O | PRO | H | 119 | −21.805 | −4.074 | 7.312 | 1.00 | 34.45 | H | O |
| ATOM | 946 | N | SER | H | 120 | −20.988 | −5.588 | 5.848 | 1.00 | 29.08 | H | N |
| ATOM | 947 | CA | SER | H | 120 | −21.715 | −6.732 | 6.353 | 1.00 | 30.79 | H | C |
| ATOM | 948 | CB | SER | H | 120 | −22.224 | −7.610 | 5.202 | 1.00 | 31.44 | H | C |
| ATOM | 949 | OG | SER | H | 120 | −23.077 | −6.891 | 4.329 | 1.00 | 44.03 | H | O |
| ATOM | 950 | C | SER | H | 120 | −20.567 | −7.446 | 7.078 | 1.00 | 28.70 | H | C |
| ATOM | 951 | O | SER | H | 120 | −19.447 | −7.508 | 6.559 | 1.00 | 31.24 | H | O |
| ATOM | 952 | N | VAL | H | 121 | −20.799 | −7.916 | 8.295 | 1.00 | 27.06 | H | N |
| ATOM | 953 | CA | VAL | H | 121 | −19.732 | −8.602 | 9.016 | 1.00 | 27.82 | H | C |
| ATOM | 954 | CB | VAL | H | 121 | −19.480 | −7.988 | 10.409 | 1.00 | 32.13 | H | C |
| ATOM | 955 | CG1 | VAL | H | 121 | −18.271 | −8.664 | 11.085 | 1.00 | 30.82 | H | C |
| ATOM | 956 | CG2 | VAL | H | 121 | −19.253 | −6.495 | 10.284 | 1.00 | 29.46 | H | C |
| ATOM | 957 | C | VAL | H | 121 | −20.085 | −10.052 | 9.183 | 1.00 | 26.87 | H | C |
| ATOM | 958 | O | VAL | H | 121 | −21.010 | −10.372 | 9.914 | 1.00 | 30.18 | H | O |
| ATOM | 959 | N | PHE | H | 122 | −19.330 | −10.928 | 8.524 | 1.00 | 28.17 | H | N |
| ATOM | 960 | CA | PHE | H | 122 | −19.587 | −12.366 | 8.602 | 1.00 | 30.18 | H | C |
| ATOM | 961 | CB | PHE | H | 122 | −19.673 | −12.998 | 7.199 | 1.00 | 29.25 | H | C |
| ATOM | 962 | CG | PHE | H | 122 | −20.707 | −12.372 | 6.298 | 1.00 | 29.97 | H | C |
| ATOM | 963 | CD1 | PHE | H | 122 | −22.060 | −12.423 | 6.619 | 1.00 | 29.85 | H | C |
| ATOM | 964 | CD2 | PHE | H | 122 | −20.326 | −11.751 | 5.103 | 1.00 | 27.90 | H | C |
| ATOM | 965 | CE1 | PHE | H | 122 | −23.025 | −11.863 | 5.761 | 1.00 | 27.28 | H | C |
| ATOM | 966 | CE2 | PHE | H | 122 | −21.282 | −11.189 | 4.241 | 1.00 | 28.10 | H | C |
| ATOM | 967 | CZ | PHE | H | 122 | −22.635 | −11.248 | 4.572 | 1.00 | 26.11 | H | C |
| ATOM | 968 | C | PHE | H | 122 | −18.501 | −13.072 | 9.395 | 1.00 | 33.15 | H | C |
| ATOM | 969 | O | PHE | H | 122 | −17.328 | −12.708 | 9.316 | 1.00 | 34.33 | H | O |
| ATOM | 970 | N | PRO | H | 123 | −18.875 | −14.119 | 10.151 | 1.00 | 35.67 | H | N |
| ATOM | 971 | CD | PRO | H | 123 | −20.239 | −14.611 | 10.413 | 1.00 | 28.27 | H | C |
| ATOM | 972 | CA | PRO | H | 123 | −17.906 | −14.865 | 10.950 | 1.00 | 31.54 | H | C |
| ATOM | 973 | CB | PRO | H | 123 | −18.795 | −15.576 | 11.958 | 1.00 | 31.87 | H | C |
| ATOM | 974 | CG | PRO | H | 123 | −19.973 | −15.924 | 11.118 | 1.00 | 27.59 | H | C |
| ATOM | 975 | C | PRO | H | 123 | −17.086 | −15.869 | 10.145 | 1.00 | 36.31 | H | C |
| ATOM | 976 | O | PRO | H | 123 | −17.529 | −16.380 | 9.114 | 1.00 | 38.30 | H | O |
| ATOM | 977 | N | LEU | H | 124 | −15.857 | −16.079 | 10.612 | 1.00 | 39.98 | H | N |
| ATOM | 978 | CA | LEU | H | 124 | −14.918 | −17.042 | 10.050 | 1.00 | 41.91 | H | C |
| ATOM | 979 | CB | LEU | H | 124 | −13.606 | −16.356 | 9.656 | 1.00 | 35.57 | H | C |
| ATOM | 980 | CG | LEU | H | 124 | −13.693 | −15.347 | 8.499 | 1.00 | 37.04 | H | C |
| ATOM | 981 | CD1 | LEU | H | 124 | −12.408 | −14.551 | 8.380 | 1.00 | 34.92 | H | C |
| ATOM | 982 | CD2 | LEU | H | 124 | −13.993 | −16.046 | 7.195 | 1.00 | 27.51 | H | C |
| ATOM | 983 | C | LEU | H | 124 | −14.745 | −17.954 | 11.267 | 1.00 | 43.69 | H | C |
| ATOM | 984 | O | LEU | H | 124 | −13.863 | −17.747 | 12.096 | 1.00 | 50.17 | H | O |
| ATOM | 985 | N | ALA | H | 125 | −15.696 | −18.875 | 11.419 | 1.00 | 46.03 | H | N |
| ATOM | 986 | CA | ALA | H | 125 | −15.763 | −19.814 | 12.538 | 1.00 | 42.41 | H | C |
| ATOM | 987 | CB | ALA | H | 125 | −17.136 | −20.477 | 12.554 | 1.00 | 44.51 | H | C |
| ATOM | 988 | C | ALA | H | 125 | −14.690 | −20.883 | 12.568 | 1.00 | 40.32 | H | C |
| ATOM | 989 | O | ALA | H | 125 | −14.344 | −21.438 | 11.523 | 1.00 | 40.12 | H | O |
| ATOM | 990 | N | PRO | H | 126 | −14.219 | −21.248 | 13.780 | 1.00 | 36.80 | H | N |
| ATOM | 991 | CD | PRO | H | 126 | −14.637 | −20.641 | 15.057 | 1.00 | 34.94 | H | C |
| ATOM | 992 | CA | PRO | H | 126 | −13.182 | −22.271 | 13.999 | 1.00 | 42.51 | H | C |
| ATOM | 993 | CB | PRO | H | 126 | −13.080 | −22.342 | 15.520 | 1.00 | 37.11 | H | C |
| ATOM | 994 | CG | PRO | H | 126 | −13.480 | −20.966 | 15.960 | 1.00 | 41.59 | H | C |
| ATOM | 995 | C | PRO | H | 126 | −13.564 | −23.633 | 13.409 | 1.00 | 49.40 | H | C |
| ATOM | 996 | O | PRO | H | 126 | −14.746 | −23.988 | 13.368 | 1.00 | 54.12 | H | O |
| ATOM | 997 | N | SER | H | 127 | −12.554 | −24.386 | 12.966 | 1.00 | 59.27 | H | N |
| ATOM | 998 | CA | SER | H | 127 | −12.723 | −25.715 | 12.353 | 1.00 | 66.53 | H | C |
| ATOM | 999 | CB | SER | H | 127 | −11.373 | −26.233 | 11.823 | 1.00 | 65.34 | H | C |
| ATOM | 1000 | OG | SER | H | 127 | −10.805 | −25.359 | 10.857 | 1.00 | 68.65 | H | O |
| ATOM | 1001 | C | SER | H | 127 | −13.315 | −26.778 | 13.285 | 1.00 | 70.15 | H | C |
| ATOM | 1002 | O | SER | H | 127 | −14.051 | −27.671 | 12.838 | 1.00 | 70.93 | H | O |
| ATOM | 1003 | N | SER | H | 128 | −12.964 | −26.687 | 14.569 | 1.00 | 75.76 | H | N |
| ATOM | 1004 | CA | SER | H | 128 | −43.422 | −27.627 | 15.601 | 1.00 | 80.27 | H | C |
| ATOM | 1005 | CB | SER | H | 128 | −14.952 | −27.562 | 15.785 | 1.00 | 79.58 | H | C |
| ATOM | 1006 | OG | SER | H | 128 | −15.398 | −28.461 | 16.796 | 1.00 | 76.41 | H | O |
| ATOM | 1007 | C | SER | H | 128 | −12.993 | −29.060 | 15.281 | 1.00 | 81.02 | H | C |
| ATOM | 1008 | O | SER | H | 128 | −12.647 | −29.825 | 16.181 | 1.00 | 83.09 | H | O |
| ATOM | 1009 | N | GLY | H | 133 | −3.440 | −29.915 | 16.559 | 1.00 | 68.04 | H | N |
| ATOM | 1010 | CA | GLY | H | 133 | −2.683 | −28.871 | 17.230 | 1.00 | 65.57 | H | C |
| ATOM | 1011 | C | GLY | H | 133 | −3.493 | −28.199 | 18.319 | 1.00 | 64.32 | H | C |
| ATOM | 1012 | O | GLY | H | 133 | −4.719 | −28.340 | 18.357 | 1.00 | 67.09 | H | O |
| ATOM | 1013 | N | GLY | H | 134 | −2.819 | −27.475 | 19.209 | 1.00 | 58.05 | H | N |
| ATOM | 1014 | CA | GLY | H | 134 | −3.527 | −26.798 | 20.282 | 1.00 | 54.06 | H | C |
| ATOM | 1015 | C | GLY | H | 134 | −3.923 | −25.373 | 19.944 | 1.00 | 53.45 | H | C |
| ATOM | 1016 | O | GLY | H | 134 | −4.301 | −24.612 | 20.837 | 1.00 | 52.81 | H | O |
| ATOM | 1017 | N | THR | H | 135 | −3.858 | −25.022 | 18.659 | 1.00 | 51.17 | H | N |
| ATOM | 1018 | CA | THR | H | 135 | −4.190 | −23.677 | 18.207 | 1.00 | 47.50 | H | C |
| ATOM | 1019 | CB | THR | H | 135 | −2.908 | −22.908 | 17.790 | 1.00 | 46.76 | H | C |
| ATOM | 1020 | OG1 | THR | H | 135 | −2.119 | −22.640 | 18.957 | 1.00 | 49.43 | H | O |
| ATOM | 1021 | CG2 | THR | H | 135 | −3.244 | −21.581 | 17.105 | 1.00 | 43.70 | H | C |
| ATOM | 1022 | C | THR | H | 135 | −5.218 | −23.641 | 17.077 | 1.00 | 45.62 | H | C |
| ATOM | 1023 | O | THR | H | 135 | −5.113 | −24.363 | 16.090 | 1.00 | 46.26 | H | O |
| ATOM | 1024 | N | ALA | H | 136 | −6.216 | −22.784 | 17.243 | 1.00 | 43.83 | H | N |
| ATOM | 1025 | CA | ALA | H | 136 | −7.267 | −22.619 | 16.251 | 1.00 | 44.25 | H | C |
| ATOM | 1026 | CB | ALA | H | 136 | −8.596 | −23.052 | 16.830 | 1.00 | 39.06 | H | C |
| ATOM | 1027 | C | ALA | H | 136 | −7.328 | −21.154 | 15.822 | 1.00 | 42.88 | H | C |
| ATOM | 1028 | O | ALA | H | 136 | −6.883 | −20.260 | 16.544 | 1.00 | 46.11 | H | O |
| ATOM | 1029 | N | ALA | H | 137 | −7.835 | −20.918 | 14.620 | 1.00 | 39.58 | H | N |
| ATOM | 1030 | CA | ALA | H | 137 | −7.954 | −19.569 | 14.108 | 1.00 | 36.64 | H | C |
| ATOM | 1031 | CB | ALA | H | 137 | −7.175 | −19.430 | 12.808 | 1.00 | 36.84 | H | C |
| ATOM | 1032 | C | ALA | H | 137 | −9.422 | −19.294 | 13.871 | 1.00 | 35.60 | H | C |
| ATOM | 1033 | O | ALA | H | 137 | −10.180 | −20.188 | 13.518 | 1.00 | 36.12 | H | O |
| ATOM | 1034 | N | LEU | H | 138 | −9.826 | −18.057 | 14.100 | 1.00 | 37.42 | H | N |
| ATOM | 1035 | CA | LEU | H | 138 | −11.205 | −17.642 | 13.894 | 1.00 | 37.91 | H | C |
| ATOM | 1036 | CB | LEU | H | 138 | −12.006 | −17.769 | 15.194 | 1.00 | 40.58 | H | C |
| ATOM | 1037 | CG | LEU | H | 138 | −11.530 | −17.020 | 16.443 | 1.00 | 40.63 | H | C |
| ATOM | 1038 | CD1 | LEU | H | 138 | −11.988 | −15.579 | 16.411 | 1.00 | 46.17 | H | C |
| ATOM | 1039 | CD2 | LEU | H | 138 | −12.092 | −17.690 | 17.670 | 1.00 | 44.63 | H | C |
| ATOM | 1040 | C | LEU | H | 138 | −11.145 | −16.196 | 13.448 | 1.00 | 38.77 | H | C |
| ATOM | 1041 | O | LEU | H | 138 | −10.091 | −15.563 | 13.545 | 1.00 | 40.07 | H | O |
| ATOM | 1042 | N | GLY | H | 139 | −12.264 | −15.661 | 12.980 | 1.00 | 36.72 | H | N |
| ATOM | 1043 | CA | GLY | H | 139 | −12.242 | −14.284 | 12.539 | 1.00 | 37.91 | H | C |
| ATOM | 1044 | C | GLY | H | 139 | −13.517 | −13.719 | 11.966 | 1.00 | 38.10 | H | C |
| ATOM | 1045 | O | GLY | H | 139 | −14.585 | −14.309 | 12.081 | 1.00 | 41.18 | H | O |
| ATOM | 1046 | N | CYS | H | 140 | −13.380 | −12.546 | 11.356 | 1.00 | 39.36 | H | N |
| ATOM | 1047 | CA | CYS | H | 140 | −14.485 | −11.833 | 10.741 | 1.00 | 37.33 | H | C |
| ATOM | 1048 | C | CYS | H | 140 | −14.157 | −11.348 | 9.340 | 1.00 | 32.87 | H | C |
| ATOM | 1049 | O | CYS | H | 140 | −13.051 | −10.903 | 9.060 | 1.00 | 30.77 | H | O |
| ATOM | 1050 | CB | CYS | H | 140 | −14.874 | −10.618 | 11.586 | 1.00 | 38.67 | H | C |
| ATOM | 1051 | SG | CYS | H | 140 | −15.768 | −11.006 | 13.120 | 1.00 | 51.11 | H | S |
| ATOM | 1052 | N | LEU | H | 141 | −15.150 | −11.438 | 8.470 | 1.00 | 32.70 | H | N |
| ATOM | 1053 | CA | LEU | H | 141 | −15.046 | −10.968 | 7.104 | 1.00 | 28.80 | H | C |
| ATOM | 1054 | CB | LEU | H | 141 | −15.652 | −11.999 | 6.150 | 1.00 | 25.09 | H | C |
| ATOM | 1055 | CG | LEU | H | 141 | −15.780 | −11.596 | 4.683 | 1.00 | 28.67 | H | C |
| ATOM | 1056 | CD1 | LEU | H | 141 | −14.400 | −11.392 | 4.116 | 1.00 | 25.57 | H | C |
| ATOM | 1057 | CD2 | LEU | H | 141 | −16.525 | −12.668 | 3.883 | 1.00 | 24.48 | H | C |
| ATOM | 1058 | C | LEU | H | 141 | −15.860 | −9.674 | 7.077 | 1.00 | 24.78 | H | C |
| ATOM | 1059 | O | LEU | H | 141 | −17.071 | −9.694 | 7.260 | 1.00 | 34.11 | H | O |
| ATOM | 1060 | N | VAL | H | 142 | −15.174 | −8.547 | 6.955 | 1.00 | 27.91 | H | N |
| ATOM | 1061 | CA | VAL | H | 142 | −15.808 | −7.237 | 6.897 | 1.00 | 20.17 | H | C |
| ATOM | 1062 | CB | VAL | H | 142 | −14.954 | −6.230 | 7.656 | 1.00 | 21.99 | H | C |
| ATOM | 1063 | CG1 | VAL | H | 142 | −15.620 | −4.864 | 7.730 | 1.00 | 15.98 | H | C |
| ATOM | 1064 | CG2 | VAL | H | 142 | −14.710 | −6.774 | 9.058 | 1.00 | 16.36 | H | C |
| ATOM | 1065 | C | VAL | H | 142 | −15.905 | −6.922 | 5.406 | 1.00 | 27.82 | H | C |
| ATOM | 1066 | O | VAL | H | 142 | −14.980 | −6.401 | 4.800 | 1.00 | 28.85 | H | O |
| ATOM | 1067 | N | LYS | H | 143 | −17.060 | −7.249 | 4.835 | 1.00 | 28.26 | H | N |
| ATOM | 1068 | CA | LYS | H | 143 | −17.303 | −7.118 | 3.411 | 1.00 | 25.66 | H | C |
| ATOM | 1069 | CB | LYS | H | 143 | −17.778 | −8.489 | 2.899 | 1.00 | 29.03 | H | C |
| ATOM | 1070 | CG | LYS | H | 143 | −17.964 | −8.612 | 1.399 | 1.00 | 28.57 | H | C |
| ATOM | 1071 | CD | LYS | H | 143 | −18.443 | −10.012 | 0.990 | 1.00 | 37.13 | H | C |
| ATOM | 1072 | CE | LYS | H | 143 | −18.665 | −10.132 | −0.527 | 1.00 | 37.73 | H | C |
| ATOM | 1073 | HZ | LYS | H | 143 | −17.435 | −9.857 | −1.346 | 1.00 | 37.43 | H | N |
| ATOM | 1074 | C | LYS | H | 143 | −18.239 | −6.031 | 2.882 | 1.00 | 29.12 | H | C |
| ATOM | 1075 | O | LYS | H | 143 | −19.253 | −5.687 | 3.499 | 1.00 | 26.85 | H | O |
| ATOM | 1076 | N | ASP | H | 144 | −17.885 | −5.548 | 1.690 | 1.00 | 29.90 | H | N |
| ATOM | 1077 | CA | ASP | H | 144 | −18.624 | −4.548 | 0.919 | 1.00 | 29.85 | H | C |
| ATOM | 1078 | CB | ASP | H | 144 | −19.932 | −5.154 | 0.392 | 1.00 | 29.70 | H | C |
| ATOM | 1079 | CG | ASP | H | 144 | −19.697 | −6.369 | −0.501 | 1.00 | 35.04 | H | C |
| ATOM | 1080 | OD1 | ASP | H | 144 | −18.650 | −6.400 | −1.195 | 1.00 | 32.75 | H | O |
| ATOM | 1081 | OD2 | ASP | H | 144 | −20.555 | −7.289 | −0.506 | 1.00 | 34.10 | H | O |
| ATOM | 1082 | C | ASP | H | 144 | −18.890 | −3.199 | 1.555 | 1.00 | 26.62 | H | C |
| ATOM | 1083 | O | ASP | H | 144 | −20.037 | −2.825 | 1.772 | 1.00 | 30.10 | H | O |
| ATOM | 1084 | N | TYR | H | 145 | −17.829 | −2.459 | 1.834 | 1.00 | 22.63 | H | N |
| ATOM | 1085 | CA | TYR | H | 145 | −17.992 | −1.143 | 2.425 | 1.00 | 22.01 | H | C |
| ATOM | 1086 | CB | TYR | H | 145 | −17.484 | −1.091 | 3.885 | 1.00 | 25.82 | H | C |
| ATOM | 1087 | CG | TYR | H | 145 | −15.989 | −1.296 | 4.071 | 1.00 | 23.49 | H | C |
| ATOM | 1088 | CD1 | TYR | H | 145 | −15.447 | −2.579 | 4.140 | 1.00 | 25.53 | H | C |
| ATOM | 1089 | CE1 | TYR | H | 145 | −14.068 | −2.781 | 4.293 | 1.00 | 30.11 | H | C |
| ATOM | 1090 | CD2 | TYR | H | 145 | −15.120 | −0.210 | 4.165 | 1.00 | 23.12 | H | C |
| ATOM | 1091 | CE2 | TYR | H | 145 | −13.732 | −0.399 | 4.318 | 1.00 | 28.20 | H | C |
| ATOM | 1092 | CZ | TYR | H | 145 | −13.218 | −1.693 | 4.380 | 1.00 | 27.44 | H | C |
| ATOM | 1093 | OH | TYR | H | 145 | −11.869 | −1.913 | 4.530 | 1.00 | 26.79 | H | O |
| ATOM | 1094 | C | TYR | H | 145 | −17.284 | −0.097 | 1.598 | 1.00 | 23.72 | H | C |
| ATOM | 1095 | O | TYR | H | 145 | −16.386 | −0.409 | 0.816 | 1.00 | 21.97 | H | O |
| ATOM | 1096 | N | PHE | H | 146 | −17.730 | 1.142 | 1.753 | 1.00 | 19.65 | H | N |
| ATOM | 1097 | CA | PHE | H | 146 | −17.144 | 2.263 | 1.066 | 1.00 | 17.46 | H | C |
| ATOM | 1098 | CB | PHE | H | 146 | −17.594 | 2.322 | −0.387 | 1.00 | 17.76 | H | C |
| ATOM | 1099 | CG | PHE | H | 146 | −17.013 | 3.484 | −1.159 | 1.00 | 25.17 | H | C |
| ATOM | 1100 | CD1 | PHE | H | 146 | −17.635 | 4.740 | −1.135 | 1.00 | 24.10 | H | C |
| ATOM | 1101 | CD2 | PHE | H | 146 | −15.840 | 3.329 | −1.909 | 1.00 | 21.88 | H | C |
| ATOM | 1102 | CE1 | PHE | H | 146 | −17.095 | 5.825 | −1.844 | 1.00 | 25.81 | H | C |
| ATOM | 1103 | CE2 | PHE | H | 146 | −15.290 | 4.400 | −2.618 | 1.00 | 19.86 | H | C |
| ATOM | 1104 | CZ | PHE | H | 146 | −15.911 | 5.647 | −2.590 | 1.00 | 28.69 | H | C |
| ATOM | 1105 | C | PHE | H | 146 | −17.594 | 3.491 | 1.819 | 1.00 | 20.61 | H | C |
| ATOM | 1106 | O | PHE | H | 146 | −18.740 | 3.572 | 2.234 | 1.00 | 22.36 | H | O |
| ATOM | 1107 | N | PRO | H | 147 | −16.663 | 4.425 | 2.080 | 1.00 | 27.27 | H | N |
| ATOM | 1108 | CD | PRO | H | 147 | −16.968 | 5.750 | 2.664 | 1.00 | 27.74 | H | C |
| ATOM | 1109 | CA | PRO | H | 147 | −15.243 | 4.341 | 1.692 | 1.00 | 26.35 | H | C |
| ATOM | 1110 | CB | PRO | H | 147 | −14.887 | 5.806 | 1.454 | 1.00 | 27.26 | H | C |
| ATOM | 1111 | CG | PRO | H | 147 | −15.600 | 6.478 | 2.617 | 1.00 | 29.00 | H | C |
| ATOM | 1112 | C | PRO | H | 147 | −14.410 | 3.800 | 2.843 | 1.00 | 22.92 | H | C |
| ATOM | 1113 | O | PRO | H | 147 | −14.946 | 3.325 | 3.846 | 1.00 | 30.57 | H | O |
| ATOM | 1114 | N | GLU | H | 148 | −13.096 | 3.862 | 2.690 | 1.00 | 27.18 | H | N |
| ATOM | 1115 | CA | GLU | H | 148 | −12.200 | 3.448 | 3.758 | 1.00 | 29.20 | H | C |
| ATOM | 1116 | CB | GLU | H | 148 | −10.787 | 3.272 | 3.215 | 1.00 | 30.88 | H | C |
| ATOM | 1117 | CG | GLU | H | 148 | −10.652 | 2.127 | 2.237 | 1.00 | 31.56 | H | C |
| ATOM | 1118 | CD | GLU | H | 148 | −9.229 | 1.655 | 2.117 | 1.00 | 35.53 | H | C |
| ATOM | 1119 | OE1 | GLU | H | 148 | −8.541 | 2.091 | 1.168 | 1.00 | 39.60 | H | O |
| ATOM | 1120 | OE2 | GLU | H | 148 | −8.795 | 0.856 | 2.978 | 1.00 | 34.47 | H | O |
| ATOM | 1121 | C | GLU | H | 148 | −12.239 | 4.572 | 4.809 | 1.00 | 29.42 | H | C |
| ATOM | 1122 | O | GLU | H | 148 | −12.457 | 5.742 | 4.465 | 1.00 | 29.69 | H | O |
| ATOM | 1123 | N | PRO | H | 149 | −11.919 | 4.261 | 6.077 | 1.00 | 26.14 | H | N |
| ATOM | 1124 | CD | PRO | H | 149 | −11.679 | 5.313 | 7.089 | 1.00 | 31.23 | H | C |
| ATOM | 1125 | CA | PRO | H | 149 | −11.532 | 2.952 | 6.602 | 1.00 | 27.17 | H | C |
| ATOM | 1126 | CB | PRO | H | 149 | −10.365 | 3.310 | 7.503 | 1.00 | 27.48 | H | C |
| ATOM | 1127 | CG | PRO | H | 149 | −10.919 | 4.557 | 8.219 | 1.00 | 28.88 | H | C |
| ATOM | 1128 | C | PRO | H | 149 | −12.594 | 2.290 | 7.449 | 1.00 | 32.27 | H | C |
| ATOM | 1129 | O | PRO | H | 149 | −13.689 | 2.817 | 7.642 | 1.00 | 38.02 | H | O |
| ATOM | 1130 | N | VAL | H | 150 | −12.208 | 1.155 | 8.013 | 1.00 | 35.88 | H | N |
| ATOM | 1131 | CA | VAL | H | 150 | −13.058 | 0.387 | 8.893 | 1.00 | 39.49 | H | C |
| ATOM | 1132 | CB | VAL | H | 150 | −13.712 | −0.809 | 8.136 | 1.00 | 37.30 | H | C |
| ATOM | 1133 | CG1 | VAL | H | 150 | −12.679 | −1.849 | 7.754 | 1.00 | 40.08 | H | C |
| ATOM | 1134 | CG2 | VAL | H | 150 | −14.802 | −1.428 | 8.965 | 1.00 | 41.72 | H | C |
| ATOM | 1135 | C | VAL | H | 150 | −12.124 | −0.080 | 10.012 | 1.00 | 39.78 | H | C |
| ATOM | 1136 | O | VAL | H | 150 | −10.974 | −0.416 | 9.751 | 1.00 | 44.20 | H | O |
| ATOM | 1137 | N | THR | H | 151 | −12.558 | 0.033 | 11.263 | 1.00 | 40.89 | H | N |
| ATOM | 1138 | CA | THR | H | 151 | −11.723 | −0.417 | 12.376 | 1.00 | 37.06 | H | C |
| ATOM | 1139 | CB | THR | H | 151 | −11.666 | 0.597 | 13.558 | 1.00 | 35.43 | H | C |
| ATOM | 1140 | OG1 | THR | H | 151 | −12.988 | 1.013 | 13.922 | 1.00 | 41.63 | H | O |
| ATOM | 1141 | CG2 | THR | H | 151 | −10.825 | 1.805 | 13.195 | 1.00 | 39.52 | H | C |
| ATOM | 1142 | C | THR | H | 151 | −12.219 | −1.750 | 12.907 | 1.00 | 38.76 | H | C |
| ATOM | 1143 | O | THR | H | 151 | −13.420 | −2.029 | 12.883 | 1.00 | 35.62 | H | O |
| ATOM | 1144 | N | VAL | H | 152 | −11.280 | −2.577 | 13.366 | 1.00 | 37.60 | H | N |
| ATOM | 1145 | CA | VAL | H | 152 | −11.598 | −3.885 | 13.927 | 1.00 | 34.88 | H | C |
| ATOM | 1146 | CB | VAL | H | 152 | −11.258 | −5.024 | 12.949 | 1.00 | 33.18 | H | C |
| ATOM | 1147 | CG1 | VAL | H | 152 | −11.518 | −6.368 | 13.595 | 1.00 | 30.75 | H | C |
| ATOM | 1148 | CG2 | VAL | H | 152 | −12.077 | −4.895 | 11.698 | 1.00 | 33.27 | H | C |
| ATOM | 1149 | C | VAL | H | 152 | −10.846 | −4.123 | 15.238 | 1.00 | 37.70 | H | C |
| ATOM | 1150 | O | VAL | H | 152 | −9.640 | −3.912 | 15.318 | 1.00 | 41.36 | H | O |
| ATOM | 1151 | N | SER | H | 153 | −11.582 | −4.541 | 16.266 | 1.00 | 37.98 | H | N |
| ATOM | 1152 | CA | SER | H | 153 | −11.025 | −4.839 | 17.583 | 1.00 | 38.93 | H | C |
| ATOM | 1153 | CB | SER | H | 153 | −11.441 | −3.773 | 18.600 | 1.00 | 42.83 | H | C |
| ATOM | 1154 | OG | SER | H | 153 | −10.990 | −2.482 | 18.224 | 1.00 | 52.11 | H | O |
| ATOM | 1155 | C | SER | H | 153 | −11.639 | −6.161 | 18.000 | 1.00 | 40.94 | H | C |
| ATOM | 1156 | O | SER | H | 153 | −12.641 | −6.585 | 17.426 | 1.00 | 46.92 | H | O |
| ATOM | 1157 | N | TRP | H | 154 | −11.032 | −6.832 | 18.968 | 1.00 | 37.25 | H | N |
| ATOM | 1158 | CA | TRP | H | 154 | −11.571 | −8.099 | 19.449 | 1.00 | 36.56 | H | C |
| ATOM | 1159 | CB | TRP | H | 154 | −10.636 | −9.263 | 19.106 | 1.00 | 31.88 | H | C |
| ATOM | 1160 | CG | TRP | H | 154 | −10.637 | −9.630 | 17.626 | 1.00 | 32.23 | H | C |
| ATOM | 1161 | CD2 | TRP | H | 154 | −11.461 | −10.608 | 16.973 | 1.00 | 25.76 | H | C |
| ATOM | 1162 | CE2 | TRP | H | 154 | −11.089 | −10.628 | 15.617 | 1.00 | 27.74 | H | C |
| ATOM | 1163 | CE3 | TRP | H | 154 | −12.470 | −11.472 | 17.406 | 1.00 | 31.84 | H | C |
| ATOM | 1164 | CD1 | TRP | H | 154 | −9.833 | −9.112 | 16.661 | 1.00 | 28.92 | H | C |
| ATOM | 1165 | NE1 | TRP | H | 154 | −10.094 | −9.704 | 15.455 | 1.00 | 27.73 | H | N |
| ATOM | 1166 | CZ2 | TRP | H | 154 | −11.692 | −11.479 | 14.681 | 1.00 | 31.59 | H | C |
| ATOM | 1167 | CZ3 | TRP | H | 154 | −13.076 | −12.326 | 16.468 | 1.00 | 36.72 | H | C |
| ATOM | 1168 | CH2 | TRP | H | 154 | −12.679 | −12.319 | 15.125 | 1.00 | 33.04 | H | C |
| ATOM | 1169 | C | TRP | H | 154 | −11.816 | −8.007 | 20.953 | 1.00 | 37.94 | H | C |
| ATOM | 1170 | O | TRP | H | 154 | −10.988 | −7.463 | 21.686 | 1.00 | 39.39 | H | O |
| ATOM | 1171 | N | ASN | H | 155 | −12.975 | −8.494 | 21.397 | 1.00 | 38.26 | H | N |
| ATOM | 1172 | CA | ASN | H | 155 | −13.362 | −8.451 | 22.808 | 1.00 | 38.54 | H | C |
| ATOM | 1173 | CB | ASN | H | 155 | −12.535 | −9.431 | 23.626 | 1.00 | 39.27 | H | C |
| ATOM | 1174 | CG | ASN | H | 155 | −12.786 | −10.862 | 23.244 | 1.00 | 40.11 | H | C |
| ATOM | 1175 | OD1 | ASN | H | 155 | −12.023 | −11.755 | 23.627 | 1.00 | 46.52 | H | O |
| ATOM | 1176 | ND2 | ASN | H | 155 | −13.863 | −11.103 | 22.496 | 1.00 | 40.02 | H | N |
| ATOM | 1177 | C | ASN | H | 155 | −13.210 | −7.045 | 23.375 | 1.00 | 42.98 | H | C |
| ATOM | 1178 | O | ASN | H | 155 | −12.500 | −6.824 | 24.370 | 1.00 | 39.02 | H | O |
| ATOM | 1179 | N | SER | H | 156 | −13.826 | −6.092 | 22.678 | 1.00 | 41.46 | H | N |
| ATOM | 1180 | CA | SER | H | 156 | −13.807 | −4.684 | 23.053 | 1.00 | 48.17 | H | C |
| ATOM | 1181 | CB | SER | H | 156 | −14.556 | −4.486 | 24.378 | 1.00 | 45.90 | H | C |
| ATOM | 1182 | OG | SER | H | 156 | −15.139 | −3.194 | 24.456 | 1.00 | 54.06 | H | O |
| ATOM | 1183 | C | SER | H | 156 | −12.398 | −4.078 | 23.111 | 1.00 | 45.78 | H | C |
| ATOM | 1184 | O | SER | H | 156 | −12.247 | −2.869 | 23.305 | 1.00 | 51.92 | H | O |
| ATOM | 1185 | N | GLY | H | 157 | −11.382 | −4.913 | 22.896 | 1.00 | 47.15 | H | N |
| ATOM | 1186 | CA | GLY | H | 157 | −10.000 | −4.458 | 22.929 | 1.00 | 46.27 | H | C |
| ATOM | 1187 | C | GLY | H | 157 | −9.129 | −5.315 | 23.838 | 1.00 | 50.10 | H | C |
| ATOM | 1188 | O | GLY | H | 157 | −7.905 | −5.211 | 23.808 | 1.00 | 49.50 | H | O |
| ATOM | 1189 | N | ALA | H | 158 | −9.758 | −6.193 | 24.617 | 1.00 | 52.96 | H | N |
| ATOM | 1190 | CA | ALA | H | 158 | −9.044 | −7.067 | 25.550 | 1.00 | 52.37 | H | C |
| ATOM | 1191 | CB | ALA | H | 158 | −9.980 | −7.534 | 26.647 | 1.00 | 48.41 | H | C |
| ATOM | 1192 | C | ALA | H | 158 | −8.379 | −8.269 | 24.887 | 1.00 | 55.70 | H | C |
| ATOM | 1193 | O | ALA | H | 158 | −8.028 | −9.243 | 25.560 | 1.00 | 63.25 | H | O |
| ATOM | 1194 | N | LEU | H | 159 | −8.229 | −8.214 | 23.569 | 1.00 | 50.94 | H | N |
| ATOM | 1195 | CA | LEU | H | 159 | −7.597 | −9.296 | 22.831 | 1.00 | 44.22 | H | C |
| ATOM | 1196 | CB | LEU | H | 159 | −8.644 | −10.268 | 22.322 | 1.00 | 38.80 | H | C |
| ATOM | 1197 | CG | LEU | H | 159 | −8.073 | −11.411 | 21.498 | 1.00 | 39.20 | H | C |
| ATOM | 1198 | CD1 | LEU | H | 159 | −7.029 | −12.139 | 22.313 | 1.00 | 42.23 | H | C |
| ATOM | 1199 | CD2 | LEU | H | 159 | −9.182 | −12.359 | 21.066 | 1.00 | 40.41 | H | C |
| ATOM | 1200 | C | LEU | H | 159 | −6.809 | −8.695 | 21.677 | 1.00 | 46.38 | H | C |
| ATOM | 1201 | O | LEU | H | 159 | −7.393 | −8.204 | 20.709 | 1.00 | 44.35 | H | O |
| ATOM | 1202 | N | THR | H | 160 | −5.481 | −8.709 | 21.818 | 1.00 | 44.50 | H | N |
| ATOM | 1203 | CA | THR | H | 160 | −4.567 | −8.147 | 20.828 | 1.00 | 44.83 | H | C |
| ATOM | 1204 | CB | THR | H | 160 | −3.741 | −6.973 | 21.417 | 1.00 | 48.70 | H | C |
| ATOM | 1205 | OG1 | THR | H | 160 | −3.168 | −7.358 | 22.679 | 1.00 | 50.03 | H | O |
| ATOM | 1206 | CG2 | THR | H | 160 | −4.609 | −5.737 | 21.585 | 1.00 | 45.75 | H | C |
| ATOM | 1207 | C | THR | H | 160 | −3.583 | −9.158 | 20.266 | 1.00 | 42.16 | H | C |
| ATOM | 1208 | O | THR | H | 160 | −3.227 | −9.095 | 19.088 | 1.00 | 46.50 | H | O |
| ATOM | 1209 | N | SER | H | 161 | −3.127 | −11.092 | 20.716 | 1.00 | 36.78 | H | C |
| ATOM | 1210 | CA | SER | H | 161 | −2.173 | −11.092 | 20.716 | 1.00 | 36.78 | H | C |
| ATOM | 1211 | CB | SER | H | 161 | −1.694 | −11.865 | 21.942 | 1.00 | 40.52 | H | C |
| ATOM | 1212 | OG | SER | H | 161 | −1.141 | −10.967 | 22.887 | 1.00 | 50.61 | H | O |
| ATOM | 1213 | C | SER | H | 161 | −2.715 | −12.067 | 19.674 | 1.00 | 36.76 | H | C |
| ATOM | 1214 | O | SER | H | 161 | −3.823 | −12.593 | 19.811 | 1.00 | 36.38 | H | O |
| ATOM | 1215 | N | GLY | H | 162 | −1.916 | −12.299 | 18.634 | 1.00 | 28.14 | H | N |
| ATOM | 1216 | CA | GLY | H | 162 | −2.297 | −13.218 | 17.585 | 1.00 | 31.31 | H | C |
| ATOM | 1217 | C | GLY | H | 162 | −3.311 | −12.675 | 16.605 | 1.00 | 31.16 | H | C |
| ATOM | 1218 | O | GLY | H | 162 | −3.785 | −13.404 | 15.740 | 1.00 | 31.92 | H | O |
| ATOM | 1219 | N | VAL | H | 163 | −3.654 | −11.403 | 16.746 | 1.00 | 32.52 | H | N |
| ATOM | 1220 | CA | VAL | H | 163 | −4.617 | −10.774 | 15.862 | 1.00 | 31.19 | H | C |
| ATOM | 1221 | CB | VAL | H | 163 | −5.333 | −9.597 | 16.568 | 1.00 | 34.98 | H | C |
| ATOM | 1222 | CG1 | VAL | H | 163 | −6.352 | −8.969 | 15.631 | 1.00 | 32.69 | H | C |
| ATOM | 1223 | CG2 | VAL | H | 163 | −5.979 | −10.043 | 17.881 | 1.00 | 29.89 | H | C |
| ATOM | 1224 | C | VAL | H | 163 | −3.951 | −10.202 | 14.600 | 1.00 | 36.79 | H | C |
| ATOM | 1225 | O | VAL | H | 163 | −2.909 | −9.522 | 14.685 | 1.00 | 34.00 | H | O |
| ATOM | 1226 | N | HIS | H | 164 | −4.549 | −10.493 | 13.440 | 1.00 | 26.79 | H | N |
| ATOM | 1227 | CA | HIS | H | 164 | −4.079 | −9.959 | 12.163 | 1.00 | 25.91 | H | C |
| ATOM | 1228 | CB | HIS | H | 164 | −3.473 | −11.025 | 11.250 | 1.00 | 26.56 | H | C |
| ATOM | 1229 | CG | HIS | H | 164 | −2.189 | −11.585 | 11.754 | 1.00 | 27.87 | H | C |
| ATOM | 1230 | CD2 | HIS | H | 164 | −1.088 | −10.975 | 12.252 | 1.00 | 23.89 | H | C |
| ATOM | 1231 | ND1 | HIS | H | 164 | −1.962 | −12.940 | 11.853 | 1.00 | 26.88 | H | N |
| ATOM | 1232 | CE1 | HIS | H | 164 | −0.777 | −13.142 | 12.405 | 1.00 | 26.45 | H | C |
| ATOM | 1233 | NE2 | HIS | H | 164 | −0.228 | −11.967 | 12.655 | 1.00 | 25.98 | H | N |
| ATOM | 1234 | C | HIS | H | 164 | −5.242 | −9.341 | 11.430 | 1.00 | 24.51 | H | C |
| ATOM | 1235 | O | HIS | H | 164 | −6.171 | −10.045 | 11.037 | 1.00 | 30.72 | H | O |
| ATOM | 1236 | N | THR | H | 165 | −5.202 | −8.025 | 11.274 | 1.00 | 23.05 | H | N |
| ATOM | 1237 | CA | THR | H | 165 | −6.233 | −7.304 | 10.554 | 1.00 | 23.20 | H | C |
| ATOM | 1238 | CB | THR | H | 165 | −6.711 | −6.071 | 11.358 | 1.00 | 26.48 | H | C |
| ATOM | 1239 | OG1 | THR | H | 165 | −7.419 | −6.522 | 12.518 | 1.00 | 25.77 | H | O |
| ATOM | 1240 | CG2 | THR | H | 165 | −7.628 | −5.192 | 10.528 | 1.00 | 19.60 | H | C |
| ATOM | 1241 | C | THR | H | 165 | −5.572 | −6.913 | 9.233 | 1.00 | 26.93 | H | C |
| ATOM | 1242 | O | THR | H | 165 | −4.731 | −6.006 | 9.185 | 1.00 | 28.13 | H | O |
| ATOM | 1243 | N | PHE | H | 166 | −5.928 | −7.650 | 8.182 | 1.00 | 23.50 | H | N |
| ATOM | 1244 | CA | PHE | H | 166 | −5.383 | −7.470 | 6.834 | 1.00 | 25.21 | H | C |
| ATOM | 1245 | CB | PHE | H | 166 | −5.808 | −8.639 | 5.944 | 1.00 | 25.28 | H | C |
| ATOM | 1246 | CG | PHE | H | 166 | −5.257 | −9.956 | 6.383 | 1.00 | 28.81 | H | C |
| ATOM | 1247 | CD1 | PHE | H | 166 | −3.945 | −10.313 | 6.067 | 1.00 | 28.22 | H | C |
| ATOM | 1248 | CD2 | PHE | H | 166 | −6.030 | −10.827 | 7.141 | 1.00 | 27.39 | H | C |
| ATOM | 1249 | CE1 | PHE | H | 166 | −3.411 | −11.517 | 6.501 | 1.00 | 31.64 | H | C |
| ATOM | 1250 | CE2 | PHE | H | 166 | −5.507 | −12.040 | 7.587 | 1.00 | 32.06 | H | C |
| ATOM | 1251 | CZ | PHE | H | 166 | −4.192 | −12.387 | 7.267 | 1.00 | 34.06 | H | C |
| ATOM | 1252 | C | PHE | H | 166 | −5.751 | −6.181 | 6.128 | 1.00 | 24.38 | H | C |
| ATOM | 1253 | O | PHE | H | 166 | −6.806 | −5.611 | 6.386 | 1.00 | 29.28 | H | O |
| ATOM | 1254 | N | PRO | H | 167 | −4.864 | −5.686 | 5.242 | 1.00 | 20.74 | H | N |
| ATOM | 1255 | CD | PRO | H | 167 | −3.519 | −6.201 | 4.959 | 1.00 | 19.54 | H | C |
| ATOM | 1256 | CA | PRO | H | 167 | −5.108 | −4.456 | 4.481 | 1.00 | 22.17 | H | C |
| ATOM | 1257 | CB | PRO | H | 167 | −3.849 | −4.342 | 3.619 | 1.00 | 20.44 | H | C |
| ATOM | 1258 | CG | PRO | H | 167 | −2.826 | −4.983 | 4.424 | 1.00 | 18.54 | H | C |
| ATOM | 1259 | C | PRO | H | 167 | −6.339 | −4.688 | 3.589 | 1.00 | 24.86 | H | C |
| ATOM | 1260 | O | PRO | H | 167 | −6.543 | −5.781 | 3.067 | 1.00 | 22.78 | H | O |
| ATOM | 1261 | N | ALA | H | 168 | −7.149 | −3.661 | 3.402 | 1.00 | 20.24 | H | N |
| ATOM | 1262 | CA | ALA | H | 168 | −8.348 | −3.805 | 2.591 | 1.00 | 19.92 | H | C |
| ATOM | 1263 | CB | ALA | H | 168 | −9.252 | −2.591 | 2.785 | 1.00 | 14.77 | H | C |
| ATOM | 1264 | C | ALA | H | 168 | −8.011 | −3.946 | 1.126 | 1.00 | 22.59 | H | C |
| ATOM | 1265 | O | ALA | H | 168 | −6.950 | −3.514 | 0.688 | 1.00 | 33.99 | H | O |
| ATOM | 1266 | N | VAL | H | 169 | −8.916 | −4.551 | 0.367 | 1.00 | 22.04 | H | N |
| ATOM | 1267 | CA | VAL | H | 169 | −8.724 | −4.680 | −1.064 | 1.00 | 24.94 | H | C |
| ATOM | 1268 | CB | VAL | H | 169 | −8.500 | −6.149 | −1.497 | 1.00 | 30.52 | H | C |
| ATOM | 1269 | CG1 | VAL | H | 169 | −7.255 | −6.731 | −0.792 | 1.00 | 22.01 | H | C |
| ATOM | 1270 | CG2 | VAL | H | 169 | −9.749 | −6.987 | −1.223 | 1.00 | 32.63 | H | C |
| ATOM | 1271 | C | VAL | H | 169 | −9.932 | −4.097 | −1.793 | 1.00 | 25.83 | H | C |
| ATOM | 1272 | O | VAL | H | 169 | −11.053 | −4.154 | −1.298 | 1.00 | 29.00 | H | O |
| ATOM | 1273 | N | LEU | H | 170 | −9.681 | −3.475 | −2.939 | 1.00 | 28.46 | H | N |
| ATOM | 1274 | CA | LEU | H | 170 | −10.735 | −2.881 | −3.756 | 1.00 | 27.34 | H | C |
| ATOM | 1275 | CB | LEU | H | 170 | −10.182 | −1.664 | −4.507 | 1.00 | 22.62 | H | C |
| ATOM | 1276 | CG | LEU | H | 170 | −11.061 | −0.836 | −5.464 | 1.00 | 26.17 | H | C |
| ATOM | 1277 | CD1 | LEU | H | 170 | −12.318 | −0.307 | −4.752 | 1.00 | 12.70 | H | C |
| ATOM | 1278 | CD2 | LEU | H | 170 | −10.229 | 0.347 | −6.027 | 1.00 | 17.11 | H | C |
| ATOM | 1279 | C | LEU | H | 170 | −11.260 | −3.919 | −4.755 | 1.00 | 29.67 | H | C |
| ATOM | 1280 | O | LEU | H | 170 | −10.529 | −4.335 | −5.650 | 1.00 | 30.11 | H | O |
| ATOM | 1281 | N | GLN | H | 171 | −12.503 | −4.366 | −4.564 | 1.00 | 24.66 | H | N |
| ATOM | 1282 | CA | GLN | H | 171 | −13.134 | −5.344 | −5.456 | 1.00 | 29.71 | H | C |
| ATOM | 1283 | CB | GLN | H | 171 | −14.329 | −6.020 | −4.757 | 1.00 | 33.06 | H | C |
| ATOM | 1284 | CG | GLN | H | 171 | −13.953 | −7.072 | −3.725 | 1.00 | 30.84 | H | C |
| ATOM | 1285 | CD | GLN | H | 171 | −14.913 | −7.116 | −2.552 | 1.00 | 33.63 | H | C |
| ATOM | 1286 | OE1 | GLN | H | 171 | −15.183 | −8.180 | −1.987 | 1.00 | 33.72 | H | O |
| ATOM | 1287 | NE2 | GLN | H | 171 | −15.419 | −5.957 | −2.169 | 1.00 | 28.71 | H | N |
| ATOM | 1288 | C | GLN | H | 171 | −13.616 | −4.649 | −6.719 | 1.00 | 29.84 | H | C |
| ATOM | 1289 | O | GLN | H | 171 | −13.799 | −3.425 | −6.722 | 1.00 | 31.80 | H | O |
| ATOM | 1290 | N | SER | H | 172 | −13.863 | −5.418 | −7.781 | 1.00 | 30.57 | H | N |
| ATOM | 1291 | CA | SER | H | 172 | −14.335 | −4.815 | −9.041 | 1.00 | 34.74 | H | C |
| ATOM | 1292 | CB | SER | H | 172 | −14.368 | −5.838 | −10.186 | 1.00 | 31.75 | H | C |
| ATOM | 1293 | OG | SER | H | 172 | −15.169 | −6.965 | −9.873 | 1.00 | 40.37 | H | O |
| ATOM | 1294 | C | SER | H | 172 | −15.686 | −4.086 | −8.921 | 1.00 | 32.56 | H | C |
| ATOM | 1295 | O | SER | H | 172 | −16.098 | −3.381 | −9.840 | 1.00 | 35.53 | H | O |
| ATOM | 1296 | N | SER | H | 173 | −16.336 | −4.213 | −7.764 | 1.00 | 33.29 | H | N |
| ATOM | 1297 | CA | SER | H | 173 | −17.627 | −3.566 | −7.501 | 1.00 | 28.86 | H | C |
| ATOM | 1298 | CB | SER | H | 173 | −18.400 | −4.338 | −6.434 | 1.00 | 24.16 | H | C |
| ATOM | 1299 | OG | SER | H | 173 | −17.759 | −4.230 | −5.172 | 1.00 | 28.86 | H | O |
| ATOM | 1300 | C | SER | H | 173 | −17.456 | −2.136 | −6.998 | 1.00 | 28.91 | H | C |
| ATOM | 1301 | O | SER | H | 173 | −18.449 | −1.413 | −6.820 | 1.00 | 28.04 | H | O |
| ATOM | 1302 | N | GLY | H | 174 | −16.205 | −1.766 | −6.716 | 1.00 | 25.81 | H | N |
| ATOM | 1303 | CA | GLY | H | 174 | −15.889 | −0.445 | −6.196 | 1.00 | 21.79 | H | C |
| ATOM | 1304 | C | GLY | H | 174 | −16.016 | −0.368 | −4.674 | 1.00 | 22.59 | H | C |
| ATOM | 1305 | O | GLY | H | 174 | −15.853 | 0.697 | −4.080 | 1.00 | 23.92 | H | O |
| ATOM | 1306 | N | LEU | H | 175 | −16.296 | −1.504 | −4.044 | 1.00 | 15.70 | H | N |
| ATOM | 1307 | CA | LEU | H | 175 | −16.463 | −1.571 | −2.601 | 1.00 | 21.17 | H | C |
| ATOM | 1308 | CB | LEU | H | 175 | −17.768 | −2.303 | −2.259 | 1.00 | 21.02 | H | C |
| ATOM | 1309 | CG | LEU | H | 175 | −19.055 | −1.805 | −2.950 | 1.00 | 28.34 | H | C |
| ATOM | 1310 | CD1 | LEU | H | 175 | −20.238 | −2.748 | −2.656 | 1.00 | 18.97 | H | C |
| ATOM | 1311 | CD2 | LEU | H | 175 | −19.369 | −0.368 | −2.511 | 1.00 | 22.39 | H | C |
| ATOM | 1312 | C | LEU | H | 175 | −15.276 | −2.297 | −1.979 | 1.00 | 25.03 | H | C |
| ATOM | 1313 | O | LEU | H | 175 | −14.587 | −3.073 | −2.647 | 1.00 | 22.41 | H | O |
| ATOM | 1314 | N | TYR | H | 176 | −15.025 | −2.042 | −0.701 | 1.00 | 24.98 | H | N |
| ATOM | 1315 | CA | TYR | H | 176 | −13.904 | −2.683 | −0.029 | 1.00 | 27.12 | H | C |
| ATOM | 1316 | CB | TYR | H | 176 | −13.137 | −1.688 | 0.827 | 1.00 | 21.90 | H | C |
| ATOM | 1317 | CG | TYR | H | 176 | −12.426 | −0.637 | 0.034 | 1.00 | 22.62 | H | C |
| ATOM | 1318 | CD1 | TYR | H | 176 | −13.076 | 0.535 | −0.323 | 1.00 | 19.07 | H | C |
| ATOM | 1319 | CE1 | TYR | H | 176 | −12.415 | 1.552 | −0.973 | 1.00 | 22.30 | H | C |
| ATOM | 1320 | CD2 | TYR | H | 176 | −11.077 | −0.774 | −0.296 | 1.00 | 25.83 | H | C |
| ATOM | 1321 | CE2 | TYR | H | 176 | −10.392 | 0.249 | −0.957 | 1.00 | 24.62 | H | C |
| ATOM | 1322 | CZ | TYR | H | 176 | −11.075 | 1.413 | −1.286 | 1.00 | 25.87 | H | C |
| ATOM | 1323 | OH | TYR | H | 176 | −10.430 | 2.466 | −1.893 | 1.00 | 31.73 | H | O |
| ATOM | 1324 | C | TYR | H | 176 | −14.268 | −3.899 | 0.799 | 1.00 | 27.29 | H | C |
| ATOM | 1325 | O | TYR | H | 176 | −15.388 | −4.034 | 1.285 | 1.00 | 28.61 | H | O |
| ATOM | 1326 | N | SER | H | 177 | −13.281 | −4.762 | 0.990 | 1.00 | 28.85 | H | N |
| ATOM | 1327 | CA | SER | H | 177 | −13.465 | −5.979 | 1.746 | 1.00 | 29.35 | H | C |
| ATOM | 1328 | CB | SER | H | 177 | −13.713 | −7.136 | 0.797 | 1.00 | 32.91 | H | C |
| ATOM | 1329 | OG | SER | H | 177 | −14.456 | −8.147 | 1.430 | 1.00 | 45.72 | H | O |
| ATOM | 1330 | C | SER | H | 177 | −12.192 | −6.192 | 2.538 | 1.00 | 30.06 | H | C |
| ATOM | 1331 | O | SER | H | 177 | −11.099 | −5.865 | 2.084 | 1.00 | 30.74 | H | O |
| ATOM | 1332 | N | LEU | H | 178 | −12.336 | −6.778 | 3.714 | 1.00 | 34.36 | H | N |
| ATOM | 1333 | CA | LEU | H | 178 | −11.208 | −6.979 | 4.604 | 1.00 | 32.28 | H | C |
| ATOM | 1334 | CB | LEU | H | 178 | −10.952 | −5.646 | 5.315 | 1.00 | 31.46 | H | C |
| ATOM | 1335 | CG | LEU | H | 178 | −10.278 | −5.451 | 6.669 | 1.00 | 28.66 | H | C |
| ATOM | 1336 | CD1 | LEU | H | 178 | −9.917 | −3.987 | 6.795 | 1.00 | 24.09 | H | C |
| ATOM | 1337 | CD2 | LEU | H | 178 | −11.179 | −5.872 | 7.797 | 1.00 | 26.76 | H | C |
| ATOM | 1338 | C | LEU | H | 178 | −11.498 | −8.080 | 5.613 | 1.00 | 36.17 | H | C |
| ATOM | 1339 | O | LEU | H | 178 | −12.654 | −8.336 | 5.952 | 1.00 | 40.19 | H | O |
| ATOM | 1340 | N | SER | H | 179 | −10.440 | −8.698 | 6.122 | 1.00 | 35.36 | H | N |
| ATOM | 1341 | CA | SER | H | 179 | −10.589 | −9.749 | 7.102 | 1.00 | 34.02 | H | C |
| ATOM | 1342 | CB | SER | H | 179 | −10.322 | −11.115 | 6.487 | 1.00 | 34.67 | H | C |
| ATOM | 1343 | OG | SER | H | 179 | −11.393 | −11.500 | 5.644 | 1.00 | 47.45 | H | O |
| ATOM | 1344 | C | SER | H | 179 | −9.670 | −9.560 | 8.280 | 1.00 | 33.84 | H | C |
| ATOM | 1345 | O | SER | H | 179 | −8.540 | −9.107 | 8.136 | 1.00 | 31.68 | H | O |
| ATOM | 1346 | N | SER | H | 180 | −10.186 | −9.901 | 9.454 | 1.00 | 31.92 | H | N |
| ATOM | 1347 | CA | SER | H | 180 | −9.429 | −9.834 | 10.686 | 1.00 | 28.11 | H | C |
| ATOM | 1348 | CB | SER | H | 180 | −10.031 | −8.833 | 11.653 | 1.00 | 28.24 | H | C |
| ATOM | 1349 | OG | SER | H | 180 | −9.249 | −8.812 | 12.826 | 1.00 | 24.47 | H | O |
| ATOM | 1350 | C | SER | H | 180 | −9.499 | −11.213 | 11.305 | 1.00 | 28.64 | H | C |
| ATOM | 1351 | O | SER | H | 180 | −10.582 | −11.741 | 11.528 | 1.00 | 28.87 | H | O |
| ATOM | 1352 | N | VAL | H | 181 | −8.348 | −11.812 | 11.557 | 1.00 | 24.67 | H | N |
| ATOM | 1353 | CA | VAL | H | 181 | −8.335 | −13.128 | 12.145 | 1.00 | 23.23 | H | C |
| ATOM | 1354 | CB | VAL | H | 181 | −7.710 | −14.167 | 11.181 | 1.00 | 25.51 | H | C |
| ATOM | 1355 | CG1 | VAL | H | 181 | −8.360 | −14.053 | 9.800 | 1.00 | 24.54 | H | C |
| ATOM | 1356 | CG2 | VAL | H | 181 | −6.203 | −13.972 | 11.068 | 1.00 | 19.02 | H | C |
| ATOM | 1357 | C | VAL | H | 181 | −7.547 | −13.097 | 13.439 | 1.00 | 30.75 | H | C |
| ATOM | 1358 | O | VAL | H | 181 | −6.849 | −12.116 | 13.738 | 1.00 | 32.48 | H | O |
| ATOM | 1359 | N | VAL | H | 182 | −7.699 | −14.157 | 14.227 | 1.00 | 31.77 | H | N |
| ATOM | 1360 | CA | VAL | H | 182 | −6.958 | −14.297 | 15.473 | 1.00 | 29.90 | H | C |
| ATOM | 1361 | CB | VAL | H | 182 | −7.630 | −13.532 | 16.660 | 1.00 | 34.23 | H | C |
| ATOM | 1362 | CG1 | VAL | H | 182 | −8.962 | −14.172 | 17.040 | 1.00 | 29.16 | H | C |
| ATOM | 1363 | CG2 | VAL | H | 182 | −6.672 | −13.459 | 17.853 | 1.00 | 26.52 | H | C |
| ATOM | 1364 | C | VAL | H | 182 | −6.768 | −15.785 | 15.775 | 1.00 | 30.91 | H | C |
| ATOM | 1365 | O | VAL | H | 182 | −7.604 | −16.614 | 15.392 | 1.00 | 29.82 | H | O |
| ATOM | 1366 | N | THR | H | 183 | −5.583 | −16.135 | 16.270 | 1.00 | 26.10 | H | N |
| ATOM | 1367 | CA | THR | H | 183 | −5.301 | −17.523 | 16.622 | 1.00 | 31.12 | H | C |
| ATOM | 1368 | CB | THR | H | 183 | −3.901 | −18.036 | 16.132 | 1.00 | 33.02 | H | C |
| ATOM | 1369 | OG1 | THR | H | 183 | −2.884 | −17.074 | 16.433 | 1.00 | 37.57 | H | O |
| ATOM | 1370 | CG2 | THR | H | 183 | −3.901 | −18.326 | 14.634 | 1.00 | 32.38 | H | C |
| ATOM | 1371 | C | THR | H | 183 | −5.395 | −17.611 | 18.137 | 1.00 | 32.60 | H | C |
| ATOM | 1372 | O | THR | H | 183 | −4.870 | −16.756 | 18.865 | 1.00 | 35.42 | H | O |
| ATOM | 1373 | N | VAL | H | 184 | −6.139 | −18.604 | 18.601 | 1.00 | 35.35 | H | N |
| ATOM | 1374 | CA | VAL | H | 184 | −6.352 | −18.824 | 20.024 | 1.00 | 38.14 | H | C |
| ATOM | 1375 | CB | VAL | H | 184 | −7.781 | −18.380 | 29.433 | 1.00 | 40.53 | H | C |
| ATOM | 1376 | CG1 | VAL | H | 184 | −7.888 | −16.857 | 20.445 | 1.00 | 33.86 | H | C |
| ATOM | 1377 | CG2 | VAL | H | 184 | −8.822 | −18.993 | 19.477 | 1.00 | 37.66 | H | C |
| ATOM | 1378 | C | VAL | H | 184 | −6.188 | −20.306 | 20.349 | 1.00 | 40.83 | H | C |
| ATOM | 1379 | O | VAL | H | 184 | −6.098 | −21.142 | 19.444 | 1.00 | 37.25 | H | O |
| ATOM | 1380 | N | PRO | H | 185 | −6.050 | −20.644 | 21.641 | 1.00 | 44.07 | H | N |
| ATOM | 1381 | CD | PRO | H | 185 | −5.732 | −19.788 | 22.798 | 1.00 | 45.51 | H | C |
| ATOM | 1382 | CA | PRO | H | 185 | −5.904 | −22.069 | 21.968 | 1.00 | 44.93 | H | C |
| ATOM | 1383 | CB | PRO | H | 185 | −5.517 | −22.051 | 23.449 | 1.00 | 44.45 | H | C |
| ATOM | 1384 | CG | PRO | H | 185 | −6.003 | −20.701 | 23.943 | 1.00 | 46.43 | H | C |
| ATOM | 1385 | C | PRO | H | 185 | −7.219 | −22.816 | 21.697 | 1.00 | 46.95 | H | C |
| ATOM | 1386 | O | PRO | H | 185 | −8.305 | −22.256 | 21.883 | 1.00 | 48.84 | H | O |
| ATOM | 1387 | N | SER | H | 186 | −7.112 | −24.043 | 21.186 | 1.00 | 46.49 | H | N |
| ATOM | 1388 | CA | SER | H | 186 | −8.278 | −24.873 | 20.862 | 1.00 | 50.16 | H | C |
| ATOM | 1389 | CB | SER | H | 186 | −7.828 | −26.200 | 20.267 | 1.00 | 49.36 | H | C |
| ATOM | 1390 | OG | SER | H | 186 | −6.891 | −26.008 | 19.230 | 1.00 | 57.99 | H | O |
| ATOM | 1391 | C | SER | H | 186 | −9.184 | −25.182 | 22.056 | 1.00 | 54.57 | H | C |
| ATOM | 1392 | O | SER | H | 186 | −10.389 | −25.372 | 21.901 | 1.00 | 56.74 | H | O |
| ATOM | 1393 | N | SER | H | 187 | −8.591 | −25.256 | 23.243 | 1.00 | 57.90 | H | N |
| ATOM | 1394 | CA | SER | H | 187 | −9.329 | −25.577 | 24.460 | 1.00 | 58.49 | H | C |
| ATOM | 1395 | CB | SER | H | 187 | −8.356 | −25.843 | 25.614 | 1.00 | 56.26 | H | C |
| ATOM | 1396 | OG | SER | H | 187 | −7.553 | −24.709 | 25.881 | 1.00 | 50.01 | H | O |
| ATOM | 1397 | C | SER | H | 187 | −10.350 | −24.525 | 24.877 | 1.00 | 60.02 | H | C |
| ATOM | 1398 | O | SER | H | 187 | −11.410 | −24.857 | 25.406 | 1.00 | 65.53 | H | O |
| ATOM | 1399 | N | SER | H | 188 | −10.033 | −23.264 | 24.621 | 1.00 | 60.83 | H | N |
| ATOM | 1400 | CA | SER | H | 188 | −10.902 | −22.154 | 24.979 | 1.00 | 57.52 | H | C |
| ATOM | 1401 | CB | SER | H | 188 | −10.112 | −20.844 | 24.908 | 1.00 | 60.92 | H | C |
| ATOM | 1402 | OG | SER | H | 188 | −9.646 | −20.595 | 23.588 | 1.00 | 61.04 | H | O |
| ATOM | 1403 | C | SER | H | 188 | −12.182 | −22.029 | 24.146 | 1.00 | 59.63 | H | C |
| ATOM | 1404 | O | SER | H | 188 | −13.043 | −21.202 | 24.464 | 1.00 | 64.60 | H | O |
| ATOM | 1405 | N | LEU | H | 189 | −12.306 | −22.823 | 23.081 | 1.00 | 56.50 | H | N |
| ATOM | 1406 | CA | LEU | H | 189 | −13.493 | −22.768 | 22.218 | 1.00 | 55.91 | H | C |
| ATOM | 1407 | CB | LEU | H | 189 | −13.286 | −23.598 | 20.942 | 1.00 | 51.20 | H | C |
| ATOM | 1408 | CG | LEU | H | 189 | −12.145 | −23.181 | 19.999 | 1.00 | 51.05 | H | C |
| ATOM | 1409 | CD1 | LEU | H | 189 | −12.159 | −24.082 | 18.765 | 1.00 | 43.55 | H | C |
| ATOM | 1410 | CD2 | LEU | H | 189 | −12.261 | −21.702 | 19.605 | 1.00 | 39.59 | H | C |
| ATOM | 1411 | C | LEU | H | 189 | −14.744 | −23.231 | 22.970 | 1.00 | 59.71 | H | C |
| ATOM | 1412 | O | LEU | H | 189 | −15.052 | −24.427 | 23.045 | 1.00 | 62.48 | H | O |
| ATOM | 1413 | N | GLY | H | 190 | −15.465 | −22.257 | 23.510 | 1.00 | 60.62 | H | N |
| ATOM | 1414 | CA | GLY | H | 190 | −16.655 | −22.533 | 24.292 | 1.00 | 60.44 | H | C |
| ATOM | 1415 | C | GLY | H | 190 | −16.512 | −21.734 | 25.578 | 1.00 | 59.68 | H | C |
| ATOM | 1416 | O | GLY | H | 190 | −17.330 | −20.856 | 25.875 | 1.00 | 58.15 | H | O |
| ATOM | 1417 | N | THR | H | 191 | −15.429 | −21.999 | 26.306 | 1.00 | 57.98 | H | N |
| ATOM | 1418 | CA | THR | H | 191 | −15.132 | −21.304 | 27.562 | 1.00 | 57.45 | H | C |
| ATOM | 1419 | CB | THR | H | 191 | −13.802 | −21.808 | 28.176 | 1.00 | 56.58 | H | C |
| ATOM | 1420 | OG1 | THR | H | 191 | −13.864 | −23.228 | 28.355 | 1.00 | 57.35 | H | O |
| ATOM | 1421 | CG2 | THR | H | 191 | −13.533 | −21.134 | 29.526 | 1.00 | 54.93 | H | C |
| ATOM | 1422 | C | THR | H | 191 | −15.022 | −19.787 | 27.359 | 1.00 | 55.61 | H | C |
| ATOM | 1423 | O | THR | H | 191 | −15.657 | −19.014 | 28.076 | 1.00 | 60.05 | H | O |
| ATOM | 1424 | N | GLN | H | 192 | −14.206 | −19.371 | 26.391 | 1.00 | 54.70 | H | N |
| ATOM | 1425 | CA | GLN | H | 192 | −14.002 | −17.948 | 26.095 | 1.00 | 49.39 | H | C |
| ATOM | 1426 | CB | GLN | H | 192 | −12.517 | −17.665 | 25.875 | 1.00 | 47.99 | H | C |
| ATOM | 1427 | CG | GLN | H | 192 | −12.216 | −16.295 | 25.322 | 1.00 | 48.63 | H | C |
| ATOM | 1428 | CD | GLN | H | 192 | −12.247 | −15.207 | 26.364 | 1.00 | 53.56 | H | C |
| ATOM | 1429 | OE1 | GLN | H | 192 | −11.284 | −15.027 | 27.115 | 1.00 | 57.51 | H | O |
| ATOM | 1430 | NE2 | GLN | H | 192 | −13.336 | −14.447 | 26.398 | 1.00 | 50.56 | H | N |
| ATOM | 1431 | C | GLN | H | 192 | −14.831 | −17.431 | 24.907 | 1.00 | 47.58 | H | C |
| ATOM | 1432 | O | GLN | H | 192 | −14.968 | −18.093 | 23.878 | 1.00 | 49.86 | H | O |
| ATOM | 1433 | N | THR | H | 193 | −15.393 | −16.243 | 25.085 | 1.00 | 44.84 | H | N |
| ATOM | 1434 | CA | THR | H | 193 | −16.216 | −15.587 | 24.082 | 1.00 | 46.87 | H | C |
| ATOM | 1435 | CB | THR | H | 193 | −17.414 | −14.848 | 24.779 | 1.00 | 47.38 | H | C |
| ATOM | 1436 | OG1 | THR | H | 193 | −18.517 | −15.753 | 24.916 | 1.00 | 49.87 | H | O |
| ATOM | 1437 | CG2 | THR | H | 193 | −17.876 | −13.622 | 23.999 | 1.00 | 46.62 | H | C |
| ATOM | 1438 | C | THR | H | 193 | −15.403 | −14.610 | 23.216 | 1.00 | 46.48 | H | C |
| ATOM | 1439 | O | THR | H | 193 | −14.786 | −13.673 | 23.734 | 1.00 | 47.43 | H | O |
| ATOM | 1440 | N | TYR | H | 194 | −15.413 | −14.835 | 21.900 | 1.00 | 45.24 | H | N |
| ATOM | 1441 | CA | TYR | H | 194 | −14.696 | −13.972 | 20.952 | 1.00 | 40.94 | H | C |
| ATOM | 1442 | CB | TYR | H | 194 | −13.786 | −14.807 | 20.049 | 1.00 | 33.22 | H | C |
| ATOM | 1443 | CG | TYR | H | 194 | −12.750 | −15.587 | 20.822 | 1.00 | 34.45 | H | C |
| ATOM | 1444 | CD1 | TYR | H | 194 | −11.722 | −14.933 | 21.505 | 1.00 | 33.10 | H | C |
| ATOM | 1445 | CE1 | TYR | H | 194 | −10.772 | −15.647 | 22.223 | 1.00 | 33.39 | H | C |
| ATOM | 1446 | CD2 | TYR | H | 194 | −12.803 | −16.977 | 20.878 | 1.00 | 31.44 | H | C |
| ATOM | 1447 | CE2 | TYR | H | 194 | −11.858 | −17.704 | 21.587 | 1.00 | 36.06 | H | C |
| ATOM | 1448 | CZ | TYR | H | 194 | −10.843 | −17.037 | 22.259 | 1.00 | 37.15 | H | C |
| ATOM | 1449 | OH | TYR | H | 194 | −9.903 | −17.765 | 22.963 | 1.00 | 32.86 | H | O |
| ATOM | 1450 | C | TYR | H | 194 | −15.668 | −13.149 | 20.111 | 1.00 | 39.18 | H | C |
| ATOM | 1451 | O | TYR | H | 194 | −16.516 | −13.700 | 19.402 | 1.00 | 33.12 | H | O |
| ATOM | 1452 | N | ILE | H | 195 | −15.539 | −11.830 | 20.214 | 1.00 | 36.80 | H | N |
| ATOM | 1453 | CA | ILE | H | 195 | −16.396 | −10.896 | 19.494 | 1.00 | 37.16 | H | C |
| ATOM | 1454 | CB | ILE | H | 195 | −17.237 | −10.033 | 20.474 | 1.00 | 37.64 | H | C |
| ATOM | 1455 | CG2 | ILE | H | 195 | −18.016 | −8.963 | 19.718 | 1.00 | 36.16 | H | C |
| ATOM | 1456 | CG1 | ILE | H | 195 | −18.172 | −10.884 | 21.326 | 1.00 | 35.68 | H | C |
| ATOM | 1457 | CD1 | ILE | H | 195 | −18.868 | −10.062 | 22.427 | 1.00 | 34.92 | H | C |
| ATOM | 1458 | C | ILE | H | 195 | −15.510 | −9.907 | 18.748 | 1.00 | 41.74 | H | C |
| ATOM | 1459 | O | ILE | H | 195 | −14.599 | −9.328 | 19.344 | 1.00 | 41.06 | H | O |
| ATOM | 1460 | N | CYS | H | 196 | −15.776 | −9.698 | 17.459 | 1.00 | 44.09 | H | N |
| ATOM | 1461 | CA | CYS | H | 196 | −15.001 | −8.719 | 16.706 | 1.00 | 44.51 | H | C |
| ATOM | 1462 | C | CYS | H | 196 | −15.841 | −7.443 | 16.623 | 1.00 | 42.10 | H | C |
| ATOM | 1463 | O | CYS | H | 196 | −17.033 | −7.480 | 16.296 | 1.00 | 38.46 | H | O |
| ATOM | 1464 | CB | CYS | H | 196 | −14.601 | −9.230 | 15.309 | 1.00 | 46.76 | H | C |
| ATOM | 1465 | SG | CYS | H | 196 | −15.895 | −9.192 | 14.034 | 1.00 | 52.49 | H | S |
| ATOM | 1466 | N | ASN | H | 197 | −15.220 | −6.326 | 16.976 | 1.00 | 36.20 | H | N |
| ATOM | 1467 | CA | ASN | H | 197 | −15.888 | −5.036 | 16.971 | 1.00 | 39.39 | H | C |
| ATOM | 1468 | CB | ASN | H | 197 | −15.491 | −4.237 | 18.216 | 1.00 | 41.52 | H | C |
| ATOM | 1469 | CG | ASN | H | 197 | −15.398 | 5.103 | 19.448 | 1.00 | 41.07 | H | C |
| ATOM | 1470 | OD1 | ASN | H | 197 | −14.322 | −5.265 | 20.022 | 1.00 | 46.69 | H | O |
| ATOM | 1471 | ND2 | ASN | H | 197 | −16.517 | −5.700 | 19.839 | 1.00 | 43.94 | H | N |
| ATOM | 1472 | C | ASN | H | 197 | −15.534 | −4.251 | 15.722 | 1.00 | 34.09 | H | C |
| ATOM | 1473 | O | ASN | H | 197 | −14.442 | −3.701 | 15.612 | 1.00 | 42.47 | H | O |
| ATOM | 1474 | N | VAL | H | 198 | −16.494 | −4.157 | 14.811 | 1.00 | 30.96 | H | N |
| ATOM | 1475 | CA | VAL | H | 198 | −16.298 | −3.452 | 13.561 | 1.00 | 21.85 | H | C |
| ATOM | 1476 | CB | VAL | H | 198 | −16.873 | −4.264 | 12.410 | 1.00 | 20.68 | H | C |
| ATOM | 1477 | CG1 | VAL | H | 198 | −16.585 | −3.575 | 11.096 | 1.00 | 24.17 | H | C |
| ATOM | 1478 | CG2 | VAL | H | 198 | −16.310 | −5.696 | 12.440 | 1.00 | 16.33 | H | C |
| ATOM | 1479 | C | VAL | H | 198 | −16.956 | −2.085 | 13.585 | 1.00 | 27.39 | H | C |
| ATOM | 1480 | O | VAL | H | 198 | −18.107 | −1.943 | 14.002 | 1.00 | 41.75 | H | O |
| ATOM | 1481 | N | ASN | H | 199 | −16.239 | −1.077 | 13.112 | 1.00 | 24.81 | H | N |
| ATOM | 1482 | CA | ASN | H | 199 | −16.761 | 0.272 | 13.089 | 1.00 | 25.01 | H | C |
| ATOM | 1483 | CB | ASN | H | 199 | −16.187 | 1.079 | 14.263 | 1.00 | 36.72 | H | C |
| ATOM | 1484 | CG | ASN | H | 199 | −16.810 | 2.467 | 14.388 | 1.00 | 43.98 | H | C |
| ATOM | 1485 | OD1 | ASN | H | 199 | −16.164 | 3.408 | 14.851 | 1.00 | 50.61 | H | O |
| ATOM | 1486 | ND2 | ASN | H | 199 | −18.071 | 2.598 | 13.975 | 1.00 | 51.98 | H | N |
| ATOM | 1487 | C | ASN | H | 199 | −16.421 | 0.941 | 11.765 | 1.00 | 29.80 | H | C |
| ATOM | 1488 | O | ASN | H | 199 | −15.274 | 0.915 | 11.314 | 1.00 | 34.43 | H | O |
| ATOM | 1489 | N | HIS | H | 200 | −17.432 | 1.539 | 11.147 | 1.00 | 26.41 | H | N |
| ATOM | 1490 | CA | HIS | H | 200 | −17.280 | 2.224 | 9.873 | 1.00 | 29.01 | H | C |
| ATOM | 1491 | CB | HIS | H | 200 | −17.927 | 1.403 | 8.748 | 1.00 | 24.43 | H | C |
| ATOM | 1492 | CG | HIS | H | 200 | −17.777 | 2.010 | 7.387 | 1.00 | 27.74 | H | C |
| ATOM | 1493 | CD2 | HIS | H | 200 | −16.683 | 2.220 | 6.619 | 1.00 | 26.67 | H | C |
| ATOM | 1494 | ND1 | HIS | H | 200 | −18.854 | 2.472 | 6.656 | 1.00 | 26.82 | H | N |
| ATOM | 1495 | CE1 | HIS | H | 200 | −18.428 | 2.940 | 5.496 | 1.00 | 25.21 | H | C |
| ATOM | 1496 | NE2 | HIS | H | 200 | −17.113 | 2.798 | 5.447 | 1.00 | 29.16 | H | N |
| ATOM | 1497 | C | HIS | H | 200 | −17.980 | 3.555 | 10.060 | 1.00 | 28.37 | H | C |
| ATOM | 1498 | O | HIS | H | 200 | −19.186 | 3.674 | 9.871 | 1.00 | 30.09 | H | O |
| ATOM | 1499 | N | LYS | H | 201 | −17.203 | 4.565 | 10.425 | 1.00 | 32.19 | H | N |
| ATOM | 1500 | CA | LYS | H | 201 | −17.757 | 5.881 | 10.694 | 1.00 | 33.75 | H | C |
| ATOM | 1501 | CB | LYS | H | 201 | −16.675 | 6.834 | 11.237 | 1.00 | 36.86 | H | C |
| ATOM | 1502 | CG | LYS | H | 201 | −16.000 | 6.396 | 12.544 | 1.00 | 42.02 | H | C |
| ATOM | 1503 | CD | LYS | H | 201 | −17.018 | 6.064 | 13.655 | 1.00 | 55.03 | H | C |
| ATOM | 1504 | CE | LYS | H | 201 | −16.620 | 6.655 | 15.030 | 1.00 | 59.17 | H | C |
| ATOM | 1505 | NZ | LYS | H | 201 | −15.224 | 6.298 | 15.455 | 1.00 | 60.26 | H | N |
| ATOM | 1506 | C | LYS | H | 201 | −18.600 | 6.587 | 9.624 | 1.00 | 29.63 | H | C |
| ATOM | 1507 | O | LYS | H | 201 | −19.656 | 7.118 | 9.945 | 1.00 | 36.80 | H | O |
| ATOM | 1508 | N | PRO | H | 202 | −18.203 | 6.537 | 8.336 | 1.00 | 28.91 | H | N |
| ATOM | 1509 | CD | PRO | H | 202 | −17.090 | 5.813 | 7.700 | 1.00 | 25.36 | H | C |
| ATOM | 1510 | CA | PRO | H | 202 | −19.005 | 7.232 | 7.316 | 1.00 | 25.16 | H | C |
| ATOM | 1511 | CB | PRO | H | 202 | −18.216 | 6.992 | 6.035 | 1.00 | 24.87 | H | C |
| ATOM | 1512 | CG | PRO | H | 202 | −16.831 | 6.671 | 6.510 | 1.00 | 17.52 | H | C |
| ATOM | 1513 | C | PRO | H | 202 | −20.465 | 6.797 | 7.165 | 1.00 | 27.51 | H | C |
| ATOM | 1514 | O | PRO | H | 202 | −21.293 | 7.566 | 6.687 | 1.00 | 29.47 | H | O |
| ATOM | 1515 | N | SER | H | 203 | −20.775 | 5.562 | 7.547 | 1.00 | 27.88 | H | N |
| ATOM | 1516 | CA | SER | H | 203 | −22.142 | 5.062 | 7.463 | 1.00 | 25.61 | H | C |
| ATOM | 1517 | CB | SER | H | 203 | −22.193 | 3.747 | 6.669 | 1.00 | 21.25 | H | C |
| ATOM | 1518 | OG | SER | H | 203 | −21.661 | 2.660 | 7.430 | 1.00 | 18.60 | H | O |
| ATOM | 1519 | C | SER | H | 203 | −22.740 | 4.837 | 8.865 | 1.00 | 30.06 | H | C |
| ATOM | 1520 | O | SER | H | 203 | −23.846 | 4.298 | 8.985 | 1.00 | 27.56 | H | O |
| ATOM | 1521 | N | ASN | H | 204 | −22.005 | 5.221 | 9.914 | 1.00 | 24.18 | H | N |
| ATOM | 1522 | CA | ASN | H | 204 | −22.472 | 5.055 | 11.286 | 1.00 | 24.25 | H | C |
| ATOM | 1523 | CB | ASN | H | 204 | −23.763 | 5.864 | 11.544 | 1.00 | 23.13 | H | C |
| ATOM | 1524 | CG | ASN | H | 204 | −23.502 | 7.332 | 11.778 | 1.00 | 29.02 | H | C |
| ATOM | 1525 | OD1 | ASN | H | 204 | −23.823 | 8.182 | 10.946 | 1.00 | 27.00 | H | O |
| ATOM | 1526 | ND2 | ASN | H | 204 | −22.925 | 7.643 | 12.929 | 1.00 | 34.05 | H | N |
| ATOM | 1527 | C | ASN | H | 204 | −22.761 | 3.593 | 11.555 | 1.00 | 26.50 | H | C |
| ATOM | 1528 | O | ASN | H | 204 | −23.782 | 3.253 | 12.143 | 1.00 | 35.22 | H | O |
| ATOM | 1529 | N | THR | H | 205 | −21.876 | 2.720 | 11.104 | 1.00 | 33.14 | H | N |
| ATOM | 1530 | CA | THR | H | 205 | −22.070 | 1.293 | 11.306 | 1.00 | 32.68 | H | C |
| ATOM | 1531 | CB | THR | H | 205 | −21.774 | 0.508 | 10.015 | 1.00 | 31.22 | H | C |
| ATOM | 1532 | OG1 | THR | H | 205 | −22.760 | 0.839 | 9.035 | 1.00 | 33.00 | H | O |
| ATOM | 1533 | CG2 | THR | H | 205 | −21.782 | −0.990 | 10.254 | 1.00 | 22.07 | H | C |
| ATOM | 1534 | C | THR | H | 205 | −21.195 | 0.782 | 12.423 | 1.00 | 37.27 | H | C |
| ATOM | 1535 | O | THR | H | 205 | −19.982 | 0.959 | 12.408 | 1.00 | 40.69 | H | O |
| ATOM | 1536 | N | LYS | H | 206 | −21.834 | 0.176 | 13.414 | 1.00 | 45.13 | H | N |
| ATOM | 1537 | CA | LYS | H | 206 | −21.148 | −0.403 | 14.561 | 1.00 | 49.75 | H | C |
| ATOM | 1538 | CB | LYS | H | 206 | −21.396 | 0.448 | 15.812 | 1.00 | 51.20 | H | C |
| ATOM | 1539 | CG | LYS | H | 206 | −20.988 | −0.223 | 17.133 | 1.00 | 65.44 | H | C |
| ATOM | 1540 | CD | LYS | H | 206 | −21.462 | 0.585 | 18.358 | 1.00 | 69.28 | H | C |
| ATOM | 1541 | CE | LYS | H | 206 | −21.364 | −0.210 | 19.673 | 1.00 | 73.16 | H | C |
| ATOM | 1542 | NZ | LYS | H | 206 | −22.333 | −1.355 | 19.787 | 1.00 | 71.22 | H | N |
| ATOM | 1543 | C | LYS | H | 206 | −21.735 | −1.808 | 14.713 | 1.00 | 47.21 | H | C |
| ATOM | 1544 | O | LYS | H | 206 | −22.924 | −1.967 | 14.976 | 1.00 | 51.75 | H | O |
| ATOM | 1545 | N | VAL | H | 207 | −20.916 | −2.819 | 14.453 | 1.00 | 46.72 | H | N |
| ATOM | 1546 | CA | VAL | H | 207 | −21.354 | −4.209 | 14.539 | 1.00 | 47.31 | H | C |
| ATOM | 1547 | CB | VAL | H | 207 | −21.331 | −4.906 | 13.158 | 1.00 | 45.09 | H | C |
| ATOM | 1548 | CG1 | VAL | H | 207 | −21.853 | −6.328 | 13.281 | 1.00 | 51.22 | H | C |
| ATOM | 1549 | CG2 | VAL | H | 207 | −22.166 | −4.132 | 12.152 | 1.00 | 46.62 | H | C |
| ATOM | 1550 | C | VAL | H | 207 | −20.470 | −5.005 | 15.486 | 1.00 | 45.84 | H | C |
| ATOM | 1551 | O | VAL | H | 207 | −19.252 | −4.803 | 15.547 | 1.00 | 48.53 | H | O |
| ATOM | 1552 | N | ASP | H | 208 | −21.100 | −5.887 | 16.247 | 1.00 | 44.59 | H | N |
| ATOM | 1553 | CA | ASP | H | 208 | −20.391 | −6.730 | 17.195 | 1.00 | 43.49 | H | C |
| ATOM | 1554 | CB | ASP | H | 208 | −20.771 | −6.361 | 18.633 | 1.00 | 43.32 | H | C |
| ATOM | 1555 | CG | ASP | H | 208 | −20.115 | −5.077 | 19.111 | 1.00 | 44.89 | H | C |
| ATOM | 1556 | OD1 | ASP | H | 208 | −19.479 | −4.364 | 18.311 | 1.00 | 50.61 | H | O |
| ATOM | 1557 | OD2 | ASP | H | 208 | −20.226 | −4.779 | 20.313 | 1.00 | 53.19 | H | O |
| ATOM | 1558 | C | ASP | H | 208 | −20.772 | −8.169 | 16.909 | 1.00 | 42.53 | H | C |
| ATOM | 1559 | O | ASP | H | 208 | −21.795 | −8.655 | 17.393 | 1.00 | 44.16 | H | O |
| ATOM | 1560 | N | LYS | H | 209 | −19.962 | −8.849 | 16.109 | 1.00 | 37.35 | H | N |
| ATOM | 1561 | CA | LYS | H | 209 | −20.272 | −10.223 | 15.771 | 1.00 | 40.88 | H | C |
| ATOM | 1562 | CB | LYS | H | 209 | −19.971 | −10.492 | 14.296 | 1.00 | 41.14 | H | C |
| ATOM | 1563 | CG | LYS | H | 209 | −20.264 | −11.928 | 13.816 | 1.00 | 42.22 | H | C |
| ATOM | 1564 | CD | LYS | H | 209 | −21.761 | −12.210 | 13.677 | 1.00 | 42.58 | H | C |
| ATOM | 1565 | CE | LYS | H | 209 | −22.396 | −11.349 | 12.592 | 1.00 | 46.01 | H | C |
| ATOM | 1566 | NZ | LYS | H | 209 | −23.860 | −11.568 | 12.480 | 1.00 | 46.28 | H | N |
| ATOM | 1567 | C | LYS | H | 209 | −19.580 | −11.262 | 16.640 | 1.00 | 45.16 | H | C |
| ATOM | 1568 | O | LYS | H | 209 | −18.353 | −11.260 | 16.773 | 1.00 | 48.72 | H | O |
| ATOM | 1569 | N | LYS | H | 210 | −20.378 | −12.129 | 17.262 | 1.00 | 46.54 | H | N |
| ATOM | 1570 | CA | LYS | H | 210 | −19.830 | −13.209 | 18.078 | 1.00 | 44.21 | H | C |
| ATOM | 1571 | CB | LYS | H | 210 | −20.859 | −13.757 | 19.074 | 1.00 | 47.94 | H | C |
| ATOM | 1572 | CG | LYS | H | 210 | −20.962 | −12.989 | 20.383 | 1.00 | 42.39 | H | C |
| ATOM | 1573 | CD | LYS | H | 210 | −22.030 | −13.583 | 21.299 | 1.00 | 40.95 | H | C |
| ATOM | 1574 | CE | LYS | H | 210 | −21.813 | −15.074 | 21.555 | 1.00 | 39.22 | H | C |
| ATOM | 1575 | NZ | LYS | H | 210 | −20.511 | −15.369 | 22.216 | 1.00 | 37.80 | H | N |
| ATOM | 1576 | C | LYS | H | 210 | −19.452 | −14.288 | 17.080 | 1.00 | 46.06 | H | C |
| ATOM | 1577 | O | LYS | H | 210 | −20.206 | −14.562 | 16.131 | 1.00 | 45.42 | H | O |
| ATOM | 1578 | N | VAL | H | 211 | −18.276 | −14.877 | 17.276 | 1.00 | 37.58 | H | N |
| ATOM | 1579 | CA | VAL | H | 211 | −17.788 | −15.901 | 16.378 | 1.00 | 32.72 | H | C |
| ATOM | 1580 | CB | VAL | H | 211 | −16.431 | −15.473 | 15.798 | 1.00 | 27.40 | H | C |
| ATOM | 1581 | CG1 | VAL | H | 211 | −15.923 | −16.487 | 14.803 | 1.00 | 28.95 | H | C |
| ATOM | 1582 | CG2 | VAL | H | 211 | −16.574 | −14.138 | 15.138 | 1.00 | 29.54 | H | C |
| ATOM | 1583 | C | VAL | H | 211 | −17.669 | −17.210 | 17.128 | 1.00 | 35.29 | H | C |
| ATOM | 1584 | O | VAL | H | 211 | −16.980 | −17.276 | 18.144 | 1.00 | 38.90 | H | O |
| ATOM | 1585 | N | GLU | H | 212 | −18.317 | −18.257 | 16.613 | 1.00 | 41.76 | H | N |
| ATOM | 1586 | CA | GLU | H | 212 | −18.292 | −19.574 | 17.263 | 1.00 | 45.68 | H | C |
| ATOM | 1587 | CB | GLU | H | 212 | −19.421 | −19.659 | 18.305 | 1.00 | 49.63 | H | C |
| ATOM | 1588 | CG | GLU | H | 212 | −20.822 | −19.876 | 17.707 | 1.00 | 54.15 | H | C |
| ATOM | 1589 | CD | GLU | H | 212 | −21.946 | −19.760 | 18.731 | 1.00 | 58.83 | H | C |
| ATOM | 1590 | OE1 | GLU | H | 212 | −22.149 | −20.714 | 19.517 | 1.00 | 60.59 | H | O |
| ATOM | 1591 | OE2 | GLU | H | 212 | −22.634 | −18.713 | 18.740 | 1.00 | 58.02 | H | O |
| ATOM | 1592 | C | GLU | H | 212 | −18.470 | −20.717 | 16.264 | 1.00 | 46.99 | H | C |
| ATOM | 1593 | O | GLU | H | 212 | −18.919 | −20.499 | 15.136 | 1.00 | 50.11 | H | O |
| ATOM | 1594 | N | PRO | H | 213 | −18.106 | −21.954 | 16.666 | 1.00 | 50.55 | H | N |
| ATOM | 1595 | CD | PRO | H | 213 | −17.342 | −22.318 | 17.878 | 1.00 | 50.01 | H | C |
| ATOM | 1596 | CA | PRO | H | 213 | −18.257 | −23.111 | 15.770 | 1.00 | 52.53 | H | C |
| ATOM | 1597 | CB | PRO | H | 213 | −17.457 | −24.215 | 16.486 | 1.00 | 50.02 | H | C |
| ATOM | 1598 | CG | PRO | H | 213 | −17.510 | −23.822 | 17.932 | 1.00 | 48.45 | H | C |
| ATOM | 1599 | C | PRO | H | 213 | −19.732 | −23.492 | 15.582 | 1.00 | 51.58 | H | C |
| ATOM | 1600 | O | PRO | H | 213 | −20.067 | −24.653 | 15.353 | 1.00 | 59.81 | H | O |
| ATOM | 1601 | N | GLU | I | 1 | 7.175 | 24.212 | 16.870 | 1.00 | 42.62 | I | N |
| ATOM | 1602 | CA | GLU | I | 1 | 8.629 | 24.104 | 16.935 | 1.00 | 42.44 | I | C |
| ATOM | 1603 | CB | GLU | I | 1 | 9.264 | 24.705 | 15.685 | 1.00 | 42.56 | I | C |
| ATOM | 1604 | CG | GLU | I | 1 | 10.784 | 24.561 | 15.636 | 1.00 | 50.65 | I | C |
| ATOM | 1605 | CD | GLU | I | 1 | 11.450 | 25.611 | 14.752 | 1.00 | 54.86 | I | C |
| ATOM | 1606 | OE1 | GLU | I | 1 | 10.849 | 26.694 | 14.544 | 1.00 | 55.52 | I | O |
| ATOM | 1607 | OE2 | GLU | I | 1 | 12.580 | 25.360 | 14.280 | 1.00 | 54.51 | I | O |
| ATOM | 1608 | C | GLU | I | 1 | 9.166 | 24.835 | 18.166 | 1.00 | 37.33 | I | C |
| ATOM | 1609 | O | GLU | I | 1 | 9.044 | 26.056 | 18.277 | 1.00 | 31.90 | I | O |
| ATOM | 1610 | N | VAL | I | 2 | 9.756 | 24.077 | 19.084 | 1.00 | 36.15 | I | N |
| ATOM | 1611 | CA | VAL | I | 2 | 10.321 | 24.640 | 20.302 | 1.00 | 31.90 | I | C |
| ATOM | 1612 | CB | VAL | I | 2 | 10.531 | 23.537 | 21.372 | 1.00 | 29.68 | I | C |
| ATOM | 1613 | CG1 | VAL | I | 2 | 11.234 | 24.106 | 22.597 | 1.00 | 22.47 | I | C |
| ATOM | 1614 | CG2 | VAL | I | 2 | 9.180 | 22.937 | 21.771 | 1.00 | 28.75 | I | C |
| ATOM | 1615 | C | VAL | I | 2 | 11.644 | 25.354 | 20.011 | 1.00 | 31.49 | I | C |
| ATOM | 1616 | O | VAL | I | 2 | 12.469 | 24.866 | 19.244 | 1.00 | 34.56 | I | O |
| ATOM | 1617 | N | GLN | I | 3 | 11.796 | 26.545 | 20.577 | 1.00 | 31.80 | I | N |
| ATOM | 1618 | CA | GLN | I | 3 | 13.006 | 27.355 | 20.425 | 1.00 | 36.31 | I | C |
| ATOM | 1619 | CB | GLN | I | 3 | 12.812 | 28.456 | 19.376 | 1.00 | 40.78 | I | C |
| ATOM | 1620 | CG | GLN | I | 3 | 12.740 | 27.992 | 17.949 | 1.00 | 48.48 | I | C |
| ATOM | 1621 | CD | GLN | I | 3 | 12.308 | 29.107 | 17.008 | 1.00 | 54.93 | I | C |
| ATOM | 1622 | OE1 | GLN | I | 3 | 13.148 | 29.778 | 16.405 | 1.00 | 57.11 | I | O |
| ATOM | 1623 | NE2 | GLN | I | 3 | 10.991 | 29.310 | 16.878 | 1.00 | 53.43 | I | N |
| ATOM | 1624 | C | GLN | I | 3 | 13.335 | 28.024 | 21.756 | 1.00 | 29.16 | I | C |
| ATOM | 1625 | O | GLN | I | 3 | 12.432 | 28.359 | 22.536 | 1.00 | 27.84 | I | O |
| ATOM | 1626 | N | LEU | I | 4 | 14.627 | 28.202 | 22.005 | 1.00 | 24.53 | I | N |
| ATOM | 1627 | CA | LEU | I | 4 | 15.130 | 28.845 | 23.221 | 1.00 | 21.56 | I | C |
| ATOM | 1628 | CB | LEU | I | 4 | 15.637 | 27.807 | 24.224 | 1.00 | 19.57 | I | C |
| ATOM | 1629 | CG | LEU | I | 4 | 14.624 | 26.855 | 24.863 | 1.00 | 18.07 | I | C |
| ATOM | 1630 | CD1 | LEU | I | 4 | 15.341 | 26.056 | 25.912 | 1.00 | 20.92 | I | C |
| ATOM | 1631 | CD2 | LEU | I | 4 | 13.465 | 27.617 | 25.485 | 1.00 | 18.54 | I | C |
| ATOM | 1632 | C | LEU | I | 4 | 16.263 | 29.750 | 22.767 | 1.00 | 20.27 | I | C |
| ATOM | 1633 | O | LEU | I | 4 | 17.215 | 29.289 | 22.135 | 1.00 | 20.76 | I | O |
| ATOM | 1634 | N | VAL | I | 5 | 16.087 | 31.051 | 22.988 | 1.00 | 23.80 | I | N |
| ATOM | 1635 | CA | VAL | I | 5 | 17.056 | 32.070 | 22.583 | 1.00 | 23.96 | I | C |
| ATOM | 1636 | CB | VAL | I | 5 | 16.442 | 33.059 | 21.571 | 1.00 | 28.37 | I | C |
| ATOM | 1637 | CG1 | VAL | I | 5 | 17.436 | 34.184 | 21.236 | 1.00 | 19.85 | I | C |
| ATOM | 1638 | CG2 | VAL | I | 5 | 16.022 | 32.316 | 20.297 | 1.00 | 21.33 | I | C |
| ATOM | 1639 | C | VAL | I | 5 | 17.536 | 32.854 | 23.792 | 1.00 | 31.41 | I | C |
| ATOM | 1640 | O | VAL | I | 5 | 16.751 | 33.539 | 24.458 | 1.00 | 36.98 | I | O |
| ATOM | 1641 | N | GLU | I | 6 | 18.835 | 32.768 | 24.050 | 1.00 | 29.40 | I | N |
| ATOM | 1642 | CA | GLU | I | 6 | 19.447 | 33.439 | 25.186 | 1.00 | 26.13 | I | C |
| ATOM | 1643 | CB | GLU | I | 6 | 20.466 | 32.521 | 25.810 | 1.00 | 26.39 | I | C |
| ATOM | 1644 | CG | GLU | I | 6 | 19.955 | 31.139 | 25.904 | 1.00 | 32.68 | I | C |
| ATOM | 1645 | CD | GLU | I | 6 | 20.950 | 30.219 | 26.490 | 1.00 | 35.43 | I | C |
| ATOM | 1646 | OE1 | GLU | I | 6 | 21.668 | 30.663 | 27.410 | 1.00 | 45.42 | I | O |
| ATOM | 1647 | OE2 | GLU | I | 6 | 20.998 | 29.054 | 26.054 | 1.00 | 29.55 | I | O |
| ATOM | 1648 | C | GLU | I | 6 | 20.129 | 34.730 | 24.818 | 1.00 | 22.31 | I | C |
| ATOM | 1649 | O | GLU | I | 6 | 20.592 | 34.903 | 23.694 | 1.00 | 30.14 | I | O |
| ATOM | 1650 | N | SER | I | 7 | 20.168 | 35.643 | 25.774 | 1.00 | 20.69 | I | N |
| ATOM | 1651 | CA | SER | I | 7 | 20.825 | 36.924 | 25.584 | 1.00 | 18.76 | I | C |
| ATOM | 1652 | CB | SER | I | 7 | 19.868 | 37.921 | 24.939 | 1.00 | 17.88 | I | C |
| ATOM | 1653 | OG | SER | I | 7 | 18.764 | 38.159 | 25.787 | 1.00 | 21.59 | I | O |
| ATOM | 1654 | C | SER | I | 7 | 21.271 | 37.433 | 26.950 | 1.00 | 22.17 | I | C |
| ATOM | 1655 | O | SER | I | 7 | 20.855 | 36.905 | 27.999 | 1.00 | 14.08 | I | O |
| ATOM | 1656 | N | GLY | I | 8 | 22.124 | 38.453 | 26.936 | 1.00 | 23.92 | I | N |
| ATOM | 1657 | CA | GLY | I | 8 | 22.594 | 39.023 | 28.183 | 1.00 | 25.51 | I | C |
| ATOM | 1658 | C | GLY | I | 8 | 24.053 | 38.749 | 28.452 | 1.00 | 26.13 | I | C |
| ATOM | 1659 | O | GLY | I | 8 | 24.643 | 39.342 | 29.353 | 1.00 | 32.93 | I | O |
| ATOM | 1660 | N | GLY | I | 9 | 24.631 | 37.845 | 27.673 | 1.00 | 27.44 | I | N |
| ATOM | 1661 | CA | GLY | I | 9 | 26.030 | 37.515 | 27.839 | 1.00 | 25.03 | I | C |
| ATOM | 1662 | C | GLY | I | 9 | 26.913 | 38.685 | 27.485 | 1.00 | 26.42 | I | C |
| ATOM | 1663 | O | GLY | I | 9 | 26.474 | 39.622 | 26.818 | 1.00 | 26.45 | I | O |
| ATOM | 1664 | N | GLY | I | 10 | 28.159 | 38.631 | 27.944 | 1.00 | 25.98 | I | N |
| ATOM | 1665 | CA | GLY | I | 10 | 29.096 | 39.698 | 27.664 | 1.00 | 29.37 | I | C |
| ATOM | 1666 | C | GLY | I | 10 | 30.141 | 39.823 | 28.747 | 1.00 | 28.48 | I | C |
| ATOM | 1667 | O | GLY | I | 10 | 30.195 | 39.006 | 29.662 | 1.00 | 31.77 | I | O |
| ATOM | 1668 | N | LEU | I | 11 | 30.963 | 40.860 | 28.635 | 1.00 | 28.50 | I | N |
| ATOM | 1669 | CA | LEU | I | 11 | 32.040 | 41.138 | 29.576 | 1.00 | 26.46 | I | C |
| ATOM | 1670 | CB | LEU | I | 11 | 33.060 | 42.052 | 28.890 | 1.00 | 27.78 | I | C |
| ATOM | 1671 | CG | LEU | I | 11 | 34.215 | 42.597 | 29.735 | 1.00 | 34.24 | I | C |
| ATOM | 1672 | CD1 | LEU | I | 11 | 35.012 | 41.435 | 30.370 | 1.00 | 29.89 | 1 | C |
| ATOM | 1673 | CD2 | LEU | I | 11 | 35.094 | 43.476 | 28.859 | 1.00 | 28.65 | I | C |
| ATOM | 1674 | C | LEU | I | 11 | 31.580 | 41.765 | 30.901 | 1.00 | 25.67 | I | C |
| ATOM | 1675 | O | LEU | I | 11 | 30.883 | 42.784 | 30.911 | 1.00 | 30.87 | I | O |
| ATOM | 1676 | N | VAL | I | 12 | 31.990 | 41.160 | 32.015 | 1.00 | 21.60 | I | N |
| ATOM | 1677 | CA | VAL | I | 12 | 31.655 | 41.659 | 33.348 | 1.00 | 21.79 | I | C |
| ATOM | 1678 | CB | VAL | I | 12 | 30.625 | 40.754 | 34.073 | 1.00 | 23.55 | I | C |
| ATOM | 1679 | CG1 | VAL | I | 12 | 30.170 | 41.417 | 35.354 | 1.00 | 25.81 | I | C |
| ATOM | 1680 | CG2 | VAL | I | 12 | 29.435 | 40.476 | 33.196 | 1.00 | 28.55 | I | C |
| ATOM | 1681 | C | VAL | I | 12 | 32.897 | 41.731 | 34.245 | 1.00 | 22.41 | I | C |
| ATOM | 1682 | O | VAL | I | 12 | 33.732 | 40.823 | 34.238 | 1.00 | 23.26 | I | O |
| ATOM | 1683 | N | GLN | I | 13 | 33.008 | 42.805 | 35.024 | 1.00 | 23.37 | I | N |
| ATOM | 1684 | CA | GLN | I | 13 | 34.130 | 42.965 | 35.949 | 1.00 | 23.01 | I | C |
| ATOM | 1685 | CB | GLN | I | 13 | 34.214 | 44.405 | 36.483 | 1.00 | 28.51 | I | C |
| ATOM | 1686 | CG | GLN | I | 13 | 34.237 | 45.521 | 35.449 | 1.00 | 32.27 | I | C |
| ATOM | 1687 | CD | GLN | I | 13 | 35.510 | 45.560 | 34.641 | 1.00 | 33.64 | I | C |
| ATOM | 1688 | OE1 | GLN | I | 13 | 36.610 | 45.567 | 35.187 | 1.00 | 43.28 | I | O |
| ATOM | 1689 | NE2 | GLN | I | 13 | 35.367 | 45.599 | 33.326 | 1.00 | 39.43 | I | N |
| ATOM | 1690 | C | GLN | I | 13 | 33.910 | 42.028 | 37.138 | 1.00 | 26.40 | I | C |
| ATOM | 1691 | O | GLN | I | 13 | 32.762 | 41.730 | 37.498 | 1.00 | 24.25 | I | O |
| ATOM | 1692 | N | PRO | I | 14 | 35.005 | 41.549 | 37.762 | 1.00 | 21.94 | I | N |
| ATOM | 1693 | CD | PRO | I | 14 | 36.401 | 41.738 | 37.345 | 1.00 | 22.69 | I | C |
| ATOM | 1694 | CA | PRO | I | 14 | 34.927 | 40.651 | 38.918 | 1.00 | 23.33 | I | C |
| ATOM | 1695 | CB | PRO | I | 14 | 36.390 | 40.467 | 39.299 | 1.00 | 23.24 | I | C |
| ATOM | 1696 | CG | PRO | I | 14 | 37.081 | 40.550 | 37.976 | 1.00 | 24.43 | I | C |
| ATOM | 1697 | C | PRO | I | 14 | 34.167 | 41.350 | 40.038 | 1.00 | 26.68 | I | C |
| ATOM | 1698 | O | PRO | I | 14 | 34.451 | 42.513 | 40.349 | 1.00 | 28.80 | I | O |
| ATOM | 1699 | N | GLY | I | 15 | 33.182 | 40.655 | 40.608 | 1.00 | 25.46 | I | N |
| ATOM | 1700 | CA | GLY | I | 15 | 32.397 | 41.229 | 41.685 | 1.00 | 21.25 | I | C |
| ATOM | 1701 | C | GLY | I | 15 | 31.109 | 41.804 | 41.147 | 1.00 | 26.59 | I | C |
| ATOM | 1702 | O | GLY | I | 15 | 30.206 | 42.161 | 41.916 | 1.00 | 25.56 | I | O |
| ATOM | 1703 | N | GLY | I | 16 | 31.028 | 41.907 | 39.822 | 1.00 | 14.13 | I | N |
| ATOM | 1704 | CA | GLY | I | 16 | 29.826 | 42.420 | 39.195 | 1.00 | 23.58 | I | C |
| ATOM | 1705 | C | GLY | I | 16 | 28.736 | 41.360 | 39.111 | 1.00 | 28.61 | I | C |
| ATOM | 1706 | O | GLY | I | 16 | 28.840 | 40.286 | 39.717 | 1.00 | 26.93 | I | O |
| ATOM | 1707 | N | SER | I | 17 | 27.700 | 41.638 | 38.327 | 1.00 | 33.32 | I | N |
| ATOM | 1708 | CA | SER | I | 17 | 26.594 | 40.701 | 38.189 | 1.00 | 35.55 | I | C |
| ATOM | 1709 | CB | SER | I | 17 | 25.394 | 41.183 | 39.001 | 1.00 | 32.56 | I | C |
| ATOM | 1710 | OG | SER | I | 17 | 25.018 | 42.483 | 38.595 | 1.00 | 44.85 | I | O |
| ATOM | 1711 | C | SER | I | 17 | 26.183 | 40.468 | 36.747 | 1.00 | 37.75 | I | C |
| ATOM | 1712 | O | SER | I | 17 | 26.452 | 41.288 | 35.864 | 1.00 | 38.47 | I | O |
| ATOM | 1713 | N | LEU | I | 18 | 25.532 | 39.332 | 36.520 | 1.00 | 37.20 | I | N |
| ATOM | 1714 | CA | LEU | I | 18 | 25.065 | 38.965 | 35.198 | 1.00 | 35.26 | I | C |
| ATOM | 1715 | CB | LEU | I | 18 | 25.786 | 37.697 | 34.714 | 1.00 | 30.08 | I | C |
| ATOM | 1716 | CG | LEU | I | 18 | 25.615 | 36.354 | 35.448 | 1.00 | 33.93 | I | C |
| ATOM | 1717 | CD1 | LEU | I | 18 | 24.223 | 35.756 | 35.249 | 1.00 | 26.73 | I | C |
| ATOM | 1718 | CD2 | LEU | I | 18 | 26.646 | 35.357 | 34.928 | 1.00 | 35.44 | I | C |
| ATOM | 1719 | C | LEU | I | 18 | 23.557 | 38.743 | 35.238 | 1.00 | 34.94 | I | C |
| ATOM | 1720 | O | LEU | I | 18 | 23.016 | 38.267 | 36.234 | 1.00 | 32.86 | I | O |
| ATOM | 1721 | N | ARG | I | 19 | 22.882 | 39.109 | 34.156 | 1.00 | 37.91 | I | N |
| ATOM | 1722 | CA | ARG | I | 19 | 21.438 | 38.930 | 34.032 | 1.00 | 32.12 | I | C |
| ATOM | 1723 | CB | ARG | I | 19 | 20.705 | 40.268 | 34.159 | 1.00 | 45.56 | I | C |
| ATOM | 1724 | CG | ARG | I | 19 | 20.266 | 40.657 | 35.580 | 1.00 | 61.36 | I | C |
| ATOM | 1725 | CD | ARG | I | 19 | 21.425 | 40.806 | 36.608 | 1.00 | 66.95 | I | C |
| ATOM | 1726 | NE | ARG | I | 19 | 22.378 | 41.892 | 36.336 | 1.00 | 70.47 | I | N |
| ATOM | 1727 | CZ | ARG | I | 19 | 22.169 | 43.177 | 36.621 | 1.00 | 72.62 | I | C |
| ATOM | 1728 | NH1 | ARG | I | 19 | 21.027 | 43.568 | 37.185 | 1.00 | 72.62 | I | N |
| ATOM | 1729 | NH2 | ARG | I | 19 | 23.124 | 44.070 | 36.374 | 1.00 | 72.57 | I | N |
| ATOM | 1730 | C | ARG | I | 19 | 21.184 | 38.342 | 32.652 | 1.00 | 27.56 | I | C |
| ATOM | 1731 | O | ARG | I | 19 | 21.205 | 39.056 | 31.649 | 1.00 | 23.55 | I | O |
| ATOM | 1732 | N | LEU | I | 20 | 20.999 | 37.029 | 32.608 | 1.00 | 19.30 | I | N |
| ATOM | 1733 | CA | LEU | I | 20 | 20.754 | 36.308 | 31.354 | 1.00 | 23.13 | I | C |
| ATOM | 1734 | CB | LEU | I | 20 | 21.476 | 34.952 | 31.380 | 1.00 | 18.94 | I | C |
| ATOM | 1735 | CG | LEU | I | 20 | 22.961 | 34.979 | 31.746 | 1.00 | 13.63 | I | C |
| ATOM | 1736 | CD1 | LEU | I | 20 | 23.524 | 33.596 | 31.531 | 1.00 | 18.06 | I | C |
| ATOM | 1737 | CD2 | LEU | I | 20 | 23.700 | 35.986 | 30.888 | 1.00 | 15.40 | I | C |
| ATOM | 1738 | C | LEU | I | 20 | 19.266 | 36.074 | 31.112 | 1.00 | 13.54 | I | C |
| ATOM | 1739 | O | LEU | I | 20 | 18.529 | 35.758 | 32.038 | 1.00 | 22.06 | I | O |
| ATOM | 1740 | N | SER | I | 21 | 18.837 | 36.213 | 29.867 | 1.00 | 18.60 | I | N |
| ATOM | 1741 | CA | SER | I | 21 | 17.435 | 36.015 | 29.472 | 1.00 | 22.13 | I | C |
| ATOM | 1742 | CB | SER | I | 21 | 16.950 | 37.195 | 28.622 | 1.00 | 18.45 | I | C |
| ATOM | 1743 | OG | SER | I | 21 | 17.095 | 38.452 | 29.263 | 1.00 | 39.38 | I | O |
| ATOM | 1744 | C | SER | I | 21 | 17.310 | 34.787 | 28.573 | 1.00 | 26.06 | I | C |
| ATOM | 1745 | O | SER | I | 21 | 18.204 | 34.514 | 27.765 | 1.00 | 24.43 | I | O |
| ATOM | 1746 | N | CYS | I | 22 | 16.186 | 34.086 | 28.665 | 1.00 | 24.55 | I | N |
| ATOM | 1747 | CA | CYS | I | 22 | 15.953 | 32.932 | 27.805 | 1.00 | 27.98 | I | C |
| ATOM | 1748 | C | CYS | I | 22 | 14.532 | 33.015 | 27.237 | 1.00 | 28.48 | I | C |
| ATOM | 1749 | O | CYS | I | 22 | 13.559 | 32.648 | 27.909 | 1.00 | 29.76 | I | O |
| ATOM | 1750 | CB | CYS | I | 22 | 16.132 | 31.642 | 28.583 | 1.00 | 32.37 | I | C |
| ATOM | 1751 | SG | CYS | I | 22 | 15.917 | 30.143 | 27.569 | 1.00 | 44.03 | I | S |
| ATOM | 1752 | N | ALA | I | 23 | 14.417 | 33.529 | 26.015 | 1.00 | 22.24 | I | N |
| ATOM | 1753 | CA | ALA | I | 23 | 13.115 | 33.687 | 25.358 | 1.00 | 23.42 | I | C |
| ATOM | 1754 | CB | ALA | I | 23 | 13.181 | 34.774 | 24.283 | 1.00 | 15.40 | I | C |
| ATOM | 1755 | C | ALA | I | 23 | 12.667 | 32.372 | 24.756 | 1.00 | 20.67 | I | C |
| ATOM | 1756 | O | ALA | I | 23 | 13.383 | 31.773 | 23.958 | 1.00 | 28.67 | I | O |
| ATOM | 1757 | N | ALA | I | 24 | 11.520 | 31.885 | 25.211 | 1.00 | 20.52 | I | N |
| ATOM | 1758 | CA | ALA | I | 24 | 10.973 | 30.622 | 24.719 | 1.00 | 19.15 | I | C |
| ATOM | 1759 | CB | ALA | I | 24 | 10.326 | 29.841 | 25.856 | 1.00 | 16.08 | I | C |
| ATOM | 1760 | C | ALA | I | 24 | 9.965 | 30.855 | 23.612 | 1.00 | 17.36 | I | C |
| ATOM | 1761 | O | ALA | I | 24 | 9.342 | 31.912 | 23.539 | 1.00 | 19.51 | I | O |
| ATOM | 1762 | N | SER | I | 25 | 9.820 | 29.847 | 22.758 | 1.00 | 25.55 | I | N |
| ATOM | 1763 | CA | SER | I | 25 | 8.889 | 29.862 | 21.628 | 1.00 | 21.59 | I | C |
| ATOM | 1764 | CB | SER | I | 25 | 9.618 | 30.325 | 20.377 | 1.00 | 22.72 | I | C |
| ATOM | 1765 | OG | SER | I | 25 | 9.304 | 31.655 | 20.084 | 1.00 | 30.42 | I | O |
| ATOM | 1766 | C | SER | I | 25 | 8.425 | 28.435 | 21.386 | 1.00 | 26.18 | I | C |
| ATOM | 1767 | O | SER | I | 25 | 9.209 | 27.489 | 21.557 | 1.00 | 24.77 | I | O |
| ATOM | 1768 | N | GLY | I | 26 | 7.168 | 28.282 | 20.970 | 1.00 | 26.28 | I | N |
| ATOM | 1769 | CA | GLY | I | 26 | 6.629 | 26.961 | 20.677 | 1.00 | 21.98 | I | C |
| ATOM | 1770 | C | GLY | I | 26 | 6.128 | 26.134 | 21.849 | 1.00 | 21.90 | I | C |
| ATOM | 1771 | O | GLY | I | 26 | 5.834 | 24.948 | 21.701 | 1.00 | 26.12 | I | O |
| ATOM | 1772 | N | PHE | I | 27 | 6.059 | 26.746 | 23.020 | 1.00 | 15.81 | I | N |
| ATOM | 1773 | CA | PHE | I | 27 | 5.579 | 26.076 | 24.219 | 1.00 | 15.00 | I | C |
| ATOM | 1774 | CB | PHE | I | 27 | 6.524 | 24.944 | 24.626 | 1.00 | 15.86 | I | C |
| ATOM | 1775 | CG | PHE | I | 27 | 7.798 | 25.418 | 25.275 | 1.00 | 20.99 | I | C |
| ATOM | 1776 | CD1 | PHE | I | 27 | 8.864 | 25.858 | 24.504 | 1.00 | 17.22 | I | C |
| ATOM | 1777 | CD2 | PHE | I | 27 | 7.926 | 25.424 | 26.660 | 1.00 | 19.41 | I | C |
| ATOM | 1778 | CE1 | PHE | I | 27 | 10.034 | 26.295 | 25.101 | 1.00 | 20.12 | I | C |
| ATOM | 1779 | CE2 | PHE | I | 27 | 9.102 | 25.862 | 27.269 | 1.00 | 16.64 | I | C |
| ATOM | 1780 | CZ | PHE | I | 27 | 10.156 | 26.297 | 26.486 | 1.00 | 19.02 | I | C |
| ATOM | 1781 | C | PHE | I | 27 | 5.496 | 27.154 | 25.304 | 1.00 | 20.13 | I | C |
| ATOM | 1782 | O | PHE | I | 27 | 5.902 | 28.294 | 25.075 | 1.00 | 30.89 | I | O |
| ATOM | 1783 | N | ASN | I | 28 | 4.940 | 26.826 | 26.463 | 1.00 | 24.47 | I | N |
| ATOM | 1784 | CA | ASN | I | 28 | 4.826 | 27.814 | 27.534 | 1.00 | 26.58 | I | C |
| ATOM | 1785 | CB | ASN | I | 28 | 3.364 | 27.987 | 27.932 | 1.00 | 28.93 | I | C |
| ATOM | 1786 | CG | ASN | I | 28 | 2.522 | 28.540 | 26.792 | 1.00 | 39.06 | I | C |
| ATOM | 1787 | OD1 | ASN | I | 28 | 2.799 | 29.627 | 26.271 | 1.00 | 44.31 | I | O |
| ATOM | 1788 | ND2 | ASN | I | 28 | 1.511 | 27.782 | 26.376 | 1.00 | 37.74 | I | N |
| ATOM | 1789 | C | ASN | I | 28 | 5.677 | 27.415 | 28.726 | 1.00 | 24.64 | I | C |
| ATOM | 1790 | O | ASN | I | 28 | 5.478 | 26.346 | 29.291 | 1.00 | 22.74 | I | O |
| ATOM | 1791 | N | ILE | I | 29 | 6.633 | 28.274 | 29.090 | 1.00 | 23.68 | I | N |
| ATOM | 1792 | CA | ILE | I | 29 | 7.535 | 27.991 | 30.200 | 1.00 | 26.37 | I | C |
| ATOM | 1793 | CB | ILE | I | 29 | 8.527 | 29.169 | 30.501 | 1.00 | 29.61 | I | C |
| ATOM | 1794 | CG2 | ILE | I | 29 | 9.623 | 29.267 | 29.454 | 1.00 | 26.93 | I | C |
| ATOM | 1795 | CG1 | ILE | I | 29 | 7.774 | 30.487 | 30.614 | 1.00 | 37.63 | I | C |
| ATOM | 1796 | CD1 | ILE | I | 29 | 7.333 | 30.812 | 32.019 | 1.00 | 45.03 | I | C |
| ATOM | 1797 | C | ILE | I | 29 | 6.755 | 27.679 | 31.452 | 1.00 | 23.75 | I | C |
| ATOM | 1798 | O | ILE | I | 29 | 7.191 | 26.887 | 32.284 | 1.00 | 25.58 | I | O |
| ATOM | 1799 | N | LYS | I | 30 | 5.576 | 28.280 | 31.551 | 1.00 | 29.69 | I | N |
| ATOM | 1800 | CA | LYS | I | 30 | 4.701 | 28.112 | 32.706 | 1.00 | 32.40 | I | C |
| ATOM | 1801 | CB | LYS | I | 30 | 3.389 | 28.875 | 32.468 | 1.00 | 40.21 | I | C |
| ATOM | 1802 | CG | LYS | I | 30 | 2.496 | 29.008 | 33.694 | 1.00 | 48.82 | I | C |
| ATOM | 1803 | CD | LYS | I | 30 | 1.127 | 29.618 | 33.374 | 1.00 | 51.45 | I | C |
| ATOM | 1804 | CE | LYS | I | 30 | 0.395 | 30.042 | 34.668 | 1.00 | 53.71 | I | C |
| ATOM | 1805 | NZ | LYS | I | 30 | 0.392 | 28.994 | 35.751 | 1.00 | 48.95 | I | N |
| ATOM | 1806 | C | LYS | I | 30 | 4.399 | 26.635 | 33.019 | 1.00 | 32.53 | I | C |
| ATOM | 1807 | O | LYS | I | 30 | 4.216 | 26.271 | 34.176 | 1.00 | 32.88 | I | O |
| ATOM | 1808 | N | GLU | I | 31 | 4.397 | 25.792 | 31.991 | 1.00 | 32.46 | I | N |
| ATOM | 1809 | CA | GLU | I | 31 | 4.092 | 24.366 | 32.128 | 1.00 | 36.25 | I | C |
| ATOM | 1810 | CB | GLU | I | 31 | 3.235 | 23.903 | 30.938 | 1.00 | 40.43 | I | C |
| ATOM | 1811 | CG | GLU | I | 31 | 1.950 | 24.692 | 30.666 | 1.00 | 46.68 | I | C |
| ATOM | 1812 | CD | GLU | I | 31 | 1.411 | 24.484 | 29.241 | 1.00 | 50.18 | I | C |
| ATOM | 1813 | OE1 | GLU | I | 31 | 1.601 | 23.385 | 28.663 | 1.00 | 49.80 | I | O |
| ATOM | 1814 | OE2 | GLU | I | 31 | 0.802 | 25.434 | 28.696 | 1.00 | 51.17 | I | O |
| ATOM | 1815 | C | GLU | I | 31 | 5.292 | 23.419 | 32.192 | 1.00 | 33.17 | I | C |
| ATOM | 1816 | O | GLU | I | 31 | 5.105 | 22.204 | 32.246 | 1.00 | 32.99 | I | O |
| ATOM | 1817 | N | TYR | I | 32 | 6.513 | 23.940 | 32.172 | 1.00 | 30.55 | I | N |
| ATOM | 1818 | CA | TYR | I | 32 | 7.686 | 23.062 | 32.170 | 1.00 | 25.13 | I | C |
| ATOM | 1819 | CB | TYR | I | 32 | 8.382 | 23.126 | 30.804 | 1.00 | 22.13 | I | C |
| ATOM | 1820 | CG | TYR | I | 32 | 7.693 | 22.389 | 29.681 | 1.00 | 26.95 | I | C |
| ATOM | 1821 | CD1 | TYR | I | 32 | 6.639 | 22.974 | 28.975 | 1.00 | 24.32 | I | C |
| ATOM | 1822 | CE1 | TYR | I | 32 | 6.020 | 22.309 | 27.926 | 1.00 | 22.13 | I | C |
| ATOM | 1823 | CD2 | TYR | I | 32 | 8.110 | 21.106 | 29.302 | 1.00 | 28.33 | I | C |
| ATOM | 1824 | CE2 | TYR | I | 32 | 7.492 | 20.428 | 28.247 | 1.00 | 26.27 | I | C |
| ATOM | 1825 | CZ | TYR | I | 32 | 6.450 | 21.040 | 27.566 | 1.00 | 26.11 | I | C |
| ATOM | 1826 | OH | TYR | I | 32 | 5.851 | 20.398 | 26.512 | 1.00 | 28.17 | I | O |
| ATOM | 1827 | C | TYR | I | 32 | 8.741 | 23.403 | 33.192 | 1.00 | 21.60 | I | C |
| ATOM | 1828 | O | TYR | I | 32 | 8.840 | 24.547 | 33.626 | 1.00 | 26.04 | I | O |
| ATOM | 1829 | N | TYR | I | 33 | 9.542 | 22.407 | 33.561 | 1.00 | 17.19 | I | N |
| ATOM | 1830 | CA | TYR | I | 33 | 10.672 | 22.646 | 34.453 | 1.00 | 18.64 | I | C |
| ATOM | 1831 | CB | TYR | I | 33 | 11.201 | 21.348 | 35.068 | 1.00 | 16.14 | I | C |
| ATOM | 1832 | CG | TYR | I | 33 | 10.339 | 20.796 | 36.166 | 1.00 | 18.81 | I | C |
| ATOM | 1833 | CD1 | TYR | I | 33 | 9.148 | 20.118 | 35.878 | 1.00 | 18.66 | I | C |
| ATOM | 1834 | CE1 | TYR | I | 33 | 8.333 | 19.632 | 36.911 | 1.00 | 22.44 | I | C |
| ATOM | 1835 | CD2 | TYR | I | 33 | 10.696 | 20.968 | 37.506 | 1.00 | 18.20 | I | C |
| ATOM | 1836 | CE2 | TYR | I | 33 | 9.897 | 20.486 | 38.532 | 1.00 | 20.26 | I | C |
| ATOM | 1837 | CZ | TYR | I | 33 | 8.720 | 19.826 | 38.232 | 1.00 | 19.53 | I | C |
| ATOM | 1838 | OH | TYR | I | 33 | 7.925 | 19.382 | 39.257 | 1.00 | 26.96 | I | O |
| ATOM | 1839 | C | TYR | I | 33 | 11.766 | 23.234 | 33.550 | 1.00 | 22.19 | I | C |
| ATOM | 1840 | O | TYR | I | 33 | 12.084 | 22.654 | 32.496 | 1.00 | 21.81 | I | O |
| ATOM | 1841 | N | MET | I | 34 | 12.277 | 24.404 | 33.932 | 1.00 | 15.88 | I | N |
| ATOM | 1842 | CA | MET | I | 34 | 13.342 | 25.097 | 33.210 | 1.00 | 19.35 | I | C |
| ATOM | 1843 | CB | MET | I | 34 | 12.902 | 26.533 | 32.902 | 1.00 | 24.92 | I | C |
| ATOM | 1844 | CG | MET | I | 34 | 11.731 | 26.622 | 31.920 | 1.00 | 26.58 | I | C |
| ATOM | 1845 | SD | MET | I | 34 | 12.095 | 25.757 | 30.367 | 1.00 | 28.46 | I | S |
| ATOM | 1846 | CE | MET | I | 34 | 12.927 | 26.958 | 29.558 | 1.00 | 17.04 | I | C |
| ATOM | 1847 | C | MET | I | 34 | 14.647 | 25.093 | 34.043 | 1.00 | 22.48 | I | C |
| ATOM | 1848 | O | MET | I | 34 | 14.621 | 25.291 | 35.275 | 1.00 | 18.43 | I | O |
| ATOM | 1849 | N | HIS | I | 35 | 15.778 | 24.843 | 33.380 | 1.00 | 20.06 | I | N |
| ATOM | 1850 | CA | HIS | I | 35 | 17.088 | 24.790 | 34.059 | 1.00 | 15.88 | I | C |
| ATOM | 1851 | CB | HIS | I | 35 | 17.690 | 23.376 | 34.016 | 1.00 | 10.04 | I | C |
| ATOM | 1852 | CG | HIS | I | 35 | 16.800 | 22.296 | 34.551 | 1.00 | 15.80 | I | C |
| ATOM | 1853 | CD2 | HIS | I | 35 | 15.559 | 21.891 | 34.183 | 1.00 | 10.65 | I | C |
| ATOM | 1854 | ND1 | HIS | I | 35 | 17.199 | 21.441 | 35.554 | 1.00 | 9.91 | I | N |
| ATOM | 1855 | CE1 | HIS | I | 35 | 16.245 | 20.556 | 35.784 | 1.00 | 14.15 | I | C |
| ATOM | 1856 | NE2 | HIS | I | 35 | 15.239 | 20.807 | 34.966 | 1.00 | 12.39 | I | N |
| ATOM | 1857 | C | HIS | I | 35 | 18.125 | 25.688 | 33.409 | 1.00 | 18.29 | I | C |
| ATOM | 1858 | O | HIS | I | 35 | 17.941 | 26.208 | 32.310 | 1.00 | 21.77 | I | O |
| ATOM | 1859 | N | TRP | I | 36 | 19.246 | 25.846 | 34.097 | 1.00 | 19.62 | I | N |
| ATOM | 1860 | CA | TRP | I | 36 | 20.354 | 26.603 | 33.553 | 1.00 | 15.55 | I | C |
| ATOM | 1861 | CB | TRP | I | 36 | 20.525 | 27.936 | 34.244 | 1.00 | 13.92 | I | C |
| ATOM | 1862 | CG | TRP | I | 36 | 19.545 | 28.938 | 33.783 | 1.00 | 14.44 | I | C |
| ATOM | 1863 | CD2 | TRP | I | 36 | 19.699 | 29.838 | 32.679 | 1.00 | 14.72 | I | C |
| ATOM | 1864 | CE2 | TRP | I | 36 | 18.571 | 30.691 | 32.675 | 1.00 | 13.44 | I | C |
| ATOM | 1865 | CE3 | TRP | I | 36 | 20.685 | 30.014 | 31.703 | 1.00 | 13.60 | I | C |
| ATOM | 1866 | CD1 | TRP | I | 36 | 18.353 | 29.258 | 34.378 | 1.00 | 12.89 | I | C |
| ATOM | 1867 | NE1 | TRP | I | 36 | 17.767 | 30.317 | 33.721 | 1.00 | 19.00 | I | N |
| ATOM | 1868 | CZ2 | TRP | I | 36 | 18.403 | 31.701 | 31.730 | 1.00 | 15.97 | I | C |
| ATOM | 1869 | CZ3 | TRP | I | 36 | 20.520 | 31.025 | 30.753 | 1.00 | 16.69 | I | C |
| ATOM | 1870 | CH2 | TRP | I | 36 | 19.389 | 31.855 | 30.774 | 1.00 | 17.79 | I | C |
| ATOM | 1871 | C | TRP | I | 36 | 21.552 | 25.729 | 33.779 | 1.00 | 15.29 | I | C |
| ATOM | 1872 | O | TRP | I | 36 | 21.695 | 25.152 | 34.843 | 1.00 | 23.16 | I | O |
| ATOM | 1873 | N | VAL | I | 37 | 22.357 | 25.569 | 32.733 | 1.00 | 19.03 | I | N |
| ATOM | 1874 | CA | VAL | I | 37 | 23.566 | 24.758 | 32.747 | 1.00 | 11.92 | I | C |
| ATOM | 1875 | CB | VAL | I | 37 | 23.375 | 23.493 | 31.911 | 1.00 | 16.23 | I | C |
| ATOM | 1876 | CG1 | VAL | I | 37 | 24.717 | 22.797 | 31.641 | 1.00 | 14.20 | I | C |
| ATOM | 1877 | CG2 | VAL | I | 37 | 22.423 | 22.545 | 32.633 | 1.00 | 13.93 | I | C |
| ATOM | 1878 | C | VAL | I | 37 | 24.689 | 25.607 | 32.157 | 1.00 | 18.58 | I | C |
| ATOM | 1879 | O | VAL | I | 37 | 24.432 | 26.496 | 31.359 | 1.00 | 19.68 | I | O |
| ATOM | 1880 | N | ARG | I | 38 | 25.922 | 25.373 | 32.599 | 1.00 | 20.71 | I | N |
| ATOM | 1881 | CA | ARG | I | 38 | 27.066 | 26.139 | 32.112 | 1.00 | 14.17 | I | C |
| ATOM | 1882 | CB | ARG | I | 38 | 27.441 | 27.229 | 33.102 | 1.00 | 17.20 | I | C |
| ATOM | 1883 | CG | ARG | I | 38 | 28.155 | 26.727 | 34.354 | 1.00 | 16.52 | I | C |
| ATOM | 1884 | CD | ARG | I | 38 | 28.624 | 27.908 | 35.180 | 1.00 | 20.21 | I | C |
| ATOM | 1885 | NE | ARG | I | 38 | 29.391 | 27.476 | 36.337 | 1.00 | 29.12 | I | N |
| ATOM | 1886 | CZ | ARG | I | 38 | 29.846 | 28.290 | 37.280 | 1.00 | 28.96 | I | C |
| ATOM | 1887 | NH1 | ARG | I | 38 | 29.615 | 29.594 | 37.210 | 1.00 | 25.33 | I | N |
| ATOM | 1888 | NH2 | ARG | I | 38 | 30.528 | 27.793 | 38.296 | 1.00 | 25.32 | I | N |
| ATOM | 1889 | C | ARG | I | 38 | 28.298 | 25.298 | 31.836 | 1.00 | 10.28 | I | C |
| ATOM | 1890 | O | ARG | I | 38 | 28.472 | 24.188 | 32.341 | 1.00 | 15.37 | I | O |
| ATOM | 1891 | N | GLN | I | 39 | 29.178 | 25.866 | 31.051 | 1.00 | 13.20 | I | N |
| ATOM | 1892 | CA | GLN | I | 39 | 30.391 | 25.182 | 30.666 | 1.00 | 18.36 | I | C |
| ATOM | 1893 | CB | GLN | I | 39 | 30.188 | 24.459 | 29.330 | 1.00 | 15.39 | I | C |
| ATOM | 1894 | CG | GLN | I | 39 | 31.351 | 23.597 | 28.906 | 1.00 | 13.39 | I | C |
| ATOM | 1895 | CD | GLN | I | 39 | 30.964 | 22.560 | 27.847 | 1.00 | 20.38 | I | C |
| ATOM | 1896 | OE1 | GLN | I | 39 | 30.330 | 22.889 | 26.850 | 1.00 | 22.01 | I | O |
| ATOM | 1897 | NE2 | GLN | I | 39 | 31.361 | 21.304 | 28.063 | 1.00 | 13.72 | I | N |
| ATOM | 1898 | C | GLN | I | 39 | 31.507 | 26.194 | 30.518 | 1.00 | 20.45 | I | C |
| ATOM | 1899 | O | GLN | I | 39 | 31.448 | 27.069 | 29.655 | 1.00 | 17.60 | I | O |
| ATOM | 1900 | N | ALA | I | 40 | 32.467 | 26.125 | 31.436 | 1.00 | 20.92 | I | N |
| ATOM | 1901 | CA | ALA | I | 40 | 33.638 | 26.993 | 31.407 | 1.00 | 24.19 | I | C |
| ATOM | 1902 | CB | ALA | I | 40 | 34.406 | 26.875 | 32.756 | 1.00 | 11.61 | I | C |
| ATOM | 1903 | C | ALA | I | 40 | 34.520 | 26.524 | 30.222 | 1.00 | 23.27 | I | C |
| ATOM | 1904 | O | ALA | I | 40 | 34.491 | 25.345 | 29.829 | 1.00 | 20.46 | I | O |
| ATOM | 1905 | N | PRO | I | 41 | 35.303 | 27.443 | 29.642 | 1.00 | 25.02 | I | N |
| ATOM | 1906 | CD | PRO | I | 41 | 35.480 | 28.837 | 30.104 | 1.00 | 27.13 | I | C |
| ATOM | 1907 | CA | PRO | I | 41 | 36.192 | 27.149 | 28.510 | 1.00 | 26.64 | I | C |
| ATOM | 1908 | CB | PRO | I | 41 | 37.107 | 28.383 | 28.477 | 1.00 | 24.76 | I | C |
| ATOM | 1909 | CG | PRO | I | 41 | 36.202 | 29.490 | 28.930 | 1.00 | 19.18 | I | C |
| ATOM | 1910 | C | PRO | I | 41 | 37.010 | 25.863 | 28.700 | 1.00 | 28.87 | I | C |
| ATOM | 1911 | O | PRO | I | 41 | 37.737 | 25.725 | 29.680 | 1.00 | 30.78 | I | O |
| ATOM | 1912 | N | GLY | I | 42 | 36.855 | 24.913 | 27.778 | 1.00 | 31.93 | I | N |
| ATOM | 1913 | CA | GLY | I | 42 | 37.596 | 23.663 | 27.843 | 1.00 | 24.75 | I | C |
| ATOM | 1914 | C | GLY | I | 42 | 37.301 | 22.757 | 29.030 | 1.00 | 29.28 | I | C |
| ATOM | 1915 | O | GLY | I | 42 | 38.075 | 21.834 | 29.315 | 1.00 | 28.78 | I | O |
| ATOM | 1916 | N | LYS | I | 43 | 36.173 | 22.979 | 29.699 | 1.00 | 22.54 | I | N |
| ATOM | 1917 | CA | LYS | I | 43 | 35.809 | 22.176 | 30.857 | 1.00 | 19.73 | I | C |
| ATOM | 1918 | CB | LYS | I | 43 | 35.601 | 23.094 | 32.065 | 1.00 | 27.07 | I | C |
| ATOM | 1919 | CG | LYS | I | 43 | 36.859 | 23.806 | 32.583 | 1.00 | 31.69 | I | C |
| ATOM | 1920 | CD | LYS | I | 43 | 37.899 | 22.817 | 33.083 | 1.00 | 34.04 | I | C |
| ATOM | 1921 | CE | LYS | I | 43 | 38.732 | 23.377 | 34.249 | 1.00 | 45.48 | I | C |
| ATOM | 1922 | NZ | LYS | I | 43 | 39.640 | 24.508 | 33.897 | 1.00 | 45.93 | I | N |
| ATOM | 1923 | C | LYS | I | 43 | 34.552 | 21.331 | 30.638 | 1.00 | 22.26 | I | C |
| ATOM | 1924 | O | LYS | I | 43 | 33.981 | 21.318 | 29.546 | 1.00 | 26.24 | I | O |
| ATOM | 1925 | N | GLY | I | 44 | 34.136 | 20.612 | 31.680 | 1.00 | 24.42 | I | N |
| ATOM | 1926 | CA | GLY | I | 44 | 32.935 | 19.792 | 31.615 | 1.00 | 23.62 | I | C |
| ATOM | 1927 | C | GLY | I | 44 | 31.683 | 20.638 | 31.828 | 1.00 | 25.03 | I | C |
| ATOM | 1928 | O | GLY | I | 44 | 31.780 | 21.858 | 31.919 | 1.00 | 25.03 | I | O |
| ATOM | 1929 | N | LEU | I | 45 | 30.514 | 19.997 | 31.869 | 1.00 | 20.87 | I | N |
| ATOM | 1930 | CA | LEU | I | 45 | 29.229 | 20.672 | 32.069 | 1.00 | 13.13 | I | C |
| ATOM | 1931 | CB | LEU | I | 45 | 28.115 | 19.873 | 31.359 | 1.00 | 13.52 | I | C |
| ATOM | 1932 | CG | LEU | I | 45 | 28.191 | 19.715 | 29.839 | 1.00 | 8.20 | I | C |
| ATOM | 1933 | CD1 | LEU | I | 45 | 27.170 | 18.717 | 29.356 | 1.00 | 12.32 | I | C |
| ATOM | 1934 | CD2 | LEU | I | 45 | 27.959 | 21.027 | 29.163 | 1.00 | 10.98 | I | C |
| ATOM | 1935 | C | LEU | I | 45 | 28.879 | 20.777 | 33.570 | 1.00 | 19.87 | I | C |
| ATOM | 1936 | O | LEU | I | 45 | 29.196 | 19.863 | 34.342 | 1.00 | 15.20 | I | O |
| ATOM | 1937 | N | GLU | I | 46 | 28.232 | 21.877 | 33.983 | 1.00 | 17.91 | I | N |
| ATOM | 1938 | CA | GLU | I | 46 | 27.814 | 22.048 | 35.389 | 1.00 | 21.24 | I | C |
| ATOM | 1939 | CB | GLU | I | 46 | 28.590 | 23.165 | 36.109 | 1.00 | 20.57 | I | C |
| ATOM | 1940 | CG | GLU | I | 46 | 30.105 | 23.101 | 36.122 | 1.00 | 25.88 | I | C |
| ATOM | 1941 | CD | GLU | I | 46 | 30.721 | 24.397 | 36.695 | 1.00 | 33.40 | I | C |
| ATOM | 1942 | OE1 | GLU | I | 46 | 30.499 | 24.672 | 37.901 | 1.00 | 25.36 | I | O |
| ATOM | 1943 | OE2 | GLU | I | 46 | 31.401 | 25.151 | 35.935 | 1.00 | 29.26 | I | O |
| ATOM | 1944 | C | GLU | I | 46 | 26.349 | 22.453 | 35.468 | 1.00 | 15.86 | I | C |
| ATOM | 1945 | O | GLU | I | 46 | 25.898 | 23.317 | 34.717 | 1.00 | 24.71 | I | O |
| ATOM | 1946 | N | TRP | I | 47 | 25.622 | 21.884 | 36.420 | 1.00 | 17.77 | I | N |
| ATOM | 1947 | CA | TRP | I | 47 | 24.219 | 22.238 | 36.626 | 1.00 | 15.27 | I | C |
| ATOM | 1948 | CB | TRP | I | 47 | 23.461 | 21.041 | 37.216 | 1.00 | 16.13 | I | C |
| ATOM | 1949 | CG | TRP | I | 47 | 21.969 | 21.259 | 37.407 | 1.00 | 21.42 | I | C |
| ATOM | 1950 | CD2 | TRP | I | 47 | 21.278 | 21.439 | 38.656 | 1.00 | 18.46 | I | C |
| ATOM | 1951 | CE2 | TRP | I | 47 | 19.907 | 21.582 | 38.358 | 1.00 | 15.96 | I | C |
| ATOM | 1952 | CE3 | TRP | I | 47 | 21.688 | 21.493 | 39.995 | 1.00 | 16.56 | I | C |
| ATOM | 1953 | CD1 | TRP | I | 47 | 21.007 | 21.303 | 36.430 | 1.00 | 14.67 | I | C |
| ATOM | 1954 | NE1 | TRP | I | 47 | 19.770 | 21.497 | 36.997 | 1.00 | 15.00 | I | N |
| ATOM | 1955 | CZ2 | TRP | I | 47 | 18.945 | 21.776 | 39.350 | 1.00 | 21.20 | I | C |
| ATOM | 1956 | CZ3 | TRP | I | 47 | 20.727 | 21.685 | 40.991 | 1.00 | 13.53 | I | C |
| ATOM | 1957 | CH2 | TRP | I | 47 | 19.376 | 21.825 | 40.662 | 1.00 | 20.91 | I | C |
| ATOM | 1958 | C | TRP | I | 47 | 24.168 | 23.428 | 37.592 | 1.00 | 16.14 | I | C |
| ATOM | 1959 | O | TRP | I | 47 | 24.832 | 23.410 | 38.625 | 1.00 | 11.35 | I | O |
| ATOM | 1960 | N | VAL | I | 48 | 23.410 | 24.470 | 37.241 | 1.00 | 18.51 | I | N |
| ATOM | 1961 | CA | VAL | I | 48 | 23.292 | 25.643 | 38.103 | 1.00 | 15.57 | I | C |
| ATOM | 1962 | CB | VAL | I | 48 | 23.201 | 26.933 | 37.276 | 1.00 | 18.17 | I | C |
| ATOM | 1963 | CG1 | VAL | I | 48 | 23.186 | 28.128 | 38.177 | 1.00 | 14.06 | I | C |
| ATOM | 1964 | CG2 | VAL | I | 48 | 24.339 | 27.019 | 36.320 | 1.00 | 7.98 | I | C |
| ATOM | 1965 | C | VAL | I | 48 | 22.037 | 25.509 | 38.965 | 1.00 | 23.88 | I | C |
| ATOM | 1966 | O | VAL | I | 48 | 22.107 | 25.582 | 40.199 | 1.00 | 17.94 | I | O |
| ATOM | 1967 | N | GLY | I | 49 | 20.900 | 25.289 | 38.300 | 1.00 | 19.71 | I | N |
| ATOM | 1968 | CA | GLY | I | 49 | 19.628 | 25.139 | 38.993 | 1.00 | 24.24 | I | C |
| ATOM | 1969 | C | GLY | I | 49 | 18.367 | 24.995 | 38.130 | 1.00 | 23.17 | I | C |
| ATOM | 1970 | O | GLY | I | 49 | 18.429 | 24.985 | 36.888 | 1.00 | 19.44 | I | O |
| ATOM | 1971 | N | LEU | I | 50 | 17.207 | 24.926 | 38.790 | 1.00 | 16.31 | I | N |
| ATOM | 1972 | CA | LEU | I | 50 | 15.943 | 24.785 | 38.086 | 1.00 | 18.56 | I | C |
| ATOM | 1973 | CB | LEU | I | 50 | 15.510 | 23.307 | 38.032 | 1.00 | 17.69 | I | C |
| ATOM | 1974 | CG | LEU | I | 50 | 15.162 | 22.547 | 39.325 | 1.00 | 15.34 | I | C |
| ATOM | 1975 | CD1 | LEU | I | 50 | 13.778 | 22.897 | 39.807 | 1.00 | 15.06 | I | C |
| ATOM | 1976 | CD2 | LEU | I | 50 | 15.239 | 21.050 | 39.091 | 1.00 | 20.45 | I | C |
| ATOM | 1977 | C | LEU | I | 50 | 14.846 | 25.581 | 38.738 | 1.00 | 20.07 | I | C |
| ATOM | 1978 | O | LEU | I | 50 | 14.989 | 26.048 | 39.863 | 1.00 | 18.29 | I | O |
| ATOM | 1979 | N | ILE | I | 51 | 13.756 | 25.748 | 37.999 | 1.00 | 22.78 | I | N |
| ATOM | 1980 | CA | ILE | I | 51 | 12.576 | 26.413 | 38.506 | 1.00 | 20.77 | I | C |
| ATOM | 1981 | CB | ILE | I | 51 | 12.340 | 27.815 | 37.910 | 1.00 | 22.86 | I | C |
| ATOM | 1982 | CG2 | ILE | I | 51 | 12.323 | 27.763 | 36.376 | 1.00 | 20.83 | I | C |
| ATOM | 1983 | CG1 | ILE | I | 51 | 11.024 | 28.371 | 38.478 | 1.00 | 23.18 | I | C |
| ATOM | 1984 | CD1 | ILE | I | 51 | 10.935 | 29.881 | 38.501 | 1.00 | 21.49 | I | C |
| ATOM | 1985 | C | ILE | I | 51 | 11.393 | 25.482 | 38.227 | 1.00 | 21.49 | I | C |
| ATOM | 1986 | O | ILE | I | 51 | 11.141 | 25.109 | 37.086 | 1.00 | 24.03 | I | O |
| ATOM | 1987 | N | ASP | I | 52 | 10.731 | 25.068 | 39.303 | 1.00 | 24.55 | I | N |
| ATOM | 1988 | CA | ASP | I | 52 | 9.574 | 24.174 | 39.294 | 1.00 | 24.00 | I | C |
| ATOM | 1989 | CB | ASP | I | 52 | 9.407 | 23.609 | 40.709 | 1.00 | 21.80 | I | C |
| ATOM | 1990 | CG | ASP | I | 52 | 8.402 | 22.465 | 40.797 | 1.00 | 18.54 | I | C |
| ATOM | 1991 | OD1 | ASP | I | 52 | 7.389 | 22.470 | 40.081 | 1.00 | 24.74 | I | O |
| ATOM | 1992 | OD2 | ASP | I | 52 | 8.612 | 21.558 | 41.626 | 1.00 | 21.82 | I | O |
| ATOM | 1993 | C | ASP | I | 52 | 8.313 | 24.943 | 38.879 | 1.00 | 27.33 | I | C |
| ATOM | 1994 | O | ASP | I | 52 | 8.024 | 26.003 | 39.413 | 1.00 | 29.94 | I | O |
| ATOM | 1995 | N | PRO | I | 52A | 7.550 | 24.411 | 37.910 | 1.00 | 30.46 | I | N |
| ATOM | 1996 | CD | PRO | I | 52A | 7.852 | 23.233 | 37.076 | 1.00 | 28.20 | I | C |
| ATOM | 1997 | CA | PRO | I | 52A | 6.327 | 25.073 | 37.446 | 1.00 | 28.67 | I | C |
| ATOM | 1998 | CB | PRO | I | 52A | 6.151 | 24.494 | 36.055 | 1.00 | 23.15 | I | C |
| ATOM | 1999 | CG | PRO | I | 52A | 6.589 | 23.084 | 36.262 | 1.00 | 27.21 | I | C |
| ATOM | 2000 | C | PRO | I | 52A | 5.092 | 24.813 | 38.314 | 1.00 | 27.87 | I | C |
| ATOM | 2001 | O | PRO | I | 52A | 4.121 | 25.555 | 38.234 | 1.00 | 29.08 | I | O |
| ATOM | 2002 | N | GLU | I | 53 | 5.136 | 23.781 | 39.150 | 1.00 | 28.36 | I | N |
| ATOM | 2003 | CA | GLU | I | 53 | 4.000 | 23.443 | 40.007 | 1.00 | 26.20 | I | C |
| ATOM | 2004 | CB | GLU | I | 53 | 4.141 | 22.056 | 40.619 | 1.00 | 28.12 | I | C |
| ATOM | 2005 | CG | GLU | I | 53 | 4.038 | 20.954 | 39.583 | 1.00 | 29.92 | I | C |
| ATOM | 2006 | CD | GLU | I | 53 | 3.916 | 19.566 | 40.179 | 1.00 | 30.51 | I | C |
| ATOM | 2007 | OE1 | GLU | I | 53 | 3.628 | 19.420 | 41.397 | 1.00 | 31.67 | I | O |
| ATOM | 2008 | OE2 | GLU | I | 53 | 4.121 | 18.615 | 39.405 | 1.00 | 28.47 | I | O |
| ATOM | 2009 | C | GLU | I | 53 | 3.715 | 24.450 | 41.095 | 1.00 | 30.41 | I | C |
| ATOM | 2010 | O | GLU | I | 53 | 2.548 | 24.691 | 41.389 | 1.00 | 29.24 | I | O |
| ATOM | 2011 | N | GLN | I | 54 | 4.758 | 25.036 | 41.690 | 1.00 | 29.61 | I | N |
| ATOM | 2012 | CA | GLN | I | 54 | 4.553 | 26.037 | 42.733 | 1.00 | 27.03 | I | C |
| ATOM | 2013 | CB | GLN | I | 54 | 4.400 | 25.398 | 44.115 | 1.00 | 24.44 | I | C |
| ATOM | 2014 | CG | GLN | I | 54 | 5.691 | 25.040 | 44.800 | 1.00 | 24.88 | I | C |
| ATOM | 2015 | CD | GLN | I | 54 | 6.524 | 24.083 | 43.991 | 1.00 | 26.71 | I | C |
| ATOM | 2016 | OE1 | GLN | I | 54 | 7.367 | 24.495 | 43.199 | 1.00 | 25.49 | I | O |
| ATOM | 2017 | NE2 | GLN | I | 54 | 6.307 | 22.801 | 44.194 | 1.00 | 21.70 | I | N |
| ATOM | 2018 | C | GLN | I | 54 | 5.586 | 27.152 | 42.781 | 1.00 | 28.33 | I | C |
| ATOM | 2019 | O | GLN | I | 54 | 5.529 | 28.006 | 43.666 | 1.00 | 33.33 | I | O |
| ATOM | 2020 | N | GLY | I | 55 | 6.531 | 27.148 | 41.847 | 1.00 | 27.79 | I | N |
| ATOM | 2021 | CA | GLY | I | 55 | 7.533 | 28.206 | 41.804 | 1.00 | 21.92 | I | C |
| ATOM | 2022 | C | GLY | I | 55 | 8.823 | 27.997 | 42.587 | 1.00 | 24.28 | I | C |
| ATOM | 2023 | O | GLY | I | 55 | 9.619 | 28.927 | 42.692 | 1.00 | 30.66 | I | O |
| ATOM | 2024 | N | ASN | I | 56 | 9.038 | 26.804 | 43.140 | 1.00 | 22.24 | I | N |
| ATOM | 2025 | CA | ASN | I | 56 | 10.259 | 26.520 | 43.898 | 1.00 | 20.38 | I | C |
| ATOM | 2026 | CB | ASN | I | 56 | 10.270 | 25.090 | 44.470 | 1.00 | 21.29 | I | C |
| ATOM | 2027 | CG | ASN | I | 56 | 9.349 | 24.918 | 45.679 | 1.00 | 22.35 | I | C |
| ATOM | 2028 | OD1 | ASN | I | 56 | 8.788 | 25.882 | 46.187 | 1.00 | 20.69 | I | O |
| ATOM | 2029 | ND2 | ASN | I | 56 | 9.214 | 23.683 | 46.151 | 1.00 | 15.28 | I | N |
| ATOM | 2030 | C | ASN | I | 56 | 11.454 | 26.669 | 42.985 | 1.00 | 26.83 | I | C |
| ATOM | 2031 | O | ASN | I | 56 | 11.358 | 26.426 | 41.786 | 1.00 | 21.43 | I | O |
| ATOM | 2032 | N | THR | I | 57 | 12.572 | 27.096 | 43.567 | 1.00 | 30.27 | I | N |
| ATOM | 2033 | CA | THR | I | 57 | 13.823 | 27.271 | 42.852 | 1.00 | 18.49 | I | C |
| ATOM | 2034 | CB | THR | I | 57 | 14.191 | 28.745 | 42.714 | 1.00 | 22.89 | I | C |
| ATOM | 2035 | OG1 | THR | I | 57 | 14.092 | 29.385 | 43.990 | 1.00 | 34.58 | I | O |
| ATOM | 2036 | CG2 | THR | I | 57 | 13.275 | 29.435 | 41.732 | 1.00 | 20.11 | I | C |
| ATOM | 2037 | C | THR | I | 57 | 14.877 | 26.525 | 43.658 | 1.00 | 24.16 | I | C |
| ATOM | 2038 | O | THR | I | 57 | 14.980 | 26.683 | 44.870 | 1.00 | 28.91 | I | O |
| ATOM | 2039 | N | ILE | I | 58 | 15.622 | 25.664 | 42.981 | 1.00 | 24.69 | I | N |
| ATOM | 2040 | CA | ILE | I | 58 | 16.645 | 24.853 | 43.606 | 1.00 | 18.01 | I | C |
| ATOM | 2041 | CB | ILE | I | 58 | 16.281 | 23.370 | 43.462 | 1.00 | 21.20 | I | C |
| ATOM | 2042 | CG2 | ILE | I | 58 | 17.244 | 22.515 | 44.222 | 1.00 | 29.30 | I | C |
| ATOM | 2043 | CG1 | ILE | I | 58 | 14.864 | 23.116 | 43.986 | 1.00 | 28.92 | I | C |
| ATOM | 2044 | CD1 | ILE | I | 58 | 14.698 | 23.326 | 45.489 | 1.00 | 29.66 | I | C |
| ATOM | 2045 | C | ILE | I | 58 | 17.967 | 25.125 | 42.888 | 1.00 | 25.28 | I | C |
| ATOM | 2046 | O | ILE | I | 58 | 18.027 | 25.170 | 41.661 | 1.00 | 28.64 | I | O |
| ATOM | 2047 | N | TYR | I | 59 | 19.039 | 25.280 | 43.653 | 1.00 | 24.51 | I | N |
| ATOM | 2048 | CA | TYR | I | 59 | 20.342 | 25.563 | 43.073 | 1.00 | 18.07 | I | C |
| ATOM | 2049 | CB | TYR | I | 59 | 20.768 | 26.986 | 43.401 | 1.00 | 17.92 | I | C |
| ATOM | 2050 | CG | TYR | I | 59 | 19.704 | 28.022 | 43.189 | 1.00 | 15.61 | I | C |
| ATOM | 2051 | CD1 | TYR | I | 59 | 18.766 | 28.289 | 44.175 | 1.00 | 8.11 | I | C |
| ATOM | 2052 | CE1 | TYR | I | 59 | 17.783 | 29.236 | 43.974 | 1.00 | 15.32 | I | C |
| ATOM | 2053 | CD2 | TYR | I | 59 | 19.629 | 28.731 | 41.989 | 1.00 | 13.79 | I | C |
| ATOM | 2054 | CE2 | TYR | I | 59 | 18.644 | 29.676 | 41.777 | 1.00 | 16.92 | I | C |
| ATOM | 2055 | CZ | TYR | I | 59 | 17.719 | 29.928 | 42.774 | 1.00 | 12.08 | I | C |
| ATOM | 2056 | OH | TYR | I | 59 | 16.737 | 30.879 | 42.565 | 1.00 | 17.49 | I | O |
| ATOM | 2057 | C | TYR | I | 59 | 21.391 | 24.657 | 43.658 | 1.00 | 19.40 | I | C |
| ATOM | 2058 | O | TYR | I | 59 | 21.155 | 23.997 | 44.657 | 1.00 | 27.42 | I | O |
| ATOM | 2059 | N | ASP | I | 60 | 22.538 | 24.581 | 43.001 | 1.00 | 23.27 | I | N |
| ATOM | 2060 | CA | ASP | I | 60 | 23.636 | 23.792 | 43.535 | 1.00 | 27.98 | I | C |
| ATOM | 2061 | CB | ASP | I | 60 | 24.736 | 23.590 | 42.477 | 1.00 | 28.16 | I | C |
| ATOM | 2062 | CG | ASP | I | 60 | 25.922 | 22.758 | 42.987 | 1.00 | 31.59 | I | C |
| ATOM | 2063 | OD1 | ASP | I | 60 | 26.148 | 22.691 | 44.215 | 1.00 | 35.48 | I | O |
| ATOM | 2064 | OD2 | ASP | I | 60 | 26.645 | 22.161 | 42.152 | 1.00 | 36.66 | I | O |
| ATOM | 2065 | C | ASP | I | 60 | 24.142 | 24.681 | 44.673 | 1.00 | 30.62 | I | C |
| ATOM | 2066 | O | ASP | I | 60 | 24.260 | 25.894 | 44.508 | 1.00 | 29.34 | I | O |
| ATOM | 2067 | N | PRO | I | 61 | 24.321 | 24.106 | 45.874 | 1.00 | 36.99 | I | N |
| ATOM | 2068 | CD | PRO | I | 61 | 23.917 | 22.738 | 46.251 | 1.00 | 38.18 | I | C |
| ATOM | 2069 | CA | PRO | I | 61 | 24.806 | 24.841 | 47.050 | 1.00 | 36.27 | I | C |
| ATOM | 2070 | CB | PRO | I | 61 | 25.015 | 23.730 | 48.072 | 1.00 | 39.47 | I | C |
| ATOM | 2071 | CG | PRO | I | 61 | 23.866 | 22.816 | 47.775 | 1.00 | 37.54 | I | C |
| ATOM | 2072 | C | PRO | I | 61 | 26.099 | 25.593 | 46.779 | 1.00 | 37.18 | I | C |
| ATOM | 2073 | O | PRO | I | 61 | 26.355 | 26.628 | 47.383 | 1.00 | 39.11 | I | O |
| ATOM | 2074 | N | LYS | I | 62 | 26.898 | 25.083 | 45.849 | 1.00 | 35.32 | I | N |
| ATOM | 2075 | CA | LYS | I | 62 | 28.160 | 25.717 | 45.484 | 1.00 | 41.89 | I | C |
| ATOM | 2076 | CB | LYS | I | 62 | 28.770 | 25.033 | 44.254 | 1.00 | 40.37 | I | C |
| ATOM | 2077 | CG | LYS | I | 62 | 29.272 | 23.632 | 44.521 | 1.00 | 52.00 | I | C |
| ATOM | 2078 | CD | LYS | I | 62 | 29.745 | 22.946 | 43.237 | 1.00 | 57.96 | I | C |
| ATOM | 2079 | CE | LYS | I | 62 | 30.138 | 21.472 | 43.478 | 1.00 | 59.72 | I | C |
| ATOM | 2080 | NZ | LYS | I | 62 | 31.333 | 21.290 | 44.367 | 1.00 | 60.21 | I | N |
| ATOM | 2081 | C | LYS | I | 62 | 28.010 | 27.210 | 45.202 | 1.00 | 39.05 | I | C |
| ATOM | 2082 | O | LYS | I | 62 | 28.913 | 27.998 | 45.483 | 1.00 | 45.91 | I | O |
| ATOM | 2083 | N | PHE | I | 63 | 26.863 | 27.593 | 44.664 | 1.00 | 36.87 | I | N |
| ATOM | 2084 | CA | PHE | I | 63 | 26.604 | 28.983 | 44.318 | 1.00 | 40.83 | I | C |
| ATOM | 2085 | CB | PHE | I | 63 | 25.548 | 29.046 | 43.206 | 1.00 | 37.06 | I | C |
| ATOM | 2086 | CG | PHE | I | 63 | 26.006 | 28.412 | 41.915 | 1.00 | 33.14 | I | C |
| ATOM | 2087 | CD1 | PHE | I | 63 | 26.815 | 29.114 | 41.033 | 1.00 | 30.97 | I | C |
| ATOM | 2088 | CD2 | PHE | I | 63 | 25.695 | 27.093 | 41.623 | 1.00 | 27.09 | I | C |
| ATOM | 2089 | CE1 | PHE | I | 63 | 27.309 | 28.513 | 39.895 | 1.00 | 28.19 | I | C |
| ATOM | 2090 | CE2 | PHE | I | 63 | 26.190 | 26.486 | 40.484 | 1.00 | 25.71 | I | C |
| ATOM | 2091 | CZ | PHE | I | 63 | 26.998 | 27.197 | 39.622 | 1.00 | 30.55 | I | C |
| ATOM | 2092 | C | PHE | I | 63 | 26.232 | 29.883 | 45.495 | 1.00 | 46.30 | I | C |
| ATOM | 2093 | O | PHE | I | 63 | 26.168 | 31.110 | 45.354 | 1.00 | 43.44 | I | O |
| ATOM | 2094 | N | GLN | I | 64 | 26.050 | 29.263 | 46.661 | 1.00 | 47.43 | I | N |
| ATOM | 2095 | CA | GLN | I | 64 | 25.676 | 29.937 | 47.902 | 1.00 | 46.34 | I | C |
| ATOM | 2096 | CB | GLN | I | 64 | 26.874 | 30.049 | 48.868 | 1.00 | 51.27 | I | C |
| ATOM | 2097 | CG | GLN | I | 64 | 28.151 | 30.687 | 48.335 | 1.00 | 57.26 | I | C |
| ATOM | 2098 | CD | GLN | I | 64 | 29.305 | 30.612 | 49.339 | 1.00 | 63.65 | I | C |
| ATOM | 2099 | OE1 | GLN | I | 64 | 30.473 | 30.766 | 48.970 | 1.00 | 63.87 | I | O |
| ATOM | 2100 | NE2 | GLN | I | 64 | 28.976 | 30.377 | 50.615 | 1.00 | 65.07 | I | N |
| ATOM | 2101 | C | GLN | I | 64 | 24.886 | 31.249 | 47.818 | 1.00 | 45.30 | I | C |
| ATOM | 2102 | O | GLN | I | 64 | 25.403 | 32.328 | 48.117 | 1.00 | 47.82 | I | O |
| ATOM | 2103 | N | ASP | I | 65 | 23.632 | 31.135 | 47.377 | 1.00 | 41.60 | I | N |
| ATOM | 2104 | CA | ASP | I | 65 | 22.712 | 32.269 | 47.281 | 1.00 | 37.88 | I | C |
| ATOM | 2105 | CB | ASP | I | 65 | 22.573 | 32.956 | 48.657 | 1.00 | 39.68 | I | C |
| ATOM | 2106 | CG | ASP | I | 65 | 22.328 | 31.966 | 49.831 | 0.50 | 37.93 | I | C |
| ATOM | 2107 | OD1 | ASP | I | 65 | 22.147 | 30.744 | 49.613 | 0.50 | 28.61 | I | O |
| ATOM | 2108 | OD2 | ASP | I | 65 | 22.325 | 32.440 | 50.997 | 0.50 | 35.67 | I | O |
| ATOM | 2109 | C | ASP | I | 65 | 23.045 | 33.334 | 46.223 | 1.00 | 34.87 | I | C |
| ATOM | 2110 | O | ASP | I | 65 | 22.312 | 34.313 | 46.063 | 1.00 | 38.81 | I | O |
| ATOM | 2111 | N | ARG | I | 66 | 24.149 | 33.164 | 45.509 | 1.00 | 31.30 | I | N |
| ATOM | 2112 | CA | ARG | I | 66 | 24.529 | 34.132 | 44.492 | 1.00 | 26.12 | I | C |
| ATOM | 2113 | CB | ARG | I | 66 | 26.004 | 33.958 | 44.106 | 1.00 | 24.90 | I | C |
| ATOM | 2114 | CG | ARG | I | 66 | 26.998 | 34.245 | 45.229 | 1.00 | 30.20 | I | C |
| ATOM | 2115 | CD | ARG | I | 66 | 28.364 | 33.576 | 44.982 | 1.00 | 29.38 | I | C |
| ATOM | 2116 | NE | ARG | I | 66 | 28.903 | 33.971 | 43.697 | 1.00 | 27.28 | I | N |
| ATOM | 2117 | CZ | ARG | I | 66 | 29.334 | 33.143 | 42.750 | 1.00 | 24.89 | I | C |
| ATOM | 2118 | NH1 | ARG | I | 66 | 29.326 | 31.820 | 42.904 | 1.00 | 18.39 | I | N |
| ATOM | 2119 | NH2 | ARG | I | 66 | 29.723 | 33.662 | 41.601 | 1.00 | 24.17 | I | N |
| ATOM | 2120 | C | ARG | I | 66 | 23.649 | 34.045 | 43.245 | 1.00 | 26.32 | I | C |
| ATOM | 2121 | O | ARG | I | 66 | 23.477 | 35.038 | 42.551 | 1.00 | 28.46 | I | O |
| ATOM | 2122 | N | ALA | I | 67 | 23.107 | 32.866 | 42.946 | 1.00 | 26.10 | I | N |
| ATOM | 2123 | CA | ALA | 1 | 67 | 22.270 | 32.724 | 41.756 | 1.00 | 25.74 | I | C |
| ATOM | 2124 | CB | ALA | I | 67 | 22.663 | 31.482 | 40.964 | 1.00 | 30.29 | I | C |
| ATOM | 2125 | C | ALA | I | 67 | 20.779 | 32.731 | 42.032 | 1.00 | 27.23 | I | C |
| ATOM | 2126 | O | ALA | I | 67 | 20.311 | 32.109 | 42.979 | 1.00 | 24.36 | I | O |
| ATOM | 2127 | N | THR | I | 68 | 20.048 | 33.471 | 41.203 | 1.00 | 25.06 | I | N |
| ATOM | 2128 | CA | THR | I | 68 | 18.605 | 33.576 | 41.316 | 1.00 | 24.25 | I | C |
| ATOM | 2129 | CB | THR | I | 68 | 18.180 | 34.977 | 41.778 | 1.00 | 22.82 | I | C |
| ATOM | 2130 | OG1 | THR | I | 68 | 18.764 | 35.232 | 43.052 | 1.00 | 28.29 | I | O |
| ATOM | 2131 | CG2 | THR | I | 68 | 16.653 | 35.087 | 41.904 | 1.00 | 20.97 | I | C |
| ATOM | 2132 | C | THR | I | 68 | 17.954 | 33.282 | 39.967 | 1.00 | 23.73 | I | C |
| ATOM | 2133 | O | THR | I | 68 | 18.187 | 33.982 | 38.993 | 1.00 | 23.15 | I | O |
| ATOM | 2134 | N | ILE | I | 69 | 17.179 | 32.208 | 39.910 | 1.00 | 22.33 | I | N |
| ATOM | 2135 | CA | ILE | I | 69 | 16.466 | 31.852 | 38.698 | 1.00 | 21.24 | I | C |
| ATOM | 2136 | CB | ILE | I | 69 | 16.515 | 30.346 | 38.465 | 1.00 | 20.81 | I | C |
| ATOM | 2137 | CG2 | ILE | I | 69 | 15.578 | 29.965 | 37.348 | 1.00 | 17.14 | I | C |
| ATOM | 2138 | CG1 | ILE | I | 69 | 17.942 | 29.921 | 38.135 | 1.00 | 15.44 | I | C |
| ATOM | 2139 | CD1 | ILE | I | 69 | 18.128 | 28.427 | 38.138 | 1.00 | 20.56 | I | C |
| ATOM | 2140 | C | ILE | I | 69 | 15.003 | 32.316 | 38.827 | 1.00 | 24.46 | I | C |
| ATOM | 2141 | O | ILE | I | 69 | 14.371 | 32.160 | 39.881 | 1.00 | 22.18 | I | O |
| ATOM | 2142 | N | SER | I | 70 | 14.484 | 32.913 | 37.762 | 1.00 | 20.87 | I | N |
| ATOM | 2143 | CA | SER | I | 70 | 13.110 | 33.406 | 37.737 | 1.00 | 19.36 | I | C |
| ATOM | 2144 | CB | SER | I | 70 | 13.068 | 34.884 | 38.174 | 1.00 | 17.16 | I | C |
| ATOM | 2145 | OG | SER | I | 70 | 13.704 | 35.754 | 37.246 | 1.00 | 22.45 | I | O |
| ATOM | 2146 | C | SER | I | 70 | 12.462 | 33.231 | 36.346 | 1.00 | 22.55 | I | C |
| ATOM | 2147 | O | SER | I | 70 | 13.083 | 32.726 | 35.407 | 1.00 | 18.94 | I | O |
| ATOM | 2148 | N | ALA | I | 71 | 11.209 | 33.648 | 36.217 | 1.00 | 25.07 | I | N |
| ATOM | 2149 | CA | ALA | I | 71 | 10.515 | 33.529 | 34.945 | 1.00 | 28.52 | I | C |
| ATOM | 2150 | CB | ALA | I | 71 | 10.081 | 32.085 | 34.717 | 1.00 | 24.08 | I | C |
| ATOM | 2151 | C | ALA | I | 71 | 9.320 | 34.462 | 34.866 | 1.00 | 29.21 | I | C |
| ATOM | 2152 | O | ALA | I | 71 | 8.661 | 34.730 | 35.861 | 1.00 | 30.74 | I | O |
| ATOM | 2153 | N | ASP | I | 72 | 9.060 | 34.958 | 33.666 | 1.00 | 31.32 | I | N |
| ATOM | 2154 | CA | ASP | I | 72 | 7.947 | 35.859 | 33.425 | 1.00 | 32.80 | I | C |
| ATOM | 2155 | CB | ASP | I | 72 | 8.473 | 37.197 | 32.919 | 1.00 | 36.03 | I | C |
| ATOM | 2156 | CG | ASP | I | 72 | 7.427 | 38.288 | 32.949 | 1.00 | 39.50 | I | C |
| ATOM | 2157 | OD1 | ASP | I | 72 | 6.513 | 38.277 | 32.097 | 1.00 | 43.05 | I | O |
| ATOM | 2158 | OD2 | ASP | I | 72 | 7.531 | 39.164 | 33.829 | 1.00 | 40.66 | I | O |
| ATOM | 2159 | C | ASP | I | 72 | 7.063 | 35.214 | 32.362 | 1.00 | 32.21 | I | C |
| ATOM | 2160 | O | ASP | I | 72 | 7.434 | 35.176 | 31.189 | 1.00 | 33.15 | I | O |
| ATOM | 2161 | N | ASN | I | 73 | 5.907 | 34.703 | 32.787 | 1.00 | 32.94 | I | N |
| ATOM | 2162 | CA | ASN | I | 73 | 4.947 | 34.045 | 31.894 | 1.00 | 30.46 | I | C |
| ATOM | 2163 | CB | ASN | I | 73 | 3.701 | 33.593 | 32.655 | 1.00 | 28.43 | I | C |
| ATOM | 2164 | CG | ASN | I | 73 | 3.981 | 32.475 | 33.632 | 1.00 | 36.44 | I | C |
| ATOM | 2165 | OD1 | ASN | I | 73 | 4.973 | 31.752 | 33.512 | 1.00 | 37.77 | I | O |
| ATOM | 2166 | ND2 | ASN | I | 73 | 3.093 | 32.314 | 34.606 | 1.00 | 41.91 | I | N |
| ATOM | 2167 | C | ASN | I | 73 | 4.500 | 34.953 | 30.756 | 1.00 | 30.91 | I | C |
| ATOM | 2168 | O | ASN | I | 73 | 4.403 | 34.519 | 29.605 | 1.00 | 32.30 | I | O |
| ATOM | 2169 | N | SER | I | 74 | 4.252 | 36.216 | 31.074 | 1.00 | 24.18 | I | N |
| ATOM | 2170 | CA | SER | I | 74 | 3.807 | 37.169 | 30.070 | 1.00 | 26.54 | I | C |
| ATOM | 2171 | CB | SER | I | 74 | 3.489 | 38.527 | 30.717 | 1.00 | 30.80 | I | C |
| ATOM | 2172 | OG | SER | I | 74 | 4.538 | 39.465 | 30.556 | 1.00 | 25.44 | I | O |
| ATOM | 2173 | C | SER | I | 74 | 4.805 | 37.339 | 28.928 | 1.00 | 32.11 | I | C |
| ATOM | 2174 | O | SER | I | 74 | 4.422 | 37.697 | 27.804 | 1.00 | 34.79 | I | O |
| ATOM | 2175 | N | LYS | I | 75 | 6.086 | 37.119 | 29.220 | 1.00 | 31.37 | I | N |
| ATOM | 2176 | CA | LYS | I | 75 | 7.122 | 37.238 | 28.200 | 1.00 | 28.87 | I | C |
| ATOM | 2177 | CB | LYS | I | 75 | 8.313 | 38.042 | 28.718 | 1.00 | 34.52 | I | C |
| ATOM | 2178 | CG | LYS | I | 75 | 7.990 | 39.473 | 29.099 | 1.00 | 35.14 | I | C |
| ATOM | 2179 | CD | LYS | I | 75 | 9.258 | 40.312 | 29.141 | 1.00 | 36.82 | I | C |
| ATOM | 2180 | CE | LYS | I | 75 | 8.967 | 41.745 | 29.516 | 1.00 | 29.69 | I | C |
| ATOM | 2181 | NZ | LYS | I | 75 | 8.670 | 41.774 | 30.947 | 1.00 | 32.48 | I | N |
| ATOM | 2182 | C | LYS | I | 75 | 7.599 | 35.865 | 27.743 | 1.00 | 29.82 | I | C |
| ATOM | 2183 | O | LYS | I | 75 | 8.409 | 35.754 | 26.810 | 1.00 | 23.93 | I | O |
| ATOM | 2184 | N | ASN | I | 76 | 7.096 | 34.822 | 28.401 | 1.00 | 25.42 | I | N |
| ATOM | 2185 | CA | ASN | I | 76 | 7.465 | 33.435 | 28.081 | 1.00 | 30.64 | I | C |
| ATOM | 2186 | CB | ASN | I | 76 | 6.874 | 33.022 | 26.711 | 1.00 | 20.90 | I | C |
| ATOM | 2187 | CG | ASN | I | 76 | 6.755 | 31.516 | 26.556 | 1.00 | 27.85 | I | C |
| ATOM | 2188 | OD1 | ASN | I | 76 | 6.514 | 30.800 | 27.526 | 1.00 | 21.69 | I | O |
| ATOM | 2189 | ND2 | ASN | I | 76 | 6.942 | 31.023 | 25.328 | 1.00 | 23.10 | I | N |
| ATOM | 2190 | C | ASN | I | 76 | 9.007 | 33.320 | 28.098 | 1.00 | 28.97 | I | C |
| ATOM | 2191 | O | ASN | I | 76 | 9.617 | 32.595 | 27.292 | 1.00 | 27.52 | I | O |
| ATOM | 2192 | N | THR | I | 77 | 9.604 | 34.022 | 29.062 | 1.00 | 28.70 | I | N |
| ATOM | 2193 | CA | THR | I | 77 | 11.047 | 34.088 | 29.236 | 1.00 | 23.07 | I | C |
| ATOM | 2194 | CB | THR | I | 77 | 11.546 | 35.535 | 28.962 | 1.00 | 21.36 | I | C |
| ATOM | 2195 | OG1 | THR | I | 77 | 11.147 | 35.926 | 27.647 | 1.00 | 24.47 | I | O |
| ATOM | 2196 | CG2 | THR | I | 77 | 13.076 | 35.660 | 29.052 | 1.00 | 14.17 | I | C |
| ATOM | 2197 | C | THR | I | 77 | 11.462 | 33.661 | 30.638 | 1.00 | 27.39 | I | C |
| ATOM | 2198 | O | THR | I | 77 | 10.736 | 33.890 | 31.617 | 1.00 | 27.43 | I | O |
| ATOM | 2199 | N | ALA | I | 78 | 12.598 | 32.970 | 30.716 | 1.00 | 22.73 | I | N |
| ATOM | 2200 | CA | ALA | I | 78 | 13.145 | 32.547 | 31.995 | 1.00 | 20.11 | I | C |
| ATOM | 2201 | CB | ALA | I | 78 | 13.482 | 31.080 | 31.964 | 1.00 | 16.78 | I | C |
| ATOM | 2202 | C | ALA | I | 78 | 14.399 | 33.398 | 32.210 | 1.00 | 22.27 | I | C |
| ATOM | 2203 | O | ALA | I | 78 | 14.982 | 33.904 | 31.248 | 1.00 | 24.29 | I | O |
| ATOM | 2204 | N | TYR | I | 79 | 14.840 | 33.527 | 33.454 | 1.00 | 20.39 | I | N |
| ATOM | 2205 | CA | TYR | I | 79 | 15.999 | 34.352 | 33.748 | 1.00 | 19.07 | I | C |
| ATOM | 2206 | CB | TYR | I | 79 | 15.532 | 35.677 | 34.386 | 1.00 | 16.35 | I | C |
| ATOM | 2207 | CG | TYR | I | 79 | 14.646 | 36.536 | 33.510 | 1.00 | 13.66 | I | C |
| ATOM | 2208 | CD1 | TYR | I | 79 | 15.191 | 37.343 | 32.533 | 1.00 | 10.22 | I | C |
| ATOM | 2209 | CE1 | TYR | I | 79 | 14.399 | 38.118 | 31.718 | 1.00 | 14.83 | I | C |
| ATOM | 2210 | CD2 | TYR | I | 79 | 13.257 | 36.527 | 33.658 | 1.00 | 20.95 | I | C |
| ATOM | 2211 | CE2 | TYR | I | 79 | 12.436 | 37.312 | 32.845 | 1.00 | 17.69 | I | C |
| ATOM | 2212 | CZ | TYR | I | 79 | 13.016 | 38.107 | 31.872 | 1.00 | 22.43 | I | C |
| ATOM | 2213 | OH | TYR | I | 79 | 12.238 | 38.908 | 31.049 | 1.00 | 22.54 | I | O |
| ATOM | 2214 | C | TYR | I | 79 | 16.981 | 33.693 | 34.700 | 1.00 | 18.71 | I | C |
| ATOM | 2215 | O | TYR | I | 79 | 16.600 | 32.863 | 35.507 | 1.00 | 24.34 | I | O |
| ATOM | 2216 | N | LEU | I | 80 | 18.251 | 34.065 | 34.600 | 1.00 | 17.95 | I | N |
| ATOM | 2217 | CA | LEU | I | 80 | 19.273 | 33.570 | 35.521 | 1.00 | 16.71 | I | C |
| ATOM | 2218 | CB | LEU | I | 80 | 20.220 | 32.528 | 34.906 | 1.00 | 14.98 | I | C |
| ATOM | 2219 | CG | LEU | I | 80 | 21.469 | 32.280 | 35.794 | 1.00 | 21.13 | I | C |
| ATOM | 2220 | CD1 | LEU | I | 80 | 21.085 | 31.675 | 37.167 | 1.00 | 14.99 | I | C |
| ATOM | 2221 | CD2 | LEU | I | 80 | 22.501 | 31.392 | 35.111 | 1.00 | 5.22 | I | C |
| ATOM | 2222 | C | LEU | I | 80 | 20.056 | 34.810 | 35.921 | 1.00 | 20.77 | I | C |
| ATOM | 2223 | O | LEU | I | 80 | 20.544 | 35.557 | 35.066 | 1.00 | 21.30 | I | O |
| ATOM | 2224 | N | GLN | I | 81 | 20.122 | 35.043 | 37.228 | 1.00 | 21.05 | I | N |
| ATOM | 2225 | CA | GLN | I | 81 | 20.811 | 36.188 | 37.796 | 1.00 | 24.56 | I | C |
| ATOM | 2226 | CB | GLN | I | 81 | 19.806 | 36.985 | 38.618 | 1.00 | 25.14 | I | C |
| ATOM | 2227 | CG | GLN | I | 81 | 19.984 | 38.493 | 38.606 | 1.00 | 40.49 | I | C |
| ATOM | 2228 | CD | GLN | I | 81 | 20.624 | 39.022 | 39.880 | 1.00 | 46.05 | I | C |
| ATOM | 2229 | OE1 | GLN | I | 81 | 21.381 | 39.997 | 39.852 | 1.00 | 47.88 | I | O |
| ATOM | 2230 | NE2 | GLN | I | 81 | 20.319 | 38.384 | 41.007 | 1.00 | 46.07 | I | N |
| ATOM | 2231 | C | GLN | I | 81 | 21.950 | 35.681 | 38.688 | 1.00 | 28.04 | I | C |
| ATOM | 2232 | O | GLN | I | 81 | 21.767 | 34.727 | 39.448 | 1.00 | 29.81 | I | O |
| ATOM | 2233 | N | MET | I | 82 | 23.137 | 36.270 | 38.561 | 1.00 | 27.17 | I | N |
| ATOM | 2234 | CA | MET | I | 82 | 24.250 | 35.861 | 39.399 | 1.00 | 28.66 | I | C |
| ATOM | 2235 | CB | MET | I | 82 | 25.131 | 34.830 | 38.684 | 1.00 | 33.51 | I | C |
| ATOM | 2236 | CG | MET | I | 82 | 25.552 | 33.673 | 39.605 | 1.00 | 45.13 | I | C |
| ATOM | 2237 | SD | MET | I | 82 | 26.775 | 32.483 | 38.958 | 1.00 | 49.56 | I | S |
| ATOM | 2238 | CE | MET | I | 82 | 25.698 | 31.497 | 37.914 | 1.00 | 50.90 | I | C |
| ATOM | 2239 | C | MET | I | 82 | 25.047 | 37.090 | 39.849 | 1.00 | 29.64 | I | C |
| ATOM | 2240 | O | MET | I | 82 | 25.350 | 37.965 | 39.045 | 1.00 | 31.26 | I | O |
| ATOM | 2241 | N | ASN | I | 82A | 25.322 | 37.181 | 41.149 | 1.00 | 29.35 | I | N |
| ATOM | 2242 | CA | ASN | I | 82A | 26.072 | 38.305 | 41.723 | 1.00 | 31.51 | I | C |
| ATOM | 2243 | CB | ASN | I | 82A | 25.361 | 38.853 | 42.965 | 1.00 | 36.56 | I | C |
| ATOM | 2244 | CG | ASN | I | 82A | 23.950 | 39.343 | 42.673 | 1.00 | 45.77 | I | C |
| ATOM | 2245 | OD1 | ASN | I | 82A | 23.078 | 39.283 | 43.540 | 1.00 | 49.74 | I | O |
| ATOM | 2246 | ND2 | ASN | I | 82A | 23.721 | 39.838 | 41.458 | 1.00 | 47.58 | I | N |
| ATOM | 2247 | C | ASN | I | 82A | 27.477 | 37.879 | 42.136 | 1.00 | 29.53 | I | C |
| ATOM | 2248 | O | ASN | I | 82A | 27.791 | 36.683 | 42.119 | 1.00 | 23.22 | I | O |
| ATOM | 2249 | N | SER | I | 828 | 28.301 | 38.861 | 42.527 | 1.00 | 21.13 | I | N |
| ATOM | 2250 | CA | SER | I | 82B | 29.683 | 38.623 | 42.975 | 1.00 | 22.94 | I | C |
| ATOM | 2251 | CB | SER | I | 82B | 29.687 | 38.128 | 44.422 | 1.00 | 14.20 | I | C |
| ATOM | 2252 | OG | SER | I | 82B | 28.957 | 39.009 | 45.246 | 1.00 | 23.89 | I | O |
| ATOM | 2253 | C | SER | I | 82B | 30.439 | 37.625 | 42.087 | 1.00 | 26.20 | I | C |
| ATOM | 2254 | O | SER | I | 828 | 31.025 | 36.643 | 42.575 | 1.00 | 23.64 | I | O |
| ATOM | 2255 | N | LEU | I | 82C | 30.430 | 37.890 | 40.785 | 1.00 | 20.50 | I | N |
| ATOM | 2256 | CA | LEU | I | 82C | 31.077 | 37.012 | 39.833 | 1.00 | 21.89 | I | C |
| ATOM | 2257 | CB | LEU | I | 82C | 30.734 | 37.434 | 38.392 | 1.00 | 22.68 | I | C |
| ATOM | 2258 | CG | LEU | I | 82C | 29.241 | 37.325 | 38.019 | 1.00 | 19.51 | I | C |
| ATOM | 2259 | CD1 | LEU | I | 82C | 28.981 | 37.954 | 36.689 | 1.00 | 23.72 | I | C |
| ATOM | 2260 | CD2 | LEU | I | 82C | 28.782 | 35.889 | 38.024 | 1.00 | 14.28 | I | C |
| ATOM | 2261 | C | LEU | I | 82C | 32.583 | 36.930 | 40.003 | 1.00 | 28.52 | I | C |
| ATOM | 2262 | O | LEU | I | 82C | 33.247 | 37.908 | 40.370 | 1.00 | 21.12 | I | O |
| ATOM | 2263 | N | ARG | I | 83 | 33.094 | 35.715 | 39.847 | 1.00 | 28.19 | I | N |
| ATOM | 2264 | CA | ARG | I | 83 | 34.519 | 35.487 | 39.894 | 1.00 | 31.45 | I | C |
| ATOM | 2265 | CB | ARG | I | 83 | 34.971 | 34.736 | 41.144 | 1.00 | 34.94 | I | C |
| ATOM | 2266 | CG | ARG | I | 83 | 34.107 | 33.634 | 41.637 | 1.00 | 40.16 | I | C |
| ATOM | 2267 | CD | ARG | I | 83 | 34.732 | 33.102 | 42.907 | 1.00 | 43.21 | I | C |
| ATOM | 2268 | NE | ARG | I | 83 | 34.137 | 31.849 | 43.353 | 1.00 | 45.85 | I | N |
| ATOM | 2269 | CZ | ARG | I | 83 | 33.113 | 31.761 | 44.199 | 1.00 | 47.06 | I | C |
| ATOM | 2270 | NH1 | ARG | I | 83 | 32.548 | 32.858 | 44.703 | 1.00 | 42.59 | I | N |
| ATOM | 2271 | NH2 | ARG | I | 83 | 32.676 | 30.565 | 44.568 | 1.00 | 47.91 | I | N |
| ATOM | 2272 | C | ARG | I | 83 | 34.969 | 34.800 | 38.622 | 1.00 | 32.94 | I | C |
| ATOM | 2273 | O | ARG | I | 83 | 34.143 | 34.426 | 37.777 | 1.00 | 36.43 | I | O |
| ATOM | 2274 | N | ALA | I | 84 | 36.284 | 34.719 | 38.457 | 1.00 | 30.89 | I | N |
| ATOM | 2275 | CA | ALA | I | 84 | 36.925 | 34.111 | 37.293 | 1.00 | 27.53 | I | C |
| ATOM | 2276 | CB | ALA | I | 84 | 38.423 | 33.961 | 37.575 | 1.00 | 14.89 | I | C |
| ATOM | 2277 | C | ALA | I | 84 | 36.325 | 32.764 | 36.866 | 1.00 | 24.91 | I | C |
| ATOM | 2278 | O | ALA | I | 84 | 36.157 | 32.512 | 35.681 | 1.00 | 27.90 | I | O |
| ATOM | 2279 | N | GLU | I | 85 | 36.006 | 31.917 | 37.841 | 1.00 | 26.50 | I | N |
| ATOM | 2280 | CA | GLU | I | 85 | 35.435 | 30.595 | 37.593 | 1.00 | 29.91 | I | C |
| ATOM | 2281 | CB | GLU | I | 85 | 35.493 | 29.736 | 38.867 | 1.00 | 28.16 | I | C |
| ATOM | 2282 | CG | GLU | I | 85 | 34.784 | 30.362 | 40.067 | 1.00 | 33.94 | I | C |
| ATOM | 2283 | CD | GLU | I | 85 | 33.989 | 29.361 | 40.880 | 1.00 | 31.78 | I | C |
| ATOM | 2284 | OE1 | GLU | I | 85 | 34.591 | 28.649 | 41.710 | 1.00 | 32.37 | I | O |
| ATOM | 2285 | OE2 | GLU | I | 85 | 32.755 | 29.295 | 40.695 | 1.00 | 32.24 | I | O |
| ATOM | 2286 | C | GLU | I | 85 | 34.000 | 30.623 | 37.065 | 1.00 | 25.59 | I | C |
| ATOM | 2287 | O | GLU | I | 85 | 33.434 | 29.579 | 36.761 | 1.00 | 31.79 | I | O |
| ATOM | 2288 | N | ASP | I | 86 | 33.397 | 31.800 | 37.004 | 1.00 | 22.38 | I | N |
| ATOM | 2289 | CA | ASP | I | 86 | 32.035 | 31.899 | 36.506 | 1.00 | 20.85 | I | C |
| ATOM | 2290 | CB | ASP | I | 86 | 31.227 | 32.981 | 37.220 | 1.00 | 21.21 | I | C |
| ATOM | 2291 | CG | ASP | I | 86 | 31.086 | 32.731 | 38.712 | 1.00 | 23.53 | I | C |
| ATOM | 2292 | OD1 | ASP | I | 86 | 30.794 | 31.591 | 39.139 | 1.00 | 27.30 | I | O |
| ATOM | 2293 | OD2 | ASP | I | 86 | 31.259 | 33.700 | 39.472 | 1.00 | 26.37 | I | O |
| ATOM | 2294 | C | ASP | I | 86 | 32.101 | 32.188 | 35.020 | 1.00 | 26.06 | I | C |
| ATOM | 2295 | O | ASP | I | 86 | 31.071 | 32.252 | 34.354 | 1.00 | 22.80 | I | O |
| ATOM | 2296 | N | THR | I | 87 | 33.305 | 32.438 | 34.514 | 1.00 | 12.51 | I | N |
| ATOM | 2297 | CA | THR | I | 87 | 33.474 | 32.654 | 33.081 | 1.00 | 22.76 | I | C |
| ATOM | 2298 | CB | THR | I | 87 | 34.948 | 32.971 | 32.717 | 1.00 | 17.47 | I | C |
| ATOM | 2299 | OG1 | THR | I | 87 | 35.256 | 34.319 | 33.115 | 1.00 | 25.63 | I | O |
| ATOM | 2300 | CG2 | THR | I | 87 | 35.180 | 32.812 | 31.222 | 1.00 | 18.45 | I | C |
| ATOM | 2301 | C | THR | I | 87 | 33.027 | 31.356 | 32.392 | 1.00 | 18.40 | I | C |
| ATOM | 2302 | O | THR | I | 87 | 33.625 | 30.300 | 32.607 | 1.00 | 20.86 | I | O |
| ATOM | 2303 | N | ALA | I | 88 | 31.967 | 31.440 | 31.590 | 1.00 | 15.58 | I | N |
| ATOM | 2304 | CA | ALA | I | 88 | 31.430 | 30.251 | 30.932 | 1.00 | 13.44 | I | C |
| ATOM | 2305 | CB | ALA | I | 88 | 30.916 | 29.289 | 31.969 | 1.00 | 9.23 | I | C |
| ATOM | 2306 | C | ALA | I | 88 | 30.318 | 30.511 | 29.951 | 1.00 | 13.53 | I | C |
| ATOM | 2307 | O | ALA | I | 88 | 29.752 | 31.607 | 29.893 | 1.00 | 13.35 | I | O |
| ATOM | 2308 | N | VAL | I | 89 | 30.043 | 29.491 | 29.145 | 1.00 | 9.76 | I | N |
| ATOM | 2309 | CA | VAL | I | 89 | 28.947 | 29.563 | 28.189 | 1.00 | 13.85 | I | C |
| ATOM | 2310 | CB | VAL | I | 89 | 29.158 | 28.607 | 27.002 | 1.00 | 19.13 | I | C |
| ATOM | 2311 | CG1 | VAL | I | 89 | 27.873 | 28.532 | 26.162 | 1.00 | 18.41 | I | C |
| ATOM | 2312 | CG2 | VAL | I | 89 | 30.360 | 29.083 | 26.150 | 1.00 | 8.08 | I | C |
| ATOM | 2313 | C | VAL | I | 89 | 27.782 | 29.087 | 29.026 | 1.00 | 11.85 | I | C |
| ATOM | 2314 | O | VAL | I | 89 | 27.864 | 28.026 | 29.658 | 1.00 | 18.84 | I | O |
| ATOM | 2315 | N | TYR | I | 90 | 26.760 | 29.918 | 29.151 | 1.00 | 12.30 | I | N |
| ATOM | 2316 | CA | TYR | I | 90 | 25.589 | 29.541 | 29.945 | 1.00 | 12.36 | I | C |
| ATOM | 2317 | CB | TYR | I | 90 | 25.128 | 30.702 | 30.840 | 1.00 | 12.25 | I | C |
| ATOM | 2318 | CG | TYR | I | 90 | 26.026 | 30.959 | 32.035 | 1.00 | 12.53 | I | C |
| ATOM | 2319 | CD1 | TYR | I | 90 | 27.262 | 31.561 | 31.883 | 1.00 | 9.23 | I | C |
| ATOM | 2320 | CE1 | TYR | I | 90 | 28.103 | 31.724 | 32.954 | 1.00 | 10.22 | I | C |
| ATOM | 2321 | CD2 | TYR | I | 90 | 25.654 | 30.542 | 33.305 | 1.00 | 12.86 | I | C |
| ATOM | 2322 | CE2 | TYR | I | 90 | 26.482 | 30.697 | 34.383 | 1.00 | 14.66 | I | C |
| ATOM | 2323 | CZ | TYR | I | 90 | 27.716 | 31.282 | 34.202 | 1.00 | 18.48 | I | C |
| ATOM | 2324 | OH | TYR | I | 90 | 28.602 | 31.334 | 35.253 | 1.00 | 21.37 | I | O |
| ATOM | 2325 | C | TYR | I | 90 | 24.449 | 29.107 | 29.038 | 1.00 | 14.24 | I | C |
| ATOM | 2326 | O | TYR | I | 90 | 24.083 | 29.808 | 28.093 | 1.00 | 12.84 | I | O |
| ATOM | 2327 | N | TYR | I | 91 | 23.891 | 27.947 | 29.335 | 1.00 | 16.49 | I | N |
| ATOM | 2328 | CA | TYR | I | 91 | 22.775 | 27.425 | 28.578 | 1.00 | 18.31 | I | C |
| ATOM | 2329 | CB | TYR | I | 91 | 23.023 | 25.985 | 28.162 | 1.00 | 14.07 | I | C |
| ATOM | 2330 | CG | TYR | I | 91 | 24.159 | 25.816 | 27.217 | 1.00 | 18.79 | I | C |
| ATOM | 2331 | CD1 | TYR | I | 91 | 24.002 | 26.062 | 25.861 | 1.00 | 15.57 | I | C |
| ATOM | 2332 | CE1 | TYR | I | 91 | 25.052 | 25.834 | 24.974 | 1.00 | 15.45 | I | C |
| ATOM | 2333 | CD2 | TYR | I | 91 | 25.389 | 25.346 | 27.670 | 1.00 | 17.39 | I | C |
| ATOM | 2334 | CE2 | TYR | I | 91 | 26.423 | 25.116 | 26.808 | 1.00 | 17.31 | I | C |
| ATOM | 2335 | CZ | TYR | I | 91 | 26.255 | 25.360 | 25.461 | 1.00 | 18.08 | I | C |
| ATOM | 2336 | OH | TYR | I | 91 | 27.302 | 25.110 | 24.617 | 1.00 | 16.45 | I | O |
| ATOM | 2337 | C | TYR | I | 91 | 21.516 | 27.429 | 29.402 | 1.00 | 17.80 | I | C |
| ATOM | 2338 | O | TYR | I | 91 | 21.534 | 27.226 | 30.609 | 1.00 | 22.64 | I | O |
| ATOM | 2339 | N | CYS | I | 92 | 20.423 | 27.505 | 28.674 | 1.00 | 21.32 | I | N |
| ATOM | 2340 | CA | CYS | I | 92 | 19.070 | 27.487 | 29.190 | 1.00 | 24.31 | I | C |
| ATOM | 2341 | C | CYS | I | 92 | 18.530 | 26.166 | 28.653 | 1.00 | 13.88 | I | C |
| ATOM | 2342 | O | CYS | I | 92 | 18.662 | 25.897 | 27.462 | 1.00 | 20.99 | I | O |
| ATOM | 2343 | CB | CYS | I | 92 | 18.326 | 28.651 | 28.540 | 1.00 | 25.62 | I | C |
| ATOM | 2344 | SG | CYS | I | 92 | 16.562 | 28.698 | 28.851 | 1.00 | 50.43 | I | S |
| ATOM | 2345 | N | ALA | I | 93 | 17.953 | 25.333 | 29.507 | 1.00 | 12.89 | I | N |
| ATOM | 2346 | CA | ALA | I | 93 | 17.420 | 24.052 | 29.046 | 1.00 | 11.49 | I | C |
| ATOM | 2347 | CB | ALA | I | 93 | 18.343 | 22.949 | 29.417 | 1.00 | 13.44 | I | C |
| ATOM | 2348 | C | ALA | I | 93 | 16.029 | 23.711 | 29.543 | 1.00 | 18.34 | I | C |
| ATOM | 2349 | O | ALA | I | 93 | 15.693 | 24.002 | 30.690 | 1.00 | 17.18 | I | O |
| ATOM | 2350 | N | ARG | I | 94 | 15.229 | 23.088 | 28.673 | 1.00 | 19.24 | I | N |
| ATOM | 2351 | CA | ARG | I | 94 | 13.881 | 22.658 | 29.034 | 1.00 | 11.38 | I | C |
| ATOM | 2352 | CB | ARG | I | 94 | 12.906 | 22.784 | 27.867 | 1.00 | 13.47 | I | C |
| ATOM | 2353 | CG | ARG | I | 94 | 11.436 | 22.707 | 28.299 | 1.00 | 16.00 | I | C |
| ATOM | 2354 | CD | ARG | I | 94 | 10.490 | 22.755 | 27.117 | 1.00 | 19.50 | I | C |
| ATOM | 2355 | NE | ARG | I | 94 | 10.423 | 21.464 | 26.440 | 1.00 | 23.77 | I | N |
| ATOM | 2356 | CZ | ARG | I | 94 | 9.516 | 21.144 | 25.520 | 1.00 | 20.37 | I | C |
| ATOM | 2357 | NH1 | ARG | I | 94 | 8.600 | 22.031 | 25.162 | 1.00 | 19.96 | I | N |
| ATOM | 2358 | NH2 | ARG | I | 94 | 9.515 | 19.928 | 24.975 | 1.00 | 11.59 | I | N |
| ATOM | 2359 | C | ARG | I | 94 | 13.967 | 21.208 | 29.474 | 1.00 | 13.83 | I | C |
| ATOM | 2360 | O | ARG | I | 94 | 14.698 | 20.399 | 28.890 | 1.00 | 17.06 | I | O |
| ATOM | 2361 | N | ASP | I | 95 | 13.208 | 20.886 | 30.511 | 1.00 | 15.87 | I | N |
| ATOM | 2362 | CA | ASP | I | 95 | 13.182 | 19.551 | 31.084 | 1.00 | 16.13 | I | C |
| ATOM | 2363 | CB | ASP | I | 95 | 13.522 | 19.711 | 32.588 | 1.00 | 16.76 | I | C |
| ATOM | 2364 | CG | ASP | I | 95 | 13.074 | 18.545 | 33.502 | 1.00 | 23.86 | I | C |
| ATOM | 2365 | OD1 | ASP | I | 95 | 12.389 | 17.590 | 33.098 | 1.00 | 19.13 | I | O |
| ATOM | 2366 | OD2 | ASP | I | 95 | 13.404 | 18.628 | 34.712 | 1.00 | 20.66 | I | O |
| ATOM | 2367 | C | ASP | I | 95 | 11.853 | 18.832 | 30.829 | 1.00 | 15.93 | I | C |
| ATOM | 2368 | O | ASP | I | 95 | 10.782 | 19.409 | 30.931 | 1.00 | 19.54 | I | O |
| ATOM | 2369 | N | THR | I | 96 | 11.964 | 17.578 | 30.419 | 1.00 | 18.09 | I | N |
| ATOM | 2370 | CA | THR | I | 96 | 10.828 | 16.694 | 30.233 | 1.00 | 16.21 | I | C |
| ATOM | 2371 | CB | THR | I | 96 | 10.468 | 16.452 | 28.746 | 1.00 | 22.22 | I | C |
| ATOM | 2372 | OG1 | THR | I | 96 | 9.710 | 17.562 | 28.262 | 1.00 | 20.69 | I | O |
| ATOM | 2373 | CG2 | THR | I | 96 | 9.617 | 15.174 | 28.583 | 1.00 | 20.65 | I | C |
| ATOM | 2374 | C | THR | I | 96 | 11.353 | 15.411 | 30.856 | 1.00 | 16.49 | I | C |
| ATOM | 2375 | O | THR | I | 96 | 12.422 | 14.951 | 30.481 | 1.00 | 21.97 | I | O |
| ATOM | 2376 | N | ALA | I | 97 | 10.659 | 14.907 | 31.874 | 1.00 | 11.30 | I | N |
| ATOM | 2377 | CA | ALA | I | 97 | 11.042 | 13.675 | 32.558 | 1.00 | 13.80 | I | C |
| ATOM | 2378 | CB | ALA | I | 97 | 10.793 | 12.472 | 31.666 | 1.00 | 18.72 | I | C |
| ATOM | 2379 | C | ALA | I | 97 | 12.477 | 13.683 | 33.079 | 1.00 | 15.44 | I | C |
| ATOM | 2380 | O | ALA | I | 97 | 13.150 | 12.656 | 33.084 | 1.00 | 20.81 | I | O |
| ATOM | 2381 | N | ALA | I | 98 | 12.918 | 14.858 | 33.521 | 1.00 | 15.73 | I | N |
| ATOM | 2382 | CA | ALA | I | 98 | 14.240 | 15.070 | 34.098 | 1.00 | 19.79 | I | C |
| ATOM | 2383 | CB | ALA | I | 98 | 14.446 | 14.174 | 35.329 | 1.00 | 22.77 | I | C |
| ATOM | 2384 | C | ALA | I | 98 | 15.439 | 15.007 | 33.160 | 1.00 | 23.01 | I | C |
| ATOM | 2385 | O | ALA | I | 98 | 16.583 | 14.948 | 33.617 | 1.00 | 21.50 | I | O |
| ATOM | 2386 | N | TYR | I | 99 | 15.182 | 14.979 | 31.854 | 1.00 | 19.07 | I | N |
| ATOM | 2387 | CA | TYR | I | 99 | 16.268 | 15.010 | 30.882 | 1.00 | 16.94 | I | C |
| ATOM | 2388 | CB | TYR | I | 99 | 16.288 | 13.759 | 29.999 | 1.00 | 13.00 | I | C |
| ATOM | 2389 | CG | TYR | I | 99 | 15.136 | 13.622 | 29.027 | 1.00 | 20.31 | I | C |
| ATOM | 2390 | CD1 | TYR | I | 99 | 15.132 | 14.310 | 27.811 | 1.00 | 17.01 | I | C |
| ATOM | 2391 | CE1 | TYR | I | 99 | 14.117 | 14.120 | 26.880 | 1.00 | 18.50 | I | C |
| ATOM | 2392 | CD2 | TYR | I | 99 | 14.082 | 12.743 | 29.288 | 1.00 | 18.37 | I | C |
| ATOM | 2393 | CE2 | TYR | I | 99 | 13.065 | 12.548 | 28.364 | 1.00 | 22.55 | I | C |
| ATOM | 2394 | CZ | TYR | I | 99 | 13.084 | 13.234 | 27.165 | 1.00 | 18.22 | I | C |
| ATOM | 2395 | OH | TYR | I | 99 | 12.052 | 13.038 | 26.275 | 1.00 | 22.05 | I | O |
| ATOM | 2396 | C | TYR | I | 99 | 16.045 | 16.301 | 30.087 | 1.00 | 20.93 | I | C |
| ATOM | 2397 | O | TYR | I | 99 | 14.921 | 16.820 | 30.056 | 1.00 | 18.90 | I | O |
| ATOM | 2398 | N | PHE | I | 100 | 17.102 | 16.850 | 29.493 | 1.00 | 15.59 | I | N |
| ATOM | 2399 | CA | PHE | I | 100 | 16.967 | 18.098 | 28.750 | 1.00 | 18.02 | I | C |
| ATOM | 2400 | CB | PHE | I | 100 | 18.278 | 18.905 | 28.832 | 1.00 | 14.29 | I | C |
| ATOM | 2401 | CG | PHE | I | 100 | 18.743 | 19.187 | 30.246 | 1.00 | 22.11 | I | C |
| ATOM | 2402 | CD1 | PHE | I | 100 | 17.843 | 19.611 | 31.228 | 1.00 | 21.53 | I | C |
| ATOM | 2403 | CD2 | PHE | I | 100 | 20.071 | 18.970 | 30.610 | 1.00 | 15.67 | I | C |
| ATOM | 2404 | CE1 | PHE | I | 100 | 18.262 | 19.801 | 32.540 | 1.00 | 18.41 | I | C |
| ATOM | 2405 | CE2 | PHE | I | 100 | 20.494 | 19.155 | 31.907 | 1.00 | 16.07 | I | C |
| ATOM | 2406 | CZ | PHE | I | 100 | 19.596 | 19.569 | 32.880 | 1.00 | 17.51 | I | C |
| ATOM | 2407 | C | PHE | I | 100 | 16.582 | 17.875 | 27.286 | 1.00 | 17.36 | I | C |
| ATOM | 2408 | O | PHE | I | 100 | 17.408 | 17.407 | 26.515 | 1.00 | 24.48 | I | O |
| ATOM | 2409 | N | ASP | I | 101 | 15.356 | 18.237 | 26.891 | 1.00 | 19.81 | I | N |
| ATOM | 2410 | CA | ASP | I | 101 | 14.934 | 18.056 | 25.496 | 1.00 | 16.13 | I | C |
| ATOM | 2411 | CB | ASP | I | 101 | 13.468 | 17.596 | 25.373 | 1.00 | 17.30 | I | C |
| ATOM | 2412 | CG | ASP | I | 101 | 12.456 | 18.502 | 26.097 | 1.00 | 26.46 | I | C |
| ATOM | 2413 | OD1 | ASP | I | 101 | 12.782 | 19.612 | 26.580 | 1.00 | 25.12 | I | O |
| ATOM | 2414 | OD2 | ASP | I | 101 | 11.278 | 18.079 | 26.165 | 1.00 | 27.46 | I | O |
| ATOM | 2415 | C | ASP | I | 101 | 15.257 | 19.212 | 24.541 | 1.00 | 20.35 | I | C |
| ATOM | 2416 | O | ASP | I | 101 | 15.420 | 19.009 | 23.341 | 1.00 | 27.21 | I | O |
| ATOM | 2417 | N | TYR | I | 102 | 15.327 | 20.437 | 25.052 | 1.00 | 23.21 | I | N |
| ATOM | 2418 | CA | TYR | I | 102 | 15.692 | 21.562 | 24.195 | 1.00 | 20.19 | I | C |
| ATOM | 2419 | CB | TYR | I | 102 | 14.483 | 22.344 | 23.696 | 1.00 | 19.74 | I | C |
| ATOM | 2420 | CG | TYR | I | 102 | 13.649 | 21.541 | 22.747 | 1.00 | 26.72 | I | C |
| ATOM | 2421 | CD1 | TYR | I | 102 | 13.873 | 21.584 | 21.360 | 1.00 | 26.67 | I | C |
| ATOM | 2422 | CE1 | TYR | I | 102 | 13.109 | 20.807 | 20.492 | 1.00 | 18.46 | I | C |
| ATOM | 2423 | CD2 | TYR | I | 102 | 12.648 | 20.708 | 23.231 | 1.00 | 23.76 | I | C |
| ATOM | 2424 | CE2 | TYR | I | 102 | 11.893 | 19.940 | 22.387 | 1.00 | 26.25 | I | C |
| ATOM | 2425 | CZ | TYR | I | 102 | 12.119 | 19.984 | 21.023 | 1.00 | 25.47 | I | C |
| ATOM | 2426 | OH | TYR | I | 102 | 11.344 | 19.176 | 20.225 | 1.00 | 25.43 | I | O |
| ATOM | 2427 | C | TYR | I | 102 | 16.678 | 22.480 | 24.868 | 1.00 | 18.69 | I | C |
| ATOM | 2428 | O | TYR | I | 102 | 16.699 | 22.606 | 26.086 | 1.00 | 17.61 | I | O |
| ATOM | 2429 | N | TRP | I | 103 | 17.489 | 23.142 | 24.058 | 1.00 | 19.41 | I | N |
| ATOM | 2430 | CA | TRP | I | 103 | 18.516 | 24.019 | 24.589 | 1.00 | 14.05 | I | C |
| ATOM | 2431 | CB | TRP | I | 103 | 19.898 | 23.365 | 24.458 | 1.00 | 14.08 | I | C |
| ATOM | 2432 | CG | TRP | I | 103 | 20.085 | 22.061 | 25.121 | 1.00 | 14.80 | I | C |
| ATOM | 2433 | CD2 | TRP | I | 103 | 20.885 | 21.797 | 26.290 | 1.00 | 18.05 | I | C |
| ATOM | 2434 | CE2 | TRP | I | 103 | 20.810 | 20.412 | 26.544 | 1.00 | 17.29 | I | C |
| ATOM | 2435 | CE3 | TRP | I | 103 | 21.660 | 22.597 | 27.137 | 1.00 | 17.89 | I | C |
| ATOM | 2436 | CD1 | TRP | I | 103 | 19.574 | 20.865 | 24.724 | 1.00 | 8.22 | I | C |
| ATOM | 2437 | NE1 | TRP | I | 103 | 20.009 | 19.863 | 25.574 | 1.00 | 16.71 | I | N |
| ATOM | 2438 | CZ2 | TRP | I | 103 | 21.473 | 19.806 | 27.614 | 1.00 | 17.47 | I | C |
| ATOM | 2439 | CZ3 | TRP | I | 103 | 22.325 | 21.995 | 28.201 | 1.00 | 20.43 | I | C |
| ATOM | 2440 | CH2 | TRP | I | 103 | 22.223 | 20.611 | 28.428 | 1.00 | 23.04 | I | C |
| ATOM | 2441 | C | TRP | I | 103 | 18.608 | 25.282 | 23.808 | 1.00 | 9.36 | I | C |
| ATOM | 2442 | O | TRP | I | 103 | 18.358 | 25.290 | 22.616 | 1.00 | 12.51 | I | O |
| ATOM | 2443 | N | GLY | I | 104 | 19.024 | 26.353 | 24.477 | 1.00 | 17.52 | I | N |
| ATOM | 2444 | CA | GLY | I | 104 | 19.237 | 27.605 | 23.764 | 1.00 | 17.11 | I | C |
| ATOM | 2445 | C | GLY | I | 104 | 20.595 | 27.506 | 23.061 | 1.00 | 19.76 | I | C |
| ATOM | 2446 | O | GLY | I | 104 | 21.330 | 26.522 | 23.224 | 1.00 | 14.37 | I | O |
| ATOM | 2447 | N | GLN | I | 105 | 20.951 | 28.512 | 22.281 | 1.00 | 16.99 | I | N |
| ATOM | 2448 | CA | GLN | I | 105 | 22.228 | 28.473 | 21.590 | 1.00 | 23.41 | I | C |
| ATOM | 2449 | CB | GLN | I | 105 | 22.272 | 29.554 | 20.518 | 1.00 | 23.68 | I | C |
| ATOM | 2450 | CG | GLN | I | 105 | 22.561 | 30.965 | 21.040 | 1.00 | 22.28 | I | C |
| ATOM | 2451 | CD | GLN | I | 105 | 21.368 | 31.614 | 21.707 | 1.00 | 25.55 | I | C |
| ATOM | 2452 | OE1 | GLN | I | 105 | 20.428 | 30.939 | 22.139 | 1.00 | 24.30 | I | O |
| ATOM | 2453 | NE2 | GLN | I | 105 | 21.389 | 32.941 | 21.773 | 1.00 | 21.40 | I | N |
| ATOM | 2454 | C | GLN | I | 105 | 23.421 | 28.633 | 22.548 | 1.00 | 24.40 | I | C |
| ATOM | 2455 | O | GLN | I | 105 | 24.505 | 28.143 | 22.276 | 1.00 | 29.59 | I | O |
| ATOM | 2456 | N | GLY | I | 106 | 23.206 | 29.308 | 23.672 | 1.00 | 27.29 | I | N |
| ATOM | 2457 | CA | GLY | I | 106 | 24.275 | 29.525 | 24.637 | 1.00 | 28.13 | I | C |
| ATOM | 2458 | C | GLY | I | 106 | 24.824 | 30.944 | 24.624 | 1.00 | 27.44 | I | C |
| ATOM | 2459 | O | GLY | I | 106 | 25.001 | 31.558 | 23.571 | 1.00 | 24.35 | I | O |
| ATOM | 2460 | N | THR | I | 107 | 25.018 | 31.505 | 25.804 | 1.00 | 23.54 | I | N |
| ATOM | 2461 | CA | THR | I | 107 | 25.556 | 32.852 | 25.890 | 1.00 | 23.27 | I | C |
| ATOM | 2462 | CB | THR | I | 107 | 24.491 | 33.843 | 26.374 | 1.00 | 21.72 | I | C |
| ATOM | 2463 | OG1 | THR | I | 107 | 25.010 | 35.173 | 26.301 | 1.00 | 28.72 | I | O |
| ATOM | 2464 | CG2 | THR | I | 107 | 24.045 | 33.533 | 27.782 | 1.00 | 24.92 | I | C |
| ATOM | 2465 | C | THR | I | 107 | 26.834 | 32.879 | 26.744 | 1.00 | 23.59 | I | C |
| ATOM | 2466 | O | THR | I | 107 | 26.889 | 32.315 | 27.845 | 1.00 | 22.20 | I | O |
| ATOM | 2467 | N | LEU | I | 108 | 27.897 | 33.436 | 26.171 | 1.00 | 23.06 | I | N |
| ATOM | 2468 | CA | LEU | I | 108 | 29.186 | 33.518 | 26.851 | 1.00 | 20.40 | I | C |
| ATOM | 2469 | CB | LEU | I | 108 | 30.322 | 33.649 | 25.827 | 1.00 | 18.21 | I | C |
| ATOM | 2470 | CG | LEU | I | 108 | 31.760 | 33.829 | 26.342 | 1.00 | 18.84 | I | C |
| ATOM | 2471 | CD1 | LEU | I | 108 | 32.214 | 32.612 | 27.154 | 1.00 | 11.23 | I | C |
| ATOM | 2472 | CD2 | LEU | I | 108 | 32.692 | 34.031 | 25.179 | 1.00 | 13.20 | I | C |
| ATOM | 2473 | C | LEU | I | 108 | 29.270 | 34.700 | 27.808 | 1.00 | 25.17 | I | C |
| ATOM | 2474 | O | LEU | I | 108 | 28.991 | 35.829 | 27.410 | 1.00 | 25.06 | I | O |
| ATOM | 2475 | N | VAL | I | 109 | 29.608 | 34.418 | 29.069 | 1.00 | 17.79 | I | N |
| ATOM | 2476 | CA | VAL | I | 109 | 29.802 | 35.437 | 30.086 | 1.00 | 14.26 | I | C |
| ATOM | 2477 | CB | VAL | I | 109 | 28.948 | 35.174 | 31.346 | 1.00 | 16.13 | I | C |
| ATOM | 2478 | CG1 | VAL | I | 109 | 29.362 | 36.096 | 32.459 | 1.00 | 12.88 | I | C |
| ATOM | 2479 | CG2 | VAL | I | 109 | 27.498 | 35.429 | 31.055 | 1.00 | 19.64 | I | C |
| ATOM | 2480 | C | VAL | I | 109 | 31.300 | 35.383 | 30.450 | 1.00 | 18.82 | I | C |
| ATOM | 2481 | O | VAL | I | 109 | 31.809 | 34.361 | 30.917 | 1.00 | 14.90 | I | O |
| ATOM | 2482 | N | THR | I | 110 | 32.010 | 36.469 | 30.187 | 1.00 | 20.53 | I | N |
| ATOM | 2483 | CA | THR | I | 110 | 33.450 | 36.547 | 30.467 | 1.00 | 24.37 | I | C |
| ATOM | 2484 | CB | THR | I | 110 | 34.244 | 37.156 | 29.240 | 1.00 | 20.28 | I | C |
| ATOM | 2485 | OG1 | THR | I | 110 | 34.013 | 36.361 | 28.079 | 1.00 | 25.76 | I | O |
| ATOM | 2486 | CG2 | THR | I | 110 | 35.728 | 37.163 | 29.479 | 1.00 | 16.25 | I | C |
| ATOM | 2487 | C | THR | I | 110 | 33.648 | 37.447 | 31.675 | 1.00 | 21.59 | I | C |
| ATOM | 2488 | O | THR | I | 110 | 33.203 | 38.593 | 31.662 | 1.00 | 22.19 | I | O |
| ATOM | 2489 | N | VAL | I | 111 | 34.254 | 36.916 | 32.734 | 1.00 | 23.62 | I | N |
| ATOM | 2490 | CA | VAL | I | 111 | 34.515 | 37.719 | 33.934 | 1.00 | 24.04 | I | C |
| ATOM | 2491 | CB | VAL | I | 111 | 34.075 | 37.017 | 35.202 | 1.00 | 27.63 | I | C |
| ATOM | 2492 | CG1 | VAL | I | 111 | 34.375 | 37.911 | 36.412 | 1.00 | 25.80 | I | C |
| ATOM | 2493 | CG2 | VAL | I | 111 | 32.567 | 36.703 | 35.118 | 1.00 | 23.78 | I | C |
| ATOM | 2494 | C | VAL | I | 111 | 35.994 | 38.068 | 34.019 | 1.00 | 22.34 | I | C |
| ATOM | 2495 | O | VAL | I | 111 | 36.835 | 37.200 | 34.252 | 1.00 | 21.95 | I | O |
| ATOM | 2496 | N | SER | I | 112 | 36.299 | 39.351 | 33.859 | 1.00 | 22.45 | I | N |
| ATOM | 2497 | CA | SER | I | 112 | 37.683 | 39.808 | 33.847 | 1.00 | 22.51 | I | C |
| ATOM | 2498 | CB | SER | I | 112 | 38.303 | 39.395 | 32.509 | 1.00 | 20.51 | I | C |
| ATOM | 2499 | OG | SER | I | 112 | 39.553 | 39.997 | 32.272 | 1.00 | 23.44 | I | O |
| ATOM | 2500 | C | SER | I | 112 | 37.809 | 41.315 | 34.020 | 1.00 | 19.99 | I | C |
| ATOM | 2501 | O | SER | I | 112 | 36.923 | 42.062 | 33.639 | 1.00 | 21.88 | I | O |
| ATOM | 2502 | N | SER | I | 113 | 38.920 | 41.761 | 34.590 | 1.00 | 19.19 | I | N |
| ATOM | 2503 | CA | SER | I | 113 | 39.145 | 43.196 | 34.764 | 1.00 | 22.88 | I | C |
| ATOM | 2504 | CB | SER | I | 113 | 39.703 | 43.516 | 36.164 | 1.00 | 26.37 | I | C |
| ATOM | 2505 | OG | SER | I | 113 | 40.846 | 42.740 | 36.499 | 1.00 | 28.45 | I | O |
| ATOM | 2506 | C | SER | I | 113 | 40.048 | 43.763 | 33.652 | 1.00 | 24.75 | I | C |
| ATOM | 2507 | O | SER | I | 113 | 40.362 | 44.956 | 33.631 | 1.00 | 25.34 | I | O |
| ATOM | 2508 | N | ALA | I | 114 | 40.414 | 42.897 | 32.711 | 1.00 | 22.22 | I | N |
| ATOM | 2509 | CA | ALA | I | 114 | 41.247 | 43.244 | 31.558 | 1.00 | 22.62 | I | C |
| ATOM | 2510 | CB | ALA | I | 114 | 41.575 | 41.989 | 30.780 | 1.00 | 23.61 | I | C |
| ATOM | 2511 | C | ALA | I | 114 | 40.597 | 44.249 | 30.622 | 1.00 | 22.60 | I | C |
| ATOM | 2512 | O | ALA | I | 114 | 39.376 | 44.257 | 30.452 | 1.00 | 32.84 | I | O |
| ATOM | 2513 | N | SER | I | 115 | 41.431 | 45.077 | 29.996 | 1.00 | 35.26 | I | N |
| ATOM | 2514 | CA | SER | I | 115 | 40.983 | 46.107 | 29.045 | 1.00 | 34.92 | I | C |
| ATOM | 2515 | CB | SER | I | 115 | 41.703 | 47.439 | 29.310 | 1.00 | 33.54 | I | C |
| ATOM | 2516 | OG | SER | I | 115 | 41.382 | 47.920 | 30.611 | 1.00 | 40.83 | I | O |
| ATOM | 2517 | C | SER | I | 115 | 41.244 | 45.649 | 27.613 | 1.00 | 33.22 | I | C |
| ATOM | 2518 | O | SER | I | 115 | 42.121 | 44.810 | 27.380 | 1.00 | 29.64 | I | O |
| ATOM | 2519 | N | THR | I | 116 | 40.483 | 46.189 | 26.660 | 1.00 | 32.09 | I | N |
| ATOM | 2520 | CA | THR | I | 116 | 40.655 | 45.805 | 25.260 | 1.00 | 31.84 | I | C |
| ATOM | 2521 | CB | THR | I | 116 | 39.583 | 46.430 | 24.338 | 1.00 | 37.44 | I | C |
| ATOM | 2522 | OG1 | THR | I | 116 | 38.297 | 45.920 | 24.707 | 1.00 | 42.97 | I | O |
| ATOM | 2523 | CG2 | THR | I | 116 | 39.837 | 46.063 | 22.864 | 1.00 | 37.00 | I | C |
| ATOM | 2524 | C | THR | I | 116 | 42.059 | 46.158 | 24.790 | 1.00 | 32.93 | I | C |
| ATOM | 2525 | O | THR | I | 116 | 42.553 | 47.265 | 25.037 | 1.00 | 26.61 | I | O |
| ATOM | 2526 | N | LYS | I | 117 | 42.695 | 45.191 | 24.131 | 1.00 | 28.99 | I | N |
| ATOM | 2527 | CA | LYS | I | 117 | 44.055 | 45.348 | 23.659 | 1.00 | 27.91 | I | C |
| ATOM | 2528 | CB | LYS | I | 117 | 45.016 | 44.979 | 24.792 | 1.00 | 32.29 | I | C |
| ATOM | 2529 | CG | LYS | I | 117 | 46.481 | 45.316 | 24.535 | 1.00 | 35.88 | I | C |
| ATOM | 2530 | CD | LYS | I | 117 | 47.336 | 44.989 | 25.766 | 1.00 | 41.08 | I | C |
| ATOM | 2531 | CE | LYS | I | 117 | 48.822 | 44.902 | 25.406 | 1.00 | 42.78 | I | C |
| ATOM | 2532 | NZ | LYS | I | 117 | 49.093 | 43.795 | 24.428 | 1.00 | 35.19 | I | N |
| ATOM | 2533 | C | LYS | I | 117 | 44.365 | 44.504 | 22.429 | 1.00 | 28.21 | I | C |
| ATOM | 2534 | O | LYS | I | 117 | 44.149 | 43.289 | 22.414 | 1.00 | 26.00 | I | O |
| ATOM | 2535 | N | GLY | I | 118 | 44.864 | 45.172 | 21.394 | 1.00 | 26.51 | I | N |
| ATOM | 2536 | CA | GLY | I | 118 | 45.237 | 44.492 | 20.172 | 1.00 | 28.11 | I | C |
| ATOM | 2537 | C | GLY | I | 118 | 46.498 | 43.682 | 20.395 | 1.00 | 31.65 | I | C |
| ATOM | 2538 | O | GLY | I | 118 | 47.319 | 44.030 | 21.249 | 1.00 | 34.44 | I | O |
| ATOM | 2539 | N | PRO | I | 119 | 46.697 | 42.606 | 19.621 | 1.00 | 31.37 | I | N |
| ATOM | 2540 | CD | PRO | I | 119 | 45.840 | 42.147 | 18.511 | 1.00 | 29.07 | I | C |
| ATOM | 2541 | CA | PRO | I | 119 | 47.876 | 41.747 | 19.753 | 1.00 | 33.19 | I | C |
| ATOM | 2542 | CB | PRO | I | 119 | 47.439 | 40.488 | 19.010 | 1.00 | 31.10 | I | C |
| ATOM | 2543 | CG | PRO | I | 119 | 46.714 | 41.086 | 17.834 | 1.00 | 30.43 | I | C |
| ATOM | 2544 | C | PRO | I | 119 | 49.144 | 42.277 | 19.094 | 1.00 | 33.88 | I | C |
| ATOM | 2545 | O | PRO | I | 119 | 49.101 | 43.108 | 18.185 | 1.00 | 33.90 | I | O |
| ATOM | 2546 | N | SER | I | 120 | 50.272 | 41.790 | 19.593 | 1.00 | 34.49 | I | N |
| ATOM | 2547 | CA | SER | I | 120 | 51.578 | 42.076 | 19.022 | 1.00 | 30.59 | I | C |
| ATOM | 2548 | CB | SER | I | 120 | 52.653 | 42.141 | 20.115 | 1.00 | 31.06 | I | C |
| ATOM | 2549 | OG | SER | I | 120 | 52.354 | 43.115 | 21.102 | 1.00 | 41.94 | I | O |
| ATOM | 2550 | C | SER | I | 120 | 51.744 | 40.782 | 18.203 | 1.00 | 28.70 | I | C |
| ATOM | 2551 | O | SER | I | 120 | 51.380 | 39.693 | 18.676 | 1.00 | 25.03 | I | O |
| ATOM | 2552 | N | VAL | I | 121 | 52.182 | 40.894 | 16.955 | 1.00 | 26.74 | I | N |
| ATOM | 2553 | CA | VAL | I | 121 | 52.356 | 39.705 | 16.132 | 1.00 | 23.77 | I | C |
| ATOM | 2554 | CB | VAL | I | 121 | 51.510 | 39.786 | 14.851 | 1.00 | 22.76 | I | C |
| ATOM | 2555 | CG1 | VAL | I | 121 | 51.617 | 38.499 | 14.081 | 1.00 | 15.79 | I | C |
| ATOM | 2556 | CG2 | VAL | I | 121 | 50.050 | 40.063 | 15.217 | 1.00 | 20.92 | I | C |
| ATOM | 2557 | C | VAL | I | 121 | 53.829 | 39.521 | 15.805 | 1.00 | 24.88 | I | C |
| ATOM | 2558 | O | VAL | I | 121 | 54.423 | 40.358 | 15.135 | 1.00 | 29.02 | I | O |
| ATOM | 2559 | N | PHE | I | 122 | 54.404 | 38.429 | 16.308 | 1.00 | 22.62 | I | N |
| ATOM | 2560 | CA | PHE | I | 122 | 55.824 | 38.104 | 16.125 | 1.00 | 26.77 | I | C |
| ATOM | 2561 | CB | PHE | I | 122 | 56.500 | 37.827 | 17.487 | 1.00 | 22.02 | I | C |
| ATOM | 2562 | CG | PHE | I | 122 | 56.305 | 38.922 | 18.508 | 1.00 | 20.44 | I | C |
| ATOM | 2563 | CD1 | PHE | I | 122 | 56.812 | 40.189 | 18.299 | 1.00 | 21.91 | I | C |
| ATOM | 2564 | CD2 | PHE | I | 122 | 55.621 | 38.675 | 19.689 | 1.00 | 22.22 | I | C |
| ATOM | 2565 | CE1 | PHE | I | 122 | 56.641 | 41.197 | 19.256 | 1.00 | 17.37 | I | C |
| ATOM | 2566 | CE2 | PHE | I | 122 | 55.451 | 39.675 | 20.645 | 1.00 | 16.14 | I | C |
| ATOM | 2567 | CZ | PHE | I | 122 | 55.963 | 40.933 | 20.423 | 1.00 | 20.02 | I | C |
| ATOM | 2568 | C | PHE | I | 122 | 56.052 | 36.890 | 15.222 | 1.00 | 29.38 | I | C |
| ATOM | 2569 | O | PHE | I | 122 | 55.296 | 35.915 | 15.254 | 1.00 | 33.57 | I | O |
| ATOM | 2570 | N | PRO | I | 123 | 57.152 | 36.905 | 14.457 | 1.00 | 33.83 | I | N |
| ATOM | 2571 | CD | PRO | I | 123 | 58.156 | 37.980 | 14.403 | 1.00 | 33.34 | I | C |
| ATOM | 2572 | CA | PRO | I | 123 | 57.503 | 35.812 | 13.544 | 1.00 | 32.62 | I | C |
| ATOM | 2573 | CB | PRO | I | 123 | 58.633 | 36.402 | 12.683 | 1.00 | 33.92 | I | C |
| ATOM | 2574 | CG | PRO | I | 123 | 58.653 | 37.881 | 12.997 | 1.00 | 37.35 | I | C |
| ATOM | 2575 | C | PRO | I | 123 | 58.067 | 34.604 | 14.259 | 1.00 | 34.12 | I | C |
| ATOM | 2576 | O | PRO | I | 123 | 58.761 | 34.724 | 15.269 | 1.00 | 31.06 | I | O |
| ATOM | 2577 | N | LEU | I | 124 | 57.773 | 33.442 | 13.692 | 1.00 | 34.14 | I | N |
| ATOM | 2578 | CA | LEU | I | 124 | 58.284 | 32.163 | 14.145 | 1.00 | 32.32 | I | C |
| ATOM | 2579 | CB | LEU | I | 124 | 57.141 | 31.189 | 14.467 | 1.00 | 28.30 | I | C |
| ATOM | 2580 | CG | LEU | I | 124 | 56.221 | 31.439 | 15.673 | 1.00 | 30.04 | I | C |
| ATOM | 2581 | CD1 | LEU | I | 124 | 55.088 | 30.429 | 15.697 | 1.00 | 21.41 | I | C |
| ATOM | 2582 | CD2 | LEU | I | 124 | 56.996 | 31.349 | 16.961 | 1.00 | 24.91 | I | C |
| ATOM | 2583 | C | LEU | I | 124 | 58.989 | 31.770 | 12.841 | 1.00 | 34.32 | I | C |
| ATOM | 2584 | O | LEU | I | 124 | 58.414 | 31.093 | 11.995 | 1.00 | 40.73 | I | O |
| ATOM | 2585 | N | ALA | I | 125 | 60.183 | 32.322 | 12.630 | 1.00 | 37.49 | I | N |
| ATOM | 2586 | CA | ALA | I | 125 | 60.972 | 32.091 | 11.411 | 1.00 | 35.26 | I | C |
| ATOM | 2587 | CB | ALA | I | 125 | 62.072 | 33.118 | 11.322 | 1.00 | 37.18 | I | C |
| ATOM | 2588 | C | ALA | I | 125 | 61.566 | 30.699 | 11.247 | 1.00 | 38.57 | I | C |
| ATOM | 2589 | O | ALA | I | 125 | 61.997 | 30.084 | 12.228 | 1.00 | 44.21 | I | O |
| ATOM | 2590 | N | PRO | I | 126 | 61.635 | 30.202 | 9.991 | 1.00 | 39.06 | I | N |
| ATOM | 2591 | CD | PRO | I | 126 | 61.146 | 30.888 | 8.780 | 1.00 | 39.83 | I | C |
| ATOM | 2592 | CA | PRO | I | 126 | 62.180 | 28.878 | 9.651 | 1.00 | 41.96 | I | C |
| ATOM | 2593 | CB | PRO | I | 126 | 62.148 | 28.881 | 8.128 | 1.00 | 42.54 | I | C |
| ATOM | 2594 | CG | PRO | I | 126 | 60.985 | 29.750 | 7.814 | 1.00 | 43.27 | I | C |
| ATOM | 2595 | C | PRO | I | 126 | 63.606 | 28.696 | 10.160 | 1.00 | 44.67 | I | C |
| ATOM | 2596 | O | PRO | I | 126 | 64.372 | 29.658 | 10.227 | 1.00 | 49.96 | I | O |
| ATOM | 2597 | N | SER | I | 127 | 63.966 | 27.464 | 10.502 | 1.00 | 47.52 | I | N |
| ATOM | 2598 | CA | SER | I | 127 | 65.300 | 27.168 | 11.020 | 1.00 | 56.83 | I | C |
| ATOM | 2599 | CB | SER | I | 127 | 65.322 | 25.766 | 11.635 | 1.00 | 56.04 | I | C |
| ATOM | 2600 | OG | SER | I | 127 | 66.393 | 25.630 | 12.555 | 1.00 | 57.42 | I | O |
| ATOM | 2601 | C | SER | I | 127 | 66.432 | 27.322 | 9.990 | 1.00 | 60.92 | I | C |
| ATOM | 2602 | O | SER | I | 127 | 67.576 | 27.636 | 10.349 | 1.00 | 65.50 | I | O |
| ATOM | 2603 | N | SER | I | 128 | 66.105 | 27.113 | 8.715 | 1.00 | 66.67 | I | N |
| ATOM | 2604 | CA | SER | I | 128 | 67.074 | 27.229 | 7.622 | 1.00 | 70.89 | I | C |
| ATOM | 2605 | CB | SER | I | 128 | 67.537 | 28.682 | 7.461 | 1.00 | 71.11 | I | C |
| ATOM | 2606 | OG | SER | I | 128 | 66.495 | 29.487 | 6.935 | 1.00 | 73.26 | I | O |
| ATOM | 2607 | C | SER | I | 128 | 68.283 | 26.311 | 7.789 | 1.00 | 70.90 | I | C |
| ATOM | 2608 | O | SER | I | 128 | 68.760 | 25.727 | 6.815 | 1.00 | 72.65 | I | O |
| ATOM | 2609 | N | GLY | I | 133 | 65.417 | 17.065 | 6.067 | 1.00 | 73.22 | I | N |
| ATOM | 2610 | CA | GLY | I | 133 | 64.121 | 16.853 | 5.441 | 1.00 | 71.73 | I | C |
| ATOM | 2611 | C | GLY | I | 133 | 63.740 | 17.972 | 4.485 | 1.00 | 72.83 | I | C |
| ATOM | 2612 | O | GLY | I | 133 | 64.332 | 19.060 | 4.514 | 1.00 | 74.58 | I | O |
| ATOM | 2613 | N | GLY | I | 134 | 62.749 | 17.710 | 3.635 | 1.00 | 69.79 | I | N |
| ATOM | 2614 | CA | GLY | I | 134 | 62.305 | 18.711 | 2.679 | 1.00 | 65.06 | I | C |
| ATOM | 2615 | C | GLY | I | 134 | 61.161 | 19.584 | 3.167 | 1.00 | 62.05 | I | C |
| ATOM | 2616 | O | GLY | I | 134 | 60.566 | 20.320 | 2.377 | 1.00 | 61.32 | I | O |
| ATOM | 2617 | N | THR | I | 135 | 60.859 | 19.518 | 4.465 | 1.00 | 59.31 | I | N |
| ATOM | 2618 | CA | THR | I | 135 | 59.768 | 20.307 | 5.048 | 1.00 | 55.55 | I | C |
| ATOM | 2619 | CB | THR | I | 135 | 58.615 | 19.406 | 5.545 | 1.00 | 53.36 | I | C |
| ATOM | 2620 | OG1 | THR | I | 135 | 58.331 | 18.408 | 4.563 | 1.00 | 56.88 | I | O |
| ATOM | 2621 | CG2 | THR | I | 135 | 57.359 | 20.228 | 5.762 | 1.00 | 53.95 | I | C |
| ATOM | 2622 | C | THR | I | 135 | 60.195 | 21.206 | 6.213 | 1.00 | 50.92 | I | C |
| ATOM | 2623 | O | THR | I | 135 | 60.878 | 20.774 | 7.153 | 1.00 | 47.44 | I | O |
| ATOM | 2624 | N | ALA | I | 136 | 59.770 | 22.459 | 6.141 | 1.00 | 46.44 | I | N |
| ATOM | 2625 | CA | ALA | I | 136 | 60.069 | 23.429 | 7.183 | 1.00 | 46.19 | I | C |
| ATOM | 2626 | CB | ALA | I | 136 | 60.919 | 24.564 | 6.618 | 1.00 | 49.96 | I | C |
| ATOM | 2627 | C | ALA | I | 136 | 58.762 | 23.981 | 7.770 | 1.00 | 43.87 | I | C |
| ATOM | 2628 | O | ALA | I | 136 | 57.700 | 23.930 | 7.138 | 1.00 | 25.26 | I | O |
| ATOM | 2629 | N | ALA | I | 137 | 58.840 | 24.456 | 9.009 | 1.00 | 40.72 | I | N |
| ATOM | 2630 | CA | ALA | I | 137 | 57.685 | 25.039 | 9.674 | 1.00 | 37.99 | I | C |
| ATOM | 2631 | CB | ALA | I | 137 | 57.369 | 24.287 | 10.956 | 1.00 | 31.35 | I | C |
| ATOM | 2632 | C | ALA | I | 137 | 58.006 | 26.490 | 9.975 | 1.00 | 36.47 | I | C |
| ATOM | 2633 | O | ALA | I | 137 | 59.146 | 26.831 | 10.289 | 1.00 | 40.33 | I | O |
| ATOM | 2634 | N | LEU | I | 138 | 57.011 | 27.351 | 9.821 | 1.00 | 35.05 | I | N |
| ATOM | 2635 | CA | LEU | I | 138 | 57.171 | 28.772 | 10.111 | 1.00 | 30.38 | I | C |
| ATOM | 2636 | CB | LEU | I | 138 | 57.539 | 29.548 | 8.849 | 1.00 | 32.90 | I | C |
| ATOM | 2637 | CG | LEU | I | 138 | 56.567 | 29.524 | 7.674 | 1.00 | 36.03 | I | C |
| ATOM | 2638 | CD1 | LEU | I | 138 | 55.644 | 30.725 | 7.729 | 1.00 | 37.42 | I | C |
| ATOM | 2639 | CD2 | LEU | I | 138 | 57.366 | 29.542 | 6.399 | 1.00 | 33.98 | I | C |
| ATOM | 2640 | C | LEU | I | 138 | 55.845 | 29.243 | 10.681 | 1.00 | 31.77 | I | C |
| ATOM | 2641 | O | LEU | I | 138 | 54.850 | 28.513 | 10.617 | 1.00 | 30.12 | I | O |
| ATOM | 2642 | N | GLY | I | 139 | 55.822 | 30.440 | 11.257 | 1.00 | 27.72 | I | N |
| ATOM | 2643 | CA | GLY | I | 139 | 54.585 | 30.914 | 11.840 | 1.00 | 28.53 | I | C |
| ATOM | 2644 | C | GLY | I | 139 | 54.531 | 32.292 | 12.466 | 1.00 | 30.52 | I | C |
| ATOM | 2645 | O | GLY | I | 139 | 55.404 | 33.137 | 12.260 | 1.00 | 28.07 | I | O |
| ATOM | 2646 | N | CYS | I | 140 | 53.463 | 32.510 | 13.227 | 1.00 | 30.32 | I | N |
| ATOM | 2647 | CA | CYS | I | 140 | 53.212 | 33.772 | 13.904 | 1.00 | 32.76 | I | C |
| ATOM | 2648 | C | CYS | I | 140 | 52.718 | 33.560 | 15.319 | 1.00 | 32.05 | I | C |
| ATOM | 2649 | O | CYS | I | 140 | 51.859 | 32.707 | 15.581 | 1.00 | 29.21 | I | O |
| ATOM | 2650 | CB | CYS | I | 140 | 52.159 | 34.585 | 13.158 | 1.00 | 34.49 | I | C |
| ATOM | 2651 | SG | CYS | I | 140 | 52.774 | 35.376 | 11.654 | 1.00 | 44.32 | I | S |
| ATOM | 2652 | N | LEU | I | 141 | 53.276 | 34.346 | 16.228 | 1.00 | 23.49 | I | N |
| ATOM | 2653 | CA | LEU | I | 141 | 52.887 | 34.292 | 17.622 | 1.00 | 22.64 | I | C |
| ATOM | 2654 | CB | LEU | I | 141 | 54.134 | 34.291 | 18.516 | 1.00 | 16.63 | I | C |
| ATOM | 2655 | CG | LEU | I | 141 | 53.925 | 34.489 | 20.021 | 1.00 | 18.04 | I | C |
| ATOM | 2656 | CD1 | LEU | I | 141 | 53.331 | 33.256 | 20.677 | 1.00 | 13.00 | I | C |
| ATOM | 2657 | CD2 | LEU | I | 141 | 55.271 | 34.846 | 20.671 | 1.00 | 20.28 | I | C |
| ATOM | 2658 | C | LEU | I | 141 | 52.040 | 35.551 | 17.812 | 1.00 | 23.00 | I | C |
| ATOM | 2659 | O | LEU | I | 141 | 52.533 | 36.674 | 17.684 | 1.00 | 23.80 | I | O |
| ATOM | 2660 | N | VAL | I | 142 | 50.739 | 35.349 | 17.984 | 1.00 | 25.54 | I | N |
| ATOM | 2661 | CA | VAL | I | 142 | 49.787 | 36.444 | 18.165 | 1.00 | 19.79 | I | C |
| ATOM | 2662 | CB | VAL | I | 142 | 48.505 | 36.094 | 17.430 | 1.00 | 15.33 | I | C |
| ATOM | 2663 | CG1 | VAL | I | 142 | 47.528 | 37.242 | 17.431 | 1.00 | 16.01 | I | C |
| ATOM | 2664 | CG2 | VAL | I | 142 | 48.867 | 35.751 | 15.992 | 1.00 | 16.05 | I | C |
| ATOM | 2665 | C | VAL | I | 142 | 49.638 | 36.567 | 19.674 | 1.00 | 24.17 | I | C |
| ATOM | 2666 | O | VAL | I | 142 | 48.916 | 35.807 | 20.307 | 1.00 | 28.38 | I | O |
| ATOM | 2667 | N | LYS | I | 143 | 50.366 | 37.525 | 20.237 | 1.00 | 22.55 | I | N |
| ATOM | 2668 | CA | LYS | I | 143 | 50.437 | 37.701 | 21.678 | 1.00 | 24.22 | I | C |
| ATOM | 2669 | CB | LYS | I | 143 | 51.920 | 37.601 | 22.080 | 1.00 | 25.34 | I | C |
| ATOM | 2670 | CG | LYS | I | 143 | 52.217 | 37.690 | 23.561 | 1.00 | 26.27 | I | C |
| ATOM | 2671 | CD | LYS | I | 143 | 53.576 | 37.091 | 23.870 | 1.00 | 33.06 | I | C |
| ATOM | 2672 | CE | LYS | I | 143 | 54.012 | 37.365 | 25.307 | 1.00 | 39.73 | I | C |
| ATOM | 2673 | NZ | LYS | I | 143 | 53.141 | 36.692 | 26.314 | 1.00 | 33.06 | I | N |
| ATOM | 2674 | C | LYS | I | 143 | 49.819 | 38.917 | 22.355 | 1.00 | 27.69 | I | C |
| ATOM | 2675 | O | LYS | I | 143 | 49.790 | 40.023 | 21.806 | 1.00 | 31.82 | I | O |
| ATOM | 2676 | N | ASP | I | 144 | 49.365 | 38.685 | 23.587 | 1.00 | 29.18 | I | N |
| ATOM | 2677 | CA | ASP | I | 144 | 48.797 | 39.701 | 24.470 | 1.00 | 24.84 | I | C |
| ATOM | 2678 | CB | ASP | I | 144 | 49.902 | 40.690 | 24.908 | 1.00 | 27.79 | I | C |
| ATOM | 2679 | CG | ASP | I | 144 | 50.944 | 40.059 | 25.850 | 1.00 | 31.03 | I | C |
| ATOM | 2680 | OD1 | ASP | I | 144 | 50.677 | 38.987 | 26.441 | 1.00 | 27.17 | I | O |
| ATOM | 2681 | OD2 | ASP | I | 144 | 52.036 | 40.661 | 26.018 | 1.00 | 36.87 | I | O |
| ATOM | 2682 | C | ASP | I | 144 | 47.586 | 40.469 | 23.965 | 1.00 | 20.81 | I | C |
| ATOM | 2683 | O | ASP | I | 144 | 47.621 | 41.691 | 23.833 | 1.00 | 20.01 | I | O |
| ATOM | 2684 | N | TYR | I | 145 | 46.500 | 39.762 | 23.695 | 1.00 | 23.75 | I | N |
| ATOM | 2685 | CA | TYR | I | 145 | 45.302 | 40.442 | 23.228 | 1.00 | 20.69 | I | C |
| ATOM | 2686 | CB | TYR | I | 145 | 44.991 | 40.095 | 21.763 | 1.00 | 20.35 | I | C |
| ATOM | 2687 | CG | TYR | I | 145 | 44.667 | 38.642 | 21.510 | 1.00 | 21.37 | I | C |
| ATOM | 2688 | CD1 | TYR | I | 145 | 45.675 | 37.735 | 21.191 | 1.00 | 20.86 | I | C |
| ATOM | 2689 | CE1 | TYR | I | 145 | 45.383 | 36.385 | 20.953 | 1.00 | 23.41 | I | C |
| ATOM | 2690 | CD2 | TYR | I | 145 | 43.343 | 38.166 | 21.585 | 1.00 | 22.21 | I | C |
| ATOM | 2691 | CE2 | TYR | I | 145 | 43.043 | 36.819 | 21.344 | 1.00 | 18.42 | I | C |
| ATOM | 2692 | CZ | TYR | I | 145 | 44.074 | 35.940 | 21.030 | 1.00 | 23.03 | I | C |
| ATOM | 2693 | OH | TYR | I | 145 | 43.818 | 34.612 | 20.786 | 1.00 | 27.41 | I | O |
| ATOM | 2694 | C | TYR | I | 145 | 44.120 | 40.129 | 24.122 | 1.00 | 22.27 | I | C |
| ATOM | 2695 | O | TYR | I | 145 | 44.139 | 39.156 | 24.880 | 1.00 | 19.29 | I | O |
| ATOM | 2696 | N | PHE | I | 146 | 43.122 | 41.003 | 24.058 | 1.00 | 24.69 | I | N |
| ATOM | 2697 | CA | PHE | I | 146 | 41.895 | 40.864 | 24.822 | 1.00 | 21.68 | I | C |
| ATOM | 2698 | CB | PHE | I | 146 | 42.076 | 41.252 | 26.275 | 1.00 | 15.87 | I | C |
| ATOM | 2699 | CG | PHE | I | 146 | 40.861 | 41.000 | 27.104 | 1.00 | 16.35 | I | C |
| ATOM | 2700 | CD1 | PHE | I | 146 | 39.878 | 41.971 | 27.231 | 1.00 | 19.01 | I | C |
| ATOM | 2701 | CD2 | PHE | I | 146 | 40.687 | 39.774 | 27.737 | 1.00 | 16.87 | I | C |
| ATOM | 2702 | CE1 | PHE | I | 146 | 38.740 | 41.731 | 27.971 | 1.00 | 19.18 | I | C |
| ATOM | 2703 | CE2 | PHE | I | 146 | 39.558 | 39.513 | 28.481 | 1.00 | 20.01 | I | C |
| ATOM | 2704 | CZ | PHE | I | 146 | 38.577 | 40.491 | 28.602 | 1.00 | 25.18 | I | C |
| ATOM | 2705 | C | PHE | I | 146 | 40.833 | 41.767 | 24.235 | 1.00 | 25.42 | I | C |
| ATOM | 2706 | O | PHE | I | 146 | 41.111 | 42.909 | 23.865 | 1.00 | 25.45 | I | O |
| ATOM | 2707 | N | PRO | I | 147 | 39.615 | 41.231 | 24.062 | 1.00 | 26.89 | I | N |
| ATOM | 2708 | CD | PRO | I | 147 | 38.380 | 41.995 | 23.783 | 1.00 | 27.16 | I | C |
| ATOM | 2709 | CA | PRO | I | 147 | 39.295 | 39.841 | 24.403 | 1.00 | 27.12 | I | C |
| ATOM | 2710 | CB | PRO | I | 147 | 37.845 | 39.955 | 24.871 | 1.00 | 28.76 | I | C |
| ATOM | 2711 | CG | PRO | I | 147 | 37.286 | 40.919 | 23.838 | 1.00 | 24.94 | I | C |
| ATOM | 2712 | C | PRO | I | 147 | 39.369 | 38.972 | 23.146 | 1.00 | 27.73 | I | C |
| ATOM | 2713 | O | PRO | I | 147 | 39.857 | 39.407 | 22.103 | 1.00 | 28.17 | I | O |
| ATOM | 2714 | N | GLU | I | 148 | 38.863 | 37.748 | 23.250 | 1.00 | 30.21 | I | N |
| ATOM | 2715 | CA | GLU | I | 148 | 38.809 | 36.864 | 22.101 | 1.00 | 32.19 | I | C |
| ATOM | 2716 | CB | GLU | I | 148 | 38.482 | 35.442 | 22.540 | 1.00 | 28.16 | I | C |
| ATOM | 2717 | CG | GLU | I | 148 | 39.468 | 34.904 | 23.529 | 1.00 | 32.75 | I | C |
| ATOM | 2718 | CD | GLU | I | 148 | 39.749 | 33.447 | 23.316 | 1.00 | 40.85 | I | C |
| ATOM | 2719 | OE1 | GLU | I | 148 | 40.342 | 33.107 | 22.266 | 1.00 | 44.12 | I | O |
| ATOM | 2720 | OE2 | GLU | I | 148 | 39.374 | 32.642 | 24.197 | 1.00 | 44.69 | I | O |
| ATOM | 2721 | C | GLU | I | 148 | 37.693 | 37.399 | 21.201 | 1.00 | 30.38 | I | C |
| ATOM | 2722 | O | GLU | I | 148 | 36.789 | 38.084 | 21.672 | 1.00 | 34.67 | I | O |
| ATOM | 2723 | N | PRO | I | 149 | 37.732 | 37.080 | 19.901 | 1.00 | 32.51 | I | N |
| ATOM | 2724 | CD | PRO | I | 149 | 36.582 | 37.219 | 18.988 | 1.00 | 32.57 | I | C |
| ATOM | 2725 | CA | PRO | I | 149 | 38.774 | 36.253 | 19.295 | 1.00 | 33.64 | I | C |
| ATOM | 2726 | CB | PRO | I | 149 | 37.957 | 35.252 | 18.496 | 1.00 | 31.37 | I | C |
| ATOM | 2727 | CG | PRO | I | 149 | 36.861 | 36.156 | 17.903 | 1.00 | 34.73 | I | C |
| ATOM | 2728 | C | PRO | I | 149 | 39.708 | 37.041 | 18.385 | 1.00 | 36.65 | I | C |
| ATOM | 2729 | O | PRO | I | 149 | 39.607 | 38.266 | 18.262 | 1.00 | 44.09 | I | O |
| ATOM | 2730 | N | VAL | I | 150 | 40.594 | 36.304 | 17.726 | 1.00 | 41.32 | I | N |
| ATOM | 2731 | CA | VAL | I | 150 | 41.552 | 36.857 | 16.786 | 1.00 | 38.68 | I | C |
| ATOM | 2732 | CB | VAL | I | 150 | 42.955 | 36.947 | 17.420 | 1.00 | 37.83 | I | C |
| ATOM | 2733 | CG1 | VAL | I | 150 | 43.590 | 35.581 | 17.529 | 1.00 | 38.41 | I | C |
| ATOM | 2734 | CG2 | VAL | I | 150 | 43.822 | 37.875 | 16.627 | 1.00 | 46.86 | I | C |
| ATOM | 2735 | C | VAL | I | 150 | 41.543 | 35.881 | 15.611 | 1.00 | 39.70 | I | C |
| ATOM | 2736 | O | VAL | I | 150 | 41.449 | 34.669 | 15.809 | 1.00 | 46.68 | I | O |
| ATOM | 2737 | N | THR | I | 151 | 41.514 | 36.401 | 14.390 | 1.00 | 41.52 | I | N |
| ATOM | 2738 | CA | THR | I | 151 | 41.516 | 35.529 | 13.216 | 1.00 | 37.73 | I | C |
| ATOM | 2739 | CB | THR | I | 151 | 40.430 | 35.942 | 12.156 | 1.00 | 36.92 | I | C |
| ATOM | 2740 | OG1 | THR | I | 151 | 40.585 | 37.319 | 11.806 | 1.00 | 37.19 | I | O |
| ATOM | 2741 | CG2 | THR | I | 151 | 39.017 | 35.728 | 12.697 | 1.00 | 34.19 | I | C |
| ATOM | 2742 | C | THR | I | 151 | 42.897 | 35.538 | 12.572 | 1.00 | 39.61 | I | C |
| ATOM | 2743 | O | THR | I | 151 | 43.605 | 36.548 | 12.606 | 1.00 | 37.54 | I | O |
| ATOM | 2744 | N | VAL | I | 152 | 43.295 | 34.389 | 12.030 | 1.00 | 43.42 | I | N |
| ATOM | 2745 | CA | VAL | I | 152 | 44.589 | 34.258 | 11.360 | 1.00 | 38.01 | I | C |
| ATOM | 2746 | CB | VAL | I | 152 | 45.617 | 33.477 | 12.227 | 1.00 | 36.23 | I | C |
| ATOM | 2747 | CG1 | VAL | I | 152 | 46.957 | 33.354 | 11.490 | 1.00 | 34.55 | I | C |
| ATOM | 2748 | CG2 | VAL | I | 152 | 45.817 | 34.178 | 13.551 | 1.00 | 39.71 | I | C |
| ATOM | 2749 | C | VAL | I | 152 | 44.420 | 33.551 | 10.017 | 1.00 | 36.81 | I | C |
| ATOM | 2750 | O | VAL | I | 152 | 43.718 | 32.544 | 9.924 | 1.00 | 35.23 | I | O |
| ATOM | 2751 | N | SER | I | 153 | 45.049 | 34.114 | 8.987 | 1.00 | 36.84 | I | N |
| ATOM | 2752 | CA | SER | I | 153 | 45.025 | 33.574 | 7.632 | 1.00 | 38.82 | I | C |
| ATOM | 2753 | CB | SER | I | 153 | 44.102 | 34.409 | 6.738 | 1.00 | 37.99 | I | C |
| ATOM | 2754 | OG | SER | I | 153 | 42.842 | 34.615 | 7.359 | 1.00 | 53.61 | I | O |
| ATOM | 2755 | C | SER | I | 153 | 46.444 | 33.698 | 7.119 | 1.00 | 37.38 | I | C |
| ATOM | 2756 | O | SER | I | 153 | 47.242 | 34.434 | 7.686 | 1.00 | 39.36 | I | O |
| ATOM | 2757 | N | TRP | I | 154 | 46.769 | 32.957 | 6.070 | 1.00 | 38.90 | I | N |
| ATOM | 2758 | CA | TRP | I | 154 | 48.097 | 33.041 | 5.475 | 1.00 | 42.08 | I | C |
| ATOM | 2759 | CB | TRP | I | 154 | 48.879 | 31.736 | 5.687 | 1.00 | 39.14 | I | C |
| ATOM | 2760 | CG | TRP | I | 154 | 49.348 | 31.517 | 7.113 | 1.00 | 36.44 | I | C |
| ATOM | 2761 | CD2 | TRP | I | 154 | 50.623 | 31.869 | 7.666 | 1.00 | 30.14 | I | C |
| ATOM | 2762 | CE2 | TRP | I | 154 | 50.615 | 31.468 | 9.023 | 1.00 | 32.27 | I | C |
| ATOM | 2763 | CE3 | TRP | I | 154 | 51.771 | 32.489 | 7.149 | 1.00 | 31.57 | I | C |
| ATOM | 2764 | CD1 | TRP | I | 154 | 48.644 | 30.932 | 8.123 | 1.00 | 34.20 | I | C |
| ATOM | 2765 | NE1 | TRP | I | 154 | 49.395 | 30.895 | 9.272 | 1.00 | 30.30 | I | N |
| ATOM | 2766 | CZ2 | TRP | I | 154 | 51.715 | 31.664 | 9.878 | 1.00 | 27.64 | I | C |
| ATOM | 2767 | CZ3 | TRP | I | 154 | 52.870 | 32.688 | 7.996 | 1.00 | 32.51 | I | C |
| ATOM | 2768 | CH2 | TRP | I | 154 | 52.828 | 32.277 | 9.351 | 1.00 | 31.80 | I | C |
| ATOM | 2769 | C | TRP | I | 154 | 47.983 | 33.405 | 3.980 | 1.00 | 44.46 | I | C |
| ATOM | 2770 | O | TRP | I | 154 | 47.125 | 32.873 | 3.267 | 1.00 | 47.08 | I | O |
| ATOM | 2771 | N | ASN | I | 155 | 48.823 | 34.341 | 3.532 | 1.00 | 44.09 | I | N |
| ATOM | 2772 | CA | ASN | I | 155 | 48.835 | 34.817 | 2.143 | 1.00 | 43.95 | I | C |
| ATOM | 2773 | CB | ASN | I | 155 | 49.423 | 33.759 | 1.212 | 1.00 | 44.29 | I | C |
| ATOM | 2774 | CG | ASN | I | 155 | 50.825 | 33.383 | 1.593 | 1.00 | 42.68 | I | C |
| ATOM | 2775 | OD1 | ASN | I | 155 | 51.267 | 32.261 | 1.353 | 1.00 | 46.60 | I | O |
| ATOM | 2776 | ND2 | ASN | I | 155 | 51.534 | 34.317 | 2.211 | 1.00 | 43.10 | I | N |
| ATOM | 2777 | C | ASN | I | 155 | 47.440 | 35.216 | 1.678 | 1.00 | 45.72 | I | C |
| ATOM | 2778 | O | ASN | I | 155 | 46.936 | 34.716 | 0.669 | 1.00 | 36.18 | I | O |
| ATOM | 2779 | N | SER | I | 156 | 46.817 | 36.109 | 2.446 | 1.00 | 50.67 | I | N |
| ATOM | 2780 | CA | SER | I | 156 | 45.461 | 36.597 | 2.177 | 1.00 | 53.23 | I | C |
| ATOM | 2781 | CB | SER | I | 156 | 45.398 | 37.350 | 0.841 | 1.00 | 52.02 | I | C |
| ATOM | 2782 | OG | SER | I | 156 | 46.080 | 38.586 | 0.916 | 1.00 | 51.12 | I | O |
| ATOM | 2783 | C | SER | I | 156 | 44.398 | 35.498 | 2.188 | 1.00 | 52.47 | I | C |
| ATOM | 2784 | O | SER | I | 156 | 43.208 | 35.792 | 2.104 | 1.00 | 60.27 | I | O |
| ATOM | 2785 | N | GLY | I | 157 | 44.828 | 34.241 | 2.288 | 1.00 | 51.40 | I | N |
| ATOM | 2786 | CA | GLY | I | 157 | 43.903 | 33.122 | 2.291 | 1.00 | 47.47 | I | C |
| ATOM | 2787 | C | GLY | I | 157 | 44.313 | 32.066 | 1.283 | 1.00 | 50.80 | I | C |
| ATOM | 2788 | O | GLY | I | 157 | 43.781 | 30.957 | 1.280 | 1.00 | 50.70 | I | O |
| ATOM | 2789 | N | ALA | I | 158 | 45.289 | 32.403 | 0.444 | 1.00 | 49.27 | I | N |
| ATOM | 2790 | CA | ALA | I | 158 | 45.786 | 31.498 | −0.592 | 1.00 | 49.16 | I | C |
| ATOM | 2791 | CB | ALA | I | 158 | 46.528 | 32.291 | −1.663 | 1.00 | 43.11 | I | C |
| ATOM | 2792 | C | ALA | I | 158 | 46.674 | 30.365 | −0.073 | 1.00 | 51.69 | I | C |
| ATOM | 2793 | O | ALA | I | 158 | 47.359 | 29.705 | −0.860 | 1.00 | 56.52 | I | O |
| ATOM | 2794 | N | LEU | I | 159 | 46.679 | 30.150 | 1.241 | 1.00 | 46.92 | I | N |
| ATOM | 2795 | CA | LEU | I | 159 | 47.485 | 29.090 | 1.838 | 1.00 | 42.59 | I | C |
| ATOM | 2796 | CB | LEU | I | 159 | 48.859 | 29.626 | 2.272 | 1.00 | 36.80 | I | C |
| ATOM | 2797 | CG | LEU | I | 159 | 49.868 | 28.632 | 2.877 | 1.00 | 39.01 | I | C |
| ATOM | 2798 | CD1 | LEU | I | 159 | 50.185 | 27.534 | 1.887 | 1.00 | 36.23 | I | C |
| ATOM | 2799 | CD2 | LEU | I | 159 | 51.160 | 29.332 | 3.293 | 1.00 | 32.37 | I | C |
| ATOM | 2800 | C | LEU | I | 159 | 46.705 | 28.511 | 3.018 | 1.00 | 44.97 | I | C |
| ATOM | 2801 | O | LEU | I | 159 | 46.471 | 29.189 | 4.022 | 1.00 | 44.34 | I | O |
| ATOM | 2802 | N | THR | I | 160 | 46.253 | 27.270 | 2.855 | 1.00 | 45.73 | I | N |
| ATOM | 2803 | CA | THR | I | 160 | 45.470 | 26.593 | 3.876 | 1.00 | 43.55 | I | C |
| ATOM | 2804 | CB | THR | I | 160 | 44.020 | 26.356 | 3.396 | 1.00 | 52.02 | I | C |
| ATOM | 2805 | OG1 | THR | I | 160 | 44.036 | 25.745 | 2.092 | 1.00 | 52.31 | I | O |
| ATOM | 2806 | CG2 | THR | I | 160 | 43.234 | 27.673 | 3.366 | 1.00 | 46.71 | I | C |
| ATOM | 2807 | C | THR | I | 160 | 46.058 | 25.247 | 4.272 | 1.00 | 46.99 | I | C |
| ATOM | 2808 | O | THR | I | 160 | 45.940 | 24.820 | 5.428 | 1.00 | 49.07 | I | O |
| ATOM | 2809 | N | SER | I | 161 | 46.673 | 24.563 | 3.314 | 1.00 | 42.15 | I | N |
| ATOM | 2810 | CA | SER | I | 161 | 47.257 | 23.264 | 3.604 | 1.00 | 44.96 | I | C |
| ATOM | 2811 | CB | SER | I | 161 | 47.716 | 22.560 | 2.328 | 1.00 | 41.38 | I | C |
| ATOM | 2812 | OG | SER | I | 161 | 46.594 | 22.144 | 1.576 | 1.00 | 52.28 | I | O |
| ATOM | 2813 | C | SER | I | 161 | 48.415 | 23.373 | 4.565 | 1.00 | 42.51 | I | C |
| ATOM | 2814 | O | SER | I | 161 | 49.262 | 24.257 | 4.428 | 1.00 | 47.34 | I | O |
| ATOM | 2815 | N | GLY | I | 162 | 48.444 | 22.450 | 5.526 | 1.00 | 42.82 | I | N |
| ATOM | 2816 | CA | GLY | I | 162 | 49.502 | 22.403 | 6.520 | 1.00 | 36.35 | I | C |
| ATOM | 2817 | C | GLY | I | 162 | 49.408 | 23.457 | 7.608 | 1.00 | 37.30 | I | C |
| ATOM | 2818 | O | GLY | I | 162 | 50.294 | 23.533 | 8.463 | 1.00 | 33.08 | I | O |
| ATOM | 2819 | N | VAL | I | 163 | 48.345 | 24.262 | 7.564 | 1.00 | 26.35 | I | N |
| ATOM | 2820 | CA | VAL | I | 163 | 48.123 | 25.324 | 8.529 | 1.00 | 30.92 | I | C |
| ATOM | 2821 | CB | VAL | I | 163 | 47.288 | 26.479 | 7.911 | 1.00 | 30.16 | I | C |
| ATOM | 2822 | CG1 | VAL | I | 163 | 46.635 | 27.321 | 9.008 | 1.00 | 23.71 | I | C |
| ATOM | 2823 | CG2 | VAL | I | 163 | 48.175 | 27.361 | 7.051 | 1.00 | 27.02 | I | C |
| ATOM | 2824 | C | VAL | I | 163 | 47.437 | 24.844 | 9.799 | 1.00 | 30.83 | I | C |
| ATOM | 2825 | O | VAL | I | 163 | 46.431 | 24.135 | 9.744 | 1.00 | 29.58 | I | O |
| ATOM | 2826 | N | HIS | I | 164 | 48.004 | 25.242 | 10.938 | 1.00 | 35.98 | I | N |
| ATOM | 2827 | CA | HIS | I | 164 | 47.477 | 24.906 | 12.255 | 1.00 | 29.84 | I | C |
| ATOM | 2828 | CB | HIS | I | 164 | 48.359 | 23.869 | 12.910 | 1.00 | 31.38 | I | C |
| ATOM | 2829 | CG | HIS | I | 164 | 48.153 | 22.496 | 12.368 | 1.00 | 36.61 | I | C |
| ATOM | 2830 | CD2 | HIS | I | 164 | 47.034 | 21.744 | 12.264 | 1.00 | 31.07 | I | C |
| ATOM | 2831 | ND1 | HIS | I | 164 | 49.184 | 21.726 | 11.875 | 1.00 | 35.82 | I | N |
| ATOM | 2832 | CE1 | HIS | I | 164 | 48.709 | 20.555 | 11.498 | 1.00 | 31.29 | I | C |
| ATOM | 2833 | NE2 | HIS | I | 164 | 47.409 | 20.541 | 11.724 | 1.00 | 31.77 | I | N |
| ATOM | 2834 | C | HIS | I | 164 | 47.408 | 26.137 | 13.137 | 1.00 | 31.35 | I | C |
| ATOM | 2835 | O | HIS | I | 164 | 48.435 | 26.694 | 13.520 | 1.00 | 33.64 | I | O |
| ATOM | 2836 | N | THR | I | 165 | 46.196 | 26.618 | 13.370 | 1.00 | 26.18 | I | N |
| ATOM | 2837 | CA | THR | I | 165 | 45.995 | 27.779 | 14.220 | 1.00 | 26.50 | I | C |
| ATOM | 2838 | CB | THR | I | 165 | 44.992 | 28.781 | 13.604 | 1.00 | 28.35 | I | C |
| ATOM | 2839 | OG1 | THR | I | 165 | 45.531 | 29.304 | 12.394 | 1.00 | 27.86 | I | O |
| ATOM | 2840 | CG2 | THR | I | 165 | 44.729 | 29.937 | 14.542 | 1.00 | 21.91 | I | C |
| ATOM | 2841 | C | THR | I | 165 | 45.485 | 27.225 | 15.548 | 1.00 | 29.12 | I | C |
| ATOM | 2842 | O | THR | I | 165 | 44.320 | 26.809 | 15.675 | 1.00 | 27.23 | I | O |
| ATOM | 2843 | N | PHE | I | 166 | 46.395 | 27.171 | 16.516 | 1.00 | 24.05 | I | N |
| ATOM | 2844 | CA | PHE | I | 166 | 46.108 | 26.645 | 17.843 | 1.00 | 22.54 | I | C |
| ATOM | 2845 | CB | PHE | I | 166 | 47.411 | 26.571 | 18.649 | 1.00 | 21.78 | I | C |
| ATOM | 2846 | CG | PHE | I | 166 | 48.432 | 25.617 | 18.081 | 1.00 | 25.60 | I | C |
| ATOM | 2847 | CD1 | PHE | I | 166 | 48.384 | 24.257 | 18.389 | 1.00 | 25.14 | I | C |
| ATOM | 2848 | CD2 | PHE | I | 166 | 49.443 | 26.074 | 17.242 | 1.00 | 23.73 | I | C |
| ATOM | 2849 | CE1 | PHE | I | 166 | 49.329 | 23.372 | 17.867 | 1.00 | 22.75 | I | C |
| ATOM | 2850 | CE2 | PHE | I | 166 | 50.381 | 25.200 | 16.720 | 1.00 | 20.41 | I | C |
| ATOM | 2851 | CZ | PHE | I | 166 | 50.322 | 23.844 | 17.035 | 1.00 | 21.72 | I | C |
| ATOM | 2852 | C | PHE | I | 166 | 45.078 | 27.429 | 18.648 | 1.00 | 21.45 | I | C |
| ATOM | 2853 | O | PHE | I | 166 | 44.903 | 28.629 | 18.464 | 1.00 | 23.16 | I | O |
| ATOM | 2854 | N | PRO | I | 167 | 44.374 | 26.750 | 19.556 | 1.00 | 19.69 | I | N |
| ATOM | 2855 | CD | PRO | I | 167 | 44.394 | 25.304 | 19.832 | 1.00 | 25.23 | I | C |
| ATOM | 2856 | CA | PRO | I | 167 | 43.380 | 27.428 | 20.386 | 1.00 | 24.43 | I | C |
| ATOM | 2857 | CB | PRO | I | 167 | 42.817 | 26.282 | 21.241 | 1.00 | 23.63 | I | C |
| ATOM | 2858 | CG | PRO | I | 167 | 43.034 | 25.071 | 20.414 | 1.00 | 18.01 | I | C |
| ATOM | 2859 | C | PRO | I | 167 | 44.149 | 28.410 | 21.286 | 1.00 | 27.83 | I | C |
| ATOM | 2860 | O | PRO | I | 167 | 45.273 | 28.135 | 21.689 | 1.00 | 25.38 | I | O |
| ATOM | 2861 | N | ALA | I | 168 | 43.545 | 29.540 | 21.614 | 1.00 | 31.85 | I | N |
| ATOM | 2862 | CA | ALA | I | 168 | 44.207 | 30.514 | 22.467 | 1.00 | 26.87 | I | C |
| ATOM | 2863 | CB | ALA | I | 168 | 43.503 | 31.849 | 22.368 | 1.00 | 26.17 | I | C |
| ATOM | 2864 | C | ALA | I | 168 | 44.231 | 30.059 | 23.914 | 1.00 | 29.50 | I | C |
| ATOM | 2865 | O | ALA | I | 168 | 43.452 | 29.195 | 24.317 | 1.00 | 30.25 | I | O |
| ATOM | 2866 | N | VAL | I | 169 | 45.177 | 30.598 | 24.679 | 1.00 | 29.26 | I | N |
| ATOM | 2867 | CA | VAL | I | 169 | 45.258 | 30.289 | 26.103 | 1.00 | 27.59 | I | C |
| ATOM | 2868 | CB | VAL | I | 169 | 46.504 | 29.466 | 26.491 | 1.00 | 27.53 | I | C |
| ATOM | 2869 | CG1 | VAL | I | 169 | 46.675 | 28.292 | 25.533 | 1.00 | 26.24 | I | C |
| ATOM | 2870 | CG2 | VAL | I | 169 | 47.757 | 30.355 | 26.572 | 1.00 | 33.69 | I | C |
| ATOM | 2871 | C | VAL | I | 169 | 45.244 | 31.590 | 26.878 | 1.00 | 22.61 | I | C |
| ATOM | 2872 | O | VAL | I | 169 | 45.673 | 32.626 | 26.370 | 1.00 | 24.86 | I | O |
| ATOM | 2873 | N | LEU | I | 170 | 44.687 | 31.530 | 28.081 | 1.00 | 27.32 | I | N |
| ATOM | 2874 | CA | LEU | I | 170 | 44.599 | 32.676 | 28.970 | 1.00 | 26.05 | I | C |
| ATOM | 2875 | CB | LEU | I | 170 | 43.352 | 32.560 | 29.852 | 1.00 | 23.68 | I | C |
| ATOM | 2876 | CG | LEU | I | 170 | 43.058 | 33.670 | 30.882 | 1.00 | 29.33 | I | C |
| ATOM | 2877 | CD1 | LEU | I | 170 | 42.976 | 35.048 | 30.214 | 1.00 | 17.88 | I | C |
| ATOM | 2878 | CD2 | LEU | I | 170 | 41.724 | 33.351 | 31.596 | 1.00 | 22.36 | I | C |
| ATOM | 2879 | C | LEU | I | 170 | 45.836 | 32.698 | 29.855 | 1.00 | 26.19 | I | C |
| ATOM | 2880 | O | LEU | I | 170 | 46.070 | 31.756 | 30.606 | 1.00 | 26.57 | I | O |
| ATOM | 2881 | N | GLN | I | 171 | 46.630 | 33.759 | 29.741 | 1.00 | 24.48 | I | N |
| ATOM | 2882 | CA | GLN | I | 171 | 47.838 | 33.928 | 30.555 | 1.00 | 32.32 | I | C |
| ATOM | 2883 | CB | GLN | I | 171 | 48.835 | 34.825 | 29.831 | 1.00 | 26.87 | I | C |
| ATOM | 2884 | CG | GLN | I | 171 | 49.454 | 34.102 | 28.675 | 1.00 | 34.83 | I | C |
| ATOM | 2885 | CD | GLN | I | 171 | 50.014 | 35.024 | 27.651 | 1.00 | 36.61 | I | C |
| ATOM | 2886 | OE1 | GLN | I | 171 | 51.222 | 35.020 | 27.400 | 1.00 | 40.19 | I | O |
| ATOM | 2887 | NE2 | GLN | I | 171 | 49.142 | 35.821 | 27.025 | 1.00 | 37.70 | I | N |
| ATOM | 2888 | C | GLN | I | 171 | 47.502 | 34.528 | 31.904 | 1.00 | 33.43 | I | C |
| ATOM | 2889 | O | GLN | I | 171 | 46.449 | 35.149 | 32.061 | 1.00 | 37.77 | I | O |
| ATOM | 2890 | N | SER | I | 172 | 48.397 | 34.363 | 32.876 | 1.00 | 34.14 | I | N |
| ATOM | 2891 | CA | SER | I | 172 | 48.158 | 34.911 | 34.209 | 1.00 | 31.78 | I | C |
| ATOM | 2892 | CB | SER | I | 172 | 49.305 | 34.545 | 35.158 | 1.00 | 35.21 | I | C |
| ATOM | 2893 | OG | SER | I | 172 | 50.543 | 35.047 | 34.693 | 1.00 | 35.85 | I | O |
| ATOM | 2894 | C | SER | I | 172 | 47.951 | 36.430 | 34.188 | 1.00 | 28.56 | I | C |
| ATOM | 2895 | O | SER | I | 172 | 47.482 | 37.005 | 35.165 | 1.00 | 29.37 | I | O |
| ATOM | 2896 | N | SER | I | 173 | 48.285 | 37.067 | 33.065 | 1.00 | 26.43 | I | N |
| ATOM | 2897 | CA | SER | I | 173 | 48.128 | 38.516 | 32.904 | 1.00 | 24.46 | I | C |
| ATOM | 2898 | CB | SER | I | 173 | 49.046 | 39.017 | 31.786 | 1.00 | 23.02 | I | C |
| ATOM | 2899 | OG | SER | I | 173 | 48.688 | 38.454 | 30.532 | 1.00 | 24.60 | I | O |
| ATOM | 2900 | C | SER | I | 173 | 46.692 | 38.920 | 32.564 | 1.00 | 27.06 | I | C |
| ATOM | 2901 | O | SER | I | 173 | 46.361 | 40.100 | 32.577 | 1.00 | 24.06 | I | O |
| ATOM | 2902 | N | GLY | I | 174 | 45.865 | 37.931 | 32.220 | 1.00 | 26.86 | I | N |
| ATOM | 2903 | CA | GLY | I | 174 | 44.486 | 38.186 | 31.841 | 1.00 | 26.30 | I | C |
| ATOM | 2904 | C | GLY | I | 174 | 44.336 | 38.425 | 30.346 | 1.00 | 24.81 | I | C |
| ATOM | 2905 | O | GLY | I | 174 | 43.233 | 38.687 | 29.854 | 1.00 | 24.09 | I | O |
| ATOM | 2906 | N | LEU | I | 175 | 45.451 | 38.308 | 29.629 | 1.00 | 21.78 | I | N |
| ATOM | 2907 | CA | LEU | I | 175 | 45.491 | 38.520 | 28.184 | 1.00 | 22.47 | I | C |
| ATOM | 2908 | CB | LEU | I | 175 | 46.643 | 39.477 | 27.822 | 1.00 | 21.86 | I | C |
| ATOM | 2909 | CG | LEU | I | 175 | 46.696 | 40.878 | 28.482 | 1.00 | 24.76 | I | C |
| ATOM | 2910 | CD1 | LEU | I | 175 | 47.949 | 41.648 | 28.030 | 1.00 | 14.15 | I | C |
| ATOM | 2911 | CD2 | LEU | I | 175 | 45.442 | 41.683 | 28.144 | 1.00 | 11.08 | I | C |
| ATOM | 2912 | C | LEU | I | 175 | 45.652 | 37.187 | 27.454 | 1.00 | 21.98 | I | C |
| ATOM | 2913 | O | LEU | I | 175 | 46.161 | 36.223 | 28.012 | 1.00 | 19.75 | I | O |
| ATOM | 2914 | N | TYR | I | 176 | 45.208 | 37.133 | 26.206 | 1.00 | 22.94 | I | N |
| ATOM | 2915 | CA | TYR | I | 176 | 45.293 | 35.907 | 25.435 | 1.00 | 22.56 | I | C |
| ATOM | 2916 | CB | TYR | I | 176 | 43.985 | 35.672 | 24.668 | 1.00 | 26.08 | I | C |
| ATOM | 2917 | CG | TYR | I | 176 | 42.784 | 35.339 | 25.539 | 1.00 | 25.50 | I | C |
| ATOM | 2918 | CD1 | TYR | I | 176 | 42.423 | 34.011 | 25.803 | 1.00 | 25.27 | I | C |
| ATOM | 2919 | CE1 | TYR | I | 176 | 41.265 | 33.702 | 26.547 | 1.00 | 18.30 | I | C |
| ATOM | 2920 | CD2 | TYR | I | 176 | 41.969 | 36.351 | 26.050 | 1.00 | 28.00 | I | C |
| ATOM | 2921 | CE2 | TYR | I | 176 | 40.816 | 36.054 | 26.798 | 1.00 | 24.83 | I | C |
| ATOM | 2922 | CZ | TYR | I | 176 | 40.466 | 34.735 | 27.036 | 1.00 | 25.52 | I | C |
| ATOM | 2923 | OH | TYR | I | 176 | 39.290 | 34.476 | 27.724 | 1.00 | 30.42 | I | O |
| ATOM | 2924 | C | TYR | I | 176 | 46.486 | 35.840 | 24.487 | 1.00 | 24.75 | I | C |
| ATOM | 2925 | O | TYR | I | 176 | 46.996 | 36.856 | 23.997 | 1.00 | 25.77 | I | O |
| ATOM | 2926 | N | SER | I | 177 | 46.923 | 34.617 | 24.233 | 1.00 | 22.63 | I | N |
| ATOM | 2927 | CA | SER | I | 177 | 48.051 | 34.366 | 23.343 | 1.00 | 25.09 | I | C |
| ATOM | 2928 | CB | SER | I | 177 | 49.323 | 34.143 | 24.155 | 1.00 | 17.87 | I | C |
| ATOM | 2929 | OG | SER | I | 177 | 50.191 | 33.221 | 23.528 | 1.00 | 39.90 | I | O |
| ATOM | 2930 | C | SER | I | 177 | 47.722 | 33.154 | 22.464 | 1.00 | 26.81 | I | C |
| ATOM | 2931 | O | SER | I | 177 | 47.059 | 32.202 | 22.889 | 1.00 | 22.24 | I | O |
| ATOM | 2932 | N | LEU | I | 178 | 48.227 | 33.182 | 21.245 | 1.00 | 25.44 | I | N |
| ATOM | 2933 | CA | LEU | I | 178 | 47.951 | 32.137 | 20.294 | 1.00 | 24.88 | I | C |
| ATOM | 2934 | CB | LEU | I | 178 | 46.596 | 32.457 | 19.650 | 1.00 | 22.52 | I | C |
| ATOM | 2935 | CG | LEU | I | 178 | 46.108 | 31.942 | 18.304 | 1.00 | 24.02 | I | C |
| ATOM | 2936 | CD1 | LEU | I | 178 | 44.583 | 32.072 | 18.262 | 1.00 | 28.12 | I | C |
| ATOM | 2937 | CD2 | LEU | I | 178 | 46.730 | 32.716 | 17.155 | 1.00 | 20.60 | I | C |
| ATOM | 2938 | C | LEU | I | 178 | 49.041 | 32.058 | 19.241 | 1.00 | 25.79 | I | C |
| ATOM | 2939 | O | LEU | I | 178 | 49.690 | 33.050 | 18.914 | 1.00 | 26.92 | I | O |
| ATOM | 2940 | N | SER | I | 179 | 49.181 | 30.884 | 18.652 | 1.00 | 28.80 | I | N |
| ATOM | 2941 | CA | SER | I | 179 | 50.172 | 30.684 | 17.629 | 1.00 | 26.83 | I | C |
| ATOM | 2942 | CB | SER | I | 179 | 51.306 | 29.837 | 18.204 | 1.00 | 34.12 | I | C |
| ATOM | 2943 | OG | SER | I | 179 | 52.411 | 29.803 | 17.323 | 1.00 | 41.53 | I | O |
| ATOM | 2944 | C | SER | I | 179 | 49.560 | 29.991 | 16.415 | 1.00 | 28.01 | I | C |
| ATOM | 2945 | O | SER | I | 179 | 48.709 | 29.121 | 16.554 | 1.00 | 18.28 | I | O |
| ATOM | 2946 | N | SER | I | 180 | 49.974 | 30.412 | 15.225 | 1.00 | 25.69 | I | N |
| ATOM | 2947 | CA | SER | I | 180 | 49.506 | 29.797 | 13.990 | 1.00 | 24.93 | I | C |
| ATOM | 2948 | CB | SER | I | 180 | 48.647 | 30.753 | 13.159 | 1.00 | 25.33 | I | C |
| ATOM | 2949 | OG | SER | I | 180 | 48.272 | 30.136 | 11.932 | 1.00 | 22.85 | I | O |
| ATOM | 2950 | C | SER | I | 180 | 50.754 | 29.405 | 13.219 | 1.00 | 25.46 | I | C |
| ATOM | 2951 | O | SER | I | 180 | 51.608 | 30.229 | 12.941 | 1.00 | 28.87 | I | O |
| ATOM | 2952 | N | VAL | I | 181 | 50.864 | 28.132 | 12.894 | 1.00 | 29.99 | I | N |
| ATOM | 2953 | CA | VAL | I | 181 | 52.017 | 27.641 | 12.178 | 1.00 | 25.74 | I | C |
| ATOM | 2954 | CB | VAL | I | 181 | 52.751 | 26.580 | 13.006 | 1.00 | 27.11 | I | C |
| ATOM | 2955 | CG1 | VAL | I | 181 | 53.252 | 27.181 | 14.296 | 1.00 | 22.24 | I | C |
| ATOM | 2956 | CG2 | VAL | I | 181 | 51.830 | 25.417 | 13.296 | 1.00 | 25.57 | I | C |
| ATOM | 2957 | C | VAL | I | 181 | 51.623 | 27.023 | 10.851 | 1.00 | 34.53 | I | C |
| ATOM | 2958 | O | VAL | I | 181 | 50.445 | 26.708 | 10.597 | 1.00 | 36.36 | I | O |
| ATOM | 2959 | N | VAL | I | 182 | 52.627 | 26.848 | 10.002 | 1.00 | 36.80 | I | N |
| ATOM | 2960 | CA | VAL | I | 182 | 52.433 | 26.238 | 8.701 | 1.00 | 33.79 | I | C |
| ATOM | 2961 | CB | VAL | I | 182 | 52.071 | 27.295 | 7.643 | 1.00 | 32.92 | I | C |
| ATOM | 2962 | CG1 | VAL | I | 182 | 53.201 | 28.296 | 7.473 | 1.00 | 31.37 | I | C |
| ATOM | 2963 | CG2 | VAL | I | 182 | 51.700 | 26.614 | 6.323 | 1.00 | 30.80 | I | C |
| ATOM | 2964 | C | VAL | I | 182 | 53.700 | 25.496 | 8.295 | 1.00 | 34.49 | I | C |
| ATOM | 2965 | O | VAL | I | 182 | 54.801 | 25.903 | 8.664 | 1.00 | 37.04 | I | O |
| ATOM | 2966 | N | THR | I | 183 | 53.535 | 24.324 | 7.687 | 1.00 | 39.50 | I | N |
| ATOM | 2967 | CA | THR | I | 183 | 54.678 | 23.560 | 7.200 | 1.00 | 38.68 | I | C |
| ATOM | 2968 | CB | THR | I | 183 | 54.682 | 22.094 | 7.650 | 1.00 | 37.23 | I | C |
| ATOM | 2969 | OG1 | THR | I | 183 | 53.440 | 21.469 | 7.303 | 1.00 | 41.89 | I | O |
| ATOM | 2970 | CG2 | THR | I | 183 | 54.924 | 22.001 | 9.136 | 1.00 | 33.29 | I | C |
| ATOM | 2971 | C | THR | I | 183 | 54.663 | 23.663 | 5.679 | 1.00 | 42.44 | I | C |
| ATOM | 2972 | O | THR | I | 183 | 53.625 | 23.471 | 5.032 | 1.00 | 38.92 | I | O |
| ATOM | 2973 | N | VAL | I | 184 | 55.809 | 24.062 | 5.134 | 1.00 | 47.88 | I | N |
| ATOM | 2974 | CA | VAL | I | 184 | 55.995 | 24.260 | 3.697 | 1.00 | 46.23 | I | C |
| ATOM | 2975 | CB | VAL | I | 184 | 56.132 | 25.782 | 3.377 | 1.00 | 48.22 | I | C |
| ATOM | 2976 | CG1 | VAL | I | 184 | 54.898 | 26.544 | 3.843 | 1.00 | 45.36 | I | C |
| ATOM | 2977 | CG2 | VAL | I | 184 | 57.389 | 26.355 | 4.031 | 1.00 | 40.70 | I | C |
| ATOM | 2978 | C | VAL | I | 184 | 57.281 | 23.576 | 3.236 | 1.00 | 47.51 | I | C |
| ATOM | 2979 | O | VAL | I | 184 | 58.117 | 23.185 | 4.062 | 1.00 | 50.68 | I | O |
| ATOM | 2980 | N | PRO | I | 185 | 57.465 | 23.428 | 1.910 | 1.00 | 46.73 | I | N |
| ATOM | 2981 | CD | PRO | I | 185 | 56.569 | 23.811 | 0.802 | 1.00 | 48.11 | I | C |
| ATOM | 2982 | CA | PRO | I | 185 | 58.687 | 22.788 | 1.400 | 1.00 | 46.93 | I | C |
| ATOM | 2983 | CB | PRO | I | 185 | 58.465 | 22.788 | −0.121 | 1.00 | 46.90 | I | C |
| ATOM | 2984 | CG | PRO | I | 185 | 56.963 | 22.836 | −0.278 | 1.00 | 48.08 | I | C |
| ATOM | 2985 | C | PRO | I | 185 | 59.896 | 23.673 | 1.758 | 1.00 | 49.57 | I | C |
| ATOM | 2986 | O | PRO | I | 185 | 59.799 | 24.902 | 1.692 | 1.00 | 53.48 | I | O |
| ATOM | 2987 | N | SER | I | 186 | 61.016 | 23.072 | 2.151 | 1.00 | 47.79 | I | N |
| ATOM | 2988 | CA | SER | I | 186 | 62.207 | 23.855 | 2.492 | 1.00 | 52.81 | I | C |
| ATOM | 2989 | CB | SER | I | 186 | 63.280 | 22.952 | 3.109 | 1.00 | 52.52 | I | C |
| ATOM | 2990 | OG | SER | I | 186 | 62.807 | 22.314 | 4.284 | 1.00 | 52.15 | I | O |
| ATOM | 2991 | C | SER | I | 186 | 62.794 | 24.626 | 1.291 | 1.00 | 57.03 | I | C |
| ATOM | 2992 | O | SER | I | 186 | 63.454 | 25.654 | 1.462 | 1.00 | 60.15 | I | O |
| ATOM | 2993 | N | SER | I | 187 | 62.535 | 24.134 | 0.082 | 1.00 | 58.33 | I | N |
| ATOM | 2994 | CA | SER | I | 187 | 63.036 | 24.759 | −1.137 | 1.00 | 61.54 | I | C |
| ATOM | 2995 | CB | SER | I | 187 | 62.822 | 23.816 | −2.325 | 1.00 | 61.86 | I | C |
| ATOM | 2996 | OG | SER | I | 187 | 61.468 | 23.401 | −2.418 | 1.00 | 66.56 | I | O |
| ATOM | 2997 | C | SER | I | 187 | 62.421 | 26.134 | −1.433 | 1.00 | 63.47 | I | C |
| ATOM | 2998 | O | SER | I | 187 | 63.107 | 27.031 | −1.948 | 1.00 | 65.49 | I | O |
| ATOM | 2999 | N | SER | I | 188 | 61.144 | 26.307 | −1.086 | 1.00 | 58.73 | I | N |
| ATOM | 3000 | CA | SER | I | 188 | 60.441 | 27.568 | −1.331 | 1.00 | 57.59 | I | C |
| ATOM | 3001 | CB | SER | I | 188 | 58.924 | 27.364 | −1.270 | 1.00 | 55.09 | I | C |
| ATOM | 3002 | OG | SER | I | 188 | 58.511 | 27.003 | 0.035 | 1.00 | 54.57 | I | O |
| ATOM | 3003 | C | SER | I | 188 | 60.844 | 28.739 | −0.425 | 1.00 | 59.35 | I | C |
| ATOM | 3004 | O | SER | I | 188 | 60.347 | 29.858 | −0.606 | 1.00 | 62.63 | I | O |
| ATOM | 3005 | N | LEU | I | 189 | 61.736 | 28.489 | 0.536 | 1.00 | 56.45 | I | N |
| ATOM | 3006 | CA | LEU | I | 189 | 62.197 | 29.535 | 1.453 | 1.00 | 55.43 | I | C |
| ATOM | 3007 | CB | LEU | I | 189 | 62.968 | 28.924 | 2.626 | 1.00 | 48.13 | I | C |
| ATOM | 3008 | CG | LEU | I | 189 | 62.121 | 27.981 | 3.481 | 1.00 | 45.49 | I | C |
| ATOM | 3009 | CD1 | LEU | I | 189 | 62.878 | 27.574 | 4.738 | 1.00 | 45.35 | I | C |
| ATOM | 3010 | CD2 | LEU | I | 189 | 60.811 | 28.668 | 3.851 | 1.00 | 45.66 | I | C |
| ATOM | 3011 | C | LEU | I | 189 | 63.042 | 30.578 | 0.723 | 1.00 | 57.47 | I | C |
| ATOM | 3012 | O | LEU | I | 189 | 64.257 | 30.430 | 0.558 | 1.00 | 61.16 | I | O |
| ATOM | 3013 | N | GLY | I | 190 | 62.367 | 31.640 | 0.298 | 1.00 | 59.69 | I | N |
| ATOM | 3014 | CA | GLY | I | 190 | 62.999 | 32.713 | −0.450 | 1.00 | 63.34 | I | C |
| ATOM | 3015 | C | GLY | I | 190 | 62.139 | 32.973 | −1.681 | 1.00 | 64.25 | I | C |
| ATOM | 3016 | O | GLY | I | 190 | 61.653 | 34.090 | −1.907 | 1.00 | 61.09 | I | O |
| ATOM | 3017 | N | THR | I | 191 | 61.902 | 31.914 | −2.449 | 1.00 | 62.92 | I | N |
| ATOM | 3018 | CA | THR | I | 191 | 61.089 | 32.005 | −3.654 | 1.00 | 63.64 | I | C |
| ATOM | 3019 | CB | THR | I | 191 | 61.105 | 30.669 | −4.424 | 1.00 | 62.36 | I | C |
| ATOM | 3020 | OG1 | THR | I | 191 | 62.462 | 30.231 | −4.583 | 1.00 | 54.17 | I | O |
| ATOM | 3021 | CG2 | THR | I | 191 | 60.459 | 30.840 | −5.805 | 1.00 | 65.03 | I | C |
| ATOM | 3022 | C | THR | I | 191 | 59.640 | 32.409 | −3.325 | 1.00 | 64.38 | I | C |
| ATOM | 3023 | O | THR | I | 191 | 59.091 | 33.339 | −3.933 | 1.00 | 63.13 | I | O |
| ATOM | 3024 | N | GLN | I | 192 | 59.041 | 31.725 | −2.346 | 1.00 | 62.70 | I | N |
| ATOM | 3025 | CA | GLN | I | 192 | 57.666 | 32.003 | −1.932 | 1.00 | 58.25 | I | C |
| ATOM | 3026 | CB | GLN | I | 192 | 56.932 | 30.696 | −1.632 | 1.00 | 57.00 | I | C |
| ATOM | 3027 | CG | GLN | I | 192 | 55.489 | 30.878 | −1.194 | 1.00 | 59.87 | I | C |
| ATOM | 3028 | CD | GLN | I | 192 | 54.633 | 31.608 | −2.222 | 1.00 | 63.49 | I | C |
| ATOM | 3029 | OE1 | GLN | I | 192 | 54.578 | 32.841 | −2.240 | 1.00 | 63.44 | I | O |
| ATOM | 3030 | NE2 | GLN | I | 192 | 53.944 | 30.846 | −3.073 | 1.00 | 62.27 | I | N |
| ATOM | 3031 | C | GLN | I | 192 | 57.615 | 32.918 | −0.713 | 1.00 | 56.43 | I | C |
| ATOM | 3032 | O | GLN | I | 192 | 58.410 | 32.773 | 0.208 | 1.00 | 53.92 | I | O |
| ATOM | 3033 | N | THR | I | 193 | 56.684 | 33.867 | −0.725 | 1.00 | 56.91 | I | N |
| ATOM | 3034 | CA | THR | I | 193 | 56.511 | 34.806 | 0.380 | 1.00 | 53.62 | I | C |
| ATOM | 3035 | CB | THR | I | 193 | 56.410 | 36.261 | −0.145 | 1.00 | 52.32 | I | C |
| ATOM | 3036 | OG1 | THR | I | 193 | 57.732 | 36.802 | −0.264 | 1.00 | 55.24 | I | O |
| ATOM | 3037 | CG2 | THR | I | 193 | 55.600 | 37.143 | 0.782 | 1.00 | 54.13 | I | C |
| ATOM | 3038 | C | THR | I | 193 | 55.318 | 34.428 | 1.280 | 1.00 | 53.69 | I | C |
| ATOM | 3039 | O | THR | I | 193 | 54.195 | 34.245 | 0.802 | 1.00 | 51.28 | I | O |
| ATOM | 3040 | N | TYR | I | 194 | 55.591 | 34.266 | 2.578 | 1.00 | 49.65 | I | N |
| ATOM | 3041 | CA | TYR | I | 194 | 54.560 | 33.904 | 3.549 | 1.00 | 40.84 | I | C |
| ATOM | 3042 | CB | TYR | I | 194 | 55.001 | 32.710 | 4.369 | 1.00 | 39.38 | I | C |
| ATOM | 3043 | CG | TYR | I | 194 | 55.359 | 31.519 | 3.527 | 1.00 | 37.90 | I | C |
| ATOM | 3044 | CD1 | TYR | I | 194 | 54.373 | 30.781 | 2.874 | 1.00 | 34.82 | I | C |
| ATOM | 3045 | CE1 | TYR | I | 194 | 54.695 | 29.670 | 2.120 | 1.00 | 33.45 | I | C |
| ATOM | 3046 | CD2 | TYR | I | 194 | 56.679 | 31.118 | 3.399 | 1.00 | 33.78 | I | C |
| ATOM | 3047 | CE2 | TYR | I | 194 | 57.016 | 30.010 | 2.651 | 1.00 | 39.20 | I | C |
| ATOM | 3048 | CZ | TYR | I | 194 | 56.023 | 29.286 | 2.011 | 1.00 | 37.48 | I | C |
| ATOM | 3049 | OH | TYR | I | 194 | 56.369 | 28.179 | 1.268 | 1.00 | 43.40 | I | O |
| ATOM | 3050 | C | TYR | I | 194 | 54.215 | 35.051 | 4.474 | 1.00 | 40.70 | I | C |
| ATOM | 3051 | O | TYR | I | 194 | 55.063 | 35.591 | 5.178 | 1.00 | 43.16 | I | O |
| ATOM | 3052 | N | ILE | I | 195 | 52.943 | 35.401 | 4.484 | 1.00 | 39.44 | I | N |
| ATOM | 3053 | CA | ILE | I | 195 | 52.457 | 36.489 | 5.309 | 1.00 | 40.30 | I | C |
| ATOM | 3054 | CB | ILE | I | 195 | 51.981 | 37.654 | 4.411 | 1.00 | 37.70 | I | C |
| ATOM | 3055 | CG2 | ILE | I | 195 | 51.175 | 38.676 | 5.213 | 1.00 | 34.57 | I | C |
| ATOM | 3056 | CG1 | ILE | I | 195 | 53.176 | 38.285 | 3.701 | 1.00 | 40.36 | I | C |
| ATOM | 3057 | CD1 | ILE | I | 195 | 52.804 | 39.451 | 2.819 | 1.00 | 46.50 | I | C |
| ATOM | 3058 | C | ILE | I | 195 | 51.266 | 35.994 | 6.117 | 1.00 | 41.77 | I | C |
| ATOM | 3059 | O | ILE | I | 195 | 50.359 | 35.391 | 5.547 | 1.00 | 37.59 | I | O |
| ATOM | 3060 | N | CYS | I | 196 | 51.285 | 36.192 | 7.436 | 1.00 | 41.16 | I | N |
| ATOM | 3061 | CA | CYS | I | 196 | 50.141 | 35.781 | 8.252 | 1.00 | 42.86 | I | C |
| ATOM | 3062 | C | CYS | I | 196 | 49.297 | 37.027 | 8.487 | 1.00 | 46.05 | I | C |
| ATOM | 3063 | O | CYS | I | 196 | 49.806 | 38.101 | 8.833 | 1.00 | 43.18 | I | O |
| ATOM | 3064 | CB | CYS | I | 196 | 50.555 | 35.132 | 9.579 | 1.00 | 44.98 | I | C |
| ATOM | 3065 | SG | CYS | I | 196 | 51.127 | 36.279 | 10.865 | 1.00 | 41.69 | I | S |
| ATOM | 3066 | N | ASN | I | 197 | 48.003 | 36.888 | 8.245 | 1.00 | 45.29 | I | N |
| ATOM | 3067 | CA | ASN | I | 197 | 47.090 | 38.004 | 8.380 | 1.00 | 46.16 | I | C |
| ATOM | 3068 | CB | ASN | I | 197 | 46.181 | 38.072 | 7.149 | 1.00 | 49.52 | I | C |
| ATOM | 3069 | CG | ASN | I | 197 | 46.941 | 37.797 | 5.849 | 1.00 | 50.03 | I | C |
| ATOM | 3070 | OD1 | ASN | I | 197 | 46.796 | 36.729 | 5.249 | 1.00 | 50.91 | I | O |
| ATOM | 3071 | ND2 | ASN | I | 197 | 47.788 | 38.739 | 5.442 | 1.00 | 46.11 | I | N |
| ATOM | 3072 | C | ASN | I | 197 | 46.292 | 37.883 | 9.665 | 1.00 | 44.54 | I | C |
| ATOM | 3073 | O | ASN | I | 197 | 45.407 | 37.041 | 9.795 | 1.00 | 44.80 | I | O |
| ATOM | 3074 | N | VAL | I | 198 | 46.633 | 38.737 | 10.620 | 1.00 | 40.67 | I | N |
| ATOM | 3075 | CA | VAL | I | 198 | 45.980 | 38.729 | 11.907 | 1.00 | 35.07 | I | C |
| ATOM | 3076 | CB | VAL | I | 198 | 47.003 | 38.838 | 13.022 | 1.00 | 28.63 | I | C |
| ATOM | 3077 | CG1 | VAL | I | 198 | 46.313 | 38.928 | 14.358 | 1.00 | 25.94 | I | C |
| ATOM | 3078 | CG2 | VAL | I | 198 | 47.939 | 37.656 | 12.960 | 1.00 | 26.01 | I | C |
| ATOM | 3079 | C | VAL | I | 198 | 44.981 | 39.859 | 12.012 | 1.00 | 39.62 | I | C |
| ATOM | 3080 | O | VAL | I | 198 | 45.246 | 40.987 | 11.597 | 1.00 | 44.00 | I | O |
| ATOM | 3081 | N | ASN | I | 199 | 43.833 | 39.551 | 12.594 | 1.00 | 40.83 | I | N |
| ATOM | 3082 | CA | ASN | I | 199 | 42.796 | 40.536 | 12.740 | 1.00 | 40.85 | I | C |
| ATOM | 3083 | CB | ASN | I | 199 | 41.802 | 40.404 | 11.577 | 1.00 | 47.12 | I | C |
| ATOM | 3084 | CG | ASN | I | 199 | 40.879 | 41.600 | 11.457 | 1.00 | 52.96 | I | C |
| ATOM | 3085 | OD1 | ASN | I | 199 | 39.691 | 41.516 | 11.772 | 1.00 | 57.01 | I | O |
| ATOM | 3086 | ND2 | ASN | I | 199 | 41.420 | 42.722 | 10.995 | 1.00 | 55.23 | I | N |
| ATOM | 3087 | C | ASN | I | 199 | 42.107 | 40.367 | 14.093 | 1.00 | 42.27 | I | C |
| ATOM | 3088 | O | ASN | I | 199 | 41.688 | 39.270 | 14.465 | 1.00 | 39.23 | I | O |
| ATOM | 3089 | N | HIS | I | 200 | 42.063 | 41.464 | 14.844 | 1.00 | 38.64 | I | N |
| ATOM | 3090 | CA | HIS | I | 200 | 41.429 | 41.511 | 16.150 | 1.00 | 39.12 | I | C |
| ATOM | 3091 | CB | HIS | I | 200 | 42.480 | 41.783 | 17.239 | 1.00 | 36.39 | I | C |
| ATOM | 3092 | CG | HIS | I | 200 | 41.921 | 41.831 | 18.629 | 1.00 | 30.85 | I | C |
| ATOM | 3093 | CD2 | HIS | I | 200 | 41.332 | 40.877 | 19.388 | 1.00 | 30.89 | I | C |
| ATOM | 3094 | ND1 | HIS | I | 200 | 41.924 | 42.979 | 19.390 | 1.00 | 29.99 | I | N |
| ATOM | 3095 | CE1 | HIS | I | 200 | 41.358 | 42.730 | 20.559 | 1.00 | 33.89 | I | C |
| ATOM | 3096 | NE2 | HIS | I | 200 | 40.990 | 41.462 | 20.583 | 1.00 | 30.95 | I | N |
| ATOM | 3097 | C | HIS | I | 200 | 40.430 | 42.663 | 16.051 | 1.00 | 41.31 | I | C |
| ATOM | 3098 | O | HIS | I | 200 | 40.804 | 43.831 | 16.188 | 1.00 | 43.45 | I | O |
| ATOM | 3099 | N | LYS | I | 201 | 39.169 | 42.318 | 15.799 | 1.00 | 40.19 | I | N |
| ATOM | 3100 | CA | LYS | I | 201 | 38.091 | 43.292 | 15.635 | 1.00 | 44.89 | I | C |
| ATOM | 3101 | CB | LYS | I | 201 | 36.774 | 42.570 | 15.292 | 1.00 | 49.52 | I | C |
| ATOM | 3102 | CG | LYS | I | 201 | 36.848 | 41.603 | 14.101 | 1.00 | 55.52 | I | C |
| ATOM | 3103 | CD | LYS | I | 201 | 36.480 | 42.260 | 12.763 | 1.00 | 57.53 | I | C |
| ATOM | 3104 | CE | LYS | I | 201 | 34.969 | 42.236 | 12.506 | 1.00 | 58.54 | I | C |
| ATOM | 3105 | NZ | LYS | I | 201 | 34.197 | 43.069 | 13.477 | 1.00 | 55.66 | I | N |
| ATOM | 3106 | C | LYS | I | 201 | 37.843 | 44.290 | 16.784 | 1.00 | 44.13 | I | C |
| ATOM | 3107 | O | LYS | I | 201 | 37.722 | 45.490 | 16.536 | 1.00 | 45.39 | I | O |
| ATOM | 3108 | N | PRO | I | 202 | 37.817 | 43.819 | 18.049 | 1.00 | 39.39 | I | N |
| ATOM | 3109 | CD | PRO | I | 202 | 37.938 | 42.416 | 18.497 | 1.00 | 32.76 | I | C |
| ATOM | 3110 | CA | PRO | I | 202 | 37.570 | 44.716 | 19.189 | 1.00 | 37.03 | I | C |
| ATOM | 3111 | CB | PRO | I | 202 | 37.625 | 43.764 | 20.384 | 1.00 | 39.07 | I | C |
| ATOM | 3112 | CG | PRO | I | 202 | 37.183 | 42.426 | 19.787 | 1.00 | 33.85 | I | C |
| ATOM | 3113 | C | PRO | I | 202 | 38.504 | 45.916 | 19.365 | 1.00 | 41.64 | I | C |
| ATOM | 3114 | O | PRO | I | 202 | 38.143 | 46.906 | 20.008 | 1.00 | 39.82 | I | O |
| ATOM | 3115 | N | SER | I | 203 | 39.700 | 45.825 | 18.802 | 1.00 | 40.71 | I | N |
| ATOM | 3116 | CA | SER | I | 203 | 40.674 | 46.904 | 18.904 | 1.00 | 43.90 | I | C |
| ATOM | 3117 | CB | SER | I | 203 | 41.955 | 46.377 | 19.537 | 1.00 | 39.78 | I | C |
| ATOM | 3118 | OG | SER | I | 203 | 42.527 | 45.382 | 18.708 | 1.00 | 41.16 | I | O |
| ATOM | 3119 | C | SER | I | 203 | 40.998 | 47.451 | 17.517 | 1.00 | 47.31 | I | C |
| ATOM | 3120 | O | SER | I | 203 | 41.855 | 48.327 | 17.371 | 1.00 | 47.42 | I | O |
| ATOM | 3121 | N | ASN | I | 204 | 40.304 | 46.918 | 16.510 | 1.00 | 51.94 | I | N |
| ATOM | 3122 | CA | ASN | I | 204 | 40.491 | 47.286 | 15.105 | 1.00 | 55.01 | I | C |
| ATOM | 3123 | CB | ASN | I | 204 | 39.895 | 48.664 | 14.792 | 1.00 | 64.80 | I | C |
| ATOM | 3124 | CG | ASN | I | 204 | 38.367 | 48.640 | 14.742 | 1.00 | 71.09 | I | C |
| ATOM | 3125 | OD1 | ASN | I | 204 | 37.770 | 47.907 | 13.941 | 1.00 | 73.52 | I | O |
| ATOM | 3126 | ND2 | ASN | I | 204 | 37.728 | 49.430 | 15.608 | 1.00 | 72.64 | I | N |
| ATOM | 3127 | C | ASN | I | 204 | 41.956 | 47.201 | 14.728 | 1.00 | 51.99 | I | C |
| ATOM | 3128 | O | ASN | I | 204 | 42.543 | 48.141 | 14.192 | 1.00 | 53.61 | I | O |
| ATOM | 3129 | N | THR | I | 205 | 42.544 | 46.061 | 15.071 | 1.00 | 48.84 | I | N |
| ATOM | 3130 | CA | THR | I | 205 | 43.941 | 45.792 | 14.789 | 1.00 | 45.90 | I | C |
| ATOM | 3131 | CB | THR | I | 205 | 44.639 | 45.158 | 16.009 | 1.00 | 46.40 | I | C |
| ATOM | 3132 | OG1 | THR | I | 205 | 44.472 | 46.013 | 17.140 | 1.00 | 45.49 | I | O |
| ATOM | 3133 | CG2 | THR | I | 205 | 46.127 | 44.959 | 15.749 | 1.00 | 44.71 | I | C |
| ATOM | 3134 | C | THR | I | 205 | 44.030 | 44.828 | 13.623 | 1.00 | 45.00 | I | C |
| ATOM | 3135 | O | THR | I | 205 | 43.432 | 43.752 | 13.646 | 1.00 | 41.09 | I | O |
| ATOM | 3136 | N | LYS | I | 206 | 44.742 | 45.255 | 12.588 | 1.00 | 46.06 | I | N |
| ATOM | 3137 | CA | LYS | I | 206 | 44.959 | 44.440 | 11.407 | 1.00 | 46.32 | I | C |
| ATOM | 3138 | CB | LYS | I | 206 | 44.205 | 45.007 | 10.200 | 1.00 | 49.51 | I | C |
| ATOM | 3139 | CG | LYS | I | 206 | 44.155 | 44.049 | 9.008 | 1.00 | 57.53 | I | C |
| ATOM | 3140 | CD | LYS | I | 206 | 43.471 | 44.669 | 7.785 | 1.00 | 62.20 | I | C |
| ATOM | 3141 | CE | LYS | I | 206 | 43.406 | 43.685 | 6.601 | 1.00 | 63.86 | I | C |
| ATOM | 3142 | NZ | LYS | I | 206 | 42.378 | 42.601 | 6.771 | 1.00 | 64.13 | I | N |
| ATOM | 3143 | C | LYS | I | 206 | 46.466 | 44.477 | 11.169 | 1.00 | 42.98 | I | C |
| ATOM | 3144 | O | LYS | I | 206 | 47.045 | 45.542 | 10.974 | 1.00 | 41.83 | I | O |
| ATOM | 3145 | N | VAL | I | 207 | 47.102 | 43.316 | 11.272 | 1.00 | 40.67 | I | N |
| ATOM | 3146 | CA | VAL | I | 207 | 48.541 | 43.198 | 11.071 | 1.00 | 39.89 | I | C |
| ATOM | 3147 | CB | VAL | I | 207 | 49.261 | 42.771 | 12.369 | 1.00 | 40.71 | I | C |
| ATOM | 3148 | CG1 | VAL | I | 207 | 50.747 | 42.491 | 12.089 | 1.00 | 36.29 | I | C |
| ATOM | 3149 | CG2 | VAL | I | 207 | 49.091 | 43.828 | 13.450 | 1.00 | 36.88 | I | C |
| ATOM | 3150 | C | VAL | I | 207 | 48.866 | 42.154 | 10.014 | 1.00 | 40.20 | I | C |
| ATOM | 3151 | O | VAL | I | 207 | 48.254 | 41.092 | 9.970 | 1.00 | 44.65 | I | O |
| ATOM | 3152 | N | ASP | I | 208 | 49.835 | 42.471 | 9.164 | 1.00 | 45.25 | I | N |
| ATOM | 3153 | CA | ASP | I | 208 | 50.287 | 41.556 | 8.124 | 1.00 | 41.40 | I | C |
| ATOM | 3154 | CB | ASP | I | 208 | 50.002 | 42.127 | 6.738 | 1.00 | 44.76 | I | C |
| ATOM | 3155 | CG | ASP | I | 208 | 48.521 | 42.294 | 6.472 | 1.00 | 49.94 | I | C |
| ATOM | 3156 | OD1 | ASP | I | 208 | 47.769 | 41.304 | 6.607 | 1.00 | 51.71 | I | O |
| ATOM | 3157 | OD2 | ASP | I | 208 | 48.103 | 43.423 | 6.132 | 1.00 | 54.17 | I | O |
| ATOM | 3158 | C | ASP | I | 208 | 51.784 | 41.393 | 8.332 | 1.00 | 41.93 | I | C |
| ATOM | 3159 | O | ASP | I | 208 | 52.574 | 42.262 | 7.967 | 1.00 | 43.79 | I | O |
| ATOM | 3160 | N | LYS | I | 209 | 52.172 | 40.294 | 8.959 | 1.00 | 36.52 | I | N |
| ATOM | 3161 | CA | LYS | I | 209 | 53.571 | 40.062 | 9.228 | 1.00 | 33.25 | I | C |
| ATOM | 3162 | CB | LYS | I | 209 | 53.746 | 39.484 | 10.632 | 1.00 | 35.27 | I | C |
| ATOM | 3163 | CG | LYS | I | 209 | 55.158 | 39.604 | 11.180 | 1.00 | 32.74 | I | C |
| ATOM | 3164 | CD | LYS | I | 209 | 55.504 | 41.056 | 11.412 | 1.00 | 41.32 | I | C |
| ATOM | 3165 | CE | LYS | I | 209 | 56.932 | 41.218 | 11.881 | 1.00 | 46.97 | I | C |
| ATOM | 3166 | NZ | LYS | I | 209 | 57.222 | 42.653 | 12.123 | 1.00 | 50.65 | I | N |
| ATOM | 3167 | C | LYS | I | 209 | 54.193 | 39.122 | 8.219 | 1.00 | 35.06 | I | C |
| ATOM | 3168 | O | LYS | I | 209 | 53.662 | 38.050 | 7.964 | 1.00 | 32.21 | I | O |
| ATOM | 3169 | N | LYS | I | 210 | 55.307 | 39.548 | 7.627 | 1.00 | 40.53 | I | N |
| ATOM | 3170 | CA | LYS | I | 210 | 56.033 | 38.721 | 6.675 | 1.00 | 42.76 | I | C |
| ATOM | 3171 | CB | LYS | I | 210 | 56.827 | 39.589 | 5.684 | 1.00 | 49.39 | I | C |
| ATOM | 3172 | CG | LYS | I | 210 | 57.509 | 38.802 | 4.543 | 1.00 | 52.83 | I | C |
| ATOM | 3173 | CD | LYS | I | 210 | 58.458 | 39.680 | 3.720 | 1.00 | 56.37 | I | C |
| ATOM | 3174 | CE | LYS | I | 210 | 59.319 | 38.840 | 2.772 | 1.00 | 58.93 | I | C |
| ATOM | 3175 | NZ | LYS | I | 210 | 60.546 | 39.571 | 2.319 | 1.00 | 57.04 | I | N |
| ATOM | 3176 | C | LYS | I | 210 | 56.985 | 37.890 | 7.542 | 1.00 | 46.11 | I | C |
| ATOM | 3177 | O | LYS | I | 210 | 57.625 | 38.430 | 8.448 | 1.00 | 44.13 | I | O |
| ATOM | 3178 | N | VAL | I | 211 | 57.034 | 36.578 | 7.303 | 1.00 | 43.26 | I | N |
| ATOM | 3179 | CA | VAL | I | 211 | 57.901 | 35.686 | 8.074 | 1.00 | 43.66 | I | C |
| ATOM | 3180 | CB | VAL | I | 211 | 57.104 | 34.506 | 8.674 | 1.00 | 37.81 | I | C |
| ATOM | 3181 | CG1 | VAL | I | 211 | 58.012 | 33.590 | 9.456 | 1.00 | 39.16 | I | C |
| ATOM | 3182 | CG2 | VAL | I | 211 | 56.015 | 35.031 | 9.565 | 1.00 | 36.46 | I | C |
| ATOM | 3183 | C | VAL | I | 211 | 59.013 | 35.147 | 7.189 | 1.00 | 46.55 | I | C |
| ATOM | 3184 | O | VAL | I | 211 | 58.746 | 34.533 | 6.155 | 1.00 | 52.58 | I | O |
| ATOM | 3185 | N | GLU | I | 212 | 60.254 | 35.351 | 7.618 | 1.00 | 49.32 | I | N |
| ATOM | 3186 | CA | GLU | I | 212 | 61.423 | 34.903 | 6.859 | 1.00 | 56.31 | I | C |
| ATOM | 3187 | CB | GLU | I | 212 | 61.856 | 36.010 | 5.887 | 1.00 | 60.49 | I | C |
| ATOM | 3188 | CG | GLU | I | 212 | 62.338 | 37.295 | 6.566 | 1.00 | 63.88 | I | C |
| ATOM | 3189 | CD | GLU | I | 212 | 62.356 | 38.495 | 5.632 | 1.00 | 66.40 | I | C |
| ATOM | 3190 | OE1 | GLU | I | 212 | 62.799 | 38.370 | 4.465 | 1.00 | 69.31 | 1 | O |
| ATOM | 3191 | OE2 | GLU | I | 212 | 61.916 | 39.573 | 6.074 | 1.00 | 68.93 | I | O |
| ATOM | 3192 | C | GLU | I | 212 | 62.589 | 34.535 | 7.786 | 1.00 | 56.78 | I | C |
| ATOM | 3193 | O | GLU | I | 212 | 62.650 | 35.000 | 8.926 | 1.00 | 61.68 | I | O |
| ATOM | 3194 | N | PRO | I | 213 | 63.544 | 33.718 | 7.297 | 1.00 | 57.35 | I | N |
| ATOM | 3195 | CD | PRO | I | 213 | 63.557 | 33.100 | 5.954 | 1.00 | 58.27 | I | C |
| ATOM | 3196 | CA | PRO | I | 213 | 64.712 | 33.287 | 8.083 | 1.00 | 56.09 | I | C |
| ATOM | 3197 | CB | PRO | I | 213 | 65.340 | 32.214 | 7.190 | 1.00 | 56.04 | I | C |
| ATOM | 3198 | CG | PRO | I | 213 | 64.997 | 32.687 | 5.796 | 1.00 | 54.69 | I | C |
| ATOM | 3199 | C | PRO | I | 213 | 65.707 | 34.405 | 8.413 | 1.00 | 55.59 | I | C |
| ATOM | 3200 | O | PRO | I | 213 | 66.235 | 34.475 | 9.525 | 1.00 | 54.53 | I | O |
| ATOM | 3201 | N | ASP | L | 1 | 9.768 | 8.303 | −22.483 | 1.00 | 46.07 | L | N |
| ATOM | 3202 | CA | ASP | L | 1 | 10.245 | 8.465 | −21.116 | 1.00 | 48.93 | L | C |
| ATOM | 3203 | CB | ASP | L | 1 | 9.138 | 8.069 | −20.116 | 1.00 | 49.04 | L | C |
| ATOM | 3204 | CG | ASP | L | 1 | 7.878 | 8.928 | −20.240 | 1.00 | 54.02 | L | C |
| ATOM | 3205 | OD1 | ASP | L | 1 | 7.825 | 9.836 | −21.108 | 1.00 | 53.04 | L | O |
| ATOM | 3206 | OD2 | ASP | L | 1 | 6.930 | 8.689 | −19.456 | 1.00 | 57.25 | L | O |
| ATOM | 3207 | C | ASP | L | 1 | 11.466 | 7.589 | −20.848 | 1.00 | 48.39 | L | C |
| ATOM | 3208 | O | ASP | L | 1 | 11.663 | 6.573 | −21.511 | 1.00 | 52.62 | L | O |
| ATOM | 3209 | N | ILE | L | 2 | 12.272 | 7.975 | −19.858 | 1.00 | 46.73 | L | N |
| ATOM | 3210 | CA | ILE | L | 2 | 13.447 | 7.195 | −19.480 | 1.00 | 38.20 | L | C |
| ATOM | 3211 | CB | ILE | L | 2 | 14.546 | 8.076 | −18.841 | 1.00 | 38.47 | L | C |
| ATOM | 3212 | CG2 | ILE | L | 2 | 15.597 | 7.222 | −18.148 | 1.00 | 32.51 | L | C |
| ATOM | 3213 | CG1 | ILE | L | 2 | 15.209 | 8.929 | −19.913 | 1.00 | 37.14 | L | C |
| ATOM | 3214 | CD1 | ILE | L | 2 | 16.312 | 9.816 | −19.386 | 1.00 | 39.54 | L | C |
| ATOM | 3215 | C | ILE | L | 2 | 12.996 | 6.141 | −18.475 | 1.00 | 39.66 | L | C |
| ATOM | 3216 | O | ILE | L | 2 | 12.321 | 6.456 | −17.490 | 1.00 | 31.25 | L | O |
| ATOM | 3217 | N | GLN | L | 3 | 13.395 | 4.895 | −18.721 | 1.00 | 38.45 | L | N |
| ATOM | 3218 | CA | GLN | L | 3 | 13.029 | 3.775 | −17.854 | 1.00 | 37.17 | L | C |
| ATOM | 3219 | CB | GLN | L | 3 | 12.815 | 2.495 | −18.697 | 1.00 | 41.44 | L | C |
| ATOM | 3220 | CG | GLN | L | 3 | 12.011 | 2.676 | −20.016 | 1.00 | 41.87 | L | C |
| ATOM | 3221 | CD | GLN | L | 3 | 10.644 | 3.311 | −19.797 | 1.00 | 46.39 | L | C |
| ATOM | 3222 | OE1 | GLN | L | 3 | 9.904 | 2.922 | −18.890 | 1.00 | 47.96 | L | O |
| ATOM | 3223 | NE2 | GLN | L | 3 | 10.315 | 4.311 | −20.613 | 1.00 | 45.32 | L | N |
| ATOM | 3224 | C | GLN | L | 3 | 14.074 | 3.505 | −16.762 | 1.00 | 33.67 | L | C |
| ATOM | 3225 | O | GLN | L | 3 | 15.224 | 3.207 | −17.056 | 1.00 | 35.41 | L | O |
| ATOM | 3226 | N | MET | L | 4 | 13.668 | 3.613 | −15.502 | 1.00 | 32.94 | L | N |
| ATOM | 3227 | CA | MET | L | 4 | 14.572 | 3.356 | −14.374 | 1.00 | 28.69 | L | C |
| ATOM | 3228 | CB | MET | L | 4 | 14.369 | 4.370 | −13.223 | 1.00 | 27.15 | L | C |
| ATOM | 3229 | CG | MET | L | 4 | 14.684 | 5.829 | −13.517 | 1.00 | 20.73 | L | C |
| ATOM | 3230 | SD | MET | L | 4 | 16.305 | 6.090 | −14.310 | 1.00 | 31.65 | L | S |
| ATOM | 3231 | CE | MET | L | 4 | 17.447 | 5.409 | −13.162 | 1.00 | 14.15 | L | C |
| ATOM | 3232 | C | MET | L | 4 | 14.252 | 1.991 | −13.791 | 1.00 | 27.91 | L | C |
| ATOM | 3233 | O | MET | L | 4 | 13.169 | 1.807 | −13.235 | 1.00 | 24.47 | L | O |
| ATOM | 3234 | N | THR | L | 5 | 15.185 | 1.050 | −13.861 | 1.00 | 18.19 | L | N |
| ATOM | 3235 | CA | THR | L | 5 | 14.918 | −0.249 | −13.276 | 1.00 | 21.22 | L | C |
| ATOM | 3236 | CB | THR | L | 5 | 15.265 | −1.371 | −14.220 | 1.00 | 20.55 | L | C |
| ATOM | 3237 | OG1 | THR | L | 5 | 14.716 | −1.075 | −15.497 | 1.00 | 32.15 | L | O |
| ATOM | 3238 | CG2 | THR | L | 5 | 14.692 | −2.700 | −13.691 | 1.00 | 18.76 | L | C |
| ATOM | 3239 | C | THR | L | 5 | 15.744 | −0.464 | −12.011 | 1.00 | 22.93 | L | C |
| ATOM | 3240 | O | THR | L | 5 | 16.967 | −0.537 | −12.090 | 1.00 | 19.59 | L | O |
| ATOM | 3241 | N | GLN | L | 6 | 15.082 | −0.615 | −10.861 | 1.00 | 19.21 | L | N |
| ATOM | 3242 | CA | GLN | L | 6 | 15.794 | −0.843 | −9.598 | 1.00 | 17.71 | L | C |
| ATOM | 3243 | CB | GLN | L | 6 | 15.093 | −0.192 | −8.399 | 1.00 | 13.44 | L | C |
| ATOM | 3244 | CG | GLN | L | 6 | 15.163 | 1.320 | −8.334 | 1.00 | 24.27 | L | C |
| ATOM | 3245 | CD | GLN | L | 6 | 14.564 | 1.893 | −7.043 | 1.00 | 19.84 | L | C |
| ATOM | 3246 | OE1 | GLN | L | 6 | 13.767 | 2.822 | −7.085 | 1.00 | 23.61 | L | O |
| ATOM | 3247 | NE2 | GLN | L | 6 | 14.974 | 1.350 | −5.895 | 1.00 | 23.05 | L | N |
| ATOM | 3248 | C | GLN | L | 6 | 15.884 | −2.321 | −9.327 | 1.00 | 19.02 | L | C |
| ATOM | 3249 | O | GLN | L | 6 | 14.929 | −3.058 | −9.571 | 1.00 | 27.81 | L | O |
| ATOM | 3250 | N | SER | L | 7 | 16.986 | −2.727 | −8.712 | 1.00 | 17.99 | L | N |
| ATOM | 3251 | CA | SER | L | 7 | 17.231 | −4.126 | −8.376 | 1.00 | 23.94 | L | C |
| ATOM | 3252 | CB | SER | L | 7 | 18.172 | −4.773 | −9.402 | 1.00 | 19.90 | L | C |
| ATOM | 3253 | OG | SER | L | 7 | 17.521 | −4.983 | −10.641 | 1.00 | 39.99 | L | O |
| ATOM | 3254 | C | SER | L | 7 | 17.882 | −4.235 | −7.004 | 1.00 | 24.03 | L | C |
| ATOM | 3255 | O | SER | L | 7 | 18.805 | −3.475 | −6.693 | 1.00 | 28.71 | L | O |
| ATOM | 3256 | N | PRO | L | 8 | 17.382 | −5.146 | −6.145 | 1.00 | 23.71 | L | N |
| ATOM | 3257 | CD | PRO | L | 8 | 18.054 | −5.468 | −4.868 | 1.00 | 23.36 | L | C |
| ATOM | 3258 | CA | PRO | L | 8 | 16.257 | −6.065 | −6.379 | 1.00 | 22.52 | L | C |
| ATOM | 3259 | CB | PRO | L | 8 | 16.568 | −7.216 | −5.438 | 1.00 | 20.94 | L | C |
| ATOM | 3260 | CG | PRO | L | 8 | 17.122 | −6.490 | −4.250 | 1.00 | 22.33 | L | C |
| ATOM | 3261 | C | PRO | L | 8 | 14.970 | −5.382 | −5.946 | 1.00 | 23.30 | L | C |
| ATOM | 3262 | O | PRO | L | 8 | 15.021 | −4.272 | −5.421 | 1.00 | 23.61 | L | O |
| ATOM | 3263 | N | SER | L | 9 | 13.822 | −6.010 | −6.190 | 1.00 | 22.26 | L | N |
| ATOM | 3264 | CA | SER | L | 9 | 12.552 | −5.413 | −5.762 | 1.00 | 22.78 | L | C |
| ATOM | 3265 | CB | SER | L | 9 | 11.351 | −6.115 | −6.397 | 1.00 | 26.63 | L | C |
| ATOM | 3266 | OG | SER | L | 9 | 11.011 | −5.483 | −7.623 | 1.00 | 40.11 | L | O |
| ATOM | 3267 | C | SER | L | 9 | 12.460 | −5.448 | −4.242 | 1.00 | 15.31 | L | C |
| ATOM | 3268 | O | SER | L | 9 | 11.905 | −4.555 | −3.630 | 1.00 | 17.61 | L | O |
| ATOM | 3269 | N | SER | L | 10 | 13.028 | −6.473 | −3.627 | 1.00 | 18.85 | L | N |
| ATOM | 3270 | CA | SER | L | 10 | 13.030 | −6.546 | −2.178 | 1.00 | 25.27 | L | C |
| ATOM | 3271 | CB | SER | L | 10 | 11.776 | −7.265 | −1.669 | 1.00 | 29.58 | L | C |
| ATOM | 3272 | OG | SER | L | 10 | 11.957 | −8.666 | −1.676 | 1.00 | 36.72 | L | O |
| ATOM | 3273 | C | SER | L | 10 | 14.288 | −7.274 | −1.728 | 1.00 | 18.32 | L | C |
| ATOM | 3274 | O | SER | L | 10 | 14.783 | −8.142 | −2.435 | 1.00 | 22.33 | L | O |
| ATOM | 3275 | N | LEU | L | 11 | 14.850 | −6.877 | −0.591 | 1.00 | 23.31 | L | N |
| ATOM | 3276 | CA | LEU | L | 11 | 16.041 | −7.555 | −0.085 | 1.00 | 22.93 | L | C |
| ATOM | 3277 | CB | LEU | L | 11 | 17.322 | −6.816 | −0.494 | 1.00 | 27.54 | L | C |
| ATOM | 3278 | CG | LEU | L | 11 | 17.853 | −5.516 | 0.097 | 1.00 | 28.82 | L | C |
| ATOM | 3279 | CD1 | LEU | L | 11 | 18.806 | −5.829 | 1.249 | 1.00 | 31.55 | L | C |
| ATOM | 3280 | CD2 | LEU | L | 11 | 18.635 | −4.793 | −0.983 | 1.00 | 32.79 | L | C |
| ATOM | 3281 | C | LEU | L | 11 | 15.979 | −7.808 | 1.410 | 1.00 | 23.43 | L | C |
| ATOM | 3282 | O | LEU | L | 11 | 15.460 | −7.001 | 2.174 | 1.00 | 30.94 | L | O |
| ATOM | 3283 | N | SER | L | 12 | 16.428 | −8.991 | 1.800 | 1.00 | 30.87 | L | N |
| ATOM | 3284 | CA | SER | L | 12 | 16.428 | −9.406 | 3.196 | 1.00 | 30.51 | L | C |
| ATOM | 3285 | CB | SER | L | 12 | 16.103 | −10.897 | 3.283 | 1.00 | 31.26 | L | C |
| ATOM | 3286 | OG | SER | L | 12 | 14.985 | −11.205 | 2.460 | 1.00 | 39.85 | L | O |
| ATOM | 3287 | C | SER | L | 12 | 17.787 | −9.134 | 3.820 | 1.00 | 29.10 | L | C |
| ATOM | 3288 | O | SER | L | 12 | 18.823 | −9.601 | 3.335 | 1.00 | 24.10 | L | O |
| ATOM | 3289 | N | ALA | L | 13 | 17.787 | −8.381 | 4.904 | 1.00 | 20.94 | L | N |
| ATOM | 3290 | CA | ALA | L | 13 | 19.039 | −8.089 | 5.547 | 1.00 | 24.58 | L | C |
| ATOM | 3291 | CB | ALA | L | 13 | 19.573 | −6.752 | 5.045 | 1.00 | 23.72 | L | C |
| ATOM | 3292 | C | ALA | L | 13 | 18.928 | −8.103 | 7.060 | 1.00 | 23.99 | L | C |
| ATOM | 3293 | O | ALA | L | 13 | 17.834 | −8.140 | 7.621 | 1.00 | 25.69 | L | O |
| ATOM | 3294 | N | SER | L | 14 | 20.084 | −8.122 | 7.704 | 1.00 | 25.12 | L | N |
| ATOM | 3295 | CA | SER | L | 14 | 20.181 | −8.124 | 9.154 | 1.00 | 26.42 | L | C |
| ATOM | 3296 | CB | SER | L | 14 | 20.966 | −9.353 | 9.611 | 1.00 | 21.14 | L | C |
| ATOM | 3297 | OG | SER | L | 14 | 20.314 | −10.532 | 9.174 | 1.00 | 29.70 | L | O |
| ATOM | 3298 | C | SER | L | 14 | 20.879 | −6.839 | 9.612 | 1.00 | 24.73 | L | C |
| ATOM | 3299 | O | SER | L | 14 | 21.575 | −6.172 | 8.826 | 1.00 | 26.80 | L | O |
| ATOM | 3300 | N | VAL | L | 15 | 20.652 | −6.462 | 10.862 | 1.00 | 19.56 | L | N |
| ATOM | 3301 | CA | VAL | L | 15 | 21.280 | −5.266 | 11.395 | 1.00 | 25.05 | L | C |
| ATOM | 3302 | CB | VAL | L | 15 | 20.823 | −5.021 | 12.829 | 1.00 | 27.32 | L | C |
| ATOM | 3303 | CG1 | VAL | L | 15 | 21.605 | −3.859 | 13.460 | 1.00 | 24.72 | L | C |
| ATOM | 3304 | CG2 | VAL | L | 15 | 19.343 | −4.740 | 12.819 | 1.00 | 24.69 | L | C |
| ATOM | 3305 | C | VAL | L | 15 | 22.794 | −5.471 | 11.325 | 1.00 | 23.80 | L | C |
| ATOM | 3306 | O | VAL | L | 15 | 23.307 | −6.558 | 11.662 | 1.00 | 17.01 | L | O |
| ATOM | 3307 | N | GLY | L | 16 | 23.491 | −4.451 | 10.834 | 1.00 | 18.19 | L | N |
| ATOM | 3308 | CA | GLY | L | 16 | 24.937 | −4.548 | 10.695 | 1.00 | 26.08 | L | C |
| ATOM | 3309 | C | GLY | L | 16 | 25.430 | −4.865 | 9.286 | 1.00 | 28.12 | L | C |
| ATOM | 3310 | O | GLY | L | 16 | 26.587 | −4.605 | 8.968 | 1.00 | 26.82 | L | O |
| ATOM | 3311 | N | ASP | L | 17 | 24.561 | −5.400 | 8.432 | 1.00 | 27.87 | L | N |
| ATOM | 3312 | CA | ASP | L | 17 | 24.950 | −5.753 | 7.070 | 1.00 | 26.89 | L | C |
| ATOM | 3313 | CB | ASP | L | 17 | 23.834 | −6.550 | 6.383 | 1.00 | 28.27 | L | C |
| ATOM | 3314 | CG | ASP | L | 17 | 23.735 | −7.972 | 6.867 | 1.00 | 31.27 | L | C |
| ATOM | 3315 | OD1 | ASP | L | 17 | 24.666 | −8.468 | 7.532 | 1.00 | 38.37 | L | O |
| ATOM | 3316 | OD2 | ASP | L | 17 | 22.708 | −8.605 | 6.563 | 1.00 | 37.74 | L | O |
| ATOM | 3317 | C | ASP | L | 17 | 25.280 | −4.569 | 6.171 | 1.00 | 27.61 | L | C |
| ATOM | 3318 | O | ASP | L | 17 | 24.870 | −3.430 | 6.432 | 1.00 | 25.77 | L | O |
| ATOM | 3319 | N | ARG | L | 18 | 26.036 | −4.856 | 5.109 | 1.00 | 26.21 | L | N |
| ATOM | 3320 | CA | ARG | L | 18 | 26.361 | −3.848 | 4.105 | 1.00 | 31.32 | L | C |
| ATOM | 3321 | CB | ARG | L | 18 | 27.794 | −3.983 | 3.574 | 1.00 | 31.71 | L | C |
| ATOM | 3322 | CG | ARG | L | 18 | 28.207 | −2.840 | 2.616 | 1.00 | 35.58 | L | C |
| ATOM | 3323 | CD | ARG | L | 18 | 29.692 | −2.893 | 2.229 | 1.00 | 40.10 | L | C |
| ATOM | 3324 | NE | ARG | L | 18 | 30.179 | −1.607 | 1.717 | 1.00 | 42.03 | L | N |
| ATOM | 3325 | CZ | ARG | L | 18 | 30.298 | −1.300 | 0.422 | 1.00 | 45.29 | L | C |
| ATOM | 3326 | NH1 | ARG | L | 18 | 29.961 | −2.186 | −0.515 | 1.00 | 42.79 | L | N |
| ATOM | 3327 | NH2 | ARG | L | 18 | 30.763 | −0.106 | 0.061 | 1.00 | 33.71 | L | N |
| ATOM | 3328 | C | ARG | L | 18 | 25.354 | −4.143 | 2.996 | 1.00 | 28.39 | L | C |
| ATOM | 3329 | O | ARG | L | 18 | 25.336 | −5.236 | 2.427 | 1.00 | 29.72 | L | O |
| ATOM | 3330 | N | VAL | L | 19 | 24.491 | −3.179 | 2.717 | 1.00 | 23.69 | L | N |
| ATOM | 3331 | CA | VAL | L | 19 | 23.466 | −3.377 | 1.718 | 1.00 | 23.31 | L | C |
| ATOM | 3332 | CB | VAL | L | 19 | 22.089 | −3.204 | 2.375 | 1.00 | 17.56 | L | C |
| ATOM | 3333 | CG1 | VAL | L | 19 | 21.036 | −2.730 | 1.384 | 1.00 | 17.33 | L | C |
| ATOM | 3334 | CG2 | VAL | L | 19 | 21.695 | −4.522 | 2.969 | 1.00 | 22.53 | L | C |
| ATOM | 3335 | C | VAL | L | 19 | 23.633 | −2.506 | 0.499 | 1.00 | 20.16 | L | C |
| ATOM | 3336 | O | VAL | L | 19 | 23.979 | −1.336 | 0.612 | 1.00 | 19.83 | L | O |
| ATOM | 3337 | N | THR | L | 20 | 23.412 | −3.109 | −0.669 | 1.00 | 23.52 | L | N |
| ATOM | 3338 | CA | THR | L | 20 | 23.521 | −2.400 | −1.940 | 1.00 | 23.69 | L | C |
| ATOM | 3339 | CB | THR | L | 20 | 24.794 | −2.809 | −2.733 | 1.00 | 23.81 | L | C |
| ATOM | 3340 | OG1 | THR | L | 20 | 24.770 | −4.215 | −2.991 | 1.00 | 38.41 | L | O |
| ATOM | 3341 | CG2 | THR | L | 20 | 26.040 | −2.485 | −1.937 | 1.00 | 34.00 | L | C |
| ATOM | 3342 | C | THR | L | 20 | 22.282 | −2.587 | −2.808 | 1.00 | 21.99 | L | C |
| ATOM | 3343 | O | THR | L | 20 | 21.811 | −3.708 | −3.031 | 1.00 | 26.36 | L | O |
| ATOM | 3344 | N | ILE | L | 21 | 21.801 | −1.464 | −3.324 | 1.00 | 18.51 | L | N |
| ATOM | 3345 | CA | ILE | L | 21 | 20.627 | −1.398 | −4.170 | 1.00 | 21.14 | L | C |
| ATOM | 3346 | CB | ILE | L | 21 | 19.552 | −0.527 | −3.479 | 1.00 | 20.28 | L | C |
| ATOM | 3347 | CG2 | ILE | L | 21 | 18.310 | −0.429 | −4.327 | 1.00 | 25.11 | L | C |
| ATOM | 3348 | CG1 | ILE | L | 21 | 19.240 | −1.093 | −2.096 | 1.00 | 23.81 | L | C |
| ATOM | 3349 | CD1 | ILE | L | 21 | 18.302 | −0.238 | −1.285 | 1.00 | 27.89 | L | C |
| ATOM | 3350 | C | ILE | L | 21 | 21.062 | −0.687 | −5.450 | 1.00 | 24.71 | L | C |
| ATOM | 3351 | O | ILE | L | 21 | 21.729 | 0.356 | −5.377 | 1.00 | 19.24 | L | O |
| ATOM | 3352 | N | THR | L | 22 | 20.710 | −1.239 | −6.615 | 1.00 | 24.02 | L | N |
| ATOM | 3353 | CA | THR | L | 22 | 21.090 | −0.587 | −7.869 | 1.00 | 25.16 | L | C |
| ATOM | 3354 | CB | THR | L | 22 | 21.961 | −1.484 | −8.773 | 1.00 | 24.27 | L | C |
| ATOM | 3355 | OG1 | THR | L | 22 | 21.128 | −2.184 | −9.698 | 1.00 | 28.32 | L | O |
| ATOM | 3356 | CG2 | THR | L | 22 | 22.760 | −2.484 | −7.947 | 1.00 | 20.27 | L | C |
| ATOM | 3357 | C | THR | L | 22 | 19.918 | −0.039 | −8.677 | 1.00 | 27.17 | L | C |
| ATOM | 3358 | O | THR | L | 22 | 18.780 | −0.493 | −8.559 | 1.00 | 29.04 | L | O |
| ATOM | 3359 | N | CYS | L | 23 | 20.227 | 0.934 | −9.520 | 1.00 | 26.12 | L | N |
| ATOM | 3360 | CA | CYS | L | 23 | 19.248 | 1.591 | −10.376 | 1.00 | 30.96 | L | C |
| ATOM | 3361 | C | CYS | L | 23 | 19.931 | 1.678 | −11.746 | 1.00 | 28.06 | L | C |
| ATOM | 3362 | O | CYS | L | 23 | 21.097 | 2.043 | −11.839 | 1.00 | 32.78 | L | O |
| ATOM | 3363 | CB | CYS | L | 23 | 18.927 | 2.984 | −9.797 | 1.00 | 31.24 | L | C |
| ATOM | 3364 | SG | CYS | L | 23 | 17.679 | 4.018 | −10.638 | 1.00 | 52.72 | L | S |
| ATOM | 3365 | N | ARG | L | 24 | 19.207 | 1.296 | −12.789 | 0.50 | 28.54 | L | N |
| ATOM | 3366 | CA | ARG | L | 24 | 19.715 | 1.301 | −14.154 | 0.50 | 30.13 | L | C |
| ATOM | 3367 | CB | ARG | L | 24 | 19.790 | −0.143 | −14.656 | 0.50 | 33.37 | L | C |
| ATOM | 3368 | CG | ARG | L | 24 | 20.319 | −0.355 | −16.079 | 0.50 | 36.91 | L | C |
| ATOM | 3369 | CD | ARG | L | 24 | 19.206 | −0.393 | −17.148 | 0.50 | 41.03 | L | C |
| ATOM | 3370 | NE | ARG | L | 24 | 18.043 | −1.215 | −16.794 | 0.50 | 41.43 | L | N |
| ATOM | 3371 | CZ | ARG | L | 24 | 18.096 | −2.449 | −16.296 | 0.50 | 44.68 | L | C |
| ATOM | 3372 | NH1 | ARG | L | 24 | 16.976 | −3.099 | −16.015 | 0.50 | 48.05 | L | N |
| ATOM | 3373 | NH2 | ARG | L | 24 | 19.259 | −3.034 | −16.060 | 0.50 | 47.31 | L | N |
| ATOM | 3374 | C | ARG | L | 24 | 18.802 | 2.130 | −15.050 | 0.50 | 30.78 | L | C |
| ATOM | 3375 | O | ARG | L | 24 | 17.609 | 1.845 | −15.161 | 0.50 | 36.31 | L | O |
| ATOM | 3376 | N | ALA | L | 25 | 19.359 | 3.173 | −15.656 | 1.00 | 29.56 | L | N |
| ATOM | 3377 | CA | ALA | L | 25 | 18.599 | 4.038 | −16.546 | 1.00 | 26.53 | L | C |
| ATOM | 3378 | CB | ALA | L | 25 | 19.152 | 5.445 | −16.523 | 1.00 | 28.72 | L | C |
| ATOM | 3379 | C | ALA | L | 25 | 18.750 | 3.441 | −17.925 | 1.00 | 26.90 | L | C |
| ATOM | 3380 | O | ALA | L | 25 | 19.741 | 2.756 | −18.191 | 1.00 | 25.89 | L | O |
| ATOM | 3381 | N | SER | L | 26 | 17.759 | 3.673 | −18.783 | 1.00 | 30.22 | L | N |
| ATOM | 3382 | CA | SER | L | 26 | 17.784 | 3.144 | −20.144 | 1.00 | 33.15 | L | C |
| ATOM | 3383 | CB | SER | L | 26 | 16.404 | 3.262 | −20.810 | 1.00 | 33.46 | L | C |
| ATOM | 3384 | OG | SER | L | 26 | 15.800 | 4.532 | −20.608 | 1.00 | 34.09 | L | O |
| ATOM | 3385 | C | SER | L | 26 | 18.860 | 3.822 | −20.973 | 1.00 | 34.94 | L | C |
| ATOM | 3386 | O | SER | L | 26 | 19.427 | 3.213 | −21.878 | 1.00 | 46.23 | L | O |
| ATOM | 3387 | N | ARG | L | 27 | 19.178 | 5.062 | −20.609 | 1.00 | 37.10 | L | N |
| ATOM | 3388 | CA | ARG | L | 27 | 20.199 | 5.854 | −21.292 | 1.00 | 35.37 | L | C |
| ATOM | 3389 | CB | ARG | L | 27 | 19.556 | 6.728 | −22.387 | 1.00 | 29.83 | L | C |
| ATOM | 3390 | CG | ARG | L | 27 | 18.755 | 7.922 | −21.882 | 1.00 | 28.53 | L | C |
| ATOM | 3391 | CD | ARG | L | 27 | 18.373 | 8.812 | −23.039 | 1.00 | 32.96 | L | C |
| ATOM | 3392 | NE | ARG | L | 27 | 17.841 | 10.138 | −22.706 | 1.00 | 32.25 | L | N |
| ATOM | 3393 | CZ | ARG | L | 27 | 18.470 | 11.056 | −21.979 | 1.00 | 31.83 | L | C |
| ATOM | 3394 | NH1 | ARG | L | 27 | 19.656 | 10.798 | −21.471 | 1.00 | 30.46 | L | N |
| ATOM | 3395 | NH2 | ARG | L | 27 | 17.958 | 12.277 | −21.845 | 1.00 | 36.19 | L | N |
| ATOM | 3396 | C | ARG | L | 27 | 20.947 | 6.730 | −20.269 | 1.00 | 36.19 | L | C |
| ATOM | 3397 | O | ARG | L | 27 | 20.506 | 6.880 | −19.130 | 1.00 | 36.91 | L | O |
| ATOM | 3398 | N | ASP | L | 28 | 22.067 | 7.310 | −20.694 | 1.00 | 35.54 | L | N |
| ATOM | 3399 | CA | ASP | L | 28 | 22.903 | 8.157 | −19.852 | 1.00 | 32.95 | L | C |
| ATOM | 3400 | CB | ASP | L | 28 | 24.073 | 8.684 | −20.702 | 1.00 | 34.74 | L | C |
| ATOM | 3401 | CG | ASP | L | 28 | 25.124 | 9.457 | −19.896 | 1.00 | 36.72 | L | C |
| ATOM | 3402 | OD1 | ASP | L | 28 | 24.795 | 10.135 | −18.903 | 1.00 | 30.58 | L | O |
| ATOM | 3403 | OD2 | ASP | L | 28 | 26.303 | 9.421 | −20.306 | 1.00 | 40.89 | L | O |
| ATOM | 3404 | C | ASP | L | 28 | 22.101 | 9.313 | −19.233 | 1.00 | 31.53 | L | C |
| ATOM | 3405 | O | ASP | L | 28 | 21.484 | 10.093 | −19.947 | 1.00 | 23.71 | L | O |
| ATOM | 3406 | N | ILE | L | 29 | 22.105 | 9.417 | −17.903 | 1.00 | 23.93 | L | N |
| ATOM | 3407 | CA | ILE | L | 29 | 21.389 | 10.513 | −17.258 | 1.00 | 26.73 | L | C |
| ATOM | 3408 | CB | ILE | L | 29 | 20.291 | 10.032 | −16.271 | 1.00 | 26.31 | L | C |
| ATOM | 3409 | CG2 | ILE | L | 29 | 19.088 | 9.514 | −17.031 | 1.00 | 20.67 | L | C |
| ATOM | 3410 | CG1 | ILE | L | 29 | 20.844 | 9.000 | −15.291 | 1.00 | 22.28 | L | C |
| ATOM | 3411 | CD1 | ILE | L | 29 | 19.935 | 8.780 | −14.083 | 1.00 | 15.62 | L | C |
| ATOM | 3412 | C | ILE | L | 29 | 22.254 | 11.604 | −16.599 | 1.00 | 27.02 | L | C |
| ATOM | 3413 | O | ILE | L | 29 | 21.743 | 12.421 | −15.833 | 1.00 | 24.28 | L | O |
| ATOM | 3414 | N | LYS | L | 30 | 23.532 | 11.667 | −16.980 | 1.00 | 29.79 | L | N |
| ATOM | 3415 | CA | LYS | L | 30 | 24.488 | 12.667 | −16.474 | 1.00 | 28.87 | L | C |
| ATOM | 3416 | CB | LYS | L | 30 | 24.234 | 14.063 | −17.080 | 1.00 | 25.86 | L | C |
| ATOM | 3417 | CG | LYS | L | 30 | 24.806 | 14.281 | −18.468 | 1.00 | 31.53 | L | C |
| ATOM | 3418 | CD | LYS | L | 30 | 24.866 | 15.762 | −18.849 | 1.00 | 32.33 | L | C |
| ATOM | 3419 | CE | LYS | L | 30 | 26.088 | 16.454 | −18.239 | 1.00 | 32.50 | L | C |
| ATOM | 3420 | NZ | LYS | L | 30 | 26.049 | 17.913 | −18.484 | 1.00 | 33.37 | L | N |
| ATOM | 3421 | C | LYS | L | 30 | 24.526 | 12.809 | −14.965 | 1.00 | 23.69 | L | C |
| ATOM | 3422 | O | LYS | L | 30 | 24.676 | 13.913 | −14.455 | 1.00 | 25.43 | L | O |
| ATOM | 3423 | N | SER | L | 31 | 24.402 | 11.698 | −14.258 | 1.00 | 20.96 | L | N |
| ATOM | 3424 | CA | SER | L | 31 | 24.426 | 11.722 | −12.801 | 1.00 | 24.00 | L | C |
| ATOM | 3425 | CB | SER | L | 31 | 25.783 | 12.231 | −12.288 | 1.00 | 21.45 | L | C |
| ATOM | 3426 | OG | SER | L | 31 | 26.812 | 11.304 | −12.569 | 1.00 | 23.98 | L | O |
| ATOM | 3427 | C | SER | L | 31 | 23.275 | 12.518 | −12.163 | 1.00 | 22.56 | L | C |
| ATOM | 3428 | O | SER | L | 31 | 23.326 | 12.854 | −10.978 | 1.00 | 23.15 | L | O |
| ATOM | 3429 | N | TYR | L | 32 | 22.261 | 12.856 | −12.953 | 1.00 | 21.14 | L | N |
| ATOM | 3430 | CA | TYR | L | 32 | 21.101 | 13.581 | −12.424 | 1.00 | 26.25 | L | C |
| ATOM | 3431 | CB | TYR | L | 32 | 20.353 | 14.282 | −13.552 | 1.00 | 24.50 | L | C |
| ATOM | 3432 | CG | TYR | L | 32 | 21.145 | 15.340 | −14.264 | 1.00 | 28.44 | L | C |
| ATOM | 3433 | CD1 | TYR | L | 32 | 22.232 | 15.966 | −13.651 | 1.00 | 30.76 | L | C |
| ATOM | 3434 | CE1 | TYR | L | 32 | 22.951 | 16.964 | −14.307 | 1.00 | 29.77 | L | C |
| ATOM | 3435 | CD2 | TYR | L | 32 | 20.799 | 15.737 | −15.545 | 1.00 | 30.79 | L | C |
| ATOM | 3436 | CE2 | TYR | L | 32 | 21.510 | 16.733 | −16.205 | 1.00 | 31.87 | L | C |
| ATOM | 3437 | CZ | TYR | L | 32 | 22.584 | 17.340 | −15.584 | 1.00 | 28.88 | L | C |
| ATOM | 3438 | OH | TYR | L | 32 | 23.294 | 18.307 | −16.265 | 1.00 | 31.68 | L | O |
| ATOM | 3439 | C | TYR | L | 32 | 20.203 | 12.525 | −11.791 | 1.00 | 23.66 | L | C |
| ATOM | 3440 | O | TYR | L | 32 | 19.110 | 12.246 | −12.286 | 1.00 | 28.78 | L | O |
| ATOM | 3441 | N | LEU | L | 33 | 20.675 | 11.966 | −10.679 | 1.00 | 22.41 | L | N |
| ATOM | 3442 | CA | LEU | L | 33 | 20.004 | 10.890 | −9.971 | 1.00 | 21.31 | L | C |
| ATOM | 3443 | CB | LEU | L | 33 | 20.766 | 9.599 | −10.286 | 1.00 | 17.77 | L | C |
| ATOM | 3444 | CG | LEU | L | 33 | 20.264 | 8.180 | −9.984 | 1.00 | 25.99 | L | C |
| ATOM | 3445 | CD1 | LEU | L | 33 | 20.262 | 7.902 | −8.487 | 1.00 | 25.35 | L | C |
| ATOM | 3446 | CD2 | LEU | L | 33 | 18.897 | 7.968 | −10.590 | 1.00 | 17.97 | L | C |
| ATOM | 3447 | C | LEU | L | 33 | 19.962 | 11.132 | −8.449 | 1.00 | 24.74 | L | C |
| ATOM | 3448 | O | LEU | L | 33 | 20.991 | 11.339 | −7.818 | 1.00 | 22.12 | L | O |
| ATOM | 3449 | N | ASN | L | 34 | 18.768 | 11.050 | −7.867 | 1.00 | 28.54 | L | N |
| ATOM | 3450 | CA | ASN | L | 34 | 18.553 | 11.239 | −6.424 | 1.00 | 22.32 | L | C |
| ATOM | 3451 | CB | ASN | L | 34 | 17.490 | 12.314 | −6.180 | 1.00 | 20.49 | L | C |
| ATOM | 3452 | CG | ASN | L | 34 | 17.860 | 13.641 | −6.775 | 1.00 | 17.76 | L | C |
| ATOM | 3453 | OD1 | ASN | L | 34 | 19.027 | 14.015 | −6.787 | 1.00 | 25.49 | L | O |
| ATOM | 3454 | ND2 | ASN | L | 34 | 16.873 | 14.372 | −7.257 | 1.00 | 12.68 | L | N |
| ATOM | 3455 | C | ASN | L | 34 | 18.061 | 9.923 | −5.818 | 1.00 | 23.28 | L | C |
| ATOM | 3456 | O | ASN | L | 34 | 17.604 | 9.057 | −6.537 | 1.00 | 20.35 | L | O |
| ATOM | 3457 | N | TRP | L | 35 | 18.206 | 9.761 | −4.506 | 1.00 | 23.17 | L | N |
| ATOM | 3458 | CA | TRP | L | 35 | 17.762 | 8.548 | −3.815 | 1.00 | 25.44 | L | C |
| ATOM | 3459 | CB | TRP | L | 35 | 18.947 | 7.730 | −3.313 | 1.00 | 18.63 | L | C |
| ATOM | 3460 | CG | TRP | L | 35 | 19.656 | 6.937 | −4.363 | 1.00 | 20.65 | L | C |
| ATOM | 3461 | CD2 | TRP | L | 35 | 19.288 | 5.647 | −4.854 | 1.00 | 20.60 | L | C |
| ATOM | 3462 | CE2 | TRP | L | 35 | 20.279 | 5.254 | −5.784 | 1.00 | 20.55 | L | C |
| ATOM | 3463 | CE3 | TRP | L | 35 | 18.221 | 4.782 | −4.596 | 1.00 | 16.25 | L | C |
| ATOM | 3464 | CD1 | TRP | L | 35 | 20.824 | 7.266 | −4.996 | 1.00 | 23.13 | L | C |
| ATOM | 3465 | NE1 | TRP | L | 35 | 21.204 | 6.259 | −5.848 | 1.00 | 22.44 | L | N |
| ATOM | 3466 | CZ2 | TRP | L | 35 | 20.232 | 4.035 | −6.459 | 1.00 | 23.46 | L | C |
| ATOM | 3467 | CZ3 | TRP | L | 35 | 18.174 | 3.572 | −5.263 | 1.00 | 19.53 | L | C |
| ATOM | 3468 | CH2 | TRP | L | 35 | 19.172 | 3.207 | −6.187 | 1.00 | 21.99 | L | C |
| ATOM | 3469 | C | TRP | L | 35 | 16.883 | 8.946 | −2.630 | 1.00 | 25.14 | L | C |
| ATOM | 3470 | O | TRP | L | 35 | 17.181 | 9.914 | −1.920 | 1.00 | 22.30 | L | O |
| ATOM | 3471 | N | TYR | L | 36 | 15.793 | 8.208 | −2.435 | 1.00 | 24.67 | L | N |
| ATOM | 3472 | CA | TYR | L | 36 | 14.865 | 8.497 | −1.349 | 1.00 | 21.65 | L | C |
| ATOM | 3473 | CB | TYR | L | 36 | 13.572 | 9.107 | −1.868 | 1.00 | 18.13 | L | C |
| ATOM | 3474 | CG | TYR | L | 36 | 13.740 | 10.436 | −2.538 | 1.00 | 18.24 | L | C |
| ATOM | 3475 | CD1 | TYR | L | 36 | 14.177 | 10.522 | −3.851 | 1.00 | 16.76 | L | C |
| ATOM | 3476 | CE1 | TYR | L | 36 | 14.320 | 11.761 | −4.486 | 1.00 | 11.72 | L | C |
| ATOM | 3477 | CD2 | TYR | L | 36 | 13.448 | 11.616 | −1.864 | 1.00 | 14.37 | L | C |
| ATOM | 3478 | CE2 | TYR | L | 36 | 13.594 | 12.848 | −2.489 | 1.00 | 10.72 | L | C |
| ATOM | 3479 | CZ | TYR | L | 36 | 14.032 | 12.905 | −3.803 | 1.00 | 7.64 | L | C |
| ATOM | 3480 | OH | TYR | L | 36 | 14.182 | 14.124 | −4.422 | 1.00 | 18.55 | L | O |
| ATOM | 3481 | C | TYR | L | 36 | 14.512 | 7.291 | −0.531 | 1.00 | 19.36 | L | C |
| ATOM | 3482 | O | TYR | L | 36 | 14.591 | 6.155 | −1.000 | 1.00 | 26.95 | L | O |
| ATOM | 3483 | N | GLN | L | 37 | 14.130 | 7.570 | 0.706 | 1.00 | 20.33 | L | N |
| ATOM | 3484 | CA | GLN | L | 37 | 13.724 | 6.562 | 1.664 | 1.00 | 19.41 | L | C |
| ATOM | 3485 | CB | GLN | L | 37 | 14.518 | 6.703 | 2.954 | 1.00 | 12.62 | L | C |
| ATOM | 3486 | CG | GLN | L | 37 | 14.087 | 5.746 | 4.023 | 1.00 | 16.84 | L | C |
| ATOM | 3487 | CD | GLN | L | 37 | 14.714 | 6.057 | 5.381 | 1.00 | 23.54 | L | C |
| ATOM | 3488 | OE1 | GLN | L | 37 | 14.301 | 6.987 | 6.065 | 1.00 | 23.07 | L | O |
| ATOM | 3489 | NE2 | GLN | L | 37 | 15.712 | 5.269 | 5.771 | 1.00 | 24.81 | L | N |
| ATOM | 3490 | C | GLN | L | 37 | 12.270 | 6.869 | 1.969 | 1.00 | 15.72 | L | C |
| ATOM | 3491 | O | GLN | L | 37 | 11.871 | 8.030 | 2.016 | 1.00 | 13.71 | L | O |
| ATOM | 3492 | N | GLN | L | 38 | 11.477 | 5.826 | 2.138 | 1.00 | 17.20 | L | N |
| ATOM | 3493 | CA | GLN | L | 38 | 10.071 | 6.003 | 2.466 | 1.00 | 19.49 | L | C |
| ATOM | 3494 | CB | GLN | L | 38 | 9.152 | 5.948 | 1.244 | 1.00 | 15.91 | L | C |
| ATOM | 3495 | CG | GLN | L | 38 | 7.704 | 6.330 | 1.591 | 1.00 | 15.65 | L | C |
| ATOM | 3496 | CD | GLN | L | 38 | 6.814 | 6.397 | 0.372 | 1.00 | 10.79 | L | C |
| ATOM | 3497 | OE1 | GLN | L | 38 | 6.837 | 5.507 | −0.470 | 1.00 | 25.41 | L | O |
| ATOM | 3498 | NE2 | GLN | L | 38 | 6.065 | 7.472 | 0.247 | 1.00 | 10.41 | L | N |
| ATOM | 3499 | C | GLN | L | 38 | 9.647 | 4.921 | 3.392 | 1.00 | 20.98 | L | C |
| ATOM | 3500 | O | GLN | L | 38 | 10.001 | 3.739 | 3.203 | 1.00 | 12.31 | L | O |
| ATOM | 3501 | N | LYS | L | 39 | 8.920 | 5.359 | 4.410 | 1.00 | 16.73 | L | N |
| ATOM | 3502 | CA | LYS | L | 39 | 8.367 | 4.478 | 5.425 | 1.00 | 30.55 | L | C |
| ATOM | 3503 | CB | LYS | L | 39 | 8.808 | 4.930 | 6.826 | 1.00 | 28.75 | L | C |
| ATOM | 3504 | CG | LYS | L | 39 | 10.317 | 4.915 | 6.998 | 1.00 | 29.21 | L | C |
| ATOM | 3505 | CD | LYS | L | 39 | 10.709 | 4.829 | 8.446 | 1.00 | 26.38 | L | C |
| ATOM | 3506 | CE | LYS | L | 39 | 12.204 | 4.741 | 8.586 | 1.00 | 25.01 | L | C |
| ATOM | 3507 | NZ | LYS | L | 39 | 12.576 | 4.967 | 9.996 | 1.00 | 24.96 | L | N |
| ATOM | 3508 | C | LYS | L | 39 | 6.843 | 4.524 | 5.273 | 1.00 | 28.09 | L | C |
| ATOM | 3509 | O | LYS | L | 39 | 6.284 | 5.528 | 4.817 | 1.00 | 30.85 | L | O |
| ATOM | 3510 | N | PRO | L | 40 | 6.161 | 3.427 | 5.618 | 1.00 | 31.67 | L | N |
| ATOM | 3511 | CD | PRO | L | 40 | 6.770 | 2.174 | 6.103 | 1.00 | 31.20 | L | C |
| ATOM | 3512 | CA | PRO | L | 40 | 4.703 | 3.298 | 5.530 | 1.00 | 30.14 | L | C |
| ATOM | 3513 | CB | PRO | L | 40 | 4.448 | 2.027 | 6.320 | 1.00 | 35.78 | L | C |
| ATOM | 3514 | CG | PRO | L | 40 | 5.659 | 1.185 | 5.910 | 1.00 | 33.42 | L | C |
| ATOM | 3515 | C | PRO | L | 40 | 3.885 | 4.477 | 6.050 | 1.00 | 27.47 | L | C |
| ATOM | 3516 | O | PRO | L | 40 | 4.021 | 4.879 | 7.197 | 1.00 | 27.84 | L | O |
| ATOM | 3517 | N | GLY | L | 41 | 3.083 | 5.057 | 5.160 | 1.00 | 25.65 | L | N |
| ATOM | 3518 | CA | GLY | L | 41 | 2.227 | 6.175 | 5.513 | 1.00 | 22.79 | L | C |
| ATOM | 3519 | C | GLY | L | 41 | 2.887 | 7.535 | 5.631 | 1.00 | 32.81 | L | C |
| ATOM | 3520 | O | GLY | L | 41 | 2.200 | 8.516 | 5.928 | 1.00 | 33.56 | L | O |
| ATOM | 3521 | N | LYS | L | 42 | 4.197 | 7.613 | 5.382 | 1.00 | 32.94 | L | N |
| ATOM | 3522 | CA | LYS | L | 42 | 4.929 | 8.880 | 5.477 | 1.00 | 28.75 | L | C |
| ATOM | 3523 | CB | LYS | L | 42 | 6.106 | 8.732 | 6.454 | 1.00 | 29.12 | L | C |
| ATOM | 3524 | CG | LYS | L | 42 | 5.697 | 8.387 | 7.876 | 1.00 | 36.11 | L | C |
| ATOM | 3525 | CD | LYS | L | 42 | 4.794 | 9.488 | 8.433 | 1.00 | 39.46 | L | C |
| ATOM | 3526 | CE | LYS | L | 42 | 4.364 | 9.235 | 9.869 | 1.00 | 40.52 | L | C |
| ATOM | 3527 | NZ | LYS | L | 42 | 3.373 | 10.275 | 10.295 | 1.00 | 41.79 | L | N |
| ATOM | 3528 | C | LYS | L | 42 | 5.455 | 9.336 | 4.121 | 1.00 | 24.49 | L | C |
| ATOM | 3529 | O | LYS | L | 42 | 5.568 | 8.545 | 3.202 | 1.00 | 29.68 | L | O |
| ATOM | 3530 | N | ALA | L | 43 | 5.703 | 10.631 | 3.985 | 1.00 | 18.98 | L | N |
| ATOM | 3531 | CA | ALA | L | 43 | 6.264 | 11.179 | 2.761 | 1.00 | 20.62 | L | C |
| ATOM | 3532 | CB | ALA | L | 43 | 6.154 | 12.708 | 2.775 | 1.00 | 9.69 | L | C |
| ATOM | 3533 | C | ALA | L | 43 | 7.746 | 10.730 | 2.656 | 1.00 | 22.63 | L | C |
| ATOM | 3534 | O | ALA | L | 43 | 8.360 | 10.367 | 3.663 | 1.00 | 24.51 | L | O |
| ATOM | 3535 | N | PRO | L | 44 | 8.304 | 10.681 | 1.428 | 1.00 | 23.69 | L | N |
| ATOM | 3536 | CD | PRO | L | 44 | 7.624 | 10.887 | 0.135 | 1.00 | 24.06 | L | C |
| ATOM | 3537 | CA | PRO | L | 44 | 9.695 | 10.273 | 1.210 | 1.00 | 20.73 | L | C |
| ATOM | 3538 | CB | PRO | L | 44 | 9.830 | 10.316 | −0.323 | 1.00 | 18.37 | L | C |
| ATOM | 3539 | CG | PRO | L | 44 | 8.463 | 10.078 | −0.810 | 1.00 | 19.35 | L | C |
| ATOM | 3540 | C | PRO | L | 44 | 10.686 | 11.243 | 1.831 | 1.00 | 19.90 | L | C |
| ATOM | 3541 | O | PRO | L | 44 | 10.361 | 12.408 | 2.087 | 1.00 | 11.44 | L | O |
| ATOM | 3542 | N | LYS | L | 45 | 11.917 | 10.772 | 2.006 | 1.00 | 22.31 | L | N |
| ATOM | 3543 | CA | LYS | L | 45 | 12.999 | 11.595 | 2.550 | 1.00 | 26.19 | L | C |
| ATOM | 3544 | CB | LYS | L | 45 | 13.372 | 11.156 | 3.980 | 1.00 | 31.00 | L | C |
| ATOM | 3545 | CG | LYS | L | 45 | 12.764 | 12.014 | 5.076 | 1.00 | 43.97 | L | C |
| ATOM | 3546 | CD | LYS | L | 45 | 12.839 | 11.342 | 6.454 | 1.00 | 48.18 | L | C |
| ATOM | 3547 | CE | LYS | L | 45 | 11.693 | 10.348 | 6.654 | 1.00 | 50.75 | L | C |
| ATOM | 3548 | NZ | LYS | L | 45 | 11.738 | 9.631 | 7.969 | 1.00 | 55.99 | L | N |
| ATOM | 3549 | C | LYS | L | 45 | 14.222 | 11.456 | 1.653 | 1.00 | 26.08 | L | C |
| ATOM | 3550 | O | LYS | L | 45 | 14.645 | 10.336 | 1.329 | 1.00 | 19.14 | L | O |
| ATOM | 3551 | N | VAL | L | 46 | 14.788 | 12.587 | 1.249 | 1.00 | 16.72 | L | N |
| ATOM | 3552 | CA | VAL | L | 46 | 15.987 | 12.557 | 0.419 | 1.00 | 23.85 | L | C |
| ATOM | 3553 | CB | VAL | L | 46 | 16.329 | 13.975 | −0.123 | 1.00 | 25.41 | L | C |
| ATOM | 3554 | CG1 | VAL | L | 46 | 16.918 | 14.871 | 0.968 | 1.00 | 20.31 | L | C |
| ATOM | 3555 | CG2 | VAL | L | 46 | 17.241 | 13.871 | −1.310 | 1.00 | 21.07 | L | C |
| ATOM | 3556 | C | VAL | L | 46 | 17.183 | 11.979 | 1.188 | 1.00 | 18.33 | L | C |
| ATOM | 3557 | O | VAL | L | 46 | 17.288 | 12.157 | 2.384 | 1.00 | 29.18 | L | O |
| ATOM | 3558 | N | LEU | L | 47 | 18.028 | 11.221 | 0.500 | 1.00 | 22.01 | L | N |
| ATOM | 3559 | CA | LEU | L | 47 | 19.221 | 10.645 | 1.086 | 1.00 | 18.68 | L | C |
| ATOM | 3560 | CB | LEU | L | 47 | 19.192 | 9.132 | 0.994 | 1.00 | 13.51 | L | C |
| ATOM | 3561 | CG | LEU | L | 47 | 18.086 | 8.434 | 1.758 | 1.00 | 21.99 | L | C |
| ATOM | 3562 | CD1 | LEU | L | 47 | 18.005 | 6.987 | 1.277 | 1.00 | 17.12 | L | C |
| ATOM | 3563 | CD2 | LEU | L | 47 | 18.383 | 8.503 | 3.248 | 1.00 | 20.83 | L | C |
| ATOM | 3564 | C | LEU | L | 47 | 20.436 | 11.134 | 0.291 | 1.00 | 21.00 | L | C |
| ATOM | 3565 | O | LEU | L | 47 | 21.372 | 11.707 | 0.845 | 1.00 | 30.90 | L | O |
| ATOM | 3566 | N | ILE | L | 48 | 20.391 | 10.927 | −1.019 | 1.00 | 22.49 | L | N |
| ATOM | 3567 | CA | ILE | L | 48 | 21.485 | 11.277 | −1.915 | 1.00 | 15.89 | L | C |
| ATOM | 3568 | CB | ILE | L | 48 | 22.149 | 9.980 | −2.455 | 1.00 | 19.97 | L | C |
| ATOM | 3569 | CG2 | ILE | L | 48 | 23.244 | 10.303 | −3.454 | 1.00 | 21.70 | L | C |
| ATOM | 3570 | CG1 | ILE | L | 48 | 22.697 | 9.119 | −1.316 | 1.00 | 20.79 | L | C |
| ATOM | 3571 | CD1 | ILE | L | 48 | 23.882 | 9.724 | −0.583 | 1.00 | 29.53 | L | C |
| ATOM | 3572 | C | ILE | L | 48 | 21.001 | 12.070 | −3.133 | 1.00 | 23.93 | L | C |
| ATOM | 3573 | O | ILE | L | 48 | 19.928 | 11.815 | −3.681 | 1.00 | 25.69 | L | O |
| ATOM | 3574 | N | TYR | L | 49 | 21.786 | 13.055 | −3.547 | 1.00 | 24.50 | L | N |
| ATOM | 3575 | CA | TYR | L | 49 | 21.443 | 13.809 | −4.733 | 1.00 | 18.43 | L | C |
| ATOM | 3576 | CB | TYR | L | 49 | 20.903 | 15.200 | −4.396 | 1.00 | 21.01 | L | C |
| ATOM | 3577 | CG | TYR | L | 49 | 21.884 | 16.150 | −3.781 | 1.00 | 26.46 | L | C |
| ATOM | 3578 | CD1 | TYR | L | 49 | 22.273 | 16.021 | −2.444 | 1.00 | 32.43 | L | C |
| ATOM | 3579 | CE1 | TYR | L | 49 | 23.163 | 16.926 | −1.865 | 1.00 | 22.08 | L | C |
| ATOM | 3580 | CD2 | TYR | L | 49 | 22.405 | 17.209 | −4.517 | 1.00 | 26.33 | L | C |
| ATOM | 3581 | CE2 | TYR | L | 49 | 23.285 | 18.116 | −3.943 | 1.00 | 24.23 | L | C |
| ATOM | 3582 | CZ | TYR | L | 49 | 23.664 | 17.965 | −2.620 | 1.00 | 22.18 | L | C |
| ATOM | 3583 | OH | TYR | L | 49 | 24.583 | 18.838 | −2.075 | 1.00 | 29.75 | L | O |
| ATOM | 3584 | C | TYR | L | 49 | 22.671 | 13.854 | −5.642 | 1.00 | 23.55 | L | C |
| ATOM | 3585 | O | TYR | L | 49 | 23.767 | 13.496 | −5.217 | 1.00 | 22.70 | L | O |
| ATOM | 3586 | N | TYR | L | 50 | 22.455 | 14.183 | −6.917 | 1.00 | 21.10 | L | N |
| ATOM | 3587 | CA | TYR | L | 50 | 23.511 | 14.261 | −7.920 | 1.00 | 22.32 | L | C |
| ATOM | 3588 | CB | TYR | L | 50 | 24.316 | 15.555 | −7.767 | 1.00 | 30.29 | L | C |
| ATOM | 3589 | CG | TYR | L | 50 | 25.235 | 15.870 | −8.931 | 1.00 | 29.49 | L | C |
| ATOM | 3590 | CD1 | TYR | L | 50 | 24.738 | 16.003 | −10.234 | 1.00 | 31.32 | L | C |
| ATOM | 3591 | CE1 | TYR | L | 50 | 25.593 | 16.253 | −11.323 | 1.00 | 25.87 | L | C |
| ATOM | 3592 | CD2 | TYR | L | 50 | 26.603 | 15.999 | −8.735 | 1.00 | 26.49 | L | C |
| ATOM | 3593 | CE2 | TYR | L | 50 | 27.466 | 16.246 | −9.806 | 1.00 | 31.66 | L | C |
| ATOM | 3594 | CZ | TYR | L | 50 | 26.959 | 16.369 | −11.098 | 1.00 | 30.62 | L | C |
| ATOM | 3595 | OH | TYR | L | 50 | 27.833 | 16.574 | −12.144 | 1.00 | 29.62 | L | O |
| ATOM | 3596 | C | TYR | L | 50 | 24.396 | 13.018 | −7.925 | 1.00 | 22.67 | L | C |
| ATOM | 3597 | O | TYR | L | 50 | 25.630 | 13.089 | −8.029 | 1.00 | 25.09 | L | O |
| ATOM | 3598 | N | ALA | L | 51 | 23.725 | 11.880 | −7.763 | 1.00 | 20.12 | L | N |
| ATOM | 3599 | CA | ALA | L | 51 | 24.336 | 10.559 | −7.771 | 1.00 | 23.28 | L | C |
| ATOM | 3600 | CB | ALA | L | 51 | 25.051 | 10.319 | −9.097 | 1.00 | 26.17 | L | C |
| ATOM | 3601 | C | ALA | L | 51 | 25.257 | 10.204 | −6.638 | 1.00 | 22.27 | L | C |
| ATOM | 3602 | O | ALA | L | 51 | 25.265 | 9.051 | −6.214 | 1.00 | 19.78 | L | O |
| ATOM | 3603 | N | THR | L | 52 | 26.047 | 11.170 | −6.163 | 1.00 | 22.12 | L | N |
| ATOM | 3604 | CA | THR | L | 52 | 27.007 | 10.886 | −5.107 | 1.00 | 23.66 | L | C |
| ATOM | 3605 | CB | THR | L | 52 | 28.434 | 10.816 | −5.702 | 1.00 | 29.10 | L | C |
| ATOM | 3606 | OG1 | THR | L | 52 | 28.824 | 12.126 | −6.140 | 1.00 | 33.56 | L | O |
| ATOM | 3607 | CG2 | THR | L | 52 | 28.483 | 9.850 | −6.901 | 1.00 | 22.13 | L | C |
| ATOM | 3608 | C | THR | L | 52 | 27.039 | 11.805 | −3.869 | 1.00 | 26.42 | L | C |
| ATOM | 3609 | O | THR | L | 52 | 27.797 | 11.534 | −2.934 | 1.00 | 23.16 | L | O |
| ATOM | 3610 | N | SER | L | 53 | 26.256 | 12.883 | −3.852 | 1.00 | 20.29 | L | N |
| ATOM | 3611 | CA | SER | L | 53 | 26.248 | 13.778 | −2.678 | 1.00 | 22.16 | L | C |
| ATOM | 3612 | CB | SER | L | 53 | 25.933 | 15.214 | −3.071 | 1.00 | 18.52 | L | C |
| ATOM | 3613 | OG | SER | L | 53 | 26.876 | 15.690 | −4.009 | 1.00 | 38.79 | L | O |
| ATOM | 3614 | C | SER | L | 53 | 25.292 | 13.388 | −1.552 | 1.00 | 25.35 | L | C |
| ATOM | 3615 | O | SER | L | 53 | 24.089 | 13.152 | −1.770 | 1.00 | 24.36 | L | O |
| ATOM | 3616 | N | LEU | L | 54 | 25.843 | 13.334 | −0.340 | 1.00 | 29.14 | L | N |
| ATOM | 3617 | CA | LEU | L | 54 | 25.078 | 13.022 | 0.871 | 1.00 | 25.39 | L | C |
| ATOM | 3618 | CB | LEU | L | 54 | 26.029 | 12.723 | 2.025 | 1.00 | 25.12 | L | C |
| ATOM | 3619 | CG | LEU | L | 54 | 25.374 | 12.391 | 3.361 | 1.00 | 27.62 | L | C |
| ATOM | 3620 | CD1 | LEU | L | 54 | 24.531 | 11.129 | 3.202 | 1.00 | 23.72 | L | C |
| ATOM | 3621 | CD2 | LEU | L | 54 | 26.450 | 12.181 | 4.415 | 1.00 | 20.02 | L | C |
| ATOM | 3622 | C | LEU | L | 54 | 24.308 | 14.291 | 1.198 | 1.00 | 27.19 | L | C |
| ATOM | 3623 | O | LEU | L | 54 | 24.907 | 15.359 | 1.333 | 1.00 | 27.73 | L | O |
| ATOM | 3624 | N | ALA | L | 55 | 22.988 | 14.193 | 1.290 | 1.00 | 24.10 | L | N |
| ATOM | 3625 | CA | ALA | L | 55 | 22.168 | 15.369 | 1.579 | 1.00 | 24.34 | L | C |
| ATOM | 3626 | CB | ALA | L | 55 | 20.686 | 15.072 | 1.303 | 1.00 | 22.43 | L | C |
| ATOM | 3627 | C | ALA | L | 55 | 22.380 | 15.784 | 3.027 | 1.00 | 24.59 | L | C |
| ATOM | 3628 | O | ALA | L | 55 | 22.753 | 14.958 | 3.855 | 1.00 | 18.62 | L | O |
| ATOM | 3629 | N | GLU | L | 56 | 22.122 | 17.051 | 3.334 | 1.00 | 25.67 | L | N |
| ATOM | 3630 | CA | GLU | L | 56 | 22.323 | 17.548 | 4.679 | 1.00 | 31.17 | L | C |
| ATOM | 3631 | CB | GLU | L | 56 | 22.080 | 19.055 | 4.725 | 1.00 | 35.89 | L | C |
| ATOM | 3632 | CG | GLU | L | 56 | 22.048 | 19.635 | 6.122 | 1.00 | 46.70 | L | C |
| ATOM | 3633 | CD | GLU | L | 56 | 22.788 | 20.944 | 6.214 | 1.00 | 55.09 | L | C |
| ATOM | 3634 | OE1 | GLU | L | 56 | 24.039 | 20.903 | 6.289 | 1.00 | 60.61 | L | O |
| ATOM | 3635 | OE2 | GLU | L | 56 | 22.127 | 22.008 | 6.213 | 1.00 | 58.04 | L | O |
| ATOM | 3636 | C | GLU | L | 56 | 21.495 | 16.821 | 5.737 | 1.00 | 32.93 | L | C |
| ATOM | 3637 | O | GLU | L | 56 | 20.321 | 16.523 | 5.527 | 1.00 | 36.52 | L | O |
| ATOM | 3638 | N | GLY | L | 57 | 22.154 | 16.480 | 6.842 | 1.00 | 30.32 | L | N |
| ATOM | 3639 | CA | GLY | L | 57 | 21.498 | 15.807 | 7.947 | 1.00 | 27.34 | L | C |
| ATOM | 3640 | C | GLY | L | 57 | 21.302 | 14.321 | 7.786 | 1.00 | 26.71 | L | C |
| ATOM | 3641 | O | GLY | L | 57 | 20.772 | 13.669 | 8.673 | 1.00 | 33.94 | L | O |
| ATOM | 3642 | N | VAL | L | 58 | 21.739 | 13.774 | 6.663 | 1.00 | 22.48 | L | N |
| ATOM | 3643 | CA | VAL | L | 58 | 21.571 | 12.350 | 6.416 | 1.00 | 19.32 | L | C |
| ATOM | 3644 | CB | VAL | L | 58 | 21.424 | 12.085 | 4.876 | 1.00 | 23.06 | L | C |
| ATOM | 3645 | CG1 | VAL | L | 58 | 21.346 | 10.595 | 4.554 | 1.00 | 6.82 | L | C |
| ATOM | 3646 | CG2 | VAL | L | 58 | 20.196 | 12.826 | 4.334 | 1.00 | 12.50 | L | C |
| ATOM | 3647 | C | VAL | L | 58 | 22.727 | 11.545 | 7.000 | 1.00 | 17.15 | L | C |
| ATOM | 3648 | O | VAL | L | 58 | 23.886 | 11.929 | 6.865 | 1.00 | 18.33 | L | O |
| ATOM | 3649 | N | PRO | L | 59 | 22.418 | 10.455 | 7.722 | 1.00 | 18.83 | L | N |
| ATOM | 3650 | CD | PRO | L | 59 | 21.074 | 9.990 | 8.109 | 1.00 | 15.08 | L | C |
| ATOM | 3651 | CA | PRO | L | 59 | 23.452 | 9.603 | 8.320 | 1.00 | 19.83 | L | C |
| ATOM | 3652 | CB | PRO | L | 59 | 22.654 | 8.400 | 8.812 | 1.00 | 16.59 | L | C |
| ATOM | 3653 | CG | PRO | L | 59 | 21.379 | 9.013 | 9.243 | 1.00 | 11.61 | L | C |
| ATOM | 3654 | C | PRO | L | 59 | 24.471 | 9.186 | 7.255 | 1.00 | 29.12 | L | C |
| ATOM | 3655 | O | PRO | L | 59 | 24.119 | 8.748 | 6.152 | 1.00 | 28.09 | L | O |
| ATOM | 3656 | N | SER | L | 60 | 25.738 | 9.308 | 7.616 | 1.00 | 29.14 | L | N |
| ATOM | 3657 | CA | SER | L | 60 | 26.840 | 9.009 | 6.723 | 1.00 | 23.16 | L | C |
| ATOM | 3658 | CB | SER | L | 60 | 28.151 | 9.413 | 7.397 | 1.00 | 25.36 | L | C |
| ATOM | 3659 | OG | SER | L | 60 | 28.396 | 8.593 | 8.537 | 1.00 | 24.87 | L | O |
| ATOM | 3660 | C | SER | L | 60 | 26.929 | 7.559 | 6.240 | 1.00 | 23.01 | L | C |
| ATOM | 3661 | O | SER | L | 60 | 27.722 | 7.266 | 5.355 | 1.00 | 14.47 | L | O |
| ATOM | 3662 | N | ARG | L | 61 | 26.143 | 6.652 | 6.814 | 1.00 | 19.74 | L | N |
| ATOM | 3663 | CA | ARG | L | 61 | 26.214 | 5.250 | 6.380 | 1.00 | 17.33 | L | C |
| ATOM | 3664 | CB | ARG | L | 61 | 25.567 | 4.308 | 7.390 | 1.00 | 12.56 | L | C |
| ATOM | 3665 | CG | ARG | L | 61 | 24.087 | 4.550 | 7.640 | 1.00 | 9.45 | L | C |
| ATOM | 3666 | CD | ARG | L | 61 | 23.551 | 3.493 | 8.597 | 1.00 | 13.71 | L | C |
| ATOM | 3667 | NE | ARG | L | 61 | 22.197 | 3.811 | 9.024 | 1.00 | 20.44 | L | N |
| ATOM | 3668 | CZ | ARG | L | 61 | 21.877 | 4.847 | 9.791 | 1.00 | 18.85 | L | C |
| ATOM | 3669 | NH1 | ARG | L | 61 | 22.821 | 5.678 | 10.213 | 1.00 | 23.65 | L | N |
| ATOM | 3670 | NH2 | ARG | L | 61 | 20.624 | 5.022 | 10.179 | 1.00 | 17.74 | L | N |
| ATOM | 3671 | C | ARG | L | 61 | 25.577 | 5.075 | 5.001 | 1.00 | 22.67 | L | C |
| ATOM | 3672 | O | ARG | L | 61 | 25.721 | 4.023 | 4.362 | 1.00 | 22.15 | L | O |
| ATOM | 3673 | N | PHE | L | 62 | 24.870 | 6.111 | 4.563 | 1.00 | 10.17 | L | N |
| ATOM | 3674 | CA | PHE | L | 62 | 24.237 | 6.111 | 3.257 | 1.00 | 23.06 | L | C |
| ATOM | 3675 | CB | PHE | L | 62 | 22.921 | 6.924 | 3.267 | 1.00 | 12.05 | L | C |
| ATOM | 3676 | CG | PHE | L | 62 | 21.855 | 6.325 | 4.097 | 1.00 | 16.73 | L | C |
| ATOM | 3677 | CD1 | PHE | L | 62 | 21.035 | 5.320 | 3.572 | 1.00 | 22.85 | L | C |
| ATOM | 3678 | CD2 | PHE | L | 62 | 21.668 | 6.743 | 5.418 | 1.00 | 13.08 | L | C |
| ATOM | 3679 | CE1 | PHE | L | 62 | 20.037 | 4.730 | 4.357 | 1.00 | 22.20 | L | C |
| ATOM | 3680 | CE2 | PHE | L | 62 | 20.701 | 6.179 | 6.202 | 1.00 | 15.34 | L | C |
| ATOM | 3681 | CZ | PHE | L | 62 | 19.868 | 5.159 | 5.678 | 1.00 | 23.42 | L | C |
| ATOM | 3682 | C | PHE | L | 62 | 25.196 | 6.772 | 2.279 | 1.00 | 23.15 | L | C |
| ATOM | 3683 | O | PHE | L | 62 | 25.719 | 7.863 | 2.549 | 1.00 | 26.48 | L | O |
| ATOM | 3684 | N | SER | L | 63 | 25.443 | 6.115 | 1.153 | 1.00 | 16.35 | L | N |
| ATOM | 3685 | CA | SER | L | 63 | 26.299 | 6.718 | 0.146 | 1.00 | 20.31 | L | C |
| ATOM | 3686 | CB | SER | L | 63 | 27.779 | 6.410 | 0.390 | 1.00 | 22.57 | L | C |
| ATOM | 3687 | OG | SER | L | 63 | 28.052 | 5.042 | 0.229 | 1.00 | 28.48 | L | O |
| ATOM | 3688 | C | SER | L | 63 | 25.896 | 6.311 | −1.256 | 1.00 | 22.09 | L | C |
| ATOM | 3689 | O | SER | L | 63 | 25.390 | 5.206 | −1.496 | 1.00 | 16.83 | L | O |
| ATOM | 3690 | N | GLY | L | 64 | 26.070 | 7.243 | −2.179 | 1.00 | 25.03 | L | N |
| ATOM | 3691 | CA | GLY | L | 64 | 25.737 | 6.966 | −3.560 | 1.00 | 29.73 | L | C |
| ATOM | 3692 | C | GLY | L | 64 | 26.966 | 6.915 | −4.449 | 1.00 | 30.91 | L | C |
| ATOM | 3693 | O | GLY | L | 64 | 27.976 | 7.559 | −4.178 | 1.00 | 34.93 | L | O |
| ATOM | 3694 | N | SER | L | 65 | 26.906 | 6.066 | −5.464 | 1.00 | 32.07 | L | N |
| ATOM | 3695 | CA | SER | L | 65 | 27.982 | 5.955 | −6.431 | 1.00 | 31.54 | L | C |
| ATOM | 3696 | CB | SER | L | 65 | 29.021 | 4.907 | −6.014 | 1.00 | 32.31 | L | C |
| ATOM | 3697 | OG | SER | L | 65 | 28.420 | 3.677 | −5.681 | 1.00 | 38.61 | L | O |
| ATOM | 3698 | C | SER | L | 65 | 27.417 | 5.661 | −7.822 | 1.00 | 33.01 | L | C |
| ATOM | 3699 | O | SER | L | 65 | 26.235 | 5.320 | −7.978 | 1.00 | 22.60 | L | O |
| ATOM | 3700 | N | GLY | L | 66 | 28.252 | 5.880 | −8.838 | 1.00 | 34.64 | L | N |
| ATOM | 3701 | CA | GLY | L | 66 | 27.838 | 5.633 | −10.202 | 1.00 | 31.12 | L | C |
| ATOM | 3702 | C | GLY | L | 66 | 27.820 | 6.827 | −11.131 | 1.00 | 30.45 | L | C |
| ATOM | 3703 | O | GLY | L | 66 | 28.003 | 7.973 | −10.724 | 1.00 | 27.30 | L | O |
| ATOM | 3704 | N | SER | L | 67 | 27.602 | 6.530 | −12.405 | 1.00 | 29.09 | L | N |
| ATOM | 3705 | CA | SER | L | 67 | 27.552 | 7.537 | −13.452 | 1.00 | 32.09 | L | C |
| ATOM | 3706 | CB | SER | L | 67 | 28.944 | 8.149 | −13.687 | 1.00 | 32.68 | L | C |
| ATOM | 3707 | OG | SER | L | 67 | 29.890 | 7.154 | −14.062 | 1.00 | 34.98 | L | O |
| ATOM | 3708 | C | SER | L | 67 | 27.067 | 6.829 | −14.704 | 1.00 | 29.55 | L | C |
| ATOM | 3709 | O | SER | L | 67 | 27.024 | 5.602 | −14.753 | 1.00 | 27.95 | L | O |
| ATOM | 3710 | N | GLY | L | 68 | 26.736 | 7.603 | −15.726 | 1.00 | 29.87 | L | N |
| ATOM | 3711 | CA | GLY | L | 68 | 26.252 | 7.006 | −16.952 | 1.00 | 30.09 | L | C |
| ATOM | 3712 | C | GLY | L | 68 | 24.839 | 6.488 | −16.767 | 1.00 | 31.47 | L | C |
| ATOM | 3713 | O | GLY | L | 68 | 23.903 | 7.271 | −16.600 | 1.00 | 32.96 | L | O |
| ATOM | 3714 | N | THR | L | 69 | 24.684 | 5.168 | −16.791 | 1.00 | 24.13 | L | N |
| ATOM | 3715 | CA | THR | L | 69 | 23.376 | 4.549 | −16.625 | 1.00 | 24.44 | L | C |
| ATOM | 3716 | CB | THR | L | 69 | 23.021 | 3.636 | −17.832 | 1.00 | 23.34 | L | C |
| ATOM | 3717 | OG1 | THR | L | 69 | 23.946 | 2.544 | −17.909 | 1.00 | 22.34 | L | O |
| ATOM | 3718 | CG2 | THR | L | 69 | 23.057 | 4.411 | −19.116 | 1.00 | 24.03 | L | C |
| ATOM | 3719 | C | THR | L | 69 | 23.310 | 3.688 | −15.365 | 1.00 | 26.40 | L | C |
| ATOM | 3720 | O | THR | L | 69 | 22.225 | 3.270 | −14.955 | 1.00 | 25.50 | L | O |
| ATOM | 3721 | N | ASP | L | 70 | 24.462 | 3.451 | −14.738 | 1.00 | 18.33 | L | N |
| ATOM | 3722 | CA | ASP | L | 70 | 24.526 | 2.597 | −13.555 | 1.00 | 27.77 | L | C |
| ATOM | 3723 | CB | ASP | L | 70 | 25.615 | 1.539 | −13.743 | 1.00 | 28.50 | L | C |
| ATOM | 3724 | CG | ASP | L | 70 | 25.594 | 0.917 | −15.139 | 1.00 | 37.20 | L | C |
| ATOM | 3725 | OD1 | ASP | L | 70 | 24.682 | 0.108 | −15.433 | 1.00 | 34.23 | L | O |
| ATOM | 3726 | OD2 | ASP | L | 70 | 26.489 | 1.257 | −15.948 | 1.00 | 42.39 | L | O |
| ATOM | 3727 | C | ASP | L | 70 | 24.760 | 3.352 | −12.253 | 1.00 | 27.10 | L | C |
| ATOM | 3728 | O | ASP | L | 70 | 25.762 | 4.041 | −12.090 | 1.00 | 33.82 | L | O |
| ATOM | 3729 | N | TYR | L | 71 | 23.848 | 3.186 | −11.307 | 1.00 | 19.53 | L | N |
| ATOM | 3730 | CA | TYR | L | 71 | 23.959 | 3.878 | −10.045 | 1.00 | 22.26 | L | C |
| ATOM | 3731 | CB | TYR | L | 71 | 22.955 | 5.029 | −9.983 | 1.00 | 17.96 | L | C |
| ATOM | 3732 | CG | TYR | L | 71 | 23.226 | 6.056 | −11.040 | 1.00 | 24.30 | L | C |
| ATOM | 3733 | CD1 | TYR | L | 71 | 22.754 | 5.880 | −12.347 | 1.00 | 21.22 | L | C |
| ATOM | 3734 | CE1 | TYR | L | 71 | 23.069 | 6.782 | −13.351 | 1.00 | 22.51 | L | C |
| ATOM | 3735 | CD2 | TYR | L | 71 | 24.014 | 7.165 | −10.766 | 1.00 | 21.23 | L | C |
| ATOM | 3736 | CE2 | TYR | L | 71 | 24.330 | 8.074 | −11.765 | 1.00 | 24.84 | L | C |
| ATOM | 3737 | CZ | TYR | L | 71 | 23.856 | 7.879 | −13.057 | 1.00 | 25.28 | L | C |
| ATOM | 3738 | OH | TYR | L | 71 | 24.177 | 8.781 | −14.049 | 1.00 | 32.28 | L | O |
| ATOM | 3739 | C | TYR | L | 71 | 23.709 | 2.942 | −8.907 | 1.00 | 21.55 | L | C |
| ATOM | 3740 | O | TYR | L | 71 | 22.994 | 1.962 | −9.061 | 1.00 | 30.10 | L | O |
| ATOM | 3741 | N | THR | L | 72 | 24.367 | 3.186 | −7.782 | 1.00 | 21.16 | L | N |
| ATOM | 3742 | CA | THR | L | 72 | 24.123 | 2.355 | −6.618 | 1.00 | 25.86 | L | C |
| ATOM | 3743 | CB | THR | L | 72 | 25.147 | 1.193 | −6.446 | 1.00 | 28.16 | L | C |
| ATOM | 3744 | OG1 | THR | L | 72 | 26.300 | 1.650 | −5.740 | 1.00 | 32.24 | L | O |
| ATOM | 3745 | CG2 | THR | L | 72 | 25.566 | 0.631 | −7.779 | 1.00 | 29.80 | L | C |
| ATOM | 3746 | C | THR | L | 72 | 24.040 | 3.150 | −5.320 | 1.00 | 24.15 | L | C |
| ATOM | 3747 | O | THR | L | 72 | 24.745 | 4.154 | −5.118 | 1.00 | 20.51 | L | O |
| ATOM | 3748 | N | LEU | L | 73 | 23.094 | 2.730 | −4.488 | 1.00 | 23.92 | L | N |
| ATOM | 3749 | CA | LEU | L | 73 | 22.879 | 3.299 | −3.165 | 1.00 | 19.11 | L | C |
| ATOM | 3750 | CB | LEU | L | 73 | 21.390 | 3.480 | −2.873 | 1.00 | 20.99 | L | C |
| ATOM | 3751 | CG | LEU | L | 73 | 21.116 | 3.826 | −1.401 | 1.00 | 25.18 | L | C |
| ATOM | 3752 | CD1 | LEU | L | 73 | 21.713 | 5.214 | −1.014 | 1.00 | 13.97 | L | C |
| ATOM | 3753 | CD2 | LEU | L | 73 | 19.619 | 3.769 | −1.174 | 1.00 | 17.46 | L | C |
| ATOM | 3754 | C | LEU | L | 73 | 23.423 | 2.261 | −2.204 | 1.00 | 22.44 | L | C |
| ATOM | 3755 | O | LEU | L | 73 | 23.017 | 1.098 | −2.258 | 1.00 | 21.13 | L | O |
| ATOM | 3756 | N | THR | L | 74 | 24.359 | 2.662 | −1.349 | 1.00 | 27.57 | L | N |
| ATOM | 3757 | CA | THR | L | 74 | 24.935 | 1.735 | −0.378 | 1.00 | 26.12 | L | C |
| ATOM | 3758 | CB | THR | L | 74 | 26.484 | 1.589 | −0.553 | 1.00 | 31.09 | L | C |
| ATOM | 3759 | OG1 | THR | L | 74 | 26.789 | 1.148 | −1.884 | 1.00 | 27.24 | L | O |
| ATOM | 3760 | CG2 | THR | L | 74 | 27.047 | 0.574 | 0.439 | 1.00 | 18.81 | L | C |
| ATOM | 3761 | C | THR | L | 74 | 24.648 | 2.175 | 1.051 | 1.00 | 24.71 | L | C |
| ATOM | 3762 | O | THR | L | 74 | 24.725 | 3.363 | 1.374 | 1.00 | 28.22 | L | O |
| ATOM | 3763 | N | ILE | L | 75 | 24.226 | 1.221 | 1.874 | 1.00 | 21.36 | L | N |
| ATOM | 3764 | CA | ILE | L | 75 | 23.972 | 1.464 | 3.296 | 1.00 | 20.68 | L | C |
| ATOM | 3765 | CB | ILE | L | 75 | 22.553 | 1.005 | 3.734 | 1.00 | 22.37 | L | C |
| ATOM | 3766 | CG2 | ILE | L | 75 | 22.332 | 1.362 | 5.240 | 1.00 | 15.53 | L | C |
| ATOM | 3767 | CG1 | ILE | L | 75 | 21.491 | 1.676 | 2.849 | 1.00 | 11.46 | L | C |
| ATOM | 3768 | CD1 | ILE | L | 75 | 20.060 | 1.337 | 3.200 | 1.00 | 17.68 | L | C |
| ATOM | 3769 | C | ILE | L | 75 | 25.039 | 0.614 | 3.992 | 1.00 | 20.45 | L | C |
| ATOM | 3770 | O | ILE | L | 75 | 24.894 | −0.599 | 4.081 | 1.00 | 15.20 | L | O |
| ATOM | 3771 | N | SER | L | 76 | 26.105 | 1.251 | 4.476 | 1.00 | 22.43 | L | N |
| ATOM | 3772 | CA | SER | L | 76 | 27.229 | 0.525 | 5.081 | 1.00 | 27.24 | L | C |
| ATOM | 3773 | CB | SER | L | 76 | 28.450 | 1.437 | 5.230 | 1.00 | 27.59 | L | C |
| ATOM | 3774 | OG | SER | L | 76 | 28.294 | 2.366 | 6.291 | 1.00 | 36.14 | L | O |
| ATOM | 3775 | C | SER | L | 76 | 27.023 | −0.263 | 6.375 | 1.00 | 34.31 | L | C |
| ATOM | 3776 | O | SER | L | 76 | 27.735 | −1.250 | 6.613 | 1.0O | 38.03 | L | O |
| ATOM | 3777 | N | SER | L | 77 | 26.088 | 0.162 | 7.221 | 1.00 | 29.03 | L | N |
| ATOM | 3778 | CA | SER | L | 77 | 25.844 | −0.555 | 8.466 | 1.00 | 29.73 | L | C |
| ATOM | 3779 | CB | SER | L | 77 | 26.638 | 0.070 | 9.617 | 1.00 | 27.77 | L | C |
| ATOM | 3780 | OG | SER | L | 77 | 26.820 | −0.881 | 10.649 | 1.00 | 36.16 | L | O |
| ATOM | 3781 | C | SER | L | 77 | 24.357 | −0.471 | 8.733 | 1.00 | 28.69 | L | C |
| ATOM | 3782 | O | SER | L | 77 | 23.886 | 0.445 | 9.405 | 1.00 | 35.46 | L | O |
| ATOM | 3783 | N | LEU | L | 78 | 23.622 | −1.437 | 8.197 | 1.00 | 26.30 | L | N |
| ATOM | 3784 | CA | LEU | L | 78 | 22.171 | −1.456 | 8.322 | 1.00 | 24.65 | L | C |
| ATOM | 3785 | CB | LEU | L | 78 | 21.584 | −2.681 | 7.617 | 1.00 | 18.78 | L | C |
| ATOM | 3786 | CG | LEU | L | 78 | 20.082 | −2.617 | 7.358 | 1.00 | 22.74 | L | C |
| ATOM | 3787 | CD1 | LEU | L | 78 | 19.763 | −1.516 | 6.365 | 1.00 | 16.54 | L | C |
| ATOM | 3788 | CD2 | LEU | L | 78 | 19.577 | −3.954 | 6.840 | 1.00 | 20.54 | L | C |
| ATOM | 3789 | C | LEU | L | 78 | 21.654 | −1.354 | 9.754 | 1.00 | 20.98 | L | C |
| ATOM | 3790 | O | LEU | L | 78 | 21.937 | −2.205 | 10.588 | 1.00 | 20.94 | L | O |
| ATOM | 3791 | N | GLN | L | 79 | 20.904 | −0.284 | 10.009 | 1.00 | 20.24 | L | N |
| ATOM | 3792 | CA | GLN | L | 79 | 20.305 | −0.002 | 11.313 | 1.00 | 24.23 | L | C |
| ATOM | 3793 | CB | GLN | L | 79 | 20.576 | 1.466 | 11.666 | 1.00 | 26.74 | L | C |
| ATOM | 3794 | CG | GLN | L | 79 | 22.071 | 1.804 | 11.777 | 1.00 | 23.12 | L | C |
| ATOM | 3795 | CD | GLN | L | 79 | 22.780 | 0.923 | 12.793 | 1.00 | 23.14 | L | C |
| ATOM | 3796 | OE1 | GLN | L | 79 | 22.514 | 1.010 | 13.986 | 1.00 | 20.44 | L | O |
| ATOM | 3797 | NE2 | GLN | L | 79 | 23.655 | 0.035 | 12.315 | 1.00 | 29.47 | L | N |
| ATOM | 3798 | C | GLN | L | 79 | 18.785 | −0.323 | 11.325 | 1.00 | 23.15 | L | C |
| ATOM | 3799 | O | GLN | L | 79 | 18.142 | −0.294 | 10.277 | 1.00 | 27.29 | L | O |
| ATOM | 3800 | N | PRO | L | 80 | 18.205 | −0.663 | 12.501 | 1.00 | 22.73 | L | N |
| ATOM | 3801 | CD | PRO | L | 80 | 18.863 | −0.804 | 13.818 | 1.00 | 21.11 | L | C |
| ATOM | 3802 | CA | PRO | L | 80 | 16.767 | −0.989 | 12.598 | 1.00 | 19.90 | L | C |
| ATOM | 3803 | CB | PRO | L | 80 | 16.520 | −1.018 | 14.101 | 1.00 | 17.43 | L | C |
| ATOM | 3804 | CG | PRO | L | 80 | 17.824 | −1.599 | 14.624 | 1.00 | 18.34 | L | C |
| ATOM | 3805 | C | PRO | L | 80 | 15.870 | 0.022 | 11.911 | 1.00 | 21.29 | L | C |
| ATOM | 3806 | O | PRO | L | 80 | 14.938 | −0.345 | 11.205 | 1.00 | 27.08 | L | O |
| ATOM | 3807 | N | GLU | L | 81 | 16.205 | 1.294 | 12.070 | 1.00 | 19.93 | L | N |
| ATOM | 3808 | CA | GLU | L | 81 | 15.458 | 2.380 | 11.464 | 1.00 | 17.17 | L | C |
| ATOM | 3809 | CB | GLU | L | 81 | 15.822 | 3.713 | 12.150 | 1.00 | 15.37 | L | C |
| ATOM | 3810 | CG | GLU | L | 81 | 17.331 | 4.076 | 12.157 | 1.00 | 19.75 | L | C |
| ATOM | 3811 | CD | GLU | L | 81 | 18.109 | 3.559 | 13.386 | 1.00 | 22.90 | L | C |
| ATOM | 3812 | OE1 | GLU | L | 81 | 17.839 | 2.437 | 13.867 | 1.00 | 21.15 | L | O |
| ATOM | 3813 | OE2 | GLU | L | 81 | 19.014 | 4.282 | 13.868 | 1.00 | 21.13 | L | O |
| ATOM | 3814 | C | GLU | L | 81 | 15.665 | 2.475 | 9.943 | 1.00 | 20.84 | L | C |
| ATOM | 3815 | O | GLU | L | 81 | 15.037 | 3.294 | 9.288 | 1.00 | 21.14 | L | O |
| ATOM | 3816 | N | ASP | L | 82 | 16.546 | 1.650 | 9.383 | 1.00 | 19.73 | L | N |
| ATOM | 3817 | CA | ASP | L | 82 | 16.793 | 1.674 | 7.939 | 1.00 | 21.60 | L | C |
| ATOM | 3818 | CB | ASP | L | 82 | 18.242 | 1.293 | 7.628 | 1.00 | 18.79 | L | C |
| ATOM | 3819 | CG | ASP | L | 82 | 19.236 | 2.307 | 8.146 | 1.00 | 24.18 | L | C |
| ATOM | 3820 | OD1 | ASP | L | 82 | 18.870 | 3.495 | 8.254 | 1.00 | 18.99 | L | O |
| ATOM | 3821 | OD2 | ASP | L | 82 | 20.383 | 1.912 | 8.449 | 1.00 | 17.83 | L | O |
| ATOM | 3822 | C | ASP | L | 82 | 15.842 | 0.776 | 7.132 | 1.00 | 23.67 | L | C |
| ATOM | 3823 | O | ASP | L | 82 | 15.829 | 0.831 | 5.906 | 1.00 | 27.88 | L | O |
| ATOM | 3824 | N | PHE | L | 83 | 15.107 | −0.098 | 7.812 | 1.00 | 21.03 | L | N |
| ATOM | 3825 | CA | PHE | L | 83 | 14.158 | −0.978 | 7.140 | 1.00 | 21.95 | L | C |
| ATOM | 3826 | CB | PHE | L | 83 | 13.662 | −2.068 | 8.086 | 1.00 | 12.49 | L | C |
| ATOM | 3827 | CG | PHE | L | 83 | 14.720 | −3.062 | 8.423 | 1.00 | 19.54 | L | C |
| ATOM | 3828 | CD1 | PHE | L | 83 | 15.099 | −4.026 | 7.493 | 1.00 | 14.69 | L | C |
| ATOM | 3829 | CD2 | PHE | L | 83 | 15.387 | −3.004 | 9.651 | 1.00 | 20.19 | L | C |
| ATOM | 3830 | CE1 | PHE | L | 83 | 16.138 | −4.930 | 7.775 | 1.00 | 21.34 | L | C |
| ATOM | 3831 | CE2 | PHE | L | 83 | 16.427 | −3.899 | 9.948 | 1.00 | 20.86 | L | C |
| ATOM | 3832 | CZ | PHE | L | 83 | 16.808 | −4.864 | 9.012 | 1.00 | 21.36 | L | C |
| ATOM | 3833 | C | PHE | L | 83 | 13.024 | −0.116 | 6.623 | 1.00 | 23.92 | L | C |
| ATOM | 3834 | O | PHE | L | 83 | 12.257 | 0.473 | 7.404 | 1.00 | 18.68 | L | O |
| ATOM | 3835 | N | ALA | L | 84 | 12.948 | −0.042 | 5.293 | 1.00 | 21.04 | L | N |
| ATOM | 3836 | CA | ALA | L | 84 | 11.981 | 0.793 | 4.616 | 1.00 | 15.99 | L | C |
| ATOM | 3837 | CB | ALA | L | 84 | 12.370 | 2.246 | 4.838 | 1.00 | 18.07 | L | C |
| ATOM | 3838 | C | ALA | L | 84 | 12.056 | 0.507 | 3.142 | 1.00 | 14.77 | L | C |
| ATOM | 3839 | O | ALA | L | 84 | 12.691 | −0.449 | 2.711 | 1.00 | 14.17 | L | O |
| ATOM | 3840 | N | THR | L | 85 | 11.458 | 1.402 | 2.369 | 1.00 | 13.92 | L | N |
| ATOM | 3841 | CA | THR | L | 85 | 11.498 | 1.303 | 0.922 | 1.00 | 22.94 | L | C |
| ATOM | 3842 | CB | THR | L | 85 | 10.075 | 1.310 | 0.319 | 1.00 | 22.12 | L | C |
| ATOM | 3843 | OG1 | THR | L | 85 | 9.390 | 0.117 | 0.727 | 1.00 | 21.79 | L | O |
| ATOM | 3844 | CG2 | THR | L | 85 | 10.145 | 1.355 | −1.194 | 1.00 | 24.71 | L | C |
| ATOM | 3845 | C | THR | L | 85 | 12.374 | 2.438 | 0.332 | 1.00 | 24.00 | L | C |
| ATOM | 3846 | O | THR | L | 85 | 12.334 | 3.586 | 0.803 | 1.00 | 18.14 | L | O |
| ATOM | 3847 | N | TYR | L | 86 | 13.189 | 2.081 | −0.664 | 1.00 | 19.58 | L | N |
| ATOM | 3848 | CA | TYR | L | 86 | 14.099 | 3.011 | −1.325 | 1.00 | 17.45 | L | C |
| ATOM | 3849 | CB | TYR | L | 86 | 15.550 | 2.542 | −1.120 | 1.00 | 17.77 | L | C |
| ATOM | 3850 | CG | TYR | L | 86 | 15.967 | 2.585 | 0.340 | 1.00 | 18.60 | L | C |
| ATOM | 3851 | CD1 | TYR | L | 86 | 16.428 | 3.773 | 0.921 | 1.00 | 22.39 | L | C |
| ATOM | 3852 | CE1 | TYR | L | 86 | 16.755 | 3.842 | 2.283 | 1.00 | 17.00 | L | C |
| ATOM | 3853 | CD2 | TYR | L | 86 | 15.845 | 1.464 | 1.157 | 1.00 | 18.50 | L | C |
| ATOM | 3854 | CE2 | TYR | L | 86 | 16.169 | 1.519 | 2.515 | 1.00 | 22.99 | L | C |
| ATOM | 3855 | CZ | TYR | L | 86 | 16.626 | 2.718 | 3.068 | 1.00 | 24.08 | L | C |
| ATOM | 3856 | OH | TYR | L | 86 | 16.951 | 2.782 | 4.401 | 1.00 | 20.89 | L | O |
| ATOM | 3857 | C | TYR | L | 86 | 13.777 | 3.201 | −2.811 | 1.00 | 18.94 | L | C |
| ATOM | 3858 | O | TYR | L | 86 | 13.484 | 2.247 | −3.509 | 1.00 | 21.49 | L | O |
| ATOM | 3859 | N | TYR | L | 87 | 13.838 | 4.444 | −3.275 | 1.00 | 18.61 | L | N |
| ATOM | 3860 | CA | TYR | L | 87 | 13.543 | 4.789 | −4.656 | 1.00 | 18.36 | L | C |
| ATOM | 3861 | CB | TYR | L | 87 | 12.246 | 5.603 | −4.721 | 1.00 | 18.08 | L | C |
| ATOM | 3862 | CG | TYR | L | 87 | 10.981 | 4.888 | −4.359 | 1.00 | 17.76 | L | C |
| ATOM | 3863 | CD1 | TYR | L | 87 | 10.300 | 4.117 | −5.304 | 1.00 | 20.66 | L | C |
| ATOM | 3864 | CE1 | TYR | L | 87 | 9.108 | 3.479 | −4.991 | 1.00 | 18.21 | L | C |
| ATOM | 3865 | CD2 | TYR | L | 87 | 10.437 | 5.009 | −3.088 | 1.00 | 18.27 | L | C |
| ATOM | 3866 | CE2 | TYR | L | 87 | 9.250 | 4.387 | −2.759 | 1.00 | 19.71 | L | C |
| ATOM | 3867 | CZ | TYR | L | 87 | 8.586 | 3.617 | −3.710 | 1.00 | 24.90 | L | C |
| ATOM | 3868 | OH | TYR | L | 87 | 7.420 | 2.965 | −3.367 | 1.00 | 25.57 | L | O |
| ATOM | 3869 | C | TYR | L | 87 | 14.600 | 5.687 | −5.293 | 1.00 | 23.23 | L | C |
| ATOM | 3870 | O | TYR | L | 87 | 15.134 | 6.587 | −4.637 | 1.00 | 24.35 | L | O |
| ATOM | 3871 | N | CYS | L | 88 | 14.890 | 5.475 | −6.575 | 1.00 | 23.62 | L | N |
| ATOM | 3872 | CA | CYS | L | 88 | 15.797 | 6.395 | −7.257 | 1.00 | 21.45 | L | C |
| ATOM | 3873 | C | CYS | L | 88 | 14.904 | 7.348 | −8.044 | 1.00 | 18.58 | L | C |
| ATOM | 3874 | O | CYS | L | 88 | 13.756 | 7.049 | −8.316 | 1.00 | 20.32 | L | O |
| ATOM | 3875 | CB | CYS | L | 88 | 16.798 | 5.705 | −8.177 | 1.00 | 19.98 | L | C |
| ATOM | 3876 | SG | CYS | L | 88 | 16.179 | 4.413 | −9.285 | 1.00 | 35.23 | L | S |
| ATOM | 3877 | N | LEU | L | 89 | 15.389 | 8.549 | −8.289 | 1.00 | 23.41 | L | N |
| ATOM | 3878 | CA | LEU | L | 89 | 14.650 | 9.535 | −9.055 | 1.00 | 25.16 | L | C |
| ATOM | 3879 | CB | LEU | L | 89 | 14.245 | 10.717 | −8.171 | 1.00 | 27.96 | L | C |
| ATOM | 3880 | CG | LEU | L | 89 | 13.416 | 11.858 | −8.777 | 1.00 | 31.22 | L | C |
| ATOM | 3881 | CD1 | LEU | L | 89 | 14.273 | 12.732 | −9.674 | 1.00 | 31.24 | L | C |
| ATOM | 3882 | CD2 | LEU | L | 89 | 12.216 | 11.323 | −9.530 | 1.00 | 29.41 | L | C |
| ATOM | 3883 | C | LEU | L | 89 | 15.617 | 9.975 | −10.140 | 1.00 | 27.22 | L | C |
| ATOM | 3884 | O | LEU | L | 89 | 16.803 | 10.213 | −9.882 | 1.00 | 27.47 | L | O |
| ATOM | 3885 | N | GLN | L | 90 | 15.119 | 10.049 | −11.363 | 1.00 | 27.57 | L | N |
| ATOM | 3886 | CA | GLN | L | 90 | 15.951 | 10.442 | −12.484 | 1.00 | 21.37 | L | C |
| ATOM | 3887 | CB | GLN | L | 90 | 15.879 | 9.351 | −13.567 | 1.00 | 23.53 | L | C |
| ATOM | 3888 | CG | GLN | L | 90 | 16.100 | 9.772 | −15.012 | 1.00 | 18.47 | L | C |
| ATOM | 3889 | CD | GLN | L | 90 | 14.923 | 10.494 | −15.602 | 1.00 | 19.33 | L | C |
| ATOM | 3890 | OE1 | GLN | L | 90 | 15.059 | 11.608 | −16.084 | 1.00 | 28.97 | L | O |
| ATOM | 3891 | NE2 | GLN | L | 90 | 13.747 | 9.893 | −15.520 | 1.00 | 25.19 | L | N |
| ATOM | 3892 | C | GLN | L | 90 | 15.478 | 11.772 | −13.005 | 1.00 | 20.94 | L | C |
| ATOM | 3893 | O | GLN | L | 90 | 14.280 | 11.968 | −13.178 | 1.00 | 23.65 | L | O |
| ATOM | 3894 | N | HIS | L | 91 | 16.400 | 12.719 | −13.166 | 1.00 | 24.19 | L | N |
| ATOM | 3895 | CA | HIS | L | 91 | 16.032 | 14.014 | −13.732 | 1.00 | 23.69 | L | C |
| ATOM | 3896 | CB | HIS | L | 91 | 15.960 | 15.137 | −12.673 | 1.00 | 25.56 | L | C |
| ATOM | 3897 | CG | HIS | L | 91 | 17.267 | 15.464 | −12.022 | 1.00 | 27.88 | L | C |
| ATOM | 3898 | CD2 | HIS | L | 91 | 17.902 | 14.907 | −10.965 | 1.00 | 24.13 | L | C |
| ATOM | 3899 | ND1 | HIS | L | 91 | 18.058 | 16.514 | −12.436 | 1.00 | 25.86 | L | N |
| ATOM | 3900 | CE1 | HIS | L | 91 | 19.124 | 16.593 | −11.660 | 1.00 | 23.95 | L | C |
| ATOM | 3901 | NE2 | HIS | L | 91 | 19.054 | 15.629 | −10.760 | 1.00 | 30.53 | L | N |
| ATOM | 3902 | C | HIS | L | 91 | 16.879 | 14.405 | −14.959 | 1.00 | 26.81 | L | C |
| ATOM | 3903 | O | HIS | L | 91 | 16.944 | 15.584 | −15.330 | 1.00 | 23.70 | L | O |
| ATOM | 3904 | N | GLY | L | 92 | 17.437 | 13.392 | −15.635 | 1.00 | 22.76 | L | N |
| ATOM | 3905 | CA | GLY | L | 92 | 18.239 | 13.616 | −16.832 | 1.00 | 28.83 | L | C |
| ATOM | 3906 | C | GLY | L | 92 | 17.405 | 14.009 | −18.046 | 1.00 | 29.69 | L | C |
| ATOM | 3907 | O | GLY | L | 92 | 17.943 | 14.411 | −19.081 | 1.00 | 28.34 | L | O |
| ATOM | 3908 | N | GLU | L | 93 | 16.086 | 13.928 | −17.887 | 1.00 | 25.32 | L | N |
| ATOM | 3909 | CA | GLU | L | 93 | 15.111 | 14.257 | −18.922 | 1.00 | 30.22 | L | C |
| ATOM | 3910 | CB | GLU | L | 93 | 14.967 | 13.079 | −19.905 | 1.00 | 27.55 | L | C |
| ATOM | 3911 | CG | GLU | L | 93 | 13.861 | 13.269 | −20.960 | 1.00 | 32.77 | L | C |
| ATOM | 3912 | CD | GLU | L | 93 | 13.569 | 12.003 | −21.780 | 1.00 | 41.99 | L | C |
| ATOM | 3913 | OE1 | GLU | L | 93 | 13.918 | 11.966 | −22.983 | 1.00 | 42.06 | L | O |
| ATOM | 3914 | OE2 | GLU | L | 93 | 12.985 | 11.044 | −21.228 | 1.00 | 39.47 | L | O |
| ATOM | 3915 | C | GLU | L | 93 | 13.747 | 14.494 | −18.274 | 1.00 | 27.84 | L | C |
| ATOM | 3916 | O | GLU | L | 93 | 13.426 | 13.902 | −17.245 | 1.00 | 33.52 | L | O |
| ATOM | 3917 | N | SER | L | 94 | 12.960 | 15.380 | −18.863 | 1.00 | 28.68 | L | N |
| ATOM | 3918 | CA | SER | L | 94 | 11.608 | 15.626 | −18.377 | 1.00 | 25.88 | L | C |
| ATOM | 3919 | CB | SER | L | 94 | 11.193 | 17.058 | −18.648 | 1.00 | 21.24 | L | C |
| ATOM | 3920 | OG | SER | L | 94 | 9.842 | 17.248 | −18.286 | 1.00 | 33.40 | L | O |
| ATOM | 3921 | C | SER | L | 94 | 10.745 | 14.674 | −19.199 | 1.00 | 31.34 | L | C |
| ATOM | 3922 | O | SER | L | 94 | 10.868 | 14.636 | −20.419 | 1.00 | 30.96 | L | O |
| ATOM | 3923 | N | PRO | L | 95 | 9.798 | 13.961 | −18.562 | 1.00 | 34.47 | L | N |
| ATOM | 3924 | CD | PRO | L | 95 | 8.958 | 13.019 | −19.322 | 1.00 | 24.57 | L | C |
| ATOM | 3925 | CA | PRO | L | 95 | 9.461 | 13.918 | −17.130 | 1.00 | 30.06 | L | C |
| ATOM | 3926 | CB | PRO | L | 95 | 8.092 | 13.247 | −17.143 | 1.00 | 31.54 | L | C |
| ATOM | 3927 | CG | PRO | L | 95 | 8.287 | 12.220 | −18.220 | 1.00 | 33.31 | L | C |
| ATOM | 3928 | C | PRO | L | 95 | 10.416 | 13.128 | −16.245 | 1.00 | 25.86 | L | C |
| ATOM | 3929 | O | PRO | L | 95 | 11.028 | 12.154 | −16.680 | 1.00 | 26.34 | L | O |
| ATOM | 3930 | N | TRP | L | 96 | 10.514 | 13.534 | −14.985 | 1.00 | 23.32 | L | N |
| ATOM | 3931 | CA | TRP | L | 96 | 11.369 | 12.833 | −14.040 | 1.00 | 21.44 | L | C |
| ATOM | 3932 | CB | TRP | L | 96 | 11.583 | 13.665 | −12.771 | 1.00 | 24.99 | L | C |
| ATOM | 3933 | CG | TRP | L | 96 | 12.147 | 15.028 | −13.034 | 1.00 | 25.05 | L | C |
| ATOM | 3934 | CD2 | TRP | L | 96 | 12.555 | 15.994 | −12.059 | 1.00 | 22.26 | L | C |
| ATOM | 3935 | CE2 | TRP | L | 96 | 13.069 | 17.102 | −12.759 | 1.00 | 23.29 | L | C |
| ATOM | 3936 | CE3 | TRP | L | 96 | 12.545 | 16.026 | −10.661 | 1.00 | 26.48 | L | C |
| ATOM | 3937 | CD1 | TRP | L | 96 | 12.412 | 15.579 | −14.250 | 1.00 | 24.60 | L | C |
| ATOM | 3938 | NE1 | TRP | L | 96 | 12.968 | 16.823 | −14.096 | 1.00 | 31.24 | L | N |
| ATOM | 3939 | CZ2 | TRP | L | 96 | 13.575 | 18.235 | −12.112 | 1.00 | 26.01 | L | C |
| ATOM | 3940 | CZ3 | TRP | L | 96 | 13.054 | 17.154 | −10.013 | 1.00 | 27.72 | L | C |
| ATOM | 3941 | CH2 | TRP | L | 96 | 13.561 | 18.241 | −10.741 | 1.00 | 26.93 | L | C |
| ATOM | 3942 | C | TRP | L | 96 | 10.628 | 11.554 | −13.713 | 1.00 | 20.35 | L | C |
| ATOM | 3943 | O | TRP | L | 96 | 9.392 | 11.520 | −13.700 | 1.00 | 20.01 | L | O |
| ATOM | 3944 | N | THR | L | 97 | 11.378 | 10.489 | −13.492 | 1.00 | 22.34 | L | N |
| ATOM | 3945 | CA | THR | L | 97 | 10.767 | 9.217 | −13.179 | 1.00 | 21.18 | L | C |
| ATOM | 3946 | CB | THR | L | 97 | 10.702 | 8.319 | −14.426 | 1.00 | 23.12 | L | C |
| ATOM | 3947 | OG1 | THR | L | 97 | 12.016 | 8.177 | −14.964 | 1.00 | 25.07 | L | O |
| ATOM | 3948 | CG2 | THR | L | 97 | 9.788 | 8.926 | −15.510 | 1.00 | 21.74 | L | C |
| ATOM | 3949 | C | THR | L | 97 | 11.525 | 8.492 | −12.065 | 1.00 | 27.81 | L | C |
| ATOM | 3950 | O | THR | L | 97 | 12.753 | 8.616 | −11.936 | 1.00 | 23.03 | L | O |
| ATOM | 3951 | N | PHE | L | 98 | 10.766 | 7.724 | −11.284 | 1.00 | 25.28 | L | N |
| ATOM | 3952 | CA | PHE | L | 98 | 11.278 | 6.952 | −10.163 | 1.00 | 24.40 | L | C |
| ATOM | 3953 | CB | PHE | L | 98 | 10.278 | 6.990 | −8.992 | 1.00 | 23.23 | L | C |
| ATOM | 3954 | CG | PHE | L | 98 | 10.020 | 8.348 | −8.443 | 1.00 | 21.42 | L | C |
| ATOM | 3955 | CD1 | PHE | L | 98 | 9.099 | 9.199 | −9.055 | 1.00 | 12.01 | L | C |
| ATOM | 3956 | CD2 | PHE | L | 98 | 10.668 | 8.766 | −7.281 | 1.00 | 17.32 | L | C |
| ATOM | 3957 | CE1 | PHE | L | 98 | 8.822 | 10.448 | −8.513 | 1.00 | 21.25 | L | C |
| ATOM | 3958 | CE2 | PHE | L | 98 | 10.398 | 10.029 | −6.724 | 1.00 | 16.64 | L | C |
| ATOM | 3959 | CZ | PHE | L | 98 | 9.483 | 10.868 | −7.326 | 1.00 | 16.97 | L | C |
| ATOM | 3960 | C | PHE | L | 98 | 11.421 | 5.487 | −10.523 | 1.00 | 22.50 | L | C |
| ATOM | 3961 | O | PHE | L | 98 | 10.739 | 4.993 | −11.408 | 1.00 | 24.13 | L | O |
| ATOM | 3962 | N | GLY | L | 99 | 12.269 | 4.786 | −9.778 | 1.00 | 24.86 | L | N |
| ATOM | 3963 | CA | GLY | L | 99 | 12.416 | 3.357 | −9.969 | 1.00 | 23.45 | L | C |
| ATOM | 3964 | C | GLY | L | 99 | 11.190 | 2.717 | −9.323 | 1.00 | 28.02 | L | C |
| ATOM | 3965 | O | GLY | L | 99 | 10.403 | 3.400 | −8.656 | 1.00 | 24.85 | L | O |
| ATOM | 3966 | N | GLN | L | 100 | 11.025 | 1.410 | −9.482 | 1.00 | 27.53 | L | N |
| ATOM | 3967 | CA | GLN | L | 100 | 9.860 | 0.747 | −8.918 | 1.00 | 27.19 | L | C |
| ATOM | 3968 | CB | GLN | L | 100 | 9.653 | −0.635 | −9.536 | 1.00 | 30.11 | L | C |
| ATOM | 3969 | CG | GLN | L | 100 | 9.339 | −0.607 | −11.022 | 1.00 | 50.84 | L | C |
| ATOM | 3970 | CD | GLN | L | 100 | 10.585 | −0.703 | −11.914 | 1.00 | 57.27 | L | C |
| ATOM | 3971 | OE1 | GLN | L | 100 | 11.062 | −1.804 | −12.228 | 1.00 | 60.67 | L | O |
| ATOM | 3972 | NE2 | GLN | L | 100 | 11.094 | 0.447 | −12.348 | 1.00 | 57.70 | L | N |
| ATOM | 3973 | C | GLN | L | 100 | 9.951 | 0.621 | −7.420 | 1.00 | 27.25 | L | C |
| ATOM | 3974 | O | GLN | L | 100 | 8.948 | 0.398 | −6.757 | 1.00 | 29.37 | L | O |
| ATOM | 3975 | N | GLY | L | 101 | 11.163 | 0.740 | −6.888 | 1.00 | 29.57 | L | N |
| ATOM | 3976 | CA | GLY | L | 101 | 11.352 | 0.656 | −5.456 | 1.00 | 20.97 | L | C |
| ATOM | 3977 | C | GLY | L | 101 | 12.068 | −0.607 | −5.043 | 1.00 | 24.72 | L | C |
| ATOM | 3978 | O | GLY | L | 101 | 12.190 | −1.543 | −5.829 | 1.00 | 25.42 | L | O |
| ATOM | 3979 | N | THR | L | 102 | 12.624 | −0.581 | −3.837 | 1.00 | 24.59 | L | N |
| ATOM | 3980 | CA | THR | L | 102 | 13.323 | −1.714 | −3.247 | 1.00 | 20.35 | L | C |
| ATOM | 3981 | CB | THR | L | 102 | 14.866 | −1.607 | −3.364 | 1.00 | 22.47 | L | C |
| ATOM | 3982 | OG1 | THR | L | 102 | 15.259 | −1.640 | −4.741 | 1.00 | 25.78 | L | O |
| ATOM | 3983 | CG2 | THR | L | 102 | 15.540 | −2.760 | −2.624 | 1.00 | 11.04 | L | C |
| ATOM | 3984 | C | THR | L | 102 | 12.981 | −1.654 | −1.777 | 1.00 | 23.56 | L | C |
| ATOM | 3985 | O | THR | L | 102 | 13.292 | −0.667 | −1.098 | 1.00 | 22.89 | L | O |
| ATOM | 3986 | N | LYS | L | 103 | 12.250 | −2.658 | −1.319 | 1.00 | 24.95 | L | N |
| ATOM | 3987 | CA | LYS | L | 103 | 11.870 | −2.743 | 0.076 | 1.00 | 24.58 | L | C |
| ATOM | 3988 | CB | LYS | L | 103 | 10.504 | −3.393 | 0.222 | 1.00 | 28.56 | L | C |
| ATOM | 3989 | CG | LYS | L | 103 | 10.067 | −3.575 | 1.647 | 1.00 | 32.24 | L | C |
| ATOM | 3990 | CD | LYS | L | 103 | 8.722 | −4.270 | 1.691 | 1.00 | 37.30 | L | C |
| ATOM | 3991 | CE | LYS | L | 103 | 8.235 | −4.475 | 3.110 | 1.00 | 44.31 | L | C |
| ATOM | 3992 | NZ | LYS | L | 103 | 6.850 | −5.054 | 3.142 | 1.00 | 53.72 | L | N |
| ATOM | 3993 | C | LYS | L | 103 | 12.915 | −3.581 | 0.782 | 1.00 | 24.04 | L | C |
| ATOM | 3994 | O | LYS | L | 103 | 13.249 | −4.690 | 0.331 | 1.00 | 20.49 | L | O |
| ATOM | 3995 | N | VAL | L | 104 | 13.488 | −3.008 | 1.837 | 1.00 | 18.11 | L | N |
| ATOM | 3996 | CA | VAL | L | 104 | 14.490 | −3.699 | 2.637 | 1.00 | 17.98 | L | C |
| ATOM | 3997 | CB | VAL | L | 104 | 15.652 | −2.754 | 2.982 | 1.00 | 21.07 | L | C |
| ATOM | 3998 | CG1 | VAL | L | 104 | 16.683 | −3.465 | 3.844 | 1.00 | 14.17 | L | C |
| ATOM | 3999 | CG2 | VAL | L | 104 | 16.274 | −2.215 | 1.680 | 1.00 | 14.28 | L | C |
| ATOM | 4000 | C | VAL | L | 104 | 13.752 | −4.183 | 3.885 | 1.00 | 23.64 | L | C |
| ATOM | 4001 | O | VAL | L | 104 | 13.218 | −3.391 | 4.672 | 1.00 | 24.04 | L | O |
| ATOM | 4002 | N | GLU | L | 105 | 13.661 | −5.498 | 4.014 | 1.00 | 23.08 | L | N |
| ATOM | 4003 | CA | GLU | L | 105 | 12.950 | −6.107 | 5.121 | 1.00 | 21.40 | L | C |
| ATOM | 4004 | CB | GLU | L | 105 | 11.754 | −6.902 | 4.586 | 1.00 | 25.03 | L | C |
| ATOM | 4005 | CG | GLU | L | 105 | 12.116 | −7.927 | 3.521 | 1.00 | 32.86 | L | C |
| ATOM | 4006 | CD | GLU | L | 105 | 12.183 | −9.326 | 4.075 | 1.00 | 35.40 | L | C |
| ATOM | 4007 | OE1 | GLU | L | 105 | 11.114 | −9.893 | 4.371 | 1.00 | 43.34 | L | O |
| ATOM | 4008 | OE2 | GLU | L | 105 | 13.298 | −9.865 | 4.227 | 1.00 | 40.08 | L | O |
| ATOM | 4009 | C | GLU | L | 105 | 13.860 | −6.979 | 5.949 | 1.00 | 18.07 | L | C |
| ATOM | 4010 | O | GLU | L | 105 | 14.930 | −7.377 | 5.494 | 1.00 | 25.62 | L | O |
| ATOM | 4011 | N | ILE | L | 106 | 13.406 | −7.303 | 7.155 | 1.00 | 22.75 | L | N |
| ATOM | 4012 | CA | ILE | L | 106 | 14.186 | −8.088 | 8.095 | 1.00 | 23.54 | L | C |
| ATOM | 4013 | CB | ILE | L | 106 | 13.666 | −7.925 | 9.526 | 1.00 | 22.22 | L | C |
| ATOM | 4014 | CG2 | ILE | L | 106 | 14.717 | −8.484 | 10.526 | 1.00 | 18.70 | L | C |
| ATOM | 4015 | CG1 | ILE | L | 106 | 13.426 | −6.444 | 9.821 | 1.00 | 25.97 | L | C |
| ATOM | 4016 | CD1 | ILE | L | 106 | 12.811 | −6.173 | 11.159 | 1.00 | 24.51 | L | C |
| ATOM | 4017 | C | ILE | L | 106 | 14.341 | −9.574 | 7.816 | 1.00 | 29.68 | L | C |
| ATOM | 4018 | O | ILE | L | 106 | 13.357 | −10.295 | 7.702 | 1.00 | 32.83 | L | O |
| ATOM | 4019 | N | LYS | L | 107 | 15.590 | −10.034 | 7.749 | 1.00 | 31.76 | L | N |
| ATOM | 4020 | CA | LYS | L | 107 | 15.859 | −11.445 | 7.529 | 1.00 | 29.75 | L | C |
| ATOM | 4021 | CB | LYS | L | 107 | 17.262 | −11.686 | 6.945 | 1.00 | 37.08 | L | C |
| ATOM | 4022 | CG | LYS | L | 107 | 17.562 | −13.182 | 6.661 | 1.00 | 42.63 | L | C |
| ATOM | 4023 | CD | LYS | L | 107 | 18.640 | −13.407 | 5.579 | 1.00 | 52.33 | L | C |
| ATOM | 4024 | CE | LYS | L | 107 | 18.738 | −14.903 | 5.191 | 1.00 | 54.76 | L | C |
| ATOM | 4025 | NZ | LYS | L | 107 | 19.714 | −15.205 | 4.093 | 1.00 | 51.38 | L | N |
| ATOM | 4026 | C | LYS | L | 107 | 15.729 | −12.196 | 8.846 | 1.00 | 33.31 | L | C |
| ATOM | 4027 | O | LYS | L | 107 | 16.116 | −11.709 | 9.907 | 1.00 | 29.91 | L | O |
| ATOM | 4028 | N | ARG | L | 108 | 15.169 | −13.392 | 8.761 | 1.00 | 28.20 | L | N |
| ATOM | 4029 | CA | ARG | L | 108 | 14.990 | −14.248 | 9.916 | 1.00 | 23.11 | L | C |
| ATOM | 4030 | CB | ARG | L | 108 | 13.683 | −13.902 | 10.660 | 1.00 | 29.93 | L | C |
| ATOM | 4031 | CG | ARG | L | 108 | 13.816 | −12.803 | 11.706 | 0.00 | 28.29 | L | C |
| ATOM | 4032 | CD | ARG | L | 108 | 14.760 | −13.213 | 12.831 | 0.00 | 30.29 | L | C |
| ATOM | 4033 | NE | ARG | L | 108 | 14.285 | −14.394 | 13.550 | 0.00 | 30.51 | L | N |
| ATOM | 4034 | CZ | ARG | L | 108 | 14.960 | −15.536 | 13.650 | 0.00 | 31.25 | L | C |
| ATOM | 4035 | NH1 | ARG | L | 108 | 16.150 | −15.665 | 13.078 | 0.00 | 31.10 | L | N |
| ATOM | 4036 | NH2 | ARG | L | 108 | 14.440 | −16.556 | 14.320 | 0.00 | 31.10 | L | N |
| ATOM | 4037 | C | ARG | L | 108 | 14.944 | −15.670 | 9.366 | 1.00 | 21.73 | L | C |
| ATOM | 4038 | O | ARG | L | 108 | 14.983 | −15.861 | 8.153 | 1.00 | 15.31 | L | O |
| ATOM | 4039 | N | THR | L | 109 | 14.871 | −16.664 | 10.244 | 1.00 | 18.25 | L | N |
| ATOM | 4040 | CA | THR | L | 109 | 14.827 | −18.051 | 9.784 | 1.00 | 25.89 | L | C |
| ATOM | 4041 | CB | THR | L | 109 | 15.088 | −19.051 | 10.942 | 1.00 | 21.81 | L | C |
| ATOM | 4042 | OG1 | THR | L | 109 | 14.196 | −18.768 | 12.026 | 1.00 | 35.78 | L | O |
| ATOM | 4043 | CG2 | THR | L | 109 | 16.527 | −18.960 | 11.445 | 1.00 | 21.38 | L | C |
| ATOM | 4044 | C | THR | L | 109 | 13.498 | −18.399 | 9.086 | 1.00 | 26.65 | L | C |
| ATOM | 4045 | O | THR | L | 109 | 12.469 | −17.744 | 9.305 | 1.00 | 28.20 | L | O |
| ATOM | 4046 | N | VAL | L | 110 | 13.559 | −19.400 | 8.209 | 1.00 | 25.30 | L | N |
| ATOM | 4047 | CA | VAL | L | 110 | 12.407 | −19.893 | 7.469 | 1.00 | 24.93 | L | C |
| ATOM | 4048 | CB | VAL | L | 110 | 12.819 | −21.038 | 6.531 | 1.00 | 27.40 | L | C |
| ATOM | 4049 | CG1 | VAL | L | 110 | 11.604 | −21.751 | 5.995 | 1.00 | 23.45 | L | C |
| ATOM | 4050 | CG2 | VAL | L | 110 | 13.643 | −20.480 | 5.378 | 1.00 | 24.94 | L | C |
| ATOM | 4051 | C | VAL | L | 110 | 11.316 | −20.383 | 8.419 | 1.00 | 28.87 | L | C |
| ATOM | 4052 | O | VAL | L | 110 | 11.560 | −21.225 | 9.292 | 1.00 | 37.01 | L | O |
| ATOM | 4053 | N | ALA | L | 111 | 10.118 | −19.829 | 8.260 | 1.00 | 24.76 | L | N |
| ATOM | 4054 | CA | ALA | L | 111 | 8.986 | −20.182 | 9.095 | 1.00 | 21.43 | L | C |
| ATOM | 4055 | CB | ALA | L | 111 | 8.573 | −19.000 | 9.941 | 1.00 | 18.93 | L | C |
| ATOM | 4056 | C | ALA | L | 111 | 7.834 | −20.597 | 8.221 | 1.00 | 23.34 | L | C |
| ATOM | 4057 | O | ALA | L | 111 | 7.346 | −19.803 | 7.420 | 1.00 | 25.66 | L | O |
| ATOM | 4058 | N | ALA | L | 112 | 7.415 | −21.848 | 8.369 | 1.00 | 25.12 | L | N |
| ATOM | 4059 | CA | ALA | L | 112 | 6.298 | −22.397 | 7.602 | 1.00 | 30.48 | L | C |
| ATOM | 4060 | CB | ALA | L | 112 | 6.290 | −23.914 | 7.692 | 1.00 | 27.39 | L | C |
| ATOM | 4061 | C | ALA | L | 112 | 4.949 | −21.846 | 8.063 | 1.00 | 31.68 | L | C |
| ATOM | 4062 | O | ALA | L | 112 | 4.678 | −21.730 | 9.269 | 1.00 | 33.01 | L | O |
| ATOM | 4063 | N | PRO | L | 113 | 4.073 | −21.518 | 7.103 | 1.00 | 30.88 | L | N |
| ATOM | 4064 | CD | PRO | L | 113 | 4.284 | −21.524 | 5.641 | 1.00 | 26.98 | L | C |
| ATOM | 4065 | CA | PRO | L | 113 | 2.753 | −20.984 | 7.453 | 1.00 | 29.96 | L | C |
| ATOM | 4066 | CB | PRO | L | 113 | 2.215 | −20.489 | 6.107 | 1.00 | 23.35 | L | C |
| ATOM | 4067 | CG | PRO | L | 113 | 2.880 | −21.408 | 5.103 | 1.00 | 24.64 | L | C |
| ATOM | 4068 | C | PRO | L | 113 | 1.829 | −22.037 | 8.026 | 1.00 | 33.26 | L | C |
| ATOM | 4069 | O | PRO | L | 113 | 1.910 | −23.210 | 7.649 | 1.00 | 36.07 | L | O |
| ATOM | 4070 | N | SER | L | 114 | 1.035 | −21.655 | 9.021 | 1.00 | 32.90 | L | N |
| ATOM | 4071 | CA | SER | L | 114 | 0.044 | −22.588 | 9.542 | 1.00 | 35.20 | L | C |
| ATOM | 4072 | CB | SER | L | 114 | −0.202 | −22.401 | 11.052 | 1.00 | 31.73 | L | C |
| ATOM | 4073 | OG | SER | L | 114 | −0.577 | −21.080 | 11.396 | 1.00 | 38.73 | L | O |
| ATOM | 4074 | C | SER | L | 114 | −1.162 | −22.195 | 8.661 | 1.00 | 38.79 | L | C |
| ATOM | 4075 | O | SER | L | 114 | −1.485 | −21.011 | 8.544 | 1.00 | 37.34 | L | O |
| ATOM | 4076 | N | VAL | L | 115 | −1.707 | −23.164 | 7.921 | 1.00 | 40.84 | L | N |
| ATOM | 4077 | CA | VAL | L | 115 | −2.824 | −22.919 | 7.001 | 1.00 | 37.58 | L | C |
| ATOM | 4078 | CB | VAL | L | 115 | −2.620 | −23.674 | 5.684 | 1.00 | 32.23 | L | C |
| ATOM | 4079 | CG1 | VAL | L | 115 | −3.551 | −23.125 | 4.621 | 1.00 | 34.46 | L | C |
| ATOM | 4080 | CG2 | VAL | L | 115 | −1.181 | −23.578 | 5.244 | 1.00 | 30.20 | L | C |
| ATOM | 4081 | C | VAL | L | 115 | −4.206 | −23.292 | 7.541 | 1.00 | 39.50 | L | C |
| ATOM | 4082 | O | VAL | L | 115 | −4.381 | −24.353 | 8.130 | 1.00 | 43.94 | L | O |
| ATOM | 4083 | N | PHE | L | 116 | −5.190 | −22.434 | 7.271 | 1.00 | 39.31 | L | N |
| ATOM | 4084 | CA | PHE | L | 116 | −6.571 | −22.628 | 7.717 | 1.00 | 38.14 | L | C |
| ATOM | 4085 | CB | PHE | L | 116 | −6.860 | −21.717 | 8.906 | 1.00 | 34.77 | L | C |
| ATOM | 4086 | CG | PHE | L | 116 | −5.941 | −21.903 | 10.066 | 1.00 | 39.00 | L | C |
| ATOM | 4087 | CD1 | PHE | L | 116 | −4.696 | −21.279 | 10.094 | 1.00 | 39.39 | L | C |
| ATOM | 4088 | CD2 | PHE | L | 116 | −6.340 | −22.651 | 11.167 | 1.00 | 39.33 | L | C |
| ATOM | 4089 | CE1 | PHE | L | 116 | −3.863 | −21.391 | 11.203 | 1.00 | 40.11 | L | C |
| ATOM | 4090 | CE2 | PHE | L | 116 | −5.512 | −22.769 | 12.283 | 1.00 | 44.30 | L | C |
| ATOM | 4091 | CZ | PHE | L | 116 | −4.266 | −22.133 | 12.300 | 1.00 | 40.48 | L | C |
| ATOM | 4092 | C | PHE | L | 116 | −7.558 | −22.238 | 6.608 | 1.00 | 35.36 | L | C |
| ATOM | 4093 | O | PHE | L | 116 | −7.347 | −21.238 | 5.928 | 1.00 | 36.64 | L | O |
| ATOM | 4094 | N | ILE | L | 117 | −8.652 | −22.983 | 6.459 | 1.00 | 31.75 | L | N |
| ATOM | 4095 | CA | ILE | L | 117 | −9.658 | −22.649 | 5.442 | 1.00 | 36.27 | L | C |
| ATOM | 4096 | CB | ILE | L | 117 | −9.736 | −23.725 | 4.306 | 1.00 | 36.46 | L | C |
| ATOM | 4097 | CG2 | ILE | L | 117 | −10.348 | −25.010 | 4.814 | 1.00 | 35.51 | L | C |
| ATOM | 4098 | CG1 | ILE | L | 117 | −10.575 | −23.206 | 3.141 | 1.00 | 35.51 | L | C |
| ATOM | 4099 | CD1 | ILE | L | 117 | −10.644 | −24.157 | 1.983 | 1.00 | 28.86 | L | C |
| ATOM | 4100 | C | ILE | L | 117 | −11.030 | −22.411 | 6.098 | 1.00 | 33.48 | L | C |
| ATOM | 4101 | O | ILE | L | 117 | −11.402 | −23.106 | 7.041 | 1.00 | 33.88 | L | O |
| ATOM | 4102 | N | PHE | L | 118 | −11.732 | −21.370 | 5.655 | 1.00 | 32.97 | L | N |
| ATOM | 4103 | CA | PHE | L | 118 | −13.044 | −21.025 | 6.210 | 1.00 | 29.25 | L | C |
| ATOM | 4104 | CB | PHE | L | 118 | −13.053 | −19.605 | 6.770 | 1.00 | 25.34 | L | C |
| ATOM | 4105 | CG | PHE | L | 118 | −12.079 | −19.379 | 7.872 | 1.00 | 32.04 | L | C |
| ATOM | 4106 | CD1 | PHE | L | 118 | −12.414 | −19.692 | 9.188 | 1.00 | 33.78 | L | C |
| ATOM | 4107 | CD2 | PHE | L | 118 | −10.818 | −18.853 | 7.604 | 1.00 | 28.30 | L | C |
| ATOM | 4108 | CE1 | PHE | L | 118 | −11.498 | −19.482 | 10.236 | 1.00 | 31.17 | L | C |
| ATOM | 4109 | CE2 | PHE | L | 118 | −9.902 | −18.641 | 8.630 | 1.00 | 28.94 | L | C |
| ATOM | 4110 | CZ | PHE | L | 118 | −10.244 | −18.959 | 9.956 | 1.00 | 30.76 | L | C |
| ATOM | 4111 | C | PHE | L | 118 | −14.112 | −21.074 | 5.132 | 1.00 | 36.99 | L | C |
| ATOM | 4112 | O | PHE | L | 118 | −13.957 | −20.476 | 4.058 | 1.00 | 37.80 | L | O |
| ATOM | 4113 | N | PRO | L | 119 | −15.209 | −21.803 | 5.390 | 1.00 | 40.02 | L | N |
| ATOM | 4114 | CD | PRO | L | 119 | −15.418 | −22.754 | 6.493 | 1.00 | 38.43 | L | C |
| ATOM | 4115 | CA | PRO | L | 119 | −16.297 | −21.896 | 4.407 | 1.00 | 37.22 | L | C |
| ATOM | 4116 | CB | PRO | L | 119 | −17.069 | −23.146 | 4.855 | 1.00 | 35.18 | L | C |
| ATOM | 4117 | CG | PRO | L | 119 | −16.106 | −23.878 | 5.774 | 1.00 | 44.30 | L | C |
| ATOM | 4118 | C | PRO | L | 119 | −17.185 | −20.668 | 4.566 | 1.00 | 37.73 | L | C |
| ATOM | 4119 | O | PRO | L | 119 | −17.071 | −19.935 | 5.558 | 1.00 | 36.96 | L | O |
| ATOM | 4120 | N | PRO | L | 120 | −18.048 | −20.396 | 3.577 | 1.00 | 37.12 | L | N |
| ATOM | 4121 | CD | PRO | L | 120 | −18.170 | −20.991 | 2.237 | 1.00 | 38.99 | L | C |
| ATOM | 4122 | CA | PRO | L | 120 | −18.921 | −19.230 | 3.717 | 1.00 | 34.28 | L | C |
| ATOM | 4123 | CB | PRO | L | 120 | −19.615 | −19.149 | 2.362 | 1.00 | 36.26 | L | C |
| ATOM | 4124 | CG | PRO | L | 120 | −19.547 | −20.558 | 1.840 | 1.00 | 40.75 | L | C |
| ATOM | 4125 | C | PRO | L | 120 | −19.908 | −19.474 | 4.856 | 1.00 | 35.24 | L | C |
| ATOM | 4126 | O | PRO | L | 120 | −20.294 | −20.616 | 5.127 | 1.00 | 33.80 | L | O |
| ATOM | 4127 | N | SER | L | 121 | −20.216 | −18.410 | 5.590 | 1.00 | 35.62 | L | N |
| ATOM | 4128 | CA | SER | L | 121 | −21.150 | −18.483 | 6.710 | 1.00 | 35.43 | L | C |
| ATOM | 4129 | CB | SER | L | 121 | −21.009 | −17.235 | 7.598 | 1.00 | 35.29 | L | C |
| ATOM | 4130 | OG | SER | L | 121 | −21.333 | −16.043 | 6.898 | 1.00 | 38.71 | L | O |
| ATOM | 4131 | C | SER | L | 121 | −22.573 | −18.591 | 6.173 | 1.00 | 35.86 | L | C |
| ATOM | 4132 | O | SER | L | 121 | −22.863 | −18.119 | 5.070 | 1.00 | 30.10 | L | O |
| ATOM | 4133 | N | ASP | L | 122 | −23.456 | −19.216 | 6.949 | 1.00 | 37.62 | L | N |
| ATOM | 4134 | CA | ASP | L | 122 | −24.852 | −19.379 | 6.534 | 1.00 | 39.63 | L | C |
| ATOM | 4135 | CB | ASP | L | 122 | −25.651 | −20.188 | 7.576 | 1.00 | 42.45 | L | C |
| ATOM | 4136 | CG | ASP | L | 122 | −25.301 | −21.671 | 7.563 | 1.00 | 46.25 | L | C |
| ATOM | 4137 | OD1 | ASP | L | 122 | −25.141 | −22.233 | 6.455 | 1.00 | 48.10 | L | O |
| ATOM | 4138 | OD2 | ASP | L | 122 | −25.187 | −22.279 | 8.654 | 1.00 | 49.08 | L | O |
| ATOM | 4139 | C | ASP | L | 122 | −25.502 | −18.018 | 6.297 | 1.00 | 36.82 | L | C |
| ATOM | 4140 | O | ASP | L | 122 | −26.279 | −17.848 | 5.354 | 1.00 | 36.06 | L | O |
| ATOM | 4141 | N | GLU | L | 123 | −25148 | −17.046 | 7.132 | 1.00 | 31.03 | L | N |
| ATOM | 4142 | CA | GLU | L | 123 | −25.691 | −15.705 | 6.990 | 1.00 | 37.17 | L | C |
| ATOM | 4143 | CB | GLU | L | 123 | −25.344 | −14.829 | 8.201 | 1.00 | 43.19 | L | C |
| ATOM | 4144 | CG | GLU | L | 123 | −24.210 | −15.358 | 9.078 | 1.00 | 51.48 | L | C |
| ATOM | 4145 | CD | GLU | L | 123 | −24.015 | −14.516 | 10.333 | 1.00 | 61.15 | L | C |
| ATOM | 4146 | OE1 | GLU | L | 123 | −24.146 | −13.265 | 10.236 | 1.00 | 60.00 | L | O |
| ATOM | 4147 | OE2 | GLU | L | 123 | −23.743 | −15.106 | 11.413 | 1.00 | 60.26 | L | O |
| ATOM | 4148 | C | GLU | L | 123 | −25.298 | −15.017 | 5.676 | 1.00 | 38.18 | L | C |
| ATOM | 4149 | O | GLU | L | 123 | −26.081 | −14.226 | 5.141 | 1.00 | 39.82 | L | O |
| ATOM | 4150 | N | GLN | L | 124 | −24.104 | −15.308 | 5.147 | 1.00 | 35.94 | L | N |
| ATOM | 4151 | CA | GLN | L | 124 | −23.690 | −14.697 | 3.878 | 1.00 | 34.94 | L | C |
| ATOM | 4152 | CB | GLN | L | 124 | −22.156 | −14.716 | 3.701 | 1.00 | 31.18 | L | C |
| ATOM | 4153 | CG | GLN | L | 124 | −21.691 | −14.076 | 2.387 | 1.00 | 22.46 | L | C |
| ATOM | 4154 | CD | GLN | L | 124 | −20.232 | −14.351 | 2.024 | 1.00 | 31.27 | L | C |
| ATOM | 4155 | OE1 | GLN | L | 124 | −19.583 | −15.260 | 2.563 | 1.00 | 31.73 | L | O |
| ATOM | 4156 | NE2 | GLN | L | 124 | −19.718 | −13.572 | 1.077 | 1.00 | 28.92 | L | N |
| ATOM | 4157 | C | GLN | L | 124 | −24.386 | −15.397 | 2.689 | 1.00 | 31.85 | L | C |
| ATOM | 4158 | O | GLN | L | 124 | −24.833 | −14.743 | 1.740 | 1.00 | 30.87 | L | O |
| ATOM | 4159 | N | LEU | L | 125 | −24.473 | −16.722 | 2.751 | 1.00 | 26.61 | L | N |
| ATOM | 4160 | CA | LEU | L | 125 | −25.117 | −17.492 | 1.699 | 1.00 | 32.93 | L | C |
| ATOM | 4161 | CB | LEU | L | 125 | −25.113 | −18.969 | 2.057 | 1.00 | 31.08 | L | C |
| ATOM | 4162 | CG | LEU | L | 125 | −23.748 | −19.581 | 1.774 | 1.00 | 31.01 | L | C |
| ATOM | 4163 | CD1 | LEU | L | 125 | −23.727 | −21.048 | 2.156 | 1.00 | 32.70 | L | C |
| ATOM | 4164 | CD2 | LEU | L | 125 | −23.451 | −19.400 | 0.297 | 1.00 | 29.51 | L | C |
| ATOM | 4165 | C | LEU | L | 125 | −26.534 | −16.987 | 1.492 | 1.00 | 37.46 | L | C |
| ATOM | 4166 | O | LEU | L | 125 | −26.980 | −16.838 | 0.359 | 1.00 | 41.42 | L | O |
| ATOM | 4167 | N | LYS | L | 126 | −27.189 | −16.633 | 2.598 | 1.00 | 41.12 | L | N |
| ATOM | 4168 | CA | LYS | L | 126 | −28.547 | −16.094 | 2.595 | 1.00 | 41.69 | L | C |
| ATOM | 4169 | CB | LYS | L | 126 | −28.991 | −15.765 | 4.023 | 1.00 | 46.53 | L | C |
| ATOM | 4170 | CG | LYS | L | 126 | −29.617 | −16.903 | 4.799 | 1.00 | 49.69 | L | C |
| ATOM | 4171 | CD | LYS | L | 126 | −30.430 | −16.340 | 5.964 | 1.00 | 55.74 | L | C |
| ATOM | 4172 | CE | LYS | L | 126 | −31.558 | −15.435 | 5.454 | 1.00 | 57.45 | L | C |
| ATOM | 4173 | NZ | LYS | L | 126 | −32.479 | −14.967 | 6.532 | 1.00 | 62.01 | L | N |
| ATOM | 4174 | C | LYS | L | 126 | −28.666 | −14.818 | 1.772 | 1.00 | 38.07 | L | C |
| ATOM | 4175 | O | LYS | L | 126 | −29.748 | −14.477 | 1.307 | 1.00 | 43.43 | L | O |
| ATOM | 4176 | N | SER | L | 127 | −27.569 | −14.082 | 1.641 | 1.00 | 34.37 | L | N |
| ATOM | 4177 | CA | SER | L | 127 | −27.593 | −12.840 | 0.879 | 1.00 | 31.79 | L | C |
| ATOM | 4178 | CB | SER | L | 127 | −26.668 | −11.808 | 1.516 | 1.00 | 26.93 | L | C |
| ATOM | 4179 | OG | SER | L | 127 | −25.311 | −12.219 | 1.422 | 1.00 | 33.26 | L | O |
| ATOM | 4180 | C | SER | L | 127 | −27.228 | −13.052 | −0.596 | 1.00 | 31.03 | L | C |
| ATOM | 4181 | O | SER | L | 127 | −27.101 | −12.086 | −1.357 | 1.00 | 30.54 | L | O |
| ATOM | 4182 | N | GLY | L | 128 | −27.020 | −14.309 | −0.983 | 1.00 | 24.14 | L | N |
| ATOM | 4183 | CA | GLY | L | 128 | −26.702 | −14.611 | −2.371 | 1.00 | 32.32 | L | C |
| ATOM | 4184 | C | GLY | L | 128 | −25.250 | −14.753 | −2.823 | 1.00 | 37.88 | L | C |
| ATOM | 4185 | O | GLY | L | 128 | −25.022 | −15.145 | −3.978 | 1.00 | 36.56 | L | O |
| ATOM | 4186 | N | THR | L | 129 | −24.281 | −14.440 | −1.953 | 1.00 | 28.62 | L | N |
| ATOM | 4187 | CA | THR | L | 129 | −22.866 | −14.546 | −2.307 | 1.00 | 29.63 | L | C |
| ATOM | 4188 | CB | THR | L | 129 | −22.122 | −13.196 | −2.155 | 1.00 | 24.22 | L | C |
| ATOM | 4189 | OG1 | THR | L | 129 | −22.065 | −12.821 | −0.771 | 1.00 | 32.06 | L | O |
| ATOM | 4190 | CG2 | THR | L | 129 | −22.834 | −12.114 | −2.907 | 1.00 | 27.64 | L | C |
| ATOM | 4191 | C | THR | L | 129 | −22.169 | −15.589 | −1.437 | 1.00 | 31.60 | L | C |
| ATOM | 4192 | O | THR | L | 129 | −22.700 | −16.007 | −0.411 | 1.00 | 31.26 | L | O |
| ATOM | 4193 | N | ALA | L | 130 | −20.950 | −15.952 | −1.832 | 1.00 | 32.66 | L | N |
| ATOM | 4194 | CA | ALA | L | 130 | −20.134 | −16.948 | −1.137 | 1.00 | 26.40 | L | C |
| ATOM | 4195 | CB | ALA | L | 130 | −20.275 | −18.286 | −1.840 | 1.00 | 21.34 | L | C |
| ATOM | 4196 | C | ALA | L | 130 | −18.646 | −16.575 | −1.118 | 1.00 | 30.32 | L | C |
| ATOM | 4197 | O | ALA | L | 130 | −18.011 | −16.514 | −2.168 | 1.00 | 28.31 | L | O |
| ATOM | 4198 | N | SER | L | 131 | −18.077 | −16.373 | 0.066 | 1.00 | 27.98 | L | N |
| ATOM | 4199 | CA | SER | L | 131 | −16.647 | −16.059 | 0.170 | 1.00 | 22.55 | L | C |
| ATOM | 4200 | CB | SER | L | 131 | −16.415 | −14.775 | 0.943 | 1.00 | 11.16 | L | C |
| ATOM | 4201 | OG | SER | L | 131 | −16.938 | −13.665 | 0.245 | 1.00 | 20.45 | L | O |
| ATOM | 4202 | C | SER | L | 131 | −15.957 | −17.168 | 0.931 | 1.00 | 27.84 | L | C |
| ATOM | 4203 | O | SER | L | 131 | −16.368 | −17.505 | 2.040 | 1.00 | 29.45 | L | O |
| ATOM | 4204 | N | VAL | L | 132 | −14.953 | −17.776 | 0.314 | 1.00 | 23.65 | L | N |
| ATOM | 4205 | CA | VAL | L | 132 | −14.190 | −18.819 | 0.976 | 1.00 | 28.47 | L | C |
| ATOM | 4206 | CB | VAL | L | 132 | −13.956 | −20.025 | 0.053 | 1.00 | 26.16 | L | C |
| ATOM | 4207 | CG1 | VAL | L | 132 | −13.443 | −21.180 | 0.852 | 1.00 | 27.19 | L | C |
| ATOM | 4208 | CG2 | VAL | L | 132 | −15.232 | −20.411 | −0.651 | 1.00 | 30.74 | L | C |
| ATOM | 4209 | C | VAL | L | 132 | −12.840 | −18.176 | 1.338 | 1.00 | 32.29 | L | C |
| ATOM | 4210 | O | VAL | L | 132 | −12.171 | −17.602 | 0.474 | 1.00 | 30.67 | L | O |
| ATOM | 4211 | N | VAL | L | 133 | −12.460 | −18.233 | 2.611 | 1.00 | 28.95 | L | N |
| ATOM | 4212 | CA | VAL | L | 133 | −11.201 | −17.625 | 3.043 | 1.00 | 26.43 | L | C |
| ATOM | 4213 | CB | VAL | L | 133 | −11.416 | −16.635 | 4.225 | 1.00 | 25.16 | L | C |
| ATOM | 4214 | CG1 | VAL | L | 133 | −10.087 | −16.016 | 4.658 | 1.00 | 15.96 | L | C |
| ATOM | 4215 | CG2 | VAL | L | 133 | −12.420 | −15.539 | 3.828 | 1.00 | 17.47 | L | C |
| ATOM | 4216 | C | VAL | L | 133 | −10.112 | −18.619 | 3.423 | 1.00 | 31.20 | L | C |
| ATOM | 4217 | O | VAL | L | 133 | −10.356 | −19.563 | 4.190 | 1.00 | 25.85 | L | O |
| ATOM | 4218 | N | CYS | L | 134 | −8.930 | −18.422 | 2.831 | 1.00 | 30.68 | L | N |
| ATOM | 4219 | CA | CYS | L | 134 | −7.754 | −19.248 | 3.114 | 1.00 | 34.02 | L | C |
| ATOM | 4220 | C | CYS | L | 134 | −6.732 | −18.373 | 3.858 | 1.00 | 31.77 | L | C |
| ATOM | 4221 | O | CYS | L | 134 | −6.363 | −17.292 | 3.393 | 1.00 | 27.25 | L | O |
| ATOM | 4222 | CB | CYS | L | 134 | −7.160 | −19.795 | 1.819 | 1.00 | 38.16 | L | C |
| ATOM | 4223 | SG | CYS | L | 134 | −5.741 | −20.926 | 2.028 | 1.00 | 46.01 | L | S |
| ATOM | 4224 | N | LEU | L | 135 | −6.331 | −18.818 | 5.043 | 1.00 | 27.33 | L | N |
| ATOM | 4225 | CA | LEU | L | 135 | −5.385 | −18.069 | 5.862 | 1.00 | 26.47 | L | C |
| ATOM | 4226 | CB | LEU | L | 135 | −5.970 | −17.845 | 7.266 | 1.00 | 24.98 | L | C |
| ATOM | 4227 | CG | LEU | L | 135 | −5.042 | −17.203 | 8.306 | 1.00 | 29.24 | L | C |
| ATOM | 4228 | CD1 | LEU | L | 135 | −4.864 | −15.724 | 8.008 | 1.00 | 22.82 | L | C |
| ATOM | 4229 | CD2 | LEU | L | 135 | −5.607 | −17.374 | 9.698 | 1.00 | 26.90 | L | C |
| ATOM | 4230 | C | LEU | L | 135 | −4.016 | −18.733 | 6.001 | 1.00 | 24.38 | L | C |
| ATOM | 4231 | O | LEU | L | 135 | −3.927 | −19.927 | 6.304 | 1.00 | 22.41 | L | O |
| ATOM | 4232 | N | LEU | L | 136 | −2.968 | −17.970 | 5.690 | 1.00 | 18.48 | L | N |
| ATOM | 4233 | CA | LEU | L | 136 | −1.576 | −18.419 | 5.863 | 1.00 | 22.30 | L | C |
| ATOM | 4234 | CB | LEU | L | 136 | −0.709 | −18.144 | 4.627 | 1.00 | 16.79 | L | C |
| ATOM | 4235 | CG | LEU | L | 136 | −0.833 | −19.138 | 3.464 | 1.00 | 17.31 | L | C |
| ATOM | 4236 | CD1 | LEU | L | 136 | −2.229 | −19.048 | 2.874 | 1.00 | 20.96 | L | C |
| ATOM | 4237 | CD2 | LEU | L | 136 | 0.172 | −18.821 | 2.413 | 1.00 | 10.28 | L | C |
| ATOM | 4238 | C | LEU | L | 136 | −1.080 | −17.592 | 7.052 | 1.00 | 22.56 | L | C |
| ATOM | 4239 | O | LEU | L | 136 | −0.930 | −16.374 | 6.947 | 1.00 | 26.03 | L | O |
| ATOM | 4240 | N | ASN | L | 137 | −0.885 | −18.241 | 8.196 | 1.00 | 23.82 | L | N |
| ATOM | 4241 | CA | ASN | L | 137 | −0.466 | −17.523 | 9.402 | 1.00 | 28.13 | L | C |
| ATOM | 4242 | CB | ASN | L | 137 | −1.323 | −17.954 | 10.592 | 1.00 | 23.62 | L | C |
| ATOM | 4243 | CG | ASN | L | 137 | −1.350 | −16.916 | 11.691 | 1.00 | 32.34 | L | C |
| ATOM | 4244 | OD1 | ASN | L | 137 | −1.834 | −15.801 | 11.486 | 1.00 | 29.34 | L | O |
| ATOM | 4245 | ND2 | ASN | L | 137 | −0.836 | −17.273 | 12.869 | 1.00 | 24.88 | L | N |
| ATOM | 4246 | C | ASN | L | 137 | 1.010 | −17.613 | 9.792 | 1.00 | 26.58 | L | C |
| ATOM | 4247 | O | ASN | L | 137 | 1.613 | −18.692 | 9.778 | 1.00 | 27.55 | L | O |
| ATOM | 4248 | N | ASN | L | 138 | 1.563 | −16.450 | 10.130 | 1.00 | 29.75 | L | N |
| ATOM | 4249 | CA | ASN | L | 138 | 2.947 | −16.272 | 10.585 | 1.00 | 26.46 | L | C |
| ATOM | 4250 | CB | ASN | L | 138 | 2.996 | −16.489 | 12.100 | 1.00 | 29.78 | L | C |
| ATOM | 4251 | CG | ASN | L | 138 | 2.168 | −15.458 | 12.856 | 1.00 | 31.82 | L | C |
| ATOM | 4252 | OD1 | ASN | L | 138 | 0.957 | −15.418 | 12.722 | 1.00 | 34.95 | L | O |
| ATOM | 4253 | ND2 | ASN | L | 138 | 2.832 | −14.576 | 13.602 | 1.00 | 35.46 | L | N |
| ATOM | 4254 | C | ASN | L | 138 | 4.053 | −17.062 | 9.859 | 1.00 | 28.76 | L | C |
| ATOM | 4255 | O | ASN | L | 138 | 4.661 | −17.978 | 10.427 | 1.00 | 22.52 | L | O |
| ATOM | 4256 | N | PHE | L | 139 | 4.338 | −16.658 | 8.619 | 1.00 | 21.63 | L | N |
| ATOM | 4257 | CA | PHE | L | 139 | 5.345 | −17.316 | 7.799 | 1.00 | 22.95 | L | C |
| ATOM | 4258 | CB | PHE | L | 139 | 4.689 | −18.049 | 6.593 | 1.00 | 21.04 | L | C |
| ATOM | 4259 | CG | PHE | L | 139 | 4.025 | −17.139 | 5.582 | 1.00 | 17.75 | L | C |
| ATOM | 4260 | CD1 | PHE | L | 139 | 2.737 | −16.650 | 5.795 | 1.00 | 18.31 | L | C |
| ATOM | 4261 | CD2 | PHE | L | 139 | 4.689 | −16.769 | 4.421 | 1.00 | 15.14 | L | C |
| ATOM | 4262 | CE1 | PHE | L | 139 | 2.125 | −15.799 | 4.867 | 1.00 | 15.64 | L | C |
| ATOM | 4263 | CE2 | PHE | L | 139 | 4.084 | −15.916 | 3.480 | 1.00 | 14.36 | L | C |
| ATOM | 4264 | CZ | PHE | L | 139 | 2.801 | −15.431 | 3.706 | 1.00 | 16.41 | L | C |
| ATOM | 4265 | C | PHE | L | 139 | 6.479 | −16.400 | 7.324 | 1.00 | 23.12 | L | C |
| ATOM | 4266 | O | PHE | L | 139 | 6.363 | −15.169 | 7.355 | 1.00 | 27.10 | L | O |
| ATOM | 4267 | N | TYR | L | 140 | 7.576 | −17.022 | 6.894 | 1.00 | 23.02 | L | N |
| ATOM | 4268 | CA | TYR | L | 140 | 8.759 | −16.312 | 6.396 | 1.00 | 21.22 | L | C |
| ATOM | 4269 | CB | TYR | L | 140 | 9.637 | −15.757 | 7.537 | 1.00 | 17.43 | L | C |
| ATOM | 4270 | CG | TYR | L | 140 | 10.784 | −14.901 | 7.021 | 1.00 | 16.81 | L | C |
| ATOM | 4271 | CD1 | TYR | L | 140 | 11.961 | −15.494 | 6.558 | 1.00 | 20.24 | L | C |
| ATOM | 4272 | CE1 | TYR | L | 140 | 12.938 | −14.763 | 5.917 | 1.00 | 14.48 | L | C |
| ATOM | 4273 | CD2 | TYR | L | 140 | 10.634 | −13.526 | 6.846 | 1.00 | 13.70 | L | C |
| ATOM | 4274 | CE2 | TYR | L | 140 | 11.615 | −12.780 | 6.202 | 1.00 | 16.81 | L | C |
| ATOM | 4275 | CZ | TYR | L | 140 | 12.764 | −13.417 | 5.734 | 1.00 | 20.25 | L | C |
| ATOM | 4276 | OH | TYR | L | 140 | 13.722 | −12.722 | 5.034 | 1.00 | 24.84 | L | O |
| ATOM | 4277 | C | TYR | L | 140 | 9.570 | −17.301 | 5.573 | 1.00 | 18.39 | L | C |
| ATOM | 4278 | O | TYR | L | 140 | 9.773 | −18.436 | 5.987 | 1.00 | 18.90 | L | O |
| ATOM | 4279 | N | PRO | L | 141 | 10.063 | −16.877 | 4.406 | 1.00 | 15.57 | L | N |
| ATOM | 4280 | CD | PRO | L | 141 | 10.906 | −17.767 | 3.588 | 1.00 | 19.89 | L | C |
| ATOM | 4281 | CA | PRO | L | 141 | 9.939 | −15.560 | 3.777 | 1.00 | 20.71 | L | C |
| ATOM | 4282 | CB | PRO | L | 141 | 10.986 | −15.622 | 2.662 | 1.00 | 19.16 | L | C |
| ATOM | 4283 | CG | PRO | L | 141 | 10.945 | −17.056 | 2.251 | 1.00 | 17.21 | L | C |
| ATOM | 4284 | C | PRO | L | 141 | 8.547 | −15.240 | 3.221 | 1.00 | 26.17 | L | C |
| ATOM | 4285 | O | PRO | L | 141 | 7.629 | −16.050 | 3.326 | 1.00 | 26.11 | L | O |
| ATOM | 4286 | N | ARG | L | 142 | 8.435 | −14.069 | 2.594 | 1.00 | 31.17 | L | N |
| ATOM | 4287 | CA | ARG | L | 142 | 7.192 | −13.559 | 2.017 | 1.00 | 34.61 | L | C |
| ATOM | 4288 | CB | ARG | L | 142 | 7.395 | −12.126 | 1.523 | 1.00 | 36.43 | L | C |
| ATOM | 4289 | CG | ARG | L | 142 | 6.309 | −11.154 | 1.958 | 1.00 | 42.89 | L | C |
| ATOM | 4290 | CD | ARG | L | 142 | 4.960 | −11.534 | 1.426 | 1.00 | 39.73 | L | C |
| ATOM | 4291 | NE | ARG | L | 142 | 4.562 | −10.704 | 0.297 | 1.00 | 46.47 | L | N |
| ATOM | 4292 | CZ | ARG | L | 142 | 3.581 | −11.021 | −0.541 | 1.00 | 47.07 | L | C |
| ATOM | 4293 | NH1 | ARG | L | 142 | 2.915 | −12.160 | −0.375 | 1.00 | 41.74 | L | N |
| ATOM | 4294 | NH2 | ARG | L | 142 | 3.233 | −10.180 | −1.508 | 1.00 | 44.75 | L | N |
| ATOM | 4295 | C | ARG | L | 142 | 6.596 | −14.381 | 0.882 | 1.00 | 32.07 | L | C |
| ATOM | 4296 | O | ARG | L | 142 | 5.394 | −14.616 | 0.861 | 1.00 | 38.76 | L | O |
| ATOM | 4297 | N | GLU | L | 143 | 7.420 | −14.783 | −0.078 | 1.00 | 36.92 | L | N |
| ATOM | 4298 | CA | GLU | L | 143 | 6.937 | −15.558 | −1.213 | 1.00 | 38.62 | L | C |
| ATOM | 4299 | CB | GLU | L | 143 | 8.100 | −15.997 | −2.116 | 1.00 | 43.41 | L | C |
| ATOM | 4300 | CG | GLU | L | 143 | 7.650 | −16.745 | −3.386 | 1.00 | 55.36 | L | C |
| ATOM | 4301 | CD | GLU | L | 143 | 8.791 | −17.065 | −4.349 | 1.00 | 62.16 | L | C |
| ATOM | 4302 | OE1 | GLU | L | 143 | 9.595 | −16.149 | −4.658 | 1.00 | 64.31 | L | O |
| ATOM | 4303 | OE2 | GLU | L | 143 | 8.872 | −18.233 | −4.809 | 1.00 | 65.55 | L | O |
| ATOM | 4304 | C | GLU | L | 143 | 6.095 | −16.775 | −0.813 | 1.00 | 38.62 | L | C |
| ATOM | 4305 | O | GLU | L | 143 | 6.547 | −17.643 | −0.073 | 1.00 | 33.02 | L | O |
| ATOM | 4306 | N | ALA | L | 144 | 4.866 | −16.819 | −1.315 | 1.00 | 29.96 | L | N |
| ATOM | 4307 | CA | ALA | L | 144 | 3.964 | −17.920 | −1.039 | 1.00 | 29.61 | L | C |
| ATOM | 4308 | CB | ALA | L | 144 | 3.221 | −17.675 | 0.242 | 1.00 | 22.30 | L | C |
| ATOM | 4309 | C | ALA | L | 144 | 2.989 | −18.043 | −2.199 | 1.00 | 32.76 | L | C |
| ATOM | 4310 | O | ALA | L | 144 | 2.485 | −17.033 | −2.706 | 1.00 | 33.94 | L | O |
| ATOM | 4311 | N | LYS | L | 145 | 2.772 | −19.268 | −2.663 | 1.00 | 30.87 | L | N |
| ATOM | 4312 | CA | LYS | L | 145 | 1.841 | −19.490 | −3.759 | 1.00 | 35.19 | L | C |
| ATOM | 4313 | CB | LYS | L | 145 | 2.467 | −20.372 | −4.849 | 1.00 | 42.02 | L | C |
| ATOM | 4314 | CG | LYS | L | 145 | 1.714 | −20.421 | −6.197 | 1.00 | 45.19 | L | C |
| ATOM | 4315 | CD | LYS | L | 145 | 2.430 | −21.404 | −7.176 | 1.00 | 53.92 | L | C |
| ATOM | 4316 | CE | LYS | L | 145 | 1.820 | −21.459 | −8.595 | 1.00 | 54.68 | L | C |
| ATOM | 4317 | NZ | LYS | L | 145 | 1.922 | −20.181 | −9.389 | 1.00 | 51.57 | L | N |
| ATOM | 4318 | C | LYS | L | 145 | 0.585 | −20.134 | −3.196 | 1.00 | 33.53 | L | C |
| ATOM | 4319 | O | LYS | L | 145 | 0.645 | −21.193 | −2.553 | 1.00 | 24.86 | L | O |
| ATOM | 4320 | N | VAL | L | 146 | −0.528 | −19.423 | −3.353 | 1.00 | 26.20 | L | N |
| ATOM | 4321 | CA | VAL | L | 146 | −1.819 | −19.910 | −2.896 | 1.00 | 31.96 | L | C |
| ATOM | 4322 | CB | VAL | L | 146 | −2.494 | −18.921 | −1.938 | 1.00 | 35.17 | L | C |
| ATOM | 4323 | CG1 | VAL | L | 146 | −3.783 | −19.520 | −1.391 | 1.00 | 32.93 | L | C |
| ATOM | 4324 | CG2 | VAL | L | 146 | −1.537 | −18.561 | −0.803 | 1.00 | 36.01 | L | C |
| ATOM | 4325 | C | VAL | L | 146 | −2.727 | −20.145 | −4.097 | 1.00 | 34.35 | L | C |
| ATOM | 4326 | O | VAL | L | 146 | −3.072 | −19.213 | −4.833 | 1.00 | 34.10 | L | O |
| ATOM | 4327 | N | GLN | L | 147 | −3.045 | −21.408 | −4.342 | 1.00 | 38.79 | L | N |
| ATOM | 4328 | CA | GLN | L | 147 | −3.926 | −21.746 | −5.445 | 1.00 | 42.36 | L | C |
| ATOM | 4329 | CB | GLN | L | 147 | −3.250 | −22.702 | −6.418 | 1.00 | 46.61 | L | C |
| ATOM | 4330 | CG | GLN | L | 147 | −2.216 | −22.009 | −7.288 | 1.00 | 51.56 | L | C |
| ATOM | 4331 | CD | GLN | L | 147 | −1.701 | −22.894 | −8.400 | 1.00 | 56.85 | L | C |
| ATOM | 4332 | OE1 | GLN | L | 147 | −1.714 | −22.505 | −9.575 | 1.00 | 56.05 | L | O |
| ATOM | 4333 | NE2 | GLN | L | 147 | −1.242 | −24.094 | −8.040 | 1.00 | 53.76 | L | N |
| ATOM | 4334 | C | GLN | L | 147 | −5.221 | −22.337 | −4.949 | 1.00 | 42.60 | L | C |
| ATOM | 4335 | O | GLN | L | 147 | −5.244 | −23.127 | −4.006 | 1.00 | 44.70 | L | O |
| ATOM | 4336 | N | TRP | L | 148 | −6.311 | −21.899 | −5.557 | 1.00 | 41.00 | L | N |
| ATOM | 4337 | CA | TRP | L | 148 | −7.626 | −22.394 | −5.196 | 1.00 | 37.17 | L | C |
| ATOM | 4338 | CB | TRP | L | 148 | −8.636 | −21.263 | −5.292 | 1.00 | 31.51 | L | C |
| ATOM | 4339 | CG | TRP | L | 148 | −8.641 | −20.414 | −4.090 | 1.00 | 28.42 | L | C |
| ATOM | 4340 | CD2 | TRP | L | 148 | −9.238 | −20.734 | −2.835 | 1.00 | 29.77 | L | C |
| ATOM | 4341 | CE2 | TRP | L | 148 | −9.035 | −19.624 | −1.984 | 1.00 | 29.62 | L | C |
| ATOM | 4342 | CE3 | TRP | L | 148 | −9.924 | −21.857 | −2.343 | 1.00 | 29.51 | L | C |
| ATOM | 4343 | CD1 | TRP | L | 148 | −8.106 | −19.167 | −3.959 | 1.00 | 25.61 | L | C |
| ATOM | 4344 | NE1 | TRP | L | 148 | −8.344 | −18.683 | −2.698 | 1.00 | 29.94 | L | N |
| ATOM | 4345 | CZ2 | TRP | L | 148 | −9.491 | −19.599 | −0.663 | 1.00 | 31.99 | L | C |
| ATOM | 4346 | CZ3 | TRP | L | 148 | −10.376 | −21.836 | −1.035 | 1.00 | 30.69 | L | C |
| ATOM | 4347 | CH2 | TRP | L | 148 | −10.157 | −20.707 | −0.204 | 1.00 | 33.96 | L | C |
| ATOM | 4348 | C | TRP | L | 148 | −8.034 | −23.570 | −6.078 | 1.00 | 36.71 | L | C |
| ATOM | 4349 | O | TRP | L | 148 | −7.803 | −23.557 | −7.290 | 1.00 | 36.02 | L | O |
| ATOM | 4350 | N | LYS | L | 149 | −8.617 | −24.595 | −5.467 | 1.00 | 38.04 | L | N |
| ATOM | 4351 | CA | LYS | L | 149 | −9.039 | −25.770 | −6.215 | 1.00 | 44.38 | L | C |
| ATOM | 4352 | CB | LYS | L | 149 | −8.091 | −26.946 | −5.959 | 1.00 | 48.57 | L | C |
| ATOM | 4353 | CG | LYS | L | 149 | −6.686 | −26.764 | −6.539 | 1.00 | 50.51 | L | C |
| ATOM | 4354 | CD | LYS | L | 149 | −5.761 | −27.933 | −6.180 | 1.00 | 52.78 | L | C |
| ATOM | 4355 | CE | LYS | L | 149 | −4.350 | −27.703 | −6.714 | 1.00 | 51.87 | L | C |
| ATOM | 4356 | NZ | LYS | L | 149 | −3.478 | −28.894 | −6.524 | 1.00 | 50.02 | L | N |
| ATOM | 4357 | C | LYS | L | 149 | −10.454 | −26.139 | −5.832 | 1.00 | 45.39 | L | C |
| ATOM | 4358 | O | LYS | L | 149 | −10.739 | −26.405 | −4.660 | 1.00 | 42.84 | L | O |
| ATOM | 4359 | N | VAL | L | 150 | −11.339 | −26.091 | −6.824 | 1.00 | 45.70 | L | N |
| ATOM | 4360 | CA | VAL | L | 150 | −12.759 | −26.414 | −6.647 | 1.00 | 49.72 | L | C |
| ATOM | 4361 | CB | VAL | L | 150 | −13.660 | −25.263 | −7.188 | 1.00 | 50.34 | L | C |
| ATOM | 4362 | CG1 | VAL | L | 150 | −15.130 | −25.539 | −6.885 | 1.00 | 51.57 | L | C |
| ATOM | 4363 | CG2 | VAL | L | 150 | −13.247 | −23.934 | −6.568 | 1.00 | 49.30 | L | C |
| ATOM | 4364 | C | VAL | L | 150 | −13.021 | −27.712 | −7.418 | 1.00 | 49.74 | L | C |
| ATOM | 4365 | O | VAL | L | 150 | −13.029 | −27.719 | −8.653 | 1.00 | 51.54 | L | O |
| ATOM | 4366 | N | ASP | L | 151 | −13.228 | −28.805 | −6.684 | 1.00 | 52.11 | L | N |
| ATOM | 4367 | CA | ASP | L | 151 | −13.428 | −30.125 | −7.284 | 1.00 | 52.55 | L | C |
| ATOM | 4368 | CB | ASP | L | 151 | −14.760 | −30.209 | −8.041 | 1.00 | 49.59 | L | C |
| ATOM | 4369 | CG | ASP | L | 151 | −15.945 | −30.472 | −7.120 | 1.00 | 49.81 | L | C |
| ATOM | 4370 | OD1 | ASP | L | 151 | −15.739 | −30.939 | −5.982 | 1.00 | 45.62 | L | O |
| ATOM | 4371 | OD2 | ASP | L | 151 | −17.094 | −30.228 | −7.540 | 1.00 | 53.11 | L | O |
| ATOM | 4372 | C | ASP | L | 151 | −12.247 | −30.430 | −8.203 | 1.00 | 52.92 | L | C |
| ATOM | 4373 | O | ASP | L | 151 | −12.426 | −30.785 | −9.374 | 1.00 | 56.01 | L | O |
| ATOM | 4374 | N | ASN | L | 152 | −11.041 | −30.207 | −7.677 | 1.00 | 51.23 | L | N |
| ATOM | 4375 | CA | ASN | L | 152 | −9.799 | −30.453 | −8.408 | 1.00 | 52.62 | L | C |
| ATOM | 4376 | CB | ASN | L | 152 | −9.769 | −31.918 | −8.888 | 1.00 | 61.77 | L | C |
| ATOM | 4377 | CG | ASN | L | 152 | −8.401 | −32.355 | −9.392 | 1.00 | 65.86 | L | C |
| ATOM | 4378 | OD1 | ASN | L | 152 | −7.461 | −32.520 | −8.608 | 1.00 | 71.35 | L | O |
| ATOM | 4379 | ND2 | ASN | L | 152 | −8.290 | −32.563 | −10.704 | 1.00 | 68.04 | L | N |
| ATOM | 4380 | C | ASN | L | 152 | −9.575 | −29.497 | −9.585 | 1.00 | 50.92 | L | C |
| ATOM | 4381 | O | ASN | L | 152 | −8.625 | −29.661 | −10.351 | 1.00 | 53.80 | L | O |
| ATOM | 4382 | N | ALA | L | 153 | −10.454 | −28.514 | −9.746 | 1.00 | 47.36 | L | N |
| ATOM | 4383 | CA | ALA | L | 153 | −10.308 | −27.550 | −10.832 | 1.00 | 46.66 | L | C |
| ATOM | 4384 | CB | ALA | L | 153 | −11.671 | −27.211 | −11.431 | 1.00 | 45.45 | L | C |
| ATOM | 4385 | C | ALA | L | 153 | −9.605 | −26.285 | −10.346 | 1.00 | 43.03 | L | C |
| ATOM | 4386 | O | ALA | L | 153 | −10.038 | −25.662 | −9.382 | 1.00 | 37.89 | L | O |
| ATOM | 4387 | N | LEU | L | 154 | −8.517 | −25.931 | −11.023 | 1.00 | 46.70 | L | N |
| ATOM | 4388 | CA | LEU | L | 154 | −7.714 | −24.749 | −10.704 | 1.00 | 46.58 | L | C |
| ATOM | 4389 | CB | LEU | L | 154 | −6.418 | −24.756 | −11.515 | 1.00 | 45.70 | L | C |
| ATOM | 4390 | CG | LEU | L | 154 | −5.127 | −24.561 | −10.728 | 1.00 | 48.99 | L | C |
| ATOM | 4391 | CD1 | LEU | L | 154 | −4.690 | −25.903 | −10.154 | 1.00 | 44.24 | L | C |
| ATOM | 4392 | CD2 | LEU | L | 154 | −4.046 | −24.004 | −11.646 | 1.00 | 48.60 | L | C |
| ATOM | 4393 | C | LEU | L | 154 | −8.475 | −23.461 | −11.016 | 1.00 | 49.09 | L | C |
| ATOM | 4394 | O | LEU | L | 154 | −8.943 | −23.262 | −12.148 | 1.00 | 48.07 | L | O |
| ATOM | 4395 | N | GLN | L | 155 | −8.553 | −22.577 | −10.022 | 1.00 | 41.81 | L | N |
| ATOM | 4396 | CA | GLN | L | 155 | −9.257 | −21.310 | −10.166 | 1.00 | 40.59 | L | C |
| ATOM | 4397 | CB | GLN | L | 155 | −9.848 | −20.891 | −8.826 | 1.00 | 39.38 | L | C |
| ATOM | 4398 | CG | GLN | L | 155 | −10.889 | −21.853 | −8.308 | 1.00 | 40.51 | L | C |
| ATOM | 4399 | CD | GLN | L | 155 | −11.973 | −22.119 | −9.335 | 1.00 | 40.79 | L | C |
| ATOM | 4400 | OE1 | GLN | L | 155 | −12.958 | −21.377 | −9.426 | 1.00 | 36.60 | L | O |
| ATOM | 4401 | NE2 | GLN | L | 155 | −11.785 | −23.173 | −10.133 | 1.00 | 37.70 | L | N |
| ATOM | 4402 | C | GLN | L | 155 | −8.369 | −20.198 | −10.685 | 1.00 | 41.49 | L | C |
| ATOM | 4403 | O | GLN | L | 155 | −7.154 | −20.235 | −10.506 | 1.00 | 42.35 | L | O |
| ATOM | 4404 | N | SER | L | 156 | −8.993 | −19.196 | −11.302 | 1.00 | 39.38 | L | N |
| ATOM | 4405 | CA | SER | L | 156 | −8.283 | −18.046 | −11.847 | 1.00 | 40.37 | L | C |
| ATOM | 4406 | CB | SER | L | 156 | −7.692 | −18.398 | −13.217 | 1.00 | 43.50 | L | C |
| ATOM | 4407 | OG | SER | L | 156 | −7.202 | −17.239 | −13.886 | 1.00 | 45.86 | L | O |
| ATOM | 4408 | C | SER | L | 156 | −9.206 | −16.828 | −11.978 | 1.00 | 42.72 | L | C |
| ATOM | 4409 | O | SER | L | 156 | −10.343 | −16.953 | −12.435 | 1.00 | 49.85 | L | O |
| ATOM | 4410 | N | GLY | L | 157 | −8.720 | −15.659 | −11.562 | 1.00 | 40.34 | L | N |
| ATOM | 4411 | CA | GLY | L | 157 | −9.505 | −14.440 | −11.668 | 1.00 | 32.09 | L | C |
| ATOM | 4412 | C | GLY | L | 157 | −10.617 | −14.193 | −10.656 | 1.00 | 34.70 | L | C |
| ATOM | 4413 | O | GLY | L | 157 | −11.249 | −13.134 | −10.681 | 1.00 | 37.94 | L | O |
| ATOM | 4414 | N | ASN | L | 158 | −10.872 | −15.138 | −9.761 | 1.00 | 29.92 | L | N |
| ATOM | 4415 | CA | ASN | L | 158 | −11.935 | −14.948 | −8.777 | 1.00 | 32.23 | L | C |
| ATOM | 4416 | CB | ASN | L | 158 | −13.073 | −15.969 | −8.990 | 1.00 | 35.97 | L | C |
| ATOM | 4417 | CG | ASN | L | 158 | −12.564 | −17.390 | −9.262 | 1.00 | 41.52 | L | C |
| ATOM | 4418 | OD1 | ASN | L | 158 | −11.359 | −17.629 | −9.381 | 1.00 | 43.82 | L | O |
| ATOM | 4419 | ND2 | ASN | L | 158 | −13.491 | −18.332 | −9.380 | 1.00 | 41.04 | L | N |
| ATOM | 4420 | C | ASN | L | 158 | −11.463 | −14.921 | −7.319 | 1.00 | 29.39 | L | C |
| ATOM | 4421 | O | ASN | L | 158 | −12.249 | −15.141 | −6.390 | 1.00 | 30.02 | L | O |
| ATOM | 4422 | N | SER | L | 159 | −10.174 | −14.652 | −7.125 | 1.00 | 29.57 | L | N |
| ATOM | 4423 | CA | SER | L | 159 | −9.601 | −14.559 | −5.788 | 1.00 | 29.01 | L | C |
| ATOM | 4424 | CB | SER | L | 159 | −8.752 | −15.793 | −5.474 | 1.00 | 30.42 | L | C |
| ATOM | 4425 | OG | SER | L | 159 | −7.667 | −15.925 | −6.380 | 1.00 | 36.09 | L | O |
| ATOM | 4426 | C | SER | L | 1529 | −8.759 | −13.293 | −5.628 | 1.00 | 27.61 | L | C |
| ATOM | 4427 | O | SER | L | 159 | −8.308 | −12.700 | −6.608 | 1.00 | 33.69 | L | O |
| ATOM | 4428 | N | GLN | L | 160 | −8.604 | −12.855 | −4.387 | 1.00 | 25.34 | L | N |
| ATOM | 4429 | CA | GLN | L | 160 | −7.789 | −11.691 | −4.070 | 1.00 | 28.24 | L | C |
| ATOM | 4430 | CB | GLN | L | 160 | −8.651 | −10.441 | −3.941 | 1.00 | 20.62 | L | C |
| ATOM | 4431 | CG | GLN | L | 160 | −9.090 | −9.877 | −5.282 | 1.00 | 30.31 | L | C |
| ATOM | 4432 | CD | GLN | L | 160 | −9.817 | −8.554 | −5.146 | 1.00 | 37.08 | L | C |
| ATOM | 4433 | OE1 | GLN | L | 160 | −10.570 | −8.347 | −4.198 | 1.00 | 42.20 | L | O |
| ATOM | 4434 | NE2 | GLN | L | 160 | −9.576 | −7.640 | −6.082 | 1.00 | 41.74 | L | N |
| ATOM | 4435 | C | GLN | L | 160 | −7.006 | −11.943 | −2.785 | 1.00 | 25.20 | L | C |
| ATOM | 4436 | O | GLN | L | 160 | −7.480 | −12.635 | −1.881 | 1.00 | 25.70 | L | O |
| ATOM | 4437 | N | GLU | L | 161 | −5.763 | −11.493 | −2.742 | 1.00 | 25.48 | L | N |
| ATOM | 4438 | CA | GLU | L | 161 | −5.004 | −11.684 | −1.521 | 1.00 | 23.98 | L | C |
| ATOM | 4439 | CB | GLU | L | 161 | −3.844 | −12.654 | −1.680 | 1.00 | 26.40 | L | C |
| ATOM | 4440 | CG | GLU | L | 161 | −3.045 | −12.535 | −2.914 | 1.00 | 30.50 | L | C |
| ATOM | 4441 | CD | GLU | L | 161 | −1.921 | −13.565 | −2.928 | 1.00 | 43.03 | L | C |
| ATOM | 4442 | OE1 | GLU | L | 161 | −2.185 | −14.756 | −2.623 | 1.00 | 40.29 | L | O |
| ATOM | 4443 | OE2 | GLU | L | 161 | −0.767 | −13.178 | −3.223 | 1.00 | 45.07 | L | O |
| ATOM | 4444 | C | GLU | L | 161 | −4.579 | −10.413 | −0.859 | 1.00 | 19.13 | L | C |
| ATOM | 4445 | O | GLU | L | 161 | −4.593 | −9.345 | −1.450 | 1.00 | 23.28 | L | O |
| ATOM | 4446 | N | SER | L | 162 | −4.246 | −10.534 | 0.409 | 1.00 | 26.44 | L | N |
| ATOM | 4447 | CA | SER | L | 162 | −3.834 | −9.395 | 1.211 | 1.00 | 24.31 | L | C |
| ATOM | 4448 | CB | SER | L | 162 | −5.056 | −8.851 | 1.965 | 1.00 | 21.25 | L | C |
| ATOM | 4449 | OG | SER | L | 162 | −4.770 | −7.672 | 2.696 | 1.00 | 21.46 | L | O |
| ATOM | 4450 | C | SER | L | 162 | −2.776 | −9.902 | 2.183 | 1.00 | 21.26 | L | C |
| ATOM | 4451 | O | SER | L | 162 | −2.899 | −10.997 | 2.734 | 1.00 | 18.72 | L | O |
| ATOM | 4452 | N | VAL | L | 163 | −1.722 | −9.116 | 2.368 | 1.00 | 23.64 | L | N |
| ATOM | 4453 | CA | VAL | L | 163 | −0.644 | −9.512 | 3.259 | 1.00 | 24.07 | L | C |
| ATOM | 4454 | CB | VAL | L | 163 | 0.669 | −9.700 | 2.502 | 1.00 | 24.98 | L | C |
| ATOM | 4455 | CG1 | VAL | L | 163 | 1.651 | −10.470 | 3.360 | 1.00 | 26.07 | L | C |
| ATOM | 4456 | CG2 | VAL | L | 163 | 0.430 | −10.418 | 1.205 | 1.00 | 33.89 | L | C |
| ATOM | 4457 | C | VAL | L | 163 | −0.399 | −8.433 | 4.274 | 1.00 | 24.56 | L | C |
| ATOM | 4458 | O | VAL | L | 163 | −0.529 | −7.248 | 3.973 | 1.00 | 23.88 | L | O |
| ATOM | 4459 | N | THR | L | 164 | −0.062 | −8.842 | 5.488 | 1.00 | 18.82 | L | N |
| ATOM | 4460 | CA | THR | L | 164 | 0.246 | −7.874 | 6.530 | 1.00 | 29.84 | L | C |
| ATOM | 4461 | CB | THR | L | 164 | 0.107 | −8.485 | 7.953 | 1.00 | 26.74 | L | C |
| ATOM | 4462 | OG1 | THR | L | 164 | 1.037 | −9.563 | 8.105 | 1.00 | 33.17 | L | O |
| ATOM | 4463 | CG2 | THR | L | 164 | −1.295 | −9.007 | 8.180 | 1.00 | 23.53 | L | C |
| ATOM | 4464 | C | THR | L | 164 | 1.713 | −7.463 | 6.342 | 1.00 | 31.73 | L | C |
| ATOM | 4465 | O | THR | L | 164 | 2.424 | −8.013 | 5.494 | 1.00 | 32.77 | L | O |
| ATOM | 4466 | N | GLU | L | 165 | 2.127 | −6.437 | 7.074 | 1.00 | 31.31 | L | N |
| ATOM | 4467 | CA | GLU | L | 165 | 3.508 | −5.980 | 7.055 | 1.00 | 26.27 | L | C |
| ATOM | 4468 | CB | GLU | L | 165 | 3.582 | −4.508 | 7.514 | 1.00 | 25.47 | L | C |
| ATOM | 4469 | CG | GLU | L | 165 | 3.062 | −3.481 | 6.477 | 1.00 | 37.05 | L | C |
| ATOM | 4470 | CD | GLU | L | 165 | 2.821 | −2.062 | 7.059 | 1.00 | 47.67 | L | C |
| ATOM | 4471 | OE1 | GLU | L | 165 | 3.117 | −1.831 | 8.257 | 1.00 | 54.59 | L | O |
| ATOM | 4472 | OE2 | GLU | L | 165 | 2.329 | −1.164 | 6.321 | 1.00 | 44.45 | L | O |
| ATOM | 4473 | C | GLU | L | 165 | 4.197 | −6.923 | 8.058 | 1.00 | 22.51 | L | C |
| ATOM | 4474 | O | GLU | L | 165 | 3.530 | −7.583 | 8.856 | 1.00 | 21.84 | L | O |
| ATOM | 4475 | N | GLN | L | 166 | 5.515 | −7.031 | 7.987 | 1.00 | 26.32 | L | N |
| ATOM | 4476 | CA | GLN | L | 166 | 6.265 | −7.890 | 8.899 | 1.00 | 20.38 | L | C |
| ATOM | 4477 | CB | GLN | L | 166 | 7.753 | −7.641 | 8.706 | 1.00 | 21.85 | L | C |
| ATOM | 4478 | CG | GLN | L | 166 | 8.435 | −8.716 | 7.938 | 1.00 | 23.04 | L | C |
| ATOM | 4479 | CD | GLN | L | 166 | 9.931 | −8.533 | 7.869 | 1.00 | 24.62 | L | C |
| ATOM | 4480 | OE1 | GLN | L | 166 | 10.421 | −7.433 | 7.615 | 1.00 | 27.52 | L | O |
| ATOM | 4481 | NE2 | GLN | L | 166 | 10.666 | −9.619 | 8.082 | 1.00 | 22.29 | L | N |
| ATOM | 4482 | C | GLN | L | 166 | 5.903 | −7.621 | 10.361 | 1.00 | 24.03 | L | C |
| ATOM | 4483 | O | GLN | L | 166 | 5.891 | −6.471 | 10.794 | 1.00 | 21.65 | L | O |
| ATOM | 4484 | N | ASP | L | 167 | 5.618 | −8.672 | 11.121 | 1.00 | 24.56 | L | N |
| ATOM | 4485 | CA | ASP | L | 167 | 5.250 | −8.498 | 12.519 | 1.00 | 29.86 | L | C |
| ATOM | 4486 | CB | ASP | L | 167 | 4.859 | −9.838 | 13.141 | 1.00 | 33.66 | L | C |
| ATOM | 4487 | CG | ASP | L | 167 | 4.599 | −9.736 | 14.645 | 1.00 | 42.87 | L | C |
| ATOM | 4488 | OD1 | ASP | L | 167 | 3.894 | −8.791 | 15.072 | 1.00 | 49.85 | L | O |
| ATOM | 4489 | OD2 | ASP | L | 167 | 5.108 | −10.596 | 15.404 | 1.00 | 40.03 | L | O |
| ATOM | 4490 | C | ASP | L | 167 | 6.412 | −7.878 | 13.290 | 1.00 | 34.92 | L | C |
| ATOM | 4491 | O | ASP | L | 167 | 7.565 | −8.230 | 13.055 | 1.00 | 37.94 | L | O |
| ATOM | 4492 | N | SER | L | 168 | 6.103 | −6.959 | 14.204 | 1.00 | 37.68 | L | N |
| ATOM | 4493 | CA | SER | L | 168 | 7.136 | −6.290 | 14.996 | 1.00 | 39.71 | L | C |
| ATOM | 4494 | CB | SER | L | 168 | 6.570 | −5.052 | 15.704 | 1.00 | 36.06 | L | C |
| ATOM | 4495 | OG | SER | L | 168 | 5.575 | −5.407 | 16.646 | 1.00 | 37.99 | L | O |
| ATOM | 4496 | C | SER | L | 168 | 7.841 | −7.221 | 16.003 | 1.00 | 42.25 | L | C |
| ATOM | 4497 | O | SER | L | 168 | 9.002 | −7.001 | 16.356 | 1.00 | 37.25 | L | O |
| ATOM | 4498 | N | LYS | L | 169 | 7.161 | −8.284 | 16.423 | 1.00 | 43.35 | L | N |
| ATOM | 4499 | CA | LYS | L | 169 | 7.748 | −9.224 | 17.368 | 1.00 | 45.17 | L | C |
| ATOM | 4500 | CB | LYS | L | 169 | 6.677 | −9.738 | 18.338 | 1.00 | 49.54 | L | C |
| ATOM | 4501 | CG | LYS | L | 169 | 6.194 | −8.668 | 19.328 | 1.00 | 57.46 | L | C |
| ATOM | 4502 | CD | LYS | L | 169 | 4.990 | −9.118 | 20.174 | 1.00 | 60.61 | L | C |
| ATOM | 4503 | CE | LYS | L | 169 | 3.660 | −8.991 | 19.405 | 1.00 | 65.80 | L | C |
| ATOM | 4504 | NZ | LYS | L | 169 | 2.453 | −9.363 | 20.217 | 1.00 | 60.82 | L | N |
| ATOM | 4505 | C | LYS | L | 169 | 8.521 | −10.383 | 16.725 | 1.00 | 44.38 | L | C |
| ATOM | 4506 | O | LYS | L | 169 | 9.715 | −10.541 | 16.988 | 1.00 | 46.59 | L | O |
| ATOM | 4507 | N | ASP | L | 170 | 7.869 | −11.170 | 15.866 | 1.00 | 39.76 | L | N |
| ATOM | 4508 | CA | ASP | L | 170 | 8.540 | −12.309 | 15.234 | 1.00 | 31.66 | L | C |
| ATOM | 4509 | CB | ASP | L | 170 | 7.709 | −13.586 | 15.404 | 1.00 | 36.85 | L | C |
| ATOM | 4510 | CG | ASP | L | 170 | 6.421 | −13.579 | 14.585 | 1.00 | 41.74 | L | C |
| ATOM | 4511 | OD1 | ASP | L | 170 | 6.237 | −12.674 | 13.741 | 1.00 | 39.67 | L | O |
| ATOM | 4512 | OD2 | ASP | L | 170 | 5.598 | −14.510 | 14.774 | 1.00 | 44.04 | L | O |
| ATOM | 4513 | C | ASP | L | 170 | 9.009 | −12.155 | 13.779 | 1.00 | 30.83 | L | C |
| ATOM | 4514 | O | ASP | L | 170 | 9.544 | −13.107 | 13.185 | 1.00 | 22.80 | L | O |
| ATOM | 4515 | N | SER | L | 171 | 8.777 | −10.980 | 13.196 | 1.00 | 27.28 | L | N |
| ATOM | 4516 | CA | SER | L | 171 | 9.205 | −10.687 | 11.826 | 1.00 | 28.16 | L | C |
| ATOM | 4517 | CB | SER | L | 171 | 10.735 | −10.650 | 11.744 | 1.00 | 26.91 | L | C |
| ATOM | 4518 | OG | SER | L | 171 | 11.292 | −9.698 | 12.644 | 1.00 | 33.05 | L | O |
| ATOM | 4519 | C | SER | L | 171 | 8.640 | −11.615 | 10.743 | 1.00 | 30.30 | L | C |
| ATOM | 4520 | O | SER | L | 171 | 9.223 | −11.761 | 9.665 | 1.00 | 31.41 | L | O |
| ATOM | 4521 | N | THR | L | 172 | 7.490 | −12.221 | 11.016 | 1.00 | 30.53 | L | N |
| ATOM | 4522 | CA | THR | L | 172 | 6.859 | −13.095 | 10.035 | 1.00 | 24.29 | L | C |
| ATOM | 4523 | CB | THR | L | 172 | 6.165 | −14.295 | 10.704 | 1.00 | 24.35 | L | C |
| ATOM | 4524 | OG1 | THR | L | 172 | 5.134 | −13.824 | 11.566 | 1.00 | 21.78 | L | O |
| ATOM | 4525 | CG2 | THR | L | 172 | 7.150 | −15.146 | 11.503 | 1.00 | 18.50 | L | C |
| ATOM | 4526 | C | THR | L | 172 | 5.810 | −12.313 | 9.242 | 1.00 | 24.07 | L | C |
| ATOM | 4527 | O | THR | L | 172 | 5.626 | −11.108 | 9.439 | 1.00 | 28.24 | L | O |
| ATOM | 4528 | N | TYR | L | 173 | 5.136 | −13.009 | 8.339 | 1.00 | 22.66 | L | N |
| ATOM | 4529 | CA | TYR | L | 173 | 4.083 | −12.418 | 7.525 | 1.00 | 24.35 | L | C |
| ATOM | 4530 | CB | TYR | L | 173 | 4.508 | −12.404 | 6.060 | 1.00 | 19.74 | L | C |
| ATOM | 4531 | CG | TYR | L | 173 | 5.660 | −11.495 | 5.753 | 1.00 | 23.44 | L | C |
| ATOM | 4532 | CD1 | TYR | L | 173 | 5.437 | −10.146 | 5.457 | 1.00 | 14.39 | L | C |
| ATOM | 4533 | CE1 | TYR | L | 173 | 6.474 | −9.312 | 5.137 | 1.00 | 16.56 | L | C |
| ATOM | 4534 | CD2 | TYR | L | 173 | 6.982 | −11.986 | 5.722 | 1.00 | 17.50 | L | C |
| ATOM | 4535 | CE2 | TYR | L | 173 | 8.032 | −11.160 | 5.403 | 1.00 | 13.36 | L | C |
| ATOM | 4536 | CZ | TYR | L | 173 | 7.780 | −9.819 | 5.109 | 1.00 | 21.97 | L | C |
| ATOM | 4537 | OH | TYR | L | 173 | 8.822 | −8.971 | 4.792 | 1.00 | 19.90 | L | O |
| ATOM | 4538 | C | TYR | L | 173 | 2.803 | −13.250 | 7.625 | 1.00 | 24.88 | L | C |
| ATOM | 4539 | O | TYR | L | 173 | 2.840 | −14.441 | 7.923 | 1.00 | 24.05 | L | O |
| ATOM | 4540 | N | SER | L | 174 | 1.674 | −12.626 | 7.325 | 1.00 | 23.73 | L | N |
| ATOM | 4541 | CA | SER | L | 174 | 0.391 | −13.328 | 7.331 | 1.00 | 28.96 | L | C |
| ATOM | 4542 | CB | SER | L | 174 | −0.460 | −12.956 | 8.550 | 1.00 | 25.79 | L | C |
| ATOM | 4543 | OG | SER | L | 174 | −0.012 | −13.647 | 9.697 | 1.00 | 28.85 | L | O |
| ATOM | 4544 | C | SER | L | 174 | −0.316 | −12.943 | 6.040 | 1.00 | 27.74 | L | C |
| ATOM | 4545 | O | SER | L | 174 | −0.173 | −11.810 | 5.555 | 1.00 | 28.45 | L | O |
| ATOM | 4546 | N | LEU | L | 175 | −1.024 | −13.901 | 5.453 | 1.00 | 23.53 | L | N |
| ATOM | 4547 | CA | LEU | L | 175 | −1.721 | −13.668 | 4.196 | 1.00 | 23.01 | L | C |
| ATOM | 4548 | CB | LEU | L | 175 | −0.878 | −14.200 | 3.041 | 1.00 | 15.67 | L | C |
| ATOM | 4549 | CG | LEU | L | 175 | −1.382 | −14.156 | 1.601 | 1.00 | 18.88 | L | C |
| ATOM | 4550 | CD1 | LEU | L | 175 | −0.200 | −14.129 | 0.649 | 1.00 | 18.18 | L | C |
| ATOM | 4551 | CD2 | LEU | L | 175 | −2.243 | −15.365 | 1.308 | 1.00 | 22.56 | L | C |
| ATOM | 4552 | C | LEU | L | 175 | −3.106 | −14.309 | 4.198 | 1.00 | 27.03 | L | C |
| ATOM | 4553 | O | LEU | L | 175 | −3.346 | −15.338 | 4.841 | 1.00 | 24.53 | L | O |
| ATOM | 4554 | N | SER | L | 176 | −4.016 | −13.678 | 3.476 | 1.00 | 27.63 | L | N |
| ATOM | 4555 | CA | SER | L | 176 | −5.375 | −14.155 | 3.367 | 1.00 | 26.84 | L | C |
| ATOM | 4556 | CB | SER | L | 176 | −6.322 | −13.212 | 4.091 | 1.00 | 31.89 | L | C |
| ATOM | 4557 | OG | SER | L | 176 | −6.431 | −11.991 | 3.374 | 1.00 | 40.43 | L | O |
| ATOM | 4558 | C | SER | L | 176 | −5.728 | −14.121 | 1.908 | 1.00 | 30.43 | L | C |
| ATOM | 4559 | O | SER | L | 176 | −5.408 | −13.152 | 1.218 | 1.00 | 34.09 | L | O |
| ATOM | 4560 | N | SER | L | 177 | −6.321 | −15.207 | 1.428 | 1.00 | 31.69 | L | N |
| ATOM | 4561 | CA | SER | L | 177 | −6.772 | −15.288 | 0.049 | 1.00 | 25.56 | L | C |
| ATOM | 4562 | CB | SER | L | 177 | −6.122 | −16.450 | −0.685 | 1.00 | 29.02 | L | C |
| ATOM | 4563 | OG | SER | L | 177 | −6.586 | −16.507 | −2.018 | 1.00 | 29.83 | L | O |
| ATOM | 4564 | C | SER | L | 177 | −8.268 | −15.518 | 0.156 | 1.00 | 29.15 | L | C |
| ATOM | 4565 | O | SER | L | 177 | −8.717 | −16.372 | 0.934 | 1.00 | 26.57 | L | O |
| ATOM | 4566 | N | THR | L | 178 | −9.036 | −14.687 | −0.545 | 1.00 | 30.34 | L | N |
| ATOM | 4567 | CA | THR | L | 178 | −10.495 | −14.788 | −0.543 | 1.00 | 29.64 | L | C |
| ATOM | 4568 | CB | THR | L | 178 | −11.184 | −13.459 | −0.115 | 1.00 | 34.71 | L | C |
| ATOM | 4569 | OG1 | THR | L | 178 | −10.892 | −13.177 | 1.264 | 1.00 | 40.28 | L | O |
| ATOM | 4570 | CG2 | THR | L | 178 | −12.709 | −13.560 | −0.299 | 1.00 | 34.41 | L | C |
| ATOM | 4571 | C | THR | L | 178 | −11.029 | −15.151 | −1.919 | 1.00 | 22.14 | L | C |
| ATOM | 4572 | O | THR | L | 178 | −10.817 | −14.428 | −2.882 | 1.00 | 28.87 | L | O |
| ATOM | 4573 | N | LEU | L | 179 | −11.639 | −16.323 | −2.012 | 1.00 | 20.95 | L | N |
| ATOM | 4574 | CA | LEU | L | 179 | −12.264 | −16.796 | −3.247 | 1.00 | 23.58 | L | C |
| ATOM | 4575 | CB | LEU | L | 179 | −12.250 | −18.329 | −3.300 | 1.00 | 21.75 | L | C |
| ATOM | 4576 | CG | LEU | L | 179 | −12.928 | −18.963 | −4.517 | 1.00 | 26.12 | L | C |
| ATOM | 4577 | CD1 | LEU | L | 179 | −12.004 | −18.841 | −5.724 | 1.00 | 25.86 | L | C |
| ATOM | 4578 | CD2 | LEU | L | 179 | −13.236 | −20.427 | −4.236 | 1.00 | 25.73 | L | C |
| ATOM | 4579 | C | LEU | L | 179 | −13.707 | −16.308 | −3.153 | 1.00 | 19.72 | L | C |
| ATOM | 4580 | O | LEU | L | 179 | −14.393 | −16.621 | −2.195 | 1.00 | 21.31 | L | O |
| ATOM | 4581 | N | THR | L | 180 | −14.145 | −15.492 | −4.099 | 1.00 | 24.70 | L | N |
| ATOM | 4582 | CA | THR | L | 180 | −15.508 | −14.974 | −4.066 | 1.00 | 28.68 | L | C |
| ATOM | 4583 | CB | THR | L | 180 | −15.517 | −13.445 | −4.114 | 1.00 | 36.09 | L | C |
| ATOM | 4584 | OG1 | THR | L | 180 | −14.713 | −12.932 | −3.046 | 1.00 | 38.68 | L | O |
| ATOM | 4585 | CG2 | THR | L | 180 | −16.953 | −12.912 | −3.988 | 1.00 | 34.73 | L | C |
| ATOM | 4586 | C | THR | L | 180 | −16.375 | −15.486 | −5.214 | 1.00 | 29.95 | L | C |
| ATOM | 4587 | O | THR | L | 180 | −16.051 | −15.280 | −6.382 | 1.00 | 31.79 | L | O |
| ATOM | 4588 | N | LEU | L | 181 | −17.492 | −16.121 | −4.861 | 1.00 | 31.75 | L | N |
| ATOM | 4589 | CA | LEU | L | 181 | −18.442 | −16.673 | −5.824 | 1.00 | 31.68 | L | C |
| ATOM | 4590 | CB | LEU | L | 181 | −18.362 | −18.198 | −5.812 | 1.00 | 29.86 | L | C |
| ATOM | 4591 | CG | LEU | L | 181 | −17.056 | −18.892 | −6.178 | 1.00 | 35.74 | L | C |
| ATOM | 4592 | CD1 | LEU | L | 181 | −17.134 | −20.348 | −5.759 | 1.00 | 29.28 | L | C |
| ATOM | 4593 | CD2 | LEU | L | 181 | −16.778 | −18.744 | −7.679 | 1.00 | 30.26 | L | C |
| ATOM | 4594 | C | LEU | L | 181 | −19.884 | −16.309 | −5.480 | 1.00 | 32.05 | L | C |
| ATOM | 4595 | O | LEU | L | 181 | −20.202 | −15.985 | −4.336 | 1.00 | 31.06 | L | O |
| ATOM | 4596 | N | SER | L | 182 | −20.764 | −16.404 | −6.476 | 1.00 | 34.71 | L | N |
| ATOM | 4597 | CA | SER | L | 182 | −22.200 | −16.175 | −6.271 | 1.00 | 26.89 | L | C |
| ATOM | 4598 | CB | SER | L | 182 | −22.925 | −16.012 | −7.619 | 1.00 | 27.44 | L | C |
| ATOM | 4599 | OG | SER | L | 182 | −22.781 | −17.167 | −8.460 | 1.00 | 20.83 | L | O |
| ATOM | 4600 | C | SER | L | 182 | −22.674 | −17.466 | −5.612 | 1.00 | 29.67 | L | C |
| ATOM | 4601 | O | SER | L | 182 | −22.018 | −18.500 | −5.754 | 1.00 | 34.78 | L | O |
| ATOM | 4602 | N | LYS | L | 183 | −23.797 | −17.429 | −4.900 | 1.00 | 36.05 | L | N |
| ATOM | 4603 | CA | LYS | L | 183 | −24.315 | −18.644 | −4.265 | 1.00 | 33.15 | L | C |
| ATOM | 4604 | CB | LYS | L | 183 | −25.600 | −18.341 | −3.495 | 1.00 | 35.72 | L | C |
| ATOM | 4605 | CG | LYS | L | 183 | −26.175 | −19.537 | −2.737 | 1.00 | 38.60 | L | C |
| ATOM | 4606 | CD | LYS | L | 183 | −27.531 | −19.193 | −2.110 | 1.00 | 44.96 | L | C |
| ATOM | 4607 | CE | LYS | L | 183 | −28.300 | −20.434 | −1.671 | 1.00 | 46.55 | L | C |
| ATOM | 4608 | NZ | LYS | L | 183 | −27.637 | −21.170 | −0.570 | 1.00 | 53.82 | L | N |
| ATOM | 4609 | C | LYS | L | 183 | −24.571 | −19.707 | −5.337 | 1.00 | 33.91 | L | C |
| ATOM | 4610 | O | LYS | L | 183 | −24.250 | −20.884 | −5.157 | 1.00 | 30.10 | L | O |
| ATOM | 4611 | N | ALA | L | 184 | −25.110 | −19.271 | −6.470 | 1.00 | 31.68 | L | N |
| ATOM | 4612 | CA | ALA | L | 184 | −25.384 | −20.173 | −7.574 | 1.00 | 36.06 | L | C |
| ATOM | 4613 | CB | ALA | L | 184 | −25.905 | −19.388 | −8.785 | 1.00 | 35.77 | L | C |
| ATOM | 4614 | C | ALA | L | 184 | −24.135 | −20.961 | −7.958 | 1.00 | 33.66 | L | C |
| ATOM | 4615 | O | ALA | L | 184 | −24.154 | −22.193 | −7.959 | 1.00 | 34.79 | L | O |
| ATOM | 4616 | N | ASP | L | 185 | −23.055 | −20.252 | −8.284 | 1.00 | 40.53 | L | N |
| ATOM | 4617 | CA | ASP | L | 185 | −21.793 | −20.904 | −8.674 | 1.00 | 40.86 | L | C |
| ATOM | 4618 | CB | ASP | L | 185 | −20.761 | −19.885 | −9.154 | 1.00 | 43.82 | L | C |
| ATOM | 4619 | CG | ASP | L | 185 | −21.079 | −19.344 | −10.520 | 1.00 | 46.38 | L | C |
| ATOM | 4620 | OD1 | ASP | L | 185 | −21.232 | −20.158 | −11.448 | 1.00 | 52.43 | L | O |
| ATOM | 4621 | OD2 | ASP | L | 185 | −21.189 | −18.108 | −10.664 | 1.00 | 54.44 | L | O |
| ATOM | 4622 | C | ASP | L | 185 | −21.212 | −21.748 | −7.544 | 1.00 | 35.46 | L | C |
| ATOM | 4623 | O | ASP | L | 185 | −20.733 | −22.859 | −7.767 | 1.00 | 30.60 | L | O |
| ATOM | 4624 | N | TYR | L | 186 | −21.306 | −21.242 | −6.324 | 1.00 | 27.65 | L | N |
| ATOM | 4625 | CA | TYR | L | 186 | −20.806 | −21.980 | −5.187 | 1.00 | 34.35 | L | C |
| ATOM | 4626 | CB | TYR | L | 186 | −21.018 | −21.180 | −3.915 | 1.00 | 29.24 | L | C |
| ATOM | 4627 | CG | TYR | L | 186 | −20.559 | −21.890 | −2.669 | 1.00 | 28.84 | L | C |
| ATOM | 4628 | CD1 | TYR | L | 186 | −19.257 | −22.350 | −2.546 | 1.00 | 25.14 | L | C |
| ATOM | 4629 | CE1 | TYR | L | 186 | −18.831 | −23.006 | −1.387 | 1.00 | 27.73 | L | C |
| ATOM | 4630 | CD2 | TYR | L | 186 | −21.437 | −22.100 | −1.603 | 1.00 | 28.51 | L | C |
| ATOM | 4631 | CE2 | TYR | L | 186 | −21.025 | −22.747 | −0.445 | 1.00 | 25.55 | L | C |
| ATOM | 4632 | CZ | TYR | L | 186 | −19.720 | −23.196 | −0.342 | 1.00 | 27.11 | L | C |
| ATOM | 4633 | OH | TYR | L | 186 | −19.315 | −23.819 | 0.816 | 1.00 | 31.67 | L | O |
| ATOM | 4634 | C | TYR | L | 186 | −21.492 | −23.344 | −5.069 | 1.00 | 38.45 | L | C |
| ATOM | 4635 | O | TYR | L | 186 | −20.839 | −24.354 | −4.802 | 1.00 | 40.18 | L | O |
| ATOM | 4636 | N | GLU | L | 187 | −22.800 | −23.387 | −5.312 | 1.00 | 43.90 | L | N |
| ATOM | 4637 | CA | GLU | L | 187 | −23.545 | −24.642 | −5.194 | 1.00 | 41.35 | L | C |
| ATOM | 4638 | CB | GLU | L | 187 | −25.023 | −24.358 | −4.958 | 1.00 | 40.15 | L | C |
| ATOM | 4639 | CG | GLU | L | 187 | −25.271 | −23.734 | −3.595 | 1.00 | 43.91 | L | C |
| ATOM | 4640 | CD | GLU | L | 187 | −26.734 | −23.477 | −3.296 | 1.00 | 47.49 | L | C |
| ATOM | 4641 | OE1 | GLU | L | 187 | −27.473 | −23.057 | −4.216 | 1.00 | 43.07 | L | O |
| ATOM | 4642 | OE2 | GLU | L | 187 | −27.135 | −23.682 | −2.124 | 1.00 | 49.68 | L | O |
| ATOM | 4643 | C | GLU | L | 187 | −23.349 | −25.675 | −6.304 | 1.00 | 41.82 | L | C |
| ATOM | 4644 | O | GLU | L | 187 | −23.801 | −26.817 | −6.186 | 1.00 | 45.52 | L | O |
| ATOM | 4645 | N | LYS | L | 188 | −22.618 | −25.295 | −7.347 | 1.00 | 39.86 | L | N |
| ATOM | 4646 | CA | LYS | L | 188 | −22.345 | −26.186 | −8.472 | 1.00 | 41.80 | L | C |
| ATOM | 4647 | CB | LYS | L | 188 | −22.000 | −25.368 | −9.722 | 1.00 | 39.10 | L | C |
| ATOM | 4648 | CG | LYS | L | 188 | −23.142 | −24.556 | −10.296 | 1.00 | 38.31 | L | C |
| ATOM | 4649 | CD | LYS | L | 188 | −22.687 | −23.839 | −11.563 | 1.00 | 42.95 | L | C |
| ATOM | 4650 | CE | LYS | L | 188 | −23.807 | −23.032 | −12.199 | 1.00 | 31.44 | L | C |
| ATOM | 4651 | NZ | LYS | L | 188 | −24.978 | −23.920 | −12.487 | 1.00 | 52.10 | L | N |
| ATOM | 4652 | C | LYS | L | 188 | −21.207 | −27.186 | −8.241 | 1.00 | 44.02 | L | C |
| ATOM | 4653 | O | LYS | L | 188 | −20.935 | −28.019 | −9.112 | 1.00 | 48.16 | L | O |
| ATOM | 4654 | N | HIS | L | 189 | −20.514 | −27.075 | −7.106 | 1.00 | 44.37 | L | N |
| ATOM | 4655 | CA | HIS | L | 189 | −19.377 | −27.954 | −6.798 | 1.00 | 40.57 | L | C |
| ATOM | 4656 | CB | HIS | L | 189 | −18.062 | −27.277 | −7.209 | 1.00 | 42.83 | L | C |
| ATOM | 4657 | CG | HIS | L | 189 | −18.102 | −26.626 | −8.561 | 1.00 | 43.42 | L | C |
| ATOM | 4658 | CD2 | HIS | L | 189 | −18.165 | −25.320 | −8.920 | 1.00 | 44.99 | L | C |
| ATOM | 4659 | ND1 | HIS | L | 189 | −18.080 | −27.346 | −9.739 | 1.00 | 47.21 | L | N |
| ATOM | 4660 | CE1 | HIS | L | 189 | −18.125 | −26.509 | −10.763 | 1.00 | 47.77 | L | C |
| ATOM | 4661 | NE2 | HIS | L | 189 | −18.177 | −25.274 | −10.293 | 1.00 | 41.90 | L | N |
| ATOM | 4662 | C | HIS | L | 189 | −19.346 | −28.281 | −5.304 | 1.00 | 39.85 | L | C |
| ATOM | 4663 | O | HIS | L | 189 | −19.881 | −27.527 | −4.493 | 1.00 | 33.18 | L | O |
| ATOM | 4664 | N | LYS | L | 190 | −18.702 | −29.386 | −4.933 | 1.00 | 41.66 | L | N |
| ATOM | 4665 | CA | LYS | L | 190 | −18.658 | −29.788 | −3.523 | 1.00 | 44.09 | L | C |
| ATOM | 4666 | CB | LYS | L | 190 | −19.116 | −31.249 | −3.375 | 1.00 | 46.12 | L | C |
| ATOM | 4667 | CG | LYS | L | 190 | −19.364 | −31.682 | −1.908 | 1.00 | 50.60 | L | C |
| ATOM | 4668 | CD | LYS | L | 190 | −19.593 | −33.198 | −1.740 | 1.00 | 52.74 | L | C |
| ATOM | 4669 | CE | LYS | L | 190 | −18.303 | −34.023 | −1.908 | 1.00 | 51.83 | L | C |
| ATOM | 4670 | NZ | LYS | L | 190 | −18.509 | −35.513 | −1.853 | 1.00 | 47.51 | L | N |
| ATOM | 4671 | C | LYS | L | 190 | −17.337 | −29.597 | −2.760 | 1.00 | 43.80 | L | C |
| ATOM | 4672 | O | LYS | L | 190 | −17.343 | −29.150 | −1.608 | 1.00 | 40.80 | L | O |
| ATOM | 4673 | N | VAL | L | 191 | −16.223 | −29.995 | −3.372 | 1.00 | 42.44 | L | N |
| ATOM | 4674 | CA | VAL | L | 191 | −14.906 | −29.895 | −2.731 | 1.00 | 42.88 | L | C |
| ATOM | 4675 | CB | VAL | L | 191 | −13.944 | −31.025 | −3.212 | 1.00 | 44.00 | L | C |
| ATOM | 4676 | CG1 | VAL | L | 191 | −12.667 | −31.032 | −2.376 | 1.00 | 42.89 | L | C |
| ATOM | 4677 | CG2 | VAL | L | 191 | −14.631 | −32.382 | −3.126 | 1.00 | 46.02 | L | C |
| ATOM | 4678 | C | VAL | L | 191 | −14.232 | −28.535 | −2.947 | 1.00 | 43.47 | L | C |
| ATOM | 4679 | O | VAL | L | 191 | −14.008 | −28.097 | −4.087 | 1.00 | 34.72 | L | O |
| ATOM | 4680 | N | TYR | L | 192 | −13.926 | −27.866 | −1.840 | 1.00 | 36.37 | L | N |
| ATOM | 4681 | CA | TYR | L | 192 | −13.277 | −26.564 | −1.887 | 1.00 | 38.33 | L | C |
| ATOM | 4682 | CB | TYR | L | 192 | −14.178 | −25.512 | −1.246 | 1.00 | 32.59 | L | C |
| ATOM | 4683 | CG | TYR | L | 192 | −15.276 | −25.089 | −2.171 | 1.00 | 34.27 | L | C |
| ATOM | 4684 | CD1 | TYR | L | 192 | −16.423 | −25.863 | −2.327 | 1.00 | 35.13 | L | C |
| ATOM | 4685 | CE1 | TYR | L | 192 | −17.405 | −25.506 | −3.238 | 1.00 | 35.17 | L | C |
| ATOM | 4686 | CD2 | TYR | L | 192 | −15.142 | −23.949 | −2.947 | 1.00 | 31.56 | L | C |
| ATOM | 4687 | CE2 | TYR | L | 192 | −16.108 | −23.585 | −3.857 | 1.00 | 31.59 | L | C |
| ATOM | 4688 | CZ | TYR | L | 192 | −17.241 | −24.362 | −4.001 | 1.00 | 36.52 | L | C |
| ATOM | 4689 | OH | TYR | L | 192 | −18.217 | −23.967 | −4.892 | 1.00 | 39.24 | L | O |
| ATOM | 4690 | C | TYR | L | 192 | −11.927 | −26.629 | −1.193 | 1.00 | 38.61 | L | C |
| ATOM | 4691 | O | TYR | L | 192 | −11.850 | −26.988 | −0.022 | 1.00 | 38.08 | L | O |
| ATOM | 4692 | N | ALA | L | 193 | −10.868 | −26.251 | −1.904 | 1.00 | 40.03 | L | N |
| ATOM | 4693 | CA | ALA | L | 193 | −9.525 | −26.325 | −1.327 | 1.00 | 44.55 | L | C |
| ATOM | 4694 | CB | ALA | L | 193 | −8.940 | −27.702 | −1.610 | 1.00 | 47.29 | L | C |
| ATOM | 4695 | C | ALA | L | 193 | −8.503 | −25.272 | −1.733 | 1.00 | 42.21 | L | C |
| ATOM | 4696 | O | ALA | 1 | 193 | −8.527 | −24.758 | −2.853 | 1.00 | 36.92 | L | O |
| ATOM | 4697 | N | CYS | L | 194 | −7.619 | −24.936 | −0.797 | 1.00 | 38.34 | L | N |
| ATOM | 4698 | CA | CYS | L | 194 | −6.537 | −24.006 | −1.098 | 1.00 | 41.88 | L | C |
| ATOM | 4699 | C | CYS | L | 194 | −5.230 | −24.730 | −0.824 | 1.00 | 36.73 | L | C |
| ATOM | 4700 | O | CYS | L | 194 | −5.045 | −25.330 | 0.234 | 1.00 | 38.58 | L | O |
| ATOM | 4701 | CB | CYS | L | 194 | −6.624 | −22.683 | −0.322 | 1.00 | 39.58 | L | C |
| ATOM | 4702 | SG | CYS | L | 194 | −6.421 | −22.769 | 1.479 | 1.00 | 47.30 | L | S |
| ATOM | 4703 | N | GLU | L | 195 | −4.403 | −24.795 | −1.858 | 1.00 | 36.55 | L | N |
| ATOM | 4704 | CA | GLU | L | 195 | −3.093 | −25.439 | −1.798 | 1.00 | 36.80 | L | C |
| ATOM | 4705 | CB | GLU | L | 195 | −2.851 | −26.247 | −3.076 | 0.00 | 35.65 | L | C |
| ATOM | 4706 | CG | GLU | L | 195 | −1.459 | −26.850 | −3.187 | 0.00 | 35.13 | L | C |
| ATOM | 4707 | CD | GLU | L | 195 | −1.176 | −27.418 | −4.565 | 0.00 | 34.69 | L | C |
| ATOM | 4708 | OE1 | GLU | L | 195 | −1.043 | −26.624 | −5.521 | 0.00 | 34.25 | L | O |
| ATOM | 4709 | OE2 | GLU | L | 195 | −1.085 | −28.657 | −4.692 | 0.00 | 34.41 | L | O |
| ATOM | 4710 | C | GLU | L | 195 | −2.007 | −24.359 | −1.657 | 1.00 | 34.71 | L | C |
| ATOM | 4711 | O | GLU | L | 195 | −1.839 | −23.510 | −2.539 | 1.00 | 33.65 | L | O |
| ATOM | 4712 | N | VAL | L | 196 | −1.253 | −24.433 | −0.566 | 1.00 | 32.40 | L | N |
| ATOM | 4713 | CA | VAL | L | 196 | −0.191 | −23.471 | −0.284 | 1.00 | 32.65 | L | C |
| ATOM | 4714 | CB | VAL | L | 196 | −0.294 | −22.968 | 1.165 | 1.00 | 26.52 | L | C |
| ATOM | 4715 | CG1 | VAL | L | 196 | 0.773 | −21.952 | 1.441 | 1.00 | 32.60 | L | C |
| ATOM | 4716 | CG2 | VAL | L | 196 | −1.665 | −22.372 | 1.410 | 1.00 | 29.07 | L | C |
| ATOM | 4717 | C | VAL | L | 196 | 1.220 | −24.027 | −0.525 | 1.00 | 35.95 | L | C |
| ATOM | 4718 | O | VAL | L | 196 | 1.585 | −25.080 | −0.001 | 1.00 | 39.88 | L | O |
| ATOM | 4719 | N | THR | L | 197 | 1.990 | −23.323 | −1.348 | 1.00 | 38.44 | L | N |
| ATOM | 4720 | CA | THR | L | 197 | 3.367 | −23.701 | −1.653 | 1.00 | 41.46 | L | C |
| ATOM | 4721 | CB | THR | L | 197 | 3.637 | −23.734 | −3.178 | 1.00 | 43.10 | L | C |
| ATOM | 4722 | OG1 | THR | L | 197 | 2.940 | −24.832 | −3.767 | 1.00 | 50.60 | L | O |
| ATOM | 4723 | CG2 | THR | L | 197 | 5.113 | −23.908 | −3.458 | 1.00 | 45.02 | L | C |
| ATOM | 4724 | C | THR | L | 197 | 4.283 | −22.651 | −1.022 | 1.00 | 43.25 | L | C |
| ATOM | 4725 | O | THR | L | 197 | 4.165 | −21.455 | −1.315 | 1.00 | 45.12 | L | O |
| ATOM | 4726 | N | HIS | L | 198 | 5.207 | −23.105 | −0.182 | 1.00 | 40.13 | L | N |
| ATOM | 4727 | CA | HIS | L | 198 | 6.140 | −22.217 | 0.506 | 1.00 | 33.25 | L | C |
| ATOM | 4728 | CB | HIS | L | 198 | 5.476 | −21.688 | 1.782 | 1.00 | 30.16 | L | C |
| ATOM | 4729 | CG | HIS | L | 198 | 6.283 | −20.668 | 2.515 | 1.00 | 28.81 | L | C |
| ATOM | 4730 | CD2 | HIS | L | 198 | 6.363 | −19.325 | 2.370 | 1.00 | 27.25 | L | C |
| ATOM | 4731 | ND1 | HIS | L | 198 | 7.137 | −20.996 | 3.549 | 1.00 | 26.04 | L | N |
| ATOM | 4732 | CE1 | HIS | L | 198 | 7.710 | −19.897 | 4.007 | 1.00 | 29.27 | L | C |
| ATOM | 4733 | NE2 | HIS | L | 198 | 7.258 | −18.869 | 3.307 | 1.00 | 32.61 | L | N |
| ATOM | 4734 | C | HIS | L | 198 | 7.423 | −22.979 | 0.844 | 1.00 | 35.26 | L | C |
| ATOM | 4735 | O | HIS | L | 198 | 7.392 | −24.183 | 1.140 | 1.00 | 31.17 | L | O |
| ATOM | 4736 | N | GLN | L | 199 | 8.541 | −22.255 | 0.827 | 1.00 | 39.62 | L | N |
| ATOM | 4737 | CA | GLN | L | 199 | 9.872 | −22.800 | 1.111 | 1.00 | 40.31 | L | C |
| ATOM | 4738 | CB | GLN | L | 199 | 10.881 | −21.648 | 1.138 | 1.00 | 43.33 | L | C |
| ATOM | 4739 | CG | GLN | L | 199 | 12.341 | −22.069 | 1.242 | 1.00 | 47.52 | L | C |
| ATOM | 4740 | CD | GLN | L | 199 | 13.278 | −20.889 | 1.442 | 1.00 | 47.36 | L | C |
| ATOM | 4741 | OE1 | GLN | L | 199 | 14.350 | −21.034 | 2.031 | 1.00 | 52.10 | L | O |
| ATOM | 4742 | NE2 | GLN | L | 199 | 12.873 | −19.712 | 0.965 | 1.00 | 42.82 | L | N |
| ATOM | 4743 | C | GLN | L | 199 | 9.949 | −23.598 | 2.420 | 1.00 | 40.78 | L | C |
| ATOM | 4744 | O | GLN | L | 199 | 10.683 | −24.586 | 2.516 | 1.00 | 40.93 | L | O |
| ATOM | 4745 | N | GLY | L | 200 | 9.159 | −23.184 | 3.408 | 1.00 | 40.71 | L | N |
| ATOM | 4746 | CA | GLY | L | 200 | 9.141 | −23.857 | 4.699 | 1.00 | 43.25 | L | C |
| ATOM | 4749 | N | LEU | L | 201 | 7.845 | −25.674 | 3.667 | 1.00 | 43.11 | L | N |
| ATOM | 4751 | CA | LEU | L | 201 | 7.132 | −26.947 | 3.684 | 1.00 | 44.48 | L | C |
| ATOM | 4752 | CB | LEU | L | 201 | 5.631 | −26.715 | 3.499 | 1.00 | 44.82 | L | C |
| ATOM | 4753 | CG | LEU | L | 201 | 5.003 | −25.614 | 4.359 | 1.00 | 41.50 | L | C |
| ATOM | 4754 | CD1 | LEU | L | 201 | 3.824 | −24.994 | 3.626 | 1.00 | 43.37 | L | N |
| ATOM | 4755 | CD2 | LEU | L | 201 | 4.564 | −26.188 | 5.695 | 1.00 | 44.22 | L | N |
| ATOM | 4756 | C | LEU | L | 201 | 7.650 | −27.878 | 2.593 | 1.00 | 44.76 | L | C |
| ATOM | 4757 | O | LEU | L | 201 | 7.859 | −27.471 | 1.447 | 1.00 | 43.60 | L | O |
| ATOM | 4758 | N | SER | L | 202 | 7.852 | −29.150 | 2.981 | 1.00 | 42.46 | L | N |
| ATOM | 4759 | CA | SER | L | 202 | 8.372 | −30.209 | 2.103 | 1.00 | 45.23 | L | C |
| ATOM | 4760 | CB | SER | L | 202 | 8.147 | −31.575 | 2.757 | 1.00 | 48.00 | L | C |
| ATOM | 4761 | OG | SER | L | 202 | 8.642 | −31.585 | 4.079 | 1.00 | 54.29 | L | O |
| ATOM | 4762 | C | SER | L | 202 | 7.728 | −30.221 | 0.724 | 1.00 | 42.16 | L | C |
| ATOM | 4763 | O | SER | L | 202 | 8.402 | −30.167 | −0.305 | 1.00 | 39.94 | L | O |
| ATOM | 4764 | N | SER | L | 203 | 6.410 | −30.350 | 0.735 | 1.00 | 42.29 | L | N |
| ATOM | 4765 | CA | SER | L | 203 | 5.591 | −30.372 | −0.471 | 1.00 | 45.07 | L | C |
| ATOM | 4766 | CB | SER | L | 203 | 5.242 | −31.817 | −0.859 | 1.00 | 44.31 | L | C |
| ATOM | 4767 | OG | SER | L | 203 | 4.667 | −32.512 | 0.238 | 1.00 | 42.52 | L | O |
| ATOM | 4768 | C | SER | L | 203 | 4.334 | −29.595 | −0.101 | 1.00 | 44.27 | L | C |
| ATOM | 4769 | O | SER | L | 203 | 3.981 | −29.499 | 1.077 | 1.00 | 46.09 | L | O |
| ATOM | 4770 | N | PRO | L | 204 | 3.623 | −29.066 | −1.102 | 1.00 | 44.89 | L | N |
| ATOM | 4771 | CD | PRO | L | 204 | 3.903 | −29.280 | −2.534 | 1.00 | 44.74 | L | C |
| ATOM | 4772 | CA | PRO | L | 204 | 2.399 | −28.284 | −0.931 | 1.00 | 43.04 | L | C |
| ATOM | 4773 | CB | PRO | L | 204 | 1.802 | −28.312 | −2.329 | 1.00 | 44.84 | L | C |
| ATOM | 4774 | CG | PRO | L | 204 | 3.015 | −28.244 | −3.188 | 1.00 | 45.91 | L | C |
| ATOM | 4775 | C | PRO | L | 204 | 1.403 | −28.786 | 0.108 | 1.00 | 45.29 | L | C |
| ATOM | 4776 | O | PRO | L | 204 | 1.090 | −29.972 | 0.156 | 1.00 | 49.17 | L | O |
| ATOM | 4777 | N | VAL | L | 205 | 0.948 | −27.873 | 0.963 | 1.00 | 41.64 | L | N |
| ATOM | 4778 | CA | VAL | L | 205 | −0.039 | −28.175 | 1.995 | 1.00 | 40.89 | L | C |
| ATOM | 4779 | CB | VAL | L | 205 | 0.206 | −27.327 | 3.266 | 1.00 | 39.58 | L | C |
| ATOM | 4780 | CG1 | VAL | L | 205 | −1.022 | −27.321 | 4.157 | 1.00 | 36.28 | L | C |
| ATOM | 4781 | CG2 | VAL | L | 205 | 1.363 | −27.887 | 4.029 | 1.00 | 37.41 | L | C |
| ATOM | 4782 | C | VAL | L | 205 | −1.417 | −27.841 | 1.416 | 1.00 | 43.46 | L | C |
| ATOM | 4783 | O | VAL | L | 205 | −1.597 | −26.784 | 0.807 | 1.00 | 46.29 | L | O |
| ATOM | 4784 | N | THR | L | 206 | −2.381 | −28.743 | 1.596 | 1.00 | 45.00 | L | N |
| ATOM | 4785 | CA | THR | L | 206 | −3.733 | −28.532 | 1.073 | 1.00 | 40.96 | L | C |
| ATOM | 4786 | CB | THR | L | 206 | −4.109 | −29.587 | −0.014 | 1.00 | 40.46 | L | C |
| ATOM | 4787 | OG1 | THR | L | 206 | −3.423 | −29.295 | −1.236 | 1.00 | 40.96 | L | O |
| ATOM | 4788 | CG2 | THR | L | 206 | −5.610 | −29.572 | −0.293 | 1.00 | 41.69 | L | C |
| ATOM | 4789 | C | THR | L | 206 | −4.816 | −28.571 | 2.136 | 1.00 | 38.56 | L | C |
| ATOM | 4790 | O | THR | L | 206 | −4.945 | −29.559 | 2.857 | 1.00 | 40.82 | L | O |
| ATOM | 4791 | N | LYS | L | 207 | −5.587 | −27.492 | 2.232 | 1.00 | 36.34 | L | N |
| ATOM | 4792 | CA | LYS | L | 207 | −6.702 | −27.446 | 3.171 | 1.00 | 37.89 | L | C |
| ATOM | 4793 | CB | LYS | L | 207 | −6.597 | −26.243 | 4.106 | 1.00 | 37.28 | L | C |
| ATOM | 4794 | CG | LYS | L | 207 | −5.619 | −26.449 | 5.267 | 1.00 | 41.03 | L | C |
| ATOM | 4795 | CD | LYS | L | 207 | −6.136 | −27.486 | 6.258 | 1.00 | 43.76 | L | C |
| ATOM | 4796 | CE | LYS | L | 207 | −5.228 | −27.646 | 7.480 | 1.00 | 48.44 | L | C |
| ATOM | 4797 | NZ | LYS | L | 207 | −3.863 | −28.142 | 7.136 | 1.00 | 48.48 | L | N |
| ATOM | 4798 | C | LYS | L | 207 | −8.002 | −27.424 | 2.363 | 1.00 | 36.14 | L | C |
| ATOM | 4799 | O | LYS | L | 207 | −8.126 | −26.692 | 1.375 | 1.00 | 34.12 | L | O |
| ATOM | 4800 | N | SER | L | 208 | −8.957 | −28.253 | 2.770 | 1.00 | 38.77 | L | N |
| ATOM | 4801 | CA | SER | L | 208 | −10.230 | −28.353 | 2.068 | 1.00 | 41.13 | L | C |
| ATOM | 4802 | CB | SER | L | 208 | −10.114 | −29.365 | 0.923 | 1.00 | 43.01 | L | C |
| ATOM | 4803 | OG | SER | L | 208 | −9.670 | −30.624 | 1.392 | 1.00 | 48.14 | L | O |
| ATOM | 4804 | C | SER | L | 208 | −11.399 | −28.744 | 2.953 | 1.00 | 40.77 | L | C |
| ATOM | 4805 | O | SER | L | 208 | −11.215 | −29.146 | 4.097 | 1.00 | 44.22 | L | O |
| ATOM | 4806 | N | PHE | L | 209 | −12.605 | −28.625 | 2.396 | 1.00 | 43.97 | L | N |
| ATOM | 4807 | CA | PHE | L | 209 | −13.845 | −28.980 | 3.083 | 1.00 | 45.09 | L | C |
| ATOM | 4808 | CB | PHE | L | 209 | −14.332 | −27.824 | 3.979 | 1.00 | 44.68 | L | C |
| ATOM | 4809 | CG | PHE | L | 209 | −14.779 | −26.607 | 3.217 | 1.00 | 46.54 | L | C |
| ATOM | 4810 | CD1 | PHE | L | 209 | −16.095 | −26.490 | 2.768 | 1.00 | 48.38 | L | C |
| ATOM | 4811 | CD2 | PHE | L | 209 | −13.880 | −25.592 | 2.913 | 1.00 | 46.50 | L | C |
| ATOM | 4812 | CE1 | PHE | L | 209 | −16.505 | −25.379 | 2.024 | 1.00 | 47.41 | L | C |
| ATOM | 4813 | CE2 | PHE | L | 209 | −14.276 | −24.482 | 2.173 | 1.00 | 42.57 | L | C |
| ATOM | 4814 | CZ | PHE | L | 209 | −15.591 | −24.375 | 1.725 | 1.00 | 47.38 | L | C |
| ATOM | 4815 | C | PHE | L | 209 | −14.908 | −29.322 | 2.030 | 1.00 | 46.82 | L | C |
| ATOM | 4816 | O | PHE | L | 209 | −14.914 | −28.740 | 0.941 | 1.00 | 48.02 | L | O |
| ATOM | 4817 | N | ASN | L | 210 | −15.761 | −30.304 | 2.332 | 1.00 | 51.05 | L | N |
| ATOM | 4818 | CA | ASN | L | 210 | −16.841 | −30.712 | 1.426 | 1.00 | 46.83 | L | C |
| ATOM | 4819 | CB | ASN | L | 210 | −17.302 | −32.145 | 1.728 | 1.00 | 52.02 | L | C |
| ATOM | 4820 | CG | ASN | L | 210 | −16.325 | −33.202 | 1.216 | 1.00 | 55.46 | L | C |
| ATOM | 4821 | OD1 | ASN | L | 210 | −16.236 | −34.309 | 1.768 | 1.00 | 57.51 | L | O |
| ATOM | 4822 | ND2 | ASN | L | 210 | −15.600 | −32.872 | 0.143 | 1.00 | 57.94 | L | N |
| ATOM | 4823 | C | ASN | L | 210 | −17.972 | −29.738 | 1.681 | 1.00 | 44.00 | L | C |
| ATOM | 4824 | O | ASN | L | 210 | −18.354 | −29.539 | 2.829 | 1.00 | 37.80 | L | O |
| ATOM | 4825 | N | ARG | L | 211 | −18.498 | −29.122 | 0.624 | 1.00 | 43.68 | L | N |
| ATOM | 4826 | CA | ARG | L | 211 | −19.567 | −28.129 | 0.775 | 1.00 | 47.79 | L | C |
| ATOM | 4827 | CB | ARG | L | 211 | −20.053 | −27.625 | −0.583 | 1.00 | 48.31 | L | C |
| ATOM | 4828 | CG | ARG | L | 211 | −21.142 | −26.570 | −0.472 | 1.00 | 44.44 | L | C |
| ATOM | 4829 | CD | ARG | L | 211 | −21.492 | −25.995 | −1.822 | 1.00 | 44.64 | L | C |
| ATOM | 4830 | NE | ARG | L | 211 | −22.026 | −27.010 | −2.717 | 1.00 | 46.38 | L | N |
| ATOM | 4831 | CZ | ARG | L | 211 | −23.203 | −27.601 | −2.551 | 1.00 | 47.06 | L | C |
| ATOM | 4832 | NH1 | ARG | L | 211 | −23.972 | −27.270 | −1.516 | 1.00 | 43.94 | L | N |
| ATOM | 4833 | NH2 | ARG | L | 211 | −23.596 | −28.535 | −3.406 | 1.00 | 46.43 | L | N |
| ATOM | 4834 | C | ARG | L | 211 | −20.757 | −28.562 | 1.625 | 1.00 | 49.68 | L | C |
| ATOM | 4835 | O | ARG | L | 211 | −21.276 | −29.677 | 1.481 | 1.00 | 50.79 | L | O |
| ATOM | 4836 | N | GLY | L | 212 | −21.185 | −27.655 | 2.500 | 1.00 | 53.40 | L | N |
| ATOM | 4837 | CA | GLY | L | 212 | −22.301 | −27.928 | 3.389 | 1.00 | 62.42 | L | C |
| ATOM | 4838 | C | GLY | L | 212 | −21.953 | −28.861 | 4.539 | 1.00 | 66.95 | L | C |
| ATOM | 4839 | O | GLY | L | 212 | −21.928 | −28.450 | 5.705 | 1.00 | 68.74 | L | O |
| ATOM | 4840 | N | GLU | L | 213 | −21.638 | −30.110 | 4.197 | 1.00 | 70.66 | L | N |
| ATOM | 4841 | CA | GLU | L | 213 | −21.304 | −31.149 | 5.174 | 1.00 | 70.59 | L | C |
| ATOM | 4842 | CB | GLU | L | 213 | −21.858 | −32.500 | 4.692 | 1.00 | 71.54 | L | C |
| ATOM | 4843 | CG | GLU | L | 213 | −23.390 | −32.491 | 4.500 | 1.00 | 73.78 | L | C |
| ATOM | 4844 | CD | GLU | L | 213 | −23.949 | −33.766 | 3.871 | 1.00 | 74.90 | L | C |
| ATOM | 4845 | OE1 | GLU | L | 213 | −23.500 | −34.873 | 4.260 | 1.00 | 72.55 | L | O |
| ATOM | 4846 | OE2 | GLU | L | 213 | −24.849 | −33.653 | 2.998 | 1.00 | 70.14 | L | O |
| ATOM | 4847 | C | GLU | L | 213 | −19.813 | −31.258 | 5.485 | 1.00 | 65.74 | L | C |
| ATOM | 4848 | O | GLU | L | 213 | −19.378 | −30.918 | 6.588 | 1.00 | 65.21 | L | O |
| ATOM | 4849 | N | ASP | M | 1 | 27.686 | 16.369 | 47.861 | 1.00 | 57.92 | M | N |
| ATOM | 4850 | CA | ASP | M | 1 | 26.996 | 15.892 | 46.671 | 1.00 | 60.41 | M | C |
| ATOM | 4851 | CB | ASP | M | 1 | 26.980 | 16.948 | 45.528 | 1.00 | 65.46 | M | C |
| ATOM | 4852 | CG | ASP | M | 1 | 28.245 | 17.816 | 45.455 | 1.00 | 70.57 | M | C |
| ATOM | 4853 | OD1 | ASP | M | 1 | 29.271 | 17.357 | 44.894 | 1.00 | 73.95 | M | O |
| ATOM | 4854 | OD2 | ASP | M | 1 | 28.188 | 18.989 | 45.902 | 1.00 | 70.80 | M | O |
| ATOM | 4855 | C | ASP | M | 1 | 27.452 | 14.510 | 46.181 | 1.00 | 55.91 | M | C |
| ATOM | 4856 | O | ASP | M | 1 | 28.409 | 13.939 | 46.707 | 1.00 | 58.24 | M | O |
| ATOM | 4857 | N | ILE | M | 2 | 26.695 | 13.943 | 45.243 | 1.00 | 48.05 | M | N |
| ATOM | 4858 | CA | ILE | M | 2 | 26.989 | 12.628 | 44.672 | 1.00 | 40.29 | M | C |
| ATOM | 4859 | CB | ILE | M | 2 | 25.726 | 12.004 | 44.022 | 1.00 | 33.75 | M | C |
| ATOM | 4860 | CG2 | ILE | M | 2 | 26.083 | 10.794 | 43.216 | 1.00 | 24.88 | M | C |
| ATOM | 4861 | CG1 | ILE | M | 2 | 24.725 | 11.606 | 45.105 | 1.00 | 31.24 | M | C |
| ATOM | 4862 | CD1 | ILE | M | 2 | 23.397 | 11.107 | 44.556 | 1.00 | 31.91 | M | C |
| ATOM | 4863 | C | ILE | M | 2 | 28.093 | 12.737 | 43.635 | 1.00 | 38.98 | M | C |
| ATOM | 4864 | O | ILE | M | 2 | 28.048 | 13.588 | 42.734 | 1.00 | 40.06 | M | O |
| ATOM | 4865 | N | GLN | M | 3 | 29.078 | 11.859 | 43.767 | 1.00 | 37.43 | M | N |
| ATOM | 4866 | CA | GLN | M | 3 | 30.217 | 11.840 | 42.861 | 1.00 | 38.34 | M | C |
| ATOM | 4867 | CB | GLN | M | 3 | 31.476 | 11.374 | 43.621 | 1.00 | 45.04 | M | C |
| ATOM | 4868 | CG | GLN | M | 3 | 31.574 | 11.807 | 45.103 | 1.00 | 49.04 | M | C |
| ATOM | 4869 | CD | GLN | M | 3 | 32.189 | 13.185 | 45.285 | 1.00 | 53.31 | M | C |
| ATOM | 4870 | OE1 | GLN | M | 3 | 33.389 | 13.306 | 45.566 | 1.00 | 54.86 | M | O |
| ATOM | 4871 | NE2 | GLN | M | 3 | 31.372 | 14.232 | 45.138 | 1.00 | 52.39 | M | N |
| ATOM | 4872 | C | GLN | M | 3 | 29.960 | 10.876 | 41.703 | 1.00 | 34.71 | M | C |
| ATOM | 4873 | O | GLN | M | 3 | 29.665 | 9.704 | 41.923 | 1.00 | 32.54 | M | O |
| ATOM | 4874 | N | MET | M | 4 | 30.064 | 11.365 | 40.474 | 1.00 | 33.72 | M | N |
| ATOM | 4875 | CA | MET | M | 4 | 29.888 | 10.504 | 39.315 | 1.00 | 28.82 | M | C |
| ATOM | 4876 | CB | MET | M | 4 | 28.891 | 11.125 | 38.329 | 1.00 | 31.51 | M | C |
| ATOM | 4877 | CG | MET | M | 4 | 27.448 | 11.195 | 38.813 | 1.00 | 29.80 | M | C |
| ATOM | 4878 | SD | MET | M | 4 | 26.747 | 9.583 | 39.227 | 1.00 | 37.38 | M | S |
| ATOM | 4879 | CE | MET | M | 4 | 26.048 | 9.144 | 37.724 | 1.00 | 30.21 | M | C |
| ATOM | 4880 | C | MET | M | 4 | 31.251 | 10.346 | 38.636 | 1.00 | 30.45 | M | C |
| ATOM | 4881 | O | MET | M | 4 | 31.800 | 11.320 | 38.145 | 1.00 | 36.05 | M | O |
| ATOM | 4882 | N | THR | M | 5 | 31.819 | 9.142 | 38.644 | 1.00 | 27.60 | M | N |
| ATOM | 4883 | CA | THR | M | 5 | 33.106 | 8.889 | 37.980 | 1.00 | 23.48 | M | C |
| ATOM | 4884 | CB | THR | M | 5 | 33.999 | 7.918 | 38.794 | 1.00 | 25.01 | M | C |
| ATOM | 4885 | OG1 | THR | M | 5 | 34.102 | 8.363 | 40.149 | 1.00 | 29.51 | M | O |
| ATOM | 4886 | CG2 | THR | M | 5 | 35.393 | 7.842 | 38.164 | 1.00 | 17.68 | M | C |
| ATOM | 4887 | C | THR | M | 5 | 32.854 | 8.202 | 36.618 | 1.00 | 24.67 | M | C |
| ATOM | 4888 | O | THR | M | 5 | 32.374 | 7.071 | 36.568 | 1.00 | 21.84 | M | O |
| ATOM | 4889 | N | GLN | M | 6 | 33.268 | 8.837 | 35.533 | 1.00 | 18.26 | M | N |
| ATOM | 4890 | CA | GLN | M | 6 | 33.047 | 8.287 | 34.215 | 1.00 | 20.95 | M | C |
| ATOM | 4891 | CB | GLN | M | 6 | 32.527 | 9.426 | 33.345 | 1.00 | 20.29 | M | C |
| ATOM | 4892 | CG | GLN | M | 6 | 31.976 | 9.038 | 32.023 | 1.00 | 18.13 | M | C |
| ATOM | 4893 | CD | GLN | M | 6 | 31.292 | 10.198 | 31.351 | 1.00 | 23.46 | M | C |
| ATOM | 4894 | OE1 | GLN | M | 6 | 30.729 | 11.081 | 32.013 | 1.00 | 22.84 | M | O |
| ATOM | 4895 | NE2 | GLN | M | 6 | 31.332 | 10.210 | 30.022 | 1.00 | 26.79 | M | N |
| ATOM | 4896 | C | GLN | M | 6 | 34.339 | 7.678 | 33.677 | 1.00 | 24.68 | M | C |
| ATOM | 4897 | O | GLN | M | 6 | 35.404 | 8.271 | 33.808 | 1.00 | 28.34 | M | O |
| ATOM | 4898 | N | SER | M | 7 | 34.243 | 6.498 | 33.072 | 1.00 | 28.56 | M | N |
| ATOM | 4899 | CA | SER | M | 7 | 35.414 | 5.813 | 32.520 | 1.00 | 32.77 | M | C |
| ATOM | 4900 | CB | SER | M | 7 | 35.769 | 4.600 | 33.375 | 1.00 | 29.63 | M | C |
| ATOM | 4901 | OG | SER | M | 7 | 35.995 | 4.983 | 34.708 | 1.00 | 43.98 | M | O |
| ATOM | 4902 | C | SER | M | 7 | 35.166 | 5.326 | 31.101 | 1.00 | 31.23 | M | C |
| ATOM | 4903 | O | SER | M | 7 | 34.080 | 4.842 | 30.798 | 1.00 | 35.30 | M | O |
| ATOM | 4904 | N | PRO | M | 8 | 36.162 | 5.472 | 30.204 | 1.00 | 31.11 | M | N |
| ATOM | 4905 | CD | PRO | M | 8 | 36.131 | 4.877 | 28.850 | 1.00 | 24.27 | M | C |
| ATOM | 4906 | CA | PRO | M | 8 | 37.472 | 6.075 | 30.476 | 1.00 | 25.60 | M | C |
| ATOM | 4907 | CB | PRO | M | 8 | 38.363 | 5.392 | 29.444 | 1.00 | 23.47 | M | C |
| ATOM | 4908 | CG | PRO | M | 8 | 37.450 | 5.341 | 28.250 | 1.00 | 26.58 | M | C |
| ATOM | 4909 | C | PRO | M | 8 | 37.331 | 7.558 | 30.165 | 1.00 | 27.29 | M | C |
| ATOM | 4910 | O | PRO | M | 8 | 36.266 | 7.993 | 29.740 | 1.00 | 27.35 | M | O |
| ATOM | 4911 | N | SER | M | 9 | 38.387 | 8.334 | 30.368 | 1.00 | 27.96 | M | N |
| ATOM | 4912 | CA | SER | M | 9 | 38.329 | 9.756 | 30.061 | 1.00 | 26.77 | M | C |
| ATOM | 4913 | CB | SER | M | 9 | 39.464 | 10.508 | 30.759 | 1.00 | 33.76 | M | C |
| ATOM | 4914 | OG | SER | M | 9 | 39.162 | 10.611 | 32.145 | 1.00 | 44.13 | M | O |
| ATOM | 4915 | C | SER | M | 9 | 38.324 | 10.000 | 28.553 | 1.00 | 21.06 | M | C |
| ATOM | 4916 | O | SER | M | 9 | 37.792 | 10.998 | 28.077 | 1.00 | 16.15 | M | O |
| ATOM | 4917 | N | SER | M | 10 | 38.934 | 9.090 | 27.802 | 1.00 | 20.10 | M | N |
| ATOM | 4918 | CA | SER | M | 10 | 38.931 | 9.186 | 26.348 | 1.00 | 24.89 | M | C |
| ATOM | 4919 | CB | SER | M | 10 | 40.010 | 10.133 | 25.826 | 1.00 | 24.70 | M | C |
| ATOM | 4920 | OG | SER | M | 10 | 41.265 | 9.493 | 25.813 | 1.00 | 36.48 | M | O |
| ATOM | 4921 | C | SER | M | 10 | 39.145 | 7.798 | 25.783 | 1.00 | 23.53 | M | C |
| ATOM | 4922 | O | SER | M | 10 | 39.770 | 6.958 | 26.416 | 1.00 | 23.80 | M | O |
| ATOM | 4923 | N | LEU | M | 11 | 38.547 | 7.516 | 24.636 | 1.00 | 26.32 | M | N |
| ATOM | 4924 | CA | LEU | M | 11 | 38.749 | 6.207 | 24.030 | 1.00 | 29.50 | M | C |
| ATOM | 4925 | CB | LEU | M | 11 | 37.669 | 5.203 | 24.447 | 1.00 | 27.40 | M | C |
| ATOM | 4926 | CG | LEU | M | 11 | 36.223 | 5.417 | 24.050 | 1.00 | 31.65 | M | C |
| ATOM | 4927 | CD1 | LEU | M | 11 | 35.916 | 4.673 | 22.749 | 1.00 | 35.55 | M | C |
| ATOM | 4928 | CD2 | LEU | M | 11 | 35.366 | 4.877 | 25.164 | 1.00 | 32.50 | M | C |
| ATOM | 4929 | C | LEU | M | 11 | 38.904 | 6.297 | 22.524 | 1.00 | 27.05 | M | C |
| ATOM | 4930 | O | LEU | M | 11 | 38.343 | 7.179 | 21.875 | 1.00 | 29.40 | M | O |
| ATOM | 4931 | N | SER | M | 12 | 39.778 | 5.450 | 21.996 | 1.00 | 31.18 | M | N |
| ATOM | 4932 | CA | SER | M | 12 | 40.049 | 5.418 | 20.563 | 1.00 | 26.99 | M | C |
| ATOM | 4933 | CB | SER | M | 12 | 41.540 | 5.183 | 20.301 | 1.00 | 29.74 | M | C |
| ATOM | 4934 | OG | SER | M | 12 | 42.334 | 6.196 | 20.888 | 1.00 | 30.04 | M | O |
| ATOM | 4935 | C | SER | M | 12 | 39.246 | 4.300 | 19.934 | 1.00 | 24.17 | M | C |
| ATOM | 4936 | O | SER | M | 12 | 39.289 | 3.149 | 20.376 | 1.00 | 19.25 | M | O |
| ATOM | 4937 | N | ALA | M | 13 | 38.538 | 4.634 | 18.873 | 1.00 | 19.89 | M | N |
| ATOM | 4938 | CA | ALA | M | 13 | 37.725 | 3.645 | 18.207 | 1.00 | 17.11 | M | C |
| ATOM | 4939 | CB | ALA | M | 13 | 36.329 | 3.636 | 18.809 | 1.00 | 22.65 | M | C |
| ATOM | 4940 | C | ALA | M | 13 | 37.663 | 3.857 | 16.704 | 1.00 | 20.45 | M | C |
| ATOM | 4941 | O | ALA | M | 13 | 38.029 | 4.916 | 16.185 | 1.00 | 23.13 | M | O |
| ATOM | 4942 | N | SER | M | 14 | 37.227 | 2.819 | 16.002 | 1.00 | 21.15 | M | N |
| ATOM | 4943 | CA | SER | M | 14 | 37.110 | 2.873 | 14.548 | 1.00 | 26.12 | M | C |
| ATOM | 4944 | CB | SER | M | 14 | 37.871 | 1.710 | 13.918 | 1.00 | 22.52 | M | C |
| ATOM | 4945 | OG | SER | M | 14 | 39.125 | 1.548 | 14.545 | 1.00 | 25.44 | M | O |
| ATOM | 4946 | C | SER | M | 14 | 35.647 | 2.758 | 14.178 | 1.00 | 21.63 | M | C |
| ATOM | 4947 | O | SER | M | 14 | 34.844 | 2.273 | 14.967 | 1.00 | 22.91 | M | O |
| ATOM | 4948 | N | VAL | M | 15 | 35.298 | 3.216 | 12.982 | 1.00 | 21.98 | M | N |
| ATOM | 4949 | CA | VAL | M | 15 | 33.923 | 3.122 | 12.511 | 1.00 | 15.32 | M | C |
| ATOM | 4950 | CB | VAL | M | 15 | 33.799 | 3.668 | 11.073 | 1.00 | 18.38 | M | C |
| ATOM | 4951 | CG1 | VAL | M | 15 | 32.453 | 3.260 | 10.435 | 1.00 | 11.57 | M | C |
| ATOM | 4952 | CG2 | VAL | M | 15 | 33.936 | 5.187 | 11.095 | 1.00 | 14.07 | M | C |
| ATOM | 4953 | C | VAL | M | 15 | 33.522 | 1.647 | 12.541 | 1.00 | 18.21 | M | C |
| ATOM | 4954 | O | VAL | M | 15 | 34.309 | 0.776 | 12.168 | 1.00 | 16.49 | M | O |
| ATOM | 4955 | N | GLY | M | 16 | 32.330 | 1.371 | 13.059 | 1.00 | 17.34 | M | N |
| ATOM | 4956 | CA | GLY | M | 16 | 31.844 | 0.004 | 13.137 | 1.00 | 12.45 | M | C |
| ATOM | 4957 | C | GLY | M | 16 | 32.045 | −0.677 | 14.479 | 1.00 | 20.95 | M | C |
| ATOM | 4958 | O | GLY | M | 16 | 31.394 | −1.673 | 14.775 | 1.00 | 26.06 | M | O |
| ATOM | 4959 | N | ASP | M | 17 | 32.945 | −0.148 | 15.298 | 1.00 | 23.36 | M | N |
| ATOM | 4960 | CA | ASP | M | 17 | 33.232 | −0.736 | 16.603 | 1.00 | 21.26 | M | C |
| ATOM | 4961 | CB | ASP | M | 17 | 34.408 | −0.011 | 17.273 | 1.00 | 27.06 | M | C |
| ATOM | 4962 | CG | ASP | M | 17 | 35.762 | −0.432 | 16.738 | 1.00 | 31.55 | M | C |
| ATOM | 4963 | OD1 | ASP | M | 17 | 35.845 | −1.307 | 15.837 | 1.00 | 30.24 | M | O |
| ATOM | 4964 | OD2 | ASP | M | 17 | 36.757 | 0.117 | 17.257 | 1.00 | 31.09 | M | O |
| ATOM | 4965 | C | ASP | M | 17 | 32.065 | −0.647 | 17.557 | 1.00 | 24.00 | M | C |
| ATOM | 4966 | O | ASP | M | 17 | 31.175 | 0.200 | 17.416 | 1.00 | 26.25 | M | O |
| ATOM | 4967 | N | ARG | M | 18 | 32.077 | −1.535 | 18.537 | 1.00 | 23.84 | M | N |
| ATOM | 4968 | CA | ARG | M | 18 | 31.067 | −1.513 | 19.569 | 1.00 | 28.80 | M | C |
| ATOM | 4969 | CB | ARG | M | 18 | 30.600 | −2.915 | 19.927 | 1.00 | 23.46 | M | C |
| ATOM | 4970 | CG | ARG | M | 18 | 29.416 | −2.900 | 20.893 | 1.00 | 34.77 | M | C |
| ATOM | 4971 | CD | ARG | M | 18 | 28.736 | −4.263 | 21.025 | 1.00 | 42.40 | M | C |
| ATOM | 4972 | NE | ARG | M | 18 | 27.900 | −4.337 | 22.224 | 1.00 | 48.06 | M | N |
| ATOM | 4973 | CZ | ARG | M | 18 | 28.379 | −4.430 | 23.465 | 1.00 | 51.19 | M | C |
| ATOM | 4974 | NH1 | ARG | M | 18 | 29.694 | −4.468 | 23.677 | 1.00 | 50.66 | M | N |
| ATOM | 4975 | NH2 | ARG | M | 18 | 27.544 | −4.460 | 24.500 | 1.00 | 49.13 | M | N |
| ATOM | 4976 | C | ARG | M | 18 | 31.787 | −0.848 | 20.748 | 1.00 | 31.77 | M | C |
| ATOM | 4977 | O | ARG | M | 18 | 32.826 | −1.341 | 21.193 | 1.00 | 30.84 | M | O |
| ATOM | 4978 | N | VAL | M | 19 | 31.312 | 0.327 | 21.164 | 1.00 | 27.71 | M | N |
| ATOM | 4979 | CA | VAL | M | 19 | 31.950 | 1.018 | 22.271 | 1.00 | 28.75 | M | C |
| ATOM | 4980 | CB | VAL | M | 19 | 32.513 | 2.408 | 21.903 | 1.00 | 28.85 | M | C |
| ATOM | 4981 | CG1 | VAL | M | 19 | 33.553 | 2.277 | 20.807 | 1.00 | 29.50 | M | C |
| ATOM | 4982 | CG2 | VAL | M | 19 | 31.431 | 3.342 | 21.509 | 1.00 | 33.02 | M | C |
| ATOM | 4983 | C | VAL | M | 19 | 31.121 | 1.110 | 23.521 | 1.00 | 29.88 | M | C |
| ATOM | 4984 | O | VAL | M | 19 | 29.898 | 1.295 | 23.490 | 1.00 | 31.32 | M | O |
| ATOM | 4985 | N | THR | M | 20 | 31.845 | 1.040 | 24.629 | 1.00 | 29.91 | M | N |
| ATOM | 4986 | CA | THR | M | 20 | 31.289 | 1.043 | 25.973 | 1.00 | 24.78 | M | C |
| ATOM | 4987 | CB | THR | M | 20 | 31.569 | −0.332 | 26.573 | 1.00 | 21.76 | M | C |
| ATOM | 4988 | OG1 | THR | M | 20 | 30.598 | −1.253 | 26.067 | 1.00 | 34.71 | M | O |
| ATOM | 4989 | CG2 | THR | M | 20 | 31.564 | −0.321 | 28.079 | 1.00 | 33.53 | M | C |
| ATOM | 4990 | C | THR | M | 20 | 31.845 | 2.153 | 26.877 | 1.00 | 21.73 | M | C |
| ATOM | 4991 | O | THR | M | 20 | 33.051 | 2.299 | 27.019 | 1.00 | 24.35 | M | O |
| ATOM | 4992 | N | ILE | M | 21 | 30.949 | 2.931 | 27.478 | 1.00 | 23.46 | M | N |
| ATOM | 4993 | CA | ILE | M | 21 | 31.315 | 4.021 | 28.383 | 1.00 | 21.23 | M | C |
| ATOM | 4994 | CB | ILE | M | 21 | 30.888 | 5.404 | 27.808 | 1.00 | 22.27 | M | C |
| ATOM | 4995 | CG2 | ILE | M | 21 | 31.194 | 6.519 | 28.800 | 1.00 | 17.64 | M | C |
| ATOM | 4996 | CG1 | ILE | M | 21 | 31.629 | 5.674 | 26.489 | 1.00 | 21.29 | M | C |
| ATOM | 4997 | CD1 | ILE | M | 21 | 31.326 | 7.012 | 25.871 | 1.00 | 17.66 | M | C |
| ATOM | 4998 | C | ILE | M | 21 | 30.589 | 3.793 | 29.701 | 1.00 | 24.42 | M | C |
| ATOM | 4999 | O | ILE | M | 21 | 29.358 | 3.689 | 29.727 | 1.00 | 20.07 | M | O |
| ATOM | 5000 | N | THR | M | 22 | 31.337 | 3.734 | 30.801 | 1.00 | 25.55 | M | N |
| ATOM | 5001 | CA | THR | M | 22 | 30.728 | 3.513 | 32.113 | 1.00 | 22.62 | M | C |
| ATOM | 5002 | CB | THR | M | 22 | 31.373 | 2.322 | 32.830 | 1.00 | 20.39 | M | C |
| ATOM | 5003 | OG1 | THR | M | 22 | 31.103 | 1.141 | 32.074 | 1.00 | 27.94 | M | O |
| ATOM | 5004 | CG2 | THR | M | 22 | 30.765 | 2.114 | 34.196 | 1.00 | 30.75 | M | C |
| ATOM | 5005 | C | THR | M | 22 | 30.689 | 4.730 | 33.022 | 1.00 | 21.70 | M | C |
| ATOM | 5006 | O | THR | M | 22 | 31.474 | 5.670 | 32.872 | 1.00 | 20.68 | M | O |
| ATOM | 5007 | N | CYS | M | 23 | 29.722 | 4.722 | 33.931 | 1.00 | 17.42 | M | N |
| ATOM | 5008 | CA | CYS | M | 23 | 29.533 | 5.807 | 34.882 | 1.00 | 28.54 | M | C |
| ATOM | 5009 | C | CYS | M | 23 | 29.250 | 5.125 | 36.234 | 1.00 | 27.76 | M | C |
| ATOM | 5010 | O | CYS | M | 23 | 28.497 | 4.157 | 36.301 | 1.00 | 24.34 | M | O |
| ATOM | 5011 | CB | CYS | M | 23 | 28.365 | 6.704 | 34.415 | 1.00 | 25.43 | M | C |
| ATOM | 5012 | SG | CYS | M | 23 | 28.060 | 8.261 | 35.317 | 1.00 | 50.17 | M | S |
| ATOM | 5013 | N | ARG | M | 24 | 29.947 | 5.555 | 37.283 | 1.00 | 23.05 | M | N |
| ATOM | 5014 | CA | ARG | M | 24 | 29.751 | 4.992 | 38.614 | 1.00 | 19.98 | M | C |
| ATOM | 5015 | CB | ARG | M | 24 | 30.965 | 4.167 | 39.072 | 1.00 | 21.26 | M | C |
| ATOM | 5016 | CG | ARG | M | 24 | 30.854 | 3.681 | 40.523 | 1.00 | 28.16 | M | C |
| ATOM | 5017 | CD | ARG | M | 24 | 31.935 | 2.661 | 40.934 | 1.00 | 29.34 | M | C |
| ATOM | 5018 | NE | ARG | M | 24 | 31.543 | 1.294 | 40.591 | 1.00 | 34.67 | M | N |
| ATOM | 5019 | CZ | ARG | M | 24 | 32.304 | 0.211 | 40.733 | 1.00 | 34.49 | M | C |
| ATOM | 5020 | NH1 | ARG | M | 24 | 33.537 | 0.304 | 41.225 | 1.00 | 34.31 | M | N |
| ATOM | 5021 | NH2 | ARG | M | 24 | 31.842 | −0.967 | 40.326 | 1.00 | 31.14 | M | N |
| ATOM | 5022 | C | ARG | M | 24 | 29.427 | 6.076 | 39.637 | 1.00 | 20.59 | M | C |
| ATOM | 5023 | O | ARG | M | 24 | 30.155 | 7.059 | 39.783 | 1.00 | 21.15 | M | O |
| ATOM | 5024 | N | ALA | M | 25 | 28.278 | 5.931 | 40.283 | 1.00 | 21.64 | M | N |
| ATOM | 5025 | CA | ALA | M | 25 | 27.865 | 6.886 | 41.297 | 1.00 | 20.98 | M | C |
| ATOM | 5026 | CB | ALA | M | 25 | 26.377 | 6.978 | 41.382 | 1.00 | 19.79 | M | C |
| ATOM | 5027 | C | ALA | M | 25 | 28.419 | 6.404 | 42.610 | 1.00 | 22.36 | M | C |
| ATOM | 5028 | O | ALA | M | 25 | 28.716 | 5.215 | 42.765 | 1.00 | 19.43 | M | O |
| ATOM | 5029 | N | SER | M | 26 | 28.612 | 7.340 | 43.533 | 1.00 | 22.98 | M | N |
| ATOM | 5030 | CA | SER | M | 26 | 29.135 | 7.009 | 44.852 | 1.00 | 26.80 | M | C |
| ATOM | 5031 | CB | SER | M | 26 | 29.598 | 8.265 | 45.609 | 1.00 | 23.90 | M | C |
| ATOM | 5032 | OG | SER | M | 26 | 28.710 | 9.362 | 45.461 | 1.00 | 28.66 | M | O |
| ATOM | 5033 | C | SER | M | 26 | 28.118 | 6.204 | 45.662 | 1.00 | 33.14 | M | C |
| ATOM | 5034 | O | SER | M | 26 | 28.493 | 5.479 | 46.581 | 1.00 | 41.52 | M | O |
| ATOM | 5035 | N | ARG | M | 27 | 26.838 | 6.311 | 45.303 | 1.00 | 33.35 | M | N |
| ATOM | 5036 | CA | ARG | M | 27 | 25.761 | 5.556 | 45.963 | 1.00 | 32.70 | M | C |
| ATOM | 5037 | CB | ARG | M | 27 | 25.217 | 6.280 | 47.201 | 1.00 | 23.97 | M | C |
| ATOM | 5038 | CG | ARG | M | 27 | 25.009 | 7.757 | 46.986 | 1.00 | 27.23 | M | C |
| ATOM | 5039 | CD | ARG | M | 27 | 23.963 | 8.320 | 47.889 | 1.00 | 22.43 | M | C |
| ATOM | 5040 | NE | ARG | M | 27 | 22.652 | 8.131 | 47.310 | 1.00 | 27.02 | M | N |
| ATOM | 5041 | CZ | ARG | M | 27 | 21.704 | 9.061 | 47.296 | 1.00 | 31.79 | M | C |
| ATOM | 5042 | NH1 | ARG | M | 27 | 21.918 | 10.241 | 47.861 | 1.00 | 24.32 | M | N |
| ATOM | 5043 | NH2 | ARG | M | 27 | 20.592 | 8.854 | 46.597 | 1.00 | 30.34 | M | N |
| ATOM | 5044 | C | ARG | M | 27 | 24.615 | 5.325 | 44.993 | 1.00 | 29.91 | M | C |
| ATOM | 5045 | O | ARG | M | 27 | 24.584 | 5.903 | 43.916 | 1.00 | 37.81 | M | O |
| ATOM | 5046 | N | ASP | M | 28 | 23.665 | 4.494 | 45.404 | 1.00 | 28.39 | M | N |
| ATOM | 5047 | CA | ASP | M | 28 | 22.476 | 4.151 | 44.620 | 1.00 | 24.76 | M | C |
| ATOM | 5048 | CB | ASP | M | 28 | 21.554 | 3.272 | 45.481 | 1.00 | 23.40 | M | C |
| ATOM | 5049 | CG | ASP | M | 28 | 20.479 | 2.532 | 44.665 | 1.00 | 32.72 | M | C |
| ATOM | 5050 | OD1 | ASP | M | 28 | 20.171 | 2.914 | 43.514 | 1.00 | 35.63 | M | O |
| ATOM | 5051 | OD2 | ASP | M | 28 | 19.914 | 1.561 | 45.202 | 1.00 | 34.41 | M | O |
| ATOM | 5052 | C | ASP | M | 28 | 21.711 | 5.394 | 44.136 | 1.00 | 23.37 | M | C |
| ATOM | 5053 | O | ASP | M | 28 | 21.302 | 6.229 | 44.944 | 1.00 | 21.68 | M | O |
| ATOM | 5054 | N | ILE | M | 29 | 21.527 | 5.521 | 42.822 | 1.00 | 15.24 | M | N |
| ATOM | 5055 | CA | ILE | M | 29 | 20.816 | 6.679 | 42.296 | 1.00 | 24.64 | M | C |
| ATOM | 5056 | CB | ILE | M | 29 | 21.697 | 7.512 | 41.339 | 1.00 | 21.99 | M | C |
| ATOM | 5057 | CG2 | ILE | M | 29 | 22.821 | 8.220 | 42.108 | 1.00 | 24.04 | M | C |
| ATOM | 5058 | CG1 | ILE | M | 29 | 22.312 | 6.613 | 40.274 | 1.00 | 25.49 | M | C |
| ATOM | 5059 | CD1 | ILE | M | 29 | 22.765 | 7.377 | 39.046 | 1.00 | 26.09 | M | C |
| ATOM | 5060 | C | ILE | M | 29 | 19.453 | 6.371 | 41.654 | 1.00 | 24.68 | M | C |
| ATOM | 5061 | O | ILE | M | 29 | 18.908 | 7.203 | 40.930 | 1.00 | 22.22 | M | O |
| ATOM | 5062 | N | LYS | M | 30 | 18.900 | 5.195 | 41.971 | 1.00 | 28.98 | M | N |
| ATOM | 5063 | CA | LYS | M | 30 | 17.580 | 4.739 | 41.484 | 1.00 | 30.47 | M | C |
| ATOM | 5064 | CB | LYS | M | 30 | 16.449 | 5.492 | 42.206 | 1.00 | 28.24 | M | C |
| ATOM | 5065 | CG | LYS | M | 30 | 16.086 | 5.019 | 43.602 | 1.00 | 36.53 | M | C |
| ATOM | 5066 | CD | LYS | M | 30 | 14.624 | 5.375 | 43.936 | 1.00 | 38.03 | M | C |
| ATOM | 5067 | CE | LYS | M | 30 | 13.667 | 4.669 | 42.948 | 1.00 | 35.64 | M | C |
| ATOM | 5068 | NZ | LYS | M | 30 | 12.260 | 5.132 | 42.972 | 1.00 | 34.01 | M | N |
| ATOM | 5069 | C | LYS | M | 30 | 17.343 | 4.865 | 39.972 | 1.00 | 26.57 | M | C |
| ATOM | 5070 | O | LYS | M | 30 | 16.242 | 5.192 | 39.538 | 1.00 | 24.41 | M | O |
| ATOM | 5071 | N | SER | M | 31 | 18.366 | 4.610 | 39.174 | 1.00 | 25.23 | M | N |
| ATOM | 5072 | CA | SER | M | 31 | 18.245 | 4.736 | 37.727 | 1.00 | 25.18 | M | C |
| ATOM | 5073 | CB | SER | M | 31 | 17.192 | 3.773 | 37.174 | 1.00 | 25.09 | M | C |
| ATOM | 5074 | OG | SER | M | 31 | 17.673 | 2.442 | 37.190 | 1.00 | 40.38 | M | O |
| ATOM | 5075 | C | SER | M | 31 | 17.966 | 6.165 | 37.240 | 1.00 | 19.83 | M | C |
| ATOM | 5076 | O | SER | M | 31 | 17.661 | 6.358 | 36.082 | 1.00 | 27.07 | M | O |
| ATOM | 5077 | N | TYR | M | 32 | 18.065 | 7.161 | 38.111 | 1.00 | 17.30 | M | N |
| ATOM | 5078 | CA | TYR | M | 32 | 17.857 | 8.550 | 37.703 | 1.00 | 20.57 | M | C |
| ATOM | 5079 | CB | TYR | M | 32 | 17.552 | 9.414 | 38.916 | 1.00 | 22.62 | M | C |
| ATOM | 5080 | CG | TYR | M | 32 | 16.329 | 8.983 | 39.681 | 1.00 | 33.07 | M | C |
| ATOM | 5081 | CD1 | TYR | M | 32 | 15.343 | 8.186 | 39.078 | 1.00 | 31.78 | M | C |
| ATOM | 5082 | CE1 | TYR | M | 32 | 14.208 | 7.783 | 39.780 | 1.00 | 27.54 | M | C |
| ATOM | 5083 | CD2 | TYR | M | 32 | 16.148 | 9.366 | 41.006 | 1.00 | 30.38 | M | C |
| ATOM | 5084 | CE2 | TYR | M | 32 | 15.013 | 8.966 | 41.713 | 1.00 | 33.76 | M | C |
| ATOM | 5085 | CZ | TYR | M | 32 | 14.052 | 8.176 | 41.095 | 1.00 | 30.91 | M | C |
| ATOM | 5086 | OH | TYR | M | 32 | 12.948 | 7.768 | 41.806 | 1.00 | 33.07 | M | O |
| ATOM | 5087 | C | TYR | M | 32 | 19.178 | 9.005 | 37.083 | 1.00 | 25.08 | M | C |
| ATOM | 5088 | O | TYR | M | 32 | 19.906 | 9.800 | 37.670 | 1.00 | 21.72 | M | O |
| ATOM | 5089 | N | LEU | M | 33 | 19.441 | 8.527 | 35.869 | 1.00 | 26.30 | M | N |
| ATOM | 5090 | CA | LEU | M | 33 | 20.676 | 8.788 | 35.148 | 1.00 | 18.66 | M | C |
| ATOM | 5091 | CB | LEU | M | 33 | 21.548 | 7.538 | 35.298 | 1.00 | 21.00 | M | C |
| ATOM | 5092 | CG | LEU | M | 33 | 23.011 | 7.443 | 34.861 | 1.00 | 23.21 | M | C |
| ATOM | 5093 | CD1 | LEU | M | 33 | 23.141 | 7.386 | 33.357 | 1.00 | 21.95 | M | C |
| ATOM | 5094 | CD2 | LEU | M | 33 | 23.776 | 8.592 | 35.439 | 1.00 | 18.20 | M | C |
| ATOM | 5095 | C | LEU | M | 33 | 20.364 | 9.025 | 33.663 | 1.00 | 23.53 | M | C |
| ATOM | 5096 | O | LEU | M | 33 | 19.664 | 8.227 | 33.039 | 1.00 | 17.81 | M | O |
| ATOM | 5097 | N | ASN | M | 34 | 20.928 | 10.096 | 33.102 | 1.00 | 21.85 | M | N |
| ATOM | 5098 | CA | ASN | M | 34 | 20.742 | 10.460 | 31.693 | 1.00 | 20.24 | M | C |
| ATOM | 5099 | CB | ASN | M | 34 | 20.168 | 11.868 | 31.578 | 1.00 | 17.15 | M | C |
| ATOM | 5100 | CG | ASN | M | 34 | 18.861 | 12.019 | 32.322 | 1.00 | 20.99 | M | C |
| ATOM | 5101 | OD1 | ASN | M | 34 | 18.042 | 11.116 | 32.329 | 1.00 | 25.08 | M | O |
| ATOM | 5102 | ND2 | ASN | M | 34 | 18.670 | 13.154 | 32.963 | 1.00 | 17.02 | M | N |
| ATOM | 5103 | C | ASN | M | 34 | 22.090 | 10.438 | 30.991 | 1.00 | 20.11 | M | C |
| ATOM | 5104 | O | ASN | M | 34 | 23.119 | 10.425 | 31.657 | 1.00 | 18.67 | M | O |
| ATOM | 5105 | N | TRP | M | 35 | 22.089 | 10.373 | 29.659 | 1.00 | 9.98 | M | N |
| ATOM | 5106 | CA | TRP | M | 35 | 23.348 | 10.396 | 28.906 | 1.00 | 14.02 | M | C |
| ATOM | 5107 | CB | TRP | M | 35 | 23.666 | 9.047 | 28.249 | 1.00 | 14.31 | M | C |
| ATOM | 5108 | CG | TRP | M | 35 | 24.115 | 7.952 | 29.167 | 1.00 | 11.92 | M | C |
| ATOM | 5109 | CD2 | TRP | M | 35 | 25.444 | 7.713 | 29.640 | 1.00 | 13.43 | M | C |
| ATOM | 5110 | CE2 | TRP | M | 35 | 25.408 | 6.527 | 30.402 | 1.00 | 11.84 | M | C |
| ATOM | 5111 | CE3 | TRP | M | 35 | 26.668 | 8.381 | 29.485 | 1.00 | 14.05 | M | C |
| ATOM | 5112 | CD1 | TRP | M | 35 | 23.352 | 6.942 | 29.653 | 1.00 | 15.54 | M | C |
| ATOM | 5113 | NE1 | TRP | M | 35 | 24.118 | 6.077 | 30.394 | 1.00 | 20.82 | M | N |
| ATOM | 5114 | CZ2 | TRP | M | 35 | 26.543 | 5.990 | 31.013 | 1.00 | 14.90 | M | C |
| ATOM | 5115 | CZ3 | TRP | M | 35 | 27.810 | 7.845 | 30.096 | 1.00 | 9.33 | M | C |
| ATOM | 5116 | CH2 | TRP | M | 35 | 27.735 | 6.660 | 30.850 | 1.00 | 13.39 | M | C |
| ATOM | 5117 | C | TRP | M | 35 | 23.267 | 11.469 | 27.831 | 1.00 | 13.25 | M | C |
| ATOM | 5118 | O | TRP | M | 35 | 22.236 | 11.627 | 27.179 | 1.00 | 14.50 | M | O |
| ATOM | 5119 | N | TYR | M | 36 | 24.361 | 12.195 | 27.629 | 1.00 | 14.27 | M | N |
| ATOM | 5120 | CA | TYR | M | 36 | 24.390 | 13.263 | 26.626 | 1.00 | 13.31 | M | C |
| ATOM | 5121 | CB | TYR | M | 36 | 24.414 | 14.659 | 27.286 | 1.00 | 12.84 | M | C |
| ATOM | 5122 | CG | TYR | M | 36 | 23.146 | 14.986 | 28.027 | 1.00 | 14.36 | M | C |
| ATOM | 5123 | CD1 | TYR | M | 36 | 22.954 | 14.560 | 29.335 | 1.00 | 13.95 | M | C |
| ATOM | 5124 | CE1 | TYR | M | 36 | 21.729 | 14.749 | 29.980 | 1.00 | 19.02 | M | C |
| ATOM | 5125 | CD2 | TYR | M | 36 | 22.090 | 15.622 | 27.383 | 1.00 | 15.37 | M | C |
| ATOM | 5126 | CE2 | TYR | M | 36 | 20.859 | 15.814 | 28.015 | 1.00 | 13.99 | M | C |
| ATOM | 5127 | CZ | TYR | M | 36 | 20.686 | 15.372 | 29.312 | 1.00 | 12.14 | M | C |
| ATOM | 5128 | OH | TYR | M | 36 | 19.470 | 15.531 | 29.928 | 1.00 | 15.47 | M | O |
| ATOM | 5129 | C | TYR | M | 36 | 25.578 | 13.163 | 25.724 | 1.00 | 12.47 | M | C |
| ATOM | 5130 | O | TYR | M | 36 | 26.624 | 12.628 | 26.102 | 1.00 | 22.55 | M | O |
| ATOM | 5131 | N | GLN | M | 37 | 25.398 | 13.696 | 24.527 | 1.00 | 15.32 | M | N |
| ATOM | 5132 | CA | GLN | M | 37 | 26.429 | 13.761 | 23.519 | 1.00 | 9.03 | M | C |
| ATOM | 5133 | CB | GLN | M | 37 | 25.943 | 13.135 | 22.215 | 1.00 | 18.78 | M | C |
| ATOM | 5134 | CG | GLN | M | 37 | 26.884 | 13.362 | 21.023 | 1.00 | 12.73 | M | C |
| ATOM | 5135 | CD | GLN | M | 37 | 26.274 | 12.910 | 19.695 | 1.00 | 18.74 | M | C |
| ATOM | 5136 | OE1 | GLN | M | 37 | 25.403 | 13.578 | 19.152 | 1.00 | 17.69 | M | O |
| ATOM | 5137 | NE2 | GLN | M | 37 | 26.743 | 11.777 | 19.166 | 1.00 | 12.25 | M | N |
| ATOM | 5138 | C | GLN | M | 37 | 26.658 | 15.246 | 23.279 | 1.00 | 15.46 | M | C |
| ATOM | 5139 | O | GLN | M | 37 | 25.706 | 16.036 | 23.248 | 1.00 | 13.11 | M | O |
| ATOM | 5140 | N | GLN | M | 38 | 27.914 | 15.636 | 23.136 | 1.00 | 15.58 | M | N |
| ATOM | 5141 | CA | GLN | M | 38 | 28.228 | 17.030 | 22.870 | 1.00 | 15.93 | M | C |
| ATOM | 5142 | CB | GLN | M | 38 | 28.613 | 17.793 | 24.146 | 1.00 | 9.88 | M | C |
| ATOM | 5143 | CG | GLN | M | 38 | 28.943 | 19.252 | 23.848 | 1.00 | 7.32 | M | C |
| ATOM | 5144 | CD | GLN | M | 38 | 29.190 | 20.083 | 25.085 | 1.00 | 9.54 | M | C |
| ATOM | 5145 | OE1 | GLN | M | 38 | 29.877 | 19.659 | 26.004 | 1.00 | 15.18 | M | O |
| ATOM | 5146 | NE2 | GLN | M | 38 | 28.645 | 21.290 | 25.100 | 1.00 | 3.56 | M | N |
| ATOM | 5147 | C | GLN | M | 38 | 29.358 | 17.114 | 21.872 | 1.00 | 15.50 | M | C |
| ATOM | 5148 | O | GLN | M | 38 | 30.310 | 16.336 | 21.937 | 1.00 | 13.95 | M | O |
| ATOM | 5149 | N | LYS | M | 39 | 29.208 | 18.031 | 20.921 | 1.00 | 16.52 | M | N |
| ATOM | 5150 | CA | LYS | M | 39 | 30.218 | 18.259 | 19.905 | 1.00 | 26.94 | M | C |
| ATOM | 5151 | CB | LYS | M | 39 | 29.634 | 18.075 | 18.499 | 1.00 | 28.00 | M | C |
| ATOM | 5152 | CG | LYS | M | 39 | 29.267 | 16.622 | 18.186 | 1.00 | 28.44 | M | C |
| ATOM | 5153 | CD | LYS | M | 39 | 28.762 | 16.447 | 16.766 | 1.00 | 24.95 | M | C |
| ATOM | 5154 | CE | LYS | M | 39 | 28.288 | 15.028 | 16.531 | 1.00 | 23.35 | M | C |
| ATOM | 5155 | NZ | LYS | M | 39 | 27.620 | 14.890 | 15.186 | 1.00 | 26.95 | M | N |
| ATOM | 5156 | C | LYS | M | 39 | 30.750 | 19.669 | 20.121 | 1.00 | 28.60 | M | C |
| ATOM | 5157 | O | LYS | M | 39 | 30.056 | 20.517 | 20.673 | 1.00 | 29.99 | M | O |
| ATOM | 5158 | N | PRO | M | 40 | 31.994 | 19.931 | 19.697 | 1.00 | 27.56 | M | N |
| ATOM | 5159 | CD | PRO | M | 40 | 32.827 | 18.977 | 18.939 | 1.00 | 27.66 | M | C |
| ATOM | 5160 | CA | PRO | M | 40 | 32.673 | 21.222 | 19.828 | 1.00 | 24.58 | M | C |
| ATOM | 5161 | CB | PRO | M | 40 | 33.837 | 21.076 | 18.856 | 1.00 | 30.74 | M | C |
| ATOM | 5162 | CG | PRO | M | 40 | 34.188 | 19.604 | 18.997 | 1.00 | 21.40 | M | C |
| ATOM | 5163 | C | PRO | M | 40 | 31.843 | 22.465 | 19.515 | 1.00 | 27.06 | M | C |
| ATOM | 5164 | O | PRO | M | 40 | 31.343 | 22.618 | 18.405 | 1.00 | 24.61 | M | O |
| ATOM | 5165 | N | GLY | M | 41 | 31.710 | 23.354 | 20.505 | 1.00 | 27.78 | M | N |
| ATOM | 5166 | CA | GLY | M | 41 | 30.962 | 24.590 | 20.320 | 1.00 | 21.69 | M | C |
| ATOM | 5167 | C | GLY | M | 41 | 29.439 | 24.519 | 20.282 | 1.00 | 29.87 | M | C |
| ATOM | 5168 | O | GLY | M | 41 | 28.790 | 25.557 | 20.112 | 1.00 | 27.28 | M | O |
| ATOM | 5169 | N | LYS | M | 42 | 28.870 | 23.317 | 20.433 | 1.00 | 25.82 | M | N |
| ATOM | 5170 | CA | LYS | M | 42 | 27.412 | 23.116 | 20.409 | 1.00 | 23.84 | M | C |
| ATOM | 5171 | CB | LYS | M | 42 | 27.019 | 22.009 | 19.408 | 1.00 | 24.68 | M | C |
| ATOM | 5172 | CG | LYS | M | 42 | 27.568 | 22.160 | 17.989 | 1.00 | 27.29 | M | C |
| ATOM | 5173 | CD | LYS | M | 42 | 27.151 | 23.479 | 17.383 | 1.00 | 32.77 | M | C |
| ATOM | 5174 | CE | LYS | M | 42 | 27.637 | 23.606 | 15.951 | 1.00 | 33.71 | M | C |
| ATOM | 5175 | NZ | LYS | M | 42 | 27.527 | 25.032 | 15.477 | 1.00 | 43.72 | M | N |
| ATOM | 5176 | C | LYS | M | 42 | 26.855 | 22.720 | 21.776 | 1.00 | 20.45 | M | C |
| ATOM | 5177 | O | LYS | M | 42 | 27.585 | 22.301 | 22.671 | 1.00 | 22.73 | M | O |
| ATOM | 5178 | N | ALA | M | 43 | 25.546 | 22.860 | 21.927 | 1.00 | 20.53 | M | N |
| ATOM | 5179 | CA | ALA | M | 43 | 24.860 | 22.466 | 23.150 | 1.00 | 18.94 | M | C |
| ATOM | 5180 | CB | ALA | M | 43 | 23.447 | 23.036 | 23.149 | 1.00 | 7.85 | M | C |
| ATOM | 5181 | C | ALA | M | 43 | 24.798 | 20.922 | 23.185 | 1.00 | 17.52 | M | C |
| ATOM | 5182 | O | ALA | M | 43 | 24.862 | 20.263 | 22.161 | 1.00 | 21.19 | M | O |
| ATOM | 5183 | N | PRO | M | 44 | 24.678 | 20.333 | 24.374 | 1.00 | 22.01 | M | N |
| ATOM | 5184 | CD | PRO | M | 44 | 24.771 | 20.891 | 25.730 | 1.00 | 14.50 | M | C |
| ATOM | 5185 | CA | PRO | M | 44 | 24.609 | 18.875 | 24.432 | 1.00 | 20.18 | M | C |
| ATOM | 5186 | CB | PRO | M | 44 | 24.682 | 18.600 | 25.943 | 1.00 | 15.93 | M | C |
| ATOM | 5187 | CG | PRO | M | 44 | 25.403 | 19.751 | 26.463 | 1.00 | 15.27 | M | C |
| ATOM | 5188 | C | PRO | M | 44 | 23.276 | 18.376 | 23.859 | 1.00 | 20.04 | M | C |
| ATOM | 5189 | O | PRO | M | 44 | 22.323 | 19.139 | 23.695 | 1.00 | 17.73 | M | O |
| ATOM | 5190 | N | LYS | M | 45 | 23.187 | 17.072 | 23.655 | 1.00 | 20.15 | M | N |
| ATOM | 5191 | CA | LYS | M | 45 | 21.970 | 16.472 | 23.124 | 1.00 | 18.87 | M | C |
| ATOM | 5192 | CB | LYS | M | 45 | 22.213 | 16.029 | 21.688 | 1.00 | 25.24 | M | C |
| ATOM | 5193 | CG | LYS | M | 45 | 21.370 | 16.639 | 20.622 | 1.00 | 30.45 | M | C |
| ATOM | 5194 | CD | LYS | M | 45 | 21.683 | 15.900 | 19.297 | 1.00 | 40.28 | M | C |
| ATOM | 5195 | CE | LYS | M | 45 | 21.603 | 14.355 | 19.462 | 1.00 | 41.19 | M | C |
| ATOM | 5196 | NZ | LYS | M | 45 | 22.069 | 13.530 | 18.278 | 1.00 | 38.57 | M | N |
| ATOM | 5197 | C | LYS | M | 45 | 21.724 | 15.233 | 23.945 | 1.00 | 15.54 | M | C |
| ATOM | 5198 | O | LYS | M | 45 | 22.659 | 14.473 | 24.198 | 1.00 | 7.44 | M | O |
| ATOM | 5199 | N | VAL | M | 46 | 20.471 | 15.014 | 24.337 | 1.00 | 16.26 | M | N |
| ATOM | 5200 | CA | VAL | M | 46 | 20.093 | 13.829 | 25.108 | 1.00 | 11.20 | M | C |
| ATOM | 5201 | CB | VAL | M | 46 | 18.682 | 13.981 | 25.708 | 1.00 | 9.87 | M | C |
| ATOM | 5202 | CG1 | VAL | M | 46 | 17.654 | 13.960 | 24.636 | 1.00 | 4.07 | M | C |
| ATOM | 5203 | CG2 | VAL | M | 46 | 18.412 | 12.886 | 26.694 | 1.00 | 8.82 | M | C |
| ATOM | 5204 | C | VAL | M | 46 | 20.105 | 12.588 | 24.214 | 1.00 | 13.26 | M | C |
| ATOM | 5205 | O | VAL | M | 46 | 19.816 | 12.686 | 23.031 | 1.00 | 17.79 | M | O |
| ATOM | 5206 | N | LEU | M | 47 | 20.490 | 11.442 | 24.776 | 1.00 | 12.89 | M | N |
| ATOM | 5207 | CA | LEU | M | 47 | 20.519 | 10.171 | 24.055 | 1.00 | 8.44 | M | C |
| ATOM | 5208 | CB | LEU | M | 47 | 21.930 | 9.599 | 23.979 | 1.00 | 7.40 | M | C |
| ATOM | 5209 | CG | LEU | M | 47 | 23.052 | 10.485 | 23.437 | 1.00 | 16.40 | M | C |
| ATOM | 5210 | CD1 | LEU | M | 47 | 24.376 | 9.988 | 24.009 | 1.00 | 6.27 | M | C |
| ATOM | 5211 | CD2 | LEU | M | 47 | 23.068 | 10.487 | 21.897 | 1.00 | 9.57 | M | C |
| ATOM | 5212 | C | LEU | M | 47 | 19.684 | 9.179 | 24.853 | 1.00 | 10.15 | M | C |
| ATOM | 5213 | O | LEU | M | 47 | 18.797 | 8.531 | 24.324 | 1.00 | 17.57 | M | O |
| ATOM | 5214 | N | ILE | M | 48 | 19.987 | 9.068 | 26.141 | 1.00 | 11.29 | M | N |
| ATOM | 5215 | CA | ILE | M | 48 | 19.315 | 8.135 | 27.028 | 1.00 | 10.27 | M | C |
| ATOM | 5216 | CB | ILE | M | 48 | 20.276 | 7.013 | 27.443 | 1.00 | 7.70 | M | C |
| ATOM | 5217 | CG2 | ILE | M | 48 | 19.555 | 5.972 | 28.227 | 1.00 | 16.40 | M | C |
| ATOM | 5218 | CG1 | ILE | M | 48 | 20.925 | 6.354 | 26.236 | 1.00 | 13.18 | M | C |
| ATOM | 5219 | CD1 | ILE | M | 48 | 20.027 | 5.434 | 25.473 | 1.00 | 16.06 | M | C |
| ATOM | 5220 | C | ILE | M | 48 | 18.831 | 8.815 | 28.320 | 1.00 | 17.87 | M | C |
| ATOM | 5221 | O | ILE | M | 48 | 19.482 | 9.722 | 28.838 | 1.00 | 13.52 | M | O |
| ATOM | 5222 | N | TYR | M | 49 | 17.668 | 8.390 | 28.817 | 1.00 | 19.50 | M | N |
| ATOM | 5223 | CA | TYR | M | 49 | 17.136 | 8.916 | 30.072 | 1.00 | 12.72 | M | C |
| ATOM | 5224 | CB | TYR | M | 49 | 16.030 | 9.956 | 29.858 | 1.00 | 14.57 | M | C |
| ATOM | 5225 | CG | TYR | M | 49 | 14.745 | 9.415 | 29.267 | 1.00 | 23.43 | M | C |
| ATOM | 5226 | CD1 | TYR | M | 49 | 14.631 | 9.160 | 27.892 | 1.00 | 22.57 | M | C |
| ATOM | 5227 | CE1 | TYR | M | 49 | 13.437 | 8.700 | 27.338 | 1.00 | 16.50 | M | C |
| ATOM | 5228 | CD2 | TYR | M | 49 | 13.628 | 9.195 | 30.075 | 1.00 | 21.34 | M | C |
| ATOM | 5229 | CE2 | TYR | M | 49 | 12.431 | 8.746 | 29.536 | 1.00 | 18.97 | M | C |
| ATOM | 5230 | CZ | TYR | M | 49 | 12.339 | 8.495 | 28.160 | 1.00 | 23.21 | M | C |
| ATOM | 5231 | OH | TYR | M | 49 | 11.148 | 8.033 | 27.628 | 1.00 | 19.00 | M | O |
| ATOM | 5232 | C | TYR | M | 49 | 16.644 | 7.733 | 30.883 | 1.00 | 11.96 | M | C |
| ATOM | 5233 | O | TYR | M | 49 | 16.450 | 6.640 | 30.341 | 1.00 | 12.70 | M | O |
| ATOM | 5234 | N | TYR | M | 50 | 16.552 | 7.924 | 32.196 | 1.00 | 13.56 | M | N |
| ATOM | 5235 | CA | TYR | M | 50 | 16.101 | 6.884 | 33.115 | 1.00 | 11.73 | M | C |
| ATOM | 5236 | CB | TYR | M | 50 | 14.615 | 6.598 | 32.918 | 1.00 | 14.22 | M | C |
| ATOM | 5237 | CG | TYR | M | 50 | 14.019 | 5.835 | 34.062 | 1.00 | 19.02 | M | C |
| ATOM | 5238 | CD1 | TYR | M | 50 | 14.206 | 6.262 | 35.395 | 1.00 | 18.40 | M | C |
| ATOM | 5239 | CE1 | TYR | M | 50 | 13.681 | 5.537 | 36.465 | 1.00 | 18.03 | M | C |
| ATOM | 5240 | CD2 | TYR | M | 50 | 13.296 | 4.673 | 33.838 | 1.00 | 12.39 | M | C |
| ATOM | 5241 | CE2 | TYR | M | 50 | 12.771 | 3.939 | 34.902 | 1.00 | 18.92 | M | C |
| ATOM | 5242 | CZ | TYR | M | 50 | 12.963 | 4.367 | 36.212 | 1.00 | 15.58 | M | C |
| ATOM | 5243 | OH | TYR | M | 50 | 12.453 | 3.612 | 37.257 | 1.00 | 17.30 | M | O |
| ATOM | 5244 | C | TYR | M | 50 | 16.917 | 5.607 | 32.988 | 1.00 | 15.53 | M | C |
| ATOM | 5245 | O | TYR | M | 50 | 16.387 | 4.498 | 33.072 | 1.00 | 19.76 | M | O |
| ATOM | 5246 | N | ALA | M | 51 | 18.221 | 5.790 | 32.781 | 1.00 | 15.74 | M | N |
| ATOM | 5247 | CA | ALA | M | 51 | 19.183 | 4.703 | 32.632 | 1.00 | 13.72 | M | C |
| ATOM | 5248 | CB | ALA | M | 51 | 19.151 | 3.784 | 33.844 | 1.00 | 8.85 | M | C |
| ATOM | 5249 | C | ALA | M | 51 | 19.093 | 3.860 | 31.380 | 1.00 | 18.82 | M | C |
| ATOM | 5250 | O | ALA | M | 51 | 20.125 | 3.479 | 30.822 | 1.00 | 14.80 | M | O |
| ATOM | 5251 | N | THR | M | 52 | 17.877 | 3.534 | 30.943 | 1.00 | 19.04 | M | N |
| ATOM | 5252 | CA | THR | M | 52 | 17.737 | 2.662 | 29.782 | 1.00 | 19.83 | M | C |
| ATOM | 5253 | CB | THR | M | 52 | 17.334 | 1.249 | 30.204 | 1.00 | 25.55 | M | C |
| ATOM | 5254 | OG1 | THR | M | 52 | 15.980 | 1.272 | 30.684 | 1.00 | 31.46 | M | O |
| ATOM | 5255 | CG2 | THR | M | 52 | 18.273 | 0.714 | 31.301 | 1.00 | 19.86 | M | C |
| ATOM | 5256 | C | THR | M | 52 | 16.783 | 3.071 | 28.672 | 1.00 | 21.68 | M | C |
| ATOM | 5257 | O | THR | M | 52 | 16.648 | 2.342 | 27.696 | 1.00 | 25.97 | M | O |
| ATOM | 5258 | N | SER | M | 53 | 16.139 | 4.225 | 28.786 | 1.00 | 21.55 | M | N |
| ATOM | 5259 | CA | SER | M | 53 | 15.218 | 4.660 | 27.740 | 1.00 | 19.19 | M | C |
| ATOM | 5260 | CB | SER | M | 53 | 14.073 | 5.445 | 28.350 | 1.00 | 23.02 | M | C |
| ATOM | 5261 | OG | SER | M | 53 | 13.538 | 4.709 | 29.437 | 1.00 | 31.58 | M | O |
| ATOM | 5262 | C | SER | M | 53 | 15.860 | 5.486 | 26.641 | 1.00 | 21.74 | M | C |
| ATOM | 5263 | O | SER | M | 53 | 16.502 | 6.515 | 26.892 | 1.00 | 22.54 | M | O |
| ATOM | 5264 | N | LEU | M | 54 | 15.675 | 5.016 | 25.413 | 1.00 | 25.45 | M | N |
| ATOM | 5265 | CA | LEU | M | 54 | 16.179 | 5.678 | 24.224 | 1.00 | 18.16 | M | C |
| ATOM | 5266 | CB | LEU | M | 54 | 16.046 | 4.743 | 23.038 | 1.00 | 16.92 | M | C |
| ATOM | 5267 | CG | LEU | M | 54 | 16.487 | 5.374 | 21.726 | 1.00 | 22.74 | M | C |
| ATOM | 5268 | CD1 | LEU | M | 54 | 17.972 | 5.692 | 21.840 | 1.00 | 16.30 | M | C |
| ATOM | 5269 | CD2 | LEU | M | 54 | 16.203 | 4.451 | 20.566 | 1.00 | 13.65 | M | C |
| ATOM | 5270 | C | LEU | M | 54 | 15.278 | 6.879 | 24.001 | 1.00 | 24.15 | M | C |
| ATOM | 5271 | O | LEU | M | 54 | 14.065 | 6.705 | 23.840 | 1.00 | 24.78 | M | O |
| ATOM | 5272 | N | ALA | M | 55 | 15.852 | 8.082 | 23.972 | 1.00 | 16.34 | M | N |
| ATOM | 5273 | CA | ALA | M | 55 | 15.049 | 9.292 | 23.786 | 1.00 | 20.54 | M | C |
| ATOM | 5274 | CB | ALA | M | 55 | 15.838 | 10.539 | 24.243 | 1.00 | 17.60 | M | C |
| ATOM | 5275 | C | ALA | M | 55 | 14.532 | 9.468 | 22.354 | 1.00 | 21.93 | M | C |
| ATOM | 5276 | O | ALA | M | 55 | 15.167 | 9.044 | 21.396 | 1.00 | 19.76 | M | O |
| ATOM | 5277 | N | GLU | M | 56 | 13.404 | 10.153 | 22.207 | 1.00 | 23.83 | M | N |
| ATOM | 5278 | CA | GLU | M | 56 | 12.822 | 10.350 | 20.889 | 1.00 | 32.35 | M | C |
| ATOM | 5279 | CB | GLU | M | 56 | 11.599 | 11.273 | 20.974 | 1.00 | 35.21 | M | C |
| ATOM | 5280 | CG | GLU | M | 56 | 10.833 | 11.423 | 19.656 | 1.00 | 40.75 | M | C |
| ATOM | 5281 | CD | GLU | M | 56 | 9.440 | 12.008 | 19.847 | 1.00 | 50.98 | M | C |
| ATOM | 5282 | OE1 | GLU | M | 56 | 9.333 | 13.251 | 19.979 | 1.00 | 53.43 | M | O |
| ATOM | 5283 | OE2 | GLU | M | 56 | 8.452 | 11.228 | 19.863 | 1.00 | 45.14 | M | O |
| ATOM | 5284 | C | GLU | M | 56 | 13.795 | 10.872 | 19.827 | 1.00 | 29.59 | M | C |
| ATOM | 5285 | O | GLU | M | 56 | 14.545 | 11.814 | 20.067 | 1.00 | 31.28 | M | O |
| ATOM | 5286 | N | GLY | M | 57 | 13.788 | 10.229 | 18.660 | 1.00 | 26.29 | M | N |
| ATOM | 5287 | CA | GLY | M | 57 | 14.637 | 10.667 | 17.562 | 1.00 | 21.22 | M | C |
| ATOM | 5288 | C | GLY | M | 57 | 16.095 | 10.261 | 17.610 | 1.00 | 21.45 | M | C |
| ATOM | 5289 | O | GLY | M | 57 | 16.836 | 10.520 | 16.675 | 1.00 | 33.65 | M | O |
| ATOM | 5290 | N | VAL | M | 58 | 16.506 | 9.599 | 18.681 | 1.00 | 21.40 | M | N |
| ATOM | 5291 | CA | VAL | M | 58 | 17.888 | 9.174 | 18.833 | 1.00 | 15.24 | M | C |
| ATOM | 5292 | CB | VAL | M | 58 | 18.266 | 9.056 | 20.379 | 1.00 | 19.58 | M | C |
| ATOM | 5293 | CG1 | VAL | M | 58 | 19.670 | 8.466 | 20.586 | 1.00 | 9.04 | M | C |
| ATOM | 5294 | CG2 | VAL | M | 58 | 18.170 | 10.429 | 21.066 | 1.00 | 9.55 | M | C |
| ATOM | 5295 | C | VAL | M | 58 | 18.142 | 7.843 | 18.107 | 1.00 | 17.79 | M | C |
| ATOM | 5296 | O | VAL | M | 58 | 17.360 | 6.901 | 18.206 | 1.00 | 24.08 | M | O |
| ATOM | 5297 | N | PRO | M | 59 | 19.224 | 7.768 | 17.331 | 1.00 | 15.38 | M | N |
| ATOM | 5298 | CD | PRO | M | 59 | 20.167 | 8.849 | 16.990 | 1.00 | 15.20 | M | C |
| ATOM | 5299 | CA | PRO | M | 59 | 19.554 | 6.541 | 16.610 | 1.00 | 14.81 | M | C |
| ATOM | 5300 | CB | PRO | M | 59 | 20.949 | 6.840 | 16.080 | 1.00 | 19.22 | M | C |
| ATOM | 5301 | CG | PRO | M | 59 | 20.859 | 8.287 | 15.739 | 1.00 | 12.53 | M | C |
| ATOM | 5302 | C | PRO | M | 59 | 19.576 | 5.363 | 17.573 | 1.00 | 25.46 | M | C |
| ATOM | 5303 | O | PRO | M | 59 | 20.097 | 5.469 | 18.701 | 1.00 | 26.42 | M | O |
| ATOM | 5304 | N | SER | M | 60 | 19.044 | 4.231 | 17.108 | 1.00 | 21.16 | M | N |
| ATOM | 5305 | CA | SER | M | 60 | 18.952 | 3.032 | 17.928 | 1.00 | 20.71 | M | C |
| ATOM | 5306 | CB | SER | M | 60 | 17.976 | 2.028 | 17.303 | 1.00 | 21.45 | M | C |
| ATOM | 5307 | OG | SER | M | 60 | 18.522 | 1.415 | 16.161 | 1.00 | 23.87 | M | O |
| ATOM | 5308 | C | SER | M | 60 | 20.257 | 2.342 | 18.305 | 1.00 | 18.30 | M | C |
| ATOM | 5309 | O | SER | M | 60 | 20.251 | 1.429 | 19.132 | 1.00 | 17.27 | M | O |
| ATOM | 5310 | N | ARG | M | 61 | 21.373 | 2.751 | 17.707 | 1.00 | 22.09 | M | N |
| ATOM | 5311 | CA | ARG | M | 61 | 22.663 | 2.139 | 18.052 | 1.00 | 18.50 | M | C |
| ATOM | 5312 | CB | ARG | M | 61 | 23.737 | 2.476 | 17.025 | 1.00 | 16.18 | M | C |
| ATOM | 5313 | CG | ARG | M | 61 | 23.989 | 3.976 | 16.840 | 1.00 | 19.71 | M | C |
| ATOM | 5314 | CD | ARG | M | 61 | 25.054 | 4.195 | 15.784 | 1.00 | 9.75 | M | C |
| ATOM | 5315 | NE | ARG | M | 61 | 25.231 | 5.600 | 15.471 | 1.00 | 11.95 | M | N |
| ATOM | 5316 | CZ | ARG | M | 61 | 24.482 | 6.301 | 14.633 | 1.00 | 12.80 | M | C |
| ATOM | 5317 | NH1 | ARG | M | 61 | 23.459 | 5.739 | 13.993 | 1.00 | 18.43 | M | N |
| ATOM | 5318 | NH2 | ARG | M | 61 | 24.761 | 7.575 | 14.433 | 1.00 | 7.60 | M | N |
| ATOM | 5319 | C | ARG | M | 61 | 23.111 | 2.568 | 19.456 | 1.00 | 19.14 | M | C |
| ATOM | 5320 | O | ARG | M | 61 | 24.025 | 1.976 | 20.033 | 1.00 | 11.44 | M | O |
| ATOM | 5321 | N | PHE | M | 62 | 22.477 | 3.618 | 19.978 | 1.00 | 13.64 | M | N |
| ATOM | 5322 | CA | PHE | M | 62 | 22.756 | 4.101 | 21.319 | 1.00 | 12.28 | M | C |
| ATOM | 5323 | CB | PHE | M | 62 | 22.463 | 5.596 | 21.409 | 1.00 | 8.83 | M | C |
| ATOM | 5324 | CG | PHE | M | 62 | 23.443 | 6.453 | 20.656 | 1.00 | 13.49 | M | C |
| ATOM | 5325 | CD1 | PHE | M | 62 | 24.661 | 6.815 | 21.232 | 1.00 | 13.94 | M | C |
| ATOM | 5326 | CD2 | PHE | M | 62 | 23.151 | 6.906 | 19.374 | 1.00 | 11.85 | M | C |
| ATOM | 5327 | CE1 | PHE | M | 62 | 25.576 | 7.620 | 20.539 | 1.00 | 11.76 | M | C |
| ATOM | 5328 | CE2 | PHE | M | 62 | 24.052 | 7.706 | 18.678 | 1.00 | 13.55 | M | C |
| ATOM | 5329 | CZ | PHE | M | 62 | 25.270 | 8.065 | 19.266 | 1.00 | 12.06 | M | C |
| ATOM | 5330 | C | PHE | M | 62 | 21.815 | 3.368 | 22.262 | 1.00 | 13.44 | M | C |
| ATOM | 5331 | O | PHE | M | 62 | 20.622 | 3.271 | 21.996 | 1.00 | 16.48 | M | O |
| ATOM | 5332 | N | SER | M | 63 | 22.357 | 2.793 | 23.330 | 1.00 | 17.69 | M | N |
| ATOM | 5333 | CA | SER | M | 63 | 21.533 | 2.099 | 24.313 | 1.00 | 19.15 | M | C |
| ATOM | 5334 | CB | SER | M | 63 | 21.360 | 0.607 | 23.968 | 1.00 | 15.50 | M | C |
| ATOM | 5335 | OG | SER | M | 63 | 22.493 | −0.167 | 24.311 | 1.00 | 28.40 | M | O |
| ATOM | 5336 | C | SER | M | 63 | 22.131 | 2.263 | 25.705 | 1.00 | 23.36 | M | C |
| ATOM | 5337 | O | SER | M | 63 | 23.362 | 2.358 | 25.860 | 1.00 | 19.47 | M | O |
| ATOM | 5338 | N | GLY | M | 64 | 21.246 | 2.320 | 26.700 | 1.00 | 16.20 | M | N |
| ATOM | 5339 | CA | GLY | M | 64 | 21.665 | 2.476 | 28.073 | 1.00 | 23.46 | M | C |
| ATOM | 5340 | C | GLY | M | 64 | 21.328 | 1.254 | 28.891 | 1.00 | 24.19 | M | C |
| ATOM | 5341 | O | GLY | M | 64 | 20.353 | 0.558 | 28.618 | 1.00 | 27.81 | M | O |
| ATOM | 5342 | N | SER | M | 65 | 22.145 | 0.993 | 29.901 | 1.00 | 23.29 | M | N |
| ATOM | 5343 | CA | SER | M | 65 | 21.932 | −0.149 | 30.767 | 1.00 | 25.61 | M | C |
| ATOM | 5344 | CB | SER | M | 65 | 22.662 | −1.359 | 30.213 | 1.00 | 23.36 | M | C |
| ATOM | 5345 | OG | SER | M | 65 | 22.411 | −2.482 | 31.027 | 1.00 | 47.46 | M | O |
| ATOM | 5346 | C | SER | M | 65 | 22.459 | 0.160 | 32.157 | 1.00 | 23.82 | M | C |
| ATOM | 5347 | O | SER | M | 65 | 23.241 | 1.094 | 32.330 | 1.00 | 25.57 | M | O |
| ATOM | 5348 | N | GLY | M | 66 | 22.017 | −0.618 | 33.139 | 1.00 | 22.41 | M | N |
| ATOM | 5349 | CA | GLY | M | 66 | 22.478 | −0.427 | 34.500 | 1.00 | 22.13 | M | C |
| ATOM | 5350 | C | GLY | M | 66 | 21.425 | −0.127 | 35.546 | 1.00 | 23.44 | M | C |
| ATOM | 5351 | O | GLY | M | 66 | 20.261 | 0.141 | 35.240 | 1.00 | 29.64 | M | O |
| ATOM | 5352 | N | SER | M | 67 | 21.850 | −0.173 | 36.801 | 1.00 | 25.18 | M | N |
| ATOM | 5353 | CA | SER | M | 67 | 20.978 | 0.101 | 37.933 | 1.00 | 28.54 | M | C |
| ATOM | 5354 | CB | SER | M | 67 | 19.998 | −1.061 | 38.152 | 1.00 | 29.68 | M | C |
| ATOM | 5355 | OG | SER | M | 67 | 20.702 | −2.282 | 38.372 | 1.00 | 28.60 | M | O |
| ATOM | 5356 | C | SER | M | 67 | 21.906 | 0.213 | 39.119 | 1.00 | 24.53 | M | C |
| ATOM | 5357 | O | SER | M | 67 | 23.060 | −0.213 | 39.037 | 1.00 | 31.79 | M | O |
| ATOM | 5358 | N | GLY | M | 68 | 21.392 | 0.740 | 40.228 | 1.00 | 27.54 | M | N |
| ATOM | 5359 | CA | GLY | M | 68 | 22.198 | 0.894 | 41.429 | 1.00 | 16.52 | M | C |
| ATOM | 5360 | C | GLY | M | 68 | 23.166 | 2.051 | 41.320 | 1.00 | 21.07 | M | C |
| ATOM | 5361 | O | GLY | M | 68 | 22.764 | 3.204 | 41.296 | 1.00 | 26.17 | M | O |
| ATOM | 5362 | N | THR | M | 69 | 24.446 | 1.733 | 41.209 | 1.00 | 21.88 | M | N |
| ATOM | 5363 | CA | THR | M | 69 | 25.495 | 2.741 | 41.104 | 1.00 | 22.25 | M | C |
| ATOM | 5364 | CB | THR | M | 69 | 26.562 | 2.550 | 42.217 | 1.00 | 23.98 | M | C |
| ATOM | 5365 | OG1 | THR | M | 69 | 27.174 | 1.260 | 42.062 | 1.00 | 18.07 | M | O |
| ATOM | 5366 | CG2 | THR | M | 69 | 25.949 | 2.639 | 43.604 | 1.00 | 21.25 | M | C |
| ATOM | 5367 | C | THR | M | 69 | 26.240 | 2.620 | 39.774 | 1.00 | 27.85 | M | C |
| ATOM | 5368 | O | THR | M | 69 | 27.000 | 3.523 | 39.412 | 1.00 | 27.80 | M | O |
| ATOM | 5369 | N | ASP | M | 70 | 26.048 | 1.497 | 39.074 | 1.00 | 24.36 | M | N |
| ATOM | 5370 | CA | ASP | M | 70 | 26.728 | 1.247 | 37.816 | 1.00 | 22.55 | M | C |
| ATOM | 5371 | CB | ASP | M | 70 | 27.443 | −0.101 | 37.859 | 1.00 | 28.59 | M | C |
| ATOM | 5372 | CG | ASP | M | 70 | 28.586 | −0.122 | 38.872 | 1.00 | 31.23 | M | C |
| ATOM | 5373 | OD1 | ASP | M | 70 | 28.908 | 0.947 | 39.424 | 1.00 | 29.55 | M | O |
| ATOM | 5374 | OD2 | ASP | M | 70 | 29.163 | −1.205 | 39.111 | 1.00 | 37.09 | M | O |
| ATOM | 5375 | C | ASP | M | 70 | 25.835 | 1.360 | 36.600 | 1.00 | 25.07 | M | C |
| ATOM | 5376 | O | ASP | M | 70 | 24.777 | 0.725 | 36.506 | 1.00 | 28.75 | M | O |
| ATOM | 5377 | N | TYR | M | 71 | 26.274 | 2.189 | 35.663 | 1.00 | 21.42 | M | N |
| ATOM | 5378 | CA | TYR | M | 71 | 25.525 | 2.443 | 34.451 | 1.00 | 19.08 | M | C |
| ATOM | 5379 | CB | TYR | M | 71 | 24.805 | 3.795 | 34.552 | 1.00 | 17.88 | M | C |
| ATOM | 5380 | CG | TYR | M | 71 | 23.813 | 3.827 | 35.695 | 1.00 | 20.51 | M | C |
| ATOM | 5381 | CD1 | TYR | M | 71 | 24.227 | 4.135 | 36.991 | 1.00 | 18.51 | M | C |
| ATOM | 5382 | CE1 | TYR | M | 71 | 23.352 | 4.089 | 38.059 | 1.00 | 20.70 | M | C |
| ATOM | 5383 | CD2 | TYR | M | 71 | 22.483 | 3.475 | 35.497 | 1.00 | 16.10 | M | C |
| ATOM | 5384 | CE2 | TYR | M | 71 | 21.599 | 3.425 | 36.561 | 1.00 | 23.96 | M | C |
| ATOM | 5385 | CZ | TYR | M | 71 | 22.038 | 3.732 | 37.844 | 1.00 | 22.73 | M | C |
| ATOM | 5386 | OH | TYR | M | 71 | 21.165 | 3.661 | 38.913 | 1.00 | 25.43 | M | O |
| ATOM | 5387 | C | TYR | M | 71 | 26.445 | 2.414 | 33.263 | 1.00 | 20.70 | M | C |
| ATOM | 5388 | O | TYR | M | 71 | 27.629 | 2.751 | 33.366 | 1.00 | 23.89 | M | O |
| ATOM | 5389 | N | THR | M | 72 | 25.895 | 2.019 | 32.123 | 1.00 | 18.19 | M | N |
| ATOM | 5390 | CA | THR | M | 72 | 26.698 | 1.938 | 30.921 | 1.00 | 20.94 | M | C |
| ATOM | 5391 | CB | THR | M | 72 | 27.284 | 0.506 | 30.771 | 1.00 | 15.01 | M | C |
| ATOM | 5392 | OG1 | THR | M | 72 | 27.724 | 0.299 | 29.424 | 1.00 | 20.94 | M | O |
| ATOM | 5393 | CG2 | THR | M | 72 | 26.246 | −0.530 | 31.127 | 1.00 | 28.76 | M | C |
| ATOM | 5394 | C | THR | M | 72 | 25.968 | 2.400 | 29.655 | 1.00 | 13.57 | M | C |
| ATOM | 5395 | O | THR | M | 72 | 24.786 | 2.116 | 29.460 | 1.00 | 22.05 | M | O |
| ATOM | 5396 | N | LEU | M | 73 | 26.655 | 3.228 | 28.878 | 1.00 | 13.40 | M | N |
| ATOM | 5397 | CA | LEU | M | 73 | 26.157 | 3.731 | 27.604 | 1.00 | 16.16 | M | C |
| ATOM | 5398 | CB | LEU | M | 73 | 26.476 | 5.223 | 27.422 | 1.00 | 16.87 | M | C |
| ATOM | 5399 | CG | LEU | M | 73 | 26.155 | 5.737 | 26.007 | 1.00 | 22.50 | M | C |
| ATOM | 5400 | CD1 | LEU | M | 73 | 24.635 | 5.723 | 25.741 | 1.00 | 18.84 | M | C |
| ATOM | 5401 | CD2 | LEU | M | 73 | 26.743 | 7.112 | 25.791 | 1.00 | 14.62 | M | C |
| ATOM | 5402 | C | LEU | M | 73 | 26.859 | 2.923 | 26.511 | 1.00 | 14.77 | M | C |
| ATOM | 5403 | O | LEU | M | 73 | 28.080 | 2.868 | 26.462 | 1.00 | 16.53 | M | O |
| ATOM | 5404 | N | THR | M | 74 | 26.077 | 2.257 | 25.670 | 1.00 | 25.78 | M | N |
| ATOM | 5405 | CA | THR | M | 74 | 26.623 | 1.453 | 24.587 | 1.00 | 22.71 | M | C |
| ATOM | 5406 | CB | THR | M | 74 | 26.090 | 0.017 | 24.647 | 1.00 | 23.30 | M | C |
| ATOM | 5407 | OG1 | THR | M | 74 | 26.440 | −0.558 | 25.912 | 1.00 | 23.75 | M | O |
| ATOM | 5408 | CG2 | THR | M | 74 | 26.701 | −0.840 | 23.540 | 1.00 | 8.73 | M | C |
| ATOM | 5409 | C | THR | M | 74 | 26.264 | 2.039 | 23.231 | 1.00 | 23.89 | M | C |
| ATOM | 5410 | O | THR | M | 74 | 25.148 | 2.515 | 23.048 | 1.00 | 25.01 | M | O |
| ATOM | 5411 | N | ILE | M | 75 | 27.259 | 2.119 | 22.341 | 1.00 | 26.22 | M | N |
| ATOM | 5412 | CA | ILE | M | 75 | 27.073 | 2.585 | 20.956 | 1.00 | 25.28 | M | C |
| ATOM | 5413 | CB | ILE | M | 75 | 28.018 | 3.742 | 20.566 | 1.00 | 24.04 | M | C |
| ATOM | 5414 | CG2 | ILE | M | 75 | 27.522 | 4.362 | 19.284 | 1.00 | 17.98 | M | C |
| ATOM | 5415 | CG1 | ILE | M | 75 | 28.055 | 4.814 | 21.662 | 1.00 | 21.10 | M | C |
| ATOM | 5416 | CD1 | ILE | M | 75 | 28.884 | 6.021 | 21.301 | 1.00 | 16.37 | M | C |
| ATOM | 5417 | C | ILE | M | 75 | 27.453 | 1.342 | 20.151 | 1.00 | 25.40 | M | C |
| ATOM | 5418 | O | ILE | M | 75 | 28.633 | 1.028 | 20.009 | 1.00 | 25.01 | M | O |
| ATOM | 5419 | N | SER | M | 76 | 26.449 | 0.624 | 19.655 | 1.00 | 28.94 | M | N |
| ATOM | 5420 | CA | SER | M | 76 | 26.678 | −0.634 | 18.947 | 1.00 | 33.89 | M | C |
| ATOM | 5421 | CB | SER | M | 76 | 25.388 | −1.452 | 18.890 | 1.00 | 30.88 | M | C |
| ATOM | 5422 | OG | SER | M | 76 | 24.535 | −1.005 | 17.846 | 1.00 | 44.83 | M | O |
| ATOM | 5423 | C | SER | M | 76 | 27.366 | −0.635 | 17.579 | 1.00 | 35.56 | M | C |
| ATOM | 5424 | O | SER | M | 76 | 27.955 | −1.648 | 17.184 | 1.00 | 43.18 | M | O |
| ATOM | 5425 | N | SER | M | 77 | 27.333 | 0.474 | 16.855 | 1.00 | 28.47 | M | N |
| ATOM | 5426 | CA | SER | M | 77 | 27.979 | 0.475 | 15.544 | 1.00 | 33.73 | M | C |
| ATOM | 5427 | CB | SER | M | 77 | 26.953 | 0.104 | 14.471 | 1.00 | 35.44 | M | C |
| ATOM | 5428 | OG | SER | M | 77 | 27.530 | −0.705 | 13.479 | 1.00 | 44.28 | M | O |
| ATOM | 5429 | C | SER | M | 77 | 28.475 | 1.888 | 15.337 | 1.00 | 34.11 | M | C |
| ATOM | 5430 | O | SER | M | 77 | 27.794 | 2.705 | 14.713 | 1.00 | 38.87 | M | O |
| ATOM | 5431 | N | LEU | M | 78 | 29.677 | 2.160 | 15.840 | 1.00 | 24.04 | M | N |
| ATOM | 5432 | CA | LEU | M | 78 | 30.228 | 3.502 | 15.782 | 1.00 | 20.57 | M | C |
| ATOM | 5433 | CB | LEU | M | 78 | 31.622 | 3.529 | 16.401 | 1.00 | 20.63 | M | C |
| ATOM | 5434 | CG | LEU | M | 78 | 32.160 | 4.909 | 16.739 | 1.00 | 18.36 | M | C |
| ATOM | 5435 | CD1 | LEU | M | 78 | 31.327 | 5.570 | 17.859 | 1.00 | 18.60 | M | C |
| ATOM | 5436 | CD2 | LEU | M | 78 | 33.596 | 4.735 | 17.179 | 1.00 | 17.48 | M | C |
| ATOM | 5437 | C | LEU | M | 78 | 30.253 | 4.136 | 14.407 | 1.00 | 18.85 | M | C |
| ATOM | 5438 | O | LEU | M | 78 | 30.861 | 3.612 | 13.482 | 1.00 | 23.34 | M | O |
| ATOM | 5439 | N | GLN | M | 79 | 29.586 | 5.278 | 14.295 | 1.00 | 17.12 | M | N |
| ATOM | 5440 | CA | GLN | M | 79 | 29.512 | 6.037 | 13.059 | 1.00 | 15.84 | M | C |
| ATOM | 5441 | CB | GLN | M | 79 | 28.060 | 6.468 | 12.828 | 1.00 | 19.92 | M | C |
| ATOM | 5442 | CG | GLN | M | 79 | 27.119 | 5.302 | 12.639 | 1.00 | 21.27 | M | C |
| ATOM | 5443 | CD | GLN | M | 79 | 27.562 | 4.433 | 11.489 | 1.00 | 16.75 | M | C |
| ATOM | 5444 | OE1 | GLN | M | 79 | 27.619 | 4.894 | 10.343 | 1.00 | 21.46 | M | O |
| ATOM | 5445 | NE2 | GLN | M | 79 | 27.918 | 3.180 | 11.785 | 1.00 | 17.40 | M | N |
| ATOM | 5446 | C | GLN | M | 79 | 30.393 | 7.296 | 13.109 | 1.00 | 18.91 | M | C |
| ATOM | 5447 | O | GLN | M | 79 | 30.675 | 7.814 | 14.185 | 1.00 | 22.47 | M | O |
| ATOM | 5448 | N | PRO | M | 80 | 30.807 | 7.820 | 11.938 | 1.00 | 19.06 | M | N |
| ATOM | 5449 | CD | PRO | M | 80 | 30.604 | 7.243 | 10.586 | 1.00 | 16.32 | M | C |
| ATOM | 5450 | CA | PRO | M | 80 | 31.638 | 9.027 | 11.871 | 1.00 | 15.66 | M | C |
| ATOM | 5451 | CB | PRO | M | 80 | 31.571 | 9.387 | 10.388 | 1.00 | 13.08 | M | C |
| ATOM | 5452 | CG | PRO | M | 80 | 31.574 | 8.035 | 9.726 | 1.00 | 8.00 | M | C |
| ATOM | 5453 | C | PRO | M | 80 | 31.073 | 10.172 | 12.724 | 1.00 | 21.75 | M | C |
| ATOM | 5454 | O | PRO | M | 80 | 31.817 | 10.875 | 13.427 | 1.00 | 30.62 | M | O |
| ATOM | 5455 | N | GLU | M | 81 | 29.755 | 10.344 | 12.670 | 1.00 | 17.97 | M | N |
| ATOM | 5456 | CA | GLU | M | 81 | 29.077 | 11.405 | 13.411 | 1.00 | 9.64 | M | C |
| ATOM | 5457 | CB | GLU | M | 81 | 27.663 | 11.661 | 12.869 | 1.00 | 11.37 | M | C |
| ATOM | 5458 | CG | GLU | M | 81 | 26.672 | 10.474 | 12.904 | 1.00 | 22.25 | M | C |
| ATOM | 5459 | CD | GLU | M | 81 | 26.625 | 9.643 | 11.577 | 1.00 | 31.92 | M | C |
| ATOM | 5460 | OE1 | GLU | M | 81 | 27.688 | 9.457 | 10.933 | 1.00 | 33.38 | M | O |
| ATOM | 5461 | OE2 | GLU | M | 81 | 25.521 | 9.168 | 11.191 | 1.00 | 29.70 | M | O |
| ATOM | 5462 | C | GLU | M | 81 | 29.030 | 11.137 | 14.905 | 1.00 | 18.36 | M | C |
| ATOM | 5463 | O | GLU | M | 81 | 28.657 | 12.010 | 15.686 | 1.00 | 17.82 | M | O |
| ATOM | 5464 | N | ASP | M | 82 | 29.453 | 9.945 | 15.310 | 1.00 | 17.12 | M | N |
| ATOM | 5465 | CA | ASP | M | 82 | 29.454 | 9.586 | 16.726 | 1.00 | 17.93 | M | C |
| ATOM | 5466 | CB | ASP | M | 82 | 29.237 | 8.078 | 16.880 | 1.00 | 12.02 | M | C |
| ATOM | 5467 | CG | ASP | M | 82 | 27.867 | 7.636 | 16.392 | 1.00 | 10.63 | M | C |
| ATOM | 5468 | OD1 | ASP | M | 82 | 26.975 | 8.489 | 16.256 | 1.00 | 12.33 | M | O |
| ATOM | 5469 | OD2 | ASP | M | 82 | 27.690 | 6.437 | 16.125 | 1.00 | 15.54 | M | O |
| ATOM | 5470 | C | ASP | M | 82 | 30.698 | 10.034 | 17.505 | 1.00 | 18.02 | M | C |
| ATOM | 5471 | O | ASP | M | 82 | 30.727 | 9.938 | 18.733 | 1.00 | 19.01 | M | O |
| ATOM | 5472 | N | PHE | M | 83 | 31.731 | 10.494 | 16.805 | 1.00 | 17.06 | M | N |
| ATOM | 5473 | CA | PHE | M | 83 | 32.937 | 10.970 | 17.489 | 1.00 | 18.81 | M | C |
| ATOM | 5474 | CB | PHE | M | 83 | 34.121 | 11.044 | 16.533 | 1.00 | 9.09 | M | C |
| ATOM | 5475 | CG | PHE | M | 83 | 34.546 | 9.713 | 16.025 | 1.00 | 13.65 | M | C |
| ATOM | 5476 | CD1 | PHE | M | 83 | 35.324 | 8.877 | 16.812 | 1.00 | 11.43 | M | C |
| ATOM | 5477 | CD2 | PHE | M | 83 | 34.154 | 9.278 | 14.756 | 1.00 | 19.70 | M | C |
| ATOM | 5478 | CE1 | PHE | M | 83 | 35.714 | 7.620 | 16.352 | 1.00 | 21.90 | M | C |
| ATOM | 5479 | CE2 | PHE | M | 83 | 34.535 | 8.023 | 14.279 | 1.00 | 18.70 | M | C |
| ATOM | 5480 | CZ | PHE | M | 83 | 35.314 | 7.189 | 15.069 | 1.00 | 25.54 | M | C |
| ATOM | 5481 | C | PHE | M | 83 | 32.627 | 12.323 | 18.109 | 1.00 | 18.80 | M | C |
| ATOM | 5482 | O | PHE | M | 83 | 32.355 | 13.317 | 17.417 | 1.00 | 17.10 | M | O |
| ATOM | 5483 | N | ALA | M | 84 | 32.653 | 12.336 | 19.432 | 1.00 | 17.96 | M | N |
| ATOM | 5484 | CA | ALA | M | 84 | 32.320 | 13.526 | 20.193 | 1.00 | 20.56 | M | C |
| ATOM | 5485 | CB | ALA | M | 84 | 30.835 | 13.834 | 19.995 | 1.00 | 19.49 | M | C |
| ATOM | 5486 | C | ALA | M | 84 | 32.591 | 13.222 | 21.652 | 1.00 | 16.59 | M | C |
| ATOM | 5487 | O | ALA | M | 84 | 33.292 | 12.249 | 21.964 | 1.00 | 9.51 | M | O |
| ATOM | 5488 | N | THR | M | 85 | 32.029 | 14.043 | 22.539 | 1.00 | 9.57 | M | N |
| ATOM | 5489 | CA | THR | M | 85 | 32.191 | 13.819 | 23.981 | 1.00 | 19.15 | M | C |
| ATOM | 5490 | CB | THR | M | 85 | 32.755 | 15.077 | 24.704 | 1.00 | 19.55 | M | C |
| ATOM | 5491 | OG1 | THR | M | 85 | 33.995 | 15.471 | 24.098 | 1.00 | 16.94 | M | O |
| ATOM | 5492 | CG2 | THR | M | 85 | 32.994 | 14.785 | 26.176 | 1.00 | 15.60 | M | C |
| ATOM | 5493 | C | THR | M | 85 | 30.839 | 13.396 | 24.607 | 1.00 | 19.43 | M | C |
| ATOM | 5494 | O | THR | M | 85 | 29.770 | 13.895 | 24.223 | 1.00 | 19.37 | M | O |
| ATOM | 5495 | N | TYR | M | 86 | 30.897 | 12.446 | 25.534 | 1.00 | 11.98 | M | N |
| ATOM | 5496 | CA | TYR | M | 86 | 29.700 | 11.931 | 26.203 | 1.00 | 17.41 | M | C |
| ATOM | 5497 | CB | TYR | M | 86 | 29.515 | 10.423 | 25.890 | 1.00 | 14.28 | M | C |
| ATOM | 5498 | CG | TYR | M | 86 | 29.257 | 10.165 | 24.409 | 1.00 | 21.01 | M | C |
| ATOM | 5499 | CD1 | TYR | M | 86 | 30.312 | 10.128 | 23.489 | 1.00 | 14.80 | M | C |
| ATOM | 5500 | CE1 | TYR | M | 86 | 30.083 | 9.993 | 22.143 | 1.00 | 14.14 | M | C |
| ATOM | 5501 | CD2 | TYR | M | 86 | 27.956 | 10.045 | 23.919 | 1.00 | 16.85 | M | C |
| ATOM | 5502 | CE2 | TYR | M | 86 | 27.715 | 9.905 | 22.567 | 1.00 | 17.21 | M | C |
| ATOM | 5503 | CZ | TYR | M | 86 | 28.775 | 9.883 | 21.678 | 1.00 | 22.33 | M | C |
| ATOM | 5504 | OH | TYR | M | 86 | 28.529 | 9.783 | 20.321 | 1.00 | 17.13 | M | O |
| ATOM | 5505 | C | TYR | M | 86 | 29.718 | 12.179 | 27.717 | 1.00 | 17.80 | M | C |
| ATOM | 5506 | O | TYR | M | 86 | 30.753 | 12.062 | 28.367 | 1.00 | 13.74 | M | O |
| ATOM | 5507 | N | TYR | M | 87 | 28.554 | 12.501 | 28.268 | 1.00 | 17.52 | M | N |
| ATOM | 5508 | CA | TYR | M | 87 | 28.434 | 12.773 | 29.692 | 1.00 | 16.30 | M | C |
| ATOM | 5509 | CB | TYR | M | 87 | 28.219 | 14.275 | 29.925 | 1.00 | 12.23 | M | C |
| ATOM | 5510 | CG | TYR | M | 87 | 29.337 | 15.189 | 29.502 | 1.00 | 12.33 | M | C |
| ATOM | 5511 | CD1 | TYR | M | 87 | 30.367 | 15.524 | 30.395 | 1.00 | 16.03 | M | C |
| ATOM | 5512 | CE1 | TYR | M | 87 | 31.353 | 16.429 | 30.046 | 1.00 | 5.16 | M | C |
| ATOM | 5513 | CD2 | TYR | M | 87 | 29.335 | 15.788 | 28.247 | 1.00 | 13.76 | M | C |
| ATOM | 5514 | CE2 | TYR | M | 87 | 30.303 | 16.692 | 27.894 | 1.00 | 8.34 | M | C |
| ATOM | 5515 | CZ | TYR | M | 87 | 31.317 | 17.010 | 28.805 | 1.00 | 12.96 | M | C |
| ATOM | 5516 | OH | TYR | M | 87 | 32.293 | 17.917 | 28.467 | 1.00 | 17.70 | M | O |
| ATOM | 5517 | C | TYR | M | 87 | 27.237 | 12.081 | 30.320 | 1.00 | 19.58 | M | C |
| ATOM | 5518 | O | TYR | M | 87 | 26.168 | 11.970 | 29.707 | 1.00 | 18.02 | M | O |
| ATOM | 5519 | N | CYS | M | 88 | 27.398 | 11.633 | 31.558 | 1.00 | 22.17 | M | N |
| ATOM | 5520 | CA | CYS | M | 88 | 26.259 | 11.067 | 32.263 | 1.00 | 23.03 | M | C |
| ATOM | 5521 | C | CYS | M | 88 | 25.759 | 12.193 | 33.175 | 1.00 | 22.31 | M | C |
| ATOM | 5522 | O | CYS | M | 88 | 26.503 | 13.120 | 33.473 | 1.00 | 20.44 | M | O |
| ATOM | 5523 | CB | CYS | M | 88 | 26.633 | 9.813 | 33.065 | 1.00 | 25.40 | M | C |
| ATOM | 5524 | SG | CYS | M | 88 | 28.191 | 9.845 | 34.002 | 1.00 | 30.37 | M | S |
| ATOM | 5525 | N | LEU | M | 89 | 24.467 | 12.191 | 33.477 | 1.00 | 22.16 | M | N |
| ATOM | 5526 | CA | LEU | M | 89 | 23.870 | 13.179 | 34.372 | 1.00 | 22.85 | M | C |
| ATOM | 5527 | CB | LEU | M | 89 | 22.929 | 14.120 | 33.624 | 1.00 | 24.58 | M | C |
| ATOM | 5528 | CG | LEU | M | 89 | 22.097 | 15.204 | 34.334 | 1.00 | 23.70 | M | C |
| ATOM | 5529 | CD1 | LEU | M | 89 | 21.097 | 14.574 | 35.304 | 1.00 | 20.65 | M | C |
| ATOM | 5530 | CD2 | LEU | M | 89 | 22.962 | 16.209 | 35.025 | 1.00 | 16.40 | M | C |
| ATOM | 5531 | C | LEU | M | 89 | 23.128 | 12.378 | 35.431 | 1.00 | 23.88 | M | C |
| ATOM | 5532 | O | LEU | M | 89 | 22.393 | 11.443 | 35.135 | 1.00 | 29.49 | M | O |
| ATOM | 5533 | N | GLN | M | 90 | 23.350 | 12.754 | 36.675 | 1.00 | 27.89 | M | N |
| ATOM | 5534 | CA | GLN | M | 90 | 22.770 | 12.093 | 37.823 | 1.00 | 18.65 | M | C |
| ATOM | 5535 | CB | GLN | M | 90 | 23.929 | 11.844 | 38.797 | 1.00 | 25.91 | M | C |
| ATOM | 5536 | CG | GLN | M | 90 | 23.607 | 11.531 | 40.226 | 1.00 | 23.12 | M | C |
| ATOM | 5537 | CD | GLN | M | 90 | 23.212 | 12.736 | 41.008 | 1.00 | 19.85 | M | C |
| ATOM | 5538 | OE1 | GLN | M | 90 | 22.101 | 12.806 | 41.498 | 1.00 | 27.15 | M | O |
| ATOM | 5539 | NE2 | GLN | M | 90 | 24.102 | 13.709 | 41.108 | 1.00 | 24.53 | M | N |
| ATOM | 5540 | C | GLN | M | 90 | 21.715 | 12.996 | 38.438 | 1.00 | 20.80 | M | C |
| ATOM | 5541 | O | GLN | M | 90 | 21.961 | 14.190 | 38.607 | 1.00 | 17.65 | M | O |
| ATOM | 5542 | N | HIS | M | 91 | 20.508 | 12.466 | 38.658 | 1.00 | 22.39 | M | N |
| ATOM | 5543 | CA | HIS | M | 91 | 19.464 | 13.242 | 39.316 | 1.00 | 18.96 | M | C |
| ATOM | 5544 | CB | HIS | M | 91 | 18.416 | 13.822 | 38.336 | 1.00 | 20.74 | M | C |
| ATOM | 5545 | CG | HIS | M | 91 | 17.490 | 12.814 | 37.733 | 1.00 | 25.28 | M | C |
| ATOM | 5546 | CD2 | HIS | M | 91 | 17.548 | 12.140 | 36.560 | 1.00 | 21.46 | M | C |
| ATOM | 5547 | ND1 | HIS | M | 91 | 16.319 | 12.418 | 38.346 | 1.00 | 19.14 | M | N |
| ATOM | 5548 | CE1 | HIS | M | 91 | 15.698 | 11.543 | 37.579 | 1.00 | 23.61 | M | C |
| ATOM | 5549 | NE2 | HIS | M | 91 | 16.422 | 11.354 | 36.490 | 1.00 | 25.39 | M | N |
| ATOM | 5550 | C | HIS | M | 91 | 18.854 | 12.498 | 40.515 | 1.00 | 17.31 | M | C |
| ATOM | 5551 | O | HIS | M | 91 | 17.764 | 12.815 | 40.974 | 1.00 | 23.49 | M | O |
| ATOM | 5552 | N | GLY | M | 92 | 19.642 | 11.595 | 41.094 | 1.00 | 14.82 | M | N |
| ATOM | 5553 | CA | GLY | M | 92 | 19.210 | 10.841 | 42.253 | 1.00 | 25.59 | M | C |
| ATOM | 5554 | C | GLY | M | 92 | 19.160 | 11.696 | 43.512 | 1.00 | 28.54 | M | C |
| ATOM | 5555 | O | GLY | M | 92 | 18.571 | 11.305 | 44.529 | 1.00 | 27.06 | M | O |
| ATOM | 5556 | N | GLU | M | 93 | 19.752 | 12.884 | 43.416 | 1.00 | 25.27 | M | N |
| ATOM | 5557 | CA | GLU | M | 93 | 19.804 | 13.842 | 44.512 | 1.00 | 26.18 | M | C |
| ATOM | 5558 | CB | GLU | M | 93 | 20.932 | 13.458 | 45.469 | 1.00 | 33.16 | M | C |
| ATOM | 5559 | CG | GLU | M | 93 | 21.292 | 14.512 | 46.506 | 1.00 | 40.27 | M | C |
| ATOM | 5560 | CD | GLU | M | 93 | 22.593 | 14.184 | 47.239 | 1.00 | 47.45 | M | C |
| ATOM | 5561 | OE1 | GLU | M | 93 | 23.693 | 14.409 | 46.662 | 1.00 | 39.75 | M | O |
| ATOM | 5562 | OE2 | GLU | M | 93 | 22.509 | 13.695 | 48.392 | 1.00 | 46.11 | M | O |
| ATOM | 5563 | C | GLU | M | 93 | 20.103 | 15.220 | 43.968 | 1.00 | 20.13 | M | C |
| ATOM | 5564 | O | GLU | M | 93 | 20.750 | 15.356 | 42.940 | 1.00 | 26.89 | M | O |
| ATOM | 5565 | N | SER | M | 94 | 19.573 | 16.245 | 44.611 | 1.00 | 15.33 | M | N |
| ATOM | 5566 | CA | SER | M | 94 | 19.902 | 17.608 | 44.198 | 1.00 | 16.52 | M | C |
| ATOM | 5567 | CB | SER | M | 94 | 18.773 | 18.565 | 44.502 | 1.00 | 16.95 | M | C |
| ATOM | 5568 | OG | SER | M | 94 | 19.141 | 19.887 | 44.177 | 1.00 | 27.99 | M | O |
| ATOM | 5569 | C | SER | M | 94 | 21.141 | 17.955 | 45.035 | 1.00 | 22.57 | M | C |
| ATOM | 5570 | O | SER | M | 94 | 21.189 | 17.647 | 46.236 | 1.00 | 21.56 | M | O |
| ATOM | 5571 | N | PRO | M | 95 | 22.151 | 18.615 | 44.430 | 1.00 | 22.62 | M | N |
| ATOM | 5572 | CD | PRO | M | 95 | 23.432 | 18.837 | 45.142 | 1.00 | 17.29 | M | C |
| ATOM | 5573 | CA | PRO | M | 95 | 22.250 | 19.046 | 43.029 | 1.00 | 14.71 | M | C |
| ATOM | 5574 | CB | PRO | M | 95 | 23.430 | 20.005 | 43.073 | 1.00 | 15.63 | M | C |
| ATOM | 5575 | CG | PRO | M | 95 | 24.369 | 19.289 | 44.034 | 1.00 | 19.40 | M | C |
| ATOM | 5576 | C | PRO | M | 95 | 22.531 | 17.925 | 42.034 | 1.00 | 14.75 | M | C |
| ATOM | 5577 | O | PRO | M | 95 | 23.141 | 16.924 | 42.381 | 1.00 | 12.84 | M | O |
| ATOM | 5578 | N | TRP | M | 96 | 22.084 | 18.117 | 40.794 | 1.00 | 10.51 | M | N |
| ATOM | 5579 | CA | TRP | M | 96 | 22.330 | 17.162 | 39.728 | 1.00 | 17.19 | M | C |
| ATOM | 5580 | CB | TRP | M | 96 | 21.422 | 17.447 | 38.508 | 1.00 | 17.50 | M | C |
| ATOM | 5581 | CG | TRP | M | 96 | 19.908 | 17.304 | 38.756 | 1.00 | 20.33 | M | C |
| ATOM | 5582 | CD2 | TRP | M | 96 | 18.863 | 17.354 | 37.770 | 1.00 | 14.01 | M | C |
| ATOM | 5583 | CE2 | TRP | M | 96 | 17.638 | 17.195 | 38.450 | 1.00 | 15.34 | M | C |
| ATOM | 5584 | CE3 | TRP | M | 96 | 18.847 | 17.520 | 36.377 | 1.00 | 11.36 | M | C |
| ATOM | 5585 | CD1 | TRP | M | 96 | 19.280 | 17.123 | 39.961 | 1.00 | 20.65 | M | C |
| ATOM | 5586 | NE1 | TRP | M | 96 | 17.920 | 17.056 | 39.782 | 1.00 | 16.85 | M | N |
| ATOM | 5587 | CZ2 | TRP | M | 96 | 16.408 | 17.192 | 37.787 | 1.00 | 10.87 | M | C |
| ATOM | 5588 | CZ3 | TRP | M | 96 | 17.638 | 17.519 | 35.725 | 1.00 | 12.44 | M | C |
| ATOM | 5589 | CH2 | TRP | M | 96 | 16.427 | 17.353 | 36.432 | 1.00 | 16.33 | M | C |
| ATOM | 5590 | C | TRP | M | 96 | 23.791 | 17.334 | 39.314 | 1.00 | 17.60 | M | C |
| ATOM | 5591 | O | TRP | M | 96 | 24.310 | 18.451 | 39.284 | 1.00 | 18.50 | M | O |
| ATOM | 5592 | N | THR | M | 97 | 24.477 | 16.235 | 39.039 | 1.00 | 16.37 | M | N |
| ATOM | 5593 | CA | THR | M | 97 | 25.864 | 16.338 | 38.621 | 1.00 | 12.81 | M | C |
| ATOM | 5594 | CB | THR | M | 97 | 26.870 | 15.923 | 39.719 | 1.00 | 18.87 | M | C |
| ATOM | 5595 | OG1 | THR | M | 97 | 26.635 | 14.566 | 40.097 | 1.00 | 20.10 | M | O |
| ATOM | 5596 | CG2 | THR | M | 97 | 26.787 | 16.847 | 40.950 | 1.00 | 12.34 | M | C |
| ATOM | 5597 | C | THR | M | 97 | 26.158 | 15.480 | 37.401 | 1.00 | 21.62 | M | C |
| ATOM | 5598 | O | THR | M | 97 | 25.561 | 14.419 | 37.192 | 1.00 | 20.60 | M | O |
| ATOM | 5599 | N | PHE | M | 98 | 27.119 | 15.950 | 36.620 | 1.00 | 19.46 | M | N |
| ATOM | 5600 | CA | PHE | M | 98 | 27.548 | 15.286 | 35.419 | 1.00 | 13.99 | M | C |
| ATOM | 5601 | CB | PHE | M | 98 | 27.833 | 16.313 | 34.320 | 1.00 | 12.97 | M | C |
| ATOM | 5602 | CG | PHE | M | 98 | 26.642 | 17.086 | 33.853 | 1.00 | 13.37 | M | C |
| ATOM | 5603 | CD1 | PHE | M | 98 | 26.274 | 18.270 | 34.479 | 1.00 | 10.56 | M | C |
| ATOM | 5604 | CD2 | PHE | M | 98 | 25.949 | 16.688 | 32.712 | 1.00 | 13.21 | M | C |
| ATOM | 5605 | CE1 | PHE | M | 98 | 25.238 | 19.053 | 33.970 | 1.00 | 14.38 | M | C |
| ATOM | 5606 | CE2 | PHE | M | 98 | 24.915 | 17.459 | 32.191 | 1.00 | 12.58 | M | C |
| ATOM | 5607 | CZ | PHE | M | 98 | 24.556 | 18.646 | 32.817 | 1.00 | 12.45 | M | C |
| ATOM | 5608 | C | PHE | M | 98 | 28.860 | 14.588 | 35.678 | 1.00 | 18.04 | M | C |
| ATOM | 5609 | O | PHE | M | 98 | 29.636 | 15.006 | 36.543 | 1.00 | 16.45 | M | O |
| ATOM | 5610 | N | GLY | M | 99 | 29.141 | 13.568 | 34.868 | 1.00 | 16.36 | M | N |
| ATOM | 5611 | CA | GLY | M | 99 | 30.420 | 12.885 | 34.970 | 1.00 | 12.86 | M | C |
| ATOM | 5612 | C | GLY | M | 99 | 31.443 | 13.815 | 34.332 | 1.00 | 7.48 | M | C |
| ATOM | 5613 | O | GLY | M | 99 | 31.081 | 14.819 | 33.719 | 1.00 | 20.38 | M | O |
| ATOM | 5614 | N | GLN | M | 100 | 32.717 | 13.483 | 34.444 | 1.00 | 18.14 | M | N |
| ATOM | 5615 | CA | GLN | M | 100 | 33.788 | 14.306 | 33.886 | 1.00 | 17.50 | M | C |
| ATOM | 5616 | CB | GLN | M | 100 | 35.143 | 13.812 | 34.385 | 1.00 | 21.41 | M | C |
| ATOM | 5617 | CG | GLN | M | 100 | 35.241 | 13.722 | 35.887 | 1.00 | 31.80 | M | C |
| ATOM | 5618 | CD | GLN | M | 100 | 34.432 | 12.570 | 36.486 | 1.00 | 35.31 | M | C |
| ATOM | 5619 | OE1 | GLN | M | 100 | 33.973 | 11.660 | 35.774 | 1.00 | 36.83 | M | O |
| ATOM | 5620 | NE2 | GLN | M | 100 | 34.263 | 12.603 | 37.809 | 1.00 | 37.84 | M | N |
| ATOM | 5621 | C | GLN | M | 100 | 33.800 | 14.320 | 32.368 | 1.00 | 15.07 | M | C |
| ATOM | 5622 | O | GLN | M | 100 | 34.352 | 15.229 | 31.781 | 1.00 | 18.71 | M | O |
| ATOM | 5623 | N | GLY | M | 101 | 33.237 | 13.293 | 31.737 | 1.00 | 21.63 | M | N |
| ATOM | 5624 | CA | GLY | M | 101 | 33.193 | 13.254 | 30.285 | 1.00 | 16.54 | M | C |
| ATOM | 5625 | C | GLY | M | 101 | 34.047 | 12.154 | 29.706 | 1.00 | 22.10 | M | C |
| ATOM | 5626 | O | GLY | M | 101 | 34.915 | 11.618 | 30.389 | 1.00 | 25.17 | M | O |
| ATOM | 5627 | N | THR | M | 102 | 33.723 | 11.749 | 28.482 | 1.00 | 20.59 | M | N |
| ATOM | 5628 | CA | THR | M | 102 | 34.464 | 10.719 | 27.766 | 1.00 | 15.63 | M | C |
| ATOM | 5629 | CB | THR | M | 102 | 33.787 | 9.326 | 27.782 | 1.00 | 18.49 | M | C |
| ATOM | 5630 | OG1 | THR | M | 102 | 33.792 | 8.782 | 29.105 | 1.00 | 24.11 | M | O |
| ATOM | 5631 | CG2 | THR | M | 102 | 34.522 | 8.360 | 26.850 | 1.00 | 15.27 | M | C |
| ATOM | 5632 | C | THR | M | 102 | 34.494 | 11.162 | 26.322 | 1.00 | 20.09 | M | C |
| ATOM | 5633 | O | THR | M | 102 | 33.445 | 11.252 | 25.678 | 1.00 | 21.57 | M | O |
| ATOM | 5634 | N | LYS | M | 103 | 35.690 | 11.488 | 25.838 | 1.00 | 15.81 | M | N |
| ATOM | 5635 | CA | LYS | M | 103 | 35.877 | 11.900 | 24.464 | 1.00 | 17.17 | M | C |
| ATOM | 5636 | CB | LYS | M | 103 | 37.019 | 12.910 | 24.351 | 1.00 | 20.89 | M | C |
| ATOM | 5637 | CG | LYS | M | 103 | 37.248 | 13.370 | 22.933 | 1.00 | 30.33 | M | C |
| ATOM | 5638 | CD | LYS | M | 103 | 38.236 | 14.515 | 22.824 | 1.00 | 29.07 | M | C |
| ATOM | 5639 | CE | LYS | M | 103 | 38.367 | 14.908 | 21.347 | 1.00 | 38.89 | M | C |
| ATOM | 5640 | NZ | LYS | M | 103 | 39.505 | 15.828 | 21.004 | 1.00 | 40.18 | M | N |
| ATOM | 5641 | C | LYS | M | 103 | 36.186 | 10.664 | 23.620 | 1.00 | 21.65 | M | C |
| ATOM | 5642 | O | LYS | M | 103 | 37.074 | 9.868 | 23.943 | 1.00 | 18.29 | M | O |
| ATOM | 5643 | N | VAL | M | 104 | 35.372 | 10.456 | 22.596 | 1.00 | 21.99 | M | N |
| ATOM | 5644 | CA | VAL | M | 104 | 35.555 | 9.341 | 21.692 | 1.00 | 15.70 | M | C |
| ATOM | 5645 | CB | VAL | M | 104 | 34.221 | 8.643 | 21.396 | 1.00 | 17.84 | M | C |
| ATOM | 5646 | CG1 | VAL | M | 104 | 34.378 | 7.637 | 20.238 | 1.00 | 13.29 | M | C |
| ATOM | 5647 | CG2 | VAL | M | 104 | 33.755 | 7.936 | 22.658 | 1.00 | 8.89 | M | C |
| ATOM | 5648 | C | VAL | M | 104 | 36.227 | 9.897 | 20.438 | 1.00 | 21.98 | M | C |
| ATOM | 5649 | O | VAL | M | 104 | 35.692 | 10.748 | 19.716 | 1.00 | 17.17 | M | O |
| ATOM | 5650 | N | GLU | M | 105 | 37.456 | 9.465 | 20.229 | 1.00 | 24.42 | M | N |
| ATOM | 5651 | CA | GLU | M | 105 | 38.212 | 9.958 | 19.104 | 1.00 | 24.13 | M | C |
| ATOM | 5652 | CB | GLU | M | 105 | 39.413 | 10.758 | 19.616 | 1.00 | 22.47 | M | C |
| ATOM | 5653 | CG | GLU | M | 105 | 40.292 | 10.031 | 20.589 | 1.00 | 31.86 | M | C |
| ATOM | 5654 | CD | GLU | M | 105 | 41.470 | 9.398 | 19.891 | 1.00 | 44.09 | M | C |
| ATOM | 5655 | OE1 | GLU | M | 105 | 42.399 | 10.147 | 19.510 | 1.00 | 49.02 | M | O |
| ATOM | 5656 | OE2 | GLU | M | 105 | 41.451 | 8.167 | 19.684 | 1.00 | 41.03 | M | O |
| ATOM | 5657 | C | GLU | M | 105 | 38.602 | 8.846 | 18.156 | 1.00 | 18.46 | M | C |
| ATOM | 5658 | O | GLU | M | 105 | 38.601 | 7.675 | 18.529 | 1.00 | 18.90 | M | O |
| ATOM | 5659 | N | ILE | M | 106 | 38.901 | 9.235 | 16.920 | 1.00 | 21.61 | M | N |
| ATOM | 5660 | CA | ILE | M | 106 | 39.263 | 8.316 | 15.842 | 1.00 | 21.67 | M | C |
| ATOM | 5661 | CB | ILE | M | 106 | 39.206 | 9.055 | 14.474 | 1.00 | 22.57 | M | C |
| ATOM | 5662 | CG2 | ILE | M | 106 | 39.129 | 8.057 | 13.320 | 1.00 | 18.74 | M | C |
| ATOM | 5663 | CG1 | ILE | M | 106 | 37.977 | 9.961 | 14.433 | 1.00 | 24.47 | M | C |
| ATOM | 5664 | CD1 | ILE | M | 106 | 37.927 | 10.875 | 13.238 | 1.00 | 26.99 | M | C |
| ATOM | 5665 | C | ILE | M | 106 | 40.618 | 7.629 | 15.973 | 1.00 | 18.99 | M | C |
| ATOM | 5666 | O | ILE | M | 106 | 41.648 | 8.288 | 16.075 | 1.00 | 19.38 | M | O |
| ATOM | 5667 | N | LYS | M | 107 | 40.604 | 6.302 | 15.941 | 1.00 | 21.59 | M | N |
| ATOM | 5668 | CA | LYS | M | 107 | 41.823 | 5.504 | 16.013 | 1.00 | 24.35 | M | C |
| ATOM | 5669 | CB | LYS | M | 107 | 41.484 | 4.075 | 16.443 | 1.00 | 27.11 | M | C |
| ATOM | 5670 | CG | LYS | M | 107 | 42.610 | 3.071 | 16.254 | 1.00 | 31.54 | M | C |
| ATOM | 5671 | CD | LYS | M | 107 | 43.317 | 2.711 | 17.565 | 1.00 | 38.80 | M | C |
| ATOM | 5672 | CE | LYS | M | 107 | 42.676 | 1.491 | 18.271 | 1.00 | 43.95 | M | C |
| ATOM | 5673 | NZ | LYS | M | 107 | 41.264 | 1.683 | 18.738 | 1.00 | 33.53 | M | N |
| ATOM | 5674 | C | LYS | M | 107 | 42.483 | 5.457 | 14.634 | 1.00 | 27.12 | M | C |
| ATOM | 5675 | O | LYS | M | 107 | 41.799 | 5.412 | 13.613 | 1.00 | 37.47 | M | O |
| ATOM | 5676 | N | ARG | M | 108 | 43.808 | 5.538 | 14.615 | 1.00 | 27.38 | M | N |
| ATOM | 5677 | CA | ARG | M | 108 | 44.599 | 5.449 | 13.388 | 1.00 | 25.76 | M | C |
| ATOM | 5678 | CB | ARG | M | 108 | 44.706 | 6.788 | 12.648 | 1.00 | 21.88 | M | C |
| ATOM | 5679 | CG | ARG | M | 108 | 45.216 | 7.960 | 13.464 | 1.00 | 26.28 | M | C |
| ATOM | 5680 | CD | ARG | M | 108 | 46.019 | 8.927 | 12.590 | 1.00 | 24.82 | M | C |
| ATOM | 5681 | NE | ARG | M | 108 | 47.270 | 8.309 | 12.150 | 1.00 | 25.04 | M | N |
| ATOM | 5682 | CZ | ARG | M | 108 | 47.910 | 8.616 | 11.025 | 1.00 | 23.41 | M | C |
| ATOM | 5683 | NH1 | ARG | M | 108 | 47.437 | 9.548 | 10.206 | 1.00 | 11.67 | M | N |
| ATOM | 5684 | NH2 | ARG | M | 108 | 49.012 | 7.958 | 10.700 | 1.00 | 22.37 | M | N |
| ATOM | 5685 | C | ARG | M | 108 | 45.986 | 4.921 | 13.771 | 1.00 | 28.44 | M | C |
| ATOM | 5686 | O | ARG | M | 108 | 46.239 | 4.617 | 14.945 | 1.00 | 29.66 | M | O |
| ATOM | 5687 | N | THR | M | 109 | 46.866 | 4.742 | 12.791 | 1.00 | 25.88 | M | N |
| ATOM | 5688 | CA | THR | M | 109 | 48.203 | 4.241 | 13.112 | 1.00 | 26.03 | M | C |
| ATOM | 5689 | CB | THR | M | 109 | 48.978 | 3.842 | 11.861 | 1.00 | 27.91 | M | C |
| ATOM | 5690 | OG1 | THR | M | 109 | 48.997 | 4.948 | 10.944 | 1.00 | 24.50 | M | O |
| ATOM | 5691 | CG2 | THR | M | 109 | 48.338 | 2.606 | 11.205 | 1.00 | 25.65 | M | C |
| ATOM | 5692 | C | THR | M | 109 | 49.025 | 5.293 | 13.845 | 1.00 | 27.31 | M | C |
| ATOM | 5693 | O | THR | M | 109 | 48.789 | 6.503 | 13.704 | 1.00 | 23.93 | M | O |
| ATOM | 5694 | N | VAL | M | 110 | 49.964 | 4.811 | 14.649 | 1.00 | 27.43 | M | N |
| ATOM | 5695 | CA | VAL | M | 110 | 50.865 | 5.667 | 15.396 | 1.00 | 27.12 | M | C |
| ATOM | 5696 | CB | VAL | M | 110 | 51.818 | 4.826 | 16.246 | 1.00 | 24.73 | M | C |
| ATOM | 5697 | CG1 | VAL | M | 110 | 52.928 | 5.698 | 16.822 | 1.00 | 21.86 | M | C |
| ATOM | 5698 | CG2 | VAL | M | 110 | 51.032 | 4.148 | 17.351 | 1.00 | 18.04 | M | C |
| ATOM | 5699 | C | VAL | M | 110 | 51.669 | 6.526 | 14.423 | 1.00 | 29.54 | M | C |
| ATOM | 5700 | O | VAL | M | 110 | 52.222 | 6.018 | 13.451 | 1.00 | 36.77 | M | O |
| ATOM | 5701 | N | ALA | M | 111 | 51.659 | 7.834 | 14.657 | 1.00 | 27.01 | M | N |
| ATOM | 5702 | CA | ALA | M | 111 | 52.383 | 8.769 | 13.819 | 1.00 | 23.66 | M | C |
| ATOM | 5703 | CB | ALA | M | 111 | 51.422 | 9.579 | 12.971 | 1.00 | 19.17 | M | C |
| ATOM | 5704 | C | ALA | M | 111 | 53.234 | 9.678 | 14.720 | 1.00 | 31.52 | M | C |
| ATOM | 5705 | O | ALA | M | 111 | 52.733 | 10.341 | 15.638 | 1.00 | 29.98 | M | O |
| ATOM | 5706 | N | ALA | M | 112 | 54.540 | 9.625 | 14.487 | 1.00 | 29.04 | M | N |
| ATOM | 5707 | CA | ALA | M | 112 | 55.508 | 10.403 | 15.231 | 1.00 | 27.20 | M | C |
| ATOM | 5708 | CB | ALA | M | 112 | 56.900 | 9.887 | 14.942 | 1.00 | 22.64 | M | C |
| ATOM | 5709 | C | ALA | M | 112 | 55.430 | 11.888 | 14.883 | 1.00 | 27.29 | M | C |
| ATOM | 5710 | O | ALA | M | 112 | 55.255 | 12.270 | 13.717 | 1.00 | 25.88 | M | O |
| ATOM | 5711 | N | PRO | M | 113 | 55.601 | 12.750 | 15.892 | 1.00 | 24.76 | M | N |
| ATOM | 5712 | CD | PRO | M | 113 | 55.878 | 12.460 | 17.311 | 1.00 | 19.79 | M | C |
| ATOM | 5713 | CA | PRO | M | 113 | 55.547 | 14.189 | 15.631 | 1.00 | 25.47 | M | C |
| ATOM | 5714 | CB | PRO | M | 113 | 55.441 | 14.782 | 17.032 | 1.00 | 18.06 | M | C |
| ATOM | 5715 | CG | PRO | M | 113 | 56.284 | 13.814 | 17.848 | 1.00 | 27.32 | M | C |
| ATOM | 5716 | C | PRO | M | 113 | 56.821 | 14.684 | 14.965 | 1.00 | 25.08 | M | C |
| ATOM | 5717 | O | PRO | M | 113 | 57.890 | 14.104 | 15.148 | 1.00 | 27.44 | M | O |
| ATOM | 5718 | N | SER | M | 114 | 56.690 | 15.682 | 14.102 | 1.00 | 25.82 | M | N |
| ATOM | 5719 | CA | SER | M | 114 | 57.873 | 16.294 | 13.528 | 1.00 | 24.76 | M | C |
| ATOM | 5720 | CB | SER | M | 114 | 57.649 | 16.667 | 12.068 | 1.00 | 17.25 | M | C |
| ATOM | 5721 | OG | SER | M | 114 | 56.499 | 17.470 | 11.926 | 1.00 | 40.47 | M | O |
| ATOM | 5722 | C | SER | M | 114 | 58.007 | 17.520 | 14.466 | 1.00 | 26.96 | M | C |
| ATOM | 5723 | O | SER | M | 114 | 57.055 | 18.281 | 14.657 | 1.00 | 22.83 | M | O |
| ATOM | 5724 | N | VAL | M | 115 | 59.139 | 17.606 | 15.157 | 1.00 | 27.55 | M | N |
| ATOM | 5725 | CA | VAL | M | 115 | 59.407 | 18.669 | 16.130 | 1.00 | 25.41 | M | C |
| ATOM | 5726 | CB | VAL | M | 115 | 60.086 | 18.053 | 17.368 | 1.00 | 21.75 | M | C |
| ATOM | 5727 | CG1 | VAL | M | 115 | 60.166 | 19.055 | 18.490 | 1.00 | 16.79 | M | C |
| ATOM | 5728 | CG2 | VAL | M | 115 | 59.336 | 16.818 | 17.795 | 1.00 | 15.23 | M | C |
| ATOM | 5729 | C | VAL | M | 115 | 60.239 | 19.865 | 15.630 | 1.00 | 25.17 | M | C |
| ATOM | 5730 | O | VAL | M | 115 | 61.238 | 19.689 | 14.945 | 1.00 | 23.79 | M | O |
| ATOM | 5731 | N | PHE | M | 116 | 59.838 | 21.075 | 16.023 | 1.00 | 28.68 | M | N |
| ATOM | 5732 | CA | PHE | M | 116 | 60.519 | 22.313 | 15.631 | 1.00 | 16.37 | M | C |
| ATOM | 5733 | CB | PHE | M | 116 | 59.736 | 23.052 | 14.539 | 1.00 | 24.46 | M | C |
| ATOM | 5734 | CG | PHE | M | 116 | 59.600 | 22.295 | 13.243 | 1.00 | 28.23 | M | C |
| ATOM | 5735 | CD1 | PHE | M | 116 | 60.531 | 22.458 | 12.230 | 1.00 | 25.94 | M | C |
| ATOM | 5736 | CD2 | PHE | M | 116 | 58.529 | 21.422 | 13.032 | 1.00 | 31.38 | M | C |
| ATOM | 5737 | CE1 | PHE | M | 116 | 60.403 | 21.757 | 11.013 | 1.00 | 34.69 | M | C |
| ATOM | 5738 | CE2 | PHE | M | 116 | 58.392 | 20.717 | 11.820 | 1.00 | 31.35 | M | C |
| ATOM | 5739 | CZ | PHE | M | 116 | 59.336 | 20.888 | 10.809 | 1.00 | 23.11 | M | C |
| ATOM | 5740 | C | PHE | M | 116 | 60.593 | 23.256 | 16.827 | 1.00 | 27.96 | M | C |
| ATOM | 5741 | O | PHE | M | 116 | 59.644 | 23.320 | 17.619 | 1.00 | 24.59 | M | O |
| ATOM | 5742 | N | ILE | M | 117 | 61.699 | 24.006 | 16.942 | 1.00 | 26.18 | M | N |
| ATOM | 5743 | CA | ILE | M | 117 | 61.872 | 24.988 | 18.018 | 1.00 | 16.60 | M | C |
| ATOM | 5744 | CB | ILE | M | 117 | 63.121 | 24.698 | 18.883 | 1.00 | 29.61 | M | C |
| ATOM | 5745 | CG2 | ILE | M | 117 | 64.411 | 24.900 | 18.079 | 1.00 | 24.33 | M | C |
| ATOM | 5746 | CG1 | ILE | M | 117 | 63.169 | 25.640 | 20.089 | 1.00 | 27.16 | M | C |
| ATOM | 5747 | CD1 | ILE | M | 117 | 62.612 | 25.049 | 21.332 | 1.00 | 37.41 | M | C |
| ATOM | 5748 | C | ILE | M | 117 | 61.988 | 26.390 | 17.418 | 1.00 | 22.28 | M | C |
| ATOM | 5749 | O | ILE | M | 117 | 62.605 | 26.570 | 16.371 | 1.00 | 25.54 | M | O |
| ATOM | 5750 | N | PHE | M | 118 | 61.355 | 27.377 | 18.046 | 1.00 | 20.92 | M | N |
| ATOM | 5751 | CA | PHE | M | 118 | 61.433 | 28.746 | 17.558 | 1.00 | 19.88 | M | C |
| ATOM | 5752 | CB | PHE | M | 118 | 60.074 | 29.231 | 17.053 | 1.00 | 22.70 | M | C |
| ATOM | 5753 | CG | PHE | M | 118 | 59.537 | 28.446 | 15.897 | 1.00 | 25.00 | M | C |
| ATOM | 5754 | CD1 | PHE | M | 118 | 59.943 | 28.733 | 14.594 | 1.00 | 22.62 | M | C |
| ATOM | 5755 | CD2 | PHE | M | 118 | 58.642 | 27.394 | 16.108 | 1.00 | 23.92 | M | C |
| ATOM | 5756 | CE1 | PHE | M | 118 | 59.460 | 27.975 | 13.514 | 1.00 | 27.19 | M | C |
| ATOM | 5757 | CE2 | PHE | M | 118 | 58.153 | 26.625 | 15.032 | 1.00 | 22.19 | M | C |
| ATOM | 5758 | CZ | PHE | M | 118 | 58.563 | 26.914 | 13.732 | 1.00 | 19.67 | M | C |
| ATOM | 5759 | C | PHE | M | 118 | 61.933 | 29.687 | 18.657 | 1.00 | 28.45 | M | C |
| ATOM | 5760 | O | PHE | M | 118 | 61.393 | 29.714 | 19.766 | 1.00 | 32.43 | M | O |
| ATOM | 5761 | N | PRO | M | 119 | 63.017 | 30.428 | 18.384 | 1.00 | 29.50 | M | N |
| ATOM | 5762 | CD | PRO | M | 119 | 63.973 | 30.259 | 17.270 | 1.00 | 32.07 | M | C |
| ATOM | 5763 | CA | PRO | M | 119 | 63.533 | 31.350 | 19.399 | 1.00 | 30.35 | M | C |
| ATOM | 5764 | CB | PRO | M | 119 | 64.994 | 31.550 | 18.975 | 1.00 | 30.20 | M | C |
| ATOM | 5765 | CG | PRO | M | 119 | 64.951 | 31.400 | 17.488 | 1.00 | 32.34 | M | C |
| ATOM | 5766 | C | PRO | M | 119 | 62.757 | 32.655 | 19.385 | 1.00 | 27.81 | M | C |
| ATOM | 5767 | O | PRO | M | 119 | 62.015 | 32.934 | 18.451 | 1.00 | 24.85 | M | O |
| ATOM | 5768 | N | PRO | M | 120 | 62.885 | 33.457 | 20.450 | 1.00 | 25.68 | M | N |
| ATOM | 5769 | CD | PRO | M | 120 | 63.638 | 33.176 | 21.681 | 1.00 | 21.72 | M | C |
| ATOM | 5770 | CA | PRO | M | 120 | 62.187 | 34.741 | 20.533 | 1.00 | 23.99 | M | C |
| ATOM | 5771 | CB | PRO | M | 120 | 62.693 | 35.305 | 21.846 | 1.00 | 21.95 | M | C |
| ATOM | 5772 | CG | PRO | M | 120 | 63.000 | 34.089 | 22.660 | 1.00 | 23.44 | M | C |
| ATOM | 5773 | C | PRO | M | 120 | 62.651 | 35.632 | 19.386 | 1.00 | 27.81 | M | C |
| ATOM | 5774 | O | PRO | M | 120 | 63.833 | 35.637 | 19.049 | 1.00 | 31.47 | M | O |
| ATOM | 5775 | N | SER | M | 121 | 61.726 | 36.344 | 18.758 | 1.00 | 28.87 | M | N |
| ATOM | 5776 | CA | SER | M | 121 | 62.087 | 37.256 | 17.671 | 1.00 | 34.12 | M | C |
| ATOM | 5777 | CB | SER | M | 121 | 60.834 | 37.688 | 16.905 | 1.00 | 34.87 | M | C |
| ATOM | 5778 | OG | SER | M | 121 | 59.937 | 38.391 | 17.758 | 1.00 | 32.43 | M | O |
| ATOM | 5779 | C | SER | M | 121 | 62.752 | 38.495 | 18.280 | 1.00 | 36.11 | M | C |
| ATOM | 5780 | O | SER | M | 121 | 62.492 | 38.831 | 19.445 | 1.00 | 36.68 | M | O |
| ATOM | 5781 | N | ASP | M | 122 | 63.585 | 39.180 | 17.496 | 1.00 | 37.09 | M | N |
| ATOM | 5782 | CA | ASP | M | 122 | 64.263 | 40.388 | 17.974 | 1.00 | 38.62 | M | C |
| ATOM | 5783 | CB | ASP | M | 122 | 65.304 | 40.892 | 16.953 | 1.00 | 42.24 | M | C |
| ATOM | 5784 | CG | ASP | M | 122 | 66.466 | 39.903 | 16.740 | 1.00 | 48.31 | M | C |
| ATOM | 5785 | OD1 | ASP | M | 122 | 66.894 | 39.237 | 17.710 | 1.00 | 46.06 | M | O |
| ATOM | 5786 | OD2 | ASP | M | 122 | 66.963 | 39.795 | 15.593 | 1.00 | 51.42 | M | O |
| ATOM | 5787 | C | ASP | M | 122 | 63.252 | 41.497 | 18.316 | 1.00 | 39.11 | M | C |
| ATOM | 5788 | O | ASP | M | 122 | 63.471 | 42.285 | 19.247 | 1.00 | 37.61 | M | O |
| ATOM | 5789 | N | GLU | M | 123 | 62.126 | 41.519 | 17.604 | 1.00 | 37.70 | M | N |
| ATOM | 5790 | CA | GLU | M | 123 | 61.076 | 42.515 | 17.841 | 1.00 | 40.68 | M | C |
| ATOM | 5791 | CB | GLU | M | 123 | 60.019 | 42.443 | 16.726 | 1.00 | 49.41 | M | C |
| ATOM | 5792 | CG | GLU | M | 123 | 59.109 | 43.669 | 16.610 | 1.00 | 55.60 | M | C |
| ATOM | 5793 | CD | GLU | M | 123 | 57.913 | 43.470 | 15.659 | 1.00 | 62.27 | M | C |
| ATOM | 5794 | OE1 | GLU | M | 123 | 57.880 | 42.486 | 14.869 | 1.00 | 60.81 | M | O |
| ATOM | 5795 | OE2 | GLU | M | 123 | 56.990 | 44.315 | 15.713 | 1.00 | 64.31 | M | O |
| ATOM | 5796 | C | GLU | M | 123 | 60.422 | 42.332 | 19.226 | 1.00 | 38.93 | M | C |
| ATOM | 5797 | O | GLU | M | 123 | 60.044 | 43.316 | 19.868 | 1.00 | 39.81 | M | O |
| ATOM | 5798 | N | GLN | M | 124 | 60.287 | 41.084 | 19.684 | 1.00 | 35.60 | M | N |
| ATOM | 5799 | CA | GLN | M | 124 | 59.694 | 40.812 | 21.001 | 1.00 | 35.54 | M | C |
| ATOM | 5800 | CB | GLN | M | 124 | 59.211 | 39.359 | 21.116 | 1.00 | 30.91 | M | C |
| ATOM | 5801 | CG | GLN | M | 124 | 58.605 | 39.036 | 22.479 | 1.00 | 30.43 | M | C |
| ATOM | 5802 | CD | GLN | M | 124 | 58.198 | 37.584 | 22.658 | 1.00 | 32.83 | M | C |
| ATOM | 5803 | OE1 | GLN | M | 124 | 58.711 | 36.684 | 21.982 | 1.00 | 33.20 | M | O |
| ATOM | 5804 | NE2 | GLN | M | 124 | 57.268 | 37.346 | 23.584 | 1.00 | 31.44 | M | N |
| ATOM | 5805 | C | GLN | M | 124 | 60.675 | 41.114 | 22.142 | 1.00 | 32.88 | M | C |
| ATOM | 5806 | O | GLN | M | 124 | 60.283 | 41.647 | 23.186 | 1.00 | 31.89 | M | O |
| ATOM | 5807 | N | LEU | M | 125 | 61.939 | 40.733 | 21.958 | 1.00 | 34.25 | M | N |
| ATOM | 5808 | CA | LEU | M | 125 | 62.977 | 40.981 | 22.966 | 1.00 | 32.38 | M | C |
| ATOM | 5809 | CB | LEU | M | 125 | 64.332 | 40.468 | 22.491 | 1.00 | 24.24 | M | C |
| ATOM | 5810 | CG | LEU | M | 125 | 64.418 | 38.943 | 22.539 | 1.00 | 26.01 | M | C |
| ATOM | 5811 | CD1 | LEU | M | 125 | 65.653 | 38.439 | 21.815 | 1.00 | 23.38 | M | C |
| ATOM | 5812 | CD2 | LEU | M | 125 | 64.398 | 38.481 | 23.993 | 1.00 | 21.81 | M | C |
| ATOM | 5813 | C | LEU | M | 125 | 63.052 | 42.471 | 23.256 | 1.00 | 29.00 | M | C |
| ATOM | 5814 | O | LEU | M | 125 | 63.164 | 42.879 | 24.402 | 1.00 | 33.85 | M | O |
| ATOM | 5815 | N | LYS | M | 126 | 62.868 | 43.268 | 22.212 | 1.00 | 28.95 | M | N |
| ATOM | 5816 | CA | LYS | M | 126 | 62.881 | 44.722 | 22.307 | 1.00 | 34.53 | M | C |
| ATOM | 5817 | CB | LYS | M | 126 | 62.698 | 45.308 | 20.910 | 1.00 | 40.35 | M | C |
| ATOM | 5818 | CG | LYS | M | 126 | 63.453 | 46.580 | 20.632 | 1.00 | 48.24 | M | C |
| ATOM | 5819 | CD | LYS | M | 126 | 64.440 | 46.371 | 19.485 | 1.00 | 54.68 | M | C |
| ATOM | 5820 | CE | LYS | M | 126 | 63.791 | 45.728 | 18.243 | 1.00 | 55.97 | M | C |
| ATOM | 5821 | NZ | LYS | M | 126 | 62.608 | 46.477 | 17.705 | 1.00 | 55.66 | M | N |
| ATOM | 5822 | C | LYS | M | 126 | 61.777 | 45.256 | 23.238 | 1.00 | 36.20 | M | C |
| ATOM | 5823 | O | LYS | M | 126 | 61.874 | 46.371 | 23.746 | 1.00 | 40.43 | M | O |
| ATOM | 5824 | N | SER | M | 127 | 60.717 | 44.482 | 23.444 | 1.00 | 29.62 | M | N |
| ATOM | 5825 | CA | SER | M | 127 | 59.650 | 44.925 | 24.324 | 1.00 | 31.76 | M | C |
| ATOM | 5826 | CB | SER | M | 127 | 58.283 | 44.519 | 23.767 | 1.00 | 31.04 | M | C |
| ATOM | 5827 | OG | SER | M | 127 | 58.036 | 43.137 | 23.972 | 1.00 | 37.37 | M | O |
| ATOM | 5828 | C | SER | M | 127 | 59.817 | 44.397 | 25.754 | 1.00 | 30.76 | M | C |
| ATOM | 5829 | O | SER | M | 127 | 58.934 | 44.583 | 26.595 | 1.00 | 30.78 | M | O |
| ATOM | 5830 | N | GLY | M | 128 | 60.922 | 43.703 | 26.017 | 1.00 | 25.14 | M | N |
| ATOM | 5831 | CA | GLY | M | 128 | 61.159 | 43.180 | 27.355 | 1.00 | 21.20 | M | C |
| ATOM | 5832 | C | GLY | M | 128 | 60.757 | 41.751 | 27.732 | 1.00 | 27.21 | M | C |
| ATOM | 5833 | O | GLY | M | 128 | 61.015 | 41.327 | 28.861 | 1.00 | 28.07 | M | O |
| ATOM | 5834 | N | THR | M | 129 | 60.123 | 41.005 | 26.833 | 1.00 | 18.04 | M | N |
| ATOM | 5835 | CA | THR | M | 129 | 59.729 | 39.638 | 27.150 | 1.00 | 25.63 | M | C |
| ATOM | 5836 | CB | THR | M | 129 | 58.185 | 39.468 | 27.173 | 1.00 | 23.26 | M | C |
| ATOM | 5837 | OG1 | THR | M | 129 | 57.644 | 39.910 | 25.922 | 1.00 | 33.91 | M | O |
| ATOM | 5838 | CG2 | THR | M | 129 | 57.565 | 40.300 | 28.267 | 1.00 | 27.52 | M | C |
| ATOM | 5839 | C | THR | M | 129 | 60.308 | 38.698 | 26.109 | 1.00 | 23.90 | M | C |
| ATOM | 5840 | O | THR | M | 129 | 60.790 | 39.137 | 25.070 | 1.00 | 27.90 | M | O |
| ATOM | 5841 | N | ALA | M | 130 | 60.265 | 37.406 | 26.398 | 1.00 | 24.20 | M | N |
| ATOM | 5842 | CA | ALA | M | 130 | 60.771 | 36.415 | 25.467 | 1.00 | 22.84 | M | C |
| ATOM | 5843 | CB | ALA | M | 130 | 62.217 | 36.083 | 25.785 | 1.00 | 27.96 | M | C |
| ATOM | 5844 | C | ALA | M | 130 | 59.951 | 35.156 | 25.529 | 1.00 | 18.90 | M | C |
| ATOM | 5845 | O | ALA | M | 130 | 59.766 | 34.589 | 26.601 | 1.00 | 25.67 | M | O |
| ATOM | 5846 | N | SER | M | 131 | 59.414 | 34.732 | 24.394 | 1.00 | 24.15 | M | N |
| ATOM | 5847 | CA | SER | M | 131 | 58.680 | 33.479 | 24.397 | 1.00 | 25.94 | M | C |
| ATOM | 5848 | CB | SER | M | 131 | 57.149 | 33.667 | 24.304 | 1.00 | 27.31 | M | C |
| ATOM | 5849 | OG | SER | M | 131 | 56.727 | 34.373 | 23.164 | 1.00 | 26.64 | M | O |
| ATOM | 5850 | C | SER | M | 131 | 59.260 | 32.516 | 23.376 | 1.00 | 25.24 | M | C |
| ATOM | 5851 | O | SER | M | 131 | 59.554 | 32.892 | 22.242 | 1.00 | 23.76 | M | O |
| ATOM | 5852 | N | VAL | M | 132 | 59.588 | 31.323 | 23.858 | 1.00 | 19.60 | M | N |
| ATOM | 5853 | CA | VAL | M | 132 | 60.153 | 30.278 | 23.016 | 1.00 | 25.12 | M | C |
| ATOM | 5854 | CB | VAL | M | 132 | 61.344 | 29.573 | 23.714 | 1.00 | 29.14 | M | C |
| ATOM | 5855 | CG1 | VAL | M | 132 | 62.141 | 28.776 | 22.702 | 1.00 | 26.28 | M | C |
| ATOM | 5856 | CG2 | VAL | M | 132 | 62.240 | 30.602 | 24.391 | 1.00 | 30.44 | M | C |
| ATOM | 5857 | C | VAL | M | 132 | 59.051 | 29.260 | 22.688 | 1.00 | 27.26 | M | C |
| ATOM | 5858 | O | VAL | M | 132 | 58.370 | 28.734 | 23.581 | 1.00 | 21.90 | M | O |
| ATOM | 5859 | N | VAL | M | 133 | 58.877 | 28.980 | 21.405 | 1.00 | 23.10 | M | N |
| ATOM | 5860 | CA | VAL | M | 133 | 57.838 | 28.054 | 21.006 | 1.00 | 26.53 | M | C |
| ATOM | 5861 | CB | VAL | M | 133 | 56.866 | 28.737 | 20.038 | 1.00 | 25.50 | M | C |
| ATOM | 5862 | CG1 | VAL | M | 133 | 55.894 | 27.730 | 19.449 | 1.00 | 25.94 | M | C |
| ATOM | 5863 | CG2 | VAL | M | 133 | 56.109 | 29.833 | 20.777 | 1.00 | 21.64 | M | C |
| ATOM | 5864 | C | VAL | M | 133 | 58.312 | 26.715 | 20.445 | 1.00 | 24.96 | M | C |
| ATOM | 5865 | O | VAL | M | 133 | 59.173 | 26.668 | 19.571 | 1.00 | 28.58 | M | O |
| ATOM | 5866 | N | CYS | M | 134 | 57.775 | 25.634 | 21.009 | 1.00 | 24.87 | M | N |
| ATOM | 5867 | CA | CYS | M | 134 | 58.082 | 24.273 | 20.572 | 1.00 | 31.36 | M | C |
| ATOM | 5868 | C | CYS | M | 134 | 56.837 | 23.731 | 19.873 | 1.00 | 32.24 | M | C |
| ATOM | 5869 | O | CYS | M | 134 | 55.745 | 23.766 | 20.433 | 1.00 | 35.17 | M | O |
| ATOM | 5870 | CB | CYS | M | 134 | 58.431 | 23.380 | 21.757 | 1.00 | 30.86 | M | C |
| ATOM | 5871 | SG | CYS | M | 134 | 59.039 | 21.717 | 21.314 | 1.00 | 39.24 | M | S |
| ATOM | 5872 | N | LEU | M | 135 | 57.013 | 23.254 | 18.643 | 1.00 | 30.59 | M | N |
| ATOM | 5873 | CA | LEU | M | 135 | 55.929 | 22.712 | 17.827 | 1.00 | 22.88 | M | C |
| ATOM | 5874 | CB | LEU | M | 135 | 55.914 | 23.403 | 16.459 | 1.00 | 24.51 | M | C |
| ATOM | 5875 | CG | LEU | M | 135 | 55.008 | 22.821 | 15.364 | 1.00 | 29.74 | M | C |
| ATOM | 5876 | CD1 | LEU | M | 135 | 53.524 | 22.808 | 15.793 | 1.00 | 29.15 | M | C |
| ATOM | 5877 | CD2 | LEU | M | 135 | 55.169 | 23.646 | 14.108 | 1.00 | 20.72 | M | C |
| ATOM | 5878 | C | LEU | M | 135 | 56.033 | 21.216 | 17.597 | 1.00 | 27.49 | M | C |
| ATOM | 5879 | O | LEU | M | 135 | 57.094 | 20.713 | 17.204 | 1.00 | 31.17 | M | O |
| ATOM | 5880 | N | LEU | M | 136 | 54.945 | 20.509 | 17.901 | 1.00 | 23.00 | M | N |
| ATOM | 5881 | CA | LEU | M | 136 | 54.831 | 19.065 | 17.675 | 1.00 | 16.36 | M | C |
| ATOM | 5882 | CB | LEU | M | 136 | 54.307 | 18.322 | 18.901 | 1.00 | 13.97 | M | C |
| ATOM | 5883 | CG | LEU | M | 136 | 55.334 | 17.901 | 19.952 | 1.00 | 19.79 | M | C |
| ATOM | 5884 | CD1 | LEU | M | 136 | 56.008 | 19.126 | 20.531 | 1.00 | 19.03 | M | C |
| ATOM | 5885 | CD2 | LEU | M | 136 | 54.671 | 17.111 | 21.036 | 1.00 | 14.67 | M | C |
| ATOM | 5886 | C | LEU | M | 136 | 53.790 | 18.992 | 16.566 | 1.00 | 22.08 | M | C |
| ATOM | 5887 | O | LEU | M | 136 | 52.603 | 19.259 | 16.792 | 1.00 | 23.21 | M | O |
| ATOM | 5888 | N | ASN | M | 137 | 54.244 | 18.669 | 15.362 | 1.00 | 22.76 | M | N |
| ATOM | 5889 | CA | ASN | M | 137 | 53.367 | 18.609 | 14.207 | 1.00 | 27.01 | M | C |
| ATOM | 5890 | CB | ASN | M | 137 | 54.030 | 19.314 | 13.030 | 1.00 | 25.09 | M | C |
| ATOM | 5891 | CG | ASN | M | 137 | 53.035 | 19.812 | 12.022 | 1.00 | 30.76 | M | C |
| ATOM | 5892 | OD1 | ASN | M | 137 | 52.142 | 20.586 | 12.352 | 1.00 | 34.56 | M | O |
| ATOM | 5893 | ND2 | ASN | M | 137 | 53.187 | 19.383 | 10.776 | 1.00 | 32.63 | M | N |
| ATOM | 5894 | C | ASN | M | 137 | 52.885 | 17.227 | 13.767 | 1.00 | 29.94 | M | C |
| ATOM | 5895 | O | ASN | M | 137 | 53.666 | 16.272 | 13.683 | 1.00 | 24.35 | M | O |
| ATOM | 5896 | N | ASN | M | 138 | 51.575 | 17.160 | 13.513 | 1.00 | 28.15 | M | N |
| ATOM | 5897 | CA | ASN | M | 138 | 50.873 | 15.987 | 13.021 | 1.00 | 26.36 | M | C |
| ATOM | 5898 | CB | ASN | M | 138 | 51.018 | 15.917 | 11.499 | 1.00 | 29.49 | M | C |
| ATOM | 5899 | CG | ASN | M | 138 | 50.331 | 17.060 | 10.781 | 1.00 | 28.77 | M | C |
| ATOM | 5900 | OD1 | ASN | M | 138 | 50.104 | 18.122 | 11.340 | 1.00 | 29.38 | M | O |
| ATOM | 5901 | ND2 | ASN | M | 138 | 49.991 | 16.834 | 9.528 | 1.00 | 41.75 | M | N |
| ATOM | 5902 | C | ASN | M | 138 | 51.260 | 14.642 | 13.609 | 1.00 | 30.92 | M | C |
| ATOM | 5903 | O | ASN | M | 138 | 51.835 | 13.804 | 12.904 | 1.00 | 34.09 | M | O |
| ATOM | 5904 | N | PHE | M | 139 | 50.885 | 14.397 | 14.861 | 1.00 | 28.28 | M | N |
| ATOM | 5905 | CA | PHE | M | 139 | 51.202 | 13.126 | 15.511 | 1.00 | 19.82 | M | C |
| ATOM | 5906 | CB | PHE | M | 139 | 52.201 | 13.372 | 16.646 | 1.00 | 17.78 | M | C |
| ATOM | 5907 | CG | PHE | M | 139 | 51.654 | 14.235 | 17.766 | 1.00 | 18.66 | M | C |
| ATOM | 5908 | CD1 | PHE | M | 139 | 51.682 | 15.623 | 17.677 | 1.00 | 14.91 | M | C |
| ATOM | 5909 | CD2 | PHE | M | 139 | 51.077 | 13.658 | 18.886 | 1.00 | 15.94 | M | C |
| ATOM | 5910 | CE1 | PHE | M | 139 | 51.141 | 16.412 | 18.682 | 1.00 | 16.59 | M | C |
| ATOM | 5911 | CE2 | PHE | M | 139 | 50.531 | 14.445 | 19.895 | 1.00 | 16.08 | M | C |
| ATOM | 5912 | CZ | PHE | M | 139 | 50.562 | 15.822 | 19.793 | 1.00 | 8.67 | M | C |
| ATOM | 5913 | C | PHE | M | 139 | 49.960 | 12.363 | 16.044 | 1.00 | 21.58 | M | C |
| ATOM | 5914 | O | PHE | M | 139 | 48.861 | 12.914 | 16.160 | 1.00 | 23.85 | M | O |
| ATOM | 5915 | N | TYR | M | 140 | 50.155 | 11.085 | 16.346 | 1.00 | 16.32 | M | N |
| ATOM | 5916 | CA | TYR | M | 140 | 49.116 | 10.231 | 16.889 | 1.00 | 19.69 | M | C |
| ATOM | 5917 | CB | TYR | M | 140 | 48.229 | 9.645 | 15.776 | 1.00 | 17.33 | M | C |
| ATOM | 5918 | CG | TYR | M | 140 | 47.029 | 8.929 | 16.358 | 1.00 | 17.42 | M | C |
| ATOM | 5919 | CD1 | TYR | M | 140 | 47.102 | 7.573 | 16.704 | 1.00 | 14.68 | M | C |
| ATOM | 5920 | CE1 | TYR | M | 140 | 46.046 | 6.935 | 17.320 | 1.00 | 17.41 | M | C |
| ATOM | 5921 | CD2 | TYR | M | 140 | 45.842 | 9.626 | 16.647 | 1.00 | 18.43 | M | C |
| ATOM | 5922 | CE2 | TYR | M | 140 | 44.762 | 8.984 | 17.275 | 1.00 | 14.93 | M | C |
| ATOM | 5923 | CZ | TYR | M | 140 | 44.881 | 7.647 | 17.605 | 1.00 | 13.64 | M | C |
| ATOM | 5924 | OH | TYR | M | 140 | 43.861 | 7.008 | 18.251 | 1.00 | 27.46 | M | O |
| ATOM | 5925 | C | TYR | M | 140 | 49.802 | 9.079 | 17.609 | 1.00 | 20.58 | M | C |
| ATOM | 5926 | O | TYR | M | 140 | 50.749 | 8.513 | 17.069 | 1.00 | 29.17 | M | O |
| ATOM | 5927 | N | PRO | M | 141 | 49.336 | 8.702 | 18.821 | 1.00 | 21.99 | M | N |
| ATOM | 5928 | CD | PRO | M | 141 | 49.962 | 7.548 | 19.501 | 1.00 | 21.18 | M | C |
| ATOM | 5929 | CA | PRO | M | 141 | 48.190 | 9.197 | 19.599 | 1.00 | 24.11 | M | C |
| ATOM | 5930 | CB | PRO | M | 141 | 48.007 | 8.112 | 20.658 | 1.00 | 25.23 | M | C |
| ATOM | 5931 | CG | PRO | M | 141 | 49.421 | 7.636 | 20.905 | 1.00 | 16.72 | M | C |
| ATOM | 5932 | C | PRO | M | 141 | 48.398 | 10.578 | 20.245 | 1.00 | 25.76 | M | C |
| ATOM | 5933 | O | PRO | M | 141 | 49.460 | 11.187 | 20.122 | 1.00 | 28.71 | M | O |
| ATOM | 5934 | N | ARG | M | 142 | 47.374 | 11.030 | 20.965 | 1.00 | 29.65 | M | N |
| ATOM | 5935 | CA | ARG | M | 142 | 47.366 | 12.334 | 21.628 | 1.00 | 32.42 | M | C |
| ATOM | 5936 | CB | ARG | M | 142 | 45.969 | 12.609 | 22.206 | 1.00 | 35.12 | M | C |
| ATOM | 5937 | CG | ARG | M | 142 | 45.614 | 14.087 | 22.401 | 1.00 | 36.60 | M | C |
| ATOM | 5938 | CD | ARG | M | 142 | 46.508 | 14.711 | 23.420 | 1.00 | 43.60 | M | C |
| ATOM | 5939 | NE | ARG | M | 142 | 46.002 | 15.944 | 23.992 | 1.00 | 47.54 | M | N |
| ATOM | 5940 | CZ | ARG | M | 142 | 46.758 | 16.775 | 24.701 | 1.00 | 48.85 | M | C |
| ATOM | 5941 | NH1 | ARG | M | 142 | 48.049 | 16.502 | 24.897 | 1.00 | 50.46 | M | N |
| ATOM | 5942 | NH2 | ARG | M | 142 | 46.213 | 17.837 | 25.273 | 1.00 | 50.37 | M | N |
| ATOM | 5943 | C | ARG | M | 142 | 48.440 | 12.528 | 22.707 | 1.00 | 34.31 | M | C |
| ATOM | 5944 | O | ARG | M | 142 | 49.099 | 13.571 | 22.726 | 1.00 | 36.83 | M | O |
| ATOM | 5945 | N | GLU | M | 143 | 48.612 | 11.550 | 23.596 | 1.00 | 28.38 | M | N |
| ATOM | 5946 | CA | GLU | M | 143 | 49.605 | 11.671 | 24.651 | 1.00 | 37.06 | M | C |
| ATOM | 5947 | CB | GLU | M | 143 | 49.649 | 10.413 | 25.521 | 1.00 | 45.09 | M | C |
| ATOM | 5948 | CG | GLU | M | 143 | 50.678 | 10.518 | 26.655 | 1.00 | 59.95 | M | C |
| ATOM | 5949 | CD | GLU | M | 143 | 50.792 | 9.260 | 27.496 | 1.00 | 65.68 | M | C |
| ATOM | 5950 | OE1 | GLU | M | 143 | 49.785 | 8.878 | 28.136 | 1.00 | 71.64 | M | O |
| ATOM | 5951 | OE2 | GLU | M | 143 | 51.896 | 8.665 | 27.529 | 1.00 | 66.73 | M | O |
| ATOM | 5952 | C | GLU | M | 143 | 51.019 | 12.035 | 24.154 | 1.00 | 37.14 | M | C |
| ATOM | 5953 | O | GLU | M | 143 | 51.600 | 11.363 | 23.301 | 1.00 | 38.90 | M | O |
| ATOM | 5954 | N | ALA | M | 144 | 51.548 | 13.126 | 24.693 | 1.00 | 35.91 | M | N |
| ATOM | 5955 | CA | ALA | M | 144 | 52.873 | 13.612 | 24.334 | 1.00 | 38.51 | M | C |
| ATOM | 5956 | CB | ALA | M | 144 | 52.779 | 14.548 | 23.119 | 1.00 | 27.54 | M | C |
| ATOM | 5957 | C | ALA | M | 144 | 53.465 | 14.357 | 25.527 | 1.00 | 35.57 | M | C |
| ATOM | 5958 | O | ALA | M | 144 | 52.768 | 15.124 | 26.183 | 1.00 | 35.85 | M | O |
| ATOM | 5959 | N | LYS | M | 145 | 54.721 | 14.063 | 25.861 | 1.00 | 40.92 | M | N |
| ATOM | 5960 | CA | LYS | M | 145 | 55.388 | 14.754 | 26.962 | 1.00 | 38.42 | M | C |
| ATOM | 5961 | CB | LYS | M | 145 | 56.119 | 13.798 | 27.905 | 1.00 | 42.88 | M | C |
| ATOM | 5962 | CG | LYS | M | 145 | 56.768 | 14.545 | 29.093 | 1.00 | 49.47 | M | C |
| ATOM | 5963 | CD | LYS | M | 145 | 57.263 | 13.628 | 30.225 | 1.00 | 50.08 | M | C |
| ATOM | 5964 | CE | LYS | M | 145 | 58.633 | 13.026 | 29.933 | 1.00 | 53.85 | M | C |
| ATOM | 5965 | NZ | LYS | M | 145 | 59.175 | 12.253 | 31.097 | 1.00 | 55.73 | M | N |
| ATOM | 5966 | C | LYS | M | 145 | 56.375 | 15.754 | 26.392 | 1.00 | 32.89 | M | C |
| ATOM | 5967 | O | LYS | M | 145 | 57.269 | 15.392 | 25.625 | 1.00 | 29.18 | M | O |
| ATOM | 5968 | N | VAL | M | 146 | 56.143 | 17.024 | 26.697 | 1.00 | 29.25 | M | N |
| ATOM | 5969 | CA | VAL | M | 146 | 57.023 | 18.086 | 26.239 | 1.00 | 33.78 | M | C |
| ATOM | 5970 | CB | VAL | M | 146 | 56.280 | 19.120 | 25.399 | 1.00 | 30.09 | M | C |
| ATOM | 5971 | CG1 | VAL | M | 146 | 57.247 | 20.119 | 24.829 | 1.00 | 39.11 | M | C |
| ATOM | 5972 | CG2 | VAL | M | 146 | 55.545 | 18.434 | 24.282 | 1.00 | 43.82 | M | C |
| ATOM | 5973 | C | VAL | M | 146 | 57.646 | 18.768 | 27.452 | 1.00 | 37.37 | M | C |
| ATOM | 5974 | O | VAL | M | 146 | 56.945 | 19.379 | 28.273 | 1.00 | 34.56 | M | O |
| ATOM | 5975 | N | GLN | M | 147 | 58.953 | 18.577 | 27.608 | 1.00 | 35.69 | M | N |
| ATOM | 5976 | CA | GLN | M | 147 | 59.679 | 19.187 | 28.712 | 1.00 | 39.29 | M | C |
| ATOM | 5977 | CB | GLN | M | 147 | 60.431 | 18.139 | 29.551 | 1.00 | 41.64 | M | C |
| ATOM | 5978 | CG | GLN | M | 147 | 59.556 | 17.384 | 30.558 | 1.00 | 49.60 | M | C |
| ATOM | 5979 | CD | GLN | M | 147 | 60.325 | 16.335 | 31.360 | 1.00 | 54.02 | M | C |
| ATOM | 5980 | OE1 | GLN | M | 147 | 60.055 | 16.121 | 32.543 | 1.00 | 51.92 | M | O |
| ATOM | 5981 | NE2 | GLN | M | 147 | 61.283 | 15.672 | 30.713 | 1.00 | 55.41 | M | N |
| ATOM | 5982 | C | GLN | M | 147 | 60.652 | 20.216 | 28.178 | 1.00 | 34.16 | M | C |
| ATOM | 5983 | O | GLN | M | 147 | 61.267 | 20.030 | 27.127 | 1.00 | 34.47 | M | O |
| ATOM | 5984 | N | TRP | M | 148 | 60.747 | 21.329 | 28.886 | 1.00 | 32.21 | M | N |
| ATOM | 5985 | CA | TRP | M | 148 | 61.658 | 22.391 | 28.514 | 1.00 | 27.83 | M | C |
| ATOM | 5986 | CB | TRP | M | 148 | 60.999 | 23.750 | 28.745 | 1.00 | 26.13 | M | C |
| ATOM | 5987 | CG | TRP | M | 148 | 60.026 | 24.131 | 27.691 | 1.00 | 26.70 | M | C |
| ATOM | 5988 | CD2 | TRP | M | 148 | 60.330 | 24.695 | 26.411 | 1.00 | 27.40 | M | C |
| ATOM | 5989 | CE2 | TRP | M | 148 | 59.097 | 24.923 | 25.749 | 1.00 | 29.98 | M | C |
| ATOM | 5990 | CE3 | TRP | M | 148 | 61.524 | 25.027 | 25.752 | 1.00 | 30.10 | M | C |
| ATOM | 5991 | CD1 | TRP | M | 148 | 58.659 | 24.038 | 27.755 | 1.00 | 28.15 | M | C |
| ATOM | 5992 | NE1 | TRP | M | 148 | 58.096 | 24.518 | 26.592 | 1.00 | 27.29 | M | N |
| ATOM | 5993 | CZ2 | TRP | M | 148 | 59.025 | 25.476 | 24.464 | 1.00 | 31.38 | M | C |
| ATOM | 5994 | CZ3 | TRP | M | 148 | 61.454 | 25.579 | 24.470 | 1.00 | 26.64 | M | C |
| ATOM | 5995 | CH2 | TRP | M | 148 | 60.215 | 25.794 | 23.842 | 1.00 | 32.25 | M | C |
| ATOM | 5996 | C | TRP | M | 148 | 62.963 | 22.272 | 29.313 | 1.00 | 30.06 | M | C |
| ATOM | 5997 | O | TRP | M | 148 | 62.956 | 21.997 | 30.518 | 1.00 | 25.46 | M | O |
| ATOM | 5998 | N | LYS | M | 149 | 64.087 | 22.456 | 28.634 | 1.00 | 32.34 | M | N |
| ATOM | 5999 | CA | LYS | M | 149 | 65.391 | 22.381 | 29.288 | 1.00 | 31.35 | M | C |
| ATOM | 6000 | CB | LYS | M | 149 | 66.152 | 21.139 | 28.831 | 1.00 | 37.11 | M | C |
| ATOM | 6001 | CG | LYS | M | 149 | 65.640 | 19.819 | 29.405 | 1.00 | 43.39 | M | C |
| ATOM | 6002 | CD | LYS | M | 149 | 66.412 | 18.638 | 28.793 | 1.00 | 45.27 | M | C |
| ATOM | 6003 | CE | LYS | M | 149 | 66.413 | 17.399 | 29.698 | 1.00 | 47.36 | M | C |
| ATOM | 6004 | NZ | LYS | M | 149 | 65.085 | 16.755 | 29.879 | 1.00 | 41.31 | M | N |
| ATOM | 6005 | C | LYS | M | 149 | 66.187 | 23.622 | 28.950 | 1.00 | 27.04 | M | C |
| ATOM | 6006 | O | LYS | M | 149 | 66.402 | 23.926 | 27.781 | 1.00 | 37.26 | M | O |
| ATOM | 6007 | N | VAL | M | 150 | 66.568 | 24.373 | 29.975 | 1.00 | 31.21 | M | N |
| ATOM | 6008 | CA | VAL | M | 150 | 67.341 | 25.607 | 29.803 | 1.00 | 32.33 | M | C |
| ATOM | 6009 | CB | VAL | M | 150 | 66.570 | 26.803 | 30.454 | 1.00 | 31.51 | M | C |
| ATOM | 6010 | CG1 | VAL | M | 150 | 67.346 | 28.113 | 30.340 | 1.00 | 26.39 | M | C |
| ATOM | 6011 | CG2 | VAL | M | 150 | 65.220 | 26.953 | 29.773 | 1.00 | 30.75 | M | C |
| ATOM | 6012 | C | VAL | M | 150 | 68.695 | 25.336 | 30.471 | 1.00 | 29.58 | M | C |
| ATOM | 6013 | O | VAL | M | 150 | 68.766 | 25.238 | 31.683 | 1.00 | 34.00 | M | O |
| ATOM | 6014 | N | ASP | M | 151 | 69.751 | 25.185 | 29.667 | 1.00 | 39.06 | M | N |
| ATOM | 6015 | CA | ASP | M | 151 | 71.106 | 24.844 | 30.168 | 1.00 | 40.76 | M | C |
| ATOM | 6016 | CB | ASP | M | 151 | 71.709 | 25.942 | 31.074 | 1.00 | 41.13 | M | C |
| ATOM | 6017 | CG | ASP | M | 151 | 72.308 | 27.104 | 30.297 | 1.00 | 42.08 | M | C |
| ATOM | 6018 | OD1 | ASP | M | 151 | 72.557 | 26.966 | 29.079 | 1.00 | 42.90 | M | O |
| ATOM | 6019 | OD2 | ASP | M | 151 | 72.538 | 28.164 | 30.919 | 1.00 | 45.56 | M | O |
| ATOM | 6020 | C | ASP | M | 151 | 70.985 | 23.544 | 30.965 | 1.00 | 40.80 | M | C |
| ATOM | 6021 | O | ASP | M | 151 | 71.468 | 23.445 | 32.099 | 1.00 | 40.49 | M | O |
| ATOM | 6022 | N | ASN | M | 152 | 70.261 | 22.584 | 30.390 | 1.00 | 39.88 | M | N |
| ATOM | 6023 | CA | ASN | M | 152 | 70.016 | 21.290 | 31.016 | 1.00 | 42.82 | M | C |
| ATOM | 6024 | CB | ASN | M | 152 | 71.308 | 20.510 | 31.215 | 1.00 | 42.30 | M | C |
| ATOM | 6025 | CG | ASN | M | 152 | 71.826 | 19.949 | 29.924 | 1.00 | 47.27 | M | C |
| ATOM | 6026 | OD1 | ASN | M | 152 | 72.460 | 20.658 | 29.138 | 1.00 | 46.62 | M | O |
| ATOM | 6027 | ND2 | ASN | M | 152 | 71.509 | 18.681 | 29.661 | 1.00 | 50.44 | M | N |
| ATOM | 6028 | C | ASN | M | 152 | 69.209 | 21.324 | 32.308 | 1.00 | 44.70 | M | C |
| ATOM | 6029 | O | ASN | M | 152 | 69.128 | 20.316 | 33.015 | 1.00 | 49.81 | M | O |
| ATOM | 6030 | N | ALA | M | 153 | 68.631 | 22.479 | 32.630 | 1.00 | 40.50 | M | N |
| ATOM | 6031 | CA | ALA | M | 153 | 67.799 | 22.590 | 33.823 | 1.00 | 40.03 | M | C |
| ATOM | 6032 | CB | ALA | M | 153 | 68.036 | 23.921 | 34.556 | 1.00 | 33.43 | M | C |
| ATOM | 6033 | C | ALA | M | 153 | 66.326 | 22.453 | 33.401 | 1.00 | 42.24 | M | C |
| ATOM | 6034 | O | ALA | M | 153 | 65.846 | 23.155 | 32.505 | 1.00 | 33.81 | M | O |
| ATOM | 6035 | N | LEU | M | 154 | 65.641 | 21.514 | 34.047 | 1.00 | 44.18 | M | N |
| ATOM | 6036 | CA | LEU | M | 154 | 64.240 | 21.215 | 33.810 | 1.00 | 42.74 | M | C |
| ATOM | 6037 | CB | LEU | M | 154 | 63.882 | 19.947 | 34.587 | 1.00 | 48.65 | M | C |
| ATOM | 6038 | CG | LEU | M | 154 | 62.797 | 18.979 | 34.108 | 1.00 | 51.94 | M | C |
| ATOM | 6039 | CD1 | LEU | M | 154 | 63.209 | 18.390 | 32.765 | 1.00 | 55.39 | M | C |
| ATOM | 6040 | CD2 | LEU | M | 154 | 62.615 | 17.856 | 35.141 | 1.00 | 53.29 | M | C |
| ATOM | 6041 | C | LEU | M | 154 | 63.358 | 22.373 | 34.289 | 1.00 | 44.99 | M | C |
| ATOM | 6042 | O | LEU | M | 154 | 63.407 | 22.761 | 35.463 | 1.00 | 37.76 | M | O |
| ATOM | 6043 | N | GLN | M | 155 | 62.540 | 22.912 | 33.386 | 1.00 | 42.07 | M | N |
| ATOM | 6044 | CA | GLN | M | 155 | 61.651 | 24.021 | 33.736 | 1.00 | 40.30 | M | C |
| ATOM | 6045 | CB | GLN | M | 155 | 61.451 | 24.949 | 32.541 | 1.00 | 39.49 | M | C |
| ATOM | 6046 | CG | GLN | M | 155 | 62.745 | 25.438 | 31.921 | 1.00 | 47.57 | M | C |
| ATOM | 6047 | CD | GLN | M | 155 | 63.521 | 26.387 | 32.821 | 1.00 | 47.33 | M | C |
| ATOM | 6048 | OE1 | GLN | M | 155 | 63.125 | 27.538 | 33.005 | 1.00 | 47.37 | M | O |
| ATOM | 6049 | NE2 | GLN | M | 155 | 64.652 | 25.920 | 33.353 | 1.00 | 46.55 | M | N |
| ATOM | 6050 | C | GLN | M | 155 | 60.295 | 23.535 | 34.221 | 1.00 | 38.40 | M | C |
| ATOM | 6051 | O | GLN | M | 155 | 59.894 | 22.400 | 33.943 | 1.00 | 43.13 | M | O |
| ATOM | 6052 | N | SER | M | 156 | 59.614 | 24.388 | 34.979 | 1.00 | 37.69 | M | N |
| ATOM | 6053 | CA | SER | M | 156 | 58.283 | 24.086 | 35.493 | 1.00 | 39.81 | M | C |
| ATOM | 6054 | CB | SER | M | 156 | 58.342 | 23.150 | 36.708 | 1.00 | 42.42 | M | C |
| ATOM | 6055 | OG | SER | M | 156 | 58.897 | 23.804 | 37.836 | 1.00 | 52.55 | M | O |
| ATOM | 6056 | C | SER | M | 156 | 57.549 | 25.379 | 35.843 | 1.00 | 39.66 | M | C |
| ATOM | 6057 | O | SER | M | 156 | 58.157 | 26.352 | 36.301 | 1.00 | 41.62 | M | O |
| ATOM | 6058 | N | GLY | M | 157 | 56.251 | 25.396 | 35.548 | 1.00 | 38.98 | M | N |
| ATOM | 6059 | CA | GLY | M | 157 | 55.413 | 26.546 | 35.840 | 1.00 | 31.21 | M | C |
| ATOM | 6060 | C | GLY | M | 157 | 55.501 | 27.730 | 34.900 | 1.00 | 30.02 | M | C |
| ATOM | 6061 | O | GLY | M | 157 | 54.738 | 28.672 | 35.042 | 1.00 | 36.09 | M | O |
| ATOM | 6062 | N | ASN | M | 158 | 56.407 | 27.695 | 33.930 | 1.00 | 31.36 | M | N |
| ATOM | 6063 | CA | ASN | M | 158 | 56.555 | 28.820 | 33.003 | 1.00 | 28.65 | M | C |
| ATOM | 6064 | CB | ASN | M | 158 | 57.976 | 29.392 | 33.094 | 1.00 | 29.75 | M | C |
| ATOM | 6065 | CG | ASN | M | 158 | 59.049 | 28.339 | 32.838 | 1.00 | 30.34 | M | C |
| ATOM | 6066 | OD1 | ASN | M | 158 | 58.744 | 27.211 | 32.461 | 1.00 | 36.91 | M | O |
| ATOM | 6067 | ND2 | ASN | M | 158 | 60.310 | 28.706 | 33.050 | 1.00 | 34.61 | M | N |
| ATOM | 6068 | C | ASN | M | 158 | 56.197 | 28.498 | 31.542 | 1.00 | 26.62 | M | C |
| ATOM | 6069 | O | ASN | M | 158 | 56.566 | 29.230 | 30.616 | 1.00 | 22.55 | M | O |
| ATOM | 6070 | N | SER | M | 159 | 55.512 | 27.380 | 31.336 | 1.00 | 23.85 | M | N |
| ATOM | 6071 | CA | SER | M | 159 | 55.088 | 27.001 | 29.991 | 1.00 | 29.65 | M | C |
| ATOM | 6072 | CB | SER | M | 159 | 55.918 | 25.817 | 29.471 | 1.00 | 25.62 | M | C |
| ATOM | 6073 | OG | SER | M | 159 | 55.941 | 24.748 | 30.393 | 1.00 | 28.93 | M | O |
| ATOM | 6074 | C | SER | M | 159 | 53.589 | 26.685 | 29.925 | 1.00 | 26.91 | M | C |
| ATOM | 6075 | O | SER | M | 159 | 52.955 | 26.392 | 30.940 | 1.00 | 32.68 | M | O |
| ATOM | 6076 | N | GLN | M | 160 | 53.023 | 26.817 | 28.734 | 1.00 | 27.80 | M | N |
| ATOM | 6077 | CA | GLN | M | 160 | 51.609 | 26.514 | 28.483 | 1.00 | 29.11 | M | C |
| ATOM | 6078 | CB | GLN | M | 160 | 50.757 | 27.788 | 28.444 | 1.00 | 23.07 | M | C |
| ATOM | 6079 | CG | GLN | M | 160 | 50.399 | 28.275 | 29.817 | 1.00 | 32.39 | M | C |
| ATOM | 6080 | CD | GLN | M | 160 | 49.515 | 29.495 | 29.791 | 1.00 | 38.22 | M | C |
| ATOM | 6081 | OE1 | GLN | M | 160 | 49.876 | 30.528 | 29.221 | 1.00 | 38.73 | M | O |
| ATOM | 6082 | NE2 | GLN | M | 160 | 48.337 | 29.386 | 30.410 | 1.00 | 41.30 | M | N |
| ATOM | 6083 | C | GLN | M | 160 | 51.475 | 25.764 | 27.164 | 1.00 | 25.08 | M | C |
| ATOM | 6084 | O | GLN | M | 160 | 52.150 | 26.086 | 26.193 | 1.00 | 21.96 | M | O |
| ATOM | 6085 | N | GLU | M | 161 | 50.640 | 24.731 | 27.159 | 1.00 | 26.20 | M | N |
| ATOM | 6086 | CA | GLU | M | 161 | 50.410 | 23.917 | 25.973 | 1.00 | 24.79 | M | C |
| ATOM | 6087 | CB | GLU | M | 161 | 50.425 | 22.441 | 26.341 | 1.00 | 23.66 | M | C |
| ATOM | 6088 | CG | GLU | M | 161 | 51.744 | 21.907 | 26.794 | 1.00 | 32.88 | M | C |
| ATOM | 6089 | CD | GLU | M | 161 | 51.794 | 20.389 | 26.788 | 1.00 | 35.81 | M | C |
| ATOM | 6090 | OE1 | GLU | M | 161 | 50.723 | 19.736 | 26.870 | 1.00 | 36.46 | M | O |
| ATOM | 6091 | OE2 | GLU | M | 161 | 52.919 | 19.846 | 26.697 | 1.00 | 37.95 | M | O |
| ATOM | 6092 | C | GLU | M | 161 | 49.062 | 24.197 | 25.339 | 1.00 | 26.71 | M | C |
| ATOM | 6093 | O | GLU | M | 161 | 48.118 | 24.600 | 26.017 | 1.00 | 32.31 | M | O |
| ATOM | 6094 | N | SER | M | 162 | 48.960 | 23.879 | 24.057 | 1.00 | 27.02 | M | N |
| ATOM | 6095 | CA | SER | M | 162 | 47.728 | 24.050 | 23.307 | 1.00 | 24.68 | M | C |
| ATOM | 6096 | CB | SER | M | 162 | 47.713 | 25.412 | 22.628 | 1.00 | 25.54 | M | C |
| ATOM | 6097 | OG | SER | M | 162 | 46.438 | 25.710 | 22.090 | 1.00 | 27.12 | M | O |
| ATOM | 6098 | C | SER | M | 162 | 47.727 | 22.970 | 22.247 | 1.00 | 25.60 | M | C |
| ATOM | 6099 | O | SER | M | 162 | 48.744 | 22.753 | 21.583 | 1.00 | 27.05 | M | O |
| ATOM | 6100 | N | VAL | M | 163 | 46.597 | 22.293 | 22.078 | 1.00 | 26.18 | M | N |
| ATOM | 6101 | CA | VAL | M | 163 | 46.503 | 21.242 | 21.075 | 1.00 | 25.33 | M | C |
| ATOM | 6102 | CB | VAL | M | 163 | 46.324 | 19.863 | 21.712 | 1.00 | 28.46 | M | C |
| ATOM | 6103 | CG1 | VAL | M | 163 | 46.447 | 18.775 | 20.653 | 1.00 | 32.28 | M | C |
| ATOM | 6104 | CG2 | VAL | M | 163 | 47.344 | 19.648 | 22.790 | 1.00 | 38.39 | M | C |
| ATOM | 6105 | C | VAL | M | 163 | 45.322 | 21.468 | 20.163 | 1.00 | 25.48 | M | C |
| ATOM | 6106 | O | VAL | M | 163 | 44.289 | 21.985 | 20.577 | 1.00 | 30.49 | M | O |
| ATOM | 6107 | N | THR | M | 164 | 45.468 | 21.070 | 18.912 | 1.00 | 25.23 | M | N |
| ATOM | 6108 | CA | THR | M | 164 | 44.367 | 21.203 | 17.972 | 1.00 | 24.82 | M | C |
| ATOM | 6109 | CB | THR | M | 164 | 44.845 | 21.180 | 16.513 | 1.00 | 25.89 | M | C |
| ATOM | 6110 | OG1 | THR | M | 164 | 45.702 | 20.049 | 16.311 | 1.00 | 25.65 | M | O |
| ATOM | 6111 | CG2 | THR | M | 164 | 45.542 | 22.477 | 16.137 | 1.00 | 18.05 | M | C |
| ATOM | 6112 | C | THR | M | 164 | 43.450 | 19.995 | 18.139 | 1.00 | 23.69 | M | C |
| ATOM | 6113 | O | THR | M | 164 | 43.749 | 19.085 | 18.912 | 1.00 | 21.85 | M | O |
| ATOM | 6114 | N | GLU | M | 165 | 42.301 | 20.037 | 17.471 | 1.00 | 20.35 | M | N |
| ATOM | 6115 | CA | GLU | M | 165 | 41.362 | 18.906 | 17.467 | 1.00 | 27.86 | M | C |
| ATOM | 6116 | CB | GLU | M | 165 | 39.964 | 19.346 | 16.991 | 1.00 | 26.70 | M | C |
| ATOM | 6117 | CG | GLU | M | 165 | 39.197 | 20.281 | 17.910 | 1.00 | 40.00 | M | C |
| ATOM | 6118 | CD | GLU | M | 165 | 38.430 | 21.361 | 17.130 | 1.00 | 54.67 | M | C |
| ATOM | 6119 | OE1 | GLU | M | 165 | 38.980 | 21.857 | 16.111 | 1.00 | 55.62 | M | O |
| ATOM | 6120 | OE2 | GLU | M | 165 | 37.290 | 21.722 | 17.535 | 1.00 | 54.22 | M | O |
| ATOM | 6121 | C | GLU | M | 165 | 41.950 | 17.970 | 16.406 | 1.00 | 19.79 | M | C |
| ATOM | 6122 | O | GLU | M | 165 | 42.769 | 18.393 | 15.581 | 1.00 | 23.55 | M | O |
| ATOM | 6123 | N | GLN | M | 166 | 41.541 | 16.711 | 16.416 | 1.00 | 26.30 | M | N |
| ATOM | 6124 | CA | GLN | M | 166 | 42.019 | 15.749 | 15.422 | 1.00 | 23.75 | M | C |
| ATOM | 6125 | CB | GLN | M | 166 | 41.270 | 14.438 | 15.581 | 1.00 | 18.96 | M | C |
| ATOM | 6126 | CG | GLN | M | 166 | 42.118 | 13.329 | 16.066 | 1.00 | 23.93 | M | C |
| ATOM | 6127 | CD | GLN | M | 166 | 41.381 | 12.025 | 16.083 | 1.00 | 26.27 | M | C |
| ATOM | 6128 | OE1 | GLN | M | 166 | 40.217 | 11.959 | 16.478 | 1.00 | 36.43 | M | O |
| ATOM | 6129 | NE2 | GLN | M | 166 | 42.054 | 10.968 | 15.675 | 1.00 | 29.79 | M | N |
| ATOM | 6130 | C | GLN | M | 166 | 41.790 | 16.301 | 14.006 | 1.00 | 24.77 | M | C |
| ATOM | 6131 | O | GLN | M | 166 | 40.726 | 16.844 | 13.718 | 1.00 | 27.83 | M | O |
| ATOM | 6132 | N | ASP | M | 167 | 42.809 | 16.233 | 13.154 | 1.00 | 26.68 | M | N |
| ATOM | 6133 | CA | ASP | M | 167 | 42.678 | 16.741 | 11.796 | 1.00 | 29.82 | M | C |
| ATOM | 6134 | CB | ASP | M | 167 | 44.016 | 16.650 | 11.063 | 1.00 | 28.28 | M | C |
| ATOM | 6135 | CG | ASP | M | 167 | 43.947 | 17.217 | 9.654 | 1.00 | 35.64 | M | C |
| ATOM | 6136 | OD1 | ASP | M | 167 | 43.662 | 18.421 | 9.517 | 1.00 | 38.25 | M | O |
| ATOM | 6137 | OD2 | ASP | M | 167 | 44.174 | 16.466 | 8.678 | 1.00 | 37.21 | M | O |
| ATOM | 6138 | C | ASP | M | 167 | 41.590 | 15.969 | 11.034 | 1.00 | 36.21 | M | C |
| ATOM | 6139 | O | ASP | M | 167 | 41.425 | 14.762 | 11.216 | 1.00 | 38.85 | M | O |
| ATOM | 6140 | N | SER | M | 168 | 40.830 | 16.685 | 10.215 | 1.00 | 35.54 | M | N |
| ATOM | 6141 | CA | SER | M | 168 | 39.757 | 16.099 | 9.428 | 1.00 | 35.90 | M | C |
| ATOM | 6142 | CB | SER | M | 168 | 38.966 | 17.216 | 8.758 | 1.00 | 37.04 | M | C |
| ATOM | 6143 | OG | SER | M | 168 | 39.848 | 18.143 | 8.145 | 1.00 | 36.99 | M | O |
| ATOM | 6144 | C | SER | M | 168 | 40.270 | 15.124 | 8.366 | 1.00 | 38.79 | M | C |
| ATOM | 6145 | O | SER | M | 168 | 39.615 | 14.130 | 8.066 | 1.00 | 35.63 | M | O |
| ATOM | 6146 | N | LYS | M | 169 | 41.462 | 15.388 | 7.835 | 1.00 | 38.57 | M | N |
| ATOM | 6147 | CA | LYS | M | 169 | 42.036 | 14.536 | 6.803 | 1.00 | 40.84 | M | C |
| ATOM | 6148 | CB | LYS | M | 169 | 42.915 | 15.361 | 5.857 | 1.00 | 46.89 | M | C |
| ATOM | 6149 | CG | LYS | M | 169 | 42.226 | 16.587 | 5.265 | 1.00 | 53.39 | M | C |
| ATOM | 6150 | CD | LYS | M | 169 | 43.218 | 17.496 | 4.550 | 1.00 | 58.45 | M | C |
| ATOM | 6151 | CE | LYS | M | 169 | 42.541 | 18.784 | 4.082 | 1.00 | 62.50 | M | C |
| ATOM | 6152 | NZ | LYS | M | 169 | 43.477 | 19.659 | 3.317 | 1.00 | 63.74 | M | N |
| ATOM | 6153 | C | LYS | M | 169 | 42.833 | 13.345 | 7.329 | 1.00 | 40.70 | M | C |
| ATOM | 6154 | O | LYS | M | 169 | 42.476 | 12.191 | 7.074 | 1.00 | 41.68 | M | O |
| ATOM | 6155 | N | ASP | M | 170 | 43.900 | 13.624 | 8.073 | 1.00 | 34.56 | M | N |
| ATOM | 6156 | CA | ASP | M | 170 | 44.770 | 12.570 | 8.579 | 1.00 | 27.55 | M | C |
| ATOM | 6157 | CB | ASP | M | 170 | 46.244 | 13.000 | 8.457 | 1.00 | 33.13 | M | C |
| ATOM | 6158 | CG | ASP | M | 170 | 46.555 | 14.265 | 9.247 | 1.00 | 34.87 | M | C |
| ATOM | 6159 | OD1 | ASP | M | 170 | 46.298 | 14.270 | 10.463 | 1.00 | 34.29 | M | O |
| ATOM | 6160 | OD2 | ASP | M | 170 | 47.046 | 15.260 | 8.665 | 1.00 | 39.83 | M | O |
| ATOM | 6161 | C | ASP | M | 170 | 44.488 | 12.056 | 9.985 | 1.00 | 21.46 | M | C |
| ATOM | 6162 | O | ASP | M | 170 | 45.105 | 11.085 | 10.417 | 1.00 | 23.23 | M | O |
| ATOM | 6163 | N | SER | M | 171 | 43.615 | 12.733 | 10.726 | 1.00 | 19.17 | M | N |
| ATOM | 6164 | CA | SER | M | 171 | 43.269 | 12.300 | 12.089 | 1.00 | 16.65 | M | C |
| ATOM | 6165 | CB | SER | M | 171 | 42.739 | 10.848 | 12.085 | 1.00 | 16.76 | M | C |
| ATOM | 6166 | OG | SER | M | 171 | 41.594 | 10.710 | 11.268 | 1.00 | 23.50 | M | O |
| ATOM | 6167 | C | SER | M | 171 | 44.414 | 12.416 | 13.106 | 1.00 | 18.10 | M | C |
| ATOM | 6168 | O | SER | M | 171 | 44.429 | 11.711 | 14.119 | 1.00 | 24.48 | M | O |
| ATOM | 6169 | N | THR | M | 172 | 45.404 | 13.251 | 12.817 | 1.00 | 19.62 | M | N |
| ATOM | 6170 | CA | THR | M | 172 | 46.496 | 13.438 | 13.764 | 1.00 | 22.48 | M | C |
| ATOM | 6171 | CB | THR | M | 172 | 47.887 | 13.605 | 13.071 | 1.00 | 24.98 | M | C |
| ATOM | 6172 | OG1 | THR | M | 172 | 47.954 | 14.870 | 12.395 | 1.00 | 19.63 | M | O |
| ATOM | 6173 | CG2 | THR | M | 172 | 48.161 | 12.457 | 12.080 | 1.00 | 21.14 | M | C |
| ATOM | 6174 | C | THR | M | 172 | 46.207 | 14.681 | 14.610 | 1.00 | 28.34 | M | C |
| ATOM | 6175 | O | THR | M | 172 | 45.165 | 15.344 | 14.454 | 1.00 | 20.01 | M | O |
| ATOM | 6176 | N | TYR | M | 173 | 47.132 | 14.975 | 15.516 | 1.00 | 27.55 | M | N |
| ATOM | 6177 | CA | TYR | M | 173 | 47.025 | 16.136 | 16.383 | 1.00 | 26.65 | M | C |
| ATOM | 6178 | CB | TYR | M | 173 | 46.942 | 15.705 | 17.849 | 1.00 | 22.59 | M | C |
| ATOM | 6179 | CG | TYR | M | 173 | 45.716 | 14.930 | 18.222 | 1.00 | 27.08 | M | C |
| ATOM | 6180 | CD1 | TYR | M | 173 | 44.561 | 15.584 | 18.682 | 1.00 | 21.00 | M | C |
| ATOM | 6181 | CE1 | TYR | M | 173 | 43.430 | 14.866 | 19.056 | 1.00 | 20.88 | M | C |
| ATOM | 6182 | CD2 | TYR | M | 173 | 45.706 | 13.532 | 18.142 | 1.00 | 26.99 | M | C |
| ATOM | 6183 | CE2 | TYR | M | 173 | 44.578 | 12.802 | 18.505 | 1.00 | 28.37 | M | C |
| ATOM | 6184 | CZ | TYR | M | 173 | 43.442 | 13.472 | 18.961 | 1.00 | 28.43 | M | C |
| ATOM | 6185 | OH | TYR | M | 173 | 42.321 | 12.743 | 19.301 | 1.00 | 29.49 | M | O |
| ATOM | 6186 | C | TYR | M | 173 | 48.288 | 16.966 | 16.226 | 1.00 | 31.63 | M | C |
| ATOM | 6187 | O | TYR | M | 173 | 49.307 | 16.473 | 15.731 | 1.00 | 37.93 | M | O |
| ATOM | 6188 | N | SER | M | 174 | 48.208 | 18.227 | 16.645 | 1.00 | 28.00 | M | N |
| ATOM | 6189 | CA | SER | M | 174 | 49.355 | 19.127 | 16.647 | 1.00 | 26.98 | M | C |
| ATOM | 6190 | CB | SER | M | 174 | 49.338 | 20.105 | 15.481 | 1.00 | 24.43 | M | C |
| ATOM | 6191 | OG | SER | M | 174 | 49.580 | 19.422 | 14.265 | 1.00 | 35.24 | M | O |
| ATOM | 6192 | C | SER | M | 174 | 49.327 | 19.837 | 17.989 | 1.00 | 26.52 | M | C |
| ATOM | 6193 | O | SER | M | 174 | 48.263 | 20.063 | 18.560 | 1.00 | 27.75 | M | O |
| ATOM | 6194 | N | LEU | M | 175 | 50.503 | 20.154 | 18.509 | 1.00 | 25.48 | M | N |
| ATOM | 6195 | CA | LEU | M | 175 | 50.599 | 20.782 | 19.810 | 1.00 | 23.45 | M | C |
| ATOM | 6196 | CB | LEU | M | 175 | 50.864 | 19.695 | 20.870 | 1.00 | 17.61 | M | C |
| ATOM | 6197 | CG | LEU | M | 175 | 50.996 | 20.003 | 22.370 | 1.00 | 27.83 | M | C |
| ATOM | 6198 | CD1 | LEU | M | 175 | 50.704 | 18.759 | 23.214 | 1.00 | 19.90 | M | C |
| ATOM | 6199 | CD2 | LEU | M | 175 | 52.372 | 20.543 | 22.688 | 1.00 | 22.94 | M | C |
| ATOM | 6200 | C | LEU | M | 175 | 51.700 | 21.829 | 19.811 | 1.00 | 28.84 | M | C |
| ATOM | 6201 | O | LEU | M | 175 | 52.671 | 21.744 | 19.053 | 1.00 | 28.22 | M | O |
| ATOM | 6202 | N | SER | M | 176 | 51.517 | 22.846 | 20.643 | 1.00 | 31.00 | M | N |
| ATOM | 6203 | CA | SER | M | 176 | 52.504 | 23.901 | 20.774 | 1.00 | 28.14 | M | C |
| ATOM | 6204 | CB | SER | M | 176 | 52.080 | 25.177 | 20.042 | 1.00 | 26.67 | M | C |
| ATOM | 6205 | OG | SER | M | 176 | 51.080 | 25.865 | 20.752 | 1.00 | 34.59 | M | O |
| ATOM | 6206 | C | SER | M | 176 | 52.705 | 24.187 | 22.243 | 1.00 | 27.29 | M | C |
| ATOM | 6207 | O | SER | M | 176 | 51.739 | 24.275 | 23.013 | 1.00 | 29.03 | M | O |
| ATOM | 6208 | N | SER | M | 177 | 53.974 | 24.215 | 22.637 | 1.00 | 23.06 | M | N |
| ATOM | 6209 | CA | SER | M | 177 | 54.355 | 24.517 | 24.002 | 1.00 | 17.26 | M | C |
| ATOM | 6210 | CB | SER | M | 177 | 55.296 | 23.469 | 24.553 | 1.00 | 21.24 | M | C |
| ATOM | 6211 | OG | SER | M | 177 | 55.344 | 23.578 | 25.958 | 1.00 | 24.89 | M | O |
| ATOM | 6212 | C | SER | M | 177 | 55.078 | 25.842 | 23.944 | 1.00 | 21.18 | M | C |
| ATOM | 6213 | O | SER | M | 177 | 56.019 | 26.010 | 23.171 | 1.00 | 26.83 | M | O |
| ATOM | 6214 | N | THR | M | 178 | 54.608 | 26.796 | 24.737 | 1.00 | 25.07 | M | N |
| ATOM | 6215 | CA | THR | M | 178 | 55.205 | 28.118 | 24.780 | 1.00 | 24.16 | M | C |
| ATOM | 6216 | CB | THR | M | 178 | 54.173 | 29.221 | 24.413 | 1.00 | 24.30 | M | C |
| ATOM | 6217 | OG1 | THR | M | 178 | 53.726 | 29.027 | 23.062 | 1.00 | 35.61 | M | O |
| ATOM | 6218 | CG2 | THR | M | 178 | 54.786 | 30.615 | 24.546 | 1.00 | 27.00 | M | C |
| ATOM | 6219 | C | THR | M | 178 | 55.813 | 28.392 | 26.148 | 1.00 | 19.60 | M | C |
| ATOM | 6220 | O | THR | M | 178 | 55.128 | 28.360 | 27.166 | 1.00 | 27.04 | M | O |
| ATOM | 6221 | N | LEU | M | 179 | 57.128 | 28.560 | 26.161 | 1.00 | 19.31 | M | N |
| ATOM | 6222 | CA | LEU | M | 179 | 57.865 | 28.876 | 27.378 | 1.00 | 19.70 | M | C |
| ATOM | 6223 | CB | LEU | M | 179 | 59.291 | 28.329 | 27.273 | 1.00 | 23.80 | M | C |
| ATOM | 6224 | CG | LEU | M | 179 | 60.243 | 28.636 | 28.427 | 1.00 | 21.55 | M | C |
| ATOM | 6225 | CD1 | LEU | M | 179 | 59.852 | 27.785 | 29.610 | 1.00 | 28.85 | M | C |
| ATOM | 6226 | CD2 | LEU | M | 179 | 61.662 | 28.312 | 27.998 | 1.00 | 27.29 | M | C |
| ATOM | 6227 | C | LEU | M | 179 | 57.903 | 30.393 | 27.379 | 1.00 | 10.60 | M | C |
| ATOM | 6228 | O | LEU | M | 179 | 58.250 | 30.996 | 26.371 | 1.00 | 15.02 | M | O |
| ATOM | 6229 | N | THR | M | 180 | 57.506 | 31.020 | 28.473 | 1.00 | 18.00 | M | N |
| ATOM | 6230 | CA | THR | M | 180 | 57.519 | 32.484 | 28.529 | 1.00 | 24.82 | M | C |
| ATOM | 6231 | CB | THR | M | 180 | 56.091 | 33.084 | 28.736 | 1.00 | 24.58 | M | C |
| ATOM | 6232 | OG1 | THR | M | 180 | 55.272 | 32.755 | 27.605 | 1.00 | 34.41 | M | O |
| ATOM | 6233 | CG2 | THR | M | 180 | 56.154 | 34.635 | 28.871 | 1.00 | 20.88 | M | C |
| ATOM | 6234 | C | THR | M | 180 | 58.447 | 32.999 | 29.622 | 1.00 | 23.08 | M | C |
| ATOM | 6235 | O | THR | M | 180 | 58.308 | 32.627 | 30.788 | 1.00 | 31.23 | M | O |
| ATOM | 6236 | N | LEU | M | 181 | 59.365 | 33.885 | 29.240 | 1.00 | 22.85 | M | N |
| ATOM | 6237 | CA | LEU | M | 181 | 60.325 | 34.468 | 30.177 | 1.00 | 22.58 | M | C |
| ATOM | 6238 | CB | LEU | M | 181 | 61.690 | 33.817 | 29.991 | 1.00 | 21.77 | M | C |
| ATOM | 6239 | CG | LEU | M | 181 | 61.705 | 32.305 | 30.160 | 1.00 | 29.34 | M | C |
| ATOM | 6240 | CD1 | LEU | M | 181 | 62.897 | 31.741 | 29.435 | 1.00 | 25.99 | M | C |
| ATOM | 6241 | CD2 | LEU | M | 181 | 61.676 | 31.916 | 31.636 | 1.00 | 27.81 | M | C |
| ATOM | 6242 | C | LEU | M | 181 | 60.502 | 35.942 | 29.926 | 1.00 | 19.42 | M | C |
| ATOM | 6243 | O | LEU | M | 181 | 60.274 | 36.422 | 28.809 | 1.00 | 22.13 | M | O |
| ATOM | 6244 | N | SER | M | 182 | 60.947 | 36.657 | 30.958 | 1.00 | 21.25 | M | N |
| ATOM | 6245 | CA | SER | M | 182 | 61.240 | 38.090 | 30.837 | 1.00 | 17.25 | M | C |
| ATOM | 6246 | CB | SER | M | 182 | 61.451 | 38.698 | 32.211 | 1.00 | 10.82 | M | C |
| ATOM | 6247 | OG | SER | M | 182 | 62.553 | 38.080 | 32.856 | 1.00 | 21.32 | M | O |
| ATOM | 6248 | C | SER | M | 182 | 62.568 | 38.128 | 30.080 | 1.00 | 22.89 | M | C |
| ATOM | 6249 | O | SER | M | 182 | 63.274 | 37.115 | 30.051 | 1.00 | 27.25 | M | O |
| ATOM | 6250 | N | LYS | M | 183 | 62.902 | 39.259 | 29.452 | 1.00 | 26.43 | M | N |
| ATOM | 6251 | CA | LYS | M | 183 | 64.173 | 39.368 | 28.730 | 1.00 | 29.27 | M | C |
| ATOM | 6252 | CB | LYS | M | 183 | 64.321 | 40.738 | 28.049 | 1.00 | 32.17 | M | C |
| ATOM | 6253 | CG | LYS | M | 183 | 65.703 | 40.977 | 27.404 | 1.00 | 26.42 | M | C |
| ATOM | 6254 | CD | LYS | M | 183 | 65.606 | 41.948 | 26.237 | 1.00 | 27.97 | M | C |
| ATOM | 6255 | CE | LYS | M | 183 | 66.531 | 43.146 | 26.400 | 1.00 | 41.37 | M | C |
| ATOM | 6256 | NZ | LYS | M | 183 | 67.980 | 42.754 | 26.561 | 1.00 | 41.48 | M | N |
| ATOM | 6257 | C | LYS | M | 183 | 65.345 | 39.136 | 29.686 | 1.00 | 28.05 | M | C |
| ATOM | 6258 | O | LYS | M | 183 | 66.309 | 38.444 | 29.352 | 1.00 | 29.29 | M | O |
| ATOM | 6259 | N | ALA | M | 184 | 65.233 | 39.681 | 30.890 | 1.00 | 27.78 | M | N |
| ATOM | 6260 | CA | ALA | M | 184 | 66.291 | 39.529 | 31.877 | 1.00 | 28.91 | M | C |
| ATOM | 6261 | CB | ALA | M | 184 | 65.946 | 40.276 | 33.163 | 1.00 | 23.17 | M | C |
| ATOM | 6262 | C | ALA | M | 184 | 66.583 | 38.058 | 32.157 | 1.00 | 25.32 | M | C |
| ATOM | 6263 | O | ALA | M | 184 | 67.724 | 37.620 | 32.045 | 1.00 | 25.71 | M | O |
| ATOM | 6264 | N | ASP | M | 185 | 65.541 | 37.298 | 32.474 | 1.00 | 27.85 | M | N |
| ATOM | 6265 | CA | ASP | M | 185 | 65.688 | 35.876 | 32.767 | 1.00 | 29.51 | M | C |
| ATOM | 6266 | CB | ASP | M | 185 | 64.376 | 35.305 | 33.306 | 1.00 | 36.62 | M | C |
| ATOM | 6267 | CG | ASP | M | 185 | 64.179 | 35.587 | 34.793 | 1.00 | 43.11 | M | C |
| ATOM | 6268 | OD1 | ASP | M | 185 | 65.097 | 36.141 | 35.442 | 1.00 | 48.61 | M | O |
| ATOM | 6269 | OD2 | ASP | M | 185 | 63.104 | 35.241 | 35.325 | 1.00 | 47.93 | M | O |
| ATOM | 6270 | C | ASP | M | 185 | 66.171 | 35.084 | 31.560 | 1.00 | 27.42 | M | C |
| ATOM | 6271 | O | ASP | M | 185 | 67.016 | 34.203 | 31.681 | 1.00 | 27.35 | M | O |
| ATOM | 6272 | N | TYR | M | 186 | 65.648 | 35.424 | 30.392 | 1.00 | 28.42 | M | N |
| ATOM | 6273 | CA | TYR | M | 186 | 66.034 | 34.761 | 29.161 | 1.00 | 29.18 | M | C |
| ATOM | 6274 | CB | TYR | M | 186 | 65.205 | 35.311 | 28.002 | 1.00 | 21.24 | M | C |
| ATOM | 6275 | CG | TYR | M | 186 | 65.579 | 34.771 | 26.645 | 1.00 | 22.51 | M | C |
| ATOM | 6276 | CD1 | TYR | M | 186 | 65.456 | 33.414 | 26.346 | 1.00 | 21.80 | M | C |
| ATOM | 6277 | CE1 | TYR | M | 186 | 65.803 | 32.917 | 25.072 | 1.00 | 21.42 | M | C |
| ATOM | 6278 | CD2 | TYR | M | 186 | 66.052 | 35.625 | 25.648 | 1.00 | 24.11 | M | C |
| ATOM | 6279 | CE2 | TYR | M | 186 | 66.392 | 35.145 | 24.381 | 1.00 | 23.53 | M | C |
| ATOM | 6280 | CZ | TYR | M | 186 | 66.265 | 33.791 | 24.103 | 1.00 | 21.92 | M | C |
| ATOM | 6281 | OH | TYR | M | 186 | 66.602 | 33.335 | 22.851 | 1.00 | 24.77 | M | O |
| ATOM | 6282 | C | TYR | M | 186 | 67.534 | 34.967 | 28.894 | 1.00 | 34.31 | M | C |
| ATOM | 6283 | O | TYR | M | 186 | 68.215 | 34.049 | 28.435 | 1.00 | 34.35 | M | O |
| ATOM | 6284 | N | GLU | M | 187 | 68.062 | 36.142 | 29.235 | 1.00 | 35.63 | M | N |
| ATOM | 6285 | CA | GLU | M | 187 | 69.478 | 36.409 | 28.986 | 1.00 | 39.08 | M | C |
| ATOM | 6286 | CB | GLU | M | 187 | 69.732 | 37.904 | 28.808 | 1.00 | 36.88 | M | C |
| ATOM | 6287 | CG | GLU | M | 187 | 69.333 | 38.410 | 27.422 | 1.00 | 35.56 | M | C |
| ATOM | 6288 | CD | GLU | M | 187 | 69.270 | 39.922 | 27.334 | 1.00 | 34.45 | M | C |
| ATOM | 6289 | OE1 | GLU | M | 187 | 69.264 | 40.583 | 28.388 | 1.00 | 36.02 | M | O |
| ATOM | 6290 | OE2 | GLU | M | 187 | 69.224 | 40.453 | 26.204 | 1.00 | 37.21 | M | O |
| ATOM | 6291 | C | GLU | M | 187 | 70.476 | 35.792 | 29.959 | 1.00 | 39.45 | M | C |
| ATOM | 6292 | O | GLU | M | 187 | 71.694 | 35.862 | 29.733 | 1.00 | 42.36 | M | O |
| ATOM | 6293 | N | LYS | M | 188 | 69.977 | 35.133 | 31.001 | 1.00 | 33.62 | M | N |
| ATOM | 6294 | CA | LYS | M | 188 | 70.888 | 34.519 | 31.944 | 1.00 | 34.96 | M | C |
| ATOM | 6295 | CB | LYS | M | 188 | 70.448 | 34.752 | 33.393 | 1.00 | 32.74 | M | C |
| ATOM | 6296 | CG | LYS | M | 188 | 69.163 | 34.133 | 33.809 | 1.00 | 36.81 | M | C |
| ATOM | 6297 | CD | LYS | M | 188 | 68.813 | 34.554 | 35.219 | 1.00 | 34.78 | M | C |
| ATOM | 6298 | CE | LYS | M | 188 | 68.836 | 36.064 | 35.357 | 1.00 | 34.58 | M | C |
| ATOM | 6299 | NZ | LYS | M | 188 | 68.233 | 36.495 | 36.646 | 1.00 | 39.61 | M | N |
| ATOM | 6300 | C | LYS | M | 188 | 71.256 | 33.059 | 31.668 | 1.00 | 35.01 | M | C |
| ATOM | 6301 | O | LYS | M | 188 | 71.933 | 32.427 | 32.474 | 1.00 | 39.59 | M | O |
| ATOM | 6302 | N | HIS | M | 189 | 70.861 | 32.551 | 30.502 | 1.00 | 35.43 | M | N |
| ATOM | 6303 | CA | HIS | M | 189 | 71.161 | 31.177 | 30.089 | 1.00 | 32.81 | M | C |
| ATOM | 6304 | CB | HIS | M | 189 | 70.035 | 30.236 | 30.474 | 1.00 | 33.07 | M | C |
| ATOM | 6305 | CG | HIS | M | 189 | 69.732 | 30.244 | 31.933 | 1.00 | 37.06 | M | C |
| ATOM | 6306 | CD2 | HIS | M | 189 | 68.696 | 30.775 | 32.625 | 1.00 | 36.31 | M | C |
| ATOM | 6307 | ND1 | HIS | M | 189 | 70.596 | 29.720 | 32.871 | 1.00 | 42.03 | M | N |
| ATOM | 6308 | CE1 | HIS | M | 189 | 70.106 | 29.933 | 34.081 | 1.00 | 45.14 | M | C |
| ATOM | 6309 | NE2 | HIS | M | 189 | 68.954 | 30.572 | 33.959 | 1.00 | 41.83 | M | N |
| ATOM | 6310 | C | HIS | M | 189 | 71.323 | 31.185 | 28.588 | 1.00 | 34.32 | M | C |
| ATOM | 6311 | O | HIS | M | 189 | 70.807 | 32.085 | 27.919 | 1.00 | 33.56 | M | O |
| ATOM | 6312 | N | LYS | M | 190 | 72.037 | 30.205 | 28.041 | 1.00 | 37.56 | M | N |
| ATOM | 6313 | CA | LYS | M | 190 | 72.227 | 30.203 | 26.594 | 1.00 | 43.16 | M | C |
| ATOM | 6314 | CB | LYS | M | 190 | 73.686 | 30.542 | 26.206 | 1.00 | 47.78 | M | C |
| ATOM | 6315 | CG | LYS | M | 190 | 74.770 | 29.543 | 26.614 | 1.00 | 56.89 | M | C |
| ATOM | 6316 | CD | LYS | M | 190 | 75.298 | 28.745 | 25.395 | 1.00 | 63.86 | M | C |
| ATOM | 6317 | CE | LYS | M | 190 | 75.826 | 29.651 | 24.269 | 1.00 | 68.61 | M | C |
| ATOM | 6318 | NZ | LYS | M | 190 | 76.205 | 28.889 | 23.036 | 1.00 | 64.89 | M | N |
| ATOM | 6319 | C | LYS | M | 190 | 71.659 | 29.041 | 25.785 | 1.00 | 37.25 | M | C |
| ATOM | 6320 | O | LYS | M | 190 | 71.265 | 29.238 | 24.638 | 1.00 | 38.53 | M | O |
| ATOM | 6321 | N | VAL | M | 191 | 71.612 | 27.843 | 26.354 | 1.00 | 36.98 | M | N |
| ATOM | 6322 | CA | VAL | M | 191 | 71.052 | 26.699 | 25.622 | 1.00 | 39.04 | M | C |
| ATOM | 6323 | CB | VAL | M | 191 | 71.797 | 25.357 | 25.918 | 1.00 | 44.31 | M | C |
| ATOM | 6324 | CG1 | VAL | M | 191 | 71.258 | 24.262 | 25.005 | 1.00 | 41.79 | M | C |
| ATOM | 6325 | CG2 | VAL | M | 191 | 73.313 | 25.508 | 25.712 | 1.00 | 42.17 | M | C |
| ATOM | 6326 | C | VAL | M | 191 | 69.567 | 26.516 | 25.960 | 1.00 | 39.92 | M | C |
| ATOM | 6327 | O | VAL | M | 191 | 69.190 | 26.327 | 27.135 | 1.00 | 33.27 | M | O |
| ATOM | 6328 | N | TYR | M | 192 | 68.735 | 26.597 | 24.926 | 1.00 | 30.46 | M | N |
| ATOM | 6329 | CA | TYR | M | 192 | 67.294 | 26.432 | 25.072 | 1.00 | 30.07 | M | C |
| ATOM | 6330 | CB | TYR | M | 192 | 66.580 | 27.677 | 24.566 | 1.00 | 28.49 | M | C |
| ATOM | 6331 | CG | TYR | M | 192 | 66.652 | 28.808 | 25.548 | 1.00 | 30.17 | M | C |
| ATOM | 6332 | CD1 | TYR | M | 192 | 67.758 | 29.661 | 25.596 | 1.00 | 34.87 | M | C |
| ATOM | 6333 | CE1 | TYR | M | 192 | 67.850 | 30.669 | 26.582 | 1.00 | 31.10 | M | C |
| ATOM | 6334 | CD2 | TYR | M | 192 | 65.645 | 28.985 | 26.490 | 1.00 | 31.99 | M | C |
| ATOM | 6335 | CE2 | TYR | M | 192 | 65.725 | 29.971 | 27.462 | 1.00 | 31.06 | M | C |
| ATOM | 6336 | CZ | TYR | M | 192 | 66.827 | 30.807 | 27.504 | 1.00 | 31.05 | M | C |
| ATOM | 6337 | OH | TYR | M | 192 | 66.873 | 31.775 | 28.469 | 1.00 | 31.69 | M | O |
| ATOM | 6338 | C | TYR | M | 192 | 66.813 | 25.195 | 24.324 | 1.00 | 31.40 | M | C |
| ATOM | 6339 | O | TYR | M | 192 | 67.016 | 25.084 | 23.119 | 1.00 | 29.12 | M | O |
| ATOM | 6340 | N | ALA | M | 193 | 66.152 | 24.283 | 25.033 | 1.00 | 31.81 | M | N |
| ATOM | 6341 | CA | ALA | M | 193 | 65.680 | 23.055 | 24.405 | 1.00 | 35.43 | M | C |
| ATOM | 6342 | CB | ALA | M | 193 | 66.763 | 21.985 | 24.551 | 1.00 | 38.83 | M | C |
| ATOM | 6343 | C | ALA | M | 193 | 64.330 | 22.465 | 24.841 | 1.00 | 37.79 | M | C |
| ATOM | 6344 | O | ALA | M | 193 | 63.916 | 22.590 | 26.000 | 1.00 | 29.57 | M | O |
| ATOM | 6345 | N | CYS | M | 194 | 63.638 | 21.835 | 23.891 | 1.00 | 34.97 | M | N |
| ATOM | 6346 | CA | CYS | M | 194 | 62.392 | 21.148 | 24.215 | 1.00 | 41.35 | M | C |
| ATOM | 6347 | C | CYS | M | 194 | 62.584 | 19.669 | 23.876 | 1.00 | 38.31 | M | C |
| ATOM | 6348 | O | CYS | M | 194 | 63.051 | 19.324 | 22.790 | 1.00 | 36.25 | M | O |
| ATOM | 6349 | CB | CYS | M | 194 | 61.148 | 21.758 | 23.534 | 1.00 | 37.37 | M | C |
| ATOM | 6350 | SG | CYS | M | 194 | 61.044 | 21.755 | 21.719 | 1.00 | 50.39 | M | S |
| ATOM | 6351 | N | GLU | M | 195 | 62.343 | 18.820 | 24.870 | 1.00 | 36.71 | M | N |
| ATOM | 6352 | CA | GLU | M | 195 | 62.485 | 17.379 | 24.728 | 1.00 | 36.00 | M | C |
| ATOM | 6353 | CB | GLU | M | 195 | 63.277 | 16.800 | 25.909 | 1.00 | 32.09 | M | C |
| ATOM | 6354 | CG | GLU | M | 195 | 63.765 | 15.386 | 25.653 | 1.00 | 42.90 | M | C |
| ATOM | 6355 | CD | GLU | M | 195 | 64.772 | 14.880 | 26.669 | 1.00 | 45.58 | M | C |
| ATOM | 6356 | OE1 | GLU | M | 195 | 65.334 | 15.695 | 27.431 | 1.00 | 50.94 | M | O |
| ATOM | 6357 | OE2 | GLU | M | 195 | 65.017 | 13.656 | 26.690 | 1.00 | 47.57 | M | O |
| ATOM | 6358 | C | GLU | M | 195 | 61.105 | 16.731 | 24.633 | 1.00 | 34.02 | M | C |
| ATOM | 6359 | O | GLU | M | 195 | 60.321 | 16.755 | 25.592 | 1.00 | 31.64 | M | O |
| ATOM | 6360 | N | VAL | M | 196 | 60.839 | 16.127 | 23.478 | 1.00 | 32.39 | M | N |
| ATOM | 6361 | CA | VAL | M | 196 | 59.561 | 15.473 | 23.197 | 1.00 | 32.99 | M | C |
| ATOM | 6362 | CB | VAL | M | 196 | 59.092 | 15.809 | 21.739 | 1.00 | 25.93 | M | C |
| ATOM | 6363 | CG1 | VAL | M | 196 | 57.817 | 15.066 | 21.391 | 1.00 | 17.80 | M | C |
| ATOM | 6364 | CG2 | VAL | M | 196 | 58.888 | 17.313 | 21.585 | 1.00 | 19.74 | M | C |
| ATOM | 6365 | C | VAL | M | 196 | 59.595 | 13.947 | 23.374 | 1.00 | 34.80 | M | C |
| ATOM | 6366 | O | VAL | M | 196 | 60.428 | 13.258 | 22.778 | 1.00 | 34.72 | M | O |
| ATOM | 6367 | N | THR | M | 197 | 58.681 | 13.438 | 24.196 | 1.00 | 34.31 | M | N |
| ATOM | 6368 | CA | THR | M | 197 | 58.528 | 12.003 | 24.447 | 1.00 | 36.85 | M | C |
| ATOM | 6369 | CB | THR | M | 197 | 58.550 | 11.709 | 25.960 | 1.00 | 40.18 | M | C |
| ATOM | 6370 | OG1 | THR | M | 197 | 59.771 | 12.200 | 26.529 | 1.00 | 43.13 | M | O |
| ATOM | 6371 | CG2 | THR | M | 197 | 58.440 | 10.215 | 26.213 | 1.00 | 42.27 | M | C |
| ATOM | 6372 | C | THR | M | 197 | 57.158 | 11.565 | 23.874 | 1.00 | 39.92 | M | C |
| ATOM | 6373 | O | THR | M | 197 | 56.105 | 12.094 | 24.269 | 1.00 | 38.19 | M | O |
| ATOM | 6374 | N | HIS | M | 198 | 57.168 | 10.599 | 22.958 | 1.00 | 40.05 | M | N |
| ATOM | 6375 | CA | HIS | M | 198 | 55.933 | 10.112 | 22.320 | 1.00 | 37.71 | M | C |
| ATOM | 6376 | CB | HIS | M | 198 | 55.607 | 11.016 | 21.123 | 1.00 | 32.34 | M | C |
| ATOM | 6377 | CG | HIS | M | 198 | 54.356 | 10.644 | 20.393 | 1.00 | 35.35 | M | C |
| ATOM | 6378 | CD2 | HIS | M | 198 | 53.094 | 11.125 | 20.489 | 1.00 | 35.84 | M | C |
| ATOM | 6379 | ND1 | HIS | M | 198 | 54.334 | 9.710 | 19.377 | 1.00 | 32.65 | M | N |
| ATOM | 6380 | CE1 | HIS | M | 198 | 53.115 | 9.640 | 18.874 | 1.00 | 33.13 | M | C |
| ATOM | 6381 | NE2 | HIS | M | 198 | 52.343 | 10.489 | 19.531 | 1.00 | 34.54 | M | N |
| ATOM | 6382 | C | HIS | M | 198 | 56.137 | 8.654 | 21.871 | 1.00 | 38.87 | M | C |
| ATOM | 6383 | O | HIS | M | 198 | 57.249 | 8.289 | 21.458 | 1.00 | 38.21 | M | O |
| ATOM | 6384 | N | GLN | M | 199 | 55.077 | 7.836 | 21.902 | 1.00 | 33.59 | M | N |
| ATOM | 6385 | CA | GLN | M | 199 | 55.232 | 6.429 | 21.524 | 1.00 | 31.69 | M | C |
| ATOM | 6386 | CB | GLN | M | 199 | 53.985 | 5.587 | 21.844 | 1.00 | 32.46 | M | C |
| ATOM | 6387 | CG | GLN | M | 199 | 52.951 | 5.412 | 20.733 | 1.00 | 34.75 | M | C |
| ATOM | 6388 | CD | GLN | M | 199 | 51.921 | 4.320 | 21.062 | 1.00 | 42.85 | M | C |
| ATOM | 6389 | OE1 | GLN | M | 199 | 51.762 | 3.345 | 20.316 | 1.00 | 42.07 | M | O |
| ATOM | 6390 | NE2 | GLN | M | 199 | 51.236 | 4.471 | 22.192 | 1.00 | 40.44 | M | N |
| ATOM | 6391 | C | GLN | M | 199 | 55.749 | 6.175 | 20.103 | 1.00 | 38.62 | M | C |
| ATOM | 6392 | O | GLN | M | 199 | 56.354 | 5.133 | 19.845 | 1.00 | 38.02 | M | O |
| ATOM | 6393 | N | GLY | M | 200 | 55.559 | 7.141 | 19.203 | 1.00 | 38.12 | M | N |
| ATOM | 6394 | CA | GLY | M | 200 | 56.039 | 6.997 | 17.839 | 1.00 | 38.26 | M | C |
| ATOM | 6395 | C | GLY | M | 200 | 57.542 | 7.198 | 17.659 | 1.00 | 40.70 | M | C |
| ATOM | 6396 | O | GLY | M | 200 | 58.068 | 6.966 | 16.571 | 1.00 | 42.21 | M | O |
| ATOM | 6397 | N | LEU | M | 201 | 58.217 | 7.727 | 18.680 | 1.00 | 41.04 | M | N |
| ATOM | 6399 | CA | LEU | M | 201 | 59.665 | 7.891 | 18.644 | 1.00 | 40.77 | M | C |
| ATOM | 6400 | CB | LEU | M | 201 | 60.055 | 9.269 | 19.184 | 1.00 | 42.42 | M | C |
| ATOM | 6401 | CG | LEU | M | 201 | 59.259 | 10.460 | 18.645 | 1.00 | 45.68 | M | C |
| ATOM | 6402 | CD1 | LEU | M | 201 | 59.187 | 11.556 | 19.697 | 1.00 | 46.60 | M | C |
| ATOM | 6403 | CD2 | LEU | M | 201 | 59.904 | 10.980 | 17.371 | 1.00 | 45.35 | M | C |
| ATOM | 6404 | C | LEU | M | 201 | 60.359 | 6.803 | 19.455 | 1.00 | 43.25 | M | C |
| ATOM | 6405 | O | LEU | M | 201 | 59.865 | 6.450 | 20.549 | 1.00 | 40.27 | M | O |
| ATOM | 6406 | N | SER | M | 202 | 61.428 | 6.233 | 18.924 | 1.00 | 44.06 | M | N |
| ATOM | 6407 | CA | SER | M | 202 | 62.156 | 5.164 | 19.627 | 1.00 | 40.91 | M | C |
| ATOM | 6408 | CB | SER | M | 202 | 63.184 | 4.518 | 18.700 | 1.00 | 41.50 | M | C |
| ATOM | 6409 | OG | SER | M | 202 | 64.002 | 5.498 | 18.074 | 1.00 | 47.74 | M | O |
| ATOM | 6410 | C | SER | M | 202 | 62.822 | 5.682 | 20.912 | 1.00 | 41.13 | M | C |
| ATOM | 6411 | O | SER | M | 202 | 63.004 | 4.935 | 21.878 | 1.00 | 43.56 | M | O |
| ATOM | 6412 | N | SER | M | 203 | 63.161 | 6.969 | 20.915 | 1.00 | 32.15 | M | N |
| ATOM | 6413 | CA | SER | M | 203 | 63.769 | 7.628 | 22.068 | 1.00 | 33.93 | M | C |
| ATOM | 6414 | CB | SER | M | 203 | 65.286 | 7.428 | 22.065 | 1.00 | 31.25 | M | C |
| ATOM | 6415 | OG | SER | M | 203 | 65.837 | 7.888 | 20.846 | 1.00 | 32.53 | M | O |
| ATOM | 6416 | C | SER | M | 203 | 63.423 | 9.121 | 21.989 | 1.00 | 34.67 | M | C |
| ATOM | 6417 | O | SER | M | 203 | 63.160 | 9.648 | 20.902 | 1.00 | 36.83 | M | O |
| ATOM | 6418 | N | PRO | M | 204 | 63.367 | 9.812 | 23.143 | 1.00 | 32.23 | M | N |
| ATOM | 6419 | CD | PRO | M | 204 | 63.325 | 9.250 | 24.504 | 1.00 | 28.99 | M | C |
| ATOM | 6420 | CA | PRO | M | 204 | 63.042 | 11.242 | 23.172 | 1.00 | 31.90 | M | C |
| ATOM | 6421 | CB | PRO | M | 204 | 63.299 | 11.602 | 24.619 | 1.00 | 31.50 | M | C |
| ATOM | 6422 | CG | PRO | M | 204 | 62.740 | 10.400 | 25.309 | 1.00 | 31.62 | M | C |
| ATOM | 6423 | C | PRO | M | 204 | 63.807 | 12.132 | 22.196 | 1.00 | 37.02 | M | C |
| ATOM | 6424 | O | PRO | M | 204 | 65.011 | 11.980 | 22.012 | 1.00 | 40.80 | M | O |
| ATOM | 6425 | N | VAL | M | 205 | 63.077 | 13.022 | 21.528 | 1.00 | 36.19 | M | N |
| ATOM | 6426 | CA | VAL | M | 205 | 63.672 | 13.939 | 20.569 | 1.00 | 33.25 | M | C |
| ATOM | 6427 | CB | VAL | M | 205 | 62.824 | 14.040 | 19.296 | 1.00 | 32.35 | M | C |
| ATOM | 6428 | CG1 | VAL | M | 205 | 63.256 | 15.249 | 18.448 | 1.00 | 32.96 | M | C |
| ATOM | 6429 | CG2 | VAL | M | 205 | 62.983 | 12.769 | 18.497 | 1.00 | 37.30 | M | C |
| ATOM | 6430 | C | VAL | M | 205 | 63.856 | 15.321 | 21.179 | 1.00 | 36.33 | M | C |
| ATOM | 6431 | O | VAL | M | 205 | 62.952 | 15.852 | 21.830 | 1.00 | 33.96 | M | O |
| ATOM | 6432 | N | THR | M | 206 | 65.022 | 15.910 | 20.932 | 1.00 | 37.43 | M | N |
| ATOM | 6433 | CA | THR | M | 206 | 65.349 | 17.224 | 21.465 | 1.00 | 34.73 | M | C |
| ATOM | 6434 | CB | THR | M | 206 | 66.538 | 17.149 | 22.462 | 1.00 | 34.68 | M | C |
| ATOM | 6435 | OG1 | THR | M | 206 | 66.111 | 16.518 | 23.678 | 1.00 | 37.99 | M | O |
| ATOM | 6436 | CG2 | THR | M | 206 | 67.076 | 18.537 | 22.784 | 1.00 | 39.06 | M | C |
| ATOM | 6437 | C | THR | M | 206 | 65.702 | 18.192 | 20.369 | 1.00 | 34.51 | M | C |
| ATOM | 6438 | O | THR | M | 206 | 66.512 | 17.893 | 19.498 | 1.00 | 43.77 | M | O |
| ATOM | 6439 | N | LYS | M | 207 | 65.008 | 19.316 | 20.350 | 1.00 | 35.24 | M | N |
| ATOM | 6440 | CA | LYS | M | 207 | 65.306 | 20.357 | 19.386 | 1.00 | 33.45 | M | C |
| ATOM | 6441 | CB | LYS | M | 207 | 64.060 | 20.760 | 18.596 | 1.00 | 33.95 | M | C |
| ATOM | 6442 | CG | LYS | M | 207 | 63.706 | 19.747 | 17.491 | 1.00 | 34.37 | M | C |
| ATOM | 6443 | CD | LYS | M | 207 | 64.805 | 19.698 | 16.421 | 1.00 | 32.91 | M | C |
| ATOM | 6444 | CE | LYS | M | 207 | 64.516 | 18.742 | 15.272 | 1.00 | 31.61 | M | C |
| ATOM | 6445 | NZ | LYS | M | 207 | 64.488 | 17.311 | 15.690 | 1.00 | 38.06 | M | N |
| ATOM | 6446 | C | LYS | M | 207 | 65.864 | 21.502 | 20.226 | 1.00 | 36.35 | M | C |
| ATOM | 6447 | O | LYS | M | 207 | 65.482 | 21.674 | 21.398 | 1.00 | 29.57 | M | O |
| ATOM | 6448 | N | SER | M | 208 | 66.835 | 22.222 | 19.677 | 1.00 | 36.04 | M | N |
| ATOM | 6449 | CA | SER | M | 208 | 67.430 | 23.306 | 20.431 | 1.00 | 34.12 | M | C |
| ATOM | 6450 | CB | SER | M | 208 | 68.399 | 22.748 | 21.477 | 1.00 | 39.65 | M | C |
| ATOM | 6451 | OG | SER | M | 208 | 69.525 | 22.171 | 20.847 | 1.00 | 45.81 | M | O |
| ATOM | 6452 | C | SER | M | 208 | 68.165 | 24.323 | 19.603 | 1.00 | 34.61 | M | C |
| ATOM | 6453 | O | SER | M | 208 | 68.338 | 24.171 | 18.390 | 1.00 | 32.40 | M | O |
| ATOM | 6454 | N | PHE | M | 209 | 68.546 | 25.393 | 20.290 | 1.00 | 32.79 | M | N |
| ATOM | 6455 | CA | PHE | M | 209 | 69.310 | 26.481 | 19.710 | 1.00 | 36.84 | M | C |
| ATOM | 6456 | CB | PHE | M | 209 | 68.392 | 27.511 | 19.036 | 1.00 | 34.09 | M | C |
| ATOM | 6457 | CG | PHE | M | 209 | 67.485 | 28.243 | 19.988 | 1.00 | 38.50 | M | C |
| ATOM | 6458 | CD1 | PHE | M | 209 | 67.937 | 29.354 | 20.693 | 1.00 | 36.94 | M | C |
| ATOM | 6459 | CD2 | PHE | M | 209 | 66.172 | 27.820 | 20.185 | 1.00 | 38.33 | M | C |
| ATOM | 6460 | CE1 | PHE | M | 209 | 67.096 | 30.029 | 21.580 | 1.00 | 37.56 | M | C |
| ATOM | 6461 | CE2 | PHE | M | 209 | 65.324 | 28.494 | 21.072 | 1.00 | 35.91 | M | C |
| ATOM | 6462 | CZ | PHE | M | 209 | 65.788 | 29.595 | 21.767 | 1.00 | 34.72 | M | C |
| ATOM | 6463 | C | PHE | M | 209 | 70.089 | 27.129 | 20.846 | 1.00 | 37.85 | M | C |
| ATOM | 6464 | O | PHE | M | 209 | 69.790 | 26.892 | 22.023 | 1.00 | 36.01 | M | O |
| ATOM | 6465 | N | ASN | M | 210 | 71.136 | 27.871 | 20.489 | 1.00 | 39.26 | M | N |
| ATOM | 6466 | CA | ASN | M | 210 | 71.943 | 28.601 | 21.468 | 1.00 | 38.68 | M | C |
| ATOM | 6467 | CB | ASN | M | 210 | 73.431 | 28.458 | 21.165 | 1.00 | 40.95 | M | C |
| ATOM | 6468 | CG | ASN | M | 210 | 73.919 | 27.040 | 21.313 | 1.00 | 45.83 | M | C |
| ATOM | 6469 | OD1 | ASN | M | 210 | 73.768 | 26.417 | 22.368 | 1.00 | 50.62 | M | O |
| ATOM | 6470 | ND2 | ASN | M | 210 | 74.529 | 26.522 | 20.258 | 1.00 | 45.60 | M | N |
| ATOM | 6471 | C | ASN | M | 210 | 71.539 | 30.057 | 21.302 | 1.00 | 33.12 | M | C |
| ATOM | 6472 | O | ASN | M | 210 | 71.468 | 30.544 | 20.178 | 1.00 | 37.26 | M | O |
| ATOM | 6473 | N | ARG | M | 211 | 71.277 | 30.746 | 22.407 | 1.00 | 32.32 | M | N |
| ATOM | 6474 | CA | ARG | M | 211 | 70.853 | 32.158 | 22.378 | 1.00 | 36.30 | M | C |
| ATOM | 6475 | CB | ARG | M | 211 | 70.692 | 32.676 | 23.811 | 1.00 | 36.21 | M | C |
| ATOM | 6476 | CG | ARG | M | 211 | 70.009 | 34.036 | 23.945 | 1.00 | 32.54 | M | C |
| ATOM | 6477 | CD | ARG | M | 211 | 69.722 | 34.361 | 25.413 | 1.00 | 29.26 | M | C |
| ATOM | 6478 | NE | ARG | M | 211 | 70.935 | 34.330 | 26.223 | 1.00 | 28.98 | M | N |
| ATOM | 6479 | CZ | ARG | M | 211 | 71.919 | 35.224 | 26.130 | 1.00 | 30.88 | M | C |
| ATOM | 6480 | NH1 | ARG | M | 211 | 71.836 | 36.240 | 25.275 | 1.00 | 26.75 | M | N |
| ATOM | 6481 | NH2 | ARG | M | 211 | 73.017 | 35.065 | 26.843 | 1.00 | 27.22 | M | N |
| ATOM | 6482 | C | ARG | M | 211 | 71.793 | 33.065 | 21.576 | 1.00 | 36.33 | M | C |
| ATOM | 6483 | O | ARG | M | 211 | 72.998 | 33.091 | 21.816 | 1.00 | 42.87 | M | O |
| ATOM | 6484 | N | GLY | M | 212 | 71.231 | 33.791 | 20.613 | 1.00 | 42.91 | M | N |
| ATOM | 6485 | CA | GLY | M | 212 | 72.030 | 34.668 | 19.768 | 1.00 | 53.40 | M | C |
| ATOM | 6486 | C | GLY | M | 212 | 72.587 | 34.004 | 18.504 | 1.00 | 59.55 | M | C |
| ATOM | 6487 | O | GLY | M | 212 | 71.883 | 33.859 | 17.494 | 1.00 | 63.76 | M | O |
| ATOM | 6488 | N | GLU | M | 213 | 73.851 | 33.587 | 18.561 | 1.00 | 62.11 | M | N |
| ATOM | 6489 | CA | GLU | M | 213 | 74.513 | 32.945 | 17.422 | 1.00 | 64.30 | M | C |
| ATOM | 6490 | CB | GLU | M | 213 | 76.031 | 32.918 | 17.651 | 1.00 | 69.46 | M | C |
| ATOM | 6491 | CG | GLU | M | 213 | 76.679 | 34.317 | 17.733 | 1.00 | 72.81 | M | C |
| ATOM | 6492 | CD | GLU | M | 213 | 78.104 | 34.298 | 18.292 | 1.00 | 73.54 | M | C |
| ATOM | 6493 | OE1 | GLU | M | 213 | 79.009 | 33.724 | 17.635 | 1.00 | 73.81 | M | O |
| ATOM | 6494 | OE2 | GLU | M | 213 | 78.312 | 34.867 | 19.390 | 1.00 | 70.59 | M | O |
| ATOM | 6495 | C | GLU | M | 213 | 73.990 | 31.536 | 17.132 | 1.00 | 62.52 | M | C |
| ATOM | 6496 | O | GLU | M | 213 | 74.470 | 30.548 | 17.699 | 1.00 | 60.47 | M | O |
| ATOM | 6497 | N | GLN | W | 1 | 39.524 | 10.104 | −28.763 | 1.00 | 60.29 | W | N |
| ATOM | 6498 | CA | GLN | W | 1 | 38.919 | 11.071 | −27.849 | 1.00 | 63.42 | W | C |
| ATOM | 6499 | CB | GLN | W | 1 | 39.873 | 12.235 | −27.605 | 1.00 | 64.15 | W | C |
| ATOM | 6500 | CG | GLN | W | 1 | 41.333 | 11.837 | −27.484 | 1.00 | 70.41 | W | C |
| ATOM | 6501 | CD | GLN | W | 1 | 42.260 | 12.986 | −27.843 | 1.00 | 72.66 | W | C |
| ATOM | 6502 | OE1 | GLN | W | 1 | 41.838 | 14.148 | −27.881 | 1.00 | 74.93 | W | O |
| ATOM | 6503 | NE2 | GLN | W | 1 | 43.523 | 12.668 | −28.131 | 1.00 | 71.30 | W | N |
| ATOM | 6504 | C | GLN | W | 1 | 37.589 | 11.619 | −28.387 | 1.00 | 60.81 | W | C |
| ATOM | 6505 | O | GLN | W | 1 | 36.888 | 12.353 | −27.683 | 1.00 | 61.83 | W | O |
| ATOM | 6506 | N | ALA | W | 2 | 37.264 | 11.265 | −29.632 | 1.00 | 56.77 | W | N |
| ATOM | 6507 | CA | ALA | W | 2 | 36.026 | 11.676 | −30.318 | 1.00 | 53.91 | W | C |
| ATOM | 6508 | CB | ALA | W | 2 | 34.810 | 11.527 | −29.405 | 1.00 | 50.35 | W | C |
| ATOM | 6509 | C | ALA | W | 2 | 36.049 | 13.067 | −30.941 | 1.00 | 49.81 | W | C |
| ATOM | 6510 | O | ALA | W | 2 | 36.356 | 14.054 | −30.271 | 1.00 | 54.91 | W | O |
| ATOM | 6511 | N | VAL | W | 3 | 35.688 | 13.128 | −32.223 | 1.00 | 40.63 | W | N |
| ATOM | 6512 | CA | VAL | W | 3 | 35.660 | 14.374 | −32.997 | 1.00 | 38.80 | W | C |
| ATOM | 6513 | CB | VAL | W | 3 | 36.543 | 14.250 | −34.294 | 1.00 | 36.33 | W | C |
| ATOM | 6514 | CG1 | VAL | W | 3 | 36.614 | 15.580 | −35.026 | 1.00 | 35.97 | W | C |
| ATOM | 6515 | CG2 | VAL | W | 3 | 37.942 | 13.771 | −33.945 | 1.00 | 38.18 | W | C |
| ATOM | 6516 | C | VAL | W | 3 | 34.219 | 14.709 | −33.421 | 1.00 | 38.28 | W | C |
| ATOM | 6517 | O | VAL | W | 3 | 33.503 | 13.866 | −33.974 | 1.00 | 31.65 | W | O |
| ATOM | 6518 | N | VAL | W | 4 | 33.820 | 15.954 | −33.196 | 1.00 | 37.02 | W | N |
| ATOM | 6519 | CA | VAL | W | 4 | 32.482 | 16.433 | −33.541 | 1.00 | 29.28 | W | C |
| ATOM | 6520 | CB | VAL | W | 4 | 31.831 | 17.134 | −32.303 | 1.00 | 29.24 | W | C |
| ATOM | 6521 | CG1 | VAL | W | 4 | 30.498 | 17.775 | −32.667 | 1.00 | 23.16 | W | C |
| ATOM | 6522 | CG2 | VAL | W | 4 | 31.655 | 16.128 | −31.180 | 1.00 | 17.40 | W | C |
| ATOM | 6523 | C | VAL | W | 4 | 32.608 | 17.414 | −34.714 | 1.00 | 31.92 | W | C |
| ATOM | 6524 | O | VAL | W | 4 | 33.174 | 18.496 | −34.559 | 1.00 | 30.19 | W | O |
| ATOM | 6525 | N | THR | W | 5 | 32.082 | 17.040 | −35.883 | 1.00 | 33.59 | W | N |
| ATOM | 6526 | CA | THR | W | 5 | 32.199 | 17.900 | −37.062 | 1.00 | 31.62 | W | C |
| ATOM | 6527 | CB | THR | W | 5 | 32.765 | 17.128 | −38.282 | 1.00 | 34.41 | W | C |
| ATOM | 6528 | OG1 | THR | W | 5 | 31.837 | 16.121 | −38.678 | 1.00 | 46.83 | W | O |
| ATOM | 6529 | CG2 | THR | W | 5 | 34.088 | 16.457 | −37.949 | 1.00 | 34.96 | W | C |
| ATOM | 6530 | C | THR | W | 5 | 30.917 | 18.603 | −37.491 | 1.00 | 31.18 | W | C |
| ATOM | 6531 | O | THR | W | 5 | 29.847 | 18.014 | −37.502 | 1.00 | 28.06 | W | O |
| ATOM | 6532 | N | GLN | W | 6 | 31.063 | 19.873 | −37.855 | 1.00 | 31.52 | W | N |
| ATOM | 6533 | CA | GLN | W | 6 | 29.974 | 20.723 | −38.318 | 1.00 | 27.48 | W | C |
| ATOM | 6534 | CB | GLN | W | 6 | 29.766 | 21.880 | −37.336 | 1.00 | 24.31 | W | C |
| ATOM | 6535 | CG | GLN | W | 6 | 28.948 | 21.546 | −36.107 | 1.00 | 29.05 | W | C |
| ATOM | 6536 | CD | GLN | W | 6 | 28.842 | 22.716 | −35.150 | 1.00 | 27.79 | W | C |
| ATOM | 6537 | OE1 | GLN | W | 6 | 29.636 | 22.839 | −34.220 | 1.00 | 26.71 | W | O |
| ATOM | 6538 | NE2 | GLN | W | 6 | 27.863 | 23.580 | −35.373 | 1.00 | 27.77 | W | N |
| ATOM | 6539 | C | GLN | W | 6 | 30.386 | 21.323 | −39.666 | 1.00 | 33.84 | W | C |
| ATOM | 6540 | O | GLN | W | 6 | 31.582 | 21.436 | −39.947 | 1.00 | 35.54 | W | O |
| ATOM | 6541 | N | GLU | W | 7 | 29.404 | 21.726 | −40.480 | 1.00 | 35.49 | W | N |
| ATOM | 6542 | CA | GLU | W | 7 | 29.670 | 22.364 | −41.775 | 1.00 | 32.38 | W | C |
| ATOM | 6543 | CB | GLU | W | 7 | 28.353 | 22.737 | −42.486 | 1.00 | 32.95 | W | C |
| ATOM | 6544 | CG | GLU | W | 7 | 27.408 | 21.574 | −42.829 | 1.00 | 35.05 | W | C |
| ATOM | 6545 | CD | GLU | W | 7 | 26.335 | 21.284 | −41.768 | 1.00 | 39.36 | W | C |
| ATOM | 6546 | OE1 | GLU | W | 7 | 26.119 | 22.097 | −40.839 | 1.00 | 41.83 | W | O |
| ATOM | 6547 | OE2 | GLU | W | 7 | 25.682 | 20.230 | −41.881 | 1.00 | 37.42 | W | O |
| ATOM | 6548 | C | GLU | W | 7 | 30.443 | 23.653 | −41.452 | 1.00 | 35.33 | W | C |
| ATOM | 6549 | O | GLU | W | 7 | 30.086 | 24.364 | −40.501 | 1.00 | 31.82 | W | O |
| ATOM | 6550 | N | SER | W | 8 | 31.486 | 23.952 | −42.226 | 1.00 | 29.98 | W | N |
| ATOM | 6551 | CA | SER | W | 8 | 32.289 | 25.155 | −41.994 | 1.00 | 34.72 | W | C |
| ATOM | 6552 | CB | SER | W | 8 | 33.370 | 25.315 | −43.067 | 1.00 | 34.62 | W | C |
| ATOM | 6553 | OG | SER | W | 8 | 34.413 | 24.372 | −42.904 | 1.00 | 45.33 | W | O |
| ATOM | 6554 | C | SER | W | 8 | 31.431 | 26.403 | −42.008 | 1.00 | 32.89 | W | C |
| ATOM | 6555 | O | SER | W | 8 | 31.382 | 27.149 | −41.037 | 1.00 | 34.30 | W | O |
| ATOM | 6556 | N | ALA | W | 9 | 30.714 | 26.598 | −43.107 | 1.00 | 33.82 | W | N |
| ATOM | 6557 | CA | ALA | W | 9 | 29.880 | 27.774 | −43.257 | 1.00 | 32.36 | W | C |
| ATOM | 6558 | CB | ALA | W | 9 | 30.690 | 28.892 | −43.886 | 1.00 | 28.42 | W | C |
| ATOM | 6559 | C | ALA | W | 9 | 28.676 | 27.479 | −44.117 | 1.00 | 33.85 | W | C |
| ATOM | 6560 | O | ALA | W | 9 | 28.715 | 26.597 | −44.972 | 1.00 | 38.54 | W | O |
| ATOM | 6561 | N | LEU | W | 11 | 27.597 | 28.214 | −43.886 | 1.00 | 28.61 | W | N |
| ATOM | 6562 | CA | LEU | W | 11 | 26.394 | 28.046 | −44.681 | 1.00 | 28.49 | W | C |
| ATOM | 6563 | CB | LEU | W | 11 | 25.374 | 27.136 | −44.004 | 1.00 | 31.31 | W | C |
| ATOM | 6564 | CG | LEU | W | 11 | 25.619 | 25.627 | −43.953 | 1.00 | 32.99 | W | C |
| ATOM | 6565 | CD1 | LEU | W | 11 | 24.457 | 25.028 | −43.188 | 1.00 | 31.91 | W | C |
| ATOM | 6566 | CD2 | LEU | W | 11 | 25.729 | 24.986 | −45.349 | 1.00 | 27.89 | W | C |
| ATOM | 6567 | C | LEU | W | 11 | 25.777 | 29.398 | −44.932 | 1.00 | 33.14 | W | C |
| ATOM | 6568 | O | LEU | W | 11 | 25.818 | 30.296 | −44.070 | 1.00 | 29.91 | W | O |
| ATOM | 6569 | N | THR | W | 12 | 25.241 | 29.558 | −46.137 | 1.00 | 30.14 | W | N |
| ATOM | 6570 | CA | THR | W | 12 | 24.610 | 30.809 | −46.503 | 1.00 | 34.54 | W | C |
| ATOM | 6571 | CB | THR | W | 12 | 25.402 | 31.552 | −47.607 | 1.00 | 33.15 | W | C |
| ATOM | 6572 | OG1 | THR | W | 12 | 24.517 | 32.383 | −48.368 | 1.00 | 42.40 | W | O |
| ATOM | 6573 | CG2 | THR | W | 12 | 26.131 | 30.584 | −48.507 | 1.00 | 44.93 | W | C |
| ATOM | 6574 | C | THR | W | 12 | 23.135 | 30.624 | −46.853 | 1.00 | 35.12 | W | C |
| ATOM | 6575 | O | THR | W | 12 | 22.756 | 29.703 | −47.571 | 1.00 | 30.30 | W | O |
| ATOM | 6576 | N | SER | W | 13 | 22.306 | 31.496 | −46.291 | 1.00 | 34.84 | W | N |
| ATOM | 6577 | CA | SER | W | 13 | 20.876 | 31.443 | −46.501 | 1.00 | 27.41 | W | C |
| ATOM | 6578 | CB | SER | W | 13 | 20.215 | 30.789 | −45.299 | 1.00 | 31.97 | W | C |
| ATOM | 6579 | OG | SER | W | 13 | 20.482 | 31.534 | −44.129 | 1.00 | 36.49 | W | O |
| ATOM | 6580 | C | SER | W | 13 | 20.328 | 32.845 | −46.697 | 1.00 | 29.90 | W | C |
| ATOM | 6581 | O | SER | W | 13 | 21.067 | 33.823 | −46.638 | 1.00 | 31.06 | W | O |
| ATOM | 6582 | N | SER | W | 14 | 19.025 | 32.934 | −46.913 | 1.00 | 23.84 | W | N |
| ATOM | 6583 | CA | SER | W | 14 | 18.374 | 34.208 | −47.140 | 1.00 | 28.57 | W | C |
| ATOM | 6584 | CB | SER | W | 14 | 17.797 | 34.255 | −48.567 | 1.00 | 33.54 | W | C |
| ATOM | 6585 | OG | SER | W | 14 | 18.762 | 33.906 | −49.557 | 1.00 | 40.07 | W | O |
| ATOM | 6586 | C | SER | W | 14 | 17.239 | 34.357 | −46.155 | 1.00 | 26.12 | W | C |
| ATOM | 6587 | O | SER | W | 14 | 16.747 | 33.376 | −45.616 | 1.00 | 24.65 | W | O |
| ATOM | 6588 | N | PRO | W | 15 | 16.828 | 35.596 | −45.878 | 1.00 | 24.72 | W | N |
| ATOM | 6589 | CD | PRO | W | 15 | 17.384 | 36.894 | −46.286 | 1.00 | 20.95 | W | C |
| ATOM | 6590 | CA | PRO | W | 15 | 15.720 | 35.754 | −44.937 | 1.00 | 25.10 | W | C |
| ATOM | 6591 | CB | PRO | W | 15 | 15.520 | 37.276 | −44.887 | 1.00 | 20.55 | W | C |
| ATOM | 6592 | CG | PRO | W | 15 | 16.191 | 37.791 | −46.135 | 1.00 | 24.76 | W | C |
| ATOM | 6593 | C | PRO | W | 15 | 14.469 | 34.999 | −45.435 | 1.00 | 24.19 | W | C |
| ATOM | 6594 | O | PRO | W | 15 | 14.159 | 35.017 | −46.624 | 1.00 | 29.93 | W | O |
| ATOM | 6595 | N | GLY | W | 16 | 13.813 | 34.282 | −44.523 | 1.00 | 23.27 | W | N |
| ATOM | 6596 | CA | GLY | W | 16 | 12.612 | 33.532 | −44.844 | 1.00 | 20.48 | W | C |
| ATOM | 6597 | C | GLY | W | 16 | 12.887 | 32.058 | −45.083 | 1.00 | 23.62 | W | C |
| ATOM | 6598 | O | GLY | W | 16 | 11.987 | 31.229 | −45.032 | 1.00 | 28.27 | W | O |
| ATOM | 6599 | N | GLU | W | 17 | 14.142 | 31.726 | −45.334 | 1.00 | 26.09 | W | N |
| ATOM | 6600 | CA | GLU | W | 17 | 14.531 | 30.356 | −45.611 | 1.00 | 27.12 | W | C |
| ATOM | 6601 | CB | GLU | W | 17 | 15.935 | 30.335 | −46.231 | 1.00 | 36.48 | W | C |
| ATOM | 6602 | CG | GLU | W | 17 | 16.398 | 28.967 | −46.760 | 1.00 | 44.55 | W | C |
| ATOM | 6603 | CD | GLU | W | 17 | 17.805 | 28.994 | −47.346 | 1.00 | 51.43 | W | C |
| ATOM | 6604 | OE1 | GLU | W | 17 | 18.152 | 29.961 | −48.068 | 1.00 | 51.23 | W | O |
| ATOM | 6605 | OE2 | GLU | W | 17 | 18.568 | 28.034 | −47.087 | 1.00 | 58.76 | W | O |
| ATOM | 6606 | C | GLU | W | 17 | 14.511 | 29.461 | −44.385 | 1.00 | 27.89 | W | C |
| ATOM | 6607 | O | GLU | W | 17 | 14.471 | 29.946 | −43.252 | 1.00 | 27.82 | W | O |
| ATOM | 6608 | N | THR | W | 18 | 14.455 | 28.154 | −44.637 | 1.00 | 21.53 | W | N |
| ATOM | 6609 | CA | THR | W | 18 | 14.497 | 27.138 | −43.593 | 1.00 | 23.66 | W | C |
| ATOM | 6610 | CB | THR | W | 18 | 13.349 | 26.079 | −43.714 | 1.00 | 24.69 | W | C |
| ATOM | 6611 | OG1 | THR | W | 18 | 12.136 | 26.638 | −43.210 | 1.00 | 25.55 | W | O |
| ATOM | 6612 | CG2 | THR | W | 18 | 13.655 | 24.809 | −42.905 | 1.00 | 15.73 | W | C |
| ATOM | 6613 | C | THR | W | 18 | 15.866 | 26.493 | −43.789 | 1.00 | 25.73 | W | C |
| ATOM | 6614 | O | THR | W | 18 | 16.156 | 25.943 | −44.851 | 1.00 | 23.82 | W | O |
| ATOM | 6615 | N | VAL | W | 19 | 16.721 | 26.635 | −42.780 | 1.00 | 29.25 | W | N |
| ATOM | 6616 | CA | VAL | W | 19 | 18.075 | 26.113 | −42.840 | 1.00 | 28.97 | W | C |
| ATOM | 6617 | CB | VAL | W | 19 | 19.091 | 27.272 | −42.849 | 1.00 | 29.01 | W | C |
| ATOM | 6618 | CG1 | VAL | W | 19 | 18.925 | 28.144 | −41.627 | 1.00 | 33.07 | W | C |
| ATOM | 6619 | CG2 | VAL | W | 19 | 20.497 | 26.731 | −42.970 | 1.00 | 32.28 | W | C |
| ATOM | 6620 | C | VAL | W | 19 | 18.393 | 25.061 | −41.766 | 1.00 | 27.58 | W | C |
| ATOM | 6621 | O | VAL | W | 19 | 17.921 | 25.137 | −40.629 | 1.00 | 33.78 | W | O |
| ATOM | 6622 | N | THR | W | 20 | 19.196 | 24.076 | −42.148 | 1.00 | 21.01 | W | N |
| ATOM | 6623 | CA | THR | W | 20 | 19.546 | 22.992 | −41.262 | 1.00 | 20.60 | W | C |
| ATOM | 6624 | CB | THR | W | 20 | 18.894 | 21.654 | −41.739 | 1.00 | 21.53 | W | C |
| ATOM | 6625 | OG1 | THR | W | 20 | 17.463 | 21.759 | −41.640 | 1.00 | 29.09 | W | O |
| ATOM | 6626 | CG2 | THR | W | 20 | 19.369 | 20.472 | −40.910 | 1.00 | 18.85 | W | C |
| ATOM | 6627 | C | THR | W | 20 | 21.034 | 22.799 | −41.084 | 1.00 | 24.12 | W | C |
| ATOM | 6628 | O | THR | W | 20 | 21.757 | 22.465 | −42.026 | 1.00 | 33.19 | W | O |
| ATOM | 6629 | N | LEU | W | 21 | 21.489 | 23.026 | −39.858 | 1.00 | 25.04 | W | N |
| ATOM | 6630 | CA | LEU | W | 21 | 22.891 | 22.840 | −39.518 | 1.00 | 20.75 | W | C |
| ATOM | 6631 | CB | LEU | W | 21 | 23.347 | 23.880 | −38.488 | 1.00 | 22.13 | W | C |
| ATOM | 6632 | CG | LEU | W | 21 | 22.988 | 25.352 | −38.673 | 1.00 | 22.92 | W | C |
| ATOM | 6633 | CD1 | LEU | W | 21 | 23.640 | 26.108 | −37.555 | 1.00 | 30.11 | W | C |
| ATOM | 6634 | CD2 | LEU | W | 21 | 23.489 | 25.898 | −39.977 | 1.00 | 22.50 | W | C |
| ATOM | 6635 | C | LEU | W | 21 | 22.942 | 21.459 | −38.887 | 1.00 | 21.97 | W | C |
| ATOM | 6636 | O | LEU | W | 21 | 22.011 | 21.077 | −38.176 | 1.00 | 24.39 | W | O |
| ATOM | 6637 | N | THR | W | 22 | 23.998 | 20.693 | −39.155 | 1.00 | 21.08 | W | N |
| ATOM | 6638 | CA | THR | W | 22 | 24.095 | 19.368 | −38.564 | 1.00 | 24.67 | W | C |
| ATOM | 6639 | CB | THR | W | 22 | 24.019 | 18.248 | −39.594 | 1.00 | 25.13 | W | C |
| ATOM | 6640 | OG1 | THR | W | 22 | 25.210 | 18.241 | −40.379 | 1.00 | 30.63 | W | O |
| ATOM | 6641 | CG2 | THR | W | 22 | 22.813 | 18.440 | −40.503 | 1.00 | 31.77 | W | C |
| ATOM | 6642 | C | THR | W | 22 | 25.347 | 19.191 | −37.738 | 1.00 | 25.60 | W | C |
| ATOM | 6643 | O | THR | W | 22 | 26.322 | 19.926 | −37.881 | 1.00 | 28.73 | W | O |
| ATOM | 6644 | N | CYS | W | 23 | 25.326 | 18.139 | −36.933 | 1.00 | 27.20 | W | N |
| ATOM | 6645 | CA | CYS | W | 23 | 26.399 | 17.822 | −36.015 | 1.00 | 28.28 | W | C |
| ATOM | 6646 | C | CYS | W | 23 | 26.670 | 16.321 | −36.020 | 1.00 | 24.76 | W | C |
| ATOM | 6647 | O | CYS | W | 23 | 25.827 | 15.538 | −35.593 | 1.00 | 29.22 | W | O |
| ATOM | 6648 | CB | CYS | W | 23 | 25.934 | 18.230 | −34.633 | 1.00 | 32.22 | W | C |
| ATOM | 6649 | SG | CYS | W | 23 | 27.157 | 18.106 | −33.312 | 1.00 | 35.47 | W | S |
| ATOM | 6650 | N | ARG | W | 24 | 27.855 | 15.927 | −36.461 | 1.00 | 24.88 | W | N |
| ATOM | 6651 | CA | ARG | W | 24 | 28.213 | 14.516 | −36.527 | 1.00 | 34.69 | W | C |
| ATOM | 6652 | CB | ARG | W | 24 | 28.662 | 14.148 | −37.961 | 1.00 | 35.97 | W | C |
| ATOM | 6653 | CG | ARG | W | 24 | 29.808 | 13.137 | −38.068 | 1.00 | 43.67 | W | C |
| ATOM | 6654 | CD | ARG | W | 24 | 29.858 | 12.458 | −39.433 | 1.00 | 44.68 | W | C |
| ATOM | 6655 | NE | ARG | W | 24 | 28.811 | 11.449 | −39.550 | 1.00 | 49.84 | W | N |
| ATOM | 6656 | CZ | ARG | W | 24 | 27.769 | 11.531 | −40.370 | 1.00 | 52.45 | W | C |
| ATOM | 6657 | NH1 | ARG | W | 24 | 27.617 | 12.575 | −41.172 | 1.00 | 56.40 | W | N |
| ATOM | 6658 | NH2 | ARG | W | 24 | 26.850 | 10.580 | −40.359 | 1.00 | 56.36 | W | N |
| ATOM | 6659 | C | ARG | W | 24 | 29.248 | 14.064 | −35.494 | 1.00 | 34.44 | W | C |
| ATOM | 6660 | O | ARG | W | 24 | 30.200 | 14.777 | −35.179 | 1.00 | 32.26 | W | O |
| ATOM | 6661 | N | SER | W | 25 | 29.021 | 12.868 | −34.967 | 1.00 | 36.09 | W | N |
| ATOM | 6662 | CA | SER | W | 25 | 29.905 | 12.248 | −33.997 | 1.00 | 36.55 | W | C |
| ATOM | 6663 | CB | SER | W | 25 | 29.084 | 11.529 | −32.929 | 1.00 | 35.49 | W | C |
| ATOM | 6664 | OG | SER | W | 25 | 29.915 | 10.827 | −32.015 | 1.00 | 32.68 | W | O |
| ATOM | 6665 | C | SER | W | 25 | 30.746 | 11.228 | −34.735 | 1.00 | 37.75 | W | C |
| ATOM | 6666 | O | SER | W | 25 | 30.222 | 10.469 | −35.547 | 1.00 | 43.76 | W | O |
| ATOM | 6667 | N | SER | W | 26 | 32.039 | 11.187 | −34.431 | 1.00 | 42.77 | W | N |
| ATOM | 6668 | CA | SER | W | 26 | 32.950 | 10.239 | −35.072 | 1.00 | 41.38 | W | C |
| ATOM | 6669 | CB | SER | W | 26 | 34.401 | 10.738 | −34.963 | 1.00 | 42.82 | W | C |
| ATOM | 6670 | OG | SER | W | 26 | 34.843 | 10.848 | −33.616 | 1.00 | 39.61 | W | O |
| ATOM | 6671 | C | SER | W | 26 | 32.836 | 8.800 | −34.535 | 1.00 | 43.59 | W | C |
| ATOM | 6672 | O | SER | W | 26 | 33.228 | 7.850 | −35.218 | 1.00 | 41.69 | W | O |
| ATOM | 6673 | N | THR | W | 27 | 32.293 | 8.642 | −33.325 | 1.00 | 39.96 | W | N |
| ATOM | 6674 | CA | THR | W | 27 | 32.147 | 7.323 | −32.698 | 1.00 | 42.04 | W | C |
| ATOM | 6675 | CB | THR | W | 27 | 31.880 | 7.418 | −31.193 | 1.00 | 42.74 | W | C |
| ATOM | 6676 | OG1 | THR | W | 27 | 30.716 | 8.226 | −30.961 | 1.00 | 49.11 | W | O |
| ATOM | 6677 | CG2 | THR | W | 27 | 33.073 | 7.992 | −30.473 | 1.00 | 48.30 | W | C |
| ATOM | 6678 | C | THR | W | 27 | 31.014 | 6.497 | −33.268 | 1.00 | 39.67 | W | C |
| ATOM | 6679 | O | THR | W | 27 | 31.058 | 5.263 | −33.250 | 1.00 | 41.74 | W | O |
| ATOM | 6680 | N | GLY | W | 27A | 29.974 | 7.182 | −33.725 | 1.00 | 36.82 | W | N |
| ATOM | 6681 | CA | GLY | W | 27A | 28.825 | 6.499 | −34.282 | 1.00 | 29.74 | W | C |
| ATOM | 6682 | C | GLY | W | 27A | 27.676 | 7.471 | −34.356 | 1.00 | 28.97 | W | C |
| ATOM | 6683 | O | GLY | W | 27A | 27.882 | 8.685 | −34.379 | 1.00 | 31.33 | W | O |
| ATOM | 6684 | N | ALA | W | 27B | 26.464 | 6.941 | −34.335 | 1.00 | 26.35 | W | N |
| ATOM | 6685 | CA | ALA | W | 27B | 25.264 | 7.762 | −34.410 | 1.00 | 31.03 | W | C |
| ATOM | 6686 | CB | ALA | W | 27B | 24.035 | 6.857 | −34.502 | 1.00 | 17.95 | W | C |
| ATOM | 6687 | C | ALA | W | 27B | 25.120 | 8.674 | −33.196 | 1.00 | 32.55 | W | C |
| ATOM | 6688 | O | ALA | W | 27B | 25.416 | 8.248 | −32.082 | 1.00 | 35.95 | W | O |
| ATOM | 6689 | N | VAL | W | 27C | 24.670 | 9.916 | −33.406 | 1.00 | 32.64 | W | N |
| ATOM | 6690 | CA | VAL | W | 27C | 24.444 | 10.825 | −32.284 | 1.00 | 30.92 | W | C |
| ATOM | 6691 | CB | VAL | W | 27C | 24.145 | 12.285 | −32.699 | 1.00 | 27.76 | W | C |
| ATOM | 6692 | CG1 | VAL | W | 27C | 23.845 | 13.111 | −31.456 | 1.00 | 25.01 | W | C |
| ATOM | 6693 | CG2 | VAL | W | 27C | 25.314 | 12.887 | −33.416 | 1.00 | 27.64 | W | C |
| ATOM | 6694 | C | VAL | W | 27C | 23.197 | 10.277 | −31.620 | 1.00 | 32.37 | W | C |
| ATOM | 6695 | O | VAL | W | 27C | 22.201 | 10.003 | −32.296 | 1.00 | 38.37 | W | O |
| ATOM | 6696 | N | THR | W | 28 | 23.243 | 10.157 | −30.299 | 1.00 | 35.35 | W | N |
| ATOM | 6697 | CA | THR | W | 28 | 22.138 | 9.604 | −29.527 | 1.00 | 32.14 | W | C |
| ATOM | 6698 | CB | THR | W | 28 | 22.597 | 8.266 | −28.930 | 1.00 | 40.12 | W | C |
| ATOM | 6699 | OG1 | THR | W | 28 | 21.538 | 7.311 | −29.006 | 1.00 | 46.85 | W | O |
| ATOM | 6700 | CG2 | THR | W | 28 | 23.078 | 8.428 | −27.514 | 1.00 | 36.82 | W | C |
| ATOM | 6701 | C | THR | W | 28 | 21.642 | 10.567 | −28.445 | 1.00 | 34.79 | W | C |
| ATOM | 6702 | O | THR | W | 28 | 22.296 | 11.580 | −28.171 | 1.00 | 33.94 | W | O |
| ATOM | 6703 | N | THR | W | 29 | 20.467 | 10.305 | −27.860 | 1.00 | 31.06 | W | N |
| ATOM | 6704 | CA | THR | W | 29 | 19.964 | 11.208 | −26.807 | 1.00 | 23.62 | W | C |
| ATOM | 6705 | CB | THR | W | 29 | 18.546 | 10.865 | −26.259 | 1.00 | 22.01 | W | C |
| ATOM | 6706 | OG1 | THR | W | 29 | 18.444 | 9.466 | −25.998 | 1.00 | 22.90 | W | O |
| ATOM | 6707 | CG2 | THR | W | 29 | 17.461 | 11.312 | −27.200 | 1.00 | 22.67 | W | C |
| ATOM | 6708 | C | THR | W | 29 | 20.911 | 11.252 | −25.606 | 1.00 | 23.12 | W | C |
| ATOM | 6709 | O | THR | W | 29 | 20.862 | 12.187 | −24.817 | 1.00 | 17.12 | W | O |
| ATOM | 6710 | N | SER | W | 30 | 21.732 | 10.219 | −25.453 | 1.00 | 16.88 | W | N |
| ATOM | 6711 | CA | SER | W | 30 | 22.705 | 10.154 | −24.369 | 1.00 | 27.98 | W | C |
| ATOM | 6712 | CB | SER | W | 30 | 23.294 | 8.746 | −24.247 | 1.00 | 27.53 | W | C |
| ATOM | 6713 | OG | SER | W | 30 | 22.263 | 7.779 | −24.093 | 1.00 | 37.88 | W | O |
| ATOM | 6714 | C | SER | W | 30 | 23.833 | 11.181 | −24.573 | 1.00 | 31.19 | W | C |
| ATOM | 6715 | O | SER | W | 30 | 24.582 | 11.488 | −23.629 | 1.00 | 31.68 | W | O |
| ATOM | 6716 | N | ASN | W | 31 | 23.940 | 11.712 | −25.793 | 1.00 | 21.73 | W | N |
| ATOM | 6717 | CA | ASN | W | 31 | 24.953 | 12.718 | −26.101 | 1.00 | 22.54 | W | C |
| ATOM | 6718 | CB | ASN | W | 31 | 25.312 | 12.709 | −27.581 | 1.00 | 19.06 | W | C |
| ATOM | 6719 | CG | ASN | W | 31 | 25.947 | 11.420 | −28.002 | 1.00 | 20.07 | W | C |
| ATOM | 6720 | OD1 | ASN | W | 31 | 25.505 | 10.795 | −28.957 | 1.00 | 29.28 | W | O |
| ATOM | 6721 | ND2 | ASN | W | 31 | 26.989 | 11.002 | −27.285 | 1.00 | 14.37 | W | N |
| ATOM | 6722 | C | ASN | W | 31 | 24.488 | 14.106 | −25.723 | 1.00 | 20.40 | W | C |
| ATOM | 6723 | O | ASN | W | 31 | 25.240 | 15.056 | −25.863 | 1.00 | 28.63 | W | O |
| ATOM | 6724 | N | TYR | W | 32 | 23.241 | 14.227 | −25.278 | 1.00 | 13.91 | W | N |
| ATOM | 6725 | CA | TYR | W | 32 | 22.686 | 15.517 | −24.864 | 1.00 | 16.68 | W | C |
| ATOM | 6726 | CB | TYR | W | 32 | 23.151 | 15.898 | −23.455 | 1.00 | 10.73 | W | C |
| ATOM | 6727 | CG | TYR | W | 32 | 22.419 | 15.246 | −22.325 | 1.00 | 13.11 | W | C |
| ATOM | 6728 | CD1 | TYR | W | 32 | 22.748 | 13.961 | −21.913 | 1.00 | 17.19 | W | C |
| ATOM | 6729 | CE1 | TYR | W | 32 | 22.101 | 13.367 | −20.854 | 1.00 | 17.55 | W | C |
| ATOM | 6730 | CD2 | TYR | W | 32 | 21.419 | 15.923 | −21.640 | 1.00 | 14.91 | W | C |
| ATOM | 6731 | CE2 | TYR | W | 32 | 20.769 | 15.342 | −20.576 | 1.00 | 17.84 | W | C |
| ATOM | 6732 | CZ | TYR | W | 32 | 21.114 | 14.056 | −20.191 | 1.00 | 20.20 | W | C |
| ATOM | 6733 | OH | TYR | W | 32 | 20.431 | 13.427 | −19.173 | 1.00 | 34.03 | W | O |
| ATOM | 6734 | C | TYR | W | 32 | 23.049 | 16.666 | −25.771 | 1.00 | 17.29 | W | C |
| ATOM | 6735 | O | TYR | W | 32 | 23.446 | 17.727 | −25.274 | 1.00 | 26.44 | W | O |
| ATOM | 6736 | N | ALA | W | 33 | 22.959 | 16.451 | −27.082 | 1.00 | 19.45 | W | N |
| ATOM | 6737 | CA | ALA | W | 33 | 23.281 | 17.482 | −28.067 | 1.00 | 17.05 | W | C |
| ATOM | 6738 | CB | ALA | W | 33 | 22.595 | 17.187 | −29.379 | 1.00 | 18.75 | W | C |
| ATOM | 6739 | C | ALA | W | 33 | 22.872 | 18.866 | −27.565 | 1.00 | 17.01 | W | C |
| ATOM | 6740 | O | ALA | W | 33 | 21.752 | 19.064 | −27.113 | 1.00 | 8.70 | W | O |
| ATOM | 6741 | N | ASN | W | 34 | 23.812 | 19.803 | −27.650 | 1.00 | 21.08 | W | N |
| ATOM | 6742 | CA | ASN | W | 34 | 23.651 | 21.180 | −27.182 | 1.00 | 20.85 | W | C |
| ATOM | 6743 | CB | ASN | W | 34 | 24.578 | 21.355 | −25.961 | 1.00 | 31.04 | W | C |
| ATOM | 6744 | CG | ASN | W | 34 | 24.229 | 22.552 | −25.114 | 1.00 | 32.15 | W | C |
| ATOM | 6745 | OD1 | ASN | W | 34 | 24.820 | 23.617 | −25.256 | 1.00 | 43.87 | W | O |
| ATOM | 6746 | ND2 | ASN | W | 34 | 23.295 | 22.374 | −24.195 | 1.00 | 39.86 | W | N |
| ATOM | 6747 | C | ASN | W | 34 | 24.081 | 22.140 | −28.306 | 1.00 | 18.18 | W | C |
| ATOM | 6748 | O | ASN | W | 34 | 25.087 | 21.914 | −28.970 | 1.00 | 25.14 | W | O |
| ATOM | 6749 | N | TRP | W | 35 | 23.307 | 23.193 | −28.532 | 1.00 | 19.89 | W | N |
| ATOM | 6750 | CA | TRP | W | 35 | 23.616 | 24.164 | −29.569 | 1.00 | 22.21 | W | C |
| ATOM | 6751 | CB | TRP | W | 35 | 22.533 | 24.199 | −30.664 | 1.00 | 22.73 | W | C |
| ATOM | 6752 | CG | TRP | W | 35 | 22.463 | 22.946 | −31.491 | 1.00 | 23.66 | W | C |
| ATOM | 6753 | CD2 | TRP | W | 35 | 23.126 | 22.689 | −32.736 | 1.00 | 22.67 | W | C |
| ATOM | 6754 | CE2 | TRP | W | 35 | 22.754 | 21.392 | −33.145 | 1.00 | 23.72 | W | C |
| ATOM | 6755 | CE3 | TRP | W | 35 | 23.998 | 23.428 | −33.548 | 1.00 | 25.36 | W | C |
| ATOM | 6756 | CD1 | TRP | W | 35 | 21.742 | 21.829 | −31.209 | 1.00 | 21.26 | W | C |
| ATOM | 6757 | NE1 | TRP | W | 35 | 21.910 | 20.892 | −32.191 | 1.00 | 21.94 | W | N |
| ATOM | 6758 | CZ2 | TRP | W | 35 | 23.220 | 20.814 | −34.331 | 1.00 | 21.05 | W | C |
| ATOM | 6759 | CZ3 | TRP | W | 35 | 24.461 | 22.855 | −34.728 | 1.00 | 21.27 | W | C |
| ATOM | 6760 | CH2 | TRP | W | 35 | 24.069 | 21.557 | −35.106 | 1.00 | 22.67 | W | C |
| ATOM | 6761 | C | TRP | W | 35 | 23.756 | 25.536 | −28.950 | 1.00 | 21.07 | W | C |
| ATOM | 6762 | O | TRP | W | 35 | 22.869 | 26.003 | −28.236 | 1.00 | 19.35 | W | O |
| ATOM | 6763 | N | VAL | W | 36 | 24.856 | 26.196 | −29.290 | 1.00 | 21.80 | W | N |
| ATOM | 6764 | CA | VAL | W | 36 | 25.170 | 27.517 | −28.769 | 1.00 | 21.59 | W | C |
| ATOM | 6765 | CB | VAL | W | 36 | 26.444 | 27.438 | −27.877 | 1.00 | 25.71 | W | C |
| ATOM | 6766 | CG1 | VAL | W | 36 | 26.833 | 28.790 | −27.373 | 1.00 | 27.23 | W | C |
| ATOM | 6767 | CG2 | VAL | W | 36 | 26.205 | 26.488 | −26.709 | 1.00 | 25.46 | W | C |
| ATOM | 6768 | C | VAL | W | 36 | 25.416 | 28.486 | −29.914 | 1.00 | 20.41 | W | C |
| ATOM | 6769 | O | VAL | W | 36 | 26.093 | 28.153 | −30.885 | 1.00 | 20.82 | W | O |
| ATOM | 6770 | N | GLN | W | 37 | 24.851 | 29.681 | −29.789 | 1.00 | 19.79 | W | N |
| ATOM | 6771 | CA | GLN | W | 37 | 24.989 | 30.748 | −30.779 | 1.00 | 23.13 | W | C |
| ATOM | 6772 | CB | GLN | W | 37 | 23.627 | 31.426 | −30.972 | 1.00 | 22.24 | W | C |
| ATOM | 6773 | CG | GLN | W | 37 | 23.619 | 32.694 | −31.801 | 1.00 | 30.84 | W | C |
| ATOM | 6774 | CD | GLN | W | 37 | 22.262 | 33.406 | −31.762 | 1.00 | 37.43 | W | C |
| ATOM | 6775 | OE1 | GLN | W | 37 | 22.030 | 34.284 | −30.926 | 1.00 | 43.96 | W | O |
| ATOM | 6776 | NE2 | GLN | W | 37 | 21.364 | 33.028 | −32.667 | 1.00 | 40.62 | W | N |
| ATOM | 6777 | C | GLN | W | 37 | 26.046 | 31.802 | −30.358 | 1.00 | 30.74 | W | C |
| ATOM | 6778 | O | GLN | W | 37 | 25.938 | 32.422 | −29.283 | 1.00 | 28.49 | W | O |
| ATOM | 6779 | N | GLU | W | 38 | 27.072 | 31.990 | −31.188 | 1.00 | 29.38 | W | N |
| ATOM | 6780 | CA | GLU | W | 38 | 28.091 | 32.996 | −30.906 | 1.00 | 34.06 | W | C |
| ATOM | 6781 | CB | GLU | W | 38 | 29.517 | 32.447 | −31.078 | 1.00 | 31.15 | W | C |
| ATOM | 6782 | CG | GLU | W | 38 | 30.600 | 33.524 | −30.817 | 1.00 | 30.85 | W | C |
| ATOM | 6783 | CD | GLU | W | 38 | 32.029 | 33.089 | −31.152 | 1.00 | 38.05 | W | C |
| ATOM | 6784 | OE1 | GLU | W | 38 | 32.339 | 31.875 | −31.168 | 1.00 | 42.91 | W | O |
| ATOM | 6785 | OE2 | GLU | W | 38 | 32.862 | 33.981 | −31.393 | 1.00 | 40.45 | W | O |
| ATOM | 6786 | C | GLU | W | 38 | 27.911 | 34.189 | −31.835 | 1.00 | 38.00 | W | C |
| ATOM | 6787 | O | GLU | W | 38 | 28.232 | 34.104 | −33.025 | 1.00 | 43.32 | W | O |
| ATOM | 6788 | N | LYS | W | 39 | 27.369 | 35.285 | −31.309 | 1.00 | 39.39 | W | N |
| ATOM | 6789 | CA | LYS | W | 39 | 27.191 | 36.493 | −32.107 | 1.00 | 41.75 | W | C |
| ATOM | 6790 | CB | LYS | W | 39 | 25.917 | 37.253 | −31.696 | 1.00 | 43.17 | W | C |
| ATOM | 6791 | CG | LYS | W | 39 | 24.614 | 36.544 | −32.085 | 1.00 | 40.97 | W | C |
| ATOM | 6792 | CD | LYS | W | 39 | 23.400 | 37.457 | −31.979 | 1.00 | 43.95 | W | C |
| ATOM | 6793 | CE | LYS | W | 39 | 23.457 | 38.571 | −33.020 | 1.00 | 51.83 | W | C |
| ATOM | 6794 | NZ | LYS | W | 39 | 22.421 | 39.629 | −32.809 | 1.00 | 51.16 | W | N |
| ATOM | 6795 | C | LYS | W | 39 | 28.440 | 37.384 | −31.999 | 1.00 | 46.97 | W | C |
| ATOM | 6796 | O | LYS | W | 39 | 29.301 | 37.163 | −31.140 | 1.00 | 46.71 | W | O |
| ATOM | 6797 | N | PRO | W | 40 | 28.594 | 38.358 | −32.918 | 1.00 | 52.01 | W | N |
| ATOM | 6798 | CD | PRO | W | 40 | 27.754 | 38.632 | −34.101 | 1.00 | 52.09 | W | C |
| ATOM | 6799 | CA | PRO | W | 40 | 29.749 | 39.261 | −32.902 | 1.00 | 51.37 | W | C |
| ATOM | 6800 | CB | PRO | W | 40 | 29.388 | 40.279 | −33.981 | 1.00 | 49.95 | W | C |
| ATOM | 6801 | CG | PRO | W | 40 | 28.679 | 39.455 | −34.968 | 1.00 | 46.92 | W | C |
| ATOM | 6802 | C | PRO | W | 40 | 29.974 | 39.940 | −31.550 | 1.00 | 47.88 | W | C |
| ATOM | 6803 | O | PRO | W | 40 | 29.023 | 40.394 | −30.912 | 1.00 | 38.18 | W | O |
| ATOM | 6804 | N | ASP | W | 41 | 31.252 | 40.038 | −31.174 | 1.00 | 50.81 | W | N |
| ATOM | 6805 | CA | ASP | W | 41 | 31.737 | 40.628 | −29.918 | 1.00 | 51.75 | W | C |
| ATOM | 6806 | CB | ASP | W | 41 | 30.906 | 41.866 | −29.499 | 1.00 | 56.37 | W | C |
| ATOM | 6807 | CG | ASP | W | 41 | 30.880 | 42.968 | −30.583 | 1.00 | 61.21 | W | C |
| ATOM | 6808 | OD1 | ASP | W | 41 | 31.780 | 42.977 | −31.459 | 1.00 | 62.09 | W | O |
| ATOM | 6809 | OD2 | ASP | W | 41 | 29.952 | 43.815 | −30.569 | 1.00 | 56.20 | W | O |
| ATOM | 6810 | C | ASP | W | 41 | 31.780 | 39.556 | −28.817 | 1.00 | 49.37 | W | C |
| ATOM | 6811 | O | ASP | W | 41 | 31.685 | 39.851 | −27.622 | 1.00 | 47.71 | W | O |
| ATOM | 6812 | N | HIS | W | 42 | 31.952 | 38.308 | −29.253 | 1.00 | 47.95 | W | N |
| ATOM | 6813 | CA | HIS | W | 42 | 32.019 | 37.140 | −28.376 | 1.00 | 46.89 | W | C |
| ATOM | 6814 | CB | HIS | W | 42 | 33.376 | 37.113 | −27.663 | 1.00 | 49.32 | W | C |
| ATOM | 6815 | CG | HIS | W | 42 | 34.527 | 36.826 | −28.581 | 1.00 | 52.40 | W | C |
| ATOM | 6816 | CD2 | HIS | W | 42 | 35.356 | 37.653 | −29.263 | 1.00 | 53.30 | W | C |
| ATOM | 6817 | ND1 | HIS | W | 42 | 34.909 | 35.543 | −28.918 | 1.00 | 54.72 | W | N |
| ATOM | 6818 | CE1 | HIS | W | 42 | 35.918 | 35.593 | −29.770 | 1.00 | 52.89 | W | C |
| ATOM | 6819 | NE2 | HIS | W | 42 | 36.209 | 36.862 | −29.995 | 1.00 | 53.57 | W | N |
| ATOM | 6820 | C | HIS | W | 42 | 30.844 | 36.983 | −27.388 | 1.00 | 47.10 | W | C |
| ATOM | 6821 | O | HIS | W | 42 | 31.041 | 36.796 | −26.189 | 1.00 | 50.89 | W | O |
| ATOM | 6822 | N | LEU | W | 43 | 29.622 | 37.019 | −27.911 | 1.00 | 45.93 | W | N |
| ATOM | 6823 | CA | LEU | W | 43 | 28.429 | 36.893 | −27.084 | 1.00 | 44.16 | W | C |
| ATOM | 6824 | CB | LEU | W | 43 | 27.442 | 38.023 | −27.407 | 1.00 | 48.25 | W | C |
| ATOM | 6825 | CG | LEU | W | 43 | 27.898 | 39.473 | −27.184 | 1.00 | 47.10 | W | C |
| ATOM | 6826 | CD1 | LEU | W | 43 | 26.878 | 40.446 | −27.771 | 1.00 | 46.76 | W | C |
| ATOM | 6827 | CD2 | LEU | W | 43 | 28.087 | 39.737 | −25.698 | 1.00 | 48.86 | W | C |
| ATOM | 6828 | C | LEU | W | 43 | 27.756 | 35.545 | −27.303 | 1.00 | 40.79 | W | C |
| ATOM | 6829 | O | LEU | W | 43 | 27.094 | 35.341 | −28.313 | 1.00 | 47.63 | W | O |
| ATOM | 6830 | N | PHE | W | 44 | 27.932 | 34.629 | −26.353 | 1.00 | 37.77 | W | N |
| ATOM | 6831 | CA | PHE | W | 44 | 27.337 | 33.299 | −26.438 | 1.00 | 27.16 | W | C |
| ATOM | 6832 | CB | PHE | W | 44 | 28.245 | 32.257 | −25.815 | 1.00 | 24.82 | W | C |
| ATOM | 6833 | CG | PHE | W | 44 | 29.511 | 32.049 | −26.555 | 1.00 | 24.72 | W | C |
| ATOM | 6834 | CD1 | PHE | W | 44 | 30.552 | 32.961 | −26.444 | 1.00 | 25.90 | W | C |
| ATOM | 6835 | CD2 | PHE | W | 44 | 29.667 | 30.949 | −27.383 | 1.00 | 26.81 | W | C |
| ATOM | 6836 | CE1 | PHE | W | 44 | 31.747 | 32.778 | −27.159 | 1.00 | 30.95 | W | C |
| ATOM | 6837 | CE2 | PHE | W | 44 | 30.847 | 30.750 | −28.102 | 1.00 | 29.41 | W | C |
| ATOM | 6838 | CZ | PHE | W | 44 | 31.894 | 31.673 | −27.988 | 1.00 | 29.10 | W | C |
| ATOM | 6839 | C | PHE | W | 44 | 25.974 | 33.229 | −25.778 | 1.00 | 34.63 | W | C |
| ATOM | 6840 | O | PHE | W | 44 | 25.755 | 33.830 | −24.723 | 1.00 | 35.16 | W | O |
| ATOM | 6841 | N | THR | W | 45 | 25.094 | 32.427 | −26.378 | 1.00 | 34.77 | W | N |
| ATOM | 6842 | CA | THR | W | 45 | 23.726 | 32.229 | −25.916 | 1.00 | 32.58 | W | C |
| ATOM | 6843 | CB | THR | W | 45 | 22.779 | 33.273 | −26.567 | 1.00 | 37.09 | W | C |
| ATOM | 6844 | OG1 | THR | W | 45 | 22.589 | 34.368 | −25.665 | 1.00 | 39.90 | W | O |
| ATOM | 6845 | CG2 | THR | W | 45 | 21.421 | 32.663 | −26.946 | 1.00 | 41.11 | W | C |
| ATOM | 6846 | C | THR | W | 45 | 23.283 | 30.827 | −26.304 | 1.00 | 34.84 | W | C |
| ATOM | 6847 | O | THR | W | 45 | 23.429 | 30.406 | −27.460 | 1.00 | 37.36 | W | O |
| ATOM | 6848 | N | GLY | W | 46 | 22.755 | 30.099 | −25.330 | 1.00 | 32.16 | W | N |
| ATOM | 6849 | CA | GLY | W | 46 | 22.286 | 28.758 | −25.597 | 1.00 | 30.95 | W | C |
| ATOM | 6850 | C | GLY | W | 46 | 21.084 | 28.784 | −26.513 | 1.00 | 23.81 | W | C |
| ATOM | 6851 | O | GLY | W | 46 | 20.248 | 29.682 | −26.442 | 1.00 | 27.33 | W | O |
| ATOM | 6852 | N | VAL | W | 47 | 21.003 | 27.816 | −27.408 | 1.00 | 24.05 | W | N |
| ATOM | 6853 | CA | VAL | W | 47 | 19.867 | 27.762 | −28.325 | 1.00 | 26.65 | W | C |
| ATOM | 6854 | CB | VAL | W | 47 | 20.352 | 27.719 | −29.797 | 1.00 | 25.31 | W | C |
| ATOM | 6855 | CG1 | VAL | W | 47 | 19.188 | 27.547 | −30.736 | 1.00 | 22.77 | W | C |
| ATOM | 6856 | CG2 | VAL | W | 47 | 21.116 | 28.993 | −30.136 | 1.00 | 19.29 | W | C |
| ATOM | 6857 | C | VAL | W | 47 | 19.006 | 26.540 | −27.999 | 1.00 | 26.85 | W | C |
| ATOM | 6858 | O | VAL | W | 47 | 17.832 | 26.660 | −27.638 | 1.00 | 27.91 | W | O |
| ATOM | 6859 | N | ILE | W | 48 | 19.653 | 25.381 | −28.032 | 1.00 | 24.27 | W | N |
| ATOM | 6860 | CA | ILE | W | 48 | 19.026 | 24.101 | −27.774 | 1.00 | 21.30 | W | C |
| ATOM | 6861 | CB | ILE | W | 48 | 19.022 | 23.258 | −29.072 | 1.00 | 23.77 | W | C |
| ATOM | 6862 | CG2 | ILE | W | 48 | 18.783 | 21.798 | −28.762 | 1.00 | 22.13 | W | C |
| ATOM | 6863 | CG1 | ILE | W | 48 | 18.044 | 23.837 | −30.104 | 1.00 | 24.75 | W | C |
| ATOM | 6864 | CD1 | ILE | W | 48 | 16.580 | 23.772 | −29.688 | 1.00 | 26.62 | W | C |
| ATOM | 6865 | C | ILE | W | 48 | 19.836 | 23.313 | −26.743 | 1.00 | 24.56 | W | C |
| ATOM | 6866 | O | ILE | W | 48 | 21.063 | 23.339 | −26.756 | 1.00 | 22.59 | W | O |
| ATOM | 6867 | N | GLY | W | 49 | 19.138 | 22.590 | −25.875 | 1.00 | 22.07 | W | N |
| ATOM | 6868 | CA | GLY | W | 49 | 19.807 | 21.746 | −24.908 | 1.00 | 17.76 | W | C |
| ATOM | 6869 | C | GLY | W | 49 | 19.049 | 20.432 | −24.885 | 1.00 | 23.96 | W | C |
| ATOM | 6870 | O | GLY | W | 49 | 17.891 | 20.391 | −25.303 | 1.00 | 28.60 | W | O |
| ATOM | 6871 | N | GLY | W | 50 | 19.685 | 19.359 | −24.430 | 1.00 | 21.54 | W | N |
| ATOM | 6872 | CA | GLY | W | 50 | 19.004 | 18.074 | −24.356 | 1.00 | 29.55 | W | C |
| ATOM | 6873 | C | GLY | W | 50 | 18.411 | 17.571 | −25.666 | 1.00 | 31.51 | W | C |
| ATOM | 6874 | O | GLY | W | 50 | 17.272 | 17.101 | −25.697 | 1.00 | 31.43 | W | O |
| ATOM | 6875 | N | THR | W | 51 | 19.195 | 17.697 | −26.738 | 1.00 | 33.74 | W | N |
| ATOM | 6876 | CA | THR | W | 51 | 18.848 | 17.279 | −28.098 | 1.00 | 23.97 | W | C |
| ATOM | 6877 | CB | THR | W | 51 | 18.386 | 15.811 | −28.173 | 1.00 | 28.10 | W | C |
| ATOM | 6878 | OG1 | THR | W | 51 | 19.344 | 14.963 | −27.533 | 1.00 | 26.32 | W | O |
| ATOM | 6879 | CG2 | THR | W | 51 | 18.234 | 15.381 | −29.646 | 1.00 | 26.70 | W | C |
| ATOM | 6880 | C | THR | W | 51 | 17.788 | 18.109 | −28.794 | 1.00 | 24.48 | W | C |
| ATOM | 6881 | O | THR | W | 51 | 18.006 | 18.596 | −29.899 | 1.00 | 22.99 | W | O |
| ATOM | 6882 | N | ASN | W | 52 | 16.661 | 18.314 | −28.134 | 1.00 | 23.58 | W | N |
| ATOM | 6883 | CA | ASN | W | 52 | 15.566 | 19.029 | −28.763 | 1.00 | 29.10 | W | C |
| ATOM | 6884 | CB | ASN | W | 52 | 14.585 | 18.028 | −29.391 | 1.00 | 33.67 | W | C |
| ATOM | 6885 | CG | ASN | W | 52 | 13.971 | 17.072 | −28.375 | 1.00 | 35.10 | W | C |
| ATOM | 6886 | OD1 | ASN | W | 52 | 13.282 | 16.126 | −28.754 | 1.00 | 44.97 | W | O |
| ATOM | 6887 | ND2 | ASN | W | 52 | 14.230 | 17.300 | −27.093 | 1.00 | 33.37 | W | N |
| ATOM | 6888 | C | ASN | W | 52 | 14.778 | 19.983 | −27.904 | 1.00 | 29.47 | W | C |
| ATOM | 6889 | O | ASN | W | 52 | 13.599 | 20.219 | −28.168 | 1.00 | 26.67 | W | O |
| ATOM | 6890 | N | ASN | W | 53 | 15.402 | 20.522 | −26.872 | 1.00 | 27.91 | W | N |
| ATOM | 6891 | CA | ASN | W | 53 | 14.697 | 21.462 | −26.023 | 1.00 | 25.69 | W | C |
| ATOM | 6892 | CB | ASN | W | 53 | 14.908 | 21.152 | −24.542 | 1.00 | 25.56 | W | C |
| ATOM | 6893 | CG | ASN | W | 53 | 14.607 | 19.726 | −24.202 | 1.00 | 22.89 | W | C |
| ATOM | 6894 | OD1 | ASN | W | 53 | 13.468 | 19.278 | −24.292 | 1.00 | 24.68 | W | O |
| ATOM | 6895 | ND2 | ASN | W | 53 | 15.635 | 18.990 | −23.826 | 1.00 | 30.36 | W | N |
| ATOM | 6896 | C | ASN | W | 53 | 15.178 | 22.860 | −26.299 | 1.00 | 26.10 | W | C |
| ATOM | 6897 | O | ASN | W | 53 | 16.374 | 23.172 | −26.190 | 1.00 | 31.95 | W | O |
| ATOM | 6898 | N | ARG | W | 54 | 14.239 | 23.698 | −26.679 | 1.00 | 20.90 | W | N |
| ATOM | 6899 | CA | ARG | W | 54 | 14.524 | 25.092 | −26.928 | 1.00 | 28.08 | W | C |
| ATOM | 6900 | CB | ARG | W | 54 | 13.319 | 25.714 | −27.609 | 1.00 | 23.63 | W | C |
| ATOM | 6901 | CG | ARG | W | 54 | 13.556 | 27.059 | −28.196 | 1.00 | 37.34 | W | C |
| ATOM | 6902 | CD | ARG | W | 54 | 12.835 | 27.164 | −29.534 | 1.00 | 39.57 | W | C |
| ATOM | 6903 | NE | ARG | W | 54 | 11.392 | 27.011 | −29.398 | 1.00 | 44.12 | W | N |
| ATOM | 6904 | CZ | ARG | W | 54 | 10.721 | 25.897 | −29.675 | 1.00 | 47.52 | W | C |
| ATOM | 6905 | NH1 | ARG | W | 54 | 11.361 | 24.815 | −30.117 | 1.00 | 48.19 | W | N |
| ATOM | 6906 | NH2 | ARG | W | 54 | 9.410 | 25.857 | −29.464 | 1.00 | 50.30 | W | N |
| ATOM | 6907 | C | ARG | W | 54 | 14.728 | 25.727 | −25.552 | 1.00 | 29.68 | W | C |
| ATOM | 6908 | O | ARG | W | 54 | 13.920 | 25.515 | −24.632 | 1.00 | 28.97 | W | O |
| ATOM | 6909 | N | ALA | W | 55 | 15.831 | 26.448 | −25.396 | 1.00 | 27.07 | W | N |
| ATOM | 6910 | CA | ALA | W | 55 | 16.140 | 27.124 | −24.131 | 1.00 | 29.91 | W | C |
| ATOM | 6911 | CB | ALA | W | 55 | 17.551 | 27.716 | −24.196 | 1.00 | 30.24 | W | C |
| ATOM | 6912 | C | ALA | W | 55 | 15.107 | 28.234 | −23.923 | 1.00 | 27.66 | W | C |
| ATOM | 6913 | O | ALA | W | 55 | 14.706 | 28.888 | −24.885 | 1.00 | 28.94 | W | O |
| ATOM | 6914 | N | PRO | W | 56 | 14.664 | 28.463 | −22.673 | 1.00 | 23.78 | W | N |
| ATOM | 6915 | CD | PRO | W | 56 | 15.064 | 27.793 | −21.426 | 1.00 | 21.54 | W | C |
| ATOM | 6916 | CA | PRO | W | 56 | 13.667 | 29.515 | −22.421 | 1.00 | 26.79 | W | C |
| ATOM | 6917 | CB | PRO | W | 56 | 13.609 | 29.566 | −20.900 | 1.00 | 26.07 | W | C |
| ATOM | 6918 | CG | PRO | W | 56 | 13.934 | 28.137 | −20.509 | 1.00 | 25.14 | W | C |
| ATOM | 6919 | C | PRO | W | 56 | 14.080 | 30.863 | −23.032 | 1.00 | 30.33 | W | C |
| ATOM | 6920 | O | PRO | W | 56 | 15.252 | 31.255 | −22.960 | 1.00 | 36.65 | W | O |
| ATOM | 6921 | N | GLY | W | 57 | 13.137 | 31.521 | −23.706 | 1.00 | 32.47 | W | N |
| ATOM | 6922 | CA | GLY | W | 57 | 13.428 | 32.804 | −24.329 | 1.00 | 34.41 | W | C |
| ATOM | 6923 | C | GLY | W | 57 | 13.684 | 32.718 | −25.822 | 1.00 | 38.47 | W | C |
| ATOM | 6924 | O | GLY | W | 57 | 13.413 | 33.667 | −26.563 | 1.00 | 41.44 | W | O |
| ATOM | 6925 | N | VAL | W | 58 | 14.189 | 31.568 | −26.262 | 1.00 | 37.97 | W | N |
| ATOM | 6926 | CA | VAL | W | 58 | 14.501 | 31.331 | −27.665 | 1.00 | 39.14 | W | C |
| ATOM | 6927 | CB | VAL | W | 58 | 15.388 | 30.068 | −27.809 | 1.00 | 40.40 | W | C |
| ATOM | 6928 | CG1 | VAL | W | 58 | 15.586 | 29.697 | −29.284 | 1.00 | 38.32 | W | C |
| ATOM | 6929 | CG2 | VAL | W | 58 | 16.733 | 30.311 | −27.149 | 1.00 | 33.46 | W | C |
| ATOM | 6930 | C | VAL | W | 58 | 13.242 | 31.221 | −28.543 | 1.00 | 40.17 | W | C |
| ATOM | 6931 | O | VAL | W | 58 | 12.305 | 30.483 | −28.209 | 1.00 | 41.76 | W | O |
| ATOM | 6932 | N | PRO | W | 59 | 13.216 | 31.955 | −29.681 | 1.00 | 38.36 | W | N |
| ATOM | 6933 | CD | PRO | W | 59 | 14.348 | 32.794 | −30.114 | 1.00 | 34.83 | W | C |
| ATOM | 6934 | CA | PRO | W | 59 | 12.127 | 32.017 | −30.672 | 1.00 | 38.32 | W | C |
| ATOM | 6935 | CB | PRO | W | 59 | 12.755 | 32.810 | −31.812 | 1.00 | 39.48 | W | C |
| ATOM | 6936 | CG | PRO | W | 59 | 13.705 | 33.712 | −31.111 | 1.00 | 38.31 | W | C |
| ATOM | 6937 | C | PRO | W | 59 | 11.705 | 30.642 | −31.155 | 1.00 | 37.43 | W | C |
| ATOM | 6938 | O | PRO | W | 59 | 12.548 | 29.774 | −31.339 | 1.00 | 41.07 | W | O |
| ATOM | 6939 | N | ALA | W | 60 | 10.408 | 30.464 | −31.405 | 1.00 | 39.51 | W | N |
| ATOM | 6940 | CA | ALA | W | 60 | 9.872 | 29.179 | −31.845 | 1.00 | 33.89 | W | C |
| ATOM | 6941 | CB | ALA | W | 60 | 8.359 | 29.228 | −31.862 | 1.00 | 32.65 | W | C |
| ATOM | 6942 | C | ALA | W | 60 | 10.423 | 28.686 | −33.188 | 1.00 | 31.27 | W | C |
| ATOM | 6943 | O | ALA | W | 60 | 10.274 | 27.511 | −33.527 | 1.00 | 36.95 | W | O |
| ATOM | 6944 | N | ARG | W | 61 | 11.082 | 29.566 | −33.936 | 1.00 | 30.82 | W | N |
| ATOM | 6945 | CA | ARG | W | 61 | 11.659 | 29.184 | −35.232 | 1.00 | 35.47 | W | C |
| ATOM | 6946 | CB | ARG | W | 61 | 11.967 | 30.418 | −36.082 | 1.00 | 34.44 | W | C |
| ATOM | 6947 | CG | ARG | W | 61 | 13.049 | 31.330 | −35.543 | 1.00 | 38.40 | W | C |
| ATOM | 6948 | CD | ARG | W | 61 | 13.257 | 32.506 | −36.491 | 1.00 | 37.56 | W | C |
| ATOM | 6949 | NE | ARG | W | 61 | 14.315 | 33.403 | −36.033 | 1.00 | 43.99 | W | N |
| ATOM | 6950 | CZ | ARG | W | 61 | 14.146 | 34.360 | −35.123 | 1.00 | 46.32 | W | C |
| ATOM | 6951 | NH1 | ARG | W | 61 | 12.950 | 34.547 | −34.569 | 1.00 | 40.18 | W | N |
| ATOM | 6952 | NH2 | ARG | W | 61 | 15.179 | 35.118 | −34.759 | 1.00 | 41.72 | W | N |
| ATOM | 6953 | C | ARG | W | 61 | 12.900 | 28.281 | −35.136 | 1.00 | 33.21 | W | C |
| ATOM | 6954 | O | ARG | W | 61 | 13.341 | 27.703 | −36.129 | 1.00 | 38.79 | W | O |
| ATOM | 6955 | N | PHE | W | 62 | 13.482 | 28.198 | −33.946 | 1.00 | 28.19 | W | N |
| ATOM | 6956 | CA | PHE | W | 62 | 14.637 | 27.340 | −33.715 | 1.00 | 24.82 | W | C |
| ATOM | 6957 | CB | PHE | W | 62 | 15.603 | 27.971 | −32.702 | 1.00 | 17.90 | W | C |
| ATOM | 6958 | CG | PHE | W | 62 | 16.379 | 29.122 | −33.242 | 1.00 | 21.06 | W | C |
| ATOM | 6959 | CD1 | PHE | W | 62 | 17.527 | 28.907 | −33.999 | 1.00 | 20.78 | W | C |
| ATOM | 6960 | CD2 | PHE | W | 62 | 15.983 | 30.427 | −32.981 | 1.00 | 18.69 | W | C |
| ATOM | 6961 | CE1 | PHE | W | 62 | 18.270 | 29.973 | −34.487 | 1.00 | 20.38 | W | C |
| ATOM | 6962 | CE2 | PHE | W | 62 | 16.720 | 31.501 | −33.465 | 1.00 | 16.26 | W | C |
| ATOM | 6963 | CZ | PHE | W | 62 | 17.866 | 31.272 | −34.221 | 1.00 | 18.11 | W | C |
| ATOM | 6964 | C | PHE | W | 62 | 14.130 | 26.025 | −33.131 | 1.00 | 21.94 | W | C |
| ATOM | 6965 | O | PHE | W | 62 | 13.218 | 26.023 | −32.310 | 1.00 | 25.97 | W | O |
| ATOM | 6966 | N | SER | W | 63 | 14.699 | 24.910 | −33.570 | 1.00 | 17.58 | W | N |
| ATOM | 6967 | CA | SER | W | 63 | 14.317 | 23.613 | −33.046 | 1.00 | 22.42 | W | C |
| ATOM | 6968 | CB | SER | W | 63 | 12.966 | 23.121 | −33.605 | 1.00 | 25.44 | W | C |
| ATOM | 6969 | OG | SER | W | 63 | 13.040 | 22.715 | −34.960 | 1.00 | 38.41 | W | O |
| ATOM | 6970 | C | SER | W | 63 | 15.447 | 22.658 | −33.335 | 1.00 | 18.11 | W | C |
| ATOM | 6971 | O | SER | W | 63 | 16.209 | 22.864 | −34.275 | 1.00 | 26.46 | W | O |
| ATOM | 6972 | N | GLY | W | 64 | 15.578 | 21.646 | −32.487 | 1.00 | 17.96 | W | N |
| ATOM | 6973 | CA | GLY | W | 64 | 16.644 | 20.681 | −32.631 | 1.00 | 7.37 | W | C |
| ATOM | 6974 | C | GLY | W | 64 | 16.123 | 19.276 | −32.710 | 1.00 | 21.53 | W | C |
| ATOM | 6975 | O | GLY | W | 64 | 14.992 | 19.006 | −32.266 | 1.00 | 19.95 | W | O |
| ATOM | 6976 | N | SER | W | 65 | 16.937 | 18.367 | −33.249 | 1.00 | 14.75 | W | N |
| ATOM | 6977 | CA | SER | W | 65 | 16.489 | 16.997 | −33.380 | 1.00 | 20.22 | W | C |
| ATOM | 6978 | CB | SER | W | 65 | 15.340 | 16.930 | −34.402 | 1.00 | 26.92 | W | C |
| ATOM | 6979 | OG | SER | W | 65 | 15.766 | 17.340 | −35.699 | 1.00 | 24.22 | W | O |
| ATOM | 6980 | C | SER | W | 65 | 17.586 | 16.085 | −33.858 | 1.00 | 25.16 | W | C |
| ATOM | 6981 | O | SER | W | 65 | 18.743 | 16.478 | −33.975 | 1.00 | 23.78 | W | O |
| ATOM | 6982 | N | LEU | W | 66 | 17.194 | 14.841 | −34.103 | 1.00 | 31.14 | W | N |
| ATOM | 6983 | CA | LEU | W | 66 | 18.090 | 13.831 | −34.638 | 1.00 | 36.79 | W | C |
| ATOM | 6984 | CB | LEU | W | 66 | 18.153 | 12.604 | −33.723 | 1.00 | 34.61 | W | C |
| ATOM | 6985 | CG | LEU | W | 66 | 18.719 | 12.953 | −32.329 | 1.00 | 41.62 | W | C |
| ATOM | 6986 | CD1 | LEU | W | 66 | 18.659 | 11.564 | −31.517 | 1.00 | 42.42 | W | C |
| ATOM | 6987 | CD2 | LEU | W | 66 | 20.162 | 13.375 | −32.414 | 1.00 | 40.07 | W | C |
| ATOM | 6988 | C | LEU | W | 66 | 17.510 | 13.456 | −36.010 | 1.00 | 34.08 | W | C |
| ATOM | 6989 | O | LEU | W | 66 | 16.331 | 13.128 | −36.127 | 1.00 | 37.57 | W | O |
| ATOM | 6990 | N | ILE | W | 67 | 18.312 | 13.648 | −37.049 | 1.00 | 35.43 | W | N |
| ATOM | 6991 | CA | ILE | W | 67 | 17.939 | 13.327 | −38.419 | 1.00 | 36.37 | W | C |
| ATOM | 6992 | CB | ILE | W | 67 | 17.876 | 14.588 | −39.342 | 1.00 | 37.59 | W | C |
| ATOM | 6993 | CG2 | ILE | W | 67 | 17.570 | 14.167 | −40.779 | 1.00 | 37.61 | W | C |
| ATOM | 6994 | CG1 | ILE | W | 67 | 16.784 | 15.558 | −38.859 | 1.00 | 36.89 | W | C |
| ATOM | 6995 | CD1 | ILE | W | 67 | 16.845 | 16.941 | −39.489 | 1.00 | 27.69 | W | C |
| ATOM | 6996 | C | ILE | W | 67 | 19.057 | 12.415 | −38.880 | 1.00 | 35.32 | W | C |
| ATOM | 6997 | O | ILE | W | 67 | 20.211 | 12.834 | −38.998 | 1.00 | 31.00 | W | O |
| ATOM | 6998 | N | GLY | W | 68 | 18.712 | 11.156 | −39.116 | 1.00 | 40.66 | W | N |
| ATOM | 6999 | CA | GLY | W | 68 | 19.710 | 10.191 | −39.523 | 1.00 | 39.97 | W | C |
| ATOM | 7000 | C | GLY | W | 68 | 20.536 | 9.898 | −38.290 | 1.00 | 41.21 | W | C |
| ATOM | 7001 | O | GLY | W | 68 | 19.988 | 9.742 | −37.192 | 1.00 | 39.96 | W | O |
| ATOM | 7002 | N | ASP | W | 69 | 21.852 | 9.855 | −38.447 | 1.00 | 44.38 | W | N |
| ATOM | 7003 | CA | ASP | W | 69 | 22.715 | 9.595 | −37.303 | 1.00 | 42.89 | W | C |
| ATOM | 7004 | CB | ASP | W | 69 | 23.668 | 8.420 | −37.588 | 1.00 | 49.15 | W | C |
| ATOM | 7005 | CG | ASP | W | 69 | 24.074 | 8.325 | −39.040 | 1.00 | 51.27 | W | C |
| ATOM | 7006 | OD1 | ASP | W | 69 | 24.223 | 9.382 | −39.684 | 1.00 | 58.31 | W | O |
| ATOM | 7007 | OD2 | ASP | W | 69 | 24.249 | 7.190 | −39.537 | 1.00 | 55.97 | W | O |
| ATOM | 7008 | C | ASP | W | 69 | 23.450 | 10.852 | −36.829 | 1.00 | 36.63 | W | C |
| ATOM | 7009 | O | ASP | W | 69 | 24.517 | 10.780 | −36.228 | 1.00 | 36.10 | W | O |
| ATOM | 7010 | N | LYS | W | 70 | 22.805 | 11.998 | −37.026 | 1.00 | 33.83 | W | N |
| ATOM | 7011 | CA | LYS | W | 70 | 23.362 | 13.296 | −36.652 | 1.00 | 34.83 | W | C |
| ATOM | 7012 | CB | LYS | W | 70 | 23.749 | 14.074 | −37.918 | 1.00 | 33.81 | W | C |
| ATOM | 7013 | CG | LYS | W | 70 | 24.754 | 13.370 | −38.826 | 1.00 | 39.69 | W | C |
| ATOM | 7014 | CD | LYS | W | 70 | 25.344 | 14.303 | −39.870 | 1.00 | 34.42 | W | C |
| ATOM | 7015 | CE | LYS | W | 70 | 24.316 | 14.735 | −40.885 | 1.00 | 36.54 | W | C |
| ATOM | 7016 | NZ | LYS | W | 70 | 24.909 | 15.752 | −41.783 | 1.00 | 29.79 | W | N |
| ATOM | 7017 | C | LYS | W | 70 | 22.365 | 14.157 | −35.892 | 1.00 | 32.86 | W | C |
| ATOM | 7018 | O | LYS | W | 70 | 21.164 | 14.015 | −36.068 | 1.00 | 31.47 | W | O |
| ATOM | 7019 | N | ALA | W | 71 | 22.873 | 15.065 | −35.063 | 1.00 | 32.26 | W | N |
| ATOM | 7020 | CA | ALA | W | 71 | 22.015 | 16.001 | −34.338 | 1.00 | 30.53 | W | C |
| ATOM | 7021 | CB | ALA | W | 71 | 22.692 | 16.464 | −33.073 | 1.00 | 30.12 | W | C |
| ATOM | 7022 | C | ALA | W | 71 | 21.839 | 17.176 | −35.291 | 1.00 | 26.27 | W | C |
| ATOM | 7023 | O | ALA | W | 71 | 22.729 | 17.452 | −36.092 | 1.00 | 25.31 | W | O |
| ATOM | 7024 | N | ALA | W | 72 | 20.720 | 17.885 | −35.191 | 1.00 | 22.31 | W | N |
| ATOM | 7025 | CA | ALA | W | 72 | 20.473 | 19.005 | −36.085 | 1.00 | 20.91 | W | C |
| ATOM | 7026 | CB | ALA | W | 72 | 19.715 | 18.525 | −37.300 | 1.00 | 22.50 | W | C |
| ATOM | 7027 | C | ALA | W | 72 | 19.727 | 20.168 | −35.470 | 1.00 | 19.50 | W | C |
| ATOM | 7028 | O | ALA | W | 72 | 18.898 | 19.990 | −34.586 | 1.00 | 28.28 | W | O |
| ATOM | 7029 | N | LEU | W | 73 | 20.030 | 21.359 | −35.973 | 1.00 | 19.96 | W | N |
| ATOM | 7030 | CA | LEU | W | 73 | 19.393 | 22.604 | −35.561 | 1.00 | 21.37 | W | C |
| ATOM | 7031 | CB | LEU | W | 73 | 20.447 | 23.611 | −35.087 | 1.00 | 25.20 | W | C |
| ATOM | 7032 | CG | LEU | W | 73 | 19.985 | 25.060 | −34.902 | 1.00 | 24.48 | W | C |
| ATOM | 7033 | CD1 | LEU | W | 73 | 18.923 | 25.139 | −33.810 | 1.00 | 23.96 | W | C |
| ATOM | 7034 | CD2 | LEU | W | 73 | 21.183 | 25.942 | −34.561 | 1.00 | 19.81 | W | C |
| ATOM | 7035 | C | LEU | W | 73 | 18.678 | 23.158 | −36.805 | 1.00 | 24.45 | W | C |
| ATOM | 7036 | O | LEU | W | 73 | 19.316 | 23.466 | −37.824 | 1.00 | 25.99 | W | O |
| ATOM | 7037 | N | THR | W | 74 | 17.355 | 23.264 | −36.738 | 1.00 | 18.90 | W | N |
| ATOM | 7038 | CA | THR | W | 74 | 16.594 | 23.755 | −37.879 | 1.00 | 20.73 | W | C |
| ATOM | 7039 | CB | THR | W | 74 | 15.380 | 22.815 | −38.227 | 1.00 | 19.39 | W | C |
| ATOM | 7040 | OG1 | THR | W | 74 | 15.842 | 21.481 | −38.498 | 1.00 | 18.34 | W | O |
| ATOM | 7041 | CG2 | THR | W | 74 | 14.630 | 23.338 | −39.452 | 1.00 | 10.68 | W | C |
| ATOM | 7042 | C | THR | W | 74 | 16.093 | 25.149 | −37.562 | 1.00 | 22.04 | W | C |
| ATOM | 7043 | O | THR | W | 74 | 15.623 | 25.410 | −36.463 | 1.00 | 28.07 | W | O |
| ATOM | 7044 | N | ILE | W | 75 | 16.262 | 26.061 | −38.507 | 1.00 | 26.78 | W | N |
| ATOM | 7045 | CA | ILE | W | 75 | 15.804 | 27.431 | −38.332 | 1.00 | 28.89 | W | C |
| ATOM | 7046 | CB | ILE | W | 75 | 16.963 | 28.450 | −38.517 | 1.00 | 33.40 | W | C |
| ATOM | 7047 | CG2 | ILE | W | 75 | 16.472 | 29.869 | −38.190 | 1.00 | 29.47 | W | C |
| ATOM | 7048 | CG1 | ILE | W | 75 | 18.147 | 28.053 | −37.625 | 1.00 | 27.64 | W | C |
| ATOM | 7049 | CD1 | ILE | W | 75 | 19.327 | 28.974 | −37.697 | 1.00 | 29.31 | W | C |
| ATOM | 7050 | C | ILE | W | 75 | 14.733 | 27.614 | −39.403 | 1.00 | 29.95 | W | C |
| ATOM | 7051 | O | ILE | W | 75 | 15.025 | 27.597 | −40.599 | 1.00 | 26.58 | W | O |
| ATOM | 7052 | N | THR | W | 76 | 13.487 | 27.719 | −38.952 | 1.00 | 26.34 | W | N |
| ATOM | 7053 | CA | THR | W | 76 | 12.333 | 27.846 | −39.824 | 1.00 | 21.39 | W | C |
| ATOM | 7054 | CB | THR | W | 76 | 11.140 | 27.080 | −39.225 | 1.00 | 23.30 | W | C |
| ATOM | 7055 | OG1 | THR | W | 76 | 11.542 | 25.730 | −38.964 | 1.00 | 24.45 | W | O |
| ATOM | 7056 | CG2 | THR | W | 76 | 9.952 | 27.077 | −40.183 | 1.00 | 20.66 | W | C |
| ATOM | 7057 | C | THR | W | 76 | 11.960 | 29.293 | −40.042 | 1.00 | 26.17 | W | C |
| ATOM | 7058 | O | THR | W | 76 | 11.167 | 29.863 | −39.289 | 1.00 | 27.90 | W | O |
| ATOM | 7059 | N | GLY | W | 77 | 12.497 | 29.871 | −41.111 | 1.00 | 31.21 | W | N |
| ATOM | 7060 | CA | GLY | W | 77 | 12.224 | 31.265 | −41.401 | 1.00 | 33.02 | W | C |
| ATOM | 7061 | C | GLY | W | 77 | 13.348 | 32.099 | −40.819 | 1.00 | 35.71 | W | C |
| ATOM | 7062 | O | GLY | W | 77 | 13.131 | 32.990 | −39.988 | 1.00 | 40.25 | W | O |
| ATOM | 7063 | N | ALA | W | 78 | 14.560 | 31.778 | −41.253 | 1.00 | 29.88 | W | N |
| ATOM | 7064 | CA | ALA | W | 78 | 15.771 | 32.454 | −40.823 | 1.00 | 30.79 | W | C |
| ATOM | 7065 | CB | ALA | W | 78 | 16.924 | 32.000 | −41.692 | 1.00 | 28.23 | W | C |
| ATOM | 7066 | C | ALA | W | 78 | 15.649 | 33.964 | −40.888 | 1.00 | 29.84 | W | C |
| ATOM | 7067 | O | ALA | W | 78 | 15.306 | 34.508 | −41.920 | 1.00 | 28.78 | W | O |
| ATOM | 7068 | N | GLN | W | 79 | 15.902 | 34.644 | −39.780 | 1.00 | 34.66 | W | N |
| ATOM | 7069 | CA | GLN | W | 79 | 15.833 | 36.102 | −39.788 | 1.00 | 41.27 | W | C |
| ATOM | 7070 | CB | GLN | W | 79 | 15.094 | 36.624 | −38.554 | 1.00 | 41.88 | W | C |
| ATOM | 7071 | CG | GLN | W | 79 | 13.584 | 36.429 | −38.592 | 1.00 | 48.00 | W | C |
| ATOM | 7072 | CD | GLN | W | 79 | 12.941 | 37.103 | −39.791 | 1.00 | 55.87 | W | C |
| ATOM | 7073 | OE1 | GLN | W | 79 | 12.674 | 36.457 | −40.806 | 1.00 | 60.13 | W | O |
| ATOM | 7074 | NE2 | GLN | W | 79 | 12.686 | 38.412 | −39.682 | 1.00 | 57.55 | W | N |
| ATOM | 7075 | C | GLN | W | 79 | 17.252 | 36.664 | −39.868 | 1.00 | 38.64 | W | C |
| ATOM | 7076 | O | GLN | W | 79 | 18.222 | 35.909 | −39.795 | 1.00 | 40.19 | W | O |
| ATOM | 7077 | N | THR | W | 80 | 17.379 | 37.967 | −40.096 | 1.00 | 38.80 | W | N |
| ATOM | 7078 | CA | THR | W | 80 | 18.697 | 38.590 | −40.171 | 1.00 | 37.34 | W | C |
| ATOM | 7079 | CB | THR | W | 80 | 18.598 | 40.050 | −40.683 | 1.00 | 35.96 | W | C |
| ATOM | 7080 | OG1 | THR | W | 80 | 18.976 | 40.081 | −42.070 | 1.00 | 38.15 | W | O |
| ATOM | 7081 | CG2 | THR | W | 80 | 19.480 | 41.004 | −39.872 | 1.00 | 32.63 | W | C |
| ATOM | 7082 | C | THR | W | 80 | 19.397 | 38.503 | −38.814 | 1.00 | 41.23 | W | C |
| ATOM | 7083 | O | THR | W | 80 | 20.596 | 38.231 | −38.740 | 1.00 | 39.60 | W | O |
| ATOM | 7084 | N | GLU | W | 81 | 18.615 | 38.660 | −37.747 | 1.00 | 44.64 | W | N |
| ATOM | 7085 | CA | GLU | W | 81 | 19.111 | 38.582 | −36.376 | 1.00 | 49.96 | W | C |
| ATOM | 7086 | CB | GLU | W | 81 | 17.957 | 38.777 | −35.381 | 1.00 | 54.00 | W | C |
| ATOM | 7087 | CG | GLU | W | 81 | 17.138 | 40.058 | −35.573 | 1.00 | 67.47 | W | C |
| ATOM | 7088 | CD | GLU | W | 81 | 17.980 | 41.342 | −35.528 | 1.00 | 71.44 | W | C |
| ATOM | 7089 | OE1 | GLU | W | 81 | 18.845 | 41.481 | −34.628 | 1.00 | 74.08 | W | O |
| ATOM | 7090 | OE2 | GLU | W | 81 | 17.767 | 42.220 | −36.397 | 1.00 | 72.07 | W | O |
| ATOM | 7091 | C | GLU | W | 81 | 19.780 | 37.234 | −36.085 | 1.00 | 50.99 | W | C |
| ATOM | 7092 | O | GLU | W | 81 | 20.551 | 37.124 | −35.136 | 1.00 | 56.55 | W | O |
| ATOM | 7093 | N | ASP | W | 82 | 19.473 | 36.216 | −36.891 | 1.00 | 47.45 | W | N |
| ATOM | 7094 | CA | ASP | W | 82 | 20.029 | 34.872 | −36.713 | 1.00 | 41.26 | W | C |
| ATOM | 7095 | CB | ASP | W | 82 | 19.088 | 33.814 | −37.293 | 1.00 | 41.46 | W | C |
| ATOM | 7096 | CG | ASP | W | 82 | 17.743 | 33.819 | −36.638 | 1.00 | 43.98 | W | C |
| ATOM | 7097 | OD1 | ASP | W | 82 | 17.669 | 34.013 | −35.406 | 1.00 | 49.55 | W | O |
| ATOM | 7098 | OD2 | ASP | W | 82 | 16.749 | 33.641 | −37.361 | 1.00 | 47.22 | W | O |
| ATOM | 7099 | C | ASP | W | 82 | 21.428 | 34.640 | −37.278 | 1.00 | 38.70 | W | C |
| ATOM | 7100 | O | ASP | W | 82 | 21.968 | 33.536 | −37.150 | 1.00 | 33.51 | W | O |
| ATOM | 7101 | N | GLU | W | 83 | 21.999 | 35.637 | −37.952 | 1.00 | 38.43 | W | N |
| ATOM | 7102 | CA | GLU | W | 83 | 23.355 | 35.473 | −38.501 | 1.00 | 39.55 | W | C |
| ATOM | 7103 | CB | GLU | W | 83 | 23.742 | 36.645 | −39.422 | 1.00 | 43.87 | W | C |
| ATOM | 7104 | CG | GLU | W | 83 | 25.170 | 36.583 | −39.985 | 1.00 | 50.76 | W | C |
| ATOM | 7105 | CD | GLU | W | 83 | 25.348 | 37.330 | −41.323 | 1.00 | 59.64 | W | C |
| ATOM | 7106 | OE1 | GLU | W | 83 | 24.389 | 37.978 | −41.815 | 1.00 | 59.69 | W | O |
| ATOM | 7107 | OE2 | GLU | W | 83 | 26.461 | 37.249 | −41.902 | 1.00 | 64.93 | W | O |
| ATOM | 7108 | C | GLU | W | 83 | 24.308 | 35.374 | −37.314 | 1.00 | 36.66 | W | C |
| ATOM | 7109 | O | GLU | W | 83 | 24.307 | 36.233 | −36.429 | 1.00 | 36.91 | W | O |
| ATOM | 7110 | N | ALA | W | 84 | 25.061 | 34.282 | −37.269 | 1.00 | 35.43 | W | N |
| ATOM | 7111 | CA | ALA | W | 84 | 25.994 | 34.026 | −36.184 | 1.00 | 32.17 | W | C |
| ATOM | 7112 | CB | ALA | W | 84 | 25.230 | 33.896 | −34.874 | 1.00 | 35.92 | W | C |
| ATOM | 7113 | C | ALA | W | 84 | 26.745 | 32.739 | −36.443 | 1.00 | 32.40 | W | C |
| ATOM | 7114 | O | ALA | W | 84 | 26.599 | 32.129 | −37.508 | 1.00 | 31.87 | W | O |
| ATOM | 7115 | N | ILE | W | 85 | 27.569 | 32.351 | −35.468 | 1.00 | 30.86 | W | N |
| ATOM | 7116 | CA | ILE | W | 85 | 28.316 | 31.098 | −35.517 | 1.00 | 25.81 | W | C |
| ATOM | 7117 | CB | ILE | W | 85 | 29.762 | 31.273 | −35.067 | 1.00 | 27.16 | W | C |
| ATOM | 7118 | CG2 | ILE | W | 85 | 30.513 | 29.917 | −35.072 | 1.00 | 19.62 | W | C |
| ATOM | 7119 | CG1 | ILE | W | 85 | 30.455 | 32.253 | −36.003 | 1.00 | 30.19 | W | C |
| ATOM | 7120 | CD1 | ILE | W | 85 | 31.937 | 32.318 | −35.789 | 1.00 | 36.31 | W | C |
| ATOM | 7121 | C | ILE | W | 85 | 27.596 | 30.153 | −34.555 | 1.00 | 30.02 | W | C |
| ATOM | 7122 | O | ILE | W | 85 | 27.291 | 30.526 | −33.426 | 1.00 | 34.70 | W | O |
| ATOM | 7123 | N | TYR | W | 86 | 27.277 | 28.950 | −35.016 | 1.00 | 28.26 | W | N |
| ATOM | 7124 | CA | TYR | W | 86 | 26.572 | 28.002 | −34.175 | 1.00 | 25.60 | W | C |
| ATOM | 7125 | CB | TYR | W | 86 | 25.306 | 27.518 | −34.874 | 1.00 | 24.02 | W | C |
| ATOM | 7126 | CG | TYR | W | 86 | 24.266 | 28.602 | −34.999 | 1.00 | 25.92 | W | C |
| ATOM | 7127 | CD1 | TYR | W | 86 | 24.276 | 29.466 | −36.081 | 1.00 | 26.07 | W | C |
| ATOM | 7128 | CE1 | TYR | W | 86 | 23.372 | 30.520 | −36.177 | 1.00 | 26.86 | W | C |
| ATOM | 7129 | CD2 | TYR | W | 86 | 23.313 | 28.808 | −34.003 | 1.00 | 21.49 | W | C |
| ATOM | 7130 | CE2 | TYR | W | 86 | 22.400 | 29.870 | −34.092 | 1.00 | 23.13 | W | C |
| ATOM | 7131 | CZ | TYR | W | 86 | 22.444 | 30.718 | −35.186 | 1.00 | 23.63 | W | C |
| ATOM | 7132 | OH | TYR | W | 86 | 21.579 | 31.778 | −35.311 | 1.00 | 26.63 | W | O |
| ATOM | 7133 | C | TYR | W | 86 | 27.439 | 26.833 | −33.806 | 1.00 | 25.48 | W | C |
| ATOM | 7134 | O | TYR | W | 86 | 27.948 | 26.123 | −34.665 | 1.00 | 30.47 | W | O |
| ATOM | 7135 | N | PHE | W | 87 | 27.652 | 26.656 | −32.516 | 1.00 | 25.72 | W | N |
| ATOM | 7136 | CA | PHE | W | 87 | 28.461 | 25.539 | −32.062 | 1.00 | 23.75 | W | C |
| ATOM | 7137 | CB | PHE | W | 87 | 29.447 | 25.949 | −30.966 | 1.00 | 23.58 | W | C |
| ATOM | 7138 | CG | PHE | W | 87 | 30.609 | 26.757 | −31.461 | 1.00 | 23.44 | W | C |
| ATOM | 7139 | CD1 | PHE | W | 87 | 31.771 | 26.129 | −31.903 | 1.00 | 17.79 | W | C |
| ATOM | 7140 | CD2 | PHE | W | 87 | 30.544 | 28.147 | −31.475 | 1.00 | 18.25 | W | C |
| ATOM | 7141 | CE1 | PHE | W | 87 | 32.863 | 26.883 | −32.354 | 1.00 | 23.79 | W | C |
| ATOM | 7142 | CE2 | PHE | W | 87 | 31.637 | 28.915 | −31.929 | 1.00 | 26.35 | W | C |
| ATOM | 7143 | CZ | PHE | W | 87 | 32.792 | 28.285 | −32.366 | 1.00 | 18.19 | W | C |
| ATOM | 7144 | C | PHE | W | 87 | 27.571 | 24.456 | −31.516 | 1.00 | 25.85 | W | C |
| ATOM | 7145 | O | PHE | W | 87 | 26.537 | 24.725 | −30.886 | 1.00 | 20.62 | W | O |
| ATOM | 7146 | N | CYS | W | 88 | 28.007 | 23.232 | −31.774 | 1.00 | 25.40 | W | N |
| ATOM | 7147 | CA | CYS | W | 88 | 27.355 | 22.018 | −31.346 | 1.00 | 24.55 | W | C |
| ATOM | 7148 | C | CYS | W | 88 | 28.261 | 21.391 | −30.299 | 1.00 | 25.48 | W | C |
| ATOM | 7149 | O | CYS | W | 88 | 29.483 | 21.473 | −30.415 | 1.00 | 24.36 | W | O |
| ATOM | 7150 | CB | CYS | W | 88 | 27.279 | 21.046 | −32.518 | 1.00 | 24.05 | W | C |
| ATOM | 7151 | SG | CYS | W | 88 | 26.434 | 19.518 | −32.053 | 1.00 | 47.47 | W | S |
| ATOM | 7152 | N | ALA | W | 89 | 27.671 | 20.732 | −29.306 | 1.00 | 25.35 | W | N |
| ATOM | 7153 | CA | ALA | W | 89 | 28.442 | 20.039 | −28.277 | 1.00 | 17.70 | W | C |
| ATOM | 7154 | CB | ALA | W | 89 | 28.598 | 20.902 | −27.042 | 1.00 | 21.58 | W | C |
| ATOM | 7155 | C | ALA | W | 89 | 27.757 | 18.730 | −27.919 | 1.00 | 20.94 | W | C |
| ATOM | 7156 | O | ALA | W | 89 | 26.530 | 18.682 | −27.738 | 1.00 | 27.66 | W | O |
| ATOM | 7157 | N | LEU | W | 90 | 28.545 | 17.661 | −27.842 | 1.00 | 22.54 | W | N |
| ATOM | 7158 | CA | LEU | W | 90 | 28.023 | 16.337 | −27.492 | 1.00 | 24.00 | W | C |
| ATOM | 7159 | CB | LEU | W | 90 | 28.217 | 15.328 | −28.637 | 1.00 | 19.93 | W | C |
| ATOM | 7160 | CG | LEU | W | 90 | 27.546 | 15.674 | −29.981 | 1.00 | 22.25 | W | C |
| ATOM | 7161 | CD1 | LEU | W | 90 | 27.797 | 14.571 | −30.995 | 1.00 | 18.61 | W | C |
| ATOM | 7162 | CD2 | LEU | W | 90 | 26.066 | 15.876 | −29.798 | 1.00 | 18.36 | W | C |
| ATOM | 7163 | C | LEU | W | 90 | 28.706 | 15.833 | −26.235 | 1.00 | 26.12 | W | C |
| ATOM | 7164 | O | LEU | W | 90 | 29.891 | 16.076 | −26.021 | 1.00 | 30.58 | W | O |
| ATOM | 7165 | N | TRP | W | 91 | 27.931 | 15.143 | −25.408 | 1.00 | 26.27 | W | N |
| ATOM | 7166 | CA | TRP | W | 91 | 28.380 | 14.591 | −24.153 | 1.00 | 25.12 | W | C |
| ATOM | 7167 | CB | TRP | W | 91 | 27.282 | 14.809 | −23.116 | 1.00 | 25.63 | W | C |
| ATOM | 7168 | CG | TRP | W | 91 | 27.527 | 14.165 | −21.796 | 1.00 | 29.36 | W | C |
| ATOM | 7169 | CD2 | TRP | W | 91 | 28.348 | 14.664 | −20.737 | 1.00 | 29.80 | W | C |
| ATOM | 7170 | CE2 | TRP | W | 91 | 28.229 | 13.759 | −19.657 | 1.00 | 30.72 | W | C |
| ATOM | 7171 | CE3 | TRP | W | 91 | 29.163 | 15.788 | −20.589 | 1.00 | 30.94 | W | C |
| ATOM | 7172 | CD1 | TRP | W | 91 | 26.970 | 13.010 | −21.336 | 1.00 | 30.86 | W | C |
| ATOM | 7173 | NE1 | TRP | W | 91 | 27.387 | 12.755 | −20.055 | 1.00 | 27.96 | W | N |
| ATOM | 7174 | CZ2 | TRP | W | 91 | 28.900 | 13.948 | −18.438 | 1.00 | 22.68 | W | C |
| ATOM | 7175 | CZ3 | TRP | W | 91 | 29.832 | 15.975 | −19.375 | 1.00 | 32.71 | W | C |
| ATOM | 7176 | CH2 | TRP | W | 91 | 29.692 | 15.059 | −18.320 | 1.00 | 27.65 | W | C |
| ATOM | 7177 | C | TRP | W | 91 | 28.705 | 13.107 | −24.300 | 1.00 | 28.42 | W | C |
| ATOM | 7178 | O | TRP | W | 91 | 27.890 | 12.322 | −24.792 | 1.00 | 31.49 | W | O |
| ATOM | 7179 | N | TYR | W | 92 | 29.901 | 12.732 | −23.863 | 1.00 | 28.92 | W | N |
| ATOM | 7180 | CA | TYR | W | 92 | 30.370 | 11.354 | −23.926 | 1.00 | 33.44 | W | C |
| ATOM | 7181 | CB | TYR | W | 92 | 31.651 | 11.243 | −24.774 | 1.00 | 30.41 | W | C |
| ATOM | 7182 | CG | TYR | W | 92 | 31.453 | 11.572 | −26.234 | 1.00 | 32.25 | W | C |
| ATOM | 7183 | CD1 | TYR | W | 92 | 30.869 | 10.648 | −27.098 | 1.00 | 33.81 | W | C |
| ATOM | 7184 | CE1 | TYR | W | 92 | 30.630 | 10.957 | −28.426 | 1.00 | 33.34 | W | C |
| ATOM | 7185 | CD2 | TYR | W | 92 | 31.803 | 12.821 | −26.743 | 1.00 | 33.57 | W | C |
| ATOM | 7186 | CE2 | TYR | W | 92 | 31.565 | 13.143 | −28.078 | 1.00 | 36.39 | W | C |
| ATOM | 7187 | CZ | TYR | W | 92 | 30.974 | 12.201 | −28.908 | 1.00 | 38.06 | W | C |
| ATOM | 7188 | OH | TYR | W | 92 | 30.696 | 12.506 | −30.217 | 1.00 | 39.93 | W | O |
| ATOM | 7189 | C | TYR | W | 92 | 30.635 | 10.796 | −22.534 | 1.00 | 32.73 | W | C |
| ATOM | 7190 | O | TYR | W | 92 | 31.791 | 10.625 | −22.128 | 1.00 | 34.12 | W | O |
| ATOM | 7191 | N | SER | W | 93 | 29.554 | 10.558 | −21.796 | 1.00 | 31.30 | W | N |
| ATOM | 7192 | CA | SER | W | 93 | 29.611 | 9.990 | −20.446 | 1.00 | 32.90 | W | C |
| ATOM | 7193 | CB | SER | W | 93 | 30.367 | 8.652 | −20.473 | 1.00 | 35.61 | W | C |
| ATOM | 7194 | OG | SER | W | 93 | 29.786 | 7.757 | −21.420 | 1.00 | 42.23 | W | O |
| ATOM | 7195 | C | SER | W | 93 | 30.162 | 10.875 | −19.322 | 1.00 | 29.55 | W | C |
| ATOM | 7196 | O | SER | W | 93 | 29.506 | 11.053 | −18.302 | 1.00 | 29.52 | W | O |
| ATOM | 7197 | N | ASN | W | 94 | 31.327 | 11.475 | −19.538 | 1.00 | 27.57 | W | N |
| ATOM | 7198 | CA | ASN | W | 94 | 31.967 | 12.287 | −18.516 | 1.00 | 28.50 | W | C |
| ATOM | 7199 | CB | ASN | W | 94 | 33.039 | 11.451 | −17.841 | 1.00 | 20.69 | W | C |
| ATOM | 7200 | CG | ASN | W | 94 | 33.988 | 10.861 | −18.833 | 1.00 | 27.56 | W | C |
| ATOM | 7201 | OD1 | ASN | W | 94 | 34.190 | 11.425 | −19.907 | 1.00 | 32.71 | W | O |
| ATOM | 7202 | ND2 | ASN | W | 94 | 34.560 | 9.712 | −18.509 | 1.00 | 25.26 | W | N |
| ATOM | 7203 | C | ASN | W | 94 | 32.594 | 13.592 | −18.996 | 1.00 | 29.51 | W | C |
| ATOM | 7204 | O | ASN | W | 94 | 33.309 | 14.237 | −18.236 | 1.00 | 39.79 | W | O |
| ATOM | 7205 | N | HIS | W | 95 | 32.376 | 13.960 | −20.254 | 1.00 | 34.02 | W | N |
| ATOM | 7206 | CA | HIS | W | 95 | 32.930 | 15.209 | −20.790 | 1.00 | 27.54 | W | C |
| ATOM | 7207 | CB | HIS | W | 95 | 34.421 | 15.086 | −21.097 | 1.00 | 28.38 | W | C |
| ATOM | 7208 | CG | HIS | W | 95 | 34.738 | 14.133 | −22.210 | 1.00 | 30.83 | W | C |
| ATOM | 7209 | CD2 | HIS | W | 95 | 35.127 | 14.353 | −23.489 | 1.00 | 25.57 | W | C |
| ATOM | 7210 | ND1 | HIS | W | 95 | 34.683 | 12.764 | −22.057 | 1.00 | 31.10 | W | N |
| ATOM | 7211 | CE1 | HIS | W | 95 | 35.027 | 12.181 | −23.190 | 1.00 | 25.43 | W | C |
| ATOM | 7212 | NE2 | HIS | W | 95 | 35.301 | 13.123 | −24.075 | 1.00 | 30.14 | W | N |
| ATOM | 7213 | C | HIS | W | 95 | 32.231 | 15.657 | −22.045 | 1.00 | 28.98 | W | C |
| ATOM | 7214 | O | HIS | W | 95 | 31.524 | 14.882 | −22.704 | 1.00 | 28.22 | W | O |
| ATOM | 7215 | N | TRP | W | 96 | 32.411 | 16.929 | −22.357 | 1.00 | 23.72 | W | N |
| ATOM | 7216 | CA | TRP | W | 96 | 31.824 | 17.508 | −23.549 | 1.00 | 27.21 | W | C |
| ATOM | 7217 | CB | TRP | W | 96 | 31.256 | 18.891 | −23.268 | 1.00 | 22.09 | W | C |
| ATOM | 7218 | CG | TRP | W | 96 | 30.061 | 18.955 | −22.412 | 1.00 | 27.48 | W | C |
| ATOM | 7219 | CD2 | TRP | W | 96 | 28.699 | 18.833 | −22.834 | 1.00 | 26.53 | W | C |
| ATOM | 7220 | CE2 | TRP | W | 96 | 27.888 | 19.114 | −21.708 | 1.00 | 25.13 | W | C |
| ATOM | 7221 | CE3 | TRP | W | 96 | 28.084 | 18.520 | −24.055 | 1.00 | 24.16 | W | C |
| ATOM | 7222 | CD1 | TRP | W | 96 | 30.024 | 19.278 | −21.084 | 1.00 | 28.51 | W | C |
| ATOM | 7223 | NE1 | TRP | W | 96 | 28.724 | 19.387 | −20.659 | 1.00 | 27.41 | W | N |
| ATOM | 7224 | CZ2 | TRP | W | 96 | 26.490 | 19.093 | −21.765 | 1.00 | 22.94 | W | C |
| ATOM | 7225 | CZ3 | TRP | W | 96 | 26.704 | 18.501 | −24.114 | 1.00 | 23.03 | W | C |
| ATOM | 7226 | CH2 | TRP | W | 96 | 25.918 | 18.785 | −22.969 | 1.00 | 22.02 | W | C |
| ATOM | 7227 | C | TRP | W | 96 | 32.910 | 17.690 | −24.592 | 1.00 | 29.69 | W | C |
| ATOM | 7228 | O | TRP | W | 96 | 34.065 | 17.940 | −24.249 | 1.00 | 36.21 | W | O |
| ATOM | 7229 | N | VAL | W | 97 | 32.526 | 17.568 | −25.861 | 1.00 | 30.83 | W | N |
| ATOM | 7230 | CA | VAL | W | 97 | 33.429 | 17.785 | −26.990 | 1.00 | 24.77 | W | C |
| ATOM | 7231 | CB | VAL | W | 97 | 33.787 | 16.493 | −27.736 | 1.00 | 24.86 | W | C |
| ATOM | 7232 | CG1 | VAL | W | 97 | 34.530 | 16.837 | −29.021 | 1.00 | 26.64 | W | C |
| ATOM | 7233 | CG2 | VAL | W | 97 | 34.668 | 15.601 | −26.867 | 1.00 | 20.68 | W | C |
| ATOM | 7234 | C | VAL | W | 97 | 32.647 | 18.681 | −27.930 | 1.00 | 29.11 | W | C |
| ATOM | 7235 | O | VAL | W | 97 | 31.490 | 18.383 | −28.262 | 1.00 | 25.99 | W | O |
| ATOM | 7236 | N | PHE | W | 98 | 33.250 | 19.803 | −28.311 | 1.00 | 26.43 | W | N |
| ATOM | 7237 | CA | PHE | W | 98 | 32.593 | 20.743 | −29.206 | 1.00 | 22.15 | W | C |
| ATOM | 7238 | CB | PHE | W | 98 | 32.987 | 22.185 | −28.876 | 1.00 | 20.10 | W | C |
| ATOM | 7239 | CG | PHE | W | 98 | 32.271 | 22.762 | −27.689 | 1.00 | 22.43 | W | C |
| ATOM | 7240 | CD1 | PHE | W | 98 | 32.782 | 22.609 | −26.401 | 1.00 | 13.61 | W | C |
| ATOM | 7241 | CD2 | PHE | W | 98 | 31.103 | 23.493 | −27.865 | 1.00 | 17.24 | W | C |
| ATOM | 7242 | CE1 | PHE | W | 98 | 32.138 | 23.178 | −25.330 | 1.00 | 18.48 | W | C |
| ATOM | 7243 | CE2 | PHE | W | 98 | 30.450 | 24.068 | −26.788 | 1.00 | 17.64 | W | C |
| ATOM | 7244 | CZ | PHE | W | 98 | 30.967 | 23.912 | −25.518 | 1.00 | 21.07 | W | C |
| ATOM | 7245 | C | PHE | W | 98 | 32.911 | 20.499 | −30.665 | 1.00 | 27.04 | W | C |
| ATOM | 7246 | O | PHE | W | 98 | 33.961 | 19.944 | −31.007 | 1.00 | 27.19 | W | O |
| ATOM | 7247 | N | GLY | W | 99 | 31.983 | 20.900 | −31.524 | 1.00 | 22.52 | W | N |
| ATOM | 7248 | CA | GLY | W | 99 | 32.215 | 20.781 | −32.944 | 1.00 | 24.37 | W | C |
| ATOM | 7249 | C | GLY | W | 99 | 32.901 | 22.071 | −33.315 | 1.00 | 23.16 | W | C |
| ATOM | 7250 | O | GLY | W | 99 | 32.864 | 23.024 | −32.522 | 1.00 | 17.40 | W | O |
| ATOM | 7251 | N | GLY | W | 100 | 33.475 | 22.125 | −34.519 | 1.00 | 20.44 | W | N |
| ATOM | 7252 | CA | GLY | W | 100 | 34.154 | 23.331 | −34.980 | 1.00 | 18.87 | W | C |
| ATOM | 7253 | C | GLY | W | 100 | 33.290 | 24.564 | −35.198 | 1.00 | 20.17 | W | C |
| ATOM | 7254 | O | GLY | W | 100 | 33.817 | 25.651 | −35.432 | 1.00 | 30.43 | W | O |
| ATOM | 7255 | N | GLY | W | 101 | 31.968 | 24.419 | −35.105 | 1.00 | 22.16 | W | N |
| ATOM | 7256 | CA | GLY | W | 101 | 31.083 | 25.559 | −35.310 | 1.00 | 24.01 | W | C |
| ATOM | 7257 | C | GLY | W | 101 | 30.728 | 25.805 | −36.768 | 1.00 | 20.60 | W | C |
| ATOM | 7258 | O | GLY | W | 101 | 31.490 | 25.440 | −37.671 | 1.00 | 22.10 | W | O |
| ATOM | 7259 | N | THR | W | 102 | 29.579 | 26.439 | −36.997 | 1.00 | 22.18 | W | N |
| ATOM | 7260 | CA | THR | W | 102 | 29.132 | 26.736 | −38.349 | 1.00 | 21.79 | W | C |
| ATOM | 7261 | CB | THR | W | 102 | 27.879 | 25.903 | −38.723 | 1.00 | 27.30 | W | C |
| ATOM | 7262 | OG1 | THR | W | 102 | 28.187 | 24.502 | −38.627 | 1.00 | 22.49 | W | O |
| ATOM | 7263 | CG2 | THR | W | 102 | 27.416 | 26.238 | −40.167 | 1.00 | 23.19 | W | C |
| ATOM | 7264 | C | THR | W | 102 | 28.786 | 28.204 | −38.459 | 1.00 | 25.76 | W | C |
| ATOM | 7265 | O | THR | W | 102 | 27.937 | 28.694 | −37.725 | 1.00 | 27.40 | W | O |
| ATOM | 7266 | N | LYS | W | 103 | 29.457 | 28.926 | −39.351 | 1.00 | 28.58 | W | N |
| ATOM | 7267 | CA | LYS | W | 103 | 29.132 | 30.339 | −39.485 | 1.00 | 34.75 | W | C |
| ATOM | 7268 | CB | LYS | W | 103 | 30.309 | 31.159 | −40.038 | 1.00 | 36.63 | W | C |
| ATOM | 7269 | CG | LYS | W | 103 | 29.962 | 32.653 | −40.261 | 1.00 | 40.71 | W | C |
| ATOM | 7270 | CD | LYS | W | 103 | 31.081 | 33.389 | −41.026 | 1.00 | 44.52 | W | C |
| ATOM | 7271 | CE | LYS | W | 103 | 30.655 | 34.782 | −41.507 | 1.00 | 43.56 | W | C |
| ATOM | 7272 | NZ | LYS | W | 103 | 30.490 | 35.776 | −40.391 | 1.00 | 43.03 | W | N |
| ATOM | 7273 | C | LYS | W | 103 | 27.959 | 30.392 | −40.426 | 1.00 | 26.42 | W | C |
| ATOM | 7274 | O | LYS | W | 103 | 28.042 | 29.894 | −41.541 | 1.00 | 31.67 | W | O |
| ATOM | 7275 | N | LEU | W | 104 | 26.846 | 30.921 | −39.945 | 1.00 | 28.30 | W | N |
| ATOM | 7276 | CA | LEU | W | 104 | 25.653 | 31.021 | −40.766 | 1.00 | 24.71 | W | C |
| ATOM | 7277 | CB | LEU | W | 104 | 24.397 | 30.559 | −40.016 | 1.00 | 19.82 | W | C |
| ATOM | 7278 | CG | LEU | W | 104 | 23.102 | 30.737 | −40.835 | 1.00 | 26.05 | W | C |
| ATOM | 7279 | CD1 | LEU | W | 104 | 23.012 | 29.671 | −41.939 | 1.00 | 20.74 | W | C |
| ATOM | 7280 | CD2 | LEU | W | 104 | 21.883 | 30.660 | −39.933 | 1.00 | 26.55 | W | C |
| ATOM | 7281 | C | LEU | W | 104 | 25.466 | 32.446 | −41.199 | 1.00 | 25.82 | W | C |
| ATOM | 7282 | O | LEU | W | 104 | 25.437 | 33.359 | −40.375 | 1.00 | 29.37 | W | O |
| ATOM | 7283 | N | THR | W | 105 | 25.334 | 32.627 | −42.505 | 1.00 | 25.87 | W | N |
| ATOM | 7284 | CA | THR | W | 105 | 25.127 | 33.945 | −43.075 | 1.00 | 29.80 | W | C |
| ATOM | 7285 | CB | THR | W | 105 | 26.046 | 34.155 | −44.290 | 1.00 | 29.71 | W | C |
| ATOM | 7286 | OG1 | THR | W | 105 | 27.402 | 34.232 | −43.849 | 1.00 | 30.45 | W | O |
| ATOM | 7287 | CG2 | THR | W | 105 | 25.672 | 35.417 | −45.031 | 1.00 | 30.30 | W | C |
| ATOM | 7288 | C | THR | W | 105 | 23.684 | 34.109 | −43.530 | 1.00 | 24.01 | W | C |
| ATOM | 7289 | O | THR | W | 105 | 23.146 | 33.247 | −44.214 | 1.00 | 34.78 | W | O |
| ATOM | 7290 | N | VAL | W | 106 | 23.047 | 35.190 | −43.104 | 1.00 | 27.06 | W | N |
| ATOM | 7291 | CA | VAL | W | 106 | 21.690 | 35.474 | −43.515 | 1.00 | 29.37 | W | C |
| ATOM | 7292 | CB | VAL | W | 106 | 20.732 | 35.633 | −42.324 | 1.00 | 33.12 | W | C |
| ATOM | 7293 | CG1 | VAL | W | 106 | 19.319 | 35.932 | −42.837 | 1.00 | 23.37 | W | C |
| ATOM | 7294 | CG2 | VAL | W | 106 | 20.722 | 34.339 | −41.490 | 1.00 | 27.54 | W | C |
| ATOM | 7295 | C | VAL | W | 106 | 21.777 | 36.742 | −44.349 | 1.00 | 31.00 | W | C |
| ATOM | 7296 | O | VAL | W | 106 | 21.837 | 37.857 | −43.819 | 1.00 | 38.98 | W | O |
| ATOM | 7297 | N | LEU | W | 107 | 21.875 | 36.522 | −45.662 | 1.00 | 33.77 | W | N |
| ATOM | 7298 | CA | LEU | W | 107 | 21.997 | 37.558 | −46.695 | 1.00 | 33.23 | W | C |
| ATOM | 7299 | CB | LEU | W | 107 | 21.703 | 36.963 | −48.083 | 1.00 | 26.23 | W | C |
| ATOM | 7300 | CG | LEU | W | 107 | 22.832 | 36.412 | −48.970 | 1.00 | 37.43 | W | C |
| ATOM | 7301 | CD1 | LEU | W | 107 | 24.055 | 36.066 | −48.165 | 1.00 | 35.44 | W | C |
| ATOM | 7302 | CD2 | LEU | W | 107 | 22.357 | 35.204 | −49.781 | 1.00 | 28.71 | W | C |
| ATOM | 7303 | C | LEU | W | 107 | 21.172 | 38.824 | −46.488 | 1.00 | 31.73 | W | C |
| ATOM | 7304 | O | LEU | W | 107 | 19.943 | 38.796 | −46.540 | 1.00 | 40.63 | W | O |
| ATOM | 7305 | N | GLY | W | 108 | 21.874 | 39.930 | −46.258 | 1.00 | 35.34 | W | N |
| ATOM | 7306 | CA | GLY | W | 108 | 21.232 | 41.215 | −46.052 | 1.00 | 29.44 | W | C |
| ATOM | 7307 | C | GLY | W | 108 | 21.708 | 42.228 | −47.069 | 1.00 | 29.79 | W | C |
| ATOM | 7308 | O | GLY | W | 108 | 21.370 | 43.410 | −46.972 | 1.00 | 31.58 | W | O |
| ATOM | 7309 | N | GLN | W | 109 | 22.503 | 41.771 | −48.040 | 1.00 | 30.94 | W | N |
| ATOM | 7310 | CA | GLN | W | 109 | 23.017 | 42.635 | −49.102 | 1.00 | 27.18 | W | C |
| ATOM | 7311 | CB | GLN | W | 109 | 24.108 | 43.571 | −48.568 | 1.00 | 32.56 | W | C |
| ATOM | 7312 | CG | GLN | W | 109 | 25.392 | 42.892 | −48.119 | 1.00 | 30.09 | W | C |
| ATOM | 7313 | CD | GLN | W | 109 | 26.415 | 43.901 | −47.650 | 1.00 | 31.70 | W | C |
| ATOM | 7314 | OE1 | GLN | W | 109 | 26.905 | 44.706 | −48.440 | 1.00 | 34.79 | W | O |
| ATOM | 7315 | NE2 | GLN | W | 109 | 26.731 | 43.879 | −46.357 | 1.00 | 30.26 | W | N |
| ATOM | 7316 | C | GLN | W | 109 | 23.560 | 41.809 | −50.254 | 1.00 | 27.92 | W | C |
| ATOM | 7317 | O | GLN | W | 109 | 23.722 | 40.600 | −50.137 | 1.00 | 26.07 | W | O |
| ATOM | 7318 | N | PRO | W | 110 | 23.822 | 42.450 | −51.399 | 1.00 | 30.37 | W | N |
| ATOM | 7319 | CD | PRO | W | 110 | 23.550 | 43.863 | −51.729 | 1.00 | 31.79 | W | C |
| ATOM | 7320 | CA | PRO | W | 110 | 24.353 | 41.728 | −52.561 | 1.00 | 29.49 | W | C |
| ATOM | 7321 | CB | PRO | W | 110 | 24.474 | 42.831 | −53.620 | 1.00 | 30.21 | W | C |
| ATOM | 7322 | CG | PRO | W | 110 | 23.409 | 43.817 | −53.228 | 1.00 | 31.15 | W | C |
| ATOM | 7323 | C | PRO | W | 110 | 25.725 | 41.108 | −52.285 | 1.00 | 28.91 | W | C |
| ATOM | 7324 | O | PRO | W | 110 | 26.470 | 41.583 | −51.431 | 1.00 | 28.14 | W | O |
| ATOM | 7325 | N | LYS | W | 111 | 26.033 | 40.033 | −53.006 | 1.00 | 32.12 | W | N |
| ATOM | 7326 | CA | LYS | W | 111 | 27.312 | 39.353 | −52.593 | 1.00 | 28.90 | W | C |
| ATOM | 7327 | CB | LYS | W | 111 | 27.414 | 38.193 | −53.888 | 1.00 | 35.76 | W | C |
| ATOM | 7328 | CG | LYS | W | 111 | 26.422 | 37.059 | −53.733 | 1.00 | 39.51 | W | C |
| ATOM | 7329 | CD | LYS | W | 111 | 26.458 | 36.445 | −52.356 | 1.00 | 36.18 | W | C |
| ATOM | 7330 | CE | LYS | W | 111 | 25.579 | 35.212 | −52.307 | 1.00 | 39.20 | W | C |
| ATOM | 7331 | NZ | LYS | W | 111 | 26.058 | 34.182 | −53.277 | 1.00 | 40.09 | W | N |
| ATOM | 7332 | C | LYS | W | 111 | 28.376 | 40.370 | −53.280 | 1.00 | 33.38 | W | C |
| ATOM | 7333 | O | LYS | W | 111 | 28.125 | 41.291 | −54.061 | 1.00 | 31.58 | W | O |
| ATOM | 7334 | N | SER | W | 112 | 29.570 | 40.206 | −52.732 | 1.00 | 26.26 | W | N |
| ATOM | 7335 | CA | SER | W | 112 | 30.651 | 41.120 | −53.053 | 1.00 | 26.21 | W | C |
| ATOM | 7336 | CB | SER | W | 112 | 30.714 | 42.240 | −52.017 | 1.00 | 26.98 | W | C |
| ATOM | 7337 | OG | SER | W | 112 | 31.547 | 43.294 | −52.475 | 1.00 | 28.77 | W | O |
| ATOM | 7338 | C | SER | W | 112 | 31.973 | 40.370 | −53.099 | 1.00 | 23.92 | W | C |
| ATOM | 7339 | O | SER | W | 112 | 32.318 | 39.649 | −52.165 | 1.00 | 25.78 | W | O |
| ATOM | 7340 | N | SER | W | 113 | 32.653 | 40.477 | −54.239 | 1.00 | 23.68 | W | N |
| ATOM | 7341 | CA | SER | W | 113 | 33.944 | 39.825 | −54.430 | 1.00 | 27.94 | W | C |
| ATOM | 7342 | CB | SER | W | 113 | 34.286 | 39.745 | −55.921 | 1.00 | 21.69 | W | C |
| ATOM | 7343 | OG | SER | W | 113 | 33.244 | 39.072 | −56.620 | 1.00 | 29.80 | W | O |
| ATOM | 7344 | C | SER | W | 113 | 35.013 | 40.616 | −53.689 | 1.00 | 27.07 | W | C |
| ATOM | 7345 | O | SER | W | 113 | 34.874 | 41.836 | −53.493 | 1.00 | 26.67 | W | O |
| ATOM | 7346 | N | PRO | W | 114 | 36.078 | 39.930 | −53.250 | 1.00 | 25.98 | W | N |
| ATOM | 7347 | CD | PRO | W | 114 | 36.277 | 38.472 | −53.303 | 1.00 | 24.30 | W | C |
| ATOM | 7348 | CA | PRO | W | 114 | 37.173 | 40.582 | −52.525 | 1.00 | 25.05 | W | C |
| ATOM | 7349 | CB | PRO | W | 114 | 37.973 | 39.402 | −51.976 | 1.00 | 22.90 | W | C |
| ATOM | 7350 | CG | PRO | W | 114 | 37.766 | 38.339 | −53.009 | 1.00 | 23.14 | W | C |
| ATOM | 7351 | C | PRO | W | 114 | 38.054 | 41.449 | −53.389 | 1.00 | 22.39 | W | C |
| ATOM | 7352 | O | PRO | W | 114 | 38.335 | 41.103 | −54.534 | 1.00 | 37.10 | W | O |
| ATOM | 7353 | N | SER | W | 115 | 38.392 | 42.624 | −52.865 | 1.00 | 25.56 | W | N |
| ATOM | 7354 | CA | SER | W | 115 | 39.329 | 43.538 | −53.512 | 1.00 | 27.68 | W | C |
| ATOM | 7355 | CB | SER | W | 115 | 39.039 | 44.995 | −53.117 | 1.00 | 30.83 | W | C |
| ATOM | 7356 | OG | SER | W | 115 | 38.149 | 45.618 | −54.035 | 1.00 | 36.33 | W | O |
| ATOM | 7357 | C | SER | W | 115 | 40.647 | 43.052 | −52.886 | 1.00 | 25.55 | W | C |
| ATOM | 7358 | O | SER | W | 115 | 40.772 | 42.980 | −51.662 | 1.00 | 26.78 | W | O |
| ATOM | 7359 | N | VAL | W | 116 | 41.581 | 42.633 | −53.727 | 1.00 | 21.63 | W | N |
| ATOM | 7360 | CA | VAL | W | 116 | 42.842 | 42.096 | −53.269 | 1.00 | 16.98 | W | C |
| ATOM | 7361 | CB | VAL | W | 116 | 43.050 | 40.741 | −53.933 | 1.00 | 18.20 | W | C |
| ATOM | 7362 | CG1 | VAL | W | 116 | 44.287 | 40.096 | −53.438 | 1.00 | 21.00 | W | C |
| ATOM | 7363 | CG2 | VAL | W | 116 | 41.829 | 39.862 | −53.677 | 1.00 | 24.92 | W | C |
| ATOM | 7364 | C | VAL | W | 116 | 43.992 | 43.030 | −53.613 | 1.00 | 20.07 | W | C |
| ATOM | 7365 | O | VAL | W | 116 | 44.015 | 43.622 | −54.688 | 1.00 | 16.34 | W | O |
| ATOM | 7366 | N | ASN | W | 117 | 44.938 | 43.175 | −52.693 | 1.00 | 17.27 | W | N |
| ATOM | 7367 | CA | ASN | W | 117 | 46.106 | 44.040 | −52.903 | 1.00 | 23.20 | W | C |
| ATOM | 7368 | CB | ASN | W | 117 | 45.915 | 45.395 | −52.197 | 1.00 | 25.81 | W | C |
| ATOM | 7369 | CG | ASN | W | 117 | 44.488 | 45.897 | −52.279 | 1.00 | 36.66 | W | C |
| ATOM | 7370 | OD1 | ASN | W | 117 | 44.151 | 46.696 | −53.161 | 1.00 | 46.63 | W | O |
| ATOM | 7371 | ND2 | ASN | W | 117 | 43.621 | 45.398 | −51.385 | 1.00 | 41.61 | W | N |
| ATOM | 7372 | C | ASN | W | 117 | 47.299 | 43.318 | −52.290 | 1.00 | 20.64 | W | C |
| ATOM | 7373 | O | ASN | W | 117 | 47.270 | 42.956 | −51.113 | 1.00 | 15.36 | W | O |
| ATOM | 7374 | N | LEU | W | 118 | 48.314 | 43.066 | −53.107 | 1.00 | 18.77 | W | N |
| ATOM | 7375 | CA | LEU | W | 118 | 49.510 | 42.363 | −52.657 | 1.00 | 21.90 | W | C |
| ATOM | 7376 | CB | LEU | W | 118 | 49.804 | 41.228 | −53.623 | 1.00 | 12.10 | W | C |
| ATOM | 7377 | CG | LEU | W | 118 | 51.159 | 40.551 | −53.541 | 1.00 | 20.69 | W | C |
| ATOM | 7378 | CD1 | LEU | W | 118 | 51.332 | 39.896 | −52.188 | 1.00 | 18.78 | W | C |
| ATOM | 7379 | CD2 | LEU | W | 118 | 51.268 | 39.538 | −54.683 | 1.00 | 17.96 | W | C |
| ATOM | 7380 | C | LEU | W | 118 | 50.676 | 43.356 | −52.591 | 1.00 | 22.76 | W | C |
| ATOM | 7381 | O | LEU | W | 118 | 50.902 | 44.131 | −53.523 | 1.00 | 22.90 | W | O |
| ATOM | 7382 | N | PHE | W | 119 | 51.388 | 43.361 | −51.472 | 1.00 | 24.55 | W | N |
| ATOM | 7383 | CA | PHE | W | 119 | 52.509 | 44.285 | −51.298 | 1.00 | 20.33 | W | C |
| ATOM | 7384 | CB | PHE | W | 119 | 52.316 | 45.140 | −50.036 | 1.00 | 18.67 | W | C |
| ATOM | 7385 | CG | PHE | W | 119 | 51.205 | 46.163 | −50.128 | 1.00 | 13.38 | W | C |
| ATOM | 7386 | CD1 | PHE | W | 119 | 51.372 | 47.333 | −50.845 | 1.00 | 9.56 | W | C |
| ATOM | 7387 | CD2 | PHE | W | 119 | 50.033 | 46.003 | −49.398 | 1.00 | 19.18 | W | C |
| ATOM | 7388 | CE1 | PHE | W | 119 | 50.397 | 48.342 | −50.826 | 1.00 | 11.74 | W | C |
| ATOM | 7389 | CE2 | PHE | W | 119 | 49.047 | 47.010 | −49.377 | 1.00 | 16.83 | W | C |
| ATOM | 7390 | CZ | PHE | W | 119 | 49.237 | 48.181 | −50.088 | 1.00 | 11.72 | W | C |
| ATOM | 7391 | C | PHE | W | 119 | 53.853 | 43.572 | −51.214 | 1.00 | 15.76 | W | C |
| ATOM | 7392 | O | PHE | W | 119 | 53.965 | 42.485 | −50.634 | 1.00 | 19.26 | W | O |
| ATOM | 7393 | N | PRO | W | 120 | 54.869 | 44.136 | −51.877 | 1.00 | 17.03 | W | N |
| ATOM | 7394 | CD | PRO | W | 120 | 54.764 | 45.288 | −52.804 | 1.00 | 15.58 | W | C |
| ATOM | 7395 | CA | PRO | W | 120 | 56.222 | 43.566 | −51.876 | 1.00 | 20.20 | W | C |
| ATOM | 7396 | CB | PRO | W | 120 | 56.898 | 44.293 | −53.041 | 1.00 | 15.62 | W | C |
| ATOM | 7397 | CG | PRO | W | 120 | 56.214 | 45.670 | −53.009 | 1.00 | 17.75 | W | C |
| ATOM | 7398 | C | PRO | W | 120 | 56.873 | 43.999 | −50.576 | 1.00 | 22.76 | W | C |
| ATOM | 7399 | O | PRO | W | 120 | 56.341 | 44.859 | −49.887 | 1.00 | 18.01 | W | O |
| ATOM | 7400 | N | PRO | W | 121 | 58.015 | 43.399 | −50.212 | 1.00 | 25.09 | W | N |
| ATOM | 7401 | CD | PRO | W | 121 | 58.689 | 42.232 | −50.810 | 1.00 | 26.98 | W | C |
| ATOM | 7402 | CA | PRO | W | 121 | 58.681 | 43.804 | −48.972 | 1.00 | 23.77 | W | C |
| ATOM | 7403 | CB | PRO | W | 121 | 59.690 | 42.680 | −48.750 | 1.00 | 22.59 | W | C |
| ATOM | 7404 | CG | PRO | W | 121 | 60.034 | 42.262 | −50.140 | 1.00 | 29.46 | W | C |
| ATOM | 7405 | C | PRO | W | 121 | 59.375 | 45.147 | −49.204 | 1.00 | 22.35 | W | C |
| ATOM | 7406 | O | PRO | W | 121 | 59.806 | 45.448 | −50.321 | 1.00 | 22.73 | W | O |
| ATOM | 7407 | N | SER | W | 122 | 59.411 | 45.986 | −48.177 | 1.00 | 24.15 | W | N |
| ATOM | 7408 | CA | SER | W | 122 | 60.059 | 47.300 | −48.277 | 1.00 | 26.50 | W | C |
| ATOM | 7409 | CB | SER | W | 122 | 59.681 | 48.152 | −47.070 | 1.00 | 25.90 | W | C |
| ATOM | 7410 | OG | SER | W | 122 | 60.139 | 47.526 | −45.886 | 1.00 | 22.77 | W | O |
| ATOM | 7411 | C | SER | W | 122 | 61.578 | 47.131 | −48.270 | 1.00 | 25.08 | W | C |
| ATOM | 7412 | O | SER | W | 122 | 62.088 | 46.133 | −47.774 | 1.00 | 25.35 | W | O |
| ATOM | 7413 | N | SER | W | 123 | 62.303 | 48.103 | −48.809 | 1.00 | 24.63 | W | N |
| ATOM | 7414 | CA | SER | W | 123 | 63.758 | 48.018 | −48.780 | 1.00 | 27.18 | W | C |
| ATOM | 7415 | CB | SER | W | 123 | 64.388 | 49.115 | −49.632 | 1.00 | 23.07 | W | C |
| ATOM | 7416 | OG | SER | W | 123 | 63.893 | 50.374 | −49.243 | 1.00 | 39.90 | W | O |
| ATOM | 7417 | C | SER | W | 123 | 64.227 | 48.110 | −47.316 | 1.00 | 27.08 | W | C |
| ATOM | 7418 | O | SER | W | 123 | 65.217 | 47.486 | −46.933 | 1.00 | 28.48 | W | O |
| ATOM | 7419 | N | GLU | W | 124 | 63.473 | 48.822 | −46.481 | 1.00 | 23.95 | W | N |
| ATOM | 7420 | CA | GLU | W | 124 | 63.843 | 48.940 | −45.078 | 1.00 | 25.31 | W | C |
| ATOM | 7421 | CB | GLU | W | 124 | 62.923 | 49.900 | −44.321 | 1.00 | 29.47 | W | C |
| ATOM | 7422 | CG | GLU | W | 124 | 63.060 | 51.375 | −44.708 | 1.00 | 27.99 | W | C |
| ATOM | 7423 | CD | GLU | W | 124 | 62.167 | 51.791 | −45.881 | 1.00 | 36.41 | W | C |
| ATOM | 7424 | OE1 | GLU | W | 124 | 61.808 | 50.936 | −46.734 | 1.00 | 29.09 | W | O |
| ATOM | 7425 | OE2 | GLU | W | 124 | 61.825 | 52.995 | −45.950 | 1.00 | 40.70 | W | O |
| ATOM | 7426 | C | GLU | W | 124 | 63.854 | 47.570 | −44.407 | 1.00 | 26.47 | W | C |
| ATOM | 7427 | O | GLU | W | 124 | 64.838 | 47.198 | −43.767 | 1.00 | 27.90 | W | O |
| ATOM | 7428 | N | GLU | W | 125 | 62.782 | 46.800 | −44.572 | 1.00 | 24.14 | W | N |
| ATOM | 7429 | CA | GLU | W | 125 | 62.733 | 45.470 | −43.972 | 1.00 | 14.88 | W | C |
| ATOM | 7430 | CB | GLU | W | 125 | 61.349 | 44.849 | −44.069 | 1.00 | 24.33 | W | C |
| ATOM | 7431 | CG | GLU | W | 125 | 61.308 | 43.420 | −43.507 | 1.00 | 25.51 | W | C |
| ATOM | 7432 | CD | GLU | W | 125 | 60.031 | 42.666 | −43.833 | 1.00 | 28.63 | W | C |
| ATOM | 7433 | OE1 | GLU | W | 125 | 59.415 | 42.934 | −44.886 | 1.00 | 26.74 | W | O |
| ATOM | 7434 | OE2 | GLU | W | 125 | 59.649 | 41.785 | −43.034 | 1.00 | 31.91 | W | O |
| ATOM | 7435 | C | GLU | W | 125 | 63.757 | 44.503 | −44.564 | 1.00 | 20.99 | W | C |
| ATOM | 7436 | O | GLU | W | 125 | 64.252 | 43.633 | −43.845 | 1.00 | 20.43 | W | O |
| ATOM | 7437 | N | LEU | W | 126 | 64.082 | 44.636 | −45.855 | 1.00 | 18.80 | W | N |
| ATOM | 7438 | CA | LEU | W | 126 | 65.078 | 43.743 | −46.465 | 1.00 | 24.67 | W | C |
| ATOM | 7439 | CB | LEU | W | 126 | 65.188 | 43.944 | −47.986 | 1.00 | 18.33 | W | C |
| ATOM | 7440 | CG | LEU | W | 126 | 63.931 | 43.611 | −48.811 | 1.00 | 23.98 | W | C |
| ATOM | 7441 | CD1 | LEU | W | 126 | 64.078 | 44.141 | −50.246 | 1.00 | 19.81 | W | C |
| ATOM | 7442 | CD2 | LEU | W | 126 | 63.639 | 42.104 | −48.793 | 1.00 | 15.83 | W | C |
| ATOM | 7443 | C | LEU | W | 126 | 66.438 | 43.951 | −45.786 | 1.00 | 31.75 | W | C |
| ATOM | 7444 | O | LEU | W | 126 | 67.241 | 43.014 | −45.671 | 1.00 | 30.98 | W | O |
| ATOM | 7445 | N | LYS | W | 127 | 66.663 | 45.161 | −45.273 | 1.00 | 33.47 | W | N |
| ATOM | 7446 | CA | LYS | W | 127 | 67.898 | 45.447 | −44.565 | 1.00 | 33.03 | W | C |
| ATOM | 7447 | CB | LYS | W | 127 | 68.002 | 46.933 | −44.194 | 1.00 | 34.74 | W | C |
| ATOM | 7448 | CG | LYS | W | 127 | 68.611 | 47.802 | −45.305 | 1.00 | 36.15 | W | C |
| ATOM | 7449 | CD | LYS | W | 127 | 68.286 | 49.294 | −45.155 | 1.00 | 43.15 | W | C |
| ATOM | 7450 | CE | LYS | W | 127 | 68.735 | 49.852 | −43.804 | 1.00 | 48.29 | W | C |
| ATOM | 7451 | NZ | LYS | W | 127 | 68.620 | 51.354 | −43.659 | 1.00 | 47.45 | W | N |
| ATOM | 7452 | C | LYS | W | 127 | 68.030 | 44.542 | −43.339 | 1.00 | 32.64 | W | C |
| ATOM | 7453 | O | LYS | W | 127 | 69.149 | 44.279 | −42.905 | 1.00 | 38.78 | W | O |
| ATOM | 7454 | N | THR | W | 128 | 66.912 | 44.023 | −42.815 | 1.00 | 24.65 | W | N |
| ATOM | 7455 | CA | THR | W | 128 | 66.966 | 43.119 | −41.655 | 1.00 | 19.98 | W | C |
| ATOM | 7456 | CB | THR | W | 128 | 65.686 | 43.174 | −40.768 | 1.00 | 28.40 | W | C |
| ATOM | 7457 | OG1 | THR | W | 128 | 64.585 | 42.547 | −41.455 | 1.00 | 29.06 | W | O |
| ATOM | 7458 | CG2 | THR | W | 128 | 65.325 | 44.612 | −40.409 | 1.00 | 18.51 | W | C |
| ATOM | 7459 | C | THR | W | 128 | 67.134 | 41.673 | −42.114 | 1.00 | 26.35 | W | C |
| ATOM | 7460 | O | THR | W | 128 | 67.040 | 40.745 | −41.308 | 1.00 | 20.43 | W | O |
| ATOM | 7461 | N | LYS | W | 129 | 67.362 | 41.481 | −43.412 | 1.00 | 25.65 | W | N |
| ATOM | 7462 | CA | LYS | W | 129 | 67.533 | 40.142 | −43.979 | 1.00 | 35.31 | W | C |
| ATOM | 7463 | CB | LYS | W | 129 | 68.714 | 39.397 | −43.330 | 1.00 | 37.11 | W | C |
| ATOM | 7464 | CG | LYS | W | 129 | 70.079 | 39.993 | −43.695 | 1.00 | 46.18 | W | C |
| ATOM | 7465 | CD | LYS | W | 129 | 71.255 | 39.195 | −43.118 | 1.00 | 54.35 | W | C |
| ATOM | 7466 | CE | LYS | W | 129 | 71.489 | 37.849 | −43.829 | 1.00 | 55.81 | W | C |
| ATOM | 7467 | NZ | LYS | W | 129 | 72.625 | 37.069 | −43.219 | 1.00 | 51.88 | W | N |
| ATOM | 7468 | C | LYS | W | 129 | 66.262 | 39.290 | −43.929 | 1.00 | 38.43 | W | C |
| ATOM | 7469 | O | LYS | W | 129 | 66.315 | 38.049 | −43.968 | 1.00 | 38.86 | W | O |
| ATOM | 7470 | N | LYS | W | 130 | 65.120 | 39.972 | −43.853 | 1.00 | 37.43 | W | N |
| ATOM | 7471 | CA | LYS | W | 130 | 63.812 | 39.322 | −43.828 | 1.00 | 35.86 | W | C |
| ATOM | 7472 | CB | LYS | W | 130 | 63.200 | 39.346 | −42.417 | 1.00 | 38.86 | W | C |
| ATOM | 7473 | CG | LYS | W | 130 | 63.816 | 38.290 | −41.486 | 1.00 | 41.88 | W | C |
| ATOM | 7474 | CD | LYS | W | 130 | 63.408 | 38.490 | −40.026 | 1.00 | 51.02 | W | C |
| ATOM | 7475 | CE | LYS | W | 130 | 63.928 | 37.358 | −39.130 | 1.00 | 51.84 | W | C |
| ATOM | 7476 | NZ | LYS | W | 130 | 63.619 | 37.581 | −37.686 | 1.00 | 54.18 | W | N |
| ATOM | 7477 | C | LYS | W | 130 | 62.900 | 39.988 | −44.854 | 1.00 | 31.13 | W | C |
| ATOM | 7478 | O | LYS | W | 130 | 63.180 | 41.093 | −45.315 | 1.00 | 28.73 | W | O |
| ATOM | 7479 | N | ALA | W | 131 | 61.853 | 39.283 | −45.270 | 1.00 | 28.67 | W | N |
| ATOM | 7480 | CA | ALA | W | 131 | 60.932 | 39.828 | −46.257 | 1.00 | 24.40 | W | C |
| ATOM | 7481 | CB | ALA | W | 131 | 61.348 | 39.402 | −47.647 | 1.00 | 20.90 | W | C |
| ATOM | 7482 | C | ALA | W | 131 | 59.504 | 39.397 | −45.993 | 1.00 | 25.85 | W | C |
| ATOM | 7483 | O | ALA | W | 131 | 59.232 | 38.218 | −45.781 | 1.00 | 28.27 | W | O |
| ATOM | 7484 | N | THR | W | 132 | 58.589 | 40.357 | −46.001 | 1.00 | 22.49 | W | N |
| ATOM | 7485 | CA | THR | W | 132 | 57.192 | 40.037 | −45.779 | 1.00 | 25.69 | W | C |
| ATOM | 7486 | CB | THR | W | 132 | 56.665 | 40.609 | −44.456 | 1.00 | 23.79 | W | C |
| ATOM | 7487 | OG1 | THR | W | 132 | 57.454 | 40.120 | −43.373 | 1.00 | 29.56 | W | O |
| ATOM | 7488 | CG2 | THR | W | 132 | 55.241 | 40.166 | −44.240 | 1.00 | 27.41 | W | C |
| ATOM | 7489 | C | THR | W | 132 | 56.319 | 40.555 | −46.913 | 1.00 | 26.36 | W | C |
| ATOM | 7490 | O | THR | W | 132 | 56.406 | 41.722 | −47.301 | 1.00 | 25.70 | W | O |
| ATOM | 7491 | N | LEU | W | 133 | 55.510 | 39.666 | −47.473 | 1.00 | 21.06 | W | N |
| ATOM | 7492 | CA | LEU | W | 133 | 54.594 | 40.055 | −48.536 | 1.00 | 24.12 | W | C |
| ATOM | 7493 | CB | LEU | W | 133 | 54.489 | 38.946 | −49.593 | 1.00 | 28.36 | W | C |
| ATOM | 7494 | CG | LEU | W | 133 | 55.636 | 38.806 | −50.606 | 1.00 | 29.45 | W | C |
| ATOM | 7495 | CD1 | LEU | W | 133 | 55.885 | 37.352 | −51.021 | 1.00 | 28.56 | W | C |
| ATOM | 7496 | CD2 | LEU | W | 133 | 55.269 | 39.623 | −51.806 | 1.00 | 35.69 | W | C |
| ATOM | 7497 | C | LEU | W | 133 | 53.270 | 40.241 | −47.823 | 1.00 | 20.20 | W | C |
| ATOM | 7498 | O | LEU | W | 133 | 52.877 | 39.404 | −47.022 | 1.00 | 18.70 | W | O |
| ATOM | 7499 | N | VAL | W | 134 | 52.615 | 41.364 | −48.061 | 1.00 | 18.66 | W | N |
| ATOM | 7500 | CA | VAL | W | 134 | 51.338 | 41.627 | −47.415 | 1.00 | 20.25 | W | C |
| ATOM | 7501 | CB | VAL | W | 134 | 51.376 | 42.975 | −46.677 | 1.00 | 17.83 | W | C |
| ATOM | 7502 | CG1 | VAL | W | 134 | 50.069 | 43.233 | −45.967 | 1.00 | 24.16 | W | C |
| ATOM | 7503 | CG2 | VAL | W | 134 | 52.520 | 42.975 | −45.703 | 1.00 | 22.96 | W | C |
| ATOM | 7504 | C | VAL | W | 134 | 50.206 | 41.623 | −48.436 | 1.00 | 15.69 | W | C |
| ATOM | 7505 | O | VAL | W | 134 | 50.249 | 42.342 | −49.441 | 1.00 | 13.54 | W | O |
| ATOM | 7506 | N | CYS | W | 135 | 49.207 | 40.786 | −48.177 | 1.00 | 21.41 | W | N |
| ATOM | 7507 | CA | CYS | W | 135 | 48.035 | 40.664 | −49.048 | 1.00 | 20.16 | W | C |
| ATOM | 7508 | C | CYS | W | 135 | 46.793 | 41.149 | −48.310 | 1.00 | 18.93 | W | C |
| ATOM | 7509 | O | CYS | W | 135 | 46.325 | 40.481 | −47.384 | 1.00 | 19.27 | W | O |
| ATOM | 7510 | CB | CYS | W | 135 | 47.825 | 39.209 | −49.447 | 1.00 | 19.68 | W | C |
| ATOM | 7511 | SG | CYS | W | 135 | 46.465 | 38.953 | −50.637 | 1.00 | 27.88 | W | S |
| ATOM | 7512 | N | THR | W | 136 | 46.288 | 42.322 | −48.672 | 1.00 | 16.68 | W | N |
| ATOM | 7513 | CA | THR | W | 136 | 45.084 | 42.836 | −48.017 | 1.00 | 23.53 | W | C |
| ATOM | 7514 | CB | THR | W | 136 | 45.128 | 44.396 | −47.828 | 1.00 | 24.74 | W | C |
| ATOM | 7515 | OG1 | THR | W | 136 | 45.290 | 45.054 | −49.090 | 1.00 | 27.69 | W | O |
| ATOM | 7516 | CG2 | THR | W | 136 | 46.295 | 44.794 | −46.921 | 1.00 | 22.61 | W | C |
| ATOM | 7517 | C | THR | W | 136 | 43.850 | 42.389 | −48.811 | 1.00 | 23.26 | W | C |
| ATOM | 7518 | O | THR | W | 136 | 43.851 | 42.412 | −50.040 | 1.00 | 24.62 | W | O |
| ATOM | 7519 | N | ILE | W | 137 | 42.809 | 41.970 | −48.101 | 1.00 | 21.70 | W | N |
| ATOM | 7520 | CA | ILE | W | 137 | 41.575 | 41.478 | −48.719 | 1.00 | 16.54 | W | C |
| ATOM | 7521 | CB | ILE | W | 137 | 41.450 | 39.962 | −48.485 | 1.00 | 16.60 | W | C |
| ATOM | 7522 | CG2 | ILE | W | 137 | 40.264 | 39.384 | −49.271 | 1.00 | 9.26 | W | C |
| ATOM | 7523 | CG1 | ILE | W | 137 | 42.771 | 39.297 | −48.876 | 1.00 | 9.47 | W | C |
| ATOM | 7524 | CD1 | ILE | W | 137 | 42.966 | 37.941 | −48.340 | 1.00 | 12.04 | W | C |
| ATOM | 7525 | C | ILE | W | 137 | 40.389 | 42.182 | −48.073 | 1.00 | 21.48 | W | C |
| ATOM | 7526 | O | ILE | W | 137 | 40.068 | 41.928 | −46.909 | 1.00 | 22.81 | W | O |
| ATOM | 7527 | N | THR | W | 138 | 39.721 | 43.049 | −48.829 | 1.00 | 22.50 | W | N |
| ATOM | 7528 | CA | THR | W | 138 | 38.586 | 43.797 | −48.286 | 1.00 | 23.93 | W | C |
| ATOM | 7529 | CB | THR | W | 138 | 38.934 | 45.293 | −48.142 | 1.00 | 25.39 | W | C |
| ATOM | 7530 | OG1 | THR | W | 138 | 39.170 | 45.846 | −49.440 | 1.00 | 22.95 | W | O |
| ATOM | 7531 | CG2 | THR | W | 138 | 40.189 | 45.495 | −47.279 | 1.00 | 24.09 | W | C |
| ATOM | 7532 | C | THR | W | 138 | 37.304 | 43.733 | −49.121 | 1.00 | 29.16 | W | C |
| ATOM | 7533 | O | THR | W | 138 | 37.300 | 43.272 | −50.269 | 1.00 | 24.83 | W | O |
| ATOM | 7534 | N | GLU | W | 139 | 36.222 | 44.229 | −48.528 | 1.00 | 26.18 | W | N |
| ATOM | 7535 | CA | GLU | W | 139 | 34.932 | 44.298 | −49.188 | 1.00 | 29.04 | W | C |
| ATOM | 7536 | CB | GLU | W | 139 | 34.990 | 45.367 | −50.282 | 1.00 | 30.29 | W | C |
| ATOM | 7537 | CG | GLU | W | 139 | 34.732 | 46.776 | −49.779 | 1.00 | 40.60 | W | C |
| ATOM | 7538 | CD | GLU | W | 139 | 35.982 | 47.626 | −49.715 | 1.00 | 47.55 | W | C |
| ATOM | 7539 | OE1 | GLU | W | 139 | 36.904 | 47.401 | −50.535 | 1.00 | 54.18 | W | O |
| ATOM | 7540 | OE2 | GLU | W | 139 | 36.035 | 48.543 | −48.862 | 1.00 | 53.04 | W | O |
| ATOM | 7541 | C | GLU | W | 139 | 34.341 | 43.004 | −49.760 | 1.00 | 29.16 | W | C |
| ATOM | 7542 | O | GLU | W | 139 | 33.834 | 43.005 | −50.883 | 1.00 | 28.16 | W | O |
| ATOM | 7543 | N | PHE | W | 140 | 34.381 | 41.913 | −48.997 | 1.00 | 25.66 | W | N |
| ATOM | 7544 | CA | PHE | W | 140 | 33.838 | 40.655 | −49.481 | 1.00 | 25.73 | W | C |
| ATOM | 7545 | CB | PHE | W | 140 | 34.910 | 39.560 | −49.636 | 1.00 | 24.08 | W | C |
| ATOM | 7546 | CG | PHE | W | 140 | 35.616 | 39.162 | −48.352 | 1.00 | 25.09 | W | C |
| ATOM | 7547 | CD1 | PHE | W | 140 | 36.836 | 39.740 | −48.004 | 1.00 | 26.97 | W | C |
| ATOM | 7548 | CD2 | PHE | W | 140 | 35.113 | 38.151 | −47.546 | 1.00 | 24.94 | W | C |
| ATOM | 7549 | CE1 | PHE | W | 140 | 37.538 | 39.312 | −46.884 | 1.00 | 20.48 | W | C |
| ATOM | 7550 | CE2 | PHE | W | 140 | 35.812 | 37.718 | −46.422 | 1.00 | 23.22 | W | C |
| ATOM | 7551 | CZ | PHE | W | 140 | 37.023 | 38.299 | −46.095 | 1.00 | 27.21 | W | C |
| ATOM | 7552 | C | PHE | W | 140 | 32.657 | 40.185 | −48.662 | 1.00 | 33.16 | W | C |
| ATOM | 7553 | O | PHE | W | 140 | 32.636 | 40.331 | −47.436 | 1.00 | 35.55 | W | O |
| ATOM | 7554 | N | TYR | W | 141 | 31.666 | 39.626 | −49.356 | 1.00 | 33.01 | W | N |
| ATOM | 7555 | CA | TYR | W | 141 | 30.448 | 39.172 | −48.703 | 1.00 | 30.41 | W | C |
| ATOM | 7556 | CB | TYR | W | 141 | 29.508 | 40.380 | −48.538 | 1.00 | 25.31 | W | C |
| ATOM | 7557 | CG | TYR | W | 141 | 28.274 | 40.056 | −47.763 | 1.00 | 33.28 | W | C |
| ATOM | 7558 | CD1 | TYR | W | 141 | 28.323 | 39.922 | −46.375 | 1.00 | 29.11 | W | C |
| ATOM | 7559 | CE1 | TYR | W | 141 | 27.218 | 39.505 | −45.664 | 1.00 | 31.17 | W | C |
| ATOM | 7560 | CD2 | TYR | W | 141 | 27.071 | 39.773 | −48.420 | 1.00 | 29.26 | W | C |
| ATOM | 7561 | CE2 | TYR | W | 141 | 25.956 | 39.352 | −47.710 | 1.00 | 29.34 | W | C |
| ATOM | 7562 | CZ | TYR | W | 141 | 26.038 | 39.216 | −46.338 | 1.00 | 29.27 | W | C |
| ATOM | 7563 | OH | TYR | W | 141 | 24.960 | 38.750 | −45.633 | 1.00 | 35.92 | W | O |
| ATOM | 7564 | C | TYR | W | 141 | 29.754 | 38.040 | −49.470 | 1.00 | 24.67 | W | C |
| ATOM | 7565 | O | TYR | W | 141 | 29.553 | 38.128 | −50.679 | 1.00 | 29.80 | W | O |
| ATOM | 7566 | N | PRO | W | 142 | 29.390 | 36.955 | −48.779 | 1.00 | 26.28 | W | N |
| ATOM | 7567 | CD | PRO | W | 142 | 28.621 | 35.878 | −49.436 | 1.00 | 26.84 | W | C |
| ATOM | 7568 | CA | PRO | W | 142 | 29.555 | 36.681 | −47.346 | 1.00 | 26.58 | W | C |
| ATOM | 7569 | CB | PRO | W | 142 | 28.725 | 35.404 | −47.150 | 1.00 | 24.72 | W | C |
| ATOM | 7570 | CG | PRO | W | 142 | 28.801 | 34.722 | −48.479 | 1.00 | 27.26 | W | C |
| ATOM | 7571 | C | PRO | W | 142 | 31.026 | 36.538 | −46.895 | 1.00 | 27.87 | W | C |
| ATOM | 7572 | O | PRO | W | 142 | 31.925 | 36.355 | −47.723 | 1.00 | 35.63 | W | O |
| ATOM | 7573 | N | GLY | W | 143 | 31.253 | 36.634 | −45.584 | 1.00 | 31.25 | W | N |
| ATOM | 7574 | CA | GLY | W | 143 | 32.597 | 36.587 | −45.012 | 1.00 | 24.43 | W | C |
| ATOM | 7575 | C | GLY | W | 143 | 33.339 | 35.280 | −44.896 | 1.00 | 22.15 | W | C |
| ATOM | 7576 | O | GLY | W | 143 | 33.673 | 34.851 | −43.796 | 1.00 | 32.11 | W | O |
| ATOM | 7577 | N | ALA | W | 144 | 33.629 | 34.667 | −46.036 | 1.00 | 28.28 | W | N |
| ATOM | 7578 | CA | ALA | W | 144 | 34.353 | 33.398 | −46.098 | 1.00 | 29.26 | W | C |
| ATOM | 7579 | CB | ALA | W | 144 | 33.369 | 32.185 | −46.099 | 1.00 | 23.74 | W | C |
| ATOM | 7580 | C | ALA | W | 144 | 35.223 | 33.377 | −47.360 | 1.00 | 31.91 | W | C |
| ATOM | 7581 | O | ALA | W | 144 | 34.731 | 33.594 | −48.480 | 1.00 | 27.81 | W | O |
| ATOM | 7582 | N | VAL | W | 145 | 36.522 | 33.158 | −47.169 | 1.00 | 28.01 | W | N |
| ATOM | 7583 | CA | VAL | W | 145 | 37.468 | 33.084 | −48.276 | 1.00 | 18.88 | W | C |
| ATOM | 7584 | CB | VAL | W | 145 | 38.138 | 34.453 | −48.587 | 1.00 | 19.48 | W | C |
| ATOM | 7585 | CG1 | VAL | W | 145 | 37.158 | 35.447 | −49.198 | 1.00 | 25.12 | W | C |
| ATOM | 7586 | CG2 | VAL | W | 145 | 38.759 | 35.014 | −47.347 | 1.00 | 20.96 | W | C |
| ATOM | 7587 | C | VAL | W | 145 | 38.606 | 32.141 | −47.906 | 1.00 | 25.61 | W | C |
| ATOM | 7588 | O | VAL | W | 145 | 38.835 | 31.825 | −46.728 | 1.00 | 19.53 | W | O |
| ATOM | 7589 | N | ARG | W | 146 | 39.324 | 31.697 | −48.923 | 1.00 | 16.84 | W | N |
| ATOM | 7590 | CA | ARG | W | 146 | 40.482 | 30.857 | −48.710 | 1.00 | 27.23 | W | C |
| ATOM | 7591 | CB | ARG | W | 146 | 40.244 | 29.442 | −49.235 | 1.00 | 27.53 | W | C |
| ATOM | 7592 | CG | ARG | W | 146 | 38.962 | 28.828 | −48.700 | 1.00 | 33.16 | W | C |
| ATOM | 7593 | CD | ARG | W | 146 | 38.948 | 27.316 | −48.873 | 0.00 | 31.85 | W | C |
| ATOM | 7594 | NE | ARG | W | 146 | 39.650 | 26.634 | −47.786 | 0.00 | 32.95 | W | N |
| ATOM | 7595 | CZ | ARG | W | 146 | 40.682 | 25.810 | −47.947 | 0.00 | 32.86 | W | C |
| ATOM | 7596 | NH1 | ARG | W | 146 | 41.154 | 25.552 | −49.160 | 0.00 | 33.17 | W | N |
| ATOM | 7597 | NH2 | ARG | W | 146 | 41.238 | 25.235 | −46.890 | 0.00 | 32.67 | W | N |
| ATOM | 7598 | C | ARG | W | 146 | 41.620 | 31.564 | −49.462 | 1.00 | 28.95 | W | C |
| ATOM | 7599 | O | ARG | W | 146 | 41.424 | 32.083 | −50.571 | 1.00 | 28.44 | W | O |
| ATOM | 7600 | N | VAL | W | 147 | 42.786 | 31.659 | −48.828 | 1.00 | 27.00 | W | N |
| ATOM | 7601 | CA | VAL | W | 147 | 43.926 | 32.325 | −49.448 | 1.00 | 21.06 | W | C |
| ATOM | 7602 | CB | VAL | W | 147 | 44.476 | 33.443 | −48.529 | 1.00 | 22.60 | W | C |
| ATOM | 7603 | CG1 | VAL | W | 147 | 45.650 | 34.136 | −49.177 | 1.00 | 20.90 | W | C |
| ATOM | 7604 | CG2 | VAL | W | 147 | 43.393 | 34.449 | −48.235 | 1.00 | 20.73 | W | C |
| ATOM | 7605 | C | VAL | W | 147 | 45.036 | 31.343 | −49.787 | 1.00 | 19.97 | W | C |
| ATOM | 7606 | O | VAL | W | 147 | 45.307 | 30.423 | −49.041 | 1.00 | 25.23 | W | O |
| ATOM | 7607 | N | ASP | W | 148 | 45.665 | 31.527 | −50.931 | 1.00 | 19.35 | W | N |
| ATOM | 7608 | CA | ASP | W | 148 | 46.758 | 30.656 | −51.330 | 1.00 | 24.21 | W | C |
| ATOM | 7609 | CB | ASP | W | 148 | 46.295 | 29.584 | −52.330 | 1.00 | 27.78 | W | C |
| ATOM | 7610 | CG | ASP | W | 148 | 45.258 | 28.630 | −51.741 | 1.00 | 36.31 | W | C |
| ATOM | 7611 | OD1 | ASP | W | 148 | 45.591 | 27.864 | −50.802 | 1.00 | 36.59 | W | O |
| ATOM | 7612 | OD2 | ASP | W | 148 | 44.107 | 28.639 | −52.235 | 1.00 | 32.97 | W | O |
| ATOM | 7613 | C | ASP | W | 148 | 47.844 | 31.523 | −51.962 | 1.00 | 26.46 | W | C |
| ATOM | 7614 | O | ASP | W | 148 | 47.556 | 32.563 | −52.561 | 1.00 | 16.85 | W | O |
| ATOM | 7615 | N | TRP | W | 149 | 49.095 | 31.089 | −51.827 | 1.00 | 24.78 | W | N |
| ATOM | 7616 | CA | TRP | W | 149 | 50.222 | 31.829 | −52.374 | 1.00 | 21.82 | W | C |
| ATOM | 7617 | CB | TRP | W | 149 | 51.173 | 32.222 | −51.257 | 1.00 | 15.61 | W | C |
| ATOM | 7618 | CG | TRP | W | 149 | 50.640 | 33.275 | −50.347 | 1.00 | 15.46 | W | C |
| ATOM | 7619 | CD2 | TRP | W | 149 | 50.945 | 34.674 | −50.390 | 1.00 | 16.75 | W | C |
| ATOM | 7620 | CE2 | TRP | W | 149 | 50.252 | 35.283 | −49.326 | 1.00 | 15.22 | W | C |
| ATOM | 7621 | CE3 | TRP | W | 149 | 51.739 | 35.473 | −51.229 | 1.00 | 10.00 | W | C |
| ATOM | 7622 | CD1 | TRP | W | 149 | 49.802 | 33.098 | −49.289 | 1.00 | 10.87 | W | C |
| ATOM | 7623 | NE1 | TRP | W | 149 | 49.564 | 34.298 | −48.669 | 1.00 | 13.24 | W | N |
| ATOM | 7624 | CZ2 | TRP | W | 149 | 50.324 | 36.655 | −49.078 | 1.00 | 18.86 | W | C |
| ATOM | 7625 | CZ3 | TRP | W | 149 | 51.808 | 36.816 | −50.991 | 1.00 | 12.42 | W | C |
| ATOM | 7626 | CH2 | TRP | W | 149 | 51.103 | 37.407 | −49.919 | 1.00 | 16.44 | W | C |
| ATOM | 7627 | C | TRP | W | 149 | 50.974 | 31.015 | −53.406 | 1.00 | 20.74 | W | C |
| ATOM | 7628 | O | TRP | W | 149 | 50.965 | 29.793 | −53.363 | 1.00 | 27.94 | W | O |
| ATOM | 7629 | N | LYS | W | 150 | 51.593 | 31.706 | −54.352 | 1.00 | 23.20 | W | N |
| ATOM | 7630 | CA | LYS | W | 150 | 52.388 | 31.060 | −55.386 | 1.00 | 24.58 | W | C |
| ATOM | 7631 | CB | LYS | W | 150 | 51.660 | 31.044 | −56.733 | 1.00 | 29.86 | W | C |
| ATOM | 7632 | CG | LYS | W | 150 | 50.377 | 30.237 | −56.790 | 1.00 | 33.39 | W | C |
| ATOM | 7633 | CD | LYS | W | 150 | 49.875 | 30.121 | −58.237 | 1.00 | 39.19 | W | C |
| ATOM | 7634 | CE | LYS | W | 150 | 49.636 | 31.483 | −58.890 | 1.00 | 41.74 | W | C |
| ATOM | 7635 | NZ | LYS | W | 150 | 48.975 | 31.370 | −60.226 | 1.00 | 40.02 | W | N |
| ATOM | 7636 | C | LYS | W | 150 | 53.685 | 31.831 | −55.570 | 1.00 | 26.05 | W | C |
| ATOM | 7637 | O | LYS | W | 150 | 53.713 | 33.057 | −55.459 | 1.00 | 22.83 | W | O |
| ATOM | 7638 | N | ALA | W | 151 | 54.757 | 31.106 | −55.861 | 1.00 | 27.77 | W | N |
| ATOM | 7639 | CA | ALA | W | 151 | 56.047 | 31.726 | −56.113 | 1.00 | 30.66 | W | C |
| ATOM | 7640 | CB | ALA | W | 151 | 57.031 | 31.423 | −54.971 | 1.00 | 32.09 | W | C |
| ATOM | 7641 | C | ALA | W | 151 | 56.513 | 31.122 | −57.427 | 1.00 | 27.01 | W | C |
| ATOM | 7642 | O | ALA | W | 151 | 56.664 | 29.911 | −57.531 | 1.00 | 32.02 | W | O |
| ATOM | 7643 | N | ASP | W | 152 | 56.618 | 31.964 | −58.449 | 1.00 | 31.93 | W | N |
| ATOM | 7644 | CA | ASP | W | 152 | 57.051 | 31.556 | −59.790 | 1.00 | 38.33 | W | C |
| ATOM | 7645 | CB | ASP | W | 152 | 58.551 | 31.240 | −59.809 | 1.00 | 36.37 | W | C |
| ATOM | 7646 | CG | ASP | W | 152 | 59.415 | 32.472 | −59.628 | 1.00 | 35.72 | W | C |
| ATOM | 7647 | OD1 | ASP | W | 152 | 59.038 | 33.559 | −60.104 | 1.00 | 37.39 | W | O |
| ATOM | 7648 | OD2 | ASP | W | 152 | 60.506 | 32.347 | −59.044 | 1.00 | 40.58 | W | O |
| ATOM | 7649 | C | ASP | W | 152 | 56.257 | 30.394 | −60.392 | 1.00 | 40.25 | W | C |
| ATOM | 7650 | O | ASP | W | 152 | 56.816 | 29.526 | −61.059 | 1.00 | 41.77 | W | O |
| ATOM | 7651 | N | GLY | W | 153 | 54.954 | 30.373 | −60.139 | 1.00 | 40.50 | W | N |
| ATOM | 7652 | CA | GLY | W | 153 | 54.128 | 29.324 | −60.695 | 1.00 | 36.62 | W | C |
| ATOM | 7653 | C | GLY | W | 153 | 53.917 | 28.097 | −59.841 | 1.00 | 41.03 | W | C |
| ATOM | 7654 | O | GLY | W | 153 | 53.132 | 27.235 | −60.225 | 1.00 | 52.32 | W | O |
| ATOM | 7655 | N | THR | W | 154 | 54.596 | 27.981 | −58.704 | 1.00 | 40.84 | W | N |
| ATOM | 7656 | CA | THR | W | 154 | 54.399 | 26.800 | −57.860 | 1.00 | 33.05 | W | C |
| ATOM | 7657 | CB | THR | W | 154 | 55.704 | 25.995 | −57.610 | 1.00 | 41.99 | W | C |
| ATOM | 7658 | OG1 | THR | W | 154 | 56.331 | 26.440 | −56.395 | 1.00 | 41.29 | W | O |
| ATOM | 7659 | CG2 | THR | W | 154 | 56.665 | 26.138 | −58.791 | 1.00 | 31.78 | W | C |
| ATOM | 7660 | C | THR | W | 154 | 53.809 | 27.213 | −56.536 | 1.00 | 33.72 | W | C |
| ATOM | 7661 | O | THR | W | 154 | 54.104 | 28.284 | −56.027 | 1.00 | 37.29 | W | O |
| ATOM | 7662 | N | PRO | W | 155 | 52.977 | 26.349 | −55.950 | 1.00 | 34.88 | W | N |
| ATOM | 7663 | CD | PRO | W | 155 | 52.711 | 24.987 | −56.444 | 1.00 | 36.89 | W | C |
| ATOM | 7664 | CA | PRO | W | 155 | 52.303 | 26.578 | −54.667 | 1.00 | 35.46 | W | C |
| ATOM | 7665 | CB | PRO | W | 155 | 51.449 | 25.312 | −54.491 | 1.00 | 33.30 | W | C |
| ATOM | 7666 | CG | PRO | W | 155 | 51.378 | 24.700 | −55.847 | 1.00 | 33.90 | W | C |
| ATOM | 7667 | C | PRO | W | 155 | 53.275 | 26.665 | −53.504 | 1.00 | 38.95 | W | C |
| ATOM | 7668 | O | PRO | W | 155 | 54.327 | 26.010 | −53.523 | 1.00 | 42.27 | W | O |
| ATOM | 7669 | N | VAL | W | 156 | 52.958 | 27.498 | −52.513 | 1.00 | 33.35 | W | N |
| ATOM | 7670 | CA | VAL | W | 156 | 53.808 | 27.559 | −51.333 | 1.00 | 32.17 | W | C |
| ATOM | 7671 | CB | VAL | W | 156 | 54.348 | 29.007 | −50.957 | 1.00 | 32.55 | W | C |
| ATOM | 7672 | CG1 | VAL | W | 156 | 54.187 | 29.989 | −52.122 | 1.00 | 23.21 | W | C |
| ATOM | 7673 | CG2 | VAL | W | 156 | 53.748 | 29.519 | −49.655 | 1.00 | 24.33 | W | C |
| ATOM | 7674 | C | VAL | W | 156 | 52.962 | 26.921 | −50.246 | 1.00 | 35.17 | W | C |
| ATOM | 7675 | O | VAL | W | 156 | 51.755 | 27.163 | −50.162 | 1.00 | 33.64 | W | O |
| ATOM | 7676 | N | THR | W | 157 | 53.597 | 26.061 | −49.460 | 1.00 | 33.10 | W | N |
| ATOM | 7677 | CA | THR | W | 157 | 52.920 | 25.329 | −48.412 | 1.00 | 39.40 | W | C |
| ATOM | 7678 | CB | THR | W | 157 | 53.101 | 23.819 | −48.682 | 1.00 | 40.72 | W | C |
| ATOM | 7679 | OG1 | THR | W | 157 | 54.472 | 23.563 | −49.001 | 1.00 | 39.84 | W | O |
| ATOM | 7680 | CG2 | THR | W | 157 | 52.267 | 23.385 | −49.884 | 1.00 | 37.45 | W | C |
| ATOM | 7681 | C | THR | W | 157 | 53.370 | 25.709 | −46.993 | 1.00 | 44.10 | W | C |
| ATOM | 7682 | O | THR | W | 157 | 52.740 | 25.316 | −46.003 | 1.00 | 50.44 | W | O |
| ATOM | 7683 | N | GLN | W | 158 | 54.428 | 26.514 | −46.906 | 1.00 | 45.43 | W | N |
| ATOM | 7684 | CA | GLN | W | 158 | 54.992 | 26.964 | −45.629 | 1.00 | 46.33 | W | C |
| ATOM | 7685 | CB | GLN | W | 158 | 56.326 | 26.237 | −45.365 | 1.00 | 50.42 | W | C |
| ATOM | 7686 | CG | GLN | W | 158 | 56.225 | 24.846 | −44.727 | 1.00 | 59.51 | W | C |
| ATOM | 7687 | CD | GLN | W | 158 | 56.022 | 24.891 | −43.208 | 1.00 | 64.69 | W | C |
| ATOM | 7688 | OE1 | GLN | W | 158 | 55.747 | 25.951 | −42.626 | 1.00 | 65.30 | W | O |
| ATOM | 7689 | NE2 | GLN | W | 158 | 56.156 | 23.734 | −42.562 | 1.00 | 61.20 | W | N |
| ATOM | 7690 | C | GLN | W | 158 | 55.266 | 28.473 | −45.650 | 1.00 | 41.34 | W | C |
| ATOM | 7691 | O | GLN | W | 158 | 55.498 | 29.054 | −46.702 | 1.00 | 43.23 | W | O |
| ATOM | 7692 | N | GLY | W | 159 | 55.274 | 29.101 | −44.484 | 1.00 | 37.81 | W | N |
| ATOM | 7693 | CA | GLY | W | 159 | 55.565 | 30.523 | −44.426 | 1.00 | 34.13 | W | C |
| ATOM | 7694 | C | GLY | W | 159 | 54.414 | 31.490 | −44.619 | 1.00 | 34.62 | W | C |
| ATOM | 7695 | O | GLY | W | 159 | 54.634 | 32.697 | −44.650 | 1.00 | 35.40 | W | O |
| ATOM | 7696 | N | ASP | W | 160 | 53.197 | 30.968 | −44.768 | 1.00 | 38.90 | W | N |
| ATOM | 7697 | CA | ASP | W | 160 | 51.998 | 31.793 | −44.951 | 1.00 | 35.65 | W | C |
| ATOM | 7698 | CB | ASP | W | 160 | 51.139 | 31.279 | −46.132 | 1.00 | 37.64 | W | C |
| ATOM | 7699 | CG | ASP | W | 160 | 51.177 | 29.735 | −46.296 | 1.00 | 48.33 | W | C |
| ATOM | 7700 | OD1 | ASP | W | 160 | 51.214 | 28.987 | −45.291 | 1.00 | 54.91 | W | O |
| ATOM | 7701 | OD2 | ASP | W | 160 | 51.175 | 29.256 | −47.447 | 1.00 | 48.41 | W | O |
| ATOM | 7702 | C | ASP | W | 160 | 51.166 | 31.831 | −43.676 | 1.00 | 35.71 | W | C |
| ATOM | 7703 | O | ASP | W | 160 | 51.196 | 30.898 | −42.882 | 1.00 | 37.42 | W | O |
| ATOM | 7704 | N | GLU | W | 161 | 50.465 | 32.939 | −43.458 | 1.00 | 40.77 | W | N |
| ATOM | 7705 | CA | GLU | W | 161 | 49.586 | 33.084 | −42.300 | 1.00 | 39.71 | W | C |
| ATOM | 7706 | CB | GLU | W | 161 | 50.358 | 33.552 | −41.063 | 1.00 | 42.83 | W | C |
| ATOM | 7707 | CG | GLU | W | 161 | 49.521 | 33.504 | −39.781 | 1.00 | 54.34 | W | C |
| ATOM | 7708 | CD | GLU | W | 161 | 50.362 | 33.422 | −38.512 | 1.00 | 61.19 | W | C |
| ATOM | 7709 | OE1 | GLU | W | 161 | 51.151 | 34.360 | −38.230 | 1.00 | 68.20 | W | O |
| ATOM | 7710 | OE2 | GLU | W | 161 | 50.231 | 32.410 | −37.791 | 1.00 | 61.75 | W | O |
| ATOM | 7711 | C | GLU | W | 161 | 48.457 | 34.054 | −42.672 | 1.00 | 39.91 | W | C |
| ATOM | 7712 | O | GLU | W | 161 | 48.712 | 35.154 | −43.171 | 1.00 | 39.53 | W | O |
| ATOM | 7713 | N | THR | W | 162 | 47.214 | 33.618 | −42.464 | 1.00 | 32.87 | W | N |
| ATOM | 7714 | CA | THR | W | 162 | 46.045 | 34.414 | −42.808 | 1.00 | 27.03 | W | C |
| ATOM | 7715 | CB | THR | W | 162 | 45.238 | 33.708 | −43.902 | 1.00 | 29.81 | W | C |
| ATOM | 7716 | OG1 | THR | W | 162 | 46.098 | 33.415 | −45.008 | 1.00 | 28.47 | W | O |
| ATOM | 7717 | CG2 | THR | W | 162 | 44.096 | 34.584 | −44.386 | 1.00 | 32.25 | W | C |
| ATOM | 7718 | C | THR | W | 162 | 45.136 | 34.696 | −41.610 | 1.00 | 27.24 | W | C |
| ATOM | 7719 | O | THR | W | 162 | 44.911 | 33.835 | −40.772 | 1.00 | 23.24 | W | O |
| ATOM | 7720 | N | THR | W | 163 | 44.637 | 35.922 | −41.523 | 1.00 | 22.08 | W | N |
| ATOM | 7721 | CA | THR | W | 163 | 43.758 | 36.310 | −40.437 | 1.00 | 28.68 | W | C |
| ATOM | 7722 | CB | THR | W | 163 | 43.706 | 37.843 | −40.276 | 1.00 | 29.04 | W | C |
| ATOM | 7723 | OG1 | THR | W | 163 | 43.235 | 38.445 | −41.486 | 1.00 | 24.46 | W | O |
| ATOM | 7724 | CG2 | THR | W | 163 | 45.086 | 38.392 | −39.956 | 1.00 | 28.21 | W | C |
| ATOM | 7725 | C | THR | W | 163 | 42.335 | 35.806 | −40.667 | 1.00 | 35.96 | W | C |
| ATOM | 7726 | O | THR | W | 163 | 41.911 | 35.570 | −41.801 | 1.00 | 32.12 | W | O |
| ATOM | 7727 | N | GLN | W | 164 | 41.609 | 35.628 | −39.574 | 1.00 | 36.98 | W | N |
| ATOM | 7728 | CA | GLN | W | 164 | 40.232 | 35.175 | −39.635 | 1.00 | 37.96 | W | C |
| ATOM | 7729 | CB | GLN | W | 164 | 39.797 | 34.716 | −38.234 | 1.00 | 43.59 | W | C |
| ATOM | 7730 | CG | GLN | W | 164 | 38.575 | 33.802 | −38.164 | 1.00 | 55.54 | W | C |
| ATOM | 7731 | CD | GLN | W | 164 | 38.818 | 32.426 | −38.775 | 1.00 | 60.82 | W | C |
| ATOM | 7732 | OE1 | GLN | W | 164 | 39.874 | 32.166 | −39.368 | 1.00 | 60.83 | W | O |
| ATOM | 7733 | NE2 | GLN | W | 164 | 37.832 | 31.538 | −38.637 | 1.00 | 61.45 | W | N |
| ATOM | 7734 | C | GLN | W | 164 | 39.438 | 36.413 | −40.069 | 1.00 | 37.61 | W | C |
| ATOM | 7735 | O | GLN | W | 164 | 39.721 | 37.521 | −39.622 | 1.00 | 39.22 | W | O |
| ATOM | 7736 | N | PRO | W | 165 | 38.496 | 36.256 | −41.014 | 1.00 | 37.13 | W | N |
| ATOM | 7737 | CD | PRO | W | 165 | 38.214 | 35.039 | −41.802 | 1.00 | 31.16 | W | C |
| ATOM | 7738 | CA | PRO | W | 165 | 37.692 | 37.398 | −41.478 | 1.00 | 33.77 | W | C |
| ATOM | 7739 | CB | PRO | W | 165 | 36.708 | 36.743 | −42.454 | 1.00 | 32.87 | W | C |
| ATOM | 7740 | CG | PRO | W | 165 | 37.508 | 35.593 | −43.021 | 1.00 | 33.51 | W | C |
| ATOM | 7741 | C | PRO | W | 165 | 36.952 | 38.149 | −40.349 | 1.00 | 36.83 | W | C |
| ATOM | 7742 | O | PRO | W | 165 | 36.387 | 37.540 | −39.430 | 1.00 | 33.75 | W | O |
| ATOM | 7743 | N | SER | W | 166 | 36.985 | 39.475 | −40.411 | 1.00 | 33.28 | W | N |
| ATOM | 7744 | CA | SER | W | 166 | 36.312 | 40.311 | −39.422 | 1.00 | 37.50 | W | C |
| ATOM | 7745 | CB | SER | W | 166 | 37.326 | 41.067 | −38.546 | 1.00 | 40.15 | W | C |
| ATOM | 7746 | OG | SER | W | 166 | 38.134 | 41.953 | −39.304 | 1.00 | 40.06 | W | O |
| ATOM | 7747 | C | SER | W | 166 | 35.498 | 41.284 | −40.242 | 1.00 | 40.51 | W | C |
| ATOM | 7748 | O | SER | W | 166 | 35.883 | 41.591 | −41.374 | 1.00 | 33.93 | W | O |
| ATOM | 7749 | N | LYS | W | 167 | 34.360 | 41.747 | −39.717 | 1.00 | 45.18 | W | N |
| ATOM | 7750 | CA | LYS | W | 167 | 33.563 | 42.670 | −40.513 | 1.00 | 42.74 | W | C |
| ATOM | 7751 | CB | LYS | W | 167 | 32.052 | 42.357 | −40.515 | 1.00 | 51.30 | W | C |
| ATOM | 7752 | CG | LYS | W | 167 | 31.332 | 42.070 | −39.204 | 1.00 | 53.94 | W | C |
| ATOM | 7753 | CD | LYS | W | 167 | 29.841 | 41.804 | −39.524 | 1.00 | 46.98 | W | C |
| ATOM | 7754 | CE | LYS | W | 167 | 29.021 | 41.410 | −38.297 | 1.00 | 53.61 | W | C |
| ATOM | 7755 | NZ | LYS | W | 167 | 28.942 | 42.474 | −37.245 | 1.00 | 51.76 | W | N |
| ATOM | 7756 | C | LYS | W | 167 | 33.861 | 44.142 | −40.388 | 1.00 | 39.15 | W | C |
| ATOM | 7757 | O | LYS | W | 167 | 34.209 | 44.631 | −39.320 | 1.00 | 47.67 | W | O |
| ATOM | 7758 | N | GLN | W | 168 | 33.852 | 44.812 | −41.535 | 1.00 | 36.79 | W | N |
| ATOM | 7759 | CA | GLN | W | 168 | 34.127 | 46.236 | −41.599 | 1.00 | 39.63 | W | C |
| ATOM | 7760 | CB | GLN | W | 168 | 34.738 | 46.632 | −42.954 | 1.00 | 36.41 | W | C |
| ATOM | 7761 | CG | GLN | W | 168 | 34.269 | 45.820 | −44.137 | 1.00 | 36.39 | W | C |
| ATOM | 7762 | CD | GLN | W | 168 | 35.146 | 46.004 | −45.371 | 1.00 | 37.84 | W | C |
| ATOM | 7763 | OE1 | GLN | W | 168 | 36.117 | 45.272 | −45.570 | 1.00 | 35.75 | W | O |
| ATOM | 7764 | NE2 | GLN | W | 168 | 34.788 | 46.964 | −46.219 | 1.00 | 39.10 | W | N |
| ATOM | 7765 | C | GLN | W | 168 | 32.884 | 47.039 | −41.276 | 1.00 | 43.20 | W | C |
| ATOM | 7766 | O | GLN | W | 168 | 31.860 | 46.467 | −40.899 | 1.00 | 47.56 | W | O |
| ATOM | 7767 | N | SER | W | 168A | 32.970 | 48.360 | −41.401 | 1.00 | 47.46 | W | N |
| ATOM | 7768 | CA | SER | W | 168A | 31.832 | 49.205 | −41.073 | 1.00 | 52.14 | W | C |
| ATOM | 7769 | CB | SER | W | 168A | 32.241 | 50.685 | −40.973 | 1.00 | 53.77 | W | C |
| ATOM | 7770 | OG | SER | W | 168A | 32.821 | 51.167 | −42.170 | 1.00 | 56.23 | W | O |
| ATOM | 7771 | C | SER | W | 168A | 30.616 | 49.008 | −41.980 | 1.00 | 56.02 | W | C |
| ATOM | 7772 | O | SER | W | 168A | 29.484 | 49.096 | −41.506 | 1.00 | 65.24 | W | O |
| ATOM | 7773 | N | ASN | W | 168B | 30.834 | 48.693 | −43.259 | 1.00 | 56.01 | W | N |
| ATOM | 7774 | CA | ASN | W | 168B | 29.719 | 48.474 | −44.184 | 1.00 | 48.40 | W | C |
| ATOM | 7775 | CB | ASN | W | 168B | 30.098 | 48.892 | −45.613 | 1.00 | 50.41 | W | C |
| ATOM | 7776 | CG | ASN | W | 168B | 31.130 | 47.967 | −46.250 | 1.00 | 52.05 | W | C |
| ATOM | 7777 | OD1 | ASN | W | 168B | 31.443 | 46.894 | −45.722 | 1.00 | 46.59 | W | O |
| ATOM | 7778 | ND2 | ASN | W | 168B | 31.653 | 48.378 | −47.399 | 1.00 | 47.84 | W | N |
| ATOM | 7779 | C | ASN | W | 168B | 29.157 | 47.037 | −44.146 | 1.00 | 49.21 | W | C |
| ATOM | 7780 | O | ASN | W | 168B | 28.411 | 46.626 | −45.034 | 1.00 | 51.23 | W | O |
| ATOM | 7781 | N | ASN | W | 169 | 29.504 | 46.297 | −43.096 | 1.00 | 49.97 | W | N |
| ATOM | 7782 | CA | ASN | W | 169 | 29.059 | 44.913 | −42.891 | 1.00 | 48.33 | W | C |
| ATOM | 7783 | CB | ASN | W | 169 | 27.521 | 44.800 | −42.822 | 1.00 | 56.12 | W | C |
| ATOM | 7784 | CG | ASN | W | 169 | 26.966 | 45.042 | −41.406 | 1.00 | 56.71 | W | C |
| ATOM | 7785 | OD1 | ASN | W | 169 | 27.127 | 44.208 | −40.504 | 1.00 | 54.82 | W | O |
| ATOM | 7786 | ND2 | ASN | W | 169 | 26.309 | 46.183 | −41.216 | 1.00 | 55.29 | W | N |
| ATOM | 7787 | C | ASN | W | 169 | 29.665 | 43.844 | −43.818 | 1.00 | 46.44 | W | C |
| ATOM | 7788 | O | ASN | W | 169 | 29.352 | 42.657 | −43.686 | 1.00 | 42.22 | W | O |
| ATOM | 7789 | N | LYS | W | 170 | 30.497 | 44.257 | −44.776 | 1.00 | 40.04 | W | N |
| ATOM | 7790 | CA | LYS | W | 170 | 31.190 | 43.281 | −45.618 | 1.00 | 36.90 | W | C |
| ATOM | 7791 | CB | LYS | W | 170 | 31.701 | 43.908 | −46.910 | 1.00 | 30.17 | W | C |
| ATOM | 7792 | CG | LYS | W | 170 | 30.625 | 44.456 | −47.818 | 1.00 | 29.71 | W | C |
| ATOM | 7793 | CD | LYS | W | 170 | 31.240 | 44.953 | −49.119 | 1.00 | 24.95 | W | C |
| ATOM | 7794 | CE | LYS | W | 170 | 30.219 | 45.722 | −49.939 | 1.00 | 33.32 | W | C |
| ATOM | 7795 | NZ | LYS | W | 170 | 30.702 | 46.073 | −51.308 | 1.00 | 30.50 | W | N |
| ATOM | 7796 | C | LYS | W | 170 | 32.383 | 42.808 | −44.760 | 1.00 | 36.29 | W | C |
| ATOM | 7797 | O | LYS | W | 170 | 32.503 | 43.192 | −43.599 | 1.00 | 34.73 | W | O |
| ATOM | 7798 | N | TYR | W | 171 | 33.267 | 41.989 | −45.308 | 1.00 | 29.30 | W | N |
| ATOM | 7799 | CA | TYR | W | 171 | 34.387 | 41.514 | −44.516 | 1.00 | 27.02 | W | C |
| ATOM | 7800 | CB | TYR | W | 171 | 34.287 | 40.008 | −44.328 | 1.00 | 28.60 | W | C |
| ATOM | 7801 | CG | TYR | W | 171 | 33.106 | 39.570 | −43.493 | 1.00 | 34.28 | W | C |
| ATOM | 7802 | CD1 | TYR | W | 171 | 31.793 | 39.847 | −43.886 | 1.00 | 33.33 | W | C |
| ATOM | 7803 | CE1 | TYR | W | 171 | 30.715 | 39.464 | −43.096 | 1.00 | 31.08 | W | C |
| ATOM | 7804 | CD2 | TYR | W | 171 | 33.303 | 38.892 | −42.292 | 1.00 | 34.11 | W | C |
| ATOM | 7805 | CE2 | TYR | W | 171 | 32.234 | 38.502 | −41.501 | 1.00 | 31.25 | W | C |
| ATOM | 7806 | CZ | TYR | W | 171 | 30.947 | 38.790 | −41.902 | 1.00 | 32.92 | W | C |
| ATOM | 7807 | OH | TYR | W | 171 | 29.897 | 38.395 | −41.097 | 1.00 | 35.88 | W | O |
| ATOM | 7808 | C | TYR | W | 171 | 35.765 | 41.890 | −45.061 | 1.00 | 28.84 | W | C |
| ATOM | 7809 | O | TYR | W | 171 | 35.921 | 42.208 | −46.239 | 1.00 | 30.25 | W | O |
| ATOM | 7810 | N | MET | W | 172 | 36.753 | 41.854 | −44.175 | 1.00 | 26.80 | W | N |
| ATOM | 7811 | CA | MET | W | 172 | 38.137 | 42.174 | −44.487 | 1.00 | 25.52 | W | C |
| ATOM | 7812 | CB | MET | W | 172 | 38.519 | 43.535 | −43.902 | 1.00 | 29.40 | W | C |
| ATOM | 7813 | CG | MET | W | 172 | 38.548 | 43.559 | −42.384 | 1.00 | 31.91 | W | C |
| ATOM | 7814 | SD | MET | W | 172 | 39.111 | 45.127 | −41.739 | 1.00 | 42.76 | W | S |
| ATOM | 7815 | CE | MET | W | 172 | 37.614 | 45.782 | −41.020 | 1.00 | 41.78 | W | C |
| ATOM | 7816 | C | MET | W | 172 | 39.008 | 41.090 | −43.861 | 1.00 | 24.80 | W | C |
| ATOM | 7817 | O | MET | W | 172 | 38.615 | 40.445 | −42.897 | 1.00 | 29.23 | W | O |
| ATOM | 7818 | N | ALA | W | 173 | 40.188 | 40.882 | −44.426 | 1.00 | 20.09 | W | N |
| ATOM | 7819 | CA | ALA | W | 173 | 41.099 | 39.867 | −43.926 | 1.00 | 17.13 | W | C |
| ATOM | 7820 | CB | ALA | W | 173 | 40.627 | 38.492 | −44.361 | 1.00 | 14.84 | W | C |
| ATOM | 7821 | C | ALA | W | 173 | 42.497 | 40.158 | −44.460 | 1.00 | 15.28 | W | C |
| ATOM | 7822 | O | ALA | W | 173 | 42.661 | 41.016 | −45.309 | 1.00 | 16.96 | W | O |
| ATOM | 7823 | N | SER | W | 174 | 43.504 | 39.434 | −43.991 | 1.00 | 14.19 | W | N |
| ATOM | 7824 | CA | SER | W | 174 | 44.855 | 39.699 | −44.450 | 1.00 | 12.49 | W | C |
| ATOM | 7825 | CB | SER | W | 174 | 45.487 | 40.762 | −43.558 | 1.00 | 22.34 | W | C |
| ATOM | 7826 | OG | SER | W | 174 | 45.539 | 40.324 | −42.198 | 1.00 | 23.85 | W | O |
| ATOM | 7827 | C | SER | W | 174 | 45.697 | 38.457 | −44.396 | 1.00 | 18.41 | W | C |
| ATOM | 7828 | O | SER | W | 174 | 45.463 | 37.578 | −43.564 | 1.00 | 17.77 | W | O |
| ATOM | 7829 | N | SER | W | 175 | 46.655 | 38.368 | −45.311 | 1.00 | 14.92 | W | N |
| ATOM | 7830 | CA | SER | W | 175 | 47.563 | 37.231 | −45.337 | 1.00 | 20.25 | W | C |
| ATOM | 7831 | CB | SER | W | 175 | 47.218 | 36.257 | −46.450 | 1.00 | 19.38 | W | C |
| ATOM | 7832 | OG | SER | W | 175 | 47.985 | 35.063 | −46.307 | 1.00 | 30.59 | W | O |
| ATOM | 7833 | C | SER | W | 175 | 49.017 | 37.685 | −45.477 | 1.00 | 21.87 | W | C |
| ATOM | 7834 | O | SER | W | 175 | 49.322 | 38.693 | −46.124 | 1.00 | 20.89 | W | O |
| ATOM | 7835 | N | TYR | W | 176 | 49.911 | 36.898 | −44.901 | 1.00 | 21.03 | W | N |
| ATOM | 7836 | CA | TYR | W | 176 | 51.321 | 37.218 | −44.917 | 1.00 | 21.07 | W | C |
| ATOM | 7837 | CB | TYR | W | 176 | 51.778 | 37.503 | −43.479 | 1.00 | 18.41 | W | C |
| ATOM | 7838 | CG | TYR | W | 176 | 51.178 | 38.736 | −42.853 | 1.00 | 17.26 | W | C |
| ATOM | 7839 | CD1 | TYR | W | 176 | 51.500 | 39.997 | −43.323 | 1.00 | 15.32 | W | C |
| ATOM | 7840 | CE1 | TYR | W | 176 | 50.965 | 41.133 | −42.752 | 1.00 | 16.40 | W | C |
| ATOM | 7841 | CD2 | TYR | W | 176 | 50.302 | 38.641 | −41.782 | 1.00 | 17.95 | W | C |
| ATOM | 7842 | CE2 | TYR | W | 176 | 49.771 | 39.776 | −41.199 | 1.00 | 19.12 | W | C |
| ATOM | 7843 | CZ | TYR | W | 176 | 50.105 | 41.017 | −41.696 | 1.00 | 17.37 | W | C |
| ATOM | 7844 | OH | TYR | W | 176 | 49.544 | 42.151 | −41.162 | 1.00 | 25.32 | W | O |
| ATOM | 7845 | C | TYR | W | 176 | 52.196 | 36.107 | −45.461 | 1.00 | 20.15 | W | C |
| ATOM | 7846 | O | TYR | W | 176 | 52.043 | 34.948 | −45.087 | 1.00 | 23.36 | W | O |
| ATOM | 7847 | N | LEU | W | 177 | 53.149 | 36.457 | −46.310 | 1.00 | 21.85 | W | N |
| ATOM | 7848 | CA | LEU | W | 177 | 54.074 | 35.450 | −46.783 | 1.00 | 21.08 | W | C |
| ATOM | 7849 | CB | LEU | W | 177 | 54.043 | 35.295 | −48.299 | 1.00 | 23.53 | W | C |
| ATOM | 7850 | CG | LEU | W | 177 | 54.981 | 34.183 | −48.809 | 1.00 | 31.46 | W | C |
| ATOM | 7851 | CD1 | LEU | W | 177 | 54.957 | 32.998 | −47.858 | 1.00 | 27.07 | W | C |
| ATOM | 7852 | CD2 | LEU | W | 177 | 54.623 | 33.745 | −50.244 | 1.00 | 26.77 | W | C |
| ATOM | 7853 | C | LEU | W | 177 | 55.439 | 35.888 | −46.266 | 1.00 | 24.98 | W | C |
| ATOM | 7854 | O | LEU | W | 177 | 55.947 | 36.956 | −46.602 | 1.00 | 23.00 | W | O |
| ATOM | 7855 | N | THR | W | 178 | 55.970 | 35.077 | −45.366 | 1.00 | 30.05 | W | N |
| ATOM | 7856 | CA | THR | W | 178 | 57.249 | 35.316 | −44.721 | 1.00 | 31.39 | W | C |
| ATOM | 7857 | CB | THR | W | 178 | 57.177 | 34.866 | −43.240 | 1.00 | 33.72 | W | C |
| ATOM | 7858 | OG1 | THR | W | 178 | 56.110 | 35.568 | −42.580 | 1.00 | 31.27 | W | O |
| ATOM | 7859 | CG2 | THR | W | 178 | 58.501 | 35.139 | −42.526 | 1.00 | 31.23 | W | C |
| ATOM | 7860 | C | THR | W | 178 | 58.407 | 34.587 | −45.405 | 1.00 | 34.33 | W | C |
| ATOM | 7861 | O | THR | W | 178 | 58.456 | 33.356 | −45.432 | 1.00 | 32.44 | W | O |
| ATOM | 7862 | N | LEU | W | 179 | 59.358 | 35.367 | −45.908 | 1.00 | 36.51 | W | N |
| ATOM | 7863 | CA | LEU | W | 179 | 60.540 | 34.846 | −46.584 | 1.00 | 38.77 | W | C |
| ATOM | 7864 | CB | LEU | W | 179 | 60.472 | 35.191 | −48.076 | 1.00 | 39.37 | W | C |
| ATOM | 7865 | CG | LEU | W | 179 | 59.294 | 34.781 | −48.957 | 1.00 | 41.91 | W | C |
| ATOM | 7866 | CD1 | LEU | W | 179 | 59.294 | 35.664 | −50.185 | 1.00 | 39.09 | W | C |
| ATOM | 7867 | CD2 | LEU | W | 179 | 59.374 | 33.306 | −49.344 | 1.00 | 41.53 | W | C |
| ATOM | 7868 | C | LEU | W | 179 | 61.783 | 35.537 | −46.024 | 1.00 | 38.92 | W | C |
| ATOM | 7869 | O | LEU | W | 179 | 61.691 | 36.570 | −45.354 | 1.00 | 38.69 | W | O |
| ATOM | 7870 | N | THR | W | 180 | 62.948 | 34.961 | −46.295 | 1.00 | 35.72 | W | N |
| ATOM | 7871 | CA | THR | W | 180 | 64.203 | 35.586 | −45.889 | 1.00 | 30.77 | W | C |
| ATOM | 7872 | CB | THR | W | 180 | 65.356 | 34.557 | −45.782 | 1.00 | 31.56 | W | C |
| ATOM | 7873 | OG1 | THR | W | 180 | 65.552 | 33.913 | −47.051 | 1.00 | 30.98 | W | O |
| ATOM | 7874 | CG2 | THR | W | 180 | 65.060 | 33.521 | −44.714 | 1.00 | 16.52 | W | C |
| ATOM | 7875 | C | THR | W | 180 | 64.500 | 36.500 | −47.070 | 1.00 | 31.54 | W | C |
| ATOM | 7876 | O | THR | W | 180 | 63.922 | 36.313 | −48.148 | 1.00 | 30.66 | W | O |
| ATOM | 7877 | N | ALA | W | 181 | 65.390 | 37.473 | −46.893 | 1.00 | 30.99 | W | N |
| ATOM | 7878 | CA | ALA | W | 181 | 65.730 | 38.364 | −48.008 | 1.00 | 31.15 | W | C |
| ATOM | 7879 | CB | ALA | W | 181 | 66.703 | 39.472 | −47.566 | 1.00 | 20.89 | W | C |
| ATOM | 7880 | C | ALA | W | 181 | 66.326 | 37.553 | −49.171 | 1.00 | 28.76 | W | C |
| ATOM | 7881 | O | ALA | W | 181 | 66.164 | 37.925 | −50.331 | 1.00 | 31.96 | W | O |
| ATOM | 7882 | N | GLU | W | 182 | 66.975 | 36.434 | −48.848 | 1.00 | 35.02 | W | N |
| ATOM | 7883 | CA | GLU | W | 182 | 67.587 | 35.564 | −49.855 | 1.00 | 41.60 | W | C |
| ATOM | 7884 | CB | GLU | W | 182 | 68.406 | 34.438 | −49.202 | 1.00 | 47.87 | W | C |
| ATOM | 7885 | CG | GLU | W | 182 | 69.817 | 34.843 | −48.800 | 1.00 | 53.41 | W | C |
| ATOM | 7886 | CD | GLU | W | 182 | 70.605 | 35.457 | −49.952 | 1.00 | 60.20 | W | C |
| ATOM | 7887 | OE1 | GLU | W | 182 | 71.246 | 34.691 | −50.710 | 1.00 | 61.80 | W | O |
| ATOM | 7888 | OE2 | GLU | W | 182 | 70.585 | 36.706 | −50.095 | 1.00 | 59.91 | W | O |
| ATOM | 7889 | C | GLU | W | 182 | 66.543 | 34.968 | −50.772 | 1.00 | 34.75 | W | C |
| ATOM | 7890 | O | GLU | W | 182 | 66.632 | 35.094 | −51.995 | 1.00 | 33.31 | W | O |
| ATOM | 7891 | N | ALA | W | 183 | 65.547 | 34.325 | −50.173 | 1.00 | 36.11 | W | N |
| ATOM | 7892 | CA | ALA | W | 183 | 64.469 | 33.718 | −50.948 | 1.00 | 29.36 | W | C |
| ATOM | 7893 | CB | ALA | W | 183 | 63.479 | 33.027 | −50.044 | 1.00 | 27.69 | W | C |
| ATOM | 7894 | C | ALA | W | 183 | 63.770 | 34.766 | −51.797 | 1.00 | 29.76 | W | C |
| ATOM | 7895 | O | ALA | W | 183 | 63.423 | 34.483 | −52.939 | 1.00 | 31.86 | W | O |
| ATOM | 7896 | N | TRP | W | 184 | 63.576 | 35.975 | −51.263 | 1.00 | 22.80 | W | N |
| ATOM | 7897 | CA | TRP | W | 184 | 62.923 | 37.026 | −52.038 | 1.00 | 27.82 | W | C |
| ATOM | 7898 | CB | TRP | W | 184 | 62.620 | 38.260 | −51.175 | 1.00 | 22.04 | W | C |
| ATOM | 7899 | CG | TRP | W | 184 | 62.341 | 39.526 | −51.958 | 1.00 | 20.26 | W | C |
| ATOM | 7900 | CD2 | TRP | W | 184 | 61.129 | 39.881 | −52.658 | 1.00 | 24.50 | W | C |
| ATOM | 7901 | CE2 | TRP | W | 184 | 61.344 | 41.150 | −53.244 | 1.00 | 20.94 | W | C |
| ATOM | 7902 | CE3 | TRP | W | 184 | 59.887 | 39.249 | −52.850 | 1.00 | 25.89 | W | C |
| ATOM | 7903 | CD1 | TRP | W | 184 | 63.203 | 40.562 | −52.150 | 1.00 | 26.58 | W | C |
| ATOM | 7904 | NE1 | TRP | W | 184 | 62.616 | 41.537 | −52.920 | 1.00 | 28.93 | W | N |
| ATOM | 7905 | CZ2 | TRP | W | 184 | 60.365 | 41.808 | −54.002 | 1.00 | 20.25 | W | C |
| ATOM | 7906 | CZ3 | TRP | W | 184 | 58.900 | 39.912 | −53.616 | 1.00 | 18.30 | W | C |
| ATOM | 7907 | CH2 | TRP | W | 184 | 59.153 | 41.172 | −54.178 | 1.00 | 22.02 | W | C |
| ATOM | 7908 | C | TRP | W | 184 | 63.739 | 37.411 | −53.273 | 1.00 | 30.29 | W | C |
| ATOM | 7909 | O | TRP | W | 184 | 63.180 | 37.687 | −54.341 | 1.00 | 32.47 | W | O |
| ATOM | 7910 | N | GLU | W | 185 | 65.059 | 37.376 | −53.136 | 1.00 | 34.77 | W | N |
| ATOM | 7911 | CA | GLU | W | 185 | 65.963 | 37.736 | −54.227 | 1.00 | 37.01 | W | C |
| ATOM | 7912 | CB | GLU | W | 185 | 67.358 | 38.076 | −53.671 | 1.00 | 42.77 | W | C |
| ATOM | 7913 | CG | GLU | W | 185 | 67.436 | 39.342 | −52.806 | 1.00 | 51.65 | W | C |
| ATOM | 7914 | CD | GLU | W | 185 | 68.806 | 39.534 | −52.144 | 1.00 | 57.58 | W | C |
| ATOM | 7915 | OE1 | GLU | W | 185 | 69.761 | 39.961 | −52.835 | 1.00 | 60.47 | W | O |
| ATOM | 7916 | OE2 | GLU | W | 185 | 68.930 | 39.263 | −50.928 | 1.00 | 60.88 | W | O |
| ATOM | 7917 | C | GLU | W | 185 | 66.093 | 36.626 | −55.265 | 1.00 | 37.47 | W | C |
| ATOM | 7918 | O | GLU | W | 185 | 66.261 | 36.898 | −56.446 | 1.00 | 32.45 | W | O |
| ATOM | 7919 | N | SER | W | 186 | 66.010 | 35.376 | −54.821 | 1.00 | 39.87 | W | N |
| ATOM | 7920 | CA | SER | W | 186 | 66.152 | 34.237 | −55.725 | 1.00 | 41.98 | W | C |
| ATOM | 7921 | CB | SER | W | 186 | 66.602 | 32.999 | −54.942 | 1.00 | 42.89 | W | C |
| ATOM | 7922 | OG | SER | W | 186 | 67.914 | 33.195 | −54.407 | 1.00 | 39.09 | W | O |
| ATOM | 7923 | C | SER | W | 186 | 64.951 | 33.931 | −56.636 | 1.00 | 41.33 | W | C |
| ATOM | 7924 | O | SER | W | 186 | 65.115 | 33.345 | −57.703 | 1.00 | 40.42 | W | O |
| ATOM | 7925 | N | HIS | W | 187 | 63.760 | 34.379 | −56.248 | 1.00 | 41.00 | W | N |
| ATOM | 7926 | CA | HIS | W | 187 | 62.550 | 34.147 | −57.047 | 1.00 | 38.13 | W | C |
| ATOM | 7927 | CB | HIS | W | 187 | 61.479 | 33.505 | −56.182 | 1.00 | 31.52 | W | C |
| ATOM | 7928 | CG | HIS | W | 187 | 61.947 | 32.261 | −55.494 | 1.00 | 37.62 | W | C |
| ATOM | 7929 | CD2 | HIS | W | 187 | 61.849 | 30.957 | −55.844 | 1.00 | 39.07 | W | C |
| ATOM | 7930 | ND1 | HIS | W | 187 | 62.650 | 32.286 | −54.309 | 1.00 | 42.36 | W | N |
| ATOM | 7931 | CE1 | HIS | W | 187 | 62.968 | 31.053 | −53.958 | 1.00 | 39.65 | W | C |
| ATOM | 7932 | NE2 | HIS | W | 187 | 62.494 | 30.228 | −54.873 | 1.00 | 41.46 | W | N |
| ATOM | 7933 | C | HIS | W | 187 | 62.080 | 35.466 | −57.644 | 1.00 | 36.57 | W | C |
| ATOM | 7934 | O | HIS | W | 187 | 62.488 | 36.524 | −57.163 | 1.00 | 41.19 | W | O |
| ATOM | 7935 | N | SER | W | 188 | 61.264 | 35.423 | −58.699 | 1.00 | 33.25 | W | N |
| ATOM | 7936 | CA | SER | W | 188 | 60.818 | 36.665 | −59.333 | 1.00 | 32.18 | W | C |
| ATOM | 7937 | CB | SER | W | 188 | 61.403 | 36.807 | −60.741 | 1.00 | 39.39 | W | C |
| ATOM | 7938 | OG | SER | W | 188 | 60.704 | 36.005 | −61.667 | 1.00 | 45.63 | W | O |
| ATOM | 7939 | C | SER | W | 188 | 59.323 | 36.961 | −59.384 | 1.00 | 32.05 | W | C |
| ATOM | 7940 | O | SER | W | 188 | 58.927 | 38.109 | −59.589 | 1.00 | 32.79 | W | O |
| ATOM | 7941 | N | SER | W | 189 | 58.486 | 35.955 | −59.196 | 1.00 | 30.91 | W | N |
| ATOM | 7942 | CA | SER | W | 189 | 57.059 | 36.203 | −59.235 | 1.00 | 31.02 | W | C |
| ATOM | 7943 | CB | SER | W | 189 | 56.448 | 35.617 | −60.508 | 1.00 | 37.25 | W | C |
| ATOM | 7944 | OG | SER | W | 189 | 55.082 | 35.980 | −60.626 | 1.00 | 40.73 | W | O |
| ATOM | 7945 | C | SER | W | 189 | 56.330 | 35.675 | −58.009 | 1.00 | 33.41 | W | C |
| ATOM | 7946 | O | SER | W | 189 | 56.512 | 34.528 | −57.600 | 1.00 | 29.33 | W | O |
| ATOM | 7947 | N | TYR | W | 190 | 55.503 | 36.533 | −57.423 | 1.00 | 33.74 | W | N |
| ATOM | 7948 | CA | TYR | W | 190 | 54.723 | 36.159 | −56.250 | 1.00 | 34.13 | W | C |
| ATOM | 7949 | CB | TYR | W | 190 | 55.257 | 36.845 | −54.996 | 1.00 | 40.03 | W | C |
| ATOM | 7950 | CG | TYR | W | 190 | 56.584 | 36.301 | −54.540 | 1.00 | 42.38 | W | C |
| ATOM | 7951 | CD1 | TYR | W | 190 | 56.644 | 35.238 | −53.640 | 1.00 | 44.80 | W | C |
| ATOM | 7952 | CE1 | TYR | W | 190 | 57.861 | 34.710 | −53.237 | 1.00 | 44.68 | W | C |
| ATOM | 7953 | CD2 | TYR | W | 190 | 57.780 | 36.828 | −55.026 | 1.00 | 39.62 | W | C |
| ATOM | 7954 | CE2 | TYR | W | 190 | 59.006 | 36.307 | −54.633 | 1.00 | 42.17 | W | C |
| ATOM | 7955 | CZ | TYR | W | 190 | 59.039 | 35.246 | −53.740 | 1.00 | 43.21 | W | C |
| ATOM | 7956 | OH | TYR | W | 190 | 60.248 | 34.700 | −53.361 | 1.00 | 49.15 | W | O |
| ATOM | 7957 | C | TYR | W | 190 | 53.259 | 36.502 | −56.442 | 1.00 | 27.11 | W | C |
| ATOM | 7958 | O | TYR | W | 190 | 52.922 | 37.590 | −56.907 | 1.00 | 27.18 | W | O |
| ATOM | 7959 | N | SER | W | 191 | 52.399 | 35.558 | −56.087 | 1.00 | 19.32 | W | N |
| ATOM | 7960 | CA | SER | W | 191 | 50.965 | 35.749 | −56.217 | 1.00 | 24.36 | W | C |
| ATOM | 7961 | CB | SER | W | 191 | 50.398 | 34.901 | −57.359 | 1.00 | 22.44 | W | C |
| ATOM | 7962 | OG | SER | W | 191 | 50.806 | 35.402 | −58.621 | 1.00 | 28.69 | W | O |
| ATOM | 7963 | C | SER | W | 191 | 50.178 | 35.442 | −54.959 | 1.00 | 23.77 | W | C |
| ATOM | 7964 | O | SER | W | 191 | 50.487 | 34.501 | −54.213 | 1.00 | 21.23 | W | O |
| ATOM | 7965 | N | CYS | W | 192 | 49.177 | 36.280 | −54.722 | 1.00 | 23.53 | W | N |
| ATOM | 7966 | CA | CYS | W | 192 | 48.262 | 36.117 | −53.603 | 1.00 | 23.84 | W | C |
| ATOM | 7967 | C | CYS | W | 192 | 46.934 | 35.748 | −54.261 | 1.00 | 21.32 | W | C |
| ATOM | 7968 | O | CYS | W | 192 | 46.390 | 36.522 | −55.056 | 1.00 | 19.80 | W | O |
| ATOM | 7969 | CB | CYS | W | 192 | 48.086 | 37.421 | −52.793 | 1.00 | 25.63 | W | C |
| ATOM | 7970 | SG | CYS | W | 192 | 46.934 | 37.142 | −51.409 | 1.00 | 35.75 | W | S |
| ATOM | 7971 | N | HIS | W | 193 | 46.459 | 34.546 | −53.946 | 1.00 | 25.93 | W | N |
| ATOM | 7972 | CA | HIS | W | 193 | 45.206 | 33.964 | −54.440 | 1.00 | 21.78 | W | C |
| ATOM | 7973 | CB | HIS | W | 193 | 45.394 | 32.468 | −54.622 | 1.00 | 28.16 | W | C |
| ATOM | 7974 | CG | HIS | W | 193 | 46.017 | 32.057 | −55.912 | 1.00 | 30.85 | W | C |
| ATOM | 7975 | CD2 | HIS | W | 193 | 46.089 | 32.679 | −57.110 | 1.00 | 35.72 | W | C |
| ATOM | 7976 | ND1 | HIS | W | 193 | 46.493 | 30.775 | −56.114 | 1.00 | 29.20 | W | N |
| ATOM | 7977 | CE1 | HIS | W | 193 | 46.808 | 30.623 | −57.385 | 1.00 | 31.74 | W | C |
| ATOM | 7978 | NE2 | HIS | W | 193 | 46.575 | 31.761 | −58.013 | 1.00 | 35.24 | W | N |
| ATOM | 7979 | C | HIS | W | 193 | 44.121 | 34.059 | −53.371 | 1.00 | 24.25 | W | C |
| ATOM | 7980 | O | HIS | W | 193 | 44.290 | 33.510 | −52.277 | 1.00 | 25.10 | W | O |
| ATOM | 7981 | N | VAL | W | 194 | 42.984 | 34.667 | −53.697 | 1.00 | 23.10 | W | N |
| ATOM | 7982 | CA | VAL | W | 194 | 41.885 | 34.746 | −52.745 | 1.00 | 18.25 | W | C |
| ATOM | 7983 | CB | VAL | W | 194 | 41.608 | 36.199 | −52.257 | 1.00 | 21.45 | W | C |
| ATOM | 7984 | CG1 | VAL | W | 194 | 40.527 | 36.191 | −51.181 | 1.00 | 11.12 | W | C |
| ATOM | 7985 | CG2 | VAL | W | 194 | 42.892 | 36.837 | −51.690 | 1.00 | 16.77 | W | C |
| ATOM | 7986 | C | VAL | W | 194 | 40.647 | 34.180 | −53.446 | 1.00 | 26.75 | W | C |
| ATOM | 7987 | O | VAL | W | 194 | 40.188 | 34.721 | −54.454 | 1.00 | 18.16 | W | O |
| ATOM | 7988 | N | THR | W | 195 | 40.139 | 33.071 | −52.918 | 1.00 | 23.81 | W | N |
| ATOM | 7989 | CA | THR | W | 195 | 38.974 | 32.401 | −53.471 | 1.00 | 21.71 | W | C |
| ATOM | 7990 | CB | THR | W | 195 | 39.200 | 30.892 | −53.592 | 1.00 | 22.79 | W | C |
| ATOM | 7991 | OG1 | THR | W | 195 | 40.234 | 30.647 | −54.562 | 1.00 | 20.07 | W | O |
| ATOM | 7992 | CG2 | THR | W | 195 | 37.904 | 30.198 | −54.016 | 1.00 | 16.27 | W | C |
| ATOM | 7993 | C | THR | W | 195 | 37.749 | 32.666 | −52.621 | 1.00 | 25.41 | W | C |
| ATOM | 7994 | O | THR | W | 195 | 37.753 | 32.411 | −51.424 | 1.00 | 17.47 | W | O |
| ATOM | 7995 | N | HIS | W | 196 | 36.694 | 33.127 | −53.282 | 1.00 | 19.40 | W | N |
| ATOM | 7996 | CA | HIS | W | 196 | 35.443 | 33.489 | −52.651 | 1.00 | 19.57 | W | C |
| ATOM | 7997 | CB | HIS | W | 196 | 35.373 | 35.013 | −52.624 | 1.00 | 21.45 | W | C |
| ATOM | 7998 | CG | HIS | W | 196 | 34.091 | 35.561 | −52.100 | 1.00 | 17.07 | W | C |
| ATOM | 7999 | CD2 | HIS | W | 196 | 33.592 | 35.616 | −50.842 | 1.00 | 20.87 | W | C |
| ATOM | 8000 | ND1 | HIS | W | 196 | 33.190 | 36.224 | −52.902 | 1.00 | 20.37 | W | N |
| ATOM | 8001 | CE1 | HIS | W | 196 | 32.194 | 36.674 | −52.159 | 1.00 | 19.92 | W | C |
| ATOM | 8002 | NE2 | HIS | W | 196 | 32.414 | 36.320 | −50.906 | 1.00 | 20.07 | W | N |
| ATOM | 8003 | C | HIS | W | 196 | 34.315 | 32.946 | −53.512 | 1.00 | 24.68 | W | C |
| ATOM | 8004 | O | HIS | W | 196 | 34.259 | 33.222 | −54.715 | 1.00 | 22.73 | W | O |
| ATOM | 8005 | N | GLU | W | 197 | 33.416 | 32.183 | −52.901 | 1.00 | 25.29 | W | N |
| ATOM | 8006 | CA | GLU | W | 197 | 32.294 | 31.602 | −53.635 | 1.00 | 29.60 | W | C |
| ATOM | 8007 | CB | GLU | W | 197 | 31.332 | 32.702 | −54.123 | 1.00 | 32.45 | W | C |
| ATOM | 8008 | CG | GLU | W | 197 | 30.619 | 33.521 | −53.038 | 1.00 | 40.55 | W | C |
| ATOM | 8009 | CD | GLU | W | 197 | 29.361 | 32.850 | −52.479 | 1.00 | 45.95 | W | C |
| ATOM | 8010 | OE1 | GLU | W | 197 | 28.771 | 31.993 | −53.175 | 1.00 | 56.48 | W | O |
| ATOM | 8011 | OE2 | GLU | W | 197 | 28.939 | 33.194 | −51.348 | 1.00 | 51.30 | W | O |
| ATOM | 8012 | C | GLU | W | 197 | 32.813 | 30.825 | −54.855 | 1.00 | 31.54 | W | C |
| ATOM | 8013 | O | GLU | W | 197 | 32.284 | 30.974 | −55.961 | 1.00 | 33.07 | W | O |
| ATOM | 8014 | N | GLY | W | 198 | 33.893 | 30.072 | −54.671 | 1.00 | 23.59 | W | N |
| ATOM | 8015 | CA | GLY | W | 198 | 34.434 | 29.292 | −55.761 | 1.00 | 18.43 | W | C |
| ATOM | 8016 | C | GLY | W | 198 | 35.231 | 30.009 | −56.834 | 1.00 | 23.91 | W | C |
| ATOM | 8017 | O | GLY | W | 198 | 35.805 | 29.334 | −57.680 | 1.00 | 26.55 | W | O |
| ATOM | 8018 | N | GLN | W | 199 | 35.256 | 31.340 | −56.850 | 1.00 | 19.66 | W | N |
| ATOM | 8019 | CA | GLN | W | 199 | 36.024 | 32.033 | −57.893 | 1.00 | 24.73 | W | C |
| ATOM | 8020 | CB | GLN | W | 199 | 35.223 | 33.148 | −58.591 | 1.00 | 21.26 | W | C |
| ATOM | 8021 | CG | GLN | W | 199 | 33.725 | 32.986 | −58.611 | 1.00 | 31.42 | W | C |
| ATOM | 8022 | CD | GLN | W | 199 | 33.250 | 31.954 | −59.595 | 1.00 | 32.97 | W | C |
| ATOM | 8023 | OE1 | GLN | W | 199 | 33.492 | 32.065 | −60.795 | 1.00 | 39.68 | W | O |
| ATOM | 8024 | NE2 | GLN | W | 199 | 32.537 | 30.960 | −59.100 | 1.00 | 30.42 | W | N |
| ATOM | 8025 | C | GLN | W | 199 | 37.293 | 32.631 | −57.299 | 1.00 | 23.27 | W | C |
| ATOM | 8026 | O | GLN | W | 199 | 37.249 | 33.395 | −56.340 | 1.00 | 24.35 | W | O |
| ATOM | 8027 | N | SER | W | 200 | 38.426 | 32.294 | −57.893 | 1.00 | 25.72 | W | N |
| ATOM | 8028 | CA | SER | W | 200 | 39.703 | 32.800 | −57.421 | 1.00 | 24.29 | W | C |
| ATOM | 8029 | CB | SER | W | 200 | 40.809 | 31.775 | −57.708 | 1.00 | 20.09 | W | C |
| ATOM | 8030 | OG | SER | W | 200 | 42.089 | 32.350 | −57.447 | 1.00 | 30.25 | W | O |
| ATOM | 8031 | C | SER | W | 200 | 40.094 | 34.183 | −57.966 | 1.00 | 19.73 | W | C |
| ATOM | 8032 | O | SER | W | 200 | 40.085 | 34.396 | −59.178 | 1.00 | 22.24 | W | O |
| ATOM | 8033 | N | VAL | W | 201 | 40.355 | 35.127 | −57.054 | 1.00 | 20.80 | W | N |
| ATOM | 8034 | CA | VAL | W | 201 | 40.814 | 36.485 | −57.394 | 1.00 | 20.51 | W | C |
| ATOM | 8035 | CB | VAL | W | 201 | 40.119 | 37.597 | −56.569 | 1.00 | 21.82 | W | C |
| ATOM | 8036 | CG1 | VAL | W | 201 | 40.862 | 38.907 | −56.769 | 1.00 | 18.59 | W | C |
| ATOM | 8037 | CG2 | VAL | W | 201 | 38.665 | 37.778 | −57.013 | 1.00 | 15.29 | W | C |
| ATOM | 8038 | C | VAL | W | 201 | 42.315 | 36.499 | −57.069 | 1.00 | 27.17 | W | C |
| ATOM | 8039 | O | VAL | W | 201 | 42.720 | 36.179 | −55.944 | 1.00 | 31.06 | W | O |
| ATOM | 8040 | N | GLU | W | 202 | 43.119 | 36.959 | −58.019 | 1.00 | 26.13 | W | N |
| ATOM | 8041 | CA | GLU | W | 202 | 44.566 | 36.949 | −57.877 | 1.00 | 30.97 | W | C |
| ATOM | 8042 | CB | GLU | W | 202 | 45.086 | 35.870 | −58.857 | 1.00 | 30.15 | W | C |
| ATOM | 8043 | CG | GLU | W | 202 | 46.601 | 35.625 | −58.957 | 1.00 | 42.41 | W | C |
| ATOM | 8044 | CD | GLU | W | 202 | 46.955 | 34.406 | −59.848 | 1.00 | 44.59 | W | C |
| ATOM | 8045 | OE1 | GLU | W | 202 | 46.043 | 33.622 | −60.193 | 1.00 | 48.60 | W | O |
| ATOM | 8046 | OE2 | GLU | W | 202 | 48.145 | 34.212 | −60.191 | 1.00 | 43.90 | W | O |
| ATOM | 8047 | C | GLU | W | 202 | 45.292 | 38.287 | −58.100 | 1.00 | 29.26 | W | C |
| ATOM | 8048 | O | GLU | W | 202 | 44.847 | 39.133 | −58.873 | 1.00 | 28.95 | W | O |
| ATOM | 8049 | N | LYS | W | 203 | 46.402 | 38.465 | −57.383 | 1.00 | 28.34 | W | N |
| ATOM | 8050 | CA | LYS | W | 203 | 47.264 | 39.640 | −57.516 | 1.00 | 26.99 | W | C |
| ATOM | 8051 | CB | LYS | W | 203 | 47.108 | 40.594 | −56.333 | 1.00 | 25.06 | W | C |
| ATOM | 8052 | CG | LYS | W | 203 | 45.968 | 41.583 | −56.483 | 1.00 | 28.40 | W | C |
| ATOM | 8053 | CD | LYS | W | 203 | 46.138 | 42.433 | −57.722 | 1.00 | 33.73 | W | C |
| ATOM | 8054 | CE | LYS | W | 203 | 44.990 | 43.410 | −57.874 | 1.00 | 40.82 | W | C |
| ATOM | 8055 | NZ | LYS | W | 203 | 45.007 | 44.437 | −56.789 | 1.00 | 40.52 | W | N |
| ATOM | 8056 | C | LYS | W | 203 | 48.696 | 39.123 | −57.604 | 1.00 | 23.83 | W | C |
| ATOM | 8057 | O | LYS | W | 203 | 49.053 | 38.161 | −56.923 | 1.00 | 22.95 | W | O |
| ATOM | 8058 | N | SER | W | 204 | 49.505 | 39.740 | −58.461 | 1.00 | 22.63 | W | N |
| ATOM | 8059 | CA | SER | W | 204 | 50.891 | 39.307 | −58.641 | 1.00 | 25.42 | W | C |
| ATOM | 8060 | CB | SER | W | 204 | 51.038 | 38.568 | −59.971 | 1.00 | 20.43 | W | C |
| ATOM | 8061 | OG | SER | W | 204 | 50.185 | 37.438 | −60.021 | 1.00 | 25.21 | W | O |
| ATOM | 8062 | C | SER | W | 204 | 51.841 | 40.480 | −58.644 | 1.00 | 25.35 | W | C |
| ATOM | 8063 | O | SER | W | 204 | 51.413 | 41.627 | −58.730 | 1.00 | 30.90 | W | O |
| ATOM | 8064 | N | LEU | W | 205 | 53.134 | 40.184 | −58.566 | 1.00 | 27.06 | W | N |
| ATOM | 8065 | CA | LEU | W | 205 | 54.165 | 41.216 | −58.599 | 1.00 | 29.81 | W | C |
| ATOM | 8066 | CB | LEU | W | 205 | 54.218 | 41.981 | −57.258 | 1.00 | 32.70 | W | C |
| ATOM | 8067 | CG | LEU | W | 205 | 54.348 | 41.275 | −55.892 | 1.00 | 34.88 | W | C |
| ATOM | 8068 | CD1 | LEU | W | 205 | 55.487 | 40.294 | −55.873 | 1.00 | 29.74 | W | C |
| ATOM | 8069 | CD2 | LEU | W | 205 | 54.540 | 42.309 | −54.790 | 1.00 | 27.01 | W | C |
| ATOM | 8070 | C | LEU | W | 205 | 55.535 | 40.633 | −58.951 | 1.00 | 23.64 | W | C |
| ATOM | 8071 | O | LEU | W | 205 | 55.728 | 39.427 | −58.919 | 1.00 | 30.19 | W | O |
| ATOM | 8072 | N | SER | W | 206 | 56.481 | 41.505 | −59.282 | 1.00 | 38.33 | W | N |
| ATOM | 8073 | CA | SER | W | 206 | 57.850 | 41.100 | −59.617 | 1.00 | 40.52 | W | C |
| ATOM | 8074 | CB | SER | W | 206 | 58.099 | 41.276 | −61.113 | 1.00 | 39.36 | W | C |
| ATOM | 8075 | OG | SER | W | 206 | 57.253 | 40.426 | −61.860 | 1.00 | 51.08 | W | O |
| ATOM | 8076 | C | SER | W | 206 | 58.870 | 41.938 | −58.828 | 1.00 | 41.22 | W | C |
| ATOM | 8077 | O | SER | W | 206 | 59.934 | 41.448 | −58.416 | 1.00 | 45.61 | W | O |
| ATOM | 8078 | N | GLU | X | 1 | 17.024 | 39.706 | −14.636 | 1.00 | 50.17 | X | N |
| ATOM | 8079 | CA | GLU | X | 1 | 17.552 | 38.643 | −15.484 | 1.00 | 51.36 | X | C |
| ATOM | 8080 | CB | GLU | X | 1 | 17.709 | 39.127 | −16.941 | 1.00 | 58.85 | X | C |
| ATOM | 8081 | CG | GLU | X | 1 | 16.490 | 38.850 | −17.860 | 1.00 | 65.30 | X | C |
| ATOM | 8082 | CD | GLU | X | 1 | 15.604 | 40.077 | −18.102 | 1.00 | 68.29 | X | C |
| ATOM | 8083 | OE1 | GLU | X | 1 | 15.982 | 40.929 | −18.938 | 1.00 | 71.30 | X | O |
| ATOM | 8084 | OE2 | GLU | X | 1 | 14.522 | 40.185 | −17.480 | 1.00 | 69.64 | X | O |
| ATOM | 8085 | C | GLU | X | 1 | 18.877 | 38.070 | −14.983 | 1.00 | 43.46 | X | C |
| ATOM | 8086 | O | GLU | X | 1 | 19.586 | 38.700 | −14.214 | 1.00 | 40.00 | X | O |
| ATOM | 8087 | N | VAL | X | 2 | 19.191 | 36.862 | −15.436 | 1.00 | 34.94 | X | N |
| ATOM | 8088 | CA | VAL | X | 2 | 20.414 | 36.172 | −15.068 | 1.00 | 28.80 | X | C |
| ATOM | 8089 | CB | VAL | X | 2 | 20.359 | 34.693 | −15.522 | 1.00 | 27.53 | X | C |
| ATOM | 8090 | CG1 | VAL | X | 2 | 21.688 | 33.991 | −15.250 | 1.00 | 23.20 | X | C |
| ATOM | 8091 | CG2 | VAL | X | 2 | 19.219 | 33.960 | −14.821 | 1.00 | 20.71 | X | C |
| ATOM | 8092 | C | VAL | X | 2 | 21.626 | 36.830 | −15.730 | 1.00 | 37.99 | X | C |
| ATOM | 8093 | O | VAL | X | 2 | 21.558 | 37.246 | −16.889 | 1.00 | 38.96 | X | O |
| ATOM | 8094 | N | GLN | X | 3 | 22.723 | 36.937 | −14.980 | 1.00 | 38.61 | X | N |
| ATOM | 8095 | CA | GLN | X | 3 | 23.964 | 37.511 | −15.486 | 1.00 | 38.74 | X | C |
| ATOM | 8096 | CB | GLN | X | 3 | 24.071 | 39.006 | −15.159 | 1.00 | 44.35 | X | C |
| ATOM | 8097 | CG | GLN | X | 3 | 23.040 | 39.862 | −15.909 | 1.00 | 54.70 | X | C |
| ATOM | 8098 | CD | GLN | X | 3 | 23.137 | 41.361 | −15.625 | 1.00 | 56.58 | X | C |
| ATOM | 8099 | OE1 | GLN | X | 3 | 23.587 | 41.789 | −14.555 | 1.00 | 54.66 | X | O |
| ATOM | 8100 | NE2 | GLN | X | 3 | 22.695 | 42.166 | −16.589 | 1.00 | 56.28 | X | N |
| ATOM | 8101 | C | GLN | X | 3 | 25.145 | 36.760 | −14.905 | 1.00 | 36.86 | X | C |
| ATOM | 8102 | O | GLN | X | 3 | 25.084 | 36.261 | −13.784 | 1.00 | 38.56 | X | O |
| ATOM | 8103 | N | LEU | X | 4 | 26.192 | 36.632 | −15.708 | 1.00 | 26.85 | X | N |
| ATOM | 8104 | CA | LEU | X | 4 | 27.414 | 35.961 | −15.316 | 1.00 | 27.76 | X | C |
| ATOM | 8105 | CB | LEU | X | 4 | 27.570 | 34.642 | −16.083 | 1.00 | 26.25 | X | C |
| ATOM | 8106 | CG | LEU | X | 4 | 27.180 | 33.306 | −15.445 | 1.00 | 18.86 | X | C |
| ATOM | 8107 | CD1 | LEU | X | 4 | 25.708 | 33.235 | −15.188 | 1.00 | 27.14 | X | C |
| ATOM | 8108 | CD2 | LEU | X | 4 | 27.599 | 32.171 | −16.349 | 1.00 | 22.46 | X | C |
| ATOM | 8109 | C | LEU | X | 4 | 28.554 | 36.910 | −15.694 | 1.00 | 35.47 | X | C |
| ATOM | 8110 | O | LEU | X | 4 | 28.981 | 36.931 | −16.850 | 1.00 | 35.94 | X | O |
| ATOM | 8111 | N | GLN | X | 5 | 28.985 | 37.740 | −14.740 | 1.00 | 41.04 | X | N |
| ATOM | 8112 | CA | GLN | X | 5 | 30.064 | 38.715 | −14.956 | 1.00 | 40.59 | X | C |
| ATOM | 8113 | CB | GLN | X | 5 | 29.818 | 39.992 | −14.135 | 1.00 | 46.81 | X | C |
| ATOM | 8114 | CG | GLN | X | 5 | 28.537 | 40.750 | −14.498 | 1.00 | 58.40 | X | C |
| ATOM | 8115 | CD | GLN | X | 5 | 27.655 | 41.069 | −13.280 | 1.00 | 65.23 | X | C |
| ATOM | 8116 | OE1 | GLN | X | 5 | 27.428 | 42.240 | −12.950 | 1.00 | 67.57 | X | O |
| ATOM | 8117 | NE2 | GLN | X | 5 | 27.141 | 40.022 | −12.623 | 1.00 | 67.43 | X | N |
| ATOM | 8118 | C | GLN | X | 5 | 31.406 | 38.109 | −14.569 | 1.00 | 34.69 | X | C |
| ATOM | 8119 | O | GLN | X | 5 | 31.615 | 37.748 | −13.415 | 1.00 | 35.94 | X | O |
| ATOM | 8120 | N | GLN | X | 6 | 32.306 | 37.996 | −15.539 | 1.00 | 30.20 | X | N |
| ATOM | 8121 | CA | GLN | X | 6 | 33.633 | 37.429 | −15.311 | 1.00 | 31.05 | X | C |
| ATOM | 8122 | CB | GLN | X | 6 | 34.060 | 36.545 | −16.498 | 1.00 | 33.20 | X | C |
| ATOM | 8123 | CG | GLN | X | 6 | 33.108 | 35.383 | −16.820 | 1.00 | 29.52 | X | C |
| ATOM | 8124 | CD | GLN | X | 6 | 33.599 | 34.479 | −17.956 | 1.00 | 36.08 | X | C |
| ATOM | 8125 | OE1 | GLN | X | 6 | 32.802 | 33.996 | −18.771 | 1.00 | 41.15 | X | O |
| ATOM | 8126 | NE2 | GLN | X | 6 | 34.902 | 34.216 | −17.991 | 1.00 | 31.01 | X | N |
| ATOM | 8127 | C | GLN | X | 6 | 34.665 | 38.536 | −15.067 | 1.00 | 31.97 | X | C |
| ATOM | 8128 | O | GLN | X | 6 | 34.433 | 39.693 | −15.405 | 1.00 | 34.58 | X | O |
| ATOM | 8129 | N | SER | X | 7 | 35.799 | 38.176 | −14.477 | 1.00 | 29.82 | X | N |
| ATOM | 8130 | CA | SER | X | 7 | 36.840 | 39.143 | −14.183 | 1.00 | 32.54 | X | C |
| ATOM | 8131 | CB | SER | X | 7 | 37.747 | 38.629 | −13.060 | 1.00 | 32.88 | X | C |
| ATOM | 8132 | OG | SER | X | 7 | 38.192 | 37.310 | −13.317 | 1.00 | 37.32 | X | O |
| ATOM | 8133 | C | SER | X | 7 | 37.652 | 39.559 | −15.416 | 1.00 | 38.08 | X | C |
| ATOM | 8134 | O | SER | X | 7 | 37.660 | 38.860 | −16.436 | 1.00 | 38.53 | X | O |
| ATOM | 8135 | N | GLY | X | 8 | 38.351 | 40.689 | −15.288 | 1.00 | 37.21 | X | N |
| ATOM | 8136 | CA | GLY | X | 8 | 39.135 | 41.253 | −16.372 | 1.00 | 33.82 | X | C |
| ATOM | 8137 | C | GLY | X | 8 | 40.335 | 40.507 | −16.927 | 1.00 | 37.74 | X | C |
| ATOM | 8138 | O | GLY | X | 8 | 40.864 | 39.591 | −16.294 | 1.00 | 37.76 | X | O |
| ATOM | 8139 | N | PRO | X | 9 | 40.838 | 40.954 | −18.096 | 1.00 | 36.65 | X | N |
| ATOM | 8140 | CD | PRO | X | 9 | 40.384 | 42.172 | −18.793 | 1.00 | 39.05 | X | C |
| ATOM | 8141 | CA | PRO | X | 9 | 41.983 | 40.367 | −18.792 | 1.00 | 36.69 | X | C |
| ATOM | 8142 | CB | PRO | X | 9 | 42.251 | 41.360 | −19.928 | 1.00 | 33.24 | X | C |
| ATOM | 8143 | CG | PRO | X | 9 | 41.646 | 42.634 | −19.452 | 1.00 | 34.89 | X | C |
| ATOM | 8144 | C | PRO | X | 9 | 43.199 | 40.138 | −17.910 | 1.00 | 40.16 | X | C |
| ATOM | 8145 | O | PRO | X | 9 | 43.359 | 40.766 | −16.862 | 1.00 | 41.20 | X | O |
| ATOM | 8146 | N | GLU | X | 10 | 44.048 | 39.226 | −18.365 | 1.00 | 37.19 | X | N |
| ATOM | 8147 | CA | GLU | X | 10 | 45.237 | 38.845 | −17.641 | 1.00 | 43.75 | X | C |
| ATOM | 8148 | CB | GLU | X | 10 | 44.994 | 37.467 | −17.008 | 1.00 | 45.65 | X | C |
| ATOM | 8149 | CG | GLU | X | 10 | 45.837 | 37.142 | −15.786 | 1.00 | 55.79 | X | C |
| ATOM | 8150 | CD | GLU | X | 10 | 45.198 | 37.585 | −14.468 | 1.00 | 57.16 | X | C |
| ATOM | 8151 | OE1 | GLU | X | 10 | 44.381 | 38.536 | −14.470 | 1.00 | 57.16 | X | O |
| ATOM | 8152 | OE2 | GLU | X | 10 | 45.519 | 36.970 | −13.425 | 1.00 | 60.75 | X | O |
| ATOM | 8153 | C | GLU | X | 10 | 46.406 | 38.759 | −18.615 | 1.00 | 44.64 | X | C |
| ATOM | 8154 | O | GLU | X | 10 | 46.218 | 38.421 | −19.795 | 1.00 | 49.42 | X | O |
| ATOM | 8155 | N | LEU | X | 11 | 47.597 | 39.113 | −18.129 | 1.00 | 40.60 | X | N |
| ATOM | 8156 | CA | LEU | X | 11 | 48.836 | 39.046 | −18.913 | 1.00 | 36.93 | X | C |
| ATOM | 8157 | CB | LEU | X | 11 | 49.181 | 40.398 | −19.520 | 1.00 | 37.97 | X | C |
| ATOM | 8158 | CG | LEU | X | 11 | 50.577 | 40.538 | −20.141 | 1.00 | 39.06 | X | C |
| ATOM | 8159 | CD1 | LEU | X | 11 | 50.900 | 39.396 | −21.105 | 1.00 | 33.47 | X | C |
| ATOM | 8160 | CD2 | LEU | X | 11 | 50.649 | 41.895 | −20.828 | 1.00 | 38.16 | X | C |
| ATOM | 8161 | C | LEU | X | 11 | 49.923 | 38.611 | −17.954 | 1.00 | 35.65 | X | C |
| ATOM | 8162 | O | LEU | X | 11 | 50.260 | 39.343 | −17.026 | 1.00 | 33.34 | X | O |
| ATOM | 8163 | N | VAL | X | 12 | 50.456 | 37.411 | −18.175 | 1.00 | 35.57 | X | N |
| ATOM | 8164 | CA | VAL | X | 12 | 51.467 | 36.840 | −17.291 | 1.00 | 39.50 | X | C |
| ATOM | 8165 | CB | VAL | X | 12 | 50.796 | 35.937 | −16.210 | 1.00 | 43.39 | X | C |
| ATOM | 8166 | CG1 | VAL | X | 12 | 49.906 | 36.764 | −15.264 | 1.00 | 38.34 | X | C |
| ATOM | 8167 | CG2 | VAL | X | 12 | 49.984 | 34.850 | −16.891 | 1.00 | 32.63 | X | C |
| ATOM | 8168 | C | VAL | X | 12 | 52.495 | 35.979 | −18.030 | 1.00 | 43.38 | X | C |
| ATOM | 8169 | O | VAL | X | 12 | 52.264 | 35.559 | −19.166 | 1.00 | 48.04 | X | O |
| ATOM | 8170 | N | LYS | X | 13 | 53.600 | 35.671 | −17.351 | 1.00 | 40.06 | X | N |
| ATOM | 8171 | CA | LYS | X | 13 | 54.663 | 34.857 | −17.931 | 1.00 | 42.05 | X | C |
| ATOM | 8172 | CB | LYS | X | 13 | 56.038 | 35.306 | −17.395 | 1.00 | 45.87 | X | C |
| ATOM | 8173 | CG | LYS | X | 13 | 56.018 | 35.944 | −15.996 | 1.00 | 52.45 | X | C |
| ATOM | 8174 | CD | LYS | X | 13 | 55.614 | 34.942 | −14.883 | 1.00 | 58.17 | X | C |
| ATOM | 8175 | CE | LYS | X | 13 | 55.663 | 35.543 | −13.458 | 1.00 | 52.81 | X | C |
| ATOM | 8176 | NZ | LYS | X | 13 | 57.047 | 35.874 | −12.995 | 1.00 | 52.25 | X | N |
| ATOM | 8177 | C | LYS | X | 13 | 54.433 | 33.371 | −17.682 | 1.00 | 39.82 | X | C |
| ATOM | 8178 | O | LYS | X | 13 | 53.591 | 32.998 | −16.863 | 1.00 | 39.32 | X | O |
| ATOM | 8179 | N | PRO | X | 14 | 55.129 | 32.500 | −18.436 | 1.00 | 38.46 | X | N |
| ATOM | 8180 | CD | PRO | X | 14 | 55.984 | 32.795 | −19.595 | 1.00 | 38.14 | X | C |
| ATOM | 8181 | CA | PRO | X | 14 | 54.982 | 31.051 | −18.272 | 1.00 | 41.01 | X | C |
| ATOM | 8182 | CB | PRO | X | 14 | 55.984 | 30.489 | −19.276 | 1.00 | 38.08 | X | C |
| ATOM | 8183 | CG | PRO | X | 14 | 55.946 | 31.489 | −20.354 | 1.00 | 39.32 | X | C |
| ATOM | 8184 | C | PRO | X | 14 | 55.355 | 30.659 | −16.866 | 1.00 | 45.33 | X | C |
| ATOM | 8185 | O | PRO | X | 14 | 56.207 | 31.307 | −16.245 | 1.00 | 49.62 | X | O |
| ATOM | 8186 | N | GLY | X | 15 | 54.713 | 29.606 | −16.366 | 1.00 | 43.96 | X | N |
| ATOM | 8187 | CA | GLY | X | 15 | 54.988 | 29.147 | −15.015 | 1.00 | 46.69 | X | C |
| ATOM | 8188 | C | GLY | X | 15 | 54.103 | 29.803 | −13.966 | 1.00 | 40.04 | X | C |
| ATOM | 8189 | O | GLY | X | 15 | 53.941 | 29.269 | −12.868 | 1.00 | 44.19 | X | O |
| ATOM | 8190 | N | ALA | X | 16 | 53.549 | 30.968 | −14.293 | 1.00 | 39.19 | X | N |
| ATOM | 8191 | CA | ALA | X | 16 | 52.657 | 31.683 | −13.384 | 1.00 | 35.59 | X | C |
| ATOM | 8192 | CB | ALA | X | 16 | 52.386 | 33.071 | −13.920 | 1.00 | 31.71 | X | C |
| ATOM | 8193 | C | ALA | X | 16 | 51.332 | 30.929 | −13.199 | 1.00 | 37.69 | X | C |
| ATOM | 8194 | O | ALA | X | 16 | 51.153 | 29.794 | −13.686 | 1.00 | 34.49 | X | O |
| ATOM | 8195 | N | SER | X | 17 | 50.410 | 31.567 | −12.485 | 1.00 | 35.48 | X | N |
| ATOM | 8196 | CA | SER | X | 17 | 49.100 | 30.987 | −12.231 | 1.00 | 35.17 | X | C |
| ATOM | 8197 | CB | SER | X | 17 | 49.073 | 30.248 | −10.893 | 1.00 | 39.22 | X | C |
| ATOM | 8198 | OG | SER | X | 17 | 48.457 | 31.047 | −9.896 | 1.00 | 43.29 | X | O |
| ATOM | 8199 | C | SER | X | 17 | 48.057 | 32.094 | −12.245 | 1.00 | 37.41 | X | C |
| ATOM | 8200 | O | SER | X | 17 | 48.368 | 33.263 | −11.979 | 1.00 | 36.73 | X | O |
| ATOM | 8201 | N | VAL | X | 18 | 46.816 | 31.721 | −12.549 | 1.00 | 32.25 | X | N |
| ATOM | 8202 | CA | VAL | X | 18 | 45.748 | 32.698 | −12.618 | 1.00 | 28.76 | X | C |
| ATOM | 8203 | CB | VAL | X | 18 | 45.611 | 33.254 | −14.074 | 1.00 | 31.33 | X | C |
| ATOM | 8204 | CG1 | VAL | X | 18 | 45.428 | 32.130 | −15.071 | 1.00 | 31.43 | X | C |
| ATOM | 8205 | CG2 | VAL | X | 18 | 44.464 | 34.211 | −14.180 | 1.00 | 33.81 | X | C |
| ATOM | 8206 | C | VAL | X | 18 | 44.450 | 32.110 | −12.081 | 1.00 | 33.66 | X | C |
| ATOM | 8207 | O | VAL | X | 18 | 44.198 | 30.904 | −12.179 | 1.00 | 33.41 | X | O |
| ATOM | 8208 | N | LYS | X | 19 | 43.670 | 32.953 | −11.422 | 1.00 | 32.35 | X | N |
| ATOM | 8209 | CA | LYS | X | 19 | 42.412 | 32.518 | −10.852 | 1.00 | 38.66 | X | C |
| ATOM | 8210 | CB | LYS | X | 19 | 42.492 | 32.494 | −9.316 | 1.00 | 43.39 | X | C |
| ATOM | 8211 | CG | LYS | X | 19 | 41.240 | 31.927 | −8.618 | 1.00 | 49.16 | X | C |
| ATOM | 8212 | CD | LYS | X | 19 | 41.430 | 31.810 | −7.111 | 1.00 | 48.43 | X | C |
| ATOM | 8213 | CE | LYS | X | 19 | 40.182 | 31.283 | −6.432 | 1.00 | 49.77 | X | C |
| ATOM | 8214 | NZ | LYS | X | 19 | 40.419 | 31.060 | −4.977 | 1.00 | 53.70 | X | N |
| ATOM | 8215 | C | LYS | X | 19 | 41.322 | 33.465 | −11.322 | 1.00 | 38.45 | X | C |
| ATOM | 8216 | O | LYS | X | 19 | 41.265 | 34.626 | −10.889 | 1.00 | 32.74 | X | O |
| ATOM | 8217 | N | ILE | X | 20 | 40.481 | 32.958 | −12.229 | 1.00 | 33.64 | X | N |
| ATOM | 8218 | CA | ILE | X | 20 | 39.382 | 33.721 | −12.811 | 1.00 | 25.21 | X | C |
| ATOM | 8219 | CB | ILE | X | 20 | 39.147 | 33.299 | −14.266 | 1.00 | 23.90 | X | C |
| ATOM | 8220 | CG2 | ILE | X | 20 | 38.093 | 34.170 | −14.892 | 1.00 | 23.56 | X | C |
| ATOM | 8221 | CG1 | ILE | X | 20 | 40.447 | 33.430 | −15.053 | 1.00 | 27.06 | X | C |
| ATOM | 8222 | CD1 | ILE | X | 20 | 40.527 | 32.527 | −16.272 | 1.00 | 26.77 | X | C |
| ATOM | 8223 | C | ILE | X | 20 | 38.112 | 33.520 | −11.993 | 1.00 | 22.78 | X | C |
| ATOM | 8224 | O | ILE | X | 20 | 37.878 | 32.459 | −11.441 | 1.00 | 23.61 | X | O |
| ATOM | 8225 | N | SER | X | 21 | 37.302 | 34.560 | −11.911 | 1.00 | 26.22 | X | N |
| ATOM | 8226 | CA | SER | X | 21 | 36.073 | 34.503 | −11.159 | 1.00 | 28.29 | X | C |
| ATOM | 8227 | CB | SER | X | 21 | 36.096 | 35.555 | −10.048 | 1.00 | 30.44 | X | C |
| ATOM | 8228 | OG | SER | X | 21 | 35.907 | 36.855 | −10.568 | 1.00 | 31.72 | X | O |
| ATOM | 8229 | C | SER | X | 21 | 34.901 | 34.762 | −12.097 | 1.00 | 33.04 | X | C |
| ATOM | 8230 | O | SER | X | 21 | 35.080 | 35.373 | −13.154 | 1.00 | 30.47 | X | O |
| ATOM | 8231 | N | CYS | X | 22 | 33.710 | 34.318 | −11.683 | 1.00 | 29.91 | X | N |
| ATOM | 8232 | CA | CYS | X | 22 | 32.474 | 34.469 | −12.448 | 1.00 | 29.71 | X | C |
| ATOM | 8233 | C | CYS | X | 22 | 31.394 | 34.748 | −11.419 | 1.00 | 32.17 | X | C |
| ATOM | 8234 | O | CYS | X | 22 | 31.036 | 33.882 | −10.611 | 1.00 | 30.33 | X | O |
| ATOM | 8235 | CB | CYS | X | 22 | 32.163 | 33.164 | −13.204 | 1.00 | 38.35 | X | C |
| ATOM | 8236 | SG | CYS | X | 22 | 30.668 | 33.114 | −14.267 | 1.00 | 45.81 | X | S |
| ATOM | 8237 | N | LYS | X | 23 | 30.919 | 35.982 | −11.408 | 1.00 | 28.58 | X | N |
| ATOM | 8238 | CA | LYS | X | 23 | 29.902 | 36.374 | −10.469 | 1.00 | 34.15 | X | C |
| ATOM | 8239 | CB | LYS | X | 23 | 30.048 | 37.855 | −10.125 | 1.00 | 36.18 | X | C |
| ATOM | 8240 | CG | LYS | X | 23 | 29.166 | 38.310 | −8.974 | 1.00 | 41.79 | X | C |
| ATOM | 8241 | CD | LYS | X | 23 | 29.794 | 37.936 | −7.639 | 1.00 | 54.09 | X | C |
| ATOM | 8242 | CE | LYS | X | 23 | 31.119 | 38.701 | −7.416 | 1.00 | 60.78 | X | C |
| ATOM | 8243 | NZ | LYS | X | 23 | 31.968 | 38.152 | −6.307 | 1.00 | 58.98 | X | N |
| ATOM | 8244 | C | LYS | X | 23 | 28.528 | 36.109 | −11.057 | 1.00 | 34.14 | X | C |
| ATOM | 8245 | O | LYS | X | 23 | 28.052 | 36.878 | −11.894 | 1.00 | 36.49 | X | O |
| ATOM | 8246 | N | ALA | X | 24 | 27.890 | 35.032 | −10.601 | 1.00 | 35.33 | X | N |
| ATOM | 8247 | CA | ALA | X | 24 | 26.556 | 34.657 | −11.071 | 1.00 | 36.10 | X | C |
| ATOM | 8248 | CB | ALA | X | 24 | 26.380 | 33.147 | −11.020 | 1.00 | 32.70 | X | C |
| ATOM | 8249 | C | ALA | X | 24 | 25.477 | 35.326 | −10.237 | 1.00 | 39.92 | X | C |
| ATOM | 8250 | O | ALA | X | 24 | 25.437 | 35.153 | −9.021 | 1.00 | 49.13 | X | O |
| ATOM | 8251 | N | SER | X | 25 | 24.584 | 36.064 | −10.891 | 1.00 | 37.95 | X | N |
| ATOM | 8252 | CA | SER | X | 25 | 23.503 | 36.735 | −10.187 | 1.00 | 38.36 | X | C |
| ATOM | 8253 | CB | SER | X | 25 | 23.880 | 38.184 | −9.896 | 1.00 | 38.41 | X | C |
| ATOM | 8254 | OG | SER | X | 25 | 24.082 | 38.900 | −11.096 | 1.00 | 45.31 | X | O |
| ATOM | 8255 | C | SER | X | 25 | 22.210 | 36.691 | −10.992 | 1.00 | 38.33 | X | C |
| ATOM | 8256 | O | SER | X | 25 | 22.234 | 36.435 | −12.196 | 1.00 | 44.13 | X | O |
| ATOM | 8257 | N | GLY | X | 26 | 21.084 | 36.930 | −10.321 | 1.00 | 32.61 | X | N |
| ATOM | 8258 | CA | GLY | X | 26 | 19.801 | 36.932 | −11.000 | 1.00 | 26.62 | X | C |
| ATOM | 8259 | C | GLY | X | 26 | 19.043 | 35.621 | −10.929 | 1.00 | 33.07 | X | C |
| ATOM | 8260 | O | GLY | X | 26 | 18.005 | 35.461 | −11.574 | 1.00 | 31.43 | X | O |
| ATOM | 8261 | N | TYR | X | 27 | 19.558 | 34.678 | −10.146 | 1.00 | 31.67 | X | N |
| ATOM | 8262 | CA | TYR | X | 27 | 18.909 | 33.389 | −9.991 | 1.00 | 31.22 | X | C |
| ATOM | 8263 | CB | TYR | X | 27 | 19.073 | 32.566 | −11.272 | 1.00 | 20.35 | X | C |
| ATOM | 8264 | CG | TYR | X | 27 | 20.459 | 32.027 | −11.482 | 1.00 | 15.08 | X | C |
| ATOM | 8265 | CD1 | TYR | X | 27 | 21.468 | 32.831 | −12.013 | 1.00 | 15.11 | X | C |
| ATOM | 8266 | CE1 | TYR | X | 27 | 22.761 | 32.344 | −12.200 | 1.00 | 12.18 | X | C |
| ATOM | 8267 | CD2 | TYR | X | 27 | 20.775 | 30.719 | −11.142 | 1.00 | 9.72 | X | C |
| ATOM | 8268 | CE2 | TYR | X | 27 | 22.083 | 30.220 | −11.329 | 1.00 | 12.67 | X | C |
| ATOM | 8269 | CZ | TYR | X | 27 | 23.062 | 31.047 | −11.857 | 1.00 | 13.69 | X | C |
| ATOM | 8270 | OH | TYR | X | 27 | 24.354 | 30.588 | −12.034 | 1.00 | 23.06 | X | O |
| ATOM | 8271 | C | TYR | X | 27 | 19.476 | 32.622 | −8.790 | 1.00 | 36.08 | X | C |
| ATOM | 8272 | O | TYR | X | 27 | 20.426 | 33.077 | −8.144 | 1.00 | 33.75 | X | O |
| ATOM | 8273 | N | SER | X | 28 | 18.878 | 31.471 | −8.489 | 1.00 | 33.39 | X | N |
| ATOM | 8274 | CA | SER | X | 28 | 19.341 | 30.642 | −7.386 | 1.00 | 33.54 | X | C |
| ATOM | 8275 | CB | SER | X | 28 | 18.251 | 29.673 | −6.949 | 1.00 | 33.12 | X | C |
| ATOM | 8276 | OG | SER | X | 28 | 18.716 | 28.867 | −5.889 | 1.00 | 46.90 | X | O |
| ATOM | 8277 | C | SER | X | 28 | 20.615 | 29.900 | −7.792 | 1.00 | 33.07 | X | C |
| ATOM | 8278 | O | SER | X | 28 | 20.599 | 28.967 | −8.597 | 1.00 | 35.86 | X | O |
| ATOM | 8279 | N | PHE | X | 29 | 21.710 | 30.310 | −7.169 | 1.00 | 31.51 | X | N |
| ATOM | 8280 | CA | PHE | X | 29 | 23.044 | 29.805 | −7.428 | 1.00 | 27.85 | X | C |
| ATOM | 8281 | CB | PHE | X | 29 | 24.024 | 30.507 | −6.480 | 1.00 | 24.83 | X | C |
| ATOM | 8282 | CG | PHE | X | 29 | 25.465 | 30.269 | −6.800 | 1.00 | 27.41 | X | C |
| ATOM | 8283 | CD1 | PHE | X | 29 | 26.016 | 30.734 | −7.991 | 1.00 | 27.68 | X | C |
| ATOM | 8284 | CD2 | PHE | X | 29 | 26.282 | 29.586 | −5.913 | 1.00 | 25.26 | X | C |
| ATOM | 8285 | CE1 | PHE | X | 29 | 27.367 | 30.521 | −8.293 | 1.00 | 18.36 | X | C |
| ATOM | 8286 | CE2 | PHE | X | 29 | 27.628 | 29.371 | −6.215 | 1.00 | 24.31 | X | C |
| ATOM | 8287 | CZ | PHE | X | 29 | 28.162 | 29.842 | −7.404 | 1.00 | 22.12 | X | C |
| ATOM | 8288 | C | PHE | X | 29 | 23.272 | 28.304 | −7.407 | 1.00 | 28.82 | X | C |
| ATOM | 8289 | O | PHE | X | 29 | 23.952 | 27.765 | −8.280 | 1.00 | 36.08 | X | O |
| ATOM | 8290 | N | THR | X | 30 | 22.715 | 27.631 | −6.416 | 1.00 | 30.18 | X | N |
| ATOM | 8291 | CA | THR | X | 30 | 22.914 | 26.194 | −6.264 | 1.00 | 27.61 | X | C |
| ATOM | 8292 | CB | THR | X | 30 | 22.631 | 25.770 | −4.816 | 1.00 | 32.74 | X | C |
| ATOM | 8293 | OG1 | THR | X | 30 | 21.229 | 25.931 | −4.549 | 1.00 | 37.80 | X | O |
| ATOM | 8294 | CG2 | THR | X | 30 | 23.431 | 26.623 | −3.832 | 1.00 | 32.10 | X | C |
| ATOM | 8295 | C | THR | X | 30 | 22.052 | 25.307 | −7.165 | 1.00 | 28.96 | X | C |
| ATOM | 8296 | O | THR | X | 30 | 22.166 | 24.082 | −7.100 | 1.00 | 26.54 | X | O |
| ATOM | 8297 | N | GLY | X | 31 | 21.200 | 25.917 | −7.989 | 1.00 | 24.05 | X | N |
| ATOM | 8298 | CA | GLY | X | 31 | 20.314 | 25.153 | −8.855 | 1.00 | 26.88 | X | C |
| ATOM | 8299 | C | GLY | X | 31 | 20.626 | 25.026 | −10.341 | 1.00 | 33.06 | X | C |
| ATOM | 8300 | O | GLY | X | 31 | 19.736 | 24.738 | −11.132 | 1.00 | 35.66 | X | O |
| ATOM | 8301 | N | HIS | X | 32 | 21.872 | 25.256 | −10.736 | 1.00 | 30.46 | X | N |
| ATOM | 8302 | CA | HIS | X | 32 | 22.280 | 25.129 | −12.133 | 1.00 | 25.42 | X | C |
| ATOM | 8303 | CB | HIS | X | 32 | 22.080 | 26.441 | −12.898 | 1.00 | 23.35 | X | C |
| ATOM | 8304 | CG | HIS | X | 32 | 20.684 | 26.665 | −13.393 | 1.00 | 27.33 | X | C |
| ATOM | 8305 | CD2 | HIS | X | 32 | 20.145 | 26.527 | −14.629 | 1.00 | 27.71 | X | C |
| ATOM | 8306 | ND1 | HIS | X | 32 | 19.667 | 27.124 | −12.583 | 1.00 | 29.36 | X | N |
| ATOM | 8307 | CE1 | HIS | X | 32 | 18.563 | 27.262 | −13.299 | 1.00 | 21.57 | X | C |
| ATOM | 8308 | NE2 | HIS | X | 32 | 18.827 | 26.905 | −14.542 | 1.00 | 24.78 | X | N |
| ATOM | 8309 | C | HIS | X | 32 | 23.759 | 24.794 | −12.108 | 1.00 | 27.81 | X | C |
| ATOM | 8310 | O | HIS | X | 32 | 24.463 | 25.177 | −11.180 | 1.00 | 29.30 | X | O |
| ATOM | 8311 | N | LEU | X | 33 | 24.225 | 24.033 | −13.090 | 1.00 | 24.29 | X | N |
| ATOM | 8312 | CA | LEU | X | 33 | 25.634 | 23.709 | −13.149 | 1.00 | 23.99 | X | C |
| ATOM | 8313 | CB | LEU | X | 33 | 25.881 | 22.501 | −14.049 | 1.00 | 20.43 | X | C |
| ATOM | 8314 | CG | LEU | X | 33 | 25.447 | 21.124 | −13.569 | 1.00 | 24.28 | X | C |
| ATOM | 8315 | CD1 | LEU | X | 33 | 25.943 | 20.072 | −14.557 | 1.00 | 22.59 | X | C |
| ATOM | 8316 | CD2 | LEU | X | 33 | 26.046 | 20.860 | −12.190 | 1.00 | 23.99 | X | C |
| ATOM | 8317 | C | LEU | X | 33 | 26.380 | 24.913 | −13.721 | 1.00 | 24.71 | X | C |
| ATOM | 8318 | O | LEU | X | 33 | 25.776 | 25.806 | −14.327 | 1.00 | 20.95 | X | O |
| ATOM | 8319 | N | LEU | X | 34 | 27.678 | 24.975 | −13.454 | 1.00 | 20.12 | X | N |
| ATOM | 8320 | CA | LEU | X | 34 | 28.507 | 26.038 | −14.003 | 1.00 | 18.65 | X | C |
| ATOM | 8321 | CB | LEU | X | 34 | 28.969 | 27.004 | −12.937 | 1.00 | 20.40 | X | C |
| ATOM | 8322 | CG | LEU | X | 34 | 27.921 | 28.076 | −12.711 | 1.00 | 22.75 | X | C |
| ATOM | 8323 | CD1 | LEU | X | 34 | 28.296 | 28.906 | −11.497 | 1.00 | 25.49 | X | C |
| ATOM | 8324 | CD2 | LEU | X | 34 | 27.825 | 28.937 | −13.954 | 1.00 | 21.35 | X | C |
| ATOM | 8325 | C | LEU | X | 34 | 29.675 | 25.344 | −14.646 | 1.00 | 20.74 | X | C |
| ATOM | 8326 | O | LEU | X | 34 | 30.346 | 24.512 | −14.017 | 1.00 | 22.75 | X | O |
| ATOM | 8327 | N | ASN | X | 35 | 29.825 | 25.593 | −15.941 | 1.00 | 22.01 | X | N |
| ATOM | 8328 | CA | ASN | X | 35 | 30.886 | 24.989 | −16.729 | 1.00 | 23.29 | X | C |
| ATOM | 8329 | CB | ASN | X | 35 | 30.327 | 24.497 | −18.076 | 1.00 | 22.10 | X | C |
| ATOM | 8330 | CG | ASN | X | 35 | 29.347 | 23.361 | −17.937 | 1.00 | 25.44 | X | C |
| ATOM | 8331 | OD1 | ASN | X | 35 | 29.737 | 22.192 | −17.896 | 1.00 | 24.26 | X | O |
| ATOM | 8332 | ND2 | ASN | X | 35 | 28.060 | 23.687 | −17.948 | 1.00 | 20.39 | X | N |
| ATOM | 8333 | C | ASN | X | 35 | 31.943 | 26.038 | −17.032 | 1.00 | 24.40 | X | C |
| ATOM | 8334 | O | ASN | X | 35 | 31.666 | 27.242 | −16.994 | 1.00 | 14.24 | X | O |
| ATOM | 8335 | N | TRP | X | 36 | 33.134 | 25.562 | −17.388 | 1.00 | 21.67 | X | N |
| ATOM | 8336 | CA | TRP | X | 36 | 34.236 | 26.432 | −17.776 | 1.00 | 22.81 | X | C |
| ATOM | 8337 | CB | TRP | X | 36 | 35.393 | 26.384 | −16.764 | 1.00 | 27.52 | X | C |
| ATOM | 8338 | CG | TRP | X | 36 | 35.154 | 27.274 | −15.586 | 1.00 | 25.71 | X | C |
| ATOM | 8339 | CD2 | TRP | X | 36 | 35.294 | 28.702 | −15.544 | 1.00 | 29.02 | X | C |
| ATOM | 8340 | CE2 | TRP | X | 36 | 34.842 | 29.127 | −14.284 | 1.00 | 27.51 | X | C |
| ATOM | 8341 | CE3 | TRP | X | 36 | 35.756 | 29.663 | −16.456 | 1.00 | 31.03 | X | C |
| ATOM | 8342 | CD1 | TRP | X | 36 | 34.658 | 26.907 | −14.380 | 1.00 | 31.60 | X | C |
| ATOM | 8343 | NE1 | TRP | X | 36 | 34.459 | 28.013 | −13.592 | 1.00 | 27.30 | X | N |
| ATOM | 8344 | CZ2 | TRP | X | 36 | 34.831 | 30.474 | −13.905 | 1.00 | 31.14 | X | C |
| ATOM | 8345 | CZ3 | TRP | X | 36 | 35.747 | 31.006 | −16.077 | 1.00 | 27.65 | X | C |
| ATOM | 8346 | CH2 | TRP | X | 36 | 35.286 | 31.394 | −14.811 | 1.00 | 31.33 | X | C |
| ATOM | 8347 | C | TRP | X | 36 | 34.685 | 25.959 | −19.142 | 1.00 | 21.54 | X | C |
| ATOM | 8348 | O | TRP | X | 36 | 34.983 | 24.771 | −19.338 | 1.00 | 22.90 | X | O |
| ATOM | 8349 | N | VAL | X | 37 | 34.688 | 26.894 | −20.090 | 1.00 | 20.85 | X | N |
| ATOM | 8350 | CA | VAL | X | 37 | 35.058 | 26.625 | −21.478 | 1.00 | 21.03 | X | C |
| ATOM | 8351 | CB | VAL | X | 37 | 33.861 | 26.917 | −22.423 | 1.00 | 22.33 | X | C |
| ATOM | 8352 | CG1 | VAL | X | 37 | 34.292 | 26.807 | −23.898 | 1.00 | 23.65 | X | C |
| ATOM | 8353 | CG2 | VAL | X | 37 | 32.709 | 25.952 | −22.129 | 1.00 | 21.19 | X | C |
| ATOM | 8354 | C | VAL | X | 37 | 36.251 | 27.464 | −21.939 | 1.00 | 21.31 | X | C |
| ATOM | 8355 | O | VAL | X | 37 | 36.331 | 28.655 | −21.668 | 1.00 | 24.44 | X | O |
| ATOM | 8356 | N | LYS | X | 38 | 37.114 | 26.838 | −22.724 | 1.00 | 21.13 | X | N |
| ATOM | 8357 | CA | LYS | X | 38 | 38.306 | 27.470 | −23.263 | 1.00 | 28.66 | X | C |
| ATOM | 8358 | CB | LYS | X | 38 | 39.517 | 26.579 | −22.951 | 1.00 | 28.07 | X | C |
| ATOM | 8359 | CG | LYS | X | 38 | 40.859 | 27.075 | −23.449 | 1.00 | 25.96 | X | C |
| ATOM | 8360 | CD | LYS | X | 38 | 41.864 | 25.947 | −23.321 | 1.00 | 33.56 | X | C |
| ATOM | 8361 | CE | LYS | X | 38 | 43.257 | 26.331 | −23.788 | 1.00 | 36.94 | X | C |
| ATOM | 8362 | NZ | LYS | X | 38 | 44.151 | 25.130 | −23.711 | 1.00 | 44.85 | X | N |
| ATOM | 8363 | C | LYS | X | 38 | 38.213 | 27.690 | −24.781 | 1.00 | 30.42 | X | C |
| ATOM | 8364 | O | LYS | X | 38 | 38.075 | 26.735 | −25.545 | 1.00 | 27.52 | X | O |
| ATOM | 8365 | N | GLN | X | 39 | 38.349 | 28.947 | −25.202 | 1.00 | 35.51 | X | N |
| ATOM | 8366 | CA | GLN | X | 39 | 38.318 | 29.335 | −26.616 | 1.00 | 36.50 | X | C |
| ATOM | 8367 | CB | GLN | X | 39 | 37.333 | 30.487 | −26.843 | 1.00 | 34.30 | X | C |
| ATOM | 8368 | CG | GLN | X | 39 | 37.168 | 30.880 | −28.298 | 1.00 | 28.71 | X | C |
| ATOM | 8369 | CD | GLN | X | 39 | 36.188 | 32.026 | −28.478 | 1.00 | 36.76 | X | C |
| ATOM | 8370 | OE1 | GLN | X | 39 | 36.259 | 33.045 | −27.773 | 1.00 | 37.30 | X | O |
| ATOM | 8371 | NE2 | GLN | X | 39 | 35.265 | 31.871 | −29.429 | 1.00 | 37.79 | X | N |
| ATOM | 8372 | C | GLN | X | 39 | 39.707 | 29.768 | −27.084 | 1.00 | 39.03 | X | C |
| ATOM | 8373 | O | GLN | X | 39 | 40.220 | 30.814 | −26.682 | 1.00 | 40.32 | X | O |
| ATOM | 8374 | N | SER | X | 40 | 40.293 | 28.959 | −27.959 | 1.00 | 43.46 | X | N |
| ATOM | 8375 | CA | SER | X | 40 | 41.621 | 29.204 | −28.515 | 1.00 | 40.84 | X | C |
| ATOM | 8376 | CB | SER | X | 40 | 42.358 | 27.875 | −28.679 | 1.00 | 35.48 | X | C |
| ATOM | 8377 | OG | SER | X | 40 | 42.479 | 27.194 | −27.449 | 1.00 | 35.33 | X | O |
| ATOM | 8378 | C | SER | X | 40 | 41.571 | 29.889 | −29.883 | 1.00 | 47.58 | X | C |
| ATOM | 8379 | O | SER | X | 40 | 40.867 | 29.433 | −30.779 | 1.00 | 51.69 | X | O |
| ATOM | 8380 | N | HIS | X | 41 | 42.355 | 30.953 | −30.050 | 1.00 | 52.42 | X | N |
| ATOM | 8381 | CA | HIS | X | 41 | 42.450 | 31.691 | −31.316 | 1.00 | 55.91 | X | C |
| ATOM | 8382 | CB | HIS | X | 41 | 42.829 | 30.753 | −32.468 | 1.00 | 52.22 | X | C |
| ATOM | 8383 | CG | HIS | X | 41 | 43.971 | 29.844 | −32.154 | 1.00 | 53.79 | X | C |
| ATOM | 8384 | CD2 | HIS | X | 41 | 45.245 | 30.107 | −31.776 | 1.00 | 54.76 | X | C |
| ATOM | 8385 | ND1 | HIS | X | 41 | 43.860 | 28.471 | −32.204 | 1.00 | 57.35 | X | N |
| ATOM | 8386 | CE1 | HIS | X | 41 | 45.016 | 27.926 | −31.868 | 1.00 | 59.32 | X | C |
| ATOM | 8387 | NE2 | HIS | X | 41 | 45.874 | 28.897 | −31.604 | 1.00 | 58.48 | X | N |
| ATOM | 8388 | C | HIS | X | 41 | 41.240 | 32.520 | −31.744 | 1.00 | 59.68 | X | C |
| ATOM | 8389 | O | HIS | X | 41 | 41.335 | 33.300 | −32.692 | 1.00 | 69.82 | X | O |
| ATOM | 8390 | N | GLY | X | 42 | 40.113 | 32.378 | −31.056 | 1.00 | 60.75 | X | N |
| ATOM | 8391 | CA | GLY | X | 42 | 38.934 | 33.141 | −31.434 | 1.00 | 59.28 | X | C |
| ATOM | 8392 | C | GLY | X | 42 | 37.824 | 32.245 | −31.961 | 1.00 | 64.41 | X | C |
| ATOM | 8393 | O | GLY | X | 42 | 36.695 | 32.698 | −32.170 | 1.00 | 65.31 | X | O |
| ATOM | 8394 | N | LYS | X | 43 | 38.174 | 30.983 | −32.220 | 1.00 | 62.71 | X | N |
| ATOM | 8395 | CA | LYS | X | 43 | 37.256 | 29.947 | −32.702 | 1.00 | 63.08 | X | C |
| ATOM | 8396 | CB | LYS | X | 43 | 37.464 | 29.700 | −34.206 | 1.00 | 63.85 | X | C |
| ATOM | 8397 | CG | LYS | X | 43 | 37.049 | 30.856 | −35.110 | 1.00 | 64.01 | X | C |
| ATOM | 8398 | CD | LYS | X | 43 | 35.541 | 31.126 | −35.034 | 1.00 | 63.27 | X | C |
| ATOM | 8399 | CE | LYS | X | 43 | 35.146 | 32.343 | −35.879 | 1.00 | 63.72 | X | C |
| ATOM | 8400 | NZ | LYS | X | 43 | 35.397 | 33.667 | −35.215 | 1.00 | 63.19 | X | N |
| ATOM | 8401 | C | LYS | X | 43 | 37.652 | 28.697 | −31.911 | 1.00 | 62.13 | X | C |
| ATOM | 8402 | O | LYS | X | 43 | 38.523 | 28.781 | −31.054 | 1.00 | 69.92 | X | O |
| ATOM | 8403 | N | ASN | X | 44 | 37.046 | 27.547 | −32.192 | 1.00 | 57.86 | X | N |
| ATOM | 8404 | CA | ASN | X | 44 | 37.380 | 26.299 | −31.485 | 1.00 | 54.18 | X | C |
| ATOM | 8405 | CB | ASN | X | 44 | 38.785 | 25.813 | −31.857 | 1.00 | 57.92 | X | C |
| ATOM | 8406 | CG | ASN | X | 44 | 39.194 | 24.552 | −31.094 | 1.00 | 64.26 | X | C |
| ATOM | 8407 | OD1 | ASN | X | 44 | 40.101 | 24.582 | −30.249 | 1.00 | 63.63 | X | O |
| ATOM | 8408 | ND2 | ASN | X | 44 | 38.530 | 23.438 | −31.392 | 1.00 | 64.36 | X | N |
| ATOM | 8409 | C | ASN | X | 44 | 37.224 | 26.377 | −29.958 | 1.00 | 51.40 | X | C |
| ATOM | 8410 | O | ASN | X | 44 | 37.995 | 27.040 | −29.257 | 1.00 | 50.97 | X | O |
| ATOM | 8411 | N | LEU | X | 45 | 36.223 | 25.660 | −29.459 | 1.00 | 44.43 | X | N |
| ATOM | 8412 | CA | LEU | X | 45 | 35.902 | 25.625 | −28.045 | 1.00 | 27.42 | X | C |
| ATOM | 8413 | CB | LEU | X | 45 | 34.402 | 25.829 | −27.880 | 1.00 | 25.52 | X | C |
| ATOM | 8414 | CG | LEU | X | 45 | 33.929 | 27.175 | −28.412 | 1.00 | 24.61 | X | C |
| ATOM | 8415 | CD1 | LEU | X | 45 | 32.433 | 27.317 | −28.188 | 1.00 | 27.10 | X | C |
| ATOM | 8416 | CD2 | LEU | X | 45 | 34.668 | 28.290 | −27.698 | 1.00 | 23.37 | X | C |
| ATOM | 8417 | C | LEU | X | 45 | 36.321 | 24.306 | −27.407 | 1.00 | 31.31 | X | C |
| ATOM | 8418 | O | LEU | X | 45 | 36.333 | 23.259 | −28.065 | 1.00 | 25.77 | X | O |
| ATOM | 8419 | N | GLU | X | 46 | 36.643 | 24.362 | −26.115 | 1.00 | 32.47 | X | N |
| ATOM | 8420 | CA | GLU | X | 46 | 37.062 | 23.179 | −25.356 | 1.00 | 36.04 | X | C |
| ATOM | 8421 | CB | GLU | X | 46 | 38.592 | 23.160 | −25.245 | 1.00 | 41.49 | X | C |
| ATOM | 8422 | CG | GLU | X | 46 | 39.308 | 21.881 | −25.703 | 1.00 | 43.15 | X | C |
| ATOM | 8423 | CD | GLU | X | 46 | 40.837 | 22.058 | −25.793 | 1.00 | 44.46 | X | C |
| ATOM | 8424 | OE1 | GLU | X | 46 | 41.313 | 23.214 | −25.888 | 1.00 | 42.63 | X | O |
| ATOM | 8425 | OE2 | GLU | X | 46 | 41.567 | 21.044 | −25.785 | 1.00 | 43.12 | X | O |
| ATOM | 8426 | C | GLU | X | 46 | 36.437 | 23.246 | −23.958 | 1.00 | 32.10 | X | C |
| ATOM | 8427 | O | GLU | X | 46 | 36.445 | 24.301 | −23.321 | 1.00 | 34.74 | X | O |
| ATOM | 8428 | N | TRP | X | 47 | 35.838 | 22.146 | −23.514 | 1.00 | 26.83 | X | N |
| ATOM | 8429 | CA | TRP | X | 47 | 35.228 | 22.095 | −22.187 | 1.00 | 23.69 | X | C |
| ATOM | 8430 | CB | TRP | X | 47 | 34.065 | 21.096 | −22.173 | 1.00 | 25.26 | X | C |
| ATOM | 8431 | CG | TRP | X | 47 | 33.385 | 20.923 | −20.826 | 1.00 | 20.63 | X | C |
| ATOM | 8432 | CD2 | TRP | X | 47 | 33.557 | 19.831 | −19.917 | 1.00 | 16.61 | X | C |
| ATOM | 8433 | CE2 | TRP | X | 47 | 32.664 | 20.035 | −18.844 | 1.00 | 18.53 | X | C |
| ATOM | 8434 | CE3 | TRP | X | 47 | 34.372 | 18.696 | −19.909 | 1.00 | 18.39 | X | C |
| ATOM | 8435 | CD1 | TRP | X | 47 | 32.433 | 21.736 | −20.273 | 1.00 | 19.30 | X | C |
| ATOM | 8436 | NE1 | TRP | X | 47 | 31.994 | 21.208 | −19.083 | 1.00 | 20.66 | X | N |
| ATOM | 8437 | CZ2 | TRP | X | 47 | 32.558 | 19.138 | −17.777 | 1.00 | 15.41 | X | C |
| ATOM | 8438 | CZ3 | TRP | X | 47 | 34.268 | 17.803 | −18.841 | 1.00 | 21.20 | X | C |
| ATOM | 8439 | CH2 | TRP | X | 47 | 33.363 | 18.033 | −17.794 | 1.00 | 12.70 | X | C |
| ATOM | 8440 | C | TRP | X | 47 | 36.293 | 21.702 | −21.153 | 1.00 | 23.95 | X | C |
| ATOM | 8441 | O | TRP | X | 47 | 37.005 | 20.706 | −21.324 | 1.00 | 20.08 | X | O |
| ATOM | 8442 | N | ILE | X | 48 | 36.421 | 22.500 | −20.097 | 1.00 | 23.01 | X | N |
| ATOM | 8443 | CA | ILE | X | 48 | 37.416 | 22.219 | −19.058 | 1.00 | 25.89 | X | C |
| ATOM | 8444 | CB | ILE | X | 48 | 37.951 | 23.523 | −18.431 | 1.00 | 28.44 | X | C |
| ATOM | 8445 | CG2 | ILE | X | 48 | 38.843 | 23.198 | −17.250 | 1.00 | 22.10 | X | C |
| ATOM | 8446 | CG1 | ILE | X | 48 | 38.744 | 24.309 | −19.482 | 1.00 | 25.58 | X | C |
| ATOM | 8447 | CD1 | ILE | X | 48 | 38.781 | 25.775 | −19.229 | 1.00 | 26.05 | X | C |
| ATOM | 8448 | C | ILE | X | 48 | 36.856 | 21.314 | −17.971 | 1.00 | 22.52 | X | C |
| ATOM | 8449 | O | ILE | X | 48 | 37.446 | 20.290 | −17.635 | 1.00 | 27.38 | X | O |
| ATOM | 8450 | N | GLY | X | 49 | 35.694 | 21.695 | −17.453 | 1.00 | 22.27 | X | N |
| ATOM | 8451 | CA | GLY | X | 49 | 35.047 | 20.918 | −16.424 | 1.00 | 13.26 | X | C |
| ATOM | 8452 | C | GLY | X | 49 | 33.762 | 21.588 | −15.961 | 1.00 | 22.65 | X | C |
| ATOM | 8453 | O | GLY | X | 49 | 33.388 | 22.672 | −16.434 | 1.00 | 16.19 | X | O |
| ATOM | 8454 | N | LEU | X | 50 | 33.075 | 20.932 | −15.032 | 1.00 | 18.09 | X | N |
| ATOM | 8455 | CA | LEU | X | 50 | 31.843 | 21.470 | −14.492 | 1.00 | 20.75 | X | C |
| ATOM | 8456 | CB | LEU | X | 50 | 30.643 | 20.595 | −14.922 | 1.00 | 22.05 | X | C |
| ATOM | 8457 | CG | LEU | X | 50 | 30.477 | 19.151 | −14.391 | 1.00 | 24.04 | X | C |
| ATOM | 8458 | CD1 | LEU | X | 50 | 29.869 | 19.143 | −13.001 | 1.00 | 23.31 | X | C |
| ATOM | 8459 | CD2 | LEU | X | 50 | 29.587 | 18.346 | −15.311 | 1.00 | 27.33 | X | C |
| ATOM | 8460 | C | LEU | X | 50 | 31.930 | 21.471 | −12.983 | 1.00 | 23.08 | X | C |
| ATOM | 8461 | O | LEU | X | 50 | 32.727 | 20.723 | −12.381 | 1.00 | 17.57 | X | O |
| ATOM | 8462 | N | VAL | X | 51 | 31.065 | 22.272 | −12.377 | 1.00 | 22.25 | X | N |
| ATOM | 8463 | CA | VAL | X | 51 | 30.968 | 22.321 | −10.928 | 1.00 | 20.80 | X | C |
| ATOM | 8464 | CB | VAL | X | 51 | 31.766 | 23.479 | −10.304 | 1.00 | 20.05 | X | C |
| ATOM | 8465 | CG1 | VAL | X | 51 | 31.193 | 24.837 | −10.760 | 1.00 | 11.08 | X | C |
| ATOM | 8466 | CG2 | VAL | X | 51 | 31.716 | 23.364 | −8.779 | 1.00 | 10.04 | X | C |
| ATOM | 8467 | C | VAL | X | 51 | 29.505 | 22.474 | −10.538 | 1.00 | 24.35 | X | C |
| ATOM | 8468 | O | VAL | X | 51 | 28.754 | 23.216 | −11.178 | 1.00 | 27.23 | X | O |
| ATOM | 8469 | N | HIS | X | 52 | 29.098 | 21.730 | −9.516 | 1.00 | 29.04 | X | N |
| ATOM | 8470 | CA | HIS | X | 52 | 27.730 | 21.794 | −8.993 | 1.00 | 29.66 | X | C |
| ATOM | 8471 | CB | HIS | X | 52 | 27.286 | 20.395 | −8.527 | 1.00 | 25.74 | X | C |
| ATOM | 8472 | CG | HIS | X | 52 | 25.811 | 20.261 | −8.313 | 1.00 | 25.74 | X | C |
| ATOM | 8473 | CD2 | HIS | X | 52 | 24.975 | 19.212 | −8.511 | 1.00 | 23.47 | X | C |
| ATOM | 8474 | ND1 | HIS | X | 52 | 25.029 | 21.287 | −7.828 | 1.00 | 25.93 | X | N |
| ATOM | 8475 | CE1 | HIS | X | 52 | 23.775 | 20.876 | −7.736 | 1.00 | 21.45 | X | C |
| ATOM | 8476 | NE2 | HIS | X | 52 | 23.718 | 19.622 | −8.142 | 1.00 | 21.98 | X | N |
| ATOM | 8477 | C | HIS | X | 52 | 27.906 | 22.741 | −7.806 | 1.00 | 26.71 | X | C |
| ATOM | 8478 | O | HIS | X | 52 | 28.588 | 22.420 | −6.848 | 1.00 | 26.27 | X | O |
| ATOM | 8479 | N | PRO | X | 52A | 27.356 | 23.952 | −7.893 | 1.00 | 31.85 | X | N |
| ATOM | 8480 | CD | PRO | X | 52A | 26.691 | 24.548 | −9.060 | 1.00 | 34.17 | X | C |
| ATOM | 8481 | CA | PRO | X | 52A | 27.482 | 24.939 | −6.814 | 1.00 | 35.96 | X | C |
| ATOM | 8482 | CB | PRO | X | 52A | 26.822 | 26.186 | −7.416 | 1.00 | 38.24 | X | C |
| ATOM | 8483 | CG | PRO | X | 52A | 27.008 | 26.006 | −8.882 | 1.00 | 33.85 | X | C |
| ATOM | 8484 | C | PRO | X | 52A | 26.877 | 24.602 | −5.458 | 1.00 | 37.98 | X | C |
| ATOM | 8485 | O | PRO | X | 52A | 27.193 | 25.265 | −4.476 | 1.00 | 45.27 | X | O |
| ATOM | 8486 | N | HIS | X | 53 | 25.982 | 23.620 | −5.390 | 1.00 | 38.85 | X | N |
| ATOM | 8487 | CA | HIS | X | 53 | 25.380 | 23.282 | −4.102 | 1.00 | 33.08 | X | C |
| ATOM | 8488 | CB | HIS | X | 53 | 24.183 | 22.334 | −4.266 | 1.00 | 29.60 | X | C |
| ATOM | 8489 | CG | HIS | X | 53 | 23.277 | 22.292 | −3.068 | 1.00 | 27.02 | X | C |
| ATOM | 8490 | CD2 | HIS | X | 53 | 22.414 | 23.211 | −2.572 | 1.00 | 21.93 | X | C |
| ATOM | 8491 | ND1 | HIS | X | 53 | 23.196 | 21.201 | −2.230 | 1.00 | 22.88 | X | N |
| ATOM | 8492 | CE1 | HIS | X | 53 | 22.321 | 21.449 | −1.273 | 1.00 | 21.42 | X | C |
| ATOM | 8493 | NE2 | HIS | X | 53 | 21.832 | 22.662 | −1.457 | 1.00 | 19.05 | X | N |
| ATOM | 8494 | C | HIS | X | 53 | 26.386 | 22.659 | −3.136 | 1.00 | 32.14 | X | C |
| ATOM | 8495 | O | HIS | X | 53 | 26.230 | 22.771 | −1.933 | 1.00 | 24.60 | X | O |
| ATOM | 8496 | N | ASN | X | 54 | 27.437 | 22.039 | −3.656 | 1.00 | 29.30 | X | N |
| ATOM | 8497 | CA | ASN | X | 54 | 28.399 | 21.403 | −2.775 | 1.00 | 35.40 | X | C |
| ATOM | 8498 | CB | ASN | X | 54 | 27.869 | 20.027 | −2.353 | 1.00 | 37.45 | X | C |
| ATOM | 8499 | CG | ASN | X | 54 | 28.060 | 18.973 | −3.423 | 1.00 | 33.50 | X | C |
| ATOM | 8500 | OD1 | ASN | X | 54 | 28.331 | 17.814 | −3.117 | 1.00 | 40.37 | X | O |
| ATOM | 8501 | ND2 | ASN | X | 54 | 27.943 | 19.372 | −4.687 | 1.00 | 37.81 | X | N |
| ATOM | 8502 | C | ASN | X | 54 | 29.801 | 21.258 | −3.370 | 1.00 | 37.48 | X | C |
| ATOM | 8503 | O | ASN | X | 54 | 30.621 | 20.465 | −2.866 | 1.00 | 31.36 | X | O |
| ATOM | 8504 | N | GLY | X | 55 | 30.037 | 21.968 | −4.475 | 1.00 | 31.01 | X | N |
| ATOM | 8505 | CA | GLY | X | 55 | 31.327 | 21.948 | −5.140 | 1.00 | 20.77 | X | C |
| ATOM | 8506 | C | GLY | X | 55 | 31.755 | 20.647 | −5.782 | 1.00 | 23.19 | X | C |
| ATOM | 8507 | O | GLY | X | 55 | 32.953 | 20.408 | −5.928 | 1.00 | 24.52 | X | O |
| ATOM | 8508 | N | ALA | X | 56 | 30.805 | 19.775 | −6.117 | 1.00 | 25.44 | X | N |
| ATOM | 8509 | CA | ALA | X | 56 | 31.167 | 18.512 | −6.773 | 1.00 | 28.75 | X | C |
| ATOM | 8510 | CB | ALA | X | 56 | 29.957 | 17.604 | −6.882 | 1.00 | 36.70 | X | C |
| ATOM | 8511 | C | ALA | X | 56 | 31.685 | 18.891 | −8.167 | 1.00 | 29.90 | X | C |
| ATOM | 8512 | O | ALA | X | 56 | 31.106 | 19.755 | −8.837 | 1.00 | 27.56 | X | O |
| ATOM | 8513 | N | ILE | X | 57 | 32.771 | 18.264 | −8.598 | 1.00 | 26.67 | X | N |
| ATOM | 8514 | CA | ILE | X | 57 | 33.361 | 18.603 | −9.885 | 1.00 | 35.18 | X | C |
| ATOM | 8515 | CB | ILE | X | 57 | 34.736 | 19.367 | −9.709 | 1.00 | 34.84 | X | C |
| ATOM | 8516 | CG2 | ILE | X | 57 | 34.524 | 20.849 | −9.501 | 1.00 | 33.34 | X | C |
| ATOM | 8517 | CG1 | ILE | X | 57 | 35.546 | 18.783 | −8.540 | 1.00 | 44.01 | X | C |
| ATOM | 8518 | CD1 | ILE | X | 57 | 36.319 | 17.482 | −8.857 | 1.00 | 49.94 | X | C |
| ATOM | 8519 | C | ILE | X | 57 | 33.620 | 17.420 | −10.802 | 1.00 | 36.58 | X | C |
| ATOM | 8520 | O | ILE | X | 57 | 33.818 | 16.288 | −10.356 | 1.00 | 37.61 | X | O |
| ATOM | 8521 | N | THR | X | 58 | 33.633 | 17.719 | −12.095 | 1.00 | 34.18 | X | N |
| ATOM | 8522 | CA | THR | X | 58 | 33.931 | 16.746 | −13.124 | 1.00 | 33.59 | X | C |
| ATOM | 8523 | CB | THR | X | 58 | 32.671 | 16.283 | −13.901 | 1.00 | 35.86 | X | C |
| ATOM | 8524 | OG1 | THR | X | 58 | 31.696 | 15.776 | −12.982 | 1.00 | 41.47 | X | O |
| ATOM | 8525 | CG2 | THR | X | 58 | 33.036 | 15.168 | −14.885 | 1.00 | 32.35 | X | C |
| ATOM | 8526 | C | THR | X | 58 | 34.858 | 17.519 | −14.048 | 1.00 | 36.37 | X | C |
| ATOM | 8527 | O | THR | X | 58 | 34.502 | 18.599 | −14.536 | 1.00 | 30.24 | X | O |
| ATOM | 8528 | N | TYR | X | 59 | 36.080 | 17.008 | −14.191 | 1.00 | 37.81 | X | N |
| ATOM | 8529 | CA | TYR | X | 59 | 37.113 | 17.612 | −15.029 | 1.00 | 34.02 | X | C |
| ATOM | 8530 | CB | TYR | X | 59 | 38.484 | 17.599 | −14.316 | 1.00 | 34.63 | X | C |
| ATOM | 8531 | CG | TYR | X | 59 | 38.661 | 18.560 | −13.166 | 1.00 | 36.32 | X | C |
| ATOM | 8532 | CD1 | TYR | X | 59 | 38.526 | 19.930 | −13.347 | 1.00 | 34.49 | X | C |
| ATOM | 8533 | CE1 | TYR | X | 59 | 38.681 | 20.818 | −12.279 | 1.00 | 36.39 | X | C |
| ATOM | 8534 | CD2 | TYR | X | 59 | 38.964 | 18.096 | −11.894 | 1.00 | 34.55 | X | C |
| ATOM | 8535 | CE2 | TYR | X | 59 | 39.122 | 18.976 | −10.829 | 1.00 | 37.71 | X | C |
| ATOM | 8536 | CZ | TYR | X | 59 | 38.975 | 20.334 | −11.025 | 1.00 | 34.82 | X | C |
| ATOM | 8537 | OH | TYR | X | 59 | 39.091 | 21.200 | −9.955 | 1.00 | 31.55 | X | O |
| ATOM | 8538 | C | TYR | X | 59 | 37.313 | 16.835 | −16.311 | 1.00 | 29.16 | X | C |
| ATOM | 8539 | O | TYR | X | 59 | 37.210 | 15.613 | −16.331 | 1.00 | 31.05 | X | O |
| ATOM | 8540 | N | ASN | X | 60 | 37.653 | 17.557 | −17.369 | 1.00 | 31.07 | X | N |
| ATOM | 8541 | CA | ASN | X | 60 | 37.989 | 16.943 | −18.644 | 1.00 | 31.18 | X | C |
| ATOM | 8542 | CB | ASN | X | 60 | 38.102 | 18.040 | −19.703 | 1.00 | 31.54 | X | C |
| ATOM | 8543 | CG | ASN | X | 60 | 38.444 | 17.510 | −21.090 | 1.00 | 28.64 | X | C |
| ATOM | 8544 | OD1 | ASN | X | 60 | 38.979 | 16.408 | −21.258 | 1.00 | 29.00 | X | O |
| ATOM | 8545 | ND2 | ASN | X | 60 | 38.132 | 18.310 | −22.098 | 1.00 | 26.01 | X | N |
| ATOM | 8546 | C | ASN | X | 60 | 39.388 | 16.427 | −18.299 | 1.00 | 34.77 | X | C |
| ATOM | 8547 | O | ASN | X | 60 | 40.164 | 17.153 | −17.675 | 1.00 | 35.61 | X | O |
| ATOM | 8548 | N | GLN | X | 61 | 39.712 | 15.189 | −18.654 | 1.00 | 38.21 | X | N |
| ATOM | 8549 | CA | GLN | X | 61 | 41.037 | 14.656 | −18.332 | 1.00 | 42.26 | X | C |
| ATOM | 8550 | CB | GLN | X | 61 | 41.131 | 13.171 | −18.664 | 1.00 | 46.00 | X | C |
| ATOM | 8551 | CG | GLN | X | 61 | 40.074 | 12.315 | −17.960 | 1.00 | 53.32 | X | C |
| ATOM | 8552 | CD | GLN | X | 61 | 40.107 | 12.432 | −16.439 | 1.00 | 55.51 | X | C |
| ATOM | 8553 | OE1 | GLN | X | 61 | 40.803 | 11.668 | −15.762 | 1.00 | 59.46 | X | O |
| ATOM | 8554 | NE2 | GLN | X | 61 | 39.340 | 13.378 | −15.895 | 1.00 | 52.68 | X | N |
| ATOM | 8555 | C | GLN | X | 61 | 42.214 | 15.430 | −18.942 | 1.00 | 43.99 | X | C |
| ATOM | 8556 | O | GLN | X | 61 | 43.313 | 15.410 | −18.389 | 1.00 | 46.51 | X | O |
| ATOM | 8557 | N | LYS | X | 62 | 41.986 | 16.141 | −20.049 | 1.00 | 42.89 | X | N |
| ATOM | 8558 | CA | LYS | X | 62 | 43.049 | 16.934 | −20.677 | 1.00 | 40.56 | X | C |
| ATOM | 8559 | CB | LYS | X | 62 | 42.591 | 17.495 | −22.030 | 1.00 | 44.21 | X | C |
| ATOM | 8560 | CG | LYS | X | 62 | 43.080 | 16.723 | −23.271 | 1.00 | 51.82 | X | C |
| ATOM | 8561 | CD | LYS | X | 62 | 44.520 | 17.098 | −23.666 | 1.00 | 54.51 | X | C |
| ATOM | 8562 | CE | LYS | X | 62 | 44.625 | 18.503 | −24.284 | 1.00 | 53.48 | X | C |
| ATOM | 8563 | NZ | LYS | X | 62 | 44.240 | 18.545 | −25.728 | 1.00 | 48.95 | X | N |
| ATOM | 8564 | C | LYS | X | 62 | 43.494 | 18.096 | −19.776 | 1.00 | 42.12 | X | C |
| ATOM | 8565 | O | LYS | X | 62 | 44.618 | 18.601 | −19.906 | 1.00 | 42.29 | X | O |
| ATOM | 8566 | N | PHE | X | 63 | 42.614 | 18.519 | −18.871 | 1.00 | 32.34 | X | N |
| ATOM | 8567 | CA | PHE | X | 63 | 42.924 | 19.616 | −17.961 | 1.00 | 31.77 | X | C |
| ATOM | 8568 | CB | PHE | X | 63 | 41.907 | 20.744 | −18.131 | 1.00 | 31.07 | X | C |
| ATOM | 8569 | CG | PHE | X | 63 | 41.834 | 21.308 | −19.512 | 1.00 | 36.43 | X | C |
| ATOM | 8570 | CD1 | PHE | X | 63 | 42.597 | 22.420 | −19.866 | 1.00 | 32.03 | X | C |
| ATOM | 8571 | CD2 | PHE | X | 63 | 40.951 | 20.770 | −20.448 | 1.00 | 32.45 | X | C |
| ATOM | 8572 | CE1 | PHE | X | 63 | 42.481 | 22.992 | −21.128 | 1.00 | 38.82 | X | C |
| ATOM | 8573 | CE2 | PHE | X | 63 | 40.828 | 21.334 | −21.712 | 1.00 | 34.61 | X | C |
| ATOM | 8574 | CZ | PHE | X | 63 | 41.593 | 22.449 | −22.058 | 1.00 | 37.27 | X | C |
| ATOM | 8575 | C | PHE | X | 63 | 42.947 | 19.226 | −16.469 | 1.00 | 33.34 | X | C |
| ATOM | 8576 | O | PHE | X | 63 | 43.022 | 20.109 | −15.613 | 1.00 | 25.65 | X | O |
| ATOM | 8577 | N | LYS | X | 64 | 42.912 | 17.929 | −16.150 | 1.00 | 38.80 | X | N |
| ATOM | 8578 | CA | LYS | X | 64 | 42.875 | 17.513 | −14.740 | 1.00 | 45.92 | X | C |
| ATOM | 8579 | CB | LYS | X | 64 | 42.785 | 15.984 | −14.575 | 1.00 | 44.47 | X | C |
| ATOM | 8580 | CG | LYS | X | 64 | 44.089 | 15.250 | −14.788 | 1.00 | 56.46 | X | C |
| ATOM | 8581 | CD | LYS | X | 64 | 43.955 | 13.763 | −14.515 | 1.00 | 60.10 | X | C |
| ATOM | 8582 | CE | LYS | X | 64 | 45.307 | 13.063 | −14.627 | 1.00 | 62.99 | X | C |
| ATOM | 8583 | NZ | LYS | X | 64 | 45.935 | 13.275 | −15.962 | 1.00 | 62.57 | X | N |
| ATOM | 8584 | C | LYS | X | 64 | 43.994 | 18.091 | −13.876 | 1.00 | 46.20 | X | C |
| ATOM | 8585 | O | LYS | X | 64 | 43.789 | 18.363 | −12.693 | 1.00 | 44.57 | X | O |
| ATOM | 8586 | N | ASP | X | 65 | 45.145 | 18.358 | −14.477 | 1.00 | 48.36 | X | N |
| ATOM | 8587 | CA | ASP | X | 65 | 46.234 | 18.905 | −13.699 | 1.00 | 56.75 | X | C |
| ATOM | 8588 | CB | ASP | X | 65 | 47.487 | 18.066 | −13.922 | 1.00 | 62.06 | X | C |
| ATOM | 8589 | CG | ASP | X | 65 | 47.308 | 16.633 | −13.433 | 1.00 | 67.89 | X | C |
| ATOM | 8590 | OD1 | ASP | X | 65 | 46.811 | 16.449 | −12.293 | 1.00 | 67.71 | X | O |
| ATOM | 8591 | OD2 | ASP | X | 65 | 47.636 | 15.691 | −14.192 | 1.00 | 71.73 | X | O |
| ATOM | 8592 | C | ASP | X | 65 | 46.492 | 20.387 | −13.907 | 1.00 | 56.48 | X | C |
| ATOM | 8593 | O | ASP | X | 65 | 47.314 | 20.977 | −13.205 | 1.00 | 61.09 | X | O |
| ATOM | 8594 | N | LYS | X | 66 | 45.747 | 21.007 | −14.819 | 1.00 | 55.01 | X | N |
| ATOM | 8595 | CA | LYS | X | 66 | 45.940 | 22.426 | −15.076 | 1.00 | 51.49 | X | C |
| ATOM | 8596 | CB | LYS | X | 66 | 45.953 | 22.724 | −16.573 | 1.00 | 56.65 | X | C |
| ATOM | 8597 | CG | LYS | X | 66 | 46.890 | 23.872 | −16.921 | 1.00 | 61.44 | X | C |
| ATOM | 8598 | CD | LYS | X | 66 | 46.673 | 24.369 | −18.329 | 1.00 | 66.99 | X | C |
| ATOM | 8599 | CE | LYS | X | 66 | 45.404 | 25.216 | −18.421 | 1.00 | 72.46 | X | C |
| ATOM | 8600 | NZ | LYS | X | 66 | 45.148 | 25.793 | −19.793 | 1.00 | 74.34 | X | N |
| ATOM | 8601 | C | LYS | X | 66 | 44.914 | 23.312 | −14.389 | 1.00 | 50.48 | X | C |
| ATOM | 8602 | O | LYS | X | 66 | 45.264 | 24.346 | −13.810 | 1.00 | 50.89 | X | O |
| ATOM | 8603 | N | ALA | X | 67 | 43.646 | 22.920 | −14.462 | 1.00 | 43.32 | X | N |
| ATOM | 8604 | CA | ALA | X | 67 | 42.588 | 23.710 | −13.844 | 1.00 | 35.88 | X | C |
| ATOM | 8605 | CB | ALA | X | 67 | 41.387 | 23.838 | −14.797 | 1.00 | 28.07 | X | C |
| ATOM | 8606 | C | ALA | X | 67 | 42.137 | 23.160 | −12.496 | 1.00 | 29.93 | X | C |
| ATOM | 8607 | O | ALA | X | 67 | 42.305 | 21.977 | −12.188 | 1.00 | 27.12 | X | O |
| ATOM | 8608 | N | THR | X | 68 | 41.572 | 24.047 | −11.693 | 1.00 | 27.57 | X | N |
| ATOM | 8609 | CA | THR | X | 68 | 41.052 | 23.683 | −10.387 | 1.00 | 33.97 | X | C |
| ATOM | 8610 | CB | THR | X | 68 | 42.061 | 24.032 | −9.262 | 1.00 | 32.88 | X | C |
| ATOM | 8611 | OG1 | THR | X | 68 | 43.229 | 23.220 | −9.423 | 1.00 | 32.17 | X | O |
| ATOM | 8612 | CG2 | THR | X | 68 | 41.456 | 23.781 | −7.880 | 1.00 | 26.50 | X | C |
| ATOM | 8613 | C | THR | X | 68 | 39.767 | 24.487 | −10.254 | 1.00 | 36.99 | X | C |
| ATOM | 8614 | O | THR | X | 68 | 39.800 | 25.723 | −10.224 | 1.00 | 40.56 | X | O |
| ATOM | 8615 | N | LEU | X | 69 | 38.639 | 23.780 | −10.278 | 1.00 | 36.44 | X | N |
| ATOM | 8616 | CA | LEU | X | 69 | 37.327 | 24.409 | −10.188 | 1.00 | 33.93 | X | C |
| ATOM | 8617 | CB | LEU | X | 69 | 36.276 | 23.628 | −10.988 | 1.00 | 35.33 | X | C |
| ATOM | 8618 | CG | LEU | X | 69 | 36.198 | 24.038 | −12.456 | 1.00 | 35.10 | X | C |
| ATOM | 8619 | CD1 | LEU | X | 69 | 37.540 | 23.869 | −13.100 | 1.00 | 29.24 | X | C |
| ATOM | 8620 | CD2 | LEU | X | 69 | 35.180 | 23.200 | −13.184 | 1.00 | 35.39 | X | C |
| ATOM | 8621 | C | LEU | X | 69 | 36.899 | 24.574 | −8.758 | 1.00 | 33.57 | X | C |
| ATOM | 8622 | O | LEU | X | 69 | 36.927 | 23.635 | −7.971 | 1.00 | 37.75 | X | O |
| ATOM | 8623 | N | THR | X | 70 | 36.401 | 25.759 | −8.457 | 1.00 | 30.15 | X | N |
| ATOM | 8624 | CA | THR | X | 70 | 36.013 | 26.071 | −7.109 | 1.00 | 31.39 | X | C |
| ATOM | 8625 | CB | THR | X | 70 | 37.232 | 26.695 | −6.391 | 1.00 | 30.82 | X | C |
| ATOM | 8626 | OG1 | THR | X | 70 | 36.863 | 27.161 | −5.100 | 1.00 | 44.86 | X | O |
| ATOM | 8627 | CG2 | THR | X | 70 | 37.778 | 27.850 | −7.183 | 1.00 | 33.88 | X | C |
| ATOM | 8628 | C | THR | X | 70 | 34.815 | 27.012 | −7.118 | 1.00 | 27.86 | X | C |
| ATOM | 8629 | O | THR | X | 70 | 34.584 | 27.730 | −8.089 | 1.00 | 26.80 | X | O |
| ATOM | 8630 | N | VAL | X | 71 | 34.054 | 27.002 | −6.031 | 1.00 | 24.35 | X | N |
| ATOM | 8631 | CA | VAL | X | 71 | 32.873 | 27.836 | −5.920 | 1.00 | 25.76 | X | C |
| ATOM | 8632 | CB | VAL | X | 71 | 31.598 | 26.999 | −6.316 | 1.00 | 28.94 | X | C |
| ATOM | 8633 | CG1 | VAL | X | 71 | 30.916 | 26.390 | −5.097 | 1.00 | 19.75 | X | C |
| ATOM | 8634 | CG2 | VAL | X | 71 | 30.645 | 27.818 | −7.152 | 1.00 | 26.21 | X | C |
| ATOM | 8635 | C | VAL | X | 71 | 32.789 | 28.402 | −4.493 | 1.00 | 26.39 | X | C |
| ATOM | 8636 | O | VAL | X | 71 | 33.371 | 27.853 | −3.570 | 1.00 | 29.31 | X | O |
| ATOM | 8637 | N | ASP | X | 72 | 32.096 | 29.522 | −4.340 | 1.00 | 29.96 | X | N |
| ATOM | 8638 | CA | ASP | X | 72 | 31.926 | 30.197 | −3.058 | 1.00 | 31.51 | X | C |
| ATOM | 8639 | CB | ASP | X | 72 | 32.892 | 31.389 | −2.980 | 1.00 | 42.19 | X | C |
| ATOM | 8640 | CG | ASP | X | 72 | 32.620 | 32.311 | −1.790 | 1.00 | 50.60 | X | C |
| ATOM | 8641 | OD1 | ASP | X | 72 | 32.136 | 31.844 | −0.735 | 1.00 | 53.09 | X | O |
| ATOM | 8642 | OD2 | ASP | X | 72 | 32.914 | 33.518 | −1.912 | 1.00 | 54.79 | X | O |
| ATOM | 8643 | C | ASP | X | 72 | 30.469 | 30.663 | −2.999 | 1.00 | 31.18 | X | C |
| ATOM | 8644 | O | ASP | X | 72 | 30.073 | 31.612 | −3.677 | 1.00 | 33.11 | X | O |
| ATOM | 8645 | N | ARG | X | 73 | 29.686 | 30.006 | −2.150 | 1.00 | 33.94 | X | N |
| ATOM | 8646 | CA | ARG | X | 73 | 28.257 | 30.281 | −2.026 | 1.00 | 32.03 | X | C |
| ATOM | 8647 | CB | ARG | X | 73 | 27.564 | 29.089 | −1.365 | 1.00 | 32.12 | X | C |
| ATOM | 8648 | CG | ARG | X | 73 | 27.904 | 27.730 | −1.984 | 1.00 | 34.36 | X | C |
| ATOM | 8649 | CD | ARG | X | 73 | 27.452 | 26.565 | −1.100 | 1.00 | 31.23 | X | C |
| ATOM | 8650 | NE | ARG | X | 73 | 28.322 | 25.400 | −1.261 | 1.00 | 37.84 | X | N |
| ATOM | 8651 | CZ | ARG | X | 73 | 29.245 | 25.009 | −0.382 | 1.00 | 41.19 | X | C |
| ATOM | 8652 | NH1 | ARG | X | 73 | 29.437 | 25.694 | 0.739 | 1.00 | 52.13 | X | N |
| ATOM | 8653 | NH2 | ARG | X | 73 | 29.961 | 23.909 | −0.601 | 1.00 | 46.44 | X | N |
| ATOM | 8654 | C | ARG | X | 73 | 27.821 | 31.581 | −1.355 | 1.00 | 36.08 | X | C |
| ATOM | 8655 | O | ARG | X | 73 | 26.738 | 32.097 | −1.656 | 1.00 | 35.48 | X | O |
| ATOM | 8656 | N | SER | X | 74 | 28.641 | 32.123 | −0.460 | 1.00 | 40.15 | X | N |
| ATOM | 8657 | CA | SER | X | 74 | 28.261 | 33.364 | 0.219 | 1.00 | 41.96 | X | C |
| ATOM | 8658 | CB | SER | X | 74 | 29.177 | 33.667 | 1.412 | 1.00 | 46.74 | X | C |
| ATOM | 8659 | OG | SER | X | 74 | 30.548 | 33.670 | 1.052 | 1.00 | 54.76 | X | O |
| ATOM | 8660 | C | SER | X | 74 | 28.184 | 34.544 | −0.737 | 1.00 | 39.11 | X | C |
| ATOM | 8661 | O | SER | X | 74 | 27.360 | 35.433 | −0.553 | 1.00 | 40.18 | X | O |
| ATOM | 8662 | N | SER | X | 75 | 29.007 | 34.524 | −1.785 | 1.00 | 42.04 | X | N |
| ATOM | 8663 | CA | SER | X | 75 | 29.008 | 35.599 | −2.788 | 1.00 | 40.90 | X | C |
| ATOM | 8664 | CB | SER | X | 75 | 30.388 | 36.246 | −2.866 | 1.00 | 37.61 | X | C |
| ATOM | 8665 | OG | SER | X | 75 | 31.371 | 35.262 | −3.102 | 1.00 | 43.59 | X | O |
| ATOM | 8666 | C | SER | X | 75 | 28.563 | 35.125 | −4.188 | 1.00 | 41.64 | X | C |
| ATOM | 8667 | O | SER | X | 75 | 28.683 | 35.866 | −5.169 | 1.00 | 40.16 | X | O |
| ATOM | 8668 | N | THR | X | 76 | 28.047 | 33.894 | −4.257 | 1.00 | 40.04 | X | N |
| ATOM | 8669 | CA | THR | X | 76 | 27.558 | 33.272 | −5.494 | 1.00 | 34.53 | X | C |
| ATOM | 8670 | CB | THR | X | 76 | 26.161 | 33.817 | −5.907 | 1.00 | 32.46 | X | C |
| ATOM | 8671 | OG1 | THR | X | 76 | 26.278 | 35.175 | −6.338 | 1.00 | 32.16 | X | O |
| ATOM | 8672 | CG2 | THR | X | 76 | 25.177 | 33.760 | −4.742 | 1.00 | 28.18 | X | C |
| ATOM | 8673 | C | THR | X | 76 | 28.528 | 33.473 | −6.659 | 1.00 | 37.03 | X | C |
| ATOM | 8674 | O | THR | X | 76 | 28.141 | 33.969 | −7.715 | 1.00 | 36.20 | X | O |
| ATOM | 8675 | N | THR | X | 77 | 29.781 | 33.066 | −6.463 | 1.00 | 32.16 | X | N |
| ATOM | 8676 | CA | THR | X | 77 | 30.826 | 33.231 | −7.469 | 1.00 | 30.19 | X | C |
| ATOM | 8677 | CB | THR | X | 77 | 31.919 | 34.240 | −6.964 | 1.00 | 30.75 | X | C |
| ATOM | 8678 | OG1 | THR | X | 77 | 31.318 | 35.505 | −6.662 | 1.00 | 33.51 | X | O |
| ATOM | 8679 | CG2 | THR | X | 77 | 32.983 | 34.458 | −7.999 | 1.00 | 33.24 | X | C |
| ATOM | 8680 | C | THR | X | 77 | 31.519 | 31.908 | −7.731 | 1.00 | 29.32 | X | C |
| ATOM | 8681 | O | THR | X | 77 | 31.740 | 31.133 | −6.809 | 1.00 | 29.81 | X | O |
| ATOM | 8682 | N | ALA | X | 78 | 31.875 | 31.667 | −8.989 | 1.00 | 25.83 | X | N |
| ATOM | 8683 | CA | ALA | X | 78 | 32.572 | 30.452 | −9.375 | 1.00 | 29.00 | X | C |
| ATOM | 8684 | CB | ALA | X | 78 | 31.884 | 29.822 | −10.577 | 1.00 | 27.66 | X | C |
| ATOM | 8685 | C | ALA | X | 78 | 33.992 | 30.876 | −9.739 | 1.00 | 29.83 | X | C |
| ATOM | 8686 | O | ALA | X | 78 | 34.200 | 32.011 | −10.165 | 1.00 | 29.91 | X | O |
| ATOM | 8687 | N | TYR | X | 79 | 34.961 | 29.981 | −9.577 | 1.00 | 21.74 | X | N |
| ATOM | 8688 | CA | TYR | X | 79 | 36.346 | 30.311 | −9.892 | 1.00 | 26.54 | X | C |
| ATOM | 8689 | CB | TYR | X | 79 | 37.189 | 30.589 | −8.622 | 1.00 | 27.50 | X | C |
| ATOM | 8690 | CG | TYR | X | 79 | 36.731 | 31.685 | −7.683 | 1.00 | 31.66 | X | C |
| ATOM | 8691 | CD1 | TYR | X | 79 | 35.751 | 31.449 | −6.717 | 1.00 | 35.94 | X | C |
| ATOM | 8692 | CE1 | TYR | X | 79 | 35.345 | 32.468 | −5.828 | 1.00 | 40.77 | X | C |
| ATOM | 8693 | CD2 | TYR | X | 79 | 37.297 | 32.952 | −7.736 | 1.00 | 35.57 | X | C |
| ATOM | 8694 | CE2 | TYR | X | 79 | 36.903 | 33.973 | −6.855 | 1.00 | 38.29 | X | C |
| ATOM | 8695 | CZ | TYR | X | 79 | 35.929 | 33.728 | −5.910 | 1.00 | 39.71 | X | C |
| ATOM | 8696 | OH | TYR | X | 79 | 35.525 | 34.744 | −5.073 | 1.00 | 41.61 | X | O |
| ATOM | 8697 | C | TYR | X | 79 | 37.034 | 29.155 | −10.599 | 1.00 | 27.07 | X | C |
| ATOM | 8698 | O | TYR | X | 79 | 36.662 | 27.994 | −10.415 | 1.00 | 23.67 | X | O |
| ATOM | 8699 | N | ILE | X | 80 | 38.023 | 29.493 | −11.424 | 1.00 | 23.85 | X | N |
| ATOM | 8700 | CA | ILE | X | 80 | 38.851 | 28.500 | −12.096 | 1.00 | 25.10 | X | C |
| ATOM | 8701 | CB | ILE | X | 80 | 38.475 | 28.229 | −13.598 | 1.00 | 20.83 | X | C |
| ATOM | 8702 | CG2 | ILE | X | 80 | 38.477 | 29.503 | −14.432 | 1.00 | 23.26 | X | C |
| ATOM | 8703 | CG1 | ILE | X | 80 | 39.449 | 27.220 | −14.197 | 1.00 | 17.28 | X | C |
| ATOM | 8704 | CD1 | ILE | X | 80 | 39.153 | 26.839 | −15.629 | 1.00 | 17.94 | X | C |
| ATOM | 8705 | C | ILE | X | 80 | 40.286 | 29.019 | −11.955 | 1.00 | 31.17 | X | C |
| ATOM | 8706 | O | ILE | X | 80 | 40.582 | 30.187 | −12.244 | 1.00 | 35.64 | X | O |
| ATOM | 8707 | N | GLU | X | 81 | 41.128 | 28.193 | −11.354 | 1.00 | 30.00 | X | N |
| ATOM | 8708 | CA | GLU | X | 81 | 42.513 | 28.546 | −11.168 | 1.00 | 33.88 | X | C |
| ATOM | 8709 | CB | GLU | X | 81 | 42.938 | 28.311 | −9.708 | 1.00 | 34.44 | X | C |
| ATOM | 8710 | CG | GLU | X | 81 | 44.390 | 28.669 | −9.412 | 1.00 | 42.42 | X | C |
| ATOM | 8711 | CD | GLU | X | 81 | 44.731 | 28.644 | −7.928 | 1.00 | 47.52 | X | C |
| ATOM | 8712 | OE1 | GLU | X | 81 | 44.048 | 29.343 | −7.150 | 1.00 | 53.60 | X | O |
| ATOM | 8713 | OE2 | GLU | X | 81 | 45.696 | 27.944 | −7.540 | 1.00 | 52.16 | X | O |
| ATOM | 8714 | C | GLU | X | 81 | 43.354 | 27.717 | −12.119 | 1.00 | 26.65 | X | C |
| ATOM | 8715 | O | GLU | X | 81 | 43.301 | 26.490 | −12.087 | 1.00 | 28.78 | X | O |
| ATOM | 8716 | N | LEU | X | 82 | 44.058 | 28.400 | −13.018 | 1.00 | 28.99 | X | N |
| ATOM | 8717 | CA | LEU | X | 82 | 44.953 | 27.747 | −13.976 | 1.00 | 30.72 | X | C |
| ATOM | 8718 | CB | LEU | X | 82 | 44.857 | 28.403 | −15.358 | 1.00 | 31.43 | X | C |
| ATOM | 8719 | CG | LEU | X | 82 | 43.589 | 28.200 | −16.198 | 1.00 | 32.95 | X | C |
| ATOM | 8720 | CD1 | LEU | X | 82 | 43.038 | 26.803 | −15.982 | 1.00 | 27.35 | X | C |
| ATOM | 8721 | CD2 | LEU | X | 82 | 42.551 | 29.225 | −15.832 | 1.00 | 35.60 | X | C |
| ATOM | 8722 | C | LEU | X | 82 | 46.375 | 27.888 | −13.430 | 1.00 | 34.53 | X | C |
| ATOM | 8723 | O | LEU | X | 82 | 46.780 | 28.986 | −13.022 | 1.00 | 39.23 | X | O |
| ATOM | 8724 | N | VAL | X | 82A | 47.123 | 26.789 | −13.412 | 1.00 | 29.44 | X | N |
| ATOM | 8725 | CA | VAL | X | 82A | 48.487 | 26.811 | −12.893 | 1.00 | 31.27 | X | C |
| ATOM | 8726 | CB | VAL | X | 82A | 48.588 | 25.991 | −11.577 | 1.00 | 25.55 | X | C |
| ATOM | 8727 | CG1 | VAL | X | 82A | 47.548 | 26.468 | −10.601 | 1.00 | 24.38 | X | C |
| ATOM | 8728 | CG2 | VAL | X | 82A | 48.402 | 24.516 | −11.840 | 1.00 | 22.14 | X | C |
| ATOM | 8729 | C | VAL | X | 82A | 49.516 | 26.313 | −13.908 | 1.00 | 35.29 | X | C |
| ATOM | 8730 | O | VAL | X | 82A | 49.146 | 25.741 | −14.937 | 1.00 | 40.32 | X | O |
| ATOM | 8731 | N | ARG | X | 82B | 50.800 | 26.550 | −13.628 | 1.00 | 40.37 | X | N |
| ATOM | 8732 | CA | ARG | X | 82B | 51.889 | 26.129 | −14.519 | 1.00 | 37.40 | X | C |
| ATOM | 8733 | CB | ARG | X | 82B | 52.082 | 24.613 | −14.417 | 1.00 | 40.46 | X | C |
| ATOM | 8734 | CG | ARG | X | 82B | 52.859 | 24.172 | −13.189 | 1.00 | 47.72 | X | C |
| ATOM | 8735 | CD | ARG | X | 82B | 52.681 | 22.690 | −12.877 | 1.00 | 47.85 | X | C |
| ATOM | 8736 | NE | ARG | X | 82B | 51.653 | 22.494 | −11.859 | 1.00 | 56.96 | X | N |
| ATOM | 8737 | CZ | ARG | X | 82B | 50.802 | 21.474 | −11.837 | 1.00 | 60.81 | X | C |
| ATOM | 8738 | NH1 | ARG | X | 82B | 50.856 | 20.543 | −12.785 | 1.00 | 65.85 | X | N |
| ATOM | 8739 | NH2 | ARG | X | 82B | 49.883 | 21.393 | −10.879 | 1.00 | 61.96 | X | N |
| ATOM | 8740 | C | ARG | X | 82B | 51.654 | 26.553 | −15.978 | 1.00 | 38.79 | X | C |
| ATOM | 8741 | O | ARG | X | 82B | 51.925 | 25.802 | −16.924 | 1.00 | 36.70 | X | O |
| ATOM | 8742 | N | LEU | X | 82C | 51.187 | 27.785 | −16.144 | 1.00 | 35.24 | X | N |
| ATOM | 8743 | CA | LEU | X | 82C | 50.882 | 28.331 | −17.456 | 1.00 | 35.22 | X | C |
| ATOM | 8744 | CB | LEU | X | 82C | 50.347 | 29.743 | −17.302 | 1.00 | 29.92 | X | C |
| ATOM | 8745 | CG | LEU | X | 82C | 49.061 | 29.800 | −16.500 | 1.00 | 28.69 | X | C |
| ATOM | 8746 | CD1 | LEU | X | 82C | 48.671 | 31.232 | −16.255 | 1.00 | 27.67 | X | C |
| ATOM | 8747 | CD2 | LEU | X | 82C | 47.974 | 29.070 | −17.254 | 1.00 | 27.83 | X | C |
| ATOM | 8748 | C | LEU | X | 82C | 52.007 | 28.343 | −18.475 | 1.00 | 35.01 | X | C |
| ATOM | 8749 | O | LEU | X | 82C | 53.153 | 28.624 | −18.144 | 1.00 | 45.61 | X | O |
| ATOM | 8750 | N | THR | X | 83 | 51.664 | 27.975 | −19.707 | 1.00 | 39.87 | X | N |
| ATOM | 8751 | CA | THR | X | 83 | 52.580 | 27.992 | −20.844 | 1.00 | 37.76 | X | C |
| ATOM | 8752 | CB | THR | X | 83 | 52.896 | 26.591 | −21.420 | 1.00 | 38.86 | X | C |
| ATOM | 8753 | OG1 | THR | X | 83 | 51.682 | 25.865 | −21.660 | 1.00 | 46.72 | X | O |
| ATOM | 8754 | CG2 | THR | X | 83 | 53.802 | 25.821 | −20.474 | 1.00 | 37.51 | X | C |
| ATOM | 8755 | C | THR | X | 83 | 51.866 | 28.834 | −21.887 | 1.00 | 39.11 | X | C |
| ATOM | 8756 | O | THR | X | 83 | 50.745 | 29.290 | −21.646 | 1.00 | 39.70 | X | O |
| ATOM | 8757 | N | SER | X | 84 | 52.527 | 29.110 | −23.008 | 1.00 | 42.02 | X | N |
| ATOM | 8758 | CA | SER | X | 84 | 51.902 | 29.916 | −24.050 | 1.00 | 38.02 | X | C |
| ATOM | 8759 | CB | SER | X | 84 | 52.895 | 30.191 | −25.172 | 1.00 | 36.48 | X | C |
| ATOM | 8760 | OG | SER | X | 84 | 53.392 | 28.978 | −25.701 | 1.00 | 39.65 | X | O |
| ATOM | 8761 | C | SER | X | 84 | 50.654 | 29.202 | −24.585 | 1.00 | 40.42 | X | C |
| ATOM | 8762 | O | SER | X | 84 | 49.706 | 29.848 | −25.025 | 1.00 | 38.47 | X | O |
| ATOM | 8763 | N | ASN | X | 85 | 50.644 | 27.871 | −24.478 | 1.00 | 42.88 | X | N |
| ATOM | 8764 | CA | ASN | X | 85 | 49.519 | 27.051 | −24.929 | 1.00 | 46.19 | X | C |
| ATOM | 8765 | CB | ASN | X | 85 | 49.859 | 25.556 | −24.807 | 1.00 | 52.46 | X | C |
| ATOM | 8766 | CG | ASN | X | 85 | 51.149 | 25.182 | −25.528 | 1.00 | 58.90 | X | C |
| ATOM | 8767 | OD1 | ASN | X | 85 | 52.154 | 24.831 | −24.898 | 1.00 | 59.33 | X | O |
| ATOM | 8768 | ND2 | ASN | X | 85 | 51.125 | 25.254 | −26.855 | 1.00 | 60.74 | X | N |
| ATOM | 8769 | C | ASN | X | 85 | 48.219 | 27.340 | −24.161 | 1.00 | 42.97 | X | C |
| ATOM | 8770 | O | ASN | X | 85 | 47.150 | 26.914 | −24.591 | 1.00 | 44.03 | X | O |
| ATOM | 8771 | N | ASP | X | 86 | 48.312 | 28.052 | −23.034 | 1.00 | 37.61 | X | N |
| ATOM | 8772 | CA | ASP | X | 86 | 47.141 | 28.369 | −22.225 | 1.00 | 30.34 | X | C |
| ATOM | 8773 | CB | ASP | X | 86 | 47.439 | 28.245 | −20.733 | 1.00 | 30.67 | X | C |
| ATOM | 8774 | CG | ASP | X | 86 | 48.019 | 26.889 | −20.358 | 1.00 | 37.53 | X | C |
| ATOM | 8775 | OD1 | ASP | X | 86 | 47.597 | 25.847 | −20.915 | 1.00 | 37.20 | X | O |
| ATOM | 8776 | OD2 | ASP | X | 86 | 48.914 | 26.863 | −19.490 | 1.00 | 43.63 | X | O |
| ATOM | 8777 | C | ASP | X | 86 | 46.553 | 29.727 | −22.508 | 1.00 | 32.46 | X | C |
| ATOM | 8778 | O | ASP | X | 86 | 45.587 | 30.117 | −21.854 | 1.00 | 37.36 | X | O |
| ATOM | 8779 | N | SER | X | 87 | 47.146 | 30.465 | −23.446 | 1.00 | 31.25 | X | N |
| ATOM | 8780 | CA | SER | X | 87 | 46.622 | 31.784 | −23.819 | 1.00 | 32.44 | X | C |
| ATOM | 8781 | CB | SER | X | 87 | 47.581 | 32.554 | −24.737 | 1.00 | 30.01 | X | C |
| ATOM | 8782 | OG | SER | X | 87 | 48.900 | 32.574 | −24.232 | 1.00 | 30.13 | X | O |
| ATOM | 8783 | C | SER | X | 87 | 45.357 | 31.469 | −24.601 | 1.00 | 31.85 | X | C |
| ATOM | 8784 | O | SER | X | 87 | 45.408 | 30.719 | −25.576 | 1.00 | 34.14 | X | O |
| ATOM | 8785 | N | ALA | X | 88 | 44.233 | 32.015 | −24.144 | 1.00 | 29.48 | X | N |
| ATOM | 8786 | CA | ALA | X | 88 | 42.931 | 31.795 | −24.753 | 1.00 | 28.02 | X | C |
| ATOM | 8787 | CB | ALA | X | 88 | 42.595 | 30.310 | −24.715 | 1.00 | 17.84 | X | C |
| ATOM | 8788 | C | ALA | X | 88 | 41.910 | 32.570 | −23.946 | 1.00 | 25.90 | X | C |
| ATOM | 8789 | O | ALA | X | 88 | 42.244 | 33.138 | −22.913 | 1.00 | 35.07 | X | O |
| ATOM | 8790 | N | VAL | X | 89 | 40.676 | 32.638 | −24.430 | 1.00 | 29.21 | X | N |
| ATOM | 8791 | CA | VAL | X | 89 | 39.622 | 33.321 | −23.681 | 1.00 | 30.37 | X | C |
| ATOM | 8792 | CB | VAL | X | 89 | 38.670 | 34.085 | −24.588 | 1.00 | 27.49 | X | C |
| ATOM | 8793 | CG1 | VAL | X | 89 | 37.561 | 34.698 | −23.756 | 1.00 | 30.33 | X | C |
| ATOM | 8794 | CG2 | VAL | X | 89 | 39.420 | 35.178 | −25.312 | 1.00 | 29.23 | X | C |
| ATOM | 8795 | C | VAL | X | 89 | 38.847 | 32.257 | −22.922 | 1.00 | 30.86 | X | C |
| ATOM | 8796 | O | VAL | X | 89 | 38.497 | 31.229 | −23.490 | 1.00 | 38.91 | X | O |
| ATOM | 8797 | N | TYR | X | 90 | 38.610 | 32.482 | −21.634 | 1.00 | 32.75 | X | N |
| ATOM | 8798 | CA | TYR | X | 90 | 37.898 | 31.504 | −20.816 | 1.00 | 22.56 | X | C |
| ATOM | 8799 | CB | TYR | X | 90 | 38.682 | 31.182 | −19.540 | 1.00 | 21.51 | X | C |
| ATOM | 8800 | CG | TYR | X | 90 | 40.006 | 30.512 | −19.819 | 1.00 | 16.90 | X | C |
| ATOM | 8801 | CD1 | TYR | X | 90 | 41.125 | 31.261 | −20.187 | 1.00 | 18.33 | X | C |
| ATOM | 8802 | CE1 | TYR | X | 90 | 42.349 | 30.643 | −20.482 | 1.00 | 16.64 | X | C |
| ATOM | 8803 | CD2 | TYR | X | 90 | 40.139 | 29.133 | −19.750 | 1.00 | 14.92 | X | C |
| ATOM | 8804 | CE2 | TYR | X | 90 | 41.365 | 28.503 | −20.042 | 1.00 | 15.65 | X | C |
| ATOM | 8805 | CZ | TYR | X | 90 | 42.462 | 29.271 | −20.409 | 1.00 | 17.61 | X | C |
| ATOM | 8806 | OH | TYR | X | 90 | 43.670 | 28.668 | −20.730 | 1.00 | 25.88 | X | O |
| ATOM | 8807 | C | TYR | X | 90 | 36.509 | 31.960 | −20.495 | 1.00 | 25.62 | X | C |
| ATOM | 8808 | O | TYR | X | 90 | 36.295 | 33.124 | −20.151 | 1.00 | 22.63 | X | O |
| ATOM | 8809 | N | TYR | X | 91 | 35.552 | 31.052 | −20.690 | 1.00 | 25.41 | X | N |
| ATOM | 8810 | CA | TYR | X | 91 | 34.152 | 31.352 | −20.432 | 1.00 | 23.46 | X | C |
| ATOM | 8811 | CB | TYR | X | 91 | 33.293 | 31.171 | −21.689 | 1.00 | 24.52 | X | C |
| ATOM | 8812 | CG | TYR | X | 91 | 33.646 | 32.093 | −22.823 | 1.00 | 26.91 | X | C |
| ATOM | 8813 | CD1 | TYR | X | 91 | 34.684 | 31.780 | −23.706 | 1.00 | 25.56 | X | C |
| ATOM | 8814 | CE1 | TYR | X | 91 | 35.024 | 32.634 | −24.732 | 1.00 | 22.46 | X | C |
| ATOM | 8815 | CD2 | TYR | X | 91 | 32.962 | 33.287 | −23.009 | 1.00 | 25.44 | X | C |
| ATOM | 8816 | CE2 | TYR | X | 91 | 33.301 | 34.148 | −24.040 | 1.00 | 26.54 | X | C |
| ATOM | 8817 | CZ | TYR | X | 91 | 34.332 | 33.811 | −24.889 | 1.00 | 23.75 | X | C |
| ATOM | 8818 | OH | TYR | X | 91 | 34.675 | 34.670 | −25.902 | 1.00 | 39.35 | X | O |
| ATOM | 8819 | C | TYR | X | 91 | 33.583 | 30.465 | −19.361 | 1.00 | 22.18 | X | C |
| ATOM | 8820 | O | TYR | X | 91 | 33.951 | 29.304 | −19.236 | 1.00 | 27.72 | X | O |
| ATOM | 8821 | N | CYS | X | 92 | 32.640 | 31.035 | −18.631 | 1.00 | 18.60 | X | N |
| ATOM | 8822 | CA | CYS | X | 92 | 31.913 | 30.374 | −17.562 | 1.00 | 29.72 | X | C |
| ATOM | 8823 | C | CYS | X | 92 | 30.506 | 30.234 | −18.173 | 1.00 | 27.25 | X | C |
| ATOM | 8824 | O | CYS | X | 92 | 29.993 | 31.174 | −18.801 | 1.00 | 22.23 | X | O |
| ATOM | 8825 | CB | CYS | X | 92 | 31.955 | 31.299 | −16.337 | 1.00 | 33.78 | X | C |
| ATOM | 8826 | SG | CYS | X | 92 | 30.698 | 31.200 | −15.026 | 1.00 | 52.01 | X | S |
| ATOM | 8827 | N | ALA | X | 93 | 29.907 | 29.059 | −18.045 | 1.00 | 20.27 | X | N |
| ATOM | 8828 | CA | ALA | X | 93 | 28.594 | 28.843 | −18.653 | 1.00 | 26.15 | X | C |
| ATOM | 8829 | CB | ALA | X | 93 | 28.760 | 28.202 | −20.025 | 1.00 | 21.79 | X | C |
| ATOM | 8830 | C | ALA | X | 93 | 27.617 | 28.041 | −17.818 | 1.00 | 21.52 | X | C |
| ATOM | 8831 | O | ALA | X | 93 | 27.902 | 26.917 | −17.393 | 1.00 | 26.40 | X | O |
| ATOM | 8832 | N | ARG | X | 94 | 26.451 | 28.633 | −17.606 | 1.00 | 20.61 | X | N |
| ATOM | 8833 | CA | ARG | X | 94 | 25.387 | 28.019 | −16.817 | 1.00 | 24.38 | X | C |
| ATOM | 8834 | CB | ARG | X | 94 | 24.381 | 29.090 | −16.427 | 1.00 | 22.94 | X | C |
| ATOM | 8835 | CG | ARG | X | 94 | 23.489 | 28.691 | −15.288 | 1.00 | 23.60 | X | C |
| ATOM | 8836 | CD | ARG | X | 94 | 22.465 | 29.768 | −15.047 | 1.00 | 24.41 | X | C |
| ATOM | 8837 | NE | ARG | X | 94 | 21.304 | 29.664 | −15.928 | 1.00 | 26.59 | X | N |
| ATOM | 8838 | CZ | ARG | X | 94 | 20.079 | 30.053 | −15.577 | 1.00 | 32.44 | X | C |
| ATOM | 8839 | NH1 | ARG | X | 94 | 19.866 | 30.568 | −14.371 | 1.00 | 31.64 | X | N |
| ATOM | 8840 | NH2 | ARG | X | 94 | 19.061 | 29.926 | −16.420 | 1.00 | 33.43 | X | N |
| ATOM | 8841 | C | ARG | X | 94 | 24.679 | 26.873 | −17.569 | 1.00 | 27.22 | X | C |
| ATOM | 8842 | O | ARG | X | 94 | 24.462 | 26.937 | −18.776 | 1.00 | 29.77 | X | O |
| ATOM | 8843 | N | GLU | X | 95 | 24.321 | 25.823 | −16.844 | 1.00 | 32.57 | X | N |
| ATOM | 8844 | CA | GLU | X | 95 | 23.663 | 24.681 | −17.455 | 1.00 | 27.90 | X | C |
| ATOM | 8845 | CB | GLU | X | 95 | 24.664 | 23.557 | −17.686 | 1.00 | 22.09 | X | C |
| ATOM | 8846 | CG | GLU | X | 95 | 24.195 | 22.516 | −18.649 | 1.00 | 23.54 | X | C |
| ATOM | 8847 | CD | GLU | X | 95 | 25.060 | 21.285 | −18.625 | 1.00 | 34.47 | X | C |
| ATOM | 6848 | OE1 | GLU | X | 95 | 26.207 | 21.368 | −18.139 | 1.00 | 36.84 | X | O |
| ATOM | 8849 | OE2 | GLU | X | 95 | 24.590 | 20.220 | −19.086 | 1.00 | 43.91 | X | O |
| ATOM | 8850 | C | GLU | X | 95 | 22.523 | 24.175 | −16.588 | 1.00 | 30.48 | X | C |
| ATOM | 8851 | O | GLU | X | 95 | 22.717 | 23.850 | −15.419 | 1.00 | 26.36 | X | O |
| ATOM | 8852 | N | ASP | X | 96 | 21.329 | 24.145 | −17.173 | 1.00 | 30.03 | X | N |
| ATOM | 8853 | CA | ASP | X | 96 | 20.136 | 23.667 | −16.494 | 1.00 | 21.81 | X | C |
| ATOM | 8854 | CB | ASP | X | 96 | 18.892 | 24.162 | −17.216 | 1.00 | 25.75 | X | C |
| ATOM | 8855 | CG | ASP | X | 96 | 17.635 | 23.462 | −16.752 | 1.00 | 29.86 | X | C |
| ATOM | 8856 | OD1 | ASP | X | 96 | 16.998 | 23.957 | −15.805 | 1.00 | 36.52 | X | O |
| ATOM | 8857 | OD2 | ASP | X | 96 | 17.297 | 22.401 | −17.319 | 1.00 | 31.16 | X | O |
| ATOM | 8858 | C | ASP | X | 96 | 20.110 | 22.144 | −16.426 | 1.00 | 21.02 | X | C |
| ATOM | 8859 | O | ASP | X | 96 | 20.649 | 21.447 | −17.289 | 1.00 | 15.08 | X | O |
| ATOM | 8860 | N | PHE | X | 97 | 19.507 | 21.615 | −15.378 | 1.00 | 17.10 | X | N |
| ATOM | 8861 | CA | PHE | X | 97 | 19.431 | 20.182 | −15.296 | 1.00 | 18.79 | X | C |
| ATOM | 8862 | CB | PHE | X | 97 | 20.533 | 19.586 | −14.413 | 1.00 | 16.37 | X | C |
| ATOM | 8863 | CG | PHE | X | 97 | 20.656 | 20.205 | −13.050 | 1.00 | 19.88 | X | C |
| ATOM | 8864 | CD1 | PHE | X | 97 | 19.552 | 20.686 | −12.368 | 1.00 | 25.95 | X | C |
| ATOM | 8865 | CD2 | PHE | X | 97 | 21.890 | 20.235 | −12.415 | 1.00 | 23.88 | X | C |
| ATOM | 8866 | CE1 | PHE | X | 97 | 19.670 | 21.180 | −11.074 | 1.00 | 29.40 | X | C |
| ATOM | 8867 | CE2 | PHE | X | 97 | 22.019 | 20.726 | −11.119 | 1.00 | 29.77 | X | C |
| ATOM | 8868 | CZ | PHE | X | 97 | 20.908 | 21.198 | −10.447 | 1.00 | 29.26 | X | C |
| ATOM | 8869 | C | PHE | X | 97 | 18.059 | 19.723 | −14.904 | 1.00 | 14.09 | X | C |
| ATOM | 8870 | O | PHE | X | 97 | 17.890 | 18.582 | −14.492 | 1.00 | 18.59 | X | O |
| ATOM | 8871 | N | ARG | X | 98 | 17.075 | 20.594 | −15.122 | 1.00 | 16.79 | X | N |
| ATOM | 8872 | CA | ARG | X | 98 | 15.674 | 20.293 | −14.805 | 1.00 | 25.00 | X | C |
| ATOM | 8873 | CB | ARG | X | 98 | 15.024 | 21.448 | −14.034 | 1.00 | 23.68 | X | C |
| ATOM | 8874 | CG | ARG | X | 98 | 15.440 | 21.502 | −12.571 | 1.00 | 27.40 | X | C |
| ATOM | 8875 | CD | ARG | X | 98 | 14.553 | 22.452 | −11.769 | 1.00 | 22.63 | X | C |
| ATOM | 8876 | NE | ARG | X | 98 | 15.084 | 22.640 | −10.433 | 1.00 | 21.71 | X | N |
| ATOM | 8877 | CZ | ARG | X | 98 | 16.140 | 23.402 | −10.154 | 1.00 | 28.10 | X | C |
| ATOM | 8878 | NH1 | ARG | X | 98 | 16.770 | 24.049 | −11.126 | 1.00 | 21.53 | X | N |
| ATOM | 8879 | NH2 | ARG | X | 98 | 16.562 | 23.531 | −8.900 | 1.00 | 26.84 | X | N |
| ATOM | 8880 | C | ARG | X | 98 | 14.766 | 19.884 | −15.970 | 1.00 | 26.43 | X | C |
| ATOM | 8881 | O | ARG | X | 98 | 13.965 | 18.956 | −15.814 | 1.00 | 24.82 | X | O |
| ATOM | 8882 | N | TYR | X | 99 | 14.880 | 20.563 | −17.118 | 1.00 | 28.49 | X | N |
| ATOM | 8883 | CA | TYR | X | 99 | 14.033 | 20.245 | −18.266 | 1.00 | 29.95 | X | C |
| ATOM | 8884 | CB | TYR | X | 99 | 12.612 | 20.790 | −18.039 | 1.00 | 28.09 | X | C |
| ATOM | 8885 | CG | TYR | X | 99 | 12.485 | 22.290 | −17.889 | 1.00 | 26.14 | X | C |
| ATOM | 8886 | CD1 | TYR | X | 99 | 12.542 | 22.889 | −16.631 | 1.00 | 29.85 | X | C |
| ATOM | 8887 | CE1 | TYR | X | 99 | 12.313 | 24.258 | −16.472 | 1.00 | 28.77 | X | C |
| ATOM | 8888 | CD2 | TYR | X | 99 | 12.208 | 23.102 | −18.995 | 1.00 | 22.45 | X | C |
| ATOM | 8889 | CE2 | TYR | X | 99 | 11.978 | 24.468 | −18.852 | 1.00 | 20.86 | X | C |
| ATOM | 8890 | CZ | TYR | X | 99 | 12.027 | 25.037 | −17.585 | 1.00 | 28.39 | X | C |
| ATOM | 8891 | OH | TYR | X | 99 | 11.741 | 26.366 | −17.416 | 1.00 | 28.21 | X | O |
| ATOM | 8892 | C | TYR | X | 99 | 14.527 | 20.606 | −19.682 | 1.00 | 30.56 | X | C |
| ATOM | 8893 | O | TYR | X | 99 | 14.077 | 20.014 | −20.666 | 1.00 | 32.15 | X | O |
| ATOM | 8894 | N | HIS | X | 100 | 15.365 | 21.627 | −19.791 | 1.00 | 29.41 | X | N |
| ATOM | 8895 | CA | HIS | X | 100 | 15.944 | 22.055 | −21.064 | 1.00 | 31.52 | X | C |
| ATOM | 8896 | CB | HIS | X | 100 | 15.492 | 23.476 | −21.402 | 1.00 | 29.31 | X | C |
| ATOM | 8897 | CG | HIS | X | 100 | 16.010 | 24.519 | −20.459 | 1.00 | 30.70 | X | C |
| ATOM | 8898 | CD2 | HIS | X | 100 | 15.619 | 24.870 | −19.211 | 1.00 | 31.10 | X | C |
| ATOM | 8899 | ND1 | HIS | X | 100 | 17.115 | 25.292 | −20.743 | 1.00 | 30.07 | X | N |
| ATOM | 8900 | CE1 | HIS | X | 100 | 17.383 | 26.073 | −19.713 | 1.00 | 29.34 | X | C |
| ATOM | 8901 | NE2 | HIS | X | 100 | 16.489 | 25.837 | −18.771 | 1.00 | 33.17 | X | N |
| ATOM | 8902 | C | HIS | X | 100 | 17.411 | 22.019 | −20.672 | 1.00 | 38.22 | X | C |
| ATOM | 8903 | O | HIS | X | 100 | 17.940 | 22.938 | −20.050 | 1.00 | 52.59 | X | O |
| ATOM | 8904 | N | TYR | X | 100A | 18.084 | 20.932 | −20.982 | 1.00 | 36.89 | X | N |
| ATOM | 8905 | CA | TYR | X | 100A | 19.468 | 20.796 | −20.530 | 1.00 | 25.45 | X | C |
| ATOM | 8906 | CB | TYR | X | 100A | 19.796 | 19.320 | −20.475 | 1.00 | 21.57 | X | C |
| ATOM | 8907 | CG | TYR | X | 100A | 18.724 | 18.590 | −19.689 | 1.00 | 24.77 | X | C |
| ATOM | 8908 | CD1 | TYR | X | 100A | 17.411 | 18.463 | −20.191 | 1.00 | 21.18 | X | C |
| ATOM | 8909 | CE1 | TYR | X | 100A | 16.410 | 17.866 | −19.438 | 1.00 | 22.62 | X | C |
| ATOM | 8910 | CD2 | TYR | X | 100A | 18.989 | 18.087 | −18.418 | 1.00 | 18.04 | X | C |
| ATOM | 8911 | CE2 | TYR | X | 100A | 17.994 | 17.484 | −17.673 | 1.00 | 26.00 | X | C |
| ATOM | 8912 | CZ | TYR | X | 100A | 16.711 | 17.381 | −18.189 | 1.00 | 21.62 | X | C |
| ATOM | 8913 | OH | TYR | X | 100A | 15.747 | 16.800 | −17.433 | 1.00 | 29.26 | X | O |
| ATOM | 8914 | C | TYR | X | 100A | 20.478 | 21.622 | −21.304 | 1.00 | 24.96 | X | C |
| ATOM | 8915 | O | TYR | X | 100A | 21.570 | 21.171 | −21.639 | 1.00 | 29.62 | X | O |
| ATOM | 8916 | N | SER | X | 100B | 20.123 | 22.887 | −21.450 | 1.00 | 19.69 | X | N |
| ATOM | 8917 | CA | SER | X | 100B | 20.887 | 23.871 | −22.185 | 1.00 | 28.47 | X | C |
| ATOM | 8918 | CB | SER | X | 100B | 19.889 | 24.843 | −22.813 | 1.00 | 24.33 | X | C |
| ATOM | 8919 | OG | SER | X | 100B | 20.534 | 26.015 | −23.261 | 1.00 | 32.37 | X | O |
| ATOM | 8920 | C | SER | X | 100B | 21.925 | 24.696 | −21.430 | 1.00 | 27.32 | X | C |
| ATOM | 8921 | O | SER | X | 100B | 21.769 | 25.001 | −20.253 | 1.00 | 24.82 | X | O |
| ATOM | 8922 | N | MET | X | 100C | 22.977 | 25.072 | −22.148 | 1.00 | 26.21 | X | N |
| ATOM | 8923 | CA | MET | X | 100C | 24.005 | 25.950 | −21.620 | 1.00 | 16.26 | X | C |
| ATOM | 8924 | CB | MET | X | 100C | 25.337 | 25.641 | −22.276 | 1.00 | 14.61 | X | C |
| ATOM | 8925 | CG | MET | X | 100C | 25.872 | 24.296 | −21.819 | 1.00 | 16.36 | X | C |
| ATOM | 8926 | SD | MET | X | 100C | 27.629 | 24.104 | −22.067 | 1.00 | 28.90 | X | S |
| ATOM | 8927 | CE | MET | X | 100C | 27.918 | 22.499 | −21.371 | 1.00 | 31.49 | X | C |
| ATOM | 8928 | C | MET | X | 100C | 23.453 | 27.311 | −22.043 | 1.00 | 21.51 | X | C |
| ATOM | 8929 | O | MET | X | 100C | 23.812 | 27.847 | −23.096 | 1.00 | 19.22 | X | O |
| ATOM | 8930 | N | ASP | X | 101 | 22.527 | 27.830 | −21.237 | 1.00 | 16.68 | X | N |
| ATOM | 8931 | CA | ASP | X | 101 | 21.840 | 29.075 | −21.560 | 1.00 | 19.90 | X | C |
| ATOM | 8932 | CB | ASP | X | 101 | 20.452 | 29.133 | −20.892 | 1.00 | 21.09 | X | C |
| ATOM | 8933 | CG | ASP | X | 101 | 20.471 | 28.804 | −19.388 | 1.00 | 29.28 | X | C |
| ATOM | 8934 | OD1 | ASP | X | 101 | 21.544 | 28.470 | −18.813 | 1.00 | 22.50 | X | O |
| ATOM | 8935 | OD2 | ASP | X | 101 | 19.365 | 28.875 | −18.788 | 1.00 | 23.69 | X | O |
| ATOM | 8936 | C | ASP | X | 101 | 22.561 | 30.395 | −21.421 | 100 | 26.10 | X | C |
| ATOM | 8937 | O | ASP | X | 101 | 22.695 | 31.130 | −22.401 | 1.00 | 28.27 | X | O |
| ATOM | 8938 | N | TYR | X | 102 | 22.977 | 30.732 | −20.204 | 1.00 | 30.25 | X | N |
| ATOM | 8939 | CA | TYR | X | 102 | 23.689 | 31.984 | −19.984 | 1.00 | 28.55 | X | C |
| ATOM | 8940 | CB | TYR | X | 102 | 23.147 | 32.706 | −18.763 | 1.00 | 29.49 | X | C |
| ATOM | 8941 | CG | TYR | X | 102 | 21.782 | 33.308 | −18.994 | 1.00 | 33.27 | X | C |
| ATOM | 8942 | CD1 | TYR | X | 102 | 20.622 | 32.571 | −18.758 | 1.00 | 32.54 | X | C |
| ATOM | 8943 | CE1 | TYR | X | 102 | 19.356 | 33.129 | −18.973 | 1.00 | 32.01 | X | C |
| ATOM | 8944 | CD2 | TYR | X | 102 | 21.650 | 34.618 | −19.451 | 1.00 | 33.70 | X | C |
| ATOM | 8945 | CE2 | TYR | X | 102 | 20.395 | 35.183 | −19.666 | 1.00 | 34.88 | X | C |
| ATOM | 8946 | CZ | TYR | X | 102 | 19.253 | 34.433 | −19.423 | 1.00 | 34.58 | X | C |
| ATOM | 8947 | OH | TYR | X | 102 | 18.013 | 35.004 | −19.612 | 1.00 | 42.26 | X | O |
| ATOM | 8948 | C | TYR | X | 102 | 25.194 | 31.810 | −19.895 | 1.00 | 30.32 | X | C |
| ATOM | 8949 | O | TYR | X | 102 | 25.697 | 30.941 | −19.174 | 1.00 | 34.94 | X | O |
| ATOM | 8950 | N | TRP | X | 103 | 25.902 | 32.621 | −20.680 | 1.00 | 28.65 | X | N |
| ATOM | 8951 | CA | TRP | X | 103 | 27.355 | 32.591 | −20.732 | 1.00 | 25.06 | X | C |
| ATOM | 8952 | CB | TRP | X | 103 | 27.827 | 32.322 | −22.172 | 1.00 | 22.90 | X | C |
| ATOM | 8953 | CG | TRP | X | 103 | 27.547 | 30.923 | −22.670 | 1.00 | 20.16 | X | C |
| ATOM | 8954 | CD2 | TRP | X | 103 | 28.509 | 29.956 | −23.130 | 1.00 | 15.29 | X | C |
| ATOM | 8955 | CE2 | TRP | X | 103 | 27.800 | 28.775 | −23.435 | 1.00 | 13.38 | X | C |
| ATOM | 8956 | CE3 | TRP | X | 103 | 29.893 | 29.973 | −23.309 | 1.00 | 17.16 | X | C |
| ATOM | 8957 | CD1 | TRP | X | 103 | 26.333 | 30.306 | −22.727 | 1.00 | 15.93 | X | C |
| ATOM | 8958 | NE1 | TRP | X | 103 | 26.481 | 29.015 | −23.174 | 1.00 | 19.15 | X | N |
| ATOM | 8959 | CZ2 | TRP | X | 103 | 28.429 | 27.613 | −23.912 | 1.00 | 18.60 | X | C |
| ATOM | 8960 | CZ3 | TRP | X | 103 | 30.529 | 28.808 | −23.786 | 1.00 | 15.55 | X | C |
| ATOM | 8961 | CH2 | TRP | X | 103 | 29.787 | 27.648 | −24.082 | 1.00 | 14.99 | X | C |
| ATOM | 8962 | C | TRP | X | 103 | 27.923 | 33.916 | −20.236 | 1.00 | 27.51 | X | C |
| ATOM | 8963 | O | TRP | X | 103 | 27.275 | 34.965 | −20.359 | 1.00 | 33.45 | X | O |
| ATOM | 8964 | N | GLY | X | 104 | 29.088 | 33.848 | −19.587 | 1.00 | 29.36 | X | N |
| ATOM | 8965 | CA | GLY | X | 104 | 29.741 | 35.051 | −19.112 | 1.00 | 26.16 | X | C |
| ATOM | 8966 | C | GLY | X | 104 | 30.402 | 35.689 | −20.324 | 1.00 | 35.38 | X | C |
| ATOM | 8967 | O | GLY | X | 104 | 30.574 | 35.011 | −21.352 | 1.00 | 34.50 | X | O |
| ATOM | 8968 | N | GLN | X | 105 | 30.778 | 36.968 | −20.239 | 1.00 | 29.96 | X | N |
| ATOM | 8969 | CA | GLN | X | 105 | 31.410 | 37.613 | −21.389 | 1.00 | 32.42 | X | C |
| ATOM | 8970 | CB | GLN | X | 105 | 31.427 | 39.151 | −21.265 | 1.00 | 36.57 | X | C |
| ATOM | 8971 | CG | GLN | X | 105 | 32.410 | 39.722 | −20.258 | 1.00 | 46.80 | X | C |
| ATOM | 8972 | CD | GLN | X | 105 | 31.991 | 39.441 | −18.838 | 1.00 | 46.47 | X | C |
| ATOM | 8973 | OE1 | GLN | X | 105 | 32.354 | 38.422 | −18.261 | 1.00 | 43.28 | X | O |
| ATOM | 8974 | NE2 | GLN | X | 105 | 31.181 | 40.330 | −18.281 | 1.00 | 55.25 | X | N |
| ATOM | 8975 | C | GLN | X | 105 | 32.809 | 37.071 | −21.710 | 1.00 | 29.13 | X | C |
| ATOM | 8976 | O | GLN | X | 105 | 33.353 | 37.384 | −22.772 | 1.00 | 29.99 | X | O |
| ATOM | 8977 | N | GLY | X | 106 | 33.353 | 36.226 | −20.824 | 1.00 | 27.47 | X | N |
| ATOM | 8978 | CA | GLY | X | 106 | 34.678 | 35.638 | −21.022 | 1.00 | 31.02 | X | C |
| ATOM | 8979 | C | GLY | X | 106 | 35.856 | 36.417 | −20.445 | 1.00 | 27.96 | X | C |
| ATOM | 8980 | O | GLY | X | 106 | 35.760 | 37.622 | −20.211 | 1.00 | 29.92 | X | O |
| ATOM | 8981 | N | THR | X | 107 | 36.960 | 35.723 | −20.195 | 1.00 | 30.07 | X | N |
| ATOM | 8982 | CA | THR | X | 107 | 38.167 | 36.343 | −19.649 | 1.00 | 32.13 | X | C |
| ATOM | 8983 | CB | THR | X | 107 | 38.416 | 35.940 | −18.174 | 1.00 | 35.24 | X | C |
| ATOM | 8984 | OG1 | THR | X | 107 | 37.304 | 36.355 | −17.371 | 1.00 | 36.52 | X | O |
| ATOM | 8985 | CG2 | THR | X | 107 | 39.695 | 36.605 | −17.636 | 1.00 | 33.93 | X | C |
| ATOM | 8986 | C | THR | X | 107 | 39.380 | 35.961 | −20.488 | 1.00 | 32.53 | X | C |
| ATOM | 8987 | O | THR | X | 107 | 39.719 | 34.778 | −20.628 | 1.00 | 36.99 | X | O |
| ATOM | 8988 | N | SER | X | 108 | 40.032 | 36.977 | −21.045 | 1.00 | 33.32 | X | N |
| ATOM | 8989 | CA | SER | X | 108 | 41.199 | 36.762 | −21.893 | 1.00 | 33.64 | X | C |
| ATOM | 8990 | CB | SER | X | 108 | 41.382 | 37.948 | −22.830 | 1.00 | 34.14 | X | C |
| ATOM | 8991 | OG | SER | X | 108 | 42.327 | 37.641 | −23.833 | 1.00 | 38.80 | X | O |
| ATOM | 8992 | C | SER | X | 108 | 42.481 | 36.526 | −21.114 | 1.00 | 33.21 | X | C |
| ATOM | 8993 | O | SER | X | 108 | 42.862 | 37.336 | −20.276 | 1.00 | 35.67 | X | O |
| ATOM | 8994 | N | VAL | X | 109 | 43.133 | 35.404 | −21.380 | 1.00 | 33.16 | X | N |
| ATOM | 8995 | CA | VAL | X | 109 | 44.379 | 35.081 | −20.714 | 1.00 | 33.39 | X | C |
| ATOM | 8996 | CB | VAL | X | 109 | 44.324 | 33.717 | −19.991 | 1.00 | 36.74 | X | C |
| ATOM | 8997 | CG1 | VAL | X | 109 | 45.704 | 33.362 | −19.420 | 1.00 | 34.12 | X | C |
| ATOM | 8998 | CG2 | VAL | X | 109 | 43.289 | 33.748 | −18.887 | 1.00 | 41.13 | X | C |
| ATOM | 8999 | C | VAL | X | 109 | 45.493 | 35.034 | −21.742 | 1.00 | 37.58 | X | C |
| ATOM | 9000 | O | VAL | X | 109 | 45.408 | 34.323 | −22.744 | 1.00 | 41.51 | X | O |
| ATOM | 9001 | N | THR | X | 110 | 46.531 | 35.820 | −21.499 | 1.00 | 39.75 | X | N |
| ATOM | 9002 | CA | THR | X | 110 | 47.679 | 35.846 | −22.380 | 1.00 | 34.06 | X | C |
| ATOM | 9003 | CB | THR | X | 110 | 47.909 | 37.238 | −22.968 | 1.00 | 36.54 | X | C |
| ATOM | 9004 | OG1 | THR | X | 110 | 46.715 | 37.685 | −23.629 | 1.00 | 36.57 | X | O |
| ATOM | 9005 | CG2 | THR | X | 110 | 49.055 | 37.192 | −23.959 | 1.00 | 34.26 | X | C |
| ATOM | 9006 | C | THR | X | 110 | 48.891 | 35.440 | −21.559 | 1.00 | 36.67 | X | C |
| ATOM | 9007 | O | THR | X | 110 | 49.177 | 36.055 | −20.523 | 1.00 | 25.67 | X | O |
| ATOM | 9008 | N | VAL | X | 111 | 49.538 | 34.360 | −22.006 | 1.00 | 33.82 | X | N |
| ATOM | 9009 | CA | VAL | X | 111 | 50.724 | 33.797 | −21.387 | 1.00 | 36.82 | X | C |
| ATOM | 9010 | CB | VAL | X | 111 | 50.514 | 32.322 | −21.016 | 1.00 | 39.69 | X | C |
| ATOM | 9011 | CG1 | VAL | X | 111 | 51.674 | 31.837 | −20.174 | 1.00 | 39.46 | X | C |
| ATOM | 9012 | CG2 | VAL | X | 111 | 49.202 | 32.137 | −20.281 | 1.00 | 41.55 | X | C |
| ATOM | 9013 | C | VAL | X | 111 | 51.878 | 33.872 | −22.392 | 1.00 | 42.90 | X | C |
| ATOM | 9014 | O | VAL | X | 111 | 51.894 | 33.156 | −23.404 | 1.00 | 40.94 | X | O |
| ATOM | 9015 | N | SER | X | 112 | 52.855 | 34.725 | −22.086 | 1.00 | 46.54 | X | N |
| ATOM | 9016 | CA | SER | X | 112 | 54.026 | 34.925 | −22.945 | 1.00 | 45.63 | X | C |
| ATOM | 9017 | CB | SER | X | 112 | 53.746 | 36.025 | −23.973 | 1.00 | 40.84 | X | C |
| ATOM | 9018 | OG | SER | X | 112 | 54.869 | 36.202 | −24.814 | 1.00 | 42.66 | X | O |
| ATOM | 9019 | C | SER | X | 112 | 55.268 | 35.321 | −22.151 | 1.00 | 48.35 | X | C |
| ATOM | 9020 | O | SER | X | 112 | 55.165 | 35.963 | −21.097 | 1.00 | 48.55 | X | O |
| ATOM | 9021 | N | SER | X | 113 | 56.439 | 34.942 | −22.670 | 1.00 | 46.60 | X | N |
| ATOM | 9022 | CA | SER | X | 113 | 57.715 | 35.284 | −22.037 | 1.00 | 44.10 | X | C |
| ATOM | 9023 | CB | SER | X | 113 | 58.697 | 34.102 | −22.121 | 1.00 | 43.05 | X | C |
| ATOM | 9024 | OG | SER | X | 113 | 59.119 | 33.830 | −23.448 | 1.00 | 48.04 | X | O |
| ATOM | 9025 | C | SER | X | 113 | 58.303 | 36.519 | −22.731 | 1.00 | 42.97 | X | C |
| ATOM | 9026 | O | SER | X | 113 | 59.470 | 36.853 | −22.562 | 1.00 | 45.07 | X | O |
| ATOM | 9027 | N | ALA | X | 114 | 57.475 | 37.181 | −23.524 | 1.00 | 40.99 | X | N |
| ATOM | 9028 | CA | ALA | X | 114 | 57.883 | 38.355 | −24.268 | 1.00 | 40.30 | X | C |
| ATOM | 9029 | CB | ALA | X | 114 | 56.898 | 38.617 | −25.401 | 1.00 | 42.37 | X | C |
| ATOM | 9030 | C | ALA | X | 114 | 57.981 | 39.580 | −23.387 | 1.00 | 40.96 | X | C |
| ATOM | 9031 | O | ALA | X | 114 | 57.211 | 39.736 | −22.432 | 1.00 | 45.35 | X | O |
| ATOM | 9032 | N | LYS | X | 115 | 58.920 | 40.458 | −23.726 | 1.00 | 36.81 | X | N |
| ATOM | 9033 | CA | LYS | X | 115 | 59.108 | 41.700 | −22.982 | 1.00 | 37.78 | X | C |
| ATOM | 9034 | CB | LYS | X | 115 | 60.603 | 41.977 | −22.744 | 1.00 | 36.99 | X | C |
| ATOM | 9035 | CG | LYS | X | 115 | 61.355 | 42.440 | −23.984 | 1.00 | 40.59 | X | C |
| ATOM | 9036 | CD | LYS | X | 115 | 62.799 | 41.969 | −24.004 | 1.00 | 46.00 | X | C |
| ATOM | 9037 | CE | LYS | X | 115 | 63.480 | 42.368 | −25.314 | 1.00 | 50.37 | X | C |
| ATOM | 9038 | NZ | LYS | X | 115 | 64.841 | 41.751 | −25.466 | 1.00 | 52.10 | X | N |
| ATOM | 9039 | C | LYS | X | 115 | 58.497 | 42.832 | −23.811 | 1.00 | 36.67 | X | C |
| ATOM | 9040 | O | LYS | X | 115 | 58.292 | 42.685 | −25.022 | 1.00 | 30.22 | X | O |
| ATOM | 9041 | N | THR | X | 116 | 58.204 | 43.959 | −23.169 | 1.00 | 28.96 | X | N |
| ATOM | 9042 | CA | THR | X | 116 | 57.643 | 45.081 | −23.903 | 1.00 | 34.51 | X | C |
| ATOM | 9043 | CB | THR | X | 116 | 57.350 | 46.255 | −22.993 | 1.00 | 29.41 | X | C |
| ATOM | 9044 | OG1 | THR | X | 116 | 56.449 | 45.834 | −21.963 | 1.00 | 36.39 | X | O |
| ATOM | 9045 | CG2 | THR | X | 116 | 56.717 | 47.389 | −23.796 | 1.00 | 35.27 | X | C |
| ATOM | 9046 | C | THR | X | 116 | 58.605 | 45.534 | −24.997 | 1.00 | 35.16 | X | C |
| ATOM | 9047 | O | THR | X | 116 | 59.736 | 45.921 | −24.711 | 1.00 | 40.08 | X | O |
| ATOM | 9048 | N | THR | X | 117 | 58.182 | 45.407 | −26.251 | 1.00 | 29.59 | X | N |
| ATOM | 9049 | CA | THR | X | 117 | 59.015 | 45.828 | −27.370 | 1.00 | 26.52 | X | C |
| ATOM | 9050 | CB | THR | X | 117 | 59.827 | 44.656 | −28.035 | 1.00 | 26.38 | X | C |
| ATOM | 9051 | OG1 | THR | X | 117 | 59.664 | 44.681 | −29.454 | 1.00 | 34.21 | X | O |
| ATOM | 9052 | CG2 | THR | X | 117 | 59.434 | 43.312 | −27.511 | 1.00 | 27.43 | X | C |
| ATOM | 9053 | C | THR | X | 117 | 58.198 | 46.610 | −28.387 | 1.00 | 26.48 | X | C |
| ATOM | 9054 | O | THR | X | 117 | 57.097 | 46.213 | −28.764 | 1.00 | 30.64 | X | O |
| ATOM | 9055 | N | PRO | X | 118 | 58.726 | 47.761 | −28.824 | 1.00 | 25.49 | X | N |
| ATOM | 9056 | CD | PRO | X | 118 | 60.065 | 48.295 | −28.492 | 1.00 | 20.09 | X | C |
| ATOM | 9057 | CA | PRO | X | 118 | 58.043 | 48.617 | −29.797 | 1.00 | 22.67 | X | C |
| ATOM | 9058 | CB | PRO | X | 118 | 58.864 | 49.902 | −29.732 | 1.00 | 20.51 | X | C |
| ATOM | 9059 | CG | PRO | X | 118 | 60.261 | 49.364 | −29.543 | 1.00 | 19.54 | X | C |
| ATOM | 9060 | C | PRO | X | 118 | 58.095 | 47.994 | −31.187 | 1.00 | 23.54 | X | C |
| ATOM | 9061 | O | PRO | X | 118 | 58.964 | 47.188 | −31.483 | 1.00 | 31.48 | X | O |
| ATOM | 9062 | N | PRO | X | 119 | 57.165 | 48.370 | −32.063 | 1.00 | 24.40 | X | N |
| ATOM | 9063 | CD | PRO | X | 119 | 55.982 | 49.222 | −31.832 | 1.00 | 18.98 | X | C |
| ATOM | 9064 | CA | PRO | X | 119 | 57.157 | 47.812 | −33.416 | 1.00 | 22.64 | X | C |
| ATOM | 9065 | CB | PRO | X | 119 | 55.732 | 48.087 | −33.856 | 1.00 | 19.98 | X | C |
| ATOM | 9066 | CG | PRO | X | 119 | 55.486 | 49.460 | −33.236 | 1.00 | 17.64 | X | C |
| ATOM | 9067 | C | PRO | X | 119 | 58.101 | 48.519 | −34.371 | 1.00 | 22.69 | X | C |
| ATOM | 9068 | O | PRO | X | 119 | 58.507 | 49.647 | −34.114 | 1.00 | 27.00 | X | O |
| ATOM | 9069 | N | SER | X | 120 | 58.487 | 47.823 | −35.439 | 1.00 | 20.28 | X | N |
| ATOM | 9070 | CA | SER | X | 120 | 59.277 | 48.425 | −36.510 | 1.00 | 15.48 | X | C |
| ATOM | 9071 | CB | SER | X | 120 | 60.330 | 47.473 | −37.087 | 1.00 | 18.91 | X | C |
| ATOM | 9072 | OG | SER | X | 120 | 61.255 | 47.041 | −36.111 | 1.00 | 20.59 | X | O |
| ATOM | 9073 | C | SER | X | 120 | 58.166 | 48.625 | −37.537 | 1.00 | 17.11 | X | C |
| ATOM | 9074 | O | SER | X | 120 | 57.376 | 47.717 | −37.792 | 1.00 | 23.93 | X | O |
| ATOM | 9075 | N | VAL | X | 121 | 58.067 | 49.826 | −38.076 | 1.00 | 17.21 | X | N |
| ATOM | 9076 | CA | VAL | X | 121 | 57.044 | 50.158 | −39.039 | 1.00 | 13.33 | X | C |
| ATOM | 9077 | CB | VAL | X | 121 | 56.400 | 51.475 | −38.632 | 1.00 | 10.20 | X | C |
| ATOM | 9078 | CG1 | VAL | X | 121 | 55.342 | 51.898 | −39.632 | 1.00 | 9.48 | X | C |
| ATOM | 9079 | CG2 | VAL | X | 121 | 55.793 | 51.317 | −37.252 | 1.00 | 10.81 | X | C |
| ATOM | 9080 | C | VAL | X | 121 | 57.624 | 50.233 | −40.449 | 1.00 | 18.83 | X | C |
| ATOM | 9081 | O | VAL | X | 121 | 58.533 | 51.024 | −40.713 | 1.00 | 17.01 | X | O |
| ATOM | 9082 | N | TYR | X | 122 | 57.088 | 49.403 | −41.346 | 1.00 | 19.98 | X | N |
| ATOM | 9083 | CA | TYR | X | 122 | 57.549 | 49.337 | −42.734 | 1.00 | 21.24 | X | C |
| ATOM | 9084 | CB | TYR | X | 122 | 58.020 | 47.922 | −43.067 | 1.00 | 19.14 | X | C |
| ATOM | 9085 | CG | TYR | X | 122 | 59.106 | 47.415 | −42.150 | 1.00 | 21.22 | X | C |
| ATOM | 9086 | CD1 | TYR | X | 122 | 60.422 | 47.912 | −42.224 | 1.00 | 25.86 | X | C |
| ATOM | 9087 | CE1 | TYR | X | 122 | 61.421 | 47.439 | −41.362 | 1.00 | 15.60 | X | C |
| ATOM | 9088 | CD2 | TYR | X | 122 | 58.829 | 46.443 | −41.206 | 1.00 | 21.74 | X | C |
| ATOM | 9089 | CE2 | TYR | X | 122 | 59.816 | 45.964 | −40.354 | 1.00 | 21.73 | X | C |
| ATOM | 9090 | CZ | TYR | X | 122 | 61.104 | 46.463 | −40.430 | 1.00 | 21.62 | X | C |
| ATOM | 9091 | OH | TYR | X | 122 | 62.045 | 45.971 | −39.539 | 1.00 | 26.49 | X | O |
| ATOM | 9092 | C | TYR | X | 122 | 56.475 | 49.779 | −43.716 | 1.00 | 21.19 | X | C |
| ATOM | 9093 | O | TYR | X | 122 | 55.310 | 49.407 | −43.599 | 1.00 | 23.91 | X | O |
| ATOM | 9094 | N | PRO | X | 123 | 56.857 | 50.574 | −44.719 | 1.00 | 24.34 | X | N |
| ATOM | 9095 | CD | PRO | X | 123 | 58.201 | 51.081 | −45.041 | 1.00 | 21.92 | X | C |
| ATOM | 9096 | CA | PRO | X | 123 | 55.891 | 51.046 | −45.697 | 1.00 | 20.63 | X | C |
| ATOM | 9097 | CB | PRO | X | 123 | 56.628 | 52.181 | −46.370 | 1.00 | 17.38 | X | C |
| ATOM | 9098 | CG | PRO | X | 123 | 58.003 | 51.669 | −46.422 | 1.00 | 20.48 | X | C |
| ATOM | 9099 | C | PRO | X | 123 | 55.446 | 49.967 | −46.692 | 1.00 | 25.81 | X | C |
| ATOM | 9100 | O | PRO | X | 123 | 56.222 | 49.088 | −47.071 | 1.00 | 24.05 | X | O |
| ATOM | 9101 | N | LEU | X | 124 | 54.160 | 49.999 | −47.020 | 1.00 | 24.57 | X | N |
| ATOM | 9102 | CA | LEU | X | 124 | 53.569 | 49.085 | −47.970 | 1.00 | 21.05 | X | C |
| ATOM | 9103 | CB | LEU | X | 124 | 52.325 | 48.417 | −47.393 | 1.00 | 24.00 | X | C |
| ATOM | 9104 | CG | LEU | X | 124 | 52.514 | 47.585 | −46.127 | 1.00 | 28.20 | X | C |
| ATOM | 9105 | CD1 | LEU | X | 124 | 51.221 | 46.888 | −45.794 | 1.00 | 26.09 | X | C |
| ATOM | 9106 | CD2 | LEU | X | 124 | 53.630 | 46.579 | −46.309 | 1.00 | 25.77 | X | C |
| ATOM | 9107 | C | LEU | X | 124 | 53.181 | 49.966 | −49.140 | 1.00 | 26.02 | X | C |
| ATOM | 9108 | O | LEU | X | 124 | 52.204 | 50.720 | −49.065 | 1.00 | 24.50 | X | O |
| ATOM | 9109 | N | ALA | X | 125 | 54.022 | 49.937 | −50.170 | 1.00 | 24.80 | X | N |
| ATOM | 9110 | CA | ALA | X | 125 | 53.830 | 50.704 | −51.388 | 1.00 | 25.20 | X | C |
| ATOM | 9111 | CB | ALA | X | 125 | 54.994 | 51.615 | −51.607 | 1.00 | 19.74 | X | C |
| ATOM | 9112 | C | ALA | X | 125 | 53.729 | 49.715 | −52.539 | 1.00 | 29.69 | X | C |
| ATOM | 9113 | O | ALA | X | 125 | 54.423 | 48.695 | −52.553 | 1.00 | 29.50 | X | O |
| ATOM | 9114 | N | PRO | X | 126 | 52.903 | 50.031 | −53.544 | 1.00 | 33.53 | X | N |
| ATOM | 9115 | CD | PRO | X | 126 | 52.085 | 51.258 | −53.635 | 1.00 | 32.97 | X | C |
| ATOM | 9116 | CA | PRO | X | 126 | 52.689 | 49.180 | −54.715 | 1.00 | 38.34 | X | C |
| ATOM | 9117 | CB | PRO | X | 126 | 51.309 | 49.612 | −55.160 | 1.00 | 35.53 | X | C |
| ATOM | 9118 | CG | PRO | X | 126 | 51.392 | 51.105 | −54.983 | 1.00 | 32.39 | X | C |
| ATOM | 9119 | C | PRO | X | 126 | 53.661 | 49.463 | −55.836 | 1.00 | 47.79 | X | C |
| ATOM | 9120 | O | PRO | X | 126 | 54.168 | 50.579 | −55.943 | 1.00 | 57.04 | X | O |
| ATOM | 9121 | N | VAL | X | 127 | 53.940 | 48.450 | −56.654 | 1.00 | 57.89 | X | N |
| ATOM | 9122 | CA | VAL | X | 127 | 54.780 | 48.629 | −57.844 | 1.00 | 64.73 | X | C |
| ATOM | 9123 | CB | VAL | X | 127 | 56.299 | 48.893 | −57.554 | 1.00 | 66.24 | X | C |
| ATOM | 9124 | CG1 | VAL | X | 127 | 57.044 | 47.608 | −57.165 | 1.00 | 67.81 | X | C |
| ATOM | 9125 | CG2 | VAL | X | 127 | 56.947 | 49.564 | −58.778 | 1.00 | 63.60 | X | C |
| ATOM | 9126 | C | VAL | X | 127 | 54.567 | 47.506 | −58.850 | 1.00 | 68.13 | X | C |
| ATOM | 9127 | O | VAL | X | 127 | 55.510 | 46.877 | −59.342 | 1.00 | 69.32 | X | O |
| ATOM | 9128 | N | CYS | X | 128 | 53.294 | 47.260 | −59.146 | 1.00 | 75.87 | X | N |
| ATOM | 9129 | CA | CYS | X | 128 | 52.910 | 46.238 | −60.114 | 1.00 | 81.28 | X | C |
| ATOM | 9130 | CB | CYS | X | 128 | 51.450 | 45.820 | −59.883 | 1.00 | 82.11 | X | C |
| ATOM | 9131 | SG | CYS | X | 128 | 50.962 | 44.218 | −60.610 | 1.00 | 89.80 | X | S |
| ATOM | 9132 | C | CYS | X | 128 | 53.093 | 46.868 | −61.506 | 1.00 | 82.76 | X | C |
| ATOM | 9133 | O | CYS | X | 128 | 53.168 | 46.172 | −62.526 | 1.00 | 82.65 | X | O |
| ATOM | 9134 | N | GLY | X | 129 | 53.210 | 48.195 | −61.520 | 1.00 | 82.84 | X | N |
| ATOM | 9135 | CA | GLY | X | 129 | 53.396 | 48.925 | −62.758 | 1.00 | 82.27 | X | C |
| ATOM | 9136 | C | GLY | X | 129 | 52.901 | 50.352 | −62.633 | 1.00 | 81.42 | X | C |
| ATOM | 9137 | O | GLY | X | 129 | 53.452 | 51.150 | −61.864 | 1.00 | 79.99 | X | O |
| ATOM | 9138 | N | ASP | X | 130 | 51.840 | 50.654 | −63.378 | 1.00 | 80.81 | X | N |
| ATOM | 9139 | CA | ASP | X | 130 | 51.218 | 51.979 | −63.402 | 1.00 | 82.28 | X | C |
| ATOM | 9140 | CB | ASP | X | 130 | 50.986 | 52.417 | −64.861 | 1.00 | 79.34 | X | C |
| ATOM | 9141 | CG | ASP | X | 130 | 49.976 | 51.517 | −65.601 | 1.00 | 78.77 | X | C |
| ATOM | 9142 | OD1 | ASP | X | 130 | 50.301 | 50.338 | −65.869 | 1.00 | 78.80 | X | O |
| ATOM | 9143 | OD2 | ASP | X | 130 | 48.856 | 51.989 | −65.915 | 1.00 | 78.52 | X | O |
| ATOM | 9144 | C | ASP | X | 130 | 49.873 | 51.974 | −62.663 | 1.00 | 84.09 | X | C |
| ATOM | 9145 | O | ASP | X | 130 | 49.548 | 51.031 | −61.922 | 1.00 | 83.55 | X | O |
| ATOM | 9146 | N | THR | X | 131 | 49.095 | 53.037 | −62.875 | 1.00 | 85.34 | X | N |
| ATOM | 9147 | CA | THR | X | 131 | 47.771 | 53.146 | −62.270 | 1.00 | 86.56 | X | C |
| ATOM | 9148 | CB | THR | X | 131 | 47.336 | 54.633 | −62.050 | 1.00 | 87.58 | X | C |
| ATOM | 9149 | OG1 | THR | X | 131 | 47.653 | 55.420 | −63.209 | 1.00 | 87.00 | X | O |
| ATOM | 9150 | CG2 | THR | X | 131 | 48.033 | 55.223 | −60.815 | 1.00 | 86.95 | X | C |
| ATOM | 9151 | C | THR | X | 131 | 46.722 | 52.370 | −63.094 | 1.00 | 86.94 | X | C |
| ATOM | 9152 | O | THR | X | 131 | 46.422 | 52.718 | −64.252 | 1.00 | 87.21 | X | O |
| ATOM | 9153 | N | THR | X | 132 | 46.242 | 51.268 | −62.506 | 1.00 | 84.93 | X | N |
| ATOM | 9154 | CA | THR | X | 132 | 45.226 | 50.394 | −63.114 | 1.00 | 80.74 | X | C |
| ATOM | 9155 | CB | THR | X | 132 | 45.401 | 48.901 | −62.672 | 1.00 | 79.20 | X | C |
| ATOM | 9156 | OG1 | THR | X | 132 | 44.491 | 48.072 | −63.409 | 1.00 | 70.67 | X | O |
| ATOM | 9157 | CG2 | THR | X | 132 | 45.125 | 48.728 | −61.160 | 1.00 | 73.47 | X | C |
| ATOM | 9158 | C | THR | X | 132 | 43.814 | 50.859 | −62.720 | 1.00 | 83.22 | X | C |
| ATOM | 9159 | O | THR | X | 132 | 42.816 | 50.171 | −62.980 | 1.00 | 85.65 | X | O |
| ATOM | 9160 | N | GLY | X | 133 | 43.757 | 52.021 | −62.071 | 1.00 | 81.10 | X | N |
| ATOM | 9161 | CA | GLY | X | 133 | 42.504 | 52.608 | −61.630 | 1.00 | 75.15 | X | C |
| ATOM | 9162 | C | GLY | X | 133 | 42.795 | 54.018 | −61.147 | 1.00 | 73.68 | X | C |
| ATOM | 9163 | O | GLY | X | 133 | 43.960 | 54.429 | −61.078 | 1.00 | 73.66 | X | O |
| ATOM | 9164 | N | SER | X | 134 | 41.747 | 54.783 | −60.856 | 1.00 | 68.40 | X | N |
| ATOM | 9165 | CA | SER | X | 134 | 41.935 | 56.144 | −60.370 | 1.00 | 63.25 | X | C |
| ATOM | 9166 | CB | SER | X | 134 | 40.681 | 56.985 | −60.629 | 1.00 | 65.01 | X | C |
| ATOM | 9167 | OG | SER | X | 134 | 39.569 | 56.498 | −59.901 | 1.00 | 60.95 | X | O |
| ATOM | 9168 | C | SER | X | 134 | 42.258 | 56.113 | −58.874 | 1.00 | 60.94 | X | C |
| ATOM | 9169 | O | SER | X | 134 | 42.549 | 57.145 | −58.269 | 1.00 | 58.29 | X | O |
| ATOM | 9170 | N | SER | X | 135 | 42.219 | 54.916 | −58.294 | 1.00 | 56.29 | X | N |
| ATOM | 9171 | CA | SER | X | 135 | 42.498 | 54.726 | −56.878 | 1.00 | 49.78 | X | C |
| ATOM | 9172 | CB | SER | X | 135 | 41.316 | 54.005 | −56.208 | 1.00 | 49.03 | X | C |
| ATOM | 9173 | OG | SER | X | 135 | 40.138 | 54.811 | −56.197 | 1.00 | 44.27 | X | O |
| ATOM | 9174 | C | SER | X | 135 | 43.803 | 53.955 | −56.660 | 1.00 | 48.26 | X | C |
| ATOM | 9175 | O | SER | X | 135 | 44.239 | 53.199 | −57.534 | 1.00 | 50.95 | X | O |
| ATOM | 9176 | N | VAL | X | 136 | 44.469 | 54.226 | −55.537 | 1.00 | 43.63 | X | N |
| ATOM | 9177 | CA | VAL | X | 136 | 45.714 | 53.538 | −55.170 | 1.00 | 35.67 | X | C |
| ATOM | 9178 | CB | VAL | X | 136 | 46.992 | 54.341 | −55.555 | 1.00 | 35.58 | X | C |
| ATOM | 9179 | CG1 | VAL | X | 136 | 47.068 | 55.651 | −54.785 | 1.00 | 35.48 | X | C |
| ATOM | 9180 | CG2 | VAL | X | 136 | 48.242 | 53.497 | −55.316 | 1.00 | 29.53 | X | C |
| ATOM | 9181 | C | VAL | X | 136 | 45.671 | 53.221 | −53.670 | 1.00 | 36.06 | X | C |
| ATOM | 9182 | O | VAL | X | 136 | 45.165 | 54.008 | −52.853 | 1.00 | 29.70 | X | O |
| ATOM | 9183 | N | THR | X | 137 | 46.122 | 52.022 | −53.324 | 1.00 | 32.04 | X | N |
| ATOM | 9184 | CA | THR | X | 137 | 46.101 | 51.572 | −51.947 | 1.00 | 30.38 | X | C |
| ATOM | 9185 | CB | THR | X | 137 | 45.329 | 50.241 | −51.839 | 1.00 | 30.27 | X | C |
| ATOM | 9186 | OG1 | THR | X | 137 | 44.008 | 50.432 | −52.354 | 1.00 | 34.77 | X | O |
| ATOM | 9187 | CG2 | THR | X | 137 | 45.225 | 49.774 | −50.405 | 1.00 | 30.94 | X | C |
| ATOM | 9188 | C | THR | X | 137 | 47.504 | 51.450 | −51.375 | 1.00 | 27.55 | X | C |
| ATOM | 9189 | O | THR | X | 137 | 48.353 | 50.771 | −51.933 | 1.00 | 33.67 | X | O |
| ATOM | 9190 | N | LEU | X | 138 | 47.741 | 52.164 | −50.278 | 1.00 | 30.57 | X | N |
| ATOM | 9191 | CA | LEU | X | 138 | 49.028 | 52.165 | −49.578 | 1.00 | 24.66 | X | C |
| ATOM | 9192 | CB | LEU | X | 138 | 49.569 | 53.588 | −49.395 | 1.00 | 23.60 | X | C |
| ATOM | 9193 | CG | LEU | X | 138 | 49.933 | 54.552 | −50.511 | 1.00 | 17.23 | X | C |
| ATOM | 9194 | CD1 | LEU | X | 138 | 50.431 | 53.771 | −51.710 | 1.00 | 22.25 | X | C |
| ATOM | 9195 | CD2 | LEU | X | 138 | 48.747 | 55.405 | −50.845 | 1.00 | 25.42 | X | C |
| ATOM | 9196 | C | LEU | X | 138 | 48.795 | 51.612 | −48.186 | 1.00 | 23.38 | X | C |
| ATOM | 9197 | O | LEU | X | 138 | 47.675 | 51.627 | −47.699 | 1.00 | 21.15 | X | O |
| ATOM | 9198 | N | GLY | X | 139 | 49.866 | 51.182 | −47.528 | 1.00 | 25.04 | X | N |
| ATOM | 9199 | CA | GLY | X | 139 | 49.741 | 50.666 | −46.176 | 1.00 | 21.94 | X | C |
| ATOM | 9200 | C | GLY | X | 139 | 51.036 | 50.724 | −45.372 | 1.00 | 27.28 | X | C |
| ATOM | 9201 | O | GLY | X | 139 | 52.053 | 51.230 | −45.849 | 1.00 | 21.67 | X | O |
| ATOM | 9202 | N | CYS | X | 140 | 51.001 | 50.246 | −44.132 | 0.50 | 24.59 | X | N |
| ATOM | 9203 | CA | CYS | X | 140 | 52.197 | 50.206 | −43.294 | 0.50 | 24.02 | X | C |
| ATOM | 9204 | C | CYS | X | 140 | 52.145 | 48.976 | −42.415 | 0.50 | 22.56 | X | C |
| ATOM | 9205 | O | CYS | X | 140 | 51.154 | 48.723 | −41.730 | 0.50 | 23.91 | X | O |
| ATOM | 9206 | CB | CYS | X | 140 | 52.403 | 51.485 | −42.464 | 0.50 | 25.75 | X | C |
| ATOM | 9207 | SG | CYS | X | 140 | 51.175 | 51.904 | −41.195 | 0.50 | 26.77 | X | S |
| ATOM | 9208 | N | LEU | X | 141 | 53.220 | 48.199 | −42.490 | 1.00 | 16.53 | X | N |
| ATOM | 9209 | CA | LEU | X | 141 | 53.376 | 46.956 | −41.772 | 1.00 | 19.39 | X | C |
| ATOM | 9210 | CB | LEU | X | 141 | 54.355 | 46.069 | −42.551 | 1.00 | 18.37 | X | C |
| ATOM | 9211 | CG | LEU | X | 141 | 54.600 | 44.654 | −42.040 | 1.00 | 18.68 | X | C |
| ATOM | 9212 | CD1 | LEU | X | 141 | 53.312 | 43.854 | −42.096 | 1.00 | 21.31 | X | C |
| ATOM | 9213 | CD2 | LEU | X | 141 | 55.653 | 44.005 | −42.885 | 1.00 | 21.42 | X | C |
| ATOM | 9214 | C | LEU | X | 141 | 53.919 | 47.284 | −40.369 | 1.00 | 23.06 | X | C |
| ATOM | 9215 | O | LEU | X | 141 | 55.029 | 47.796 | −40.220 | 1.00 | 18.83 | X | O |
| ATOM | 9216 | N | VAL | X | 142 | 53.093 | 47.089 | −39.350 | 1.00 | 23.07 | X | N |
| ATOM | 9217 | CA | VAL | X | 142 | 53.520 | 47.348 | −37.979 | 1.00 | 18.64 | X | C |
| ATOM | 9218 | CB | VAL | X | 142 | 52.389 | 47.904 | −37.172 | 1.00 | 16.97 | X | C |
| ATOM | 9219 | CG1 | VAL | X | 142 | 52.853 | 48.186 | −35.762 | 1.00 | 17.70 | X | C |
| ATOM | 9220 | CG2 | VAL | X | 142 | 51.883 | 49.180 | −37.843 | 1.00 | 14.67 | X | C |
| ATOM | 9221 | C | VAL | X | 142 | 53.982 | 45.999 | −37.450 | 1.00 | 18.40 | X | C |
| ATOM | 9222 | O | VAL | X | 142 | 53.189 | 45.192 | −36.975 | 1.00 | 21.93 | X | O |
| ATOM | 9223 | N | LYS | X | 143 | 55.294 | 45.795 | −37.496 | 1.00 | 17.38 | X | N |
| ATOM | 9224 | CA | LYS | X | 143 | 55.897 | 44.519 | −37.156 | 1.00 | 19.72 | X | C |
| ATOM | 9225 | CB | LYS | X | 143 | 56.769 | 44.096 | −38.352 | 1.00 | 15.64 | X | C |
| ATOM | 9226 | CG | LYS | X | 143 | 57.436 | 42.732 | −38.240 | 1.00 | 15.19 | X | C |
| ATOM | 9227 | CD | LYS | X | 143 | 58.365 | 42.482 | −39.432 | 1.00 | 12.09 | X | C |
| ATOM | 9228 | CE | LYS | X | 143 | 59.044 | 41.133 | −39.312 | 1.00 | 13.18 | X | C |
| ATOM | 9229 | NZ | LYS | X | 143 | 59.778 | 40.790 | −40.554 | 1.00 | 14.89 | X | N |
| ATOM | 9230 | C | LYS | X | 143 | 56.688 | 44.336 | −35.864 | 1.00 | 23.51 | X | C |
| ATOM | 9231 | O | LYS | X | 143 | 57.535 | 45.153 | −35.508 | 1.00 | 35.65 | X | O |
| ATOM | 9232 | N | GLY | X | 144 | 56.449 | 43.197 | −35.223 | 1.00 | 20.44 | X | N |
| ATOM | 9233 | CA | GLY | X | 144 | 57.146 | 42.820 | −34.008 | 1.00 | 26.55 | X | C |
| ATOM | 9234 | C | GLY | X | 144 | 56.976 | 43.638 | −32.746 | 1.00 | 26.22 | X | C |
| ATOM | 9235 | O | GLY | X | 144 | 57.967 | 44.086 | −32.189 | 1.00 | 29.25 | X | O |
| ATOM | 9236 | N | TYR | X | 145 | 55.753 | 43.788 | −32.255 | 1.00 | 23.43 | X | N |
| ATOM | 9237 | CA | TYR | X | 145 | 55.549 | 44.563 | −31.044 | 1.00 | 26.83 | X | C |
| ATOM | 9238 | CB | TYR | X | 145 | 54.659 | 45.764 | −31.317 | 1.00 | 29.00 | X | C |
| ATOM | 9239 | CG | TYR | X | 145 | 53.264 | 45.414 | −31.780 | 1.00 | 35.08 | X | C |
| ATOM | 9240 | CD1 | TYR | X | 145 | 52.989 | 45.221 | −33.128 | 1.00 | 30.64 | X | C |
| ATOM | 9241 | CE1 | TYR | X | 145 | 51.709 | 44.953 | −33.559 | 1.00 | 34.55 | X | C |
| ATOM | 9242 | CD2 | TYR | X | 145 | 52.208 | 45.320 | −30.869 | 1.00 | 31.92 | X | C |
| ATOM | 9243 | CE2 | TYR | X | 145 | 50.929 | 45.048 | −31.289 | 1.00 | 33.31 | X | C |
| ATOM | 9244 | CZ | TYR | X | 145 | 50.681 | 44.866 | −32.639 | 1.00 | 36.64 | X | C |
| ATOM | 9245 | OH | TYR | X | 145 | 49.399 | 44.598 | −33.073 | 1.00 | 39.96 | X | O |
| ATOM | 9246 | C | TYR | X | 145 | 54.947 | 43.727 | −29.940 | 1.00 | 31.79 | X | C |
| ATOM | 9247 | O | TYR | X | 145 | 54.467 | 42.622 | −30.182 | 1.00 | 29.25 | X | O |
| ATOM | 9248 | N | PHE | X | 146 | 54.988 | 44.268 | −28.725 | 1.00 | 33.94 | X | N |
| ATOM | 9249 | CA | PHE | X | 146 | 54.432 | 43.612 | −27.550 | 1.00 | 34.57 | X | C |
| ATOM | 9250 | CB | PHE | X | 146 | 55.277 | 42.414 | −27.135 | 1.00 | 35.10 | X | C |
| ATOM | 9251 | CG | PHE | X | 146 | 54.583 | 41.507 | −26.159 | 1.00 | 40.30 | X | C |
| ATOM | 9252 | CD1 | PHE | X | 146 | 53.766 | 40.470 | −26.615 | 1.00 | 35.94 | X | C |
| ATOM | 9253 | CD2 | PHE | X | 146 | 54.724 | 41.698 | −24.786 | 1.00 | 39.03 | X | C |
| ATOM | 9254 | CE1 | PHE | X | 146 | 53.101 | 39.636 | −25.723 | 1.00 | 38.88 | X | C |
| ATOM | 9255 | CE2 | PHE | X | 146 | 54.058 | 40.865 | −23.881 | 1.00 | 41.23 | X | C |
| ATOM | 9256 | CZ | PHE | X | 146 | 53.247 | 39.834 | −24.350 | 1.00 | 37.82 | X | C |
| ATOM | 9257 | C | PHE | X | 146 | 54.346 | 44.576 | −26.378 | 1.00 | 35.51 | X | C |
| ATOM | 9258 | O | PHE | X | 146 | 55.302 | 45.289 | −26.085 | 1.00 | 42.72 | X | O |
| ATOM | 9259 | N | PRO | X | 147 | 53.209 | 44.580 | −25.667 | 1.00 | 34.13 | X | N |
| ATOM | 9260 | CD | PRO | X | 147 | 53.000 | 45.304 | −24.395 | 1.00 | 32.62 | X | C |
| ATOM | 9261 | CA | PRO | X | 147 | 52.063 | 43.718 | −25.957 | 1.00 | 34.54 | X | C |
| ATOM | 9262 | CB | PRO | X | 147 | 51.475 | 43.499 | −24.565 | 1.00 | 30.74 | X | C |
| ATOM | 9263 | CG | PRO | X | 147 | 51.587 | 44.882 | −23.984 | 1.00 | 30.32 | X | C |
| ATOM | 9264 | C | PRO | X | 147 | 51.070 | 44.478 | −26.831 | 1.00 | 35.19 | X | C |
| ATOM | 9265 | O | PRO | X | 147 | 51.360 | 45.576 | −27.310 | 1.00 | 39.18 | X | O |
| ATOM | 9266 | N | GLU | X | 148 | 49.887 | 43.906 | −27.005 | 1.00 | 30.77 | X | N |
| ATOM | 9267 | CA | GLU | X | 148 | 48.851 | 44.578 | −27.763 | 1.00 | 30.17 | X | C |
| ATOM | 9268 | CB | GLU | X | 148 | 47.696 | 43.618 | −28.015 | 1.00 | 29.62 | X | C |
| ATOM | 9269 | CG | GLU | X | 148 | 48.006 | 42.541 | −28.984 | 1.00 | 37.91 | X | C |
| ATOM | 9270 | CD | GLU | X | 148 | 47.069 | 42.581 | −30.153 | 1.00 | 43.00 | X | C |
| ATOM | 9271 | OE1 | GLU | X | 148 | 47.094 | 43.599 | −30.897 | 1.00 | 43.73 | X | O |
| ATOM | 9272 | OE2 | GLU | X | 148 | 46.309 | 41.603 | −30.315 | 1.00 | 40.13 | X | O |
| ATOM | 9273 | C | GLU | X | 148 | 48.353 | 45.704 | −26.866 | 1.00 | 27.78 | X | C |
| ATOM | 9274 | O | GLU | X | 148 | 48.485 | 45.614 | −25.634 | 1.00 | 24.27 | X | O |
| ATOM | 9275 | N | PRO | X | 149 | 47.812 | 46.791 | −27.459 | 1.00 | 27.29 | X | N |
| ATOM | 9276 | CD | PRO | X | 149 | 46.905 | 47.685 | −26.698 | 1.00 | 23.83 | X | C |
| ATOM | 9277 | CA | PRO | X | 149 | 47.645 | 47.008 | −28.905 | 1.00 | 27.57 | X | C |
| ATOM | 9278 | CB | PRO | X | 149 | 46.187 | 47.439 | −28.989 | 1.00 | 21.38 | X | C |
| ATOM | 9279 | CG | PRO | X | 149 | 46.108 | 48.413 | −27.807 | 1.00 | 23.94 | X | C |
| ATOM | 9280 | C | PRO | X | 149 | 48.567 | 48.127 | −29.468 | 1.00 | 27.59 | X | C |
| ATOM | 9281 | O | PRO | X | 149 | 49.551 | 48.523 | −28.843 | 1.00 | 33.28 | X | O |
| ATOM | 9282 | N | VAL | X | 150 | 48.223 | 48.607 | −30.662 | 1.00 | 28.98 | X | N |
| ATOM | 9283 | CA | VAL | X | 150 | 48.909 | 49.702 | −31.344 | 1.00 | 29.98 | X | C |
| ATOM | 9284 | CB | VAL | X | 150 | 49.864 | 49.230 | −32.451 | 1.00 | 29.60 | X | C |
| ATOM | 9285 | CG1 | VAL | X | 150 | 51.102 | 48.582 | −31.856 | 1.00 | 32.67 | X | C |
| ATOM | 9286 | CG2 | VAL | X | 150 | 49.151 | 48.269 | −33.373 | 1.00 | 28.82 | X | C |
| ATOM | 9287 | C | VAL | X | 150 | 47.785 | 50.401 | −32.044 | 1.00 | 30.41 | X | C |
| ATOM | 9288 | O | VAL | X | 150 | 46.791 | 49.764 | −32.378 | 1.00 | 34.24 | X | O |
| ATOM | 9289 | N | THR | X | 151 | 47.886 | 51.711 | −32.208 | 1.00 | 35.67 | X | N |
| ATOM | 9290 | CA | THR | X | 151 | 46.837 | 52.427 | −32.919 | 1.00 | 35.19 | X | C |
| ATOM | 9291 | CB | THR | X | 151 | 46.293 | 53.654 | −32.139 | 1.00 | 38.25 | X | C |
| ATOM | 9292 | OG1 | THR | X | 151 | 47.348 | 54.597 | −31.921 | 1.00 | 41.09 | X | O |
| ATOM | 9293 | CG2 | THR | X | 151 | 45.679 | 53.221 | −30.809 | 1.00 | 33.18 | X | C |
| ATOM | 9294 | C | THR | X | 151 | 47.471 | 52.865 | −34.218 | 1.00 | 34.70 | X | C |
| ATOM | 9295 | O | THR | X | 151 | 48.694 | 52.852 | −34.345 | 1.00 | 34.24 | X | O |
| ATOM | 9296 | N | LEU | X | 152 | 46.649 | 53.287 | −35.169 | 1.00 | 31.38 | X | N |
| ATOM | 9297 | CA | LEU | X | 152 | 47.176 | 53.693 | −36.442 | 1.00 | 29.80 | X | C |
| ATOM | 9298 | CB | LEU | X | 152 | 47.335 | 52.449 | −37.302 | 1.00 | 35.90 | X | C |
| ATOM | 9299 | CG | LEU | X | 152 | 48.633 | 52.479 | −38.071 | 1.00 | 40.90 | X | C |
| ATOM | 9300 | CD1 | LEU | X | 152 | 49.174 | 51.075 | −38.158 | 1.00 | 49.58 | X | C |
| ATOM | 9301 | CD2 | LEU | X | 152 | 48.420 | 53.125 | −39.427 | 1.00 | 44.12 | X | C |
| ATOM | 9302 | C | LEU | X | 152 | 46.284 | 54.699 | −37.127 | 1.00 | 26.68 | X | C |
| ATOM | 9303 | O | LEU | X | 152 | 45.084 | 54.486 | −37.220 | 1.00 | 31.45 | X | O |
| ATOM | 9304 | N | THR | X | 153 | 46.855 | 55.818 | −37.566 | 1.00 | 26.30 | X | N |
| ATOM | 9305 | CA | THR | X | 153 | 46.070 | 56.825 | −38.279 | 1.00 | 23.70 | X | C |
| ATOM | 9306 | CB | THR | X | 153 | 45.730 | 58.085 | −37.444 | 1.00 | 27.13 | X | C |
| ATOM | 9307 | OG1 | THR | X | 153 | 46.893 | 58.916 | −37.310 | 1.00 | 24.44 | X | O |
| ATOM | 9308 | CG2 | THR | X | 153 | 45.170 | 57.703 | −36.071 | 1.00 | 22.48 | X | C |
| ATOM | 9309 | C | THR | X | 153 | 46.806 | 57.250 | −39.536 | 1.00 | 27.35 | X | C |
| ATOM | 9310 | O | THR | X | 153 | 47.961 | 56.897 | −39.751 | 1.00 | 32.92 | X | O |
| ATOM | 9311 | N | TRP | X | 154 | 46.130 | 58.017 | −40.373 | 1.00 | 26.47 | X | N |
| ATOM | 9312 | CA | TRP | X | 154 | 46.724 | 58.444 | −41.613 | 1.00 | 25.47 | X | C |
| ATOM | 9313 | CB | TRP | X | 154 | 46.045 | 57.705 | −42.754 | 1.00 | 22.86 | X | C |
| ATOM | 9314 | CG | TRP | X | 154 | 46.435 | 56.262 | −42.756 | 1.00 | 20.82 | X | C |
| ATOM | 9315 | CD2 | TRP | X | 154 | 47.567 | 55.688 | −43.423 | 1.00 | 18.61 | X | C |
| ATOM | 9316 | CE2 | TRP | X | 154 | 47.559 | 54.305 | −43.142 | 1.00 | 17.71 | X | C |
| ATOM | 9317 | CE3 | TRP | X | 154 | 48.591 | 56.214 | −44.233 | 1.00 | 16.05 | X | C |
| ATOM | 9318 | CD1 | TRP | X | 154 | 45.806 | 55.242 | −42.115 | 1.00 | 19.60 | X | C |
| ATOM | 9319 | NE1 | TRP | X | 154 | 46.474 | 54.058 | −42.341 | 1.00 | 21.68 | X | N |
| ATOM | 9320 | CZ2 | TRP | X | 154 | 48.538 | 53.426 | −43.640 | 1.00 | 20.46 | X | C |
| ATOM | 9321 | CZ3 | TRP | X | 154 | 49.575 | 55.340 | −44.732 | 1.00 | 16.01 | X | C |
| ATOM | 9322 | CH2 | TRP | X | 154 | 49.535 | 53.957 | −44.431 | 1.00 | 17.34 | X | C |
| ATOM | 9323 | C | TRP | X | 154 | 46.665 | 59.945 | −41.780 | 1.00 | 23.67 | X | C |
| ATOM | 9324 | O | TRP | X | 154 | 45.596 | 60.534 | −41.737 | 1.00 | 25.47 | X | O |
| ATOM | 9325 | N | ASN | X | 155 | 47.830 | 60.543 | −42.023 | 1.00 | 26.53 | X | N |
| ATOM | 9326 | CA | ASN | X | 155 | 47.982 | 61.985 | −42.161 | 1.00 | 27.66 | X | C |
| ATOM | 9327 | CB | ASN | X | 155 | 47.364 | 62.509 | −43.468 | 1.00 | 27.67 | X | C |
| ATOM | 9328 | CG | ASN | X | 155 | 48.263 | 62.270 | −44.683 | 1.00 | 29.41 | X | C |
| ATOM | 9329 | OD1 | ASN | X | 155 | 49.295 | 61.601 | −44.596 | 1.00 | 34.18 | X | O |
| ATOM | 9330 | ND2 | ASN | X | 155 | 47.888 | 62.845 | −45.812 | 1.00 | 27.24 | X | N |
| ATOM | 9331 | C | ASN | X | 155 | 47.383 | 62.636 | −40.911 | 1.00 | 31.84 | X | C |
| ATOM | 9332 | O | ASN | X | 155 | 46.534 | 63.527 | −40.979 | 1.00 | 37.37 | X | O |
| ATOM | 9333 | N | SER | X | 156 | 47.784 | 62.090 | −39.767 | 1.00 | 29.61 | X | N |
| ATOM | 9334 | CA | SER | X | 156 | 47.376 | 62.563 | −38.448 | 1.00 | 38.64 | X | C |
| ATOM | 9335 | CB | SER | X | 156 | 47.940 | 63.963 | −38.185 | 1.00 | 36.45 | X | C |
| ATOM | 9336 | OG | SER | X | 156 | 49.357 | 63.945 | −38.238 | 1.00 | 39.17 | X | O |
| ATOM | 9337 | C | SER | X | 156 | 45.877 | 62.540 | −38.186 | 1.00 | 41.57 | X | C |
| ATOM | 9338 | O | SER | X | 156 | 45.404 | 63.091 | −37.188 | 1.00 | 41.87 | X | O |
| ATOM | 9339 | N | GLY | X | 157 | 45.142 | 61.852 | −39.054 | 1.00 | 40.54 | X | N |
| ATOM | 9340 | CA | GLY | X | 157 | 43.702 | 61.760 | −38.901 | 1.00 | 32.13 | X | C |
| ATOM | 9341 | C | GLY | X | 157 | 42.978 | 62.391 | −40.071 | 1.00 | 34.01 | X | C |
| ATOM | 9342 | O | GLY | X | 157 | 41.769 | 62.225 | −40.203 | 1.00 | 37.68 | X | O |
| ATOM | 9343 | N | SER | X | 158 | 43.714 | 63.112 | −40.915 | 1.00 | 37.43 | X | N |
| ATOM | 9344 | CA | SER | X | 158 | 43.135 | 63.769 | −42.090 | 1.00 | 46.68 | X | C |
| ATOM | 9345 | CB | SER | X | 158 | 44.192 | 64.570 | −42.855 | 1.00 | 45.71 | X | C |
| ATOM | 9346 | OG | SER | X | 158 | 44.468 | 65.783 | −42.191 | 1.00 | 47.77 | X | O |
| ATOM | 9347 | C | SER | X | 158 | 42.469 | 62.768 | −43.026 | 1.00 | 48.17 | X | C |
| ATOM | 9348 | O | SER | X | 158 | 41.351 | 62.996 | −43.482 | 1.00 | 50.12 | X | O |
| ATOM | 9349 | N | LEU | X | 159 | 43.163 | 61.674 | −43.330 | 1.00 | 44.58 | X | N |
| ATOM | 9350 | CA | LEU | X | 159 | 42.585 | 60.656 | −44.190 | 1.00 | 40.75 | X | C |
| ATOM | 9351 | CB | LEU | X | 159 | 43.655 | 59.959 | −45.023 | 1.00 | 33.40 | X | C |
| ATOM | 9352 | CG | LEU | X | 159 | 44.457 | 60.825 | −45.989 | 1.00 | 36.11 | X | C |
| ATOM | 9353 | CD1 | LEU | X | 159 | 45.443 | 59.937 | −46.719 | 1.00 | 33.50 | X | C |
| ATOM | 9354 | CD2 | LEU | X | 159 | 43.551 | 61.568 | −46.984 | 1.00 | 33.12 | X | C |
| ATOM | 9355 | C | LEU | X | 159 | 41.906 | 59.668 | −43.259 | 1.00 | 40.05 | X | C |
| ATOM | 9356 | O | LEU | X | 159 | 42.577 | 58.913 | −42.559 | 1.00 | 47.34 | X | O |
| ATOM | 9357 | N | SER | X | 160 | 40.579 | 59.728 | −43.207 | 1.00 | 37.92 | X | N |
| ATOM | 9358 | CA | SER | X | 160 | 39.787 | 58.848 | −42.343 | 1.00 | 37.16 | X | C |
| ATOM | 9359 | CB | SER | X | 160 | 38.863 | 59.677 | −41.432 | 1.00 | 39.31 | X | C |
| ATOM | 9360 | OG | SER | X | 160 | 38.100 | 60.622 | −42.169 | 1.00 | 35.65 | X | O |
| ATOM | 9361 | C | SER | X | 160 | 38.966 | 57.834 | −43.141 | 1.00 | 37.69 | X | C |
| ATOM | 9362 | O | SER | X | 160 | 38.658 | 56.745 | −42.659 | 1.00 | 36.16 | X | O |
| ATOM | 9363 | N | SER | X | 161 | 38.607 | 58.216 | −44.357 | 1.00 | 36.08 | X | N |
| ATOM | 9364 | CA | SER | X | 161 | 37.835 | 57.364 | −45.244 | 1.00 | 43.09 | X | C |
| ATOM | 9365 | CB | SER | X | 161 | 37.075 | 58.234 | −46.255 | 1.00 | 47.12 | X | C |
| ATOM | 9366 | OG | SER | X | 161 | 36.267 | 59.202 | −45.598 | 1.00 | 54.55 | X | O |
| ATOM | 9367 | C | SER | X | 161 | 38.764 | 56.410 | −45.992 | 1.00 | 39.36 | X | C |
| ATOM | 9368 | O | SER | X | 161 | 39.879 | 56.787 | −46.356 | 1.00 | 41.82 | X | O |
| ATOM | 9369 | N | GLY | X | 162 | 38.298 | 55.184 | −46.218 | 1.00 | 32.14 | X | N |
| ATOM | 9370 | CA | GLY | X | 162 | 39.091 | 54.202 | −46.941 | 1.00 | 35.62 | X | C |
| ATOM | 9371 | C | GLY | X | 162 | 40.249 | 53.642 | −46.137 | 1.00 | 37.79 | X | C |
| ATOM | 9372 | O | GLY | X | 162 | 41.272 | 53.225 | −46.694 | 1.00 | 40.88 | X | O |
| ATOM | 9373 | N | VAL | X | 163 | 40.095 | 53.659 | −44.820 | 1.00 | 28.00 | X | N |
| ATOM | 9374 | CA | VAL | X | 163 | 41.125 | 53.151 | −43.937 | 1.00 | 26.94 | X | C |
| ATOM | 9375 | CB | VAL | X | 163 | 41.389 | 54.122 | −42.749 | 1.00 | 23.08 | X | C |
| ATOM | 9376 | CG1 | VAL | X | 163 | 42.262 | 53.453 | −41.706 | 1.00 | 17.17 | X | C |
| ATOM | 9377 | CG2 | VAL | X | 163 | 42.056 | 55.373 | −43.242 | 1.00 | 24.73 | X | C |
| ATOM | 9378 | C | VAL | X | 163 | 40.700 | 51.813 | −43.367 | 1.00 | 22.56 | X | C |
| ATOM | 9379 | O | VAL | X | 163 | 39.611 | 51.698 | −42.832 | 1.00 | 30.43 | X | O |
| ATOM | 9380 | N | HIS | X | 164 | 41.583 | 50.821 | −43.458 | 1.00 | 23.51 | X | N |
| ATOM | 9381 | CA | HIS | X | 164 | 41.345 | 49.490 | −42.916 | 1.00 | 16.97 | X | C |
| ATOM | 9382 | CB | HIS | X | 164 | 41.173 | 48.448 | −44.035 | 1.00 | 24.46 | X | C |
| ATOM | 9383 | CG | HIS | X | 164 | 39.895 | 48.575 | −44.802 | 1.00 | 30.46 | X | C |
| ATOM | 9384 | CD2 | HIS | X | 164 | 38.605 | 48.424 | −44.418 | 1.00 | 30.83 | X | C |
| ATOM | 9385 | ND1 | HIS | X | 164 | 39.862 | 48.935 | −46.132 | 1.00 | 30.01 | X | N |
| ATOM | 9386 | CE1 | HIS | X | 164 | 38.604 | 49.010 | −46.533 | 1.00 | 32.75 | X | C |
| ATOM | 9387 | NE2 | HIS | X | 164 | 37.823 | 48.704 | −45.512 | 1.00 | 35.60 | X | N |
| ATOM | 9388 | C | HIS | X | 164 | 42.537 | 49.074 | −42.048 | 1.00 | 23.62 | X | C |
| ATOM | 9389 | O | HIS | X | 164 | 43.688 | 49.077 | −42.500 | 1.00 | 26.67 | X | O |
| ATOM | 9390 | N | THR | X | 165 | 42.269 | 48.738 | −40.795 | 1.00 | 24.20 | X | N |
| ATOM | 9391 | CA | THR | X | 165 | 43.327 | 48.286 | −39.900 | 1.00 | 31.26 | X | C |
| ATOM | 9392 | CB | THR | X | 165 | 43.524 | 49.212 | −38.648 | 1.00 | 32.90 | X | C |
| ATOM | 9393 | OG1 | THR | X | 165 | 44.033 | 50.486 | −39.075 | 1.00 | 34.43 | X | O |
| ATOM | 9394 | CG2 | THR | X | 165 | 44.537 | 48.593 | −37.663 | 1.00 | 30.99 | X | C |
| ATOM | 9395 | C | THR | X | 165 | 42.947 | 46.874 | −39.498 | 1.00 | 30.24 | X | C |
| ATOM | 9396 | O | THR | X | 165 | 41.970 | 46.654 | −38.783 | 1.00 | 39.00 | X | O |
| ATOM | 9397 | N | PHE | X | 166 | 43.736 | 45.928 | −39.985 | 1.00 | 24.24 | X | N |
| ATOM | 9398 | CA | PHE | X | 166 | 43.533 | 44.508 | −39.773 | 1.00 | 26.24 | X | C |
| ATOM | 9399 | CB | PHE | X | 166 | 44.247 | 43.748 | −40.903 | 1.00 | 26.45 | X | C |
| ATOM | 9400 | CG | PHE | X | 166 | 43.834 | 44.201 | −42.283 | 1.00 | 28.19 | X | C |
| ATOM | 9401 | CD1 | PHE | X | 166 | 44.423 | 45.319 | −42.870 | 1.00 | 25.22 | X | C |
| ATOM | 9402 | CD2 | PHE | X | 166 | 42.829 | 43.530 | −42.983 | 1.00 | 31.68 | X | C |
| ATOM | 9403 | CE1 | PHE | X | 166 | 44.021 | 45.773 | −44.136 | 1.00 | 24.90 | X | C |
| ATOM | 9404 | CE2 | PHE | X | 166 | 42.421 | 43.971 | −44.242 | 1.00 | 27.87 | X | C |
| ATOM | 9405 | CZ | PHE | X | 166 | 43.024 | 45.100 | −44.818 | 1.00 | 29.77 | X | C |
| ATOM | 9406 | C | PHE | X | 166 | 43.928 | 43.955 | −38.402 | 1.00 | 30.20 | X | C |
| ATOM | 9407 | O | PHE | X | 166 | 44.596 | 44.625 | −37.613 | 1.00 | 34.14 | X | O |
| ATOM | 9408 | N | PRO | X | 167 | 43.463 | 42.730 | −38.076 | 1.00 | 37.79 | X | N |
| ATOM | 9409 | CD | PRO | X | 167 | 42.522 | 41.867 | −38.820 | 1.00 | 32.89 | X | C |
| ATOM | 9410 | CA | PRO | X | 167 | 43.794 | 42.122 | −36.782 | 1.00 | 34.57 | X | C |
| ATOM | 9411 | CB | PRO | X | 167 | 42.856 | 40.917 | −36.716 | 1.00 | 34.23 | X | C |
| ATOM | 9412 | CG | PRO | X | 167 | 42.712 | 40.529 | −38.139 | 1.00 | 34.62 | X | C |
| ATOM | 9413 | C | PRO | X | 167 | 45.227 | 41.675 | −36.745 | 1.00 | 33.05 | X | C |
| ATOM | 9414 | O | PRO | X | 167 | 45.835 | 41.415 | −37.782 | 1.00 | 32.42 | X | O |
| ATOM | 9415 | N | ALA | X | 168 | 45.757 | 41.552 | −35.538 | 1.00 | 32.81 | X | N |
| ATOM | 9416 | CA | ALA | X | 168 | 47.135 | 41.135 | −35.372 | 1.00 | 30.87 | X | C |
| ATOM | 9417 | CB | ALA | X | 168 | 47.675 | 41.631 | −34.041 | 1.00 | 31.02 | X | C |
| ATOM | 9418 | C | ALA | X | 168 | 47.289 | 39.632 | −35.451 | 1.00 | 31.18 | X | C |
| ATOM | 9419 | O | ALA | X | 168 | 46.355 | 38.883 | −35.131 | 1.00 | 33.02 | X | O |
| ATOM | 9420 | N | VAL | X | 169 | 48.446 | 39.210 | −35.956 | 1.00 | 25.57 | X | N |
| ATOM | 9421 | CA | VAL | X | 169 | 48.814 | 37.798 | −36.023 | 1.00 | 28.06 | X | C |
| ATOM | 9422 | CB | VAL | X | 169 | 49.198 | 37.322 | −37.446 | 1.00 | 26.50 | X | C |
| ATOM | 9423 | CG1 | VAL | X | 169 | 48.098 | 37.663 | −38.434 | 1.00 | 24.39 | X | C |
| ATOM | 9424 | CG2 | VAL | X | 169 | 50.521 | 37.916 | −37.873 | 1.00 | 24.87 | X | C |
| ATOM | 9425 | C | VAL | X | 169 | 50.041 | 37.652 | −35.118 | 1.00 | 34.66 | X | C |
| ATOM | 9426 | O | VAL | X | 169 | 50.781 | 38.618 | −34.897 | 1.00 | 41.20 | X | O |
| ATOM | 9427 | N | LEU | X | 170 | 50.267 | 36.450 | −34.605 | 1.00 | 37.29 | X | N |
| ATOM | 9428 | CA | LEU | X | 170 | 51.389 | 36.214 | −33.720 | 1.00 | 42.39 | X | C |
| ATOM | 9429 | CB | LEU | X | 170 | 50.923 | 35.400 | −32.510 | 1.00 | 35.28 | X | C |
| ATOM | 9430 | CG | LEU | X | 170 | 51.787 | 35.280 | −31.243 | 1.00 | 42.76 | X | C |
| ATOM | 9431 | CD1 | LEU | X | 170 | 52.435 | 36.602 | −30.846 | 1.00 | 30.67 | X | C |
| ATOM | 9432 | CD2 | LEU | X | 170 | 50.896 | 34.767 | −30.104 | 1.00 | 35.92 | X | C |
| ATOM | 9433 | C | LEU | X | 170 | 52.528 | 35.516 | −34.456 | 1.00 | 48.38 | X | C |
| ATOM | 9434 | O | LEU | X | 170 | 52.421 | 34.341 | −34.818 | 1.00 | 53.83 | X | O |
| ATOM | 9435 | N | GLN | X | 171 | 53.589 | 36.274 | −34.738 | 1.00 | 55.47 | X | N |
| ATOM | 9436 | CA | GLN | X | 171 | 54.778 | 35.755 | −35.425 | 1.00 | 56.81 | X | C |
| ATOM | 9437 | CB | GLN | X | 171 | 55.289 | 36.726 | −36.497 | 1.00 | 59.24 | X | C |
| ATOM | 9438 | CG | GLN | X | 171 | 54.384 | 36.979 | −37.688 | 1.00 | 62.57 | X | C |
| ATOM | 9439 | CD | GLN | X | 171 | 54.920 | 38.089 | −38.603 | 1.00 | 64.27 | X | C |
| ATOM | 9440 | OE1 | GLN | X | 171 | 54.156 | 38.734 | −39.326 | 1.00 | 67.80 | X | O |
| ATOM | 9441 | NE2 | GLN | X | 171 | 56.235 | 38.311 | −38.572 | 1.00 | 62.57 | X | N |
| ATOM | 9442 | C | GLN | X | 171 | 55.887 | 35.610 | −34.397 | 1.00 | 59.53 | X | C |
| ATOM | 9443 | O | GLN | X | 171 | 56.399 | 36.609 | −33.876 | 1.00 | 56.75 | X | O |
| ATOM | 9444 | N | SER | X | 172 | 56.253 | 34.366 | −34.113 | 1.00 | 61.82 | X | N |
| ATOM | 9445 | CA | SER | X | 172 | 57.305 | 34.048 | −33.161 | 1.00 | 58.81 | X | C |
| ATOM | 9446 | CB | SER | X | 172 | 58.672 | 34.079 | −33.844 | 1.00 | 62.35 | X | C |
| ATOM | 9447 | OG | SER | X | 172 | 59.656 | 33.495 | −33.002 | 1.00 | 69.85 | X | O |
| ATOM | 9448 | C | SER | X | 172 | 57.317 | 34.929 | −31.917 | 1.00 | 57.42 | X | C |
| ATOM | 9449 | O | SER | X | 172 | 58.238 | 35.736 | −31.716 | 1.00 | 55.39 | X | O |
| ATOM | 9450 | N | ASP | X | 173 | 56.267 | 34.784 | −31.108 | 1.00 | 54.20 | X | N |
| ATOM | 9451 | CA | ASP | X | 173 | 56.113 | 35.504 | −29.839 | 1.00 | 50.26 | X | C |
| ATOM | 9452 | CB | ASP | X | 173 | 57.345 | 35.277 | −28.952 | 1.00 | 56.90 | X | C |
| ATOM | 9453 | CG | ASP | X | 173 | 57.646 | 33.803 | −28.737 | 1.00 | 63.31 | X | C |
| ATOM | 9454 | OD1 | ASP | X | 173 | 58.296 | 33.177 | −29.613 | 1.00 | 65.72 | X | O |
| ATOM | 9455 | OD2 | ASP | X | 173 | 57.229 | 33.269 | −27.688 | 1.00 | 68.23 | X | O |
| ATOM | 9456 | C | ASP | X | 173 | 55.795 | 36.997 | −29.884 | 1.00 | 44.06 | X | C |
| ATOM | 9457 | O | ASP | X | 173 | 55.470 | 37.581 | −28.846 | 1.00 | 46.32 | X | O |
| ATOM | 9458 | N | LEU | X | 174 | 55.905 | 37.618 | −31.056 | 1.00 | 33.49 | X | N |
| ATOM | 9459 | CA | LEU | X | 174 | 55.620 | 39.047 | −31.188 | 1.00 | 29.24 | X | C |
| ATOM | 9460 | CB | LEU | X | 174 | 56.870 | 39.798 | −31.676 | 1.00 | 25.22 | X | C |
| ATOM | 9461 | CG | LEU | X | 174 | 58.158 | 39.656 | −30.828 | 1.00 | 30.49 | X | C |
| ATOM | 9462 | CD1 | LEU | X | 174 | 59.331 | 40.266 | −31.569 | 1.00 | 26.35 | X | C |
| ATOM | 9463 | CD2 | LEU | X | 174 | 58.028 | 40.304 | −29.435 | 1.00 | 20.97 | X | C |
| ATOM | 9464 | C | LEU | X | 174 | 54.423 | 39.258 | −32.123 | 1.00 | 28.34 | X | C |
| ATOM | 9465 | O | LEU | X | 174 | 54.062 | 38.371 | −32.906 | 1.00 | 22.38 | X | O |
| ATOM | 9466 | N | TYR | X | 175 | 53.768 | 40.402 | −31.995 | 1.00 | 24.37 | X | N |
| ATOM | 9467 | CA | TYR | X | 175 | 52.597 | 40.681 | −32.804 | 1.00 | 29.11 | X | C |
| ATOM | 9468 | CB | TYR | X | 175 | 51.516 | 41.375 | −31.968 | 1.00 | 26.13 | X | C |
| ATOM | 9469 | CG | TYR | X | 175 | 50.872 | 40.515 | −30.895 | 1.00 | 33.58 | X | C |
| ATOM | 9470 | CD1 | TYR | X | 175 | 49.658 | 39.858 | −31.129 | 1.00 | 36.53 | X | C |
| ATOM | 9471 | CE1 | TYR | X | 175 | 49.030 | 39.120 | −30.132 | 1.00 | 31.81 | X | C |
| ATOM | 9472 | CD2 | TYR | X | 175 | 51.440 | 40.400 | −29.626 | 1.00 | 33.34 | X | C |
| ATOM | 9473 | CE2 | TYR | X | 175 | 50.819 | 39.666 | −28.625 | 1.00 | 32.94 | X | C |
| ATOM | 9474 | CZ | TYR | X | 175 | 49.614 | 39.031 | −28.882 | 1.00 | 38.85 | X | C |
| ATOM | 9475 | OH | TYR | X | 175 | 48.987 | 38.328 | −27.877 | 1.00 | 41.32 | X | O |
| ATOM | 9476 | C | TYR | X | 175 | 52.913 | 41.525 | −34.032 | 1.00 | 34.37 | X | C |
| ATOM | 9477 | O | TYR | X | 175 | 53.847 | 42.333 | −34.022 | 1.00 | 33.61 | X | O |
| ATOM | 9478 | N | THR | X | 176 | 52.145 | 41.287 | −35.098 | 1.00 | 29.66 | X | N |
| ATOM | 9479 | CA | THR | X | 176 | 52.258 | 42.033 | −36.350 | 1.00 | 28.49 | X | C |
| ATOM | 9480 | CB | THR | X | 176 | 53.016 | 41.260 | −37.452 | 1.00 | 27.23 | X | C |
| ATOM | 9481 | OG1 | THR | X | 176 | 54.370 | 41.048 | −37.051 | 1.00 | 31.59 | X | O |
| ATOM | 9482 | CG2 | THR | X | 17.6 | 53.023 | 42.047 | −38.753 | 1.00 | 26.42 | X | C |
| ATOM | 9483 | C | THR | X | 176 | 50.833 | 42.335 | −36.821 | 1.00 | 28.09 | X | C |
| ATOM | 9484 | O | THR | X | 176 | 49.905 | 41.545 | −36.628 | 1.00 | 30.13 | X | O |
| ATOM | 9485 | N | LEU | X | 177 | 50.681 | 43.462 | −37.490 | 1.00 | 22.51 | X | N |
| ATOM | 9486 | CA | LEU | X | 177 | 49.386 | 43.908 | −37.957 | 1.00 | 21.58 | X | C |
| ATOM | 9487 | CB | LEU | X | 177 | 48.704 | 44.647 | −36.798 | 1.00 | 14.40 | X | C |
| ATOM | 9488 | CG | LEU | X | 177 | 47.806 | 45.877 | −36.871 | 1.00 | 19.58 | X | C |
| ATOM | 9489 | CD1 | LEU | X | 177 | 47.260 | 46.121 | −35.474 | 1.00 | 16.01 | X | C |
| ATOM | 9490 | CD2 | LEU | X | 177 | 48.518 | 47.110 | −37.378 | 1.00 | 10.03 | X | C |
| ATOM | 9491 | C | LEU | X | 177 | 49.638 | 44.818 | −39.142 | 1.00 | 22.61 | X | C |
| ATOM | 9492 | O | LEU | X | 177 | 50.770 | 45.236 | −39.379 | 1.00 | 28.69 | X | O |
| ATOM | 9493 | N | SER | X | 178 | 48.588 | 45.133 | −39.887 | 1.00 | 22.10 | X | N |
| ATOM | 9494 | CA | SER | X | 178 | 48.736 | 46.006 | −41.035 | 1.00 | 18.67 | X | C |
| ATOM | 9495 | CB | SER | X | 178 | 48.845 | 45.189 | −42.317 | 1.00 | 27.31 | X | C |
| ATOM | 9496 | OG | SER | X | 178 | 50.001 | 44.375 | −42.300 | 1.00 | 37.23 | X | O |
| ATOM | 9497 | C | SER | X | 178 | 47.547 | 46.924 | −41.160 | 1.00 | 20.80 | X | C |
| ATOM | 9498 | O | SER | X | 178 | 46.440 | 46.585 | −40.763 | 1.00 | 21.91 | X | O |
| ATOM | 9499 | N | SER | X | 179 | 47.797 | 48.099 | −41.706 | 1.00 | 15.70 | X | N |
| ATOM | 9500 | CA | SER | X | 179 | 46.764 | 49.072 | −41.937 | 1.00 | 16.02 | X | C |
| ATOM | 9501 | CB | SER | X | 179 | 46.909 | 50.253 | −40.979 | 1.00 | 15.51 | X | C |
| ATOM | 9502 | OG | SER | X | 179 | 45.999 | 51.292 | −41.320 | 1.00 | 26.31 | X | O |
| ATOM | 9503 | C | SER | X | 179 | 46.970 | 49.555 | −43.361 | 1.00 | 15.16 | X | C |
| ATOM | 9504 | O | SER | X | 179 | 48.093 | 49.559 | −43.867 | 1.00 | 19.99 | X | O |
| ATOM | 9505 | N | SER | X | 180 | 45.890 | 49.961 | −44.006 | 1.00 | 15.72 | X | N |
| ATOM | 9506 | CA | SER | X | 180 | 45.979 | 50.472 | −45.361 | 1.00 | 23.53 | X | C |
| ATOM | 9507 | CB | SER | X | 180 | 45.642 | 49.377 | −46.385 | 1.00 | 16.22 | X | C |
| ATOM | 9508 | OG | SER | X | 180 | 44.328 | 48.890 | −46.184 | 1.00 | 31.01 | X | O |
| ATOM | 9509 | C | SER | X | 180 | 45.035 | 51.642 | −45.523 | 1.00 | 19.44 | X | C |
| ATOM | 9510 | O | SER | X | 180 | 44.137 | 51.850 | −44.715 | 1.00 | 22.50 | X | O |
| ATOM | 9511 | N | VAL | X | 181 | 45.269 | 52.421 | −46.563 | 1.00 | 25.04 | X | N |
| ATOM | 9512 | CA | VAL | X | 181 | 44.435 | 53.564 | −46.882 | 1.00 | 22.42 | X | C |
| ATOM | 9513 | CB | VAL | X | 181 | 45.024 | 54.904 | −46.344 | 1.00 | 18.54 | X | C |
| ATOM | 9514 | CG1 | VAL | X | 181 | 46.508 | 55.003 | −46.620 | 1.00 | 14.24 | X | C |
| ATOM | 9515 | CG2 | VAL | X | 181 | 44.305 | 56.077 | −46.976 | 1.00 | 16.83 | X | C |
| ATOM | 9516 | C | VAL | X | 181 | 44.343 | 53.603 | −48.389 | 1.00 | 26.71 | X | C |
| ATOM | 9517 | O | VAL | X | 181 | 45.317 | 53.299 | −49.087 | 1.00 | 27.08 | X | O |
| ATOM | 9518 | N | THR | X | 182 | 43.151 | 53.896 | −48.895 | 1.00 | 31.82 | X | N |
| ATOM | 9519 | CA | THR | X | 182 | 42.961 | 53.977 | −50.331 | 1.00 | 30.73 | X | C |
| ATOM | 9520 | CB | THR | X | 182 | 41.828 | 53.044 | −50.790 | 1.00 | 32.91 | X | C |
| ATOM | 9521 | OG1 | THR | X | 182 | 42.153 | 51.693 | −50.430 | 1.00 | 35.98 | X | O |
| ATOM | 9522 | CG2 | THR | X | 182 | 41.669 | 53.110 | −52.299 | 1.00 | 32.60 | X | C |
| ATOM | 9523 | C | THR | X | 182 | 42.667 | 55.425 | −50.680 | 1.00 | 30.25 | X | C |
| ATOM | 9524 | O | THR | X | 182 | 41.715 | 56.006 | −50.171 | 1.00 | 37.47 | X | O |
| ATOM | 9525 | N | VAL | X | 183 | 43.532 | 56.020 | −51.497 | 1.00 | 31.45 | X | N |
| ATOM | 9526 | CA | VAL | X | 183 | 43.382 | 57.413 | −51.918 | 1.00 | 35.65 | X | C |
| ATOM | 9527 | CB | VAL | X | 183 | 44.517 | 58.302 | −51.371 | 1.00 | 29.37 | X | C |
| ATOM | 9528 | CG1 | VAL | X | 183 | 44.593 | 58.184 | −49.873 | 1.00 | 23.61 | X | C |
| ATOM | 9529 | CG2 | VAL | X | 183 | 45.840 | 57.923 | −52.016 | 1.00 | 24.98 | X | C |
| ATOM | 9530 | C | VAL | X | 183 | 43.398 | 57.553 | −53.439 | 1.00 | 44.85 | X | C |
| ATOM | 9531 | O | VAL | X | 183 | 43.835 | 56.652 | −54.154 | 1.00 | 50.26 | X | O |
| ATOM | 9532 | N | THR | X | 184 | 42.963 | 58.705 | −53.933 | 1.00 | 49.98 | X | N |
| ATOM | 9533 | CA | THR | X | 184 | 42.953 | 58.927 | −55.373 | 1.00 | 53.09 | X | C |
| ATOM | 9534 | CB | THR | X | 184 | 42.237 | 60.232 | −55.721 | 1.00 | 56.86 | X | C |
| ATOM | 9535 | OG1 | THR | X | 184 | 42.963 | 61.336 | −55.167 | 1.00 | 63.35 | X | O |
| ATOM | 9536 | CG2 | THR | X | 184 | 40.814 | 60.226 | −55.146 | 1.00 | 59.24 | X | C |
| ATOM | 9537 | C | THR | X | 184 | 44.388 | 58.954 | −55.903 | 1.00 | 50.26 | X | C |
| ATOM | 9538 | O | THR | X | 184 | 45.301 | 59.398 | −55.211 | 1.00 | 51.31 | X | O |
| ATOM | 9539 | N | SER | X | 185 | 44.581 | 58.450 | −57.119 | 1.00 | 54.46 | X | N |
| ATOM | 9540 | CA | SER | X | 185 | 45.905 | 58.389 | −57.755 | 1.00 | 54.86 | X | C |
| ATOM | 9541 | CB | SER | X | 185 | 45.844 | 57.586 | −59.053 | 1.00 | 57.17 | X | C |
| ATOM | 9542 | OG | SER | X | 185 | 45.407 | 56.262 | −58.807 | 1.00 | 60.74 | X | O |
| ATOM | 9543 | C | SER | X | 185 | 46.544 | 59.742 | −58.032 | 1.00 | 55.06 | X | C |
| ATOM | 9544 | O | SER | X | 185 | 47.767 | 59.848 | −58.145 | 1.00 | 54.82 | X | O |
| ATOM | 9545 | N | SER | X | 186 | 45.718 | 60.766 | −58.199 | 1.00 | 57.41 | X | N |
| ATOM | 9546 | CA | SER | X | 186 | 46.225 | 62.113 | −58.431 | 1.00 | 53.58 | X | C |
| ATOM | 9547 | CB | SER | X | 186 | 45.107 | 63.025 | −58.953 | 1.00 | 52.85 | X | C |
| ATOM | 9548 | OG | SER | X | 186 | 43.883 | 62.328 | −59.128 | 1.00 | 49.58 | X | O |
| ATOM | 9549 | C | SER | X | 186 | 46.796 | 62.668 | −57.115 | 1.00 | 52.68 | X | C |
| ATOM | 9550 | O | SER | X | 186 | 47.590 | 63.603 | −57.122 | 1.00 | 56.07 | X | O |
| ATOM | 9551 | N | THR | X | 187 | 46.403 | 62.049 | −56.001 | 1.00 | 53.73 | X | N |
| ATOM | 9552 | CA | THR | X | 187 | 46.825 | 62.434 | −54.653 | 1.00 | 51.41 | X | C |
| ATOM | 9553 | CB | THR | X | 187 | 45.782 | 61.961 | −53.607 | 1.00 | 47.98 | X | C |
| ATOM | 9554 | OG1 | THR | X | 187 | 44.563 | 62.680 | −53.816 | 1.00 | 52.63 | X | O |
| ATOM | 9555 | CG2 | THR | X | 187 | 46.260 | 62.196 | −52.175 | 1.00 | 50.32 | X | C |
| ATOM | 9556 | C | THR | X | 187 | 48.212 | 61.936 | −54.250 | 1.00 | 48.36 | X | C |
| ATOM | 9557 | O | THR | X | 187 | 48.906 | 62.607 | −53.510 | 1.00 | 53.35 | X | O |
| ATOM | 9558 | N | TRP | X | 188 | 48.650 | 60.802 | −54.781 | 1.00 | 44.46 | X | N |
| ATOM | 9559 | CA | TRP | X | 188 | 49.950 | 60.259 | −54.401 | 1.00 | 38.84 | X | C |
| ATOM | 9560 | CB | TRP | X | 188 | 49.719 | 59.098 | −53.427 | 1.00 | 35.66 | X | C |
| ATOM | 9561 | CG | TRP | X | 188 | 50.964 | 58.448 | −52.890 | 1.00 | 35.48 | X | C |
| ATOM | 9562 | CD2 | TRP | X | 188 | 51.611 | 57.283 | −53.414 | 1.00 | 35.15 | X | C |
| ATOM | 9563 | CE2 | TRP | X | 188 | 52.703 | 56.996 | −52.566 | 1.00 | 31.52 | X | C |
| ATOM | 9564 | CE3 | TRP | X | 188 | 51.373 | 56.454 | −54.521 | 1.00 | 34.92 | X | C |
| ATOM | 9565 | CD1 | TRP | X | 188 | 51.674 | 58.814 | −51.776 | 1.00 | 26.40 | X | C |
| ATOM | 9566 | NE1 | TRP | X | 188 | 52.712 | 57.941 | −51.574 | 1.00 | 28.05 | X | N |
| ATOM | 9567 | CZ2 | TRP | X | 188 | 53.559 | 55.909 | −52.789 | 1.00 | 34.20 | X | C |
| ATOM | 9568 | CZ3 | TRP | X | 188 | 52.226 | 55.372 | −54.742 | 1.00 | 36.29 | X | C |
| ATOM | 9569 | CH2 | TRP | X | 188 | 53.305 | 55.112 | −53.879 | 1.00 | 35.77 | X | C |
| ATOM | 9570 | C | TRP | X | 188 | 50.764 | 59.804 | −55.614 | 1.00 | 40.48 | X | C |
| ATOM | 9571 | O | TRP | X | 188 | 50.213 | 59.246 | −56.556 | 1.00 | 43.31 | X | O |
| ATOM | 9572 | N | PRO | X | 189 | 52.088 | 60.032 | −55.608 | 1.00 | 37.89 | X | N |
| ATOM | 9573 | CD | PRO | X | 189 | 52.929 | 59.450 | −56.670 | 1.00 | 40.74 | X | C |
| ATOM | 9574 | CA | PRO | X | 189 | 52.929 | 60.663 | −54.590 | 1.00 | 39.18 | X | C |
| ATOM | 9575 | CB | PRO | X | 189 | 54.276 | 60.007 | −54.831 | 1.00 | 39.94 | X | C |
| ATOM | 9576 | CG | PRO | X | 189 | 54.323 | 59.979 | −56.318 | 1.00 | 41.58 | X | C |
| ATOM | 9577 | C | PRO | X | 189 | 53.071 | 62.175 | −54.697 | 1.00 | 40.78 | X | C |
| ATOM | 9578 | O | PRO | X | 189 | 54.043 | 62.730 | −54.188 | 1.00 | 46.43 | X | O |
| ATOM | 9579 | N | SER | X | 190 | 52.152 | 62.841 | −55.389 | 1.00 | 41.15 | X | N |
| ATOM | 9580 | CA | SER | X | 190 | 52.227 | 64.297 | −55.506 | 1.00 | 41.87 | X | C |
| ATOM | 9581 | CB | SER | X | 190 | 51.218 | 64.805 | −56.530 | 1.00 | 40.99 | X | C |
| ATOM | 9582 | OG | SER | X | 190 | 51.241 | 66.223 | −56.591 | 1.00 | 49.19 | X | O |
| ATOM | 9583 | C | SER | X | 190 | 51.940 | 64.929 | −54.143 | 1.00 | 42.44 | X | C |
| ATOM | 9584 | O | SER | X | 190 | 52.527 | 65.942 | −53.772 | 1.00 | 45.68 | X | O |
| ATOM | 9585 | N | GLN | X | 191 | 51.016 | 64.312 | −53.415 | 1.00 | 47.10 | X | N |
| ATOM | 9586 | CA | GLN | X | 191 | 50.605 | 64.744 | −52.079 | 1.00 | 45.36 | X | C |
| ATOM | 9587 | CB | GLN | X | 191 | 49.085 | 64.902 | −52.042 | 1.00 | 43.81 | X | C |
| ATOM | 9588 | CG | GLN | X | 191 | 48.596 | 66.056 | −51.253 | 1.00 | 46.80 | X | C |
| ATOM | 9589 | CD | GLN | X | 191 | 49.121 | 67.349 | −51.780 | 1.00 | 45.68 | X | C |
| ATOM | 9590 | OE1 | GLN | X | 191 | 49.201 | 67.545 | −52.991 | 1.00 | 44.45 | X | O |
| ATOM | 9591 | NE2 | GLN | X | 191 | 49.508 | 68.242 | −50.877 | 1.00 | 46.45 | X | N |
| ATOM | 9592 | C | GLN | X | 191 | 51.034 | 63.613 | −51.140 | 1.00 | 45.54 | X | C |
| ATOM | 9593 | O | GLN | X | 191 | 50.773 | 62.441 | −51.421 | 1.00 | 46.03 | X | O |
| ATOM | 9594 | N | SER | X | 192 | 51.684 | 63.962 | −50.032 | 1.00 | 39.77 | X | N |
| ATOM | 9595 | CA | SER | X | 192 | 52.184 | 62.973 | −49.079 | 1.00 | 34.01 | X | C |
| ATOM | 9596 | CB | SER | X | 192 | 53.204 | 63.631 | −48.141 | 1.00 | 35.39 | X | C |
| ATOM | 9597 | OG | SER | X | 192 | 52.564 | 64.551 | −47.263 | 1.00 | 38.29 | X | O |
| ATOM | 9598 | C | SER | X | 192 | 51.139 | 62.207 | −48.255 | 1.00 | 34.26 | X | C |
| ATOM | 9599 | O | SER | X | 192 | 50.092 | 62.751 | −47.882 | 1.00 | 34.81 | X | O |
| ATOM | 9600 | N | VAL | X | 193 | 51.444 | 60.936 | −47.986 | 1.00 | 27.40 | X | N |
| ATOM | 9601 | CA | VAL | X | 193 | 50.601 | 60.050 | −47.179 | 1.00 | 27.16 | X | C |
| ATOM | 9602 | CB | VAL | X | 193 | 50.001 | 58.864 | −48.003 | 1.00 | 28.74 | X | C |
| ATOM | 9603 | CG1 | VAL | X | 193 | 49.167 | 57.969 | −47.095 | 1.00 | 23.60 | X | C |
| ATOM | 9604 | CG2 | VAL | X | 193 | 49.133 | 59.393 | −49.141 | 1.00 | 28.19 | X | C |
| ATOM | 9605 | C | VAL | X | 193 | 51.526 | 59.493 | −46.109 | 1.00 | 22.85 | X | C |
| ATOM | 9606 | O | VAL | X | 193 | 52.553 | 58.894 | −46.426 | 1.00 | 27.85 | X | O |
| ATOM | 9607 | N | THR | X | 194 | 51.119 | 59.613 | −44.853 | 1.00 | 26.51 | X | N |
| ATOM | 9608 | CA | THR | X | 194 | 51.952 | 59.188 | −43.731 | 1.00 | 23.86 | X | C |
| ATOM | 9609 | CB | THR | X | 194 | 52.562 | 60.447 | −43.058 | 1.00 | 23.49 | X | C |
| ATOM | 9610 | OG1 | THR | X | 194 | 53.220 | 61.224 | −44.064 | 1.00 | 15.27 | X | O |
| ATOM | 9611 | CG2 | THR | X | 194 | 53.555 | 60.081 | −41.948 | 1.00 | 15.74 | X | C |
| ATOM | 9612 | C | THR | X | 194 | 51.170 | 58.420 | −42.686 | 1.00 | 26.58 | X | C |
| ATOM | 9613 | O | THR | X | 194 | 50.112 | 58.872 | −42.260 | 1.00 | 27.83 | X | O |
| ATOM | 9614 | N | CYS | X | 195 | 51.709 | 57.275 | −42.266 | 0.50 | 26.06 | X | N |
| ATOM | 9615 | CA | CYS | X | 195 | 51.088 | 56.393 | −41.269 | 0.50 | 29.25 | X | C |
| ATOM | 9616 | C | CYS | X | 195 | 51.590 | 56.719 | −39.851 | 0.50 | 28.39 | X | C |
| ATOM | 9617 | O | CYS | X | 195 | 52.796 | 56.731 | −39.604 | 0.50 | 30.26 | X | O |
| ATOM | 9618 | CB | CYS | X | 195 | 51.361 | 54.929 | −41.684 | 0.50 | 27.47 | X | C |
| ATOM | 9619 | SG | CYS | X | 195 | 51.841 | 53.681 | −40.444 | 0.50 | 36.46 | X | S |
| ATOM | 9620 | N | ASN | X | 196 | 50.668 | 57.003 | −38.928 | 1.00 | 26.08 | X | N |
| ATOM | 9621 | CA | ASN | X | 196 | 51.050 | 57.360 | −37.555 | 1.00 | 27.12 | X | C |
| ATOM | 9622 | CB | ASN | X | 196 | 50.263 | 58.586 | −37.056 | 1.00 | 23.71 | X | C |
| ATOM | 9623 | CG | ASN | X | 196 | 50.195 | 59.711 | −38.083 | 1.00 | 30.73 | X | C |
| ATOM | 9624 | OD1 | ASN | X | 196 | 49.138 | 59.961 | −38.653 | 1.00 | 33.78 | X | O |
| ATOM | 9625 | ND2 | ASN | X | 196 | 51.318 | 60.392 | −38.320 | 1.00 | 27.31 | X | N |
| ATOM | 9626 | C | ASN | X | 196 | 50.781 | 56.199 | −36.613 | 1.00 | 27.65 | X | C |
| ATOM | 9627 | O | ASN | X | 196 | 49.641 | 55.970 | −36.222 | 1.00 | 31.25 | X | O |
| ATOM | 9628 | N | VAL | X | 197 | 51.837 | 55.482 | −36.240 | 1.00 | 27.91 | X | N |
| ATOM | 9629 | CA | VAL | X | 197 | 51.719 | 54.339 | −35.346 | 1.00 | 23.71 | X | C |
| ATOM | 9630 | CB | VAL | X | 197 | 52.656 | 53.194 | −35.783 | 1.00 | 23.18 | X | C |
| ATOM | 9631 | CG1 | VAL | X | 197 | 52.362 | 51.930 | −34.982 | 1.00 | 15.53 | X | C |
| ATOM | 9632 | CG2 | VAL | X | 197 | 52.510 | 52.941 | −37.274 | 1.00 | 22.35 | X | C |
| ATOM | 9633 | C | VAL | X | 197 | 52.071 | 54.714 | −33.913 | 1.00 | 30.12 | X | C |
| ATOM | 9634 | O | VAL | X | 197 | 53.031 | 55.447 | −33.659 | 1.00 | 32.52 | X | O |
| ATOM | 9635 | N | ALA | X | 198 | 51.296 | 54.197 | −32.970 | 1.00 | 30.37 | X | N |
| ATOM | 9636 | CA | ALA | X | 198 | 51.547 | 54.470 | −31.568 | 1.00 | 26.25 | X | C |
| ATOM | 9637 | CB | ALA | X | 198 | 50.529 | 55.469 | −31.013 | 1.00 | 26.33 | X | C |
| ATOM | 9638 | C | ALA | X | 198 | 51.476 | 53.175 | −30.820 | 1.00 | 20.01 | X | C |
| ATOM | 9639 | O | ALA | X | 198 | 50.614 | 52.355 | −31.085 | 1.00 | 23.58 | X | O |
| ATOM | 9640 | N | HIS | X | 199 | 52.443 | 52.955 | −29.940 | 1.00 | 26.69 | X | N |
| ATOM | 9641 | CA | HIS | X | 199 | 52.475 | 51.747 | −29.133 | 1.00 | 28.14 | X | C |
| ATOM | 9642 | CB | HIS | X | 199 | 53.694 | 50.903 | −29.480 | 1.00 | 26.56 | X | C |
| ATOM | 9643 | CG | HIS | X | 199 | 53.757 | 49.606 | −28.739 | 1.00 | 25.70 | X | C |
| ATOM | 9644 | CD2 | HIS | X | 199 | 52.909 | 48.553 | −28.707 | 1.00 | 31.10 | X | C |
| ATOM | 9645 | ND1 | HIS | X | 199 | 54.818 | 49.266 | −27.929 | 1.00 | 31.09 | X | N |
| ATOM | 9646 | CE1 | HIS | X | 199 | 54.624 | 48.059 | −27.433 | 1.00 | 32.20 | X | C |
| ATOM | 9647 | NE2 | HIS | X | 199 | 53.472 | 47.603 | −27.890 | 1.00 | 33.52 | X | N |
| ATOM | 9648 | C | HIS | X | 199 | 52.504 | 52.172 | −27.674 | 1.00 | 27.57 | X | C |
| ATOM | 9649 | O | HIS | X | 199 | 53.560 | 52.246 | −27.065 | 1.00 | 37.89 | X | O |
| ATOM | 9650 | N | PRO | X | 200 | 51.326 | 52.434 | −27.090 | 1.00 | 31.76 | X | N |
| ATOM | 9651 | CD | PRO | X | 200 | 50.018 | 52.247 | −27.748 | 1.00 | 27.94 | X | C |
| ATOM | 9652 | CA | PRO | X | 200 | 51.143 | 52.863 | −25.701 | 1.00 | 30.23 | X | C |
| ATOM | 9653 | CB | PRO | X | 200 | 49.654 | 52.648 | −25.488 | 1.00 | 34.52 | X | C |
| ATOM | 9654 | CG | PRO | X | 200 | 49.087 | 53.003 | −26.838 | 1.00 | 27.49 | X | C |
| ATOM | 9655 | C | PRO | X | 200 | 51.978 | 52.136 | −24.656 | 1.00 | 34.79 | X | C |
| ATOM | 9656 | O | PRO | X | 200 | 52.571 | 52.778 | −23.779 | 1.00 | 34.65 | X | O |
| ATOM | 9657 | N | ALA | X | 201 | 52.081 | 50.812 | −24.774 | 1.00 | 33.28 | X | N |
| ATOM | 9658 | CA | ALA | X | 201 | 52.854 | 50.043 | −23.799 | 1.00 | 35.46 | X | C |
| ATOM | 9659 | CB | ALA | X | 201 | 52.732 | 48.560 | −24.053 | 1.00 | 32.99 | X | C |
| ATOM | 9660 | C | ALA | X | 201 | 54.318 | 50.427 | −23.725 | 1.00 | 33.63 | X | C |
| ATOM | 9661 | O | ALA | X | 201 | 54.933 | 50.308 | −22.668 | 1.00 | 38.54 | X | O |
| ATOM | 9662 | N | SER | X | 202 | 54.893 | 50.849 | −24.845 | 1.00 | 30.20 | X | N |
| ATOM | 9663 | CA | SER | X | 202 | 56.310 | 51.219 | −24.857 | 1.00 | 28.51 | X | C |
| ATOM | 9664 | CB | SER | X | 202 | 57.066 | 50.451 | −25.954 | 1.00 | 28.60 | X | C |
| ATOM | 9665 | OG | SER | X | 202 | 56.680 | 50.879 | −27.251 | 1.00 | 30.40 | X | O |
| ATOM | 9666 | C | SER | X | 202 | 56.475 | 52.713 | −25.040 | 1.00 | 23.68 | X | C |
| ATOM | 9667 | O | SER | X | 202 | 57.570 | 53.196 | −25.277 | 1.00 | 27.75 | X | O |
| ATOM | 9668 | N | SER | X | 203 | 55.361 | 53.430 | −24.944 | 1.00 | 29.78 | X | N |
| ATOM | 9669 | CA | SER | X | 203 | 55.332 | 54.881 | −25.097 | 1.00 | 34.10 | X | C |
| ATOM | 9670 | CB | SER | X | 203 | 55.765 | 55.570 | −23.790 | 1.00 | 27.22 | X | C |
| ATOM | 9671 | OG | SER | X | 203 | 54.886 | 55.236 | −22.729 | 0.00 | 30.76 | X | O |
| ATOM | 9672 | C | SER | X | 203 | 56.140 | 55.353 | −26.323 | 1.00 | 33.71 | X | C |
| ATOM | 9673 | O | SER | X | 203 | 56.858 | 56.355 | −26.287 | 1.00 | 33.91 | X | O |
| ATOM | 9674 | N | THR | X | 204 | 55.913 | 54.644 | −27.430 | 1.00 | 32.65 | X | N |
| ATOM | 9675 | CA | THR | X | 204 | 56.547 | 54.885 | −28.720 | 1.00 | 27.93 | X | C |
| ATOM | 9676 | CB | THR | X | 204 | 57.177 | 53.567 | −29.227 | 1.00 | 29.10 | X | C |
| ATOM | 9677 | OG1 | THR | X | 204 | 58.259 | 53.200 | −28.369 | 1.00 | 29.14 | X | O |
| ATOM | 9678 | CG2 | THR | X | 204 | 57.674 | 53.686 | −30.665 | 1.00 | 26.15 | X | C |
| ATOM | 9679 | C | THR | X | 204 | 55.545 | 55.391 | −29.776 | 1.00 | 28.05 | X | C |
| ATOM | 9680 | O | THR | X | 204 | 54.388 | 54.997 | −29.789 | 1.00 | 33.86 | X | O |
| ATOM | 9681 | N | LYS | X | 205 | 56.008 | 56.279 | −30.646 | 1.00 | 25.65 | X | N |
| ATOM | 9682 | CA | LYS | X | 205 | 55.211 | 56.826 | −31.736 | 1.00 | 25.98 | X | C |
| ATOM | 9683 | CB | LYS | X | 205 | 54.779 | 58.262 | −31.439 | 1.00 | 28.73 | X | C |
| ATOM | 9684 | CG | LYS | X | 205 | 53.594 | 58.408 | −30.488 | 1.00 | 28.25 | X | C |
| ATOM | 9685 | CD | LYS | X | 205 | 53.525 | 59.820 | −29.943 | 1.00 | 34.25 | X | C |
| ATOM | 9686 | CE | LYS | X | 205 | 52.124 | 60.397 | −30.012 | 1.00 | 42.97 | X | C |
| ATOM | 9687 | NZ | LYS | X | 205 | 52.145 | 61.818 | −29.546 | 1.00 | 50.89 | X | N |
| ATOM | 9688 | C | LYS | X | 205 | 56.131 | 56.806 | −32.958 | 1.00 | 29.61 | X | C |
| ATOM | 9689 | O | LYS | X | 205 | 57.325 | 57.071 | −32.846 | 1.00 | 28.77 | X | O |
| ATOM | 9690 | N | VAL | X | 206 | 55.597 | 56.411 | −34.107 | 1.00 | 27.71 | X | N |
| ATOM | 9691 | CA | VAL | X | 206 | 56.398 | 56.357 | −35.319 | 1.00 | 24.39 | X | C |
| ATOM | 9692 | CB | VAL | X | 206 | 56.838 | 54.904 | −35.682 | 1.00 | 27.49 | X | C |
| ATOM | 9693 | CG1 | VAL | X | 206 | 57.715 | 54.911 | −36.940 | 1.00 | 19.09 | X | C |
| ATOM | 9694 | CG2 | VAL | X | 206 | 57.578 | 54.236 | −34.527 | 1.00 | 22.61 | X | C |
| ATOM | 9695 | C | VAL | X | 206 | 55.568 | 56.873 | −36.470 | 1.00 | 28.55 | X | C |
| ATOM | 9696 | O | VAL | X | 206 | 54.385 | 56.536 | −36.606 | 1.00 | 27.53 | X | O |
| ATOM | 9697 | N | ASP | X | 207 | 56.177 | 57.736 | −37.269 | 1.00 | 24.47 | X | N |
| ATOM | 9698 | CA | ASP | X | 207 | 55.517 | 58.269 | −38.441 | 1.00 | 29.02 | X | C |
| ATOM | 9699 | CB | ASP | X | 207 | 55.561 | 59.791 | −38.436 | 1.00 | 29.91 | X | C |
| ATOM | 9700 | CG | ASP | X | 207 | 54.598 | 60.402 | −37.438 | 1.00 | 37.27 | X | C |
| ATOM | 9701 | OD1 | ASP | X | 207 | 53.733 | 59.678 | −36.902 | 1.00 | 42.89 | X | O |
| ATOM | 9702 | OD2 | ASP | X | 207 | 54.692 | 61.622 | −37.200 | 1.00 | 46.08 | X | O |
| ATOM | 9703 | C | ASP | X | 207 | 56.247 | 57.718 | −39.656 | 1.00 | 31.04 | X | C |
| ATOM | 9704 | O | ASP | X | 207 | 57.462 | 57.860 | −39.767 | 1.00 | 40.84 | X | O |
| ATOM | 9705 | N | LYS | X | 208 | 55.533 | 57.010 | −40.522 | 1.00 | 31.70 | X | N |
| ATOM | 9706 | CA | LYS | X | 208 | 56.166 | 56.461 | −41.711 | 1.00 | 28.44 | X | C |
| ATOM | 9707 | CB | LYS | X | 208 | 56.082 | 54.942 | −41.731 | 1.00 | 29.78 | X | C |
| ATOM | 9708 | CG | LYS | X | 208 | 56.982 | 54.302 | −42.796 | 1.00 | 35.15 | X | C |
| ATOM | 9709 | CD | LYS | X | 208 | 58.478 | 54.432 | −42.456 | 1.00 | 32.64 | X | C |
| ATOM | 9710 | CE | LYS | X | 208 | 59.312 | 54.273 | −43.725 | 1.00 | 38.63 | X | C |
| ATOM | 9711 | NZ | LYS | X | 208 | 60.797 | 54.297 | −43.538 | 1.00 | 39.81 | X | N |
| ATOM | 9712 | C | LYS | X | 208 | 55.568 | 57.044 | −42.980 | 1.00 | 28.71 | X | C |
| ATOM | 9713 | O | LYS | X | 208 | 54.377 | 56.875 | −43.270 | 1.00 | 29.37 | X | O |
| ATOM | 9714 | N | LYS | X | 209 | 56.394 | 57.797 | −43.693 | 1.00 | 26.38 | X | N |
| ATOM | 9715 | CA | LYS | X | 209 | 55.990 | 58.421 | −44.939 | 1.00 | 28.05 | X | C |
| ATOM | 9716 | CB | LYS | X | 209 | 56.888 | 59.622 | −45.222 | 1.00 | 27.93 | X | C |
| ATOM | 9717 | CG | LYS | X | 209 | 56.672 | 60.340 | −46.551 | 1.00 | 35.71 | X | C |
| ATOM | 9718 | CD | LYS | X | 209 | 57.658 | 61.513 | −46.661 | 1.00 | 40.77 | X | C |
| ATOM | 9719 | CE | LYS | X | 209 | 57.855 | 62.023 | −48.093 | 1.00 | 46.39 | X | C |
| ATOM | 9720 | NZ | LYS | X | 209 | 56.630 | 62.612 | −48.710 | 1.00 | 54.22 | X | N |
| ATOM | 9721 | C | LYS | X | 209 | 56.115 | 57.355 | −46.019 | 1.00 | 27.60 | X | C |
| ATOM | 9722 | O | LYS | X | 209 | 57.138 | 56.680 | −46.127 | 1.00 | 28.51 | X | O |
| ATOM | 9723 | N | ILE | X | 210 | 55.036 | 57.152 | −46.763 | 1.00 | 30.55 | X | N |
| ATOM | 9724 | CA | ILE | X | 210 | 55.025 | 56.146 | −47.826 | 1.00 | 30.55 | X | C |
| ATOM | 9725 | CB | ILE | X | 210 | 53.621 | 55.503 | −47.970 | 1.00 | 32.07 | X | C |
| ATOM | 9726 | CG2 | ILE | X | 210 | 53.543 | 54.668 | −49.228 | 1.00 | 31.45 | X | C |
| ATOM | 9727 | CG1 | ILE | X | 210 | 53.317 | 54.630 | −46.759 | 1.00 | 30.39 | X | C |
| ATOM | 9728 | CD1 | ILE | X | 210 | 52.920 | 55.384 | −45.553 | 1.00 | 41.69 | X | C |
| ATOM | 9729 | C | ILE | X | 210 | 55.459 | 56.737 | −49.162 | 1.00 | 25.47 | X | C |
| ATOM | 9730 | O | ILE | X | 210 | 54.777 | 57.596 | −49.710 | 1.00 | 20.90 | X | O |
| ATOM | 9731 | N | VAL | X | 211 | 56.600 | 56.289 | −49.672 | 1.00 | 28.63 | X | N |
| ATOM | 9732 | CA | VAL | X | 211 | 57.093 | 56.794 | −50.942 | 1.00 | 32.50 | X | C |
| ATOM | 9733 | CB | VAL | X | 211 | 58.471 | 57.505 | −50.787 | 1.00 | 38.45 | X | C |
| ATOM | 9734 | CG1 | VAL | X | 211 | 58.377 | 58.627 | −49.745 | 1.00 | 41.44 | X | C |
| ATOM | 9735 | CG2 | VAL | X | 211 | 59.555 | 56.513 | −50.418 | 1.00 | 36.84 | X | C |
| ATOM | 9736 | C | VAL | X | 211 | 57.181 | 55.667 | −51.970 | 1.00 | 37.02 | X | C |
| ATOM | 9737 | O | VAL | X | 211 | 57.283 | 54.494 | −51.599 | 1.00 | 33.71 | X | O |
| ATOM | 9738 | N | PRO | X | 212 | 57.134 | 56.013 | −53.276 | 1.00 | 37.99 | X | N |
| ATOM | 9739 | CD | PRO | X | 212 | 56.911 | 57.386 | −53.760 | 1.00 | 38.64 | X | C |
| ATOM | 9740 | CA | PRO | X | 212 | 57.201 | 55.088 | −54.414 | 1.00 | 40.35 | X | C |
| ATOM | 9741 | CB | PRO | X | 212 | 57.164 | 56.031 | −55.614 | 1.00 | 40.19 | X | C |
| ATOM | 9742 | CG | PRO | X | 212 | 56.326 | 57.136 | −55.132 | 1.00 | 43.84 | X | C |
| ATOM | 9743 | C | PRO | X | 212 | 58.456 | 54.236 | −54.434 | 1.00 | 42.42 | X | C |
| ATOM | 9744 | O | PRO | X | 212 | 59.544 | 54.728 | −54.131 | 1.00 | 51.07 | X | O |
| ATOM | 9745 | N | LYS | X | 213 | 58.303 | 52.965 | −54.807 | 1.00 | 46.34 | X | N |
| ATOM | 9746 | CA | LYS | X | 213 | 59.430 | 52.023 | −54.872 | 1.00 | 49.64 | X | C |
| ATOM | 9747 | CB | LYS | X | 213 | 58.933 | 50.598 | −55.125 | 1.00 | 47.19 | X | C |
| ATOM | 9748 | CG | LYS | X | 213 | 58.241 | 49.936 | −53.948 | 1.00 | 45.89 | X | C |
| ATOM | 9749 | CD | LYS | X | 213 | 58.002 | 48.458 | −54.238 | 1.00 | 41.61 | X | C |
| ATOM | 9750 | CE | LYS | X | 213 | 59.236 | 47.594 | −53.992 | 1.00 | 48.40 | X | C |
| ATOM | 9751 | NZ | LYS | X | 213 | 59.394 | 47.307 | −52.522 | 1.00 | 42.38 | X | N |
| ATOM | 9752 | C | LYS | X | 213 | 60.496 | 52.374 | −55.916 | 1.00 | 46.71 | X | C |
| ATOM | 9753 | O | LYS | X | 213 | 60.209 | 53.028 | −56.922 | 1.00 | 55.68 | X | O |
| ATOM | 9754 | N | GLN | Y | 1 | 14.060 | −10.777 | 53.078 | 1.00 | 63.61 | Y | N |
| ATOM | 9755 | CA | GLN | Y | 1 | 13.387 | −9.766 | 52.264 | 1.00 | 65.22 | Y | C |
| ATOM | 9756 | CB | GLN | Y | 1 | 11.916 | −10.150 | 52.024 | 1.00 | 67.22 | Y | C |
| ATOM | 9757 | CG | GLN | Y | 1 | 11.673 | −11.621 | 51.615 | 1.00 | 75.10 | Y | C |
| ATOM | 9758 | CD | GLN | Y | 1 | 12.220 | −11.989 | 50.227 | 1.00 | 79.81 | Y | C |
| ATOM | 9759 | OE1 | GLN | Y | 1 | 13.410 | −11.810 | 49.935 | 1.00 | 82.43 | Y | O |
| ATOM | 9760 | NE2 | GLN | Y | 1 | 11.349 | −12.538 | 49.378 | 1.00 | 79.43 | Y | N |
| ATOM | 9761 | C | GLN | Y | 1 | 13.463 | −8.370 | 52.901 | 1.00 | 60.91 | Y | C |
| ATOM | 9762 | O | GLN | Y | 1 | 13.068 | −7.380 | 52.275 | 1.00 | 64.55 | Y | O |
| ATOM | 9763 | N | ALA | Y | 2 | 13.969 | −8.304 | 54.134 | 1.00 | 51.46 | Y | N |
| ATOM | 9764 | CA | ALA | Y | 2 | 14.127 | −7.055 | 54.891 | 1.00 | 46.94 | Y | C |
| ATOM | 9765 | CB | ALA | Y | 2 | 14.763 | −5.967 | 54.027 | 1.00 | 41.90 | Y | C |
| ATOM | 9766 | C | ALA | Y | 2 | 12.858 | −6.527 | 55.566 | 1.00 | 40.94 | Y | C |
| ATOM | 9767 | O | ALA | Y | 2 | 11.828 | −6.312 | 54.929 | 1.00 | 37.29 | Y | O |
| ATOM | 9768 | N | VAL | Y | 3 | 12.961 | −6.294 | 56.868 | 1.00 | 40.38 | Y | N |
| ATOM | 9769 | CA | VAL | Y | 3 | 11.844 | −5.799 | 57.665 | 1.00 | 39.69 | Y | C |
| ATOM | 9770 | CB | VAL | Y | 3 | 11.516 | −6.796 | 58.831 | 1.00 | 38.36 | Y | C |
| ATOM | 9771 | CG1 | VAL | Y | 3 | 10.408 | −6.253 | 59.719 | 1.00 | 37.79 | Y | C |
| ATOM | 9772 | CG2 | VAL | Y | 3 | 11.115 | −8.162 | 58.258 | 1.00 | 35.60 | Y | C |
| ATOM | 9773 | C | VAL | Y | 3 | 12.144 | −4.400 | 58.210 | 1.00 | 35.02 | Y | C |
| ATOM | 9774 | O | VAL | Y | 3 | 13.225 | −4.142 | 58.736 | 1.00 | 41.14 | Y | O |
| ATOM | 9775 | N | VAL | Y | 4 | 11.173 | −3.507 | 58.080 | 1.00 | 36.13 | Y | N |
| ATOM | 9776 | CA | VAL | Y | 4 | 11.307 | −2.126 | 58.535 | 1.00 | 36.06 | Y | C |
| ATOM | 9777 | CB | VAL | Y | 4 | 10.892 | −1.149 | 57.404 | 1.00 | 35.20 | Y | C |
| ATOM | 9778 | CG1 | VAL | Y | 4 | 11.171 | 0.271 | 57.799 | 1.00 | 37.38 | Y | C |
| ATOM | 9779 | CG2 | VAL | Y | 4 | 11.624 | −1.498 | 56.112 | 1.00 | 39.88 | Y | C |
| ATOM | 9780 | C | VAL | Y | 4 | 10.408 | −1.940 | 59.759 | 1.00 | 36.04 | Y | C |
| ATOM | 9781 | O | VAL | Y | 4 | 9.190 | −2.074 | 59.666 | 1.00 | 38.83 | Y | O |
| ATOM | 9782 | N | THR | Y | 5 | 11.004 | −1.630 | 60.906 | 1.00 | 35.20 | Y | N |
| ATOM | 9783 | CA | THR | Y | 5 | 10.228 | −1.476 | 62.132 | 1.00 | 32.53 | Y | C |
| ATOM | 9784 | CB | THR | Y | 5 | 10.734 | −2.444 | 63.223 | 1.00 | 34.95 | Y | C |
| ATOM | 9785 | OG1 | THR | Y | 5 | 12.157 | −2.381 | 63.283 | 1.00 | 43.46 | Y | O |
| ATOM | 9786 | CG2 | THR | Y | 5 | 10.339 | −3.886 | 62.897 | 1.00 | 37.57 | Y | C |
| ATOM | 9787 | C | THR | Y | 5 | 10.122 | −0.061 | 62.698 | 1.00 | 33.83 | Y | C |
| ATOM | 9788 | O | THR | Y | 5 | 11.108 | 0.664 | 62.780 | 1.00 | 38.05 | Y | O |
| ATOM | 9789 | N | GLN | Y | 6 | 8.901 | 0.329 | 63.063 | 1.00 | 34.55 | Y | N |
| ATOM | 9790 | CA | GLN | Y | 6 | 8.627 | 1.646 | 63.644 | 1.00 | 33.03 | Y | C |
| ATOM | 9791 | CB | GLN | Y | 6 | 7.666 | 2.468 | 62.751 | 1.00 | 32.65 | Y | C |
| ATOM | 9792 | CG | GLN | Y | 6 | 8.153 | 2.795 | 61.346 | 1.00 | 21.06 | Y | C |
| ATOM | 9793 | CD | GLN | Y | 6 | 7.085 | 3.451 | 60.493 | 1.00 | 25.86 | Y | C |
| ATOM | 9794 | OE1 | GLN | Y | 6 | 6.570 | 2.836 | 59.563 | 1.00 | 23.13 | Y | O |
| ATOM | 9795 | NE2 | GLN | Y | 6 | 6.750 | 4.708 | 60.796 | 1.00 | 21.77 | Y | N |
| ATOM | 9796 | C | GLN | Y | 6 | 7.938 | 1.418 | 64.992 | 1.00 | 36.65 | Y | C |
| ATOM | 9797 | O | GLN | Y | 6 | 7.410 | 0.321 | 65.241 | 1.00 | 35.93 | Y | O |
| ATOM | 9798 | N | GLU | Y | 7 | 7.957 | 2.444 | 65.853 | 1.00 | 37.56 | Y | N |
| ATOM | 9799 | CA | GLU | Y | 7 | 7.289 | 2.395 | 67.167 | 1.00 | 37.10 | Y | C |
| ATOM | 9800 | CB | GLU | Y | 7 | 7.486 | 3.721 | 67.923 | 1.00 | 35.37 | Y | C |
| ATOM | 9801 | CG | GLU | Y | 7 | 8.942 | 4.092 | 68.221 | 1.00 | 39.89 | Y | C |
| ATOM | 9802 | CD | GLU | Y | 7 | 9.564 | 5.062 | 67.214 | 1.00 | 40.13 | Y | C |
| ATOM | 9803 | OE1 | GLU | Y | 7 | 9.023 | 5.253 | 66.100 | 1.00 | 40.69 | Y | O |
| ATOM | 9804 | OE2 | GLU | Y | 7 | 10.604 | 5.657 | 67.560 | 1.00 | 37.88 | Y | O |
| ATOM | 9805 | C | GLU | Y | 7 | 5.786 | 2.211 | 66.878 | 1.00 | 38.63 | Y | C |
| ATOM | 9806 | O | GLU | Y | 7 | 5.232 | 2.905 | 66.019 | 1.00 | 35.70 | Y | O |
| ATOM | 9807 | N | SER | Y | 8 | 5.127 | 1.283 | 67.564 | 1.00 | 35.59 | Y | N |
| ATOM | 9808 | CA | SER | Y | 8 | 3.705 | 1.071 | 67.304 | 1.00 | 36.00 | Y | C |
| ATOM | 9809 | CB | SER | Y | 8 | 3.158 | −0.106 | 68.123 | 1.00 | 37.42 | Y | C |
| ATOM | 9810 | OG | SER | Y | 8 | 3.633 | −0.087 | 69.454 | 1.00 | 41.06 | Y | O |
| ATOM | 9811 | C | SER | Y | 8 | 2.873 | 2.340 | 67.502 | 1.00 | 35.54 | Y | C |
| ATOM | 9812 | O | SER | Y | 8 | 2.161 | 2.776 | 66.587 | 1.00 | 34.38 | Y | O |
| ATOM | 9813 | N | ALA | Y | 9 | 3.015 | 2.972 | 68.662 | 1.00 | 32.74 | Y | N |
| ATOM | 9814 | CA | ALA | Y | 9 | 2.271 | 4.194 | 68.940 | 1.00 | 31.80 | Y | C |
| ATOM | 9815 | CB | ALA | Y | 9 | 0.958 | 3.857 | 69.633 | 1.00 | 24.95 | Y | C |
| ATOM | 9816 | C | ALA | Y | 9 | 3.087 | 5.143 | 69.805 | 1.00 | 32.47 | Y | C |
| ATOM | 9817 | O | ALA | Y | 9 | 3.957 | 4.709 | 70.550 | 1.00 | 34.96 | Y | O |
| ATOM | 9818 | N | LEU | Y | 11 | 2.829 | 6.441 | 69.666 | 1.00 | 33.04 | Y | N |
| ATOM | 9819 | CA | LEU | Y | 11 | 3.506 | 7.455 | 70.472 | 1.00 | 34.88 | Y | C |
| ATOM | 9820 | CB | LEU | Y | 11 | 4.694 | 8.089 | 69.735 | 1.00 | 34.66 | Y | C |
| ATOM | 9821 | CG | LEU | Y | 11 | 5.972 | 7.287 | 69.490 | 1.00 | 38.06 | Y | C |
| ATOM | 9822 | CD1 | LEU | Y | 11 | 6.971 | 8.235 | 68.878 | 1.00 | 34.82 | Y | C |
| ATOM | 9823 | CD2 | LEU | Y | 11 | 6.543 | 6.673 | 70.776 | 1.00 | 36.71 | Y | C |
| ATOM | 9824 | C | LEU | Y | 11 | 2.523 | 8.544 | 70.858 | 1.00 | 33.04 | Y | C |
| ATOM | 9825 | O | LEU | Y | 11 | 1.684 | 8.969 | 70.052 | 1.00 | 30.66 | Y | O |
| ATOM | 9826 | N | THR | Y | 12 | 2.646 | 9.015 | 72.090 | 1.00 | 32.22 | Y | N |
| ATOM | 9827 | CA | THR | Y | 12 | 1.767 | 10.066 | 72.563 | 1.00 | 35.86 | Y | C |
| ATOM | 9828 | CB | THR | Y | 12 | 0.798 | 9.531 | 73.694 | 1.00 | 35.63 | Y | C |
| ATOM | 9829 | OG1 | THR | Y | 12 | 0.091 | 10.623 | 74.290 | 1.00 | 38.91 | Y | O |
| ATOM | 9830 | CG2 | THR | Y | 12 | 1.548 | 8.742 | 74.766 | 1.00 | 39.32 | Y | C |
| ATOM | 9831 | C | THR | Y | 12 | 2.543 | 11.336 | 72.951 | 1.00 | 32.19 | Y | C |
| ATOM | 9832 | O | THR | Y | 12 | 3.583 | 11.275 | 73.602 | 1.00 | 38.02 | Y | O |
| ATOM | 9833 | N | SER | Y | 13 | 2.073 | 12.477 | 72.459 | 1.00 | 32.22 | Y | N |
| ATOM | 9834 | CA | SER | Y | 13 | 2.699 | 13.759 | 72.744 | 1.00 | 31.28 | Y | C |
| ATOM | 9835 | CB | SER | Y | 13 | 3.524 | 14.210 | 71.553 | 1.00 | 35.87 | Y | C |
| ATOM | 9836 | OG | SER | Y | 13 | 2.676 | 14.692 | 70.531 | 1.00 | 44.66 | Y | O |
| ATOM | 9837 | C | SER | Y | 13 | 1.650 | 14.822 | 73.045 | 1.00 | 26.36 | Y | C |
| ATOM | 9838 | O | SER | Y | 13 | 0.462 | 14.561 | 72.965 | 1.00 | 33.61 | Y | O |
| ATOM | 9839 | N | SER | Y | 14 | 2.100 | 16.041 | 73.317 | 1.00 | 22.66 | Y | N |
| ATOM | 9840 | CA | SER | Y | 14 | 1.198 | 17.143 | 73.634 | 1.00 | 22.00 | Y | C |
| ATOM | 9841 | CB | SER | Y | 14 | 1.400 | 17.612 | 75.087 | 1.00 | 25.73 | Y | C |
| ATOM | 9842 | OG | SER | Y | 14 | 1.369 | 16.533 | 76.013 | 1.00 | 31.36 | Y | O |
| ATOM | 9843 | C | SER | Y | 14 | 1.493 | 18.310 | 72.714 | 1.00 | 21.31 | Y | C |
| ATOM | 9844 | O | SER | Y | 14 | 2.571 | 18.397 | 72.137 | 1.00 | 26.95 | Y | O |
| ATOM | 9845 | N | PRO | Y | 15 | 0.545 | 19.242 | 72.578 | 1.00 | 21.30 | Y | N |
| ATOM | 9846 | CD | PRO | Y | 15 | −0.814 | 19.302 | 73.141 | 1.00 | 28.85 | Y | C |
| ATOM | 9847 | CA | PRO | Y | 15 | 0.793 | 20.382 | 71.705 | 1.00 | 26.27 | Y | C |
| ATOM | 9848 | CB | PRO | Y | 15 | −0.468 | 21.237 | 71.879 | 1.00 | 27.12 | Y | C |
| ATOM | 9849 | CG | PRO | Y | 15 | −1.507 | 20.251 | 72.192 | 1.00 | 28.38 | Y | C |
| ATOM | 9850 | C | PRO | Y | 15 | 2.021 | 21.145 | 72.171 | 1.00 | 30.39 | Y | C |
| ATOM | 9851 | O | PRO | Y | 15 | 2.199 | 21.386 | 73.365 | 1.00 | 32.63 | Y | O |
| ATOM | 9852 | N | GLY | Y | 16 | 2.873 | 21.497 | 71.213 | 1.00 | 36.03 | Y | N |
| ATOM | 9853 | CA | GLY | Y | 16 | 4.077 | 22.250 | 71.503 | 1.00 | 32.07 | Y | C |
| ATOM | 9854 | C | GLY | Y | 16 | 5.295 | 21.398 | 71.733 | 1.00 | 27.60 | Y | C |
| ATOM | 9855 | O | GLY | Y | 16 | 6.409 | 21.908 | 71.697 | 1.00 | 32.88 | Y | O |
| ATOM | 9856 | N | GLU | Y | 17 | 5.095 | 20.100 | 71.936 | 1.00 | 27.36 | Y | N |
| ATOM | 9857 | CA | GLU | Y | 17 | 6.208 | 19.192 | 72.179 | 1.00 | 32.17 | Y | C |
| ATOM | 9858 | CB | GLU | Y | 17 | 5.652 | 17.892 | 72.753 | 1.00 | 38.81 | Y | C |
| ATOM | 9859 | CG | GLU | Y | 17 | 6.622 | 17.045 | 73.553 | 1.00 | 42.47 | Y | C |
| ATOM | 9860 | CD | GLU | Y | 17 | 5.904 | 15.962 | 74.350 | 1.00 | 50.83 | Y | C |
| ATOM | 9861 | OE1 | GLU | Y | 17 | 4.804 | 16.234 | 74.888 | 1.00 | 55.42 | Y | O |
| ATOM | 9862 | OE2 | GLU | Y | 17 | 6.434 | 14.833 | 74.438 | 1.00 | 54.16 | Y | O |
| ATOM | 9863 | CG | GLU | Y | 17 | 7.021 | 18.913 | 70.910 | 1.00 | 37.16 | Y | C |
| ATOM | 9864 | O | GLU | Y | 17 | 6.593 | 19.248 | 69.801 | 1.00 | 37.49 | Y | O |
| ATOM | 9865 | N | THR | Y | 18 | 8.226 | 18.375 | 71.094 | 1.00 | 35.58 | Y | N |
| ATOM | 9866 | CA | THR | Y | 18 | 9.098 | 17.992 | 69.988 | 1.00 | 33.13 | Y | C |
| ATOM | 9867 | CB | THR | Y | 18 | 10.542 | 18.548 | 70.154 | 1.00 | 35.65 | Y | C |
| ATOM | 9868 | OG1 | THR | Y | 18 | 10.564 | 19.946 | 69.844 | 1.00 | 36.26 | Y | O |
| ATOM | 9869 | CG2 | THR | Y | 18 | 11.501 | 17.844 | 69.224 | 1.00 | 31.99 | Y | C |
| ATOM | 9870 | C | THR | Y | 18 | 9.112 | 16.463 | 70.035 | 1.00 | 33.44 | Y | C |
| ATOM | 9871 | O | THR | Y | 18 | 9.563 | 15.876 | 71.024 | 1.00 | 36.33 | Y | O |
| ATOM | 9872 | N | VAL | Y | 19 | 8.582 | 15.827 | 68.986 | 1.00 | 27.53 | Y | N |
| ATOM | 9873 | CA | VAL | Y | 19 | 8.500 | 14.358 | 68.915 | 1.00 | 22.66 | Y | C |
| ATOM | 9874 | CB | VAL | Y | 19 | 7.050 | 13.869 | 68.616 | 1.00 | 23.01 | Y | C |
| ATOM | 9875 | CG1 | VAL | Y | 19 | 6.905 | 12.387 | 68.977 | 1.00 | 22.15 | Y | C |
| ATOM | 9876 | CG2 | VAL | Y | 19 | 6.043 | 14.702 | 69.355 | 1.00 | 20.79 | Y | C |
| ATOM | 9877 | C | VAL | Y | 19 | 9.406 | 13.747 | 67.850 | 1.00 | 18.66 | Y | C |
| ATOM | 9878 | O | VAL | Y | 19 | 9.539 | 14.282 | 66.755 | 1.00 | 25.07 | Y | O |
| ATOM | 9879 | N | THR | Y | 20 | 9.916 | 12.557 | 68.134 | 1.00 | 21.42 | Y | N |
| ATOM | 9880 | CA | THR | Y | 20 | 10.806 | 11.887 | 67.228 | 1.00 | 27.44 | Y | C |
| ATOM | 9881 | CB | THR | Y | 20 | 12.252 | 11.918 | 67.779 | 1.00 | 31.05 | Y | C |
| ATOM | 9882 | OG1 | THR | Y | 20 | 12.683 | 13.282 | 67.893 | 1.00 | 39.89 | Y | O |
| ATOM | 9883 | CG2 | THR | Y | 20 | 13.206 | 11.184 | 66.853 | 1.00 | 34.08 | Y | C |
| ATOM | 9884 | C | THR | Y | 20 | 10.385 | 10.462 | 66.926 | 1.00 | 31.96 | Y | C |
| ATOM | 9885 | O | THR | Y | 20 | 10.440 | 9.582 | 67.795 | 1.00 | 33.59 | Y | O |
| ATOM | 9886 | N | LEU | Y | 21 | 9.959 | 10.251 | 65.680 | 1.00 | 30.78 | Y | N |
| ATOM | 9887 | CA | LEU | Y | 21 | 9.548 | 8.937 | 65.206 | 1.00 | 28.84 | Y | C |
| ATOM | 9888 | CB | LEU | Y | 21 | 8.422 | 9.066 | 64.183 | 1.00 | 34.19 | Y | C |
| ATOM | 9889 | CG | LEU | Y | 21 | 7.206 | 9.956 | 64.498 | 1.00 | 37.68 | Y | C |
| ATOM | 9890 | CD1 | LEU | Y | 21 | 6.109 | 9.640 | 63.510 | 1.00 | 37.22 | Y | C |
| ATOM | 9891 | CD2 | LEU | Y | 21 | 6.691 | 9.724 | 65.891 | 1.00 | 38.19 | Y | C |
| ATOM | 9892 | C | LEU | Y | 21 | 10.782 | 8.362 | 64.542 | 1.00 | 29.40 | Y | C |
| ATOM | 9893 | O | LEU | Y | 21 | 11.538 | 9.098 | 63.915 | 1.00 | 33.27 | Y | O |
| ATOM | 9894 | N | THR | Y | 22 | 11.015 | 7.063 | 64.701 | 1.00 | 27.96 | Y | N |
| ATOM | 9895 | CA | THR | Y | 22 | 12.182 | 6.446 | 64.103 | 1.00 | 26.24 | Y | C |
| ATOM | 9896 | CB | THR | Y | 22 | 13.218 | 6.006 | 65.167 | 1.00 | 26.36 | Y | C |
| ATOM | 9897 | OG1 | THR | Y | 22 | 12.691 | 4.931 | 65.945 | 1.00 | 37.37 | Y | O |
| ATOM | 9898 | CG2 | THR | Y | 22 | 13.560 | 7.156 | 66.094 | 1.00 | 26.39 | Y | C |
| ATOM | 9899 | C | THR | Y | 22 | 11.817 | 5.280 | 63.203 | 1.00 | 29.43 | Y | C |
| ATOM | 9900 | O | THR | Y | 22 | 10.743 | 4.692 | 63.332 | 1.00 | 29.35 | Y | O |
| ATOM | 9901 | N | CYS | Y | 23 | 12.753 | 4.922 | 62.329 | 1.00 | 32.76 | Y | N |
| ATOM | 9902 | CA | CYS | Y | 23 | 12.559 | 3.865 | 61.355 | 1.00 | 32.79 | Y | C |
| ATOM | 9903 | C | CYS | Y | 23 | 13.825 | 3.040 | 61.230 | 1.00 | 35.44 | Y | C |
| ATOM | 9904 | O | CYS | Y | 23 | 14.840 | 3.523 | 60.728 | 1.00 | 34.81 | Y | O |
| ATOM | 9905 | CB | CYS | Y | 23 | 12.266 | 4.514 | 60.024 | 1.00 | 39.39 | Y | C |
| ATOM | 9906 | SG | CYS | Y | 23 | 11.965 | 3.367 | 58.668 | 1.00 | 36.68 | Y | S |
| ATOM | 9907 | N | ARG | Y | 24 | 13.729 | 1.773 | 61.604 | 1.00 | 31.43 | Y | N |
| ATOM | 9908 | CA | ARG | Y | 24 | 14.869 | 0.883 | 61.594 | 1.00 | 30.66 | Y | C |
| ATOM | 9909 | CB | ARG | Y | 24 | 15.020 | 0.256 | 62.992 | 1.00 | 35.56 | Y | C |
| ATOM | 9910 | CG | ARG | Y | 24 | 15.925 | −0.962 | 63.080 | 1.00 | 40.27 | Y | C |
| ATOM | 9911 | CD | ARG | Y | 24 | 16.499 | −1.133 | 64.478 | 1.00 | 50.46 | Y | C |
| ATOM | 9912 | NE | ARG | Y | 24 | 17.030 | −2.481 | 64.683 | 1.00 | 62.36 | Y | N |
| ATOM | 9913 | CZ | ARG | Y | 24 | 18.101 | −2.991 | 64.066 | 1.00 | 68.09 | Y | C |
| ATOM | 9914 | NH1 | ARG | Y | 24 | 18.795 | −2.265 | 63.189 | 1.00 | 69.07 | Y | N |
| ATOM | 9915 | NH2 | ARG | Y | 24 | 18.453 | −4.254 | 64.295 | 1.00 | 66.97 | Y | N |
| ATOM | 9916 | C | ARG | Y | 24 | 14.862 | −0.190 | 60.517 | 1.00 | 30.71 | Y | C |
| ATOM | 9917 | O | ARG | Y | 24 | 13.835 | −0.801 | 60.232 | 1.00 | 28.55 | Y | O |
| ATOM | 9918 | N | SER | Y | 25 | 16.036 | −0.416 | 59.939 | 1.00 | 30.05 | Y | N |
| ATOM | 9919 | CA | SER | Y | 25 | 16.219 | −1.425 | 58.919 | 1.00 | 29.76 | Y | C |
| ATOM | 9920 | CB | SER | Y | 25 | 17.158 | −0.910 | 57.840 | 1.00 | 29.42 | Y | C |
| ATOM | 9921 | OG | SER | Y | 25 | 17.303 | −1.871 | 56.812 | 1.00 | 32.11 | Y | O |
| ATOM | 9922 | C | SER | Y | 25 | 16.827 | −2.663 | 59.561 | 1.00 | 33.31 | Y | C |
| ATOM | 9923 | O | SER | Y | 25 | 17.747 | −2.566 | 60.373 | 1.00 | 40.38 | Y | O |
| ATOM | 9924 | N | SER | Y | 26 | 16.331 | −3.827 | 59.168 | 1.00 | 34.87 | Y | N |
| ATOM | 9925 | CA | SER | Y | 26 | 16.824 | −5.094 | 59.692 | 1.00 | 36.41 | Y | C |
| ATOM | 9926 | CB | SER | Y | 26 | 15.786 | −6.190 | 59.430 | 1.00 | 38.67 | Y | C |
| ATOM | 9927 | OG | SER | Y | 26 | 15.694 | −6.484 | 58.042 | 1.00 | 35.31 | Y | O |
| ATOM | 9928 | C | SER | Y | 26 | 18.170 | −5.543 | 59.102 | 1.00 | 37.72 | Y | C |
| ATOM | 9929 | O | SER | Y | 26 | 18.860 | −6.364 | 59.707 | 1.00 | 37.77 | Y | O |
| ATOM | 9930 | N | THR | Y | 27 | 18.515 | −5.036 | 57.915 | 1.00 | 35.30 | Y | N |
| ATOM | 9931 | CA | THR | Y | 27 | 19.751 | −5.418 | 57.232 | 1.00 | 35.29 | Y | C |
| ATOM | 9932 | CB | THR | Y | 27 | 19.673 | −5.155 | 55.713 | 1.00 | 33.61 | Y | C |
| ATOM | 9933 | OG1 | THR | Y | 27 | 19.644 | −3.744 | 55.465 | 1.00 | 40.01 | Y | O |
| ATOM | 9934 | CG2 | THR | Y | 27 | 18.444 | −5.793 | 55.118 | 1.00 | 35.61 | Y | C |
| ATOM | 9935 | C | THR | Y | 27 | 21.005 | −4.732 | 57.770 | 1.00 | 35.93 | Y | C |
| ATOM | 9936 | O | THR | Y | 27 | 22.109 | −5.264 | 57.648 | 1.00 | 37.86 | Y | O |
| ATOM | 9937 | N | GLY | Y | 27A | 20.828 | −3.545 | 58.338 | 1.00 | 32.97 | Y | N |
| ATOM | 9938 | CA | GLY | Y | 27A | 21.943 | −2.801 | 58.878 | 1.00 | 32.31 | Y | C |
| ATOM | 9939 | C | GLY | Y | 27A | 21.492 | −1.376 | 59.071 | 1.00 | 37.67 | Y | C |
| ATOM | 9940 | O | GLY | Y | 27A | 20.288 | −1.113 | 59.152 | 1.00 | 37.98 | Y | O |
| ATOM | 9941 | N | ALA | Y | 27B | 22.446 | −0.450 | 59.107 | 1.00 | 38.79 | Y | N |
| ATOM | 9942 | CA | ALA | Y | 27B | 22.126 | 0.964 | 59.293 | 1.00 | 37.65 | Y | C |
| ATOM | 9943 | CB | ALA | Y | 27B | 23.396 | 1.789 | 59.550 | 1.00 | 29.47 | Y | C |
| ATOM | 9944 | C | ALA | Y | 27B | 21.381 | 1.534 | 58.100 | 1.00 | 37.75 | Y | C |
| ATOM | 9945 | O | ALA | Y | 27B | 21.655 | 1.186 | 56.946 | 1.00 | 36.01 | Y | O |
| ATOM | 9946 | N | VAL | Y | 27C | 20.421 | 2.398 | 58.393 | 1.00 | 37.73 | Y | N |
| ATOM | 9947 | CA | VAL | Y | 27C | 19.664 | 3.045 | 57.349 | 1.00 | 38.44 | Y | C |
| ATOM | 9948 | CB | VAL | Y | 27C | 18.397 | 3.749 | 57.896 | 1.00 | 37.61 | Y | C |
| ATOM | 9949 | CG1 | VAL | Y | 27C | 17.662 | 4.452 | 56.768 | 1.00 | 38.08 | Y | C |
| ATOM | 9950 | CG2 | VAL | Y | 27C | 17.479 | 2.729 | 58.539 | 1.00 | 35.10 | Y | C |
| ATOM | 9951 | C | VAL | Y | 27C | 20.621 | 4.042 | 56.716 | 1.00 | 36.87 | Y | C |
| ATOM | 9952 | O | VAL | Y | 27C | 21.229 | 4.876 | 57.392 | 1.00 | 37.51 | Y | O |
| ATOM | 9953 | N | THR | Y | 28 | 20.751 | 3.932 | 55.406 | 1.00 | 37.62 | Y | N |
| ATOM | 9954 | CA | THR | Y | 28 | 21.643 | 4.779 | 54.662 | 1.00 | 37.70 | Y | C |
| ATOM | 9955 | CB | THR | Y | 28 | 22.648 | 3.896 | 53.912 | 1.00 | 41.63 | Y | C |
| ATOM | 9956 | OG1 | THR | Y | 28 | 23.580 | 4.722 | 53.211 | 1.00 | 56.22 | Y | O |
| ATOM | 9957 | CG2 | THR | Y | 28 | 21.927 | 2.981 | 52.920 | 1.00 | 41.27 | Y | C |
| ATOM | 9958 | C | THR | Y | 28 | 20.911 | 5.711 | 53.696 | 1.00 | 35.89 | Y | C |
| ATOM | 9959 | O | THR | Y | 28 | 19.712 | 5.572 | 53.442 | 1.00 | 33.41 | Y | O |
| ATOM | 9960 | N | THR | Y | 29 | 21.652 | 6.673 | 53.170 | 1.00 | 31.64 | Y | N |
| ATOM | 9961 | CA | THR | Y | 29 | 21.129 | 7.639 | 52.232 | 1.00 | 28.97 | Y | C |
| ATOM | 9962 | CB | THR | Y | 29 | 22.232 | 8.674 | 51.959 | 1.00 | 27.78 | Y | C |
| ATOM | 9963 | OG1 | THR | Y | 29 | 21.663 | 9.866 | 51.426 | 1.00 | 37.73 | Y | O |
| ATOM | 9964 | CG2 | THR | Y | 29 | 23.255 | 8.126 | 51.022 | 1.00 | 26.05 | Y | C |
| ATOM | 9965 | C | THR | Y | 29 | 20.632 | 6.927 | 50.945 | 1.00 | 32.33 | Y | C |
| ATOM | 9966 | O | THR | Y | 29 | 19.786 | 7.458 | 50.217 | 1.00 | 28.15 | Y | O |
| ATOM | 9967 | N | SER | Y | 30 | 21.134 | 5.709 | 50.706 | 1.00 | 27.72 | Y | N |
| ATOM | 9968 | CA | SER | Y | 30 | 20.744 | 4.907 | 49.549 | 1.00 | 30.20 | Y | C |
| ATOM | 9969 | CB | SER | Y | 30 | 21.741 | 3.777 | 49.299 | 1.00 | 30.77 | Y | C |
| ATOM | 9970 | OG | SER | Y | 30 | 22.970 | 4.283 | 48.822 | 1.00 | 44.61 | Y | O |
| ATOM | 9971 | C | SER | Y | 30 | 19.358 | 4.298 | 49.735 | 1.00 | 30.11 | Y | C |
| ATOM | 9972 | O | SER | Y | 30 | 18.751 | 3.808 | 48.786 | 1.00 | 32.06 | Y | O |
| ATOM | 9973 | N | ASN | Y | 31 | 18.875 | 4.314 | 50.967 | 1.00 | 26.54 | Y | N |
| ATOM | 9974 | CA | ASN | Y | 31 | 17.576 | 3.763 | 51.263 | 1.00 | 26.46 | Y | C |
| ATOM | 9975 | CB | ASN | Y | 31 | 17.519 | 3.268 | 52.699 | 1.00 | 29.15 | Y | C |
| ATOM | 9976 | CG | ASN | Y | 31 | 18.334 | 2.019 | 52.903 | 1.00 | 27.49 | Y | C |
| ATOM | 9977 | OD1 | ASN | Y | 31 | 19.046 | 1.882 | 53.894 | 1.00 | 38.31 | Y | O |
| ATOM | 9978 | ND2 | ASN | Y | 31 | 18.241 | 1.095 | 51.960 | 1.00 | 32.26 | Y | N |
| ATOM | 9979 | C | ASN | Y | 31 | 16.472 | 4.757 | 50.986 | 1.00 | 28.83 | Y | C |
| ATOM | 9980 | O | ASN | Y | 31 | 15.298 | 4.453 | 51.159 | 1.00 | 31.06 | Y | O |
| ATOM | 9981 | N | TYR | Y | 32 | 16.854 | 5.958 | 50.570 | 1.00 | 27.35 | Y | N |
| ATOM | 9982 | CA | TYR | Y | 32 | 15.883 | 6.981 | 50.225 | 1.00 | 26.87 | Y | C |
| ATOM | 9983 | CB | TYR | Y | 32 | 15.328 | 6.682 | 48.831 | 1.00 | 27.19 | Y | C |
| ATOM | 9984 | CG | TYR | Y | 32 | 16.186 | 7.188 | 47.708 | 1.00 | 24.35 | Y | C |
| ATOM | 9985 | CD1 | TYR | Y | 32 | 17.208 | 6.411 | 47.181 | 1.00 | 20.00 | Y | C |
| ATOM | 9986 | CE1 | TYR | Y | 32 | 17.987 | 6.886 | 46.136 | 1.00 | 24.97 | Y | C |
| ATOM | 9987 | CD2 | TYR | Y | 32 | 15.962 | 8.450 | 47.166 | 1.00 | 20.29 | Y | C |
| ATOM | 9988 | CE2 | TYR | Y | 32 | 16.726 | 8.931 | 46.125 | 1.00 | 26.19 | Y | C |
| ATOM | 9989 | CZ | TYR | Y | 32 | 17.737 | 8.148 | 45.610 | 1.00 | 25.11 | Y | C |
| ATOM | 9990 | OH | TYR | Y | 32 | 18.485 | 8.634 | 44.563 | 1.00 | 28.91 | Y | O |
| ATOM | 9991 | C | TYR | Y | 32 | 14.717 | 7.098 | 51.184 | 1.00 | 26.31 | Y | C |
| ATOM | 9992 | O | TYR | Y | 32 | 13.587 | 7.279 | 50.733 | 1.00 | 27.33 | Y | O |
| ATOM | 9993 | N | ALA | Y | 33 | 14.988 | 6.991 | 52.489 | 1.00 | 26.78 | Y | N |
| ATOM | 9994 | CA | ALA | Y | 33 | 13.946 | 7.062 | 53.516 | 1.00 | 21.83 | Y | C |
| ATOM | 9995 | CB | ALA | Y | 33 | 14.526 | 7.461 | 54.819 | 1.00 | 17.03 | Y | C |
| ATOM | 9996 | C | ALA | Y | 33 | 12.838 | 8.019 | 53.110 | 1.00 | 21.50 | Y | C |
| ATOM | 9997 | O | ALA | Y | 33 | 13.097 | 9.144 | 52.702 | 1.00 | 18.97 | Y | O |
| ATOM | 9998 | N | ASN | Y | 34 | 11.607 | 7.534 | 53.220 | 1.00 | 25.22 | Y | N |
| ATOM | 9999 | CA | ASN | Y | 34 | 10.409 | 8.264 | 52.826 | 1.00 | 27.09 | Y | C |
| ATOM | 10000 | CB | ASN | Y | 34 | 9.879 | 7.545 | 51.576 | 1.00 | 36.00 | Y | C |
| ATOM | 10001 | CG | ASN | Y | 34 | 8.988 | 8.401 | 50.724 | 1.00 | 39.44 | Y | C |
| ATOM | 10002 | OD1 | ASN | Y | 34 | 9.376 | 9.472 | 50.275 | 1.00 | 41.98 | Y | O |
| ATOM | 10003 | ND2 | ASN | Y | 34 | 7.796 | 7.895 | 50.438 | 1.00 | 48.21 | Y | N |
| ATOM | 10004 | C | ASN | Y | 34 | 9.364 | 8.204 | 53.961 | 1.00 | 28.16 | Y | C |
| ATOM | 10005 | O | ASN | Y | 34 | 9.094 | 7.126 | 54.496 | 1.00 | 30.04 | Y | O |
| ATOM | 10006 | N | TRP | Y | 35 | 8.815 | 9.351 | 54.365 | 1.00 | 28.63 | Y | N |
| ATOM | 10007 | CA | TRP | Y | 35 | 7.794 | 9.369 | 55.417 | 1.00 | 25.09 | Y | C |
| ATOM | 10008 | CB | TRP | Y | 35 | 8.186 | 10.278 | 56.589 | 1.00 | 28.62 | Y | C |
| ATOM | 10009 | CG | TRP | Y | 35 | 9.398 | 9.819 | 57.331 | 1.00 | 24.88 | Y | C |
| ATOM | 10010 | CD2 | TRP | Y | 35 | 9.432 | 9.062 | 58.545 | 1.00 | 26.22 | Y | C |
| ATOM | 10011 | CE2 | TRP | Y | 35 | 10.792 | 8.859 | 58.872 | 1.00 | 24.19 | Y | C |
| ATOM | 10012 | CE3 | TRP | Y | 35 | 8.448 | 8.529 | 59.388 | 1.00 | 22.83 | Y | C |
| ATOM | 10013 | CD1 | TRP | Y | 35 | 10.695 | 10.037 | 56.983 | 1.00 | 26.91 | Y | C |
| ATOM | 10014 | NE1 | TRP | Y | 35 | 11.539 | 9.466 | 57.901 | 1.00 | 22.03 | Y | N |
| ATOM | 10015 | CZ2 | TRP | Y | 35 | 11.194 | 8.152 | 60.000 | 1.00 | 19.60 | Y | C |
| ATOM | 10016 | CZ3 | TRP | Y | 35 | 8.851 | 7.823 | 60.508 | 1.00 | 26.14 | Y | C |
| ATOM | 10017 | CH2 | TRP | Y | 35 | 10.213 | 7.642 | 60.804 | 1.00 | 20.14 | Y | C |
| ATOM | 10018 | C | TRP | Y | 35 | 6.465 | 9.829 | 54.850 | 1.00 | 25.91 | Y | C |
| ATOM | 10019 | O | TRP | Y | 35 | 6.385 | 10.854 | 54.178 | 1.00 | 22.39 | Y | O |
| ATOM | 10020 | N | VAL | Y | 36 | 5.418 | 9.078 | 55.171 | 1.00 | 23.64 | Y | N |
| ATOM | 10021 | CA | VAL | Y | 36 | 4.065 | 9.369 | 54.703 | 1.00 | 22.30 | Y | C |
| ATOM | 10022 | CB | VAL | Y | 36 | 3.601 | 8.243 | 53.701 | 1.00 | 22.35 | Y | C |
| ATOM | 10023 | CG1 | VAL | Y | 36 | 2.160 | 8.395 | 53.356 | 1.00 | 21.73 | Y | C |
| ATOM | 10024 | CG2 | VAL | Y | 36 | 4.434 | 8.302 | 52.420 | 1.00 | 19.29 | Y | C |
| ATOM | 10025 | C | VAL | Y | 36 | 3.092 | 9.509 | 55.904 | 1.00 | 20.76 | Y | C |
| ATOM | 10026 | O | VAL | Y | 36 | 3.139 | 8.746 | 56.873 | 1.00 | 20.06 | Y | O |
| ATOM | 10027 | N | GLN | Y | 37 | 2.209 | 10.490 | 55.817 | 1.00 | 22.18 | Y | N |
| ATOM | 10028 | CA | GLN | Y | 37 | 1.231 | 10.772 | 56.865 | 1.00 | 27.39 | Y | C |
| ATOM | 10029 | CB | GLN | Y | 37 | 1.214 | 12.289 | 57.086 | 1.00 | 28.67 | Y | C |
| ATOM | 10030 | CG | GLN | Y | 37 | 0.139 | 12.829 | 57.997 | 1.00 | 39.11 | Y | C |
| ATOM | 10031 | CD | GLN | Y | 37 | 0.140 | 14.350 | 58.066 | 1.00 | 33.70 | Y | C |
| ATOM | 10032 | OE1 | GLN | Y | 37 | −0.526 | 15.012 | 57.285 | 1.00 | 40.05 | Y | O |
| ATOM | 10033 | NE2 | GLN | Y | 37 | 0.879 | 14.903 | 59.018 | 1.00 | 40.30 | Y | N |
| ATOM | 10034 | C | GLN | Y | 37 | −0.170 | 10.269 | 56.476 | 1.00 | 27.42 | Y | C |
| ATOM | 10035 | O | GLN | Y | 37 | −0.691 | 10.660 | 55.437 | 1.00 | 32.10 | Y | O |
| ATOM | 10036 | N | GLU | Y | 38 | −0.739 | 9.349 | 57.255 | 1.00 | 25.85 | Y | N |
| ATOM | 10037 | CA | GLU | Y | 38 | −2.089 | 8.852 | 56.973 | 1.00 | 27.11 | Y | C |
| ATOM | 10038 | CB | GLU | Y | 38 | −2.187 | 7.327 | 57.034 | 1.00 | 27.58 | Y | C |
| ATOM | 10039 | CG | GLU | Y | 38 | −3.642 | 6.838 | 56.931 | 1.00 | 27.35 | Y | C |
| ATOM | 10040 | CD | GLU | Y | 38 | −3.811 | 5.351 | 57.241 | 1.00 | 39.41 | Y | C |
| ATOM | 10041 | OE1 | GLU | Y | 38 | −3.075 | 4.807 | 58.089 | 1.00 | 43.73 | Y | O |
| ATOM | 10042 | OE2 | GLU | Y | 38 | −4.694 | 4.714 | 56.637 | 1.00 | 44.74 | Y | O |
| ATOM | 10043 | C | GLU | Y | 38 | −3.098 | 9.444 | 57.951 | 1.00 | 28.74 | Y | C |
| ATOM | 10044 | O | GLU | Y | 38 | −3.134 | 9.076 | 59.133 | 1.00 | 27.99 | Y | O |
| ATOM | 10045 | N | LYS | Y | 39 | −3.899 | 10.379 | 57.454 | 1.00 | 28.82 | Y | N |
| ATOM | 10046 | CA | LYS | Y | 39 | −4.929 | 11.019 | 58.262 | 1.00 | 38.51 | Y | C |
| ATOM | 10047 | CB | LYS | Y | 39 | −5.197 | 12.422 | 57.730 | 1.00 | 37.38 | Y | C |
| ATOM | 10048 | CG | LYS | Y | 39 | −4.174 | 13.461 | 58.127 | 1.00 | 39.10 | Y | C |
| ATOM | 10049 | CD | LYS | Y | 39 | −4.327 | 13.835 | 59.583 | 1.00 | 44.25 | Y | C |
| ATOM | 10050 | CE | LYS | Y | 39 | −3.609 | 15.132 | 59.880 | 1.00 | 48.63 | Y | C |
| ATOM | 10051 | NZ | LYS | Y | 39 | −4.170 | 16.289 | 59.121 | 1.00 | 49.11 | Y | N |
| ATOM | 10052 | C | LYS | Y | 39 | −6.225 | 10.209 | 58.217 | 1.00 | 41.88 | Y | C |
| ATOM | 10053 | O | LYS | Y | 39 | −6.415 | 9.390 | 57.319 | 1.00 | 46.41 | Y | O |
| ATOM | 10054 | N | PRO | Y | 40 | −7.121 | 10.404 | 59.203 | 1.00 | 50.62 | Y | N |
| ATOM | 10055 | CD | PRO | Y | 40 | −7.028 | 11.295 | 60.374 | 1.00 | 50.14 | Y | C |
| ATOM | 10056 | CA | PRO | Y | 40 | −8.393 | 9.665 | 59.218 | 1.00 | 50.12 | Y | C |
| ATOM | 10057 | CB | PRO | Y | 40 | −9.135 | 10.276 | 60.420 | 1.00 | 50.36 | Y | C |
| ATOM | 10058 | CG | PRO | Y | 40 | −8.479 | 11.624 | 60.615 | 1.00 | 50.09 | Y | C |
| ATOM | 10059 | C | PRO | Y | 40 | −9.173 | 9.827 | 57.906 | 1.00 | 46.18 | Y | C |
| ATOM | 10060 | O | PRO | Y | 40 | −9.244 | 10.923 | 57.351 | 1.00 | 40.95 | Y | O |
| ATOM | 10061 | N | ASP | Y | 41 | −9.767 | 8.720 | 57.454 | 1.00 | 49.58 | Y | N |
| ATOM | 10062 | CA | ASP | Y | 41 | −10.545 | 8.608 | 56.209 | 1.00 | 49.52 | Y | C |
| ATOM | 10063 | CB | ASP | Y | 41 | −11.333 | 9.898 | 55.872 | 1.00 | 54.63 | Y | C |
| ATOM | 10064 | CG | ASP | Y | 41 | −12.330 | 10.309 | 56.982 | 1.00 | 61.97 | Y | C |
| ATOM | 10065 | OD1 | ASP | Y | 41 | −13.061 | 9.436 | 57.518 | 1.00 | 61.89 | Y | O |
| ATOM | 10066 | OD2 | ASP | Y | 41 | −12.379 | 11.519 | 57.319 | 1.00 | 60.64 | Y | O |
| ATOM | 10067 | C | ASP | Y | 41 | −9.581 | 8.212 | 55.075 | 1.00 | 47.29 | Y | C |
| ATOM | 10068 | O | ASP | Y | 41 | −9.807 | 8.496 | 53.894 | 1.00 | 41.60 | Y | O |
| ATOM | 10069 | N | HIS | Y | 42 | −8.505 | 7.536 | 55.469 | 1.00 | 40.64 | Y | N |
| ATOM | 10070 | CA | HIS | Y | 42 | −7.479 | 7.058 | 54.550 | 1.00 | 41.94 | Y | C |
| ATOM | 10071 | CB | HIS | Y | 42 | −7.994 | 5.838 | 53.784 | 1.00 | 43.42 | Y | C |
| ATOM | 10072 | CG | HIS | Y | 42 | −8.287 | 4.664 | 54.665 | 1.00 | 48.41 | Y | C |
| ATOM | 10073 | CD2 | HIS | Y | 42 | −9.406 | 3.915 | 54.817 | 1.00 | 52.54 | Y | C |
| ATOM | 10074 | ND1 | HIS | Y | 42 | −7.366 | 4.156 | 55.556 | 1.00 | 49.27 | Y | N |
| ATOM | 10075 | CE1 | HIS | Y | 42 | −7.902 | 3.147 | 56.219 | 1.00 | 51.19 | Y | C |
| ATOM | 10076 | NE2 | HIS | Y | 42 | −9.141 | 2.979 | 55.790 | 1.00 | 54.77 | Y | N |
| ATOM | 10077 | C | HIS | Y | 42 | −6.916 | 8.119 | 53.607 | 1.00 | 38.24 | Y | C |
| ATOM | 10078 | O | HIS | Y | 42 | −6.890 | 7.936 | 52.391 | 1.00 | 37.62 | Y | O |
| ATOM | 10079 | N | LEU | Y | 43 | −6.466 | 9.234 | 54.183 | 1.00 | 34.54 | Y | N |
| ATOM | 10080 | CA | LEU | Y | 43 | −5.888 | 10.325 | 53.404 | 1.00 | 29.20 | Y | C |
| ATOM | 10081 | CB | LEU | Y | 43 | −6.525 | 11.655 | 53.796 | 1.00 | 33.45 | Y | C |
| ATOM | 10082 | CG | LEU | Y | 43 | −8.061 | 11.752 | 53.708 | 1.00 | 40.37 | Y | C |
| ATOM | 10083 | CD1 | LEU | Y | 43 | −8.521 | 13.074 | 54.321 | 1.00 | 40.37 | Y | C |
| ATOM | 10084 | CD2 | LEU | Y | 43 | −8.557 | 11.622 | 52.273 | 1.00 | 33.73 | Y | C |
| ATOM | 10085 | C | LEU | Y | 43 | −4.365 | 10.391 | 53.580 | 1.00 | 31.15 | Y | C |
| ATOM | 10086 | O | LEU | Y | 43 | −3.865 | 10.852 | 54.606 | 1.00 | 31.01 | Y | O |
| ATOM | 10087 | N | PHE | Y | 44 | −3.640 | 9.888 | 52.584 | 1.00 | 27.33 | Y | N |
| ATOM | 10088 | CA | PHE | Y | 44 | −2.189 | 9.889 | 52.598 | 1.00 | 24.69 | Y | C |
| ATOM | 10089 | CB | PHE | Y | 44 | −1.641 | 8.671 | 51.866 | 1.00 | 23.46 | Y | C |
| ATOM | 10090 | CG | PHE | Y | 44 | −1.831 | 7.391 | 52.612 | 1.00 | 22.44 | Y | C |
| ATOM | 10091 | CD1 | PHE | Y | 44 | −3.028 | 6.690 | 52.524 | 1.00 | 23.27 | Y | C |
| ATOM | 10092 | CD2 | PHE | Y | 44 | −0.829 | 6.900 | 53.429 | 1.00 | 20.24 | Y | C |
| ATOM | 10093 | CE1 | PHE | Y | 44 | −3.217 | 5.514 | 53.252 | 1.00 | 23.55 | Y | C |
| ATOM | 10094 | CE2 | PHE | Y | 44 | −1.004 | 5.729 | 54.159 | 1.00 | 24.73 | Y | C |
| ATOM | 10095 | CZ | PHE | Y | 44 | −2.202 | 5.037 | 54.071 | 1.00 | 23.66 | Y | C |
| ATOM | 10096 | C | PHE | Y | 44 | −1.610 | 11.144 | 51.980 | 1.00 | 26.52 | Y | C |
| ATOM | 10097 | O | PHE | Y | 44 | −2.086 | 11.618 | 50.947 | 1.00 | 30.73 | Y | O |
| ATOM | 10098 | N | THR | Y | 45 | −0.535 | 11.624 | 52.602 | 1.00 | 29.13 | Y | N |
| ATOM | 10099 | CA | THR | Y | 45 | 0.200 | 12.828 | 52.202 | 1.00 | 28.41 | Y | C |
| ATOM | 10100 | CB | THR | Y | 45 | −0.317 | 14.080 | 52.991 | 1.00 | 30.33 | Y | C |
| ATOM | 10101 | OG1 | THR | Y | 45 | −1.387 | 14.701 | 52.266 | 1.00 | 31.90 | Y | O |
| ATOM | 10102 | CG2 | THR | Y | 45 | 0.796 | 15.106 | 53.238 | 1.00 | 37.31 | Y | C |
| ATOM | 10103 | C | THR | Y | 45 | 1.677 | 12.600 | 52.532 | 1.00 | 29.83 | Y | C |
| ATOM | 10104 | O | THR | Y | 45 | 2.013 | 12.189 | 53.652 | 1.00 | 29.44 | Y | O |
| ATOM | 10105 | N | GLY | Y | 46 | 2.543 | 12.813 | 51.543 | 1.00 | 24.18 | Y | N |
| ATOM | 10106 | CA | GLY | Y | 46 | 3.971 | 12.641 | 51.752 | 1.00 | 25.66 | Y | C |
| ATOM | 10107 | C | GLY | Y | 46 | 4.525 | 13.740 | 52.637 | 1.00 | 24.93 | Y | C |
| ATOM | 10108 | O | GLY | Y | 46 | 4.156 | 14.905 | 52.496 | 1.00 | 23.50 | Y | O |
| ATOM | 10109 | N | VAL | Y | 47 | 5.422 | 13.379 | 53.544 | 1.00 | 20.55 | Y | N |
| ATOM | 10110 | CA | VAL | Y | 47 | 5.994 | 14.349 | 54.471 | 1.00 | 21.33 | Y | C |
| ATOM | 10111 | CB | VAL | Y | 47 | 6.055 | 13.782 | 55.906 | 1.00 | 24.53 | Y | C |
| ATOM | 10112 | CG1 | VAL | Y | 47 | 6.372 | 14.896 | 56.893 | 1.00 | 17.82 | Y | C |
| ATOM | 10113 | CG2 | VAL | Y | 47 | 4.731 | 13.080 | 56.284 | 1.00 | 25.57 | Y | C |
| ATOM | 10114 | C | VAL | Y | 47 | 7.427 | 14.667 | 54.104 | 1.00 | 20.55 | Y | C |
| ATOM | 10115 | O | VAL | Y | 47 | 7.812 | 15.818 | 53.906 | 1.00 | 23.54 | Y | O |
| ATOM | 10116 | N | ILE | Y | 48 | 8.218 | 13.608 | 54.041 | 1.00 | 15.09 | Y | N |
| ATOM | 10117 | CA | ILE | Y | 48 | 9.621 | 13.692 | 53.760 | 1.00 | 15.23 | Y | C |
| ATOM | 10118 | CB | ILE | Y | 48 | 10.426 | 13.249 | 55.021 | 1.00 | 23.29 | Y | C |
| ATOM | 10119 | CG2 | ILE | Y | 48 | 11.928 | 13.062 | 54.691 | 1.00 | 19.61 | Y | C |
| ATOM | 10120 | CG1 | ILE | Y | 48 | 10.162 | 14.206 | 56.187 | 1.00 | 22.33 | Y | C |
| ATOM | 10121 | CD1 | ILE | Y | 48 | 10.611 | 15.636 | 55.922 | 1.00 | 21.48 | Y | C |
| ATOM | 10122 | C | ILE | Y | 48 | 9.960 | 12.697 | 52.687 | 1.00 | 19.69 | Y | C |
| ATOM | 10123 | O | ILE | Y | 48 | 9.456 | 11.583 | 52.701 | 1.00 | 22.69 | Y | O |
| ATOM | 10124 | N | GLY | Y | 49 | 10.893 | 13.080 | 51.823 | 1.00 | 23.46 | Y | N |
| ATOM | 10125 | CA | GLY | Y | 49 | 11.386 | 12.192 | 50.779 | 1.00 | 22.62 | Y | C |
| ATOM | 10126 | C | GLY | Y | 49 | 12.900 | 12.349 | 50.715 | 1.00 | 23.58 | Y | C |
| ATOM | 10127 | O | GLY | Y | 49 | 13.442 | 13.366 | 51.154 | 1.00 | 25.86 | Y | O |
| ATOM | 10128 | N | GLY | Y | 50 | 13.600 | 11.350 | 50.200 | 1.00 | 29.00 | Y | N |
| ATOM | 10129 | CA | GLY | Y | 50 | 15.054 | 11.451 | 50.091 | 1.00 | 31.97 | Y | C |
| ATOM | 10130 | C | GLY | Y | 50 | 15.793 | 11.694 | 51.398 | 1.00 | 31.93 | Y | C |
| ATOM | 10131 | O | GLY | Y | 50 | 16.689 | 12.538 | 51.477 | 1.00 | 29.76 | Y | O |
| ATOM | 10132 | N | THR | Y | 51 | 15.399 | 10.943 | 52.423 | 1.00 | 32.17 | Y | N |
| ATOM | 10133 | CA | THR | Y | 51 | 15.970 | 11.011 | 53.768 | 1.00 | 28.07 | Y | C |
| ATOM | 10134 | CB | THR | Y | 51 | 17.500 | 10.770 | 53.764 | 1.00 | 27.29 | Y | C |
| ATOM | 10135 | OG1 | THR | Y | 51 | 17.788 | 9.502 | 53.170 | 1.00 | 31.76 | Y | O |
| ATOM | 10136 | CG2 | THR | Y | 51 | 18.052 | 10.772 | 55.178 | 1.00 | 23.08 | Y | C |
| ATOM | 10137 | C | THR | Y | 51 | 15.687 | 12.294 | 54.541 | 1.00 | 30.05 | Y | C |
| ATOM | 10138 | O | THR | Y | 51 | 15.227 | 12.243 | 55.682 | 1.00 | 28.30 | Y | O |
| ATOM | 10139 | N | ASN | Y | 52 | 15.898 | 13.445 | 53.913 | 1.00 | 29.91 | Y | N |
| ATOM | 10140 | CA | ASN | Y | 52 | 15.717 | 14.688 | 54.641 | 1.00 | 29.89 | Y | C |
| ATOM | 10141 | CB | ASN | Y | 52 | 17.052 | 15.114 | 55.255 | 1.00 | 34.44 | Y | C |
| ATOM | 10142 | CG | ASN | Y | 52 | 18.093 | 15.503 | 54.207 | 1.00 | 35.96 | Y | C |
| ATOM | 10143 | OD1 | ASN | Y | 52 | 19.147 | 16.038 | 54.551 | 1.00 | 43.77 | Y | O |
| ATOM | 10144 | ND2 | ASN | Y | 52 | 17.803 | 15.243 | 52.930 | 1.00 | 35.75 | Y | N |
| ATOM | 10145 | C | ASN | Y | 52 | 15.149 | 15.849 | 53.879 | 1.00 | 26.89 | Y | C |
| ATOM | 10146 | O | ASN | Y | 52 | 15.451 | 16.989 | 54.211 | 1.00 | 26.77 | Y | O |
| ATOM | 10147 | N | ASN | Y | 53 | 14.340 | 15.578 | 52.863 | 1.00 | 26.91 | Y | N |
| ATOM | 10148 | CA | ASN | Y | 53 | 13.727 | 16.655 | 52.096 | 1.00 | 25.07 | Y | C |
| ATOM | 10149 | CB | ASN | Y | 53 | 13.922 | 16.462 | 50.590 | 1.00 | 21.17 | Y | C |
| ATOM | 10150 | CG | ASN | Y | 53 | 15.375 | 16.293 | 50.212 | 1.00 | 25.67 | Y | C |
| ATOM | 10151 | OD1 | ASN | Y | 53 | 16.149 | 17.252 | 50.240 | 1.00 | 27.36 | Y | O |
| ATOM | 10152 | ND2 | ASN | Y | 53 | 15.765 | 15.060 | 49.888 | 1.00 | 21.87 | Y | N |
| ATOM | 10153 | C | ASN | Y | 53 | 12.250 | 16.756 | 52.405 | 1.00 | 27.17 | Y | C |
| ATOM | 10154 | O | ASN | Y | 53 | 11.502 | 15.784 | 52.269 | 1.00 | 35.75 | Y | O |
| ATOM | 10155 | N | ARG | Y | 54 | 11.849 | 17.934 | 52.851 | 1.00 | 26.84 | Y | N |
| ATOM | 10156 | CA | ARG | Y | 54 | 10.461 | 18.214 | 53.156 | 1.00 | 31.71 | Y | C |
| ATOM | 10157 | CB | ARG | Y | 54 | 10.354 | 19.551 | 53.876 | 1.00 | 28.28 | Y | C |
| ATOM | 10158 | CG | ARG | Y | 54 | 9.665 | 19.470 | 55.208 | 1.00 | 39.62 | Y | C |
| ATOM | 10159 | CD | ARG | Y | 54 | 9.525 | 20.841 | 55.852 | 1.00 | 42.47 | Y | C |
| ATOM | 10160 | NE | ARG | Y | 54 | 10.812 | 21.480 | 56.102 | 1.00 | 44.75 | Y | N |
| ATOM | 10161 | CZ | ARG | Y | 54 | 11.151 | 22.668 | 55.614 | 1.00 | 50.81 | Y | C |
| ATOM | 10162 | NH1 | ARG | Y | 54 | 10.298 | 23.348 | 54.853 | 1.00 | 47.57 | Y | N |
| ATOM | 10163 | NH2 | ARG | Y | 54 | 12.345 | 23.178 | 55.882 | 1.00 | 57.03 | Y | N |
| ATOM | 10164 | C | ARG | Y | 54 | 9.781 | 18.346 | 51.811 | 1.00 | 34.27 | Y | C |
| ATOM | 10165 | O | ARG | Y | 54 | 10.284 | 19.055 | 50.933 | 1.00 | 41.03 | Y | O |
| ATOM | 10166 | N | ALA | Y | 55 | 8.661 | 17.660 | 51.618 | 1.00 | 30.58 | Y | N |
| ATOM | 10167 | CA | ALA | Y | 55 | 7.956 | 17.774 | 50.341 | 1.00 | 23.38 | Y | C |
| ATOM | 10168 | CB | ALA | Y | 55 | 6.897 | 16.695 | 50.220 | 1.00 | 19.82 | Y | C |
| ATOM | 10169 | C | ALA | Y | 55 | 7.326 | 19.170 | 50.273 | 1.00 | 20.42 | Y | C |
| ATOM | 10170 | O | ALA | Y | 55 | 6.970 | 19.744 | 51.299 | 1.00 | 26.45 | Y | O |
| ATOM | 10171 | N | PRO | Y | 56 | 7.198 | 19.741 | 49.067 | 1.00 | 16.57 | Y | N |
| ATOM | 10172 | CD | PRO | Y | 56 | 7.430 | 19.132 | 47.742 | 1.00 | 14.49 | Y | C |
| ATOM | 10173 | CA | PRO | Y | 56 | 6.602 | 21.079 | 48.939 | 1.00 | 20.35 | Y | C |
| ATOM | 10174 | CB | PRO | Y | 56 | 6.378 | 21.212 | 47.427 | 1.00 | 16.97 | Y | C |
| ATOM | 10175 | CG | PRO | Y | 56 | 7.460 | 20.329 | 46.837 | 1.00 | 15.64 | Y | C |
| ATOM | 10176 | C | PRO | Y | 56 | 5.276 | 21.214 | 49.708 | 1.00 | 27.32 | Y | C |
| ATOM | 10177 | O | PRO | Y | 56 | 4.448 | 20.294 | 49.710 | 1.00 | 29.12 | Y | O |
| ATOM | 10178 | N | GLY | Y | 57 | 5.100 | 22.348 | 50.389 | 1.00 | 32.63 | Y | N |
| ATOM | 10179 | CA | GLY | Y | 57 | 3.873 | 22.586 | 51.142 | 1.00 | 38.03 | Y | C |
| ATOM | 10180 | C | GLY | Y | 57 | 3.906 | 22.205 | 52.613 | 1.00 | 34.57 | Y | C |
| ATOM | 10181 | O | GLY | Y | 57 | 3.197 | 22.786 | 53.425 | 1.00 | 35.23 | Y | O |
| ATOM | 10182 | N | VAL | Y | 58 | 4.749 | 21.238 | 52.946 | 1.00 | 32.53 | Y | N |
| ATOM | 10183 | CA | VAL | Y | 58 | 4.888 | 20.743 | 54.304 | 1.00 | 36.75 | Y | C |
| ATOM | 10184 | CB | VAL | Y | 58 | 5.740 | 19.458 | 54.319 | 1.00 | 29.21 | Y | C |
| ATOM | 10185 | CG1 | VAL | Y | 58 | 5.868 | 18.917 | 55.719 | 1.00 | 28.68 | Y | C |
| ATOM | 10186 | CG2 | VAL | Y | 58 | 5.109 | 18.422 | 53.411 | 1.00 | 29.24 | Y | C |
| ATOM | 10187 | C | VAL | Y | 58 | 5.505 | 21.787 | 55.235 | 1.00 | 39.81 | Y | C |
| ATOM | 10188 | O | VAL | Y | 58 | 6.552 | 22.363 | 54.920 | 1.00 | 42.80 | Y | O |
| ATOM | 10189 | N | PRO | Y | 59 | 4.859 | 22.036 | 56.396 | 1.00 | 39.80 | Y | N |
| ATOM | 10190 | CD | PRO | Y | 59 | 3.569 | 21.441 | 56.786 | 1.00 | 38.48 | Y | C |
| ATOM | 10191 | CA | PRO | Y | 59 | 5.300 | 23.004 | 57.410 | 1.00 | 41.66 | Y | C |
| ATOM | 10192 | CB | PRO | Y | 59 | 4.330 | 22.748 | 58.561 | 1.00 | 41.25 | Y | C |
| ATOM | 10193 | CG | PRO | Y | 59 | 3.071 | 22.403 | 57.844 | 1.00 | 44.80 | Y | C |
| ATOM | 10194 | C | PRO | Y | 59 | 6.747 | 22.797 | 57.844 | 1.00 | 39.23 | Y | C |
| ATOM | 10195 | O | PRO | Y | 59 | 7.215 | 21.665 | 57.934 | 1.00 | 38.96 | Y | O |
| ATOM | 10196 | N | ALA | Y | 60 | 7.436 | 23.896 | 58.139 | 1.00 | 39.26 | Y | N |
| ATOM | 10197 | CA | ALA | Y | 60 | 8.835 | 23.846 | 58.554 | 1.00 | 44.20 | Y | C |
| ATOM | 10198 | CB | ALA | Y | 60 | 9.401 | 25.259 | 58.676 | 1.00 | 39.31 | Y | C |
| ATOM | 10199 | C | ALA | Y | 60 | 9.115 | 23.048 | 59.829 | 1.00 | 43.36 | Y | C |
| ATOM | 10200 | O | ALA | Y | 60 | 10.256 | 22.657 | 60.070 | 1.00 | 49.57 | Y | O |
| ATOM | 10201 | N | ARG | Y | 61 | 8.089 | 22.786 | 60.637 | 1.00 | 42.33 | Y | N |
| ATOM | 10202 | CA | ARG | Y | 61 | 8.290 | 22.029 | 61.876 | 1.00 | 37.55 | Y | C |
| ATOM | 10203 | CB | ARG | Y | 61 | 7.095 | 22.216 | 62.819 | 1.00 | 39.21 | Y | C |
| ATOM | 10204 | CG | ARG | Y | 61 | 5.761 | 21.781 | 62.249 | 1.00 | 39.98 | Y | C |
| ATOM | 10205 | CD | ARG | Y | 61 | 4.636 | 21.979 | 63.256 | 1.00 | 38.87 | Y | C |
| ATOM | 10206 | NE | ARG | Y | 61 | 3.371 | 21.435 | 62.768 | 1.00 | 38.48 | Y | N |
| ATOM | 10207 | CZ | ARG | Y | 61 | 2.545 | 22.078 | 61.948 | 1.00 | 41.22 | Y | C |
| ATOM | 10208 | NH1 | ARG | Y | 61 | 2.838 | 23.300 | 61.525 | 1.00 | 36.77 | Y | N |
| ATOM | 10209 | NH2 | ARG | Y | 61 | 1.442 | 21.479 | 61.512 | 1.00 | 41.95 | Y | N |
| ATOM | 10210 | C | ARG | Y | 61 | 8.596 | 20.538 | 61.659 | 1.00 | 36.73 | Y | C |
| ATOM | 10211 | O | ARG | Y | 61 | 9.015 | 19.846 | 62.587 | 1.00 | 37.00 | Y | O |
| ATOM | 10212 | N | PHE | Y | 62 | 8.374 | 20.049 | 60.440 | 1.00 | 32.24 | Y | N |
| ATOM | 10213 | CA | PHE | Y | 62 | 8.639 | 18.655 | 60.087 | 1.00 | 29.06 | Y | C |
| ATOM | 10214 | CB | PHE | Y | 62 | 7.663 | 18.164 | 59.012 | 1.00 | 30.03 | Y | C |
| ATOM | 10215 | CG | PHE | Y | 62 | 6.273 | 17.940 | 59.509 | 1.00 | 31.19 | Y | C |
| ATOM | 10216 | CD1 | PHE | Y | 62 | 5.907 | 16.708 | 60.033 | 1.00 | 26.61 | Y | C |
| ATOM | 10217 | CD2 | PHE | Y | 62 | 5.316 | 18.946 | 59.419 | 1.00 | 32.70 | Y | C |
| ATOM | 10218 | CE1 | PHE | Y | 62 | 4.625 | 16.477 | 60.453 | 1.00 | 23.07 | Y | C |
| ATOM | 10219 | CE2 | PHE | Y | 62 | 4.014 | 18.722 | 59.842 | 1.00 | 29.33 | Y | C |
| ATOM | 10220 | CZ | PHE | Y | 62 | 3.672 | 17.482 | 60.359 | 1.00 | 28.79 | Y | C |
| ATOM | 10221 | C | PHE | Y | 62 | 10.024 | 18.593 | 59.490 | 1.00 | 26.17 | Y | C |
| ATOM | 10222 | O | PHE | Y | 62 | 10.371 | 19.435 | 58.671 | 1.00 | 25.16 | Y | O |
| ATOM | 10223 | N | SER | Y | 63 | 10.804 | 17.594 | 59.879 | 1.00 | 20.31 | Y | N |
| ATOM | 10224 | CA | SER | Y | 63 | 12.138 | 17.441 | 59.338 | 1.00 | 30.80 | Y | C |
| ATOM | 10225 | CB | SER | Y | 63 | 13.141 | 18.350 | 60.061 | 1.00 | 29.25 | Y | C |
| ATOM | 10226 | OG | SER | Y | 63 | 13.442 | 17.865 | 61.353 | 1.00 | 39.47 | Y | O |
| ATOM | 10227 | C | SER | Y | 63 | 12.543 | 15.994 | 59.480 | 1.00 | 26.85 | Y | C |
| ATOM | 10228 | O | SER | Y | 63 | 12.097 | 15.323 | 60.400 | 1.00 | 31.39 | Y | O |
| ATOM | 10229 | N | GLY | Y | 64 | 13.385 | 15.525 | 58.562 | 1.00 | 29.59 | Y | N |
| ATOM | 10230 | CA | GLY | Y | 64 | 13.849 | 14.148 | 58.588 | 1.00 | 24.33 | Y | C |
| ATOM | 10231 | C | GLY | Y | 64 | 15.365 | 14.071 | 58.599 | 1.00 | 28.30 | Y | C |
| ATOM | 10232 | O | GLY | Y | 64 | 16.054 | 15.005 | 58.173 | 1.00 | 32.05 | Y | O |
| ATOM | 10233 | N | SER | Y | 65 | 15.901 | 12.955 | 59.073 | 1.00 | 22.27 | Y | N |
| ATOM | 10234 | CA | SER | Y | 65 | 17.341 | 12.814 | 59.128 | 1.00 | 23.51 | Y | C |
| ATOM | 10235 | CB | SER | Y | 65 | 17.913 | 13.765 | 60.188 | 1.00 | 25.92 | Y | C |
| ATOM | 10236 | OG | SER | Y | 65 | 17.483 | 13.396 | 61.487 | 1.00 | 30.08 | Y | O |
| ATOM | 10237 | C | SER | Y | 65 | 17.721 | 11.409 | 59.496 | 1.00 | 24.93 | Y | C |
| ATOM | 10238 | O | SER | Y | 65 | 16.875 | 10.522 | 59.544 | 1.00 | 30.20 | Y | O |
| ATOM | 10239 | N | LEU | Y | 66 | 19.023 | 11.213 | 59.678 | 1.00 | 23.28 | Y | N |
| ATOM | 10240 | CA | LEU | Y | 66 | 19.579 | 9.949 | 60.120 | 1.00 | 27.64 | Y | C |
| ATOM | 10241 | CB | LEU | Y | 66 | 20.660 | 9.444 | 59.163 | 1.00 | 29.31 | Y | C |
| ATOM | 10242 | CG | LEU | Y | 66 | 20.186 | 8.942 | 57.798 | 1.00 | 34.04 | Y | C |
| ATOM | 10243 | CD1 | LEU | Y | 66 | 21.365 | 8.398 | 57.005 | 1.00 | 32.61 | Y | C |
| ATOM | 10244 | CD2 | LEU | Y | 66 | 19.127 | 7.857 | 57.981 | 1.00 | 33.79 | Y | C |
| ATOM | 10245 | C | LEU | Y | 66 | 20.172 | 10.230 | 61.506 | 1.00 | 33.08 | Y | C |
| ATOM | 10246 | O | LEU | Y | 66 | 20.955 | 11.168 | 61.685 | 1.00 | 33.76 | Y | O |
| ATOM | 10247 | N | ILE | Y | 67 | 19.684 | 9.500 | 62.503 | 1.00 | 36.20 | Y | N |
| ATOM | 10248 | CA | ILE | Y | 67 | 20.150 | 9.637 | 63.876 | 1.00 | 40.93 | Y | C |
| ATOM | 10249 | CB | ILE | Y | 67 | 19.024 | 10.072 | 64.854 | 1.00 | 41.23 | Y | C |
| ATOM | 10250 | CG2 | ILE | Y | 67 | 19.639 | 10.465 | 66.186 | 1.00 | 40.51 | Y | C |
| ATOM | 10251 | CG1 | ILE | Y | 67 | 18.180 | 11.224 | 64.280 | 1.00 | 41.00 | Y | C |
| ATOM | 10252 | CD1 | ILE | Y | 67 | 18.879 | 12.574 | 64.194 | 1.00 | 51.22 | Y | C |
| ATOM | 10253 | C | ILE | Y | 67 | 20.570 | 8.229 | 64.248 | 1.00 | 41.53 | Y | C |
| ATOM | 10254 | O | ILE | Y | 67 | 19.733 | 7.340 | 64.376 | 1.00 | 45.21 | Y | O |
| ATOM | 10255 | N | GLY | Y | 68 | 21.867 | 8.028 | 64.423 | 1.00 | 45.69 | Y | N |
| ATOM | 10256 | CA | GLY | Y | 68 | 22.345 | 6.702 | 64.747 | 1.00 | 49.96 | Y | C |
| ATOM | 10257 | C | GLY | Y | 68 | 22.249 | 5.868 | 63.485 | 1.00 | 50.39 | Y | C |
| ATOM | 10258 | O | GLY | Y | 68 | 22.578 | 6.357 | 62.395 | 1.00 | 50.02 | Y | O |
| ATOM | 10259 | N | ASP | Y | 69 | 21.754 | 4.639 | 63.627 | 1.00 | 48.55 | Y | N |
| ATOM | 10260 | CA | ASP | Y | 69 | 21.608 | 3.700 | 62.507 | 1.00 | 44.39 | Y | C |
| ATOM | 10261 | CB | ASP | Y | 69 | 22.061 | 2.292 | 62.928 | 1.00 | 43.39 | Y | C |
| ATOM | 10262 | CG | ASP | Y | 69 | 21.326 | 1.777 | 64.163 | 1.00 | 42.70 | Y | C |
| ATOM | 10263 | OD1 | ASP | Y | 69 | 21.505 | 2.363 | 65.252 | 0.00 | 41.77 | Y | O |
| ATOM | 10264 | OD2 | ASP | Y | 69 | 20.581 | 0.782 | 64.042 | 0.00 | 41.81 | Y | O |
| ATOM | 10265 | C | ASP | Y | 69 | 20.195 | 3.639 | 61.925 | 1.00 | 40.54 | Y | C |
| ATOM | 10266 | O | ASP | Y | 69 | 19.810 | 2.647 | 61.294 | 1.00 | 36.72 | Y | O |
| ATOM | 10267 | N | LYS | Y | 70 | 19.421 | 4.700 | 62.126 | 1.00 | 38.88 | Y | N |
| ATOM | 10268 | CA | LYS | Y | 70 | 18.073 | 4.723 | 61.598 | 1.00 | 31.29 | Y | C |
| ATOM | 10269 | CB | LYS | Y | 70 | 17.091 | 4.054 | 62.573 | 1.00 | 41.32 | Y | C |
| ATOM | 10270 | CG | LYS | Y | 70 | 17.027 | 4.587 | 63.996 | 1.00 | 41.67 | Y | C |
| ATOM | 10271 | CD | LYS | Y | 70 | 16.120 | 3.664 | 64.833 | 1.00 | 43.53 | Y | C |
| ATOM | 10272 | CE | LYS | Y | 70 | 16.016 | 4.105 | 66.296 | 1.00 | 42.66 | Y | C |
| ATOM | 10273 | NZ | LYS | Y | 70 | 15.437 | 3.024 | 67.149 | 1.00 | 40.55 | Y | N |
| ATOM | 10274 | C | LYS | Y | 70 | 17.587 | 6.092 | 61.152 | 1.00 | 29.92 | Y | C |
| ATOM | 10275 | O | LYS | Y | 70 | 18.162 | 7.110 | 61.523 | 1.00 | 28.71 | Y | O |
| ATOM | 10276 | N | ALA | Y | 71 | 16.575 | 6.102 | 60.288 | 1.00 | 21.92 | Y | N |
| ATOM | 10277 | CA | ALA | Y | 71 | 16.017 | 7.345 | 59.790 | 1.00 | 22.17 | Y | C |
| ATOM | 10278 | CB | ALA | Y | 71 | 15.303 | 7.125 | 58.480 | 1.00 | 24.23 | Y | C |
| ATOM | 10279 | C | ALA | Y | 71 | 15.052 | 7.865 | 60.839 | 1.00 | 22.67 | Y | C |
| ATOM | 10280 | O | ALA | Y | 71 | 14.504 | 7.094 | 61.623 | 1.00 | 20.10 | Y | O |
| ATOM | 10281 | N | ALA | Y | 72 | 14.830 | 9.170 | 60.841 | 1.00 | 25.48 | Y | N |
| ATOM | 10282 | CA | ALA | Y | 72 | 13.954 | 9.777 | 61.825 | 1.00 | 25.79 | Y | C |
| ATOM | 10283 | CB | ALA | Y | 72 | 14.752 | 10.196 | 63.041 | 1.00 | 26.87 | Y | C |
| ATOM | 10284 | C | ALA | Y | 72 | 13.200 | 10.961 | 61.299 | 1.00 | 26.07 | Y | C |
| ATOM | 10285 | O | ALA | Y | 72 | 13.678 | 11.699 | 60.438 | 1.00 | 25.12 | Y | O |
| ATOM | 10286 | N | LEU | Y | 73 | 12.011 | 11.137 | 61.855 | 1.00 | 26.29 | Y | N |
| ATOM | 10287 | CA | LEU | Y | 73 | 11.130 | 12.231 | 61.497 | 1.00 | 25.27 | Y | C |
| ATOM | 10288 | CB | LEU | Y | 73 | 9.810 | 11.690 | 60.943 | 1.00 | 26.43 | Y | C |
| ATOM | 10289 | CG | LEU | Y | 73 | 8.674 | 12.686 | 60.722 | 1.00 | 26.63 | Y | C |
| ATOM | 10290 | CD1 | LEU | Y | 73 | 9.055 | 13.771 | 59.732 | 1.00 | 23.23 | Y | C |
| ATOM | 10291 | CD2 | LEU | Y | 73 | 7.475 | 11.937 | 60.239 | 1.00 | 24.61 | Y | C |
| ATOM | 10292 | C | LEU | Y | 73 | 10.904 | 12.972 | 62.795 | 1.00 | 27.66 | Y | C |
| ATOM | 10293 | O | LEU | Y | 73 | 10.412 | 12.403 | 63.777 | 1.00 | 33.76 | Y | O |
| ATOM | 10294 | N | THR | Y | 74 | 11.303 | 14.235 | 62.811 | 1.00 | 29.57 | Y | N |
| ATOM | 10295 | CA | THR | Y | 74 | 11.165 | 15.054 | 63.997 | 1.00 | 22.17 | Y | C |
| ATOM | 10296 | CB | THR | Y | 74 | 12.526 | 15.710 | 64.385 | 1.00 | 28.05 | Y | C |
| ATOM | 10297 | OG1 | THR | Y | 74 | 13.476 | 14.681 | 64.700 | 1.00 | 26.39 | Y | O |
| ATOM | 10298 | CG2 | THR | Y | 74 | 12.369 | 16.636 | 65.602 | 1.00 | 26.43 | Y | C |
| ATOM | 10299 | C | THR | Y | 74 | 10.105 | 16.110 | 63.767 | 1.00 | 25.46 | Y | C |
| ATOM | 10300 | O | THR | Y | 74 | 10.033 | 16.730 | 62.712 | 1.00 | 23.06 | Y | O |
| ATOM | 10301 | N | ILE | Y | 75 | 9.220 | 16.250 | 64.737 | 1.00 | 28.76 | Y | N |
| ATOM | 10302 | CA | ILE | Y | 75 | 8.180 | 17.246 | 64.646 | 1.00 | 29.58 | Y | C |
| ATOM | 10303 | CB | ILE | Y | 75 | 6.788 | 16.619 | 64.814 | 1.00 | 20.66 | Y | C |
| ATOM | 10304 | CG2 | ILE | Y | 75 | 5.749 | 17.662 | 64.579 | 1.00 | 16.90 | Y | C |
| ATOM | 10305 | CG1 | ILE | Y | 75 | 6.599 | 15.483 | 63.808 | 1.00 | 18.59 | Y | C |
| ATOM | 10306 | CD1 | ILE | Y | 75 | 5.242 | 14.768 | 63.891 | 1.00 | 17.15 | Y | C |
| ATOM | 10307 | C | ILE | Y | 75 | 8.493 | 18.231 | 65.775 | 1.00 | 37.98 | Y | C |
| ATOM | 10308 | O | ILE | Y | 75 | 8.448 | 17.880 | 66.960 | 1.00 | 42.07 | Y | O |
| ATOM | 10309 | N | THR | Y | 76 | 8.888 | 19.439 | 65.394 | 1.00 | 37.48 | Y | N |
| ATOM | 10310 | CA | THR | Y | 76 | 9.248 | 20.464 | 66.354 | 1.00 | 37.33 | Y | C |
| ATOM | 10311 | CB | THR | Y | 76 | 10.510 | 21.191 | 65.894 | 1.00 | 40.23 | Y | C |
| ATOM | 10312 | OG1 | THR | Y | 76 | 11.590 | 20.251 | 65.889 | 1.00 | 41.01 | Y | O |
| ATOM | 10313 | CG2 | THR | Y | 76 | 10.850 | 22.356 | 66.817 | 1.00 | 36.23 | Y | C |
| ATOM | 10314 | C | THR | Y | 76 | 8.117 | 21.430 | 66.633 | 1.00 | 37.87 | Y | C |
| ATOM | 10315 | O | THR | Y | 76 | 7.958 | 22.457 | 65.956 | 1.00 | 39.73 | Y | O |
| ATOM | 10316 | N | GLY | Y | 77 | 7.369 | 21.109 | 67.687 | 1.00 | 40.76 | Y | N |
| ATOM | 10317 | CA | GLY | Y | 77 | 6.229 | 21.914 | 68.081 | 1.00 | 32.11 | Y | C |
| ATOM | 10318 | C | GLY | Y | 77 | 5.017 | 21.272 | 67.455 | 1.00 | 28.19 | Y | C |
| ATOM | 10319 | O | GLY | Y | 77 | 4.344 | 21.886 | 66.634 | 1.00 | 30.59 | Y | O |
| ATOM | 10320 | N | ALA | Y | 78 | 4.772 | 20.016 | 67.821 | 1.00 | 30.76 | Y | N |
| ATOM | 10321 | CA | ALA | Y | 78 | 3.645 | 19.245 | 67.306 | 1.00 | 27.87 | Y | C |
| ATOM | 10322 | CB | ALA | Y | 78 | 3.593 | 17.897 | 67.961 | 1.00 | 28.57 | Y | C |
| ATOM | 10323 | C | ALA | Y | 78 | 2.321 | 19.952 | 67.523 | 1.00 | 36.11 | Y | C |
| ATOM | 10324 | O | ALA | Y | 78 | 2.012 | 20.367 | 68.635 | 1.00 | 35.57 | Y | O |
| ATOM | 10325 | N | GLN | Y | 79 | 1.549 | 20.113 | 66.454 | 1.00 | 35.02 | Y | N |
| ATOM | 10326 | CA | GLN | Y | 79 | 0.251 | 20.744 | 66.571 | 1.00 | 36.71 | Y | C |
| ATOM | 10327 | CB | GLN | Y | 79 | 0.031 | 21.741 | 65.435 | 1.00 | 39.23 | Y | C |
| ATOM | 10328 | CG | GLN | Y | 79 | 0.870 | 23.011 | 65.594 | 1.00 | 46.69 | Y | C |
| ATOM | 10329 | CD | GLN | Y | 79 | 0.646 | 24.037 | 64.486 | 1.00 | 51.70 | Y | C |
| ATOM | 10330 | OE1 | GLN | Y | 79 | 1.548 | 24.812 | 64.158 | 1.00 | 53.43 | Y | O |
| ATOM | 10331 | NE2 | GLN | Y | 79 | −0.558 | 24.058 | 63.918 | 1.00 | 55.04 | Y | N |
| ATOM | 10332 | C | GLN | Y | 79 | −0.809 | 19.655 | 66.593 | 1.00 | 40.87 | Y | C |
| ATOM | 10333 | O | GLN | Y | 79 | −0.493 | 18.483 | 66.373 | 1.00 | 48.01 | Y | O |
| ATOM | 10334 | N | THR | Y | 80 | −2.047 | 20.019 | 66.921 | 1.00 | 45.09 | Y | N |
| ATOM | 10335 | CA | THR | Y | 80 | −3.136 | 19.043 | 66.968 | 1.00 | 46.79 | Y | C |
| ATOM | 10336 | CB | THR | Y | 80 | −4.429 | 19.669 | 67.522 | 1.00 | 51.15 | Y | C |
| ATOM | 10337 | OG1 | THR | Y | 80 | −4.216 | 20.060 | 68.888 | 1.00 | 56.75 | Y | O |
| ATOM | 10338 | CG2 | THR | Y | 80 | −5.581 | 18.662 | 67.474 | 1.00 | 54.61 | Y | C |
| ATOM | 10339 | C | THR | Y | 80 | −3.390 | 18.448 | 65.580 | 1.00 | 46.08 | Y | C |
| ATOM | 10340 | O | THR | Y | 80 | −3.671 | 17.252 | 65.440 | 1.00 | 42.93 | Y | O |
| ATOM | 10341 | N | GLU | Y | 81 | −3.239 | 19.279 | 64.555 | 1.00 | 41.64 | Y | N |
| ATOM | 10342 | CA | GLU | Y | 81 | −3.429 | 18.839 | 63.181 | 1.00 | 44.75 | Y | C |
| ATOM | 10343 | CB | GLU | Y | 81 | −3.373 | 20.035 | 62.221 | 1.00 | 47.17 | Y | C |
| ATOM | 10344 | CG | GLU | Y | 81 | −2.259 | 21.023 | 62.535 | 1.00 | 55.72 | Y | C |
| ATOM | 10345 | CD | GLU | Y | 81 | −2.177 | 22.166 | 61.539 | 1.00 | 60.29 | Y | C |
| ATOM | 10346 | OE1 | GLU | Y | 81 | −1.538 | 21.974 | 60.477 | 1.00 | 59.22 | Y | O |
| ATOM | 10347 | OE2 | GLU | Y | 81 | −2.739 | 23.254 | 61.822 | 1.00 | 61.87 | Y | O |
| ATOM | 10348 | C | GLU | Y | 81 | −2.416 | 17.769 | 62.764 | 1.00 | 41.59 | Y | C |
| ATOM | 10349 | O | GLU | Y | 81 | −2.602 | 17.109 | 61.749 | 1.00 | 47.58 | Y | O |
| ATOM | 10350 | N | ASP | Y | 82 | −1.373 | 17.572 | 63.566 | 1.00 | 33.71 | Y | N |
| ATOM | 10351 | CA | ASP | Y | 82 | −0.351 | 16.583 | 63.256 | 1.00 | 26.31 | Y | C |
| ATOM | 10352 | CB | ASP | Y | 82 | 1.017 | 17.007 | 63.824 | 1.00 | 35.24 | Y | C |
| ATOM | 10353 | CG | ASP | Y | 82 | 1.480 | 18.371 | 63.329 | 1.00 | 37.47 | Y | C |
| ATOM | 10354 | OD1 | ASP | Y | 82 | 0.818 | 18.977 | 62.455 | 1.00 | 38.84 | Y | O |
| ATOM | 10355 | OD2 | ASP | Y | 82 | 2.526 | 18.842 | 63.831 | 1.00 | 36.17 | Y | O |
| ATOM | 10356 | C | ASP | Y | 82 | −0.673 | 15.177 | 63.736 | 1.00 | 25.43 | Y | C |
| ATOM | 10357 | O | ASP | Y | 82 | 0.135 | 14.266 | 63.540 | 1.00 | 25.07 | Y | O |
| ATOM | 10358 | N | GLU | Y | 83 | −1.803 | 15.001 | 64.421 | 1.00 | 29.27 | Y | N |
| ATOM | 10359 | CA | GLU | Y | 83 | −2.208 | 13.669 | 64.895 | 1.00 | 30.83 | Y | C |
| ATOM | 10360 | CB | GLU | Y | 83 | −3.484 | 13.761 | 65.739 | 1.00 | 37.60 | Y | C |
| ATOM | 10361 | CG | GLU | Y | 83 | −3.966 | 12.425 | 66.342 | 1.00 | 44.06 | Y | C |
| ATOM | 10362 | CD | GLU | Y | 83 | −5.216 | 12.561 | 67.215 | 1.00 | 45.97 | Y | C |
| ATOM | 10363 | OE1 | GLU | Y | 83 | −5.540 | 13.690 | 67.639 | 1.00 | 53.76 | Y | O |
| ATOM | 10364 | OE2 | GLU | Y | 83 | −5.879 | 11.532 | 67.488 | 1.00 | 55.95 | Y | O |
| ATOM | 10365 | C | GLU | Y | 83 | −2.472 | 12.840 | 63.638 | 1.00 | 28.90 | Y | C |
| ATOM | 10366 | O | GLU | Y | 83 | −3.201 | 13.273 | 62.750 | 1.00 | 30.39 | Y | O |
| ATOM | 10367 | N | ALA | Y | 84 | −1.829 | 11.682 | 63.547 | 1.00 | 26.33 | Y | N |
| ATOM | 10368 | CA | ALA | Y | 84 | −1.964 | 10.822 | 62.382 | 1.00 | 20.84 | Y | C |
| ATOM | 10369 | CB | ALA | Y | 84 | −1.476 | 11.575 | 61.139 | 1.00 | 22.60 | Y | C |
| ATOM | 10370 | C | ALA | Y | 84 | −1.129 | 9.563 | 62.544 | 1.00 | 22.02 | Y | C |
| ATOM | 10371 | O | ALA | Y | 84 | −0.510 | 9.327 | 63.598 | 1.00 | 21.74 | Y | O |
| ATOM | 10372 | N | ILE | Y | 85 | −1.149 | 8.743 | 61.498 | 1.00 | 15.99 | Y | N |
| ATOM | 10373 | CA | ILE | Y | 85 | −0.324 | 7.548 | 61.452 | 1.00 | 22.71 | Y | C |
| ATOM | 10374 | CB | ILE | Y | 85 | −1.112 | 6.323 | 60.946 | 1.00 | 22.59 | Y | C |
| ATOM | 10375 | CG2 | ILE | Y | 85 | −0.200 | 5.079 | 60.945 | 1.00 | 21.24 | Y | C |
| ATOM | 10376 | CG1 | ILE | Y | 85 | −2.313 | 6.074 | 61.862 | 1.00 | 24.03 | Y | C |
| ATOM | 10377 | CD1 | ILE | Y | 85 | −3.142 | 4.878 | 61.474 | 1.00 | 34.71 | Y | C |
| ATOM | 10378 | C | ILE | Y | 85 | 0.819 | 7.897 | 60.476 | 1.00 | 22.86 | Y | C |
| ATOM | 10379 | O | ILE | Y | 85 | 0.580 | 8.424 | 59.386 | 1.00 | 21.83 | Y | O |
| ATOM | 10380 | N | TYR | Y | 86 | 2.059 | 7.699 | 60.903 | 1.00 | 25.62 | Y | N |
| ATOM | 10381 | CA | TYR | Y | 86 | 3.203 | 8.005 | 60.049 | 1.00 | 21.59 | Y | C |
| ATOM | 10382 | CB | TYR | Y | 86 | 4.187 | 8.934 | 60.760 | 1.00 | 17.84 | Y | C |
| ATOM | 10383 | CG | TYR | Y | 86 | 3.629 | 10.322 | 60.952 | 1.00 | 22.38 | Y | C |
| ATOM | 10384 | CD1 | TYR | Y | 86 | 2.901 | 10.644 | 62.095 | 1.00 | 22.84 | Y | C |
| ATOM | 10385 | CE1 | TYR | Y | 86 | 2.363 | 11.913 | 62.272 | 1.00 | 20.34 | Y | C |
| ATOM | 10386 | CD2 | TYR | Y | 86 | 3.804 | 11.312 | 59.985 | 1.00 | 19.30 | Y | C |
| ATOM | 10387 | CE2 | TYR | Y | 86 | 3.265 | 12.589 | 60.156 | 1.00 | 20.22 | Y | C |
| ATOM | 10388 | CZ | TYR | Y | 86 | 2.547 | 12.878 | 61.307 | 1.00 | 23.03 | Y | C |
| ATOM | 10389 | OH | TYR | Y | 86 | 2.005 | 14.128 | 61.512 | 1.00 | 23.93 | Y | O |
| ATOM | 10390 | C | TYR | Y | 86 | 3.894 | 6.745 | 59.597 | 1.00 | 21.15 | Y | C |
| ATOM | 10391 | O | TYR | Y | 86 | 4.309 | 5.923 | 60.411 | 1.00 | 26.93 | Y | O |
| ATOM | 10392 | N | PHE | Y | 87 | 3.949 | 6.564 | 58.284 | 1.00 | 23.50 | Y | N |
| ATOM | 10393 | CA | PHE | Y | 87 | 4.594 | 5.396 | 57.702 | 1.00 | 19.47 | Y | C |
| ATOM | 10394 | CB | PHE | Y | 87 | 3.744 | 4.828 | 56.567 | 1.00 | 18.07 | Y | C |
| ATOM | 10395 | CG | PHE | Y | 87 | 2.498 | 4.133 | 57.028 | 1.00 | 19.78 | Y | C |
| ATOM | 10396 | CD1 | PHE | Y | 87 | 2.518 | 2.773 | 57.335 | 1.00 | 22.86 | Y | C |
| ATOM | 10397 | CD2 | PHE | Y | 87 | 1.303 | 4.829 | 57.146 | 1.00 | 16.97 | Y | C |
| ATOM | 10398 | CE1 | PHE | Y | 87 | 1.356 | 2.116 | 57.754 | 1.00 | 22.53 | Y | C |
| ATOM | 10399 | CE2 | PHE | Y | 87 | 0.140 | 4.182 | 57.563 | 1.00 | 22.59 | Y | C |
| ATOM | 10400 | CZ | PHE | Y | 87 | 0.166 | 2.823 | 57.867 | 1.00 | 15.76 | Y | C |
| ATOM | 10401 | C | PHE | Y | 87 | 5.957 | 5.774 | 57.157 | 1.00 | 22.97 | Y | C |
| ATOM | 10402 | O | PHE | Y | 87 | 6.149 | 6.871 | 56.612 | 1.00 | 23.31 | Y | O |
| ATOM | 10403 | N | CYS | Y | 88 | 6.905 | 4.877 | 57.375 | 1.00 | 22.80 | Y | N |
| ATOM | 10404 | CA | CYS | Y | 88 | 8.265 | 5.022 | 56.894 | 1.00 | 26.65 | Y | C |
| ATOM | 10405 | C | CYS | Y | 88 | 8.416 | 4.011 | 55.759 | 1.00 | 28.16 | Y | C |
| ATOM | 10406 | O | CYS | Y | 88 | 7.831 | 2.919 | 55.807 | 1.00 | 23.68 | Y | O |
| ATOM | 10407 | CB | CYS | Y | 88 | 9.271 | 4.662 | 57.992 | 1.00 | 29.53 | Y | C |
| ATOM | 10408 | SG | CYS | Y | 88 | 10.969 | 4.551 | 57.355 | 1.00 | 47.60 | Y | S |
| ATOM | 10409 | N | ALA | Y | 89 | 9.251 | 4.354 | 54.781 | 1.00 | 24.75 | Y | N |
| ATOM | 10410 | CA | ALA | Y | 89 | 9.510 | 3.489 | 53.636 | 1.00 | 26.55 | Y | C |
| ATOM | 10411 | CB | ALA | Y | 89 | 8.587 | 3.885 | 52.464 | 1.00 | 18.03 | Y | C |
| ATOM | 10412 | C | ALA | Y | 89 | 10.990 | 3.590 | 53.236 | 1.00 | 27.36 | Y | C |
| ATOM | 10413 | O | ALA | Y | 89 | 11.538 | 4.692 | 53.133 | 1.00 | 30.83 | Y | O |
| ATOM | 10414 | N | LEU | Y | 90 | 11.637 | 2.441 | 53.049 | 1.00 | 27.93 | Y | N |
| ATOM | 10415 | CA | LEU | Y | 90 | 13.046 | 2.383 | 52.667 | 1.00 | 27.57 | Y | C |
| ATOM | 10416 | CB | LEU | Y | 90 | 13.864 | 1.669 | 53.740 | 1.00 | 27.45 | Y | C |
| ATOM | 10417 | CG | LEU | Y | 90 | 13.742 | 2.222 | 55.160 | 1.00 | 30.91 | Y | C |
| ATOM | 10418 | CD1 | LEU | Y | 90 | 14.635 | 1.415 | 56.097 | 1.00 | 22.15 | Y | C |
| ATOM | 10419 | CD2 | LEU | Y | 90 | 14.121 | 3.697 | 55.194 | 1.00 | 27.82 | Y | C |
| ATOM | 10420 | C | LEU | Y | 90 | 13.207 | 1.634 | 51.346 | 1.00 | 31.60 | Y | C |
| ATOM | 10421 | O | LEU | Y | 90 | 12.558 | 0.617 | 51.113 | 1.00 | 34.27 | Y | O |
| ATOM | 10422 | N | TRP | Y | 91 | 14.094 | 2.144 | 50.498 | 1.00 | 31.05 | Y | N |
| ATOM | 10423 | CA | TRP | Y | 91 | 14.379 | 1.569 | 49.190 | 1.00 | 29.65 | Y | C |
| ATOM | 10424 | CB | TRP | Y | 91 | 14.618 | 2.707 | 48.190 | 1.00 | 28.00 | Y | C |
| ATOM | 10425 | CG | TRP | Y | 91 | 15.054 | 2.281 | 46.802 | 1.00 | 32.96 | Y | C |
| ATOM | 10426 | CD2 | TRP | Y | 91 | 14.219 | 1.778 | 45.749 | 1.00 | 27.74 | Y | C |
| ATOM | 10427 | CE2 | TRP | Y | 91 | 15.040 | 1.603 | 44.616 | 1.00 | 29.07 | Y | C |
| ATOM | 10428 | CE3 | TRP | Y | 91 | 12.860 | 1.465 | 45.654 | 1.00 | 27.76 | Y | C |
| ATOM | 10429 | CD1 | TRP | Y | 91 | 16.310 | 2.368 | 46.279 | 1.00 | 27.92 | Y | C |
| ATOM | 10430 | NE1 | TRP | Y | 91 | 16.310 | 1.964 | 44.973 | 1.00 | 27.19 | Y | N |
| ATOM | 10431 | CZ2 | TRP | Y | 91 | 14.546 | 1.134 | 43.394 | 1.00 | 28.77 | Y | C |
| ATOM | 10432 | CZ3 | TRP | Y | 91 | 12.364 | 0.995 | 44.441 | 1.00 | 28.76 | Y | C |
| ATOM | 10433 | CH2 | TRP | Y | 91 | 13.205 | 0.837 | 43.328 | 1.00 | 30.27 | Y | C |
| ATOM | 10434 | C | TRP | Y | 91 | 15.597 | 0.653 | 49.248 | 1.00 | 31.41 | Y | C |
| ATOM | 10435 | O | TRP | Y | 91 | 16.642 | 1.037 | 49.769 | 1.00 | 36.49 | Y | O |
| ATOM | 10436 | N | TYR | Y | 92 | 15.450 | −0.574 | 48.760 | 1.00 | 27.65 | Y | N |
| ATOM | 10437 | CA | TYR | Y | 92 | 16.566 | −1.505 | 48.754 | 1.00 | 28.52 | Y | C |
| ATOM | 10438 | CB | TYR | Y | 92 | 16.282 | −2.741 | 49.619 | 1.00 | 25.67 | Y | C |
| ATOM | 10439 | CG | TYR | Y | 92 | 16.011 | −2.447 | 51.078 | 1.00 | 30.08 | Y | C |
| ATOM | 10440 | CD1 | TYR | Y | 92 | 14.728 | −2.129 | 51.514 | 1.00 | 28.42 | Y | C |
| ATOM | 10441 | CE1 | TYR | Y | 92 | 14.460 | −1.888 | 52.853 | 1.00 | 30.09 | Y | C |
| ATOM | 10442 | CD2 | TYR | Y | 92 | 17.032 | −2.513 | 52.032 | 1.00 | 32.55 | Y | C |
| ATOM | 10443 | CE2 | TYR | Y | 92 | 16.774 | −2.275 | 53.383 | 1.00 | 24.58 | Y | C |
| ATOM | 10444 | CZ | TYR | Y | 92 | 15.483 | −1.967 | 53.785 | 1.00 | 31.73 | Y | C |
| ATOM | 10445 | OH | TYR | Y | 92 | 15.186 | −1.777 | 55.119 | 1.00 | 30.02 | Y | O |
| ATOM | 10446 | C | TYR | Y | 92 | 16.912 | −1.918 | 47.324 | 1.00 | 25.97 | Y | C |
| ATOM | 10447 | O | TYR | Y | 92 | 16.548 | −3.006 | 46.881 | 1.00 | 30.92 | Y | O |
| ATOM | 10448 | N | SER | Y | 93 | 17.579 | −1.012 | 46.605 | 1.00 | 26.20 | Y | N |
| ATOM | 10449 | CA | SER | Y | 93 | 18.034 | −1.231 | 45.219 | 1.00 | 27.82 | Y | C |
| ATOM | 10450 | CB | SER | Y | 93 | 18.893 | −2.499 | 45.122 | 1.00 | 24.81 | Y | C |
| ATOM | 10451 | OG | SER | Y | 93 | 19.948 | −2.493 | 46.062 | 1.00 | 29.69 | Y | O |
| ATOM | 10452 | C | SER | Y | 93 | 16.969 | −1.273 | 44.118 | 1.00 | 28.89 | Y | C |
| ATOM | 10453 | O | SER | Y | 93 | 17.048 | −0.516 | 43.153 | 1.00 | 26.35 | Y | O |
| ATOM | 10454 | N | ASN | Y | 94 | 15.974 | −2.147 | 44.281 | 1.00 | 29.43 | Y | N |
| ATOM | 10455 | CA | ASN | Y | 94 | 14.916 | −2.324 | 43.293 | 1.00 | 32.33 | Y | C |
| ATOM | 10456 | CB | ASN | Y | 94 | 15.186 | −3.586 | 42.475 | 1.00 | 29.17 | Y | C |
| ATOM | 10457 | CG | ASN | Y | 94 | 15.486 | −4.791 | 43.347 | 1.00 | 29.47 | Y | C |
| ATOM | 10458 | OD1 | ASN | Y | 94 | 14.848 | −5.017 | 44.369 | 1.00 | 32.84 | Y | O |
| ATOM | 10459 | ND2 | ASN | Y | 94 | 16.470 | −5.572 | 42.940 | 1.00 | 36.61 | Y | N |
| ATOM | 10460 | C | ASN | Y | 94 | 13.500 | −2.405 | 43.830 | 1.00 | 28.74 | Y | C |
| ATOM | 10461 | O | ASN | Y | 94 | 12.598 | −2.741 | 43.079 | 1.00 | 35.96 | Y | O |
| ATOM | 10462 | N | HIS | Y | 95 | 13.303 | −2.135 | 45.115 | 1.00 | 29.48 | Y | N |
| ATOM | 10463 | CA | HIS | Y | 95 | 11.971 | −2.203 | 45.704 | 1.00 | 25.16 | Y | C |
| ATOM | 10464 | CB | HIS | Y | 95 | 11.543 | −3.667 | 45.925 | 1.00 | 25.98 | Y | C |
| ATOM | 10465 | CG | HIS | Y | 95 | 12.329 | −4.400 | 46.977 | 1.00 | 25.99 | Y | C |
| ATOM | 10466 | CD2 | HIS | Y | 95 | 12.039 | −4.682 | 48.271 | 1.00 | 25.63 | Y | C |
| ATOM | 10467 | ND1 | HIS | Y | 95 | 13.552 | −4.988 | 46.726 | 1.00 | 26.54 | Y | N |
| ATOM | 10468 | CE1 | HIS | Y | 95 | 13.980 | −5.600 | 47.818 | 1.00 | 28.18 | Y | C |
| ATOM | 10469 | NE2 | HIS | Y | 95 | 13.081 | −5.429 | 48.770 | 1.00 | 26.28 | Y | N |
| ATOM | 10470 | C | HIS | Y | 95 | 11.841 | −1.430 | 47.011 | 1.00 | 29.20 | Y | C |
| ATOM | 10471 | O | HIS | Y | 95 | 12.838 | −1.114 | 47.678 | 1.00 | 22.68 | Y | O |
| ATOM | 10472 | N | TRP | Y | 96 | 10.599 | −1.117 | 47.362 | 1.00 | 25.24 | Y | N |
| ATOM | 10473 | CA | TRP | Y | 96 | 10.314 | −0.410 | 48.597 | 1.00 | 23.47 | Y | C |
| ATOM | 10474 | CB | TRP | Y | 96 | 9.251 | 0.650 | 48.356 | 1.00 | 20.43 | Y | C |
| ATOM | 10475 | CG | TRP | Y | 96 | 9.683 | 1.856 | 47.646 | 1.00 | 21.45 | Y | C |
| ATOM | 10476 | CD2 | TRP | Y | 96 | 10.397 | 2.969 | 48.203 | 1.00 | 20.36 | Y | C |
| ATOM | 10477 | CE2 | TRP | Y | 96 | 10.448 | 3.962 | 47.215 | 1.00 | 19.32 | Y | C |
| ATOM | 10478 | CE3 | TRP | Y | 96 | 10.985 | 3.223 | 49.447 | 1.00 | 24.09 | Y | C |
| ATOM | 10479 | CD1 | TRP | Y | 96 | 9.364 | 2.203 | 46.373 | 1.00 | 25.09 | Y | C |
| ATOM | 10480 | NE1 | TRP | Y | 96 | 9.813 | 3.469 | 46.104 | 1.00 | 26.67 | Y | N |
| ATOM | 10481 | CZ2 | TRP | Y | 96 | 11.060 | 5.193 | 47.427 | 1.00 | 17.98 | Y | C |
| ATOM | 10482 | CZ3 | TRP | Y | 96 | 11.596 | 4.445 | 49.658 | 1.00 | 20.80 | Y | C |
| ATOM | 10483 | CH2 | TRP | Y | 96 | 11.627 | 5.415 | 48.653 | 1.00 | 23.54 | Y | C |
| ATOM | 10484 | C | TRP | Y | 96 | 9.744 | −1.377 | 49.617 | 1.00 | 25.36 | Y | C |
| ATOM | 10485 | O | TRP | Y | 96 | 9.034 | −2.303 | 49.244 | 1.00 | 23.58 | Y | O |
| ATOM | 10486 | N | VAL | Y | 97 | 10.062 | −1.164 | 50.893 | 1.00 | 24.86 | Y | N |
| ATOM | 10487 | CA | VAL | Y | 97 | 9.502 | −1.966 | 51.986 | 1.00 | 25.54 | Y | C |
| ATOM | 10488 | CB | VAL | Y | 97 | 10.526 | −2.910 | 52.680 | 1.00 | 29.62 | Y | C |
| ATOM | 10489 | CG1 | VAL | Y | 97 | 9.877 | −3.585 | 53.911 | 1.00 | 21.60 | Y | C |
| ATOM | 10490 | CG2 | VAL | Y | 97 | 11.009 | −3.966 | 51.731 | 1.00 | 21.90 | Y | C |
| ATOM | 10491 | C | VAL | Y | 97 | 9.018 | −0.941 | 53.018 | 1.00 | 31.47 | Y | C |
| ATOM | 10492 | O | VAL | Y | 97 | 9.760 | −0.033 | 53.400 | 1.00 | 35.05 | Y | O |
| ATOM | 10493 | N | PHE | Y | 98 | 7.773 | −1.075 | 53.453 | 1.00 | 27.56 | Y | N |
| ATOM | 10494 | CA | PHE | Y | 98 | 7.207 | −0.157 | 54.427 | 1.00 | 25.18 | Y | C |
| ATOM | 10495 | CB | PHE | Y | 98 | 5.727 | 0.062 | 54.144 | 1.00 | 22.35 | Y | C |
| ATOM | 10496 | CG | PHE | Y | 98 | 5.454 | 0.935 | 52.965 | 1.00 | 20.92 | Y | C |
| ATOM | 10497 | CD1 | PHE | Y | 98 | 5.320 | 0.388 | 51.692 | 1.00 | 19.71 | Y | C |
| ATOM | 10498 | CD2 | PHE | Y | 98 | 5.261 | 2.301 | 53.135 | 1.00 | 16.93 | Y | C |
| ATOM | 10499 | CE1 | PHE | Y | 98 | 4.992 | 1.197 | 50.616 | 1.00 | 14.87 | Y | C |
| ATOM | 10500 | CE2 | PHE | Y | 98 | 4.933 | 3.112 | 52.069 | 1.00 | 12.62 | Y | C |
| ATOM | 10501 | CZ | PHE | Y | 98 | 4.797 | 2.561 | 50.808 | 1.00 | 16.80 | Y | C |
| ATOM | 10502 | C | PHE | Y | 98 | 7.332 | −0.631 | 55.870 | 1.00 | 31.57 | Y | C |
| ATOM | 10503 | O | PHE | Y | 98 | 7.389 | −1.836 | 56.149 | 1.00 | 29.14 | Y | O |
| ATOM | 10504 | N | GLY | Y | 99 | 7.384 | 0.328 | 56.789 | 1.00 | 25.40 | Y | N |
| ATOM | 10505 | CA | GLY | Y | 99 | 7.430 | −0.025 | 58.193 | 1.00 | 22.32 | Y | C |
| ATOM | 10506 | C | GLY | Y | 99 | 5.975 | −0.167 | 58.607 | 1.00 | 23.26 | Y | C |
| ATOM | 10507 | O | GLY | Y | 99 | 5.084 | 0.245 | 57.859 | 1.00 | 23.25 | Y | O |
| ATOM | 10508 | N | GLY | Y | 100 | 5.727 | −0.720 | 59.794 | 1.00 | 27.36 | Y | N |
| ATOM | 10509 | CA | GLY | Y | 100 | 4.364 | −0.900 | 60.274 | 1.00 | 20.12 | Y | C |
| ATOM | 10510 | C | GLY | Y | 100 | 3.581 | 0.370 | 60.604 | 1.00 | 21.61 | Y | C |
| ATOM | 10511 | O | GLY | Y | 100 | 2.391 | 0.310 | 60.891 | 1.00 | 29.77 | Y | O |
| ATOM | 10512 | N | GLY | Y | 101 | 4.219 | 1.529 | 60.524 | 1.00 | 20.45 | Y | N |
| ATOM | 10513 | CA | GLY | Y | 101 | 3.532 | 2.771 | 60.843 | 1.00 | 23.62 | Y | C |
| ATOM | 10514 | C | GLY | Y | 101 | 3.483 | 3.085 | 62.336 | 1.00 | 25.61 | Y | C |
| ATOM | 10515 | O | GLY | Y | 101 | 3.504 | 2.187 | 63.184 | 1.00 | 22.84 | Y | O |
| ATOM | 10516 | N | THR | Y | 102 | 3.422 | 4.371 | 62.653 | 1.00 | 24.54 | Y | N |
| ATOM | 10517 | CA | THR | Y | 102 | 3.381 | 4.822 | 64.033 | 1.00 | 27.33 | Y | C |
| ATOM | 10518 | CB | THR | Y | 102 | 4.616 | 5.692 | 64.388 | 1.00 | 29.55 | Y | C |
| ATOM | 10519 | OG1 | THR | Y | 102 | 5.807 | 4.900 | 64.286 | 1.00 | 29.60 | Y | O |
| ATOM | 10520 | CG2 | THR | Y | 102 | 4.485 | 6.250 | 65.821 | 1.00 | 23.32 | Y | C |
| ATOM | 10521 | C | THR | Y | 102 | 2.169 | 5.700 | 64.209 | 1.00 | 25.57 | Y | C |
| ATOM | 10522 | O | THR | Y | 102 | 2.020 | 6.680 | 63.490 | 1.00 | 28.07 | Y | O |
| ATOM | 10523 | N | LYS | Y | 103 | 1.292 | 5.351 | 65.143 | 1.00 | 22.98 | Y | N |
| ATOM | 10524 | CA | LYS | Y | 103 | 0.124 | 6.187 | 65.369 | 1.00 | 22.38 | Y | C |
| ATOM | 10525 | CB | LYS | Y | 103 | −1.069 | 5.385 | 65.927 | 1.00 | 26.69 | Y | C |
| ATOM | 10526 | CG | LYS | Y | 103 | −2.275 | 6.258 | 66.372 | 1.00 | 23.41 | Y | C |
| ATOM | 10527 | CD | LYS | Y | 103 | −3.417 | 5.367 | 66.894 | 1.00 | 33.32 | Y | C |
| ATOM | 10528 | CE | LYS | Y | 103 | −4.410 | 6.107 | 67.790 | 1.00 | 25.87 | Y | C |
| ATOM | 10529 | NZ | LYS | Y | 103 | −5.203 | 7.165 | 67.107 | 1.00 | 33.09 | Y | N |
| ATOM | 10530 | C | LYS | Y | 103 | 0.561 | 7.239 | 66.357 | 1.00 | 22.84 | Y | C |
| ATOM | 10531 | O | LYS | Y | 103 | 0.963 | 6.917 | 67.480 | 1.00 | 30.86 | Y | O |
| ATOM | 10532 | N | LEU | Y | 104 | 0.559 | 8.488 | 65.913 | 1.00 | 23.63 | Y | N |
| ATOM | 10533 | CA | LEU | Y | 104 | 0.953 | 9.582 | 66.772 | 1.00 | 24.24 | Y | C |
| ATOM | 10534 | CB | LEU | Y | 104 | 1.814 | 10.586 | 66.011 | 1.00 | 24.67 | Y | C |
| ATOM | 10535 | CG | LEU | Y | 104 | 1.990 | 11.910 | 66.780 | 1.00 | 26.70 | Y | C |
| ATOM | 10536 | CD1 | LEU | Y | 104 | 3.096 | 11.756 | 67.825 | 1.00 | 27.03 | Y | C |
| ATOM | 10537 | CD2 | LEU | Y | 104 | 2.299 | 13.059 | 65.838 | 1.00 | 20.23 | Y | C |
| ATOM | 10538 | C | LEU | Y | 104 | −0.262 | 10.319 | 67.298 | 1.00 | 26.53 | Y | C |
| ATOM | 10539 | O | LEU | Y | 104 | −1.070 | 10.816 | 66.515 | 1.00 | 22.46 | Y | O |
| ATOM | 10540 | N | THR | Y | 105 | −0.395 | 10.394 | 68.617 | 1.00 | 25.27 | Y | N |
| ATOM | 10541 | CA | THR | Y | 105 | −1.495 | 11.155 | 69.172 | 1.00 | 23.54 | Y | C |
| ATOM | 10542 | CB | THR | Y | 105 | −2.282 | 10.423 | 70.302 | 1.00 | 27.35 | Y | C |
| ATOM | 10543 | OG1 | THR | Y | 105 | −2.551 | 9.064 | 69.942 | 1.00 | 31.57 | Y | O |
| ATOM | 10544 | CG2 | THR | Y | 105 | −3.604 | 11.134 | 70.530 | 1.00 | 22.99 | Y | C |
| ATOM | 10545 | C | THR | Y | 105 | −0.983 | 12.484 | 69.741 | 1.00 | 24.30 | Y | C |
| ATOM | 10546 | O | THR | Y | 105 | 0.050 | 12.545 | 70.424 | 1.00 | 24.05 | Y | O |
| ATOM | 10547 | N | VAL | Y | 106 | −1.687 | 13.552 | 69.388 | 1.00 | 24.45 | Y | N |
| ATOM | 10548 | CA | VAL | Y | 106 | −1.390 | 14.885 | 69.890 | 1.00 | 25.78 | Y | C |
| ATOM | 10549 | CB | VAL | Y | 106 | −1.313 | 15.940 | 68.768 | 1.00 | 23.77 | Y | C |
| ATOM | 10550 | CG1 | VAL | Y | 106 | −1.172 | 17.317 | 69.379 | 1.00 | 24.95 | Y | C |
| ATOM | 10551 | CG2 | VAL | Y | 106 | −0.118 | 15.677 | 67.870 | 1.00 | 22.69 | Y | C |
| ATOM | 10552 | C | VAL | Y | 106 | −2.556 | 15.211 | 70.816 | 1.00 | 27.66 | Y | C |
| ATOM | 10553 | O | VAL | Y | 106 | −3.631 | 15.629 | 70.359 | 1.00 | 32.32 | Y | O |
| ATOM | 10554 | N | LEU | Y | 107 | −2.337 | 14.961 | 72.108 | 1.00 | 29.83 | Y | N |
| ATOM | 10555 | CA | LEU | Y | 107 | −3.309 | 15.170 | 73.187 | 1.00 | 29.24 | Y | C |
| ATOM | 10556 | CB | LEU | Y | 107 | −2.598 | 15.069 | 74.536 | 1.00 | 28.10 | Y | C |
| ATOM | 10557 | CG | LEU | Y | 107 | −2.346 | 13.656 | 75.066 | 1.00 | 32.22 | Y | C |
| ATOM | 10558 | CD1 | LEU | Y | 107 | −1.905 | 12.743 | 73.950 | 1.00 | 33.57 | Y | C |
| ATOM | 10559 | CD2 | LEU | Y | 107 | −1.299 | 13.689 | 76.186 | 1.00 | 28.29 | Y | C |
| ATOM | 10560 | C | LEU | Y | 107 | −4.163 | 16.433 | 73.146 | 1.00 | 26.55 | Y | C |
| ATOM | 10561 | O | LEU | Y | 107 | −3.663 | 17.540 | 73.324 | 1.00 | 31.45 | Y | O |
| ATOM | 10562 | N | GLY | Y | 108 | −5.462 | 16.246 | 72.930 | 1.00 | 31.32 | Y | N |
| ATOM | 10563 | CA | GLY | Y | 108 | −6.394 | 17.367 | 72.882 | 1.00 | 34.79 | Y | C |
| ATOM | 10564 | C | GLY | Y | 108 | −7.506 | 17.283 | 73.927 | 1.00 | 36.38 | Y | C |
| ATOM | 10565 | O | GLY | Y | 108 | −8.432 | 18.096 | 73.924 | 1.00 | 42.60 | Y | O |
| ATOM | 10566 | N | GLN | Y | 109 | −7.410 | 16.296 | 74.820 | 1.00 | 38.28 | Y | N |
| ATOM | 10567 | CA | GLN | Y | 109 | −8.389 | 16.069 | 75.890 | 1.00 | 31.60 | Y | C |
| ATOM | 10568 | CB | GLN | Y | 109 | −9.704 | 15.483 | 75.327 | 1.00 | 33.50 | Y | C |
| ATOM | 10569 | CG | GLN | Y | 109 | −9.572 | 14.344 | 74.303 | 1.00 | 35.84 | Y | C |
| ATOM | 10570 | CD | GLN | Y | 109 | −10.894 | 13.620 | 74.056 | 1.00 | 38.26 | Y | C |
| ATOM | 10571 | OE1 | GLN | Y | 109 | −11.521 | 13.136 | 74.993 | 1.00 | 40.35 | Y | O |
| ATOM | 10572 | NE2 | GLN | Y | 109 | −11.319 | 13.546 | 72.798 | 1.00 | 33.21 | Y | N |
| ATOM | 10573 | C | GLN | Y | 109 | −7.807 | 15.164 | 76.986 | 1.00 | 30.35 | Y | C |
| ATOM | 10574 | O | GLN | Y | 109 | −6.788 | 14.508 | 76.785 | 1.00 | 27.95 | Y | O |
| ATOM | 10575 | N | PRO | Y | 110 | −8.444 | 15.123 | 78.168 | 1.00 | 30.82 | Y | N |
| ATOM | 10576 | CD | PRO | Y | 110 | −9.647 | 15.845 | 78.618 | 1.00 | 30.55 | Y | C |
| ATOM | 10577 | CA | PRO | Y | 110 | −7.914 | 14.276 | 79.243 | 1.00 | 29.20 | Y | C |
| ATOM | 10578 | CB | PRO | Y | 110 | −8.908 | 14.501 | 80.392 | 1.00 | 28.57 | Y | C |
| ATOM | 10579 | CG | PRO | Y | 110 | −9.456 | 15.865 | 80.124 | 1.00 | 26.91 | Y | C |
| ATOM | 10580 | C | PRO | Y | 110 | −7.905 | 12.821 | 78.857 | 1.00 | 29.57 | Y | C |
| ATOM | 10581 | O | PRO | Y | 110 | −8.687 | 12.410 | 78.002 | 1.00 | 20.98 | Y | O |
| ATOM | 10582 | N | LYS | Y | 111 | −7.002 | 12.058 | 79.479 | 1.00 | 30.47 | Y | N |
| ATOM | 10583 | CA | LYS | Y | 111 | −6.900 | 10.621 | 79.252 | 1.00 | 27.39 | Y | C |
| ATOM | 10584 | CB | LYS | Y | 111 | −5.803 | 9.982 | 80.106 | 1.00 | 26.19 | Y | C |
| ATOM | 10585 | CG | LYS | Y | 111 | −4.412 | 10.514 | 79.923 | 1.00 | 39.17 | Y | C |
| ATOM | 10586 | CD | LYS | Y | 111 | −3.990 | 10.545 | 78.475 | 1.00 | 33.88 | Y | C |
| ATOM | 10587 | CE | LYS | Y | 111 | −2.511 | 10.817 | 78.399 | 1.00 | 38.12 | Y | C |
| ATOM | 10588 | NZ | LYS | Y | 111 | −1.781 | 9.805 | 79.204 | 1.00 | 33.17 | Y | N |
| ATOM | 10589 | C | LYS | Y | 111 | −8.221 | 10.007 | 79.704 | 1.00 | 30.61 | Y | C |
| ATOM | 10590 | O | LYS | Y | 111 | −8.899 | 10.538 | 80.590 | 1.00 | 28.97 | Y | O |
| ATOM | 10591 | N | SER | Y | 112 | −8.571 | 8.871 | 79.117 | 1.00 | 26.38 | Y | N |
| ATOM | 10592 | CA | SER | Y | 112 | −9.801 | 8.203 | 79.483 | 1.00 | 21.03 | Y | C |
| ATOM | 10593 | CB | SER | Y | 112 | −10.902 | 8.631 | 78.529 | 1.00 | 22.05 | Y | C |
| ATOM | 10594 | OG | SER | Y | 112 | −12.169 | 8.390 | 79.103 | 1.00 | 25.16 | Y | O |
| ATOM | 10595 | C | SER | Y | 112 | −9.628 | 6.694 | 79.435 | 1.00 | 24.69 | Y | C |
| ATOM | 10596 | O | SER | Y | 112 | −9.123 | 6.140 | 78.449 | 1.00 | 26.65 | Y | O |
| ATOM | 10597 | N | SER | Y | 113 | −9.965 | 6.033 | 80.537 | 1.00 | 22.27 | Y | N |
| ATOM | 10598 | CA | SER | Y | 113 | −9.883 | 4.578 | 80.581 | 1.00 | 20.72 | Y | C |
| ATOM | 10599 | CB | SER | Y | 113 | −9.802 | 4.062 | 82.016 | 1.00 | 24.11 | Y | C |
| ATOM | 10600 | OG | SER | Y | 113 | −8.495 | 4.240 | 82.554 | 1.00 | 27.76 | Y | O |
| ATOM | 10601 | C | SER | Y | 113 | −11.107 | 3.980 | 79.894 | 1.00 | 23.53 | Y | C |
| ATOM | 10602 | O | SER | Y | 113 | −12.190 | 4.573 | 79.885 | 1.00 | 19.11 | Y | O |
| ATOM | 10603 | N | PRO | Y | 114 | −10.939 | 2.794 | 79.312 | 1.00 | 22.19 | Y | N |
| ATOM | 10604 | CD | PRO | Y | 114 | −9.659 | 2.068 | 79.227 | 1.00 | 24.63 | Y | C |
| ATOM | 10605 | CA | PRO | Y | 114 | −11.999 | 2.083 | 78.605 | 1.00 | 27.13 | Y | C |
| ATOM | 10606 | CB | PRO | Y | 114 | −11.230 | 0.963 | 77.889 | 1.00 | 24.61 | Y | C |
| ATOM | 10607 | CG | PRO | Y | 114 | −10.091 | 0.689 | 78.798 | 1.00 | 25.67 | Y | C |
| ATOM | 10608 | C | PRO | Y | 114 | −13.124 | 1.511 | 79.468 | 1.00 | 26.46 | Y | C |
| ATOM | 10609 | O | PRO | Y | 114 | −12.859 | 0.982 | 80.553 | 1.00 | 24.34 | Y | O |
| ATOM | 10610 | N | SER | Y | 115 | −14.371 | 1.744 | 79.037 | 1.00 | 24.65 | Y | N |
| ATOM | 10611 | CA | SER | Y | 115 | −15.551 | 1.150 | 79.681 | 1.00 | 22.98 | Y | C |
| ATOM | 10612 | CB | SER | Y | 115 | −16.815 | 1.981 | 79.465 | 1.00 | 26.79 | Y | C |
| ATOM | 10613 | OG | SER | Y | 115 | −16.768 | 3.190 | 80.201 | 1.00 | 35.96 | Y | O |
| ATOM | 10614 | C | SER | Y | 115 | −15.677 | −0.189 | 78.923 | 1.00 | 23.07 | Y | C |
| ATOM | 10615 | O | SER | Y | 115 | −15.780 | −0.219 | 77.681 | 1.00 | 18.98 | Y | O |
| ATOM | 10616 | N | VAL | Y | 116 | −15.539 | −1.279 | 79.668 | 1.00 | 15.77 | Y | N |
| ATOM | 10617 | CA | VAL | Y | 116 | −15.584 | −2.641 | 79.140 | 1.00 | 19.55 | Y | C |
| ATOM | 10618 | CB | VAL | Y | 116 | −14.423 | −3.497 | 79.742 | 1.00 | 20.18 | Y | C |
| ATOM | 10619 | CG1 | VAL | Y | 116 | −14.273 | −4.824 | 78.975 | 1.00 | 22.86 | Y | C |
| ATOM | 10620 | CG2 | VAL | Y | 116 | −13.131 | −2.736 | 79.724 | 1.00 | 17.06 | Y | C |
| ATOM | 10621 | C | VAL | Y | 116 | −16.887 | −3.373 | 79.493 | 1.00 | 23.28 | Y | C |
| ATOM | 10622 | O | VAL | Y | 116 | −17.352 | −3.310 | 80.632 | 1.00 | 26.21 | Y | O |
| ATOM | 10623 | N | ASN | Y | 117 | −17.488 | −4.055 | 78.523 | 1.00 | 27.40 | Y | N |
| ATOM | 10624 | CA | ASN | Y | 117 | −18.714 | −4.823 | 78.782 | 1.00 | 24.85 | Y | C |
| ATOM | 10625 | CB | ASN | Y | 117 | −19.962 | −4.116 | 78.275 | 1.00 | 29.79 | Y | C |
| ATOM | 10626 | CG | ASN | Y | 117 | −19.971 | −2.658 | 78.621 | 1.00 | 43.43 | Y | C |
| ATOM | 10627 | OD1 | ASN | Y | 117 | −19.409 | −1.832 | 77.884 | 1.00 | 51.84 | Y | O |
| ATOM | 10628 | ND2 | ASN | Y | 117 | −20.566 | −2.319 | 79.768 | 1.00 | 50.69 | Y | N |
| ATOM | 10629 | C | ASN | Y | 117 | −18.555 | −6.143 | 78.059 | 1.00 | 27.58 | Y | C |
| ATOM | 10630 | O | ASN | Y | 117 | −18.245 | −6.169 | 76.866 | 1.00 | 26.33 | Y | O |
| ATOM | 10631 | N | LEU | Y | 118 | −18.706 | −7.234 | 78.802 | 1.00 | 19.81 | Y | N |
| ATOM | 10632 | CA | LEU | Y | 118 | −18.579 | −8.565 | 78.242 | 1.00 | 23.96 | Y | C |
| ATOM | 10633 | CB | LEU | Y | 118 | −17.652 | −9.436 | 79.095 | 1.00 | 17.06 | Y | C |
| ATOM | 10634 | CG | LEU | Y | 118 | −17.445 | −10.883 | 78.627 | 1.00 | 19.98 | Y | C |
| ATOM | 10635 | CD1 | LEU | Y | 118 | −16.948 | −10.876 | 77.224 | 1.00 | 22.69 | Y | C |
| ATOM | 10636 | CD2 | LEU | Y | 118 | −16.465 | −11.632 | 79.502 | 1.00 | 13.86 | Y | C |
| ATOM | 10637 | C | LEU | Y | 118 | −19.950 | −9.208 | 78.156 | 1.00 | 26.03 | Y | C |
| ATOM | 10638 | O | LEU | Y | 118 | −20.714 | −9.192 | 79.119 | 1.00 | 22.79 | Y | O |
| ATOM | 10639 | N | PHE | Y | 119 | −20.246 | −9.805 | 77.007 | 1.00 | 23.79 | Y | N |
| ATOM | 10640 | CA | PHE | Y | 119 | −21.545 | −10.441 | 76.807 | 1.00 | 23.98 | Y | C |
| ATOM | 10641 | CB | PHE | Y | 119 | −22.257 | −9.826 | 75.606 | 1.00 | 12.40 | Y | C |
| ATOM | 10642 | CG | PHE | Y | 119 | −22.775 | −8.443 | 75.857 | 1.00 | 12.61 | Y | C |
| ATOM | 10643 | CD1 | PHE | Y | 119 | −23.858 | −8.239 | 76.711 | 1.00 | 11.73 | Y | C |
| ATOM | 10644 | CD2 | PHE | Y | 119 | −22.205 | −7.348 | 75.226 | 1.00 | 10.94 | Y | C |
| ATOM | 10645 | CE1 | PHE | Y | 119 | −24.376 | −6.951 | 76.933 | 1.00 | 9.69 | Y | C |
| ATOM | 10646 | CE2 | PHE | Y | 119 | −22.710 | −6.053 | 75.441 | 1.00 | 12.74 | Y | C |
| ATOM | 10647 | CZ | PHE | Y | 119 | −23.804 | −5.858 | 76.299 | 1.00 | 13.35 | Y | C |
| ATOM | 10648 | C | PHE | Y | 119 | −21.465 | −11.940 | 76.633 | 1.00 | 18.31 | Y | C |
| ATOM | 10649 | O | PHE | Y | 119 | −20.555 | −12.452 | 75.996 | 1.00 | 27.39 | Y | O |
| ATOM | 10650 | N | PRO | Y | 120 | −22.350 | −12.675 | 77.299 | 1.00 | 20.36 | Y | N |
| ATOM | 10651 | CD | PRO | Y | 120 | −23.230 | −12.263 | 78.409 | 1.00 | 21.23 | Y | C |
| ATOM | 10652 | CA | PRO | Y | 120 | −22.327 | −14.128 | 77.153 | 1.00 | 19.31 | Y | C |
| ATOM | 10653 | CB | PRO | Y | 120 | −23.098 | −14.592 | 78.382 | 1.00 | 18.56 | Y | C |
| ATOM | 10654 | CG | PRO | Y | 120 | −24.077 | −13.484 | 78.604 | 1.00 | 19.49 | Y | C |
| ATOM | 10655 | C | PRO | Y | 120 | −23.058 | −14.518 | 75.857 | 1.00 | 21.37 | Y | C |
| ATOM | 10656 | O | PRO | Y | 120 | −23.755 | −13.694 | 75.265 | 1.00 | 26.49 | Y | O |
| ATOM | 10657 | N | PRO | Y | 121 | −22.837 | −15.750 | 75.358 | 1.00 | 22.99 | Y | N |
| ATOM | 10658 | CD | PRO | Y | 121 | −21.990 | −16.804 | 75.940 | 1.00 | 19.34 | Y | C |
| ATOM | 10659 | CA | PRO | Y | 121 | −23.499 | −16.219 | 74.129 | 1.00 | 24.33 | Y | C |
| ATOM | 10660 | CB | PRO | Y | 121 | −22.891 | −17.606 | 73.910 | 1.00 | 20.86 | Y | C |
| ATOM | 10661 | CG | PRO | Y | 121 | −21.643 | −17.614 | 74.744 | 1.00 | 25.41 | Y | C |
| ATOM | 10662 | C | PRO | Y | 121 | −24.994 | −16.367 | 74.436 | 1.00 | 20.94 | Y | C |
| ATOM | 10663 | O | PRO | Y | 121 | −25.359 | −16.778 | 75.539 | 1.00 | 29.14 | Y | O |
| ATOM | 10664 | N | SER | Y | 122 | −25.853 | −16.017 | 73.487 | 1.00 | 24.67 | Y | N |
| ATOM | 10665 | CA | SER | Y | 122 | −27.310 | −16.137 | 73.679 | 1.00 | 26.82 | Y | C |
| ATOM | 10666 | CB | SER | Y | 122 | −28.047 | −15.267 | 72.664 | 1.00 | 20.51 | Y | C |
| ATOM | 10667 | OG | SER | Y | 122 | −27.994 | −15.889 | 71.405 | 1.00 | 25.78 | Y | O |
| ATOM | 10668 | C | SER | Y | 122 | −27.787 | −17.588 | 73.511 | 1.00 | 17.03 | Y | C |
| ATOM | 10669 | O | SER | Y | 122 | −27.154 | −18.382 | 72.809 | 1.00 | 19.46 | Y | O |
| ATOM | 10670 | N | SER | Y | 123 | −28.910 | −17.928 | 74.134 | 1.00 | 17.70 | Y | N |
| ATOM | 10671 | CA | SER | Y | 123 | −29.448 | −19.288 | 74.004 | 1.00 | 25.52 | Y | C |
| ATOM | 10672 | CB | SER | Y | 123 | −30.712 | −19.487 | 74.842 | 1.00 | 24.83 | Y | C |
| ATOM | 10673 | OG | SER | Y | 123 | −31.786 | −18.731 | 74.308 | 1.00 | 41.57 | Y | O |
| ATOM | 10674 | C | SER | Y | 123 | −29.779 | −19.541 | 72.546 | 1.00 | 22.75 | Y | C |
| ATOM | 10675 | O | SER | Y | 123 | −29.562 | −20.637 | 72.043 | 1.00 | 26.39 | Y | O |
| ATOM | 10676 | N | GLU | Y | 124 | −30.247 | −18.497 | 71.861 | 1.00 | 17.20 | Y | N |
| ATOM | 10677 | CA | GLU | Y | 124 | −30.585 | −18.609 | 70.457 | 1.00 | 19.95 | Y | C |
| ATOM | 10678 | CB | GLU | Y | 124 | −31.095 | −17.277 | 69.916 | 1.00 | 23.08 | Y | C |
| ATOM | 10679 | CG | GLU | Y | 124 | −32.471 | −16.834 | 70.421 | 1.00 | 32.76 | Y | C |
| ATOM | 10680 | CD | GLU | Y | 124 | −32.410 | −15.993 | 71.703 | 1.00 | 36.44 | Y | C |
| ATOM | 10681 | OE1 | GLU | Y | 124 | −31.487 | −16.202 | 72.522 | 1.00 | 43.57 | Y | O |
| ATOM | 10682 | OE2 | GLU | Y | 124 | −33.291 | −15.121 | 71.894 | 1.00 | 36.12 | Y | O |
| ATOM | 10683 | C | GLU | Y | 124 | −29.388 | −19.090 | 69.634 | 1.00 | 26.77 | Y | C |
| ATOM | 10684 | O | GLU | Y | 124 | −29.475 | −20.092 | 68.915 | 1.00 | 28.41 | Y | O |
| ATOM | 10685 | N | GLU | Y | 125 | −28.254 | −18.405 | 69.777 | 1.00 | 29.19 | Y | N |
| ATOM | 10686 | CA | GLU | Y | 125 | −27.064 | −18.776 | 69.030 | 1.00 | 31.89 | Y | C |
| ATOM | 10687 | CB | GLU | Y | 125 | −25.970 | −17.726 | 69.180 | 1.00 | 34.81 | Y | C |
| ATOM | 10688 | CG | GLU | Y | 125 | −24.756 | −18.023 | 68.319 | 1.00 | 37.44 | Y | C |
| ATOM | 10689 | CD | GLU | Y | 125 | −23.523 | −17.263 | 68.746 | 1.00 | 37.00 | Y | C |
| ATOM | 10690 | OE1 | GLU | Y | 125 | −23.557 | −16.585 | 69.800 | 1.00 | 40.97 | Y | O |
| ATOM | 10691 | OE2 | GLU | Y | 125 | −22.511 | −17.353 | 68.022 | 1.00 | 35.42 | Y | O |
| ATOM | 10692 | C | GLU | Y | 125 | −26.540 | −20.158 | 69.411 | 1.00 | 30.77 | Y | C |
| ATOM | 10693 | O | GLU | Y | 125 | −26.055 | −20.886 | 68.551 | 1.00 | 28.14 | Y | O |
| ATOM | 10694 | N | LEU | Y | 126 | −26.654 | −20.528 | 70.685 | 1.00 | 28.34 | Y | N |
| ATOM | 10695 | CA | LEU | Y | 126 | −26.188 | −21.841 | 71.109 | 1.00 | 33.37 | Y | C |
| ATOM | 10696 | CB | LEU | Y | 126 | −26.343 | −22.018 | 72.616 | 1.00 | 32.60 | Y | C |
| ATOM | 10697 | CG | LEU | Y | 126 | −25.386 | −21.121 | 73.400 | 1.00 | 36.60 | Y | C |
| ATOM | 10698 | CD1 | LEU | Y | 126 | −25.623 | −21.262 | 74.911 | 1.00 | 31.00 | Y | C |
| ATOM | 10699 | CD2 | LEU | Y | 126 | −23.938 | −21.440 | 73.014 | 1.00 | 31.65 | Y | C |
| ATOM | 10700 | C | LEU | Y | 126 | −26.924 | −22.937 | 70.358 | 1.00 | 30.44 | Y | C |
| ATOM | 10701 | O | LEU | Y | 126 | −26.370 | −23.992 | 70.091 | 1.00 | 34.02 | Y | O |
| ATOM | 10702 | N | LYS | Y | 127 | −28.156 | −22.657 | 69.955 | 1.00 | 34.53 | Y | N |
| ATOM | 10703 | CA | LYS | Y | 127 | −28.928 | −23.635 | 69.209 | 1.00 | 34.98 | Y | C |
| ATOM | 10704 | CB | LYS | Y | 127 | −30.334 | −23.122 | 68.945 | 1.00 | 34.57 | Y | C |
| ATOM | 10705 | CG | LYS | Y | 127 | −31.229 | −23.097 | 70.172 | 1.00 | 38.71 | Y | C |
| ATOM | 10706 | CD | LYS | Y | 127 | −32.380 | −22.131 | 69.945 | 1.00 | 45.25 | Y | C |
| ATOM | 10707 | CE | LYS | Y | 127 | −33.419 | −22.252 | 71.024 | 1.00 | 52.64 | Y | C |
| ATOM | 10708 | NZ | LYS | Y | 127 | −34.135 | −23.557 | 70.887 | 1.00 | 58.95 | Y | N |
| ATOM | 10709 | C | LYS | Y | 127 | −28.226 | −23.990 | 67.898 | 1.00 | 33.22 | Y | C |
| ATOM | 10710 | O | LYS | Y | 127 | −28.398 | −25.095 | 67.391 | 1.00 | 37.32 | Y | O |
| ATOM | 10711 | N | THR | Y | 128 | −27.419 | −23.070 | 67.367 | 1.00 | 31.99 | Y | N |
| ATOM | 10712 | CA | THR | Y | 128 | −26.690 | −23.318 | 66.121 | 1.00 | 22.77 | Y | C |
| ATOM | 10713 | CB | THR | Y | 128 | −26.279 | −22.023 | 65.391 | 1.00 | 28.01 | Y | C |
| ATOM | 10714 | OG1 | THR | Y | 128 | −25.327 | −21.295 | 66.186 | 1.00 | 25.79 | Y | O |
| ATOM | 10715 | CG2 | THR | Y | 128 | −27.492 | −21.145 | 65.075 | 1.00 | 19.98 | Y | C |
| ATOM | 10716 | C | THR | Y | 128 | −25.419 | −24.085 | 66.408 | 1.00 | 30.90 | Y | C |
| ATOM | 10717 | O | THR | Y | 128 | −24.620 | −24.305 | 65.503 | 1.00 | 36.40 | Y | O |
| ATOM | 10718 | N | LYS | Y | 129 | −25.220 | −24.449 | 67.677 | 1.00 | 35.07 | Y | N |
| ATOM | 10719 | CA | LYS | Y | 129 | −24.053 | −25.208 | 68.123 | 1.00 | 36.28 | Y | C |
| ATOM | 10720 | CB | LYS | Y | 129 | −24.022 | −26.568 | 67.414 | 1.00 | 37.51 | Y | C |
| ATOM | 10721 | CG | LYS | Y | 129 | −25.367 | −27.310 | 67.546 | 1.00 | 38.85 | Y | C |
| ATOM | 10722 | CD | LYS | Y | 129 | −25.373 | −28.650 | 66.834 | 1.00 | 47.86 | Y | C |
| ATOM | 10723 | CE | LYS | Y | 129 | −24.501 | −29.678 | 67.573 | 1.00 | 56.92 | Y | C |
| ATOM | 10724 | NZ | LYS | Y | 129 | −24.390 | −30.994 | 66.868 | 1.00 | 51.11 | Y | N |
| ATOM | 10725 | C | LYS | Y | 129 | −22.763 | −24.403 | 67.939 | 1.00 | 42.32 | Y | C |
| ATOM | 10726 | O | LYS | Y | 129 | −21.685 | −24.950 | 67.661 | 1.00 | 49.52 | Y | O |
| ATOM | 10727 | N | LYS | Y | 130 | −22.908 | −23.089 | 68.129 | 1.00 | 42.67 | Y | N |
| ATOM | 10728 | CA | LYS | Y | 130 | −21.831 | −22.101 | 68.024 | 1.00 | 40.61 | Y | C |
| ATOM | 10729 | CB | LYS | Y | 130 | −21.957 | −21.328 | 66.699 | 1.00 | 42.03 | Y | C |
| ATOM | 10730 | CG | LYS | Y | 130 | −21.663 | −22.154 | 65.447 | 1.00 | 45.28 | Y | C |
| ATOM | 10731 | CD | LYS | Y | 130 | −20.198 | −22.613 | 65.438 | 1.00 | 50.16 | Y | C |
| ATOM | 10732 | CE | LYS | Y | 130 | −19.822 | −23.383 | 64.169 | 1.00 | 51.30 | Y | C |
| ATOM | 10733 | NZ | LYS | Y | 130 | −18.398 | −23.855 | 64.233 | 1.00 | 50.37 | Y | N |
| ATOM | 10734 | C | LYS | Y | 130 | −21.970 | −21.118 | 69.194 | 1.00 | 37.20 | Y | C |
| ATOM | 10735 | O | LYS | Y | 130 | −23.066 | −20.922 | 69.720 | 1.00 | 35.77 | Y | O |
| ATOM | 10736 | N | ALA | Y | 131 | −20.867 | −20.521 | 69.629 | 1.00 | 35.04 | Y | N |
| ATOM | 10737 | CA | ALA | Y | 131 | −20.945 | −19.560 | 70.730 | 1.00 | 32.42 | Y | C |
| ATOM | 10738 | CB | ALA | Y | 131 | −20.671 | −20.236 | 72.055 | 1.00 | 30.66 | Y | C |
| ATOM | 10739 | C | ALA | Y | 131 | −20.006 | −18.381 | 70.524 | 1.00 | 33.32 | Y | C |
| ATOM | 10740 | O | ALA | Y | 131 | −18.839 | −18.550 | 70.178 | 1.00 | 37.47 | Y | O |
| ATOM | 10741 | N | THR | Y | 132 | −20.535 | −17.181 | 70.700 | 1.00 | 28.96 | Y | N |
| ATOM | 10742 | CA | THR | Y | 132 | −19.728 | −15.990 | 70.537 | 1.00 | 34.74 | Y | C |
| ATOM | 10743 | CB | THR | Y | 132 | −20.189 | −15.185 | 69.321 | 1.00 | 33.18 | Y | C |
| ATOM | 10744 | OG1 | THR | Y | 132 | −20.071 | −15.999 | 68.152 | 1.00 | 38.53 | Y | O |
| ATOM | 10745 | CG2 | THR | Y | 132 | −19.351 | −13.935 | 69.158 | 1.00 | 32.23 | Y | C |
| ATOM | 10746 | C | THR | Y | 132 | −19.805 | −15.094 | 71.764 | 1.00 | 32.66 | Y | C |
| ATOM | 10747 | O | THR | Y | 132 | −20.890 | −14.768 | 72.234 | 1.00 | 37.74 | Y | O |
| ATOM | 10748 | N | LEU | Y | 133 | −18.649 | −14.744 | 72.311 | 1.00 | 33.40 | Y | N |
| ATOM | 10749 | CA | LEU | Y | 133 | −18.611 | −13.840 | 73.448 | 1.00 | 30.81 | Y | C |
| ATOM | 10750 | CB | LEU | Y | 133 | −17.606 | −14.293 | 74.508 | 1.00 | 32.22 | Y | C |
| ATOM | 10751 | CG | LEU | Y | 133 | −18.035 | −15.511 | 75.328 | 1.00 | 23.81 | Y | C |
| ATOM | 10752 | CD1 | LEU | Y | 133 | −17.490 | −16.750 | 74.703 | 1.00 | 30.08 | Y | C |
| ATOM | 10753 | CD2 | LEU | Y | 133 | −17.508 | −15.392 | 76.741 | 1.00 | 32.54 | Y | C |
| ATOM | 10754 | C | LEU | Y | 133 | −18.237 | −12.491 | 72.864 | 1.00 | 29.82 | Y | C |
| ATOM | 10755 | O | LEU | Y | 133 | −17.341 | −12.406 | 72.022 | 1.00 | 28.69 | Y | O |
| ATOM | 10756 | N | VAL | Y | 134 | −19.011 | −11.469 | 73.219 | 1.00 | 22.95 | Y | N |
| ATOM | 10757 | CA | VAL | Y | 134 | −18.780 | −10.125 | 72.727 | 1.00 | 16.30 | Y | C |
| ATOM | 10758 | CB | VAL | Y | 134 | −20.054 | −9.509 | 72.149 | 1.00 | 15.70 | Y | C |
| ATOM | 10759 | CG1 | VAL | Y | 134 | −19.753 | −8.111 | 71.593 | 1.00 | 13.84 | Y | C |
| ATOM | 10760 | CG2 | VAL | Y | 134 | −20.631 | −10.419 | 71.047 | 1.00 | 15.12 | Y | C |
| ATOM | 10761 | C | VAL | Y | 134 | −18.256 | −9.201 | 73.818 | 1.00 | 23.40 | Y | C |
| ATOM | 10762 | O | VAL | Y | 134 | −18.858 | −9.061 | 74.879 | 1.00 | 23.75 | Y | O |
| ATOM | 10763 | N | CYS | Y | 135 | −17.108 | −8.590 | 73.552 | 1.00 | 24.59 | Y | N |
| ATOM | 10764 | CA | CYS | Y | 135 | −16.496 | −7.658 | 74.485 | 1.00 | 20.82 | Y | C |
| ATOM | 10765 | C | CYS | Y | 135 | −16.513 | −6.256 | 73.875 | 1.00 | 19.24 | Y | C |
| ATOM | 10766 | O | CYS | Y | 135 | −15.825 | −6.008 | 72.893 | 1.00 | 25.03 | Y | O |
| ATOM | 10767 | CB | CYS | Y | 135 | −15.059 | −8.085 | 74.754 | 1.00 | 22.99 | Y | C |
| ATOM | 10768 | SG | CYS | Y | 135 | −14.195 | −7.099 | 76.012 | 1.00 | 24.81 | Y | S |
| ATOM | 10769 | N | THR | Y | 136 | −17.360 | −5.371 | 74.389 | 1.00 | 15.36 | Y | N |
| ATOM | 10770 | CA | THR | Y | 136 | −17.417 | −4.010 | 73.867 | 1.00 | 17.19 | Y | C |
| ATOM | 10771 | CB | THR | Y | 136 | −18.884 | −3.473 | 73.733 | 1.00 | 21.55 | Y | C |
| ATOM | 10772 | OG1 | THR | Y | 136 | −19.518 | −3.392 | 75.012 | 1.00 | 22.10 | Y | O |
| ATOM | 10773 | CG2 | THR | Y | 136 | −19.708 | −4.392 | 72.828 | 1.00 | 21.59 | Y | C |
| ATOM | 10774 | C | THR | Y | 136 | −16.505 | −3.095 | 74.711 | 1.00 | 22.45 | Y | C |
| ATOM | 10775 | O | THR | Y | 136 | −16.438 | −3.204 | 75.937 | 1.00 | 25.91 | Y | O |
| ATOM | 10776 | N | ILE | Y | 137 | −15.757 | −2.236 | 74.028 | 1.00 | 22.94 | Y | N |
| ATOM | 10777 | CA | ILE | Y | 137 | −14.780 | −1.355 | 74.665 | 1.00 | 22.46 | Y | C |
| ATOM | 10778 | CB | ILE | Y | 137 | −13.349 | −1.795 | 74.266 | 1.00 | 20.17 | Y | C |
| ATOM | 10779 | CG2 | ILE | Y | 137 | −12.312 | −1.028 | 75.062 | 1.00 | 22.97 | Y | C |
| ATOM | 10780 | CG1 | ILE | Y | 137 | −13.196 | −3.308 | 74.470 | 1.00 | 21.23 | Y | C |
| ATOM | 10781 | CD1 | ILE | Y | 137 | −12.079 | −3.964 | 73.700 | 1.00 | 12.18 | Y | C |
| ATOM | 10782 | C | ILE | Y | 137 | −15.030 | 0.069 | 74.180 | 1.00 | 24.99 | Y | C |
| ATOM | 10783 | O | ILE | Y | 137 | −14.816 | 0.385 | 73.006 | 1.00 | 23.32 | Y | O |
| ATOM | 10784 | N | THR | Y | 138 | −15.485 | 0.934 | 75.079 | 1.00 | 21.73 | Y | N |
| ATOM | 10785 | CA | THR | Y | 138 | −15.779 | 2.292 | 74.668 | 1.00 | 18.99 | Y | C |
| ATOM | 10786 | CB | THR | Y | 138 | −17.318 | 2.522 | 74.600 | 1.00 | 19.19 | Y | C |
| ATOM | 10787 | OG1 | THR | Y | 138 | −17.861 | 2.537 | 75.924 | 1.00 | 24.65 | Y | O |
| ATOM | 10788 | CG2 | THR | Y | 138 | −18.015 | 1.400 | 73.817 | 1.00 | 10.27 | Y | C |
| ATOM | 10789 | C | THR | Y | 138 | −15.140 | 3.367 | 75.550 | 1.00 | 28.75 | Y | C |
| ATOM | 10790 | O | THR | Y | 138 | −14.566 | 3.087 | 76.609 | 1.00 | 30.65 | Y | O |
| ATOM | 10791 | N | GLU | Y | 139 | −15.222 | 4.598 | 75.074 | 1.00 | 23.53 | Y | N |
| ATOM | 10792 | CA | GLU | Y | 139 | −14.726 | 5.759 | 75.788 | 1.00 | 28.59 | Y | C |
| ATOM | 10793 | CB | GLU | Y | 139 | −15.728 | 6.156 | 76.878 | 1.00 | 24.87 | Y | C |
| ATOM | 10794 | CG | GLU | Y | 139 | −17.073 | 6.632 | 76.316 | 1.00 | 20.40 | Y | C |
| ATOM | 10795 | CD | GLU | Y | 139 | −17.669 | 7.788 | 77.099 | 0.00 | 22.78 | Y | C |
| ATOM | 10796 | OE1 | GLU | Y | 139 | −17.125 | 8.910 | 77.015 | 0.00 | 22.78 | Y | O |
| ATOM | 10797 | OE2 | GLU | Y | 139 | −18.690 | 7.579 | 77.787 | 0.00 | 22.51 | Y | O |
| ATOM | 10798 | C | GLU | Y | 139 | −13.279 | 5.738 | 76.316 | 1.00 | 33.13 | Y | C |
| ATOM | 10799 | O | GLU | Y | 139 | −13.021 | 6.076 | 77.477 | 1.00 | 34.51 | Y | O |
| ATOM | 10800 | N | PHE | Y | 140 | −12.335 | 5.375 | 75.449 | 1.00 | 28.49 | Y | N |
| ATOM | 10801 | CA | PHE | Y | 140 | −10.935 | 5.356 | 75.834 | 1.00 | 28.55 | Y | C |
| ATOM | 10802 | CB | PHE | Y | 140 | −10.309 | 3.954 | 75.803 | 1.00 | 27.31 | Y | C |
| ATOM | 10803 | CG | PHE | Y | 140 | −10.338 | 3.275 | 74.464 | 1.00 | 27.65 | Y | C |
| ATOM | 10804 | CD1 | PHE | Y | 140 | −9.289 | 3.421 | 73.571 | 1.00 | 30.73 | Y | C |
| ATOM | 10805 | CD2 | PHE | Y | 140 | −11.380 | 2.418 | 74.129 | 1.00 | 31.50 | Y | C |
| ATOM | 10806 | CE1 | PHE | Y | 140 | −9.271 | 2.724 | 72.372 | 1.00 | 31.06 | Y | C |
| ATOM | 10807 | CE2 | PHE | Y | 140 | −11.371 | 1.714 | 72.924 | 1.00 | 32.26 | Y | C |
| ATOM | 10808 | CZ | PHE | Y | 140 | −10.311 | 1.871 | 72.048 | 1.00 | 32.72 | Y | C |
| ATOM | 10809 | C | PHE | Y | 140 | −10.118 | 6.333 | 75.023 | 1.00 | 34.01 | Y | C |
| ATOM | 10810 | O | PHE | Y | 140 | −10.338 | 6.517 | 73.821 | 1.00 | 28.83 | Y | O |
| ATOM | 10811 | N | TYR | Y | 141 | −9.189 | 6.983 | 75.714 | 1.00 | 33.83 | Y | N |
| ATOM | 10812 | CA | TYR | Y | 141 | −8.316 | 7.969 | 75.100 | 1.00 | 34.80 | Y | C |
| ATOM | 10813 | CB | TYR | Y | 141 | −8.997 | 9.331 | 75.100 | 1.00 | 27.68 | Y | C |
| ATOM | 10814 | CG | TYR | Y | 141 | −8.173 | 10.373 | 74.419 | 1.00 | 31.56 | Y | C |
| ATOM | 10815 | CD1 | TYR | Y | 141 | −8.155 | 10.465 | 73.025 | 1.00 | 30.78 | Y | C |
| ATOM | 10816 | CE1 | TYR | Y | 141 | −7.386 | 11.426 | 72.381 | 1.00 | 33.76 | Y | C |
| ATOM | 10817 | CD2 | TYR | Y | 141 | −7.394 | 11.269 | 75.158 | 1.00 | 28.64 | Y | C |
| ATOM | 10818 | CE2 | TYR | Y | 141 | −6.621 | 12.233 | 74.523 | 1.00 | 28.44 | Y | C |
| ATOM | 10819 | CZ | TYR | Y | 141 | −6.625 | 12.306 | 73.136 | 1.00 | 30.02 | Y | C |
| ATOM | 10820 | OH | TYR | Y | 141 | −5.893 | 13.265 | 72.493 | 1.00 | 36.27 | Y | O |
| ATOM | 10821 | C | TYR | Y | 141 | −6.949 | 8.062 | 75.798 | 1.00 | 33.25 | Y | C |
| ATOM | 10822 | O | TYR | Y | 141 | −6.856 | 8.141 | 77.023 | 1.00 | 33.69 | Y | O |
| ATOM | 10823 | N | PRO | Y | 142 | −5.869 | 8.040 | 75.020 | 1.00 | 36.99 | Y | N |
| ATOM | 10824 | CD | PRO | Y | 142 | −4.523 | 8.213 | 75.586 | 1.00 | 36.52 | Y | C |
| ATOM | 10825 | CA | PRO | Y | 142 | −5.804 | 7.927 | 73.558 | 1.00 | 36.61 | Y | C |
| ATOM | 10826 | CB | PRO | Y | 142 | −4.314 | 8.119 | 73.277 | 1.00 | 37.12 | Y | C |
| ATOM | 10827 | CG | PRO | Y | 142 | −3.663 | 7.606 | 74.520 | 1.00 | 40.32 | Y | C |
| ATOM | 10828 | C | PRO | Y | 142 | −6.332 | 6.605 | 72.990 | 1.00 | 34.30 | Y | C |
| ATOM | 10829 | O | PRO | Y | 142 | −6.478 | 5.616 | 73.713 | 1.00 | 29.74 | Y | O |
| ATOM | 10830 | N | GLY | Y | 143 | −6.603 | 6.599 | 71.685 | 1.00 | 32.98 | Y | N |
| ATOM | 10831 | CA | GLY | Y | 143 | −7.137 | 5.419 | 71.019 | 1.00 | 27.86 | Y | C |
| ATOM | 10832 | C | GLY | Y | 143 | −6.201 | 4.261 | 70.728 | 1.00 | 30.63 | Y | C |
| ATOM | 10833 | O | GLY | Y | 143 | −5.969 | 3.911 | 69.570 | 1.00 | 34.77 | Y | O |
| ATOM | 10834 | N | ALA | Y | 144 | −5.705 | 3.633 | 71.784 | 1.00 | 25.41 | Y | N |
| ATOM | 10835 | CA | ALA | Y | 144 | −4.800 | 2.517 | 71.655 | 1.00 | 26.75 | Y | C |
| ATOM | 10836 | CB | ALA | Y | 144 | −3.328 | 3.025 | 71.633 | 1.00 | 30.23 | Y | C |
| ATOM | 10837 | C | ALA | Y | 144 | −5.020 | 1.583 | 72.832 | 1.00 | 25.94 | Y | C |
| ATOM | 10838 | O | ALA | Y | 144 | −4.980 | 2.019 | 73.981 | 1.00 | 27.37 | Y | O |
| ATOM | 10839 | N | VAL | Y | 145 | −5.259 | 0.305 | 72.537 | 1.00 | 24.26 | Y | N |
| ATOM | 10840 | CA | VAL | Y | 145 | −5.468 | −0.719 | 73.556 | 1.00 | 20.91 | Y | C |
| ATOM | 10841 | CB | VAL | Y | 145 | −6.977 | −0.896 | 73.953 | 1.00 | 28.48 | Y | C |
| ATOM | 10842 | CG1 | VAL | Y | 145 | −7.474 | 0.280 | 74.755 | 1.00 | 31.95 | Y | C |
| ATOM | 10843 | CG2 | VAL | Y | 145 | −7.854 | −1.094 | 72.694 | 1.00 | 24.54 | Y | C |
| ATOM | 10844 | C | VAL | Y | 145 | −5.045 | −2.059 | 73.003 | 1.00 | 21.04 | Y | C |
| ATOM | 10845 | O | VAL | Y | 145 | −4.987 | −2.264 | 71.794 | 1.00 | 27.14 | Y | O |
| ATOM | 10846 | N | ARG | Y | 146 | −4.851 | −2.997 | 73.910 | 1.00 | 21.74 | Y | N |
| ATOM | 10847 | CA | ARG | Y | 146 | −4.490 | −4.361 | 73.571 | 1.00 | 31.95 | Y | C |
| ATOM | 10848 | CB | ARG | Y | 146 | −3.053 | −4.637 | 74.034 | 1.00 | 34.09 | Y | C |
| ATOM | 10849 | CG | ARG | Y | 146 | −2.077 | −3.562 | 73.538 | 1.00 | 44.22 | Y | C |
| ATOM | 10850 | CD | ARG | Y | 146 | −1.915 | −3.590 | 72.002 | 1.00 | 50.07 | Y | C |
| ATOM | 10851 | NE | ARG | Y | 146 | −0.820 | −4.479 | 71.594 | 1.00 | 61.66 | Y | N |
| ATOM | 10852 | CZ | ARG | Y | 146 | −0.917 | −5.488 | 70.724 | 1.00 | 65.47 | Y | C |
| ATOM | 10853 | NH1 | ARG | Y | 146 | −2.074 | −5.771 | 70.129 | 1.00 | 62.59 | Y | N |
| ATOM | 10854 | NH2 | ARG | Y | 146 | 0.149 | −6.249 | 70.479 | 1.00 | 66.80 | Y | N |
| ATOM | 10855 | C | ARG | Y | 146 | −5.529 | −5.242 | 74.291 | 1.00 | 26.18 | Y | C |
| ATOM | 10856 | O | ARG | Y | 146 | −5.861 | −4.990 | 75.443 | 1.00 | 28.52 | Y | O |
| ATOM | 10857 | N | VAL | Y | 147 | −6.104 | −6.213 | 73.584 | 1.00 | 28.59 | Y | N |
| ATOM | 10858 | CA | VAL | Y | 147 | −7.129 | −7.081 | 74.164 | 1.00 | 20.82 | Y | C |
| ATOM | 10859 | CB | VAL | Y | 147 | −8.426 | −7.054 | 73.291 | 1.00 | 24.73 | Y | C |
| ATOM | 10860 | CG1 | VAL | Y | 147 | −9.440 | −8.066 | 73.784 | 1.00 | 27.92 | Y | C |
| ATOM | 10861 | CG2 | VAL | Y | 147 | −9.043 | −5.678 | 73.318 | 1.00 | 22.03 | Y | C |
| ATOM | 10862 | C | VAL | Y | 147 | −6.643 | −8.513 | 74.326 | 1.00 | 19.37 | Y | C |
| ATOM | 10863 | O | VAL | Y | 147 | −5.983 | −9.057 | 73.452 | 1.00 | 27.93 | Y | O |
| ATOM | 10864 | N | ASP | Y | 148 | −6.976 | −9.124 | 75.453 | 1.00 | 21.57 | Y | N |
| ATOM | 10865 | CA | ASP | Y | 148 | −6.595 | −10.510 | 75.728 | 1.00 | 25.70 | Y | C |
| ATOM | 10866 | CB | ASP | Y | 148 | −5.417 | −10.566 | 76.717 | 1.00 | 27.59 | Y | C |
| ATOM | 10867 | CG | ASP | Y | 148 | −4.170 | −9.868 | 76.179 | 1.00 | 38.46 | Y | C |
| ATOM | 10868 | OD1 | ASP | Y | 148 | −3.638 | −10.310 | 75.129 | 1.00 | 41.54 | Y | O |
| ATOM | 10869 | OD2 | ASP | Y | 148 | −3.743 | −8.857 | 76.782 | 1.00 | 39.91 | Y | O |
| ATOM | 10870 | C | ASP | Y | 148 | −7.786 | −11.255 | 76.317 | 1.00 | 28.56 | Y | C |
| ATOM | 10871 | O | ASP | Y | 148 | −8.634 | −10.663 | 76.981 | 1.00 | 26.45 | Y | O |
| ATOM | 10872 | N | TRP | Y | 149 | −7.847 | −12.557 | 76.084 | 1.00 | 28.24 | Y | N |
| ATOM | 10873 | CA | TRP | Y | 149 | −8.943 | −13.348 | 76.607 | 1.00 | 29.08 | Y | C |
| ATOM | 10874 | CB | TRP | Y | 149 | −9.760 | −13.943 | 75.468 | 1.00 | 28.65 | Y | C |
| ATOM | 10875 | CG | TRP | Y | 149 | −10.624 | −12.976 | 74.764 | 1.00 | 21.87 | Y | C |
| ATOM | 10876 | CD2 | TRP | Y | 149 | −12.028 | −12.827 | 74.931 | 1.00 | 23.92 | Y | C |
| ATOM | 10877 | CE2 | TRP | Y | 149 | −12.455 | −11.834 | 74.019 | 1.00 | 25.64 | Y | C |
| ATOM | 10878 | CE3 | TRP | Y | 149 | −12.971 | −13.432 | 75.764 | 1.00 | 19.03 | Y | C |
| ATOM | 10879 | CD1 | TRP | Y | 149 | −10.253 | −12.092 | 73.796 | 1.00 | 22.22 | Y | C |
| ATOM | 10880 | NE1 | TRP | Y | 149 | −11.351 | −11.402 | 73.332 | 1.00 | 25.62 | Y | N |
| ATOM | 10881 | CZ2 | TRP | Y | 149 | −13.780 | −11.437 | 73.920 | 1.00 | 23.85 | Y | C |
| ATOM | 10882 | CZ3 | TRP | Y | 149 | −14.287 | −13.033 | 75.667 | 1.00 | 23.03 | Y | C |
| ATOM | 10883 | CH2 | TRP | Y | 149 | −14.682 | −12.043 | 74.751 | 1.00 | 21.77 | Y | C |
| ATOM | 10884 | C | TRP | Y | 149 | −8.405 | −14.483 | 77.433 | 1.00 | 36.38 | Y | C |
| ATOM | 10885 | O | TRP | Y | 149 | −7.287 | −14.948 | 77.197 | 1.00 | 39.22 | Y | O |
| ATOM | 10886 | N | LYS | Y | 150 | −9.210 | −14.935 | 78.391 | 1.00 | 33.82 | Y | N |
| ATOM | 10887 | CA | LYS | Y | 150 | −8.842 | −16.053 | 79.241 | 1.00 | 37.29 | Y | C |
| ATOM | 10888 | CB | LYS | Y | 150 | −8.366 | −15.578 | 80.620 | 1.00 | 40.87 | Y | C |
| ATOM | 10889 | CG | LYS | Y | 150 | −7.107 | −14.723 | 80.651 | 1.00 | 41.85 | Y | C |
| ATOM | 10890 | CD | LYS | Y | 159 | −6.719 | −14.421 | 82.097 | 1.00 | 45.58 | Y | C |
| ATOM | 10891 | CE | LYS | Y | 150 | −7.908 | −13.854 | 82.890 | 1.00 | 49.69 | Y | C |
| ATOM | 10892 | NZ | LYS | Y | 150 | −7.582 | −13.406 | 84.292 | 1.00 | 54.65 | Y | N |
| ATOM | 10893 | C | LYS | Y | 150 | −10.048 | −16.968 | 79.448 | 1.00 | 40.83 | Y | C |
| ATOM | 10894 | O | LYS | Y | 150 | −11.192 | −16.514 | 79.462 | 1.00 | 40.37 | Y | O |
| ATOM | 10895 | N | ALA | Y | 151 | −9.779 | −18.263 | 79.583 | 1.00 | 41.69 | Y | N |
| ATOM | 10896 | CA | ALA | Y | 151 | −10.810 | −19.256 | 79.857 | 1.00 | 43.38 | Y | C |
| ATOM | 10897 | CB | ALA | Y | 151 | −11.043 | −20.179 | 78.644 | 1.00 | 38.72 | Y | C |
| ATOM | 10898 | C | ALA | Y | 151 | −10.308 | −20.052 | 81.073 | 1.00 | 45.03 | Y | C |
| ATOM | 10899 | O | ALA | Y | 151 | −9.234 | −20.664 | 81.032 | 1.00 | 40.24 | Y | O |
| ATOM | 10900 | N | ASP | Y | 152 | −11.044 | −19.957 | 82.181 | 1.00 | 52.10 | Y | N |
| ATOM | 10901 | CA | ASP | Y | 152 | −10.697 | −20.652 | 83.427 | 1.00 | 56.37 | Y | C |
| ATOM | 10902 | CB | ASP | Y | 152 | −10.805 | −22.182 | 83.252 | 1.00 | 57.19 | Y | C |
| ATOM | 10903 | CG | ASP | Y | 152 | −12.246 | −22.679 | 83.092 | 1.00 | 58.06 | Y | C |
| ATOM | 10904 | OD1 | ASP | Y | 152 | −13.211 | −21.995 | 83.513 | 1.00 | 53.59 | Y | O |
| ATOM | 10905 | OD2 | ASP | Y | 152 | −12.398 | −23.798 | 82.558 | 1.00 | 57.38 | Y | O |
| ATOM | 10906 | C | ASP | Y | 152 | −9.293 | −20.308 | 83.963 | 1.00 | 56.27 | Y | C |
| ATOM | 10907 | O | ASP | Y | 152 | −8.632 | −21.157 | 84.566 | 1.00 | 59.00 | Y | O |
| ATOM | 10908 | N | GLY | Y | 153 | −8.841 | −19.077 | 83.736 | 1.00 | 55.31 | Y | N |
| ATOM | 10909 | CA | GLY | Y | 153 | −7.529 | −18.668 | 84.208 | 1.00 | 55.75 | Y | C |
| ATOM | 10910 | C | GLY | Y | 153 | −6.370 | −18.904 | 83.252 | 1.00 | 52.41 | Y | C |
| ATOM | 10911 | O | GLY | Y | 153 | −5.238 | −18.555 | 83.567 | 1.00 | 59.44 | Y | O |
| ATOM | 10912 | N | THR | Y | 154 | −6.646 | −19.475 | 82.084 | 1.00 | 52.31 | Y | N |
| ATOM | 10913 | CA | THR | Y | 154 | −5.614 | −19.763 | 81.086 | 1.00 | 48.96 | Y | C |
| ATOM | 10914 | CB | THR | Y | 154 | −5.749 | −21.208 | 80.558 | 1.00 | 45.28 | Y | C |
| ATOM | 10915 | OG1 | THR | Y | 154 | −5.603 | −22.130 | 81.643 | 1.00 | 49.49 | Y | O |
| ATOM | 10916 | CG2 | THR | Y | 154 | −4.704 | −21.511 | 79.501 | 1.00 | 42.72 | Y | C |
| ATOM | 10917 | C | THR | Y | 154 | −5.800 | −18.854 | 79.894 | 1.00 | 50.29 | Y | C |
| ATOM | 10918 | O | THR | Y | 154 | −6.924 | −18.582 | 79.493 | 1.00 | 55.85 | Y | O |
| ATOM | 10919 | N | PRO | Y | 155 | −4.700 | −18.358 | 79.313 | 1.00 | 51.81 | Y | N |
| ATOM | 10920 | CD | PRO | Y | 155 | −3.345 | −18.338 | 79.891 | 1.00 | 50.60 | Y | C |
| ATOM | 10921 | CA | PRO | Y | 155 | −4.776 | −17.474 | 78.142 | 1.00 | 49.35 | Y | C |
| ATOM | 10922 | CB | PRO | Y | 155 | −3.319 | −17.041 | 77.952 | 1.00 | 46.20 | Y | C |
| ATOM | 10923 | CG | PRO | Y | 155 | −2.795 | −17.036 | 79.353 | 1.00 | 50.58 | Y | C |
| ATOM | 10924 | C | PRO | Y | 155 | −5.274 | −18.212 | 76.903 | 1.00 | 46.04 | Y | C |
| ATOM | 10925 | O | PRO | Y | 155 | −5.069 | −19.417 | 76.765 | 1.00 | 50.51 | Y | O |
| ATOM | 10926 | N | VAL | Y | 156 | −5.965 | −17.501 | 76.022 | 1.00 | 42.01 | Y | N |
| ATOM | 10927 | CA | VAL | Y | 156 | −6.431 | −18.117 | 74.796 | 1.00 | 42.28 | Y | C |
| ATOM | 10928 | CB | VAL | Y | 156 | −7.975 | −17.943 | 74.549 | 1.00 | 40.79 | Y | C |
| ATOM | 10929 | CG1 | VAL | Y | 156 | −8.643 | −17.238 | 75.712 | 1.00 | 36.47 | Y | C |
| ATOM | 10930 | CG2 | VAL | Y | 156 | −8.257 | −17.242 | 73.218 | 1.00 | 30.77 | Y | C |
| ATOM | 10931 | C | VAL | Y | 156 | −5.607 | −17.479 | 73.696 | 1.00 | 46.02 | Y | C |
| ATOM | 10932 | O | VAL | Y | 156 | −5.379 | −16.266 | 73.713 | 1.00 | 44.40 | Y | O |
| ATOM | 10933 | N | THR | Y | 157 | −5.124 | −18.312 | 72.775 | 1.00 | 47.68 | Y | N |
| ATOM | 10934 | CA | THR | Y | 157 | −4.304 | −17.850 | 71.658 | 1.00 | 48.47 | Y | C |
| ATOM | 10935 | CB | THR | Y | 157 | −2.950 | −18.590 | 71.605 | 1.00 | 51.43 | Y | C |
| ATOM | 10936 | OG1 | THR | Y | 157 | −3.175 | −19.998 | 71.438 | 1.00 | 57.44 | Y | O |
| ATOM | 10937 | CG2 | THR | Y | 157 | −2.165 | −18.352 | 72.892 | 1.00 | 50.60 | Y | C |
| ATOM | 10938 | C | THR | Y | 157 | −4.988 | −17.944 | 70.290 | 1.00 | 46.77 | Y | C |
| ATOM | 10939 | O | THR | Y | 157 | −4.486 | −17.402 | 69.313 | 1.00 | 52.70 | Y | O |
| ATOM | 10940 | N | GLN | Y | 158 | −6.113 | −18.641 | 70.208 | 1.00 | 45.69 | Y | N |
| ATOM | 10941 | CA | GLN | Y | 158 | −6.836 | −18.737 | 68.940 | 1.00 | 46.63 | Y | C |
| ATOM | 10942 | CB | GLN | Y | 158 | −6.551 | −20.066 | 68.221 | 1.00 | 48.23 | Y | C |
| ATOM | 10943 | CG | GLN | Y | 158 | −7.140 | −21.295 | 68.901 | 1.00 | 57.09 | Y | C |
| ATOM | 10944 | CD | GLN | Y | 158 | −7.305 | −22.474 | 67.956 | 1.00 | 63.38 | Y | C |
| ATOM | 10945 | OE1 | GLN | Y | 158 | −7.367 | −22.306 | 66.733 | 1.00 | 65.80 | Y | O |
| ATOM | 10946 | NE2 | GLN | Y | 158 | −7.405 | −23.679 | 68.523 | 1.00 | 66.81 | Y | N |
| ATOM | 10947 | C | GLN | Y | 158 | −8.342 | −18.544 | 69.151 | 1.00 | 45.19 | Y | C |
| ATOM | 10948 | O | GLN | Y | 158 | −8.856 | −18.727 | 70.259 | 1.00 | 46.42 | Y | O |
| ATOM | 10949 | N | GLY | Y | 159 | −9.037 | −18.136 | 68.092 | 1.00 | 43.60 | Y | N |
| ATOM | 10950 | CA | GLY | Y | 159 | −10.472 | −17.927 | 68.185 | 1.00 | 40.09 | Y | C |
| ATOM | 10951 | C | GLY | Y | 159 | −10.901 | −16.514 | 68.543 | 1.00 | 42.08 | Y | C |
| ATOM | 10952 | O | GLY | Y | 159 | −12.099 | −16.246 | 68.667 | 1.00 | 43.21 | Y | O |
| ATOM | 10953 | N | ASP | Y | 160 | −9.932 | −15.624 | 68.761 | 1.00 | 40.79 | Y | N |
| ATOM | 10954 | CA | ASP | Y | 160 | −10.226 | −14.233 | 69.085 | 1.00 | 37.75 | Y | C |
| ATOM | 10955 | CB | ASP | Y | 160 | −9.465 | −13.743 | 70.343 | 1.00 | 41.07 | Y | C |
| ATOM | 10956 | CG | ASP | Y | 160 | −7.972 | −14.170 | 70.398 | 1.00 | 49.40 | Y | C |
| ATOM | 10957 | OD1 | ASP | Y | 160 | −7.411 | −14.794 | 69.460 | 1.00 | 47.26 | Y | O |
| ATOM | 10958 | OD2 | ASP | Y | 160 | −7.352 | −13.870 | 71.444 | 1.00 | 47.49 | Y | O |
| ATOM | 10959 | C | ASP | Y | 160 | −10.022 | −13.296 | 67.899 | 1.00 | 40.40 | Y | C |
| ATOM | 10960 | O | ASP | Y | 160 | −9.213 | −13.569 | 67.015 | 1.00 | 45.29 | Y | O |
| ATOM | 10961 | N | GLU | Y | 161 | −10.801 | −12.218 | 67.856 | 1.00 | 43.92 | Y | N |
| ATOM | 10962 | CA | GLU | Y | 161 | −10.715 | −11.230 | 66.784 | 1.00 | 39.06 | Y | C |
| ATOM | 10963 | CB | GLU | Y | 161 | −11.676 | −11.606 | 65.653 | 1.00 | 44.44 | Y | C |
| ATOM | 10964 | CG | GLU | Y | 161 | −11.566 | −10.746 | 64.390 | 1.00 | 56.33 | Y | C |
| ATOM | 10965 | CD | GLU | Y | 161 | −10.357 | −11.099 | 63.528 | 1.00 | 63.10 | Y | C |
| ATOM | 10966 | OE1 | GLU | Y | 161 | −10.502 | −11.949 | 62.613 | 1.00 | 66.63 | Y | O |
| ATOM | 10967 | OE2 | GLU | Y | 161 | −9.265 | −10.529 | 63.767 | 1.00 | 66.92 | Y | O |
| ATOM | 10968 | C | GLU | Y | 161 | −11.102 | −9.869 | 67.363 | 1.00 | 38.14 | Y | C |
| ATOM | 10969 | O | GLU | Y | 161 | −12.178 | −9.735 | 67.948 | 1.00 | 40.82 | Y | O |
| ATOM | 10970 | N | THR | Y | 162 | −10.225 | −8.872 | 67.216 | 1.00 | 31.71 | Y | N |
| ATOM | 10971 | CA | THR | Y | 162 | −10.490 | −7.530 | 67.733 | 1.00 | 25.46 | Y | C |
| ATOM | 10972 | CB | THR | Y | 162 | −9.452 | −7.124 | 68.809 | 1.00 | 26.99 | Y | C |
| ATOM | 10973 | OG1 | THR | Y | 162 | −9.488 | −8.050 | 69.898 | 1.00 | 29.17 | Y | O |
| ATOM | 10974 | CG2 | THR | Y | 162 | −9.759 | −5.759 | 69.359 | 1.00 | 29.23 | Y | C |
| ATOM | 10975 | C | THR | Y | 162 | −10.482 | −6.489 | 66.626 | 1.00 | 29.14 | Y | C |
| ATOM | 10976 | O | THR | Y | 162 | −9.645 | −6.556 | 65.731 | 1.00 | 34.29 | Y | O |
| ATOM | 10977 | N | THR | Y | 163 | −11.438 | −5.554 | 66.659 | 1.00 | 24.84 | Y | N |
| ATOM | 10978 | CA | THR | Y | 163 | −11.503 | −4.491 | 65.659 | 1.00 | 25.41 | Y | C |
| ATOM | 10979 | CB | THR | Y | 163 | −12.899 | −3.848 | 65.580 | 1.00 | 29.17 | Y | C |
| ATOM | 10980 | OG1 | THR | Y | 163 | −13.320 | −3.420 | 66.887 | 1.00 | 25.73 | Y | O |
| ATOM | 10981 | CG2 | THR | Y | 163 | −13.912 | −4.837 | 64.994 | 1.00 | 31.58 | Y | C |
| ATOM | 10982 | C | THR | Y | 163 | −10.493 | −3.378 | 65.938 | 1.00 | 31.10 | Y | C |
| ATOM | 10983 | O | THR | Y | 163 | −10.035 | −3.193 | 67.068 | 1.00 | 36.63 | Y | O |
| ATOM | 10984 | N | GLN | Y | 164 | −10.132 | −2.646 | 64.897 | 1.00 | 32.20 | Y | N |
| ATOM | 10985 | CA | GLN | Y | 164 | −9.194 | −1.553 | 65.052 | 1.00 | 38.24 | Y | C |
| ATOM | 10986 | CB | GLN | Y | 164 | −8.606 | −1.180 | 63.683 | 1.00 | 46.05 | Y | C |
| ATOM | 10987 | CG | GLN | Y | 164 | −7.246 | −0.438 | 63.724 | 1.00 | 56.21 | Y | C |
| ATOM | 10988 | CD | GLN | Y | 164 | −6.079 | −1.296 | 64.247 | 1.00 | 59.79 | Y | C |
| ATOM | 10989 | OE1 | GLN | Y | 164 | −5.053 | −0.766 | 64.685 | 1.00 | 58.68 | Y | O |
| ATOM | 10990 | NE2 | GLN | Y | 164 | −6.230 | −2.621 | 64.187 | 1.00 | 59.18 | Y | N |
| ATOM | 10991 | C | GLN | Y | 164 | −9.983 | −0.389 | 65.666 | 1.00 | 36.21 | Y | C |
| ATOM | 10992 | O | GLN | Y | 164 | −11.133 | −0.152 | 65.307 | 1.00 | 38.37 | Y | O |
| ATOM | 10993 | N | PRO | Y | 165 | −9.402 | 0.305 | 66.652 | 1.00 | 32.77 | Y | N |
| ATOM | 10994 | CD | PRO | Y | 165 | −8.133 | −0.031 | 67.329 | 1.00 | 27.94 | Y | C |
| ATOM | 10995 | CA | PRO | Y | 165 | −10.077 | 1.436 | 67.304 | 1.00 | 28.50 | Y | C |
| ATOM | 10996 | CB | PRO | Y | 165 | −8.989 | 1.989 | 68.231 | 1.00 | 28.02 | Y | C |
| ATOM | 10997 | CG | PRO | Y | 165 | −8.216 | 0.758 | 68.606 | 1.00 | 22.05 | Y | C |
| ATOM | 10998 | C | PRO | Y | 165 | −10.602 | 2.507 | 66.340 | 1.00 | 33.02 | Y | C |
| ATOM | 10999 | O | PRO | Y | 165 | −9.923 | 2.892 | 65.387 | 1.00 | 36.36 | Y | O |
| ATOM | 11000 | N | SER | Y | 166 | −11.824 | 2.971 | 66.589 | 1.00 | 34.92 | Y | N |
| ATOM | 11001 | CA | SER | Y | 166 | −12.457 | 4.001 | 65.770 | 1.00 | 32.98 | Y | C |
| ATOM | 11002 | CB | SER | Y | 166 | −13.643 | 3.433 | 64.984 | 1.00 | 39.65 | Y | C |
| ATOM | 11003 | OG | SER | Y | 166 | −14.698 | 3.023 | 65.838 | 1.00 | 41.07 | Y | O |
| ATOM | 11004 | C | SER | Y | 166 | −12.940 | 5.080 | 66.717 | 1.00 | 33.87 | Y | C |
| ATOM | 11005 | O | SER | Y | 166 | −13.236 | 4.806 | 67.876 | 1.00 | 38.13 | Y | O |
| ATOM | 11006 | N | LYS | Y | 167 | −13.031 | 6.298 | 66.206 | 1.00 | 34.37 | Y | N |
| ATOM | 11007 | CA | LYS | Y | 167 | −13.438 | 7.475 | 66.964 | 1.00 | 34.57 | Y | C |
| ATOM | 11008 | CB | LYS | Y | 167 | −12.979 | 8.714 | 66.170 | 1.00 | 37.88 | Y | C |
| ATOM | 11009 | CG | LYS | Y | 167 | −12.383 | 9.873 | 66.959 | 1.00 | 39.59 | Y | C |
| ATOM | 11010 | CD | LYS | Y | 167 | −10.862 | 10.015 | 66.745 | 1.00 | 46.93 | Y | C |
| ATOM | 11011 | CE | LYS | Y | 167 | −10.445 | 10.488 | 65.324 | 1.00 | 50.99 | Y | C |
| ATOM | 11012 | NZ | LYS | Y | 167 | −10.561 | 11.966 | 65.071 | 1.00 | 46.26 | Y | N |
| ATOM | 11013 | C | LYS | Y | 167 | −14.950 | 7.574 | 67.207 | 1.00 | 34.44 | Y | C |
| ATOM | 11014 | O | LYS | Y | 167 | −15.742 | 7.410 | 66.282 | 1.00 | 36.31 | Y | O |
| ATOM | 11015 | N | GLN | Y | 168 | −15.339 | 7.858 | 68.451 | 1.00 | 35.28 | Y | N |
| ATOM | 11016 | CA | GLN | Y | 168 | −16.745 | 8.047 | 68.824 | 1.00 | 35.59 | Y | C |
| ATOM | 11017 | CB | GLN | Y | 168 | −16.954 | 7.788 | 70.323 | 1.00 | 34.42 | Y | C |
| ATOM | 11018 | CG | GLN | Y | 168 | −16.391 | 6.489 | 70.868 | 1.00 | 35.19 | Y | C |
| ATOM | 11019 | CD | GLN | Y | 168 | −16.703 | 6.278 | 72.353 | 1.00 | 33.57 | Y | C |
| ATOM | 11020 | OE1 | GLN | Y | 168 | −16.678 | 5.148 | 72.854 | 1.00 | 31.48 | Y | O |
| ATOM | 11021 | NE2 | GLN | Y | 168 | −16.980 | 7.364 | 73.061 | 1.00 | 31.15 | Y | N |
| ATOM | 11022 | C | GLN | Y | 168 | −17.077 | 9.522 | 68.559 | 1.00 | 35.05 | Y | C |
| ATOM | 11023 | O | GLN | Y | 168 | −16.208 | 10.288 | 68.144 | 1.00 | 40.31 | Y | O |
| ATOM | 11024 | N | SER | Y | 168A | −18.306 | 9.930 | 68.864 | 1.00 | 37.96 | Y | N |
| ATOM | 11025 | CA | SER | Y | 168A | −18.737 | 11.320 | 68.670 | 1.00 | 44.02 | Y | C |
| ATOM | 11026 | CB | SER | Y | 168A | −20.237 | 11.444 | 68.994 | 1.00 | 48.95 | Y | C |
| ATOM | 11027 | OG | SER | Y | 168A | −20.859 | 12.454 | 68.215 | 1.00 | 53.91 | Y | O |
| ATOM | 11028 | C | SER | Y | 168A | −17.931 | 12.237 | 69.604 | 1.00 | 43.59 | Y | C |
| ATOM | 11029 | O | SER | Y | 168A | −17.591 | 13.362 | 69.250 | 1.00 | 43.62 | Y | O |
| ATOM | 11030 | N | ASN | Y | 168B | −17.652 | 11.712 | 70.798 | 1.00 | 44.66 | Y | N |
| ATOM | 11031 | CA | ASN | Y | 168B | −16.894 | 12.339 | 71.892 | 1.00 | 42.07 | Y | C |
| ATOM | 11032 | CB | ASN | Y | 168B | −16.894 | 11.366 | 73.085 | 1.00 | 44.89 | Y | C |
| ATOM | 11033 | CG | ASN | Y | 168B | −17.806 | 11.795 | 74.207 | 1.00 | 51.41 | Y | C |
| ATOM | 11034 | OD1 | ASN | Y | 168B | −17.625 | 11.368 | 75.349 | 1.00 | 56.30 | Y | O |
| ATOM | 11035 | ND2 | ASN | Y | 168B | −18.808 | 12.617 | 73.895 | 1.00 | 55.09 | Y | N |
| ATOM | 11036 | C | ASN | Y | 168B | −15.423 | 12.550 | 71.555 | 1.00 | 39.31 | Y | C |
| ATOM | 11037 | O | ASN | Y | 168B | −14.674 | 13.140 | 72.336 | 1.00 | 35.79 | Y | O |
| ATOM | 11038 | N | ASN | Y | 169 | −15.004 | 11.934 | 70.461 | 1.00 | 34.71 | Y | N |
| ATOM | 11039 | CA | ASN | Y | 169 | −13.628 | 11.950 | 70.003 | 1.00 | 35.96 | Y | C |
| ATOM | 11040 | CB | ASN | Y | 169 | −13.038 | 13.362 | 69.970 | 1.00 | 43.63 | Y | C |
| ATOM | 11041 | CG | ASN | Y | 169 | −13.160 | 14.012 | 68.593 | 1.00 | 43.64 | Y | C |
| ATOM | 11042 | OD1 | ASN | Y | 169 | −12.914 | 13.375 | 67.568 | 1.00 | 42.26 | Y | O |
| ATOM | 11043 | ND2 | ASN | Y | 169 | −13.533 | 15.286 | 68.571 | 1.00 | 42.02 | Y | N |
| ATOM | 11044 | C | ASN | Y | 169 | −12.760 | 10.954 | 70.784 | 1.00 | 36.16 | Y | C |
| ATOM | 11045 | O | ASN | Y | 169 | −11.549 | 10.853 | 70.554 | 1.00 | 43.19 | Y | O |
| ATOM | 11046 | N | LYS | Y | 170 | −13.368 | 10.236 | 71.728 | 1.00 | 30.27 | Y | N |
| ATOM | 11047 | CA | LYS | Y | 170 | −12.637 | 9.187 | 72.436 | 1.00 | 26.97 | Y | C |
| ATOM | 11048 | CB | LYS | Y | 170 | −13.267 | 8.844 | 73.784 | 1.00 | 25.58 | Y | C |
| ATOM | 11049 | CG | LYS | Y | 170 | −13.358 | 10.007 | 74.741 | 1.00 | 25.97 | Y | C |
| ATOM | 11050 | CD | LYS | Y | 170 | −13.674 | 9.544 | 76.154 | 1.00 | 29.59 | Y | C |
| ATOM | 11051 | CE | LYS | Y | 170 | −14.067 | 10.734 | 77.028 | 1.00 | 31.93 | Y | C |
| ATOM | 11052 | NZ | LYS | Y | 170 | −13.965 | 10.383 | 78.479 | 1.00 | 36.72 | Y | N |
| ATOM | 11053 | C | LYS | Y | 170 | −12.777 | 7.999 | 71.481 | 1.00 | 29.15 | Y | C |
| ATOM | 11054 | O | LYS | Y | 170 | −13.342 | 8.143 | 70.402 | 1.00 | 27.04 | Y | O |
| ATOM | 11055 | N | TYR | Y | 171 | −12.286 | 6.824 | 71.851 | 1.00 | 29.28 | Y | N |
| ATOM | 11056 | CA | TYR | Y | 171 | −12.392 | 5.698 | 70.939 | 1.00 | 24.06 | Y | C |
| ATOM | 11057 | CB | TYR | Y | 171 | −11.015 | 5.226 | 70.523 | 1.00 | 28.25 | Y | C |
| ATOM | 11058 | CG | TYR | Y | 171 | −10.239 | 6.198 | 69.678 | 1.00 | 30.73 | Y | C |
| ATOM | 11059 | CD1 | TYR | Y | 171 | −9.725 | 7.379 | 70.224 | 1.00 | 31.01 | Y | C |
| ATOM | 11060 | CE1 | TYR | Y | 171 | −8.932 | 8.231 | 69.473 | 1.00 | 29.13 | Y | C |
| ATOM | 11061 | CD2 | TYR | Y | 171 | −9.940 | 5.901 | 68.347 | 1.00 | 32.35 | Y | C |
| ATOM | 11062 | CE2 | TYR | Y | 171 | −9.137 | 6.753 | 67.583 | 1.00 | 34.65 | Y | C |
| ATOM | 11063 | CZ | TYR | Y | 171 | −8.636 | 7.913 | 68.155 | 1.00 | 31.12 | Y | C |
| ATOM | 11064 | OH | TYR | Y | 171 | −7.824 | 8.739 | 67.415 | 1.00 | 36.21 | Y | O |
| ATOM | 11065 | C | TYR | Y | 171 | −13.187 | 4.511 | 71.452 | 1.00 | 29.42 | Y | C |
| ATOM | 11066 | O | TYR | Y | 171 | −13.420 | 4.371 | 72.673 | 1.00 | 19.73 | Y | O |
| ATOM | 11067 | N | MET | Y | 172 | −13.611 | 3.668 | 70.503 | 1.00 | 20.46 | Y | N |
| ATOM | 11068 | CA | MET | Y | 172 | −14.368 | 2.463 | 70.811 | 1.00 | 19.67 | Y | C |
| ATOM | 11069 | CB | MET | Y | 172 | −15.819 | 2.598 | 70.379 | 1.00 | 26.52 | Y | C |
| ATOM | 11070 | CG | MET | Y | 172 | −16.022 | 2.577 | 68.886 | 1.00 | 26.35 | Y | C |
| ATOM | 11071 | SD | MET | Y | 172 | −17.738 | 2.804 | 68.444 | 1.00 | 38.09 | Y | S |
| ATOM | 11072 | CE | MET | Y | 172 | −17.903 | 4.576 | 68.669 | 1.00 | 23.54 | Y | C |
| ATOM | 11073 | C | MET | Y | 172 | −13.731 | 1.350 | 70.039 | 1.00 | 18.16 | Y | C |
| ATOM | 11074 | O | MET | Y | 172 | −12.985 | 1.594 | 69.106 | 1.00 | 28.87 | Y | O |
| ATOM | 11075 | N | ALA | Y | 173 | −14.037 | 0.121 | 70.414 | 1.00 | 18.21 | Y | N |
| ATOM | 11076 | CA | ALA | Y | 173 | −13.477 | −1.039 | 69.747 | 1.00 | 20.46 | Y | C |
| ATOM | 11077 | CB | ALA | Y | 173 | −11.987 | −1.171 | 70.087 | 1.00 | 18.64 | Y | C |
| ATOM | 11078 | C | ALA | Y | 173 | −14.245 | −2.261 | 70.215 | 1.00 | 18.61 | Y | C |
| ATOM | 11079 | O | ALA | Y | 173 | −15.022 | −2.159 | 71.145 | 1.00 | 17.32 | Y | O |
| ATOM | 11080 | N | SER | Y | 174 | −14.010 | −3.417 | 69.603 | 1.00 | 15.99 | Y | N |
| ATOM | 11081 | CA | SER | Y | 174 | −14.731 | −4.621 | 69.993 | 1.00 | 21.40 | Y | C |
| ATOM | 11082 | CB | SER | Y | 174 | −16.054 | −4.740 | 69.221 | 1.00 | 21.29 | Y | C |
| ATOM | 11083 | OG | SER | Y | 174 | −15.819 | −4.853 | 67.818 | 1.00 | 26.30 | Y | O |
| ATOM | 11084 | C | SER | Y | 174 | −13.896 | −5.864 | 69.749 | 1.00 | 25.23 | Y | C |
| ATOM | 11085 | O | SER | Y | 174 | −13.076 | −5.902 | 68.826 | 1.00 | 26.80 | Y | O |
| ATOM | 11086 | N | SER | Y | 175 | −14.134 | −6.888 | 70.568 | 1.00 | 19.21 | Y | N |
| ATOM | 11087 | CA | SER | Y | 175 | −13.413 | −8.144 | 70.453 | 1.00 | 20.94 | Y | C |
| ATOM | 11088 | CB | SER | Y | 175 | −12.343 | −8.247 | 71.550 | 1.00 | 19.27 | Y | C |
| ATOM | 11089 | OG | SER | Y | 175 | −11.605 | −9.455 | 71.435 | 1.00 | 16.96 | Y | O |
| ATOM | 11090 | C | SER | Y | 175 | −14.397 | −9.311 | 70.563 | 1.00 | 22.64 | Y | C |
| ATOM | 11091 | O | SER | Y | 175 | −15.364 | −9.268 | 71.328 | 1.00 | 23.08 | Y | O |
| ATOM | 11092 | N | TYR | Y | 176 | −14.103 | −10.378 | 69.837 | 1.00 | 23.46 | Y | N |
| ATOM | 11093 | CA | TYR | Y | 176 | −14.957 | −11.552 | 69.821 | 1.00 | 24.38 | Y | C |
| ATOM | 11094 | CB | TYR | Y | 176 | −15.552 | −11.728 | 68.414 | 1.00 | 21.51 | Y | C |
| ATOM | 11095 | CG | TYR | Y | 176 | −16.405 | −10.584 | 67.967 | 1.00 | 20.85 | Y | C |
| ATOM | 11096 | CD1 | TYR | Y | 176 | −17.725 | −10.479 | 68.395 | 1.00 | 28.82 | Y | C |
| ATOM | 11097 | CE1 | TYR | Y | 176 | −18.542 | −9.451 | 67.968 | 1.00 | 28.25 | Y | C |
| ATOM | 11098 | CD2 | TYR | Y | 176 | −15.916 | −9.627 | 67.102 | 1.00 | 22.51 | Y | C |
| ATOM | 11099 | CE2 | TYR | Y | 176 | −16.724 | −8.585 | 66.664 | 1.00 | 25.87 | Y | C |
| ATOM | 11100 | CZ | TYR | Y | 176 | −18.039 | −8.508 | 67.102 | 1.00 | 29.77 | Y | C |
| ATOM | 11101 | OH | TYR | Y | 176 | −18.865 | −7.491 | 66.682 | 1.00 | 36.77 | Y | O |
| ATOM | 11102 | C | TYR | Y | 176 | −14.232 | −12.835 | 70.166 | 1.00 | 26.33 | Y | C |
| ATOM | 11103 | O | TYR | Y | 176 | −13.120 | −13.072 | 69.689 | 1.00 | 25.61 | Y | O |
| ATOM | 11104 | N | LEU | Y | 177 | −14.876 | −13.678 | 70.971 | 1.00 | 24.83 | Y | N |
| ATOM | 11105 | CA | LEU | Y | 177 | −14.302 | −14.977 | 71.281 | 1.00 | 26.73 | Y | C |
| ATOM | 11106 | CB | LEU | Y | 177 | −14.162 | −15.199 | 72.784 | 1.00 | 26.84 | Y | C |
| ATOM | 11107 | CG | LEU | Y | 177 | −13.628 | −16.596 | 73.104 | 1.00 | 23.85 | Y | C |
| ATOM | 11108 | CD1 | LEU | Y | 177 | −12.411 | −16.888 | 72.233 | 1.00 | 20.80 | Y | C |
| ATOM | 11109 | CD2 | LEU | Y | 177 | −13.294 | −16.703 | 74.574 | 1.00 | 23.81 | Y | C |
| ATOM | 11110 | C | LEU | Y | 177 | −15.248 | −16.014 | 70.669 | 1.00 | 29.41 | Y | C |
| ATOM | 11111 | O | LEU | Y | 177 | −16.396 | −16.140 | 71.081 | 1.00 | 28.20 | Y | O |
| ATOM | 11112 | N | THR | Y | 178 | −14.766 | −16.724 | 69.657 | 1.00 | 31.74 | Y | N |
| ATOM | 11113 | CA | THR | Y | 178 | −15.572 | −17.717 | 68.963 | 1.00 | 32.55 | Y | C |
| ATOM | 11114 | CB | THR | Y | 178 | −15.404 | −17.572 | 67.442 | 1.00 | 34.56 | Y | C |
| ATOM | 11115 | OG1 | THR | Y | 178 | −16.020 | −16.345 | 67.018 | 1.00 | 32.79 | Y | O |
| ATOM | 11116 | CG2 | THR | Y | 178 | −16.028 | −18.751 | 66.711 | 1.00 | 31.59 | Y | C |
| ATOM | 11117 | C | THR | Y | 178 | −15.276 | −19.139 | 69.387 | 1.00 | 37.54 | Y | C |
| ATOM | 11118 | O | THR | Y | 178 | −14.178 | −19.653 | 69.172 | 1.00 | 43.75 | Y | O |
| ATOM | 11119 | N | LEU | Y | 179 | −16.277 | −19.775 | 69.983 | 1.00 | 40.71 | Y | N |
| ATOM | 11120 | CA | LEU | Y | 179 | −16.158 | −21.147 | 70.459 | 1.00 | 39.02 | Y | C |
| ATOM | 11121 | CB | LEU | Y | 179 | −16.229 | −21.184 | 71.981 | 1.00 | 44.73 | Y | C |
| ATOM | 11122 | CG | LEU | Y | 179 | −15.378 | −20.247 | 72.828 | 1.00 | 46.69 | Y | C |
| ATOM | 11123 | CD1 | LEU | Y | 179 | −16.000 | −20.188 | 74.203 | 1.00 | 47.13 | Y | C |
| ATOM | 11124 | CD2 | LEU | Y | 179 | −13.934 | −20.720 | 72.898 | 1.00 | 44.49 | Y | C |
| ATOM | 11125 | C | LEU | Y | 179 | −17.345 | −21.932 | 69.959 | 1.00 | 41.70 | Y | C |
| ATOM | 11126 | O | LEU | Y | 179 | −18.321 | −21.359 | 69.468 | 1.00 | 46.05 | Y | O |
| ATOM | 11127 | N | THR | Y | 180 | −17.263 | −23.249 | 70.092 | 1.00 | 39.48 | Y | N |
| ATOM | 11128 | CA | THR | Y | 180 | −18.371 | −24.113 | 69.714 | 1.00 | 37.86 | Y | C |
| ATOM | 11129 | CB | THR | Y | 180 | −17.916 | −25.545 | 69.427 | 1.00 | 41.26 | Y | C |
| ATOM | 11130 | OG1 | THR | Y | 180 | −17.141 | −26.034 | 70.538 | 1.00 | 39.44 | Y | O |
| ATOM | 11131 | CG2 | THR | Y | 180 | −17.109 | −25.605 | 68.129 | 1.00 | 39.78 | Y | C |
| ATOM | 11132 | C | THR | Y | 180 | −19.226 | −24.168 | 70.961 | 1.00 | 38.08 | Y | C |
| ATOM | 11133 | O | THR | Y | 180 | −18.767 | −23.799 | 72.040 | 1.00 | 42.31 | Y | O |
| ATOM | 11134 | N | ALA | Y | 181 | −20.459 | −24.642 | 70.830 | 1.00 | 43.36 | Y | N |
| ATOM | 11135 | CA | ALA | Y | 181 | −21.336 | −24.760 | 71.991 | 1.00 | 41.35 | Y | C |
| ATOM | 11136 | CB | ALA | Y | 181 | −22.722 | −25.242 | 71.581 | 1.00 | 43.31 | Y | C |
| ATOM | 11137 | C | ALA | Y | 181 | −20.709 | −25.741 | 72.971 | 1.00 | 42.22 | Y | C |
| ATOM | 11138 | O | ALA | Y | 181 | −20.877 | −25.596 | 74.174 | 1.00 | 44.30 | Y | O |
| ATOM | 11139 | N | GLU | Y | 182 | −19.992 | −26.736 | 72.447 | 1.00 | 42.13 | Y | N |
| ATOM | 11140 | CA | GLU | Y | 182 | −19.311 | −27.735 | 73.275 | 1.00 | 52.06 | Y | C |
| ATOM | 11141 | CB | GLU | Y | 182 | −18.471 | −28.691 | 72.413 | 1.00 | 59.46 | Y | C |
| ATOM | 11142 | CG | GLU | Y | 182 | −19.189 | −29.615 | 71.440 | 1.00 | 67.00 | Y | C |
| ATOM | 11143 | CD | GLU | Y | 182 | −18.200 | −30.527 | 70.703 | 1.00 | 70.57 | Y | C |
| ATOM | 11144 | OE1 | GLU | Y | 182 | −17.174 | −30.005 | 70.192 | 1.00 | 71.81 | Y | O |
| ATOM | 11145 | OE2 | GLU | Y | 182 | −18.437 | −31.760 | 70.649 | 1.00 | 70.69 | Y | O |
| ATOM | 11146 | C | GLU | Y | 182 | −18.326 | −27.049 | 74.220 | 1.00 | 49.44 | Y | C |
| ATOM | 11147 | O | GLU | Y | 182 | −18.365 | −27.237 | 75.442 | 1.00 | 50.60 | Y | O |
| ATOM | 11148 | N | ALA | Y | 183 | −17.406 | −26.298 | 73.613 | 1.00 | 48.89 | Y | N |
| ATOM | 11149 | CA | ALA | Y | 183 | −16.364 | −25.568 | 74.326 | 1.00 | 46.39 | Y | C |
| ATOM | 11150 | CB | ALA | Y | 183 | −15.542 | −24.746 | 73.337 | 1.00 | 45.29 | Y | C |
| ATOM | 11151 | C | ALA | Y | 183 | −16.959 | −24.668 | 75.393 | 1.00 | 41.18 | Y | C |
| ATOM | 11152 | O | ALA | Y | 183 | −16.483 | −24.643 | 76.521 | 1.00 | 40.66 | Y | O |
| ATOM | 11153 | N | TRP | Y | 184 | −18.007 | −23.940 | 75.025 | 1.00 | 33.55 | Y | N |
| ATOM | 11154 | CA | TRP | Y | 184 | −18.668 | −23.052 | 75.958 | 1.00 | 37.64 | Y | C |
| ATOM | 11155 | CB | TRP | Y | 184 | −19.818 | −22.339 | 75.260 | 1.00 | 31.64 | Y | C |
| ATOM | 11156 | CG | TRP | Y | 184 | −20.806 | −21.715 | 76.195 | 1.00 | 32.04 | Y | C |
| ATOM | 11157 | CD2 | TRP | Y | 184 | −20.654 | −20.496 | 76.941 | 1.00 | 31.34 | Y | C |
| ATOM | 11158 | CE2 | TRP | Y | 184 | −21.852 | −20.297 | 77.662 | 1.00 | 29.34 | Y | C |
| ATOM | 11159 | CE3 | TRP | Y | 184 | −19.622 | −19.555 | 77.071 | 1.00 | 33.51 | Y | C |
| ATOM | 11160 | CD1 | TRP | Y | 184 | −22.045 | −22.189 | 76.489 | 1.00 | 35.00 | Y | C |
| ATOM | 11161 | NE1 | TRP | Y | 184 | −22.682 | −21.344 | 77.368 | 1.00 | 38.43 | Y | N |
| ATOM | 11162 | CZ2 | TRP | Y | 184 | −22.056 | −19.203 | 78.495 | 1.00 | 23.94 | Y | C |
| ATOM | 11163 | CZ3 | TRP | Y | 184 | −19.823 | −18.461 | 77.905 | 1.00 | 29.11 | Y | C |
| ATOM | 11164 | CH2 | TRP | Y | 184 | −21.037 | −18.296 | 78.607 | 1.00 | 34.75 | Y | C |
| ATOM | 11165 | C | TRP | Y | 184 | −19.175 | −23.786 | 77.194 | 1.00 | 41.03 | Y | C |
| ATOM | 11166 | O | TRP | Y | 184 | −19.073 | −23.276 | 78.308 | 1.00 | 44.78 | Y | O |
| ATOM | 11167 | N | GLU | Y | 185 | −19.721 | −24.979 | 76.986 | 1.00 | 46.79 | Y | N |
| ATOM | 11168 | CA | GLU | Y | 185 | −20.263 | −25.788 | 78.074 | 1.00 | 52.74 | Y | C |
| ATOM | 11169 | CB | GLU | Y | 185 | −21.272 | −26.807 | 77.515 | 1.00 | 56.21 | Y | C |
| ATOM | 11170 | CG | GLU | Y | 185 | −22.556 | −26.168 | 76.943 | 1.00 | 58.28 | Y | C |
| ATOM | 11171 | CD | GLU | Y | 185 | −23.383 | −27.118 | 76.072 | 1.00 | 61.25 | Y | C |
| ATOM | 11172 | OE1 | GLU | Y | 185 | −23.276 | −28.348 | 76.264 | 1.00 | 63.17 | Y | O |
| ATOM | 11173 | OE2 | GLU | Y | 185 | −24.141 | −26.634 | 75.192 | 1.00 | 57.26 | Y | O |
| ATOM | 11174 | C | GLU | Y | 185 | −19.186 | −26.478 | 78.924 | 1.00 | 53.47 | Y | C |
| ATOM | 11175 | O | GLU | Y | 185 | −19.385 | −26.695 | 80.119 | 1.00 | 52.59 | Y | O |
| ATOM | 11176 | N | SER | Y | 186 | −18.044 | −26.786 | 78.309 | 1.00 | 52.83 | Y | N |
| ATOM | 11177 | CA | SER | Y | 186 | −16.917 | −27.448 | 78.985 | 1.00 | 55.99 | Y | C |
| ATOM | 11178 | CB | SER | Y | 186 | −15.856 | −27.911 | 77.958 | 1.00 | 56.74 | Y | C |
| ATOM | 11179 | OG | SER | Y | 186 | −16.112 | −29.208 | 77.425 | 1.00 | 51.80 | Y | O |
| ATOM | 11180 | C | SER | Y | 186 | −16.216 | −26.570 | 80.026 | 1.00 | 56.08 | Y | C |
| ATOM | 11181 | O | SER | Y | 186 | −15.774 | −27.055 | 81.069 | 1.00 | 62.46 | Y | O |
| ATOM | 11182 | N | HIS | Y | 187 | −16.088 | −25.285 | 79.725 | 1.00 | 54.51 | Y | N |
| ATOM | 11183 | CA | HIS | Y | 187 | −15.413 | −24.349 | 80.616 | 1.00 | 52.04 | Y | C |
| ATOM | 11184 | CB | HIS | Y | 187 | −14.479 | −23.469 | 79.786 | 1.00 | 50.56 | Y | C |
| ATOM | 11185 | CG | HIS | Y | 187 | −13.504 | −24.246 | 78.950 | 1.00 | 52.64 | Y | C |
| ATOM | 11186 | CD2 | HIS | Y | 187 | −12.271 | −24.733 | 79.235 | 1.00 | 53.91 | Y | C |
| ATOM | 11187 | ND1 | HIS | Y | 187 | −13.772 | −24.625 | 77.651 | 1.00 | 56.62 | Y | N |
| ATOM | 11188 | CE1 | HIS | Y | 187 | −12.749 | −25.313 | 77.172 | 1.00 | 55.04 | Y | C |
| ATOM | 11189 | NE2 | HIS | Y | 187 | −11.824 | −25.393 | 78.113 | 1.00 | 54.70 | Y | N |
| ATOM | 11190 | C | HIS | Y | 187 | −16.416 | −23.517 | 81.417 | 1.00 | 53.75 | Y | C |
| ATOM | 11191 | O | HIS | Y | 187 | −17.592 | −23.452 | 81.055 | 1.00 | 62.33 | Y | O |
| ATOM | 11192 | N | SER | Y | 188 | −15.970 | −22.885 | 82.502 | 1.00 | 55.30 | Y | N |
| ATOM | 11193 | CA | SER | Y | 188 | −16.879 | −22.089 | 83.332 | 1.00 | 53.03 | Y | C |
| ATOM | 11194 | CB | SER | Y | 188 | −17.039 | −22.745 | 84.709 | 1.00 | 54.83 | Y | C |
| ATOM | 11195 | OG | SER | Y | 188 | −15.777 | −22.979 | 85.314 | 1.00 | 56.50 | Y | O |
| ATOM | 11196 | C | SER | Y | 188 | −16.556 | −20.608 | 83.511 | 1.00 | 50.29 | Y | C |
| ATOM | 11197 | O | SER | Y | 188 | −17.426 | −19.822 | 83.889 | 1.00 | 59.73 | Y | O |
| ATOM | 11198 | N | SER | Y | 189 | −15.328 | −20.216 | 83.213 | 1.00 | 47.05 | Y | N |
| ATOM | 11199 | CA | SER | Y | 189 | −14.929 | −18.829 | 83.395 | 1.00 | 45.14 | Y | C |
| ATOM | 11200 | CB | SER | Y | 189 | −13.875 | −18.753 | 84.504 | 1.00 | 45.59 | Y | C |
| ATOM | 11201 | OG | SER | Y | 189 | −13.339 | −17.446 | 84.618 | 1.00 | 50.47 | Y | O |
| ATOM | 11202 | C | SER | Y | 189 | −14.373 | −18.194 | 82.130 | 1.00 | 41.86 | Y | C |
| ATOM | 11203 | O | SER | Y | 189 | −13.492 | −18.757 | 81.488 | 1.00 | 37.91 | Y | O |
| ATOM | 11204 | N | TYR | Y | 190 | −14.873 | −17.009 | 81.789 | 1.00 | 36.30 | Y | N |
| ATOM | 11205 | CA | TYR | Y | 190 | −14.398 | −16.286 | 80.615 | 1.00 | 36.83 | Y | C |
| ATOM | 11206 | CB | TYR | Y | 190 | −15.439 | −16.327 | 79.486 | 1.00 | 42.31 | Y | C |
| ATOM | 11207 | CG | TYR | Y | 190 | −15.628 | −17.717 | 78.938 | 1.00 | 41.79 | Y | C |
| ATOM | 11208 | CD1 | TYR | Y | 190 | −16.551 | −18.589 | 79.513 | 1.00 | 43.35 | Y | C |
| ATOM | 11209 | CE1 | TYR | Y | 190 | −16.669 | −19.895 | 79.078 | 1.00 | 43.15 | Y | C |
| ATOM | 11210 | CD2 | TYR | Y | 190 | −14.832 | −18.186 | 77.907 | 1.00 | 38.59 | Y | C |
| ATOM | 11211 | CE2 | TYR | Y | 190 | −14.939 | −19.488 | 77.463 | 1.00 | 42.21 | Y | C |
| ATOM | 11212 | CZ | TYR | Y | 190 | −15.860 | −20.341 | 78.052 | 1.00 | 46.46 | Y | C |
| ATOM | 11213 | OH | TYR | Y | 190 | −15.970 | −21.649 | 77.619 | 1.00 | 52.78 | Y | O |
| ATOM | 11214 | C | TYR | Y | 190 | −14.066 | −14.856 | 80.986 | 1.00 | 28.97 | Y | C |
| ATOM | 11215 | O | TYR | Y | 190 | −14.831 | −14.193 | 81.682 | 1.00 | 32.47 | Y | O |
| ATOM | 11216 | N | SER | Y | 191 | −12.926 | −14.383 | 80.499 | 1.00 | 31.03 | Y | N |
| ATOM | 11217 | CA | SER | Y | 191 | −12.463 | −13.032 | 80.791 | 1.00 | 28.48 | Y | C |
| ATOM | 11218 | CB | SER | Y | 191 | −11.313 | −13.077 | 81.801 | 1.00 | 27.83 | Y | C |
| ATOM | 11219 | OG | SER | Y | 191 | −11.785 | −13.363 | 83.100 | 1.00 | 35.15 | Y | O |
| ATOM | 11220 | C | SER | Y | 191 | −11.990 | −12.268 | 79.585 | 1.00 | 22.02 | Y | C |
| ATOM | 11221 | O | SER | Y | 191 | −11.266 | −12.790 | 78.742 | 1.00 | 30.35 | Y | O |
| ATOM | 11222 | N | CYS | Y | 192 | −12.384 | −11.009 | 79.527 | 1.00 | 21.36 | Y | N |
| ATOM | 11223 | CA | CYS | Y | 192 | −11.970 | −10.130 | 78.458 | 1.00 | 21.88 | Y | C |
| ATOM | 11224 | C | CYS | Y | 192 | −11.105 | −9.074 | 79.152 | 1.00 | 26.71 | Y | C |
| ATOM | 11225 | O | CYS | Y | 192 | −11.591 | −8.346 | 80.040 | 1.00 | 25.87 | Y | O |
| ATOM | 11226 | CB | CYS | Y | 192 | −13.172 | −9.455 | 77.792 | 1.00 | 15.02 | Y | C |
| ATOM | 11227 | SG | CYS | Y | 192 | −12.629 | −8.309 | 76.492 | 1.00 | 31.43 | Y | S |
| ATOM | 11228 | N | HIS | Y | 193 | −9.822 | −9.050 | 78.783 | 1.00 | 30.79 | Y | N |
| ATOM | 11229 | CA | HIS | Y | 193 | −8.816 | −8.112 | 79.306 | 1.00 | 27.46 | Y | C |
| ATOM | 11230 | CB | HIS | Y | 193 | −7.444 | −8.769 | 79.399 | 1.00 | 35.32 | Y | C |
| ATOM | 11231 | CG | HIS | Y | 193 | −7.235 | −9.680 | 80.563 | 1.00 | 37.16 | Y | C |
| ATOM | 11232 | CD2 | HIS | Y | 193 | −7.638 | −9.601 | 81.851 | 1.00 | 40.09 | Y | C |
| ATOM | 11233 | ND1 | HIS | Y | 193 | −6.342 | −10.733 | 80.505 | 1.00 | 37.00 | Y | N |
| ATOM | 11234 | CE1 | HIS | Y | 193 | −6.192 | −11.248 | 81.711 | 1.00 | 37.23 | Y | C |
| ATOM | 11235 | NE2 | HIS | Y | 193 | −6.967 | −10.581 | 82.547 | 1.00 | 38.05 | Y | N |
| ATOM | 11236 | C | HIS | Y | 193 | −8.594 | −7.008 | 78.274 | 1.00 | 27.73 | Y | C |
| ATOM | 11237 | O | HIS | Y | 193 | −8.305 | −7.285 | 77.103 | 1.00 | 27.30 | Y | O |
| ATOM | 11238 | N | VAL | Y | 194 | −8.621 | −5.765 | 78.726 | 1.00 | 22.36 | Y | N |
| ATOM | 11239 | CA | VAL | Y | 194 | −8.369 | −4.635 | 77.849 | 1.00 | 22.69 | Y | C |
| ATOM | 11240 | CB | VAL | Y | 194 | −9.655 | −3.829 | 77.590 | 1.00 | 14.74 | Y | C |
| ATOM | 11241 | CG1 | VAL | Y | 194 | −9.383 | −2.677 | 76.658 | 1.00 | 12.59 | Y | C |
| ATOM | 11242 | CG2 | VAL | Y | 194 | −10.715 | −4.735 | 76.982 | 1.00 | 20.98 | Y | C |
| ATOM | 11243 | C | VAL | Y | 194 | −7.319 | −3.764 | 78.536 | 1.00 | 26.62 | Y | C |
| ATOM | 11244 | O | VAL | Y | 194 | −7.578 | −3.199 | 79.596 | 1.00 | 30.43 | Y | O |
| ATOM | 11245 | N | THR | Y | 195 | −6.115 | −3.722 | 77.970 | 1.00 | 26.28 | Y | N |
| ATOM | 11246 | CA | THR | Y | 195 | −5.025 | −2.925 | 78.529 | 1.00 | 27.39 | Y | C |
| ATOM | 11247 | CB | THR | Y | 195 | −3.663 | −3.693 | 78.523 | 1.00 | 30.06 | Y | C |
| ATOM | 11248 | OG1 | THR | Y | 195 | −3.741 | −4.833 | 79.394 | 1.00 | 29.09 | Y | O |
| ATOM | 11249 | CG2 | THR | Y | 195 | −2.520 | −2.779 | 78.997 | 1.00 | 20.59 | Y | C |
| ATOM | 11250 | C | THR | Y | 195 | −4.860 | −1.591 | 77.803 | 1.00 | 29.65 | Y | C |
| ATOM | 11251 | O | THR | Y | 195 | −4.634 | −1.548 | 76.594 | 1.00 | 23.36 | Y | O |
| ATOM | 11252 | N | HIS | Y | 196 | −4.955 | −0.514 | 78.583 | 1.00 | 27.90 | Y | N |
| ATOM | 11253 | CA | HIS | Y | 196 | −4.835 | 0.857 | 78.107 | 1.00 | 29.39 | Y | C |
| ATOM | 11254 | CB | HIS | Y | 196 | −6.158 | 1.595 | 78.304 | 1.00 | 26.66 | Y | C |
| ATOM | 11255 | CG | HIS | Y | 196 | −6.174 | 2.977 | 77.726 | 1.00 | 22.66 | Y | C |
| ATOM | 11256 | CD2 | HIS | Y | 196 | −6.024 | 3.413 | 76.453 | 1.00 | 21.18 | Y | C |
| ATOM | 11257 | ND1 | HIS | Y | 196 | −6.380 | 4.099 | 78.492 | 1.00 | 21.53 | Y | N |
| ATOM | 11258 | CE1 | HIS | Y | 196 | −6.359 | 5.168 | 77.718 | 1.00 | 21.94 | Y | C |
| ATOM | 11259 | NE2 | HIS | Y | 196 | −6.144 | 4.779 | 76.476 | 1.00 | 22.25 | Y | N |
| ATOM | 11260 | C | HIS | Y | 196 | −3.767 | 1.547 | 78.944 | 1.00 | 33.54 | Y | C |
| ATOM | 11261 | O | HIS | Y | 196 | −3.877 | 1.600 | 80.173 | 1.00 | 34.00 | Y | O |
| ATOM | 11262 | N | GLU | Y | 197 | −2.753 | 2.081 | 78.266 | 1.00 | 34.35 | Y | N |
| ATOM | 11263 | CA | GLU | Y | 197 | −1.632 | 2.752 | 78.910 | 1.00 | 37.42 | Y | C |
| ATOM | 11264 | CB | GLU | Y | 197 | −2.019 | 4.160 | 79.323 | 1.00 | 42.51 | Y | C |
| ATOM | 11265 | CG | GLU | Y | 197 | −2.087 | 5.078 | 78.141 | 1.00 | 55.18 | Y | C |
| ATOM | 11266 | CD | GLU | Y | 197 | −2.199 | 6.514 | 78.551 | 1.00 | 63.90 | Y | C |
| ATOM | 11267 | OE1 | GLU | Y | 197 | −3.266 | 6.892 | 79.087 | 1.00 | 71.32 | Y | O |
| ATOM | 11268 | OE2 | GLU | Y | 197 | −1.214 | 7.260 | 78.349 | 1.00 | 70.18 | Y | O |
| ATOM | 11269 | C | GLU | Y | 197 | −1.074 | 1.958 | 80.078 | 1.00 | 34.77 | Y | C |
| ATOM | 11270 | O | GLU | Y | 197 | −0.932 | 2.466 | 81.191 | 1.00 | 30.79 | Y | O |
| ATOM | 11271 | N | GLY | Y | 198 | −0.806 | 0.685 | 79.810 | 1.00 | 34.09 | Y | N |
| ATOM | 11272 | CA | GLY | Y | 198 | −0.254 | −0.204 | 80.819 | 1.00 | 33.48 | Y | C |
| ATOM | 11273 | C | GLY | Y | 198 | −1.177 | −0.741 | 81.907 | 1.00 | 33.28 | Y | C |
| ATOM | 11274 | O | GLY | Y | 198 | −0.794 | −1.641 | 82.649 | 1.00 | 36.82 | Y | O |
| ATOM | 11275 | N | GLN | Y | 199 | −2.386 | −0.204 | 82.018 | 1.00 | 34.98 | Y | N |
| ATOM | 11276 | CA | GLN | Y | 199 | −3.335 | −0.646 | 83.045 | 1.00 | 34.93 | Y | C |
| ATOM | 11277 | CB | GLN | Y | 199 | −4.019 | 0.570 | 83.686 | 1.00 | 32.28 | Y | C |
| ATOM | 11278 | CG | GLN | Y | 199 | −3.540 | 0.923 | 85.098 | 1.00 | 38.32 | Y | C |
| ATOM | 11279 | CD | GLN | Y | 199 | −2.212 | 1.673 | 85.145 | 1.00 | 39.03 | Y | C |
| ATOM | 11280 | OE1 | GLN | Y | 199 | −1.676 | 1.921 | 86.224 | 1.00 | 38.49 | Y | O |
| ATOM | 11281 | NE2 | GLN | Y | 199 | −1.689 | 2.051 | 83.985 | 1.00 | 30.94 | Y | N |
| ATOM | 11282 | C | GLN | Y | 199 | −4.401 | −1.576 | 82.467 | 1.00 | 35.13 | Y | C |
| ATOM | 11283 | O | GLN | Y | 199 | −5.197 | −1.173 | 81.618 | 1.00 | 37.85 | Y | O |
| ATOM | 11284 | N | SER | Y | 200 | −4.431 | −2.812 | 82.938 | 1.00 | 31.66 | Y | N |
| ATOM | 11285 | CA | SER | Y | 200 | −5.395 | −3.772 | 82.450 | 1.00 | 29.02 | Y | C |
| ATOM | 11286 | CB | SER | Y | 200 | −4.819 | −5.170 | 82.601 | 1.00 | 29.83 | Y | C |
| ATOM | 11287 | OG | SER | Y | 200 | −5.874 | −6.114 | 82.638 | 1.00 | 39.99 | Y | O |
| ATOM | 11288 | C | SER | Y | 200 | −6.793 | −3.686 | 83.102 | 1.00 | 31.23 | Y | C |
| ATOM | 11289 | O | SER | Y | 200 | −6.912 | −3.703 | 84.324 | 1.00 | 30.86 | Y | O |
| ATOM | 11290 | N | VAL | Y | 201 | −7.835 | −3.536 | 82.277 | 1.00 | 30.06 | Y | N |
| ATOM | 11291 | CA | VAL | Y | 201 | −9.228 | −3.488 | 82.750 | 1.00 | 28.76 | Y | C |
| ATOM | 11292 | CB | VAL | Y | 201 | −10.025 | −2.298 | 82.164 | 1.00 | 30.88 | Y | C |
| ATOM | 11293 | CG1 | VAL | Y | 201 | −11.469 | −2.369 | 82.625 | 1.00 | 29.77 | Y | C |
| ATOM | 11294 | CG2 | VAL | Y | 201 | −9.444 | −0.988 | 82.643 | 1.00 | 32.15 | Y | C |
| ATOM | 11295 | C | VAL | Y | 201 | −9.925 | −4.787 | 82.319 | 1.00 | 30.46 | Y | C |
| ATOM | 11296 | O | VAL | Y | 201 | −9.906 | −5.157 | 81.144 | 1.00 | 30.08 | Y | O |
| ATOM | 11297 | N | GLU | Y | 202 | −10.553 | −5.482 | 83.253 | 1.00 | 28.50 | Y | N |
| ATOM | 11298 | CA | GLU | Y | 202 | −11.188 | −6.731 | 82.872 | 1.00 | 37.25 | Y | C |
| ATOM | 11299 | CB | GLU | Y | 202 | −10.288 | −7.923 | 83.219 | 1.00 | 42.92 | Y | C |
| ATOM | 11300 | CG | GLU | Y | 202 | −10.268 | −8.334 | 84.681 | 1.00 | 49.68 | Y | C |
| ATOM | 11301 | CD | GLU | Y | 202 | −9.273 | −9.460 | 84.951 | 1.00 | 53.49 | Y | C |
| ATOM | 11302 | OE1 | GLU | Y | 202 | −8.106 | −9.142 | 85.272 | 1.00 | 52.24 | Y | O |
| ATOM | 11303 | OE2 | GLU | Y | 202 | −9.651 | −10.654 | 84.841 | 1.00 | 51.51 | Y | O |
| ATOM | 11304 | C | GLU | Y | 202 | −12.617 | −6.992 | 83.334 | 1.00 | 33.61 | Y | C |
| ATOM | 11305 | O | GLU | Y | 202 | −13.120 | −6.388 | 84.284 | 1.00 | 39.06 | Y | O |
| ATOM | 11306 | N | LYS | Y | 203 | −13.286 | −7.856 | 82.585 | 1.00 | 27.02 | Y | N |
| ATOM | 11307 | CA | LYS | Y | 203 | −14.654 | −8.273 | 82.884 | 1.00 | 23.23 | Y | C |
| ATOM | 11308 | CB | LYS | Y | 203 | −15.672 | −7.624 | 81.932 | 1.00 | 21.84 | Y | C |
| ATOM | 11309 | CG | LYS | Y | 203 | −16.022 | −6.177 | 82.243 | 1.00 | 15.70 | Y | C |
| ATOM | 11310 | CD | LYS | Y | 203 | −16.766 | −6.045 | 83.560 | 1.00 | 21.64 | Y | C |
| ATOM | 11311 | CE | LYS | Y | 203 | −16.820 | −4.592 | 84.008 | 1.00 | 29.36 | Y | C |
| ATOM | 11312 | NZ | LYS | Y | 203 | −17.686 | −3.755 | 83.143 | 1.00 | 32.52 | Y | N |
| ATOM | 11313 | C | LYS | Y | 203 | −14.702 | −9.782 | 82.734 | 1.00 | 20.03 | Y | C |
| ATOM | 11314 | O | LYS | Y | 203 | −14.080 | −10.355 | 81.836 | 1.00 | 29.38 | Y | O |
| ATOM | 11315 | N | SER | Y | 204 | −15.426 | −10.433 | 83.631 | 1.00 | 33.02 | Y | N |
| ATOM | 11316 | CA | SER | Y | 204 | −15.550 | −11.889 | 83.594 | 1.00 | 32.97 | Y | C |
| ATOM | 11317 | CB | SER | Y | 204 | −14.778 | −12.519 | 84.756 | 1.00 | 37.18 | Y | C |
| ATOM | 11318 | OG | SER | Y | 204 | −13.411 | −12.164 | 84.710 | 1.00 | 44.43 | Y | O |
| ATOM | 11319 | C | SER | Y | 204 | −17.001 | −12.310 | 83.701 | 1.00 | 32.05 | Y | C |
| ATOM | 11320 | O | SER | Y | 204 | −17.884 | −11.490 | 83.989 | 1.00 | 29.71 | Y | O |
| ATOM | 11321 | N | LEU | Y | 205 | −17.243 | −13.593 | 83.455 | 1.00 | 31.01 | Y | N |
| ATOM | 11322 | CA | LEU | Y | 205 | −18.586 | −14.145 | 83.570 | 1.00 | 35.13 | Y | C |
| ATOM | 11323 | CB | LEU | Y | 205 | −19.468 | −13.725 | 82.382 | 1.00 | 34.62 | Y | C |
| ATOM | 11324 | CG | LEU | Y | 205 | −19.070 | −14.035 | 80.932 | 1.00 | 29.17 | Y | C |
| ATOM | 11325 | CD1 | LEU | Y | 205 | −19.236 | −15.490 | 80.653 | 1.00 | 28.67 | Y | C |
| ATOM | 11326 | CD2 | LEU | Y | 205 | −19.948 | −13.248 | 79.971 | 1.00 | 32.08 | Y | C |
| ATOM | 11327 | C | LEU | Y | 205 | −18.525 | −15.655 | 83.713 | 1.00 | 36.99 | Y | C |
| ATOM | 11328 | O | LEU | Y | 205 | −17.475 | −16.268 | 83.498 | 1.00 | 36.10 | Y | O |
| ATOM | 11329 | N | SER | Y | 206 | −19.650 | −16.245 | 84.101 | 1.00 | 45.68 | Y | N |
| ATOM | 11330 | CA | SER | Y | 206 | −19.743 | −17.694 | 84.272 | 1.00 | 49.58 | Y | C |
| ATOM | 11331 | CB | SER | Y | 206 | −19.605 | −18.052 | 85.754 | 1.00 | 50.94 | Y | C |
| ATOM | 11332 | OG | SER | Y | 206 | −18.357 | −17.612 | 86.274 | 1.00 | 48.39 | Y | O |
| ATOM | 11333 | C | SER | Y | 206 | −21.050 | −18.264 | 83.698 | 1.00 | 47.57 | Y | C |
| ATOM | 11334 | O | SER | Y | 206 | −21.085 | −19.396 | 83.196 | 1.00 | 54.36 | Y | O |
| ATOM | 11335 | N | GLU | Z | 1 | −4.547 | 22.435 | 41.209 | 1.00 | 42.57 | Z | N |
| ATOM | 11336 | CA | GLU | Z | 1 | −3.862 | 21.618 | 42.209 | 1.00 | 45.69 | Z | C |
| ATOM | 11337 | CB | GLU | Z | 1 | −4.660 | 21.595 | 43.517 | 1.00 | 50.32 | Z | C |
| ATOM | 11338 | CG | GLU | Z | 1 | −4.835 | 22.972 | 44.163 | 1.00 | 64.80 | Z | C |
| ATOM | 11339 | CD | GLU | Z | 1 | −5.659 | 22.943 | 45.454 | 1.00 | 69.21 | Z | C |
| ATOM | 11340 | OE1 | GLU | Z | 1 | −6.060 | 21.838 | 45.898 | 1.00 | 74.37 | Z | O |
| ATOM | 11341 | OE2 | GLU | Z | 1 | −5.905 | 24.034 | 46.024 | 1.00 | 69.82 | Z | O |
| ATOM | 11342 | C | GLU | Z | 1 | −3.659 | 20.189 | 41.719 | 1.00 | 40.44 | Z | C |
| ATOM | 11343 | O | GLU | Z | 1 | −4.413 | 19.705 | 40.867 | 1.00 | 35.39 | Z | O |
| ATOM | 11344 | N | VAL | Z | 2 | −2.636 | 19.520 | 42.250 | 1.00 | 34.72 | Z | N |
| ATOM | 11345 | CA | VAL | Z | 2 | −2.365 | 18.140 | 41.869 | 1.00 | 32.59 | Z | C |
| ATOM | 11346 | CB | VAL | Z | 2 | −1.009 | 17.639 | 42.387 | 1.00 | 34.77 | Z | C |
| ATOM | 11347 | CG1 | VAL | Z | 2 | −0.840 | 16.139 | 42.084 | 1.00 | 26.57 | Z | C |
| ATOM | 11348 | CG2 | VAL | Z | 2 | 0.128 | 18.434 | 41.721 | 1.00 | 32.16 | Z | C |
| ATOM | 11349 | C | VAL | Z | 2 | −3.487 | 17.288 | 42.421 | 1.00 | 38.33 | Z | C |
| ATOM | 11350 | O | VAL | Z | 2 | −3.933 | 17.496 | 43.553 | 1.00 | 44.67 | Z | O |
| ATOM | 11351 | N | GLN | Z | 3 | −3.947 | 16.339 | 41.610 | 1.00 | 38.63 | Z | N |
| ATOM | 11352 | CA | GLN | Z | 3 | −5.057 | 15.486 | 41.989 | 1.00 | 39.40 | Z | C |
| ATOM | 11353 | CB | GLN | Z | 3 | −6.344 | 16.153 | 41.505 | 1.00 | 45.20 | Z | C |
| ATOM | 11354 | CG | GLN | Z | 3 | −7.582 | 15.879 | 42.322 | 1.00 | 53.03 | Z | C |
| ATOM | 11355 | CD | GLN | Z | 3 | −8.697 | 16.887 | 42.045 | 1.00 | 55.19 | Z | C |
| ATOM | 11356 | OE1 | GLN | Z | 3 | −9.344 | 17.386 | 42.967 | 1.00 | 55.77 | Z | O |
| ATOM | 11357 | NE2 | GLN | Z | 3 | −8.918 | 17.192 | 40.771 | 1.00 | 57.72 | Z | N |
| ATOM | 11358 | C | GLN | Z | 3 | −4.916 | 14.115 | 41.349 | 1.00 | 38.05 | Z | C |
| ATOM | 11359 | O | GLN | Z | 3 | −4.560 | 14.010 | 40.179 | 1.00 | 37.99 | Z | O |
| ATOM | 11360 | N | LEU | Z | 4 | −5.157 | 13.071 | 42.140 | 1.00 | 33.40 | Z | N |
| ATOM | 11361 | CA | LEU | Z | 4 | −5.096 | 11.689 | 41.670 | 1.00 | 29.25 | Z | C |
| ATOM | 11362 | CB | LEU | Z | 4 | −3.954 | 10.941 | 42.363 | 1.00 | 29.02 | Z | C |
| ATOM | 11363 | CG | LEU | Z | 4 | −2.556 | 10.767 | 41.746 | 1.00 | 30.67 | Z | C |
| ATOM | 11364 | CD1 | LEU | Z | 4 | −1.916 | 12.093 | 41.410 | 1.00 | 29.21 | Z | C |
| ATOM | 11365 | CD2 | LEU | Z | 4 | −1.673 | 10.003 | 42.731 | 1.00 | 29.50 | Z | C |
| ATOM | 11366 | C | LEU | Z | 4 | −6.451 | 11.012 | 41.995 | 1.00 | 34.22 | Z | C |
| ATOM | 11367 | O | LEU | Z | 4 | −6.667 | 10.580 | 43.129 | 1.00 | 33.96 | Z | O |
| ATOM | 11368 | N | GLN | Z | 5 | −7.366 | 10.956 | 41.017 | 1.00 | 32.00 | Z | N |
| ATOM | 11369 | CA | GLN | Z | 5 | −8.694 | 10.350 | 41.211 | 1.00 | 31.01 | Z | C |
| ATOM | 11370 | CB | GLN | Z | 5 | −9.755 | 11.098 | 40.407 | 1.00 | 34.54 | Z | C |
| ATOM | 11371 | CG | GLN | Z | 5 | −9.965 | 12.556 | 40.789 | 1.00 | 47.29 | Z | C |
| ATOM | 11372 | CD | GLN | Z | 5 | −10.753 | 13.331 | 39.731 | 1.00 | 52.80 | Z | C |
| ATOM | 11373 | OE1 | GLN | Z | 5 | −11.235 | 14.439 | 39.988 | 1.00 | 57.38 | Z | O |
| ATOM | 11374 | NE2 | GLN | Z | 5 | −10.874 | 12.754 | 38.531 | 1.00 | 53.27 | Z | N |
| ATOM | 11375 | C | GLN | Z | 5 | −8.743 | 8.873 | 40.831 | 1.00 | 28.09 | Z | C |
| ATOM | 11376 | O | GLN | Z | 5 | −8.624 | 8.510 | 39.654 | 1.00 | 27.36 | Z | O |
| ATOM | 11377 | N | GLN | Z | 6 | −8.934 | 8.019 | 41.828 | 1.00 | 27.24 | Z | N |
| ATOM | 11378 | CA | GLN | Z | 6 | −8.994 | 6.583 | 41.584 | 1.00 | 23.88 | Z | C |
| ATOM | 11379 | CB | GLN | Z | 6 | −8.401 | 5.801 | 42.761 | 1.00 | 24.71 | Z | C |
| ATOM | 11380 | CG | GLN | Z | 6 | −6.892 | 6.021 | 42.919 | 1.00 | 21.12 | Z | C |
| ATOM | 11381 | CD | GLN | Z | 6 | −6.245 | 5.193 | 44.019 | 1.00 | 28.56 | Z | C |
| ATOM | 11382 | OE1 | GLN | Z | 6 | −5.418 | 5.706 | 44.792 | 1.00 | 31.63 | Z | O |
| ATOM | 11383 | NE2 | GLN | Z | 6 | −6.566 | 3.899 | 44.063 | 1.00 | 24.94 | Z | N |
| ATOM | 11384 | C | GLN | Z | 6 | −10.412 | 6.144 | 41.274 | 1.00 | 24.96 | Z | C |
| ATOM | 11385 | O | GLN | Z | 6 | −11.367 | 6.914 | 41.421 | 1.00 | 26.19 | Z | O |
| ATOM | 11386 | N | SER | Z | 7 | −10.543 | 4.910 | 40.806 | 1.00 | 30.32 | Z | N |
| ATOM | 11387 | CA | SER | Z | 7 | −11.845 | 4.373 | 40.425 | 1.00 | 24.89 | Z | C |
| ATOM | 11388 | CB | SER | Z | 7 | −11.690 | 3.384 | 39.272 | 1.00 | 20.07 | Z | C |
| ATOM | 11389 | OG | SER | Z | 7 | −10.752 | 2.377 | 39.599 | 1.00 | 28.61 | Z | O |
| ATOM | 11390 | C | SER | Z | 7 | −12.603 | 3.742 | 41.583 | 1.00 | 25.90 | Z | C |
| ATOM | 11391 | O | SER | Z | 7 | −12.005 | 3.285 | 42.567 | 1.00 | 28.08 | Z | O |
| ATOM | 11392 | N | GLY | Z | 8 | −13.927 | 3.726 | 41.432 | 1.00 | 23.90 | Z | N |
| ATOM | 11393 | CA | GLY | Z | 8 | −14.828 | 3.188 | 42.426 | 1.00 | 19.26 | Z | C |
| ATOM | 11394 | C | GLY | Z | 8 | −14.502 | 1.803 | 42.927 | 1.00 | 23.01 | Z | C |
| ATOM | 11395 | O | GLY | Z | 8 | −13.740 | 1.075 | 42.301 | 1.00 | 25.33 | Z | O |
| ATOM | 11396 | N | PRO | Z | 9 | −15.154 | 1.381 | 44.021 | 1.00 | 28.40 | Z | N |
| ATOM | 11397 | CD | PRO | Z | 9 | −16.274 | 2.114 | 44.643 | 1.00 | 20.18 | Z | C |
| ATOM | 11398 | CA | PRO | Z | 9 | −14.960 | 0.073 | 44.657 | 1.00 | 30.80 | Z | C |
| ATOM | 11399 | CB | PRO | Z | 9 | −15.869 | 0.159 | 45.891 | 1.00 | 31.47 | Z | C |
| ATOM | 11400 | CG | PRO | Z | 9 | −16.987 | 1.040 | 45.415 | 1.00 | 26.10 | Z | C |
| ATOM | 11401 | C | PRO | Z | 9 | −15.325 | −1.087 | 43.747 | 1.00 | 32.21 | Z | C |
| ATOM | 11402 | O | PRO | Z | 9 | −16.073 | −0.916 | 42.786 | 1.00 | 31.94 | Z | O |
| ATOM | 11403 | N | GLU | Z | 10 | −14.806 | −2.267 | 44.066 | 1.00 | 31.92 | Z | N |
| ATOM | 11404 | CA | GLU | Z | 10 | −15.059 | −3.451 | 43.260 | 1.00 | 36.75 | Z | C |
| ATOM | 11405 | CB | GLU | Z | 10 | −13.862 | −3.689 | 42.330 | 1.00 | 40.26 | Z | C |
| ATOM | 11406 | CG | GLU | Z | 10 | −14.218 | −4.145 | 40.923 | 1.00 | 53.72 | Z | C |
| ATOM | 11407 | CD | GLU | Z | 10 | −14.424 | −2.995 | 39.940 | 1.00 | 55.35 | Z | C |
| ATOM | 11408 | OE1 | GLU | Z | 10 | −15.585 | −2.726 | 39.561 | 1.00 | 64.13 | Z | O |
| ATOM | 11409 | OE2 | GLU | Z | 10 | −13.421 | −2.378 | 39.517 | 1.00 | 60.44 | Z | O |
| ATOM | 11410 | C | GLU | Z | 10 | −15.294 | −4.687 | 44.145 | 1.00 | 38.79 | Z | C |
| ATOM | 11411 | O | GLU | Z | 10 | −14.605 | −4.882 | 45.147 | 1.00 | 38.32 | Z | O |
| ATOM | 11412 | N | LEU | Z | 11 | −16.311 | −5.479 | 43.798 | 1.00 | 38.20 | Z | N |
| ATOM | 11413 | CA | LEU | Z | 11 | −16.651 | −6.711 | 44.510 | 1.00 | 31.37 | Z | C |
| ATOM | 11414 | CB | LEU | Z | 11 | −18.015 | −6.583 | 45.188 | 1.00 | 36.59 | Z | C |
| ATOM | 11415 | CG | LEU | Z | 11 | −18.692 | −7.887 | 45.632 | 1.00 | 39.46 | Z | C |
| ATOM | 11416 | CD1 | LEU | Z | 11 | −17.772 | −8.724 | 46.525 | 1.00 | 36.91 | Z | C |
| ATOM | 11417 | CD2 | LEU | Z | 11 | −19.997 | −7.558 | 46.339 | 1.00 | 42.03 | Z | C |
| ATOM | 11418 | C | LEU | Z | 11 | −16.705 | −7.799 | 43.456 | 1.00 | 32.93 | Z | C |
| ATOM | 11419 | O | LEU | Z | 11 | −17.534 | −7.738 | 42.551 | 1.00 | 33.67 | Z | O |
| ATOM | 11420 | N | VAL | Z | 12 | −15.832 | −8.796 | 43.577 | 1.00 | 31.66 | Z | N |
| ATOM | 11421 | CA | VAL | Z | 12 | −15.763 | −9.870 | 42.597 | 1.00 | 30.70 | Z | C |
| ATOM | 11422 | CB | VAL | Z | 12 | −14.701 | −9.566 | 41.499 | 1.00 | 37.71 | Z | C |
| ATOM | 11423 | CG1 | VAL | Z | 12 | −15.063 | −8.295 | 40.721 | 1.00 | 29.19 | Z | C |
| ATOM | 11424 | CG2 | VAL | Z | 12 | −13.316 | −9.448 | 42.131 | 1.00 | 26.02 | Z | C |
| ATOM | 11425 | C | VAL | Z | 12 | −15.419 | −11.231 | 43.188 | 1.00 | 33.94 | Z | C |
| ATOM | 11426 | O | VAL | Z | 12 | −14.902 | −11.330 | 44.300 | 1.00 | 40.19 | Z | O |
| ATOM | 11427 | N | LYS | Z | 13 | −15.594 | −12.258 | 42.361 | 1.00 | 31.26 | Z | N |
| ATOM | 11428 | CA | LYS | Z | 13 | −15.364 | −13.646 | 42.731 | 1.00 | 30.16 | Z | C |
| ATOM | 11429 | CB | LYS | Z | 13 | −16.333 | −14.547 | 41.936 | 1.00 | 32.44 | Z | C |
| ATOM | 11430 | CG | LYS | Z | 13 | −17.836 | −14.275 | 42.184 | 1.00 | 27.64 | Z | C |
| ATOM | 11431 | CD | LYS | Z | 13 | −18.721 | −15.253 | 41.426 | 0.00 | 29.21 | Z | C |
| ATOM | 11432 | CE | LYS | Z | 13 | −18.594 | −15.077 | 39.922 | 0.00 | 28.46 | Z | C |
| ATOM | 11433 | NZ | LYS | Z | 13 | −19.493 | −16.006 | 39.184 | 0.00 | 28.93 | Z | N |
| ATOM | 11434 | C | LYS | Z | 13 | −13.928 | −14.099 | 42.486 | 1.00 | 31.95 | Z | C |
| ATOM | 11435 | O | LYS | Z | 13 | −13.240 | −13.562 | 41.619 | 1.00 | 39.66 | Z | O |
| ATOM | 11436 | N | PRO | Z | 14 | −13.459 | −15.106 | 43.244 | 1.00 | 31.10 | Z | N |
| ATOM | 11437 | CD | PRO | Z | 14 | −14.202 | −15.823 | 44.296 | 1.00 | 33.54 | Z | C |
| ATOM | 11438 | CA | PRO | Z | 14 | −12.103 | −15.646 | 43.110 | 1.00 | 34.95 | Z | C |
| ATOM | 11439 | CB | PRO | Z | 14 | −12.126 | −16.857 | 44.040 | 1.00 | 33.03 | Z | C |
| ATOM | 11440 | CG | PRO | Z | 14 | −13.102 | −16.459 | 45.092 | 1.00 | 32.54 | Z | C |
| ATOM | 11441 | C | PRO | Z | 14 | −11.849 | −16.100 | 41.678 | 1.00 | 42.24 | Z | C |
| ATOM | 11442 | O | PRO | Z | 14 | −12.770 | −16.557 | 40.985 | 1.00 | 45.65 | Z | O |
| ATOM | 11443 | N | GLY | Z | 15 | −10.597 | −15.986 | 41.244 | 1.00 | 42.62 | Z | N |
| ATOM | 11444 | CA | GLY | Z | 15 | −10.249 | −16.392 | 39.897 | 1.00 | 41.62 | Z | C |
| ATOM | 11445 | C | GLY | Z | 15 | −10.550 | −15.324 | 38.867 | 1.00 | 41.87 | Z | C |
| ATOM | 11446 | O | GLY | Z | 15 | −10.195 | −15.470 | 37.697 | 1.00 | 44.36 | Z | O |
| ATOM | 11447 | N | ALA | Z | 16 | −11.224 | −14.259 | 39.290 | 1.00 | 39.08 | Z | N |
| ATOM | 11448 | CA | ALA | Z | 16 | −11.537 | −13.153 | 38.393 | 1.00 | 35.65 | Z | C |
| ATOM | 11449 | CB | ALA | Z | 16 | −12.777 | −12.407 | 38.882 | 1.00 | 31.87 | Z | C |
| ATOM | 11450 | C | ALA | Z | 16 | −10.343 | −12.201 | 38.332 | 1.00 | 36.48 | Z | C |
| ATOM | 11451 | O | ALA | Z | 16 | −9.288 | −12.450 | 38.934 | 1.00 | 37.71 | Z | O |
| ATOM | 11452 | N | SER | Z | 17 | −10.514 | −11.101 | 37.613 | 1.00 | 36.46 | Z | N |
| ATOM | 11453 | CA | SER | Z | 17 | −9.451 | −10.127 | 37.506 | 1.00 | 41.81 | Z | C |
| ATOM | 11454 | CB | SER | Z | 17 | −8.637 | −10.354 | 36.226 | 1.00 | 41.39 | Z | C |
| ATOM | 11455 | OG | SER | Z | 17 | −9.271 | −9.776 | 35.108 | 1.00 | 51.39 | Z | O |
| ATOM | 11456 | C | SER | Z | 17 | −10.078 | −8.743 | 37.535 | 1.00 | 41.46 | Z | C |
| ATOM | 11457 | O | SER | Z | 17 | −11.245 | −8.582 | 37.197 | 1.00 | 42.60 | Z | O |
| ATOM | 11458 | N | VAL | Z | 18 | −9.322 | −7.757 | 38.000 | 1.00 | 40.20 | Z | N |
| ATOM | 11459 | CA | VAL | Z | 18 | −9.835 | −6.397 | 38.073 | 1.00 | 37.63 | Z | C |
| ATOM | 11460 | CB | VAL | Z | 18 | −10.310 | −6.043 | 39.528 | 1.00 | 40.98 | Z | C |
| ATOM | 11461 | CG1 | VAL | Z | 18 | −9.142 | −5.958 | 40.485 | 1.00 | 36.82 | Z | C |
| ATOM | 11462 | CG2 | VAL | Z | 18 | −11.094 | −4.758 | 39.536 | 1.00 | 38.94 | Z | C |
| ATOM | 11463 | C | VAL | Z | 18 | −8.796 | −5.392 | 37.579 | 1.00 | 40.58 | Z | C |
| ATOM | 11464 | O | VAL | Z | 18 | −7.582 | −5.596 | 37.724 | 1.00 | 42.50 | Z | O |
| ATOM | 11465 | N | LYS | Z | 19 | −9.281 | −4.328 | 36.954 | 1.00 | 34.57 | Z | N |
| ATOM | 11466 | CA | LYS | Z | 19 | −8.419 | −3.296 | 36.438 | 1.00 | 29.68 | Z | C |
| ATOM | 11467 | CB | LYS | Z | 19 | −8.534 | −3.240 | 34.916 | 1.00 | 32.44 | Z | C |
| ATOM | 11468 | CG | LYS | Z | 19 | −7.383 | −2.513 | 34.244 | 1.00 | 35.99 | Z | C |
| ATOM | 11469 | CD | LYS | Z | 19 | −7.531 | −2.456 | 32.741 | 1.00 | 36.63 | Z | C |
| ATOM | 11470 | CE | LYS | Z | 19 | −6.256 | −1.934 | 32.099 | 1.00 | 35.94 | Z | C |
| ATOM | 11471 | NZ | LYS | Z | 19 | −6.270 | −2.213 | 30.645 | 1.00 | 39.24 | Z | N |
| ATOM | 11472 | C | LYS | Z | 19 | −8.813 | −1.962 | 37.053 | 1.00 | 28.87 | Z | C |
| ATOM | 11473 | O | LYS | Z | 19 | −9.814 | −1.370 | 36.665 | 1.00 | 34.61 | Z | O |
| ATOM | 11474 | N | ILE | Z | 20 | −8.022 | −1.494 | 38.016 | 1.00 | 31.89 | Z | N |
| ATOM | 11475 | CA | ILE | Z | 20 | −8.270 | −0.216 | 38.705 | 1.00 | 28.82 | Z | C |
| ATOM | 11476 | CB | ILE | Z | 20 | −7.716 | −0.278 | 40.135 | 1.00 | 25.70 | Z | C |
| ATOM | 11477 | CG2 | ILE | Z | 20 | −7.929 | 1.028 | 40.850 | 1.00 | 28.15 | Z | C |
| ATOM | 11478 | CG1 | ILE | Z | 20 | −8.367 | −1.418 | 40.901 | 1.00 | 26.71 | Z | C |
| ATOM | 11479 | CD1 | ILE | Z | 20 | −7.507 | −1.923 | 42.047 | 1.00 | 30.00 | Z | C |
| ATOM | 11480 | C | ILE | Z | 20 | −7.564 | 0.939 | 37.982 | 1.00 | 26.22 | Z | C |
| ATOM | 11481 | O | ILE | Z | 20 | −6.455 | 0.766 | 37.497 | 1.00 | 31.85 | Z | O |
| ATOM | 11482 | N | SER | Z | 21 | −8.184 | 2.115 | 37.928 | 1.00 | 19.13 | Z | N |
| ATOM | 11483 | CA | SER | Z | 21 | −7.548 | 3.250 | 37.273 | 1.00 | 21.86 | Z | C |
| ATOM | 11484 | CB | SER | Z | 21 | −8.397 | 3.744 | 36.094 | 1.00 | 22.16 | Z | C |
| ATOM | 11485 | OG | SER | Z | 21 | −9.549 | 4.423 | 36.538 | 1.00 | 30.34 | Z | O |
| ATOM | 11486 | C | SER | Z | 21 | −7.248 | 4.419 | 38.225 | 1.00 | 22.68 | Z | C |
| ATOM | 11487 | O | SER | Z | 21 | −7.880 | 4.550 | 39.271 | 1.00 | 27.89 | Z | O |
| ATOM | 11488 | N | CYS | Z | 22 | −6.262 | 5.242 | 37.853 | 1.00 | 21.56 | Z | N |
| ATOM | 11489 | CA | CYS | Z | 22 | −5.844 | 6.431 | 38.611 | 1.00 | 23.83 | Z | C |
| ATOM | 11490 | C | CYS | Z | 22 | −5.700 | 7.519 | 37.572 | 1.00 | 21.76 | Z | C |
| ATOM | 11491 | O | CYS | Z | 22 | −4.832 | 7.439 | 36.705 | 1.00 | 31.55 | Z | O |
| ATOM | 11492 | CB | CYS | Z | 22 | −4.474 | 6.225 | 39.286 | 1.00 | 25.37 | Z | C |
| ATOM | 11493 | SG | CYS | Z | 22 | −3.988 | 7.470 | 40.539 | 0.50 | 23.30 | Z | S |
| ATOM | 11494 | N | LYS | Z | 23 | −6.590 | 8.499 | 37.627 | 1.00 | 19.83 | Z | N |
| ATOM | 11495 | CA | LYS | Z | 23 | −6.584 | 9.613 | 36.702 | 1.00 | 20.07 | Z | C |
| ATOM | 11496 | CB | LYS | Z | 23 | −8.031 | 9.990 | 36.348 | 1.00 | 19.57 | Z | C |
| ATOM | 11497 | CG | LYS | Z | 23 | −8.227 | 11.324 | 35.626 | 1.00 | 22.42 | Z | C |
| ATOM | 11498 | CD | LYS | Z | 23 | −7.533 | 11.351 | 34.263 | 1.00 | 30.08 | Z | C |
| ATOM | 11499 | CE | LYS | Z | 23 | −7.489 | 12.776 | 33.688 | 1.00 | 34.78 | Z | C |
| ATOM | 11500 | NZ | LYS | Z | 23 | −6.839 | 12.878 | 32.337 | 1.00 | 28.17 | Z | N |
| ATOM | 11501 | C | LYS | Z | 23 | −5.823 | 10.804 | 37.306 | 1.00 | 26.17 | Z | C |
| ATOM | 11502 | O | LYS | Z | 23 | −6.293 | 11.435 | 38.259 | 1.00 | 26.87 | Z | O |
| ATOM | 11503 | N | ALA | Z | 24 | −4.642 | 11.093 | 36.749 | 1.00 | 29.45 | Z | N |
| ATOM | 11504 | CA | ALA | Z | 24 | −3.805 | 12.203 | 37.214 | 1.00 | 27.03 | Z | C |
| ATOM | 11505 | CB | ALA | Z | 24 | −2.354 | 11.882 | 36.998 | 1.00 | 24.46 | Z | C |
| ATOM | 11506 | C | ALA | Z | 24 | −4.149 | 13.515 | 36.518 | 1.00 | 29.63 | Z | C |
| ATOM | 11507 | O | ALA | Z | 24 | −4.418 | 13.532 | 35.323 | 1.00 | 36.04 | Z | O |
| ATOM | 11508 | N | SER | Z | 25 | −4.145 | 14.613 | 37.269 | 1.00 | 28.18 | Z | N |
| ATOM | 11509 | CA | SER | Z | 25 | −4.439 | 15.923 | 36.705 | 1.00 | 26.42 | Z | C |
| ATOM | 11510 | CB | SER | Z | 25 | −5.946 | 16.153 | 36.639 | 1.00 | 25.66 | Z | C |
| ATOM | 11511 | OG | SER | Z | 25 | −6.474 | 16.409 | 37.925 | 1.00 | 28.72 | Z | O |
| ATOM | 11512 | C | SER | Z | 25 | −3.775 | 17.019 | 37.543 | 1.00 | 28.75 | Z | C |
| ATOM | 11513 | O | SER | Z | 25 | −3.264 | 16.755 | 38.631 | 1.00 | 34.14 | Z | O |
| ATOM | 11514 | N | GLY | Z | 26 | −3.769 | 18.243 | 37.028 | 1.00 | 29.65 | Z | N |
| ATOM | 11515 | CA | GLY | Z | 26 | −3.176 | 19.354 | 37.758 | 1.00 | 28.70 | Z | C |
| ATOM | 11516 | C | GLY | Z | 26 | −1.662 | 19.483 | 37.652 | 1.00 | 32.32 | Z | C |
| ATOM | 11517 | O | GLY | Z | 26 | −1.069 | 20.336 | 38.323 | 1.00 | 31.30 | Z | O |
| ATOM | 11518 | N | TYR | Z | 27 | −1.045 | 18.638 | 36.825 | 1.00 | 23.51 | Z | N |
| ATOM | 11519 | CA | TYR | Z | 27 | 0.399 | 18.648 | 36.619 | 1.00 | 27.64 | Z | C |
| ATOM | 11520 | CB | TYR | Z | 27 | 1.116 | 18.048 | 37.837 | 1.00 | 16.71 | Z | C |
| ATOM | 11521 | CG | TYR | Z | 27 | 1.025 | 16.543 | 37.938 | 1.00 | 17.24 | Z | C |
| ATOM | 11522 | CD1 | TYR | Z | 27 | −0.076 | 15.934 | 38.524 | 1.00 | 18.94 | Z | C |
| ATOM | 11523 | CE1 | TYR | Z | 27 | −0.162 | 14.540 | 38.639 | 1.00 | 14.27 | Z | C |
| ATOM | 11524 | CD2 | TYR | Z | 27 | 2.045 | 15.728 | 37.463 | 1.00 | 15.40 | Z | C |
| ATOM | 11525 | CE2 | TYR | Z | 27 | 1.970 | 14.331 | 37.573 | 1.00 | 17.68 | Z | C |
| ATOM | 11526 | CZ | TYR | Z | 27 | 0.855 | 13.746 | 38.167 | 1.00 | 16.14 | Z | C |
| ATOM | 11527 | OH | TYR | Z | 27 | 0.752 | 12.369 | 38.291 | 1.00 | 16.72 | Z | O |
| ATOM | 11528 | C | TYR | Z | 27 | 0.800 | 17.862 | 35.358 | 1.00 | 26.18 | Z | C |
| ATOM | 11529 | O | TYR | Z | 27 | −0.004 | 17.119 | 34.801 | 1.00 | 24.70 | Z | O |
| ATOM | 11530 | N | SER | Z | 28 | 2.069 | 17.975 | 34.974 | 1.00 | 28.15 | Z | N |
| ATOM | 11531 | CA | SER | Z | 28 | 2.604 | 17.274 | 33.802 | 1.00 | 28.38 | Z | C |
| ATOM | 11532 | CB | SER | Z | 28 | 3.917 | 17.921 | 33.357 | 1.00 | 24.43 | Z | C |
| ATOM | 11533 | OG | SER | Z | 28 | 4.459 | 17.262 | 32.228 | 1.00 | 35.51 | Z | O |
| ATOM | 11534 | C | SER | Z | 28 | 2.814 | 15.784 | 34.093 | 1.00 | 29.83 | Z | C |
| ATOM | 11535 | O | SER | Z | 28 | 3.761 | 15.389 | 34.766 | 1.00 | 34.00 | Z | O |
| ATOM | 11536 | N | PHE | Z | 29 | 1.940 | 14.969 | 33.525 | 1.00 | 24.00 | Z | N |
| ATOM | 11537 | CA | PHE | Z | 29 | 1.933 | 13.519 | 33.685 | 1.00 | 24.34 | Z | C |
| ATOM | 11538 | CB | PHE | Z | 29 | 1.008 | 12.933 | 32.618 | 1.00 | 23.81 | Z | C |
| ATOM | 11539 | CG | PHE | Z | 29 | 0.659 | 11.499 | 32.836 | 1.00 | 19.26 | Z | C |
| ATOM | 11540 | CD1 | PHE | Z | 29 | 0.106 | 11.088 | 34.034 | 1.00 | 16.55 | Z | C |
| ATOM | 11541 | CD2 | PHE | Z | 29 | 0.792 | 10.577 | 31.806 | 1.00 | 24.64 | Z | C |
| ATOM | 11542 | CE1 | PHE | Z | 29 | −0.323 | 9.789 | 34.209 | 1.00 | 14.33 | Z | C |
| ATOM | 11543 | CE2 | PHE | Z | 29 | 0.361 | 9.269 | 31.974 | 1.00 | 19.70 | Z | C |
| ATOM | 11544 | CZ | PHE | Z | 29 | −0.200 | 8.880 | 33.183 | 1.00 | 16.89 | Z | C |
| ATOM | 11545 | C | PHE | Z | 29 | 3.249 | 12.741 | 33.627 | 1.00 | 23.55 | Z | C |
| ATOM | 11546 | O | PHE | Z | 29 | 3.518 | 11.885 | 34.464 | 1.00 | 25.99 | Z | O |
| ATOM | 11547 | N | THR | Z | 30 | 3.984 | 12.936 | 32.545 | 1.00 | 20.09 | Z | N |
| ATOM | 11548 | CA | THR | Z | 30 | 5.227 | 12.230 | 32.324 | 1.00 | 18.67 | Z | C |
| ATOM | 11549 | CB | THR | Z | 30 | 5.654 | 12.410 | 30.860 | 1.00 | 23.68 | Z | C |
| ATOM | 11550 | OG1 | THR | Z | 30 | 5.902 | 13.795 | 30.605 | 1.00 | 31.53 | Z | O |
| ATOM | 11551 | CG2 | THR | Z | 30 | 4.547 | 11.967 | 29.916 | 1.00 | 27.32 | Z | C |
| ATOM | 11552 | C | THR | Z | 30 | 6.394 | 12.641 | 33.246 | 1.00 | 21.71 | Z | C |
| ATOM | 11553 | O | THR | Z | 30 | 7.441 | 12.004 | 33.223 | 1.00 | 23.59 | Z | O |
| ATOM | 11554 | N | GLY | Z | 31 | 6.211 | 13.669 | 34.072 | 1.00 | 17.90 | Z | N |
| ATOM | 11555 | CA | GLY | Z | 31 | 7.290 | 14.116 | 34.948 | 1.00 | 21.89 | Z | C |
| ATOM | 11556 | C | GLY | Z | 31 | 7.442 | 13.592 | 36.377 | 1.00 | 27.84 | Z | C |
| ATOM | 11557 | O | GLY | Z | 31 | 8.285 | 14.101 | 37.103 | 1.00 | 29.51 | Z | O |
| ATOM | 11558 | N | HIS | Z | 32 | 6.620 | 12.629 | 36.806 | 1.00 | 26.79 | Z | N |
| ATOM | 11559 | CA | HIS | Z | 32 | 6.718 | 12.042 | 38.152 | 1.00 | 24.11 | Z | C |
| ATOM | 11560 | CB | HIS | Z | 32 | 5.679 | 12.612 | 39.113 | 1.00 | 21.96 | Z | C |
| ATOM | 11561 | CG | HIS | Z | 32 | 6.019 | 13.951 | 39.678 | 1.00 | 11.48 | Z | C |
| ATOM | 11562 | CD2 | HIS | Z | 32 | 6.279 | 14.339 | 40.949 | 1.00 | 15.42 | Z | C |
| ATOM | 11563 | ND1 | HIS | Z | 32 | 6.026 | 15.098 | 38.920 | 1.00 | 12.69 | Z | N |
| ATOM | 11564 | CE1 | HIS | Z | 32 | 6.272 | 16.138 | 39.696 | 1.00 | 17.20 | Z | C |
| ATOM | 11565 | NE2 | HIS | Z | 32 | 6.429 | 15.704 | 40.935 | 1.00 | 17.22 | Z | N |
| ATOM | 11566 | C | HIS | Z | 32 | 6.431 | 10.556 | 38.028 | 1.00 | 26.73 | Z | C |
| ATOM | 11567 | O | HIS | Z | 32 | 5.729 | 10.148 | 37.116 | 1.00 | 26.91 | Z | O |
| ATOM | 11568 | N | LEU | Z | 33 | 7.002 | 9.750 | 38.918 | 1.00 | 20.49 | Z | N |
| ATOM | 11569 | CA | LEU | Z | 33 | 6.756 | 8.323 | 38.895 | 1.00 | 21.23 | Z | C |
| ATOM | 11570 | CB | LEU | Z | 33 | 7.818 | 7.584 | 39.718 | 1.00 | 18.00 | Z | C |
| ATOM | 11571 | CG | LEU | Z | 33 | 9.222 | 7.483 | 39.126 | 1.00 | 19.93 | Z | C |
| ATOM | 11572 | CD1 | LEU | Z | 33 | 10.088 | 6.543 | 39.957 | 1.00 | 8.74 | Z | C |
| ATOM | 11573 | CD2 | LEU | Z | 33 | 9.112 | 6.942 | 37.696 | 1.00 | 19.64 | Z | C |
| ATOM | 11574 | C | LEU | Z | 33 | 5.374 | 8.087 | 39.504 | 1.00 | 20.49 | Z | C |
| ATOM | 11575 | O | LEU | Z | 33 | 4.809 | 8.987 | 40.125 | 1.00 | 21.31 | Z | O |
| ATOM | 11576 | N | LEU | Z | 34 | 4.776 | 6.935 | 39.220 | 1.00 | 20.06 | Z | N |
| ATOM | 11577 | CA | LEU | Z | 34 | 3.478 | 6.596 | 39.811 | 1.00 | 20.43 | Z | C |
| ATOM | 11578 | CB | LEU | Z | 34 | 2.347 | 6.603 | 38.797 | 1.00 | 19.27 | Z | C |
| ATOM | 11579 | CG | LEU | Z | 34 | 1.818 | 7.999 | 38.467 | 1.00 | 26.66 | Z | C |
| ATOM | 11580 | CD1 | LEU | Z | 34 | 0.882 | 7.975 | 37.263 | 1.00 | 23.36 | Z | C |
| ATOM | 11581 | CD2 | LEU | Z | 34 | 1.120 | 8.568 | 39.691 | 1.00 | 27.57 | Z | C |
| ATOM | 11582 | C | LEU | Z | 34 | 3.676 | 5.230 | 40.391 | 1.00 | 21.84 | Z | C |
| ATOM | 11583 | O | LEU | Z | 34 | 4.189 | 4.340 | 39.710 | 1.00 | 25.90 | Z | O |
| ATOM | 11584 | N | ASN | Z | 35 | 3.423 | 5.126 | 41.693 | 1.00 | 19.91 | Z | N |
| ATOM | 11585 | CA | ASN | Z | 35 | 3.578 | 3.887 | 42.438 | 1.00 | 17.41 | Z | C |
| ATOM | 11586 | CB | ASN | Z | 35 | 4.279 | 4.167 | 43.767 | 1.00 | 18.17 | Z | C |
| ATOM | 11587 | CG | ASN | Z | 35 | 5.712 | 4.648 | 43.596 | 1.00 | 22.48 | Z | C |
| ATOM | 11588 | OD1 | ASN | Z | 35 | 6.649 | 3.868 | 43.689 | 1.00 | 24.64 | Z | O |
| ATOM | 11589 | ND2 | ASN | Z | 35 | 5.884 | 5.937 | 43.378 | 1.00 | 14.06 | Z | N |
| ATOM | 11590 | C | ASN | Z | 35 | 2.210 | 3.314 | 42.753 | 1.00 | 22.21 | Z | C |
| ATOM | 11591 | O | ASN | Z | 35 | 1.223 | 4.045 | 42.785 | 1.00 | 22.25 | Z | O |
| ATOM | 11592 | N | TRP | Z | 36 | 2.167 | 2.012 | 43.019 | 1.00 | 23.60 | Z | N |
| ATOM | 11593 | CA | TRP | Z | 36 | 0.937 | 1.326 | 43.375 | 1.00 | 18.27 | Z | C |
| ATOM | 11594 | CB | TRP | Z | 36 | 0.544 | 0.286 | 42.321 | 1.00 | 20.65 | Z | C |
| ATOM | 11595 | CG | TRP | Z | 36 | −0.230 | 0.910 | 41.234 | 1.00 | 21.22 | Z | C |
| ATOM | 11596 | CD2 | TRP | Z | 36 | −1.570 | 1.393 | 41.314 | 1.00 | 25.07 | Z | C |
| ATOM | 11597 | CE2 | TRP | Z | 36 | −1.863 | 2.029 | 40.087 | 1.00 | 23.05 | Z | C |
| ATOM | 11598 | CE3 | TRP | Z | 36 | −2.557 | 1.352 | 42.303 | 1.00 | 26.55 | Z | C |
| ATOM | 11599 | CD1 | TRP | Z | 36 | 0.221 | 1.248 | 39.991 | 1.00 | 28.72 | Z | C |
| ATOM | 11600 | NE1 | TRP | Z | 36 | −0.754 | 1.929 | 39.296 | 1.00 | 22.76 | Z | N |
| ATOM | 11601 | CZ2 | TRP | Z | 36 | −3.098 | 2.617 | 39.828 | 1.00 | 25.24 | Z | C |
| ATOM | 11602 | CZ3 | TRP | Z | 36 | −3.788 | 1.938 | 42.041 | 1.00 | 23.53 | Z | C |
| ATOM | 11603 | CH2 | TRP | Z | 36 | −4.045 | 2.562 | 40.815 | 1.00 | 23.95 | Z | C |
| ATOM | 11604 | C | TRP | Z | 36 | 1.234 | 0.681 | 44.692 | 1.00 | 23.54 | Z | C |
| ATOM | 11605 | O | TRP | Z | 36 | 2.230 | −0.036 | 44.822 | 1.00 | 21.76 | Z | O |
| ATOM | 11606 | N | VAL | Z | 37 | 0.370 | 0.961 | 45.665 | 1.00 | 23.46 | Z | N |
| ATOM | 11607 | CA | VAL | Z | 37 | 0.514 | 0.469 | 47.037 | 1.00 | 21.80 | Z | C |
| ATOM | 11608 | CB | VAL | Z | 37 | 0.742 | 1.678 | 47.983 | 1.00 | 26.09 | Z | C |
| ATOM | 11609 | CG1 | VAL | Z | 37 | 0.648 | 1.277 | 49.455 | 1.00 | 14.09 | Z | C |
| ATOM | 11610 | CG2 | VAL | Z | 37 | 2.096 | 2.324 | 47.658 | 1.00 | 19.47 | Z | C |
| ATOM | 11611 | C | VAL | Z | 37 | −0.711 | −0.306 | 47.505 | 1.00 | 23.65 | Z | C |
| ATOM | 11612 | O | VAL | Z | 37 | −1.856 | 0.117 | 47.283 | 1.00 | 18.16 | Z | O |
| ATOM | 11613 | N | LYS | Z | 38 | −0.456 | −1.412 | 48.199 | 1.00 | 22.35 | Z | N |
| ATOM | 11614 | CA | LYS | Z | 38 | −1.512 | −2.272 | 48.719 | 1.00 | 24.65 | Z | C |
| ATOM | 11615 | CB | LYS | Z | 38 | −1.248 | −3.719 | 48.302 | 1.00 | 22.49 | Z | C |
| ATOM | 11616 | CG | LYS | Z | 38 | −2.303 | −4.696 | 48.792 | 1.00 | 24.89 | Z | C |
| ATOM | 11617 | CD | LYS | Z | 38 | −1.884 | −6.128 | 48.529 | 1.00 | 26.70 | Z | C |
| ATOM | 11618 | CE | LYS | Z | 38 | −2.832 | −7.109 | 49.211 | 1.00 | 31.22 | Z | C |
| ATOM | 11619 | NZ | LYS | Z | 38 | −2.478 | −8.520 | 48.934 | 1.00 | 26.00 | Z | N |
| ATOM | 11620 | C | LYS | Z | 38 | −1.655 | −2.198 | 50.241 | 1.00 | 27.14 | Z | C |
| ATOM | 11621 | O | LYS | Z | 38 | −0.712 | −2.492 | 50.973 | 1.00 | 26.79 | Z | O |
| ATOM | 11622 | N | GLN | Z | 39 | −2.848 | −1.834 | 50.708 | 1.00 | 31.22 | Z | N |
| ATOM | 11623 | CA | GLN | Z | 39 | −3.131 | −1.742 | 52.147 | 1.00 | 31.88 | Z | C |
| ATOM | 11624 | CB | GLN | Z | 39 | −3.714 | −0.369 | 52.499 | 1.00 | 29.00 | Z | C |
| ATOM | 11625 | CG | GLN | Z | 39 | −4.030 | −0.202 | 53.960 | 1.00 | 24.18 | Z | C |
| ATOM | 11626 | CD | GLN | Z | 39 | −4.417 | 1.216 | 54.324 | 1.00 | 31.05 | Z | C |
| ATOM | 11627 | OE1 | GLN | Z | 39 | −5.298 | 1.819 | 53.701 | 1.00 | 35.63 | Z | O |
| ATOM | 11628 | NE2 | GLN | Z | 39 | −3.766 | 1.759 | 55.354 | 1.00 | 32.63 | Z | N |
| ATOM | 11629 | C | GLN | Z | 39 | −4.092 | −2.842 | 52.596 | 1.00 | 30.55 | Z | C |
| ATOM | 11630 | O | GLN | Z | 39 | −5.278 | −2.804 | 52.273 | 1.00 | 31.59 | Z | O |
| ATOM | 11631 | N | SER | Z | 40 | −3.557 | −3.801 | 53.351 | 1.00 | 34.51 | Z | N |
| ATOM | 11632 | CA | SER | Z | 40 | −4.299 | −4.941 | 53.894 | 1.00 | 35.75 | Z | C |
| ATOM | 11633 | CB | SER | Z | 40 | −3.378 | −6.162 | 53.990 | 1.00 | 33.91 | Z | C |
| ATOM | 11634 | OG | SER | Z | 40 | −2.865 | −6.551 | 52.737 | 1.00 | 40.31 | Z | O |
| ATOM | 11635 | C | SER | Z | 40 | −4.854 | −4.684 | 55.307 | 1.00 | 40.21 | Z | C |
| ATOM | 11636 | O | SER | Z | 40 | −4.106 | −4.311 | 56.202 | 1.00 | 41.93 | Z | O |
| ATOM | 11637 | N | HIS | Z | 41 | −6.145 | −4.939 | 55.509 | 1.00 | 41.58 | Z | N |
| ATOM | 11638 | CA | HIS | Z | 41 | −6.808 | −4.789 | 56.811 | 1.00 | 47.30 | Z | C |
| ATOM | 11639 | CB | HIS | Z | 41 | −6.087 | −5.638 | 57.863 | 1.00 | 46.06 | Z | C |
| ATOM | 11640 | CG | HIS | Z | 41 | −5.854 | −7.050 | 57.432 | 1.00 | 50.29 | Z | C |
| ATOM | 11641 | CD2 | HIS | Z | 41 | −6.722 | −8.047 | 57.131 | 1.00 | 53.44 | Z | C |
| ATOM | 11642 | ND1 | HIS | Z | 41 | −4.592 | −7.565 | 57.225 | 1.00 | 54.67 | Z | N |
| ATOM | 11643 | CE1 | HIS | Z | 41 | −4.692 | −8.818 | 56.814 | 1.00 | 52.86 | Z | C |
| ATOM | 11644 | NE2 | HIS | Z | 41 | −5.973 | −9.136 | 56.748 | 1.00 | 53.26 | Z | N |
| ATOM | 11645 | C | HIS | Z | 41 | −7.051 | −3.381 | 57.362 | 1.00 | 48.52 | Z | C |
| ATOM | 11646 | O | HIS | Z | 41 | −7.728 | −3.229 | 58.376 | 1.00 | 57.96 | Z | O |
| ATOM | 11647 | N | GLY | Z | 42 | −6.549 | −2.354 | 56.685 | 1.00 | 54.33 | Z | N |
| ATOM | 11648 | CA | GLY | Z | 42 | −6.739 | −0.995 | 57.174 | 1.00 | 55.61 | Z | C |
| ATOM | 11649 | C | GLY | Z | 42 | −5.450 | −0.378 | 57.701 | 1.00 | 61.72 | Z | C |
| ATOM | 11650 | O | GLY | Z | 42 | −5.391 | 0.826 | 57.983 | 1.00 | 61.81 | Z | O |
| ATOM | 11651 | N | LYS | Z | 43 | −4.432 | −1.226 | 57.855 | 1.00 | 61.71 | Z | N |
| ATOM | 11652 | CA | LYS | Z | 43 | −3.095 | −0.853 | 58.322 | 1.00 | 65.49 | Z | C |
| ATOM | 11653 | CB | LYS | Z | 43 | −2.945 | −1.187 | 59.816 | 1.00 | 67.21 | Z | C |
| ATOM | 11654 | CG | LYS | Z | 43 | −3.161 | −2.664 | 60.168 | 1.00 | 69.12 | Z | C |
| ATOM | 11655 | CD | LYS | Z | 43 | −3.300 | −2.908 | 61.677 | 1.00 | 71.32 | Z | C |
| ATOM | 11656 | CE | LYS | Z | 43 | −2.044 | −2.524 | 62.471 | 1.00 | 72.36 | Z | C |
| ATOM | 11657 | NZ | LYS | Z | 43 | −1.861 | −1.047 | 62.612 | 1.00 | 73.25 | Z | N |
| ATOM | 11658 | C | LYS | Z | 43 | −2.140 | −1.700 | 57.476 | 1.00 | 63.80 | Z | C |
| ATOM | 11659 | O | LYS | Z | 43 | −2.596 | −2.446 | 56.617 | 1.00 | 69.81 | Z | O |
| ATOM | 11660 | N | ASN | Z | 44 | −0.833 | −1.589 | 57.690 | 1.00 | 62.29 | Z | N |
| ATOM | 11661 | CA | ASN | Z | 44 | 0.136 | −2.384 | 56.918 | 1.00 | 59.19 | Z | C |
| ATOM | 11662 | CB | ASN | Z | 44 | −0.030 | −3.877 | 57.234 | 1.00 | 61.83 | Z | C |
| ATOM | 11663 | CG | ASN | Z | 44 | 1.036 | −4.733 | 56.575 | 1.00 | 70.41 | Z | C |
| ATOM | 11664 | OD1 | ASN | Z | 44 | 0.765 | −5.428 | 55.588 | 1.00 | 72.41 | Z | O |
| ATOM | 11665 | ND2 | ASN | Z | 44 | 2.261 | −4.680 | 57.109 | 1.00 | 69.27 | Z | N |
| ATOM | 11666 | C | ASN | Z | 44 | 0.096 | −2.134 | 55.389 | 1.00 | 53.13 | Z | C |
| ATOM | 11667 | O | ASN | Z | 44 | −0.847 | −2.528 | 54.686 | 1.00 | 50.72 | Z | O |
| ATOM | 11668 | N | LEU | Z | 45 | 1.149 | −1.496 | 54.886 | 1.00 | 41.74 | Z | N |
| ATOM | 11669 | CA | LEU | Z | 45 | 1.263 | −1.155 | 53.475 | 1.00 | 29.78 | Z | C |
| ATOM | 11670 | CB | LEU | Z | 45 | 1.687 | 0.297 | 53.348 | 1.00 | 31.45 | Z | C |
| ATOM | 11671 | CG | LEU | Z | 45 | 0.809 | 1.301 | 54.068 | 1.00 | 29.09 | Z | C |
| ATOM | 11672 | CD1 | LEU | Z | 45 | 1.413 | 2.685 | 53.902 | 1.00 | 27.94 | Z | C |
| ATOM | 11673 | CD2 | LEU | Z | 45 | −0.580 | 1.235 | 53.472 | 1.00 | 27.19 | Z | C |
| ATOM | 11674 | C | LEU | Z | 45 | 2.291 | −2.009 | 52.753 | 1.00 | 24.51 | Z | C |
| ATOM | 11675 | O | LEU | Z | 45 | 3.243 | −2.480 | 53.362 | 1.00 | 27.89 | Z | O |
| ATOM | 11676 | N | GLU | Z | 46 | 2.082 | −2.215 | 51.455 | 1.00 | 22.44 | Z | N |
| ATOM | 11677 | CA | GLU | Z | 46 | 3.016 | −2.978 | 50.607 | 1.00 | 25.29 | Z | C |
| ATOM | 11678 | CB | GLU | Z | 46 | 2.564 | −4.414 | 50.373 | 1.00 | 27.16 | Z | C |
| ATOM | 11679 | CG | GLU | Z | 46 | 2.999 | −5.427 | 51.398 | 1.00 | 33.03 | Z | C |
| ATOM | 11680 | CD | GLU | Z | 46 | 2.388 | −6.789 | 51.113 | 1.00 | 38.88 | Z | C |
| ATOM | 11681 | OE1 | GLU | Z | 46 | 1.213 | −6.840 | 50.685 | 1.00 | 38.55 | Z | O |
| ATOM | 11682 | OE2 | GLU | Z | 46 | 3.086 | −7.807 | 51.289 | 1.00 | 41.63 | Z | O |
| ATOM | 11683 | C | GLU | Z | 46 | 3.113 | −2.309 | 49.255 | 1.00 | 21.49 | Z | C |
| ATOM | 11684 | O | GLU | Z | 46 | 2.106 | −1.892 | 48.677 | 1.00 | 27.13 | Z | O |
| ATOM | 11685 | N | TRP | Z | 47 | 4.337 | −2.176 | 48.770 | 1.00 | 21.00 | Z | N |
| ATOM | 11686 | CA | TRP | Z | 47 | 4.589 | −1.571 | 47.466 | 1.00 | 24.00 | Z | C |
| ATOM | 11687 | CB | TRP | Z | 47 | 5.996 | −0.951 | 47.434 | 1.00 | 24.35 | Z | C |
| ATOM | 11688 | CG | TRP | Z | 47 | 6.398 | −0.357 | 46.105 | 1.00 | 23.07 | Z | C |
| ATOM | 11689 | CD2 | TRP | Z | 47 | 7.299 | −0.931 | 45.152 | 1.00 | 19.26 | Z | C |
| ATOM | 11690 | CE2 | TRP | Z | 47 | 7.461 | 0.006 | 44.115 | 1.00 | 18.09 | Z | C |
| ATOM | 11691 | CE3 | TRP | Z | 47 | 7.994 | −2.147 | 45.079 | 1.00 | 25.71 | Z | C |
| ATOM | 11692 | CD1 | TRP | Z | 47 | 6.045 | 0.859 | 45.613 | 1.00 | 19.28 | Z | C |
| ATOM | 11693 | NE1 | TRP | Z | 47 | 6.684 | 1.090 | 44.420 | 1.00 | 18.72 | Z | N |
| ATOM | 11694 | CZ2 | TRP | Z | 47 | 8.295 | −0.228 | 43.014 | 1.00 | 22.49 | Z | C |
| ATOM | 11695 | CZ3 | TRP | Z | 47 | 8.826 | −2.381 | 43.982 | 1.00 | 23.80 | Z | C |
| ATOM | 11696 | CH2 | TRP | Z | 47 | 8.968 | −1.423 | 42.967 | 1.00 | 20.91 | Z | C |
| ATOM | 11697 | C | TRP | Z | 47 | 4.471 | −2.663 | 46.400 | 1.00 | 22.42 | Z | C |
| ATOM | 11698 | O | TRP | Z | 47 | 5.086 | −3.721 | 46.524 | 1.00 | 25.38 | Z | O |
| ATOM | 11699 | N | ILE | Z | 48 | 3.678 | −2.396 | 45.367 | 1.00 | 22.92 | Z | N |
| ATOM | 11700 | CA | ILE | Z | 48 | 3.461 | −3.350 | 44.283 | 1.00 | 26.63 | Z | C |
| ATOM | 11701 | CB | ILE | Z | 48 | 2.009 | −3.258 | 43.755 | 1.00 | 26.93 | Z | C |
| ATOM | 11702 | CG2 | ILE | Z | 48 | 1.845 | −4.113 | 42.513 | 1.00 | 19.77 | Z | C |
| ATOM | 11703 | CG1 | ILE | Z | 48 | 1.043 | −3.729 | 44.848 | 1.00 | 23.35 | Z | C |
| ATOM | 11704 | CD1 | ILE | Z | 48 | −0.349 | −3.182 | 44.700 | 1.00 | 25.26 | Z | C |
| ATOM | 11705 | C | ILE | Z | 48 | 4.456 | −3.144 | 43.147 | 1.00 | 24.67 | Z | C |
| ATOM | 11706 | O | ILE | Z | 48 | 5.104 | −4.085 | 42.689 | 1.00 | 24.76 | Z | O |
| ATOM | 11707 | N | GLY | Z | 49 | 4.590 | −1.899 | 42.718 | 1.00 | 22.91 | Z | N |
| ATOM | 11708 | CA | GLY | Z | 49 | 5.517 | −1.592 | 41.661 | 1.00 | 19.76 | Z | C |
| ATOM | 11709 | C | GLY | Z | 49 | 5.403 | −0.133 | 41.306 | 1.00 | 24.39 | Z | C |
| ATOM | 11710 | O | GLY | Z | 49 | 4.593 | 0.592 | 41.888 | 1.00 | 22.47 | Z | O |
| ATOM | 11711 | N | LEU | Z | 50 | 6.227 | 0.302 | 40.356 | 1.00 | 23.37 | Z | N |
| ATOM | 11712 | CA | LEU | Z | 50 | 6.198 | 1.684 | 39.914 | 1.00 | 21.94 | Z | C |
| ATOM | 11713 | CB | LEU | Z | 50 | 7.435 | 2.452 | 40.423 | 1.00 | 24.43 | Z | C |
| ATOM | 11714 | CG | LEU | Z | 50 | 8.840 | 2.077 | 39.915 | 1.00 | 27.67 | Z | C |
| ATOM | 11715 | CD1 | LEU | Z | 50 | 9.096 | 2.715 | 38.575 | 1.00 | 20.12 | Z | C |
| ATOM | 11716 | CD2 | LEU | Z | 50 | 9.891 | 2.536 | 40.908 | 1.00 | 29.48 | Z | C |
| ATOM | 11717 | C | LEU | Z | 50 | 6.159 | 1.715 | 38.402 | 1.00 | 23.82 | Z | C |
| ATOM | 11718 | O | LEU | Z | 50 | 6.518 | 0.746 | 37.735 | 1.00 | 20.49 | Z | O |
| ATOM | 11719 | N | VAL | Z | 51 | 5.722 | 2.843 | 37.869 | 1.00 | 19.87 | Z | N |
| ATOM | 11720 | CA | VAL | Z | 51 | 5.691 | 3.024 | 36.444 | 1.00 | 18.63 | Z | C |
| ATOM | 11721 | CB | VAL | Z | 51 | 4.278 | 2.809 | 35.854 | 1.00 | 22.69 | Z | C |
| ATOM | 11722 | CG1 | VAL | Z | 51 | 3.279 | 3.798 | 36.446 | 1.00 | 14.28 | Z | C |
| ATOM | 11723 | CG2 | VAL | Z | 51 | 4.324 | 2.958 | 34.334 | 1.00 | 20.22 | Z | C |
| ATOM | 11724 | C | VAL | Z | 51 | 6.143 | 4.437 | 36.117 | 1.00 | 22.02 | Z | C |
| ATOM | 11725 | O | VAL | Z | 51 | 5.761 | 5.397 | 36.787 | 1.00 | 21.36 | Z | O |
| ATOM | 11726 | N | HIS | Z | 52 | 6.981 | 4.545 | 35.094 | 1.00 | 23.52 | Z | N |
| ATOM | 11727 | CA | HIS | Z | 52 | 7.471 | 5.827 | 34.615 | 1.00 | 21.10 | Z | C |
| ATOM | 11728 | CB | HIS | Z | 52 | 8.916 | 5.679 | 34.114 | 1.00 | 14.17 | Z | C |
| ATOM | 11729 | CG | HIS | Z | 52 | 9.581 | 6.982 | 33.848 | 1.00 | 17.27 | Z | C |
| ATOM | 11730 | CD2 | HIS | Z | 52 | 10.865 | 7.383 | 34.014 | 1.00 | 15.83 | Z | C |
| ATOM | 11731 | ND1 | HIS | Z | 52 | 8.889 | 8.080 | 33.386 | 1.00 | 18.41 | Z | N |
| ATOM | 11732 | CE1 | HIS | Z | 52 | 9.719 | 9.103 | 33.277 | 1.00 | 21.95 | Z | C |
| ATOM | 11733 | NE2 | HIS | Z | 52 | 10.923 | 8.706 | 33.650 | 1.00 | 17.88 | Z | N |
| ATOM | 11734 | C | HIS | Z | 52 | 6.499 | 6.142 | 33.467 | 1.00 | 18.27 | Z | C |
| ATOM | 11735 | O | HIS | Z | 52 | 6.472 | 5.450 | 32.465 | 1.00 | 20.35 | Z | O |
| ATOM | 11736 | N | PRO | Z | 52A | 5.672 | 7.175 | 33.622 | 1.00 | 19.79 | Z | N |
| ATOM | 11737 | CD | PRO | Z | 52A | 5.508 | 8.026 | 34.806 | 1.00 | 22.91 | Z | C |
| ATOM | 11738 | CA | PRO | Z | 52A | 4.698 | 7.546 | 32.598 | 1.00 | 25.17 | Z | C |
| ATOM | 11739 | CB | PRO | Z | 52A | 3.881 | 8.644 | 33.276 | 1.00 | 27.33 | Z | C |
| ATOM | 11740 | CG | PRO | Z | 52A | 4.055 | 8.374 | 34.720 | 1.00 | 24.91 | Z | C |
| ATOM | 11741 | C | PRO | Z | 52A | 5.216 | 8.019 | 31.265 | 1.00 | 29.63 | Z | C |
| ATOM | 11742 | O | PRO | Z | 52A | 4.459 | 8.024 | 30.298 | 1.00 | 38.19 | Z | O |
| ATOM | 11743 | N | HIS | Z | 53 | 6.470 | 8.452 | 31.189 | 1.00 | 26.93 | Z | N |
| ATOM | 11744 | CA | HIS | Z | 53 | 6.983 | 8.921 | 29.898 | 1.00 | 26.85 | Z | C |
| ATOM | 11745 | CB | HIS | Z | 53 | 8.342 | 9.636 | 30.036 | 1.00 | 21.20 | Z | C |
| ATOM | 11746 | CG | HIS | Z | 53 | 8.664 | 10.538 | 28.885 | 1.00 | 16.38 | Z | C |
| ATOM | 11747 | CD2 | HIS | Z | 53 | 8.135 | 11.730 | 28.510 | 1.00 | 17.03 | Z | C |
| ATOM | 11748 | ND1 | HIS | Z | 53 | 9.601 | 10.220 | 27.926 | 1.00 | 18.47 | Z | N |
| ATOM | 11749 | CE1 | HIS | Z | 53 | 9.636 | 11.172 | 27.009 | 1.00 | 12.33 | Z | C |
| ATOM | 11750 | NE2 | HIS | Z | 53 | 8.756 | 12.100 | 27.338 | 1.00 | 18.87 | Z | N |
| ATOM | 11751 | C | HIS | Z | 53 | 7.091 | 7.801 | 28.865 | 1.00 | 27.82 | Z | C |
| ATOM | 11752 | O | HIS | Z | 53 | 6.947 | 8.052 | 27.672 | 1.00 | 30.78 | Z | O |
| ATOM | 11753 | N | ASN | Z | 54 | 7.313 | 6.568 | 29.316 | 1.00 | 29.89 | Z | N |
| ATOM | 11754 | CA | ASN | Z | 54 | 7.447 | 5.445 | 28.380 | 1.00 | 38.28 | Z | C |
| ATOM | 11755 | CB | ASN | Z | 54 | 8.905 | 5.297 | 27.923 | 1.00 | 41.10 | Z | C |
| ATOM | 11756 | CG | ASN | Z | 54 | 9.866 | 5.127 | 29.073 | 1.00 | 41.39 | Z | C |
| ATOM | 11757 | OD1 | ASN | Z | 54 | 9.517 | 4.636 | 30.146 | 1.00 | 49.68 | Z | O |
| ATOM | 11758 | ND2 | ASN | Z | 54 | 11.090 | 5.550 | 28.856 | 1.00 | 51.08 | Z | N |
| ATOM | 11759 | C | ASN | Z | 54 | 6.948 | 4.087 | 28.869 | 1.00 | 36.44 | Z | C |
| ATOM | 11760 | O | ASN | Z | 54 | 7.225 | 3.058 | 28.239 | 1.00 | 30.34 | Z | O |
| ATOM | 11761 | N | GLY | Z | 55 | 6.279 | 4.084 | 30.018 | 1.00 | 30.96 | Z | N |
| ATOM | 11762 | CA | GLY | Z | 55 | 5.754 | 2.853 | 30.567 | 1.00 | 22.99 | Z | C |
| ATOM | 11763 | C | GLY | Z | 55 | 6.782 | 1.900 | 31.135 | 1.00 | 25.03 | Z | C |
| ATOM | 11764 | O | GLY | Z | 55 | 6.507 | 0.707 | 31.238 | 1.00 | 29.46 | Z | O |
| ATOM | 11765 | N | ALA | Z | 56 | 7.970 | 2.391 | 31.478 | 1.00 | 21.80 | Z | N |
| ATOM | 11766 | CA | ALA | Z | 56 | 8.987 | 1.511 | 32.062 | 1.00 | 21.47 | Z | C |
| ATOM | 11767 | CB | ALA | Z | 56 | 10.325 | 2.222 | 32.146 | 1.00 | 17.41 | Z | C |
| ATOM | 11768 | C | ALA | Z | 56 | 8.488 | 1.126 | 33.456 | 1.00 | 22.30 | Z | C |
| ATOM | 11769 | O | ALA | Z | 56 | 7.958 | 1.972 | 34.174 | 1.00 | 23.08 | Z | O |
| ATOM | 11770 | N | ILE | Z | 57 | 8.587 | −0.149 | 33.824 | 1.00 | 24.38 | Z | N |
| ATOM | 11771 | CA | ILE | Z | 57 | 8.097 | −0.555 | 35.135 | 1.00 | 25.02 | Z | C |
| ATOM | 11772 | CB | ILE | Z | 57 | 6.693 | −1.314 | 35.095 | 1.00 | 27.42 | Z | C |
| ATOM | 11773 | CG2 | ILE | Z | 57 | 5.678 | −0.574 | 34.234 | 1.00 | 28.31 | Z | C |
| ATOM | 11774 | CG1 | ILE | Z | 57 | 6.842 | −2.745 | 34.573 | 1.00 | 39.24 | Z | C |
| ATOM | 11775 | CD1 | ILE | Z | 57 | 6.842 | −2.894 | 33.047 | 1.00 | 43.11 | Z | C |
| ATOM | 11776 | C | ILE | Z | 57 | 9.064 | −1.402 | 35.936 | 1.00 | 26.87 | Z | C |
| ATOM | 11777 | O | ILE | Z | 57 | 9.955 | −2.053 | 35.393 | 1.00 | 27.11 | Z | O |
| ATOM | 11778 | N | THR | Z | 58 | 8.884 | −1.346 | 37.250 | 1.00 | 27.10 | Z | N |
| ATOM | 11779 | CA | THR | Z | 58 | 9.675 | −2.114 | 38.181 | 1.00 | 28.56 | Z | C |
| ATOM | 11780 | CB | THR | Z | 58 | 10.724 | −1.262 | 38.900 | 1.00 | 33.72 | Z | C |
| ATOM | 11781 | OG1 | THR | Z | 58 | 11.548 | −0.597 | 37.929 | 1.00 | 36.14 | Z | O |
| ATOM | 11782 | CG2 | THR | Z | 58 | 11.612 | −2.152 | 39.784 | 1.00 | 33.32 | Z | C |
| ATOM | 11783 | C | THR | Z | 58 | 8.644 | −2.657 | 39.157 | 1.00 | 31.85 | Z | C |
| ATOM | 11784 | O | THR | Z | 58 | 7.861 | −1.901 | 39.743 | 1.00 | 29.06 | Z | O |
| ATOM | 11785 | N | TYR | Z | 59 | 8.580 | −3.984 | 39.211 | 1.00 | 31.86 | Z | N |
| ATOM | 11786 | CA | TYR | Z | 59 | 7.654 | −4.724 | 40.051 | 1.00 | 28.55 | Z | C |
| ATOM | 11787 | CB | TYR | Z | 59 | 7.126 | −5.945 | 39.286 | 1.00 | 26.23 | Z | C |
| ATOM | 11788 | CG | TYR | Z | 59 | 6.144 | −5.655 | 38.192 | 1.00 | 27.09 | Z | C |
| ATOM | 11789 | CD1 | TYR | Z | 59 | 4.863 | −5.187 | 38.493 | 1.00 | 23.13 | Z | C |
| ATOM | 11790 | CE1 | TYR | Z | 59 | 3.964 | −4.885 | 37.500 | 1.00 | 21.87 | Z | C |
| ATOM | 11791 | CD2 | TYR | Z | 59 | 6.489 | −5.826 | 36.854 | 1.00 | 22.88 | Z | C |
| ATOM | 11792 | CE2 | TYR | Z | 59 | 5.577 | −5.527 | 35.844 | 1.00 | 24.69 | Z | C |
| ATOM | 11793 | CZ | TYR | Z | 59 | 4.322 | −5.052 | 36.182 | 1.00 | 19.46 | Z | C |
| ATOM | 11794 | OH | TYR | Z | 59 | 3.430 | −4.710 | 35.208 | 1.00 | 24.60 | Z | O |
| ATOM | 11795 | C | TYR | Z | 59 | 8.325 | −5.289 | 41.281 | 1.00 | 31.27 | Z | C |
| ATOM | 11796 | O | TYR | Z | 59 | 9.491 | −5.686 | 41.230 | 1.00 | 36.45 | Z | O |
| ATOM | 11797 | N | ASN | Z | 60 | 7.556 | −5.381 | 42.363 | 1.00 | 31.56 | Z | N |
| ATOM | 11798 | CA | ASN | Z | 60 | 8.012 | −6.011 | 43.593 | 1.00 | 32.90 | Z | C |
| ATOM | 11799 | CB | ASN | Z | 60 | 7.033 | −5.701 | 44.737 | 1.00 | 30.04 | Z | C |
| ATOM | 11800 | CG | ASN | Z | 60 | 7.427 | −6.367 | 46.051 | 1.00 | 32.12 | Z | C |
| ATOM | 11801 | OD1 | ASN | Z | 60 | 8.098 | −7.395 | 46.071 | 1.00 | 32.26 | Z | O |
| ATOM | 11802 | ND2 | ASN | Z | 60 | 7.015 | −5.770 | 47.159 | 1.00 | 36.80 | Z | N |
| ATOM | 11803 | C | ASN | Z | 60 | 7.880 | −7.485 | 43.181 | 1.00 | 36.34 | Z | C |
| ATOM | 11804 | O | ASN | Z | 60 | 6.852 | −7.872 | 42.621 | 1.00 | 36.73 | Z | O |
| ATOM | 11805 | N | GLN | Z | 61 | 8.906 | −8.295 | 43.426 | 1.00 | 42.66 | Z | N |
| ATOM | 11806 | CA | GLN | Z | 61 | 8.864 | −9.707 | 43.031 | 1.00 | 48.77 | Z | C |
| ATOM | 11807 | CB | GLN | Z | 61 | 10.178 | −10.438 | 43.384 | 1.00 | 50.21 | Z | C |
| ATOM | 11808 | CG | GLN | Z | 61 | 10.301 | −10.885 | 44.845 | 1.00 | 61.00 | Z | C |
| ATOM | 11809 | CD | GLN | Z | 61 | 11.487 | −11.824 | 45.117 | 1.00 | 64.13 | Z | C |
| ATOM | 11810 | OE1 | GLN | Z | 61 | 11.929 | −12.582 | 44.238 | 1.00 | 66.55 | Z | O |
| ATOM | 11811 | NE2 | GLN | Z | 61 | 11.989 | −11.791 | 46.354 | 1.00 | 63.88 | Z | N |
| ATOM | 11812 | C | GLN | Z | 61 | 7.663 | −10.484 | 43.584 | 1.00 | 49.12 | Z | C |
| ATOM | 11813 | O | GLN | Z | 61 | 7.179 | −11.418 | 42.939 | 1.00 | 51.82 | Z | O |
| ATOM | 11814 | N | LYS | Z | 62 | 7.176 | −10.098 | 44.762 | 1.00 | 43.83 | Z | N |
| ATOM | 11815 | CA | LYS | Z | 62 | 6.038 | −10.785 | 45.367 | 1.00 | 44.52 | Z | C |
| ATOM | 11816 | CB | LYS | Z | 62 | 5.834 | −10.297 | 46.813 | 1.00 | 51.43 | Z | C |
| ATOM | 11817 | CG | LYS | Z | 62 | 5.054 | −11.261 | 47.726 | 1.00 | 59.66 | Z | C |
| ATOM | 11818 | CD | LYS | Z | 62 | 3.568 | −10.892 | 47.878 | 1.00 | 62.31 | Z | C |
| ATOM | 11819 | CE | LYS | Z | 62 | 2.772 | −12.046 | 48.480 | 1.00 | 65.51 | Z | C |
| ATOM | 11820 | NZ | LYS | Z | 62 | 2.847 | −13.268 | 47.607 | 1.00 | 64.80 | Z | N |
| ATOM | 11821 | C | LYS | Z | 62 | 4.757 | −10.608 | 44.532 | 1.00 | 41.96 | Z | C |
| ATOM | 11822 | O | LYS | Z | 62 | 3.824 | −11.402 | 44.648 | 1.00 | 44.82 | Z | O |
| ATOM | 11823 | N | PHE | Z | 63 | 4.735 | −9.594 | 43.665 | 1.00 | 37.06 | Z | N |
| ATOM | 11824 | CA | PHE | Z | 63 | 3.576 | −9.321 | 42.810 | 1.00 | 34.47 | Z | C |
| ATOM | 11825 | CB | PHE | Z | 63 | 3.062 | −7.895 | 43.036 | 1.00 | 33.86 | Z | C |
| ATOM | 11826 | CG | PHE | Z | 63 | 2.491 | −7.667 | 44.391 | 1.00 | 35.04 | Z | C |
| ATOM | 11827 | CD1 | PHE | Z | 63 | 1.152 | −7.931 | 44.640 | 1.00 | 33.41 | Z | C |
| ATOM | 11828 | CD2 | PHE | Z | 63 | 3.297 | −7.220 | 45.434 | 1.00 | 33.67 | Z | C |
| ATOM | 11829 | CE1 | PHE | Z | 63 | 0.618 | −7.762 | 45.911 | 1.00 | 31.83 | Z | C |
| ATOM | 11830 | CE2 | PHE | Z | 63 | 2.777 | −7.048 | 46.707 | 1.00 | 33.66 | Z | C |
| ATOM | 11831 | CZ | PHE | Z | 63 | 1.434 | −7.320 | 46.948 | 1.00 | 36.96 | Z | C |
| ATOM | 11832 | C | PHE | Z | 63 | 3.856 | −9.493 | 41.317 | 1.00 | 36.70 | Z | C |
| ATOM | 11833 | O | PHE | Z | 63 | 3.016 | −9.128 | 40.486 | 1.00 | 32.70 | Z | O |
| ATOM | 11834 | N | LYS | Z | 64 | 5.028 | −10.030 | 40.980 | 1.00 | 38.02 | Z | N |
| ATOM | 11835 | CA | LYS | Z | 64 | 5.445 | −10.249 | 39.582 | 1.00 | 44.98 | Z | C |
| ATOM | 11836 | CB | LYS | Z | 64 | 6.691 | −11.171 | 39.528 | 1.00 | 47.52 | Z | C |
| ATOM | 11837 | CG | LYS | Z | 64 | 8.051 | −10.459 | 39.712 | 1.00 | 51.64 | Z | C |
| ATOM | 11838 | CD | LYS | Z | 64 | 9.222 | −11.457 | 39.924 | 1.00 | 54.54 | Z | C |
| ATOM | 11839 | CE | LYS | Z | 64 | 10.629 | −10.834 | 39.678 | 1.00 | 49.50 | Z | C |
| ATOM | 11840 | NZ | LYS | Z | 64 | 11.030 | −9.726 | 40.600 | 1.00 | 43.85 | Z | N |
| ATOM | 11841 | C | LYS | Z | 64 | 4.358 | −10.825 | 38.664 | 1.00 | 42.20 | Z | C |
| ATOM | 11842 | O | LYS | Z | 64 | 4.158 | −10.347 | 37.546 | 1.00 | 42.16 | Z | O |
| ATOM | 11843 | N | ASP | Z | 65 | 3.638 | −11.830 | 39.150 | 1.00 | 39.25 | Z | N |
| ATOM | 11844 | CA | ASP | Z | 65 | 2.605 | −12.456 | 38.335 | 1.00 | 47.29 | Z | C |
| ATOM | 11845 | CB | ASP | Z | 65 | 2.740 | −13.984 | 38.383 | 1.00 | 49.85 | Z | C |
| ATOM | 11846 | CG | ASP | Z | 65 | 4.156 | −14.455 | 38.075 | 1.00 | 57.12 | Z | C |
| ATOM | 11847 | OD1 | ASP | Z | 65 | 4.651 | −14.172 | 36.955 | 1.00 | 54.28 | Z | O |
| ATOM | 11848 | OD2 | ASP | Z | 65 | 4.777 | −15.088 | 38.964 | 1.00 | 52.79 | Z | O |
| ATOM | 11849 | C | ASP | Z | 65 | 1.168 | −12.056 | 38.651 | 1.00 | 42.65 | Z | C |
| ATOM | 11850 | O | ASP | Z | 65 | 0.246 | −12.491 | 37.964 | 1.00 | 45.68 | Z | O |
| ATOM | 11851 | N | LYS | Z | 66 | 0.988 | −11.208 | 39.661 | 1.00 | 42.04 | Z | N |
| ATOM | 11852 | CA | LYS | Z | 66 | −0.339 | −10.748 | 40.094 | 1.00 | 35.76 | Z | C |
| ATOM | 11853 | CB | LYS | Z | 66 | −0.355 | −10.550 | 41.616 | 1.00 | 34.55 | Z | C |
| ATOM | 11854 | CG | LYS | Z | 66 | −1.672 | −10.023 | 42.190 | 1.00 | 36.26 | Z | C |
| ATOM | 11855 | CD | LYS | Z | 66 | −2.843 | −10.983 | 41.967 | 1.00 | 37.59 | Z | C |
| ATOM | 11856 | CE | LYS | Z | 66 | −2.608 | −12.350 | 42.613 | 1.00 | 34.82 | Z | C |
| ATOM | 11857 | NZ | LYS | Z | 66 | −3.779 | −13.272 | 42.429 | 1.00 | 37.83 | Z | N |
| ATOM | 11858 | C | LYS | Z | 66 | −0.767 | −9.454 | 39.434 | 1.00 | 33.41 | Z | C |
| ATOM | 11859 | O | LYS | Z | 66 | −1.859 | −9.361 | 38.867 | 1.00 | 36.32 | Z | O |
| ATOM | 11860 | N | ALA | Z | 67 | 0.094 | −8.448 | 39.526 | 1.00 | 31.32 | Z | N |
| ATOM | 11861 | CA | ALA | Z | 67 | −0.205 | −7.144 | 38.958 | 1.00 | 27.89 | Z | C |
| ATOM | 11862 | CB | ALA | Z | 67 | 0.208 | −6.052 | 39.949 | 1.00 | 27.29 | Z | C |
| ATOM | 11863 | C | ALA | Z | 67 | 0.435 | −6.878 | 37.596 | 1.00 | 26.31 | Z | C |
| ATOM | 11864 | O | ALA | Z | 67 | 1.445 | −7.471 | 37.234 | 1.00 | 31.94 | Z | O |
| ATOM | 11865 | N | THR | Z | 68 | −0.167 | −5.961 | 36.857 | 1.00 | 22.23 | Z | N |
| ATOM | 11866 | CA | THR | Z | 68 | 0.335 | −5.563 | 35.566 | 1.00 | 24.29 | Z | C |
| ATOM | 11867 | CB | THR | Z | 68 | −0.447 | −6.223 | 34.401 | 1.00 | 21.28 | Z | C |
| ATOM | 11868 | OG1 | THR | Z | 68 | −0.107 | −7.606 | 34.343 | 1.00 | 23.65 | Z | O |
| ATOM | 11869 | CG2 | THR | Z | 68 | −0.097 | −5.581 | 33.063 | 1.00 | 15.94 | Z | C |
| ATOM | 11870 | C | THR | Z | 68 | 0.145 | −4.063 | 35.534 | 1.00 | 27.87 | Z | C |
| ATOM | 11871 | O | THR | Z | 68 | −0.981 | −3.585 | 35.522 | 1.00 | 33.89 | Z | O |
| ATOM | 11872 | N | LEU | Z | 69 | 1.253 | −3.329 | 35.583 | 1.00 | 28.75 | Z | N |
| ATOM | 11873 | CA | LEU | Z | 69 | 1.209 | −1.880 | 35.564 | 1.00 | 29.93 | Z | C |
| ATOM | 11874 | CB | LEU | Z | 69 | 2.387 | −1.292 | 36.337 | 1.00 | 31.23 | Z | C |
| ATOM | 11875 | CG | LEU | Z | 69 | 2.038 | −1.024 | 37.804 | 1.00 | 32.14 | Z | C |
| ATOM | 11876 | CD1 | LEU | Z | 69 | 1.295 | −2.188 | 38.412 | 1.00 | 29.85 | Z | C |
| ATOM | 11877 | CD2 | LEU | Z | 69 | 3.288 | −0.737 | 38.587 | 1.00 | 32.06 | Z | C |
| ATOM | 11878 | C | LEU | Z | 69 | 1.160 | −1.361 | 34.146 | 1.00 | 29.90 | Z | C |
| ATOM | 11879 | O | LEU | Z | 69 | 1.888 | −1.824 | 33.268 | 1.00 | 35.51 | Z | O |
| ATOM | 11880 | N | THR | Z | 70 | 0.328 | −0.356 | 33.939 | 1.00 | 23.93 | Z | N |
| ATOM | 11881 | CA | THR | Z | 70 | 0.156 | 0.189 | 32.622 | 1.00 | 21.13 | Z | C |
| ATOM | 11882 | CB | THR | Z | 70 | −0.950 | −0.640 | 31.902 | 1.00 | 26.82 | Z | C |
| ATOM | 11883 | OG1 | THR | Z | 70 | −0.405 | −1.262 | 30.733 | 1.00 | 32.40 | Z | O |
| ATOM | 11884 | CG2 | THR | Z | 70 | −2.168 | 0.193 | 31.557 | 1.00 | 29.23 | Z | C |
| ATOM | 11885 | C | THR | Z | 70 | −0.185 | 1.664 | 32.708 | 1.00 | 19.26 | Z | C |
| ATOM | 11886 | O | THR | Z | 70 | −0.715 | 2.123 | 33.710 | 1.00 | 25.84 | Z | O |
| ATOM | 11887 | N | VAL | Z | 71 | 0.088 | 2.395 | 31.633 | 1.00 | 23.12 | Z | N |
| ATOM | 11888 | CA | VAL | Z | 71 | −0.176 | 3.824 | 31.591 | 1.00 | 26.46 | Z | C |
| ATOM | 11889 | CB | VAL | Z | 71 | 1.148 | 4.635 | 31.775 | 1.00 | 29.86 | Z | C |
| ATOM | 11890 | CG1 | VAL | Z | 71 | 1.914 | 4.736 | 30.480 | 1.00 | 22.96 | Z | C |
| ATOM | 11891 | CG2 | VAL | Z | 71 | 0.864 | 6.001 | 32.264 | 1.00 | 29.31 | Z | C |
| ATOM | 11892 | C | VAL | Z | 71 | −0.780 | 4.189 | 30.244 | 1.00 | 28.55 | Z | C |
| ATOM | 11893 | O | VAL | Z | 71 | −0.572 | 3.479 | 29.266 | 1.00 | 33.65 | Z | O |
| ATOM | 11894 | N | ASP | Z | 72 | −1.489 | 5.315 | 30.199 | 1.00 | 29.02 | Z | N |
| ATOM | 11895 | CA | ASP | Z | 72 | −2.117 | 5.831 | 28.982 | 1.00 | 30.84 | Z | C |
| ATOM | 11896 | CB | ASP | Z | 72 | −3.616 | 5.478 | 28.978 | 1.00 | 44.00 | Z | C |
| ATOM | 11897 | CG | ASP | Z | 72 | −4.396 | 6.097 | 27.799 | 1.00 | 50.13 | Z | C |
| ATOM | 11898 | OD1 | ASP | Z | 72 | −3.797 | 6.729 | 26.895 | 1.00 | 49.19 | Z | O |
| ATOM | 11899 | OD2 | ASP | Z | 72 | −5.641 | 5.934 | 27.786 | 1.00 | 56.99 | Z | O |
| ATOM | 11900 | C | ASP | Z | 72 | −1.915 | 7.349 | 29.018 | 1.00 | 30.38 | Z | C |
| ATOM | 11901 | O | ASP | Z | 72 | −2.552 | 8.049 | 29.807 | 1.00 | 28.15 | Z | O |
| ATOM | 11902 | N | ARG | Z | 73 | −1.059 | 7.847 | 28.129 | 1.00 | 24.98 | Z | N |
| ATOM | 11903 | CA | ARG | Z | 73 | −0.720 | 9.263 | 28.071 | 1.00 | 27.15 | Z | C |
| ATOM | 11904 | CB | ARG | Z | 73 | 0.635 | 9.447 | 27.377 | 1.00 | 29.16 | Z | C |
| ATOM | 11905 | CG | ARG | Z | 73 | 1.807 | 8.837 | 28.168 | 1.00 | 32.16 | Z | C |
| ATOM | 11906 | CD | ARG | Z | 73 | 3.150 | 8.932 | 27.452 | 1.00 | 29.27 | Z | C |
| ATOM | 11907 | NE | ARG | Z | 73 | 3.362 | 7.819 | 26.536 | 1.00 | 30.51 | Z | N |
| ATOM | 11908 | CZ | ARG | Z | 73 | 3.783 | 6.610 | 26.904 | 1.00 | 34.91 | Z | C |
| ATOM | 11909 | NH1 | ARG | Z | 73 | 4.045 | 6.359 | 28.182 | 1.00 | 33.85 | Z | N |
| ATOM | 11910 | NH2 | ARG | Z | 73 | 3.920 | 5.641 | 26.001 | 1.00 | 30.44 | Z | N |
| ATOM | 11911 | C | ARG | Z | 73 | −1.745 | 10.215 | 27.479 | 1.00 | 31.85 | Z | C |
| ATOM | 11912 | O | ARG | Z | 73 | −1.753 | 11.402 | 27.810 | 1.00 | 33.09 | Z | O |
| ATOM | 11913 | N | SER | Z | 74 | −2.607 | 9.716 | 26.600 | 1.00 | 40.65 | Z | N |
| ATOM | 11914 | CA | SER | Z | 74 | −3.616 | 10.575 | 25.979 | 1.00 | 42.59 | Z | C |
| ATOM | 11915 | CB | SER | Z | 74 | −4.315 | 9.859 | 24.812 | 1.00 | 45.99 | Z | C |
| ATOM | 11916 | OG | SER | Z | 74 | −4.717 | 8.543 | 25.153 | 1.00 | 50.75 | Z | O |
| ATOM | 11917 | C | SER | Z | 74 | −4.630 | 11.078 | 26.994 | 1.00 | 41.47 | Z | C |
| ATOM | 11918 | O | SER | Z | 74 | −5.123 | 12.202 | 26.874 | 1.00 | 42.10 | Z | O |
| ATOM | 11919 | N | SER | Z | 75 | −4.883 | 10.269 | 28.024 | 1.00 | 39.91 | Z | N |
| ATOM | 11920 | CA | SER | Z | 75 | −5.832 | 10.619 | 29.079 | 1.00 | 37.57 | Z | C |
| ATOM | 11921 | CB | SER | Z | 75 | −6.955 | 9.588 | 29.135 | 1.00 | 36.97 | Z | C |
| ATOM | 11922 | OG | SER | Z | 75 | −6.437 | 8.301 | 29.424 | 1.00 | 39.52 | Z | O |
| ATOM | 11923 | C | SER | Z | 75 | −5.169 | 10.755 | 30.456 | 1.00 | 37.42 | Z | C |
| ATOM | 11924 | O | SER | Z | 75 | −5.856 | 10.840 | 31.474 | 1.00 | 36.67 | Z | O |
| ATOM | 11925 | N | THR | Z | 76 | −3.835 | 10.761 | 30.470 | 1.00 | 33.94 | Z | N |
| ATOM | 11926 | CA | THR | Z | 76 | −3.028 | 10.919 | 31.684 | 1.00 | 21.76 | Z | C |
| ATOM | 11927 | CB | THR | Z | 76 | −2.932 | 12.417 | 32.112 | 1.00 | 23.35 | Z | C |
| ATOM | 11928 | OG1 | THR | Z | 76 | −4.205 | 12.883 | 32.587 | 1.00 | 24.64 | Z | O |
| ATOM | 11929 | CG2 | THR | Z | 76 | −2.529 | 13.288 | 30.938 | 1.00 | 15.18 | Z | C |
| ATOM | 11930 | C | THR | Z | 76 | −3.521 | 10.080 | 32.853 | 1.00 | 24.79 | Z | C |
| ATOM | 11931 | O | THR | Z | 76 | −3.808 | 10.610 | 33.935 | 1.00 | 16.89 | Z | O |
| ATOM | 11932 | N | THR | Z | 77 | −3.687 | 8.780 | 32.623 | 1.00 | 21.08 | Z | N |
| ATOM | 11933 | CA | THR | Z | 77 | −4.130 | 7.907 | 33.699 | 1.00 | 26.96 | Z | C |
| ATOM | 11934 | CB | THR | Z | 77 | −5.685 | 7.553 | 33.663 | 1.00 | 27.83 | Z | C |
| ATOM | 11935 | OG1 | THA | Z | 77 | −5.894 | 6.211 | 33.219 | 1.00 | 34.25 | Z | O |
| ATOM | 11936 | CG2 | THR | Z | 77 | −6.460 | 8.496 | 32.800 | 1.00 | 25.23 | Z | C |
| ATOM | 11937 | C | THR | Z | 77 | −3.276 | 6.658 | 33.790 | 1.00 | 25.76 | Z | C |
| ATOM | 11938 | O | THR | Z | 77 | −2.702 | 6.208 | 32.793 | 1.00 | 31.16 | Z | O |
| ATOM | 11939 | N | ALA | Z | 78 | −3.128 | 6.141 | 35.005 | 1.00 | 21.73 | Z | N |
| ATOM | 11940 | CA | ALA | Z | 78 | −2.334 | 4.935 | 35.204 | 1.00 | 20.06 | Z | C |
| ATOM | 11941 | CB | ALA | Z | 78 | −1.305 | 5.142 | 36.302 | 1.00 | 17.11 | Z | C |
| ATOM | 11942 | C | ALA | Z | 78 | −3.306 | 3.838 | 35.579 | 1.00 | 21.18 | Z | C |
| ATOM | 11943 | O | ALA | Z | 78 | −4.416 | 4.121 | 36.047 | 1.00 | 24.65 | Z | O |
| ATOM | 11944 | N | TYR | Z | 79 | −2.895 | 2.594 | 35.379 | 1.00 | 15.45 | Z | N |
| ATOM | 11945 | CA | TYR | Z | 79 | −3.758 | 1.468 | 35.670 | 1.00 | 22.66 | Z | C |
| ATOM | 11946 | CB | TYR | Z | 79 | −4.404 | 0.899 | 34.382 | 1.00 | 24.89 | Z | C |
| ATOM | 11947 | CG | TYR | Z | 79 | −5.283 | 1.820 | 33.545 | 1.00 | 28.24 | Z | C |
| ATOM | 11948 | CD1 | TYR | Z | 79 | −4.743 | 2.616 | 32.535 | 1.00 | 27.76 | Z | C |
| ATOM | 11949 | CE1 | TYR | Z | 79 | −5.568 | 3.424 | 31.725 | 1.00 | 30.12 | Z | C |
| ATOM | 11950 | CD2 | TYR | Z | 79 | −6.665 | 1.848 | 33.727 | 1.00 | 31.49 | Z | C |
| ATOM | 11951 | CE2 | TYR | Z | 79 | −7.494 | 2.645 | 32.925 | 1.00 | 32.66 | Z | C |
| ATOM | 11952 | CZ | TYR | Z | 79 | −6.941 | 3.431 | 31.931 | 1.00 | 32.16 | Z | C |
| ATOM | 11953 | OH | TYR | Z | 79 | −7.760 | 4.247 | 31.178 | 1.00 | 34.41 | Z | O |
| ATOM | 11954 | C | TYR | Z | 79 | −2.968 | 0.337 | 36.289 | 1.00 | 23.66 | Z | C |
| ATOM | 11955 | O | TYR | Z | 79 | −1.770 | 0.199 | 36.046 | 1.00 | 25.42 | Z | O |
| ATOM | 11956 | N | ILE | Z | 80 | −3.682 | −0.487 | 37.055 | 1.00 | 27.81 | Z | N |
| ATOM | 11957 | CA | ILE | Z | 80 | −3.147 | −1.689 | 37.689 | 1.00 | 27.29 | Z | C |
| ATOM | 11958 | CB | ILE | Z | 80 | −2.864 | −1.519 | 39.216 | 1.00 | 23.15 | Z | C |
| ATOM | 11959 | CG2 | ILE | Z | 80 | −4.093 | −1.023 | 39.954 | 1.00 | 18.26 | Z | C |
| ATOM | 11960 | CG1 | ILE | Z | 80 | −2.371 | −2.844 | 39.799 | 1.00 | 18.60 | Z | C |
| ATOM | 11961 | CD1 | ILE | Z | 80 | −1.819 | −2.719 | 41.214 | 1.00 | 21.46 | Z | C |
| ATOM | 11962 | C | ILE | Z | 80 | −4.202 | −2.780 | 37.472 | 1.00 | 29.74 | Z | C |
| ATOM | 11963 | O | ILE | Z | 80 | −5.385 | −2.597 | 37.760 | 1.00 | 31.08 | Z | O |
| ATOM | 11964 | N | GLU | Z | 81 | −3.775 | −3.875 | 36.869 | 1.00 | 31.03 | Z | N |
| ATOM | 11965 | CA | GLU | Z | 81 | −4.653 | −4.983 | 36.608 | 1.00 | 29.69 | Z | C |
| ATOM | 11966 | CB | GLU | Z | 81 | −4.579 | −5.369 | 35.135 | 1.00 | 37.59 | Z | C |
| ATOM | 11967 | CG | GLU | Z | 81 | −5.395 | −6.596 | 34.739 | 1.00 | 38.99 | Z | C |
| ATOM | 11968 | CD | GLU | Z | 81 | −5.435 | −6.778 | 33.241 | 1.00 | 47.45 | Z | C |
| ATOM | 11969 | OE1 | GLU | Z | 81 | −4.364 | −7.030 | 32.640 | 1.00 | 53.67 | Z | O |
| ATOM | 11970 | OE2 | GLU | Z | 81 | −6.533 | −6.643 | 32.657 | 1.00 | 50.93 | Z | O |
| ATOM | 11971 | C | GLU | Z | 81 | −4.219 | −6.149 | 37.456 | 1.00 | 29.82 | Z | C |
| ATOM | 11972 | O | GLU | Z | 81 | −3.107 | −6.634 | 37.296 | 1.00 | 34.58 | Z | O |
| ATOM | 11973 | N | LEU | Z | 82 | −5.074 | −6.546 | 38.400 | 1.00 | 31.08 | Z | N |
| ATOM | 11974 | CA | LEU | Z | 82 | −4.819 | −7.688 | 39.268 | 1.00 | 25.99 | Z | C |
| ATOM | 11975 | CB | LEU | Z | 82 | −5.377 | −7.443 | 40.668 | 1.00 | 29.09 | Z | C |
| ATOM | 11976 | CG | LEU | Z | 82 | −4.787 | −6.400 | 41.617 | 1.00 | 28.22 | Z | C |
| ATOM | 11977 | CD1 | LEU | Z | 82 | −3.421 | −5.949 | 41.139 | 1.00 | 18.10 | Z | C |
| ATOM | 11978 | CD2 | LEU | Z | 82 | −5.735 | −5.227 | 41.755 | 1.00 | 23.66 | Z | C |
| ATOM | 11979 | C | LEU | Z | 82 | −5.555 | −8.872 | 38.654 | 1.00 | 35.90 | Z | C |
| ATOM | 11980 | O | LEU | Z | 82 | −6.736 | −8.748 | 38.285 | 1.00 | 40.77 | Z | O |
| ATOM | 11981 | N | VAL | Z | 82A | −4.883 | −10.020 | 38.565 | 1.00 | 38.80 | Z | N |
| ATOM | 11982 | CA | VAL | Z | 82A | −5.473 | −11.232 | 37.974 | 1.00 | 33.54 | Z | C |
| ATOM | 11983 | CB | VAL | Z | 82A | −4.718 | −11.664 | 36.675 | 1.00 | 36.06 | Z | C |
| ATOM | 11984 | CG1 | VAL | Z | 82A | −4.833 | −10.596 | 35.600 | 1.00 | 26.42 | Z | C |
| ATOM | 11985 | CG2 | VAL | Z | 82A | −3.250 | −11.968 | 36.985 | 1.00 | 26.44 | Z | C |
| ATOM | 11986 | C | VAL | Z | 82A | −5.454 | −12.407 | 38.950 | 1.00 | 38.27 | Z | C |
| ATOM | 11987 | O | VAL | Z | 82A | −4.775 | −12.348 | 39.982 | 1.00 | 37.93 | Z | O |
| ATOM | 11988 | N | ARG | Z | 82B | −6.187 | −13.473 | 38.608 | 1.00 | 39.19 | Z | N |
| ATOM | 11989 | CA | ARG | Z | 82B | −6.289 | −14.687 | 39.434 | 1.00 | 36.16 | Z | C |
| ATOM | 11990 | CB | ARG | Z | 82B | −5.048 | −15.572 | 39.285 | 1.00 | 38.19 | Z | C |
| ATOM | 11991 | CG | ARG | Z | 82B | −4.816 | −16.083 | 37.882 | 1.00 | 43.09 | Z | C |
| ATOM | 11992 | CD | ARG | Z | 82B | −3.477 | −16.791 | 37.762 | 1.00 | 47.09 | Z | C |
| ATOM | 11993 | NE | ARG | Z | 82B | −2.883 | −16.501 | 36.461 | 1.00 | 56.48 | Z | N |
| ATOM | 11994 | CZ | ARG | Z | 82B | −1.791 | −15.764 | 36.280 | 1.00 | 58.54 | Z | C |
| ATOM | 11995 | NH1 | ARG | Z | 82B | −1.146 | −15.248 | 37.322 | 1.00 | 62.14 | Z | N |
| ATOM | 11996 | NH2 | ARG | Z | 82B | −1.381 | −15.486 | 35.050 | 1.00 | 61.34 | Z | N |
| ATOM | 11997 | C | ARG | Z | 82B | −6.497 | −14.358 | 40.903 | 1.00 | 34.95 | Z | C |
| ATOM | 11998 | O | ARG | Z | 82B | −5.849 | −14.938 | 41.776 | 1.00 | 38.19 | Z | O |
| ATOM | 11999 | N | LEU | Z | 82C | −7.419 | −13.437 | 41.163 | 1.00 | 32.24 | Z | N |
| ATOM | 12000 | CA | LEU | Z | 82C | −7.719 | −12.993 | 42.525 | 1.00 | 34.37 | Z | C |
| ATOM | 12001 | CB | LEU | Z | 82C | −8.807 | −11.927 | 42.498 | 1.00 | 30.34 | Z | C |
| ATOM | 12002 | CG | LEU | Z | 82C | −8.555 | −10.754 | 41.551 | 1.00 | 32.41 | Z | C |
| ATOM | 12003 | CD1 | LEU | Z | 82C | −9.835 | −9.964 | 41.353 | 1.00 | 26.57 | Z | C |
| ATOM | 12004 | CD2 | LEU | Z | 82C | −7.447 | −9.862 | 42.099 | 1.00 | 30.05 | Z | C |
| ATOM | 12005 | C | LEU | Z | 82C | −8.152 | −14.088 | 43.493 | 1.00 | 33.55 | Z | C |
| ATOM | 12006 | O | LEU | Z | 82C | −8.888 | −15.013 | 43.132 | 1.00 | 38.56 | Z | O |
| ATOM | 12007 | N | THR | Z | 83 | −7.629 | −13.995 | 44.712 | 1.00 | 39.43 | Z | N |
| ATOM | 12008 | CA | THR | Z | 83 | −7.955 | −14.899 | 45.814 | 1.00 | 38.16 | Z | C |
| ATOM | 12009 | CB | THR | Z | 83 | −6.735 | −15.716 | 46.309 | 1.00 | 41.12 | Z | C |
| ATOM | 12010 | OG1 | THR | Z | 83 | −5.739 | −14.836 | 46.856 | 1.00 | 44.17 | Z | O |
| ATOM | 12011 | CG2 | THR | Z | 83 | −6.136 | −16.535 | 45.167 | 1.00 | 39.90 | Z | C |
| ATOM | 12012 | C | THR | Z | 83 | −8.421 | −13.970 | 46.933 | 1.00 | 41.36 | Z | C |
| ATOM | 12013 | O | THR | Z | 83 | −8.380 | −12.746 | 46.786 | 1.00 | 42.09 | Z | O |
| ATOM | 12014 | N | SER | Z | 84 | −8.905 | −14.526 | 48.033 | 1.00 | 43.35 | Z | N |
| ATOM | 12015 | CA | SER | Z | 84 | −9.341 | −13.669 | 49.128 | 1.00 | 42.45 | Z | C |
| ATOM | 12016 | CB | SER | Z | 84 | −9.939 | −14.505 | 50.259 | 1.00 | 44.42 | Z | C |
| ATOM | 12017 | OG | SER | Z | 84 | −8.964 | −15.379 | 50.808 | 1.00 | 42.99 | Z | O |
| ATOM | 12018 | C | SER | Z | 84 | −8.147 | −12.855 | 49.644 | 1.00 | 42.92 | Z | C |
| ATOM | 12019 | O | SER | Z | 84 | −8.328 | −11.766 | 50.187 | 1.00 | 46.69 | Z | O |
| ATOM | 12020 | N | ASN | Z | 85 | −6.932 | −13.384 | 49.464 | 1.00 | 42.89 | Z | N |
| ATOM | 12021 | CA | ASN | Z | 85 | −5.699 | −12.706 | 49.895 | 1.00 | 46.17 | Z | C |
| ATOM | 12022 | CB | ASN | Z | 85 | −4.470 | −13.584 | 49.637 | 1.00 | 51.67 | Z | C |
| ATOM | 12023 | CG | ASN | Z | 85 | −4.178 | −14.539 | 50.778 | 1.00 | 60.88 | Z | C |
| ATOM | 12024 | OD1 | ASN | Z | 85 | −4.377 | −15.754 | 50.656 | 1.00 | 64.98 | Z | O |
| ATOM | 12025 | ND2 | ASN | Z | 85 | −3.673 | −14.002 | 51.888 | 1.00 | 63.06 | Z | N |
| ATOM | 12026 | C | ASN | Z | 85 | −5.477 | −11.357 | 49.198 | 1.00 | 40.77 | Z | C |
| ATOM | 12027 | O | ASN | Z | 85 | −4.660 | −10.555 | 49.635 | 1.00 | 37.08 | Z | O |
| ATOM | 12028 | N | ASP | Z | 86 | −6.209 | −11.124 | 48.116 | 1.00 | 36.69 | Z | N |
| ATOM | 12029 | CA | ASP | Z | 86 | −6.102 | −9.901 | 47.343 | 1.00 | 34.51 | Z | C |
| ATOM | 12030 | CB | ASP | Z | 86 | −6.204 | −10.212 | 45.845 | 1.00 | 33.71 | Z | C |
| ATOM | 12031 | CG | ASP | Z | 86 | −5.050 | −11.084 | 45.352 | 1.00 | 38.60 | Z | C |
| ATOM | 12032 | OD1 | ASP | Z | 86 | −3.879 | −10.770 | 45.660 | 1.00 | 41.29 | Z | O |
| ATOM | 12033 | OD2 | ASP | Z | 86 | −5.308 | −12.095 | 44.665 | 1.00 | 38.42 | Z | O |
| ATOM | 12034 | C | ASP | Z | 86 | −7.096 | −8.815 | 47.746 | 1.00 | 31.00 | Z | C |
| ATOM | 12035 | O | ASP | Z | 86 | −7.081 | −7.721 | 47.190 | 1.00 | 39.42 | Z | O |
| ATOM | 12036 | N | SER | Z | 87 | −7.978 | −9.113 | 48.687 | 1.00 | 27.86 | Z | N |
| ATOM | 12037 | CA | SER | Z | 87 | −8.919 | −8.099 | 49.157 | 1.00 | 32.78 | Z | C |
| ATOM | 12038 | CB | SER | Z | 87 | −10.023 | −8.716 | 50.025 | 1.00 | 31.44 | Z | C |
| ATOM | 12039 | OG | SER | Z | 87 | −10.865 | −9.556 | 49.262 | 1.00 | 33.33 | Z | O |
| ATOM | 12040 | C | SER | Z | 87 | −8.079 | −7.125 | 49.980 | 1.00 | 26.90 | Z | C |
| ATOM | 12041 | O | SER | Z | 87 | −7.343 | −7.539 | 50.869 | 1.00 | 30.22 | Z | O |
| ATOM | 12042 | N | ALA | Z | 88 | −8.159 | −5.847 | 49.626 | 1.00 | 27.65 | Z | N |
| ATOM | 12043 | CA | ALA | Z | 88 | −7.405 | −4.779 | 50.267 | 1.00 | 21.75 | Z | C |
| ATOM | 12044 | CB | ALA | Z | 88 | −5.939 | −5.058 | 50.151 | 1.00 | 27.69 | Z | C |
| ATOM | 12045 | C | ALA | Z | 88 | −7.718 | −3.488 | 49.543 | 1.00 | 26.38 | Z | C |
| ATOM | 12046 | O | ALA | Z | 88 | −8.462 | −3.488 | 48.566 | 1.00 | 28.10 | Z | O |
| ATOM | 12047 | N | VAL | Z | 89 | −7.187 | −2.377 | 50.047 | 1.00 | 29.20 | Z | N |
| ATOM | 12048 | CA | VAL | Z | 89 | −7.392 | −1.076 | 49.407 | 1.00 | 29.74 | Z | C |
| ATOM | 12049 | CB | VAL | Z | 89 | −7.669 | 0.028 | 50.435 | 1.00 | 29.94 | Z | C |
| ATOM | 12050 | CG1 | VAL | Z | 89 | −7.944 | 1.352 | 49.728 | 1.00 | 28.67 | Z | C |
| ATOM | 12051 | CG2 | VAL | Z | 89 | −8.864 | −0.360 | 51.288 | 1.00 | 29.01 | Z | C |
| ATOM | 12052 | C | VAL | Z | 89 | −6.120 | −0.761 | 48.618 | 1.00 | 31.44 | Z | C |
| ATOM | 12053 | O | VAL | Z | 89 | −5.014 | −0.893 | 49.136 | 1.00 | 36.42 | Z | O |
| ATOM | 12054 | N | TYR | Z | 90 | −6.280 | −0.409 | 47.348 | 1.00 | 32.08 | Z | N |
| ATOM | 12055 | CA | TYR | Z | 90 | −5.151 | −0.118 | 46.478 | 1.00 | 20.85 | Z | C |
| ATOM | 12056 | CB | TYR | Z | 90 | −5.291 | −0.891 | 45.168 | 1.00 | 18.81 | Z | C |
| ATOM | 12057 | CG | TYR | Z | 90 | −5.167 | −2.391 | 45.369 | 1.00 | 16.32 | Z | C |
| ATOM | 12058 | CD1 | TYR | Z | 90 | −6.259 | −3.162 | 45.780 | 1.00 | 14.02 | Z | C |
| ATOM | 12059 | CE1 | TYR | Z | 90 | −6.128 | −4.533 | 46.023 | 1.00 | 8.25 | Z | C |
| ATOM | 12060 | CD2 | TYR | Z | 90 | −3.949 | −3.025 | 45.203 | 1.00 | 12.01 | Z | C |
| ATOM | 12061 | CE2 | TYR | Z | 90 | −3.803 | −4.390 | 45.445 | 1.00 | 17.11 | Z | C |
| ATOM | 12062 | CZ | TYR | Z | 90 | −4.895 | −5.142 | 45.856 | 1.00 | 18.28 | Z | C |
| ATOM | 12063 | OH | TYR | Z | 90 | −4.729 | −6.503 | 46.098 | 1.00 | 18.90 | Z | O |
| ATOM | 12064 | C | TYR | Z | 90 | −5.013 | 1.361 | 46.226 | 1.00 | 23.00 | Z | C |
| ATOM | 12065 | O | TYR | Z | 90 | −5.989 | 2.035 | 45.895 | 1.00 | 18.04 | Z | O |
| ATOM | 12066 | N | TYR | Z | 91 | −3.799 | 1.864 | 46.454 | 1.00 | 23.35 | Z | N |
| ATOM | 12067 | CA | TYR | Z | 91 | −3.489 | 3.276 | 46.271 | 1.00 | 21.10 | Z | C |
| ATOM | 12068 | CB | TYR | Z | 91 | −2.915 | 3.906 | 47.553 | 1.00 | 22.88 | Z | C |
| ATOM | 12069 | CG | TYR | Z | 91 | −3.828 | 3.929 | 48.755 | 1.00 | 27.31 | Z | C |
| ATOM | 12070 | CD1 | TYR | Z | 91 | −3.895 | 2.845 | 49.617 | 1.00 | 24.21 | Z | C |
| ATOM | 12071 | CE1 | TYR | Z | 91 | −4.723 | 2.858 | 50.716 | 1.00 | 33.73 | Z | C |
| ATOM | 12072 | CD2 | TYR | Z | 91 | −4.620 | 5.039 | 49.030 | 1.00 | 28.49 | Z | C |
| ATOM | 12073 | CE2 | TYR | Z | 91 | −5.462 | 5.062 | 50.145 | 1.00 | 32.08 | Z | C |
| ATOM | 12074 | CZ | TYR | Z | 91 | −5.505 | 3.967 | 50.978 | 1.00 | 29.03 | Z | C |
| ATOM | 12075 | OH | TYR | Z | 91 | −6.329 | 3.963 | 52.074 | 1.00 | 34.00 | Z | O |
| ATOM | 12076 | C | TYR | Z | 91 | −2.458 | 3.471 | 45.189 | 1.00 | 21.93 | Z | C |
| ATOM | 12077 | O | TYR | Z | 91 | −1.524 | 2.681 | 45.028 | 1.00 | 25.31 | Z | O |
| ATOM | 12078 | N | CYS | Z | 92 | −2.623 | 4.582 | 44.501 | 1.00 | 21.69 | Z | N |
| ATOM | 12079 | CA | CYS | Z | 92 | −1.739 | 5.026 | 43.443 | 1.00 | 31.39 | Z | C |
| ATOM | 12080 | C | CYS | Z | 92 | −1.050 | 6.254 | 44.073 | 1.00 | 31.41 | Z | C |
| ATOM | 12081 | O | CYS | Z | 92 | −1.703 | 7.090 | 44.717 | 1.00 | 25.69 | Z | O |
| ATOM | 12082 | CB | CYS | Z | 92 | −2.595 | 5.360 | 42.211 | 1.00 | 29.77 | Z | C |
| ATOM | 12083 | SG | CYS | Z | 92 | −2.172 | 6.774 | 41.164 | 0.50 | 36.56 | Z | S |
| ATOM | 12084 | N | ALA | Z | 93 | 0.273 | 6.311 | 43.975 | 1.00 | 27.51 | Z | N |
| ATOM | 12085 | CA | ALA | Z | 93 | 1.013 | 7.418 | 44.580 | 1.00 | 25.94 | Z | C |
| ATOM | 12086 | CB | ALA | Z | 93 | 1.556 | 7.010 | 45.926 | 1.00 | 18.85 | Z | C |
| ATOM | 12087 | C | ALA | Z | 93 | 2.125 | 7.984 | 43.716 | 1.00 | 22.04 | Z | C |
| ATOM | 12088 | O | ALA | Z | 93 | 2.971 | 7.257 | 43.196 | 1.00 | 23.38 | Z | O |
| ATOM | 12089 | N | ARG | Z | 94 | 2.108 | 9.304 | 43.608 | 1.00 | 15.86 | Z | N |
| ATOM | 12090 | CA | ARG | Z | 94 | 3.061 | 10.068 | 42.842 | 1.00 | 18.51 | Z | C |
| ATOM | 12091 | CB | ARG | Z | 94 | 2.459 | 11.431 | 42.605 | 1.00 | 21.70 | Z | C |
| ATOM | 12092 | CG | ARG | Z | 94 | 3.208 | 12.305 | 41.674 | 1.00 | 18.89 | Z | C |
| ATOM | 12093 | CD | ARG | Z | 94 | 3.140 | 13.669 | 42.268 | 1.00 | 23.45 | Z | C |
| ATOM | 12094 | NE | ARG | Z | 94 | 2.876 | 14.663 | 41.266 | 1.00 | 30.10 | Z | N |
| ATOM | 12095 | CZ | ARG | Z | 94 | 3.084 | 15.951 | 41.447 | 1.00 | 31.32 | Z | C |
| ATOM | 12096 | NH1 | ARG | Z | 94 | 3.570 | 16.392 | 42.600 | 1.00 | 27.74 | Z | N |
| ATOM | 12097 | NH2 | ARG | Z | 94 | 2.748 | 16.792 | 40.485 | 1.00 | 36.05 | Z | N |
| ATOM | 12098 | C | ARG | Z | 94 | 4.406 | 10.192 | 43.570 | 1.00 | 19.79 | Z | C |
| ATOM | 12099 | O | ARG | Z | 94 | 4.469 | 10.238 | 44.789 | 1.00 | 26.49 | Z | O |
| ATOM | 12100 | N | GLU | Z | 95 | 5.489 | 10.195 | 42.810 | 1.00 | 24.18 | Z | N |
| ATOM | 12101 | CA | GLU | Z | 95 | 6.826 | 10.292 | 43.374 | 1.00 | 20.32 | Z | C |
| ATOM | 12102 | CB | GLU | Z | 95 | 7.465 | 8.915 | 43.465 | 1.00 | 20.82 | Z | C |
| ATOM | 12103 | CG | GLU | Z | 95 | 8.799 | 8.887 | 44.200 | 1.00 | 21.06 | Z | C |
| ATOM | 12104 | CD | GLU | Z | 95 | 9.390 | 7.501 | 44.222 | 1.00 | 26.52 | Z | C |
| ATOM | 12105 | OE1 | GLU | Z | 95 | 8.645 | 6.543 | 43.950 | 1.00 | 30.41 | Z | O |
| ATOM | 12106 | OE2 | GLU | Z | 95 | 10.597 | 7.355 | 44.494 | 1.00 | 36.22 | Z | O |
| ATOM | 12107 | C | GLU | Z | 95 | 7.706 | 11.177 | 42.524 | 1.00 | 24.44 | Z | C |
| ATOM | 12108 | O | GLU | Z | 95 | 7.852 | 10.959 | 41.309 | 1.00 | 23.70 | Z | O |
| ATOM | 12109 | N | ASP | Z | 96 | 8.270 | 12.191 | 43.171 | 1.00 | 25.14 | Z | N |
| ATOM | 12110 | CA | ASP | Z | 96 | 9.166 | 13.134 | 42.517 | 1.00 | 18.09 | Z | C |
| ATOM | 12111 | CB | ASP | Z | 96 | 9.258 | 14.410 | 43.351 | 1.00 | 17.98 | Z | C |
| ATOM | 12112 | CG | ASP | Z | 96 | 10.432 | 15.289 | 42.951 | 1.00 | 27.30 | Z | C |
| ATOM | 12113 | OD1 | ASP | Z | 96 | 11.515 | 15.159 | 43.558 | 1.00 | 25.29 | Z | O |
| ATOM | 12114 | OD2 | ASP | Z | 96 | 10.279 | 16.107 | 42.024 | 1.00 | 30.43 | Z | O |
| ATOM | 12115 | C | ASP | Z | 96 | 10.556 | 12.513 | 42.348 | 1.00 | 18.68 | Z | C |
| ATOM | 12116 | O | ASP | Z | 96 | 10.939 | 11.603 | 43.091 | 1.00 | 14.15 | Z | O |
| ATOM | 12117 | N | PHE | Z | 97 | 11.291 | 12.954 | 41.335 | 1.00 | 13.59 | Z | N |
| ATOM | 12118 | CA | PHE | Z | 97 | 12.640 | 12.433 | 41.147 | 1.00 | 20.88 | Z | C |
| ATOM | 12119 | CB | PHE | Z | 97 | 12.708 | 11.225 | 40.166 | 1.00 | 15.35 | Z | C |
| ATOM | 12120 | CG | PHE | Z | 97 | 12.074 | 11.466 | 38.827 | 1.00 | 17.33 | Z | C |
| ATOM | 12121 | CD1 | PHE | Z | 97 | 12.119 | 12.711 | 38.222 | 1.00 | 15.86 | Z | C |
| ATOM | 12122 | CD2 | PHE | Z | 97 | 11.420 | 10.432 | 38.170 | 1.00 | 22.03 | Z | C |
| ATOM | 12123 | CE1 | PHE | Z | 97 | 11.525 | 12.924 | 36.986 | 1.00 | 16.90 | Z | C |
| ATOM | 12124 | CE2 | PHE | Z | 97 | 10.820 | 10.639 | 36.932 | 1.00 | 19.10 | Z | C |
| ATOM | 12125 | CZ | PHE | Z | 97 | 10.876 | 11.889 | 36.342 | 1.00 | 18.87 | Z | C |
| ATOM | 12126 | C | PHE | Z | 97 | 13.609 | 13.540 | 40.776 | 1.00 | 17.60 | Z | C |
| ATOM | 12127 | O | PHE | Z | 97 | 14.677 | 13.286 | 40.251 | 1.00 | 16.20 | Z | O |
| ATOM | 12128 | N | ARG | Z | 98 | 13.226 | 14.771 | 41.092 | 1.00 | 23.55 | Z | N |
| ATOM | 12129 | CA | ARG | Z | 98 | 14.048 | 15.936 | 40.803 | 1.00 | 21.22 | Z | C |
| ATOM | 12130 | CB | ARG | Z | 98 | 13.207 | 17.013 | 40.114 | 1.00 | 19.90 | Z | C |
| ATOM | 12131 | CG | ARG | Z | 98 | 12.782 | 16.658 | 38.666 | 1.00 | 21.31 | Z | C |
| ATOM | 12132 | CD | ARG | Z | 98 | 12.244 | 17.898 | 37.957 | 1.00 | 17.34 | Z | C |
| ATOM | 12133 | NE | ARG | Z | 98 | 11.688 | 17.632 | 36.638 | 1.00 | 24.86 | Z | N |
| ATOM | 12134 | CZ | ARG | Z | 98 | 10.527 | 17.012 | 36.413 | 1.00 | 28.11 | Z | C |
| ATOM | 12135 | NH1 | ARG | Z | 98 | 9.775 | 16.584 | 37.425 | 1.00 | 25.96 | Z | N |
| ATOM | 12136 | NH2 | ARG | Z | 98 | 10.123 | 16.806 | 35.168 | 1.00 | 25.54 | Z | N |
| ATOM | 12137 | C | ARG | Z | 98 | 14.811 | 16.540 | 41.994 | 1.00 | 26.81 | Z | C |
| ATOM | 12138 | O | ARG | Z | 98 | 15.982 | 16.918 | 41.856 | 1.00 | 26.02 | Z | O |
| ATOM | 12139 | N | TYR | Z | 99 | 14.160 | 16.651 | 43.152 | 1.00 | 24.80 | Z | N |
| ATOM | 12140 | CA | TYR | Z | 99 | 14.815 | 17.249 | 44.312 | 1.00 | 29.28 | Z | C |
| ATOM | 12141 | CB | TYR | Z | 99 | 14.834 | 18.775 | 44.171 | 1.00 | 21.32 | Z | C |
| ATOM | 12142 | CG | TYR | Z | 99 | 13.483 | 19.447 | 44.120 | 1.00 | 22.34 | Z | C |
| ATOM | 12143 | CD1 | TYR | Z | 99 | 12.842 | 19.678 | 42.906 | 1.00 | 19.05 | Z | C |
| ATOM | 12144 | CE1 | TYR | Z | 99 | 11.655 | 20.394 | 42.853 | 1.00 | 20.68 | Z | C |
| ATOM | 12145 | CD2 | TYR | Z | 99 | 12.885 | 19.943 | 45.292 | 1.00 | 21.89 | Z | C |
| ATOM | 12146 | CE2 | TYR | Z | 99 | 11.699 | 20.660 | 45.251 | 1.00 | 14.02 | Z | C |
| ATOM | 12147 | CZ | TYR | Z | 99 | 11.093 | 20.889 | 44.027 | 1.00 | 22.65 | Z | C |
| ATOM | 12148 | OH | TYR | Z | 99 | 9.957 | 21.658 | 43.975 | 1.00 | 21.30 | Z | O |
| ATOM | 12149 | C | TYR | Z | 99 | 14.351 | 16.831 | 45.719 | 1.00 | 29.28 | Z | C |
| ATOM | 12150 | O | TYR | Z | 99 | 15.138 | 16.879 | 46.659 | 1.00 | 34.01 | Z | O |
| ATOM | 12151 | N | HIS | Z | 100 | 13.071 | 16.494 | 45.870 | 1.00 | 34.04 | Z | N |
| ATOM | 12152 | CA | HIS | Z | 100 | 12.496 | 16.022 | 47.146 | 1.00 | 30.73 | Z | C |
| ATOM | 12153 | CB | HIS | Z | 100 | 11.379 | 16.957 | 47.625 | 1.00 | 21.93 | Z | C |
| ATOM | 12154 | CG | HIS | Z | 100 | 10.153 | 16.918 | 46.768 | 1.00 | 22.69 | Z | C |
| ATOM | 12155 | CD2 | HIS | Z | 100 | 9.799 | 17.645 | 45.682 | 1.00 | 22.49 | Z | C |
| ATOM | 12156 | ND1 | HIS | Z | 100 | 9.146 | 15.991 | 46.956 | 1.00 | 17.92 | Z | N |
| ATOM | 12157 | CE1 | HIS | Z | 100 | 8.227 | 16.152 | 46.022 | 1.00 | 26.66 | Z | C |
| ATOM | 12158 | NE2 | HIS | Z | 100 | 8.597 | 17.150 | 45.237 | 1.00 | 24.66 | Z | N |
| ATOM | 12159 | C | HIS | Z | 100 | 11.937 | 14.691 | 46.665 | 1.00 | 36.99 | Z | C |
| ATOM | 12160 | O | HIS | Z | 100 | 10.909 | 14.610 | 45.995 | 1.00 | 51.19 | Z | O |
| ATOM | 12161 | N | TYR | Z | 100A | 12.634 | 13.620 | 46.949 | 1.00 | 38.70 | Z | N |
| ATOM | 12162 | CA | TYR | Z | 100A | 12.193 | 12.348 | 46.405 | 1.00 | 27.38 | Z | C |
| ATOM | 12163 | CB | TYR | Z | 100A | 13.430 | 11.481 | 46.233 | 1.00 | 28.37 | Z | C |
| ATOM | 12164 | CG | TYR | Z | 100A | 14.449 | 12.203 | 45.369 | 1.00 | 22.36 | Z | C |
| ATOM | 12165 | CD1 | TYR | Z | 100A | 15.168 | 13.304 | 45.867 | 1.00 | 17.13 | Z | C |
| ATOM | 12166 | CE1 | TYR | Z | 100A | 16.043 | 14.025 | 45.055 | 1.00 | 16.82 | Z | C |
| ATOM | 12167 | CD2 | TYR | Z | 100A | 14.645 | 11.839 | 44.039 | 1.00 | 15.25 | Z | C |
| ATOM | 12168 | CE2 | TYR | Z | 100A | 15.523 | 12.547 | 43.224 | 1.00 | 24.23 | Z | C |
| ATOM | 12169 | CZ | TYR | Z | 100A | 16.214 | 13.646 | 43.733 | 1.00 | 23.74 | Z | C |
| ATOM | 12170 | OH | TYR | Z | 100A | 17.005 | 14.398 | 42.894 | 1.00 | 17.17 | Z | O |
| ATOM | 12171 | C | TYR | Z | 100A | 11.066 | 11.683 | 47.162 | 1.00 | 27.24 | Z | C |
| ATOM | 12172 | O | TYR | Z | 100A | 11.085 | 10.478 | 47.403 | 1.00 | 27.59 | Z | O |
| ATOM | 12173 | N | SER | Z | 100B | 10.042 | 12.479 | 47.459 | 1.00 | 20.91 | Z | N |
| ATOM | 12174 | CA | SER | Z | 100B | 8.891 | 12.009 | 48.212 | 1.00 | 25.63 | Z | C |
| ATOM | 12175 | CB | SER | Z | 100B | 8.448 | 13.069 | 49.215 | 1.00 | 26.09 | Z | C |
| ATOM | 12176 | OG | SER | Z | 100B | 7.948 | 14.204 | 48.556 | 1.00 | 32.80 | Z | O |
| ATOM | 12177 | C | SER | Z | 100B | 7.661 | 11.574 | 47.437 | 1.00 | 23.14 | Z | C |
| ATOM | 12178 | O | SER | Z | 100B | 7.441 | 11.966 | 46.283 | 1.00 | 26.37 | Z | O |
| ATOM | 12179 | N | MET | Z | 100C | 6.894 | 10.707 | 48.085 | 1.00 | 19.20 | Z | N |
| ATOM | 12180 | CA | MET | Z | 100C | 5.626 | 10.234 | 47.561 | 1.00 | 16.57 | Z | C |
| ATOM | 12181 | CB | MET | Z | 100C | 5.319 | 8.837 | 48.084 | 1.00 | 11.49 | Z | C |
| ATOM | 12182 | CG | MET | Z | 100C | 6.067 | 7.777 | 47.311 | 1.00 | 14.83 | Z | C |
| ATOM | 12183 | SD | MET | Z | 100C | 5.967 | 6.174 | 48.052 | 1.00 | 32.56 | Z | S |
| ATOM | 12184 | CE | MET | Z | 100C | 7.186 | 5.317 | 47.069 | 1.00 | 27.40 | Z | C |
| ATOM | 12185 | C | MET | Z | 100C | 4.656 | 11.270 | 48.109 | 1.00 | 19.54 | Z | C |
| ATOM | 12186 | O | MET | Z | 100C | 4.074 | 11.091 | 49.187 | 1.00 | 17.36 | Z | O |
| ATOM | 12187 | N | ASP | Z | 101 | 4.566 | 12.392 | 47.395 | 1.00 | 14.71 | Z | N |
| ATOM | 12188 | CA | ASP | Z | 101 | 3.737 | 13.528 | 47.809 | 1.00 | 25.80 | Z | C |
| ATOM | 12189 | CB | ASP | Z | 101 | 4.209 | 14.842 | 47.142 | 1.00 | 20.36 | Z | C |
| ATOM | 12190 | CG | ASP | Z | 101 | 4.430 | 14.707 | 45.627 | 1.00 | 29.95 | Z | C |
| ATOM | 12191 | OD1 | ASP | Z | 101 | 4.182 | 13.613 | 45.074 | 1.00 | 24.89 | Z | O |
| ATOM | 12192 | OD2 | ASP | Z | 101 | 4.875 | 15.697 | 44.990 | 1.00 | 23.20 | Z | O |
| ATOM | 12193 | C | ASP | Z | 101 | 2.225 | 13.393 | 47.707 | 1.00 | 25.26 | Z | C |
| ATOM | 12194 | O | ASP | Z | 101 | 1.548 | 13.507 | 48.710 | 1.00 | 27.03 | Z | O |
| ATOM | 12195 | N | TYR | Z | 102 | 1.699 | 13.191 | 46.501 | 1.00 | 27.27 | Z | N |
| ATOM | 12196 | CA | TYR | Z | 102 | 0.261 | 13.058 | 46.330 | 1.00 | 22.26 | Z | C |
| ATOM | 12197 | CB | TYR | Z | 102 | −0.243 | 13.921 | 45.188 | 1.00 | 18.95 | Z | C |
| ATOM | 12198 | CG | TYR | Z | 102 | −0.150 | 15.404 | 45.457 | 1.00 | 24.88 | Z | C |
| ATOM | 12199 | CD1 | TYR | Z | 102 | 1.007 | 16.124 | 45.147 | 1.00 | 26.78 | Z | C |
| ATOM | 12200 | CE1 | TYR | Z | 102 | 1.084 | 17.492 | 45.383 | 1.00 | 25.32 | Z | C |
| ATOM | 12201 | CD2 | TYR | Z | 102 | −1.222 | 16.095 | 46.006 | 1.00 | 23.04 | Z | C |
| ATOM | 12202 | CE2 | TYR | Z | 102 | −1.156 | 17.461 | 46.243 | 1.00 | 25.25 | Z | C |
| ATOM | 12203 | CZ | TYR | Z | 102 | −0.007 | 18.153 | 45.932 | 1.00 | 27.00 | Z | C |
| ATOM | 12204 | OH | TYR | Z | 102 | 0.040 | 19.507 | 46.170 | 1.00 | 30.62 | Z | O |
| ATOM | 12205 | C | TYR | Z | 102 | −0.197 | 11.630 | 46.142 | 1.00 | 24.58 | Z | C |
| ATOM | 12206 | O | TYR | Z | 102 | 0.362 | 10.869 | 45.347 | 1.00 | 25.79 | Z | O |
| ATOM | 12207 | N | TRP | Z | 103 | −1.213 | 11.275 | 46.918 | 1.00 | 26.79 | Z | N |
| ATOM | 12208 | CA | TRP | Z | 103 | −1.807 | 9.952 | 46.895 | 1.00 | 25.13 | Z | C |
| ATOM | 12209 | CB | TRP | Z | 103 | −1.749 | 9.343 | 48.296 | 1.00 | 26.02 | Z | C |
| ATOM | 12210 | CG | TRP | Z | 103 | −0.385 | 8.897 | 48.709 | 1.00 | 19.04 | Z | C |
| ATOM | 12211 | CD2 | TRP | Z | 103 | −0.002 | 7.571 | 49.080 | 1.00 | 16.16 | Z | C |
| ATOM | 12212 | CE2 | TRP | Z | 103 | 1.381 | 7.593 | 49.335 | 1.00 | 15.64 | Z | C |
| ATOM | 12213 | CE3 | TRP | Z | 103 | −0.700 | 6.366 | 49.225 | 1.00 | 17.46 | Z | C |
| ATOM | 12214 | CD1 | TRP | Z | 103 | 0.744 | 9.654 | 48.767 | 1.00 | 16.61 | Z | C |
| ATOM | 12215 | NE1 | TRP | Z | 103 | 1.813 | 8.877 | 49.135 | 1.00 | 15.09 | Z | N |
| ATOM | 12216 | CZ2 | TRP | Z | 103 | 2.088 | 6.458 | 49.726 | 1.00 | 15.17 | Z | C |
| ATOM | 12217 | CZ3 | TRP | Z | 103 | −0.007 | 5.243 | 49.612 | 1.00 | 23.25 | Z | C |
| ATOM | 12218 | CH2 | TRP | Z | 103 | 1.384 | 5.294 | 49.860 | 1.00 | 27.00 | Z | C |
| ATOM | 12219 | C | TRP | Z | 103 | −3.264 | 9.994 | 46.421 | 1.00 | 27.36 | Z | C |
| ATOM | 12220 | O | TRP | Z | 103 | −3.957 | 11.008 | 46.582 | 1.00 | 24.95 | Z | O |
| ATOM | 12221 | N | GLY | Z | 104 | −3.694 | 8.914 | 45.769 | 1.00 | 26.57 | Z | N |
| ATOM | 12222 | CA | GLY | Z | 104 | −5.070 | 8.817 | 45.320 | 1.00 | 25.35 | Z | C |
| ATOM | 12223 | C | GLY | Z | 104 | −5.883 | 8.465 | 46.552 | 1.00 | 28.42 | Z | C |
| ATOM | 12224 | O | GLY | Z | 104 | −5.298 | 8.049 | 47.561 | 1.00 | 30.41 | Z | O |
| ATOM | 12225 | N | GLN | Z | 105 | −7.210 | 8.606 | 46.489 | 1.00 | 29.08 | Z | N |
| ATOM | 12226 | CA | GLN | Z | 105 | −8.069 | 8.299 | 47.640 | 1.00 | 21.99 | Z | C |
| ATOM | 12227 | CB | GLN | Z | 105 | −9.488 | 8.855 | 47.451 | 1.00 | 27.03 | Z | C |
| ATOM | 12228 | CG | GLN | Z | 105 | −10.442 | 7.972 | 46.636 | 1.00 | 29.92 | Z | C |
| ATOM | 12229 | CD | GLN | Z | 105 | −10.246 | 8.088 | 45.133 | 1.00 | 32.24 | Z | C |
| ATOM | 12230 | OE1 | GLN | Z | 105 | −9.214 | 8.556 | 44.658 | 1.00 | 30.31 | Z | O |
| ATOM | 12231 | NE2 | GLN | Z | 105 | −11.250 | 7.670 | 44.379 | 1.00 | 32.01 | Z | N |
| ATOM | 12232 | C | GLN | Z | 105 | −8.134 | 6.814 | 47.952 | 1.00 | 22.96 | Z | C |
| ATOM | 12233 | O | GLN | Z | 105 | −8.576 | 6.417 | 49.023 | 1.00 | 29.89 | Z | O |
| ATOM | 12234 | N | GLY | Z | 106 | −7.699 | 5.990 | 47.012 | 1.00 | 23.63 | Z | N |
| ATOM | 12235 | CA | GLY | Z | 106 | −7.727 | 4.558 | 47.230 | 1.00 | 17.45 | Z | C |
| ATOM | 12236 | C | GLY | Z | 106 | −8.952 | 3.918 | 46.597 | 1.00 | 21.62 | Z | C |
| ATOM | 12237 | O | GLY | Z | 106 | −9.979 | 4.573 | 46.407 | 1.00 | 16.76 | Z | O |
| ATOM | 12238 | N | THR | Z | 107 | −8.815 | 2.641 | 46.249 | 1.00 | 17.88 | Z | N |
| ATOM | 12239 | CA | THR | Z | 107 | −9.884 | 1.867 | 45.628 | 1.00 | 29.11 | Z | C |
| ATOM | 12240 | CB | THR | Z | 107 | −9.554 | 1.515 | 44.145 | 1.00 | 29.80 | Z | C |
| ATOM | 12241 | OG1 | THR | Z | 107 | −9.531 | 2.706 | 43.361 | 1.00 | 27.52 | Z | O |
| ATOM | 12242 | CG2 | THR | Z | 107 | −10.604 | 0.566 | 43.555 | 1.00 | 33.34 | Z | C |
| ATOM | 12243 | C | THR | Z | 107 | −10.053 | 0.573 | 46.411 | 1.00 | 28.27 | Z | C |
| ATOM | 12244 | O | THR | Z | 107 | −9.104 | −0.211 | 46.533 | 1.00 | 26.01 | Z | O |
| ATOM | 12245 | N | SER | Z | 108 | −11.258 | 0.352 | 46.932 | 1.00 | 22.04 | Z | N |
| ATOM | 12246 | CA | SER | Z | 108 | −11.529 | −0.849 | 47.712 | 1.00 | 28.94 | Z | C |
| ATOM | 12247 | CB | SER | Z | 108 | −12.675 | −0.599 | 48.696 | 1.00 | 28.72 | Z | C |
| ATOM | 12248 | OG | SER | Z | 108 | −12.830 | −1.699 | 49.568 | 1.00 | 30.86 | Z | O |
| ATOM | 12249 | C | SER | Z | 108 | −11.845 | −2.067 | 46.844 | 1.00 | 26.67 | Z | C |
| ATOM | 12250 | O | SER | Z | 108 | −12.757 | −2.051 | 46.015 | 1.00 | 29.69 | Z | O |
| ATOM | 12251 | N | VAL | Z | 109 | −11.073 | −3.123 | 47.031 | 1.00 | 26.94 | Z | N |
| ATOM | 12252 | CA | VAL | Z | 109 | −11.294 | −4.350 | 46.285 | 1.00 | 30.30 | Z | C |
| ATOM | 12253 | CB | VAL | Z | 109 | −10.006 | −4.790 | 45.515 | 1.00 | 32.57 | Z | C |
| ATOM | 12254 | CG1 | VAL | Z | 109 | −10.184 | −6.195 | 44.921 | 1.00 | 36.63 | Z | C |
| ATOM | 12255 | CG2 | VAL | Z | 109 | −9.703 | −3.814 | 44.395 | 1.00 | 32.70 | Z | C |
| ATOM | 12256 | C | VAL | Z | 109 | −11.722 | −5.482 | 47.225 | 1.00 | 28.01 | Z | C |
| ATOM | 12257 | O | VAL | Z | 109 | −11.012 | −5.808 | 48.181 | 1.00 | 27.17 | Z | O |
| ATOM | 12258 | N | THR | Z | 110 | −12.900 | −6.048 | 46.980 | 1.00 | 30.06 | Z | N |
| ATOM | 12259 | CA | THR | Z | 110 | −13.366 | −7.165 | 47.779 | 1.00 | 25.25 | Z | C |
| ATOM | 12260 | CB | THR | Z | 110 | −14.593 | −6.830 | 48.593 | 1.00 | 27.12 | Z | C |
| ATOM | 12261 | OG1 | THR | Z | 110 | −14.397 | −5.573 | 49.246 | 1.00 | 31.27 | Z | O |
| ATOM | 12262 | CG2 | THR | Z | 110 | −14.772 | −7.874 | 49.657 | 1.00 | 30.30 | Z | C |
| ATOM | 12263 | C | THR | Z | 110 | −13.592 | −8.375 | 46.873 | 1.00 | 29.26 | Z | C |
| ATOM | 12264 | O | THR | Z | 110 | −14.304 | −8.315 | 45.869 | 1.00 | 29.17 | Z | O |
| ATOM | 12265 | N | VAL | Z | 111 | −12.843 | −9.426 | 47.184 | 1.00 | 28.30 | Z | N |
| ATOM | 12266 | CA | VAL | Z | 111 | −12.858 | −10.682 | 46.461 | 1.00 | 32.31 | Z | C |
| ATOM | 12267 | CB | VAL | Z | 111 | −11.418 | −11.141 | 46.131 | 1.00 | 35.61 | Z | C |
| ATOM | 12268 | CG1 | VAL | Z | 111 | −11.459 | −12.447 | 45.332 | 1.00 | 31.33 | Z | C |
| ATOM | 12269 | CG2 | VAL | Z | 111 | −10.641 | −10.032 | 45.388 | 1.00 | 25.20 | Z | C |
| ATOM | 12270 | C | VAL | Z | 111 | −13.448 | −11.737 | 47.383 | 1.00 | 35.09 | Z | C |
| ATOM | 12271 | O | VAL | Z | 111 | −12.819 | −12.113 | 48.363 | 1.00 | 37.07 | Z | O |
| ATOM | 12272 | N | SER | Z | 112 | −14.612 | −12.270 | 47.023 | 1.00 | 36.23 | Z | N |
| ATOM | 12273 | CA | SER | Z | 112 | −15.291 | −13.266 | 47.845 | 1.00 | 29.74 | Z | C |
| ATOM | 12274 | CB | SER | Z | 112 | −16.118 | −12.529 | 48.918 | 1.00 | 24.37 | Z | C |
| ATOM | 12275 | OG | SER | Z | 112 | −16.860 | −13.406 | 49.739 | 1.00 | 26.80 | Z | O |
| ATOM | 12276 | C | SER | Z | 112 | −16.208 | −14.131 | 46.972 | 1.00 | 30.52 | Z | C |
| ATOM | 12277 | O | SER | Z | 112 | −16.699 | −13.676 | 45.935 | 1.00 | 26.74 | Z | O |
| ATOM | 12278 | N | SER | Z | 113 | −16.395 | −15.389 | 47.373 | 1.00 | 30.72 | Z | N |
| ATOM | 12279 | CA | SER | Z | 113 | −17.284 | −16.306 | 46.658 | 1.00 | 31.72 | Z | C |
| ATOM | 12280 | CB | SER | Z | 113 | −16.628 | −17.679 | 46.470 | 1.00 | 33.75 | Z | C |
| ATOM | 12281 | OG | SER | Z | 113 | −16.232 | −18.243 | 47.703 | 1.00 | 35.59 | Z | O |
| ATOM | 12282 | C | SER | Z | 113 | −18.594 | −16.424 | 47.453 | 1.00 | 36.54 | Z | C |
| ATOM | 12283 | O | SER | Z | 113 | −19.416 | −17.329 | 47.238 | 1.00 | 36.78 | Z | O |
| ATOM | 12284 | N | ALA | Z | 114 | −18.774 | −15.490 | 48.378 | 1.00 | 32.20 | Z | N |
| ATOM | 12285 | CA | ALA | Z | 114 | −19.953 | −15.470 | 49.209 | 1.00 | 37.80 | Z | C |
| ATOM | 12286 | CB | ALA | Z | 114 | −19.705 | −14.623 | 50.461 | 1.00 | 34.43 | Z | C |
| ATOM | 12287 | C | ALA | Z | 114 | −21.135 | −14.926 | 48.421 | 1.00 | 39.37 | Z | C |
| ATOM | 12288 | O | ALA | Z | 114 | −20.983 | −14.061 | 47.549 | 1.00 | 41.26 | Z | O |
| ATOM | 12289 | N | LYS | Z | 115 | −22.308 | −15.458 | 48.737 | 1.00 | 37.35 | Z | N |
| ATOM | 12290 | CA | LYS | Z | 115 | −23.551 | −15.048 | 48.104 | 1.00 | 38.16 | Z | C |
| ATOM | 12291 | CB | LYS | Z | 115 | −24.393 | −16.300 | 47.786 | 1.00 | 40.37 | Z | C |
| ATOM | 12292 | CG | LYS | Z | 115 | −24.571 | −17.252 | 48.954 | 1.00 | 45.03 | Z | C |
| ATOM | 12293 | CD | LYS | Z | 115 | −25.223 | −18.556 | 48.530 | 1.00 | 46.78 | Z | C |
| ATOM | 12294 | CE | LYS | Z | 115 | −25.696 | −19.338 | 49.748 | 1.00 | 51.29 | Z | C |
| ATOM | 12295 | NZ | LYS | Z | 115 | −26.727 | −18.557 | 50.517 | 1.00 | 48.96 | Z | N |
| ATOM | 12296 | C | LYS | Z | 115 | −24.301 | −14.104 | 49.048 | 1.00 | 34.95 | Z | C |
| ATOM | 12297 | O | LYS | Z | 115 | −24.010 | −14.062 | 50.242 | 1.00 | 34.47 | Z | O |
| ATOM | 12298 | N | THR | Z | 116 | −25.254 | −13.345 | 48.519 | 1.00 | 28.99 | Z | N |
| ATOM | 12299 | CA | THR | Z | 116 | −26.021 | −12.431 | 49.342 | 1.00 | 23.28 | Z | C |
| ATOM | 12300 | CB | THR | Z | 116 | −27.014 | −11.634 | 48.497 | 1.00 | 22.00 | Z | C |
| ATOM | 12301 | OG1 | THR | Z | 116 | −26.295 | −10.854 | 47.538 | 1.00 | 26.09 | Z | O |
| ATOM | 12302 | CG2 | THR | Z | 116 | −27.864 | −10.703 | 49.367 | 1.00 | 22.65 | Z | C |
| ATOM | 12303 | C | THR | Z | 116 | −26.769 | −13.210 | 50.417 | 1.00 | 30.24 | Z | C |
| ATOM | 12304 | O | THR | Z | 116 | −27.587 | −14.080 | 50.113 | 1.00 | 34.50 | Z | O |
| ATOM | 12305 | N | THR | Z | 117 | −26.431 | −12.949 | 51.677 | 1.00 | 29.02 | Z | N |
| ATOM | 12306 | CA | THR | Z | 117 | −27.088 | −13.618 | 52.785 | 1.00 | 29.66 | Z | C |
| ATOM | 12307 | CB | THR | Z | 117 | −26.299 | −14.886 | 53.250 | 1.00 | 30.90 | Z | C |
| ATOM | 12308 | OG1 | THR | Z | 117 | −26.273 | −14.987 | 54.679 | 1.00 | 27.66 | Z | O |
| ATOM | 12309 | CG2 | THR | Z | 117 | −24.928 | −14.890 | 52.713 | 1.00 | 27.34 | Z | C |
| ATOM | 12310 | C | THR | Z | 117 | −27.396 | −12.643 | 53.917 | 1.00 | 28.32 | Z | C |
| ATOM | 12311 | O | THR | Z | 117 | −26.560 | −11.826 | 54.297 | 1.00 | 36.08 | Z | O |
| ATOM | 12312 | N | PRO | Z | 118 | −28.640 | −12.666 | 54.418 | 1.00 | 25.44 | Z | N |
| ATOM | 12313 | CD | PRO | Z | 118 | −29.747 | −13.561 | 54.021 | 1.00 | 20.19 | Z | C |
| ATOM | 12314 | CA | PRO | Z | 118 | −29.045 | −11.771 | 55.507 | 1.00 | 18.37 | Z | C |
| ATOM | 12315 | CB | PRO | Z | 118 | −30.567 | −11.909 | 55.506 | 1.00 | 19.99 | Z | C |
| ATOM | 12316 | CG | PRO | Z | 118 | −30.747 | −13.368 | 55.158 | 1.00 | 19.24 | Z | C |
| ATOM | 12317 | C | PRO | Z | 118 | −28.459 | −12.213 | 56.842 | 1.00 | 19.58 | Z | C |
| ATOM | 12318 | O | PRO | Z | 118 | −28.083 | −13.382 | 57.014 | 1.00 | 24.99 | Z | O |
| ATOM | 12319 | N | PRO | Z | 119 | −28.420 | −11.300 | 57.828 | 1.00 | 17.86 | Z | N |
| ATOM | 12320 | CD | PRO | Z | 119 | −28.684 | −9.848 | 57.722 | 1.00 | 14.45 | Z | C |
| ATOM | 12321 | CA | PRO | Z | 119 | −27.872 | −11.639 | 59.145 | 1.00 | 21.24 | Z | C |
| ATOM | 12322 | CB | PRO | Z | 119 | −27.533 | −10.265 | 59.723 | 1.00 | 20.80 | Z | C |
| ATOM | 12323 | CG | PRO | Z | 119 | −28.643 | −9.404 | 59.162 | 1.00 | 21.93 | Z | C |
| ATOM | 12324 | C | PRO | Z | 119 | −28.838 | −12.338 | 60.077 | 1.00 | 23.61 | Z | C |
| ATOM | 12325 | O | PRO | Z | 119 | −30.042 | −12.298 | 59.868 | 1.00 | 29.56 | Z | O |
| ATOM | 12326 | N | SER | Z | 120 | −28.286 | −13.034 | 61.067 | 1.00 | 23.77 | Z | N |
| ATOM | 12327 | CA | SER | Z | 120 | −29.071 | −13.660 | 62.119 | 1.00 | 14.05 | Z | C |
| ATOM | 12328 | CB | SER | Z | 120 | −28.543 | −15.039 | 62.496 | 1.00 | 14.28 | Z | C |
| ATOM | 12329 | OG | SER | Z | 120 | −28.688 | −15.980 | 61.444 | 1.00 | 26.87 | Z | O |
| ATOM | 12330 | C | SER | Z | 120 | −28.747 | −12.684 | 63.252 | 1.00 | 18.83 | Z | C |
| ATOM | 12331 | O | SER | Z | 120 | −27.583 | −12.377 | 63.493 | 1.00 | 19.93 | Z | O |
| ATOM | 12332 | N | VAL | Z | 121 | −29.767 | −12.127 | 63.884 | 1.00 | 20.74 | Z | N |
| ATOM | 12333 | CA | VAL | Z | 121 | −29.564 | −11.173 | 64.955 | 1.00 | 21.26 | Z | C |
| ATOM | 12334 | CB | VAL | Z | 121 | −30.475 | −9.949 | 64.775 | 1.00 | 21.86 | Z | C |
| ATOM | 12335 | CG1 | VAL | Z | 121 | −30.253 | −8.945 | 65.900 | 1.00 | 22.34 | Z | C |
| ATOM | 12336 | CG2 | VAL | Z | 121 | −30.211 | −9.301 | 63.424 | 1.00 | 17.71 | Z | C |
| ATOM | 12337 | C | VAL | Z | 121 | −29.804 | −11.819 | 66.319 | 1.00 | 24.90 | Z | C |
| ATOM | 12338 | O | VAL | Z | 121 | −30.866 | −12.382 | 66.573 | 1.00 | 28.09 | Z | O |
| ATOM | 12339 | N | TYR | Z | 122 | −28.783 | −11.785 | 67.169 | 1.00 | 19.59 | Z | N |
| ATOM | 12340 | CA | TYR | Z | 122 | −28.874 | −12.359 | 68.506 | 1.00 | 23.41 | Z | C |
| ATOM | 12341 | CB | TYR | Z | 122 | −27.786 | −13.376 | 68.723 | 1.00 | 16.73 | Z | C |
| ATOM | 12342 | CG | TYR | Z | 122 | −27.843 | −14.464 | 67.710 | 1.00 | 22.97 | Z | C |
| ATOM | 12343 | CD1 | TYR | Z | 122 | −28.859 | −15.407 | 67.742 | 1.00 | 21.11 | Z | C |
| ATOM | 12344 | CE1 | TYR | Z | 122 | −28.902 | −16.436 | 66.821 | 1.00 | 21.68 | Z | C |
| ATOM | 12345 | CD2 | TYR | Z | 122 | −26.864 | −14.573 | 66.724 | 1.00 | 21.53 | Z | C |
| ATOM | 12346 | CE2 | TYR | Z | 122 | −26.890 | −15.596 | 65.810 | 1.00 | 25.16 | Z | C |
| ATOM | 12347 | CZ | TYR | Z | 122 | −27.912 | −16.532 | 65.863 | 1.00 | 26.17 | Z | C |
| ATOM | 12348 | OH | TYR | Z | 122 | −27.909 | −17.586 | 64.987 | 1.00 | 29.07 | Z | O |
| ATOM | 12349 | C | TYR | Z | 122 | −28.787 | −11.277 | 69.568 | 1.00 | 21.68 | Z | C |
| ATOM | 12350 | O | TYR | Z | 122 | −28.035 | −10.320 | 69.434 | 1.00 | 30.61 | Z | O |
| ATOM | 12351 | N | PRO | Z | 123 | −29.568 | −11.420 | 70.638 | 1.00 | 21.82 | Z | N |
| ATOM | 12352 | CD | PRO | Z | 123 | −30.413 | −12.595 | 70.936 | 1.00 | 17.66 | Z | C |
| ATOM | 12353 | CA | PRO | Z | 123 | −29.604 | −10.453 | 71.735 | 1.00 | 20.29 | Z | C |
| ATOM | 12354 | CB | PRO | Z | 123 | −30.874 | −10.852 | 72.468 | 1.00 | 16.75 | Z | C |
| ATOM | 12355 | CG | PRO | Z | 123 | −30.826 | −12.354 | 72.357 | 1.00 | 15.16 | Z | C |
| ATOM | 12356 | C | PRO | Z | 123 | −28.389 | −10.564 | 72.639 | 1.00 | 21.98 | Z | C |
| ATOM | 12357 | O | PRO | Z | 123 | −27.874 | −11.660 | 72.870 | 1.00 | 27.13 | Z | O |
| ATOM | 12358 | N | LEU | Z | 124 | −27.876 | −9.416 | 73.056 | 1.00 | 21.90 | Z | N |
| ATOM | 12359 | CA | LEU | Z | 124 | −26.742 | −9.362 | 73.970 | 1.00 | 23.19 | Z | C |
| ATOM | 12360 | CB | LEU | Z | 124 | −25.578 | −8.546 | 73.394 | 1.00 | 23.49 | Z | C |
| ATOM | 12361 | CG | LEU | Z | 124 | −24.927 | −9.053 | 72.105 | 1.00 | 20.50 | Z | C |
| ATOM | 12362 | CD1 | LEU | Z | 124 | −23.939 | −8.015 | 71.670 | 1.00 | 15.64 | Z | C |
| ATOM | 12363 | CD2 | LEU | Z | 124 | −24.246 | −10.422 | 72.290 | 1.00 | 17.07 | Z | C |
| ATOM | 12364 | C | LEU | Z | 124 | −27.289 | −8.717 | 75.229 | 1.00 | 24.40 | Z | C |
| ATOM | 12365 | O | LEU | Z | 124 | −27.558 | −7.495 | 75.293 | 1.00 | 19.50 | Z | O |
| ATOM | 12366 | N | ALA | Z | 125 | −27.562 | −9.587 | 76.187 | 1.00 | 26.50 | Z | N |
| ATOM | 12367 | CA | ALA | Z | 125 | −28.108 | −9.191 | 77.469 | 1.00 | 33.03 | Z | C |
| ATOM | 12368 | CB | ALA | Z | 125 | −29.446 | −9.903 | 77.729 | 1.00 | 26.69 | Z | C |
| ATOM | 12369 | C | ALA | Z | 125 | −27.114 | −9.569 | 78.537 | 1.00 | 33.25 | Z | C |
| ATOM | 12370 | O | ALA | Z | 125 | −26.429 | −10.587 | 78.434 | 1.00 | 32.83 | Z | O |
| ATOM | 12371 | N | PRO | Z | 126 | −27.055 | −8.773 | 79.607 | 1.00 | 40.77 | Z | N |
| ATOM | 12372 | CD | PRO | Z | 126 | −27.795 | −7.502 | 79.767 | 1.00 | 36.42 | Z | C |
| ATOM | 12373 | CA | PRO | Z | 126 | −26.142 | −9.004 | 80.730 | 1.00 | 45.96 | Z | C |
| ATOM | 12374 | CB | PRO | Z | 126 | −26.010 | −7.611 | 81.323 | 1.00 | 45.99 | Z | C |
| ATOM | 12375 | CG | PRO | Z | 126 | −27.432 | −7.072 | 81.167 | 1.00 | 40.73 | Z | C |
| ATOM | 12376 | C | PRO | Z | 126 | −26.756 | −9.914 | 81.768 | 1.00 | 55.00 | Z | C |
| ATOM | 12377 | O | PRO | Z | 126 | −27.978 | −9.954 | 81.908 | 1.00 | 59.40 | Z | O |
| ATOM | 12378 | N | VAL | Z | 127 | −25.913 | −10.637 | 82.497 | 1.00 | 64.18 | Z | N |
| ATOM | 12379 | CA | VAL | Z | 127 | −26.389 | −11.491 | 83.590 | 1.00 | 71.12 | Z | C |
| ATOM | 12380 | CB | VAL | Z | 127 | −27.192 | −12.758 | 83.113 | 1.00 | 71.67 | Z | C |
| ATOM | 12381 | CG1 | VAL | Z | 127 | −26.289 | −13.748 | 82.373 | 1.00 | 72.63 | Z | C |
| ATOM | 12382 | CG2 | VAL | Z | 127 | −27.901 | −13.424 | 84.316 | 1.00 | 67.37 | Z | C |
| ATOM | 12383 | C | VAL | Z | 127 | −25.248 | −11.857 | 84.532 | 1.00 | 73.65 | Z | C |
| ATOM | 12384 | O | VAL | Z | 127 | −25.061 | −13.020 | 84.909 | 1.00 | 76.99 | Z | O |
| ATOM | 12385 | N | CYS | Z | 128 | −24.469 | −10.844 | 84.898 | 1.00 | 76.41 | Z | N |
| ATOM | 12386 | CA | CYS | Z | 128 | −23.362 | −11.049 | 85.820 | 1.00 | 82.03 | Z | C |
| ATOM | 12387 | CB | CYS | Z | 128 | −22.315 | −9.946 | 85.657 | 1.00 | 81.60 | Z | C |
| ATOM | 12388 | SG | CYS | Z | 128 | −20.637 | −10.590 | 85.512 | 1.00 | 90.09 | Z | S |
| ATOM | 12389 | C | CYS | Z | 128 | −23.935 | −11.065 | 87.245 | 1.00 | 83.25 | Z | C |
| ATOM | 12390 | O | CYS | Z | 128 | −23.276 | −11.493 | 88.199 | 1.00 | 86.18 | Z | O |
| ATOM | 12391 | N | GLY | Z | 129 | −25.189 | −10.631 | 87.360 | 1.00 | 84.71 | Z | N |
| ATOM | 12392 | CA | GLY | Z | 129 | −25.870 | −10.601 | 88.640 | 1.00 | 82.28 | Z | C |
| ATOM | 12393 | C | GLY | Z | 129 | −26.959 | −9.545 | 88.660 | 1.00 | 83.70 | Z | C |
| ATOM | 12394 | O | GLY | Z | 129 | −27.964 | −9.652 | 87.946 | 1.00 | 79.98 | Z | O |
| ATOM | 12395 | N | ASP | Z | 130 | −26.736 | −8.509 | 89.467 | 1.00 | 83.98 | Z | N |
| ATOM | 12396 | CA | ASP | Z | 130 | −27.677 | −7.404 | 89.620 | 1.00 | 82.91 | Z | C |
| ATOM | 12397 | CB | ASP | Z | 130 | −27.973 | −7.165 | 91.116 | 1.00 | 83.81 | Z | C |
| ATOM | 12398 | CG | ASP | Z | 130 | −26.909 | −6.288 | 91.807 | 1.00 | 87.22 | Z | C |
| ATOM | 12399 | OD1 | ASP | Z | 130 | −25.702 | −6.634 | 91.765 | 1.00 | 89.02 | Z | O |
| ATOM | 12400 | OD2 | ASP | Z | 130 | −27.284 | −5.233 | 92.373 | 1.00 | 85.63 | Z | O |
| ATOM | 12401 | C | ASP | Z | 130 | −27.127 | −6.111 | 89.008 | 1.00 | 83.35 | Z | C |
| ATOM | 12402 | O | ASP | Z | 130 | −26.146 | −6.117 | 88.243 | 1.00 | 82.75 | Z | O |
| ATOM | 12403 | N | THR | Z | 131 | −27.776 | −5.010 | 89.383 | 1.00 | 81.54 | Z | N |
| ATOM | 12404 | CA | THR | Z | 131 | −27.409 | −3.664 | 88.962 | 1.00 | 80.60 | Z | C |
| ATOM | 12405 | CB | THR | Z | 131 | −28.603 | −2.677 | 89.184 | 1.00 | 81.35 | Z | C |
| ATOM | 12406 | OG1 | THR | Z | 131 | −28.106 | −1.368 | 89.497 | 1.00 | 79.36 | Z | O |
| ATOM | 12407 | CG2 | THR | Z | 131 | −29.529 | −3.163 | 90.311 | 1.00 | 77.40 | Z | C |
| ATOM | 12408 | C | THR | Z | 131 | −26.181 | −3.186 | 89.753 | 1.00 | 79.58 | Z | C |
| ATOM | 12409 | O | THR | Z | 131 | −26.273 | −2.930 | 90.959 | 1.00 | 75.70 | Z | O |
| ATOM | 12410 | N | THR | Z | 132 | −25.033 | −3.108 | 89.076 | 1.00 | 80.07 | Z | N |
| ATOM | 12411 | CA | THR | Z | 132 | −23.780 | −2.652 | 89.694 | 1.00 | 79.15 | Z | C |
| ATOM | 12412 | CB | THR | Z | 132 | −22.577 | −3.543 | 89.215 | 1.00 | 79.96 | Z | C |
| ATOM | 12413 | OG1 | THR | Z | 132 | −22.907 | −4.931 | 89.405 | 1.00 | 78.08 | Z | O |
| ATOM | 12414 | CG2 | THR | Z | 132 | −21.294 | −3.232 | 90.004 | 1.00 | 75.82 | Z | C |
| ATOM | 12415 | C | THR | Z | 132 | −23.576 | −1.150 | 89.374 | 1.00 | 78.29 | Z | C |
| ATOM | 12416 | O | THR | Z | 132 | −22.506 | −0.572 | 89.603 | 1.00 | 83.61 | Z | O |
| ATOM | 12417 | N | GLY | Z | 133 | −24.648 | −0.530 | 88.885 | 1.00 | 74.67 | Z | N |
| ATOM | 12418 | CA | GLY | Z | 133 | −24.655 | 0.879 | 88.536 | 1.00 | 68.79 | Z | C |
| ATOM | 12419 | C | GLY | Z | 133 | −26.075 | 1.253 | 88.140 | 1.00 | 66.02 | Z | C |
| ATOM | 12420 | O | GLY | Z | 133 | −26.941 | 0.381 | 88.041 | 1.00 | 63.94 | Z | O |
| ATOM | 12421 | N | SER | Z | 134 | −26.339 | 2.544 | 87.963 | 1.00 | 62.02 | Z | N |
| ATOM | 12422 | CA | SER | Z | 134 | −27.671 | 2.986 | 87.557 | 1.00 | 56.89 | Z | C |
| ATOM | 12423 | CB | SER | Z | 134 | −27.907 | 4.453 | 87.972 | 1.00 | 58.46 | Z | C |
| ATOM | 12424 | OG | SER | Z | 134 | −26.924 | 5.338 | 87.442 | 1.00 | 53.87 | Z | O |
| ATOM | 12425 | C | SER | Z | 134 | −27.831 | 2.815 | 86.035 | 1.00 | 56.93 | Z | C |
| ATOM | 12426 | O | SER | Z | 134 | −28.909 | 3.028 | 85.483 | 1.00 | 55.90 | Z | O |
| ATOM | 12427 | N | SER | Z | 135 | −26.747 | 2.403 | 85.377 | 1.00 | 50.45 | Z | N |
| ATOM | 12428 | CA | SER | Z | 135 | −26.724 | 2.206 | 83.932 | 1.00 | 44.93 | Z | C |
| ATOM | 12429 | CB | SER | Z | 135 | −25.527 | 2.966 | 83.325 | 1.00 | 47.54 | Z | C |
| ATOM | 12430 | OG | SER | Z | 135 | −25.538 | 4.356 | 83.638 | 1.00 | 46.34 | Z | O |
| ATOM | 12431 | C | SER | Z | 135 | −26.644 | 0.711 | 83.554 | 1.00 | 40.66 | Z | C |
| ATOM | 12432 | O | SER | Z | 135 | −26.096 | −0.106 | 84.295 | 1.00 | 38.31 | Z | O |
| ATOM | 12433 | N | VAL | Z | 136 | −27.235 | 0.352 | 82.419 | 1.00 | 33.60 | Z | N |
| ATOM | 12434 | CA | VAL | Z | 136 | −27.204 | −1.028 | 81.941 | 1.00 | 32.00 | Z | C |
| ATOM | 12435 | CB | VAL | Z | 136 | −28.550 | −1.795 | 82.226 | 1.00 | 34.45 | Z | C |
| ATOM | 12436 | CG1 | VAL | Z | 136 | −29.694 | −1.222 | 81.430 | 1.00 | 39.66 | Z | C |
| ATOM | 12437 | CG2 | VAL | Z | 136 | −28.406 | −3.266 | 81.906 | 1.00 | 38.40 | Z | C |
| ATOM | 12438 | C | VAL | Z | 136 | −26.897 | −0.980 | 80.450 | 1.00 | 26.34 | Z | C |
| ATOM | 12439 | O | VAL | Z | 136 | −27.422 | −0.132 | 79.730 | 1.00 | 25.71 | Z | O |
| ATOM | 12440 | N | THR | Z | 137 | −25.982 | −1.840 | 80.011 | 1.00 | 28.33 | Z | N |
| ATOM | 12441 | CA | THR | Z | 137 | −25.572 | −1.894 | 78.606 | 1.00 | 27.97 | Z | C |
| ATOM | 12442 | CB | THR | Z | 137 | −24.020 | −1.825 | 78.494 | 1.00 | 26.53 | Z | C |
| ATOM | 12443 | OG1 | THR | Z | 137 | −23.558 | −0.618 | 79.107 | 1.00 | 30.17 | Z | O |
| ATOM | 12444 | CG2 | THR | Z | 137 | −23.580 | −1.807 | 77.052 | 1.00 | 29.75 | Z | C |
| ATOM | 12445 | C | THR | Z | 137 | −26.126 | −3.143 | 77.910 | 1.00 | 23.95 | Z | C |
| ATOM | 12446 | O | THR | Z | 137 | −25.851 | −4.268 | 78.312 | 1.00 | 30.90 | Z | O |
| ATOM | 12447 | N | LEU | Z | 138 | −26.911 | −2.923 | 76.867 | 1.00 | 22.54 | Z | N |
| ATOM | 12448 | CA | LEU | Z | 138 | −27.544 | −3.998 | 76.106 | 1.00 | 21.98 | Z | C |
| ATOM | 12449 | CB | LEU | Z | 138 | −29.055 | −3.734 | 76.009 | 1.00 | 19.05 | Z | C |
| ATOM | 12450 | CG | LEU | Z | 138 | −30.054 | −4.008 | 77.146 | 1.00 | 21.10 | Z | C |
| ATOM | 12451 | CD1 | LEU | Z | 138 | −29.428 | −4.784 | 78.277 | 1.00 | 13.93 | Z | C |
| ATOM | 12452 | CD2 | LEU | Z | 138 | −30.661 | −2.723 | 77.627 | 1.00 | 19.67 | Z | C |
| ATOM | 12453 | C | LEU | Z | 138 | −26.986 | −3.957 | 74.705 | 1.00 | 22.60 | Z | C |
| ATOM | 12454 | O | LEU | Z | 138 | −26.476 | −2.923 | 74.281 | 1.00 | 24.41 | Z | O |
| ATOM | 12455 | N | GLY | Z | 139 | −27.129 | −5.050 | 73.959 | 1.00 | 26.77 | Z | N |
| ATOM | 12456 | CA | GLY | Z | 139 | −26.647 | −5.037 | 72.588 | 1.00 | 27.08 | Z | C |
| ATOM | 12457 | C | GLY | Z | 139 | −27.198 | −6.117 | 71.686 | 1.00 | 26.52 | Z | C |
| ATOM | 12458 | O | GLY | Z | 139 | −27.995 | −6.945 | 72.114 | 1.00 | 29.38 | Z | O |
| ATOM | 12459 | N | CYS | Z | 140 | −26.840 | −6.071 | 70.410 | 0.50 | 25.62 | Z | N |
| ATOM | 12460 | CA | CYS | Z | 140 | −27.247 | −7.128 | 69.504 | 0.50 | 21.91 | Z | C |
| ATOM | 12461 | C | CYS | Z | 140 | −26.131 | −7.516 | 68.565 | 0.50 | 23.40 | Z | C |
| ATOM | 12462 | O | CYS | Z | 140 | −25.366 | −6.680 | 68.072 | 0.50 | 24.58 | Z | O |
| ATOM | 12463 | CB | CYS | Z | 140 | −28.573 | −6.867 | 68.779 | 0.50 | 24.48 | Z | C |
| ATOM | 12464 | SG | CYS | Z | 140 | −28.772 | −5.452 | 67.661 | 0.50 | 23.53 | Z | S |
| ATOM | 12465 | N | LEU | Z | 141 | −26.002 | −8.822 | 68.410 | 1.00 | 17.40 | Z | N |
| ATOM | 12466 | CA | LEU | Z | 141 | −24.986 | −9.421 | 67.598 | 1.00 | 18.88 | Z | C |
| ATOM | 12467 | CB | LEU | Z | 141 | −24.535 | −10.719 | 68.271 | 1.00 | 19.64 | Z | C |
| ATOM | 12468 | CG | LEU | Z | 141 | −23.415 | −11.508 | 67.606 | 1.00 | 20.82 | Z | C |
| ATOM | 12469 | CD1 | LEU | Z | 141 | −22.188 | −10.647 | 67.480 | 1.00 | 23.49 | Z | C |
| ATOM | 12470 | CD2 | LEU | Z | 141 | −23.110 | −12.720 | 68.439 | 1.00 | 19.54 | Z | C |
| ATOM | 12471 | C | LEU | Z | 141 | −25.596 | −9.675 | 66.202 | 1.00 | 22.20 | Z | C |
| ATOM | 12472 | O | LEU | Z | 141 | −26.479 | −10.514 | 66.036 | 1.00 | 21.39 | Z | O |
| ATOM | 12473 | N | VAL | Z | 142 | −25.205 | −8.854 | 65.235 | 1.00 | 18.35 | Z | N |
| ATOM | 12474 | CA | VAL | Z | 142 | −25.675 | −8.982 | 63.863 | 1.00 | 17.66 | Z | C |
| ATOM | 12475 | CB | VAL | Z | 142 | −25.720 | −7.611 | 63.204 | 1.00 | 17.42 | Z | C |
| ATOM | 12476 | CG1 | VAL | Z | 142 | −26.320 | −7.703 | 61.833 | 1.00 | 18.20 | Z | C |
| ATOM | 12477 | CG2 | VAL | Z | 142 | −26.518 | −6.649 | 64.080 | 1.00 | 15.33 | Z | C |
| ATOM | 12478 | C | VAL | Z | 142 | −24.659 | −9.901 | 63.192 | 1.00 | 15.08 | Z | C |
| ATOM | 12479 | O | VAL | Z | 142 | −23.612 | −9.458 | 62.729 | 1.00 | 19.77 | Z | O |
| ATOM | 12480 | N | LYS | Z | 143 | −24.991 | −11.186 | 63.147 | 1.00 | 15.84 | Z | N |
| ATOM | 12481 | CA | LYS | Z | 143 | −24.093 | −12.208 | 62.629 | 1.00 | 21.03 | Z | C |
| ATOM | 12482 | CB | LYS | Z | 143 | −23.915 | −13.263 | 63.725 | 1.00 | 17.63 | Z | C |
| ATOM | 12483 | CG | LYS | Z | 143 | −23.052 | −14.423 | 63.341 | 1.00 | 17.01 | Z | C |
| ATOM | 12484 | CD | LYS | Z | 143 | −23.012 | −15.440 | 64.446 | 1.00 | 16.71 | Z | C |
| ATOM | 12485 | CE | LYS | Z | 143 | −22.068 | −16.547 | 64.085 | 1.00 | 19.64 | Z | C |
| ATOM | 12486 | NZ | LYS | Z | 143 | −21.981 | −17.535 | 65.176 | 1.00 | 25.71 | Z | N |
| ATOM | 12487 | C | LYS | Z | 143 | −24.355 | −12.916 | 61.284 | 1.00 | 24.13 | Z | C |
| ATOM | 12488 | O | LYS | Z | 143 | −25.478 | −13.335 | 60.973 | 1.00 | 29.55 | Z | O |
| ATOM | 12489 | N | GLY | Z | 144 | −23.269 | −13.075 | 60.531 | 1.00 | 23.85 | Z | N |
| ATOM | 12490 | CA | GLY | Z | 144 | −23.275 | −13.789 | 59.263 | 1.00 | 23.07 | Z | C |
| ATOM | 12491 | C | GLY | Z | 144 | −24.014 | −13.217 | 58.081 | 1.00 | 24.59 | Z | C |
| ATOM | 12492 | O | GLY | Z | 144 | −24.845 | −13.901 | 57.491 | 1.00 | 22.42 | Z | O |
| ATOM | 12493 | N | TYR | Z | 145 | −23.683 | −11.991 | 57.706 | 1.00 | 18.90 | Z | N |
| ATOM | 12494 | CA | TYR | Z | 145 | −24.329 | −11.368 | 56.577 | 1.00 | 22.32 | Z | C |
| ATOM | 12495 | CB | TYR | Z | 145 | −25.062 | −10.107 | 57.018 | 1.00 | 24.48 | Z | C |
| ATOM | 12496 | CG | TYR | Z | 145 | −24.156 | −9.036 | 57.566 | 1.00 | 27.59 | Z | C |
| ATOM | 12497 | CD1 | TYR | Z | 145 | −23.792 | −9.024 | 58.912 | 1.00 | 24.55 | Z | C |
| ATOM | 12498 | CE1 | TYR | Z | 145 | −23.000 | −8.024 | 59.422 | 1.00 | 19.97 | Z | C |
| ATOM | 12499 | CD2 | TYR | Z | 145 | −23.690 | −8.011 | 56.748 | 1.00 | 24.75 | Z | C |
| ATOM | 12500 | CE2 | TYR | Z | 145 | −22.897 | −7.011 | 57.249 | 1.00 | 23.50 | Z | C |
| ATOM | 12501 | CZ | TYR | Z | 145 | −22.554 | −7.020 | 58.588 | 1.00 | 23.85 | Z | C |
| ATOM | 12502 | OH | TYR | Z | 145 | −21.762 | −6.016 | 59.083 | 1.00 | 20.28 | Z | O |
| ATOM | 12503 | C | TYR | Z | 145 | −23.362 | −11.048 | 55.449 | 1.00 | 20.55 | Z | C |
| ATOM | 12504 | O | TYR | Z | 145 | −22.152 | −11.137 | 55.601 | 1.00 | 18.58 | Z | O |
| ATOM | 12505 | N | PHE | Z | 146 | −23.924 | −10.694 | 54.302 | 1.00 | 22.72 | Z | N |
| ATOM | 12506 | CA | PHE | Z | 146 | −23.140 | −10.358 | 53.124 | 1.00 | 24.20 | Z | C |
| ATOM | 12507 | CB | PHE | Z | 146 | −22.385 | −11.582 | 52.614 | 1.00 | 21.88 | Z | C |
| ATOM | 12508 | CG | PHE | Z | 146 | −21.406 | −11.268 | 51.523 | 1.00 | 28.03 | Z | C |
| ATOM | 12509 | CD1 | PHE | Z | 146 | −21.831 | −11.141 | 50.199 | 1.00 | 25.93 | Z | C |
| ATOM | 12510 | CD2 | PHE | Z | 146 | −20.068 | −11.064 | 51.818 | 1.00 | 20.25 | Z | C |
| ATOM | 12511 | CE1 | PHE | Z | 146 | −20.936 | −10.811 | 49.191 | 1.00 | 28.09 | Z | C |
| ATOM | 12512 | CE2 | PHE | Z | 146 | −19.165 | −10.733 | 50.813 | 1.00 | 27.46 | Z | C |
| ATOM | 12513 | CZ | PHE | Z | 146 | −19.598 | −10.607 | 49.498 | 1.00 | 27.35 | Z | C |
| ATOM | 12514 | C | PHE | Z | 146 | −24.055 | −9.820 | 52.028 | 1.00 | 23.04 | Z | C |
| ATOM | 12515 | O | PHE | Z | 146 | −25.118 | −10.349 | 51.782 | 1.00 | 29.73 | Z | O |
| ATOM | 12516 | N | PRO | Z | 147 | −23.649 | −8.744 | 51.365 | 1.00 | 24.46 | Z | N |
| ATOM | 12517 | CD | PRO | Z | 147 | −24.316 | −8.185 | 50.177 | 1.00 | 24.09 | Z | C |
| ATOM | 12518 | CA | PRO | Z | 147 | −22.394 | −8.047 | 51.618 | 1.00 | 26.14 | Z | C |
| ATOM | 12519 | CB | PRO | Z | 147 | −22.089 | −7.454 | 50.250 | 1.00 | 23.72 | Z | C |
| ATOM | 12520 | CG | PRO | Z | 147 | −23.431 | −6.982 | 49.828 | 1.00 | 20.98 | Z | C |
| ATOM | 12521 | C | PRO | Z | 147 | −22.648 | −6.932 | 52.630 | 1.00 | 26.90 | Z | C |
| ATOM | 12522 | O | PRO | Z | 147 | −23.739 | −6.809 | 53.189 | 1.00 | 26.99 | Z | O |
| ATOM | 12523 | N | GLU | Z | 148 | −21.629 | −6.125 | 52.860 | 1.00 | 28.82 | Z | N |
| ATOM | 12524 | CA | GLU | Z | 148 | −21.772 | −5.007 | 53.757 | 1.00 | 25.31 | Z | C |
| ATOM | 12525 | CB | GLU | Z | 148 | −20.408 | −4.415 | 54.040 | 1.00 | 25.55 | Z | C |
| ATOM | 12526 | CG | GLU | Z | 148 | −19.549 | −5.301 | 54.883 | 1.00 | 31.47 | Z | C |
| ATOM | 12527 | CD | GLU | Z | 148 | −18.363 | −4.558 | 55.405 | 1.00 | 36.98 | Z | C |
| ATOM | 12528 | OE1 | GLU | Z | 148 | −18.564 | −3.747 | 56.346 | 1.00 | 32.05 | Z | O |
| ATOM | 12529 | OE2 | GLU | Z | 148 | −17.258 | −4.764 | 54.843 | 1.00 | 32.73 | Z | O |
| ATOM | 12530 | C | GLU | Z | 148 | −22.629 | −4.015 | 52.983 | 1.00 | 22.81 | Z | C |
| ATOM | 12531 | O | GLU | Z | 148 | −22.627 | −4.027 | 51.757 | 1.00 | 35.03 | Z | O |
| ATOM | 12532 | N | PRO | Z | 149 | −23.343 | −3.127 | 53.682 | 1.00 | 19.35 | Z | N |
| ATOM | 12533 | CD | PRO | Z | 149 | −23.928 | −1.921 | 53.060 | 1.00 | 14.42 | Z | C |
| ATOM | 12534 | CA | PRO | Z | 149 | −23.368 | −3.024 | 55.151 | 1.00 | 22.05 | Z | C |
| ATOM | 12535 | CB | PRO | Z | 149 | −23.091 | −1.544 | 55.367 | 1.00 | 15.18 | Z | C |
| ATOM | 12536 | CG | PRO | Z | 149 | −23.959 | −0.896 | 54.246 | 1.00 | 20.28 | Z | C |
| ATOM | 12537 | C | PRO | Z | 149 | −24.737 | −3.393 | 55.744 | 1.00 | 23.36 | Z | C |
| ATOM | 12538 | O | PRO | Z | 149 | −25.561 | −4.020 | 55.090 | 1.00 | 31.81 | Z | O |
| ATOM | 12539 | N | VAL | Z | 150 | −24.940 | −3.033 | 57.004 | 1.00 | 27.93 | Z | N |
| ATOM | 12540 | CA | VAL | Z | 150 | −26.205 | −3.226 | 57.708 | 1.00 | 30.96 | Z | C |
| ATOM | 12541 | CB | VAL | Z | 150 | −26.229 | −4.436 | 58.688 | 1.00 | 27.64 | Z | C |
| ATOM | 12542 | CG1 | VAL | Z | 150 | −26.283 | −5.752 | 57.938 | 1.00 | 27.09 | Z | C |
| ATOM | 12543 | CG2 | VAL | Z | 150 | −25.033 | −4.387 | 59.626 | 1.00 | 28.21 | Z | C |
| ATOM | 12544 | C | VAL | Z | 150 | −26.317 | −1.981 | 58.565 | 1.00 | 34.41 | Z | C |
| ATOM | 12545 | O | VAL | Z | 150 | −25.302 | −1.395 | 58.933 | 1.00 | 39.14 | Z | O |
| ATOM | 12546 | N | THR | Z | 151 | −27.538 | −1.518 | 58.803 | 1.00 | 37.00 | Z | N |
| ATOM | 12547 | CA | THR | Z | 151 | −27.734 | −0.363 | 59.669 | 1.00 | 31.14 | Z | C |
| ATOM | 12548 | CB | THR | Z | 151 | −28.713 | 0.673 | 59.106 | 1.00 | 30.92 | Z | C |
| ATOM | 12549 | OG1 | THR | Z | 151 | −29.942 | 0.029 | 58.771 | 1.00 | 38.47 | Z | O |
| ATOM | 12550 | CG2 | THR | Z | 151 | −28.129 | 1.356 | 57.876 | 1.00 | 37.24 | Z | C |
| ATOM | 12551 | C | THR | Z | 151 | −28.306 | −0.929 | 60.951 | 1.00 | 33.48 | Z | C |
| ATOM | 12552 | O | THR | Z | 151 | −28.784 | −2.070 | 60.980 | 1.00 | 32.31 | Z | O |
| ATOM | 12553 | N | LEU | Z | 152 | −28.291 | −0.120 | 62.001 | 1.00 | 29.77 | Z | N |
| ATOM | 12554 | CA | LEU | Z | 152 | −28.767 | −0.562 | 63.291 | 1.00 | 27.09 | Z | C |
| ATOM | 12555 | CB | LEU | Z | 152 | −27.559 | −1.056 | 64.082 | 1.00 | 36.17 | Z | C |
| ATOM | 12556 | CG | LEU | Z | 152 | −27.665 | −2.094 | 65.183 | 1.00 | 30.48 | Z | C |
| ATOM | 12557 | CD1 | LEU | Z | 152 | −28.889 | −1.899 | 66.064 | 1.00 | 38.42 | Z | C |
| ATOM | 12558 | CD2 | LEU | Z | 152 | −27.669 | −3.424 | 64.493 | 1.00 | 42.81 | Z | C |
| ATOM | 12559 | C | LEU | Z | 152 | −29.333 | 0.625 | 64.026 | 1.00 | 27.72 | Z | C |
| ATOM | 12560 | O | LEU | Z | 152 | −28.687 | 1.668 | 64.076 | 1.00 | 27.98 | Z | O |
| ATOM | 12561 | N | THR | Z | 153 | −30.531 | 0.477 | 64.588 | 1.00 | 23.19 | Z | N |
| ATOM | 12562 | CA | THR | Z | 153 | −31.131 | 1.554 | 65.376 | 1.00 | 24.38 | Z | C |
| ATOM | 12563 | CB | THR | Z | 153 | −32.280 | 2.318 | 64.655 | 1.00 | 28.26 | Z | C |
| ATOM | 12564 | OG1 | THR | Z | 153 | −33.352 | 1.416 | 64.330 | 1.00 | 37.98 | Z | O |
| ATOM | 12565 | CG2 | THR | Z | 153 | −31.776 | 3.023 | 63.408 | 1.00 | 24.97 | Z | C |
| ATOM | 12566 | C | THR | Z | 153 | −31.678 | 0.942 | 66.647 | 1.00 | 22.19 | Z | C |
| ATOM | 12567 | O | THR | Z | 153 | −31.681 | −0.278 | 66.791 | 1.00 | 23.48 | Z | O |
| ATOM | 12568 | N | TRP | Z | 154 | −32.094 | 1.780 | 67.589 | 1.00 | 15.91 | Z | N |
| ATOM | 12569 | CA | TRP | Z | 154 | −32.655 | 1.265 | 68.821 | 1.00 | 12.44 | Z | C |
| ATOM | 12570 | CB | TRP | Z | 154 | −31.689 | 1.455 | 69.972 | 1.00 | 16.60 | Z | C |
| ATOM | 12571 | CG | TRP | Z | 154 | −30.525 | 0.569 | 69.848 | 1.00 | 13.05 | Z | C |
| ATOM | 12572 | CD2 | TRP | Z | 154 | −30.376 | −0.720 | 70.436 | 1.00 | 14.05 | Z | C |
| ATOM | 12573 | CE2 | TRP | Z | 154 | −29.097 | −1.201 | 70.075 | 1.00 | 12.56 | Z | C |
| ATOM | 12574 | CE3 | TRP | Z | 154 | −31.196 | −1.517 | 71.238 | 1.00 | 10.79 | Z | C |
| ATOM | 12575 | CD1 | TRP | Z | 154 | −29.376 | 0.817 | 69.163 | 1.00 | 14.56 | Z | C |
| ATOM | 12576 | NE1 | TRP | Z | 154 | −28.505 | −0.245 | 69.295 | 1.00 | 18.19 | Z | N |
| ATOM | 12577 | CZ2 | TRP | Z | 154 | −28.619 | −2.437 | 70.492 | 1.00 | 15.96 | Z | C |
| ATOM | 12578 | CZ3 | TRP | Z | 154 | −30.722 | −2.748 | 71.654 | 1.00 | 14.96 | Z | C |
| ATOM | 12579 | CH2 | TRP | Z | 154 | −29.440 | −3.197 | 71.280 | 1.00 | 19.18 | Z | C |
| ATOM | 12580 | C | TRP | Z | 154 | −33.965 | 1.938 | 69.083 | 1.00 | 15.22 | Z | C |
| ATOM | 12581 | O | TRP | Z | 154 | −34.065 | 3.160 | 69.021 | 1.00 | 19.35 | Z | O |
| ATOM | 12582 | N | ASN | Z | 155 | −34.972 | 1.117 | 69.374 | 1.00 | 22.53 | Z | N |
| ATOM | 12583 | CA | ASN | Z | 155 | −36.345 | 1.575 | 69.589 | 1.00 | 29.12 | Z | C |
| ATOM | 12584 | CB | ASN | Z | 155 | −36.543 | 2.180 | 70.997 | 1.00 | 23.96 | Z | C |
| ATOM | 12585 | CG | ASN | Z | 155 | −36.647 | 1.116 | 72.084 | 1.00 | 25.00 | Z | C |
| ATOM | 12586 | OD1 | ASN | Z | 155 | −36.531 | −0.090 | 71.832 | 1.00 | 30.24 | Z | O |
| ATOM | 12587 | ND2 | ASN | Z | 155 | −36.876 | 1.558 | 73.300 | 1.00 | 30.75 | Z | N |
| ATOM | 12588 | C | ASN | Z | 155 | −36.736 | 2.526 | 68.441 | 1.00 | 30.79 | Z | C |
| ATOM | 12589 | O | ASN | Z | 155 | −37.172 | 3.659 | 68.649 | 1.00 | 36.13 | Z | O |
| ATOM | 12590 | N | SER | Z | 156 | −36.462 | 2.056 | 67.222 | 1.00 | 35.24 | Z | N |
| ATOM | 12591 | CA | SER | Z | 156 | −36.767 | 2.759 | 65.979 | 1.00 | 37.99 | Z | C |
| ATOM | 12592 | CB | SER | Z | 156 | −38.279 | 2.834 | 65.825 | 1.00 | 37.18 | Z | C |
| ATOM | 12593 | OG | SER | Z | 156 | −38.834 | 1.568 | 66.164 | 1.00 | 35.40 | Z | O |
| ATOM | 12594 | C | SER | Z | 156 | −36.119 | 4.136 | 65.822 | 1.00 | 41.24 | Z | C |
| ATOM | 12595 | O | SER | Z | 156 | −36.466 | 4.900 | 64.909 | 1.00 | 45.34 | Z | O |
| ATOM | 12596 | N | GLY | Z | 157 | −35.135 | 4.417 | 66.674 | 1.00 | 40.13 | Z | N |
| ATOM | 12597 | CA | GLY | Z | 157 | −34.447 | 5.693 | 66.628 | 1.00 | 42.70 | Z | C |
| ATOM | 12598 | C | GLY | Z | 157 | −34.700 | 6.533 | 67.865 | 1.00 | 44.48 | Z | C |
| ATOM | 12599 | O | GLY | Z | 157 | −34.086 | 7.583 | 68.041 | 1.00 | 50.13 | Z | O |
| ATOM | 12600 | N | SER | Z | 158 | −35.621 | 6.086 | 68.711 | 1.00 | 43.82 | Z | N |
| ATOM | 12601 | CA | SER | Z | 158 | −35.946 | 6.794 | 69.943 | 1.00 | 47.12 | Z | C |
| ATOM | 12602 | CB | SER | Z | 158 | −37.082 | 6.090 | 70.670 | 1.00 | 46.20 | Z | C |
| ATOM | 12603 | OG | SER | Z | 158 | −38.282 | 6.212 | 69.927 | 1.00 | 57.63 | Z | O |
| ATOM | 12604 | C | SER | Z | 158 | −34.731 | 6.877 | 70.845 | 1.00 | 46.55 | Z | C |
| ATOM | 12605 | O | SER | Z | 158 | −34.461 | 7.924 | 71.434 | 1.00 | 53.69 | Z | O |
| ATOM | 12606 | N | LEU | Z | 159 | −34.012 | 5.762 | 70.961 | 1.00 | 44.65 | Z | N |
| ATOM | 12607 | CA | LEU | Z | 159 | −32.800 | 5.703 | 71.769 | 1.00 | 40.54 | Z | C |
| ATOM | 12608 | CB | LEU | Z | 159 | −32.604 | 4.293 | 72.338 | 1.00 | 30.15 | Z | C |
| ATOM | 12609 | CG | LEU | Z | 159 | −33.599 | 3.738 | 73.374 | 1.00 | 37.57 | Z | C |
| ATOM | 12610 | CD1 | LEU | Z | 159 | −33.227 | 2.295 | 73.745 | 1.00 | 26.86 | Z | C |
| ATOM | 12611 | CD2 | LEU | Z | 159 | −33.624 | 4.610 | 74.643 | 1.00 | 29.46 | Z | C |
| ATOM | 12612 | C | LEU | Z | 159 | −31.647 | 6.050 | 70.834 | 1.00 | 43.64 | Z | C |
| ATOM | 12613 | O | LEU | Z | 159 | −31.219 | 5.211 | 70.045 | 1.00 | 49.74 | Z | O |
| ATOM | 12614 | N | SER | Z | 160 | −31.174 | 7.293 | 70.894 | 1.00 | 50.87 | Z | N |
| ATOM | 12615 | CA | SER | Z | 160 | −30.075 | 7.750 | 70.037 | 1.00 | 50.55 | Z | C |
| ATOM | 12616 | CB | SER | Z | 160 | −30.460 | 9.060 | 69.347 | 1.00 | 53.42 | Z | C |
| ATOM | 12617 | OG | SER | Z | 160 | −30.821 | 10.051 | 70.295 | 1.00 | 56.98 | Z | O |
| ATOM | 12618 | C | SER | Z | 160 | −28.761 | 7.934 | 70.782 | 1.00 | 47.65 | Z | C |
| ATOM | 12619 | O | SER | Z | 160 | −27.692 | 7.693 | 70.226 | 1.00 | 53.03 | Z | O |
| ATOM | 12620 | N | SER | Z | 161 | −28.841 | 8.403 | 72.023 | 1.00 | 46.48 | Z | N |
| ATOM | 12621 | CA | SER | Z | 161 | −27.646 | 8.617 | 72.832 | 1.00 | 44.47 | Z | C |
| ATOM | 12622 | CB | SER | Z | 161 | −27.896 | 9.683 | 73.889 | 1.00 | 48.39 | Z | C |
| ATOM | 12623 | OG | SER | Z | 161 | −29.001 | 9.322 | 74.695 | 1.00 | 57.63 | Z | O |
| ATOM | 12624 | C | SER | Z | 161 | −27.217 | 7.315 | 73.489 | 1.00 | 40.06 | Z | C |
| ATOM | 12625 | O | SER | Z | 161 | −28.038 | 6.447 | 73.781 | 1.00 | 42.49 | Z | O |
| ATOM | 12626 | N | GLY | Z | 162 | −25.923 | 7.173 | 73.710 | 1.00 | 36.43 | Z | N |
| ATOM | 12627 | CA | GLY | Z | 162 | −25.427 | 5.952 | 74.311 | 1.00 | 35.35 | Z | C |
| ATOM | 12628 | C | GLY | Z | 162 | −25.423 | 4.768 | 73.353 | 1.00 | 34.07 | Z | C |
| ATOM | 12629 | O | GLY | Z | 162 | −25.364 | 3.621 | 73.786 | 1.00 | 34.80 | Z | O |
| ATOM | 12630 | N | VAL | Z | 163 | −25.522 | 5.036 | 72.054 | 1.00 | 30.57 | Z | N |
| ATOM | 12631 | CA | VAL | Z | 163 | −25.505 | 3.973 | 71.044 | 1.00 | 28.33 | Z | C |
| ATOM | 12632 | CB | VAL | Z | 163 | −26.512 | 4.259 | 69.894 | 1.00 | 29.17 | Z | C |
| ATOM | 12633 | CG1 | VAL | Z | 163 | −26.348 | 3.244 | 68.784 | 1.00 | 25.37 | Z | C |
| ATOM | 12634 | CG2 | VAL | Z | 163 | −27.920 | 4.254 | 70.409 | 1.00 | 25.68 | Z | C |
| ATOM | 12635 | C | VAL | Z | 163 | −24.111 | 3.859 | 70.421 | 1.00 | 27.33 | Z | C |
| ATOM | 12636 | O | VAL | Z | 163 | −23.539 | 4.858 | 69.994 | 1.00 | 26.17 | Z | O |
| ATOM | 12637 | N | HIS | Z | 164 | −23.582 | 2.641 | 70.372 | 1.00 | 21.17 | Z | N |
| ATOM | 12638 | CA | HIS | Z | 164 | −22.276 | 2.386 | 69.779 | 1.00 | 25.97 | Z | C |
| ATOM | 12639 | CB | HIS | Z | 164 | −21.242 | 2.074 | 70.876 | 1.00 | 26.70 | Z | C |
| ATOM | 12640 | CG | HIS | Z | 164 | −20.911 | 3.244 | 71.755 | 1.00 | 32.82 | Z | C |
| ATOM | 12641 | CD2 | HIS | Z | 164 | −20.221 | 4.382 | 71.501 | 1.00 | 31.93 | Z | C |
| ATOM | 12642 | ND1 | HIS | Z | 164 | −21.288 | 3.313 | 73.081 | 1.00 | 32.81 | Z | N |
| ATOM | 12643 | CE1 | HIS | Z | 164 | −20.840 | 4.440 | 73.607 | 1.00 | 33.92 | Z | C |
| ATOM | 12644 | NE2 | HIS | Z | 164 | −20.189 | 5.106 | 72.669 | 1.00 | 32.46 | Z | N |
| ATOM | 12645 | C | HIS | Z | 164 | −22.363 | 1.229 | 68.768 | 1.00 | 26.01 | Z | C |
| ATOM | 12646 | O | HIS | Z | 164 | −22.709 | 0.099 | 69.121 | 1.00 | 26.55 | Z | O |
| ATOM | 12647 | N | THR | Z | 165 | −22.072 | 1.511 | 67.507 | 1.00 | 23.20 | Z | N |
| ATOM | 12648 | CA | THR | Z | 165 | −22.124 | 0.465 | 66.489 | 1.00 | 24.22 | Z | C |
| ATOM | 12649 | CB | THR | Z | 165 | −23.048 | 0.874 | 65.342 | 1.00 | 25.04 | Z | C |
| ATOM | 12650 | OG1 | THR | Z | 165 | −24.333 | 1.193 | 65.898 | 1.00 | 34.00 | Z | O |
| ATOM | 12651 | CG2 | THR | Z | 165 | −23.208 | −0.263 | 64.342 | 1.00 | 23.04 | Z | C |
| ATOM | 12652 | C | THR | Z | 165 | −20.711 | 0.245 | 66.017 | 1.00 | 19.63 | Z | C |
| ATOM | 12653 | O | THR | Z | 165 | −20.119 | 1.113 | 65.402 | 1.00 | 23.25 | Z | O |
| ATOM | 12654 | N | PHE | Z | 166 | −20.176 | −0.922 | 66.340 | 1.00 | 18.65 | Z | N |
| ATOM | 12655 | CA | PHE | Z | 166 | −18.810 | −1.278 | 66.033 | 1.00 | 18.90 | Z | C |
| ATOM | 12656 | CB | PHE | Z | 166 | −18.338 | −2.275 | 67.081 | 1.00 | 22.50 | Z | C |
| ATOM | 12657 | CG | PHE | Z | 166 | −18.573 | −1.821 | 68.494 | 1.00 | 23.99 | Z | C |
| ATOM | 12658 | CD1 | PHE | Z | 166 | −19.789 | −2.078 | 69.133 | 1.00 | 20.75 | Z | C |
| ATOM | 12659 | CD2 | PHE | Z | 166 | −17.573 | −1.153 | 69.200 | 1.00 | 25.50 | Z | C |
| ATOM | 12660 | CE1 | PHE | Z | 166 | −20.006 | −1.680 | 70.460 | 1.00 | 24.71 | Z | C |
| ATOM | 12661 | CE2 | PHE | Z | 166 | −17.776 | −0.749 | 70.533 | 1.00 | 24.56 | Z | C |
| ATOM | 12662 | CZ | PHE | Z | 166 | −18.995 | −1.015 | 71.163 | 1.00 | 25.09 | Z | C |
| ATOM | 12663 | C | PHE | Z | 166 | −18.545 | −1.787 | 64.614 | 1.00 | 26.67 | Z | C |
| ATOM | 12664 | O | PHE | Z | 166 | −19.475 | −2.100 | 63.864 | 1.00 | 30.92 | Z | O |
| ATOM | 12665 | N | PRO | Z | 167 | −17.268 | −1.785 | 64.194 | 1.00 | 27.33 | Z | N |
| ATOM | 12666 | CD | PRO | Z | 167 | −16.144 | −1.062 | 64.815 | 1.00 | 25.01 | Z | C |
| ATOM | 12667 | CA | PRO | Z | 167 | −16.902 | −2.252 | 62.852 | 1.00 | 26.15 | Z | C |
| ATOM | 12668 | CB | PRO | Z | 167 | −15.416 | −1.888 | 62.756 | 1.00 | 26.55 | Z | C |
| ATOM | 12669 | CG | PRO | Z | 167 | −15.312 | −0.675 | 63.606 | 1.00 | 28.24 | Z | C |
| ATOM | 12670 | C | PRO | Z | 167 | −17.112 | −3.744 | 62.664 | 1.00 | 25.02 | Z | C |
| ATOM | 12671 | O | PRO | Z | 167 | −17.114 | −4.526 | 63.613 | 1.00 | 30.15 | Z | O |
| ATOM | 12672 | N | ALA | Z | 168 | −17.291 | −4.135 | 61.423 | 1.00 | 24.39 | Z | N |
| ATOM | 12673 | CA | ALA | Z | 168 | −17.499 | −5.531 | 61.115 | 1.00 | 28.92 | Z | C |
| ATOM | 12674 | CB | ALA | Z | 168 | −18.263 | −5.638 | 59.809 | 1.00 | 29.44 | Z | C |
| ATOM | 12675 | C | ALA | Z | 168 | −16.191 | −6.306 | 61.021 | 1.00 | 25.96 | Z | C |
| ATOM | 12676 | O | ALA | Z | 168 | −15.153 | −5.741 | 60.700 | 1.00 | 29.18 | Z | O |
| ATOM | 12677 | N | VAL | Z | 169 | −16.244 | −7.578 | 61.392 | 1.00 | 25.85 | Z | N |
| ATOM | 12678 | CA | VAL | Z | 169 | −15.098 | −8.468 | 61.276 | 1.00 | 26.23 | Z | C |
| ATOM | 12679 | CB | VAL | Z | 169 | −14.655 | −9.081 | 62.607 | 1.00 | 24.56 | Z | C |
| ATOM | 12680 | CG1 | VAL | Z | 169 | −14.226 | −7.991 | 63.537 | 1.00 | 31.04 | Z | C |
| ATOM | 12681 | CG2 | VAL | Z | 169 | −15.758 | −9.930 | 63.216 | 1.00 | 26.41 | Z | C |
| ATOM | 12682 | C | VAL | Z | 169 | −15.563 | −9.570 | 60.339 | 1.00 | 30.09 | Z | C |
| ATOM | 12683 | O | VAL | Z | 169 | −16.766 | −9.802 | 60.195 | 1.00 | 28.99 | Z | O |
| ATOM | 12684 | N | LEU | Z | 170 | −14.614 | −10.249 | 59.710 | 1.00 | 34.70 | Z | N |
| ATOM | 12685 | CA | LEU | Z | 170 | −14.931 | −11.302 | 58.766 | 1.00 | 39.05 | Z | C |
| ATOM | 12686 | CB | LEU | Z | 170 | −14.107 | −11.082 | 57.487 | 1.00 | 37.28 | Z | C |
| ATOM | 12687 | CG | LEU | Z | 170 | −14.447 | −11.692 | 56.118 | 1.00 | 40.98 | Z | C |
| ATOM | 12688 | CD1 | LEU | Z | 170 | −15.842 | −11.316 | 55.662 | 1.00 | 27.04 | Z | C |
| ATOM | 12689 | CD2 | LEU | Z | 170 | −13.416 | −11.178 | 55.104 | 1.00 | 28.79 | Z | C |
| ATOM | 12690 | C | LEU | Z | 170 | −14.692 | −12.688 | 59.366 | 1.00 | 43.90 | Z | C |
| ATOM | 12691 | O | LEU | Z | 170 | −13.543 | −13.099 | 59.577 | 1.00 | 52.95 | Z | O |
| ATOM | 12692 | N | GLN | Z | 171 | −15.788 | −13.360 | 59.718 | 1.00 | 43.79 | Z | N |
| ATOM | 12693 | CA | GLN | Z | 171 | −15.736 | −14.709 | 60.272 | 1.00 | 45.95 | Z | C |
| ATOM | 12694 | CB | GLN | Z | 171 | −16.596 | −14.839 | 61.542 | 1.00 | 54.65 | Z | C |
| ATOM | 12695 | CG | GLN | Z | 171 | −16.044 | −14.132 | 62.796 | 1.00 | 62.10 | Z | C |
| ATOM | 12696 | CD | GLN | Z | 171 | −14.666 | −14.645 | 63.239 | 1.00 | 69.47 | Z | C |
| ATOM | 12697 | OE1 | GLN | Z | 171 | −14.457 | −15.856 | 63.418 | 1.00 | 67.46 | Z | O |
| ATOM | 12698 | NE2 | GLN | Z | 171 | −13.720 | −13.715 | 63.425 | 1.00 | 70.11 | Z | N |
| ATOM | 12699 | C | GLN | Z | 171 | −16.207 | −15.690 | 59.203 | 1.00 | 44.32 | Z | C |
| ATOM | 12700 | O | GLN | Z | 171 | −17.355 | −15.635 | 58.749 | 1.00 | 37.96 | Z | O |
| ATOM | 12701 | N | SER | Z | 172 | −15.281 | −16.547 | 58.776 | 1.00 | 45.40 | Z | N |
| ATOM | 12702 | CA | SER | Z | 172 | −15.522 | −17.562 | 57.754 | 1.00 | 43.63 | Z | C |
| ATOM | 12703 | CB | SER | Z | 172 | −16.089 | −18.851 | 58.376 | 1.00 | 46.28 | Z | C |
| ATOM | 12704 | OG | SER | Z | 172 | −17.194 | −18.601 | 59.230 | 1.00 | 48.13 | Z | O |
| ATOM | 12705 | C | SER | Z | 172 | −16.359 | −17.100 | 56.567 | 1.00 | 43.99 | Z | C |
| ATOM | 12706 | O | SER | Z | 172 | −17.475 | −17.586 | 56.345 | 1.00 | 48.58 | Z | O |
| ATOM | 12707 | N | ASP | Z | 173 | −15.816 | −16.144 | 55.819 | 1.00 | 40.11 | Z | N |
| ATOM | 12708 | CA | ASP | Z | 173 | −16.458 | −15.594 | 54.617 | 1.00 | 39.95 | Z | C |
| ATOM | 12709 | CB | ASP | Z | 173 | −16.823 | −16.718 | 53.623 | 1.00 | 38.64 | Z | C |
| ATOM | 12710 | CG | ASP | Z | 173 | −15.617 | −17.538 | 53.192 | 0.00 | 38.15 | Z | C |
| ATOM | 12711 | OD1 | ASP | Z | 173 | −14.539 | −16.951 | 52.956 | 0.00 | 37.85 | Z | O |
| ATOM | 12712 | OD2 | ASP | Z | 173 | −15.751 | −18.775 | 53.085 | 0.00 | 37.90 | Z | O |
| ATOM | 12713 | C | ASP | Z | 173 | −17.658 | −14.660 | 54.812 | 1.00 | 37.80 | Z | C |
| ATOM | 12714 | O | ASP | Z | 173 | −18.068 | −13.975 | 53.874 | 1.00 | 42.31 | Z | O |
| ATOM | 12715 | N | LEU | Z | 174 | −18.211 | −14.601 | 56.015 | 1.00 | 34.46 | Z | N |
| ATOM | 12716 | CA | LEU | Z | 174 | −19.356 | −13.723 | 56.247 | 1.00 | 32.08 | Z | C |
| ATOM | 12717 | CB | LEU | Z | 174 | −20.594 | −14.547 | 56.662 | 1.00 | 26.91 | Z | C |
| ATOM | 12718 | CG | LEU | Z | 174 | −21.077 | −15.574 | 55.614 | 1.00 | 24.65 | Z | C |
| ATOM | 12719 | CD1 | LEU | Z | 174 | −21.946 | −16.649 | 56.274 | 1.00 | 21.96 | Z | C |
| ATOM | 12720 | CD2 | LEU | Z | 174 | −21.804 | −14.879 | 54.446 | 1.00 | 19.33 | Z | C |
| ATOM | 12721 | C | LEU | Z | 174 | −19.019 | −12.655 | 57.280 | 1.00 | 32.37 | Z | C |
| ATOM | 12722 | O | LEU | Z | 174 | −18.090 | −12.818 | 58.073 | 1.00 | 33.24 | Z | O |
| ATOM | 12723 | N | TYR | Z | 175 | −19.764 | −11.556 | 57.250 | 1.00 | 27.72 | Z | N |
| ATOM | 12724 | CA | TYR | Z | 175 | −19.538 | −10.446 | 58.168 | 1.00 | 30.36 | Z | C |
| ATOM | 12725 | CB | TYR | Z | 175 | −19.800 | −9.096 | 57.483 | 1.00 | 32.30 | Z | C |
| ATOM | 12726 | CG | TYR | Z | 175 | −18.818 | −8.728 | 56.383 | 1.00 | 35.58 | Z | C |
| ATOM | 12727 | CD1 | TYR | Z | 175 | −17.527 | −8.291 | 56.686 | 1.00 | 35.51 | Z | C |
| ATOM | 12728 | CE1 | TYR | Z | 175 | −16.631 | −7.934 | 55.675 | 1.00 | 38.11 | Z | C |
| ATOM | 12729 | CD2 | TYR | Z | 175 | −19.189 | −8.799 | 55.034 | 1.00 | 35.30 | Z | C |
| ATOM | 12730 | CE2 | TYR | Z | 175 | −18.304 | −8.442 | 54.016 | 1.00 | 34.95 | Z | C |
| ATOM | 12731 | CZ | TYR | Z | 175 | −17.029 | −8.011 | 54.343 | 1.00 | 41.21 | Z | C |
| ATOM | 12732 | OH | TYR | Z | 175 | −16.156 | −7.646 | 53.345 | 1.00 | 42.57 | Z | O |
| ATOM | 12733 | C | TYR | Z | 175 | −20.363 | −10.508 | 59.444 | 1.00 | 29.52 | Z | C |
| ATOM | 12734 | O | TYR | Z | 175 | −21.504 | −10.953 | 59.442 | 1.00 | 27.04 | Z | O |
| ATOM | 12735 | N | THR | Z | 176 | −19.752 | −10.031 | 60.525 | 1.00 | 25.36 | Z | N |
| ATOM | 12736 | CA | THR | Z | 176 | −20.351 | −9.958 | 61.842 | 1.00 | 19.70 | Z | C |
| ATOM | 12737 | CB | THR | Z | 176 | −19.772 | −11.028 | 62.789 | 1.00 | 21.66 | Z | C |
| ATOM | 12738 | OG1 | THR | Z | 176 | −20.226 | −12.330 | 62.392 | 1.00 | 29.17 | Z | O |
| ATOM | 12739 | CG2 | THR | Z | 176 | −20.211 | −10.766 | 64.216 | 1.00 | 21.89 | Z | C |
| ATOM | 12740 | C | THR | Z | 176 | −20.030 | −8.562 | 62.401 | 1.00 | 22.87 | Z | C |
| ATOM | 12741 | O | THR | Z | 176 | −18.949 | −8.011 | 62.183 | 1.00 | 24.08 | Z | O |
| ATOM | 12742 | N | LEU | Z | 177 | −20.952 | −8.028 | 63.181 | 1.00 | 20.21 | Z | N |
| ATOM | 12743 | CA | LEU | Z | 177 | −20.799 | −6.709 | 63.762 | 1.00 | 19.62 | Z | C |
| ATOM | 12744 | CB | LEU | Z | 177 | −21.262 | −5.702 | 62.697 | 1.00 | 14.43 | Z | C |
| ATOM | 12745 | CG | LEU | Z | 177 | −21.948 | −4.349 | 62.856 | 1.00 | 21.97 | Z | C |
| ATOM | 12746 | CD1 | LEU | Z | 177 | −21.973 | −3.675 | 61.512 | 1.00 | 13.32 | Z | C |
| ATOM | 12747 | CD2 | LEU | Z | 177 | −23.354 | −4.469 | 63.374 | 1.00 | 14.21 | Z | C |
| ATOM | 12748 | C | LEU | Z | 177 | −21.668 | −6.675 | 65.023 | 1.00 | 22.24 | Z | C |
| ATOM | 12749 | O | LEU | Z | 177 | −22.432 | −7.601 | 65.286 | 1.00 | 19.57 | Z | O |
| ATOM | 12750 | N | SER | Z | 178 | −21.545 | −5.634 | 65.827 | 1.00 | 18.02 | Z | N |
| ATOM | 12751 | CA | SER | Z | 178 | −22.382 | −5.555 | 67.000 | 1.00 | 21.35 | Z | C |
| ATOM | 12752 | CB | SER | Z | 178 | −21.826 | −6.374 | 68.163 | 1.00 | 23.52 | Z | C |
| ATOM | 12753 | OG | SER | Z | 178 | −20.442 | −6.182 | 68.291 | 1.00 | 36.20 | Z | O |
| ATOM | 12754 | C | SER | Z | 178 | −22.609 | −4.128 | 67.392 | 1.00 | 19.22 | Z | C |
| ATOM | 12755 | O | SER | Z | 178 | −21.810 | −3.259 | 67.100 | 1.00 | 23.76 | Z | O |
| ATOM | 12756 | N | SER | Z | 179 | −23.794 | −3.881 | 67.923 | 1.00 | 19.61 | Z | N |
| ATOM | 12757 | CA | SER | Z | 179 | −24.172 | −2.564 | 68.371 | 1.00 | 15.66 | Z | C |
| ATOM | 12758 | CB | SER | Z | 179 | −25.319 | −2.017 | 67.532 | 1.00 | 14.51 | Z | C |
| ATOM | 12759 | OG | SER | Z | 179 | −25.808 | −0.786 | 68.047 | 1.00 | 21.03 | Z | O |
| ATOM | 12760 | C | SER | Z | 179 | −24.608 | −2.697 | 69.818 | 1.00 | 19.76 | Z | C |
| ATOM | 12761 | O | SER | Z | 179 | −25.016 | −3.775 | 70.260 | 1.00 | 23.29 | Z | O |
| ATOM | 12762 | N | SER | Z | 180 | −24.505 | −1.604 | 70.558 | 1.00 | 17.26 | Z | N |
| ATOM | 12763 | CA | SER | Z | 180 | −24.902 | −1.608 | 71.953 | 1.00 | 20.32 | Z | C |
| ATOM | 12764 | CB | SER | Z | 180 | −23.685 | −1.833 | 72.868 | 1.00 | 13.20 | Z | C |
| ATOM | 12765 | OG | SER | Z | 180 | −22.905 | −0.653 | 72.982 | 1.00 | 28.47 | Z | O |
| ATOM | 12766 | C | SER | Z | 180 | −25.551 | −0.279 | 72.270 | 1.00 | 18.41 | Z | C |
| ATOM | 12767 | O | SER | Z | 180 | −25.422 | 0.693 | 71.526 | 1.00 | 23.95 | Z | O |
| ATOM | 12768 | N | VAL | Z | 181 | −26.280 | −0.253 | 73.369 | 1.00 | 20.41 | Z | N |
| ATOM | 12769 | CA | VAL | Z | 181 | −26.936 | 0.960 | 73.813 | 1.00 | 20.87 | Z | C |
| ATOM | 12770 | CB | VAL | Z | 181 | −28.365 | 1.109 | 73.222 | 1.00 | 24.94 | Z | C |
| ATOM | 12771 | CG1 | VAL | Z | 181 | −29.217 | −0.108 | 73.549 | 1.00 | 18.28 | Z | C |
| ATOM | 12772 | CG2 | VAL | Z | 181 | −29.009 | 2.411 | 73.710 | 1.00 | 20.20 | Z | C |
| ATOM | 12773 | C | VAL | Z | 181 | −26.936 | 0.872 | 75.323 | 1.00 | 26.92 | Z | C |
| ATOM | 12774 | O | VAL | Z | 181 | −27.073 | −0.212 | 75.906 | 1.00 | 25.88 | Z | O |
| ATOM | 12775 | N | THR | Z | 182 | −26.640 | 2.000 | 75.945 | 1.00 | 30.03 | Z | N |
| ATOM | 12776 | CA | THR | Z | 182 | −26.563 | 2.078 | 77.382 | 1.00 | 22.46 | Z | C |
| ATOM | 12777 | CB | THR | Z | 182 | −25.197 | 2.649 | 77.788 | 1.00 | 26.09 | Z | C |
| ATOM | 12778 | OG1 | THR | Z | 182 | −24.229 | 1.598 | 77.709 | 1.00 | 26.76 | Z | O |
| ATOM | 12779 | CG2 | THR | Z | 182 | −25.214 | 3.214 | 79.189 | 1.00 | 27.93 | Z | C |
| ATOM | 12780 | C | THR | Z | 182 | −27.719 | 2.922 | 77.845 | 1.00 | 27.77 | Z | C |
| ATOM | 12781 | O | THR | Z | 182 | −27.878 | 4.056 | 77.395 | 1.00 | 23.89 | Z | O |
| ATOM | 12782 | N | VAL | Z | 183 | −28.566 | 2.327 | 78.685 | 1.00 | 25.64 | Z | N |
| ATOM | 12783 | CA | VAL | Z | 183 | −29.760 | 2.998 | 79.203 | 1.00 | 27.19 | Z | C |
| ATOM | 12784 | CB | VAL | Z | 183 | −31.048 | 2.334 | 78.667 | 1.00 | 23.44 | Z | C |
| ATOM | 12785 | CG1 | VAL | Z | 183 | −31.116 | 2.458 | 77.176 | 1.00 | 21.61 | Z | C |
| ATOM | 12786 | CG2 | VAL | Z | 183 | −31.116 | 0.878 | 79.107 | 1.00 | 19.34 | Z | C |
| ATOM | 12787 | C | VAL | Z | 183 | −29.835 | 2.947 | 80.712 | 1.00 | 28.75 | Z | C |
| ATOM | 12788 | O | VAL | Z | 183 | −29.175 | 2.123 | 81.347 | 1.00 | 32.20 | Z | O |
| ATOM | 12789 | N | THR | Z | 184 | −30.685 | 3.784 | 81.292 | 1.00 | 31.39 | Z | N |
| ATOM | 12790 | CA | THR | Z | 184 | −30.824 | 3.775 | 82.744 | 1.00 | 37.54 | Z | C |
| ATOM | 12791 | CB | THR | Z | 184 | −31.636 | 4.982 | 83.238 | 1.00 | 39.80 | Z | C |
| ATOM | 12792 | OG1 | THR | Z | 184 | −33.034 | 4.703 | 83.124 | 1.00 | 52.63 | Z | O |
| ATOM | 12793 | CG2 | THR | Z | 184 | −31.307 | 6.218 | 82.398 | 1.00 | 40.81 | Z | C |
| ATOM | 12794 | C | THR | Z | 184 | −31.478 | 2.463 | 83.195 | 1.00 | 33.27 | Z | C |
| ATOM | 12795 | O | THR | Z | 184 | −32.343 | 1.929 | 82.507 | 1.00 | 37.02 | Z | O |
| ATOM | 12796 | N | SER | Z | 185 | −31.011 | 1.938 | 84.326 | 1.00 | 37.31 | Z | N |
| ATOM | 12797 | CA | SER | Z | 185 | −31.475 | 0.685 | 84.923 | 1.00 | 32.32 | Z | C |
| ATOM | 12798 | CB | SER | Z | 185 | −30.672 | 0.440 | 86.201 | 1.00 | 36.74 | Z | C |
| ATOM | 12799 | OG | SER | Z | 185 | −30.860 | −0.879 | 86.685 | 1.00 | 52.30 | Z | O |
| ATOM | 12800 | C | SER | Z | 185 | −32.979 | 0.659 | 85.235 | 1.00 | 32.46 | Z | C |
| ATOM | 12801 | O | SER | Z | 185 | −33.609 | −0.399 | 85.301 | 1.00 | 33.89 | Z | O |
| ATOM | 12802 | N | SER | Z | 186 | −33.540 | 1.835 | 85.453 | 1.00 | 28.75 | Z | N |
| ATOM | 12803 | CA | SER | Z | 186 | −34.950 | 1.988 | 85.757 | 1.00 | 31.81 | Z | C |
| ATOM | 12804 | CB | SER | Z | 186 | −35.178 | 3.384 | 86.334 | 1.00 | 33.94 | Z | C |
| ATOM | 12805 | OG | SER | Z | 186 | −34.878 | 4.396 | 85.379 | 1.00 | 35.86 | Z | O |
| ATOM | 12806 | C | SER | Z | 186 | −35.779 | 1.836 | 84.482 | 1.00 | 36.33 | Z | C |
| ATOM | 12807 | O | SER | Z | 186 | −36.989 | 1.628 | 84.536 | 1.00 | 35.94 | Z | O |
| ATOM | 12808 | N | THR | Z | 187 | −35.097 | 1.954 | 83.346 | 1.00 | 32.70 | Z | N |
| ATOM | 12809 | CA | THR | Z | 187 | −35.676 | 1.872 | 82.004 | 1.00 | 32.32 | Z | C |
| ATOM | 12810 | CB | THR | Z | 187 | −34.855 | 2.794 | 81.052 | 1.00 | 32.59 | Z | C |
| ATOM | 12811 | OG1 | THR | Z | 187 | −35.292 | 4.151 | 81.220 | 1.00 | 38.01 | Z | O |
| ATOM | 12812 | CG2 | THR | Z | 187 | −34.958 | 2.386 | 79.590 | 1.00 | 41.16 | Z | C |
| ATOM | 12813 | C | THR | Z | 187 | −35.814 | 0.451 | 81.423 | 1.00 | 31.16 | Z | C |
| ATOM | 12814 | O | THR | Z | 187 | −36.745 | 0.165 | 80.677 | 1.00 | 32.02 | Z | O |
| ATOM | 12815 | N | TRP | Z | 188 | −34.893 | −0.430 | 81.775 | 1.00 | 29.66 | Z | N |
| ATOM | 12816 | CA | TRP | Z | 188 | −34.919 | −1.796 | 81.291 | 1.00 | 27.64 | Z | C |
| ATOM | 12817 | CB | TRP | Z | 188 | −33.859 | −1.966 | 80.194 | 1.00 | 23.39 | Z | C |
| ATOM | 12818 | CG | TRP | Z | 188 | −33.797 | −3.347 | 79.646 | 1.00 | 18.92 | Z | C |
| ATOM | 12819 | CD2 | TRP | Z | 188 | −33.019 | −4.433 | 80.151 | 1.00 | 14.33 | Z | C |
| ATOM | 12820 | CE2 | TRP | Z | 188 | −33.294 | −5.559 | 79.339 | 1.00 | 17.05 | Z | C |
| ATOM | 12821 | CE3 | TRP | Z | 188 | −32.111 | −4.568 | 81.208 | 1.00 | 16.97 | Z | C |
| ATOM | 12822 | CD1 | TRP | Z | 188 | −34.497 | −3.837 | 78.573 | 1.00 | 16.42 | Z | C |
| ATOM | 12823 | NE1 | TRP | Z | 188 | −34.198 | −5.167 | 78.387 | 1.00 | 17.74 | Z | N |
| ATOM | 12824 | CZ2 | TRP | Z | 188 | −32.692 | −6.810 | 79.552 | 1.00 | 19.31 | Z | C |
| ATOM | 12825 | CZ3 | TRP | Z | 188 | −31.509 | −5.816 | 81.426 | 1.00 | 20.56 | Z | C |
| ATOM | 12826 | CH2 | TRP | Z | 188 | −31.804 | −6.920 | 80.596 | 1.00 | 20.54 | Z | C |
| ATOM | 12827 | C | TRP | Z | 188 | −34.588 | −2.687 | 82.480 | 1.00 | 31.11 | Z | C |
| ATOM | 12828 | O | TRP | Z | 188 | −33.647 | −2.407 | 83.207 | 1.00 | 37.05 | Z | O |
| ATOM | 12829 | N | PRO | Z | 189 | −35.259 | −3.839 | 82.618 | 1.00 | 37.97 | Z | N |
| ATOM | 12830 | CD | PRO | Z | 189 | −34.851 | −4.750 | 83.710 | 1.00 | 34.86 | Z | C |
| ATOM | 12831 | CA | PRO | Z | 189 | −36.290 | −4.461 | 81.779 | 1.00 | 38.27 | Z | C |
| ATOM | 12832 | CB | PRO | Z | 189 | −36.048 | −5.942 | 82.014 | 1.00 | 38.94 | Z | C |
| ATOM | 12833 | CG | PRO | Z | 189 | −35.741 | −5.962 | 83.493 | 1.00 | 39.93 | Z | C |
| ATOM | 12834 | C | PRO | Z | 189 | −37.730 | −4.091 | 82.116 | 1.00 | 39.28 | Z | C |
| ATOM | 12835 | O | PRO | Z | 189 | −38.664 | −4.771 | 81.681 | 1.00 | 41.44 | Z | O |
| ATOM | 12836 | N | SER | Z | 190 | −37.910 | −3.030 | 82.891 | 1.00 | 38.80 | Z | N |
| ATOM | 12837 | CA | SER | Z | 190 | −39.249 | −2.594 | 83.264 | 1.00 | 41.58 | Z | C |
| ATOM | 12838 | CB | SER | Z | 190 | −39.176 | −1.355 | 84.145 | 1.00 | 42.86 | Z | C |
| ATOM | 12839 | OG | SER | Z | 190 | −38.750 | −0.235 | 83.390 | 1.00 | 50.83 | Z | O |
| ATOM | 12840 | C | SER | Z | 190 | −40.003 | −2.267 | 81.987 | 1.00 | 43.15 | Z | C |
| ATOM | 12841 | O | SER | Z | 190 | −41.199 | −2.557 | 81.857 | 1.00 | 50.42 | Z | O |
| ATOM | 12842 | N | GLN | Z | 191 | −39.287 | −1.622 | 81.069 | 1.00 | 42.67 | Z | N |
| ATOM | 12843 | CA | GLN | Z | 191 | −39.803 | −1.248 | 79.756 | 1.00 | 40.68 | Z | C |
| ATOM | 12844 | CB | GLN | Z | 191 | −39.811 | 0.269 | 79.581 | 1.00 | 40.37 | Z | C |
| ATOM | 12845 | CG | GLN | Z | 191 | −40.786 | 0.932 | 80.551 | 1.00 | 43.44 | Z | C |
| ATOM | 12846 | CD | GLN | Z | 191 | −40.961 | 2.411 | 80.331 | 1.00 | 41.80 | Z | C |
| ATOM | 12847 | OE1 | GLN | Z | 191 | −40.159 | 3.059 | 79.660 | 1.00 | 46.68 | Z | O |
| ATOM | 12848 | NE2 | GLN | Z | 191 | −42.014 | 2.963 | 80.920 | 1.00 | 46.49 | Z | N |
| ATOM | 12849 | C | GLN | Z | 191 | −38.960 | −1.970 | 78.698 | 1.00 | 42.07 | Z | C |
| ATOM | 12850 | O | GLN | Z | 191 | −37.945 | −2.595 | 79.026 | 1.00 | 44.51 | Z | O |
| ATOM | 12851 | N | SER | Z | 192 | −39.382 | −1.909 | 77.441 | 1.00 | 41.25 | Z | N |
| ATOM | 12852 | CA | SER | Z | 192 | −38.694 | −2.647 | 76.388 | 1.00 | 36.18 | Z | C |
| ATOM | 12853 | CB | SER | Z | 192 | −39.713 | −3.254 | 75.417 | 1.00 | 39.02 | Z | C |
| ATOM | 12854 | OG | SER | Z | 192 | −40.461 | −2.231 | 74.786 | 1.00 | 38.78 | Z | O |
| ATOM | 12855 | C | SER | Z | 192 | −37.632 | −1.930 | 75.600 | 1.00 | 32.61 | Z | C |
| ATOM | 12856 | O | SER | Z | 192 | −37.688 | −0.710 | 75.383 | 1.00 | 32.45 | Z | O |
| ATOM | 12857 | N | VAL | Z | 193 | −36.656 | −2.725 | 75.166 | 1.00 | 27.64 | Z | N |
| ATOM | 12858 | CA | VAL | Z | 193 | −35.553 | −2.239 | 74.359 | 1.00 | 23.36 | Z | C |
| ATOM | 12859 | CB | VAL | Z | 193 | −34.243 | −2.095 | 75.179 | 1.00 | 25.03 | Z | C |
| ATOM | 12860 | CG1 | VAL | Z | 193 | −33.049 | −1.833 | 74.257 | 1.00 | 22.28 | Z | C |
| ATOM | 12861 | CG2 | VAL | Z | 193 | −34.383 | −0.950 | 76.155 | 1.00 | 14.86 | Z | C |
| ATOM | 12862 | C | VAL | Z | 193 | −35.384 | −3.187 | 73.186 | 1.00 | 22.24 | Z | C |
| ATOM | 12863 | O | VAL | Z | 193 | −35.130 | −4.383 | 73.334 | 1.00 | 21.62 | Z | O |
| ATOM | 12864 | N | THR | Z | 194 | −35.542 | −2.638 | 72.000 | 1.00 | 20.15 | Z | N |
| ATOM | 12865 | CA | THR | Z | 194 | −35.427 | −3.451 | 70.817 | 1.00 | 23.98 | Z | C |
| ATOM | 12866 | CB | THR | Z | 194 | −36.810 | −3.545 | 70.117 | 1.00 | 23.45 | Z | C |
| ATOM | 12867 | OG1 | THR | Z | 194 | −37.730 | −4.164 | 71.009 | 1.00 | 23.98 | Z | O |
| ATOM | 12868 | CG2 | THR | Z | 194 | −36.735 | −4.357 | 68.815 | 1.00 | 26.26 | Z | C |
| ATOM | 12869 | C | THR | Z | 194 | −34.444 | −2.848 | 69.836 | 1.00 | 22.73 | Z | C |
| ATOM | 12870 | O | THR | Z | 194 | −34.440 | −1.629 | 69.636 | 1.00 | 25.45 | Z | O |
| ATOM | 12871 | N | CYS | Z | 195 | −33.585 | −3.684 | 69.253 | 0.50 | 21.53 | Z | N |
| ATOM | 12872 | CA | CYS | Z | 195 | −32.628 | −3.209 | 68.256 | 0.50 | 21.95 | Z | C |
| ATOM | 12873 | C | CYS | Z | 195 | −33.191 | −3.582 | 66.877 | 0.50 | 23.97 | Z | C |
| ATOM | 12874 | O | CYS | Z | 195 | −33.552 | −4.735 | 66.623 | 0.50 | 23.88 | Z | O |
| ATOM | 12875 | CB | CYS | Z | 195 | −31.228 | −3.806 | 68.478 | 0.50 | 21.91 | Z | C |
| ATOM | 12876 | SG | CYS | Z | 195 | −30.801 | −5.314 | 67.546 | 0.50 | 26.20 | Z | S |
| ATOM | 12877 | N | ASN | Z | 196 | −33.315 | −2.580 | 66.016 | 1.00 | 22.46 | Z | N |
| ATOM | 12878 | CA | ASN | Z | 196 | −33.855 | −2.750 | 64.676 | 1.00 | 22.32 | Z | C |
| ATOM | 12879 | CB | ASN | Z | 196 | −34.720 | −1.549 | 64.317 | 1.00 | 19.12 | Z | C |
| ATOM | 12880 | CG | ASN | Z | 196 | −35.553 | −1.073 | 65.485 | 1.00 | 29.98 | Z | C |
| ATOM | 12881 | OD1 | ASN | Z | 196 | −35.304 | 0.000 | 66.036 | 1.00 | 31.00 | Z | O |
| ATOM | 12882 | ND2 | ASN | Z | 196 | −36.544 | −1.869 | 65.880 | 1.00 | 26.21 | Z | N |
| ATOM | 12883 | C | ASN | Z | 196 | −32.700 | −2.856 | 63.692 | 1.00 | 25.21 | Z | C |
| ATOM | 12884 | O | ASN | Z | 196 | −31.916 | −1.903 | 63.547 | 1.00 | 22.25 | Z | O |
| ATOM | 12885 | N | VAL | Z | 197 | −32.611 | −4.006 | 63.017 | 1.00 | 23.49 | Z | N |
| ATOM | 12886 | CA | VAL | Z | 197 | −31.542 | −4.279 | 62.043 | 1.00 | 21.79 | Z | C |
| ATOM | 12887 | CB | VAL | Z | 197 | −30.838 | −5.627 | 62.362 | 1.00 | 21.90 | Z | C |
| ATOM | 12888 | CG1 | VAL | Z | 197 | −29.788 | −5.970 | 61.286 | 1.00 | 17.65 | Z | C |
| ATOM | 12889 | CG2 | VAL | Z | 197 | −30.211 | −5.586 | 63.737 | 1.00 | 14.03 | Z | C |
| ATOM | 12890 | C | VAL | Z | 197 | −32.053 | −4.346 | 60.605 | 1.00 | 20.53 | Z | C |
| ATOM | 12891 | O | VAL | Z | 197 | −33.049 | −5.005 | 60.324 | 1.00 | 29.69 | Z | O |
| ATOM | 12892 | N | ALA | Z | 198 | −31.358 | −3.678 | 59.695 | 1.00 | 21.87 | Z | N |
| ATOM | 12893 | CA | ALA | Z | 198 | −31.738 | −3.679 | 58.276 | 1.00 | 26.44 | Z | C |
| ATOM | 12894 | CB | ALA | Z | 198 | −32.309 | −2.292 | 57.849 | 1.00 | 16.04 | Z | C |
| ATOM | 12895 | C | ALA | Z | 198 | −30.528 | −4.044 | 57.416 | 1.00 | 20.88 | Z | C |
| ATOM | 12896 | O | ALA | Z | 198 | −29.423 | −3.597 | 57.681 | 1.00 | 20.50 | Z | O |
| ATOM | 12897 | N | HIS | Z | 199 | −30.751 | −4.884 | 56.410 | 1.00 | 26.23 | Z | N |
| ATOM | 12898 | CA | HIS | Z | 199 | −29.711 | −5.329 | 55.480 | 1.00 | 21.64 | Z | C |
| ATOM | 12899 | CB | HIS | Z | 199 | −29.465 | −6.813 | 55.675 | 1.00 | 23.14 | Z | C |
| ATOM | 12900 | CG | HIS | Z | 199 | −28.424 | −7.383 | 54.766 | 1.00 | 25.46 | Z | C |
| ATOM | 12901 | CD2 | HIS | Z | 199 | −28.391 | −8.541 | 54.069 | 1.00 | 19.31 | Z | C |
| ATOM | 12902 | ND1 | HIS | Z | 199 | −27.216 | −6.762 | 54.534 | 1.00 | 29.64 | Z | N |
| ATOM | 12903 | CE1 | HIS | Z | 199 | −26.483 | −7.515 | 53.735 | 1.00 | 31.16 | Z | C |
| ATOM | 12904 | NE2 | HIS | Z | 199 | −27.175 | −8.600 | 53.439 | 1.00 | 30.28 | Z | N |
| ATOM | 12905 | C | HIS | Z | 199 | −30.271 | −5.061 | 54.088 | 1.00 | 23.51 | Z | C |
| ATOM | 12906 | O | HIS | Z | 199 | −30.911 | −5.911 | 53.485 | 1.00 | 27.02 | Z | O |
| ATOM | 12907 | N | PRO | Z | 200 | −29.985 | −3.883 | 53.537 | 1.00 | 26.39 | Z | N |
| ATOM | 12908 | CD | PRO | Z | 200 | −29.036 | −2.888 | 54.069 | 1.00 | 24.59 | Z | C |
| ATOM | 12909 | CA | PRO | Z | 200 | −30.474 | −3.483 | 52.218 | 1.00 | 25.21 | Z | C |
| ATOM | 12910 | CB | PRO | Z | 200 | −29.777 | −2.132 | 51.996 | 1.00 | 28.90 | Z | C |
| ATOM | 12911 | CG | PRO | Z | 200 | −29.519 | −1.622 | 53.406 | 1.00 | 24.77 | Z | C |
| ATOM | 12912 | C | PRO | Z | 200 | −30.253 | −4.450 | 51.043 | 1.00 | 29.52 | Z | C |
| ATOM | 12913 | O | PRO | Z | 200 | −31.152 | −4.662 | 50.232 | 1.00 | 31.71 | Z | O |
| ATOM | 12914 | N | ALA | Z | 201 | −29.074 | −5.043 | 50.947 | 1.00 | 29.58 | Z | N |
| ATOM | 12915 | CA | ALA | Z | 201 | −28.791 | −5.949 | 49.834 | 1.00 | 29.57 | Z | C |
| ATOM | 12916 | CB | ALA | Z | 201 | −27.374 | −6.468 | 49.918 | 1.00 | 18.96 | Z | C |
| ATOM | 12917 | C | ALA | Z | 201 | −29.776 | −7.105 | 49.723 | 1.00 | 30.13 | Z | C |
| ATOM | 12918 | O | ALA | Z | 201 | −30.068 | −7.584 | 48.615 | 1.00 | 27.92 | Z | O |
| ATOM | 12919 | N | SER | Z | 202 | −30.288 | −7.544 | 50.870 | 1.00 | 30.04 | Z | N |
| ATOM | 12920 | CA | SER | Z | 202 | −31.244 | −8.644 | 50.913 | 1.00 | 24.95 | Z | C |
| ATOM | 12921 | CB | SER | Z | 202 | −30.808 | −9.689 | 51.940 | 1.00 | 28.09 | Z | C |
| ATOM | 12922 | OG | SER | Z | 202 | −31.044 | −9.241 | 53.269 | 1.00 | 29.12 | Z | O |
| ATOM | 12923 | C | SER | Z | 202 | −32.648 | −8.157 | 51.255 | 1.00 | 26.08 | Z | C |
| ATOM | 12924 | O | SER | Z | 202 | −33.527 | −8.969 | 51.488 | 1.00 | 26.25 | Z | O |
| ATOM | 12925 | N | SER | Z | 203 | −32.843 | −6.837 | 51.279 | 1.00 | 22.31 | Z | N |
| ATOM | 12926 | CA | SER | Z | 203 | −34.128 | −6.209 | 51.603 | 1.00 | 28.75 | Z | C |
| ATOM | 12927 | CB | SER | Z | 203 | −35.070 | −6.229 | 50.392 | 1.00 | 27.72 | Z | C |
| ATOM | 12928 | OG | SER | Z | 203 | −36.178 | −5.363 | 50.589 | 0.00 | 27.75 | Z | O |
| ATOM | 12929 | C | SER | Z | 203 | −34.781 | −6.854 | 52.831 | 1.00 | 28.03 | Z | C |
| ATOM | 12930 | O | SER | Z | 203 | −35.979 | −7.107 | 52.869 | 1.00 | 32.09 | Z | O |
| ATOM | 12931 | N | THR | Z | 204 | −33.962 | −7.058 | 53.852 | 1.00 | 30.03 | Z | N |
| ATOM | 12932 | CA | THR | Z | 204 | −34.369 | −7.687 | 55.094 | 1.00 | 28.56 | Z | C |
| ATOM | 12933 | CB | THR | Z | 204 | −33.485 | −8.944 | 55.341 | 1.00 | 29.46 | Z | C |
| ATOM | 12934 | OG1 | THR | Z | 204 | −33.743 | −9.920 | 54.322 | 1.00 | 34.59 | Z | O |
| ATOM | 12935 | CG2 | THR | Z | 204 | −33.733 | −9.555 | 56.710 | 1.00 | 32.32 | Z | C |
| ATOM | 12936 | C | THR | Z | 204 | −34.263 | −6.752 | 56.307 | 1.00 | 28.08 | Z | C |
| ATOM | 12937 | O | THR | Z | 204 | −33.296 | −6.004 | 56.461 | 1.00 | 32.70 | Z | O |
| ATOM | 12938 | N | LYS | Z | 205 | −35.282 | −6.793 | 57.150 | 1.00 | 24.85 | Z | N |
| ATOM | 12939 | CA | LYS | Z | 205 | −35.326 | −6.020 | 58.382 | 1.00 | 26.11 | Z | C |
| ATOM | 12940 | CB | LYS | Z | 205 | −36.402 | −4.931 | 58.326 | 1.00 | 28.10 | Z | C |
| ATOM | 12941 | CG | LYS | Z | 205 | −36.130 | −3.744 | 57.407 | 1.00 | 32.33 | Z | C |
| ATOM | 12942 | CD | LYS | Z | 205 | −37.326 | −2.774 | 57.469 | 1.00 | 38.90 | Z | C |
| ATOM | 12943 | CE | LYS | Z | 205 | −37.056 | −1.414 | 56.816 | 1.00 | 43.06 | Z | C |
| ATOM | 12944 | NZ | LYS | Z | 205 | −36.956 | −1.474 | 55.330 | 1.00 | 42.82 | Z | N |
| ATOM | 12945 | C | LYS | Z | 205 | −35.681 | −7.009 | 59.490 | 1.00 | 24.50 | Z | C |
| ATOM | 12946 | O | LYS | Z | 205 | −36.565 | −7.846 | 59.318 | 1.00 | 26.41 | Z | O |
| ATOM | 12947 | N | VAL | Z | 206 | −34.938 | −6.968 | 60.589 | 1.00 | 24.20 | Z | N |
| ATOM | 12948 | CA | VAL | Z | 206 | −35.199 | −7.839 | 61.735 | 1.00 | 22.23 | Z | C |
| ATOM | 12949 | CB | VAL | Z | 206 | −34.083 | −8.883 | 61.933 | 1.00 | 26.41 | Z | C |
| ATOM | 12950 | CG1 | VAL | Z | 206 | −34.513 | −9.914 | 62.967 | 1.00 | 26.15 | Z | C |
| ATOM | 12951 | CG2 | VAL | Z | 206 | −33.703 | −9.552 | 60.623 | 1.00 | 23.58 | Z | C |
| ATOM | 12952 | C | VAL | Z | 206 | −35.234 | −6.974 | 63.005 | 1.00 | 26.53 | Z | C |
| ATOM | 12953 | O | VAL | Z | 206 | −34.537 | −5.958 | 63.109 | 1.00 | 30.51 | Z | O |
| ATOM | 12954 | N | ASP | Z | 207 | −36.064 | −7.366 | 63.958 | 1.00 | 29.91 | Z | N |
| ATOM | 12955 | CA | ASP | Z | 207 | −36.180 | −6.668 | 65.236 | 1.00 | 31.71 | Z | C |
| ATOM | 12956 | CB | ASP | Z | 207 | −37.608 | −6.177 | 65.461 | 1.00 | 36.16 | Z | C |
| ATOM | 12957 | CG | ASP | Z | 207 | −37.918 | −4.902 | 64.721 | 1.00 | 40.31 | Z | C |
| ATOM | 12958 | OD1 | ASP | Z | 207 | −36.974 | −4.194 | 64.319 | 1.00 | 40.11 | Z | O |
| ATOM | 12959 | OD2 | ASP | Z | 207 | −39.125 | −4.600 | 64.557 | 1.00 | 49.25 | Z | O |
| ATOM | 12960 | C | ASP | Z | 207 | −35.845 | −7.659 | 66.342 | 1.00 | 33.98 | Z | C |
| ATOM | 12961 | O | ASP | Z | 207 | −36.395 | −8.759 | 66.381 | 1.00 | 36.48 | Z | O |
| ATOM | 12962 | N | LYS | Z | 208 | −34.928 | −7.304 | 67.227 | 1.00 | 31.64 | Z | N |
| ATOM | 12963 | CA | LYS | Z | 208 | −34.611 | −8.221 | 68.303 | 1.00 | 25.63 | Z | C |
| ATOM | 12964 | CB | LYS | Z | 208 | −33.168 | −8.698 | 68.214 | 1.00 | 27.33 | Z | C |
| ATOM | 12965 | CG | LYS | Z | 208 | −32.883 | −9.866 | 69.135 | 1.00 | 33.06 | Z | C |
| ATOM | 12966 | CD | LYS | Z | 208 | −33.803 | −11.044 | 68.820 | 1.00 | 33.07 | Z | C |
| ATOM | 12967 | CE | LYS | Z | 208 | −33.919 | −11.984 | 70.013 | 1.00 | 36.10 | Z | C |
| ATOM | 12968 | NZ | LYS | Z | 208 | −34.871 | −13.120 | 69.795 | 1.00 | 40.22 | Z | N |
| ATOM | 12969 | C | LYS | Z | 208 | −34.855 | −7.533 | 69.628 | 1.00 | 28.42 | Z | C |
| ATOM | 12970 | O | LYS | Z | 208 | −34.298 | −6.463 | 69.891 | 1.00 | 27.36 | Z | O |
| ATOM | 12971 | N | LYS | Z | 209 | −35.734 | −8.127 | 70.429 | 1.00 | 27.98 | Z | N |
| ATOM | 12972 | CA | LYS | Z | 209 | −36.068 | −7.607 | 71.744 | 1.00 | 30.73 | Z | C |
| ATOM | 12973 | CB | LYS | Z | 209 | −37.520 | −7.926 | 72.093 | 1.00 | 32.06 | Z | C |
| ATOM | 12974 | CG | LYS | Z | 209 | −37.996 | −7.416 | 73.465 | 1.00 | 40.77 | Z | C |
| ATOM | 12975 | CD | LYS | Z | 209 | −39.384 | −7.999 | 73.795 | 1.00 | 48.07 | Z | C |
| ATOM | 12976 | CE | LYS | Z | 209 | −39.898 | −7.617 | 75.191 | 1.00 | 50.15 | Z | C |
| ATOM | 12977 | NZ | LYS | Z | 209 | −41.218 | −8.269 | 75.508 | 1.00 | 45.90 | Z | N |
| ATOM | 12978 | C | LYS | Z | 209 | −35.137 | −8.211 | 72.788 | 1.00 | 30.94 | Z | C |
| ATOM | 12979 | O | LYS | Z | 209 | −35.032 | −9.436 | 72.933 | 1.00 | 31.39 | Z | O |
| ATOM | 12980 | N | ILE | Z | 210 | −34.487 | −7.334 | 73.540 | 1.00 | 30.03 | Z | N |
| ATOM | 12981 | CA | ILE | Z | 210 | −33.556 | −7.750 | 74.578 | 1.00 | 28.19 | Z | C |
| ATOM | 12982 | CB | ILE | Z | 210 | −32.520 | −6.646 | 74.830 | 1.00 | 27.87 | Z | C |
| ATOM | 12983 | CG2 | ILE | Z | 210 | −31.429 | −7.156 | 75.765 | 1.00 | 25.81 | Z | C |
| ATOM | 12984 | CG1 | ILE | Z | 210 | −31.992 | −6.096 | 73.497 | 1.00 | 22.06 | Z | C |
| ATOM | 12985 | CD1 | ILE | Z | 210 | −31.541 | −7.149 | 72.524 | 1.00 | 15.51 | Z | C |
| ATOM | 12986 | C | ILE | Z | 210 | −34.276 | −8.030 | 75.897 | 1.00 | 32.41 | Z | C |
| ATOM | 12987 | O | ILE | Z | 210 | −34.828 | −7.114 | 76.519 | 1.00 | 32.45 | Z | O |
| ATOM | 12988 | N | VAL | Z | 211 | −34.286 | −9.290 | 76.318 | 1.00 | 33.10 | Z | N |
| ATOM | 12989 | CA | VAL | Z | 211 | −34.932 | −9.639 | 77.577 | 1.00 | 36.29 | Z | C |
| ATOM | 12990 | CB | VAL | Z | 211 | −36.099 | −10.677 | 77.410 | 1.00 | 38.36 | Z | C |
| ATOM | 12991 | CG1 | VAL | Z | 211 | −37.024 | −10.265 | 76.286 | 1.00 | 39.64 | Z | C |
| ATOM | 12992 | CG2 | VAL | Z | 211 | −35.563 | −12.079 | 77.207 | 1.00 | 42.47 | Z | C |
| ATOM | 12993 | C | VAL | Z | 211 | −33.902 | −10.193 | 78.545 | 1.00 | 34.61 | Z | C |
| ATOM | 12994 | O | VAL | Z | 211 | −32.922 | −10.824 | 78.138 | 1.00 | 33.84 | Z | O |
| ATOM | 12995 | N | PRO | Z | 212 | −34.140 | −10.003 | 79.847 | 1.00 | 32.95 | Z | N |
| ATOM | 12996 | CD | PRO | Z | 212 | −35.375 | −9.465 | 80.447 | 1.00 | 32.54 | Z | C |
| ATOM | 12997 | CA | PRO | Z | 212 | −33.222 | −10.485 | 80.878 | 1.00 | 33.24 | Z | C |
| ATOM | 12998 | CB | PRO | Z | 212 | −33.942 | −10.109 | 82.178 | 1.00 | 33.39 | Z | C |
| ATOM | 12999 | CG | PRO | Z | 212 | −35.398 | −10.160 | 81.788 | 1.00 | 35.45 | Z | C |
| ATOM | 13000 | C | PRO | Z | 212 | −33.014 | −11.976 | 80.766 | 1.00 | 34.07 | Z | C |
| ATOM | 13001 | O | PRO | Z | 212 | −33.974 | −12.726 | 80.643 | 1.00 | 44.16 | Z | O |
| ATOM | 13002 | N | LYS | Z | 213 | −31.756 | −12.401 | 80.758 | 1.00 | 41.18 | Z | N |
| ATOM | 13003 | CA | LYS | Z | 213 | −31.438 | −13.820 | 80.662 | 1.00 | 46.40 | Z | C |
| ATOM | 13004 | CB | LYS | Z | 213 | −30.266 | −14.067 | 79.693 | 1.00 | 45.27 | Z | C |
| ATOM | 13005 | CG | LYS | Z | 213 | −28.933 | −13.459 | 80.102 | 1.00 | 42.68 | Z | C |
| ATOM | 13006 | CD | LYS | Z | 213 | −27.785 | −14.096 | 79.315 | 1.00 | 47.62 | Z | C |
| ATOM | 13007 | CE | LYS | Z | 213 | −27.605 | −15.575 | 79.690 | 1.00 | 48.88 | Z | C |
| ATOM | 13008 | NZ | LYS | Z | 213 | −26.753 | −16.327 | 78.732 | 1.00 | 44.99 | Z | N |
| ATOM | 13009 | C | LYS | Z | 213 | −31.126 | −14.404 | 82.038 | 1.00 | 48.56 | Z | C |
| ATOM | 13010 | O | LYS | Z | 213 | −31.978 | −14.412 | 82.927 | 1.00 | 48.59 | Z | O |
| ATOM | 13013 | O | WAT | L | 1 | 53.403 | 33.509 | −59.024 | 1.00 | 24.71 | L | O |
| ATOM | 13014 | O | WAT | L | Z | 21.370 | 18.099 | −8.008 | 1.00 | 15.31 | L | O |
| ATOM | 13015 | O | WAT | L | 3 | 20.331 | 15.493 | −8.442 | 1.00 | 25.01 | L | O |
| ATOM | 13016 | O | WAT | L | 4 | 15.959 | 0.378 | −17.048 | 1.00 | 26.27 | L | O |
| ATOM | 13017 | O | WAT | L | 5 | 26.737 | 15.708 | −14.718 | 1.00 | 18.36 | L | O |
| ATOM | 13018 | O | WAT | L | 6 | 18.948 | 23.563 | −13.717 | 1.00 | 23.26 | L | O |
| ATOM | 13019 | O | WAT | L | 7 | 46.829 | 42.038 | −40.323 | 1.00 | 39.25 | L | O |
| ATOM | 13020 | O | WAT | L | 8 | 13.289 | 14.799 | 1.467 | 1.00 | 12.22 | L | O |
| ATOM | 13021 | O | WAT | L | 9 | 18.648 | −8.097 | 12.011 | 1.00 | 34.77 | L | O |
| ATOM | 13022 | O | WAT | L | 10 | 60.986 | 52.021 | −42.197 | 1.00 | 34.02 | L | O |
| ATOM | 13023 | O | WAT | L | 11 | 7.736 | 4.164 | −8.440 | 1.00 | 23.90 | L | O |
| ATOM | 13024 | O | WAT | L | 12 | 13.450 | 27.006 | −47.185 | 1.00 | 33.16 | L | O |
| ATOM | 13025 | O | WAT | L | 13 | 1.479 | 5.811 | −8.271 | 1.00 | 38.47 | L | O |
| ATOM | 13026 | O | WAT | L | 14 | 35.304 | 18.235 | −32.341 | 1.00 | 27.83 | L | O |
| ATOM | 13027 | O | WAT | L | 15 | −6.969 | −1.161 | 4.663 | 1.00 | 28.07 | L | O |
| ATOM | 13028 | O | WAT | L | 16 | 10.586 | 28.515 | −44.340 | 1.00 | 22.98 | L | O |
| ATOM | 13029 | O | WAT | L | 17 | 48.756 | 28.665 | 21.404 | 1.00 | 30.73 | L | O |
| ATOM | 13030 | O | WAT | L | 18 | 19.240 | 14.251 | −24.691 | 1.00 | 19.47 | L | O |
| ATOM | 13031 | O | WAT | L | 19 | 17.219 | 5.475 | 8.099 | 1.00 | 10.03 | L | O |
| ATOM | 13032 | O | WAT | L | 20 | 22.985 | −8.582 | 13.375 | 1.00 | 36.83 | L | O |
| ATOM | 13033 | O | WAT | L | 21 | 21.579 | 3.470 | 14.650 | 1.00 | 17.47 | L | O |
| ATOM | 13034 | O | WAT | L | 22 | 21.615 | 14.323 | −28.497 | 1.00 | 21.70 | L | O |
| ATOM | 13035 | O | WAT | L | 23 | 36.058 | 19.464 | −25.157 | 1.00 | 20.42 | L | O |
| ATOM | 13036 | O | WAT | L | 24 | 55.829 | 44.646 | −46.882 | 1.00 | 32.09 | L | O |
| ATOM | 13037 | O | WAT | L | 25 | 58.513 | 45.330 | −45.520 | 1.00 | 30.94 | L | O |
| ATOM | 13038 | O | WAT | L | 26 | 35.597 | 23.624 | −31.825 | 1.00 | 49.55 | L | O |
| ATOM | 13039 | O | WAT | L | 27 | 18.215 | 25.262 | −6.196 | 1.00 | 39.04 | L | O |
| ATOM | 13040 | O | WAT | L | 28 | 32.878 | 36.316 | −55.529 | 1.00 | 43.64 | L | O |
| ATOM | 13041 | O | WAT | L | 29 | 35.938 | 35.764 | −56.222 | 1.00 | 33.59 | L | O |
| ATOM | 13042 | O | WAT | L | 30 | 41.592 | 26.515 | −32.823 | 1.00 | 59.61 | L | O |
| ATOM | 13043 | O | WAT | L | 31 | 49.909 | 70.201 | −52.583 | 1.00 | 35.54 | L | O |
| ATOM | 13044 | O | WAT | L | 32 | −0.429 | 6.183 | −1.274 | 1.00 | 28.63 | L | O |
| ATOM | 13045 | O | WAT | L | 33 | 18.939 | −2.017 | −12.052 | 1.00 | 47.72 | L | O |
| ATOM | 13046 | O | WAT | L | 34 | 19.857 | 18.596 | −31.839 | 1.00 | 22.44 | L | O |
| ATOM | 13047 | O | WAT | L | 35 | 51.830 | 63.436 | −45.041 | 1.00 | 29.09 | L | O |
| ATOM | 13048 | O | WAT | L | 36 | 34.027 | 26.977 | −10.840 | 1.00 | 23.69 | L | O |
| ATOM | 13049 | O | WAT | L | 37 | 11.544 | 10.650 | −19.189 | 1.00 | 32.34 | L | O |
| ATOM | 13050 | O | WAT | L | 38 | 27.799 | 3.620 | 2.322 | 1.00 | 19.49 | L | O |
| ATOM | 13051 | O | WAT | L | 39 | −18.641 | −6.220 | −3.953 | 1.00 | 31.99 | L | O |
| ATOM | 13052 | O | WAT | L | 40 | 5.552 | 15.127 | 5.093 | 1.00 | 22.95 | L | O |
| ATOM | 13053 | O | WAT | L | 41 | 28.664 | 32.029 | −44.204 | 1.00 | 26.68 | L | O |
| ATOM | 13054 | O | WAT | L | 42 | 32.951 | 14.112 | −36.850 | 1.00 | 29.20 | L | O |
| ATOM | 13055 | O | WAT | L | 43 | 10.159 | 13.923 | 4.200 | 1.00 | 31.76 | L | O |
| ATOM | 13056 | O | WAT | L | 44 | 33.453 | 20.845 | −37.031 | 1.00 | 26.91 | L | O |
| ATOM | 13057 | O | WAT | L | 45 | 26.722 | 9.868 | −22.761 | 1.00 | 44.65 | L | O |
| ATOM | 13058 | O | WAT | L | 46 | 49.837 | 28.810 | −49.976 | 1.00 | 24.51 | L | O |
| ATOM | 13059 | O | WAT | L | 47 | 46.564 | 31.021 | −44.906 | 1.00 | 33.85 | L | O |
| ATOM | 13060 | O | WAT | L | 48 | −5.002 | −15.869 | −3.923 | 1.00 | 32.65 | L | O |
| ATOM | 13061 | O | WAT | L | 49 | 53.982 | 60.534 | −49.264 | 1.00 | 26.63 | L | O |
| ATOM | 13062 | O | WAT | L | 50 | 22.584 | 25.140 | −25.058 | 1.00 | 21.96 | L | O |
| ATOM | 13063 | O | WAT | L | 51 | 27.143 | 10.905 | −35.850 | 1.00 | 26.17 | L | O |
| ATOM | 13064 | O | WAT | L | 52 | 26.288 | 22.718 | −37.644 | 1.00 | 28.52 | L | O |
| ATOM | 13065 | O | WAT | L | 53 | 40.693 | 25.616 | −26.852 | 1.00 | 38.15 | L | O |
| ATOM | 13066 | O | WAT | L | 54 | 14.141 | 25.996 | −6.076 | 1.00 | 39.30 | L | O |
| ATOM | 13067 | O | WAT | L | 55 | 50.436 | 48.787 | −25.917 | 1.00 | 36.09 | L | O |
| ATOM | 13068 | O | WAT | L | 56 | 9.281 | 8.180 | 4.814 | 1.00 | 28.60 | L | O |
| ATOM | 13069 | O | WAT | L | 57 | 21.947 | 19.526 | −18.135 | 1.00 | 13.69 | L | O |
| ATOM | 13070 | O | WAT | L | 58 | 41.809 | 50.771 | −47.984 | 1.00 | 55.35 | L | O |
| ATOM | 13071 | O | WAT | L | 59 | 42.596 | 35.351 | −37.149 | 1.00 | 31.88 | L | O |
| ATOM | 13072 | O | WAT | L | 60 | 48.151 | 56.277 | −33.834 | 1.00 | 28.36 | L | O |
| ATOM | 13073 | O | WAT | L | 61 | −13.144 | 17.565 | −15.368 | 1.00 | 26.30 | L | O |
| ATOM | 13074 | O | WAT | L | 62 | 42.678 | 30.869 | −52.606 | 1.00 | 25.49 | L | O |
| ATOM | 13075 | O | WAT | L | 63 | 9.557 | 1.161 | 7.168 | 1.00 | 22.76 | L | O |
| ATOM | 13076 | O | WAT | L | 64 | 55.445 | 52.690 | −55.421 | 1.00 | 46.42 | L | O |
| ATOM | 13077 | O | WAT | L | 65 | 14.131 | 14.685 | −6.808 | 1.00 | 24.27 | L | O |
| ATOM | 13078 | O | WAT | L | 66 | 28.078 | 19.781 | −17.940 | 1.00 | 31.62 | L | O |
| ATOM | 13079 | O | WAT | L | 67 | 24.443 | 34.343 | −22.262 | 1.00 | 29.80 | L | O |
| ATOM | 13080 | O | WAT | L | 68 | 61.607 | 29.695 | −62.455 | 1.00 | 42.57 | L | O |
| ATOM | 13081 | O | WAT | L | 69 | 15.869 | 24.638 | −13.706 | 1.00 | 25.73 | L | O |
| ATOM | 13082 | O | WAT | L | 70 | 15.257 | 27.311 | −15.132 | 1.00 | 38.55 | L | O |
| ATOM | 13083 | O | WAT | L | 71 | 23.705 | 6.869 | −7.164 | 1.00 | 30.04 | L | O |
| ATOM | 13084 | O | WAT | L | 72 | 4.591 | −8.214 | 0.963 | 1.00 | 39.04 | L | O |
| ATOM | 13085 | O | WAT | L | 73 | 53.832 | 34.440 | −42.611 | 1.00 | 33.60 | L | O |
| ATOM | 13086 | O | WAT | L | 74 | −18.832 | 8.220 | 0.538 | 1.00 | 29.85 | L | O |
| ATOM | 13087 | O | WAT | L | 75 | 33.626 | 31.655 | −50.152 | 1.00 | 29.59 | L | O |
| ATOM | 13088 | O | WAT | L | 76 | 36.137 | 20.620 | −27.755 | 1.00 | 25.94 | L | O |
| ATOM | 13089 | O | WAT | L | 77 | 38.236 | 18.973 | −28.451 | 1.00 | 25.32 | L | O |
| ATOM | 13090 | O | WAT | L | 78 | 37.397 | 16.728 | −30.833 | 1.00 | 30.94 | L | O |
| ATOM | 13091 | O | WAT | L | 79 | −20.096 | 8.381 | 12.510 | 1.00 | 32.67 | L | O |
| ATOM | 13092 | O | WAT | L | 80 | 24.272 | 34.803 | −28.765 | 1.00 | 56.17 | L | O |
| ATOM | 13093 | O | WAT | L | 81 | 12.265 | −1.899 | −9.156 | 1.00 | 40.79 | L | O |
| ATOM | 13094 | O | WAT | L | 82 | 21.784 | 19.019 | 1.479 | 1.00 | 35.04 | L | O |
| ATOM | 13095 | O | WAT | L | 83 | 10.880 | −12.078 | 2.697 | 1.00 | 22.80 | L | O |
| ATOM | 13096 | O | WAT | L | 84 | 68.283 | 36.402 | −46.414 | 1.00 | 34.46 | L | O |
| ATOM | 13097 | O | WAT | L | 85 | 26.902 | 3.013 | −3.460 | 1.00 | 35.87 | L | O |
| ATOM | 13098 | O | WAT | L | 86 | 2.135 | 10.815 | 3.609 | 1.00 | 38.07 | L | O |
| ATOM | 13099 | O | WAT | L | 87 | 40.740 | 39.878 | −40.836 | 1.00 | 45.16 | L | O |
| ATOM | 13100 | O | WAT | L | 88 | 1.416 | 25.914 | 6.608 | 1.00 | 74.99 | L | O |
| ATOM | 13101 | O | WAT | L | 89 | −9.987 | 17.984 | −5.133 | 1.00 | 41.57 | L | O |
| ATOM | 13102 | O | WAT | L | 90 | 10.493 | −3.028 | 5.411 | 1.00 | 35.12 | L | O |
| ATOM | 13103 | O | WAT | L | 91 | 8.715 | −0.933 | 5.130 | 1.00 | 35.66 | L | O |
| ATOM | 13104 | O | WAT | L | 92 | 47.756 | 30.441 | −47.559 | 1.00 | 52.50 | L | O |
| ATOM | 13105 | O | WAT | L | 93 | −3.264 | 8.661 | −14.346 | 1.00 | 31.47 | L | O |
| ATOM | 13106 | O | WAT | L | 94 | 11.672 | 2.930 | 11.218 | 1.00 | 35.21 | L | O |
| ATOM | 13107 | O | WAT | L | 95 | 7.380 | 9.855 | −12.462 | 1.00 | 37.07 | L | O |
| ATOM | 13108 | O | WAT | L | 96 | 0.410 | 35.201 | −7.731 | 1.00 | 57.03 | L | O |
| ATOM | 13109 | O | WAT | L | 97 | 27.144 | 10.297 | −15.801 | 1.00 | 50.79 | L | O |
| ATOM | 13110 | O | WAT | L | 98 | −4.850 | 4.889 | −8.314 | 1.00 | 29.21 | L | O |
| ATOM | 13111 | O | WAT | L | 99 | 17.294 | 27.122 | −16.658 | 1.00 | 37.01 | L | O |
| ATOM | 13112 | O | WAT | L | 100 | 30.478 | 9.314 | −38.344 | 1.00 | 50.11 | L | O |
| ATOM | 13113 | O | WAT | L | 101 | 27.864 | 9.462 | −1.550 | 1.00 | 23.22 | L | O |
| ATOM | 13114 | O | WAT | L | 102 | −3.160 | 24.091 | 2.304 | 1.00 | 53.48 | L | O |
| ATOM | 13115 | O | WAT | L | 103 | −3.466 | −30.565 | 5.183 | 1.00 | 58.26 | L | O |
| ATOM | 13116 | O | WAT | L | 104 | 28.995 | 4.535 | −2.290 | 1.00 | 40.95 | L | O |
| ATOM | 13117 | O | WAT | L | 105 | 40.759 | 42.800 | −56.375 | 1.00 | 40.56 | L | O |
| ATOM | 13118 | O | WAT | L | 106 | 31.883 | 42.070 | −56.497 | 1.00 | 42.35 | L | O |
| ATOM | 13119 | O | WAT | L | 107 | 62.286 | 41.669 | −39.548 | 1.00 | 38.01 | L | O |
| ATOM | 13120 | O | WAT | L | 108 | 33.962 | 25.182 | −4.055 | 1.00 | 45.50 | L | O |
| ATOM | 13121 | O | WAT | L | 109 | −6.361 | 21.548 | −15.093 | 1.00 | 30.63 | L | O |
| ATOM | 13122 | O | WAT | L | 110 | 24.886 | 27.876 | −11.131 | 1.00 | 44.76 | L | O |
| ATOM | 13123 | O | WAT | L | 111 | 11.596 | 23.246 | −26.372 | 1.00 | 49.52 | L | O |
| ATOM | 13124 | O | WAT | L | 112 | −10.198 | −5.357 | −8.343 | 1.00 | 50.27 | L | O |
| ATOM | 13125 | O | WAT | L | 113 | 30.921 | 7.278 | −8.677 | 1.00 | 31.48 | L | O |
| ATOM | 13126 | O | WAT | L | 114 | 17.206 | 30.521 | −20.819 | 1.00 | 43.90 | L | O |
| ATOM | 13127 | O | WAT | L | 115 | 56.588 | 26.779 | −49.889 | 1.00 | 50.50 | L | O |
| ATOM | 13128 | O | WAT | L | 116 | 47.472 | 30.182 | −41.597 | 1.00 | 57.38 | L | O |
| ATOM | 13129 | O | WAT | L | 117 | 5.671 | 26.523 | −19.335 | 1.00 | 49.07 | L | O |
| ATOM | 13130 | O | WAT | L | 118 | 43.255 | 31.271 | −41.927 | 1.00 | 55.33 | L | O |
| ATOM | 13131 | O | WAT | L | 119 | 51.918 | 66.637 | −50.016 | 1.00 | 39.06 | L | O |
| ATOM | 13132 | O | WAT | L | 120 | 37.869 | 13.623 | −19.982 | 1.00 | 66.13 | L | O |
| ATOM | 13133 | O | WAT | L | 121 | −22.709 | 8.256 | −1.043 | 1.00 | 41.97 | L | O |
| ATOM | 13134 | O | WAT | L | 122 | 47.931 | 44.358 | −55.900 | 1.00 | 38.57 | L | O |
| ATOM | 13135 | O | WAT | L | 123 | −8.131 | 19.270 | 2.417 | 1.00 | 59.03 | L | O |
| ATOM | 13136 | O | WAT | L | 124 | 28.499 | 14.665 | 0.309 | 1.00 | 53.05 | L | O |
| ATOM | 13137 | O | WAT | L | 125 | 43.820 | 24.505 | −33.875 | 1.00 | 61.05 | L | O |
| ATOM | 13138 | O | WAT | M | 1 | 2.972 | 10.991 | 37.020 | 1.00 | 17.26 | M | O |
| ATOM | 13139 | O | WAT | M | 2 | 16.080 | 10.253 | 33.924 | 1.00 | 14.83 | M | O |
| ATOM | 13140 | O | WAT | M | 3 | 9.533 | 14.396 | 39.549 | 1.00 | 17.16 | M | O |
| ATOM | 13141 | O | WAT | M | 4 | 8.626 | −3.897 | 57.067 | 1.00 | 29.91 | M | O |
| ATOM | 13142 | O | WAT | M | 5 | −13.161 | 2.078 | 46.854 | 1.00 | 35.87 | M | O |
| ATOM | 13143 | O | WAT | M | 6 | 36.232 | 13.103 | 19.087 | 1.00 | 15.86 | M | O |
| ATOM | 13144 | O | WAT | M | 7 | 35.094 | 15.555 | 19.236 | 1.00 | 10.75 | M | O |
| ATOM | 13145 | O | WAT | M | 8 | 32.692 | 15.880 | 17.632 | 1.00 | 34.85 | M | O |
| ATOM | 13146 | O | WAT | M | 9 | −15.093 | −3.308 | 47.470 | 1.00 | 27.93 | M | O |
| ATOM | 13147 | O | WAT | M | 10 | −11.204 | −3.896 | 50.580 | 1.00 | 44.73 | M | O |
| ATOM | 13148 | O | WAT | M | 11 | 25.588 | 10.840 | 16.602 | 1.00 | 9.37 | M | O |
| ATOM | 13149 | O | WAT | M | 12 | 13.254 | 10.209 | 33.424 | 1.00 | 18.32 | M | O |
| ATOM | 13150 | O | WAT | M | 13 | 12.295 | 9.498 | 43.771 | 1.00 | 16.89 | M | O |
| ATOM | 13151 | O | WAT | M | 14 | 18.720 | 17.398 | 24.027 | 1.00 | 16.06 | M | O |
| ATOM | 13152 | O | WAT | M | 15 | 19.135 | 15.761 | 32.850 | 1.00 | 26.58 | M | O |
| ATOM | 13153 | O | WAT | M | 16 | 5.735 | 16.460 | 36.481 | 1.00 | 22.39 | M | O |
| ATOM | 13154 | O | WAT | M | 17 | 24.571 | 15.617 | 44.330 | 1.00 | 26.71 | M | O |
| ATOM | 13155 | O | WAT | M | 18 | 59.184 | 32.558 | 19.461 | 1.00 | 33.80 | M | O |
| ATOM | 13156 | O | WAT | M | 19 | 27.386 | 41.844 | 42.211 | 1.00 | 39.76 | M | O |
| ATOM | 13157 | O | WAT | M | 20 | 14.743 | 14.433 | 61.983 | 1.00 | 53.31 | M | O |
| ATOM | 13158 | O | WAT | M | 21 | 41.057 | 30.160 | 20.560 | 1.00 | 35.44 | M | O |
| ATOM | 13159 | O | WAT | M | 22 | 26.519 | 18.637 | 20.299 | 1.00 | 25.63 | M | O |
| ATOM | 13160 | O | WAT | M | 23 | 6.672 | −3.536 | 50.269 | 1.00 | 27.33 | M | O |
| ATOM | 13161 | O | WAT | M | 24 | 5.750 | −2.969 | 52.716 | 1.00 | 37.66 | M | O |
| ATOM | 13162 | O | WAT | M | 25 | 17.364 | 7.200 | 53.785 | 1.00 | 19.50 | M | O |
| ATOM | 13163 | O | WAT | M | 26 | 25.084 | −0.288 | 28.049 | 1.00 | 39.40 | M | O |
| ATOM | 13164 | O | WAT | M | 27 | −13.314 | −0.934 | 66.388 | 1.00 | 28.98 | M | O |
| ATOM | 13165 | O | WAT | M | 28 | 29.992 | 25.262 | 25.579 | 1.00 | 19.82 | M | O |
| ATOM | 13166 | O | WAT | M | 29 | 23.896 | −0.150 | 21.642 | 1.00 | 23.70 | M | O |
| ATOM | 13167 | O | WAT | M | 30 | 3.123 | −1.338 | 57.208 | 1.00 | 50.70 | M | O |
| ATOM | 13168 | O | WAT | M | 31 | 8.007 | 16.109 | 31.573 | 1.00 | 38.41 | M | O |
| ATOM | 13169 | O | WAT | M | 32 | −6.795 | 1.036 | 82.007 | 1.00 | 30.61 | M | O |
| ATOM | 13170 | O | WAT | M | 33 | −2.046 | −6.420 | 58.248 | 1.00 | 61.18 | M | O |
| ATOM | 13171 | O | WAT | M | 34 | 32.229 | 24.205 | 33.426 | 1.00 | 24.00 | M | O |
| ATOM | 13172 | O | WAT | M | 35 | 22.664 | 3.903 | 31.838 | 1.00 | 16.72 | M | O |
| ATOM | 13173 | O | WAT | M | 36 | 41.605 | 22.744 | 16.655 | 1.00 | 56.06 | M | O |
| ATOM | 13174 | O | WAT | M | 37 | −39.016 | −1.832 | 72.242 | 1.00 | 32.11 | M | O |
| ATOM | 13175 | O | WAT | M | 38 | −2.534 | −2.520 | 33.426 | 1.00 | 39.33 | M | O |
| ATOM | 13176 | O | WAT | M | 39 | 9.753 | 4.419 | 43.330 | 1.00 | 25.18 | M | O |
| ATOM | 13177 | O | WAT | M | 40 | −11.113 | −16.726 | 83.210 | 1.00 | 26.51 | M | O |
| ATOM | 13178 | O | WAT | M | 41 | 7.165 | 17.390 | 43.062 | 1.00 | 24.66 | M | O |
| ATOM | 13179 | O | WAT | M | 42 | 7.953 | −6.061 | 49.799 | 1.00 | 29.36 | M | O |
| ATOM | 13180 | O | WAT | M | 43 | −29.408 | 5.531 | 76.093 | 1.00 | 30.14 | M | O |
| ATOM | 13181 | O | WAT | M | 44 | 51.396 | 20.867 | 8.899 | 1.00 | 41.40 | M | O |
| ATOM | 13182 | O | WAT | M | 45 | 37.193 | 4.255 | 11.130 | 1.00 | 30.37 | M | O |
| ATOM | 13183 | O | WAT | M | 46 | 14.384 | 10.376 | 57.626 | 1.00 | 19.53 | M | O |
| ATOM | 13184 | O | WAT | M | 47 | 25.570 | 6.733 | 9.651 | 1.00 | 24.56 | M | O |
| ATOM | 13185 | O | WAT | M | 48 | 32.261 | 35.634 | 45.288 | 1.00 | 28.85 | M | O |
| ATOM | 13186 | O | WAT | M | 49 | 13.497 | 20.659 | 52.890 | 1.00 | 34.77 | M | O |
| ATOM | 13187 | O | WAT | M | 50 | 18.533 | 1.399 | 25.838 | 1.00 | 27.45 | M | O |
| ATOM | 13188 | O | WAT | M | 51 | 23.164 | −0.550 | 55.617 | 1.00 | 34.86 | M | O |
| ATOM | 13189 | O | WAT | M | 52 | 48.135 | 20.056 | 26.988 | 1.00 | 40.82 | M | O |
| ATOM | 13190 | O | WAT | M | 53 | 31.670 | 13.740 | 14.812 | 1.00 | 22.22 | M | O |
| ATOM | 13191 | O | WAT | M | 54 | 8.015 | 5.925 | 63.143 | 1.00 | 28.19 | M | O |
| ATOM | 13192 | O | WAT | M | 55 | 42.140 | 7.379 | 23.767 | 1.00 | 22.85 | M | O |
| ATOM | 13193 | O | WAT | M | 56 | −2.068 | 14.844 | 34.357 | 1.00 | 33.98 | M | O |
| ATOM | 13194 | O | WAT | M | 57 | 41.004 | 33.620 | 19.141 | 1.00 | 37.99 | M | O |
| ATOM | 13195 | O | WAT | M | 58 | 25.391 | 16.108 | 19.768 | 1.00 | 35.29 | M | O |
| ATOM | 13196 | O | WAT | M | 59 | 21.043 | 36.611 | 42.841 | 1.00 | 29.60 | M | O |
| ATOM | 13197 | O | WAT | M | 60 | 6.515 | 19.983 | 43.043 | 1.00 | 35.63 | M | O |
| ATOM | 13198 | O | WAT | M | 61 | 9.683 | 17.625 | 39.975 | 1.00 | 20.50 | M | O |
| ATOM | 13199 | O | WAT | M | 62 | 35.806 | 10.961 | 33.196 | 1.00 | 54.03 | M | O |
| ATOM | 13200 | O | WAT | M | 63 | −11.237 | 12.581 | 77.367 | 1.00 | 33.53 | M | O |
| ATOM | 13201 | O | WAT | M | 64 | 62.041 | 30.294 | −59.580 | 1.00 | 30.84 | M | O |
| ATOM | 13202 | O | WAT | M | 65 | 43.341 | 26.666 | 24.732 | 1.00 | 33.72 | M | O |
| ATOM | 13203 | O | WAT | M | 66 | 44.431 | 23.305 | 23.919 | 1.00 | 31.90 | M | O |
| ATOM | 13204 | O | WAT | M | 67 | −5.495 | 8.782 | 70.038 | 1.00 | 28.75 | M | O |
| ATOM | 13205 | O | WAT | M | 68 | 21.694 | 7.375 | 12.691 | 1.00 | 24.55 | M | O |
| ATOM | 13206 | O | WAT | M | 69 | −0.980 | −4.946 | 52.317 | 1.00 | 43.79 | M | O |
| ATOM | 13207 | O | WAT | M | 70 | −36.930 | −5.197 | 76.180 | 1.00 | 25.40 | M | O |
| ATOM | 13208 | O | WAT | M | 71 | 23.272 | 28.235 | 46.271 | 1.00 | 37.16 | M | O |
| ATOM | 13209 | O | WAT | M | 72 | 22.850 | 30.460 | 44.479 | 1.00 | 49.19 | M | O |
| ATOM | 13210 | O | WAT | M | 73 | 18.371 | 9.760 | 50.482 | 1.00 | 31.90 | M | O |
| ATOM | 13211 | O | WAT | M | 74 | −35.999 | −1.144 | 84.884 | 1.00 | 34.62 | M | O |
| ATOM | 13212 | O | WAT | M | 75 | −23.809 | 1.490 | 74.262 | 1.00 | 43.15 | M | O |
| ATOM | 13213 | O | WAT | M | 76 | −38.306 | −0.741 | 67.586 | 1.00 | 28.64 | M | O |
| ATOM | 13214 | O | WAT | M | 77 | 4.633 | 18.248 | 46.426 | 1.00 | 38.60 | M | O |
| ATOM | 13215 | O | WAT | M | 78 | −17.941 | −5.240 | 65.926 | 1.00 | 27.52 | M | O |
| ATOM | 13216 | O | WAT | M | 79 | −3.502 | 2.369 | 59.028 | 1.00 | 39.63 | M | O |
| ATOM | 13217 | O | WAT | M | 80 | −21.426 | −3.876 | 57.788 | 1.00 | 32.89 | M | O |
| ATOM | 13218 | O | WAT | M | 81 | 34.524 | 16.577 | 21.671 | 1.00 | 22.09 | M | O |
| ATOM | 13219 | O | WAT | M | 82 | 41.105 | 37.818 | 31.805 | 1.00 | 25.92 | M | O |
| ATOM | 13220 | O | WAT | M | 83 | −30.309 | −10.500 | 81.593 | 1.00 | 33.83 | M | O |
| ATOM | 13221 | O | WAT | M | 84 | −4.772 | 13.130 | 45.159 | 1.00 | 26.43 | M | O |
| ATOM | 13222 | O | WAT | M | 85 | −31.485 | 4.482 | 67.497 | 1.00 | 39.31 | M | O |
| ATOM | 13223 | O | WAT | M | 86 | 37.585 | 13.376 | 29.166 | 1.00 | 33.55 | M | O |
| ATOM | 13224 | O | WAT | M | 87 | 35.759 | 15.294 | 29.001 | 1.00 | 24.17 | M | O |
| ATOM | 13225 | O | WAT | M | 88 | 37.540 | 18.709 | 29.629 | 1.00 | 35.72 | M | O |
| ATOM | 13226 | O | WAT | M | 89 | 43.442 | 16.668 | 22.965 | 1.00 | 44.85 | M | O |
| ATOM | 13227 | O | WAT | M | 90 | −2.867 | −8.361 | 46.114 | 1.00 | 36.13 | M | O |
| ATOM | 13228 | O | WAT | M | 91 | −3.514 | 9.300 | 65.719 | 1.00 | 40.43 | M | O |
| ATOM | 13229 | O | WAT | M | 92 | −13.464 | −14.635 | 66.959 | 1.00 | 29.33 | M | O |
| ATOM | 13230 | O | WAT | M | 93 | −9.997 | 7.032 | 37.001 | 1.00 | 32.34 | M | O |
| ATOM | 13231 | O | WAT | M | 94 | −2.604 | 13.561 | 55.123 | 1.00 | 34.31 | M | O |
| ATOM | 13232 | O | WAT | M | 95 | −4.993 | 8.761 | 50.600 | 1.00 | 48.55 | M | O |
| ATOM | 13233 | O | WAT | M | 96 | −21.407 | −27.767 | 69.101 | 1.00 | 72.63 | M | O |
| ATOM | 13234 | O | WAT | M | 97 | 54.975 | 21.857 | 27.783 | 1.00 | 37.55 | M | O |
| ATOM | 13235 | O | WAT | M | 98 | 25.564 | 39.708 | 31.861 | 1.00 | 33.85 | M | O |
| ATOM | 13236 | O | WAT | M | 99 | 35.136 | −1.518 | 13.228 | 1.00 | 23.38 | M | O |
| ATOM | 13237 | O | WAT | M | 100 | 34.093 | −4.020 | 12.884 | 1.00 | 37.20 | M | O |
| ATOM | 13238 | O | WAT | M | 101 | −5.377 | −6.413 | 77.642 | 1.00 | 38.55 | M | O |
| ATOM | 13239 | O | WAT | M | 102 | 12.861 | 32.403 | 21.228 | 1.00 | 52.21 | M | O |
| ATOM | 13240 | O | WAT | M | 103 | 7.225 | 11.162 | 51.295 | 1.00 | 28.26 | M | O |
| ATOM | 13241 | O | WAT | M | 104 | 35.071 | 17.849 | 29.092 | 1.00 | 44.60 | M | O |
| ATOM | 13242 | O | WAT | M | 105 | 11.817 | 19.179 | 63.059 | 1.00 | 34.70 | M | O |
| ATOM | 13243 | O | WAT | M | 106 | 32.648 | 18.621 | 22.820 | 1.00 | 42.23 | M | O |
| ATOM | 13244 | O | WAT | M | 107 | −9.379 | 14.927 | 44.289 | 1.00 | 58.55 | M | O |
| ATOM | 13245 | O | WAT | M | 108 | 17.200 | 17.647 | 58.185 | 1.00 | 36.41 | M | O |
| ATOM | 13246 | O | WAT | M | 109 | 59.157 | 36.676 | 19.444 | 1.00 | 30.29 | M | O |
| ATOM | 13247 | O | WAT | M | 110 | −11.585 | 0.430 | 62.879 | 1.00 | 56.29 | M | O |
| ATOM | 13248 | O | WAT | M | 111 | 60.424 | 32.052 | 1.950 | 1.00 | 49.74 | M | O |
| ATOM | 13249 | O | WAT | M | 112 | 16.323 | 35.408 | 38.308 | 1.00 | 39.77 | M | O |
| ATOM | 13250 | O | WAT | M | 113 | −26.722 | −4.456 | 52.309 | 1.00 | 36.33 | M | O |
| ATOM | 13251 | O | WAT | M | 114 | −7.318 | −5.784 | 53.190 | 1.00 | 37.65 | M | O |
| ATOM | 13252 | O | WAT | M | 115 | 27.895 | 44.131 | 37.115 | 1.00 | 34.80 | M | O |
| ATOM | 13253 | O | WAT | M | 116 | −27.323 | 5.239 | 82.024 | 1.00 | 38.61 | M | O |
| ATOM | 13254 | O | WAT | M | 117 | 67.084 | 14.318 | 19.316 | 1.00 | 45.53 | M | O |
| ATOM | 13255 | O | WAT | M | 118 | 0.445 | 17.961 | 59.565 | 1.00 | 35.57 | M | O |
| ATOM | 13256 | O | WAT | M | 119 | 11.989 | 8.876 | 49.649 | 1.00 | 39.17 | M | O |
| ATOM | 13257 | O | WAT | M | 120 | −5.221 | −6.028 | 71.011 | 1.00 | 53.48 | M | O |
| ATOM | 13258 | O | WAT | M | 121 | 4.721 | 19.076 | 36.706 | 1.00 | 35.99 | M | O |
| ATOM | 13259 | O | WAT | M | 122 | 61.552 | 26.146 | 10.925 | 1.00 | 39.44 | M | O |
| ATOM | 13260 | O | WAT | M | 123 | 17.960 | 16.256 | 46.615 | 1.00 | 29.09 | M | O |
| ATOM | 13261 | O | WAT | M | 124 | 70.186 | 36.591 | 22.658 | 1.00 | 49.87 | M | O |
| ATOM | 13262 | O | WAT | M | 125 | 39.229 | 36.826 | 30.241 | 1.00 | 45.68 | M | O |
| ATOM | 13263 | O | WAT | M | 126 | −19.445 | −6.966 | 81.320 | 1.00 | 32.01 | M | O |
| ATOM | 13264 | O | WAT | M | 127 | 15.916 | 32.063 | 44.851 | 1.00 | 43.62 | M | O |
| ATOM | 13265 | O | WAT | M | 128 | 11.322 | −7.313 | 44.563 | 1.00 | 54.62 | M | O |
| ATOM | 13266 | O | WAT | M | 129 | −7.149 | 5.989 | 57.893 | 1.00 | 43.82 | M | O |
| ATOM | 13267 | O | WAT | M | 130 | −5.136 | 13.045 | 81.339 | 1.00 | 40.11 | M | O |
| ATOM | 13268 | O | WAT | M | 131 | 44.978 | 5.329 | 9.629 | 1.00 | 49.46 | M | O |
| ATOM | 13269 | O | WAT | M | 132 | 44.752 | 9.615 | 20.892 | 1.00 | 29.52 | M | O |
| ATOM | 13270 | O | WAT | M | 133 | 8.742 | 19.638 | 32.610 | 1.00 | 39.87 | M | O |
| ATOM | 13271 | O | WAT | M | 134 | 16.232 | 26.367 | 20.946 | 1.00 | 34.85 | M | O |
| ATOM | 13272 | O | WAT | M | 135 | 22.922 | 11.104 | 17.235 | 1.00 | 33.63 | M | O |
| ATOM | 13273 | O | WAT | M | 136 | 24.960 | −1.457 | 59.007 | 1.00 | 68.41 | M | O |
| ATOM | 13274 | O | WAT | M | 137 | 1.869 | 1.672 | 29.865 | 1.00 | 41.94 | M | O |
| ATOM | 13275 | O | WAT | M | 138 | −15.464 | −1.104 | 82.666 | 1.00 | 32.75 | M | O |
| ATOM | 13276 | O | WAT | M | 139 | −13.376 | 17.612 | 66.240 | 1.00 | 54.95 | M | O |
| ATOM | 13277 | O | WAT | M | 140 | −18.639 | 9.462 | −13.658 | 1.00 | 51.81 | M | O |
| ATOM | 13278 | O | WAT | M | 141 | 39.632 | 0.025 | 16.731 | 1.00 | 46.08 | M | O |
| ATOM | 13279 | O | WAT | M | 142 | 58.056 | 35.423 | 3.748 | 1.00 | 53.53 | M | O |
| ATOM | 13280 | O | WAT | M | 143 | 66.697 | 10.360 | 22.431 | 1.00 | 46.06 | M | O |
| ATOM | 13281 | O | WAT | M | 144 | −5.312 | −0.209 | 69.588 | 1.00 | 49.56 | M | O |
| ATOM | 13282 | O | WAT | M | 145 | 51.287 | 6.533 | 23.713 | 1.00 | 40.97 | M | O |
| ATOM | 13283 | O | WAT | M | 146 | 11.367 | 14.962 | 24.643 | 1.00 | 52.35 | M | O |
| ATOM | 13284 | O | WAT | M | 147 | 21.273 | −0.950 | 20.459 | 1.00 | 45.28 | M | O |
| ATOM | 13285 | O | WAT | M | 148 | 18.659 | 29.039 | 20.114 | 1.00 | 51.02 | M | O |
| ATOM | 13286 | O | WAT | M | 149 | −12.672 | 12.451 | 80.714 | 1.00 | 56.60 | M | O |
| ATOM | 13285 | N | ARG | L | 24 | 19.216 | 1.271 | −12.787 | 0.50 | 27.08 | L | N |
| ATOM | 13286 | CA | ARG | L | 24 | 19.737 | 1.305 | −14.147 | 0.50 | 27.75 | L | C |
| ATOM | 13287 | CB | ARG | L | 24 | 19.852 | −0.120 | −14.710 | 0.50 | 29.27 | L | C |
| ATOM | 13288 | CG | ARG | L | 24 | 21.015 | −0.336 | −15.688 | 0.50 | 29.72 | L | C |
| ATOM | 13289 | CD | ARG | L | 24 | 20.569 | −0.427 | −17.146 | 0.50 | 32.60 | L | C |
| ATOM | 13290 | NE | ARG | L | 24 | 21.710 | −0.397 | −18.062 | 0.50 | 30.59 | L | N |
| ATOM | 13291 | CZ | ARG | L | 24 | 21.618 | −0.164 | −19.366 | 0.50 | 31.75 | L | C |
| ATOM | 13292 | NH1 | ARG | L | 24 | 20.432 | 0.047 | −19.923 | 0.50 | 34.53 | L | N |
| ATOM | 13293 | NH2 | ARG | L | 24 | 22.716 | −0.073 | −20.102 | 0.50 | 32.33 | L | N |
| ATOM | 13294 | C | ARG | L | 24 | 18.806 | 2.140 | −15.024 | 0.50 | 29.20 | L | C |
| ATOM | 13295 | O | ARG | L | 24 | 17.601 | 1.887 | −15.072 | 0.50 | 34.86 | L | O |
| ATOM | 13296 | N | CYS | X | 140 | 50.957 | 50.232 | −44.137 | 0.50 | 23.03 | X | N |
| ATOM | 13297 | CA | CYS | X | 140 | 52.075 | 50.202 | −43.202 | 0.50 | 22.69 | X | C |
| ATOM | 13298 | C | CYS | X | 140 | 52.086 | 48.887 | −42.450 | 0.50 | 20.83 | X | C |
| ATOM | 13299 | O | CYS | X | 140 | 51.073 | 48.486 | −41.881 | 0.50 | 23.74 | X | O |
| ATOM | 13300 | CB | CYS | X | 140 | 51.971 | 51.350 | −42.179 | 0.50 | 19.29 | X | C |
| ATOM | 13301 | SG | CYS | X | 140 | 52.828 | 52.874 | −42.688 | 0.50 | 25.25 | X | S |
| ATOM | 13302 | N | CYS | X | 195 | 51.658 | 57.246 | −42.296 | 0.50 | 23.40 | X | N |
| ATOM | 13303 | CA | CYS | X | 195 | 50.955 | 56.501 | −41.264 | 0.50 | 23.47 | X | C |
| ATOM | 13304 | C | CYS | X | 195 | 51.543 | 56.888 | −39.916 | 0.50 | 24.63 | X | C |
| ATOM | 13305 | O | CYS | X | 195 | 52.736 | 57.163 | −39.799 | 0.50 | 25.36 | X | O |
| ATOM | 13306 | CB | CYS | X | 195 | 51.017 | 54.980 | −41.480 | 0.50 | 19.68 | X | C |
| ATOM | 13307 | SG | CYS | X | 195 | 52.601 | 54.157 | −41.131 | 0.50 | 18.28 | X | S |
| ATOM | 13308 | N | CYS | Z | 140 | −26.817 | −6.049 | 70.413 | 0.50 | 25.48 | Z | N |
| ATOM | 13309 | CA | CYS | Z | 140 | −27.243 | −7.010 | 69.406 | 0.50 | 22.69 | Z | C |
| ATOM | 13310 | C | CYS | Z | 140 | −26.076 | −7.509 | 68.567 | 0.50 | 23.17 | Z | C |
| ATOM | 13311 | O | CYS | Z | 140 | −25.236 | −6.728 | 68.111 | 0.50 | 23.46 | Z | O |
| ATOM | 13312 | CB | CYS | Z | 140 | −28.305 | −6.396 | 68.480 | 0.50 | 23.82 | Z | C |
| ATOM | 13313 | SG | CYS | Z | 140 | −30.013 | −6.600 | 69.085 | 0.50 | 27.71 | Z | S |
| ATOM | 13314 | N | CYS | Z | 195 | −33.602 | −3.695 | 69.257 | 0.50 | 21.30 | Z | N |
| ATOM | 13315 | CA | CYS | Z | 195 | −32.660 | −3.235 | 68.256 | 0.50 | 21.93 | Z | C |
| ATOM | 13316 | C | CYS | Z | 195 | −33.287 | −3.570 | 66.898 | 0.50 | 23.73 | Z | C |
| ATOM | 13317 | O | CYS | Z | 195 | −33.835 | −4.658 | 66.701 | 0.50 | 22.98 | Z | O |
| ATOM | 13318 | CB | CYS | Z | 195 | −31.292 | −3.913 | 68.414 | 0.50 | 22.55 | Z | C |
| ATOM | 13319 | SG | CYS | Z | 195 | −31.154 | −5.597 | 67.743 | 0.50 | 26.19 | Z | S |
| END | | | | | | | | | | | | |

### Example 13

### Detection of Anti-D3H44 Antibodies in Chimpanzees

Chimpanzees were treated with D3H44-F(ab′)₂ 1 mg/kg day 0, 0.75 mg/kg on day 1 and 2, 0.5 mg/kg day 3 and 4, 0.25 mg/kg day 5, i.v. Plasma (citrated) samples were prepared from blood drawn prior to treatment with D3H44-F(ab′)₂, and blood drawn on days 21 and 43.

Competitive ELISA assays were performed by coating microtiter plates with D3H44 antibody, incubating the coated plates with a mixture of chimpanzee plasma and 6A6 monoclonal antibody or D3 monoclonal antibody. The procedure was generally carried out as follows: Nunc Maxisorp plates were coated with D3H44F(ab′)2 (2 ug/ml in 50 mM carbonate buffer, pH 9.6, 100 μl/well) at 4° C. overnight or at room temperature for 2 hours. The wells were blocked by adding 150 μl 0.05% BSA in PBS for 1 hour. Next, the wells were washed with PBS −0.05% Tween20 5 times. Afterwards, a mixture of 6A6 antibody in ELISA buffer (PBS −0.05% BSA and 0.05% Tween20)(50 μl/ml, 10 μl/ml, 2 μl/ml, 0.4 μl/ml final concentration) or D3 antibody)(50 μl/ml, 10 μl/ml, 2 μl/ml, 0.4 μl/ml final concentration) and a 1:300 dilution of chimpanzee plasma in ELISA buffer were added to the wells for 2 hours at room temperature. Then, the wells were washed with PBS −0.05% Tween20 10 times. Bound chimpanzee anti-D3H44 antibodies were detected using an anti-human Fc antibody-horse radish peroxidase (HRP) conjugate followed by washing, adding 3,3′,5,5′-tetramethyl benzidine (Kirkegaard & Perry Laboratories, Gaithersburg, Md.) as the substrate and monitoring the cleaved substrate at OD 450 nm. The D3 antibody served as a control.

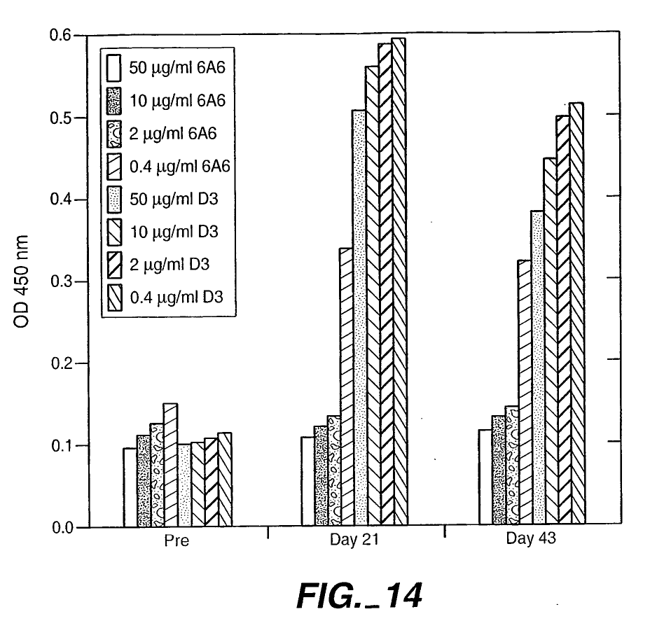

FIG. 14 shows that anti-D3H44 antibodies were observed in chimps treated with D3H44. These antibodies showed little or no binding to D3H44 in the presence of 6A6 antibody as compared to D3 antibody.

1. An AATF antibody that specifically binds to a variable region of an anti-tissue factor antibody (ATF antibody) that specifically binds to an extracellular domain of a tissue factor.

2. The AATF antibody according to claim 1, wherein the ATF antibody binds to a region of tissue factor that interacts with Factor X.

3. The AATF antibody according to claim 2, wherein the region is within residues 156-204 of a human tissue factor.

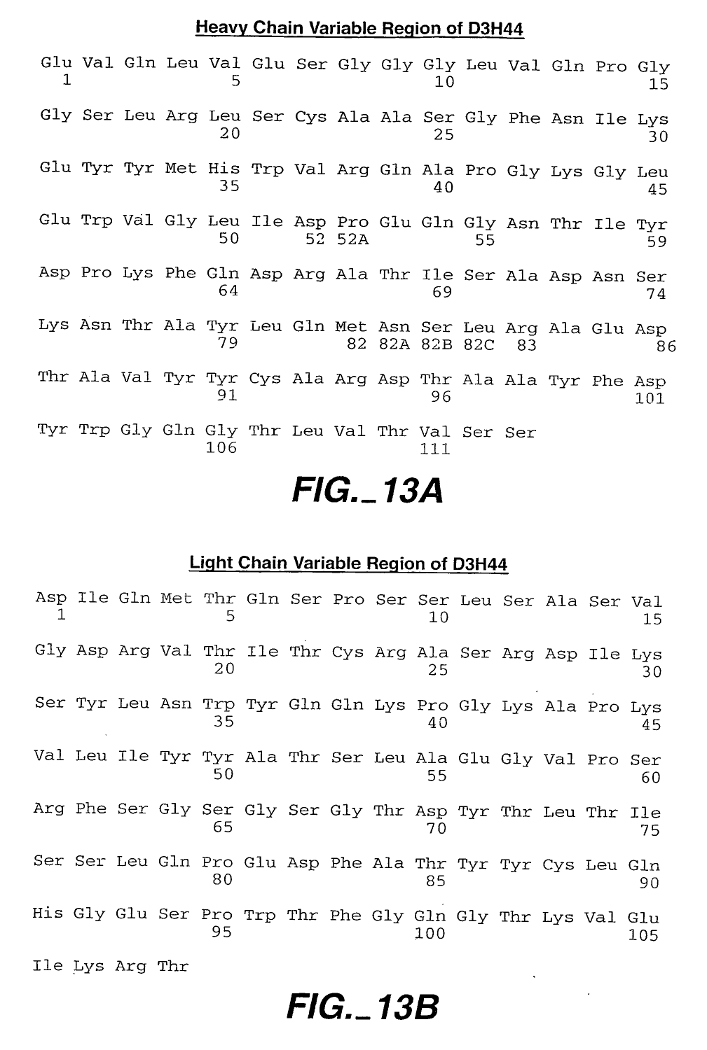

4. The AATF antibody according to claim 1, wherein the ATF antibody comprises an amino acid sequence selected from the group consisting of the sequence of FIG. 13A and the sequence of FIG. 13B.

5. The AATF antibody according to claim 4, wherein the AATF antibody binds to a region having a sequence selected from the group consisting of:
(a) residues 26-35 of FIG. 13A;
(b) residues 50-65 of FIG. 13A;
(c) residues 95-102 of FIG. 13A;
(d) residues 24-34 of FIG. 13B;
(e) residues 50-56 of FIG. 13B; and
(f) residues 89-97 of FIG. 13B;

6. The AATF antibody according to claim 5, wherein the ATF antibody binds to the regions recited in (a)-(f).

7. The AATF antibody according claim 1, wherein the AATF antibody binds to the ATF antibody with an equilibrium dissociation constant (K_{D}) selected from the group consisting of 1 μM or less, 500 nM or less, 100 nM or less, 50 nM or less, 10 nM or less, 5 nM or less and 1 nM or less.

8. The AATF antibody according to claim 1, wherein the antibody inhibits binding of an ATF antibody to a tissue factor.

9. The AATF antibody according to claim 4 that comprises amino acid sequences:
(a) residues 31-35 of FIG. 11B;
(b) residues 50-65 of FIG. 11B;
(c) residues 94-102 of FIG. 11B; or
a variant of the AATF antibody that binds the ATF antibody.

10. The AATF antibody according claim 9, wherein the AATF antibody further comprises amino acid sequences:
(a) residues 26-35 of FIG. 12B;
(b) residues 50-56 of FIG. 12B; and
(c) residues 89-97 of FIG. 12B; or
a variant of the AATF antibody that binds the ATF antibody.

11. The AATF antibody according to claim 4, where the AATF antibody comprises the sequence of FIG. 11B, or a variant thereof.

12. The AATF antibody according to claim 4, wherein the antibody comprises comprises the sequence of FIG. 12B, or a variant thereof.

13. The AATF antibody according to claim 4, where the AATF antibody comprises (a) a variable heavy chain region comprising the sequence of FIG. 11B, or a variant thereof, and (b) a variable light chain region comprising the sequence of FIG. 12B, or a variant thereof.

14. The AATF antibody according to claim 1, wherein the AATF antibody is a monoclonal antibody.

15. The AATF antibody according to claim 1, wherein the AATF antibody is selected from the group consisting of a chimeric antibody, a humanized antibody and an antibody fragment.

16. The AATF antibody according to claim 15, wherein the AATF antibody has a framework region that has been derived from one or more human antibodies.

17. A monoclonal antibody that binds essentially the same epitope as an antibody produced by the hybridoma cell line having the ATCC Deposit No. PTA-5066, deposited Mar. 20, 2003 as designation 6A6.3E11.2E12 in the American Type Culture Collection (ATCC), 10801 University Blvd., Manassas, Va. 20110-2209, USA.

18. A monoclonal antibody having the biological characteristics of a 6A6 antibody produced by the hybridoma cell line having the ATCC Deposit No. PTA-5066, deposited Mar. 20, 2003 as designation 6A6.3E11.2E12 in the American Type Culture Collection (ATCC), 10801 University Blvd., Manassas, Va. 20110-2209, USA.

19. The monoclonal antibody according to claim 18 that is the antibody produced by the hybridoma cell line having the ATCC Deposit No. PTA-5066.

20. The AATF antibody according to claim 1, wherein the AATF antibody is further conjugated to an agent selected from the group consisting of a growth inhibitory agent, a cytotoxic agent, a detection agent, an agent that improves the bioavailability of the antibody and an agent that improves the half-life of the antibody.

21. The AATF antibody according to claim 20, wherein said cytotoxic agent is selected from the group consisting of a toxin, an antibiotic and a radioactive isotope.

22. The AATF antibody according to claim 1, wherein said AATF antibody is produced in bacteria or in a mammalian cell.

23. The AATF antibody according to claim 22, wherein said mammalian cell is a CHO cell.

24. An AATF polypeptide that binds to an ATF antibody comprising the sequences of FIG. 13A and FIG. 13B, wherein the polypeptide comprises sequences:
(a) residues 31-35 of FIG. 11B;
(b) residues 50-65 of FIG. 11B;
(c) residues 94-102 of FIG. 11B;
(d) residues 26-35 of FIG. 12B;
(e) residues 50-56 of FIG. 12B; and
(f) residues 89-97 of FIG. 12B; or
a variant of the AATF polypeptide that binds to an antibody comprising the sequences of FIG. 13A and FIG. 13B.

25. The AATF polypeptide according to claim 24, wherein the AATF polypeptide is selected from the group consisting of a chimeric antibody, a humanized antibody and an antibody fragment.

26. A AATF polypeptide comprising binding site described by the structural coordinates of amino acid residues T29, S30, Y32, W91, S93, W96 of a light chain according to Table 7 and amino acid residues Y27, H32, L50, H52, H53, N54, R94, E95, F97, R98, Y99, Y100A of a heavy chain according to Table 7.

27. A AATF polypeptide comprising a sequence starting at one of amino acid residues G31 to H32 and ending at one of amino acid residues Y100A to Y102 as set forth in FIG. 11B.

28. A crystalline form of an antibody having the atomic coordinates recited in Table 8.

29. A crystalline form of a protein complex having the atomic coordinates recited in Table 8.

30. A crystalline form of a protein complex having unit cell parameters of a=78.9 Å, b=85.8 Å, c=92.4 Å, α=77.5°, β=75.9° and γ=63.3°.

31. A nucleic acid molecule encoding the antibody according claim 1.

32. A nucleic acid molecule encoding the polypeptide according to claim 24.

33. A vector comprising the nucleic acid molecule according to claim 31.

34. A host cell comprising the vector according to claim 33.

35. A composition comprising the antibody according to claim 1, optionally, further comprising a second therapeutic agent.

36. A composition comprising the polypeptide according to claim 24 further comprising a second therapeutic agent.

37. The composition according to claim 35, wherein the second therapeutic agent is a procoagulant.

38. The composition according to claim 37, wherein the procoagulant is NovoSeven®.

39. A composition comprising the nucleic acid molecule according to claim 31.

40. The composition according to claim 35, further comprising a pharmaceutically acceptable carrier.

41. An article of manufacture comprising:
(a) a composition of matter comprising an antibody according to claim 1, a polypeptide according to claim 24, an inactive Tissue Factor (iTF);
(b) a container containing said composition;
(c) a label affixed to said container, or a package insert included in said container referring to the use of said antibody, said polypeptide, or said iTF in the treatment of a hypercoagulable state, in the treatment of an inflammatory disorder or as a diagnostic agent;
and optionally, further comprising an anti-tissue factor antibody.

42. A method for producing an antibody comprising the step of culturing a host cell comprising a vector comprising the nucleic acid molecule according to claim 31.

43. The method according to claim 42, wherein the cell is a bacterial cell or a mammalian cell.

44. The method according to claim 47, wherein the mammalian cell is a CHO cell.

45. A method for treating a hypercoagulable state in a mammal in need of treatment therefor comprising the step of administering a therapeutically effective amount of an anti-tissue factor antibody and subsequently a therapeutically effective amount of an antagonist agent selected from the group consisting of the antibody according to claim 1, the polypeptide according to claim 25 or an inactive tissue factor (iTF).

46. A method for treating an inflammation in a mammal in need of treatment therefor comprising the step of administering a therapeutically effective amount of an anti-tissue factor antibody and subsequently a therapeutically effective amount an antagonist agent selected from the group consisting of the antibody according to claim 1, the polypeptide according to claim 24 or an inactive tissue factor (iTF).

47. The method according to claim 45, wherein the mammal is suffering from an illness selected from the group consisting of stroke, atherosclerosis, restenosis, sepsis, septicemia, acute coronary syndrome, disseminated intravascular coagulation (DIC), myocardial infarction, deep vein thrombosis, sickle cell disease, arterial thrombosis, venous thromboembolism, hypertension, adult respiratory distress syndrome (ARDS) and cancer.


SEQUENCE LISTING (15 sequences)

SEQ ID NO: 1 - DNA, 1557 bp, Artificial
  6A6Ch-Fab Nucleotide Sequence
atgaagaagaatatcgcatttcttcttgcatctatgttcgttttttctattgctacaaatgcatacgctcaggctgttgtgactcaggaatctgcactcacctcatcacctggtgaaacagtcacactcacttgtcgctcaagtactggggctgttacaactagtaactatgccaactgggtccaagaaaaaccagatcatttattcactggtgtaataggtggcaccaacaaccgagctccaggtgttcctgccagattctcaggctccctgattggagacaaggctgccctcaccatcacaggggcacagactgaggatgaggcaatatatttctgtgctctctggtacagcaaccattgggtgttcggtggaggaaccaaactcactgtcctacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaattaaatcctctacgccggacgcatcgtggcgctagtacgcaagttcacgtaaaaacggtatctagaggttgaggtgattttatgaaaaagaatatcgcatttcctcttgcatctatgttcgttttttctattgctacaaacgcgtacgctgaggttcagctgcaacagtctggacctgagctggtgaagcctggagcttcagtgaagatatcctgtaaggcttctggttactcattcactggccacctcttgaactgggtgaagcagagccatggaaagaaccttgagtggattggacttgttcatcctcacaatggtgctattacctacaaccagaagttcaaggacaaggccacattaactgtagacaggtcatccaccacagcctacattgagctcgtccgtctgacatctaatgactctgcagtctattactgtgcaagagaggactttaggtaccactattctatggactactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatga

SEQ ID NO: 2 - DNA, 327 bp, Artificial
  Light Chain Variable Region of 6A6
caggctgttgtgactcaggaatctgcactcacctcatcacctggtgaaacagtcacactcacttgtcgctcaagtactggggctgttacaactagtaactatgccaactgggtccaagaaaaaccagatcatttattcactggtgtaataggtggcaccaacaaccgagctccaggtgttcctgccagattctcaggctccctgattggagacaaggctgccctcaccatcacaggggcacagactgaggatgaggcaatatatttctgtgctctctggtacagcaaccattgggtgttcggtggaggaaccaaactcactgtccta

SEQ ID NO: 3 - protein, 109 aa, Artificial
  Light Chain Variable Region of 6A6
QAVVTQESALTSSPGETVTLTCRSSTGAVTTSNYANWVQEKPDHLFTGVIGGTNNRAPGVPARFSGSLIGDKAALTITGAQTEDEAIYFCALWYSNHWVFGGGTKLTVL

SEQ ID NO: 4 - DNA, 360 bp, Artificial
  Heavy Chain Variable Region of 6A6
gaggttcagctgcaacagtctggacctgagctggtgaagcctggagcttcagtgaagatatcctgtaaggcttctggttactcattcactggccacctcttgaactgggtgaagcagagccatggaaagaaccttgagtggattggacttgttcatcctcacaatggtgctattacctacaaccagaagttcaaggacaaggccacattaactgtagacaggtcatccaccacagcctacattgagctcgtccgtctgacatctaatgactctgcagtctattactgtgcaagagaggactttaggtaccactattctatggactactggggtcaaggaacctcagtcaccgtctcctca

SEQ ID NO: 5 - protein, 120 aa, Artificial
  Heavy Chain Variable Region of 6A6
EVQLQQSGPELVKPGASVKISCKASGYSFTGHLLNWVKQSHGKNLEWIGLVHPHNGAITYNQKFKDKATLTVDRSSTTAYIELVRLTSNDSAVYYCAREDFRYHYSMDYWGQGTSVTVSS

SEQ ID NO: 6 - protein, 117 aa, Artificial
  Heavy Chain Variable Region of D3H44
EVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSS

SEQ ID NO: 7 - protein, 109 aa, Artificial
  Light Chain Variable Region of D3H44
DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRT

SEQ ID NO: 8 - protein, 25 aa, Artificial
  N-Terminal Heavy Chain Sequence of 6A6 Monoclonal Antibody
EVQLQQSGPELVKPGASVKISCKAS

SEQ ID NO: 9 - protein, 15 aa, Artificial
  N-Terminal Light Chain Sequence of 6A6 Monoclonal Antibody
QAVVTQESALTSMPG

SEQ ID NO: 10 - DNA, 30 bp, Artificial
  6A6 Heavy Chain Forward Primer
tcacgcgtacgctgaggtycagctgcarca

SEQ ID NO: 11 - DNA, 40 bp, Artificial
  6A6 Heavy Chain Reverse Primer
atgggcccgtcgttttggctgaggagacdgtgasmrdrgt

SEQ ID NO: 12 - DNA, 33 bp, Artificial
  6A6 Heavy Chain Reverse Primer
atgggcccgtcgttttggctgcagagacdgtga

SEQ ID NO: 13 - DNA, 33 bp, Artificial
  6A6 Heavy Chain Reverse Primer
atgggcccgtcgttttggctgaggagacdgtga

SEQ ID NO: 14 - DNA, 32 bp, Artificial
  6A6 Light Chain Forward Primer
caaatgcatacgctcaggctgttgtgactcag

SEQ ID NO: 15 - DNA, 32 bp, Artificial
  6A6 Light Chain Reverse Primer
gccacggtccgtaggacagtsastttggttcc